SEQUENCE LISTING (388 sequences)

SEQ ID NO: 1 - DNA, 305 bp, Homo sapiens
  source 1..305: mol_type = other DNA
cagcaggcgcgctcccggcgaatctgcctgaatcgccgtgaatgcggtggggtgcagggcaggggctggttttctcagccggtcttggcttttctctttctctcctgctccaccagcagcccctccgcgggtcccatgggctccgcgctcagaacagcccggaaccaggcgccgctcgccgctcgctgggggccacccgcctctccccggaacagcctcccgcgggcctcttggcctcgcactggcgccctcacccacacatcgtccctttatccgctcagacgctgcaaagggccttctgtctc

SEQ ID NO: 2 - DNA, 336 bp, Homo sapiens
  source 1..336: mol_type = other DNA
gctttggatttatcctcattggctaaatccctcctgaaacatgaaactgaaacaaagccctgaaccccctcaggctgaaaagacaaaccccgcctgaggccgggtcccgctccccacctggagggacccaattctgggcgccttctggcgacggtccctgctagggacgctgcgctctccgagtgcgagttttcgccaaactgataaagcacgcagaaccgcaatccccaaactaacactgaacccggacccgcgatccccaaactgacaagggacccggaacagcgacccccaaaccgacacgggactcgggaaccgctatctccaaagggcagc

SEQ ID NO: 3 - DNA, 139 bp, Homo sapiens
  source 1..139: mol_type = other DNA
tttccacaacagggagccagcattgaggcgcccagatggcatctgctggaaatcacgggccgctggtgaagcaccacgccttacccgacgtggggaggtgatcccccacctcatcccacccccttctgtctgtctcctt

SEQ ID NO: 4 - DNA, 292 bp, Homo sapiens
  source 1..292: mol_type = other DNA
gctggacaaggagcgctcactgtagctctgctgtggattgtgttggggcgaagagatgggtaagaggtcaaagtcgtaggattctggcgaccgcctaccaagggattgggtccacagcacagaggtctgatcgcttccttctctgctctgccacctccagacagcagctctaaccagctgcccagcagcaagaggatgcgcacggctttcaccagcacgcagctgctagagctggagcgcgagttcgcttctaatatgtacctgtcccgcctacgtcgcatcgagatcgcga

SEQ ID NO: 5 - DNA, 190 bp, Homo sapiens
  source 1..190: mol_type = other DNA
tgcctgacactgaccccaggcgcagccaggaggggctttgtgcgggagagggagggggaccccagcttgcctggggtccacgggactctcttcttcctagttcactttcttgctaaggcgaaggtcctgaggcaggacgagggctgaactgcgctgcaatcgtccccacctccagcgaaacccagttgac

SEQ ID NO: 6 - DNA, 706 bp, Homo sapiens
  source 1..706: mol_type = other DNA
tcggcggagagacctcgaggagagtatggggaaaggaatgaatgctgcggagcgcccctctgggctccacccaagcctcggaggcgggacggtgggctccgtcccgaccccttaggcagctggaccgatacctcctggatcagaccccacaggaagactcgcgtggggcccgatatgtgtacttcaaactctgagcggccaccctcagccaactggccagtggatgcgaatcgtgggccctgaggggcgagggcgctcggaactgcatgcctgtgcacggtgccgggctctccagagtgagggggccgtaaggagatctccaaggaagccgaaaaaagcagccagttgggcttcgggaaagacttttctgcaaaggaagtgatctggtcccagaactccagggttgaccccagtacctgacttctccgggagctgtcagctctcctctgttcttcgggcttggcgcgctcctttcataatggacagacaccagtggccttcaaaaggtctggggtgggggaacggaggaagtggccttgggtgcagaggaagagcagagctcctgccaaagctgaacgcagttagccctacccaagtgcgcgctggctcggcatatgcgctccagagccggcaggacagcccggccctgctcaccccgaggagaaatccaacagcgcagcctcctgcacctccttgccccagagac

SEQ ID NO: 7 - DNA, 325 bp, Homo sapiens
  source 1..325: mol_type = other DNA
agatcccggtgcatttaaaggccggcgtgatctgcaccacgtacctatctcggattctcagtttcacttcgctggtgtctgccaccatctttaccacatcccggtagctacatttgtctaccgcttgagccaccagcgtctgaaacctggaccggattttgcgcgccgagaggtagccggaggcggtaatgaattccacccagagggacatgctcctcttgcgcccgtcgctcaacttcagcaccgcgcagccgggcagtgagccatcgtccacgaagttgaacacccccatttggttgagataaagcaccacttcaaattcggt

SEQ ID NO: 8 - DNA, 663 bp, Homo sapiens
  source 1..663: mol_type = other DNA
actatgccttgagggtcaaaacgtctggatttcctgatcgatgctgtcgtcgctgtccacggagctactgtcgccgtcagagcgggaaggcacgttcagggagtagaagcgtgggcttgcagaaagggacctgttgctgccttacatgggggccggcagggtagtcttggaaatgcccaagattgcttccgcgcgcgtcagttcagcggacgtgtctgcctggcacgaggaccgttctacaaactcgttcctggaagccgggctcgctggaggcggagctttggtttccttcgggagcttgtggggaatggtcagcgtctaggcaccccgggcaagggtctgtggccttggtggccactggcttcctctagctgggtgttttcctgtgggtctcgcgcaaggcacttttttgtggcgctgcttgtgctgtgtgcggggtcaggcgtcctctctcctcccggcgctgggccctctggggcaggtccccgttggcctccttgcgtgtttgccgcagctagtacacctggatggcctcctcagtgccgtcgttgctgctggagtctgacgcctcgggcgcctgcgccgcacttgtgacttgctttccccttctcagggcgccagcgctcctcttgaccccgcttttattctgtggtgcttctgaag

SEQ ID NO: 9 - DNA, 1985 bp, Homo sapiens
  source 1..1985: mol_type = other DNA
gcaagtcgggtagctaccgggtgctggagaactccgcaccgcacctgctggacgtggacgcagacagcgggctcctctacaccaagcagcgcatcgaccgcgagtccctgtgccgccacaatgccaagtgccagctgtccctcgaggtgttcgccaacgacaaggagatctgcatgatcaaggtagagatccaggacatcaacgacaacgcgccctccttctcctcggaccagatcgaaatggacatctcggagaacgctgctccgggcacccgcttccccctcaccagcgcacatgaccccgacgccggcgagaatgggctccgcacctacctgctcacgcgcgacgatcacggcctctttggactggacgttaagtcccgcggcgacggcaccaagttcccagaactggtcatccagaaggctctggaccgcgagcaacagaatcaccatacgctcgtgctgactgccctggacggtggcgagcctccacgttccgccaccgtacagatcaacgtgaaggtgattgactccaacgacaacagcccggtcttcgaggcgccatcctacttggtggaactgcccgagaacgctccgctgggtacagtggtcatcgatctgaacgccaccgacgccgatgaaggtcccaatggtgaagtgctctactctttcagcagctacgtgcctgaccgcgtgcgggagctcttctccatcgaccccaagaccggcctaatccgtgtgaagggcaatctggactatgaggaaaacgggatgctggagattgacgtgcaggcccgagacctggggcctaaccctatcccagcccactgcaaagtcacggtcaagctcatcgaccgcaacgacaatgcgccgtccatcggtttcgtctccgtgcgccagggggcgctgagcgaggccgcccctcccggcaccgtcatcgccctggtgcgggtcactgaccgggactctggcaagaacggacagctgcagtgtcgggtcctaggcggaggagggacgggcggcggcgggggcctgggcgggcccgggggttccgtccccttcaagcttgaggagaactacgacaacttctacacggtggtgactgaccgcccgctggaccgcgagacacaagacgagtacaacgtgaccatcgtggcgcgggacgggggctctcctcccctcaactccaccaagtcgttcgcgatcaagattctagacgagaacgacaacccgcctcggttcaccaaagggctctacgtgcttcaggtgcacgagaacaacatcccgggagagtacctgggctctgtgctcgcccaggatcccgacctgggccagaacggcaccgtatcctactctatcctgccctcgcacatcggcgacgtgtctatctacacctatgtgtctgtgaatcccacgaacggggccatctacgccctgcgctcctttaacttcgagcagaccaaggcttttgagttcaaggtgcttgctaaggactcgggggcgcccgcgcacttggagagcaacgccacggtgagggtgacagtgctagacgtgaatgacaacgcgccagtgatcgtgctccccacgctgcagaacgacaccgcggagctgcaggtgccgcgcaacgctggcctgggctatctggtgagcactgtgcgcgccctagacagcgacttcggcgagagcgggcgtctcacctacgagatcgtggacggcaacgacgaccacctgtttgagatcgacccgtccagcggcgagatccgcacgctgcaccctttctgggaggacgtgacgcccgtggtggagctggtggtgaaggtgaccgaccacggcaagcctaccctgtccgcagtggccaagctcatcatccgctcggtgagcggatcccttcccgagggggtaccacgggtgaatggcgagcagcaccactgggacatgtcgctgccgctcatcgtgactctgagcactatctccatcatcctccta

SEQ ID NO: 10 - DNA, 213 bp, Homo sapiens
  source 1..213: mol_type = other DNA
atgcgccctctgcacccctagagccagaagacgctaggtgggctgcgcgctctgccaggcgaaggctggagcgcagacggcaaagccgcgcgtttcagccgtggtcgggtccgcaggacctgggcgtggggacaccaccaggcaggagcagaggcaggactgggacgccaaaagctgagaatcctcgatgcccgcgcgagagccccgtgttat

SEQ ID NO: 11 - DNA, 1558 bp, Homo sapiens
  source 1..1558: mol_type = other DNA
ttctggaaaccgggccccacttgcaggcccggccaccttgggttctggtggccgaagccggagctgtgtttctcgcagactcggggagctacattgtgcgtaggcaattgtttagtttgaaaggaggcacatttcaccacgcagccagcgccctgcatgcaggagaagcccccagggcccagggtcggctggctttagaggccacttaggttgttttaagcacatgtgaaagggcagacagcaggggagcaggatatgggtaagatcttcgggtctcagaacaggggctgcccttgggctgtcccggcgccctgggctctgacactgaagggtggaatggaggaaggaatggagaaaggacggtggaactttcgcttcccctctgggccgccttcccagggtcatgcctgagctgctttgatcccagtgtcgcgcatcttggtccgctacctcccaggcgatagctactgggctcctcgctggcctcactgggggccatcccgggcagtggcctgccctccgaggcccgcgggacccagcccagagctgaggttggagttctccgggccacgttccgggtcgcttaggctcggagatttcccggagaccgtcgtcctccctttctgcttggcactgcggagctccctcggcctctctcctcctctggtccctaaggcccggagtggttggcggtactggggcccgtcgtcatctctgcttctaaggcattcagactgggctccagctgggaccggcagaggaggttctcaaggaaactggtgggaaatatagttttctttcgtctggtcgtttaatttaaatgcaacttcccttggggacattttcctggacgttaaccagaccaccttgagatgtcgttgatgacctagagacccagatgatgcgtcccaggaaagttcactgctgactattgtcactcttggcgttatatctatagatatagacctatgtacatatctccaccctgatctctccgtggacatgaaacccacctaccttgtgaaagccctacgggtgacacatgactactacgtctctgtcccaacaggggctgggcctcccctgcctaatagttgccaggagtttcgcagcccaagtgaataatgtcttatggctgaacgtggccaaggactcctgtgatttaggtcccaggaggagcagagacgtccccgccccgcctgggccctgccgcattcaaagctggaagaaggcgctgatcagagaaggggcttccaggtcctgggttagaacaacaacaaacaaacgaaactccacaacagacacgcctgcccatgaccccacgcaaggacataggaagttctgtcgccttcctgctccgcggatagccgcctgccgtctgctgccaccagaacgcacggacgctcggggtggaggtagtcaatgggcagcaggggacccccagcccccacaagcgcggctccgaggacctggaagcgggtgcctgtcgctctccgcaggctccgctctgcctccaggagcaagatccccaaaagggtctggaagctgtggagaaaac

SEQ ID NO: 12 - DNA, 1264 bp, Homo sapiens
  source 1..1264: mol_type = other DNA
ttttttaaacacttcttttccttctcttcctcgttttgattgcaccgtttccatctgggggctagaggagcaaggcagcagccttcccagccagcccttgttggcttgccatcgtccatctggcttataaaagtttgctgagcgcagtccagagggctgcgctgctcgtcccctcggctggcagaagggggtgacgctgggcagcggcgaggagcgcgccgctgcctctggcgggctttcggcttgaggggcaaggtgaagagcgcaccggccgtggggtttaccgagctggatttgtatgttgcaccatgccttcttggatcggggctgtgattcttcccctcttggggctgctgctctccctccccgccggggcggatgtgaaggctcggagctgcggagaggtccgccaggcgtacggtgccaagggattcagcctggcggacatcccctaccaggagatcgcaggtaagcgcgggcgcgctgcaggggcaggctgcagccctcggctgccgcacgtcccactggccgcccggcgtccccttccttccccctgttgctgagttggtgctcactttctgccaccgctatgggactccgcgtctccgtgttgggcggcggatgctcctgcggcttcttcggcgggggaaggtgtgcgtctccgccgcctcattgtgtgcacacgcgggagcaccctggctcccgcctcccgctgctctcgcgcccttctaccccttagttgatggctcaggcccggctggccagggagcccgggtcactccggggcggctgcaaggcgcagacggagagccgagccgggcgctcactccgcgttctggttcgggcaaacttggaagaactgcgaccgcagtttgcccagcgccacagtctgagtggcgccttctccactcccgcccttgcgccggcaggggcggtggagagacgcggagggctcccccagcccctctctcccctatccgtccttcgggcgacagagcgcccggcgctcgggccgggggcgggcaaggctgggagggaccctcgccggggacctggcctctggacgccggcgtttcaaggctggtttggggacttcacgggctgcctgtttcagatgtggggcgggctttcccgttagggttcctcagtgcttccccagttgctgttggccactcagggcccggggacaccctgccacccggtctggagccggcctcgtctgccagcgaacagccaactttagcgggtggctcagctggggatt

SEQ ID NO: 13 - DNA, 761 bp, Homo sapiens
  source 1..761: mol_type = other DNA
cactcagtgtgtgcatatgagagcggagagacagcgacctggaggccatgggtgggggcgggtggtgaagctgccgaagcctacacatacacttagctttgacacttctcgtaggttccaaagacgaagacacggtggcttcagggagacaagtcgcaagggcgacttttccaagcgggagatggtgaagtctttggacgtgtagtgggtaggtgatgatccccgcagccgcctgtaggcccgcagacttcagaaaacaagggccttctgtgagcgctgtgtcctccccggaatccgcggcttaacacattctttccagctgcggggccaggatctccaccccgcgcatccgtggacacacttagggtcgcctttgttttgcgcagtgattcaagttgggtaacccttgctcaacacttgggaaatggggagaatctcccccacccgcaacctcccgcaccccaggttcccaaaatctgaatctgtatcctagagtggaggcagcgtctagaaagcaaagaaacggtgtccaaagaccccggagagttgagtgagcgcagatccgtgacgcctgcggtacgctagggcatccaggctagggtgtgtgtgtgcgggtcggggggcgcacagagaccgcgctggtttaggtggacccgcagtcccgcccgcatctggaacgagctgcttcgcagttccggctcccggcgccccagagaagttcggggagcggtgagcctagccgccgcgcgctcatgtttatt

SEQ ID NO: 14 - DNA, 1198 bp, Homo sapiens
  source 1..1198: mol_type = other DNA
agtcactccaggatcagaggccgcgtcggttctgcttggggcatgggcagagggaggctgctggggccaagccccggctggacgcgagggaagaaactcgtcccaggacccgcacgcccatacctggctgtcccagagctcttccctaggccggcaccttcgctcttcctcttccccaccccctagcccttttgtctctttttcagacggatgttttcagtctcaagtggttttattttccgcacaaaaccctgagatcaagggcagatcacagactgtaccggaggctcgggtttccctggactctgtgctgttctgcgtcccagggttggctaggaaggaaggcctgggccggcgaggtgacgggtctcccgcccaggtcggcaggacggggggaggtgtgtcccggtaggtccctggtgagctcacccgtggcatcggggacccgcgggaacccaccgggcgcccactagagactcgggtcctaccctcccccacactactccaccgaaatgatcggaagggcgcgctaggcctgcttccaagggctcagtgataaaggcctcaaaatcacactccatcaagacttggttgaagctttgggtaggtttgttgttgttgttgttgttgtttgtttgtttgttttagcagacacgtcctggaaagaggtcctcagaacccaaaggttcaataatgatttgtggatggattgattatagtctgatatcgctctggttccacagaaacccggagctccttggcccactgttaccccagcagacctaaatggacggtttctgtttttcactggcagctcagaactggaccggaagaagttcccctccacttcccccctcccgacaccagatcattgctgggtttttattttcgggggaaaaacaacaacaacaacaacaaaaaaaacactaggtccttccagactggatcaggtgatcgggcaaaaaccctcaggctagtccggctgggtgcccgagcatgaaaaggcctccgtggccgtttgaacagggtgttgcaaatgagaacttttgtaagccataaccagggcatcctgagggtctgagttcacggtcaaggctgtgggctactaggtccagcgagtccaggcctcgccccgcccccgagctgccacagccaagatcttcggcagggaattcgagaccagggtcctcccactcct

SEQ ID NO: 15 - DNA, 377 bp, Homo sapiens
  source 1..377: mol_type = other DNA
tttcgtgccgctgttttcaatgcgctaacgaggcacgttattcttagccgcgtccgggaggggatcacattcctgcgcagttgcgctgctggcggaagtgacttgttttctaacgaccctcgtgacagccagagaatgtccgtttctcggagcgcagcacagcctgtcccatcgagaagcctcgggtgaggggcccggtgggcgcccggaggccgctggagggctgtgggagggacggtggctccccactcccgtggcgaagggcaggcaaaccagaagcctcttttgagagccgtttgggattgagacgagtaagccacagcgagtggttagaagtaggttaggaagaaggggaggtaagaaagccgagtagggtt

SEQ ID NO: 16 - DNA, 256 bp, Homo sapiens
  source 1..256: mol_type = other DNA
gttcggtggacaagggggcagcgcccacagcaagccggaaagagggaggcgcggggccgcgcttggggcctgccgctgcacgccagcctgggcaaagagctgccaccttctgcgggcgaagcgggtcgggacgcaggacggcagcggggctggaggcagctacgtgggtccacacccccatgccctgcaaggctccttggccctgcttctcctctgtctcggcgggagaggagcagcctcggttttacagaatttc

SEQ ID NO: 17 - DNA, 189 bp, Homo sapiens
  source 1..189: mol_type = other DNA
tgtgccatttagtgagaggtgttttgggcaaagaatcaatttaactgtgactgaccgacgggcttgactgtattaattctgctaccgaaaaaaaaaaaaaaaaaaaagcaatgagccgcaagccttggactcgcagagctgccggtgcccgtccgagagccccaccagcgcggctcacgcctcagtctc

SEQ ID NO: 18 - DNA, 707 bp, Homo sapiens
  source 1..707: mol_type = other DNA
agagtcccagttctgcaggccgctccagggctaggggtagagatggtggcaggtggtgcgtcaactctctagggaagaggaacttgcattacaaagacttgtctttctgagctgaagtcaaaacgggggcgtcaagcgcgctccgtttggcggcggtggaggggccgcgcgcccgcgctgtcccagccggagctgccctggctggtgattggaggtttaacgtccggaattcaggcgcttctgcagctcagatttgccggccaaggggcctcagttgcaacttttcaaaatggtgtttctggaaaataacaaattcagactcaactggtgacagcttttggctatagagaatgaaactgcttccctttggcggtggaactcttaaacttcgaagagtgaaagaatacaatgaaataaaatgccataagatcactggatttttcagaaaaaggaagaccccaaattactcccaaaatgaggctttgtaaattcttgttaaaaatctttaaatctcgaatttccccctacaacatctgatgagtgctttaagagcaaacgagcaaatcccacctcgagaatcaacaaacccaagctctggccaaggctctccccgcgttttcttctcgtgacctggggaatgtcccgccccatcgctcacctggctcttgtcatctcgctcatcttgaagtgacccgtggacaatgctg

SEQ ID NO: 19 - DNA, 182 bp, Homo sapiens
  source 1..182: mol_type = other DNA
agctgccctctgtggccatgagcgggtgtccagccccttccaaggctgcaccggggagacgctggttttctgctcgctgtgaccgaacaaagcccctaagagtcagtgcgcggaacagaagagccggaccccgacgggccgagtcccaacgtgaggcacccggcagagaaaacacgttcacg

SEQ ID NO: 20 - DNA, 179 bp, Homo sapiens
  source 1..179: mol_type = other DNA
cctcggcagcaccggcatggctggaggccagtacggccaggtgtggcgggagggagcgccgtctggcttgggtcgtccatcctgacaggacgctgcaagggcaggagccccgcgccccgtgtcctgcgcccccgctcgaggacaagccccagccgccggtctccgctgggttccgacag

SEQ ID NO: 21 - DNA, 369 bp, Homo sapiens
  source 1..369: mol_type = other DNA
ctttaagaggctgtgcaggcagacagacctccaggcccgctaggggatccgcgccatggaggccgcccgggactatgcaggagccctcatcaggcgagtgccccgcgtccccctgattgccgtgcgcttccaatcgccttgcgttcggtggcctcatattcccctgtgcgcctctagtaccgtaccccgctcccttcagccccctgctccccgcattctcttgcgctccgcgaccccgcgcacacacccatccgccccactggtgcccaagccgtccagccgcgcccgcgggcagagcccaatcccgtcccgcgcctcctcaccctcttgcagctgggcacaggtaccaggtgtggctcttgcgaggtg

SEQ ID NO: 22 - DNA, 176 bp, Homo sapiens
  source 1..176: mol_type = other DNA
agacttgcagaactcgggccccctggaggagacctaaccgccacggtcttggggaggttccggagggcctcggttgtctgcactcccaacaccaagaaacccctgagacgcgaagctgccagcgtgctgccctcagagcagggcgacgcaaagccagcggaccccggggtggcggg

SEQ ID NO: 23 - DNA, 167 bp, Homo sapiens
  source 1..167: mol_type = other DNA
tgctcggctggggggctcgctccgcactttcggtgccagaaaatgcccagaggagcggggcggccccagagcctcctttcggggcgcgaggcccggcgcgtgtgtacggagtccagtccccccagggagtggggtgcccgcaccttcccctccgcgctcggagccac

SEQ ID NO: 24 - DNA, 1205 bp, Homo sapiens
  source 1..1205: mol_type = other DNA
tcttgcacacctgcttgtagttctgcaccgagatctggtcgttgaggaactgcacgcagagcttggtgacctgggggatgtgcaggatcttgctgaccgacagcacctcctccaccgtgtccagggacagggtcacgttggccgtgtagaggtactcgagcaccaggcgcagcccgatggacgagcagccctgcagcaccaggttgttgatggcccgggggctggtcagcagcttgtcgtcgggggaggaagaaggagtcccgggctcctcctgcggcggcggctgctgctgctgtgacggctgctgctgcggcggctgctgctggtccttgggggcccccaggccgtcctggccgccgacccctcccccgagaggggggtggctggagaagagcgatcggaagtactgcgagcaggaggccagcacggccttgtggcaatggaactgctggccctgggccgtcagggtcacgtcgcaaaacagctgcttcctccacagcaggttgaggccgtgcagcaggttgtcgctgtggctggggtcgaaggtggaggtcctgtccccggatctggacatggcgagctgactcggtgcacctggctttaaaccctcctccaacctggcagacaggggtgggggatgggagggaggggagcagggtggtggagcgggtggggtgtggtcggggtggggaagggtgtggaggggaggggagggcgaagaacaagaatcaaggctcagcttgactccctcctggcgcgctccggaccccgaccctaggaggaaagtccgaagacgctggatccgtgagcgccaccagaagggccctgtctggggtcccggcgccggttctgcgccctgcggctcctctcgccacctcccacacacttcgtccctcactttcctaaaaccaaccacctcagctcggctgttggcagcaacagcagtggcagcagcgacggcaaagtggcggctgaggccgaggcacctcgtgggctcgtgtccatgccgggccagatgaagggaaaggccgggaagtggggagccgggggtgccctgaaagctcagaggcgaccgacggcgaaggttccaggtcaacttgtgcccgaagctttgcttttcgcagttggcccagtttgggggagggggtaggaacaggggcccgaccagcgtgcggggtgtgcgaatcttagctctccaaaagctg

SEQ ID NO: 25 - DNA, 44 bp, Homo sapiens
  source 1..44: mol_type = other DNA
cctctgtgttagtgccctcgggaatttggttgatggggtgtttg

SEQ ID NO: 26 - DNA, 5002 bp, Homo sapiens
  source 1..5002: mol_type = other DNA
tgatgtcgcacctgaacggcctgcaccacccgggccacactcagtctcacgggccggtgctggcacccagtcgcgagcggccaccctcgtcctcatcgggctcgcaggtggccacgtcgggccagctggaagaaatcaacaccaaagaggtggcccagcgcatcacagcggagctgaagcgctacagtatcccccaggcgatctttgcgcagagggtgctgtgccggtctcaggggactctctccgacctgctccggaatccaaaaccgtggagtaaactcaaatctggcagggagaccttccgcaggatgtggaagtggcttcaggagcccgagttccagcgcatgtccgccttacgcctggcaggtaaggccggggctagccaggggccaggctgctgggaagagggctccgggtccggtgcttgtggcccaagtctgcgcgccgagtcacttctcttgattctttccttctctttcctatacacgtcctctttcttctcgtttttatttcttcttccattttctctttctcttccgctcttcccctactttcccttctcccttttctttttctttcttactctctccttgtccctgagctttcattgaccgacccccccccatttcattcgccctcccctcaatgtgccaacctttgccctatttccgatcttcccaggtactgggaggcgggatgggggtgtgcgttttcctctaggagccctgtctttccaagacccacagaaaccaggacctgcccttattcaaaaccccatgcacttcaagtctcttttagacaacacatttcaattttccgggctgactagtctccctgtgcagaggcagttgagaggctttgctctgcagagggaaaagagctctctactctcccacccaccatataggcaaacttatttggtcattggctgaaggcacagccttgcccccgcggggaaccggcggccaggatacaacagcgctcctggagcccatctctggccttggcgttggcgcagggactttctgaccgggcttgaggggctcgggccagctccaatgtcactacctacagcgagggcagggtgtaaggttgagaaggtcacattcaccgctttgggaggacgtgggagaagagactgaggtggaaagcgctttgccttgctcaccggccgtccttgccccggtcccagcgtttgctgggatttgccaggatttgccggggctccgggagaccctgagcactcgcaggaagaggtgctgagaaattaaaaattcaggttagttaatgcatccctgccgccggctgcaggctccgcctttgcattaagcgggcgctgattgtgcgcgcctggcgaccgcggggaggactggcggcccgcgggaggggacgggtagaggcgcgggttacattgttctggagccggctcggctctttgtgcctcctctagcggccaagctgcgaggtacagccctctattgttctaggagcacagaaacctcctgtgtgggcggcgggtgcgcgagctagagggaaagatgcagtagttactgcgactggcacgcagttgcgcgcttttgtgcgcacggaccccgcgcggtgtgcgtggcgactgcgctgcccctaggagcaagccacgggcccagaggggcaaaatgtccaggtcccccgctgggaaggacacactataccctatggcaagccagggtgggcgacttcccatggatcgggtggaggggggtatctttcaggatcggcgggcggtctaggggaacaattcgtggtggcgatgatttgcatagcgcgggtcttgggatgcgcgcggttccgagccagcctcgcacagctcgcttccggagctgcgagctcaggtttccacccccgatcccccgggctttcctcgcaccgctgagcccagcttgtggggtgcactcgaccaacgcccgacagggctggggaatgtgacaggcagcaggttcacccgggcttggggagggggagtttccgctttgacagcattttcctttgccgtctgctggtggattcctattcccagtcggtaatcgccccgcagtgttgatctaagaaggtaaagaaaactaggtttccctgcaaagagcctcccccaaatcggcggactccggatactttgagtggatttagaaatttatgtaatctttctcctttagtttatttttcatcctctcctacagttttctctgatttgctgttggttcggggcaagataaagcagccagtagagagcgataataatagcggcgggaaatgaactggagactggctgacagttcttaacattttgtcatagatccccccgaatgtcccaggctgtctctggtgggttttagtacccgccggcttcttgggcaccggggaccagaaggaacttggcagctggtcttaggggtacagttaaaggcaggatgacagctattctcctgctcatctcagagcgctgccgccccctcatgccggtcgcgcaaagaacacagcttttaaaaaacacgtgccttctgcccatataggtctgaaagtgatgaggaaagtaatgcttcgcctattagcgagtttcagcttttaaaatgatcccaagcgttgctgagatgagaaagcgtggcatcccgggggtcctcagccccacccgcgcccatggtgcaagtctgcagggacaggcccgggacagcactgcccacgctgctagattttccgcagaggatcgctgaagctgccttcgtgggagacagaatgcctcctccagcgagtggaaaaggcctgctgaggaccccgctttgctcgagcattcaaatgtgtgtctgttttattaccctgggttgaaaagggacaagagctttagcctttttatctggccattttatcagcaactacaagtgtgttgagtggttattattacataggaggcttttcagtttggggtcagtagatcagtctcttcagacactgatgcagaagctgggactggtaagtaggtattatgtgctcggagcgctaggggacaggagcaaatggagaagaaaagcggaggctttctccgcccggagtatcgatcggaatccccgccggtacgccgcagagggccctcgccgttgggccccgggggtttaacaagcccagccgctccgcaggcggctcggccggactctcagaccggtgcctggaagacaccgtccctgcccccctcccgccaaacctgcctcttctctttctctcataggttataggttccctttctctctcattttggccccgcccccgggtcctgccaaacagccaagcaggccggggtttagggggctcagaatgaagaggtctgatttggccagcgccggcaaagctcacccttaggcgaggtcacaacagaggcaggtccttcctgcccagcctgccggtgtagtcacagccaagggtggcacttgaaaggaaaagggagaaaacttcggagaaatttagattgccccaacgttagatttcagagaaattgactccaaatgcacggattcgttcggaaagggcggctaagtggcaggtggttgcaaccccgcccggtcgggccttcgcagaggttccccaagaccagcccttgcagggcggttttcagcaacctgacaagaggcggccaagacaaatttctgcgggttcgagcacacactctcgggcgttgggccccagagacctctaaaccaagcacaaacaagaagggagtgagagaacccaggctagaacttgcacgggcatcccactgaggaaaagcgaggcctcggtggcaggcatgttttcttccgacgcccgaaaatcgagccgagcgcccgactacatttactgcagaggtttccgcctccagtgagcccggatcccccagcggcctgcccggagctggtctccagtccccgccgtagtccgacgcacggccctctcctggcagcaagctcccagcggccagtctgaagccaattctgttcaggcggccgagggcccttagccaacccaccatgatgtcgcctgggccacctgatgcccgcagcggcgggacacggcccgggcagtgcgcagtggctcctgctaggggcaccgcgtgcgtgcttgtctcccgctgcgccggggacgtccttgggtgacacgggccgctgggcacctcccaagccgaggaaacggacccccttcgcagagtctcgcgcccaccccccaacctcccacctcgtttctcgctgctagggctcccgactcagcccacctctcctggcggtttagttagggatcagagctggagaggctgaacgcaacccgtgccagtacggaacagacgatatgtttgcctgctagctgcttggatgaataattgaaaagttcgctgcagtctgtgcttcgtcaagtcccgggtgccgggagaacaccttcccaacacgcatcagggtgggcgggagcgggcagaggaggcgggacccgagggaggagagtgaacccgagcaggagaagcagcccaggcagccaggcgccctcgatgcgagaggctgggcatttatttttattccaggctttccactgtgtggttatgtcactttctcaaacaaatgtgtatatggagggagatcgatgctgataatgtttagaagattaaaagagcattaatgctggcaacaataacgtaaacgtgtggacccagatttcattgatctggaacttgatccggcgcgtttccagtaagcccgacggcgcgctcttcccagcagagcgctcaccagcgccacggccccgcggttttccagcggtgccgcttcgccagctctgcgcgggttctcccgtctgaccgcagctcctcccccgcgaggccccagcccgccttacttccccgaggttttctcctcctctcgcggggctctctgccctctgcaccccctcccccgacctctgcaccacccgcccctgtgcgcacacaccgctacttgcgcttccggcgatccgcctg

SEQ ID NO: 27 - DNA, 150 bp, Homo sapiens
  source 1..150: mol_type = other DNA
aaccggagatctgcttggtgaactgagaggagtccttaggagagcggggacgccaggggccgggggacacttcgctctcgccctagggaaggtggtcttgacgctttctattgaagtcaaacttgaaaatatcagctgccgctggactat

SEQ ID NO: 28 - DNA, 339 bp, Homo sapiens
  source 1..339: mol_type = other DNA
cgtgagcagaacgcccgccctggagcagttaggaccgaaggtctccggagagtcgccggcggtgccaggtaacgcagagggctcgggtcgggccccgcttctggggcttgggactccgggcgcgcggagccagccctctggggcgaaatccccgggcggcgtgcgcggtccctctccgcgctgtgctctcccagcaactccctgccacctcgacgagcctaccggccgctccgagttcgacttcctcggacttagtgggagaaggggttggaaatgggctgccgggactgggggagctgctctctggaagcagggaagctggggcgcaccggggcaggt

SEQ ID NO: 29 - DNA, 1961 bp, Homo sapiens
  source 1..1961: mol_type = other DNA
tagaagaggaagactcctctggccccactaggtatcatccgcgctctcccgctttccacctgcgccctcgcttgggccaatctctgccgcacgtgtccatccctgaactgcacgctatcctccacccccggggggttcctgcgcactgaaagaccgttctccggcaggttttgggatccggcgacggctgaccgcgcgccgcccccacgcccggttccacgatgctgcaatacagaaagtttacgtcggccccgacccgcgcgggactgcagggtccgccggagcgcggcgcagaggcttttcctgcgcgttcggccccgggaaaggggcgggagggctggctccgggagcgcacgggcgcggcggggagggtactcactgtgaagcacgctgcgcccatggatcatgtctgtgcgttacaccagaggctccgggctccactaattccatttagagacgggaagacttccagtggcggggggaggacagggtcgagaggtgttaaagacgcaaagcaagaaggaaataaaggggggccgagagggagaccgagaggaagggggagctccgagcccacgctgcagccagatccggatgagtccgtcctccgccccgggcgggctctcgctctcgctggccctcagcgccgcgcagccagcagcatccccaccgtgacgctcgcatcacacccgggcgccggccgccaccatccgcgccgccgccgtcaggaccctcctcccgggcatcgtcgccgccgcggggtcgggaggacgcggcgcgcgggaggcggcggtcgcagggcgagccccgggacgccccgagccggggccggggccggggagagggcgcagcgaggtgggggccagtccagaccgacggcagcgacggagcgggcggcggcggcggcgccggcggcggcggggtggctcagtccccagtctcagacgcgccgcgcagcaggtcggagcagcctccccgggaggatgtccagcggcagcgctcctcgctccagcccttggggatcttccgctgaggcattgaaggcaggaagaaggggtccgtcatcggctcgccgggctgcgcgccacctctgctatcttgcggaaagaggagcgggtgggtgggcgtctgggaggcgggctggagggcggtgcaggggagcggggcggccggggggggggccggggggcggggaagggagggaggagaaaggagccggaagagggcagagttaccaaatgggctccttagtcatggcttggggctccacgaccctcctggaagcccggagcctgggtgggatagcgaggctgcgcgcggccggcgccccggggctggtgcgcggcagaatggggccgcggcggcggcagcaaggacatcccagccgcgcggatctgggggaggggcggggagggggtgaggacccggctgggatccgcggctcggcccgccagggcgcagagagaggatgcagccgcaaatcccgagccggatcctcgtgccggacggaaggcgtggaagcgggaggggccttcgtgtgaaaatcccttgtggggtttggtgtttcactttttaaaggttagaccttgcgggctctctgcctcccaccccttcttttccatccgcgtaaaggaactgggcgccccctctccctccctccctggggcgcaggtttcgccgcggactccgcgctcagcttgggagacacggcaggggcgcgccccagggaaaggcggccgtaaaagtttcgcggttgagcactgggcctgatgtccagtccccccaccaaattactcctgcaaagacgcgggcttcttgcaattgagccccccacctcgaggtatttaaaaccaccccaaggcacacacggacccccgttcccccgcgccacttcctcctacaggctcgcgcggcgcgttaaagtctgggagacacgagttgcggggaaacagcaccggaag

SEQ ID NO: 30 - DNA, 314 bp, Homo sapiens
  source 1..314: mol_type = other DNA
aagaaacagctcatttcggagctgaggacaaggcgtgggaagaagacgcgtttggtttcacccaggcgggtggcggcaaagctgtgggatgcgcgctgcacactccttccgtcatcccgttcccaccttccacacacacctgcgggaggtcggacatgtcctgattgcgtgttcatcacgatggcaaaccgaacatgaggagaacgccactgacgctgggtgcgccggctttcccagccctcgtgcataacggggagggagatgcagaagttttttccaacatcggtgcaaaggggaagctgaggttttcctat

SEQ ID NO: 31 - DNA, 584 bp, Homo sapiens
  source 1..584: mol_type = other DNA
tctgtcagctgctgccatggggcagcgggaaggccctggagggtgcctgggctgtgtctggtcccggccacgcgtccctgcagcgtctgagaccttgtggaacacacttgacccggcgctgggacggggtcggcccacacgcaccgccagcccgcaggagtgaggtgcaggctgccgctggctccttaggcctcgacagctctcttgaggtcggccctcctcccctcccgagagctcagcagccgcagacccaggcagagagagcaaaggaggctgtggtggcccccgacgggaacctgggtggccgggggacacaccgaggaactttccgccccccgacgggctctcccaccgaggctcaggtgctcgtgggcagcaaggggaagccccatggccatgccgcttccctttcaccctcagcgacgcgccctcctgtgcccgcggggaacaagacggctctcggcggccatgcaggcggcctgtcccacgaacacgatggagacctcagacgccgtccccaccctgtcactgtcaccatcacccatcctgtcccctcacgcctccccacatcccatcattactac

SEQ ID NO: 32 - DNA, 349 bp, Homo sapiens
  source 1..349: mol_type = other DNA
gaagtagaatcacagtaaatgaggagttagggaatttagggtagagattaaagtaatgaacagaggaggaggcctgagacagctgcagagagaccctgtgttccctgtgaggtgaagcgtctgctgtcaaagccggttggcgctgagaagaggtaccgggggcagcacccgcctcctgggagagggatgggcctgcgggcacctgggggaaccgcacggacacagacgacactataaacgcgggcgagacatcagggaccgggaaacagaaggacgcgcgtttcgagcagctgcccagtgggccacaagccccgccacgccacagcctcttcccctcagcacgcagaga

SEQ ID NO: 33 - DNA, 3510 bp, Homo sapiens
  source 1..3510: mol_type = other DNA
tactccggcgacgggaggatgttgagggaagcctgccaggtgaagaaggggccagcagcagcacagagcttccgactttgccttccaggctctagactcgcgccatgccaagacgggcccctcgactttcacccctgactcccaactccagccactggaccgagcgcgcaaagaacctgagaccgcttgctctcaccgccgcaagtcggtcgcaggacagacaccagtgggcagcaacaaaaaaagaaaccgggttccgggacacgtgccggcggctggactaacctcagcggctgcaaccaaggagcgcgcacgttgcgcctgctggtgtttattagctacactggcaggcgcacaactccgcgccccgactggtggccccacagcgcgcaccacacatggcctcgctgctgttggcggggtaggcccgaaggaggcatctacaaatgcccgagccctttctgatccccacccccccgctccctgcgtcgtccgagtgacagattctactaattgaacggttatgggtcatccttgtaaccgttggacgacataacaccacgcttcagttcttcatgttttaaatacatatttaacggatggctgcagagccagctgggaaacacgcggattgaaaaataatgctccagaaggcacgagactggggcgaaggcgagagcgggctgggcttctagcggagaccgcagagggagacatatctcagaactaggggcaataacgtgggtttctctttgtatttgtttattttgtaactttgctacttgaagaccaattatttactatgctaatttgtttgcttgtttttaaaaccgtacttgcacagtaaaagttccccaacaacggaagtaacccgacgttcctcacactccctaggagactgtgtgcgtgtgtgcccgcgcgtgcgctcacagtgtcaagtgctagcatccgagatctgcagaaacaaatgtctgaattcgaaatgtatgggtgtgagaaattcagctcggggaagagattagggactgggggagacaggtggctgcctgtactataaggaaccgccaacgccagcatctgtagtccaagcagggctgctctgtaaaggcttagcaattttttctgtaggcttgctgcacacggtctctggcttttcccatctgtaaaatgggtgaatgcatccgtacctcagctacctccgtgaggtgcttctccagttcgggcttaattcctcatcgtcaagagttttcaggtttcagagccagcctgcaatcggtaaaacatgtcccaacgcggtcgcgagtggttccatctcgctgtctggcccacagcgtggagaagccttgcccaggcctgaaacttctctttgcagttccagaaagcaggcgactgggacggaaggctctttgctaaccttttacagcggagccctgcttggactacagatgccagcgttgcccctgccccaaggcgtgtggtgatcacaaagacgacactgaaaatacttactatcatccggctcccctgctaataaatggaggggtgtttaactacaggcacgaccctgcccttgtgctagcgcggttaccgtgcggaaataactcgtccctgtacccacaccatcctcaacctaaaggagagttgtgaattctttcaaaacactcttctggagtccgtcccctccctccttgcccgccctctacccctcaagtccctgcccccagctgggggcgctaccggctgccgtcggagctgcagccacggccatctcctagacgcgcgagtagagcaccaagatagtggggactttgtgcctgggcatcgtttacatttggggcgccaaatgcccacgtgttgatgaaaccagtgagatgggaacaggcggcgggaaaccagacagaggaagagctagggaggagaccccagccccggatcctgggtcgccagggttttccgcgcgcatcccaaaaggtgcggctgcgtggggcatcaggttagtttgttagactctgcagagtctccaaaccatcccatcccccaacctgactctgtggtggccgtattttttacagaaatttgaccacgttccctttctcccttggtcccaagcgcgctcagccctccctccatcccccttgagccgcccttctcctccccctcgcctcctcgggtccctcctccagtccctccccaagaatctcccggccacgggcgcccattggttgtgcgcagggaggaggcgtgtgcccggcctggcgagtttcattgagcggaattagcccggatgacatcagcttcccagccccccggcgggcccagctcattggcgaggcagcccctccaggacacgcacattgttccccgcccccgcccccgccaccgctgccgccgtcgccgctgccaccgggctataaaaaccggccgagcccctaaaggtgcggatgcttattatagatcgacgcgacaccagcgcccggtgccaggttctcccctgaggcttttcggagcgagctcctcaaatcgcatccagagtaagtgtccccgccccacagcagccgcagcctagatcccagggacagactctcctcaactcggctgtgacccagaatgctccgatacagggggtctggatccctactctgcgggccatttctccagagcgactttgctcttctgtcctccccacactcaccgctgcatctccctcaccaaaagcgagaagtcggagcgacaacagctctttctgcccaagccccagtcagctggtgagctccccgtggtctccagatgcagcacatggactctgggccccgcgccggctctgggtgcatgtgcgtgtgcgtgtgtttgctgcgtggtgtcgatggagataaggtggatccgtttgaggaaccaaatcattagttctctatctagatctccattctccccaaagaaaggccctcacttcccactcgtttattccagcccgggggctcagttttcccacacctaactgaaagcccgaagcctctagaatgccacccgcaccccgagggtcaccaacgctccctgaaataacctgttgcatgagagcagaggggagatagagagagcttaattataggtacccgcgtgcagctaaaaggagggccagagatagtagcgagggggacgaggagccacgggccacctgtgccgggaccccgcgctgtggtactgcggtgcaggcgggagcagcttttctgtctctcactgactcactctctctctctctccctctctctctctctcattctctctcttttctcctcctctcctggaagttttcgggtccgagggaaggaggaccctgcgaaagctgcgacgactatcttcccctggggccatggactcggacgccagcctggtgtccagccgcccgtcgtcgccagagcccgatgacctttttctgccggcccggagtaagggcagcagcggcagcgccttcactgggggcaccgtgtcctcgtccaccccgagtgactgccc

SEQ ID NO: 34 - DNA, 333 bp, Homo sapiens
  source 1..333: mol_type = other DNA
ttaattcgaaaatggcagacagagctgagcgctgccgttcttttcaggattgaaaatgtgccagtgggccaggggcgctgggacccgcggtgcggaagactcggaacaggaagaaatagtggcgcgctgggtgggctgccccgccgcccacgccggttgccgctggtgacagtggctgcccggccaggcacctccgagcagcaggtctgagcgtttttggcgtcccaagcgttccgggccgcgtcttccagagcctctgctcccagcggggtcgctgcggcctggcccgaaggatttgactctttgctgggaggcgcgctgctcagggttctg

SEQ ID NO: 35 - DNA, 385 bp, Homo sapiens
  source 1..385: mol_type = other DNA
ccggtccccagtttggaaaaaggcgcaagaagcgggcttttcagggaccccggggagaacacgagggctccgacgcgggagaaggattgaagcgtgcagaggcgccccaaattgcgacaatttactgggatccttttgtggggaaaggaggcttagaggctcaagctataggctgtcctagagcaactaggcgagaacctggccccaaactccctccttacgccctggcacaggttcccggcgactggtgttcccaagggagccccctgagcctaccgcccttgcagggggtcgtgctgcggcttctgggtcataaacgccgaggtcgggggtggcggagctgtagaggctgcccgcgcagaaagctccaggatcccaatatgtg

SEQ ID NO: 36 - DNA, 105 bp, Homo sapiens
  source 1..105: mol_type = other DNA
gcgcaggtccccccagtccccgagggagtgcgcccgacggaaacgcccctagcccgcgggcctcgctttcctctcccgggttcctgggtcacttcccgctgtctc

SEQ ID NO: 37 - DNA, 147 bp, Homo sapiens
  source 1..147: mol_type = other DNA
ttccctcgcggctttggaaagggggtgcaaatgcacccttctgcgggcccgctacccgctgcaacacctgtgtttcctttctgggcaccttctaggtttctagatattgctgtgaatacggtcctccgctgtacagttgaaaacaaa

SEQ ID NO: 38 - DNA, 365 bp, Homo sapiens
  source 1..365: mol_type = other DNA
tgggaatttaggtcgggcactgccgatatgtcgccttccacaaggcgggcccgggcctctgctgaccgtgcaccggtcctggggctgggtaattctgcagcagcagcgcagcccatgccggggaatttgcgggcagaggagacagtgaggcccgcgttctgtgcgggaactcccgagctcacagagcccaagaccacacggctgcatctgcttggctgactgggccaggcccacgcgtagtaacccggacgtctctctctcacagtccccttgcgtctggccagggagctgccaggctgcaccccgcggtggggatcgggagaggggcagtgtcgcccatccccggaaggctgagcctggtgcag

SEQ ID NO: 39 - DNA, 418 bp, Homo sapiens
  source 1..418: mol_type = other DNA
cggttttctcctggaggactgtgttcagacagatactggtttccttatccgcaggtgtgcgcggcgctcgcaagtggtcagcataacgccgggcgaattcggaaagcccgtgcgtccgtggacgacccacttggaaggagttgggagaagtccttgttcccacgcgcggacgcttccctccgtgtgtccttcgagccacaaaaagcccagaccctaacccgctcctttctcccgccgcgtccatgcagaactccgccgttcctgggaggggaagcccgcgaggcgtcgggagaggcacgtcctccgtgagcaaagagctcctccgagcgcgcggcggggacgctgggccgacaggggaccgcgggggcagggcggagaggacccgccctcgagtcggcccagccctaacactcaggac

SEQ ID NO: 40 - DNA, 906 bp, Homo sapiens
  source 1..906: mol_type = other DNA
agggaatcgggctgaccagtcctaaggtcccacgctcccctgacctcagggcccagagcctcgcattaccccgagcagtgcgttggttactctccctggaaagccgcccccgccggggcaagtgggagttgctgcactgcggtctttggaggcctaggtcgcccagagtaggcggagccctgtatccctcctggagccggcctgcggtgaggtcggtacccagtacttagggagggaggacgcgcttggtgctcagggtaggctgggccgctgctagctcttgatttagtctcatgtccgcctttgtgccggcctctccgatttgtgggtccttccaagaaagagtcctctagggcagctagggtcgtctcttgggtctggcgaggcggcaggccttcttcggacctatccccagaggtgtaacggagactttctccactgcagggcggcctggggcgggcatctgccaggcgagggagctgccctgccgccgagattgtggggaaacggcgtggaagacaccccatcggagggcacccaatctgcctctgcactcgattccatcctgcaacccaggagaaaccatttccgagttccagccgcagaggcacccgcggagttgccaaaagagactcccgcgaggtcgctcggaaccttgaccctgacacctggacgcgaggtctttcaggaccagtctcggctcggtagcctggtccccgaccaccgcgaccaggagttccttcttcccttcctgctcaccagccggccgccggcagcggctccaggaaggagcaccaacccgcgctgggggcggaggttcaggcggcaggaatggagaggctgatcctcctctagccccggcgcattcacttaggtgcgggagccctgaggttcagcctgactttc

SEQ ID NO: 41 - DNA, 860 bp, Homo sapiens
  source 1..860: mol_type = other DNA
cactacggatctgcctggactggttcagatgcgtcgtttaaaggggggggctggcactccagagaggagggggcgctgcaggttaattgatagccacggaagcacctaggcgccccatgcgcggagccggagccgccagctcagtctgacccctgtcttttctctcctcttccctctcccacccctcactccgggaaagcgagggccgaggtaggggcagatagatcaccagacaggcggagaaggacaggagtacagatggagggaccaggacacagaatgcaaaagactggcaggtgagaagaagggagaaacagagggagagagaaagggagaaacagagcagaggcggccgccggcccggccgccctgagtccgatttccctccttccctgacccttcagtttcactgcaaatccacagaagcaggtttgcgagctcgaatacctttgctccactgccacacgcagcaccgggactgggcgtctggagcttaagtctgggggtctgagcctgggaccggcaaatccgcgcagcgcatcgcgcccagtctcggagactgcaaccaccgccaaggagtacgcgcggcaggaaacttctgcggcccaatttcttccccagctttggcatctccgaaggcacgtacccgccctcggcacaagctctctcgtcttccacttcgacctcgaggtggagaaagaggctggcaagggctgtgcgcgtcgctggtgtggggagggcagcaggctgcccctccccgcttctgcagcgagttttcccagccaggaaaagggagggagctgtttcaggaatttcagtgccttcacctagcgactgacacaagtcgtgtgtataggaag

SEQ ID NO: 42 - DNA, 452 bp, Homo sapiens
  source 1..452: mol_type = other DNA
ggagcctgaagtcagaaaagatggggcctcgttactcactttctagcccagcccctggccctgggtcccgcagagccgtcatcgcaggctcctgcccagcctctggggtcgggtgagcaaggtgttctcttcggaagcgggaagggctgcgggtcggggacgtcccttggctgccacccctgattctgcatccttttcgctcgaatccctgcgctaggcatcctccccgatcccccaaaagcccaagcactgggtctgggttgaggaagggaacgggtgcccaggccggacagaggctgaaaggaggcctcaaggttcctctttgctacaaagtggagaagttgctctactctggagggcagtggccttttccaaacttttccacttaggtccgtaagaaaagcaattcatacacgatcagcgctttcggtgcgaggatggaaagaaacttc

SEQ ID NO: 43 - DNA, 1992 bp, Homo sapiens
  source 1..1992: mol_type = other DNA
ttttcctgttacagagctgagcccactcatgtggtgccaagtagcgactatctctcggccacctccacccagagcaatgtgggcgcccccagcgggtgggagcgattgccgagcggcgcaagggcgtttaacgcctaaccccctcctcctgggttgccaagccgctaggtcgccgtttccaacgtggctgcgcgggactgaagtccgacgactcctcgtcctcagtaggagacacacctcccactgcccccagccacgcgagctatgggcagaatcggggcaacggtaatatctggatggggcaggctcccctgaggctgtgcttaagaaaaaaggaatctggagtagcctgaggggccccacgagggggcctcctttgcgatcgtctcccagccttaggccaaggctacggaggcaggcggccgagtgttggcgcccagcccggccgaggactggatggaggacgagaagcagcctgcctctgggcgacagctgcggacgcagcctcgccgcctcgccgcctcagcctcggtcccagcgtctctaaagccgcgcccattttacagatgcagggcagggagacaagaggcatctccgggggccgagtagaatgatggcgcgggttctcccggcgccctgatttcgaggctgcgcccggggccctacatgcaggcggggaggcctgggccgaaggcgtctgcaaggaggggcgagtctgcccggtccgggcagggagtgaggccacagtcagttctccctaggaggccgcgcagcgggtagggtatgggactgggggacgcaacggggacctggccgaatcagagccctcagcagagaacgccgaaaactctggggccggccgctcgcttcccgctagtgggaatggtttccggtcatccgttcccagtccagccccgggtagggagctctgatttgcaatgcacagcacttgcgaggttcgaatgcccccgcaatttgcagatggaaatactaagcctaggccgggcgtggtggctcaagcctatcatctcagccctttgggaggccaagccgggaggattgtttgagcccaagaattcaaaaccagcctgagcaacatagcgaccccgtctctacaaaataaaataaaataaattatccgggcgtggtggcacgcgcctgtggttccagctactccggaggctgaggtgggaggatcgcttgagtccgggaggtcgaggctacagtgagccgtgatcgcaccactgcactccagcctgggcgacagagtgagaccttgtctcaaaaaaggaaaaaaagaaaaagaaagtaagcttcaaagaagctctgataatagttctgggtcgtgcagcggtggcggccccgcgctctcgcccctaaagcaagcgctctttgtactgggtggaggagctttgagtagtgagggtggagatgcagcttcggggtggcgcagccaccctgacactaggcccggggtcgcagtgggacagaagagtctgccgctctgacttgggctctgagttccaagggcgcccggcacttctagcctcccaggcttgcgcgctggcgcctttgccatccgtgccgaagtggggagacctagccgcgaccaccacgagcgcagcggtgacacccagaggtcccaccgggcccctgggcagggtaaccttagcctgtccgcttcggcagctttgcgaagagtggcgcgcagctagggctgaggctcttgcggacctgcggtcgaagcaggcggctgagccagttcgatcgccaaggcctgggctgccgacagtggtgcgcgctctgttccgccgcggccgggccaggcgctctggaatagcgatggggggacacggcctccaactttctgcagagaccatcgggcagctccgggcctaagcagcgacctcaccgaaggttcctgggaacctttgccaaaatcccagcctctgcctcggtccagctaaaccgtgtgtaaacaagtgcaccaag

SEQ ID NO: 44 - DNA, 448 bp, Homo sapiens
  source 1..448: mol_type = other DNA
ataaaggaccgggtaatttcgcggaatgcggattttgagacaggcccagacggcggcggattccctgtgtcccccaactggggcgatctcgtgaacacacctgcgtcccaccccgatcctaggttggggggaaagggtatgggaaccctgagcccagagcgcgccccgctctttcctttgctccccggcttccctggccagccccctcccggctggtttcctcgctcactcggcgcctggcgtttcgggcgtctggagatcaccgcgtgtctggcaccccaacgtctagtctccccgcaggttgaccgcggcgcctggagccgggaataggggtggggagtccggagaaccaaacccgagcctgaagttgccattcgggtgactcccgagaaagcccgggagcattttggccaatgcgggtttttacctgaacttcagcatcttcacc

SEQ ID NO: 45 - DNA, 395 bp, Homo sapiens
  source 1..395: mol_type = other DNA
aattggaaaaccctggtattgtgcctgtttgggggaagaaaacgtcaataaaaattaattgatgagttggcagggcgggcggtgcgggttcgcggcgaggcgcagggtgtcatggcaaatgttacggctcagattaagcgattgttaattaaaaagcgacggtaattaatactcgctacgccatatgggcccgtgaaaaggcacaaaaggtttctccgcatgtggggttccccttctcttttctccttccacaaaagcaccccagcccgtgggtcccccctttggccccaaggtaggtggaactcgtcacttccggccagggaggggatggggcggtctccggcgagttccaagggcgtccctcgttgcgcactcgcccgcccaggttctttgaa

SEQ ID NO: 46 - DNA, 491 bp, Homo sapiens
  source 1..491: mol_type = other DNA
gggaagcgatcgtctcctctgtcaactcgcgcctgggcacttagcccctcccgtttcagggcgccgcctccccggatggcaaacactataaagtggcggcgaataaggttcctcctgctgctctcggtttagtccaagatcagcgatatcacgcgtcccccggagcatcgcgtgcaggagccatggcgcgggagctataccacgaagagttcgcccgggcgggcaagcaggcggggctgcaggtctggaggattgagaagctggagctggtgcccgtgccccagagcgctcacggcgacttctacgtcggggatgcctacctggtgctgcacacggccaagacgagccgaggcttcacctaccacctgcacttctggctcggtaagggacggcgggcggcgggaccccgacgcaccaaggccggcgaggggagggcgtaggggtctgagatttgcaggcgtgggagtaaaggggaccgcaaactgagctag

SEQ ID NO: 47 - DNA, 1284 bp, Homo sapiens
  source 1..1284: mol_type = other DNA
ctcaggggcgggaagtggcgggtgggagtcacccaagcgtgactgcccgaggcccctcctgccgcggcgaggaagctccataaaagccctgtcgcgacccgctctctgcaccccatccgctggctctcacccctcggagacgctcgcccgacagcatagtacttgccgcccagccacgcccgcgcgccagccaccgtgagtgctacgacccgtctgtctaggggtgggagcgaacggggcgcccgcgaacttgctagagacgcagcctcccgctctgtggagccctggggccctgggatgatcgcgctccactccccagcggactatgccggctccgcgccccgacgcggaccagccctcttggcggctaaattccacttgttcctctgctcccctctgattgtccacggcccttctcccgggcccttcccgctgggcggttcttctgagttaccttttagcagatatggagggagaacccgggaccgctatcccaaggcagctggcggtctccctgcgggtcgccgccttgaggcccaggaagcggtgcgcggtaggaaggtttccccggcagcgccatcgagtgaggaatccctggagctctagagccccgcgccctgccacctccctggattcttgggctccaaatctctttggagcaattctggcccagggagcaattctctttccccttccccaccgcagtcgtcaccccgaggtgatctctgctgtcagcgttgatcccctgaagctaggcagaccagaagtaacagagaagaaacttttcttcccagacaagagtttgggcaagaagggagaaaagtgacccagcaggaagaacttccaattcggttttgaatgctaaactggcggggcccccaccttgcactctcgccgcgcgcttcttggtccctgagacttcgaacgaagttgcgcgaagttttcaggtggagcagaggggcaggtcccgaccggacggcgcccggagcccgcaaggtggtgctagccactcctgggttctctctgcgggactgggacgagagcggattgggggtcgcgtgtggtagcaggaggaggagcgcggggggcagaggagggaggtgctgcgcgtgggtgctctgaatccccaagcccgtccgttgagccttctgtgcctgcagatgctaggtaacaagcgactggggctgtccggactgaccctcgccctgtccctgctcgtgtgcctgggtgcgctggccgaggcgtacccctccaagccggacaacccgggcgaggacgcaccag

SEQ ID NO: 48 - DNA, 554 bp, Homo sapiens
  source 1..554: mol_type = other DNA
tggagaaccttgggctctgtggcctcaaaggtaggggtgatttcgaggggccggcacctcacagggcaggttccaccgcggaaacgcagtcatcgcccagcgaccctgctcctggccctcagcctccccccaggtttctttttctcttgaatcaagccgaggtgcgccaatggccttccttgggtcggatccggggggccagggccagcttacctgctttcaccgagcagtggatatgtgccttggactcgtagtacacccagtcgaagccggcctccaccgccaggcgggccagcatgccgtacttgctgcggtcgcggtcagacgtggtgatgtccactgcgcggccctcgtagtgcagagactcctctgagtggtggccatcttcgtcccagccctcggtcacccgcagtttcactcctggccactggttcatcaccgagatggccaaagcgttcaacttgtccttacacctctgcgaagacaaggggacccccaccgacggacacgttagcctgggcaaccgccacccctcccggcccctccatcagcct

SEQ ID NO: 49 - DNA, 772 bp, Homo sapiens
  source 1..772: mol_type = other DNA
tctcacgacccatccgttaacccaccgttcccaggagctccgaggcgcagcggcgacagaggttcgccccggcctgctagcattggcattgcggttgactgagcttcgcctaacaggcttggggagggtgggctgggctgggctgggctgggctgggtgctgcccggctgtccgcctttcgttttcctgggaccgaggagtcttccgctccgtatctgcctagagtctgaatccgactttctttcctttgggcacgcgctcgccagtggagcacttcttgttctggccccgggctgatctgcacgcggacttgagcaggtgccaaggtgccacgcagtcccctcacggctttcggggggtcttggagtcgggtggggagggagacttaggtgtggtaacctgcgcaggtgccaaagggcagaaggagcagccttggattatagtcacggtctctccctctcttccctgccatttttagggctttctctacgtgctgttgtctcactgggtttttgtcggagccccacgccctccggcctctgattcctggaagaaagggttggtcccctcagcacccccagcatcccggaaaatggggagcaaggctctgccagcgcccatcccgctccacccgtcgctgcagctcaccaattactccttcctgcaggccgtgaacaccttcccggccacggtggaccacctgcagggcctgtacggtctcagcgcggtacagaccatgcacatgaaccactggacgctggggtatcccaat

SEQ ID NO: 50 - DNA, 1362 bp, Homo sapiens
  source 1..1362: mol_type = other DNA
tggtttcctttcgcttctcgcctcccaaacacctccagcaagtcggagggcgcgaacgcggagccagaaacccttccccaaagtttctcccgccaggtacctaattgaatcatccataggatgacaaatcagccagggccaagatttccagacacttgagtgacttcccggtccccgaggtgacttgtcagctccagtgagtaacttggaactgtcgctcggggcaaggtgtgtgtctaggagagagccggcggctcactcacgctttccagagagcgacccgggccgacttcaaaatacacacagggtcatttatagggactggagccgcgcgcaggacaacgtctccgagactgagacattttccaaacagtgctgacattttgtcgggccccataaaaaatgtaaacgcgaggtgacgaacccggcggggagggttcgtgtctggctgtgtctgcgtcctggcggcgtgggaggttatagttccagacctggcggctgcggatcgccgggccggtacccgcgaggagtgtaggtaccctcagcccgaccacctcccgcaatcatggggacaccggcttggatgagacacaggcgtggaaaacagccttcgtgaaactccacaaacacgtggaacttgaaaagacaactacagccccgcgtgtgcgcgagagacctcacgtcaccccatcagttcccacttcgccaaagtttcccttcagtggggactccagagtggtgcgccccatgcccgtgcgtcctgtaacgtgccctgattgtgtacccctctgcccgctctacttgaaatgaaaacacaaaaactgttccgaattagcgcaactttaaagccccgttatctgtcttctacactgggcgctcttaggccactgacagaaacatggtttgaaccctaattgttgctatcagtctcagtcagcgcaggtctctcagtgacctgtgacgccgggagttgaggtgcgcgtatccttaaacccgcgcgaacgccaccggctcagcgtagaaaactatttgtaatccctagtttgcgtctctgagctttaactcccccacactctcaagcgcccggtttctcctcgtctctcgcctgcgagcaaagttcctatggcatccacttaccaggtaaccgggatttccacaacaaagcccggcgtgcgggtcccttcccccggccggccagcgcgagtgacagcgggcggccggcgctggcgaggagtaacttggggctccagcccttcagagcgctccgcgggctgtgcctccttcggaaatgaaaacccccatccaaacggggggacggagcgcggaaacccggcccaagtgccgtgtgtgcgcgcgcgtctg

SEQ ID NO: 51 - DNA, 476 bp, Homo sapiens
  source 1..476: mol_type = other DNA
gaaagccatccttaccattcccctcaccctccgccctctgatcgcccacccgccgaaagggtttctaaaaatagcccagggcttcaaggccgcgcttctgtgaagtgtggagcgagcgggcacgtagcggtctctgccaggtggctggagccctggaagcgagaaggcgcttcctccctgcatttccacctcaccccacccccggctcatttttctaagaaaaagtttttgcggttccctttgcctcctacccccgctgccgcgcggggtctgggtgcagacccctgccaggttccgcagtgtgcagcggcggctgctgcgctctcccagcctcggcgagggttaaaggcgtccggagcaggcagagcgccgcgcgccagtctatttttacttgcttcccccgccgctccgcgctcccccttctcagcagttgcacatgccagctctgctgaaggcatcaatgaaaacagcagtag

SEQ ID NO: 52 - DNA, 300 bp, Homo sapiens
  source 1..300: mol_type = other DNA
atcgaaaatgtcgacatcttgctaatggtctgcaaacttccgccaattatgactgacctcccagactcggccccaggaggctcgtattaggcagggaggccgccgtaattctgggatcaaaagcgggaaggtgcgaactcctctttgtctctgcgtgcccggcgcgcccccctcccggtgggtgataaacccactctggcgccggccatgcgctgggtgattaatttgcgaacaaacaaaagcggcctggtggccactgcattcgggttaaacattggccagcgtgttccgaaggcttgt

SEQ ID NO: 53 - DNA, 1246 bp, Homo sapiens
  source 1..1246: mol_type = other DNA
atcaacatcgtggctttggtcttttccatcatggtgagtgaatcacggccagaggcagcctgggaggagagacccgggcggctttgagcccctgcaggggagtccgcgcgctctctgcggctcccttcctcacggcccggcccgcgctaggtgttctttgtcctcgcacctcctcctcacctttctcgggctctcagagctctccccgcaatcatcagcacctcctctgcactcctcgtggtactcagagccctgatcaagcttcccccaggctagctttcctcttctttccagctcccagggtgcgtttcctctccaacccggggaagttcttccgtggactttgctgactcctctgaccttcctaggcacttgcccggggcttctcaaccctcttttctagagccccagtgcgcgccaccctagcgagcgcagtaagctcataccccgagcatgcaggctctacgttcctttccctgccgctccgggggctcctgctctccagcgcccaggactgtctctatctcagcctgtgctcccttctctctttgctgcgcccaagggcaccgcttccgccactctccggggggtccccaggcgattcctgatgccccctccttgatcccgtttccgcgctttggcacggcacgctctgtccaggcaacagtttcctctcgcttcttcctacacccaacttcctctccttgcctccctccggcgccccctttttaacgcgcccgaggctggctcacacccactacctctttaggcctttcttaggctccccgtgtgcccccctcaccagcaaagtgggtgcgcctctcttactctttctacccagcgcgtcgtagttcctccccgtttgctgcgcactggccctaacctctcttctcttggtgtcccccagagctcccaggcgcccctccaccgctctgtcctgcgcccggggctctcccgggaatgaactaggggattccacgcaacgtgcggctccgcccgccctctgcgctcagacctcccgagctgcccgcctctctaggagtggccgctggggcctctagtccgcccttccggagctcagctccctagccctcttcaaccctggtaggaacacccgagcgaaccccaccaggagggcgacgagcgcctgctaggccctcgccttattgactgcagcagctggcccgggggtggcggcggggtgaggttcgtaccggcactgtcccgggacaacccttgcagttgc

SEQ ID NO: 54 - DNA, 984 bp, Homo sapiens
  source 1..984: mol_type = other DNA
acaaataaaacaccctctagcttcccctagactttgtttaactggccgggtctccagaaggaacgctggggatgggatgggtggagagagggagcggctcaaggactttagtgaggagcaggcgagaaggagcacgttcaggcgtcaagaccgatttctccccctgcttcgggagacttttgaacgctcggagaggcccggcatctcaccactttacttggccgtaggggcctccggcacggcaggaatgagggagggggtccgattggacagtgacggtttggggccgttcggctatgttcagggaccatatggtttggggacagccccagtagttagtaggggacgggtgcgttcgcccagtccccggatgcgtagggaggcccagtggcaggcagctgtcccaagcagcgggtgcgcgtccctgcgcgctgtgtgttcattttgcagagccagccttcggggaggtgaaccagctgggaggagtgttcgtgaacgggaggccgctgcccaacgccatccggcttcgcatcgtggaactggcccaactgggcatccgaccgtgtgacatcagccgccagctacgggtctcgcacggctgcgtcagcaagatcctggcgcgatacaacgagacgggctcgatcttgccaggagccatcgggggcagcaagccccgggtcactacccccaccgtggtgaaacacatccggacctacaagcagagagaccccggcatcttcgcctgggagatccgggaccgcctgctggcggacggcgtgtgcgacaagtacaatgtgccctccgtgagctccatcagccgcattctgcgcaacaagatcggcaacttggcccagcagggtcattacgactcatacaagcagcaccagccgacgccgcagccagcgctgccctacaaccacatctactcgtaccccagccctatcacggcggcggccgccaaggtgcccacgccacccggggtgc

SEQ ID NO: 55 - DNA, 545 bp, Homo sapiens
  source 1..545: mol_type = other DNA
aggaggcgcaacgcgctgccagggcggctttatcctgccgccacagggcggggaccagcccggcagccgggtgtccagcgccgctcacgtgcctcgcctggagcttagctctcagactccgaagagggcgactgagacttgggcctgggagttggcttcggggtacccaaggcgacgacagctgagttgtaccacgaagctcaggccgaggcctcctcccttgtctggccttcgaatccatactggcagcctctcctctcaggcactccgcgggccgggccactaggccccctgctcctggagctgcgctatgatccgggtcttgagatgcgcgcgattctctctgaaccggtggagaggaggctctgccccgcgcggagcgaggacagcggcgcccgagcttcccgcgcctctccagggcccaatggcaagaacagcctccgaagtgcgcggatgacaggaaaagatcttcagttcttctgccgctagagaagtgcgggatacaagcctctattggatccacaacctggagtcctgccttcgga

SEQ ID NO: 56 - DNA, 1533 bp, Homo sapiens
  source 1..1533: mol_type = other DNA
atctgcgtgcccttttctgggcgagccctgggagatccagggagaactgggcgctccagatggtgtatgtctgtaccttcacagcaaggcttcccttggatttgaggcttcctattttgtctgggatcggggtttctccttgtcccagtggcagccccgcgttgcgggttccgggcgctgcgcggagcccaaggctgcatggcagtgtgcagcgcccgccagtcgggctggtgggttgtgcactccgtcggcagctgcagaaaggtgggagtgcaggtcttgcctttcctcaccgggcggttggcttccagcaccgaggctgacctatcgtggcaagtttgcggcccccgcagatccccagtggagaaagagggctcttccgatgcgatcgagtgtgcgcctccccgcaaagcaatgcagaccctaaatcactcaaggcctggagctccagtctcaaaggtggcagaaaaggccagacctaactcgagcacctactgccttctgcttgccccgcagagccttcagggactgactgggacgcccctggtggcgggcagtcccatccgccatgagaacgccgtgcagggcagcgcagtggaggtgcagacgtaccagccgccgtggaaggcgctcagcgagtttgccctccagagcgacctggaccaacccgccttccaacagctggtgaggccctgccctacccgccccgacctcgggactctgcgggttggggatttagccacttagcctggcagagaggggagggggtggccttgggctgaggggctgggtacagccctaggcggtgggggagggggaacagtggcgggctctgaaacctcacctcggcccattacgcgccctaaaccaggtctccctggattaaagtgctcacaagagaggtcgcaggattaaccaacccgctcccccgccctaatccccccctcgtgcgcctggggacctggcctccttctccgcagggcttgctctcagctggcggccggtccccaagggacactttccgactcggagcacgcggccctggagcaccagctcgcgtgcctcttcacctgcctcttcccggtgtttccgccgccccaggtctccttctccgagtccggctccctaggcaactcctccggcagcgacgtgacctccctgtcctcgcagctcccggacacccccaacagtatggtgccgagtcccgtggagacgtgagggggacccctccctgccagcccgcggacctcgcatgctccctgcatgagactcacccatgctcaggccattccagttccgaaagctctctcgccttcgtaattattctattgttatttatgagagagtaccgagagacacggtctggacagcccaaggcgccaggatgcaacctgctttcaccagactgcagacccctgctccgaggactcttagtttttcaaaaccagaatctgggacttaccagggttagctctgccctctcctctcctctctacgtggccgccgctctgtctctccacgccccacctgtgt

SEQ ID NO: 57 - DNA, 702 bp, Homo sapiens
  source 1..702: mol_type = other DNA
aggtctcttcagactgcccattctccgggcctcgctgaatgcgggggctctatccacagcgcgcggggccgagctcaggcaggctggggcgaagatctgattctttccttcccgccgccaaaccgaattaatcagtttcttcaacctgagttactaagaaagaaaggtccttccaaataaaactgaaaatcactgcgaatgacaatactatactacaagttcgttttggggccggtgggtgggatggaggagaaagggcacggataatcccggagggccgcggagtgaggaggactatggtcgcggtggaatctctgttccgctggcacatccgcgcaggtgcggctctgagtgctggctcggggttacagacctcggcatccggctgcaggggcagacagagacctcctctgctagggcgtgcggtaggcatcgtatggagcccagagactgccgagagcactgcgcactcaccaagtgttaggggtgcccgtgatagaccgccagggaaggggctggttcggagggaattcccgctaccgggaaggtcggaactcggggtgatcaaacaaggaatgcatctcacctccgtgggtgcttgtgctgcgcaaggaattattaccggagcggttgcgatggcctttgcccggcgacccaagaagagtaagcaaactaccgtccacccagcggatcaggtccaat

SEQ ID NO: 58 - DNA, 3180 bp, Homo sapiens
  source 1..3180: mol_type = other DNA
gatgtcctgtttctagcagcctccagagccaagctaggcgagaggcgtaggaggcagagagagcgggcgcgggaggccagggtccgcctgggggcctgaggggacttcgtggggtcccgggagtggcctagaaacagggagctgggagggccgggaagagcttgaggctgagcgggggacgaacgggcagcgcaaaggggagatgaacggaatggccgaggagccacgcattcgccttgtgtccgcggacccttgttcccgacaggcgaccaagccaaggccctccggactgacgcggcctgagcagcagcgagtgtgaagtttggcacctccggcggcgagacggcgcgttctggcgcgcggctcctgcgtccggctggtggagctgctgcgccctatgcggcctgccgagggcgccgccgagggcccgcgagctccgtggggtcggggtggggggacccgggagcggacagcgcggcccgaggggcaggggcaggggcgcgcctggcctggggtgtgtctgggccccggctccgggctcttgaaggaccgcgagcaggaggcttgcgcaatcccttggctgagcgtccacggagaaagaaaaagagcaaaagcagagcgagagtggagcgagggatgggggcgggcaaagagccatccgggtctccaccaccgccctgacacgcgacccggctgtctgttggggaccgcacgggggctcgggcgagcaggggagggaggagcctgcgcggggctcgtgttcgcccaggaatcccggagaagctcgaagacggtctggtgttgaacgcacacgtggactccatttcattaccaccttgcagctcttgcgccacggaggctgctgctgcccggcggctgctacccaccgagacccacgtggcccctccccaggggtgtaggggtgacggttgtcttctggtgacagcagaggtgttgggtttgcgactgatctctaacgagcttgaggcgcaaacctaggattccctgagtgttggggtgcggcgggggggcaagcaaggtgggacgacgcctgcctggtttccctgactagttgcggggggtgggggccggctctcaggggccaccagaagctgggtgggtgtacaggaaaatatttttctcctgccgtgtttggctttttcctggcatttttgcccagggcgaagaactgtcgcgcggggcagctccaccgcggagggagaggggtcgcgaggctggcgcgggaagcgctgtaggtggcagtcatccgtccacgccgcacaggccgtctgcgccgtcggaccatcgggaggtctgcagcaactttgtcccggccagtccccttgtccgggaaggggctgagcttcccgacactctaccctccccctcttgaaaatcccctggaaaatctgtttgcaatgggtgtttccgcggcgtccaggtctgggctgccgggggaggccgagcggctgctgcagcctccctgctgccaggggcgtcggactccgcttcgctcactacgcccaggcccctcaggggcccacgctcaggacttcggggccacacagcaggacccggtgccccgacgacgagtttgcgcaggacccgggctgggccagccgcggagctggggaggaaggggcgggggtcggtgcagcggatcttttctgttgctgcctgtgcggcggcaggaagcgtcttgaggctccccaagactacctgaggggccgcccaagcacttcagaagcccaaggagcccccggccacccccgctcctggcctttttgccaacgactttgaaagtgaaatgcacaagcaccagcaattgacttcccttccgtggttatttattttgtctttgtggatggtgggcagatggggagagaggcccctacctaacctcggtggctggtccctagaccacccctgccagccggtgtggggaggagctcaggtccgcgggagagcgaatgggcgccaggaggtgggacagaatcctgggaaggtacagcggacgccctggaagctcccctgatgccccagagggcccttcctgggaaacctcccgggggggtgccccataccatcccacccggctgtcttggcccctcccagggagccgcaggagaaactagccctacacctgggattcccagagccttctgctggggctcctgcccccgacttcggataaccagctccgcacaggtccccgagaagggccgctggcctgcttatttgatactgccccctcccagacaggggctggtcgagcccctggttctgctgccagactgaagccttccagacgccacctcggtttgggcccccagggccctcaggggccccaggagaggagagctgctatctagctcagccacaggctcgctcctggtgggggccaggctgaaggagtggaccctggagaggtcgggaaccttttaacagccgtgggctggagggtggctactaagtgttcggtctgggaagaggcatgacccgcaccatcccggggaaataaacgacttcttaagggaatcttctcgctgagcgggtgctctgggccaggagattgccaccgccagcccacggaacccagatttgggctctgccttgagcgggccgcctgtggcttcccgggtcgctcccccgactcagaaagctctcaagttggtatcgttttcccggccctcggaggtggattgcagatcaccgagaggggatttaccagtaaccactacagaatctacccgggctttaacaagcgctcatttctctcccttgtccttagaaaaacttcgcgctggcgttgatcatatcgtacttgtagcggcagcttaggggcagcggaactggtggggttgtgcgtgcagggggaggctgtgagggagccctgcactccgcccctccacccttctggaggagtggctttgtttctaagggtgcccccccaacccccgggtccccacttcaatgtttctgctctttgtcccaccgcccgtgaaagctcggctttcatttggtcggcgaagcctccgacgcccccgagtcccaccctagcgggccgcgcggcactgcagccgggggttcctgcggactggcccgacagggtgcgcggacggggacgcgggccccgagcaccgcgacgccagggtcctttggcagggcccaagcacccct

SEQ ID NO: 59 - DNA, 1038 bp, Homo sapiens
  source 1..1038: mol_type = other DNA
tggcggccggcgggcacagccggctcattgttctgcactacaaccactcgggccggctggccgggcgcggggggccggaggatggcggcctgggggccctgcgggggctgtcggtggccgccagctgcctggtggtgctggagaacttgctggtgctggcggccatcaccagccacatgcggtcgcgacgctgggtctactattgcctggtgaacatcacgctgagtgacctgctcacgggcgcggcctacctggccaacgtgctgctgtcgggggcccgcaccttccgtctggcgcccgcccagtggttcctacgggagggcctgctcttcaccgccctggccgcctccaccttcagcctgctcttcactgcaggggagcgctttgccaccatggtgcggccggtggccgagagcggggccaccaagaccagccgcgtctacggcttcatcggcctctgctggctgctggccgcgctgctggggatgctgcctttgctgggctggaactgcctgtgcgcctttgaccgctgctccagccttctgcccctctactccaagcgctacatcctcttctgcctggtgatcttcgccggcgtcctggccaccatcatgggcctctatggggccatcttccgcctggtgcaggccagcgggcagaaggccccacgcccagcggcccgccgcaaggcccgccgcctgctgaagacggtgctgatgatcctgctggccttcctggtgtgctggggcccactcttcgggctgctgctggccgacgtctttggctccaacctctgggcccaggagtacctgcggggcatggactggatcctggccctggccgtcctcaactcggcggtcaaccccatcatctactccttccgcagcagggaggtgtgcagagccgtgctcagcttcctctgctgcgggtgtctccggctgggcatgcgagggcccggggactgcctggcccgggccgtcgaggctcactccggagcttccaccaccgacagctctctgaggccaagggacagctttc

SEQ ID NO: 60 - DNA, 374 bp, Homo sapiens
  source 1..374: mol_type = other DNA
tagtaaggcaccgaggggtggctcctctccctgcagcggctgtcgcttaccatcctgtagaccgtgacctcctcacacagcgccaggacgaggatcgcggtgagccagcaggtgactgcgatcctggagctggtcgcagcaggccatcctgcacgcggtggaggcgccccctgcaggccgcagcgcatccccagcttctggacgcactgtgagcggttatgcagcagcacgctcatatgagatgccccgcagggtgctatgcaggcccacgtccccacaaagcccatggcaggcgcccgggtgccggagcacgcacttggccccatggatctctgtgcccagggctcagccaggcatctggccgctaaaggttt

SEQ ID NO: 61 - DNA, 246 bp, Homo sapiens
  source 1..246: mol_type = other DNA
tctcatctgagcgctgtctttcaccagagctctgtaggactgaggcagtagcgctggcccgcctgcgagagcccgaccgtggacgatgcgtcgcgcccttcccatcgcggcctgggcgggcccgcctgccctcggctgagcccggtttccctaccccggggcacctcccctcgcccgcacccggccccagtccctcccaggcttgcgggtagagcctgtctttgcccagaaggccgtctccaagct

SEQ ID NO: 62 - DNA, 222 bp, Homo sapiens
  source 1..222: mol_type = other DNA
cagtccccgaggccctccccggtgactctaaccagggatttcagcgcgcggcgcggggctgcccccaggcgtgacctcacccgtgctctctccctgcagaatctcctacgacccggcgaggtaccccaggtacctgcctgaagcctactgcctgtgccggggctgcctgaccgggctgttcggcgaggaggacgtgcgcttccgcagcgcccctgtctacat

SEQ ID NO: 63 - DNA, 2209 bp, Homo sapiens
  source 1..2209: mol_type = other DNA
agagagacattttccacggaggccgagttgtggcgcttggggttgtgggcgaaggacggggacacgggggtgaccgtcgtggtggaggagaaggtctcggaactgtggcggcggcggcccccctgcgggtctgcgcggatgaccttggcgccgcggtgggggtccgggggctggctggcctgcaggaaggcctcgactcccgacacctgctccatgaggctcagcctcttcacgcccgacgtcgggctggccacgcgggcagcttctggcttcgggggggccgcgataggttgcggcggggtggcggccacaccaaaagccatctcggtgtagtcaccattgtccccggtgtccgaggacaacgatgaggcggcgcccgggccctgggcggtggcaacggccgaggcggggggcaggcggtacagctcccccggggccggcggcggtggcggcggctgcagagacgacgacggggacgcggacggacgcgggggcaacggcggatacggggaggaggcctcgggggacaggaggccgtccaaggagcccacggggtggccgctcggggcgcccggcttaggagacttgggggagctgaagtcgaggttcatgtagtcggagagcggagaccgctgccggctgtcgctgctggtgcccggggtgcctgagcccagcgacgaggccgggctgctggcggacaagagcgaggaggacgaggccgccgacgccagcaggggaggcgcgggcggcgacaggcgggccccgggctcgccaaagtcgatgttgatgtactcgccggggctcttgggctccggtggcagtgggtactcgtgcatgctgggcaggctgggcagcccctccagggacaggcgcgtgggcctcaccgcccggccgcgctggcccaagaagccctccgggcggccgccgctaggccgcacgggcgaaggcactacagggtgagggggctgcgtggggccggccccgaaggcgctggccgcctggctgggccctggcgtggcctgaggctccagacgctcctcctccaggatgcgccccacgggggagctcatgagcacgtactggtcgctgtccccgccacaggtgtagggggccttgtaggagcggggcaaggagctgtagcagcagccgggaacgcccctgagcggctccccgccggggtgcagggctgcggagaagaagtcgggcggggtgcccgtggtgaccgcgtcgctgggggacacgttgaggtagtccccgttgggcagcagcttgccatctgcatgctccatggacagcttggaaccgcaccacatgcgcatgtacccactgtcctcgggggagctctcggcgggcgagctggccttgtagccgcccccgctcgccgggaatgtcctgcccgccgcagaggtgggtgctggccccgcaggccccgcagaaggcacggcggcggcggcggcggcggcggccctgggctgcaagatctgcttgggggcggacacgctggcggggctcatgggcatgtagtcgtcgctcctgcagctgccgctcccactgcccgcgagggccgcgccgggcgtcatgggcatgtagccgtcgtctgcccccaggttgctgctggagctcctgtgggagccgatctcgatgtctccgtagtcctctgggtaggggtggtaggccaccttgggagaggacgcggggcaggacgggcagaggcggcccgcgctgcccgagaaggtggcccgcatcagggtgtattcatccagcgaggcagaggagggctggggcaccggccgctgccgggctggcgtggtcagggagtaggtcctcttgcgcagccctcggtccaggtcctgggccgcgtcccccgagacccggcggtaggagcggccacagtggctcaggggcctgtccatggtcatgtacccgtagaactcaccgccgccgccgccgtctcgggccgggggcgtctccgcgatggactcgggcgtgttgcttcggtggctgcagaaggcgcgcaggtcgcctgggctggagccgtactcgtccagggacatgaagccggggtcgctgggggagcccgaggcggaggcgctgccgctggagggccgctggccggggccgtggtgcagcggatgcggcagaggcgggtgcgggccgggcggcggcgggtaggagcccgagccgtggccgctgctggacgacagggagc

SEQ ID NO: 64 - DNA, 149 bp, Homo sapiens
  source 1..149: mol_type = other DNA
taacctaaagaatgaagtcatgccccggcctgcacccgggaaactgcacacagcgaaagatcgccactgagataaagagctgaaagctattccccaattcagctgtttcagccgtgcggtctcacaatgggctcacagacggcagcatc

SEQ ID NO: 65 - DNA, 832 bp, Homo sapiens
  source 1..832: mol_type = other DNA
gtttccacaatccacctcgtagctggggcgtgccgcttgcctcggcttgtcccggcagaacactcttacctttaatggcgactgaaaagttgccacgagttcctgatcattgtggtaggtgctgcgtgaagctgagacgtgcgtgagccacatcccagggggctttgagcccccaccgcggcggcggctgaggggaggcttgtcgtactcgcacaggaggacacagggctgcagtgttcactccagggcctcttatcattgggatctgaggaattttccgagaggaagtgcgaattaacaatgatgaaaggtttgtgagtgagtgacaggcacgttctattgagcactgcatggggcattatgtgccaccagagacgggggcagaggtcaagagccctcgagggctgggagagttcggaggatagaagtcatcagagcacaatgaagccagaccctgcagccgccttccccttcgggggcttccttagaatgcagcattgcggggactgagctgtcccaggtgaaggggggccgtcacggtgtgtggacgcccctcggctcagccctctaagagactcggcagccaggatgggctcaaggcatgagccctcaaaggaggttaggaaggagcgagggagaaaagatatgcttgtgtgacgtcctggccgaagtgagaacaattgtatcagataatgagtcatgtcccattgaggggtgccgacaaggactcgggaggaggccacggagccctgtactgaggagacgcccacagggagcctcgggggcccagcgtcccgggatcactggatggtaaagccgccctgcctggcgt

SEQ ID NO: 66 - DNA, 256 bp, Homo sapiens
  source 1..256: mol_type = other DNA
tccagctgcagcgagggcggccaggcccccttctccgacctgcaggggtagcgcggcctcggcgccggagacccgcgcgctgtctggggctgcggtggcgtggggagggcgcggcccccggacgccccgaggaaggggcacctcaccgcccccacccagagcgcctggccgtgcgggctgcagaggacccctccggggcagaggcaggttccacggaagaccccggcccgctggggcttccccggagactccagag

SEQ ID NO: 67 - DNA, 184 bp, Homo sapiens
  source 1..184: mol_type = other DNA
acttactgcttccaaaagcgctgggcacagccttatatgactgaccccgcccccgagtcccaggccgccccatgcaaccgcccaaccgcccaaccgccactccaaaggtcaccaaccactgctccaggccacgggctgcctctccccacggctctagggcccttcccctccaccgcaggctgac

SEQ ID NO: 68 - DNA, 118 bp, Homo sapiens
  source 1..118: mol_type = other DNA
tgccacacccaggtaccgcccgcccgcgcgagagccgggcaggtgggccgcggatgctcccagaggccggcccagcagagcgatggacttggacaggctaagatggaagtgacctgag

SEQ ID NO: 69 - DNA, 1534 bp, Homo sapiens
  source 1..1534: mol_type = other DNA
tcgccagcgcagcgctggtccatgcaggtgccacccgaggtgagcgcggaggcaggcgacgcggcagtgctgccctgcaccttcacgcacccgcaccgccactacgacgggccgctgacggccatctggcgcgcgggcgagccctatgcgggcccgcaggtgttccgctgcgctgcggcgcggggcagcgagctctgccagacggcgctgagcctgcacggccgcttccggctgctgggcaacccgcgccgcaacgacctctcgctgcgcgtcgagcgcctcgccctggctgacgaccgccgctacttctgccgcgtcgagttcgccggcgacgtccatgaccgctacgagagccgccacggcgtccggctgcacgtgacaggcgaggcggcgtgggagcgggtccccggcctcccttcccgccctcccgcctgccccgccccaagggctacgtgggtgccaggcgctgtgctgagccaggaagggcaacgagacccagccctctcctctaccccagggatctcacacctgggggtagtttaggaccacctgggagcttgacacaaatgcagaatccaggtcccaggaagggctgaggtgggcccgggaataggcattgccgtgactctcgtagagtgactgtccccagtggctctcagacgaagaggcgagaaagacaagtgaatggcaatcctaaatatgccaagaggtgcaatgtggtgtgtgctaccagcccggaaagacactcgcagcccctctacccaggggtgcacagacagcccaccaagtagtgcctagcactttgccagaccctgatatacaaagatgcctgaaccagggtcccgtccctagagcagtggctctccactctagcccccaccctgctctgcgacaataatggccacttagcatttgctagggagccgggacctagtccaagcacccacaagcatgaatttgccaaatcttttcagcaacctcttaaggcaactgctatcatgatcctcactttacacatggagaagcagaagcagagatgatagaatctttcgcccaaggccacatctgtattgggacgggggcagcctggcacccaagtgcccattcctcccttctgaccagcccccacccctccggctctggcgtccaaagggctaaggggaggggtgcccttgtgacagtcacccgccttctcccctgcagccgcgccgcggatcgtcaacatctcggtgctgcccagtccggctcacgccttccgcgcgctctgcactgccgaaggggagccgccgcccgccctcgcctggtccggcccggccctgggcaacagcttggcagccgtgcggagcccgcgtgagggtcacggccacctagtgaccgccgaactgcccgcactgacccatgacggccgctacacgtgtacggccgccaacagcctgggccgctccgaggccagcgtctacctgttccgcttccatggcgccagcggggcctcgacggtcgccctcctgctcggcgctctcggcttcaaggcgct

SEQ ID NO: 70 - DNA, 269 bp, Homo sapiens
  source 1..269: mol_type = other DNA
atgaacttcaagggcgacatcatcgtggtctacgtcagccagacctcgcaggagggcgcggcggcggctgcggagcccatgggccgcccggtgcaggaggagaccctggcgcgccgagactccttcgcggggaacggcccgcgcttcccggacccgtgcggcggccccgaggggctgcgggagccggagaaggcctcgaggccggtgcaggagcaaggcggggccaaggcttgagcgccccccatggctgggagcccgaagctcggagc

SEQ ID NO: 71 - DNA, 282 bp, Homo sapiens
  source 1..282: mol_type = other DNA
tcagtgttatgtggggagcgctagatcgtgcacacagtaggcgtcaggaagtgttttccccagtaatttattctccatggtactttgctaaagtcatgaaataactcagattttgttttccaaggaaggagaaaggcccagaatttaagagcaggcagacacacaaccgggcacccccagaccctggcccttccagcagtcaggaattgacttgccttccaaagccccagcccggagcttgaggaacggactttcctgcgcagggggatcggggcgcactcg

SEQ ID NO: 72 - DNA, 142 bp, Homo sapiens
  source 1..142: mol_type = other DNA
gtggaaacacaacctgccttccattgtctgcgcctccaaaacacaccccccgcgcatccgtgaagctgtgtgtttctgtgttactacaggggccggctgtggaaatcccacgctccagaccgcgtgccgggcaggcccagcc

SEQ ID NO: 73 - DNA, 741 bp, Homo sapiens
  source 1..741: mol_type = other DNA
tccacacctcgggcagtcactaggaaaagggtcgccaactgaaaggcctgcaggaaccaggatgatacctgcgtcagtcccgcggctgctgcgagtgcgcgctctcctgccagggggacctcagaccctcctttacagcacaccgagggccctgcagacacgcgagcgggccttcagtttgcaaaccctgaaagcgggcgcggtccaccaggacgatctggcagggctctgggtgaggaggccgcgtctttatttggggtcctcgggcagccacgttgcagctctgggggaagactgcttaaggaacccgctctgaactgcgcgctggtgtcctctccggccctcgcttccccgaccccgcacaggctaacgggagacgcgcaggcccaccccaccggctggagaccccggcacggcccgcatccgccaggattgaagcagctggcttggacgcgcgcagttttcctttggcgacattgcagcgtcggtgcggccacaatccgtccactggttgtgggaacggttggaggtcccccaagaaggagacacgcagagctctccagaaccgcctacatgcgcatggggcccaaacagcctcccaaggagcacccaggtccatgcacccgagcccaaaatcacagacccgctacgggcttttgcacatcagctccaaacacctgagtccacgtgcacaggctctcgcacaggggactcacgcacctgagttcgcgctcacagatc

SEQ ID NO: 74 - DNA, 4498 bp, Homo sapiens
  source 1..4498: mol_type = other DNA
ctgccctcgcggatctcccccggcctcgccggcctccgcctgtcctcccaccaccctctccgggccagtaccttgaaagcgatgggcagggtcttgttgcagcgccagtgcgtaggcagcacggagcagaggaagttggggctgtcggtgcgcaccagctcgcccgggtggtcggccagcacctccaccatgctgcggtcgccgctcctcagcttgccggccagggcagcgccggcgtccggggcgcccagcggcaacgcctcgctcatcttgcctgggctcagcgcggtggaaggcggcgtgaagcggcggctcgtgctggcatctacggggatacgcatcacaacaagccgattgagttaggaccctgcaaacagctcctaccagacggcgacaggggcgcggatcttcagcaagcagctcccgggagaccaacatacacgttcaggggcctttattactgcggggggtgggggggggcgggggtggttaggggaggagggagactaagttactaacagtccaggaggggaaaacgttctggttctgcggatcggcctctgacccaggatgggctcctagcaaccgattgcttagtgcattaaaaagtggagactatcttccacgaatcttgcttgcagaggttaagttctgtctttggctgttagaaaagttcctgaaggcaaaattctcatacacttcctaaaatatttatgcgaagagtaaaacgatcagcaaacacattatttggaagttccagtagttaatgcctgtcagttttttgcaggtgagttttgtctaaagtcccaacagaacacaattatctcccgtaacaaggccacttttatcatgcaaaactggcttcagtcccgaaaagcaagagctgagacttccaaaggtagtgctactaatgtatgtgcacgtatatataaatatatacatatgctctacttcataaaatatttacaatacaatctgtggagaatttaaacacaacagaaatccattaatgtacgctgcagatttttttaagtagccttgaaaatcagcttcagtagttggagcagtgctgagctagaagtacttgtcatgttctctgttctctcaatgaattctgtcaaaacgctcagtgcagaaaattcagcgtttcagagatcttcagctaatcttaaaacaacaatcataagaaggcccagtcgatgacactcagggttctacagctctcccacatctgtgaactcgggtttggggatgttggttaagtttgtggctggtcctctggtttgttgggagttgagcagccgcagagtcacacacatgcaaacacgcactcttcggaaggcagccactgtctacatcagctgggtgactcagccctgactcgggcagcagcgagacgatactcctccaccgtcgcccagcacccgccggttagctgctccgaggcacgaacacccacgagcgccgcgtaaccgcagcaggtggagcgggccttgagggagggctccgcggcgcagatcgaaacagatcgggcggctcgggttacacacgcacgcacatcctgccacgcacactgccacgcacacgcaacttcacggctcgcctcggaccacagagcactttctccccctgttgtaaaaggaaaacaattggggaaaagttcgcagccaggaaagaagttgaaaacatccagccaagaagccagttaattcaaaaggaagaaaggggaaaaacaaaaaaaaacaacaaaaaaaggaaggtccaacgcaggccaaggagaagcagcagaggttgacttccttctggcgtccctaggagccccggaaagaagtgcctggcggcgcagggccgggcagcgtggtgccctggctgggtccggccgcggggcgcccgtcccgcccgcgcccgctggctctatgaatgagagtgcctggaaatgaacgtgcttttactgtaagcccggccggaggaattccattccctcagctcgtttgcataggggcggccggcggccaatcacaggcctttccggtatcagccagggcgcggctcgccgccgccggctcctggaattggcccgcgcgcccccgccgccgcgccgcgcgctactgtacgcagcccgggcggggagtcggaggccacccccgcgccccgcatccaagcctgcatgctggcccggggccccgcccgcgtgcggacccctttccgcagccacacgcaggcttgtgcggctccgcgagtggccacggtccggagacctggaaaaagaaagcaggccccgccggcccgaggaggacccggccggcgcgccgcacccggagaggcccggccccgcgagccgctgcaggcaggcgcagtggccgccacgaggctcccgaaccgggctgcagcccgcggacggccccagatcctgcgcggccgcccagggccaggcctccgcttccagggcgggggtgcgatttggccgcggggcccgggggagccactccgcgctcctgcaccgtccggctggcagctgcggcgaagcggcgctgattccttgcatgaggccggacggcgtccgcgcgtgccgtttgctctcagcgtcttcccttgggtcggtttctgtaatgggtgttttttaccgctgcgcccgggccgcggctcgatccctccgcgcgtctcacttgctgcgtgcgtcagcggccagcgaagagtttcctagtcaggaaagaccccaagaacgcgcggctggaaggaaagttgaaagcagccacgcggcttgctcccgggccttgtagcgccggcacccgcagcagccggacagcctgcccgggccccgcgtctcccctccggctccccggaagcggcccccgctcctctccccgcccccgtgcgctcgagcggccccaggtgcggaacccaccccggcttcgcgtgcgggcggccgcttccccctgcgccggtccccgcggtgctgcgggcattttcgcggagctcggagggccccgcccccggtccggcgtgcgctgccaactccgaccccgcccggcggggctccctcccagcggaggctgctcccgtcaccatgagtccctccacgccctccctgccgggccctgcacctcccggggcctctcatccaccccggggctgcaacccagtccccggatcccggccccgttccaccgcgggctgctttgtggtccccgcggagcccctcaattaagctccccggcgcgggggtccctcgccgacctcacggggcccctgacgcccgctcctccctcccccagggctagggtgctgtggccgctgccgcgcagggactgtccccgggcgttgccgcgggcccggacgcaggagggggccggggttgactggcgtggaggcctttcccgggcgggcccggactgcgcggagctgtcgggacgcgccgcgggctctggcggacgccagggggcagcagccgccctccctggacgccgcgcgcagtccccggagctcccggaacgcccccgacggcgcggggctgtgcggcccgcctcgtggccttcgggtcgcccgggaagaactagcgttcgaggataaaagacaggaagccgccccagagcccacttgagctggaacggccaaggcgcgtttccgaggttccaatatagagtcgcagccggccaggtggggactctcggaccaggcctccccgctgtgcggcccggtcggggtctcttcccgaagcccctgttcctggggcttgactcgggccgctcttggctatctgtgcttcaggagcccgggcttccggggggctaaggcgggcggcccgcggcctcaaccctctccgcctccgctccccctgggcactgccagcacccgagttcagttttgttttaatggacctggggtctcggaaagaaaacttactacatttttcttttaaaatgatttttttaagcctaattccagttgtaaatccccccctccccccgcccaaacgtccactttctaactctgtccctgagaagagtgcatcgcgcgcgcccgcccgcccgcaggggccgcagcgcctttgcctgcgggttcggacgcggcccgctctagaggcaagttctgggcaagggaaaccttttcgcctggtctccaatgcatttccccgagatcccacccagggctcctggggccacccccacgtgcatcccccggaacccccgagatgcgggagggagcacgagggtgtggcggctccaaaagtaggcttttgactccaggggaaatagcagactcgggtgatttgcccctcggaaaggtccagggaggctcctctgggtctcgggccgcttgcctaaaaccctaaaccccgcgacgggggctgcgagtcggactcgggctgcggtctcccaggagggagtcaagttcctttatcgagtaaggaaagttggtcccagccttgcatgcaccgagtttagccgtcagaggcagcgtcgtgggagctgctcagctaggagtttcaaccgataaa

SEQ ID NO: 75 - DNA, 453 bp, Homo sapiens
  source 1..453: mol_type = other DNA
ttcggaagtgagagttctctgagtcccgcacagagcgagtctctgtccccagcccccaaggcagctgccctggtgggtgagtcaggccaggcccggagacttcccgagagcgagggagggacagcagcgcctccatcacagggaagtgtccctgcgggaggccctggccctgattgggcgccggggcggagcggcctttgctctttgcgtggtcgcgggggtataacagcggcgcgcgtggctcgcagaccggggagacgggcgggcgcacagccggcgcggaggccccacagccccgccgggacccgaggccaagcgaggggctgccagtgtcccgggacccaccgcgtccgccccagccccgggtccccgcgcccaccccatggcgacggacgcggcgctacgccggcttctgaggctgcaccgcacggagatcgcggtggccgtggacag

SEQ ID NO: 76 - DNA, 560 bp, Homo sapiens
  source 1..560: mol_type = other DNA
acgcacactgggggtgtgatggaaagggggacgcgatggataggggtgggcgcacactgggggacgcgacggggaggggtgagcacacactgggggtgtgatggagagggcgacgcaatagggaggggtgggcgcacaccagggacgcgatgatggggacgggtgggcgcacaccaggtggcatgatggggaggagtgggtacacaccatggggggcgtgatggggaggcgtgggcgtacaccggggggcgcgatggggaggggtgggcgcacaccgggggacgcgatggaggcggtgggtgcacacggggcgcgatgggtgggagtaggtgcacactgagggcacgattggggagacacgaaggagaggggtgggcgcacactgggggacgcgatggccgggacacgatgcggagaagtgggtgaataccggggtcgcgatgggcgccctggaaggacggcagtgctgctcacaggggccaggcccctcagagcgcgccccttgggggtaaccccagacgcttgttcccgagccgactccgtgcactcgacacaggatc

SEQ ID NO: 77 - DNA, 157 bp, Homo sapiens
  source 1..157: mol_type = other DNA
ccacagggtggggtgcgcccacctgccctgtccatgtggccttgggcctgcgggggagagggaatcaggacccacagggcgagccccctccgtagcccgcggcaccgactggatctcagtgaacacccgtcagcccatccagaggctagaaggggga

SEQ ID NO: 78 - DNA, 114 bp, Homo sapiens
  source 1..114: mol_type = other DNA
ttgaggtctctgtgcatgcttgtgcgtaccctggactttgccgtgaggggtggccagtgctctgggtgcctttgccagacaactggtctgccgggccgagcattcatgctggtc

SEQ ID NO: 79 - DNA, 104 bp, Homo sapiens
  source 1..104: mol_type = other DNA
tgacgcgcccctctccccgcagctccacctggttgcgctcaacagccccctgtcaggcggcatgcggggcatccgcggggccgacttccagtgcttccagcagg

SEQ ID NO: 80 - DNA, 659 bp, Homo sapiens
  source 1..659: mol_type = other DNA
aacacactgtctcgcactaggtgctcgcggaagagcgcggcgtcgatgctgcggctcaggttgatgggcgatggcggccgcagatccagctcgctcagcgatggcgccggtcccacaccgttgcgggacagtcccgggccaccctggggtccgcgacccaacgacgcagccgagccccaggcgcctgaactgggcgtggccagctgcccactctccgccgggttgcggatgaggctcttgctgatgtccaagctgcctgcaccaacgttgctgggccctgcatagcagttattgggtcgctccggcacctcgctctttcctgacggcgccgggcacgccagacgcatcagcttagcccagcaagcgtgctccgtgggcggcctgggtctcgcggcagccaccgcggccaacgccagggcgagcgcccatgtcagctccaggaggcgcagccagaagtggacaccccaccaggcccacgagaagcggcccacgcggcctgggcccgggtacagccagagcgcagccgccagctgcaagccgctagccagcagccccagcgcgcccgccacagccaacagccgagggcccgggctggcatcccagccccgtgggccgtccagcaggcggcgacggcacaggcagagcgtgcccagagccac

SEQ ID NO: 81 - DNA, 813 bp, Homo sapiens
  source 1..813: mol_type = other DNA
gtctgcacgaagcccgcggcggcctgcagggggcccagcgactcgtccagggaaccggtgcgcaggagcagccgggggcgcggcgcgccggccgcccttgggggactctggggccgggggcgcagctcgatctgacgcttgggcactgtccggggcctggcgggcgcggcgccctcctccagagccacctccacacactcgaactgcgctggggcggcaggacttggcccacggggccgcagctctaggtaggtggcccagcgggagccaccatcggggacctgggactggcgtgggaccgcggcgggagacgctggccccggcggcaaggggctgatgaaggccggctccgtgaactgttgttgcgcctcgcgatcgtctgcgccggagcagccgaacaggggtccgacgccgaagatgacttccatctcccccgacggcagcgtgcgcagctggggctggggtggccgtgggccggaacctgggcctcgcgggaaacccgagccgggcccgtgccgctggcggctattctgggcgctgacggacaggcgaggctgcgcgcccgccccccgcccaggagccacccagggccaattcgctgggcctttcgcgtccggcccaacgtccgggggctccggagaacctggagccgtgtagtaggagcctgacgaaccggaggagtcctggcgccgcgcgggggccgtgggcagctgcctcgggatcccaggcagggctggcggggcgagcgcggtcagcatggtggggccggacgccgtgcactatctccctcgcattcgcctccgctggtggcgc

SEQ ID NO: 82 - DNA, 440 bp, Homo sapiens
  source 1..440: mol_type = other DNA
ctggagagaactatacgggctgtgggagtcaccgggcgactatcaccgggcctcctttccacatcctcctccgggaagggaccccgttccgggcctcgaccggcgcagactgggctgacccactttcttgggcccactgagtcacctcgaaacctccaggccggtagcggggaggagaggaggagcaggcgggggtgccaaggtgtgggctgcgccctggttagggggcgagcccggcttgtttatgaggaggagcgcggaggaggatccagacacacaggcttgcgcgcccagactcgcccggccagcggctggcggcctccgacgtcaccaaaccggttgggtgagagggcagagagcagggggaagggccgcagtcccgcccgcgccccccggcacgcaccgtacatcttgccctcgtctgacaggatgatcttccg

SEQ ID NO: 83 - DNA, 252 bp, Homo sapiens
  source 1..252: mol_type = other DNA
gagtgcggagtgaaggggtgcactgggcactcagcgcggcccttgggaggcagggccgccccagcctgccctcctgtctgggaaggccgtccagaagcaggagccccggggaaaacaactggctggacggggcggccttcagtgtctctcccagcctgagagtcgcttcccaccacctgggcacgaacctgctctgcgatctccggcaagttcctgcgcctcctgtcggtaaaatgcagatcgtggcgtctt

SEQ ID NO: 84 - DNA, 1539 bp, Homo sapiens
  source 1..1539: mol_type = other DNA
tcttctttccgcccctagggggcacaagcgggcatgtccaagcgcctaggagcccgtaccgctggggacctccccttccgcgaaccccgagcgggtagacccagagcaatccgagtgtggaaacaatggagagggggcgtgttgagctggggtctccatgcctcgttggggagagggaggtgagtttgtgtcttctggaaggcgtgggggctgtgccctcgtgggggtaggaagtgctcccgtggggcggggtgcggatcggagaggtgagtgggtgcgtctgtccagcggtccgcccggtgtggtcgtgcccggcccgcgtggggatgggggtgtctctcccgctgggcaactataccagcgcaaccggggcgtcggcgcggcccacgctagcggcgctgctccggcggcgggggctgggcgtggcggtgatgctgggcgtggtggccgcgctgggcgtggtggccgcgctgccgccctcacccgggcagccgtgctggagaaggatgtcggcgcacagctggcttccagcctggcgggcgtagaacagcgccgtgcggccctgggcgtcacgggccgccacgtccgcgccgtactagagggcggaaacggccgcgtgaccgcgcgtccccagggcgcccacacccggcgccgcctcccccacatggccaagcctacttccggggtccctctgggaatttcgggctttcccgcgccaggcgttttccgagatgaagcctcaaagaccccctttcctccccccagctcacgtacccacagcagcagttgcgtgatgacgacgtgggcgagctcggccgccaggtggagtggggagcgcagctgtgggtcctctacgctggtgtcgagcggcccgtgtcgcgcatgggccaaaagcaggagaacggtagccacgtcctgggcctgcacggcggcccacagctggcggcccagcggctcctccgaggtgctcagcggcgccaggaacagtagctgctcgtacttggcgcgaatccacgactcgcgctcctccctgcaagaccagggatcaacggaaaaggctctagggacccccagccaggacttctgcccctacccacgggaccgtctcaggttcgcacaccctcagcaaccctccccccgctctgttccctcacgcttaccgcgaagagtcccgcgagggcttggcacggcctcgcgtgtcgctttcccacacgcggttggccgtgtcgttgccaatagccgtcagcaccagggtcagctcccgtggccagtcgtccaagtccagcgagcgaacgcgggacaggtgtgtgcccaggttgcggtggatgccagaacactcgatgcagatgagggcgcccaggttcaagctggcccacgtggggtctgcggaaggagcgtagaggtcggctcccagccgggcagcacaggcaccccggcattcactacactccctagcccctccgctgcctcctggcactcactgggggccccgcagtccacgcagattgaattccccttggcgttccggatcgcctggat

SEQ ID NO: 85 - DNA, 2648 bp, Homo sapiens
  source 1..2648: mol_type = other DNA
agccaggtccagcccccgcgcctgacaccggccggacgttcccggggcgccgcagctgcggcgggaactctgggatccggagccatctgctcccacccgctccggagccaaaccccgggggccgcctccgctcccggacccgcctcctctcccgggagtgtgagccgaaccaagagtctcctgcctatctcctccagtaggaaaatagtaataataatagacaccctgcccccgtaaaaaacactaccttccccgtaccgcctcccaagtctcccggggtacggattgcctttgcagcagttccgccccacctgactcactccagggtcagccccgggtgggtttcaatgcggctctggggagggggtgggcagtgggggaagtgaggcttcctatccgccccctctcacttcacatttaaatattctgcacgttccagcccccgcggactcgcgtaccgcccaatccgccttcaccgcacgaaaaacatcactagcctgctctcagcccaggggacgactagtccctggcgagaagctgcctgcaaggtcactgtcatgccacctgccccaagtgctcaggggaaactgaggcttcctcatccccttcaccttcaacgtcgctctaaacacggcaaagccccgtttccatgctcccagagttcagctgaggctggaagtggggtcctgggcttctctgggagcaattttctagtcactctgatcaaggacgttactttcccagaaagctctgaggctgagtccctctgaaatcaagtcctttctcctgtcgcacaatgtagctactcgccccgcttcaggactcctattctttgccccaatccttgacagaggggtgagcttggttcatccgcccaccccagagaaaagcttccctagtttcctggacctcgctcctccaccccaagctgagcattccaggtacccttccctccctgttctcaagccctgactcaactcactaggggaagcgcggagctcggcgcccagcagctccctggacccgctgccagaagacaggctggggggtccgggaaggggcccggagccaggaggccctcctgtgctcttggtgaagatgccgctgataaacttgagcatcttgcggtcacgagtggatgctcggcccccctcccggccccgtttcagccccggagctggaggctccagagtgattggaggtgcaggcccggggggctgcgcggaagcagcggtgacagcagtggctggactcggagttggtgggagggttagcggaggaggagagccggcaggcggtcccggatgcaagtcactgttgtccaaggtcttactcttgcctttccgaggggacaacttccctcgggctccagccccagccccgaccccaccagaggtcgaagctgtagagccccctcccccggcggcggcggcggtggcggcggcagagaccgaagctccagtcccggcgctgctctttgaccccttgaccctgggcttgccctcgctttcgggccatgacaggcggctacccgcgcccttgcccccgccggctttggctccactcgtggtcacggtcttgcaaggcttgggagccggcggaggaggcgccaccttgagcctccggctgccggtgccagggtgcggagaggatgagccagggatgccgccgcccgcccggccttcgggctccgggccgccccagctcgggctgctgagcagggggcgccgggaggaggtgggggcgcccccaggcttggggtcggggctcagtcccccggagagcgggggtcccggagggacggcccagagggagaggcggcggccgggagcgggggagactgggcgggccggactggccggagccggggacagggctgggggctccgcgcccccggtgcccgcgctgctcgtgctgatccacagcgcatcctgccggtggaagagacgttcgtgccgcttcttgcccggctcctccgcgcctcgggggctgccaggatccccagtctcggagcctctggcaccggcggcgccggccgcggccgcagacggagaaggcggcggcggaggcaccgactcgagcttaaccagggtcagcgagatgaggtaggtcgttgtccggcgctgaagcgcgcccgcgccccggctcatggggcccggagacccccgagctggggaggggaggggactcccccggactgcctcaggggggcccggccatggggccgccctgctcgctgcccccagcccccggaccccgctgagcccccggcccggctccgctgtcgccgccgcctccgccgcctccgcttgcgcccccctcccatcacatggggcgccccctccccatgctccccgccctgcgcccccaccctcttggagccccgggaccttggtgctgctccagggaggcgcgccggaccgtccaccccggcctgggtgggggcgctgagatgggtgggggagggcggggaggacagtagtgggggcaaatgggggagagagaggaaaagggagcagaaaaggggaccggaggctaggggaaacgaacctgtgcgggggaggcaggggcggggaattgggactcaagggacaggggccgcggatgcggtcggaaagagggtctagaggagggtgggaagctagtgg

SEQ ID NO: 86 - DNA, 452 bp, Homo sapiens
  source 1..452: mol_type = other DNA
aggagcgcaaggcttgcagggcatgctgggagagcgcagggaacgctgggagagcgcgggaaatactgggattggctcccgagggctgtgaggagggcacgaggggacactccgatgaaggcagggcacgcggggcgagccgggagcgtctcctgagggcagcgaggagggagctgaggcacgcgggctctcaatcgacgccccacagagaccaagaggcctggccttggggggcagctgcttgaaggaggcagagcggaagcgagggagactgctggaggccctgccgcccacccgccctttcctccccctgaggagacgcctgacgcatctgcagtgcaggaggccgtgggcgttagaagtgttgcttttccagtttgtaagaccattttcctgattctcttccccacggttgcggaggagcaggtcagggccgccatgagggcaggatc

SEQ ID NO: 87 - DNA, 232 bp, Homo sapiens
  source 1..232: mol_type = other DNA
tcgaccgctactattatgaaaacagcgaccagcccattgacttaaccaagtccaagaacaagccgctggtgtccagcgtggctgattcggtggcatcacctctgcgggagagcgcactcatggacatctccgacatggtgaaaaacctcacaggccgcctgacgcccaagtcctccacgccctccacagtttcagagaagtccgatgctgatggcagcagctttgaggaggc

SEQ ID NO: 88 - DNA, 221 bp, Homo sapiens
  source 1..221: mol_type = other DNA
tgtgccgtcgcacacagacgccctcaacgtcggagagctgtgagcggggccgtgctcttgggatgggagcccccgggagagctgcccgccaacaccactccgacgtgatccatgctggacataaagtgctcttccctccgctagtcatcggccgagcgggcccctcgctcctgggtgtaagttctttctgtgcgtccttctcccatctccgtgcagttcag

SEQ ID NO: 89 - DNA, 477 bp, Homo sapiens
  source 1..477: mol_type = other DNA
ccatgcgccgctgcgcgcgcgagttcgggctgctgctgctgttcctctgcgtggccatggcgctcttcgcgccactggtgcacctggccgagcgcgagctgggcgcgcgccgcgacttctccagcgtgcccgccagctattggtgggccgtcatctccatgaccaccgtgggctacggcgacatggtcccgcgcagcctgcccgggcaggtggtggcgctcagcagcatcctcagcggcatcctgctcatggccttcccggtcacctccatcttccacaccttttcgcgctcctactccgagctcaaggagcagcagcagcgcgcggccagccccgagccggccctgcaggaggacagcacgcactcggccacagccaccgaggacagctcgcagggccccgacagcgcgggcctggccgacgactccgcggatgcgctgtgggtgcgggcagggcgctgacgcctgcgccgcccac

SEQ ID NO: 90 - DNA, 1200 bp, Homo sapiens
  source 1..1200: mol_type = other DNA
gtcctaacatcccaggtggcggcgcgctggctccctggagcggggcgggacgcggccgcgcggactcacgtgcacaaccgcgcgggacggggccacgcggactcacgtgcacaaccgcgggaccccagcgccagcgggaccccagcgccagcgggaccccagcgccagcgggaccccagcgccagcgggaccccagcgccagcgggaccccagcgccagcgggaccccagcgccagcgggtctgtggcccagtggagcgagtggagcgctggcgacctgagcggagactgcgccctggacgccccagcctagacgtcaagttacagcccgcgcagcagcagcaaaggggaaggggcaggagccgggcacagttggatccggaggtcgtgacccaggggaaagcgtgggcggtcgacccagggcagctgcggcggcgaggcaggtgggctccttgctccctggagccgcccctccccacacctgccctcggcgcccccagcagttttcaccttggccctccgcggtcactgcgggattcggcgttgccgccagcccagtggggagtgaattagcgccctccttcgtcctcggcccttccgacggcacgaggaactcctgtcctgccccacagaccttcggcctccgccgagtgcggtactggagcctgccccgccagggccctggaatcagagaaagtcgctctttggccacctgaagcgtcggatccctacagtgcctcccagcctgggcgggagcggcggctgcgtcgctgaaggttggggtccttggtgcgaaagggaggcagctgcagcctcagccccaccccagaagcggccttcgcatcgctgcggtgggcgttctcgggcttcgacttcgccagcgccgcggggcagaggcacctggagctcgcagggcccagacctgggttggaaaagcttcgctgactgcaggcaagcgtccgggaggggcggccaggcgaagccccggcgctttaccacacacttccgggtcccatgccagttgcatccgcggtattgggcaggaaatggcagggctgaggccgaccctaggagtataagggagccctccatttcctgcccacatttgtcacctccagttttgcaacctatcccagacacacagaaagcaagcaggactggtggggagacggagcttaacaggaatattttccagcagtga

SEQ ID NO: 91 - DNA, 900 bp, Homo sapiens
  source 1..900: mol_type = other DNA
caccttccccgaggtaattattttctggggggtaggggtgggggttgggagggtgaagaaaggaagaaaaagaaggccgatcacactgggcaccggcggaggaagcgtggagtccattgatctaggtacttgtggggaggggagaacccgagcagcagctgcaaacggaagggctgtgagcgagcgggcgggcgggtggctggcagcgaggccaccagcagggggggcccgggccgaggccgcgccacctcggcaccacgcgggcagccggtgcggcggggtcgccacggccaggggagcgctgggtgcccaccatggcagttatgcaagcggtgaccccctggtcttgcctccccgccgccctgcactccttcctccccgctgccgacacttggatctctctagctctttctctcccctgtgttttcaaacaggaagtgcacggctgtctataacgtgctgccgggtctcaggatggaggagtgaagtctcctgtcgccgtggttccagcctccggagctcgcccaagccgcgtccccagagagcgccctgagagaacagggtggccgcttggtccaggtgcgcggggtcgggtctgggtccagggagcgggtcgggaagtctgcggcacggagcactgctagtgtcggatctgcatctccagctctgtgctgcagcttcacttgcccgccccccaccactggcttctcacccggggtctctgccaaactctggctgctgccgccctgggttcgggccggcggaaggccctgggcgtgcgctgcggagccgcctgcgaggactccactagggcgctttccaggctggactgccccgggctgcgctggagctgccagtgctcggggagtcttcctggagtccccagctgccctctccacc

SEQ ID NO: 92 - DNA, 200 bp, Homo sapiens
  source 1..200: mol_type = other DNA
ctcttcccaagttacgccaccggtcgaggacggcaggagacccccgagtgcagagaaagctcaaaccggcagcgaagtcggtcctagccaagctgaaaaaacgtctcggatttcgcggacagcggcctagacacagcccgatcttccagtcctagtgccctggtcgagacggttctatccttttgcaaagaagccggaaa

SEQ ID NO: 93 - DNA, 400 bp, Homo sapiens
  source 1..400: mol_type = other DNA
tctcggttgcaatccccaccctcctcacccagcagggcaggaggcacccaacttggaggagaaaggggtgggggaggtgaaacagagaccggagagtcacgagggctgggccgccgagagcaggagaatataccgtgtcacacacctccattctctcacacacgttgcagacacaaatcactgacggtttccacgtgctgcgctcgtgagcggaggtgttcaaagagggggcagatgagttacttcccgagacggaaccgggggtcccacgtccgccgccttcagtagcacaaccaatctctgaacactcaaaccgcgcatctctggcgcatcaccatcctatttaaggccacgggctccgcccttttcctcccctcccttcttttccactctttttcca

SEQ ID NO: 94 - DNA, 700 bp, Homo sapiens
  source 1..700: mol_type = other DNA
ctgccagagatgtgtctgtcttgcgccccgcatgcactgcctgcggggctgcgctgcactccccggcggcgccacgggtctggcccccgcgcttctacgtgttggggggatgcatggaccttggagatccgtagttggccctaaccttctcggaatctcctctgcacgcgctgcctgttcctcctctgcacgctctgtccgttcctttgcaacttctgtgggaattgtcctggcgtgggaaacgcccccgcgctctttggcacttagggtgtgagtgttgcgccccttgccgcagcgctcagggcagcatcccgctcgaggatgcagggttctcaccaagcagtgagggggactcacgcgccgccggggagcggagccaggctccgagaagggagcaggctcgagccgctgggttttcgcaagccttggggcctctggccgcccttccatgcctccgggcgcgggcggctcagcaggtccccggcttcgggaagttttgtgcgcggatcgctggtggggagggcgcgcgggccagtggctgagcttgcagcgaagtttccgtgaaggaaactgcatgtgcctttggaggcgactcgggactgctgtagggtggactgggtgtctatggagttgcgggtcagagcgagtagggtgggtcctttcctgggacaggactgggaattggggctcgaagtagggg

SEQ ID NO: 95 - DNA, 200 bp, Homo sapiens
  source 1..200: mol_type = other DNA
aggggtgtcctccaacatctctgaaccgccttcccttcctcctcactggcgccctcttgcctcagtcgtcggagatggagaggcggctgaagattggcaggcggcggccagggtcgaggctgggagactcagagccgctgaggctgccggagctcagggagccgcttaggtagctgtcgcggtccgacagcgagtccggg

SEQ ID NO: 96 - DNA, 5000 bp, Homo sapiens
  source 1..5000: mol_type = other DNA
tctgactctcgggctggagcagccgagacagcgctccccagcgggactacagaatcccgggtgtcggcctgggggccctggattggcagtggtggagtcttctgagcctaacagctactaggaatgacagagttgcagatggctttgtcgcccgcggggcggctcaagcgtcctgggtcccaggcctctgtcctacggccaggccgccggctcaacgggccgaagggaatcgggctgaccagtcctaaggtcccacgctcccctgacctcagggcccagagcctcgcattaccccgagcagtgcgttggttactctccctggaaagccgcccccgccggggcaagtgggagttgctgcactgcggtctttggaggcctaggtcgcccagagtaggcggagccctgtatccctcctggagccggcctgcggtgaggtcggtacccagtacttagggagggaggacgcgcttggtgctcagggtaggctgggccgctgctagctcttgatttagtctcatgtccgcctttgtgccggcctctccgatttgtgggtccttccaagaaagagtcctctagggcagctagggtcgtctcttgggtctggcgaggcggcaggccttcttcggacctatccccagaggtgtaacggagactttctccactgcagggcggcctggggcgggcatctgccaggcgagggagctgccctgccgccgagattgtggggaaacggcgtggaagacaccccatcggagggcacccaatctgcctctgcactcgattccatcctgcaacccaggagaaaccatttccgagttccagccgcagaggcacccgcggagttgccaaaagagactcccgcgaggtcgctcggaaccttgaccctgacacctggacgcgaggtctttcaggaccagtctcggctcggtagcctggtccccgaccaccgcgaccaggagttccttcttcccttcctgctcaccagccggccgccggcagcggctccaggaaggagcaccaacccgcgctgggggcggaggttcaggcggcaggaatggagaggctgatcctcctctagccccggcgcattcacttaggtgcgggagccctgaggttcagcctgactttcccgactccgccgggcgcttggtgggctcctgggcttctgggctcacccttacacctgtgtactaaagggctgctaccctcccgaggtgtacgtccgccgcctcggcgctcatcggggtgttttttcaccctctcgcggtgcacgctttttctctcacgtcagctcacatctttcagtacacagccactgggtctccctgcccctccagcctttcctaggcagctttgagggcccagacgactgaagtcttactgctaggatgggaacacgatgaaaaaggaaggggcccagtcaaaagtcctctcctcttcggtttttcttcaactgtccttcacaaaaacatttatttctgtcccagcgccctggcggatttcggcagatgggccctagggggttgtggaggccaaattcccaggatgctggtcctgcctttttcattggccaaaactgtatttcctacaacgactaaagataaccaagaactgagtagaccctgttctctcaccagatctccctggctctgtttaacttttcctggtgcaatgcgatggcaccaccagctccccaggcaggcaccactccctcaagataccatttggggtagggatttgagtcctggagagggtcagcggggcgccggggtgggggtgggaaggagactgacagggacacaccgcgagctccgcatactctcctctgccccctgtagcccggggctttaatgaccccaagcagatttcctgtctctggtctagccagctgcccctagggctggattttatttcttcatggggtttcaccctaaagggccccctggtcatgggacctggttgggaacaaatgaaagatgtcttgtagcaaatgctttcaggggagcagaaaagaagattgggcacttccagtcacttggtcactttaggtggctggaacaaaactggtgactttcacgactgctacagggtgagggggtgaagggtggcagagaggtgacaagccactgggaatcctattcagtggggatgccgacagggagtggctgtaatcaactgagcaacatctgtgtgaatgttattcacaggtcaggacagcagcttggtcttcccaggtgaggaactgaggactggcctgcatagatttgtgcagtaggtgagtagcttccaaatttattttcagaacttccatgtagtacctgcctctccatttaaatattttttaaaattttatttatttaaatattttcttggttagctttccaagagggaggaaaagaggggagttgcaacaagtagtgcccctatgctgggattcattttccagagtaaagcctgggactggcaccctgacccctaccggcaggtgaaaactccaggcaaactgctgagatcccacctgggctggctgagatagtgcctggggtgcatccctcagcagctgccacctgggccctggggccatctctttctctggcatcaagcagccaggtgtcaaggccttcccagcaatccatgctgcatggctgggtcttgttctagcaggtcgatgggcagggactggtagcttagccagggcaccagtgcgtggctgtgggtttgtgtgcttctgtggagaagcatgatgtgtatgtgtgtgtgtgggcacaggcatgaggaagggttcatttgtgcaggtatctcccatgtatatcagtgtgggagagtgcctgaggatgtgtttgtgtgtctgaaaatgggcggagggtctgttgtgctaatgtgtgcaggggtgaacatgtgtgtgacagtctgtgtgtttccctgagtggtggctgcgtgagagggtgaggggatttggtgttgtctaccatgcccggcacatagcaggctcttaataatcttgaatttaattaatgttaaatgtgtatgttcccatccttgtggaagttggtatagagcctgttttcctgtgattgtgagactggaaaatgggggacgggcaggggcgagacaggatacagaggctactgttttcttcctccctagaagtaagtacatagaagagtgggctctggcacctcacgggacatcaccaagtcctgtgtggctggctaggctgtcccaaggtggcttcaggcatcacttgaatcttttgagaccttcaggcagtagcctgccattcaccctgtcagtcagcagaagttgggcccacacaggccatagaaacacagagcagttcccgggaggacctgagctgtccctgagagcagagcttccaggagaggccgcaggaactgccttgaccggaattcctcttggggtgcaaaggtggagggacacatggtgcgaccccaggcagaggactgcagccactccgtgcagtcccagcctctggggtagccccttgacctccaggcctgcacagatccaaggccgaggtccaggctccagcgccaaattagctggcctagcagcctgcagccgctctaatctcaactaggaaggaatccttgcgcttagaaagtccaagcgaaagggtattctgattttatcccggttttaccagaaaatgctgaaaggaaaagccccgagaggacacagtgctctaggaactcggggcgccacgagcgcctcatcccctcccttccgcccggccgcggtgccctggtcgctgagggacgcggtcagtacctaccgccactgcgacccgagaagggaaagcctcaacttcttcctctcggagtcctgcccactacggatctgcctggactggttcagatgcgtcgtttaaaggggggggctggcactccagagaggagggggcgctgcaggttaattgatagccacggaagcacctaggcgccccatgcgcggagccggagccgccagctcagtctgacccctgtcttttctctcctcttccctctcccacccctcactccgggaaagcgagggccgaggtaggggcagatagatcaccagacaggcggagaaggacaggagtacagatggagggaccaggacacagaatgcaaaagactggcaggtgagaagaagggagaaacagagggagagagaaagggagaaacagagcagaggcggccgccggcccggccgccctgagtccgatttccctccttccctgacccttcagtttcactgcaaatccacagaagcaggtttgcgagctcgaatacctttgctccactgccacacgcagcaccgggactgggcgtctggagcttaagtctgggggtctgagcctgggaccggcaaatccgcgcagcgcatcgcgcccagtctcggagactgcaaccaccgccaaggagtacgcgcggcaggaaacttctgcggcccaatttcttccccagctttggcatctccgaaggcacgtacccgccctcggcacaagctctctcgtcttccacttcgacctcgaggtggagaaagaggctggcaagggctgtgcgcgtcgctggtgtggggagggcagcaggctgcccctccccgcttctgcagcgagttttcccagccaggaaaagggagggagctgtttcaggaatttcagtgccttcacctagcgactgacacaagtcgtgtgtataggaaggcgtctggctgtttcgggactcaccagagagcatcgccaaccagaacggcccacccggggtgtcgagtcttggtagggaaatcagacacagctgcactcccggcccgcgggccttgtggcatataaccatttatatatttatgatttctaattttattataaaataaaagcagaaatatttcccgaagaacattcacatgagggcattacggggagacggcaagtcggcggctcggggggcgcgctcagccgggagcgctgtagtcacagtcccgggaggaagagcgcg

SEQ ID NO: 97 - DNA, 1500 bp, Homo sapiens
  source 1..1500: mol_type = other DNA
tggaacaagtgtcagagagtaagcaaacgactttctgagctgtgactctgctcctcgactgcccacgtgctctccgctgtctgcactcctgcctcacctgggctgactcggactctccacctcctttgctgcttccggcatgagctacccaggagcctaaggcgctccttcccgcaactccggtccccgcgccccgggactgcaaatcctttaaacagaggccccagagctaggggttttcccaggctctggtgggcgtgggctgacagtcgctgggagccccgcaacaggggggatgtccaggcaggtatgcacccagctcccggcgtttcccggagtcaccacaatgtttccctttctctctcccccacgtatgctgctaggggtactccccagataggattttctttgtcttttctcctagtaacaccgaagccctctcgtgcccggggactgcagaggaacgccagaccatccggaccttgcgggatggctcggtgtgtgtgttttactgtgtgtcggagtgtcgcgcatgtgtgcgtgttggggcgcgttatcaacaggggcctagggcacccccactctttcttgctctcttcccccatcacttcatggacctccgaggcgcaaagcgctcgaccctctcctgggctcagtggcttgggtactccgggctgagctcagctggggagtccccttacccagcccgcaccggcaccccgaagcttcaaagttgcggcaaacagttgcggggagcagaggaactgaggtccaggccagcgcgcccgcggtcgctcgccttggggagcaggctgagccgagggtcgtgcgggtgcgcggcagaggcggtaggaggcggaggagaggggggagaaagagggggcggtggggaacagctgccggggtaggcgaggcgcaaggtggctccccgcggccccgcgccccgcggctctcggacgcaccaggcagccaatggctgcgcagaggtgtacagcagatggcgtctgactgcgccgttccttcctcctcctcctcctcctccttctcttcctcctcctccttctcttcctcctcctcctccttcagtgctgaggagccagagtcgccgccgggttgccagacgctggaatgggtggtcttccgacacacaccaccatctttcttgcgctcgggaagctcggggctcagcggctcccagaggttacggcggcggctctggcgagacgggtgagtgcaagcacgcggagccccgagtcggggatgccgggccccctggccggccgactggggcgcggggtggcagcgccggggaagggggcgcgctgccggcgcagactttgctctttcctcgccggacagccatcgtcgccccttctcccagccagacgcgggaacttggaagcggatcttctcggacgcctctggcttggggctgcgggaagcgtgggctgcccggggcgcagtgtgcggagaccctctaggcgggcggggacgccccac

SEQ ID NO: 98 - DNA, 800 bp, Homo sapiens
  source 1..800: mol_type = other DNA
gttattatccacggggtcctaattaaagcttgattaaaatgcccttctttctctaaaaaattacgaactaggcaacttcatacattttgaatggcgcagtgtttcctcttccaactgtttagtttgtagtatactatgtaagcaacatcaattatcaacccttgcaagatgacaacatgagcctgtgggggaagcacttgaggggagggaggagaaacttctcttttttaataatcagccggaaacaatgtttaacaagaatctgatgaggtcactgcagtaaatatttttcctcttacagagccaatcatcacggagggatcccctgaatttaaagtcctggaggatgcatggactgtggtctccctagacaatcaaaggtgtttgctttctgctctgttgcttttaaattgtatgggaaaggaagattggtccgacggcgcgcttgtggcccggccggagcttgcgtgcgcgttctgacggctgggtgctgtgttacaggtcggcgcagttcgagcacacggttctgatcacgtcgaggggcgcgcagatcctgaccaaactaccccatgaggcctgaggagccgcccgaaggtcgcggtgacctggtgcctttttaaataaattgctgaaatttggctggagaacttttagaagaaacagggaaatgaccggtggtgcggtaacctgcgtggctcctgatagcgtttggaagaacgcgggggagactgaagagcaactgggaactcggatctgaagccctgctggggtcgcgcggctttggaaaaacaaatcctggc

SEQ ID NO: 99 - DNA, 40 bp, Homo sapiens
  source 1..40: mol_type = other DNA
tccctgctgtgggacccgaggagaggagaactggttcgct

SEQ ID NO: 100 - DNA, 500 bp, Homo sapiens
  source 1..500: mol_type = other DNA
tctctctctctctcttgcttggtttctgtaatgaggaagttctccgcagctcagtttcctttccctcactgagcgcctgaaacaggaagtcagtcagttaagctggtggcagcagccgaggccaccaagaggcaacgggcggcaggttgcagtggaggggcctccgctcccctcggtggtgtgtgggtcctgggggtgcctgccggcccggccgaggaggcccacgcccaccatggtcccctgctggaaccatggcaacatcacccgctccaaggcggaggagctgctttccaggacaggcaaggacgggagcttcctcgtgcgtgccagcgagtccatctcccgggcatacgcgctctgcgtgctgtgagtacaacctgctccctccccgggcacagatatgacagaggggcttagagggggcccagctttgagatgggttgttcttatgtcacaggacagagtgatctgacatgcacacttccccgccaccctgtcat

SEQ ID NO: 101 - DNA, 500 bp, Homo sapiens
  source 1..500: mol_type = other DNA
tgtcctcgaagaagggcctgagcagcagcagaggaccccaggcgaccgtgcctgagccgggcgccgacgacgactgagcacctgatatgtccccggcactcgcagccccgcggccggagtcgctgtgggtgagcggtcgtcgagcttcacagaggccgggctctgtgccagggccccgacagggcaggaagcagatagagtcccacaagcacaagcccagtgcgcagaaagggttacttaaaaaataagttctgtgataaaatcaaacagggtgaagggctggaaacaggtcatgagggcgcaaacaggtcgtgagggcgcaaacaggtcgtgagggcgcaaacaggtcgtgagggcgcaaacaggtcgtgagggcgcaaacaggtcgtgagggcgcaaacagatcgtgagggcgcaaacaggtcgtgagggcgcaaacaggtcgtgagggtgcaaacaggtcgtgagggcgcaaacaggtcgtgagggtgcaaacaggt

SEQ ID NO: 102 - DNA, 300 bp, Homo sapiens
  source 1..300: mol_type = other DNA
aaatgagacctctggggagactgtcaaccccaggggtaaaacaaaaattctgatcagaaactgagtttcccaaagaaggggctaaatgttttccaacactttcggggctcagggaagatgactctgtaaggacactgagaatcttcctcgcgtgccacggggaggaggactgggggcgtttgaggggctcagcgcaccagaggagtgaggtggaggagggcgttcccgcgtcctcctcttcaatccagagcagctcaacgacgtggctccctttctatgtatccctcaaagccttcgcgt

SEQ ID NO: 103 - DNA, 600 bp, Homo sapiens
  source 1..600: mol_type = other DNA
taggctctagtggacctagcagtgggagagctacttgggctggtttctttcctgacgctgcagggatgggcatcggcctggaaccagaagcgcaggagctgggccacggcagagtaattaagaaaataatgaaattgatggcggatgggggcgctagaaatcctggggcgtctacttaaaaccagagattcgcggtcggccccacggaatcccggctctgtgtgcgcccaggttccggggcttgggcgttgccggttctcacactaggaaggagcctgaagtcagaaaagatggggcctcgttactcactttctagcccagcccctggccctgggtcccgcagagccgtcatcgcaggctcctgcccagcctctggggtcgggtgagcaaggtgttctcttcggaagcgggaagggctgcgggtcggggacgtcccttggctgccacccctgattctgcatccttttcgctcgaatccctgcgctaggcatcctccccgatcccccaaaagcccaagcactgggtctgggttgaggaagggaacgggtgcccaggccggacagaggctgaaaggaggcctcaaggttcctctttgctaca

SEQ ID NO: 104 - DNA, 800 bp, Homo sapiens
  source 1..800: mol_type = other DNA
gaggttgctgactcaggagccaggagctgagaaactcctaggctagcagccgttgagcctaattttattttctggctttctccgaaatgtctcgtttccctcatctttctggtccttttcgtctctcttattttccccaaaacgtctacctcacttcgtcttcctttctcctcccctccccctctctttcctctatactctcttcccatttagccttgcaggcccctcctccccggtgttggagagctcaaagacgcgcgaaactcaaggatctggccctgaccagggacgggattaggcgggaagtggtgacggcctgaaaaggctgggctcgaacccgtgccttcctgaaaggactctccccgccacaagtcacacccacccgcaggcctgctggccaaagaaacaaaggagtcgggcgtggatccaggagaaacaggttttcgctctcggatctccctgggcaaatcagggatcctgagcgctataccccgcagtcgtacggagcctctgggaaaggggatttaagggtgacttccactttcagcttcggctacttgttgcctgcggtccaagccttctctgcttcctcctacctcgtcttaggcctctgtagaaagtgcacgccgcgtttccccttccaggctctgagagggcctgcaggcccgtggccgcctccgacaagatgccttccagtgctaggggggccactttggcgggatgggggtcggttggttaaaaaaaacttaagttctggctcagtcgagtgtggcaaaagccgagggtcgggggttgggggg

SEQ ID NO: 105 - DNA, 2000 bp, Homo sapiens
  source 1..2000: mol_type = other DNA
tactgacctggtctccgcctcaccggcctcttgcggccgctgcagaagcgcactttgctgaacaccccgaggacgtgcctctcgcacagggagcgcccgtctttgctggggctggagcggcgcttggaggccgacactcggtcgctgttggactccctcgcctgccgcttctgccggatcaaggagctggctatcgccgcagccatagctgctcagcgagggcctcaggccccagcctctactgcgccctccggcttgcgctccgccggggcgagggcaggacctgggcggccagggaaagggcagtcgcggggaggcagtgctaaaatttgaggaggctgcagtatcgaaaacccggcgctcacaaggttagtcaaagtctgggcagtggcgacaaaatgtgtgaaaatccagatgtaaacttccccaacctctggcggccggggggcggggcggggcggtcccaggccctcttgcgaagtagacgtttgcaccccaaacttgcaccccaaggcgatcggcgtccaaggggcagtggggagtttagtcacactgcgttcggggtaccaagtggaaggggaagaacgatgcccaaaataacaagacgtgcctctgttggagaggcgcaagcgttgtaaggtgtccaaagtatacctacacatacatacatagaaaacccgtttacaaagcagagtctggacccaggcgggtagcgcgcccccggtagaaaatactaaaaagtgaataaaacgttcctttagaaaacaagccaccaaccgcacgagagaaggagaggaaggcagcaatttaactccctgcggcccgcggttctgaagattaggaggtccgtcccagcagggtgaggtctacagaatgcatcgcgccggctgcggctttccaggggccggccacccgagttctggaattccgagaggcgcgaagtgggagcggttacccggagtctgggtaggggcgcggggcgggggcagctgtttccagctgcggtgagagcaactcccggccagcagcactgcaaagagagcgggaggcgagggaggggggagggcgcgagggagggagggagatcctcgagggccaagcacccctcggggagaaaccagcgagaggcgatctgcggggtcccaagagtgggcgctctttctctttccgcttgctttccggcacgagacgggcacagttggtgattatttagggaatcctaaatctggaatgactcagtagtttaaataagccccctcaaaaggcagcgatgccgaaggtgtcctctccagctcggcgcccacacgcctttaactggagctccccgccatggtccacccggggccgccgcaccgagctggtctccgcacaggctcagagggagcgagggaagggagggaaggaaggggcgccctggcgggctcgggatcaggtcatcgccgcgctgctgcccgtgccccctaggctcgcgcgccccggcagtcagcagctcacaggcagcagatcagatggggattacccgccggacgcaaggccgatcactcagtcccgcgccgcccatcccggccgaggaaggaagtgacccgcgcgctgcgaatacccgcgcgtccgctcgggtggggcgggggctggctgcaggcgatgttggctcgcggcggctgaggctcctggccggagctgcccaccatggtctggcgccaggggcgcaggcggggcccctaggcctcctggggctacctcgcgaggcagccgagggcgcaacccgggcgcttggggccggaggcggaatcaggggccggggccaggaggcaggtgcaggcggctgccaactcgcccaacttgctgcgcgggtggccgctcagagccgcgggcttgcggggcgccccccgccgccgcgccgccgcctccccaggcccgggagggggcgctcagggtggagtcccattcatgggctgaggctctgggcgcgcggagccgccgccgcccctccggctggctca

SEQ ID NO: 106 - DNA, 800 bp, Homo sapiens
  source 1..800: mol_type = other DNA
gggggacacagagaggaggggttgcgggcctgtgagaatgaagagcacagagcggagagggggaggaggagggaaaggaaggcgtggcagtgagagagaagaggaagaagagaggaggagtggggaggggagggagagcaagacagcagcgggtctggattcccctccgagccacatctggtcaggttctaagtaattagaagattttcccattggtttacccaagggctctctctctgattaattttcgaaagagttggccaattttaatcatagcaaacacgatgatcacggtgatcatggcctgaacagctaaaagcagaaaataaaacccccagaacggactatgatcttgacctttgcccgtggtcaccggctgggcccacacccagggttctgagctgttgggagccaaggctgggtggacaggggcttccgaggagctgtccgcagcggggcggggaggcgggccccgggggcccgggcactccgcgtcaccccccggcagggcccagagcggcaggccggcgtgcgccccagggcctgcgcaccgtgggggctcttccccgcccacgaggcctaggtgctgccgcagccaccccaggaagggccccaggccacagtcgcagcgccaggagttgtgccccaacaggacctccgtcagccggggcagagccccaaacacgtcgccaggcagggtctccagctggttgtggtcgagctggacgctctccaggctgctgagattgcggaagagggcacggggcagggcgcgcagcctgttgcggcgcagggacacc

SEQ ID NO: 107 - DNA, 550 bp, Homo sapiens
  source 1..550: mol_type = other DNA
gccccggtgcaccgcgcgtccagccggcccaactcgagctagaagccccaaccactgcccagtgcctgagttgcagtcttgggtcctttagaaacctggagatgtgcgtaaaattcagatgccggtattcccgaacttccccaggcctcagcatatctcggcggcctgtggacagatgggaggctaccaatcgctccggcgtccgcagcccgacccctgccgccagaccccggacgtcttccggataataaagttcccgctctaattcattttccctaatctggacgcccctaatctacagcttttattgcgcccagttaaaagtcgagggaattcgctgtccctccgcgctcggataattacccctaaatggccacggcagccccttgtgtttcctggagattagaaccccgcagtcatcaatggcagggccgagtgagccgccaatcacctccgctcactccctgagagccgctggcctgggccgcaggaggagaggccataaagcgacaggcgcagaaaatggccaagccccgaccccgcttcaggc

SEQ ID NO: 108 - DNA, 2000 bp, Homo sapiens
  source 1..2000: mol_type = other DNA
agggtgcctctgttcaaattagaaaaaggcgccccctcagggcagactcagcccagctgccaggggacaagtcctggctaacgggagctggagctgggtttcacctccaggtgcctccttggcggggcgccccgtgcaggctacagcctacagctgtcagcgccggtccggagccggagcgcgggaatcactcgctgcctcagcccaagcgggttcactgggtgcctgcggcagctgcgcaggtggagagcgcccagcctgggaggcagtagtacgggtaatagtaggagggctgcagtggcagaagcgagggtggccgcagcacttcgccgggcaggtattgtctctggtcgtcgcgcaccagcacctttacggccaccttcttggcggcgggcgccgaggccagcaggtcggctgccatctgccggcgctttgtcttgtagcgacggttctggaaccagattttcacctgcgtctcggtgagcttcagcgacgcggccaggtctgcgcgctcgggcccggacaggtagcgctggtggttaaagcggcgctccagctcgaagacctgcgcgtgggagaaagcggcccgcgagcgcttcttgcgtggcttgggcgccgccggctcctcctcctcctccgcgacgcctgccggcccgctgccgcccccgccgccggccccgctgcacagcgcggacacgtgtgcacctctggggccaacaccgtcgtcctcggtccttgggctgcggtcgcctgcggaccccggtgggaacagaaacaagagactgtcagcgccacagacgaggtgaggccgggcctcaactgcaggggtcacgggagtggggcggaaatacactttgatcccactcaagcggagcggaggtctgggaggccctgggcccgggagaccagtcttagactcttgccccactgggtatcccatctaggcctcttctggggagggcggcagactcagccgctgtgtcaacgctgtgttgtcgagaccagctccccaccctctctgggccccaggctcccctcagtaacttggggcactcgacccgagcatccgcgaaagccctcccggctctcagcgttgagcattgggattctagactgcatttccgtctctctgcttgggttcacgcgcctctccacacttagttcacacgcacacacgcgcgcgtcctcgcagcacacacttgtctggtgcaggtaagggaaggtggaggcggatcctggggccaaaggtatttagaatctttcaccctcagccgcctgggattgctgtgagagacatggaaacaggctgagccgaggccttagatgagaggatggactggagagtaaagagggagggttgcccctgcatcgagtttttggaccctgatcccacaccagcttctcggtctcgtacccgcccttccgaagaactccagcagaaaggtccagcggtcccctgtgcttgaggcctacagaagcttgtacccaactagggcaggcacccgggtcttccagaccacaggacaggacaggccacggctgaggaggcctctctcctgcctccaggatgaactaaagacccaatccgggatcttcggcctagggctgctctcccagacctggggtctgagaaagccaaaccagccctttccccaaagctctagttctgcagattctcagctctggcccactcggaggtgttcttcaccacctatccacctactgtggggcccggccctgggaccttgaactggcaggtctctggtccagagctaggtcactggctacctgaggtctctgaacccctcacttttccgcttccctgattttggggatttggggacagacacggcagaaagcactggcgacgaactcaaaaactcccgaacgcaaggggcagcggttctcccaacccagtctaatgcacattggcccaggatgtctcaggcctcaccccaggacgtagggctctgaggagctactccggtctctcgcgggct

SEQ ID NO: 109 - DNA, 1000 bp, Homo sapiens
  source 1..1000: mol_type = other DNA
gagaagggatgtggcggggggctcctccggccctggactccctgggtggactagaaaagggcaaagaagtggtcacatctgtgggccagactggtgcgcgatctttggaggcgcagcagcaaggccgcgccagggctgagcccagaccgcccacgaggaggcccgccaggcccggagcagcggcgcgtgcgggggcgtgccgagcgcaggctctagggcccctgcttcgccccagctggaccccgcgggcggtcggtgcagctcgagcgtgtgggctgcgatgccctgcctgagacttcgggctagggatgcgggcgggaagtgggggtgcggcggcagctgcagattagattccttttttttttggccggagggacgtgcaaacttctagtgcccgggccaagagggcgaccccggaggtgcgtaggtggccctccgggttcccgcttctcctagtgcctctgaaaataccgtcagggtaaagggagacaggcagtaagtcttaccaccaccgccctttccccatgtcattggccaaaaactgaacattaagataaagcagctgtttcagtcaatggaaagcggtagggcgaggttgtacccaaaacccggtttagacggccaatgaagtcctaggaaaagccgccccgggggcacgttcaggtggagcggctgcacctcgggtcgttctaagggatgggctgcgtggtacccacggaattcatgggtccaaaaggtcctggtcacctgtccaaacatccatcccctggcgcatggcggttgacaagatggcccggccacccagaggaaggaggatccgggacggggaacttcgcgccgggaagctgtagcccagagctgcagctcagcattcgcaagagattcatcttttttttctctcgtgttcggagaaacagataaacaagacaccgcctcatcagataagaacgtctccttcgatgtcacggatttcaagaggtagctggagaaactgacgtca

SEQ ID NO: 110 - DNA, 1300 bp, Homo sapiens
  source 1..1300: mol_type = other DNA
caggtcaggcagaacttctgcccttcccgctactggcaccccaagcagggatgcactgggatgcgtggcaggggcgggatctcctgggagcgtctcagcccagcagggagtggggaagcaagagggaaggcttaccttcctcggtggctggcaggaggtggtcgctgctagcgagggggatgcaaaggtcgttgtcctgggggaaacggtcgcactcaagcatgtcgggccaggggaagccgaaggcggacatgaccggggcgcagcggtccttcacctgcacgcagagcgagtggcatggctggatggtctcgtctaggtcatcgaggcagacgggggcgaagagcgagcacaggaacttcttggtgtccgggtggcactgcttcatgaccagcgggatccaagcgccggcctgctccagcacctccttcatggtctcgtggcccagcaggttgggcagccgcatgttctggtattcgatgccgtggcacagctgcaggttggcagggatgggcttgcaattgctgcgcttgtaggagaagtcgggctggccaaagaggaagagcccgcgcgccgagcccaggcagcagtgcgaggcgaggaagagcagcagcagcgagccagggccctgcagcatcgtgggcgcgcgaccccgagggggcagagggagcggagccggggaagggcgaggcggccggagttcgagcttgtcccgggcccgctctcttcgctgggtgcgactcggggccccgaaaagctggcagccggcggctggggcgcggagaagcgggacaccgggaggacagcgcgggcgaggcgctgcaagcccgcgcgcagctccggggggctccgacccgggggagcagaatgagccgttgctggggcacagccagagttttcttggccttttttatgcaaatctggagggtggggggagcaagggaggagccaatgaagggtaatccgaggagggctggtcactactttctgggtctggttttgcgttgagaatgcccctcacgcgcttgctggaagggaattctggctgcgccccctcccctagatgccgccgctcgcccgccctaggatttctttaaacaacaaacagagaagcctggccgctgcgcccccacagtgagcgagcagggcgcgggctgcgggagtggggggcacgcagggcaccccgcgagcggcctcgcgaccaggtactggcgggaacgcgcctagccccgcgtgccgccggggcccgggcttgttttgccccagtccgaagtttctgctgggttgccaggcatgagtg

SEQ ID NO: 111 - DNA, 500 bp, Homo sapiens
  source 1..500: mol_type = other DNA
tgcgatcattaaaatcagttccttccctcctgtcctgagggtaggggcgggcagattttattacttctcttttcctgatagcagaactgaggcggggttgtggaggagcgacggaggaccacctctaacttcccttcacttcctggatttgaagcctcagggccaccggcctcagtcctgttacggtggcggactcgcgaggttttccagcagctcattccgggacggcggtgtctagtccagtccagggtaactgggctctctgagagtccgacctccatcggtctgggagcgagtggttcgagttcagatgctgggaaccgtcgcttctccccggccgggctcgctgttttctcctccgctcgccgtcatcaagcccggctatgagcagggctttaaatcctccctccctcacccgcaggtttaccgagcagccccggagctctcagacatgctgcgctgcggcggccagaggaggggtgggggcattgccctctgca

SEQ ID NO: 112 - DNA, 500 bp, Homo sapiens
  source 1..500: mol_type = other DNA
gggcttgggccgcaggcttccctggacttccgcagtcccccttctccccattccagaacctgccgagcccctgctgcatctgggacccgccttcaccgtttcccaatcccagcggttagcccctgcgccccctttttggtctccactttgccgttcgaaaatgcctaggttggtggatcgaccctccgcggagcaaagacggatggctggcaggagcaggttcaggagctgggccaaggtattctctgcttccgcctttgtgtccgcccccccgccccctgctccccgcttcccgccagcatctctccttttctgctcaggagtgtttggcccggcggtccaccccggcttcccgagatacgctagagttgcccccacgtcctgtccgccgcgcccctacccaccgggttgccttcggggcccttcggtgctgtgtagtcggcgtggcgctgtgagctaggcgaacaggaacccccaggcccgccacgtctacgctatta

SEQ ID NO: 113 - DNA, 600 bp, Homo sapiens
  source 1..600: mol_type = other DNA
ttctggggcctggatgggtgcgagcgggacccgggggagtgggagtcgccaggctctgagcaagcaagggctgcacctgcacctctgccgggcatgaagaaaggtaaggaaggaaggagctcacccgggtgggagacagagccggggcgcgcgagcttggtgtgggggcgccactccggggcggaggggaggggctaccagtgacttctccgagtcgggagctagaaagaggcttccggccaggttcccttggaacaggtgtcggagttgttgggagagggggctgcaagaaagaggggtgcagaaactggttcattagatggaggctctgggcggaaccgcgaggacaccctggcagcgcgctgtgcctgcgttaggccgggaggggagaggcctccggacggcgaagtgtccctagggacccagacgcctcgggagcgatccgggccgctgcgaagccctgcccaccaggagtggatccccaggattcacctcccggctgcctgctctgagctgagaaggggatctggttcttcacaataccgtggatggcggggaaggggagggagcctggggtaaaatcccatcttggtttcctcg

SEQ ID NO: 114 - DNA, 1300 bp, Homo sapiens
  source 1..1300: mol_type = other DNA
tgtcacagaaaccccagcagcgcagccaccggactgggttctggaggccgagccgcagtccgtgcggcggcgctgggaagagaaggcgccccggcagctcccctgccaccggccccgaggagcggctggctcccccagcccagcgccgccgccgcccggtaactccaggcgcaactgggcgcaactggggcagctgcgacaccgaatccctcacatctgcaacctgggtgctgcggccactgagaaaatggaggcgcagaccaacgagcggtgccgcgaccgagagacctcggctggcgaaatggtggtgccgggagcctgcgagtgacgccagccggcggggttgtcaaggacaacattcgttttgacgcagccaatggcgccgtcaccaagaaaccatcgactctgagaaaaaagagaggttcggccaccgagaaactccgtacgacaagtgctgtggcagaaaaaccgcctactccgcgccacaggcaaaacagccaatggaaaccccaggtgctgcgaccgtgacaccggcactagagggtctcggatggagaaagcggcgcacggagaccaggaaactatgtgtagcacaactagcagaaaaccgtctggtcggccatccgggagaaagcgcggatcagaaacaagcgacttcgatgcagggaaccgcgcagccactgaagaaagtgacccacgtggcagtggtgccagcgaaacactgcagtttggacggcagctgtggggatgccacagagaaacatgcactgccactgaagtacatccagctccgcggagctagtgttcatatgatcaagaaaccgccagttgggctctgctagaaacttttagtcctcccttaacggctatcctacccacaacagacaatgcctttacccagcacctagcggtgctgagacccgcctgggccagcacagagcgcagagcagtacgggtacggagaaacgccggactcagtgaaaccagccttgcctccagcggattccccggcttcgccggacgccacaggcagagtgccgcggggaaacctctggctccctaaaccgattagattgtgggagtgggggggacactcacaagttgtgtggaagggaaccagcggcaatgggacccggcgagcacttgcccgcagcaaatgcctgcgctgctgcaaaaaaaacaacttttggcgcaaagaatgttgcggccagagagcatccgctgtcgctgacaaaggagtagcaatggcaatgagaaaccgccggcgccacggccgaccgcggcggctcacgcctatgat

SEQ ID NO: 115 - DNA, 2100 bp, Homo sapiens
  source 1..2100: mol_type = other DNA
caaacgctgagagacaaaaagacaccaacacccaccaggactgcgtcctgccagctcttcactccgctgacctgaccttccacgcccctagtcctcgagcggacttgacctgtgggggagtaccgaaccgtccccatgaggccctccaagcggccaggtggcctccgccactctctccacccccacctcctccaccccccagcccatcggtccatcttcgatctgcaaaacacgccgggtcagcgacgcatcggtcccaggcttgtgaccacctctttctctgttacttggggagccaggcccaccgctcaggatcacagtgaggagaaaaaagacacaaacgccaggacagggcggctggggaaggaaactgctagggaccgctcattgtcagcctggcgtgtcccacggatcgcaggacccgtcgaggctttgctctctgcgacccgaatactcctgggcctctcgacctcctcctcggactcaggcgtccgcgtctccggtcatcacgggagaccaattggtttacaaatagtgatgataaacctgggaccgaccttggggctgtgtaaaagtctactgacagatgtaatggagggttgttagcagtcacaaagcctgtcggacccgtagcattagttcaagagactattttcgtgtcgcaccaaaattactgcgcgtgtaaaccaatttccccgacggaagaataaacagagattcgtttgaagcgcgagatgaaaacagatggggtatcgcaaacagttccccaaaatacaacagacttctgggccaattacacgtggttagctctgaatggcagaggaaatagttttctttgctgctaaatgtcacaaaagtcacctaaaggcacagaggaggccgctctgtttttgcgaaacttgctaaaattaatctgcgctgggccacttgcagaaagcagaaccacctcccgcccccacctcgcctccagccgccggggttcaggcgtttgtgaaagacagaacctttgggctagggacccgggcactggtgcttcgaagtccgaatccgccggccgagaaaacgacaagagaaagaaaatccagcgggcgctctctccagcgccaggccggtgtaggagggcgctggggctcggcctgccacccctacccgacattgggaagcagcccctgcgctcccgcggcgcctcagcctccggtccccgccccgaggtgcgcgttcctcctcccgcatgcccgtctcgggccccacggagcaagaagatagacgatgacgaggcgcgcccatccatccgggccgacgaggtcaggcccgcgccacaggcaaaaattgcgcaagcccggccgcagggatttcgcgggcgcctgggtcccaggtgcgcggccgaaatcctcagggaaaatcccgaggggccaacggtctaggccacagggctgctgggcccgggcctggctcagagcgcattcgggcggggaggccgcacgccgcacccgggcctctcctccgagcccgaggcaggcactgagctccgggccagccaggtgcctcccggctggtgcgagaccccgggcctgctgggaggcgtgggcagggcagggcagggctgaaccccagcgactgaatctcgaaggcaggaggcctcggaggtcatcggcccagctcgcctgaaactgtccctgctcgtgccagggcgcgggcagaggagaaaggacagggcggagcaagcccactgcagaactgcggtcggtggctgcgaagggtccgggtcaccgcgctcccggacgccggaagccgcgctggcggggccgcggggagggaggctgggtaccggggccgtccggccggaggaagcggctccggccgcgctgtccgcgcttgggagccgcgtgcagggttcagccgtgtttcagttgccctctgacctgaccccgggcgcacaaaggcctcccgggtgcgccgccatggcccagtcttccagtcgctgccaaattaatgagcccacgtcaggttgggtttacagctcggccgggaagcagccgagtggaaaatgagctcggggccgctccagaggctcccgcacaactgcagaggctgcccgcg

SEQ ID NO: 116 - DNA, 250 bp, Homo sapiens
  source 1..250: mol_type = other DNA
tttccaagacagaaggagggaactaggcgccttttttccactccgctgaccccaacgtctgggctgtgcgttgtaacgcagttggcggggccttcagcttgggatgagggcgaaggggctcgggatgggtgggaaagcaaggaccgggcaacaggtggggaggtggcggacttttgtctcggggaaggaaatcggctgtgctgaaagggcggaaagcagtagcgcacagaactagtgtctgcggggtccc

SEQ ID NO: 117 - DNA, 250 bp, Homo sapiens
  source 1..250: mol_type = other DNA
ccctcctgtggctgcttgggcagacgcctgtggcctgtcggatgcggcccacatcgagagcctgcaggagaagtcgcagtgcgcactggaggagtacgtgaggagccagtaccccaaccagcccagccgttttggcaaactgctgctgcgactgccctcgctgcgcaccgtgtcctcctccgtcatcgagcagctcttcttcgtccgtttggtaggtaaaacccccatcgaaactctcatccgcgatatg

SEQ ID NO: 118 - DNA, 2000 bp, Homo sapiens
  source 1..2000: mol_type = other DNA
tcctcctttgtgtatgtcaacccagaggatggacggatctttgcccagcgtacctttgactatgaattgctgcagatgctgcagattgtggtgggggttcgagactccggctctcccccattgcatgccaacacatctctgcatgtgtttgtcctagacgagaatgataatgccccagctgtgctgcacccacggccagactgggaacactcagccccccagcgtctccctcgctctgctcctcctggctccttggtcaccaaggtgacagccgtggatgctgatgcaggccacaatgcgtggctctcctactcactgttgccacagtccacagccccaggactgttcctcgtgtctacacacactggtgaggtgcgcacagcccgggccttactggaggatgactctgacacccagcaggtggtggtcctggtgagggacaatggtgacccttcactctcctccacagccacagtgctgctggttctggaggatgaggaccctgaggaaatgcccaaatccagtgacttcctcatacaccctcctgagcgttcagaccttaccctttacctcattgtggctctagcgaccgtcagtctcttatccctagtcaccttcacctttctgtcagcgaagtgccttcagggaaacgcagacggggacgggggtggagggcagtgctgcaggcgccaggactcaccctccccggacttctataagcagtccagccccaacctgcaggtgagctcggacggcacgctcaagtacatggaggtgacgctgcggcccacagactcgcagagccactgctacaggacgtgcttttcaccggcctcggacggcagtgacttcacttttctaagacccctcagcgttcagcagcccacagctctggcgctggagcctgacgccatccggtcccgctctaatacgctgcgggagcggagccaggtgaggggctcggcgccgccccgggcgacccctgggggcggcactggagaagccgcccgtcctcataagggattgaacttgcatccactcctctccggccggcttggtcgctggctgcgctccacccgattctcgggatcattggaccgtttgcgcgaaaccagagtggccgattaagggatggggctccgagcaccgggggtggtggcgactgtgggcgaggggaggtgggaccgacccccacccctacactcaaaaaaggccggggcctccttcgagcttccggtgaatttcgggcgatttccgcgggtgtcgggggtcccgggaggaggcagtcacagatccacccctgcagccagcctcctaggcgccggctccggcacgcttcgccggtctgtagatttcctcttcgatttctccccagctcccagcatctgtgacttcactgttaccctccctatccccgcatcacccaaccgcacctgtctgcgggacttaggtgtgcgcgcggggctcatgcgtgtcctccctgctggccacccccacggcccacacaagttgcacgggctcgccacgccccgccaacacgtgcgcggacgcacgcacgcactcctcgcacgtgggcttacgcgaataccagctttcactgccactcgctcgcggccagattcacaggcctgttccggtccactcgcagctcccctctgccgctccctccgccgggctcaggagtactcgtagctgattgtgcgcgcctgagggtcccagatcgcggccgcccaggaccaggcgaggactccggagcctcctctcacctctcccacctgcgccccgggctgggccgggtcgcctggggggcggcctgagcgaggcgcggggccaggagcgctggagcgactgccgctctaagtgccgggcgggcaggactctacgatccttgggccagaggtccggatggtcccgggactccgtctcaagggtcggcgacccctcaacccagaagcctcgagcaggcggacaggcagagctgcccagtggccgaggcgcgg

SEQ ID NO: 119 - DNA, 1100 bp, Homo sapiens
  source 1..1100: mol_type = other DNA
atttgtcgttgtgccattgctgccactgttgttcttgtccagggaaacaccggtggccaacccagatcggatacaatggtgcggctctggactgagcctccaaccacattagccatgggcagcattgttgctgccgctgctgttattttaattatgattgtacgttaaccaccaccttccttcctctgcctcccttcagctgcaatgatgtatgttactttttggtaactggatttcattaacatttatgaactctcataaagtagtagaaaaagcaatttgtgtggaagaattttccacctcattaaacagtgttcttttgggggtcaagctgatattttttttgttgttagattttttttataggtcctttgtccttccctaagccctgggggatgaaaggagagccgtccacccagcgaggggcttgtgtgccctagagggcgctgggccccgcgcgctttcctggctgtccccgccggctttccaccctccccaaagcccaggtgcccaccgtgggtcgctgcggcctttccccttcttggccaaatccgattacttcgcagcctgcagatggcatcgccggctaagggcagcctgcggcaggtccccgagcctgagcactcctcctatctggggcctgagaggacgctctgggctttttcccaggcccagggtgcgcggcctgctagcgcctttcgaggcacagtcccaagataggctcttgtccttcgacgcccccttggcacaagcgcactggcgccctccgctcaacccaccttgcctttggggcgggcttcaaccctgggaagacaggcctgggggaagcgagaggagaggcccgaatagaggttccggctcaatctttcccagacggaggcctggtgtttccagctcagttgcatcttccagccgcgggctcctggcccaaacagaatgtgtttgctttcacaccgggacggcaagcggagtccgcctcagtgagcagcgagctgcgcagtccggacgggtgtcgcccccagagactcgccagccgcccccagacactcgccagccgtccccatctctaatccaccgtccaggcccgggccctgggaaga

SEQ ID NO: 120 - DNA, 800 bp, Homo sapiens
  source 1..800: mol_type = other DNA
ccgtgtctcccttaagaactggggcctcatctccactccagctgcgcgtgcacgtgtgctcccggcaggacgcgcgcccaggagcgcgctgggggctgccccgcccctctctccctcccccgcgggtaaactccgggcatccatcagtctgttaattgcactaattagagatcgcagaggtgttaattggaaaaccctggtattgtgcctgtttgggggaagaaaacgtcaataaaaattaattgatgagttggcagggcgggcggtgcgggttcgcggcgaggcgcagggtgtcatggcaaatgttacggctcagattaagcgattgttaattaaaaagcgacggtaattaatactcgctacgccatatgggcccgtgaaaaggcacaaaaggtttctccgcatgtggggttccccttctcttttctccttccacaaaagcaccccagcccgtgggtcccccctttggccccaaggtaggtggaactcgtcacttccggccagggaggggatggggcggtctccggcgagttccaagggcgtccctcgttgcgcactcgcccgcccaggttctttgaagagccaggagcctccggggaagtgggagcccccagcggcccgcagactgcctcagagcggaagaggcagccgcggctttgacccagcttccttccgacggcatctgcaggagcctctaggcctgacataggctccgaggtgccctggctcccccacggggaatgctgagggttgggccactaggtcctgcctaagtgcaggacctgagcctcagacaaatc

SEQ ID NO: 121 - DNA, 300 bp, Homo sapiens
  source 1..300: mol_type = other DNA
gggattgccggctttgagaaaatatgaagaaaccgatttctccttccactttgccagtgcactttccttccactttcactggtgctgggggcggcgcactctttacgacatataagcggaaaattctgcaaaagtggcccccggggatccccgcccgacccctgtctgtcgctaatgtgggcctgtctccggaaattcgaggttgggcctttgcctgaatctgttgctattgctccccttgctaccgctgacacttggcaccgccgcctcctagcagcggccagacgcggggctgggggc

SEQ ID NO: 122 - DNA, 400 bp, Homo sapiens
  source 1..400: mol_type = other DNA
gttgcgagcgcggcacaggttgctggtagcttctggactctggaggcttggccttccttctaagccgatggcggggaaagaacctcgtttccacagcttccccgacccccgccgcttgccatttggggacgggaagcgcgcccgggtcgcttcacgtccctctgggccggagccctttccatggctggctcctctgggggcccttgggcctgtgagcagcgtctacttccctcagagaagaatcctttccttcccccatcgaagtgtccctttctgtatcctgaaataacccctcctgggtgaggccagttcccctctgtcgccctcctcccgcaggcgtccgggagcctcgtgaggaccccgtgcagttgagtccaggcgacaggtgcctccccaggtg

SEQ ID NO: 123 - DNA, 800 bp, Homo sapiens
  source 1..800: mol_type = other DNA
cagtgcgccccttaccggagcacccatggcctcccgcgttaccccaaattttgtaggcagactgtcagagttcgaagccagctgtgtcctctgcgggccgtgtgaccctaggctatctgggctgctcggagccttagtttccctagttgtgaagagggagggtgtgaccatggcccggagctctccgaaaggctgtgcggattgctcggtggcgggatgtggagcgcgtcttctatgatgccaggtgctggccaagcgctcgatgcaggctgctccagttaggtcgatgcgatggcgggaagcactttcctctgcaatggagagacgccgacaccccgagcccgaaggcttgcaaggcgcgctctcgccactggggtcggggatccgtgggttctctatcccgcttacccactccatccttagcagctgtcgtcggtcccagacctctaccttggagagaccaaggcggcccagagcccaggagactactgcgcggtacgccaggatccagaagtggattctgacttctaaagacccctcccaagccaacgctatcagggtccctgcaagcggttgactgtggcggaggcagaaccaaaacctttgctctgcccgcggcgctccagcctctcacccaggacagtgctctgggctccagccgctgcagtggggtcgggacacagacgccgagttagaagccccgccgctgcaggtccctgcttggtcggcgcggtgacggtgtcgctggcggcggcgggggccttcctttggctgcccggccatttaatcagagctattat

SEQ ID NO: 124 - DNA, 1000 bp, Homo sapiens
  source 1..1000: mol_type = other DNA
tttagtatttaaggagaaaagcctcattttccagaatcgaataagcgaattaatcgcacaattgtgtagaatggaactcagtctgtaaaaaatcaagaccaacgtactttttaatattctaacatctccaagtagtagttacaagtattgtacccatgaagtccaggtaattaatttgttcaatgtcacactgttaaaagtcaggtgggctccaaagcacagtcctaaccagcatgctctactgcctcctctgaggcaacagccgaagtgcagaccactgggaataaatagctgcccggtcttccccactcctaaattctcccgacagaccccaaagcctctctgagagcctctctgaccgccctgcggcccaccccgagttcccggcatcctctgggatccctcttcctggagccaaaacctacgcaggctcctttcctccgagctggttgctaggtgatctccgaaggctgtccgaagtctcgcgagggcggacccgttgcctgatgacgagagttgggagtgtggctggggctgcggatctccagcagtggcgttacttctagcggctggataccgggttctccgcgagatcgcgagatcccgagatattctccccgcacggaagcgacgactggcctggccagaggactcgcgtgggagcgaggtgccggccccgacaggacggtgaggtatgcagaagtaaggcggggcgccccctgcgggaagcgagcgcgccccggaaaatgagcgcctccccacaccaaggtgtccaggagtgagtgcgggaaggaactcggccgcccggagttgtggcctcatcgtgcttcccgccaaaaacgccttggtactgtcgggacgcggctaagcgtggacgcgcccgcatctgcccctcctccgcagtggtggaagacacccgcggagcgccggtggataagggccgtttcctgagaccagagctgtatccgcagcaggtcagcacttcgtgcgccctgtgtgc

SEQ ID NO: 125 - DNA, 300 bp, Homo sapiens
  source 1..300: mol_type = other DNA
agcggcgctgttcccgggctgggtgcagctgctaaggacaaggcccctgctccgaagaacgcggtggctcggggataccctgaaagggacggccatggcgcacatgggatgccctagggttcgtgggagggcatgcaggcgcagcccccgcaggggttggcctgccagagaaggcaggggagagcactcggggctgcacaaatggtgtggccggagggaaggtgcagccttgtgtgtgtctggatgagggctgggcataggagcttggtatttgatcctgaaagctctgcgtttccaaag

SEQ ID NO: 126 - DNA, 600 bp, Homo sapiens
  source 1..600: mol_type = other DNA
gagtcatacttgtagtcacatccttttcctttctccaacccactggttaatcatgaaaggctcttctgattggctgcctcctggcagtagtgcctcagcgcgacggttcgggagcaaataaataattcccgctgggaagctgtttctcagacaggagcagcgacacccctgccacgcctgccgcctggagttgagtggggtaagcacgccggcctccaggaatcgacggtgccacgtggttcttcttgcacttctcttcttctccagtttcaggggacaccgtggggtgtgcgagcccgggggagcgcagggaagggcgggttgggctgcaggtgggaatgtgcggtccttctgcgccctcaacagagcttccttcctttttgccaaggtccccgtgccgccttcagcgcgcctccttatgcacctctacctctgctgcagcgtacctcttccgcagccctagcggcctccccgaggggcgccgcggcctcggctgtccctcccctgcctggcacgaccacctgacccccagcgacccaagaagcaagttgtgtttgcagacgcaaaggggctgtcgttggtatcggtgcactggtttga

SEQ ID NO: 127 - DNA, 1700 bp, Homo sapiens
  source 1..1700: mol_type = other DNA
acactttctgtgtgggagggcacaagacatgggctatgacatggccagagaccccaccttctttacacatgtaaaaaccaaccaaatcaagatgcgtcaacggtgattcttcctcccacattgtttccctttttaaactgttattttttcaatccatggagcagttgagaaacgggtatgcatctctcctcccctccccttctatcaaagcctgtaagacacataaggaaatccaaagccacagtaatagagagagagagagagagagagagagagagagagagagagagagagaaaacagaacaaaagaaatcctccttggcttgtttttccagggtggccaggcaaggtgtgaaaatccatatttccctctgggctggcaggtagaagttactgggaaggctgcgctcccttctctcccaccggctctcacatccaggctgttccctcaccctcagcctcccccagcgccagcttcctcctccgcctctctgcagccaggcctcccctgcaaggcggaccttggcccaccttggttccgggccaaggcggcgggaaaggcaccgctacctgcagccgcacgactccaccaccatgtcctcgtactgcttgtagaccacattattgcccgcgtcgatgtatagaatgctgatgggagtcaatttggtgggcacgcagcagctgggcggggtggagccggggtccatggagttcatcagcgtctggatgatggcgtggttggtgggctccaggtgcgagcgcagcgggaagtcgcatacaccctcgcagtgataggcctcgtactccaggggcgcgataatccagtcgtcccagcccagctccttgaagttcacgtgcaggggcttcttgctgcagcgtagcctggacttcttgccgtgccgcttgccatggcgactggcgaaggccgtgcgccgccgccggcggccgggcgagggcagccaaggcctggcatccggggcgcccgacggcggcggccacgacccctcggcgcccgcgcccgggcccgcagcctcggccgagcccagctgctcgcgcatctctgcgaacaggttcttgcgctgggatctggtgaataccaccagcagggcccgctcctggggaggccgcaccctccggccgaagcccagactccgcaggtccgggggcggcggttgctggggtccccgcgcgcgcgcctcggcctccccggcgtccagctcgccccatgcggcccgcagctccaagcacagctgcttccagggctggtggcgcaggccctgccacacgtcgaagacttcccagccggccggcggcgccccctgcgggtccagggtccgcgcgtccagcagtaggggcgaaaggcaagggaagagctgcacgtggagcggcccggctggtggcccccagggcgctgagggcgcctggcgaaagagccgcagctccgcgcccaccagctcttctttgtctgagagcatggacacatcaaacaaatacttctgtctccggagaggagtgtgcgagagatcgtctgcgagataaaaaataattacagtcagtttcacttaagggggagatcagcccggtgctcttcggccgccccgggaggaaaagggcggggagtgggggcaggtcggccgggcagtccagcttgcccggcccagggcctgaccaccccggctccccatctggctggtgcatgg

SEQ ID NO: 128 - DNA, 2000 bp, Homo sapiens
  source 1..2000: mol_type = other DNA
gcccgctgtgaatgtaggtgaggtgatcccgggaacctgggtctgaaatcagacctgtgttgccattgggagcacggagagaggggaagcgccctgcttaggcccaggccgggcgtcctggtggtgggaccgcagccgcactcacctccaggccaacggacaaggttcctgcaagccagcagggccactctgtgcttggcctactgcagctcccctgcagctcctttcctctccctccccggagcgctctcctctctcctctcccctctcttctctctcctctctcgtctcctggggcatcccgggtggagggatgtaggggtcgctcctcggtgccaggccgggaagcagctcaggcctcccaagagcttggcgctcagtctgggaaaaggggttcctctggcctcagggacgttctccgcccccaccccaccccctgggagcctgaaccatctggaagggatcttagtcgggggttgggaggagagcccgtggataggaggagggggcgattctaggccgaatccagcccctgaggtgtcacttttctttcctgcggcccgtcaccgctgatagatggggctgagggcagaggaaggaaaaagaaaacctccgaggtcagtgcggggcgaggtgagcccctcccagggccctctggcccaggaggatgaagcgcgccggcttcgctcttgcacgccggcttgccatccgggtaagcgcgggaaaggcggccacagggcgcggcggcagcgcagcgcgtgggatctcacgacccatccgttaacccaccgttcccaggagctccgaggcgcagcggcgacagaggttcgccccggcctgctagcattggcattgcggttgactgagcttcgcctaacaggcttggggagggtgggctgggctgggctgggctgggctgggtgctgcccggctgtccgcctttcgttttcctgggaccgaggagtcttccgctccgtatctgcctagagtctgaatccgactttctttcctttgggcacgcgctcgccagtggagcacttcttgttctggccccgggctgatctgcacgcggacttgagcaggtgccaaggtgccacgcagtcccctcacggctttcggggggtcttggagtcgggtggggagggagacttaggtgtggtaacctgcgcaggtgccaaagggcagaaggagcagccttggattatagtcacggtctctccctctcttccctgccatttttagggctttctctacgtgctgttgtctcactgggtttttgtcggagccccacgccctccggcctctgattcctggaagaaagggttggtcccctcagcacccccagcatcccggaaaatggggagcaaggctctgccagcgcccatcccgctccacccgtcgctgcagctcaccaattactccttcctgcaggccgtgaacaccttcccggccacggtggaccacctgcagggcctgtacggtctcagcgcggtacagaccatgcacatgaaccactggacgctggggtatcccaatgtgcacgagatcacccgctccaccatcacggagatggcggcggcgcagggcctcgtggacgcgcgcttccccttcccggccctgccttttaccacccacctattccaccccaagcagggggccattgcccacgtcctcccagccctgcacaaggaccggccccgttttgactttgccaatttggcggtggctgccacgcaagaggatccgcctaagatgggagacctgagcaagctgagcccaggactgggtagccccatctcgggcctcagtaaattgactccggacagaaagccctctcgaggaaggttgccctccaaaacgaaaaaagagtttatctgcaagttttgcggcagacactttaccaaatcctacaatttgctcatccatgagaggacccacacggacgagaggccgtacacgtgtgacatctgccacaaggccttccggaggcaagatcacct

SEQ ID NO: 129 - DNA, 2000 bp, Homo sapiens
  source 1..2000: mol_type = other DNA
cactcccccgccgcctccgcccctaaccctcggccccgtgcgcgagcgagcgagggagcgaacgcagcgcaacaaaacaaactagtgccggcttcctgttgtgcaactcgctcctgagtgagtcgggggccgaaagggtgctgcggctgggaagcccgggcgccggggacctgcgcgcgctgcccggcctggccggagcctgtagcccgggggcgccacggccgggctcgcagtccccccacgccggccccccggtccccgccgagccagtgtcctcaccctgtggtttcctttcgcttctcgcctcccaaacacctccagcaagtcggagggcgcgaacgcggagccagaaacccttccccaaagtttctcccgccaggtacctaattgaatcatccataggatgacaaatcagccagggccaagatttccagacacttgagtgacttcccggtccccgaggtgacttgtcagctccagtgagtaacttggaactgtcgctcggggcaaggtgtgtgtctaggagagagccggcggctcactcacgctttccagagagcgacccgggccgacttcaaaatacacacagggtcatttatagggactggagccgcgcgcaggacaacgtctccgagactgagacattttccaaacagtgctgacattttgtcgggccccataaaaaatgtaaacgcgaggtgacgaacccggcggggagggttcgtgtctggctgtgtctgcgtcctggcggcgtgggaggttatagttccagacctggcggctgcggatcgccgggccggtacccgcgaggagtgtaggtaccctcagcccgaccacctcccgcaatcatggggacaccggcttggatgagacacaggcgtggaaaacagccttcgtgaaactccacaaacacgtggaacttgaaaagacaactacagccccgcgtgtgcgcgagagacctcacgtcaccccatcagttcccacttcgccaaagtttcccttcagtggggactccagagtggtgcgccccatgcccgtgcgtcctgtaacgtgccctgattgtgtacccctctgcccgctctacttgaaatgaaaacacaaaaactgttccgaattagcgcaactttaaagccccgttatctgtcttctacactgggcgctcttaggccactgacagaaacatggtttgaaccctaattgttgctatcagtctcagtcagcgcaggtctctcagtgacctgtgacgccgggagttgaggtgcgcgtatccttaaacccgcgcgaacgccaccggctcagcgtagaaaactatttgtaatccctagtttgcgtctctgagctttaactcccccacactctcaagcgcccggtttctcctcgtctctcgcctgcgagcaaagttcctatggcatccacttaccaggtaaccgggatttccacaacaaagcccggcgtgcgggtcccttcccccggccggccagcgcgagtgacagcgggcggccggcgctggcgaggagtaacttggggctccagcccttcagagcgctccgcgggctgtgcctccttcggaaatgaaaacccccatccaaacggggggacggagcgcggaaacccggcccaagtgccgtgtgtgcgcgcgcgtctgcgagggcagcggcggcagggggaggaggaggcagaggcggggtggctggaccctcggcatcagctcattctcccctgctacacacatacacacacaaataatgtttctaaaaagttcagttgcgactttgtgcctcgcctgtcctgttcatcctcgtcctgggccggggaatgcttctgggggccgaccccgggatgctggctaattgctgccggcgggttccgtcgccggtgtgaccctggacggcgcggacggcgtacagggggtcccgggaggggcagtggccgcggcactcgccgccggtgcccgtgcgcgccgcgctctgggctgcccgggcggcgcagtgtggacgcgg

SEQ ID NO: 130 - DNA, 800 bp, Homo sapiens
  source 1..800: mol_type = other DNA
ctgaaaagccgtcagggaaaccacacatgttcaacccctggcggctcccccaaacctctcatttccagtaactgtgtgtttccgctcgtcaacagctgaaaccgagcggaacttggggggccccaccacgcggccctgctgtgcggcacggggctcatctgtcccccggctgcggggagtcagctctcaccgcccacctccttcccagatagtctctgtgcccactcgacggcccggcaagcccagcccctgcctgccacggccacagcagcctcagagagctgccctctctggccagggtcagggcctgagctgctgcctcccgcagggtcgagggcaggacacttgtctgaggcttgggtggggcaatggcacctcctcagggcctcagcccccgggcaggctcggtgaccatgggcctacagcagggaaaattctgggccaaaagctccagcctcctactagggcatctgtctgcaaatgcaccttaacctgaccgcttgggctgtgggggagcctgtttcagggaaagtgagggacgcgccagtttcctcctttggacttgatgaggcacgaacgcatctctaataaagccaggtctccccgccgtggctccctgggcgggtgcctgtggctcgggccatgagtcacgctgggtaaccccactacggggaagagggcaggaagctgggagccaccgcctctgtgcccggttgtcatctcggcacgagggcgaccgtcggcttcgtcctgccctcatggctgagggcttttgggatgtggcgggagacgggggagtc

SEQ ID NO: 131 - DNA, 500 bp, Homo sapiens
  source 1..500: mol_type = other DNA
aaatcatcagaatggctaaaatgaaaaagacagacaacagcaagtgctgacaagggtgtggggcggccaaatgctcctgcactgctggcaggggacctgagaactgcagggcattccctggcttcctgcccctcctgggactggggaccccccagggacagcctaagggaactgcatttatcttcacgtctgccaaaagataacacgaagatgttcaaagctaagcccccaggctggtaagagctccaaggcaccagcagtgtgtgcagaactggggggagtctgttctcccagggatgctcccatcacctgctgccagcagtggggcatgccggtcccctggggtgtggccaaggggctgtgtctcctgcccgggctgccggcccctctcaggttcactttcccatctctaagcccacgtctcgctgcagttcaagtttgccaggccaccaacgggtgacacgcccggcgcagtgggggactccgcactttctgcgcac

SEQ ID NO: 132 - DNA, 1200 bp, Homo sapiens
  source 1..1200: mol_type = other DNA
accctttgtgcctgggtcccataaacaatgtgctttttaaaggggagccccctcccagctccggcctttttctccagcgtgggcagccaatcagctgcgcagagctgcatagctggaccgctttccattctgagtagcaacaacgtactaatttgatgcacacatggatgcctcgcgcactctgcaaattcatcacccgcatcttgcattagtcatctgacggactgccaagtgtttcattttctttccatgtgactttattattaccacctctctcctctcttccaaaaacctcccaaaaagggcggtggggcggggggcggggcagggagagggagagaaatccagcagacatctagctctgcctttctttcccagccacagccagggtagggctgataaggcgctgatgcgttgatggcagccttgcagagctagacctgcacttaacttgcagctgcctcccgagcctccaagatgtccacgccctgggtgacaggcggcagggcgctgccccgtgctcccccggctctgctcgacagcagcacgcagtgagagcctcgccgccgccgaggagcaactcatggtgcctccgctttgttttagttcatcaaatttctacgactcattaggcactttgccactgctcttcttcctcctccttccgcctccccgctcccccacccccactattttttcttcctgtccctcatcgtgccgccctaactctggctcccggttccgtttttgacagtaacggcacagccaacaagatgaacggagctttggatcactcagaccaaccagacccagatgccattaagatgtttgtcggacagatcccccggtcatggtcggaaaaggagctgaaagaactttttgagccttacggagccgtctaccagatcaacgtcctccgggaccggagtcagaaccctccgcagagtaaaggtacagagcgcggggcgggggtcgccaggcgtccaggtgggcgtcgcggggcactggggctgtccgagcccccagcctgcaggaggaagggcgggtaggcaggagggctggaagcagccggtgctggcggcccctgtgctccaggggctgctcccgactcctccccgcacccccgcccgcctgcccgccgggacaggttggaggcgggagagagggaccgaggcagggcgggagcgcagaggctcggtc

SEQ ID NO: 133 - DNA, 800 bp, Homo sapiens
  source 1..800: mol_type = other DNA
taacaaataagccgcccgtggtccgcgctgtgggtgacccttggcgccttcgaggtctggagccctagggtaaataaggaaacggggcgcctctagagttttaaatgaactctgttattggaagcttcagtagggaccctgaaaacaattaacgtcttaattagcattttaatgtctccattattacggcgcgggctctagctcagccctttaccttaccttctcaccgttaacaggggagggggattgtatttttagttcatctttttatgtttttgagttgttatcctgtctgtctgattccagcctcgagggtttgatgatgcggcccgagcctggctgtggtcgcctgtcggggctggagcgggaccctcagccgggccgggcctgggggctaacgttttcacagtgcgccctgagtttccttgggttactgctgggaccgcgcaggaggaagcaaagagtttttcgagctagaccaacaggaaacacattgacggaaatgttgccatagcccatggggtggctttaactggccgcccccgcgggctgggtgtgaaatcagaggaggccgcggctcccccggccaggattggaggctcctcgcgcaacctaatgcgggtgtccgggcccgagcgcttcccgcgcagccaggccttgtcggtgcagcagccccgctcctccccaacacgcacacacccggtgttcgcaagtgcggctcaccaagggagatccaagggggcaaaaagttatgtataaatccgagagccactggggaaagagggtcgtggtattgtaag

SEQ ID NO: 134 - DNA, 500 bp, Homo sapiens
  source 1..500: mol_type = other DNA
ctaccctgtgctatcctgagctgtagtcttctgaaatgatcgtttggcttcccagccaaggcagggctcccccaaagttcattcccactcttgcagtttcacctcgggatgcttccgcagaatttcagcgcctaagcagacaaggtcaaagtaaaccgcttcaccgctgcttctggcgcaggggcccagagcgcgtgcagctccccagcacagaccaacagcaggagaggggtccgggcgggagccctgggctgtagataagcaaaacgcacccattttctctcctatttactccagaggcacctctcctcccccactcctggcatctctttatcactggctccctctccctgtggcatatttttgggtagtagaatgctgaggtcacagggagcggctctttatccaagcagtggggacatcagcctggagccctgagcatgaaccagcaagatgcagactctcgctcttgactttgggctccaggagctgccccgacc

SEQ ID NO: 135 - DNA, 1100 bp, Homo sapiens
  source 1..1100: mol_type = other DNA
cagtgctccgctccgggaaattgcatcgtcacgacaaacgggaccgtgataaaacgaccctttccgtccttatttgtagatcactcagacgagattgaactgcacttgtttccccttcgaggggagccgcgttttcagggtagccgaaggcttggggctgagggggggccctcaccaaggcgcgggtgggggccggagcctcaactcgatgagaagtgacaggcgtttgggggatctgggctccggccgggaccagcgcaagcagggactttgcggggacaccgcttctccaacagagcaaggcctggcccacgtttccggtttctcctaacttccttttattgccttcctttgcttcgcaagttccatctacccctccagctacagagccccacctctaggcacaggaagcttcccggaaaaagaaaggctgtcccagaaagagaccgagagagactttccaaacttcgggcatagccacggcaattcccagtctgctaatgccaaggcgggcgcgtaaggccgcctaaatctagacctccctcctcactcatttcaaaaaataacaacgtgccagccacctccgcagataccgccggctggtgcttgcccaggagacgccagggccagagcgccactcccagcatcgaaatggcagagagaaagcgcagctccaaattccccttcagaggttaagcctcaatcattgtgtcccttccctagggactgctggcgctctcgcccactggcgatgattatgcgcctagaactcgaccgcgaagcaactaataggaaaacatatggtgtcaatttggatgctccgcgcctcgcgcacacccgggaacgagcggcacaaagccctgccggccggcccgcgaccccgcgcccctcggggcctgccagccgggccgcagcgacaaacgctcagggctgcgcgccctggctggggcccgcccgagagacagcctgcggctggggagtctgagctccaaggggagagcccagccgccgaaggcgagcctaccggccaagccctggggtccggcaggttctgcacaactactcccgcaaagctcgccacctttgtgccctttcctcag

SEQ ID NO: 136 - DNA, 500 bp, Homo sapiens
  source 1..500: mol_type = other DNA
gggccctcgcggctcaagcgccagcgctggagagagagtctgagggtaccacgggcgtgctggcctgggtgctcactcccgccctccttcatgagcggctttcctctgggtgtgtccagggcatcacagagctcttctgcccaaacccggaggcctaccagggcctgcccaccttgcctccttccacactctctgtagcagcagccgcagccatggcggggatgaagacagcctccggggactacatcgactcgtcatgggagctgcgggtgtttgtgggagaggaggacccagaggccgagtcggtcaccctgcgggtcactggggagtcgcacatcggcggggtgctcctgaagattgtggagcagatcagtgagtgtccgctgcccgcttgctgaactcggcaccatgggcggccgccacgggtgtctctgggcacttccgggccatccctgctgctcagctcccgataatggtgtcacggtgactcaggcattagc

SEQ ID NO: 137 - DNA, 600 bp, Homo sapiens
  source 1..600: mol_type = other DNA
tgtttacggaatcgggatcgaggggccgataagtagtttacacgccggccagagcagagggctggaggtcggagttgggggctggaggaacgggtggcgtttttaggattcagtaacaggatcacagctttttcttgtggtggaagctattggaatttggggagggtagcacgaggggtcctgcagctccgcgtgtgaaaaagcgtttaggtaggcgatgaaagtagttgatctgagccatggcaggcgagccccgaatttttgctgcttccccctgaaagtgtttctttaggaggagaggacttgggccacacaggacccggtcctaagagagcgattccgggaagcggacagatcgaagagaccttctgggcgaagcggcagggcagcctcgcggggctgggagtggatctgaggtcccgacccaggcggctcggagtgctccaggagccacctgggtctgcgggcgcagcgcggcggggcgggagcggtggcccgcaggggccgcggcctgcgatgaaggccggggggcagcgctagcagcgaggtgccacagtgggccgaggagtctgggctgtggcccagggtaggaccggctca

SEQ ID NO: 138 - DNA, 150 bp, Homo sapiens
  source 1..150: mol_type = other DNA
acctaaaccaagctctccctccctgccgtctccttccctggcctgggtctgaaggagaggaggtgcccagaagttcagagcggcataaccacagagatactacctaattaacataccagaagcataaagaactcatttgcattggagagt

SEQ ID NO: 139 - DNA, 900 bp, Homo sapiens
  source 1..900: mol_type = other DNA
ataactacgggggtgggggtggggaaggaagagatccaaggaggcagaaggctgcggtcaaaatattttggggtggcagagtcacgtaggatgtggctgtgggttctggcagcccagagattcagctcccgcctcctccctcagagcgagtccatagctaccctcacgtcccccgtggcggtcctcgccacgctccggagcgggttacccatgagggtgctagacctgggcagcgggaacctcgaagaggtggagattgcaggctgggactccagatttcgggcagggatgcggggaagggaagacgcctcgctggaggcggaatggagggcaaggcgaaggaggatggtgcaggaaacggcgacaaggcgcccggccaggcccgcgagctaccgagacccgggttccaatcctccccccttccgcaaacgcccgggttcgaggtacctggcgggcaagggccgcagcggagcgaagcgggctggccatggggaggctgcggggacgcggggctgcagagagcggcagtggcacggagcgcgcggctggaagcgaaagcaggcggtgtggccaagccccggcgcacggcccatagggcgctgggtaccacgacctggggccgcgcgccagggccaggcgcagggtacgacgcaacccctccagcatcccttggggaggagcctccaaccgtctcgtcccagtctgtctgcagtcgctaaaaccgaagcggttgtccctgtcaccggggtcgcttgcggaggcccgagaatgcgcgccacgaacgagcgcctttccaagcgcagatatttcgcgagcatccttgtttattaaacaacctctaggtgaatggccgggaagcgcccctcggtcaaggctaaggaaacctcggagaaactacat

SEQ ID NO: 140 - DNA, 600 bp, Homo sapiens
  source 1..600: mol_type = other DNA
cagtccagccgcttgcctcacttcttcccgcttgccttatctccccgcagacgtggttcccctgcagcccgaggtgagcagctaccggcgcgggcgcaagaaacgcgtgccctacactaaggtgcagctgaaggagctagagaaggaatacgcggctagcaagttcatcaccaaagagaagcgccggcgcatctccgccaccacgaacctctctgagcgccaggtaaccatctggttccagaaccggcgggtcaaagagaagaaggtggtcagcaaatcgaaagcgcctcatctccactccacctgaccacccacccgctgcttgccccatctatttatgtctccgctttgtaccataaccgaacccacggaaagacgctgcgcgggtgcagaagagtatttaatgttaaggaaagagaagaaccgcgccgcccggaggcagagaggctccatggccgtgctgctgggccatccccaactccctatcccatccccagcctccacccccatccagatgggactcacgtggcttcaacagctttggaaatgggtcccgagtgggccgtgcgaggaaggctgtcgacctctactcctccttgc

SEQ ID NO: 141 - DNA, 1500 bp, Homo sapiens
  source 1..1500: mol_type = other DNA
caagatcgactttcttaggaagggggagaggagggaactcttcacgaagggaggtgggagtccacctcagacctctattggaaggaaatcgagttgttccgggggactgaggtctcttgcataaggcatgggatccttattattattattattatttttaaatcccccgcggaggagctctgggcaaatgaataccgaggcgccgctctagctggttaggcttgggatgcgataactcagtgccctcttgcagacttgcatagaaataattactgggttgtcgtggaggggacacgagacagagggagttctccgtaatgtgccttgcggagagaaaggtccaagaatgcaattcgtcccagagtggcccggcaggggcggggtgcgagtgggtggtggagtaggggtgggagtggagagaggtggtttctgtagagaataattattgtaccagggcccgccgaggcacgaggcactctattttgttttgtaatcacgacgactattatttttagtctgatcaatgggcacaatttctaagcagcgcagtggtggatgctcgcaaacttttgcgcaccgctggaaacccactaggttgagttgcaaaacgtaccgcgtagacgcccctggtggcgccgagagaagagctaggcctgcccagcacagagccggagagcgtcgggccttccggaagggtaagttctccgccaaggggtcccgagggagctggacgtctgaatctggacttgcccccagcttcggggttcgattctgggttttgcgcgtccccaacccccagggctttccgaagcatggcctggctccaggcccggtcctgtaaggactggaacggcagcaaaatgtgcagggaggcagtcggccggcagagctgcggcgggagccaaggtcaggcccgcggggagagcgggcagcttccagcgccggccacaagctcccaggccagctgggccgcagacccctttgcttccagagagcacaacccgcgtcctttctctcagccaggctgcagtggctgccccgagcttcgctttcgtttcccaagctgttaataacgatatgtccccaaatccgaggctcgtgtttgctcccagatgccaagaacgcaacccgaaatccttctcccaaaccctaggtcgacgagatgagttcctacttgacctctgagccgaggtgggccggaaaccgaggcctaggccccgccggggctgcaaggaaaaggggaaactccgagcgtagcgtcttttccttgtggttcctttctccggcatcccggactgcgggccctgcagccacctggaccggcattcaaaggattctgcaagtccagcttcacagactggctttcccagacgctccgaagcccgcaccacgaacagaataaaggagagacgagagatcgcaactagatttgagaatcctcgttcttttccccaatcgttcgggcagtaaactccggagccggctacagcgcgcatcctc

SEQ ID NO: 142 - DNA, 500 bp, Homo sapiens
  source 1..500: mol_type = other DNA
actgtcctcctccctcaattgcctattttttgcccatagctctaacttaaccctgtgatcaccccagatcgctacttctgacccccatctcctctcccacaccaacctccagcgcgcgaagcagagaacgagaggaaagtttgcggggttcgaatcgaaaatgtcgacatcttgctaatggtctgcaaacttccgccaattatgactgacctcccagactcggccccaggaggctcgtattaggcagggaggccgccgtaattctgggatcaaaagcgggaaggtgcgaactcctctttgtctctgcgtgcccggcgcgcccccctcccggtgggtgataaacccactctggcgccggccatgcgctgggtgattaatttgcgaacaaacaaaagcggcctggtggccactgcattcgggttaaacattggccagcgtgttccgaaggcttgtgctgggcctggcctccaggagaacccacgaggccagcgctccccgga

SEQ ID NO: 143 - DNA, 900 bp, Homo sapiens
  source 1..900: mol_type = other DNA
ctcagggaatcacatgtccgcctggcctggcctggtaccaaatgtttatagacaggacgagggtcgctggaatcgcctcgctcctttcagcttggcgctaaggcgcgaatctcgatcctcctagtatttctctggcgtctgtctctatctcagtctctgcttttgtctctttctccctccctccgccccagtctttccgtctctttttcctcgaatgcacgtggaattcggaattgaaaattgaggtcagaatctccctttttcttccagttatccgcgccgctgccccacgcctagcggcttggatctgcatagacatctatctacccgcaacaagatccgagctgcagaagcaaacctaatctgtctccgcaccatcccctgctctgtagacccactgccccatcccacgccacatccttgaggttcaagtagcgactccagcggatgattcggagaatgccctgctttccaaaggccccaacccgtgtttttattttctttttcctttgcccgcttgaccaactttggtttctttcagggcccggaggtgcctgcgccgcgcttggctttgctttccgccgccccaggagacccgggactgtggtttccgctcgccacatcccagcctggtgcgcacacaagagcctggcgagcttccctcgcgcgcttacagtcaactactttgggcctcggtttccctgctccttgtagatcagagaagggacgggcgaaatgcctgcgagggagggttggcgaatgggttggttggtggcaagactgcagttcttgtacatggacgggggttggggggtcaacactggaagaactcctgcctgacgccaagagccacccgctttccagctcgtcccactccgcggatgtttacccaccttcatg

SEQ ID NO: 144 - DNA, 500 bp, Homo sapiens
  source 1..500: mol_type = other DNA
tttggggcacccaacccttcccaagcctcggttttcccgatcttgtgggatccttgcggcgcgaatggggttggaagcaccttggaagctacagagtaccgggtcgggacaatttccggcactgccccagttcagtggtttatagaaaatttctttctctctctcaggtccactaagaccgagagagagagagaagtcgactctggcacacccgggcgaggggctgccgggattcgggagctggcgcggttgattttttccgagaatcctccacttggggtgacgtcgggcagcgcgcgcgggccgtgaggttaatgcccaggcttttctctaaagcgtccgggaatgatccggcgaataaaacgggtgtctgcaaagttaatgaattgtacaaggaggctgagggtggggacttcgacccggggagccagaggcggttctggtggacgcttccccgtgcgcctaggggtgcgctgggctttcccagccgaggtctgcag

SEQ ID NO: 145 - DNA, 2000 bp, Homo sapiens
  source 1..2000: mol_type = other DNA
ccagacagttaaggtaaaacgttgaagtcaagaggaagtagtgagtctgttgccaactggatagggttggtcctgtcccatctaaatgtattagaattaagtggcttttaaaaatgagctggtcatcttcagcccacgggctggccaatttggaacttaatgggcctttgcgtcctccttccctgagcctccttttattccagacttctcagtgtgagtctgtgcgtccctccgacgatctcagggagtggggtgccttcatctgcctgttccctgttcctcaggctgacgctcccgctgtcctccccgcctcccctcactccttttctccctcccttcctccttgtggggaggctcttggccagggtccctgagcccgggcgggtgctggcagaggacgcagaaggggtgaggtcacgtctcccttgagccccgagccgctggcttttcagagcctcgccacaagccggcggccagagccccagaccacacagaccgtgcgctcctccgccctcccggcgccgccggcctcgcccatgtctcagtacgcccctagcccggacttcaagagggctttggacagcagtcccgaggccaacactgaagatgacaagaccgaggaggacgtgcccatgcccaagaactacctgtggctcaccatcgtctcgtgtttttgccctgcgtaccccatcaacatcgtggctttggtcttttccatcatggtgagtgaatcacggccagaggcagcctgggaggagagacccgggcggctttgagcccctgcaggggagtccgcgcgctctctgcggctcccttcctcacggcccggcccgcgctaggtgttctttgtcctcgcacctcctcctcacctttctcgggctctcagagctctccccgcaatcatcagcacctcctctgcactcctcgtggtactcagagccctgatcaagcttcccccaggctagctttcctcttctttccagctcccagggtgcgtttcctctccaacccggggaagttcttccgtggactttgctgactcctctgaccttcctaggcacttgcccggggcttctcaaccctcttttctagagccccagtgcgcgccaccctagcgagcgcagtaagctcataccccgagcatgcaggctctacgttcctttccctgccgctccgggggctcctgctctccagcgcccaggactgtctctatctcagcctgtgctcccttctctctttgctgcgcccaagggcaccgcttccgccactctccggggggtccccaggcgattcctgatgccccctccttgatcccgtttccgcgctttggcacggcacgctctgtccaggcaacagtttcctctcgcttcttcctacacccaacttcctctccttgcctccctccggcgccccctttttaacgcgcccgaggctggctcacacccactacctctttaggcctttcttaggctccccgtgtgcccccctcaccagcaaagtgggtgcgcctctcttactctttctacccagcgcgtcgtagttcctccccgtttgctgcgcactggccctaacctctcttctcttggtgtcccccagagctcccaggcgcccctccaccgctctgtcctgcgcccggggctctcccgggaatgaactaggggattccacgcaacgtgcggctccgcccgccctctgcgctcagacctcccgagctgcccgcctctctaggagtggccgctggggcctctagtccgcccttccggagctcagctccctagccctcttcaaccctggtaggaacacccgagcgaaccccaccaggagggcgacgagcgcctgctaggccctcgccttattgactgcagcagctggcccgggggtggcggcggggtgaggttcgtaccggcactgtcccgggacaacccttgcagttgcgctccctcccccaccggctcacctcgcctgcagctgggccacggaactccccggccacagacgca

SEQ ID NO: 146 - DNA, 600 bp, Homo sapiens
  source 1..600: mol_type = other DNA
ctctctgggccttaggaaaatggaaatgacacctgtacctgcccttccaggactgacaggaggggctgctccatgaaacctcactgctgcggtcataatgtcattatcttttgccttaaagggatttcttctgcaccagcacctaaagtggcagccccttacccttggccatcagctggaccctggtgctctcctggagcccaaaacctctgttttgtgttgcatcctgctgaccagccacagtccacacccatctgagtgtctgagcagaacagcccagaggccacaccaggatggctttccaccggtcaccttcccccacccactcataaaccctgcgtctctgggggagagggtggcgaggtcccctccccacatagatggaaacactgaggcctgattcatggtgccccctgtgaagcgcctcatggccagcaccggggggcagcaggccagggcggggacacatacccggttctcgtcgtagatgatctgcaccaggctgcggtgcttcgactcgatgggcggcggtgacacgggcttctcaggctcgggcggcttggcagcctcctcctccagctgttgctgtggggagaggca

SEQ ID NO: 147 - DNA, 400 bp, Homo sapiens
  source 1..400: mol_type = other DNA
cttgaaaactcccagccccctttgtccagatggggatggaggtggccaggctgccccgttgattgtgtgccgaggagccctccccgggaaggctgtgatttatacgcgcaggcttgtcacggggtgaaaggaagggccactttttcattttgatccaatgttaggtttgaaagccacccactgctgtaaactcagctggatccgcgggccgtgattaaacacattgcccgctttgttgccgagatggtgtttcggaaggcgctgtgaatgcacttccctttgcggggctcacacagacaagatgtgtgttgcaaggatgaggcgcctgctcggcctccagcccagggccgggaagggagaaggtgctgtgcgtcgctgcctgtgtcgcccgcggctctcc

SEQ ID NO: 148 - DNA, 1200 bp, Homo sapiens
  source 1..1200: mol_type = other DNA
cgcgtcagggccgagctcttcactggcctgctccgcgctcttcaatgccagcgccaggcgctcaccctgcagagcgtcccgcctctcaaagaggggtgtgacccgcgagtttagataggaggttcctgccgtggggaacaccccgccgccctcggagctttttctgtggcgcagcttctccgcccgagccgcgcgcggagctgccgggggctccttagcacccgggcgccggggccctcgcccttccgcagccttcactccagccctctgctcccgcacgccatgaagtcgccgttctaccgctgccagaacaccacctctgtggaaaaaggcaactcggcggtgatgggcggggtgctcttcagcaccggcctcctgggcaacctgctggccctggggctgctggcgcgctcggggctggggtggtgctcgcggcgtccactgcgcccgctgccctcggtcttctacatgctggtgtgtggcctgacggtcaccgacttgctgggcaagtgcctcctaagcccggtggtgctggctgcctacgctcagaaccggagtctgcgggtgcttgcgcccgcattggacaactcgttgtgccaagccttcgccttcttcatgtccttctttgggctctcctcgacactgcaactcctggccatggcactggagtgctggctctccctagggcaccctttcttctaccgacggcacatcaccctgcgcctgggcgcactggtggccccggtggtgagcgccttctccctggctttctgcgcgctacctttcatgggcttcgggaagttcgtgcagtactgccccggcacctggtgctttatccagatggtccacgaggagggctcgctgtcggtgctggggtactctgtgctctactccagcctcatggcgctgctggtcctcgccaccgtgctgtgcaacctcggcgccatgcgcaacctctatgcgatgcaccggcggctgcagcggcacccgcgctcctgcaccagggactgtgccgagccgcgcgcggacgggagggaagcgtcccctcagcccctggaggagctggatcacctcctgctgctggcgctgatgaccgtgctcttcactatgtgttctctgcccgtaattgtgagtccccgggccccgaggcagcagggcactgagactgtccggccgcggatgcggggcgggaagggtgga

SEQ ID NO: 149 - DNA, 2000 bp, Homo sapiens
  source 1..2000: mol_type = other DNA
cttccgccgcggtatctgcgtgcccttttctgggcgagccctgggagatccagggagaactgggcgctccagatggtgtatgtctgtaccttcacagcaaggcttcccttggatttgaggcttcctattttgtctgggatcggggtttctccttgtcccagtggcagccccgcgttgcgggttccgggcgctgcgcggagcccaaggctgcatggcagtgtgcagcgcccgccagtcgggctggtgggttgtgcactccgtcggcagctgcagaaaggtgggagtgcaggtcttgcctttcctcaccgggcggttggcttccagcaccgaggctgacctatcgtggcaagtttgcggcccccgcagatccccagtggagaaagagggctcttccgatgcgatcgagtgtgcgcctccccgcaaagcaatgcagaccctaaatcactcaaggcctggagctccagtctcaaaggtggcagaaaaggccagacctaactcgagcacctactgccttctgcttgccccgcagagccttcagggactgactgggacgcccctggtggcgggcagtcccatccgccatgagaacgccgtgcagggcagcgcagtggaggtgcagacgtaccagccgccgtggaaggcgctcagcgagtttgccctccagagcgacctggaccaacccgccttccaacagctggtgaggccctgccctacccgccccgacctcgggactctgcgggttggggatttagccacttagcctggcagagaggggagggggtggccttgggctgaggggctgggtacagccctaggcggtgggggagggggaacagtggcgggctctgaaacctcacctcggcccattacgcgccctaaaccaggtctccctggattaaagtgctcacaagagaggtcgcaggattaaccaacccgctcccccgccctaatccccccctcgtgcgcctggggacctggcctccttctccgcagggcttgctctcagctggcggccggtccccaagggacactttccgactcggagcacgcggccctggagcaccagctcgcgtgcctcttcacctgcctcttcccggtgtttccgccgccccaggtctccttctccgagtccggctccctaggcaactcctccggcagcgacgtgacctccctgtcctcgcagctcccggacacccccaacagtatggtgccgagtcccgtggagacgtgagggggacccctccctgccagcccgcggacctcgcatgctccctgcatgagactcacccatgctcaggccattccagttccgaaagctctctcgccttcgtaattattctattgttatttatgagagagtaccgagagacacggtctggacagcccaaggcgccaggatgcaacctgctttcaccagactgcagacccctgctccgaggactcttagtttttcaaaaccagaatctgggacttaccagggttagctctgccctctcctctcctctctacgtggccgccgctctgtctctccacgccccacctgtgtccccatctcggccggcccggagctcgcccacgcggacccccgccctgccccagctcagcgctccctggcggcttcgcccgggctcctagcggggaaaaggaaggggataactcagaggaacagacactcaaactcccaaagcgcatgattgctgggaaacagtagaaaccagacttgccttgaaagtgtttaagttattcgacggaggacagagtatgtgagcctttgccgaacaaacaaacgtaagttattgttatttattgtgagaacagccagttcatagtgggacttgtattttgatcttaataaaaaataataacccggggcgacgccactcctctgtgctgttggcgcggcgggagggccggcggaggccagttcaggggtcaggctggcgtcggctgccggggctccgcgtgctgcgggcggggcgggcccggtggggattgggcgc

SEQ ID NO: 150 - DNA, 1000 bp, Homo sapiens
  source 1..1000: mol_type = other DNA
agtttggggagccttttctccatttgagaaaaaacaaacttacagcgaggggtgaggggttagggtttgggattggggaaaatgtgggtggggagcccccccaaggaagtgaggagggggctgcaaggattacacctgggcatacgtttccctagaaatcacattcattgtatttttataatttattctaaatctttcatgcgaagaaagtcagtagtgagtgttagtactggtggccctcctgatcacacttgcatctcttgagtgtgccttaaaggtcttgggaatggaaaatataaaaactgcttcgtgatgcgtcatctttatcccccactcccccacccattccaatatattttctacttccagcctaaattcggggccccctaccgaggccggccatgatcttgagggcggcataggggaggccgcgctctgtccaccccagcctggtgatgccgttcgcttcttgtgcccggtattgtgggctacatgcctttccggcgtacggagctgagcgtccaggccagtgcccctcaacctctcagtaatgtttacccgaggccgtcgtgcaatgagactattcgcatggcattgtcaacgcggcggcgcgcgcgtctcggccctccgcggcttgccagactgtcctgcaaaccacctcacccgtctctttggcgcaggagactcaggctgtaaccggagaaaacacttcaccctggaaccctaactcaggtcctggcaaaagatgcgagaggaagacttgctctcttaataaatctcggccgcccgcacatctggcccctagacctgctcggtagaggactggctggtggatgcgcggtccaggccgtgggcactcgacccacctctattttccttcccgaggcgcccctggattaccactttcggtttgcgcttacatccgggatgtcgaatttcccagggaatcataattattttatctataatttattctaaccccaaggttccaagaaaatct

SEQ ID NO: 151 - DNA, 1100 bp, Homo sapiens
  source 1..1100: mol_type = other DNA
acattccttctaaaatgtgggctttctgtgtacatgggcgcgcattcccaggactcggttccctgggtggaattcacccaggaatacaatcgattttctgaacctgcgtaaggccacaggcagctctgaaaatgaaagcgtttgctaagtgggggagatctcaccgatcgaacgtttaaaaatggctttgtcttcattcagctctcccgatttattctgtgttttacaaatagaagctcagagcttctgtcgcccagtccttgcatgactcatggcggtggccacacgggtttcagggataacgggatgtttagaaaatcgctgcatatcggagtttcctagcacgttccatttatactgaacgcaggcggccgctgaaaatccagcctcgactcttgctaatgactgggtaggaccctcggggtcctgcgacggtgctggagggtgttcccggctccgatgtggggaggcctgcgcggggactaggttctcgagaggcgagcgggcgcgccagagaacccgagactgctgcggggccggatgcgggatccctgggctgcggttctacgcagaaacgccaatggccatgcctccccagctcctcccagccccagtcactaggccggcgcctggcccggagatcctcccagagccctggcggtgccatcatgccggagaagacaagctcggccccgctggaattcgctccaaacacagatgctcatttttggaatattctagaaaaataacaagatcttgtttgtcgttatgattcacgggaggtaactgatgggagggccatttacatgagggcagacactgtggggcgaaggtgacttctggacgtaggctttaaagtaggaacggctccaaattcccaatatctccggccttaccggttgcaaatcggacccctgcgggaaaaccagacacttctgtttcgtggctttcgggctgcctccagcccacgcaggctcgtttagtccccgtggagtcagccccgagccttcctagtcctggaacaagggctccaggtcgcggccgcgggaagccgccaagagggcggggagtagggattccctccagctccgcagggcatc

SEQ ID NO: 152 - DNA, 1500 bp, Homo sapiens
  source 1..1500: mol_type = other DNA
tcctcctcggcctcagatgtcgtcccacctgcccacgagcagggaacctggaacccactctcccggcagtccccagcgggttccgccacccggcggccgcccctgacaccgagtgggtgggaggaagaggcagctggcggggatgggccattgagacctcttgaaaaatattaaaagacaggatgggtagagatttctccgggagaaagttcgagggtgcatcgggtcgcggctgggaggagtacccgaaatgccagcaggagaaatgcaacctgtttaggccacaccttcaatccccgaggctgtctggagagactgcgtgcgggggacttgccggcgttcccacaccgcgcctgcaatccactcccgcggctgcctggcctctgccactcgcggcttgaagccagtggctctcaagccctcggccccgcggcggcccgcgcagccttcacccggcgccggcaccacgaagcctggccgcagtggactccccgcagctcgctgcgccctggcgtctcccgtcgaggagggagggacggaggcctgagccgggagctccctggcggtggtcgggccgccccccttgaggcctgctcccccctctcggcctcgccaaatccctgaaagcccagtcccccttcgtcaccccgggggcttctaatcactcggtatcgatttccctaactcttttcatcctgttgaagacacatcttaaaacactccagcccggagtgtgctctgggctttatccacactaataaaatgatttacccttctctccgcgctctcctcacagaggaaaatcgttcgagccccggctatttgtgtgtgatcagtaaatatttagtgcgctgacatccttagctgggcttcggatcgattcggggcccaccgggaggtgcgcacggtccgggcggggccgcgccgagctcgccgagggggctcctcccgccctcgccgccggccgctgatttacggcccctgcaaccagctaaggggggcgaaagcgcgcctggaaaattggcttttcaaccttttacttttgacattcagccacttccccaggctctaattctcgcccgcactcctccctcccgccctactaagggttgccctgtgcgccctgcgagcccttccagcagcaacgcgcggcgctcgcgccccctcggcccggggaccacctatcacagccctgagccgcgacgcggggaggccccggcccctgctatgggggtcgcctccttcgaggagagatgctctccgcccgcccacacctctgagggaggagagggggtggagaagcccagagctgcatctgctggatgacgagccgctctccctgctaccctttctccgacccgtcggcctttctcctactctggagactgatcctcgacgtccatcgggccggatggcgtcgggtggaagcgttactttcctcgcagaaaaactcctcctctttcctaagatcagaaaaagcgcttagcttggaattgttag

SEQ ID NO: 153 - DNA, 600 bp, Homo sapiens
  source 1..600: mol_type = other DNA
cctaggcattctcagcccgttttgctggagggggcatttgaggcctggccagcttagccagcctacaaggagtgttactggggtgaaaacagccagcggggaccagtctgcttgtggcccgccaggtgcctgggatggggaagcagcaaatgcccaccttcctgcccaaccccctcctccctcttcatggggggaactgggggtggcagcggctgccgggtgcgagcgggctcaggcctgtggccctgcctgacgttggtccccatcaagccatgtgacgagaccaggccacaagaaagaggtttcaacaagcgttatcgtttcctggaactccaactcggcgacttccccgaagaccggctgtgcctggcgggcgggctgcgcacagcggggacaaggctgcccccttcctcctccgctgcctccgcggccgcgtctatctcagtctgactacctggaagcagcactccaccctccagcccagcggccctcggctcagctgccaggtcaccggcaaccccgggagcggtggggcaggggctgctccgccagcctctgtgatgttcaggccgggctgcaccagcccgggacccctaggtg

SEQ ID NO: 154 - DNA, 700 bp, Homo sapiens
  source 1..700: mol_type = other DNA
gcactggttcccctttacctgagccaacaacctaccaggaagtttccatcaagatgtcatcagtgccccaggaaacccctcatgcaaccagtcatcctgctgttcccataacagcaaactctctaggatcccacaccgtgacaggtggaaccataacaacgaactctccagaaacctccagtaggaccagtggagcccctgttaccacggcagctagctctctggagacctccagaggcacctctggaccccctcttaccatggcaactgtctctctggagacttccaaaggcacctctggaccccctgttaccatggcaactgactctctggagacctccactgggaccactggaccccctgttaccatgacaactggctctctggagccctccagcggggccagtggaccccaggtctctagcgtaaaactatctacaatgatgtctccaacgacctccaccaacgcaagcactgtgcccttccggaacccagatgagaactcacgaggcatgctgccagtggctgtgcttgtggccctgctggcggtcatagtcctcgtggctctgctcctgctgtggcgccggcggcagaagcggcggactggggccctcgtgctgagcagaggcggcaagcgtaacggggtggtggacgcctgggctgggccagcccaggtccctgaggagggggccgtgacagt

SEQ ID NO: 155 - DNA, 300 bp, Homo sapiens
  source 1..300: mol_type = other DNA
tgtccgacaggcacacagagcgccgccaggcacggccctcattcttcaccccgagctcccgcaaggtcggcgaggaggctggagcagcgggtaggaagcgggccgaggctcccccgacgctgggccgcaactgtcatcgcagatccctgaaaaacgagctctgtaatcgttgccgtcagcgggtgtacaattgcagccttatgtttcctgccgctgtttaccttcctgagcggcgcccagagatgcacacacgctgccctgaagcgggacgtgacctctgggcacctgtgaggtcctggg

SEQ ID NO: 156 - DNA, 500 bp, Homo sapiens
  source 1..500: mol_type = other DNA
gtcggctcctgcgctcccaacggggtggccgtttccttcctcgcaccctcttctctcccggtgcctgcggtcccaccttccagatacccctcggagagtccagctgagctctcgccagagctttccccttccaacccgctcgacttgcccagatcccaagctgggcttctctctccatcgccccagaaagtgggtcttggagaccgaggcaagaatttgggcctccgcttctgttccagaccccggaccccttgccaaaatgcggcagatgtgcagattgggccgcgcttggttcctggctgggtttatggagcctgcggctgaggcaggctccgcagaccccgagccagagtgggatttaacggcggccggtgcgctgtgcttggtcaaccccggtaaccgtcacgctgctagtgatatgaaaaaaacctgccagcgttctgcttttctgccccgctgcagtctttagcacccgccaggattctgtccgagtgtttgga

SEQ ID NO: 157 - DNA, 1000 bp, Homo sapiens
  source 1..1000: mol_type = other DNA
tttagtgtgtgcataaaacatcccagctaatctcaaatagacttttcctgagcagaggctgaaatttgcaagtaatgcaaagaagactccgggagagcgtcgccgatggtggagcgggagacgggcgtggggagccccactgcagtgctgggatcgaagtggtgctgaccccaagacctctcccctcctcctcccccgggagcttctccagggttatttgggaaatgagggggaactccaatccctgagaaagcgctcaggggcttgctgaggtgagcgcaaatggaagcacaaggccgggctggccgtgggctcagtaaccagtcggctgcccggcttgcgccagcactaaatgctcgatcagaaagagaaaaagaggcgcaataattccaaatttcaggaaaagtcaaatcggagaggggggacgcaggtctcttcagactgcccattctccgggcctcgctgaatgcgggggctctatccacagcgcgcggggccgagctcaggcaggctggggcgaagatctgattctttccttcccgccgccaaaccgaattaatcagtttcttcaacctgagttactaagaaagaaaggtccttccaaataaaactgaaaatcactgcgaatgacaatactatactacaagttcgttttggggccggtgggtgggatggaggagaaagggcacggataatcccggagggccgcggagtgaggaggactatggtcgcggtggaatctctgttccgctggcacatccgcgcaggtgcggctctgagtgctggctcggggttacagacctcggcatccggctgcaggggcagacagagacctcctctgctagggcgtgcggtaggcatcgtatggagcccagagactgccgagagcactgcgcactcaccaagtgttaggggtgcccgtgatagaccgccagggaaggggctggttcggagggaattcccgctaccgggaaggtcggaactcggggtgatcaaacaa

SEQ ID NO: 158 - DNA, 500 bp, Homo sapiens
  source 1..500: mol_type = other DNA
catggtgcttcaggaagggaggggacgagagccctgggcttgtggtgtccacgtggacagctaatgaggagccttgccgatgaggagcatgcgttcccgacggggcggccgaatgcggaaggagccgccattctctccgccctgaccgcgggattctctgcagcagatgagaaacggcgctgactcagcagggtccctcccaggccccgagcggtcatctggtgacccccgcgcttcccccacggcccagccggagaagggcaaagggaagtcccggctccaaggcgcacccagagatgcggtgcatgtggcaggatggcccagccccgtcggcagccccagcttcctgcccctggtttccttcctcccacgggctacaggcctctgatgagctttggaaagcaggaaacacacaggctagtaactatgaatgggtccaaaaaacactccttattactttaaactacttaggaagaagcacagcgttgccaaacgccaga

SEQ ID NO: 159 - DNA, 1200 bp, Homo sapiens
  source 1..1200: mol_type = other DNA
gcgcggggggccggaggatggcggcctgggggccctgcgggggctgtcggtggccgccagctgcctggtggtgctggagaacttgctggtgctggcggccatcaccagccacatgcggtcgcgacgctgggtctactattgcctggtgaacatcacgctgagtgacctgctcacgggcgcggcctacctggccaacgtgctgctgtcgggggcccgcaccttccgtctggcgcccgcccagtggttcctacgggagggcctgctcttcaccgccctggccgcctccaccttcagcctgctcttcactgcaggggagcgctttgccaccatggtgcggccggtggccgagagcggggccaccaagaccagccgcgtctacggcttcatcggcctctgctggctgctggccgcgctgctggggatgctgcctttgctgggctggaactgcctgtgcgcctttgaccgctgctccagccttctgcccctctactccaagcgctacatcctcttctgcctggtgatcttcgccggcgtcctggccaccatcatgggcctctatggggccatcttccgcctggtgcaggccagcgggcagaaggccccacgcccagcggcccgccgcaaggcccgccgcctgctgaagacggtgctgatgatcctgctggccttcctggtgtgctggggcccactcttcgggctgctgctggccgacgtctttggctccaacctctgggcccaggagtacctgcggggcatggactggatcctggccctggccgtcctcaactcggcggtcaaccccatcatctactccttccgcagcagggaggtgtgcagagccgtgctcagcttcctctgctgcgggtgtctccggctgggcatgcgagggcccggggactgcctggcccgggccgtcgaggctcactccggagcttccaccaccgacagctctctgaggccaagggacagctttcgcggctcccgctcgctcagctttcggatgcgggagcccctgtccagcatctccagcgtgcggagcatctgaagttgcagtcttgcgtgtggatggtgcagccaccgggtgcgtgccaggcaggccctcctggggtacaggaagctgtgtgcacgcagcctcgcctgtatggggagcagggaacgggacaggcccccatggtcttcccggtggcctctcggggcttc

SEQ ID NO: 160 - DNA, 600 bp, Homo sapiens
  source 1..600: mol_type = other DNA
gggcgggttgccacactgtcccctttctgcatgggaggaagggggctcgagaactgagtcagccacacaaaacgaggatggacagaactcctgagtagcgagggtgcctgccgggcgcgaggaggagggggaagacgaggaagacgaggaggaggaatagggagcaccacatgacagaggggctgcctcagaccacaaagcgcttcctcatcctttcctcgccctttgatgccgccggcaacgtgactctgcgagcagcggggcagacgccaggtctccctcgcaggcgggaaaggggctccaaggcgggtgctgccttgctcgggtcacatggctacgtgggggccttgctcaaattcacttcctgccttcattacaaaactgtcaaaggggatcgcacgtttgcagggtgtcacccaagcattctggttttgcaaacgacgctgtgcggcaggcggtctgatacctgatgagctcggtgtggcggggtcggcagcatttcctccggggttttgagctctggccacttctccttttgttccacccaatctcacccacttctgggcttcgaggccagagtgtcttaacaagggggcacgt

SEQ ID NO: 161 - DNA, 500 bp, Homo sapiens
  source 1..500: mol_type = other DNA
gagcgagactttgtctcaaaaaaaaaaaaaaccaaataaattgaaagctgagaaattcagagcacaagaagacaagcgcgccccctcttttagctgtcaacatggcggagccgtccctggtgacgcagcctccaaaggcctccctgtgccctcctgagaccgcaagagggaaagtggcagcgacagtgatcgtggtgtctttgtggcggttgtgttgacctcactgacccccgaagtgccgctctagggtctgtcctcagcggtgacccggccgggtcgaagggcagagttccgctgtcactagccctccacccgtcctgtgtgctgggatgccctcgcggcgccgtccacgccaccgccgccccctcttgtgggttctgtctcctccgtgtctaggatcctcctgcatccgtttttccttcctcccttctctccctccgtctgtcttgcccgcacctgaggttgtcgcagaggcgctgagacgggccagcaggagctgt

SEQ ID NO: 162 - DNA, 600 bp, Homo sapiens
  source 1..600: mol_type = other DNA
tgctgtcccggtcctgtcgcagtcctcaaagatgctagagtgacagtcctctaggggtagagatggtcgtcctcccaggagaaggtggcccggagacttggaggtgggatcaatcctgccagtcctggatcaggaggcctctgtcgggcgccgccccccttcctcctccatcagcaacaggcggcgccggccagcctcatagtcagcctcatccacactgaccagcaggcgaacagcctcccggcccacagcctctcgcagggcctcagtcaggaacacgccccgcagggcctgcagcagggcgccactcaggtagtcgccccagaaggcgtccagataggagagctctgagaacttgatgtcacaaaccacagagcccaggtcccttgagcgcagcactgcggtggcctgcccaaacacgtccagctgccgcgccagcgcctggggccgccgggatgccacgccctgctccaaggctggcccatgctcgcagtactctgctcgaacccggagccggatgtctgcaggggaaggagggatttgtcagggagggggccaacactagacacacttatggggaacgccacccttcctccctcc

SEQ ID NO: 163 - DNA, 500 bp, Homo sapiens
  source 1..500: mol_type = other DNA
tgatgcccggcccccaggggggcagaggcgccgccaccatgagcctgggcaagctctcgcctgtgggctgggtgtccagttcacagggaaagaggcggctgactgcagacatgatcagccacccactcggggacttccgccacaccatgcatgtgggccgtggcggggatgtcttcggggacacgtccttcctcagcaaccacggtggcagctccgggagcacccatcgctcaccccgcagcttcctggccaagaagctgcagctggtgcggagggtgggggcgcccccccggaggatggcatctccccctgcaccctccccggctccaccggccatctcccccatcatcaagaacgccatctccctgccccagctcaaccaggccgcctacgacagcctcgtggttggcaagctcagcttcgacagcagccccaccagctccacggacggccactccagctacggtgagggcctgggccatcttggcccacttttcaga

SEQ ID NO: 164 - DNA, 200 bp, Homo sapiens
  source 1..200: mol_type = other DNA
ggccgggcaaaaagccgccgcaacaaaaagctgcgctgacgggcggaaaaagccgcggcggcggagccaaaaagccggggcggcaaaaagccacggtggcgggcgcaaacagccgcaaaaagccgcggtggtgggggcaaaatcagtgggagcaggggcaaaaaaacacaaaaagccgcggcggcgggggcaaaaagcca

SEQ ID NO: 165 - DNA, 400 bp, Homo sapiens
  source 1..400: mol_type = other DNA
tggctttgctggagtgtgatgtgataggaaatgtgcagccaaagacaaaagaagatgtaagtaggcttgactcattgcagctaagaacccagatgttaccttgagggtattaactaataagcagtttaaatcagaatggcacattctgatttgttttttgtatgttcacatttggcaggcatagatactgtttgaaaagagaaaagtcagtacatagaggtaacaagcttaaatatgtgccaagtctagaaacaagagactagggggataaggacctttcgaaattaaatgcaagatttgaaaactgattggctgggggatgaggcaaaggcaggtctttaaggtcaatccctgttttgctttaagttgttagcgggtggttttatcatatattgtagaa

SEQ ID NO: 166 - DNA, 650 bp, Homo sapiens
  source 1..650: mol_type = other DNA
ttcctgggaatgtcagctaacctgagcctaggggcctgagcccaagggcagactgaggctcccccagcacagggaggtgctgcctgtgacaaggggtagtgctggcacagtgcaggctactccctagaaagatcagcttgaatatgcaggaagagcaggaccctcgggctgaggcagaggtggaatgggaagtgcatggtggtaatttagttctccagaggccagaagtaggaggagcggttggaatgctgatggcccaaagggaaaccctggactaccctggcctcccacaggactctcatagtaattgcggctccctgcagtggtgaggccagaaggagtgttgcccaatgctgtcatcatccagtccaccccccacccaccatcaacagatgagtatggtcatgagtgtggtcacctcatcagtcatttgctcagttgtgaaaaagaaattgttcagagaagagcaaagtgtttttccatgagccaaaggtcagccaagttatgctaatgaggaggactggagacagcgtgtcacagacaccgagaaggagcactgggcaagggcacttctcccagggcagagcccacaagaagcgtcctggcaccagacactcagggaactgaaggctggcaggggcccgcccagt

SEQ ID NO: 167 - DNA, 5000 bp, Homo sapiens
  source 1..5000: mol_type = other DNA
tccccccagctgggtataagcaaactttcctgtctatgggccgcagagaccaccatctagttcccccgccaaaactttacatgattttaattctcctgatgaagatgagaggataacagccaacagagagggcagaggatgggatgggactcccttgctcagagacctcacctctaggtctttacctcctattgagaataagtcagttctgtagtaagaactctgtgtccacggcaaccccaaacagaatcctagcgctcttgtgattcttgtagaatggggaatagaacgagcttggcccaagactgcacagacttaaaaacatactattctttgaaaatggcaatcattaaaaagtcaggaaacaacaggtgctggagaggatgtggagaaataggaacacttttacactgttggtgggactgtaaactagttcaaccatggtggaagtcagtgtggcgattcctcagggatctagaactagaaataccatttgacccagccatcccattactgggtatatacccaaaggactataaatcatgctgctatacagacacatgcacacgtatgtttactgcagcactattcacaatagcaaagacttggaaccaacccaaatgtccaacaatgatagactggattaagaaaatgtggcacatatacaccatggaatactatgcagccataaaaaatgatgagttcatgtcctttgtagggacatggatgaaattggaaatcattctcagtaaactatcgcaagaacaaaaaaccaaacactgcatattctcactcataggtgggaactgaacaatgagaacacgtggacccaggaaggggaacatcacactctggggactgttgtggggtggggggaggggggagggatagcattgggagatataccaaatgctagatgaggagtttgtgggtgcagcgcaccagcatgtcacacgtttacatatgtaactaacctgcacattgtgcacatgtaccctaaaacttaaagtataataaaaaaaatactgttctgccatacatacagatactcattaaagatgagggagaagggcatggggtgggggagaatgtaccaaaaccaaagaccacaggataataacctcagagcagagactatctctctagttattttttcttttgtatgtaatggagaggattattatttactctgatgaagaagtttacatcaagtgttcagcttcctttgtgggttacagagaataaccagagggctcagttatgctctctgaataactatgtttgcttagtgttttctaaacaatattaaatttcactaaaatagacaaggttgataggacttgggggcataactcattgactcaagctatcattttataggattgtgagaaaacaaatagatgaacatttaaaatacactcatattctcgctagaaaagaggattttgaatattcttacatcaaagacatggtaaatgtttaaggcaatgaatatgctaattaccatgatttgatcattatgcaatgtaaaatgtactgaaacatcacattgtacctcataaatatgtacaatttattatgtgcgaattaaaattttgagtataagaaaaaataaacttcaattgtaagaaaacaacccaacttttaaaaaacgggcaaaatacgtgaacagatacttcactaatagagatttgcaactggcaaataagcaaatgaaaaactggtcatcatcactatctattagagaaatgcagattaaaactacaataagaaacaatgctgcccgtccagacgcattgttttgaccgtttccaacttgtcccagcccttcccggggcatcgctggggaccctacgccgacgtcccccctccgcccgcgccccaagggccgactgggcaaattgggagacccgccccgcggggcgacccaacttttcggaacagcaccccaccgcccacccccgcagacccccggacccccgctcccggcggagactcagggaaccccgcaccccaagcccttctaaatcgtgcagcgtgagtgtgacggccaagagcggatgcagcccgggatcgcccgcaccttcccgtgggcggaagcgcaggagccagctggggagggggcgccctagaggagcggctagaaagcagacacggggaactcaggtcatcctggggggggacaagacaacgagagccgggcgcctcgggggcggcgcgggagcctccgcaggaccgggcgggcgccccggctggcgcgggcggggggcgcgccccctttacctgcggctccggctcctaggccatttcctcacgcggcggcggccgggactgagctaacaccactcaggccggccgggtttgaatgaggaggagcgggcgcggagaggaggggacggggagggcggagggagggagggaggcgtcgcggagtttttctcggccttttgtgcggacacctcccggattccgcgcccgcacccggccccccaaaagacacggggagccgcgggcgaggggttcagccatccgccgaggcgcctagtgccttcgcgcctccaagacccccccccaacaaaaaggagcgtcccccacccctacccccgcccggaggacttagggcctgggctcacctcgggcgcggagctaagtgtaggcgccgggggtccctagagccgccggggcgcagcgagtccggcgctgggtaactgttgggtcagaaactgttcaggtagcagctgttgtgccctcccttggccccgccgctcggagacgccccgccccctgccttgaacggccgcccggccccgccccagcgcccacgtgactagcataggcgcgcccccgttccgcccgccgccgcagactccgcctccgggacgcgagcgagcggcgagcgcgcgcactaccagttcttgctcggcgactcccgcgcacgcgcgcgccgtgccaccctccccgcacccctcctcccgccatccggcttaacgtggcgggcgcgcgccgcggcagtagccgtgacaggtacccggcggggcggggggggagggggttggcccgcgagggtgtgcgcaggcacagacccgggtcctgtccccgccgccccctcctctgcaaggtgtgcctgggcgaggggaggggcccgcggcccgaacccctgggtcacccccgaattacaaacaaaaaccttaacgccattgctcgcgggttagaaggcagctgtgcgtgctcaggaaaagaagccacgcacaagagaccgcacgcggcgtggatacagtgacacgaaacacccaaaatctcttttgaaagggaaaccaggcacagtggctcatgcctataatcccagcactttcgggggccaaggcgctcacctaaacccgagagttcaagaccagcctgggcaatacagcgaaaccctgtctctacgaaaaatataaaaattagctgggcatagggctgggcacggtggctcacgcctgtaatcccagcattttggaggccgaggcgggcggatcacgaggtcaggagttccagaccatcctggctaacacagtgaaaccttctctctactaaaaatacaaaaaaaattagccgggcgtggtggcaggtgcctgtagtcctagctacttgggaggttgaggcaggagaatggcatgaatcagggagcggaggctgcagtgagctgagattgcgccactgcactccagcctgggggacagagtgagactccgtctcaaaaaaaaaaataataattagctgggcatggtggctggcacacatggtcccagctactcaggaggctgaggtggaaggatctcttgatcccggggaggtcaaggctgcagtgagccaagatggcatcaccgcactccagcctgggccacagaccctgtctcaaaaaaaaaagagaaagtggggaagaaaatgtaatacaaattaatataccaacagcaattagtgagtactttttccatggagctgggagagggaataaatgtttgtaaaattaaaatgttctacgctagaaatcaactttccttctatgctttctttacttcaccccttatagctacttagtaaatctcacaaatcctatccttctgatctctctgaaatgtatgtaccctttcccttctattctcaccacccatgtttctttgtttccttctagcctgtgtaataatctcataatcgcacctcctgtacctgccttctttctagtccagaatacgttttcctaaattccaccaataaccatcctgctactgctttgtgtgaaattctccaaaaaaaattttacttttccaaaataagtcaggctccctctcttaggatacaaaaccacaccatggtcccagccaatctttcagcctgattcactcagtatatatttattgacctctcctttctcccaagcacttggctagataataattaaagagtgcggcacaaaacaaattggattcctcccctcatggagcttgtattttcacaggaagcacagacattaaataaattaaaacacaaaaaaatagacaagcatataattacagtatgtatcctagagaaatatcactcatgcagaaagcatacacaaggatgcagcactgtttccaatagcgaaaagctagaaacaacctacatgttcaccaaaagaaaatggccacataaactataccatatccaaattatccaaattttagaatatagacaacaggttgggcgcggtggctcacacctgtaatcccagcactttgggaagccgaggcgggtggatcacaaggtcaggagttcaagaccagcctggccaacatggtgaaaccccgtctcctctaaaaaaacaaaaaaatcagctgggcactgtggcaggagcctgtaatcccagctactgaggagactgaggcaggagaatcgcttgaaccctggaggcagaggttgcagtgagccaagatcgcgccactgcactctagcctgggtgacagagcaagactccatctcag

SEQ ID NO: 168 - DNA, 1500 bp, Homo sapiens
  source 1..1500: mol_type = other DNA
tgtaggagtcctccggtgctggagtccagagcacagtgaggctgggtcctcccgtgccatagtgtagggcatggcgggacagggatcctgccctgcgatagtccagtgcttgagtccgcagtaaggcaatggtcctccaatgctggagttcacggcgttgtggggtcggggtcctttggtgacttagtccagggcgtaccagggcgggggtccacagttgccatagtgaggatcttggaggaaggtggttcctgccttgctgtagtccggggagcagggggcaggggtcctctcttgtcagagtctctggcgcggggtgggggtggaggtgggggttttcctatgcgatagcccacgggtcggtgaagccgggtcctcccgtgcctttgtccagggcgcaggggggcgagggtcttcggtggtggagtccgcggagcggcaggacgggggtcctccagtgccatattccagggcgcggcggagtgggggacctgtcctgcagtggtccagggcatgtgggagtggtggtcctgctgtgcctcagtccagtgcgcggtgggacggcggtcctgctgtgctgtagtgcaggacgcggtggcgcaggggtagtccagagagcgccgtggcagggggtcctccagtgctggaatccagtgcaaggcgggtcaggggtcttaccgtgccgaagtcggtggcaagggtcctcccgtgccatagtctagggggcgacggggcagggttctctagtgcaggtgtccagggtgtggcagggcaggagtcctcttgtgcaggagtccaggacgtagccgaggagtcctccaatgtcagagtccagggctctgcggggccgggttcccccatgccagagtgtagggcgcgttcaggtgagggtcttggcgtgcagtaatccagggtgcggtggggcaggggtagtccagacctccatggcgggcgtccctctgtgcaggagcccagtgcctggcggatcgggggtccttctgtgctgtagtccagggcaccgcaaggtgtgggtcctctggtgccctagtccagggggcggcgagtcagaggttctcccgtgtctcagtctagggcctggtaggactggggtcctggagtccacgtggtagcccaagttgccgcaggaccaggtactctggaaccacagtccagggcgctgaggggcaggagtagttcagggcgagccggggcccaggtcctcgggagccagagtccagggtgtggaggggtgggggttctgcagtggcacagtccaggacaccgcggggcgggacagggcggggatcctcccgtgccttagtccagggctgagccgcgggagaggtccttcagtagcacagtctagcgcacggcgttgcaggtgtcctccagtgcctgaggccacggcaggtcgcgggtcccactgtgctctagttcagggcggagtgggtctgaggtcttctcctgcctcagtctagggcgctggagagcggggatcct

SEQ ID NO: 169 - DNA, 2500 bp, Homo sapiens
  source 1..2500: mol_type = other DNA
gggttggtcctagaaagcgtgaggatcgccgagtgcactgccctcccagcctagggtccactcttccttggcccgagcccagagctcggggtttcaggcgctgggccctgtgcagctgcccagaataggctgagcggcaggttcccgccctggcaagggatccagcagtggaatcctcactgctgttggctgcgggcaaggtcagcggggtttccatcgctgctggtgggagccacctggcggtggtagctgcaagtgagcgcgtggcagagactggcagggctggtcccagacaccctgagggtctctgggtgcatcgccctaccaccctagggtctgctcttccttagcctgctcccaggacgcggtgtacgagggctagactctgagcagcctccaggatggggctgagcagcggattcctgccctgctgcagctacagtctgaattaggcgccaccgcagtatctggccctggggtacgtgctactgggtggcatggacagagatgggggctgccacagctgctatggggctgagcagccgattctcgccctgctgcagcgggcgaccgctgcaatccccagcgctatgggaccgaccacctgacttagatgccttggaggcatccggtcctggggtcttgctgctggtgtctgcgggcagggtcacggctgccactactactgctgtgcgccatgggcaggtgccagctgcagctgagtccgaggcagatgctgtcagggctggtctgaggttgcctaagggtggctgagtgcaccacgcttccaccccagggtccgttattcctaggccggctcccagattgcagggttgtgggcgttggacactgtgcagccatgaggatctggttgggtgcagattcccgccctcctgcagctgagaagccaatctcataacaggcgctgcagtgacctctggctctgcggtccgcgctgctgctggagctggcagagaacagagctgccaccgctgctgcttccaggagtgtgcagctggcagctgcagctgagcccgtggcggaggctggaaggccttattccagaagccttgagggtccccgaatgcaccgccctcccaccctaaggtccagtcttccttgcccgcgcccagagagttggattgcaggcgctgagcacagtgcaggtgctgggatggggctaagctgaaagtttccgccctctggctgctgcggggccgacagcctgagttatgcgccgcggcggcttttggtcatgggatccgcactgccggtggcttgcacagggtcgggggctgccacagctgctatagttcaccgtgtgcacgtggcagccgcccctgagcccaccgctgaggctgcagggctggtccggtcccagacggcctgagggccatttgcccgcgcccagatccgggtggctgcgctgggcactgtgcagcctcccggaatccgctgaagggcacgttcccgctctcctacagctgtgggccgactgcctgattttggccactaggtggagtctggctctagggtttcgaggccgctggtgttggtgggcggagtccgggtttgccaccgctgcgctccatgagcaggtagcagctgcagcggagctttagaccgaggctggcagggctggccccagacggcctgagggtcagggagtgcagggtcctcccaccctaggtccgctcttcctttccccttacccagagcgggttgtgcgggctctgggctctgtgccggcgctgggctctgtgcagccgccgagatggggctgagcagcggatttcctccctgctgcagctggaggacgattacctgcactagccgctgaggcggcatctggccctgggttactgcagctggtgacgcgggcagggtcagggttggttgcaggtggcagctgctgctaaacccattgcgagcctcagggtcaccaagttcaccgtcctttcatcatagtatctgatctttggcccgcgcccagagtgcggactggcctgcgctggggactgcatagcttctgggggccggtcagcgccagtttcacgtcctcctgcagctgcgtggcctaaggtcttaggcgccgcggcgctatctggccctgctgtcgacgctgctggtggtggggacagggtcaagggttgccactgctgctcccgtgcgccatcggcaggtggcagttgcagatgagcccacaattgaggctgttggggctgctcccaggttgttagagggtcgccgagttcaccgacatgccaccctaggttacgctcttggcccgcacccagagcgccgggttacgggtcctgggccctgtgcagccacggggatggtgctgagtgcaggttcccgtcttcctgagatgcggggcgaccactggaattagcctctgtggtggtatctgaccctagggtccgagctgctggtggcgtgggcggggtcgaagtcgcctctgttgctgcggcgtgccatttgcaccgtcctctggtac

SEQ ID NO: 170 - DNA, 1600 bp, Homo sapiens
  source 1..1600: mol_type = other DNA
aaatactctactgaaaaaacagaaatagtaaatgaatacagtaaagttttagaatacaaaatcagcatagaaaaatcagtcgcatttctatacccaacagcataccatctgaaaaaggaatcaagaaaccaatcccatttaaaatagctataaaaaaatgcctgggaataaactaagccaaataaatatgtctaaaatgaaaactataaaacattgataaaaatcaattgaaaaagatacaaataaagggaaagttatcccatttttatgaattagaagtattaatactgttaaaatgaccatcatactcaaatcagtctataggtccaatacaatctctaacaaatttccaatgtaattcttcagagatgttaaaaaaggttttaaaaatcgttctgcggatgttaaaaggatttttaaaacgcttttttcgttctgcaggcgaaggctgtggccgtgctcccgccggccagttcccagcagcagcgcattgcccctgctccacgccttcgctccaggcccgcaggggcgcagccccgcgggaatcagcactgagccggtcccgccgccgccccagtgtccgggctgcgactgcggggagccgatcgcccagcgattggaggagggcgacgaggccttccgccagagcgagtaccagaaagcagccgggctcttccgctccacgctggcccggctggcgcagcccgaccgcggtcagtgcctgaggctggggaacgcgctggcccgcgccgaccgcctcccggtggccctgggcgcgttctgtgtcgccctgcggctcgaggcgctgcggccggaggagctgggagagctggcagagctggcgggcggcctggtgtgccccggcctgcgcgaacggccactgttcacggggaagccgggcggcgagcttgaggcgccaggctagggagggccggccctggagcccggcgcgccccgcgacctgctcggctgcccgcggctgctgcacaagccggtgacactgccctgcgggctcacggtctgcaagcgctgcgtggagccggggccgagcggccacaggcgctgcgcgtgaacgtggtgctgagccgcaagctggagaggtgcttcccggccaagtgcccgctgctcaggctggagggtcaggcgcggagcctgcagcgccagcagcagcccgaggccgcgctgctcaggtgcgaccaggccctgtagctgtgacttggctgtggggctggcccgcctccctgacccctgtcaggcggagcagctggagctgacccacgggcctgggctttcgagcgctttgtccaggcgctaatgatgggaaggtgaaaggtgggggtggccacaccctgcagtcagggtggcaggtgtcagaggccacatgcaacccactggttttgtcttttccaggatgctgataagtttcccgcggcccccggagcagctctgtaaggccctgtaattgcctttcgttcccttctgctctattgaggagtgggaagatgacaaagtgtttttgctcaacccgaaggaaaatgcacatgggaggacacaccgggttactatttgagtagcccagacaggagagcagcggtctgct

SEQ ID NO: 171 - DNA, 1500 bp, Homo sapiens
  source 1..1500: mol_type = other DNA
tgggtggattgcttgagcccaggagttcgagaccagcctggacaaaatggcagaaactccatgtctacaaaaaatacaaaaattagccgggcatgatgttctgcgcctgtagtcccagctactcaggaggctgaggtgggaggatcgcttgagcccaggaggcggagtttgcagtgagctgagatgtcactgcattccagcctgggagacagagccagactctgtctcaaaagaaaaaaagaaaaaaaaaaaagaaaagaaaaaacgaaattgtattctgaatacatcttctaaaacactacatttacttgcactatattaaactggttttatcctgaccacaattgcaggtgaaagataccactgttgttctatttttctggtaagtagagtgagccatgtcttccccagggaaagacgcctcctaaaaatttgtaggaccacctttggttttcttccagatattttttttgtcatcgcttttcctgcgcccaattcccatctgtctagcccttctgcctccgctggtctttttcgcgagcctctccccagccgcaggtattcgtctgggctgcagcccctcccatctcctggggcgtgaccacctgtccaggccccgcccccgtccaacccgcggagacccgcccccttccccggacaccgggttcagcgcccgagcgtgcgagcgcgtccccgctcgtcgcccggctcggcgtcgggagcgcgctctgtgtggtcgctgctgcagtgttgttgtggctgtgagaaggcggcggcggcggcggagcagcagccggaccagactccctagtagctcaggcgctgccctgcgccggccctggcagggagcctggtgagatggtggaggaggaggctgtgccgtggctggccttgctgtgtcctgctgcctggttagaaccccatccccgtcccccgtctcctccggggggtgaggaggagctggaagaggggccggcctctgtccggcccggccaggcggcagtcaccctctgaggaggcagcgcccggggaggggcctcccaggcggccgccgccgccagggggaggcgctgggagtgggagtgggagcgggacctcagctgccaagctcggcccggaccctaggtgcgggggaggcggggtcccgggctcgggctgcctgcccggacctggcggggatgggcccgtgcggctccgggtgtgggacgtaccctcagagcgcccggggttattcccactgactccagggaggtgagtgtgcgcccttcgctccctgccgtgtctgtgagggtccatcgttgccggagactggaggtcgggggccatgggagccccggggcgaacggtgcggacatgggccttgtggaaaggaggagtgaccgcctgagcgtgcagcaggacatcttcctgacctggtaataattaggtgagaaggatggttgggggcggtcggcgtaactcagggaacactggtcaggctgctccccaaacgattacggt

SEQ ID NO: 172 - DNA, 1700 bp, Homo sapiens
  source 1..1700: mol_type = other DNA
gtctctaggacaccctaagatggcggcgagggagacggtgaaggttggctcccgcctgtctgggctctgatcctctgtctccccctccccctgcggccggctcatggcctggcggaggcccgaaccaaagacctccgcaccgccgtgtacaacgccgcccgtgacggcaagggggcagctgctccagaagctgctcagcagccggagccgggaggaactggacgagctgactggctaggtggccggcggggggacgccgctgctcatcgccgcctgctacggccacctggacgtggtggagtacctggtggacccgtgcggcgcgagcgtggaggccggtggctcggtgcacttcgatggcgagaccatggagggtgcgccgccgctgtgggcgcggaccacctggacgtggtgcggagcctgctgcgccgcggggcctcggtgaactgcaccacgcgcaccaactccacgcccctccgcgccgcctgcttcgagggcctcctggaggtggtgcgctacctggtcggcgagcaccaggccaacctggaggtggccaaccggcacggccacatgtgcctcatgatctcgtgctacaagggccaccgtgagatcgcccgctacctgctggagcagggcgcccaggtgaactggcgcagcgccaagggcaacacggccctgcacaactgtgccgagaccagcagcctggagatcctgcagctgctgctggggtgcaaggccagcatggaacgtgatagctacggcatgaccccgttgctcccggccagcgtgacgggccacaccaacatcgtggagtacctcatccaggagcagcccggccaggagcagctcataggggtagaggctcagcttaggctgccccaagaaggctcctccaccagccaggggtgtgcgcagcctcagggggctccgtgctgcatcttctcccctgaggtactgaacggggaatcttaccaaagctgctgtcccaccagccgggaagctgccatggaagccttggaattgctgggatctacctatgtggataagaaacgagatctgcttggggcccttaaacactggaggcgggccatggagctgcgtcaccaggggggtgagtacctgcccaaactggagcccccacagctggtcctggcctatgactattccagggaggtcaacaccaccgaggagctggaggcgctgatcaccgacgccgatgagatgcgtatgcaggccttgttgatccgggagcgcatcctcagtccctcgcaccccgacacttcctattgtatccgttacaggggcgcagtgtacgccgactcggggaatatcgagtgctacatccgcttgtggaagtacgccctggacatgcaacagagcaacctggagcctctgagccccatgagcgccagcagcttcctctccttcgccgaactcttctcctacgtgctgcaggacccggctgccaaaggcagcctgggcacccagatcggctttgcagacctcatgggggtcctcaccaaaggggtccgggaagtggaatgggccctgcagctgctcagggagcctagagactcggcccagttcaacaaggcgctggccatcatcctccacctgctctacctgctggagaaagtggagtgcacccccagccaggagcacctgaagcaccagaccatctatcgcctgctcaagtgcgc

SEQ ID NO: 173 - DNA, 300 bp, Homo sapiens
  source 1..300: mol_type = other DNA
taaaaataaattgtaataaatatgccggcggatggtagagatgccgaccctaccgaggagcagatggcagaaacagagagaaacgacgaggagcagttcgaatgccaggaacggctcaagtgccaggtgcaggtgggggcccccgaggaggaggaggaggacgcgggcctggtggccaaggccgaggccgtggctgcaggctggatgctcgatttcctccgcttctctctttgccgagctttccgcgacggccgctcggaggacttctgcaggatccgcaacagggcagaggctattatt

SEQ ID NO: 174 - DNA, 600 bp, Homo sapiens
  source 1..600: mol_type = other DNA
cgccaccacgtgcgggtagcgccgcatcgccccagccgtgttccttggtctccgtctccgccgcgcccgcctggtgaactggagcacagggaccatagttctggaaatttatcctttttctctccatggattcagcagcagtgtctaaaagaaaaaaattcatcaatcatttatgtatattttaatataaaggtaaaacactgcgaaccagtggaaccggatagaaagtaattcagttttacagaacacaactgtttttcaggctcttttattaaatataaaagagccatatatatttctgtggaattccccttttacttaagaattcattatcagcgaattagtttaaggaggctgttttgttagaggctgtggttgcattcaaaaattggaataggaacaatgacttgtaaaaattcaacattttattttatttttgagatggagtctcgctctgtcgcccaggctgtagtgcagtggcgcgatctcggctcactgcaacctcagcctcccgggtttaaggaattctctgcttcagcctcctgaatagctgggattacaggcgcatgccaccaagcccagctaattttttttgtattt

SEQ ID NO: 175 - DNA, 1300 bp, Homo sapiens
  source 1..1300: mol_type = other DNA
ccctgaacagtcagagtttactgcccacttttgctggaggagaagctcctgaacaactagagagactgtggttcccaaagagcagcctgtaggcctgaggactgctctatgaccggcgtcagtccctgcctccctccctccgtccctccttccctccttccttcccaggccttctctgactaccagatccagcagatgacggccaactttgtggatcagtttggcttcaatgatgaggagtttgcagaccatgacaacaacatcaagtgagtccacttggatgccccctgcacgaggcacgactccccctcctcgctgctgaagtcccatgggggcagctcccttagtccttgccgggagataacaggtgtttccagttgcatgagggtgctgaggcccccagtgagaaccaggggaggagcactgaggcctcagatgagcaccgggggaggagccctgaggccccagatgagcaccaggggaggagcactgaggccccagatgagcaccgggggaggagcgttgaagccccagatgagcaccagaggaggagagctgaggccccagatgagccccgggggaggagctctgaggccccagacgagcaccgggggaggagcgccgaggccccagatgagcaccgggggaggagcgccgaggccccagatgagcagtgggggaggagccccgaggcccccagatgagcagtgggcggggcagggagcgccgaggccatcccccttgctcttgcagcgccccatttgacaggatcgcggagatcaacttcaacatcgacactgacgaggacagtgtgagcgagcggggctgtgcggggtcatgcaggcaccctgttcccaggcagctcaggccgcgcccatggctcggtctgtggtgggcctgtgcggtggggctgggagaggcccctctgtggagctaggaacagtcgcttttcttgaccctccccatcatgccctccagcccatggcgcccacatcctgaactaagcccctctgggagccctgtggggagagcgcctcctgtctcccccagaccctctggaaactgaccttggcgttttactctgcagcccagcgcggctctgaggcctgctgcagcgaccgcatccagcactttgatgagaacgaggacatctcggaggacagcgacacttgctgtgctgcccaggtgaaggccagagccaggtgcggggcctgcccatccccccaaagcctctgccgaggaggtgcagcccccagaacacccgtcagatgcccagacgccctgctgtttgttatgccgg

SEQ ID NO: 176 - DNA, 110 bp, Homo sapiens
  source 1..110: mol_type = other DNA
tttgggccacgaggcaagttcaaagcgggagacttttgttttataaaatgatggtgagcagctccggttttatgtcaaacatcagggtttcgtgcaggatataaacattt

SEQ ID NO: 177 - DNA, 1500 bp, Homo sapiens
  source 1..1500: mol_type = other DNA
attgccgtactttgcttccctttgtatgtatttcttgtatgctgccgagtcactgatggctagctctgtctggcaagtaattcaaaaatgctgtttatgtagaaaggaaaggtagggactttaccacactctgtcattaaagggagcaattgaagaacaaaggaactgagtaaatacctatatattgccttttgtgttgcgaaacactgtagcacaaacacatttgtgttcagccaaatgttttacttccttttgtaataacgcatatagtaggttgtctccacatatgtacaagaatccatattttatttaaacgtatatagtcaattgttcatatttataggctgcaaacatttctcaatctcaaagacttttacatatccactcccacacagctatttgttattattttaaaagttcttaaattaaaaaaaaaaataaaatatactaatatctctgttggttgattttattaagcaacttaggatttcaacacagtttaaatcatattgatgactcagatcctggcaggtcttacaattcctgtgaaatgagagcacagctaataaaaatattaagcaattacttttattaaaatcatagggtttttttcattatcacatagaaatgattgatctatacagattggtctcactcatgtgtcttttgggctgcttgggagcttcatgtagaagtggaaagtcccctttgctcttccttcgaccaaggtggggaaaatgaaggcatagaatacaatctagggctattaaagaattgctggcattacttctctctatcacgtgtgagcctggctgcctgcttcctgaggtaggggatccaggatgagactgtgccggagcctgtttccacaactgcatttggagatccgtcttattgattagcgggggaaaggggtggggatcaggagtgtgaggtgaggggaggaccaactgacgactggctcaatgaagcacaagacattttcttccggaaagatgtcaaacaactgagaaacagccagagaggaagtagaaaggtggaaaaatgaggagaccctggaagaaatgaaggcatttcctatgagacagccttggggcttttttcttttctttctttttttttgcttccatcatctgacctgcaaaggctagagtgacagcgtcatgcaaatgctgcagtccagcaggtctgggagagggtggatgctagactgtgagttaatgttaatgatgagcgcagtgaaaataccagccgctgccaccccctgctcacagaagcgctctgagtcagcatcagatgctttgcctcgcctctcgctgtgtatctgtatgcctgtgtgcgcgcgcgtgctcgctcgggcatccgtgtctagccgaggggagggggtggcgtgtgagtgcgtggagggtaaaagccagtcagtcagtgagaagcaaaggtacgttggagagcaactaaaatctgactgatttccatctttggagcatcagatgtattccc

SEQ ID NO: 178 - DNA, 200 bp, Homo sapiens
  source 1..200: mol_type = other DNA
gcagcctcctcctgaaaaatgtaagccatttccactttgtaaagctacgtttatattccaccacgatacgatggaaaagaaaacccaaggcaatttaatatacgggttgggaagaaagttttgctgatggaactacattagcctccactccagcaaagcaaacaaggaaccacactaaagaaatgtactgaatcttttaa

SEQ ID NO: 179 - DNA, 800 bp, Homo sapiens
  source 1..800: mol_type = other DNA
tgcctgagcgcagagcggctgctgctgctgtgatccaggaccagggcgcaccggctcagcctctcacttgtcagaggccggggaagagaagcaaagcgcaacggtgtggtccaagccggggcttctgcttcgcctctaggacatacacgggaccccctaacttcagtcccccaaacgcgcaccctcgaagtcttgaactccagccccgcacatccacgcgcggcacaggcgcggcaggcggcaggtcccggccgaaggcgatgcgcgcagggggtcgggcagctgggctcgggcggcgggagtagggcccggcagggaggcagggaggctgcagagtcagagtcgcgggctgcgccctgggcagaggccgccctcgctccacgcaacacctgctgctgccaccgcgccgcgatgagccgcgtggtctcgctgctgctgggcgccgcgctgctctgcggccacggagccttctgccgccgcgtggtcagcggtgagtcaggggccgtctccccgaagaacgagcggggagaggggaccacggggcgcggcgggcagcctgttctcgggcggaggctctccggggcgttggaaacctgcatggtgtaaggacccgggaggaggcggggagaaattgattgtgctgttctcctccctctcttctctaacacacacgcagaaaagtttaaatttttgtgaagcgcttgcttacgtagctgcggagcgagcctctgcttcattacgagcggcatagcctttttcaggagtgatttccactttctttgtgagagagttgaccacac

SEQ ID NO: 180 - DNA, 600 bp, Homo sapiens
  source 1..600: mol_type = other DNA
ttcaatttacactcgcacacgcgggtacgtgggtgttcggggtagggcactgatctggggaaggtctcccccccgcgacccaactcatctttgcacatttgcagtcctccctcggtgcactcctggcggggatctggccagtgcagcgcactgggaccgagggcagagcccgcggagtgaggccaggagagacttcaggcctctaaggacacagctgaggctaaggctgagttgaacgcagcccctcccgcggctcgtcccctctccagtgtctctcccgtaaggtgccgctcccaacagcaatgggtcgagatgtagaggaaacactctgtacgttatttttccgcccaccctttagcgcctgaggagacagacagtgtagactttagggtacaattgcttcccctctgtcgcggcggggtggggagcgtgggaaggggacagccgcgcaaggggccagcctgctccaggtttgagcgagagagggagaaggaggtccacggagagacaagaatctccctcctcccacgcccaaaaggaataagctgcggggcacaccgcccgcctccagatcccccattcacgttgagccggggcgcg

SEQ ID NO: 181 - DNA, 600 bp, Homo sapiens
  source 1..600: mol_type = other DNA
tcattatccgattgattttcctggtatcacatcacttaagtttaagtagctcttatgttacttagtaatgactgcaaaacacgagttgtgatgcgggcaatttggatacaacaaaaagaagccattaagtttgttcgttagttaacaggtgaaagctctcaagttattaaggataaaaatgctagtatatatatatatggtttggaactatactgcggattttggatcatatccgccatggataagggaggaatactataatcaggtttgttttaaattccatgtctaatgacttcgttatctagatcacctgtagagctgtttttattgtaggagttttccttggttttaatcttttgatttgtttttcatgttaatactgaaatttttaaaaattgcatattgtacttcctatatgaaaattttactatgtatttttatttttattttccttttcctttaggaagaattagtttgttccctgacagagttagagtaagggcaaattacttgtctctataaacaactcagatgttttgagccggtgttgtaggggttatctttttctggttttgcattttattataggacatagtgctt

SEQ ID NO: 182 - DNA, 140 bp, Homo sapiens
  source 1..140: mol_type = other DNA
agaaagaagaaatccggtaaaaggatgtgttattgagtttgcagttggtgtttgatcttgcacagattttctcaggggccttaagaccggtgccttggaactgccatctgggcatagacagaagggagcatttatacgcc

SEQ ID NO: 183 - DNA, 900 bp, Homo sapiens
  source 1..900: mol_type = other DNA
cgaagatggcggaggtgcaggtcctggtgctcgatggtcgaggccatctcctggtccgcctggcggccatcgtggctaaacaggtactgctgggccggaaagtggtggtcgtacgctgcgaaggcatcaacatttctggcaatttctacagaaacaagttgaagtacctgggtttcctccgcaagcggatgaacacccacctttcccgaggtccctaccacttccgggccccccagccgcatcttctggcggaccgtgcgaggtatgccgccccacaagaccaagcgaggccaggcttctctggaccgcctcaaggtgtttgaccgcatcccaccgccctacgacaagaaaaagcggatggtgttcctgctccctcaaggttgtgcgtctgaagcctacaagaaagtttgcctatctggggcgcctggctcacgaggttggctggaagtaccaggcagtgacagccaccctggaggagaagaggaaagagaaagccaagatccactaccggaagaagaaacagctcatgaggctacggaaacaggccgagaagaacatggagaagaaaattgacaaatacacagaggtcctcaagacccacagactcctggtctgagcccaataaagactgttaattcctcatgcgtggcctgcccttcctccatcgtcgccctggaatgtacgggacccaggggcagcagcagtccaggcgccacaggcagcctcggacacaggaagctgggagcaaggaaagggtcttagtcactgcctcccgaagttgcttgaaagcactcggagaactgtgcaggtgtcatttatctatgaccaataggaagagcaaccagttactattagtgaaagggagccagaagactgattggagggccctatcttgtgagc

SEQ ID NO: 184 - DNA, 1400 bp, Homo sapiens
  source 1..1400: mol_type = other DNA
gcctgaagaccatttcttcctctcttagggacctgctggtctccagctgattcggtccaggaggaaaaacctcccacttgctcctctcgggctccctgcaaggagagagtagagacactcctgccacccagttgcaagaagtcgccacttccccctccagccgactgaaagttcgggcgacgtctgggccgtcatttgaaggcgtttccttttctttaagaacaaaggttggagcccaagccttgcggcgcggtgcaggaaagtacacggcgtgtgttgagagaaaaaaaatacacacacgcaatgacccacgagaaagggaaaggggaaaacaccaactacccgggcgctgggctttttcgacttttcctttaaaaagaaaaaagtttttcaagctgtaggttccaagaacaggcaggaggggggagaagggggggggggttgcagaaaaggcgcctggtcggttatgagtcacaagtgagttataaaagggtcgcacgttcgcaggcgcgggcttcctgtgcgcggccgagcccgggcccagcgccgcctgcagcctcgggaagggagcggatagcggagccccgagccgcccgcagagcaagcgcggggaaccaaggagacgctcctggcactgcaggtacgccgacttcagtctcgcgctcccgcccgcctttcctctcttgaacgtggcagggacgccgggggacttcggtgcgagggtcaccgccgggttaactggcgaggcaaggcgggggcagcgcgcacgtggccgtggagcccggcctggtcccgcgcgcgcctgcgggtgccccctggggactcagtggtgtcgcctcgcccgggaccagagattgcgctggatggattcccgcgggcagaggcagggggaaggaggggtgttcgaaacctaatacttgagcttctttgcaaagtttccttggatggttggggacgtacctgtataatggccctggaccagcttccctgttggagtggccagagaagtgtgtaaaacacactagaggggcagggtggaaaaagagactgccttcaaaacttgtatcttttcgatttcattttgaaaaataactacaaatctattttaattttacaaagttagactcatagcattttagatatcaatgtcttcatttaacagaagtgaagatggagcaaacgctcaatcagcgtctgtatttattcgctcctgttgtgccagggtgcgtttttgccgagcggttgcctttctttactcacaaaacccccttgatgtctgtcctccacgttttacgagggagagccggatcttttgaagtttgtatcatctaaagcaggtatattgggatgactatggatagaatttaacctgaaaacactgaagttgacagctgacaaag

SEQ ID NO: 185 - DNA, 200 bp, Homo sapiens
  source 1..200: mol_type = other DNA
cataacaagagtcattctaatgtgattataaaggacccgaagctttgcttttaaaattcaatacttaggtagaaagaaaatgataactttttccctttgatttttattcactatttttataacactagcagccctgagacaccggattggaaatatctatgcctcttgatgttacctgggcaccactgcatcacagtcct

SEQ ID NO: 186 - DNA, 400 bp, Homo sapiens
  source 1..400: mol_type = other DNA
aatagtaattgccaacagtcaagatatgtactaccaccaaattccgtgttatttgtgatcaaaagatatacacagatacttgaaaactgatttctacgttgcatatgggaaaaatacctcatttttctcagctgtccattatttttgagatattatgtgcagtgatagtaagaacaagcagatttggaacacatcagcaataattttttcaatcagagtcctgccaaaatgaaagaatttgacagtatccggcaccctgtactcatgcttggcttctgtagaaactgtggcttgcaaaagggcagctgggtactgtgttttggtacctcattctttaaacgtataatgggaatctggttggttcaggaaaacccttgcctacttattattactctgtttt

SEQ ID NO: 187 - DNA, 250 bp, Homo sapiens
  source 1..250: mol_type = other DNA
ggcccatacttaatgtatttttaaacgttttaacatttactaatatagaaccttctattgcctatttccttctggtttattccctttccttctgtcattgaagaaatggttctagtggtagaaatactccacgattgagaagaatgtgggaagaaaggagggctggtgggtaagaattgctcatgatgtctccctctgaattctgtgctctcacaatgacactccaatgtgtggtttgacgcctggaaga

SEQ ID NO: 188 - DNA, 250 bp, Homo sapiens
  source 1..250: mol_type = other DNA
tgcttcaaccggaaatgtggttgaattacccttacagtgaacctgatcagtggtaacaggagatgctagaacaggaaaagacaagtttcccctttcctccctatcccatcaattactttgaggtgtattttttctttgcaacccctccagagaagtcggcaatgtttaacgagcatgcctgccaagtggcttgccttatacctcattatgaagtgatactcagggccactaacacatcgcacagcattgc

SEQ ID NO: 189 - DNA, 500 bp, Homo sapiens
  source 1..500: mol_type = other DNA
tatgattccctcgatttccctcaatcttaaccattgtggatcacagcaggagggccagaaagtgagcttcagcctggcaccgggacctcagcctctcccttaaactttccctaatcctcggagctagtgttactcaagtgactccacagtgttgcccgatcccttcagacatggccttgatgatctccaaaactcatgctacctttgccagcctaaagcatccactctgtgccccaaaacgtgaatgtcaaatacccttcaaggcagaaggctatttctatttttgtttgtttctgtttaaggcaacaatcaccaacatttggtacacatgagccatcctgtgaaacatcaaggcgcttcgttggcagcaagtcaacttcggtttcagaagaaagctgcactatttcctgaggttagaggtttaaaccaaaacaagacaaccacattttaaccccaaatctgccgactgagggtaaccatgatccttccttcacagcacc

SEQ ID NO: 190 - DNA, 150 bp, Homo sapiens
  source 1..150: mol_type = other DNA
tactaaatcaacccaaacccgagaacccggtcatggagaaataaatgatagtaatctatgctgttcatctgttccatcactcactcactctcttgctgaacaagaaagggccacccatgtagcaaaccacatgtaaagagccgggaagac

SEQ ID NO: 191 - DNA, 300 bp, Homo sapiens
  source 1..300: mol_type = other DNA
tattattttgttcaaagtagacgggtatactaacatctgtgggcaagtttaccacacgccacttaaaacaggctaacagggtcatatgccaaaacgttcaggtttgcatttttgaaaagctcagagatctgacagatgtgttccggccgcgatttaacatgcggctccagtgagaaggaagcagatatgacaaatggttcacttatttcagaactaaaaccccagaggagcagcctgagccaaaaagggaagtgatcaatggaaaagacggtcgaatctgctcacaggcaaggcaagggg

SEQ ID NO: 192 - DNA, 300 bp, Homo sapiens
  source 1..300: mol_type = other DNA
aagacctggagtttccattacaccgaattggcacttaataactgttgtcggagcatttcttaagccacattttcgtaaagtggctttaaaattgctctgccagtaggcaggttgctaagatggtcagagacaaacttctgaacgactcttgtaaaatatacagaaatattttcagaacttttatcagtaaaattacaaaacgtgttgcaaggaaggtgcttgtgataacactgtccccagaaccttagtgaagttaccaactggtggaaaattttctcttgcactcggcttaaaaatcat

SEQ ID NO: 193 - DNA, 400 bp, Homo sapiens
  source 1..400: mol_type = other DNA
gcaggggtgactggtcctctctctctgcacctcgcaggatttctctggaagatctgagcccgagcgtcgtgctgcacatgaccgagaagctgggttacaagaacagcgacgtgatcaacactgtgctctccaaccgcgcctgccacatcctggccatctacttcctcttaaacaagaaactggagcgctatttgtcaggggtaagtgcgaccctagaggcgatcgtctctgctgtctgtggaaaaaagagctcctacacccaaagtgcttctcagttgctgacacttgatccaagctgctaatttaatctaatgtgaggctgagttttctgaatgtgggataaagtcgtagctaaacctgcttctcagggagtgccttttatctgcaatgtttttcaaat

SEQ ID NO: 194 - DNA, 1100 bp, Homo sapiens
  source 1..1100: mol_type = other DNA
aagtaacgggatcaaattaattattattttggtggccgcctctcttctccaccccaagccaggcaagactcaccctcggccctgcccgccccagcatttcaaatggaatacctaggtggcccagggggacccctgacccctatatcctgtttctttctgcctgctttgctacttttctccttgataaaaggagagagtgagagataattaacaaaaaacatggccccaggacaatgaaacaactggccttggccggccagaaatgtatcctggttttctaggtgaactttctcccatcaatctttcctttaacctctctgttagtggaagcaataggaacacccctcccctcccctgagcaaatgctttcttttgactggaaacaaaacaggggctcggcgaaggctgaggtgaaatctgggtggcatgggcgccgcacaatggggccgctgttccccggcccgggcttgtgttttacaacaggggaggggcgggcgtgaatggtctgatgattggaacaatccccccgattcaggcctacaaacgcatcttctgttccacaccgaggggacagaaaggagaaaagtgacaaagaacgcggggcggggggaattaaaacaaaatgcgctcgactaaaaaatctctcatatcctgcatattccagaaagcggctctatggagagagccttcaggaggcctcagccatatctgaatggctttctctggcctctgatttattgatgaagctgaagcgacttgctggagaaaggcctggagccttctttgtctccgagatgaagtacaataggccacagggcggagatctcttgtgatgctctcgggtcctgcctttctcttgccctctcctccctgcaaataccagcagcggtgacaaacgattggtggtgtgcctgggagagccggtgacaagactgggccacttgaggtctccttaagagggtattatggccagggcgacgtttgtgctgtgaagatggcacactccattttgtcaatggctctcatcggcccagataatcgccccctgcctgcctgtcaggggcgcagccggccgattcatggcgccctcggagaaagta

SEQ ID NO: 195 - DNA, 10000 bp, Homo sapiens
  source 1..10000: mol_type = other DNA
gtctttcccgcccccttgtctaaactcaaaaccgagtccgggcgcgccttgcagggcgcccgagctctgcagcggcgttgcgggctgaacccatccggcacaaactgcgggccactggcccctcacacctgggagtttgcggcgctggcctgcagcccggggcccacgtggcggaagctttcccgggcgcgcgctgcgcagccccgcggggccggggagacaccgctcgggagtcctccgctcggctgcagaatctttatcagctgcactttaccgcagccctggctaggacgctaggcggtggagcgccctatccaggtgcgccgccgcaccatggatcaccgcgcccggtcccgcagtcccgccatggcctggggaggcccgaagcccggggacagtggccggcccatctccggctccgcggacccccggctcaggcgggagggcaggcgggtccctgcaggcccccagggagcccgggagcctctctctggcgtcattcagtcccggggcaacctgaagcgcggtagatattggagagggggcgtctgttggggggacctggcgtcattactgatggctagcagggaggagggaacgggttgtcacctcggcctcataaggccgtgagtgagtagtccagggcctcttcaggcatttttgaaactggattaactaggggggaaattgtagcactgaagccaccgtgactgtcttttgcgctgtgtggaaactccggtaaaactctttgggcaacagtcttatcaccagctcttcaacgtgtgcagcccttctggtcctgtccctgttctgggccccaggaatgcaaagcaggtccaggcactgtgaagaccctggcggtggaggaagaggcttcccggctgtggaggaagccagacccttacaacacaagacgagaaccagacctgcgtgggggagctctggatgctacaggggctcaaggaggggtggaggggccttcccaggccaacccctgaacggcttggacaagatgctcagatggacgggaggaacggcgtgtgggatgggggagctggaggcgggtgggtgggggggggaggatggggaaagcgctggcccacccagtgtgggaggggtagaggaaaagcccgcaggggccaggttgggaccccgtaggccgggttagagggcttggacttgatcctgacaggcgacagggagacatattgctacttattatgtgcacagtggccagatctctaaagaaaacaccatcccccacccccaccccccatatagtaaaccaggtggtccgcccagtgctcccagggaggtgatgggaaatcccactccataccctgcggtgaggggttccatgccctccacgtgtgcaactactccgggcccagggaaacactgggccccatccggtaacccccggcccagtcgggtttcccagttcacattataaccaaacggtcttgccagctagacagacagacacccctgacctgtttaccctgatcctctgctctcaggattaatcacaacttgtcgaagggggtggcttccagtggggtggaccgctctgtcaatgccagcgtgtgtctagcatctcctggggtgggggtgtggggaagggaggtgtaggatgaagccctagaagcctcaggcaattgtgatccggtgggctggatactgaagcccacccctgccttgacctcaattttcagtatcttcatctgtaaaatgggaacaacctgccttcctcctagccctaaaggggctgctgtcaagattggctgagatagctgtttgcaagctgagctcaatgaaagttcattgtgtccccctcagtcctatcccaatatcgtctcactgcaaaggtggggggcagcttaacttcaagggcacttcaaggatagccaggtggctgtcagcccagctttccaggatgggagcaggatcttgacagaagggttgactgggaggggcagttgctggtttgggcttcgttaggttgcatttttgtttgttgtcctttcatttccctggggcagcaccccttcctgcaagctccaggccttcctctggaatgctcctagagcccaacctctgctggtgcctgagcttaagccaggccagctaaggggatcctggattcacacggcctcacagtcactcagattgttagcagaagacaaaaattacaaggggagggcgtcatgtgattcttacacaccctccaaatccagcagacaccttggaagccacaggtagcttcaagaaacccattttacggatgagaacctgagatggagaaaggacaactggagatctctgagtctctgagcccacactccctacctccctgcacctccaggcactctgctggcaggatcttgggcaaatgcccacagctctctgagagtcagttttcctgtctgtaaaatgggagtcataccttcctcctatggccggtgagagactaaattaaactatgtctgtcaagacacctgaaactcctggcacaatttaggttgccttcaagtggtcacagttgtcattaggtggaagtcaacaccccaatcattgtaaaggtgcccatataccccaagatccagattacagctctcacagtttattatatacagcgaaaaaacacataacacacctttgcccacatttacatgtattttacggaccatgtttcacatcagtccgcatgcacatctgcacgtgtgtgcattcggcagtatttaccaagcacctgccaagtgccagggcctgtcctccgcacccggcgtgaactgtcctggaccagtcccgggagccgcggttctgaccagccgtgctgaccctggacgactccatgagctgttttgtgagaaagacacgccatttgtttgcagagttctgacttctgaggggtcatgtagcacatgtttggtagccaaacgctgtcattcacgaccaggagcgatggctgcaatgcctttttctttgctttgctttccggtgccgggagccttgcctcccgccgccacccctggtcagctctgcgcaagaacgtcgttctgtttggcagccaggccgagacgcagcctgaatgtgagcaggaactcggagaagggaagggagagaatcagaaagaaggcccgggagggacccgggaagcagtgggaggtctgcgccctggagccccgcgagagcccgccggtttggcacgggctcctcccgggccgcccggcggtccaacaaaggccggccccgacacgcacccggtcttttgtgggagagaaacacaaagaagagggaaaaacacggaggaggccaacagcaccaggacgcgggggccaaccaggaactcccggagccggggcccattagcctctgcaaatgagcactccattccccaggaaggggccccagctgcgcgcgctggtgggaaccgcagtgcctgggacccgcccaggtcgcccaccccgggcgccgggcgcaggacccggacaagtcctggggacgcctccaggacgcaccagggcaagcttgggcaccgggatctaatttctagttattcctgggacggggtggggaggcataggagacacaccgagaggtactcagcatccgattggcaccagggccaagggagcccaggggcgacacagacctccccgacctcccaagctactccggcgacgggaggatgttgagggaagcctgccaggtgaagaaggggccagcagcagcacagagcttccgactttgccttccaggctctagactcgcgccatgccaagacgggcccctcgactttcacccctgactcccaactccagccactggaccgagcgcgcaaagaacctgagaccgcttgctctcaccgccgcaagtcggtcgcaggacagacaccagtgggcagcaacaaaaaaagaaaccgggttccgggacacgtgccggcggctggactaacctcagcggctgcaaccaaggagcgcgcacgttgcgcctgctggtgtttattagctacactggcaggcgcacaactccgcgccccgactggtggccccacagcgcgcaccacacatggcctcgctgctgttggcggggtaggcccgaaggaggcatctacaaatgcccgagccctttctgatccccacccccccgctccctgcgtcgtccgagtgacagattctactaattgaacggttatgggtcatccttgtaaccgttggacgacataacaccacgcttcagttcttcatgttttaaatacatatttaacggatggctgcagagccagctgggaaacacgcggattgaaaaataatgctccagaaggcacgagactggggcgaaggcgagagcgggctgggcttctagcggagaccgcagagggagacatatctcagaactaggggcaataacgtgggtttctctttgtatttgtttattttgtaactttgctacttgaagaccaattatttactatgctaatttgtttgcttgtttttaaaaccgtacttgcacagtaaaagttccccaacaacggaagtaacccgacgttcctcacactccctaggagactgtgtgcgtgtgtgcccgcgcgtgcgctcacagtgtcaagtgctagcatccgagatctgcagaaacaaatgtctgaattcgaaatgtatgggtgtgagaaattcagctcggggaagagattagggactgggggagacaggtggctgcctgtactataaggaaccgccaacgccagcatctgtagtccaagcagggctgctctgtaaaggcttagcaattttttctgtaggcttgctgcacacggtctctggcttttcccatctgtaaaatgggtgaatgcatccgtacctcagctacctccgtgaggtgcttctccagttcgggcttaattcctcatcgtcaagagttttcaggtttcagagccagcctgcaatcggtaaaacatgtcccaacgcggtcgcgagtggttccatctcgctgtctggcccacagcgtggagaagccttgcccaggcctgaaacttctctttgcagttccagaaagcaggcgactgggacggaaggctctttgctaaccttttacagcggagccctgcttggactacagatgccagcgttgcccctgccccaaggcgtgtggtgatcacaaagacgacactgaaaatacttactatcatccggctcccctgctaataaatggaggggtgtttaactacaggcacgaccctgcccttgtgctagcgcggttaccgtgcggaaataactcgtccctgtacccacaccatcctcaacctaaaggagagttgtgaattctttcaaaacactcttctggagtccgtcccctccctccttgcccgccctctacccctcaagtccctgcccccagctgggggcgctaccggctgccgtcggagctgcagccacggccatctcctagacgcgcgagtagagcaccaagatagtggggactttgtgcctgggcatcgtttacatttggggcgccaaatgcccacgtgttgatgaaaccagtgagatgggaacaggcggcgggaaaccagacagaggaagagctagggaggagaccccagccccggatcctgggtcgccagggttttccgcgcgcatcccaaaaggtgcggctgcgtggggcatcaggttagtttgttagactctgcagagtctccaaaccatcccatcccccaacctgactctgtggtggccgtattttttacagaaatttgaccacgttccctttctcccttggtcccaagcgcgctcagccctccctccatcccccttgagccgcccttctcctccccctcgcctcctcgggtccctcctccagtccctccccaagaatctcccggccacgggcgcccattggttgtgcgcagggaggaggcgtgtgcccggcctggcgagtttcattgagcggaattagcccggatgacatcagcttcccagccccccggcgggcccagctcattggcgaggcagcccctccaggacacgcacattgttccccgcccccgcccccgccaccgctgccgccgtcgccgctgccaccgggctataaaaaccggccgagcccctaaaggtgcggatgcttattatagatcgacgcgacaccagcgcccggtgccaggttctcccctgaggcttttcggagcgagctcctcaaatcgcatccagagtaagtgtccccgccccacagcagccgcagcctagatcccagggacagactctcctcaactcggctgtgacccagaatgctccgatacagggggtctggatccctactctgcgggccatttctccagagcgactttgctcttctgtcctccccacactcaccgctgcatctccctcaccaaaagcgagaagtcggagcgacaacagctctttctgcccaagccccagtcagctggtgagctccccgtggtctccagatgcagcacatggactctgggccccgcgccggctctgggtgcatgtgcgtgtgcgtgtgtttgctgcgtggtgtcgatggagataaggtggatccgtttgaggaaccaaatcattagttctctatctagatctccattctccccaaagaaaggccctcacttcccactcgtttattccagcccgggggctcagttttcccacacctaactgaaagcccgaagcctctagaatgccacccgcaccccgagggtcaccaacgctccctgaaataacctgttgcatgagagcagaggggagatagagagagcttaattataggtacccgcgtgcagctaaaaggagggccagagatagtagcgagggggacgaggagccacgggccacctgtgccgggaccccgcgctgtggtactgcggtgcaggcgggagcagcttttctgtctctcactgactcactctctctctctctccctctctctctctctcattctctctcttttctcctcctctcctggaagttttcgggtccgagggaaggaggaccctgcgaaagctgcgacgactatcttcccctggggccatggactcggacgccagcctggtgtccagccgcccgtcgtcgccagagcccgatgacctttttctgccggcccggagtaagggcagcagcggcagcgccttcactgggggcaccgtgtcctcgtccaccccgagtgactgcccgccggagctgagcgccgagctgcgcggcgctatgggctctgcgggcgcgcatcctggggacaagctaggaggcagtggcttcaagtcatcctcgtccagcacctcgtcgtctacgtcgtcggcggctgcgtcgtccaccaagaaggacaagaagcaaatgacagagccggagctgcagcagctgcgtctcaagatcaacagccgcgagcgcaagcgcatgcacgacctcaacatcgccatggatggcctccgcgaggtcatgccgtacgcacacggcccttcggtgcgcaagctttccaagatcgccacgctgctgctggcgcgcaactacatcctcatgctcaccaactcgctggaggagatgaagcgactggtgagcgagatctacgggggccaccacgctggcttccacccgtcggcctgcggcggcctggcgcactccgcgcccctgcccgccgccaccgcgcacccggcagcagcagcgcacgccgcacatcaccccgcggtgcaccaccccatcctgccgcccgccgccgcagcggctgctgccgccgctgcagccgcggctgtgtccagcgcctctctgcccggatccgggctgccgtcggtcggctccatccgtccaccgcacggcctactcaagtctccgtctgctgccgcggccgccccgctggggggcgggggcggcggcagtggggcgagcgggggcttccagcactggggcggcatgccctgcccctgcagcatgtgccaggtgccgccgccgcaccaccacgtgtcggctatgggcgccggcagcctgccgcgcctcacctccgacgccaagtgagccgactggcgccggcgcgttctggcgacaggggagccaggggccgcggggaagcgaggactggcctgcgctgggctcgggagctctgtcgcgaggaggggcgcaggaccatggactgggggtggggcatggtggggattccagcatctgcgaacccaagcaatgggggcgcccacagagcagtggggagtgaggggatgttctctccgggacctgatcgagcgctgtctggctttaacctgagctggtccagtagacatcgttttatgaaaaggtaccgctgtgtgcattcctcactagaactcatccgacccccgacccccacctccgggaaaagattctaaaaacttctttccctgagagcgtggcctgacttgcagactcggcttgggcagcacttcggggggggagggggtgttatgggagggggacacattggggccttgctcctcttcctcctttcttggcgggtgggagactccgggtagccgcactgcagaagcaacagcccgaccgcgccctccagggtcgtccctggcccaaggccaggggccacaagttagttggaagccggcgttcggtatcagaagcgctgatggtcatatccaatctcaatatctgggtcaatccacaccctcttagaactgtggccgttcctccctgtctctcgttgatttgggagaatatggttttctaataaatctgtggatgttccttcttcaacagtatgagcaagtttatagacattcagagtagaaccacttgtggattggaataacccaaaactgccgatttcaggggcgggtgcattgtagttattattttaaaatagaaactaccccaccgactcatctttccttctctaagcacaaagtgatttggttattttggtacctgagaacgtaacagaattaaaaggcagttgctgtggaaacagtttgggttatttgggggttctgttggctttttaaaattttcttttttggatgtgtaaatttatcaatgatgaggtaagtgcgcaatgctaagctgtttgctcacgtgactgccagccccatcggagtctaagccggctttcctctattttggtttatttttgccacgtttaacacaaatggtaaactcctccacgtgcttcctgcgttccgtgcaagccgcctcggcgctgcctgcgttgcaaactgggctttgtagcgtctgccgtgtaacacccttcctctgatcgcaccgcccctcgcagagagtgtatcatctgttttatttttgtaaaaacaaagtgctaaataatatttattacttgtttggttgcaaaaacggaataaatgactgagtgttgagattttaaataaaatttaaagtaaagtcgggggatttccatccgtgtgccaccccgaaaaggggttcaggacgcgataccttgggaccggatttggggatcgttcccccagtttggcactagagacacacatgcattatctttcaaacatgttccgggcaaatcctccgggtctttttcacaacttgcttgtccttatttttattttctgacgcctaacccggaactgcctttctcttcagttgagtattgagctcctttataagcagacatttccttcccggagcatcggactttgggacttgcagggtgagggctgcgcctttggctgggggtctgggctctcaggagtcctctactgctcgatttttagatttttatttcctttctgctcagaggcggtctcccgtcaccaccttccccctgcgggtttccttggcttcagctgcggacctggattctgcggagccgtagcgttcccagcaaagcgcttggggagtgcttggtgcagaatctactaacccttccattccttttcagccatctccactaccctcccccagcggccacccccgccttgagctgcaaaggatcaggtgctccgcacctctggaggagcactggcagcgctttggcctctgtgctctttcct

SEQ ID NO: 196 - DNA, 500 bp, Homo sapiens
  source 1..500: mol_type = other DNA
ccggcacggcccgcatccgccaggattgaagcagctggcttggacgcgcgcagttttcctttggcgacattgcagcgtcggtgcggccacaatccgtccactggttgtgggaacggttggaggtcccccaagaaggagacacgcagagctctccagaaccgcctacatgcgcatggggcccaaacagcctcccaaggagcacccaggtccatgcacccgagcccaaaatcacagacccgctacgggcttttgcacatcagctccaaacacctgagtccacgtgcacaggctctcgcacaggggactcacgcacctgagttcgcgctcacagatccacgcacaccggtgcttgcacacgcaagggcctagaactgcaaagcagcggcctctctggaccgcctccctccggccctcctgagccctactgagccctgctgagtcctggaggccctgtgacccggtgtccttggaccgcaagcatcctggtttaccatccctac

SEQ ID NO: 197 - DNA, 1000 bp, Homo sapiens
  source 1..1000: mol_type = other DNA
ggacgcggcccgctctagaggcaagttctgggcaagggaaaccttttcgcctggtctccaatgcatttccccgagatcccacccagggctcctggggccacccccacgtgcatcccccggaacccccgagatgcgggagggagcacgagggtgtggcggctccaaaagtaggcttttgactccaggggaaatagcagactcgggtgatttgcccctcggaaaggtccagggaggctcctctgggtctcgggccgcttgcctaaaaccctaaaccccgcgacgggggctgcgagtcggactcgggctgcggtctcccaggagggagtcaagttcctttatcgagtaaggaaagttggtcccagccttgcatgcaccgagtttagccgtcagaggcagcgtcgtgggagctgctcagctaggagtttcaaccgataaaccccgagtttgaagcccgacaaaaagctgatagcaatcacagcttttgctccttgactcgatgggatcgcgggacatttgggtttccccggagcggcgcaggctgttaactgcgcagcgcggtgccctcttgaaaagaagaaacagaccaacctctgcccttccttactgaggatctaaaatgaatggaaagaggcaggggctccggggaaagggaaccccttagtcggccgggcattttacggagcctgcactttcaaggacagccacagcgtgtacgaagtgaggaattcctttccaccaagagcgctcattttagcgacaatacagaattccccttcctttgcctaagggagaaaggaaaggaaacattaccaggttcattcccagtgtttccctggagtaatgctagaatttacttttgtcataatgcaaaattaaaaaaaaaaaaaatacaacgaagcgatacgttgggcggatgctacgtgacagatttttccaaattttgttgcggggagagggagggaggagaattgaaaacggctcacaacaggaatgaaatgta

SEQ ID NO: 198 - DNA, 100 bp, Homo sapiens
  source 1..100: mol_type = other DNA
tttttaatgctcagagaagttcgtattactgattcgggaacactgagtttttcagctcctgtaaaactattttcaggtttattttcaagtacattcttta

SEQ ID NO: 199 - DNA, 400 bp, Homo sapiens
  source 1..400: mol_type = other DNA
caccctagaggcaaggacggggtctgtgtcaagaggcttcccagagaagtgaaaactctgcaggtgcagccgctgggagagcatcaagaagggcagggtggaggggcagggggcgaagggagggggtgaagcccgcaccctacccccacatgaaactgattccactaccccatctctgcaagcgtccagaggcagagaggccaacatttcggggacagcttggaggcgggagatttaggcagggctccttaaacttttatgtgcatgaaaatcaggccaatcacggggctcttgagcaaatggggacgatgattcagcaggtctgggctgaggcctcagattctgcacttctaacaagttcccaggtggtagtgatgctgccagtccaaagaccacactg

SEQ ID NO: 200 - DNA, 5000 bp, Homo sapiens
  source 1..5000: mol_type = other DNA
tgcttcagtggggtaaacttgaaccgctgagaagacaagcagggagtcggtctcgctgagatttttacctgtggttctaggaacgcagaggcatgtgagtgttcaggctttgcatagaccactaagccacttctaagaacaaggctacctgagccattttgcaaaaatatgtacgtgccgaggcttttcctccccacacctacctcaactctttctgccgacacactgcacttttcaagggaacccaagtttgggttcggcaagaattgtacgttgcacaccgtgtgtgataattccagggaatttcaatcgcatcttgtcttccttcctaagcaaattcggtgggaacctggtgtggtgtgatagaaaaagccccgagttctctgtggtagaccacatcaatttcatgtgccagtctctcagactccggcttgcctctctcaaggaagggaacaatggtttgcttggcttcactcctctctttccccccaatttccacatgggtatctggctaaaaatgagttacaggtttccttctgtgagaattgcatggactgataaagtaccatcccaggaagaaaacaaagatgctgtcttccctttcggctcacagttgccgttggggagggaacacacgctgtaaattataggcagccagaagtgaccgcattgaccactgcgagtggcccagctatggcaacaggctgagaactctgggggagagccatttgttggcagggatggtgattcttctagcatcaagctctaagatgatgaccaaacggtatcaaaagaaatgatattttgctacctctccggcttgggtgaatgatgtggacagttaacctggacaatttaaacctttatgttgatggatcacttggatgaaattaaccaggaaattgccaagatttcacttggccctctgacatcaaatctcaatattatattaccaaattagagattctaaagaaccctgagttcctttcactgaaaggaaggagtggaaaaacctttccagatgatcccttttgagtcttggtgcgagctcaggccctccctacactgcctccgtgaaagctaaccgacccttgttcctaacctagcgcaggtcagctgagtgtccatcgggcacaggagccctgggcttgtccgggagatagccagactcctgctatttcctgatgtctgcatagctcagcgtgtccctcaccatctttgccgttggccagtaaggagagccccaggggccagcactgcacactgaaacccaacctattgctcaatggaatgcttaaaaatttcctgaatctgccttcctgagttgataaaataggaaacaatacacgttctgagggggtactgaaagcagagtaaagccaggaagatcttttttttctgttattctatacaaatattgcttcctctgcttgttagcagcccagaggaaatgcagccagggagccgtttgcagcttttcaccagtggccggtgtctctgtgttaccaaccaaacgacgctgcaagactagtgactaacgcacgtctgcatgattcaacttcactaaaattccctctgctgccagtaaagaagcacttgaaaactctttaatttgaaacttgagcttggttaatgacttgttttcttctctttctctttaacttctctcttgccatctccaacacacacacacacacacacacacacacacacacacacacacacacactctctctctctctctctctctctctctctctctctcatcaagttttttaatttcagggacccggaaacatacagccccgtgcattcacaatagcatttgctgtgataaagtggccggcaagccctctgcattcccctgctcacttagctgtatgaataaataatgagtcacagatacaatttgggtgctcaagagagtttgtagccagaaaattaattattctcccatcccagcccactccatctcagctttgccaaaccatcaagatacactttgcaggcactggtcagagtgcgtgccccgacgcacacggcaatgcctttgagacattttatgttattatttttgtttgtttaagcacagccctcttttaccacgaaagatacacaagacgcacatgcacacacatactcacacactcacagctcaaccacagctttgtccatttcaagaggctggtttcaaaaatggagacaggttttccaccctggctgttcctattcataagcctgtaatctaacgacttaagctgcgagaatgcttaactcgggaaacttctctattgcccttttccagagagacctcggtatgccacaatttgcttcctttctctcttgaaagatgctggttgtctctttgcattgaggctacaaggaaaaacacagcacagccccatgctgatgattttaacctaaccaagtctgtcagtctcctgtactctctgccttatagagacagctgccttgccactttggccctgaagtccccaggctggtgcaaggctatctgagagcctccgcctcctgccccacactggcaccagccctcctggctggctctgtgcatgtgcctgctaagccccagggcaggctgcattctgggccacacagcatgccgagttaaggataactcagacacaggcattccgggcaagggacagcaaaataaaacccagggagcttcgtgcaagcttcataatctctaagcctttaaacaagaccagcacaacttactcgcacttgacaaagttctcacgcaccgactgaacactccaacagcataactaagtatttattaaaacatttctgaagagcttccatctgattagtaagtaatccaatagacttgtaatcatatgcctcagtttgaattcctctcacaaacaagacagggaactggcaggcaccgaggcatctctgcaccgaggtgaaacaagctgccatttcattacaggcaaagctgagcaaaagtagatattacaagaccagcatgtactcacctctcatgaagcactgtgggtacgaaggaaatgactcaaatatgctgtctgaagccatcgcttcctcctgaaaatgcaccctcttctgaaggcgggggactcaatgatttcttttaccttcggagcgaaaaccaagacaggtcactgtttcagcctcacccctctagccctacatctctctttcttctcccctctgctggatacctctgggactccccaagccctattaaaaaatgcacctttgtaaaaacaaatattcaaattgttaaagattaaaaaaaaaaaaaaagccagcgccgccttggctgtgggttggtgatgctcaccacgctgcgaaaccctgtggtttgcattcagtgtgattcgtcctgcctgctgaccactatgctgggttcagacttctgacactgccaggctacccaacttgtggttctgtggttgtttatgaggcccaaagaagttttcacacaacccaaattacaaatttaactgttcccctttccacagcccatctcaattggttcttgccaatcatgtgacttaagtgatgtcaatttttttttttcttttctgagcaatgcccttccttccctccacctgccctcccccaggctgtgcaagaaaatagccgagtagactttgcaagagggggggatgtagaaaaaagtgactcagtcacttattatatctcaatggtctttgctgatttagtacaactcggctcctgttgttatttgtggtttttggaactactgattattttgataaagatttcattgctgcttattcaatagtaattcaacgctggcatcaagccgctgctccgacaggatgtggatcccatcatttaaaatgctaggcatcagctccgggagagttaagtccttggtaacgtctatcatggcataagtgaaactataaaagggaaaaataaataaaaagaaatgttttggtgagagtctgacccctacaacgggctggcaactcacaggtattttaaagcctgggaaagggaaagaattttacttttgaaataaaaggactgttttaatgaaaccaaaattatgtggttttattccccctaaatggacaactttagtatgtatctctttcagtaaagagataaaatcatagtacagtcttaacacacacacacacacacacacacacacacacacacacacaaattaggaagctaaaggaaaacaaagcagagagaatttctgtatttgggacaaagcagtggttactctgcagatgtttatttgtattgtcacttgggaaagctccctgtattgcctttctctagttcaattcaaatcaataggctaatttacacctgtaggtaaaactacactttgagcacatgaggatgccacaatagaaggggaaccaggaggagacacttctcctggggctgactaatgaatattatatagcgcgtcctctaccttagaaagacatgcctgtttgaagatgctaaaaacaggataattttgtaagtgggcaaaccactgtggtcacacgtatttcattttccggccccactggctttacctgctgacaactaaaacgtcattttgttttgtagttccaagatgaagaaaggcttattttcctgatttactaccttattcatttggctctgctctgcctacatccgccatagcactctgcgcacgtgaaatttcgacacatagggtcaagagaacctgtgtgatgatgggttgtaaatgccagtcctggattctaagctgcagtagccagcacaggcacttcagaaaggctgaactcccacaacactccctcggttttccctcatccacttaatttcacacacacaaagacccacaacgatagtagcttccatggcacaagtctttcaaaaggaacagacacaatttttacttactcctgttttgactaaagcaggaattgaaactcaacagaccgctttctcttacacttgtgagaagttagctggccacatgt

SEQ ID NO: 201 - DNA, 500 bp, Homo sapiens
  source 1..500: mol_type = other DNA
agggaaaagagataacgaaagaaagaaagaaaaaaaaaagggccggcaatttcatgtacatttgttttggcattcgctgaattctagagatgaaaacaatctcctgcttttaattcagtccacgtgcaacaaagttgtacgttgggagatctggcttttaataagaacgattaacaagcgtttttgatcacaggaagttgagaagagtcgctgcttctaagaatacaataaacattgactagcagttagacggtccatctttctctatcagccgtttagcagcctctactttgatttggggcaaatgcgagatgggaccaggagagagctccccacacccccaccaccacgtgggcagtggttctgttccagagcgccttccttcctgtccagggaggcaggctgctgaggccgtttctgggcaagaggccattgtcgggatatttgctttagatagcttgcagctgggctgagtgggtgtttcattcagactcaacaca

SEQ ID NO: 202 - DNA, 700 bp, Homo sapiens
  source 1..700: mol_type = other DNA
agcctggcgcacccgccctaatttgagtcagggaccctaggcgcctgcagctccggttcgggttgagtgcctcctgtcaggatgtgaagctgctgtcccccccgggggcctccagcactgctgaggactcagcagtcagcctctcctcccacttgggctcatttacagagagcatctccaggaatcagtcatggggaaaggggaaacgcggagtgacaacacaacacgtagaaagttctctgccgccttggtcaggcttgtcagcctcacagcccatcctgctcctgcgggaggaaaagtgagcagaactcagcccggagatgagccgcaggccggcagcccctgcctctgccctgcttgttgtgactgcaatgcaaggctctctgtaggtgcgggggattcgggttaaatgggtctccagtggtccagcgctcccagcaaaggccgaccacaagaattagcgggctagttatttaccataaccatatacaaaaccacaagcatcagcgttccctcaaatacatccgagacgctgtatatctctttattaaagcctgtcagggtttgttattgcacagcttggccttgaaccccaactaaaccaggctgcttgagcaaagaaccaagcaatgcaagcattcaggcaggaccattataaccctgaggccaaaggcagaagcagggagaggagacgtcttcc

SEQ ID NO: 203 - DNA, 500 bp, Homo sapiens
  source 1..500: mol_type = other DNA
agaccagcctcggtcttcggcctgcgggttctgcaaagtcaggctagctggctctccgcctgctccgcaccccggcgaggttccggtggggaggggtagggatggttcagccccgccccgctagggcggggcctgcgcctgcgcgctcagcggccgggcgtgtaacccacgggtgcgcgcccacgaccgccagactcgagcagtctctggaacacgctgcggggctcccgggcctgagccaggtctgttctccacgcaggtgttccgcgcgccccgttcagccatgtcgtccggcatccatgtagcgctggtgactggaggcaacaagggcatcggcttggccatcgtgcgcgacctgtgccggctgttctcgggggacgtggtgctcacggcgcgggacgtgacgcggggccaggcggccgtacagcagctgcaggcggagggcctgagcccgcgcttccaccagctggacatcgacgatctgcagagcatccgcgccc

SEQ ID NO: 204 - DNA, 1500 bp, Homo sapiens
  source 1..1500: mol_type = other DNA
aaacgtttaaaatatatttctaaacagaatgggccaattcagtcacagtaactgttgatctccatagcagagcaacccacaaagacagaactgatttttttcccataatcaggggtgaaaaatatacaacttgtttctgaaccaaaaccacaatttctgcagtttaaaatgtttcactgctaatatggccctggtagaaattatgtagtttcttttcttctttaaaaaaaaaaaaaattaaaaaaatttcctaagacactaaatgctccatctggaatgtagattctgatcacaaagcagctcagttaacctaaaaaataaaaaattcccatcacctgtctcagtagggcctgagagtagtgtggggaaccccagctttggtatggagagtcatggccccttgaaccagatagagaccttgaatagccatagctggtgcttctctcaggataaactctgatgtaggaagtatcaccctcatgagagtggaatttggtcatccagttgacgcagggcatattccatgtcttcttttctgagacacccaaccatccccactccatccttctgcacatccgtgtaacaggcatccccagcttctcgcgtgtgatccttcaggtcctgccagctgcctgatggaagaagtccatttcttccataaatagcatcctctgcatctcgagggtcctcgaagcgcacggaggcgaagggcacaaggccgtaccggctcttgagctcgatctcgcggatgcggctgtacttgtagaacaggtcctgcggctccttctcgcgcacgtgggtcggaaggtttccccacgtagatgcacccgtcgccctcccagccgcgctcgtgtccgcccagccggacaaccgcaccgcccgacgctgctggccagccgcagcccgcatccgcccgtatcgccgccgctgccgcctcagcacggctgcccccgcagcgtctgttttgttttattctaacagggtctctctctgtcgcccaggctggagtgcagtggcgtgatcttggctccctgcaacctctgcctcccgggttcaagcgattcacctgcctcagcctcccaagtagtgggcattataggtgccagctaaccatggccggctaatttttttttttttttttttttttttttgagacagagtcttgctctgtcacccaggctggagtgcagtggcgcgatctcggctccctgcaacctccgcctcctgggttcaagcgattctcctgcctcagcctcctgagtagctgggattacagctatgtacagcgatgtctgcaaagatagggatttaacagcactcatatcttcatgttcataaaaaagtcctacacgcgtgatgtacgtctagatctttccttttgtcacaggatatagcacggtagttacggatatagtctccgcagtgcctgggtttgactcagcttccccacgtactgtcctgcgcatattttgtgtctcagtttcctcatctttaaggtag

SEQ ID NO: 205 - DNA, 17000 bp, Homo sapiens
  source 1..17000: mol_type = other DNA
cacgcgccccggcctggctggaggggccaacccagcggggcccgcctgcccgccggcctttctgtaactttctctctttaaacttccaatgaatgaacgtgcctcttcttacggatttgtttagattagggaatagattcctcgctgatagcgttgctttgcaaataagacctcctatattattcaaaccaaacgagtttgtgtctttaaaggactatagcagccccattctatgttaagggttggctattacaattattatatgcttagggaaaaaatgtaagccccgtagtttgtgcttttcttgatgtacagaaaggtttatcttaggtggataggttttgttttgtttcttaaatgggatttttttggttcgtgtctttgaagggctgtttcgcgacgtcattaatgaactaatcggttttcagatttcaagacggtgtgtaattgatgtaaccactgaggaatttcagtgcacaccagactaagactcttccagcgcaggggattccagatgcttcttgggccctctggaagccatggggatgtttccagaccgaaaggagggctttgctggggagcagatgtgctgcctctccccgacccaggattttgaggccatgtttccgttaatctggaccgagagccctctgggagagggaggcaggtcgtagggggcgggggtgagggggagcgagatgaggtcgtcgctggacgctgggctcccttgtcgttgtccttttccccagaatccatggtcaggcctagggagccacccctgggtgctcgagatgagtccccaccctcactgaaggtcggtcactggatgtttgtgtgcatcgtaaggggcccaccgaagtcccgaagccttctcagggaccagcgagaaagaggagcaggcttgggagacagggaaggaaaatgcaggggaaagggctcacccctcgaccccaggtaaaattagaaggaacgtgtggcaacccaggtgcagctttggtcgctcgctcaaggactttgctagtcactaccattaattaattaatcactatcattaactaccaaggacaccgtttttattcccctaaaagcgtcaccttgaggggaatggagaattgggcagcagctatgcaaatcctgggacaggagacactgcctgaggaccctctctcactcccaatcccagaacccgaagttatccccgacaaccaagtccaagcacatgaaccaagacgatcagcttcaggcagctccttacccccacaagcggcccaggaggtgggcattatcccccacccctgggatttctccatccctccctcttctctcctgcgggagagagagctgtggtcacccagttgggcgcgatggctctggactaatggggtctctagacccagggcacaaaggccaatctgccaggggttactgcatgtaatgagataatcagacatgttgaccaacctaaaagaaaagactctcccagggagtaactcccagtgaaataatttattaaaaaaagcaaaaaagagacataaatttctctctactacttgaggaaacagcaaacagaacgaattagggtcttggcctctgcaggaataaattatttccgacttggtctggatacctgtaattatttgtaagctgtgggtagtaatactgtaattgtcccccggtcctttctggaagtagcaatgaccccaaggacaattggtgacgtctccacagggtttacacatggaaaggagtgaaaaatcgaggaattctttcagatagcccagaccaaaaatcctctcagccatgaaaaggtcatatatgtgatgctgggccaagcggacttttctggagtaaccatatcataactgattgcggatgtagacaagagcgtataaaccaaataggcttgaatcaacgcagtcctggattttctgttgcctctgcttgctggggcagtggaagttcttaaactccacttcagaggttggaaattcttccccctcccccacctccttagtgacaaggtctctgatctcctgctgccactgcaatagcctctcccatcccgcggggaacggccggagttcttcccttgatctctcccgagtcggcttccgctggggatggatcgcaggtaggcgccggcgcggcctggggaagaacagttgcggagcatctgaagcggaaaatccaagcagatgtgaggcgatccgggcccgcctcgttcctcttggggcctgaatttcttccagataagtttcctaatggaacatttctaagaggtggggtacgaggcggcttgctcgcacgcgcagtgggacagactgcgggtggggacgtactgagaggtccggacctcaatgcgtccgacccgtctccacaccgcccttttccagcccccagtctcctttcattccctactcttcaggctcctttggggccagtgggtgaaccgccatttagaacggtgcctcggactcgggggtcgtgcgctccatctctgcctcccccctggggcccgcgaggctggtccgggctttctgagctgggcgttcggctttaggcccaatacctggaccaggaatttcttctccccgcgccagaagggaaagacataggaggtgtcccaatctgcggtcaccgccgatgctcctgaccactctagtgagcacctgcccggtacttttccattccaacagagcttccagcttcatactaactatcccacatacggcctgtgggtattagctctaagtgtccttttccgagggcccgaggctccccctccagcagggagagctccgggacggcccccaccaagggttgggtttcttccttcacaattccacagaggcatccctgtccttcctacctgggaaacctcgaggtgcggtgcccgtgtacttctggtactttgcgtggtgccatcagggaccccagagccacagctgcgtgtgtgtgtggatgtgtgtgtgtgtgtgcgcgcgcgcgcgtgtacggcgaaaggatgtgcttgggggagccgagtacacaacgtctgcttgggcagctgctgggcaggcgttgggcctggaggtatctcacacccacgtatcttccagtcttcaaacacggcattgctctgcctcccgtagcgcgcttcgaacctgcctcgcggacacgtgaacagaggctgtccctgggaagataagtgcgctttcccgtaaaatccgggaaatttgccttgaggaaagtttccgttcttgttacttgtcgggtttctcccacttccacttagccatgtttctgcgatctgggtaatccctttcaagcccaggaggaattctcccgggtccataattgagggtcggaagccgtgggggtgagaaacgcattaaatcctcccgaagcccaggaggtgccagagcgggctcagggggccgcctgcggaagctgcggcaggggctgggtccgtagcctctaaccccttggagctccttctcccagaggcccggagccggcagctgtcagcgcagccaggagcgggatcctgggcgcggaggtgggtccgactcgccaggcttgggcattggagacccgcgccgctagcccatggccctctgctcaagccgctgcaacaggaaagcgctcctggatccgaaaccccaaaggaaagcgctgttactctgtgcgtccggctcgcgtggcgtcgcggtttcggagcaccaagcctgcgagccctggccacgatgtggactccgcaaggggctagggacaggcagggggagagcccgggtttgcgcacaccttccagcccctggagggagcctgctcggcttcgaacgccttcgaacttttgaccttcaaaggagtccctggaaaaggtcaggagcgcctgctgcaggcacggttgccgaaggccaggccttcctggcgcaggggagggccaggggagggaagcggatactcagtcgctgtccgacggcgagttttcggagcagcaggctcatgatcccgggccagtggcgagagcagtgacaccgagaacccaaatctccgcgcccccatccgcggcccggtgtcctcccggcccctgctgacctccaggtcacgcaccccactgctccacggctctgcagcctgtggcacacggccgagagtccccacatgatctcgacgccaaggtaaggaattgccctgcgtcctctgagcctgtctctggcctggggggccgggaaagctgcactcctggaagaggtggggttatgtgaccgccgctgcaggggtgcgcggaggactcctgggccgcacacccatttccaggctgcgggagccggacaggggagggcagaggggggacaaaaggactctttaggtccaaaatgaccctgaaggagagtccagaatgcccagtggccgcgtctgcaacggagtcttctttctccaattgccttctgccccatcaccatgggccccacctgcgccacctgcgcccaccctgtgaccctggctcagcgaccttggcccttaatcgcccaacgccgattcctcaaaattccggctgcgctgaatcgggctgcttttgccgccgccccggcagttgggccctgtttccgccggcgccctgggagaggcctcaccactcggctgggctccctggcccctcccttcccctggcctgagcgcccctgcggcctcccgctcctcctgagaaggcgacaatctctttgcaccttagtgtttcgaggacagaaagggcagaagggtcacttcggagccactcgcgccgttttcacgtgtgtgtgtaatggggggaggggggctcccggctttccccttttcagctcttggacctgcaacaccgggagggcgaggacgcgggaccagcgcaccctcggaaggctcgatcctccccggcagggcgcctggccaacgagtcgcgccgcctcctctcggccgcgcctgctggtgaccttcccgagagccacaggggcggcctcggcacccctccttccctcgccctccctgccgcccatcctagctccggggtccggcgaccggcgctcaggagcgggtccccgcggcgcgccgtgtgcactcaccgcgacttccccgaacccgggagcgcgcgggtctctcccgggagagtccctggaggcagcgacgcggaggcgcgcctgtgactccagggccgcggcggggtcggaggcaagattcgccgcccccgcccccgccgcggtccctcccccctcccgctcccccctccgggacccaggcggccagtgctccgcccgaaggcgggtctgccataaacaaacgcggctcggccgcacgtggacagcggaggtgctgcgcctagccacacatcgcgggctccggcgctgcgtctccaggcacagggagccgccaggaagggcaggagagcgcgcccgggccagggcccggccccagccgcctgcgactcgctcccctccgctgggctcccgctccatggctccgcggccaccgccgcccctgtcgccctccggtccggaggggccttgccgcagccggttcgagcactcgacgaaggagtaagcagcgcctccgcctccgcgccggccgcccccaccccccaggaaggccgaggcaggagaggcaggagggaggaaacaggagcgagcaggaacggggctccggttgctgcaggacggtccagcccggaggaggctgcgctccgggcagcggcgggcggcgccgccgggttgctcggagctcaggcccggcggctgcggggaggcgtctcggaaccccgggaggccccccgcacctgcccgcggcccactccgcggactcacctggctcccggctcccccttccccatccccgccgccgcagcccgagcggggctccgcgggcctggagcacggccgggtctaatatgcccggagccgaggcgcgatgaaggagaagtccaagaatgcggccaagaccaggagggagaaggaaaatggcgagttttacgagcttgccaagctgctcccgctgccgtcggccatcacttcgcagctggacaaagcgtccatcatccgcctcaccacgagctacctgaagatgcgcgccgtcttccccgaaggtgaggcctcaggtgggcggccggggacgctggggagcccggcggccccggcccaggcgggaagcgcaagccagcccgcccagaggggttgccgcggcctggcgtccagagctggggcgtctgagggaggttgcgtgagggtcttcggcttcggcgctggcttggggcgaggggccagggccttggcggcccaggcgaccaaaccctctcctggtccagggctgggtgagggcgaattacgaattgttccaggggcaggcagtcccccagcccgcacggccagcgagttctttctggttttgttctttctccctttcctccttccttccttcgccagtgcattctggtttggtttggatttttttctctctttctttcctttctttctttctttctctttctttttctttctttcttcctctttctttcattctccccttccttccttccttggccccctctctccctccctccttccttccttcctttgccaatgcattggtttgttttctttccttttctgctttccttcctttctttggaagttcactctggttttgctttctttctttccccatcccttcctttctttatccctccttcccttcctccttttctttctacgattccctttatttttccttcattcctccctctttttgtctcttctggaggaggtgaaggagggtcagcttcaggcgctgcgagtcagcggggatcacggtgaggcccaagcactgcaggctgaggccacagagcgaacacttgtgctgagccgggccctctcgtgaggctggggtgcgggaagtccgggcaggagagacccgcccccgccgttgctgagctgagacccggctgaaagagaggggtccgattaattcgaaaatggcagacagagctgagcgctgccgttcttttcaggattgaaaatgtgccagtgggccaggggcgctgggacccgcggtgcggaagactcggaacaggaagaaatagtggcgcgctgggtgggctgccccgccgcccacgccggttgccgctggtgacagtggctgcccggccaggcacctccgagcagcaggtctgagcgtttttggcgtcccaagcgttccgggccgcgtcttccagagcctctgctcccagcggggtcgctgcggcctggcccgaaggatttgactctttgctgggaggcgcgctgctcagggttctggtgggtcctctgggcccaggagctgggagggctgcgccggcctctggagccccgggagccagtgccgaggtagggagacaacttccgccgcagggcgccggacggtcggggcagagcaggcgacaggtgtccctaggccgcagggcgcttccatagcgccatccccaccaggcactctactcgaaatcggaaagctcgaccttttgcgttcgcctctgccaagcctgttatttgtgctggccgctgggtctggagctgcgcttctcggcccctccccggtggagcgcagagggctggtctgcaagcgcggcctccagccccgcggctccccggcccaggagccaggcgcgggctgacccgggagcacccggcagcggagggggctggaagcggaccctaggcctctcctgtgccacccggccctaccgcgcggccgcggggcgctctcctctcgggcgcagcggtccttcagcccagggcaggttcctccctttcctactcggaacgtggcaaagataccccagtcccagcccctccagctgagagctgttgcccaaggtcgtcgctacttgtccgctcaatggtgaccccttggcagagaactagggatgattccactccggttgatgttttaggggaaattaaaagaacattcggttttctgagtctccttccggggaggcgtggtggtaactggtttgctgggaagagccgttccttaaccgcatgcaacaaagcaggtgtggaatccggacgagagggcactcactgccttctgccccctttggaaatagaaaaagccttcgaagcagcaatccaaagatcaaatgatttgcggtcaatgatttcaattaaaccagaaattagtaagggagggccgagaagacacggctgctcagaagctgttcgctgtttgagggatttcccggagagcctgttaaaagatgcgaagtggtgggtgtaccgctcagccacctttaaaccggctctgtgcgttctggctctggaaagcaagtctccaggcatttgggctcagaattgctgggccccgagtttgggcgggggtggtccttctgggggtcaggccttgagcagcttgcactggtggcaggtttgggagcagttgaggggcttcctgtgtgtcttttggagggggtgaccctggaagttggcactctggaagggagctgtttggccctagagttttggaaagggccctgaacctgttcggtccccctcggaaagggaagggagcagtggcttagtccctccctcctccattcgtgcaatgcctggggtaggggtagacctggagccggtggactcatatccttggaattcgtcaggacagctgctccggggccttggccctcagtcagtctggggctgaggagtagggaagctgggaacttggggcagaggaagaagatgcgtttagaaagacctccattatgcaaactggagtccatttatgcaaactggtcacccttccagtagctccaaagagtggcagtggagtggcatcttgattgatttaacctcttctcaggggacctgggtctgcgagggaggatatggctgcggggttggaataggatctgtctgagctgccagggtcagggtggtggccctagggaggttttagggccagggtggtcccgggctgtggcaggggctctcagatcgcctcgggctctcagctgcaaggtgaaaaataccatgaggaattgatctgccaagggcggtcttgtctcaaagcaagtggattgctggggtaaagaatctagagaccagcttaggactctgggaggaagaaaaaaaaaaaaagaatagcatagtcctaaggaactgcaaggatcaccagattaacccttcatacctggggaaattaaggccagacatgacacaggcctttcccaaggctctgtagcaagggcaatagcaggccagttgctgccactgcggtcctgtggggcatgttctcactccactgcacccaggaggctgccagcctctgttccttttaacatagatctcctcagttgttaagacagaaagaggaactcagaggggtccctgtgtgcaaggcagagggagaccaccagaaccagggtaagcaccccacttggtagccagttcaaggacttggggatgttttcaacatttacagcgaggtttgaggccccattgtcatgcagcgctactcggccttggtctccttatctgtaaaatgggcccattagcaatgcacagggttgctgtgatgaagggtgaggtcccacaagcaaaagctgtgcagtgaggggggaatcctaagcattgttcctatgccattcaccccttcctgtgagctccccatattccctggctcaaaggagtcttgaatggcagggatggaggactcactgcctggactttgaagacccctgctttctgggtgaccaccttttcttccctttgacagtgaactaatacattggaggtagatagtgctgggaagaggacaggagaccacggctgactttggacatgggctcgaaattgataacttgatgagtcttggagggtggttaagataagctcggggctggggcagcgctgaggtctgatggtcagccagccctccccaaagtgtggccctccgttctggagataggggctttggaaactgcaaaagcgtcctggcaggccagctctggttgctccctggccatagctgctctgactacaggcagcaggacgcaggtcggcctctgcccatcggaggtcagaggcagggcctccagcaccagactcagcagtgccactgcaaacctggcacaacaggctggtcccaggactcagctcagcagtgaagttggaaaccaaggttgagtctccccatctccctttccccaacccgaaagacccaagatgggtgtgggtgaaagagggagaaagaattgctactccagaaactgtcatttgcccacacgaaacgaggtggggttcaaggtctgaactcttccagtgcctgggtgcctttgggtttaaattcagctgcaggtgcccccatcaccacttccacctgagcacaccacgagaagccaggttatcttagaaactgtttcccggaatcaaagcgacttgatttggagagttgggtgaggagaaactcacccctatacccctcagggcgtcagagatgtgaggcaattctctacctccgctggaaaaaatgcagatttattaaaggtcgactgtttagcagaacaacgtagattttttacaacgctttccccgtctctgctttgaagcctgccaggctgcagctggggatccaggagggaaagcccgcaggcgcagaggggacaatccgggaagtggtaaaggggacacccgggcacagggcctgtgctttcgttgcaggcgaggaagtggagcgcgcgctgcagattcagcgcggggctagaggaggggacctggatccctgaaccccggggcggaaagggagcctccgggcggctgtgggtgccgcgctcctcggagccagcagctgctggggcggcgtccgaactccccaggtctgcgcacggcaatgggggcaccgggccttctgtctgtcctcagaatacgtaggatacccgcgggcgacaagccgggccaggctaggagcctccttccctgcccctccccatcggccgcgggaggctttcttggggcgtccccacgaccacccccttctcacccggtccccagtttggaaaaaggcgcaagaagcgggcttttcagggaccccggggagaacacgagggctccgacgcgggagaaggattgaagcgtgcagaggcgccccaaattgcgacaatttactgggatccttttgtggggaaaggaggcttagaggctcaagctataggctgtcctagagcaactaggcgagaacctggccccaaactccctccttacgccctggcacaggttcccggcgactggtgttcccaagggagccccctgagcctaccgcccttgcagggggtcgtgctgcggcttctgggtcataaacgccgaggtcgggggtggcggagctgtagaggctgcccgcgcagaaagctccaggatcccaatatgtgcttgcgtggagcagggagcggaagaggcagccggtcctcaccctcctctcccgccacgcacatatccttcttgacttcgaagtggtttgcaatccgaaagtgagaccttgagtcctcagatggccggcaacgcgccgaggtcacgctccccagaaacacccctctcccctcccctaccccagctccccctggggcgggtggtaattgggggaggagaggccgcaggcagggaaggggtgggaaagccagagagggaggcacaaagtgatggcagcccggcaaacactggggcttcgggctgggccgcgctcgtttaatcccacaaaaatcccattttggaggtgagaaatagaggttagaggtcgggcccttctggagatcagaccgaggagacgggcccagctggcgtcttaaagcaaggagggggagtcgggaggaggtgagacccctgcacccaggtggggctcccaaaccgttctggatttaccacactcccaggtccgattttccatggagggctggggttagggactggcaccttcttgttgttaaccgcatttgatattcacaagaaccctgtgaggagactttgtcaccgtttttagatgcctgaggttgccggaggggcagtgagagaatcgtctaacctggtgttcctaccacagtccaggccctgtgtcctgggctggacccacagcccctgccaccacccagaggaaggcgcgaagctggctgcctcctttacgggtctcccttaggtgccctcatgaagggggacggccacctcacagtgcaggaactatctccccgtttgctcccaaatagtcttcttggtgtggtgctgtctatggtctgtgacctgcatctggagttacccccaggaccagcttcggaagaggagggatcgcttggaggccgtgcagtgtgaggaacggcaggcagggtgtgggaccaacatgcacacactcgcaggtgctggggccagggaggaatgaggcgctggctccctttccctccatttctccctgggggtcccagcaacctggccatccctgacttccaacagcacagcgtccccacaggtcctgcagtgctctgcaggggtgcagggagctcccctccccccagccgcaacctcaccttcctcacccccacccctccggcaggaaaccacaggctgggttggggacccctggtgctccaagagagcagtgagtgctgggagccgctaaccccgaggcgcctagcacagactcttctcaccccttatttctgaaataaagcccttccttaggtccagatgaggaccacgtgctcagtgcctcactttcgtgggagtgtatatcactttacagtatcaagacaattttctttcgttacaaatctttatttagtctctgcgtttagaccaaagtagatttttatgggctgagtgaaaaaacctcgcccgcattggtttctgatggaacagctggcagcgccacggccccgggtggggtggcctagaggcaggggtgcttgggaggaacatctagcacccgaccacctccaccaggtgggaaagggacgtttgcaccaaatctccgccggcaaagcagaggctttggggaattacagaaaaactataatgatctaaaagagaacaagttatcttgaactgtgcgggtatttgaatcatacagaaaattgtcctgtgtgcccaatgcacttttgcatgtagagccagggccttcgaggaagctttcaggagatcccgggcagcggagtctggtctggagtttcatttccgtaggtgcagatttctccccaagtcttcccgccatgggctttgcaagaagccagggcccagaggccacgctcaccgttaacactgcacagggcaaaggtggctccaggacaactgcccaaccccaggaacgacccagcagcagagaaaaggacagctgccagggtgcctttgtcgctttttggaaatcagaattcctgggtccttagttaagtcttacttcaccaaatcccaggaccttcacattttggttcttgccattgctaacagttgtaaatgctgccgccacgaggcctgggaggaaggacccgctggtgagagcacagggagtgctgctgtgatcacggtggtgatgcggggtgagcgcgatttcccgggattaaaaagccaccgctgcccccgtggtggaggctgggggcccccgaataatgagctgtgattgtattcccgggatcgtgtatgtggaaattagccacctcctcagccaggataagcccctaattccttgagcccaggaggagaaattaaaggtcatccctttttaaattgaggaatagtggttttttttaacttttttttttttaggtttttagttgccgaatagggaagggtttgcgaagccgctgccctgggccgaggtgcattttacgcttccagaggtcgaggcctccagagaccgcgatgcccagggcgttcccggggaggctgagagacccagggtgctctgggtgactgcacggcgactcctcgggaacccactcgtggctgcccgcttggaagggctttgcggccccgggaacgatctccaggatctccacggctggtcaggttccccgtccctcgtatcccgcgctgcccgggggctcctgcctttggttcagtgctcgcggcaccaccgcactcaggacggcagtggggggctggggctggggctgggcctggcccagcgtgggttggggcgggggacgcgccagcagcgcccgcagctcgctccgcaggggtcgcagccaggggtcgggagctaggctcgtgggccgggagacgccgggcgcgttgtcctccggggaggttggggtgcaggcggtgcaccgaccctcgccatctggcgctgcagccaccagccacggcgcttagtggagggtctgcggccaggctcccggcggaaagattccggggagggctcgggggttgtcccagcccgcgctaagcgccgcagcctcgcccggctttcctgcttcctcggactgtgcaggggaagcctggggtctcgcggggcgcagcagtcaggtcgagggtgcagcaggaggggagtcctgacgggcaggtccctctttcccctggtgcgcaacactggttggtagcttttgcggaggtggtgaagaagggcaggaggcctgttgagcggaggagtccggggatccctaattatgtgacaggagaccctttccagttcggcctgtggcccatccctctctcaccgccggcagattggagtctgctctcggggagcccccaggtaaacccctcacagggagaaggtttcggattggaaggaggaccgcgctcgtggggcgcctgtgagagctgggaagcccaaggggtagcgtgtagggggttttttatgcgggaggagctgcctcctgggcggcggggactttctgtctcagcctgtctgcctttgggaaaacaaggagttgccggagaagcagggaaagaaaggagggagggaaggagggtccttgggggaatatttgcgggtcaaatcgatatccccgtttggccacgagaatggcgatttcaaagcagattagattactttgtggcatttcaaataaaacggcaatttcagggccatgagcacgtgggcgacccgcgggagctgtgggcctggcaggctcgcacaggcgcccgggctgccggccgctgcggggatttctcccccagccttttctttttaacagagggcaaaggggcgacggcgagagcacagatggcggctgcggagccggggaggcggcggggagacgcgcgggactcgtggggagggctggcagggtgcaggggttccgcgtgacctgcccggctcccaggcatcgggctgggcgctgcagtttaccgatttgctttcgtccctcgtccaggtttaggagacgcgtggggacagccgagccgcgccgggcccctggacggcgtcgccaaggagctgggatcgcacttgctgcaggtagagcggcctcgccgggggaggagcgcagccgccgcaggctcccttcccaccccgccaccccagcctccaggcgtcccttccccaggagcgccaggcagatccagaggctgccgggggctggggatggggtggtccccactgcggagggatggacgcttagcatgtcggatgcggcctgcggccaaccctaccctaaccctacgtctgcccccacaccccgccgaaggccccaggactccccaggccacctgagacctacgccaggggcgcctcccgagcgtggtcaagtgctttccaatctcacttccctcagcaggttccacccagcgcttgctctgtgccaggcgccagggctggagcagcagaaatgattgggctgctctgagctctgaagcattcggccgctgtgtgtgtgcaaggggcgcaaggacggagagacagcatcaataatacaatattaacaggagcacttgtccagagcttactgcaagccacattcagttccggaccttattgacttccccctcccatctagagtggattctggtttttcaatttgttttgttttgttttttgtttgtttgtttgtttttgagacggagtctcactctgtggcccaggctagagtgcaatggcgcgatctcggctcactccaacctccgcctcccgggttcaagcgattctcccgcttcagcctcccgagtagccaggattgcaggcacccgccatcatgcctggctaatttttgtagagacagggtttcacccaggctggtctcgatctcctgacctccgatgatccgcccacctcagccttccaaagtgttgggattacaggcgtgagccaacgcgtcctgccttgattctgtttttaactccattttttagaggaggaaattgaggcacagagaggttaaataacatgtctaaggtcacacagcaaggggtggagcggagttagcccactggcctagctctagagcccacccggataaccagaacttggtgaggcctccgggctcttgcttggtttggagccaggtgcttagcgccccgagcccggggccattcaccctgcaggagctgcacgcgcccctgacctcggcttttccctggcagcagaggggctttgcgggtcggccgggtagccctgagcacagctcgccacttccaggtgggctgttggcgctggctggggacacatcccgatctttcaaatgccctttacagagcctcatcaacgacccgattcattcccccctcctgtcatttgtctctgccatcgaaaaatgcctaccgagagctgctctgcatttccgccctctattttgtgttttactttaaaataataataaaaaaaatgttggctgcaggacgccatgacttaggtcagcgagtcagccgctagctctgcatttccaaaaagcagatcttttcacaactctcttgccccaagtgccctggtgtggtttattttttaaaatgcatgcctgcggaagagaagacccggggaatattcgaaaccccgagcttttacaacataaagcgcatggtgtggccgcggcgagtaatggcgct

SEQ ID NO: 206 - DNA, 1500 bp, Homo sapiens
  source 1..1500: mol_type = other DNA
caaatcacttgaactcaagttcaagaccagcctgggcaacatggtgaaaccacatctctacaaaagtaaagaaaattagccaggcatggtgctgtgtgcctgtagttccagctactcctggggaggtcgaggctgcagtgagccgcaatcacgccacttgtactccagcctgggcgacagagcaagtccccatctcaaaaaaaaaaaaaaaaaaaaaaaaaaaaggctgggtgtggtggtcccagatactcagaggctgaaaagggaggattgcttgagcccaggagttcaaggctgcagtgagctgcgatcacatcaatgcactccatccagcctgagcaatggagtgagaccctgactatatttaaaaaaaaaaaaaataggaagaaacaactcaaccacagggctagtatgttactcggttataaaatgataaagccctaaacagagaattagcccgtttccagaagaggccaagaacagatgatacagctgaactgaactcctgcctgtacagctcgttttctacaagattccagacctggaagatgatggcatccagcccccattgaagcacctcgaacaagaaaaacgccgagtccgaagagccaggccttgaacacacgattcctgtctataaataactccccctggggaataaaaagcaggatccaaggcaggaaacccgagccgtggaatctggtaagttcttaggaaacccactcacgggcctgagtcccccgtggaagcggcgacttcggcacctggacacccgagtccccagagccccgggcggccgcgcgtccctacctgcaggcctgataccggccgcggagcgctcctggccccgctcccgccaggctccgggaccgctgaaacgcacccaggggggtgaaggcgtagtcgccaaggacagcgcagatggcagcggaggcatgggagccggaacctaccgtggcaaagggccaggtcgggacgcccctcggcgcagccccaaatcctgcccgcgccccagccccgctcaggccgcgcccctgccacctctggccacacgggctgagacgtctggctcctgcacagcgcacttcccgctgcccttctccactggctgctcaggccctgcctcgccagcacggcatccgcgggggatccctacctgtcctttagggcttgcctcataggtcaaacgtcacctcccagggaggtatggcctgccccctggccaggtgggccccttccacgctcgcctgcaacaccacccacccaccttgataactgcttgtaaaggttgtactgctttcccccttgagactgcaaaccttcaagggcaggaaatgggtctgttttcctggcaaaataatgaagttggcttaaggttttgctgaataaaatgagtgacagacaaaagtagccaaatttggcactcctgatgggttatttgatgaaggaggtgcaatgtatgggcttaactagttattctggatttctttccccatgtta

SEQ ID NO: 207 - DNA, 200 bp, Homo sapiens
  source 1..200: mol_type = other DNA
caaggccggtgcacgcggacccgaggattcggtagatgtccccgaagacccgctgccgctctaaggcggtggaagcgagattctccggaaacccagggaatccgatgctcgcacaggaccaaagcccgaggccgcggggaccacagagggacggagaagccgggactcctcacatcccacatccggcaggggaagcccag

SEQ ID NO: 208 - DNA, 2000 bp, Homo sapiens
  source 1..2000: mol_type = other DNA
ctgataataaagttttaccattttataatttaaaaatgtaaatatggagttgggcatggtggttgggaggctgagaccagaagatcgcttgagcccaggggtttgagaccagcctgggcaacatgcagaaaccctgtctctacaaataaaaaattagccaagcgtggtagcacgcacctgtaatcccagctactcgggaggctgaggcaggagaatcgcttgagcctgggaggtggaggctgcagtgagctgagactgtaccactgcactccagcctgggtgacagagtgaggctctgtctcaaaaaaacaaaacacaaaaaaacaaacaaaaaaaagcaaatatatgtaaaaataggaagtgcggtttcccaaaatgaggtctgtaaacaactgatctagaaaatgttctggaaaaagtaaaaaaggatcaggatctgaggtcaactgacctctccctgcgctctggacaggcaaacaggcaaggttccctctgaggccgtagcggcttctcgtgggcgagtccctgttcgcaggtgacgtgtggaccacgctcttccgaagcgtctggcctgtgtgctctcggggaggggacgcaggtcagcccacctagccgatggctaacaagtcagtttgttttctgaacggaagcttaaacctagaaaagtaactgggttggggtgggggtgtagccacatgcagtaaaagcactgcctgtctgtataacaacgacctgatgaaaaaaggaacgcgtgaaatggggagtgttagggcgtcacaaactccagtgtggttgaaatgaaagcagaaagcaaatggcaagctggcttccccttccagcttttcacaaccctgccttgctcatggtcagccccaagcacgggcggaagaaaggactggaggggagggaaaggggtggggagcgagggtaccagaggcgtgggaggacggggacaaaggggcagcaagggaccggcggaaaggaaagtcggcgttagctggattggaaacagtccagacagaacgatgggctctgctgcctccgggtggggcaccaagcggggagcggggccacgaggcaggggacagtgaagcaccatgcagcgcccaccagccggcagcgcccaccagcctgcgctgcgctgcacatggtacccgcggccccagctggccagtgtgtggcggagatgagaccctcgtgaagagactaagcggccacagcagggggaagggttgctcacataaccccatactgctcacactacgaggttaactgccgtgagatctgcctgcagccagcagaaacccgttctaggaaaacgttgcccagtgacttcagtgagtgccactgacccgggcgcctccgccccggcgtccggcagcagcaccgattgcgcaggaggcaccttgcaaacaacctttcctgatccgcgctgcagttcccaggccggttgcagccgtttcacagagactgcgcacacaaagcgtctccgtgccctgccattcacctttcgacacagccgcaacccctcttttcagtgttaaaacctggcgccaaaaggaacatgcgatgtgacgtgttacctctgcgcatgcgccgggcattcccagcgccccgaacctgatgaacgcgcggtggggaccccaggcttccgtgctttcgttttcctggaagctacgtgtcctcagtctacatattgttacctggaaaataaagttttctccttttttcttcctttgttaacaggcagaaggtgtaggctgcaggtttcgggcctaagagagggcatggctggcgacacggagtagactcctagatgacataacggaggcgagtctgcaccggggactcggcattaggaggaggcagaggaaaagcccaccaccgtggccgagggagatctagcaagcagcttgcagggggtgaagtgtgtgcaaagcaggctgagacctgtccagtatcgaaacacgccgcggtggtcaagcaggctttaccatgct

SEQ ID NO: 209 - DNA, 700 bp, Homo sapiens
  source 1..700: mol_type = other DNA
tgaggctcaaaacaggtgtctgtgagcttcacaggcggtaaggccgtgtctacatggccgggacatgcatcccggggctgcccctgccgtgctgcccgagtgcacgggggatgaggacctgacaaggccattgatcttgcgggagcttcctgaactactccagcgtgaaaatcttccagaaggattctccacagggcaatgaggcaagaaatttacagcttagcctgattaatgggccaggcagttaagagttctttgccaagctatgagcataatttatagtcatcacggcaggaggaaaggccacataactcacatccttaaagggcccttagaacaagagacacgccggatcattgaaaacgtctccactcctggcgccaaaagagatcggcacgtttctgggtattctggtcaaagaacagggagtctggattaatatacacggcagaaaaaagcgaagaaaagacacacaggtcatatatttctgactgatattccgtttgttgttttcggagggacttggtatttatttaaccacattctcacttgacacgccccctccccacaccttgtaaatgccttcctctttagccgagtcatttttcatcacatagaattgaaatgttgccaggaaggcggtttatgagattgtagaaatggcactagagaaagcagtgtgaaaagaggcctagaacgt

SEQ ID NO: 210 - DNA, 600 bp, Homo sapiens
  source 1..600: mol_type = other DNA
tctctacatgctatctactaaaaacttaggcaaggaaatgcatcagaccaaacaccccacagcacagagaaccgaccggccattgctttccaatctccgcaaacctaaccattgctggaagaaatcttactcacagtgcacagacagtaggtattttattgaagataaacatatagtggaacaaaccaaattacccccatttgagttacgtgagcactcagttctcagcgtggatgtcccacaaatcaagtcaacatttgcgtcccattaccagcagccacttgccgagtatctcttcgcttccactgggactgcctggcatccctgatgctaaggagccactgaagagcctccaaatgtctgacattcacaaacgcatcttttgctttgacccgacccttcaacctctccgagtctgctgccttttctcagacacacatccaggcaccgttagggatagttagagaatctgaaaattcagaagcgctccgaaaagcctttccaaaagtaatccacagcactcaacagtgaatttagaaaccccaatttttttctgagtttgaagtttttaagccttgcggatggttggagtaggaaaaa

SEQ ID NO: 211 - DNA, 1100 bp, Homo sapiens
  source 1..1100: mol_type = other DNA
tcagacaagctctgtgcagtcggaattttttaaagatgcactgtcacttgaggaagacaggtgatcttcctgcggcacaaatagaagcaaagagatttctcttcttctctgtagagcaacacaattgataaatggccgataatctccaccaaattggcagcagtaggctgcccgaaggcagcaggcatattcgtctttgtgaattgttttactatgatgctgtcacatttccaggaataagacggttaaaatgatatattgttgtggtttggcatttgcagctttgctctgacttccctggtaactgccaacatctgcaaattattatgtgcttaaaaaaaaaatcaaccgccaccgcaggctgcccccacggtccctggctgggccaggcctcctgccaggccacagggcagagttcttggaccaggaggcagcagggtcaaaacccaggttgcctaggaagcccccaaagacagttatggatagagctgggagcccgaaacacatgcggcagtctctcagtttccaggtaccggttctcacatcatccatgcatgtgtttgaggaaaaacaaaaaaaaattgatggttgccaaaaacaaaaatgcttccatatcaaagtttatcagtgtcaatgtcaagagacttctggttcgtagactcattttggcttgaggccaccagaagtgaactctggtttctaaatgcagaagcagaggcactggccgatcatggaagatgcagggaactgttcaagaggcccaagcctggtgctcagaaacttggcaggatcaagcatctcgcccaggaattcatcccctgcttgtctaagccggctggctctcgtgactgactcggaacaacagagcagatgtttgcgtgggaggcaagcctcacccaacatctgtcctgcggcgggaaggcctgggtgttcacagatagagctggagttccccggtgggtggcacagacaattagctggggctgcctcacatgtaatctaattacaggggaaacaggctcaaacaccgggtgataagcagcgcaactgtttcgggtgactctgtaatttttcctccattaattttctccataacgcac

SEQ ID NO: 212 - DNA, 250 bp, Homo sapiens
  source 1..250: mol_type = other DNA
gttgcctgggatatgcttatatcaaaaacttacgtgtcacttacctagcatttgcatttcactgggcctcctaaattctgtgtggtaaccgactgccaccggacatgctgtttacttctctatcctcacgcagccagttgccacattcaacataacactgcaaatattgccggtggatcctgacttcctcgtggaccctactgtgtcgggaaaaacaaacaaacgaaccctggaaggaaacaccatgagt

SEQ ID NO: 213 - DNA, 600 bp, Homo sapiens
  source 1..600: mol_type = other DNA
tcataaatatttccaaatgtattcctatttgtctctacagagtctaacagacataaatagcgaattgaaggttctgtcttaaaacccagcagaaagaaaaacaatgaccagaaaaaaaaaacaattgtctttggcttcccaagaacagcatcggatttcaactggaaccacagatggtccgttgatagaagcgactactttttagctctggaggacgacaaaaggaaccagcttcttcctgtgggtgtcacagcgaggtcgcctggccacatcaggtaccagagcgagcgccctcacctgataggccctgtacaacctcagccacagcactgtcaggaggaacacgcggaactagcaacctaggagggtaaaggcggagttgggagggaacacgaggcaggcaggtcggctggctgctgagctacaggctgcactcctaggacgtctacgtgtaattgagaaaaataagacaaaaataacttactgtgcaggcaattaattctggttggcatagcgatcctcttaagttaaagggaatgagcatgagatgaagagaagtaagaggcagaaagaattatgcaagagcaacatcagagtgga

SEQ ID NO: 214 - DNA, 2000 bp, Homo sapiens
  source 1..2000: mol_type = other DNA
acgccgagccgcctctgcaggggaaaccgaagcagatgtggtgagataatacatccaaccctgagtgctactctaacctgccagaggcggagggttctcagtgagatgaaagcattacagatgcgttagatctaagggaggggcctgcagatgcgcagctggcagagaaaccagggaggggctgaactgtcagtcgcgaccaccagggatctgaatcagttcaccgacagccttggggacattcaccttgggctccacaacctgtcagaaatgcccccaagcccaaaggcgtcgagagaatggccaggttgtttcagattgacacatatcctaatgtacaagtcagcccacacaccccacgtgcactgagcgtctcttgttgttcaccccaaataaactctgccggaactggggcgggactcgcaggggcggagaaggggggagacgggcagagggcagaagtggatggtgagaagagccaatggaggggccccgtgagagtgagcaaggctgcacccctaaccgacgtcctggggctactgtacaaacaaagaaccacaggctgggaggctgaacaacagacctgcactctctcgcagctcggaggctgcaggtctgaaatcgaggggctgacagcgctggtttcctctggaggctgcgagggagaaaccgtcccctgcctctcccaggctctggggtgagcccttcctggcatcccgggctcattgtagatggatcactccaatctccatggcttctcagggcttccctccatgcacctcaaatctctctctccttccttttgtaaggatgccagtcattggatttaggttcaccttaaatccaggatgatctcatctaaattacatctgcaaaaagaccctttttccaagtaagttgacattcacaggtacctggggttaggattggacatatcttttgcaggggtgcagggggctgccactgagcccgctgcacagggtgacctgggccaagggcccttcactttcacttcctcattggcaagctgccctgtgtttggactgggtcgaggctgtcaaccttgctgcccctcggagtcccccctggtgtcccccaaacagattctaagctgctttcctggggctggaggccaggcattgggattttttaaagagcttcccagcaggtgagcagcctttcatgggtatcaggagaccttcctggcaaatgtggtgaaggtccttcctcctgagcgatgccttagacccaggagcccagggaggctgctcacctgatcgttaggacaggagcagtggaaacctctggcctcagaccccctggaggaatccctccctctaagactctgggactggtgcacgcaaggagctatcgtgaacattgctcccaactggccgcttgcttgtcccccggctccccttggccccagtggcggctttgcctgaattagagggcgtgagagccacctgtgtctcagcactgcaattaaagcaggaagccctttcggaagcagccgtgtgcaccagcctcccatgggtggagcagagcaaaccacccacttctgccctctgcccttcttcccttttctcgacaccctgcggccccccagtttcagcagagtttatttggggtgaaaaacaagagatgctcagcgcctgtgggatgtgtgggctgactcgtacattaggatgtgtgtcaatctgaaataacctggccgttatatggatgccttggggcttggggggtttctggcagtctgtcgagcccgaggtgaatgtccccaaggctgctggtgaatcagatccctggcgttctccgttggcagttcagcccaacagtttctctgccggccgtgcctctgcaggtccctcctctgatctgattggattaatatttgaatcaatagactgagtcaagcagaatgtgggtgggcctcatgcaatcagctgaagccctgaaaagagcaaaagggctgccccttcccccgaggaggagagaac

SEQ ID NO: 215 - DNA, 700 bp, Homo sapiens
  source 1..700: mol_type = other DNA
cacatttcagagctgaggtgctggtgcgggcaggtctcctgagctggggggtcagctgtgtggccagtgatggtgacgcctcaggccgtgcatggccggggaggcggccctgcctctgcactcttttgactccatgactactggtgtcttcggacgccagagtcgggggagcaaccatggggcaccgcccctgcctggggaggcagcacgaggcctgagcccagcttacagggggacatccacccccgctgagagccccaccttcacggcgaggatctgtagaagaagacatttgatattactcggcaaaaaaaacaagaaacgaaaacacaaaaagagctcctctgaagaagaaaaggtatttgcgctgtggtccacctagaaataatgttgttggcacaactagagcattcctcagtcattcaggagcactccctgccggtgcgtccacatgtcccaaccccgatagatgaggcgctgttcgcccgtggaggggtcaggttgtcgtgaccttatctttacccttaggccgtccatcccggggcctggggtttcctgcgccagtcacggtgggctgtgtaggtggccatgtgttcggtctttccccaggaggtacgtaccatgtgctgggaggcctggaggctgagccgccccccgcgcctatgagttgcaccctcacagcggcggccaaacctcctgc

SEQ ID NO: 216 - DNA, 200 bp, Homo sapiens
  source 1..200: mol_type = other DNA
caggcttgagcggtgactgggagaccccgggaatggaaatggcgctcaaatgctggtgtggtgtccgcaggggaacggcccgcgggtgtgtggagtctgcgcccctgtggcttcagctgcgtcgggggactgcgggaatcttccagactccagtttaaatcagagaggtgtgtccacgaaaagagtcaaactaaaacatt

SEQ ID NO: 217 - DNA, 1300 bp, Homo sapiens
  source 1..1300: mol_type = other DNA
aacgagacagtgcaaaaagccgctgcctggtgacctggcatgcagactcggccctcccacttgcacggtgatccactgaagacaacagctgcctctgtactcacgctcccccacactcccctccttcctgccctggtttctccatccctagatgccatcccatgccccaaaccatccgccaagcacaataacctcgcccccacccaccccatgaggtcactcgagttgacaaccagataacagtttttgttttgttttgttttgttttgttttgtttgtttttgagacggggtctcgctctgttgcccaggctggagtgcaatgacgttatctcggctcaccacaacctccgcctcccgggttcaagagattcttctgcctcagctgcctgagtagctgggactacaggcgcgtgccaccattctcagctaacttttgtatttttagtagagacagggtttcattatattggccaggctggtctcgaactcctgacctcttgatccgcccacctcagcctctcaaagtgcagggattacaggcgtgagccaccgcgcccaatagcaatttgatgacccatcccctccactgctgggaaaaggctgggcaccgcccacactccatgcagctctctttccctggctcggaatcgctgcaggcgccacagaccagacgcgcactgttccccactcctgcttatcggccgcgcggcatccccttgtcgcagcactccagcatccatgcagccgcgcggcaccccgtcttcggagcactccagaatccatgcagagcgcagcaccccacatccagagcgctccagaatccatgaagcacgcggcaccccctcgtcagagtgctccagaatccatgaagtgcgcagcaccccttaatcggagcgctctagaacccgtgcagcgagcagcaccccacacccggagcgctccagaatccatgaagccagcagcaccccacacccggagtgctccagaatccacgcagcacgtggcatctcctcgtcatagcgttctagaatccatgcagcgagcagtaccccacaccgggagcgctccagaatccacgcagcgtctggcacatctttatcagagcgctccagagtccatgcagccacagtcctccaacggaccctgagattgtttctgcaaaaggccatgccttcataaatctgaaaatttggaaaacatccttctacttatatccttacaacccaccattcaagctgtagaagcctttctggaaccccaagcagaaggatatccaaaatgtaaaaacggtggggcct

SEQ ID NO: 218 - DNA, 1000 bp, Homo sapiens
  source 1..1000: mol_type = other DNA
atagtgcgactgttccgaagtctttatcacagttactggtgatgcttttttccagatgtcctcgacgtgcacccatgaagggctccacctgagagtgccagggtcctccgtgggatggggctggagggggtgctcttgccgtcctgggctcccaagcagccataggaacaatagggtgatggggtcccagagatagaggccagtgacagcagcgctttgaacccctcacacgggcacgggccctctggcagggatgggcgtcccggtcacacggagatgggggctgctgctgcctgcaggtagaggaagggacgtgtttggcagtcctgtgacccctgggcacctcgcctcccccacggccggctctgcttgtaaacagacaagtgcacaagcgcagcccggtgaaggcacagcggtcccaggaggcatctgggctgcaccccagcgagccgcccatacacgtggagatgccggccaaggccctgcagcacacggcagaggaaggcgcgatgggagccatgctgggcccggaaggtgccgccgcccggagctgtagccatcactccagctcttcttttaagtgttcccagaaattgtgacccaccaaaatctgagagcacccgacagtaagccagaggaccttgatgtgagatcccagcacggtgtgggggcggactgtggtgggtgctgtctcggcccccaccccttccacaggtcggtgtgcacatcccacggcgcctgctaagctgcagtcttctccaaaggggtcactctccgtgggaagggagccacccgcccccgggtgatgtccccagtcagtgactgacgacagtccccagccgaggtgagggaccagctcctgcatccctcactccggggcttgcctgtgggccagggtgggggcgagcctcagcagagaccgcgtcccccttgcctgtcctgccctgcctcccctgcctcccccgcgcctctgctgagcacgcccagagggagctgcttg

SEQ ID NO: 219 - DNA, 500 bp, Homo sapiens
  source 1..500: mol_type = other DNA
cacttgaaaagcacaactcatggtgccaaagctctgacacggactccactggagctgtgggcagggggtgccaaggtaccgagttccaagccgttgttatttgagagcgtgccccccgccatgagagcaggtggggggacataaagtgacacaggatggactggccaaaggctgaggacgatcacttacctcacaggatgatgccacccccacggacaggcaaggagctctcaccttccccaggaccccagctgccaccagagctccagatggccctgggggtgtctgtaaagcctgtgaccgtccaccaggtggagaccaggctggccaggggagggagaggaagtgaccactggccctggcactggctggccggctccagcaggcccgaaggggagggaggagcctgggtgcaccagactctctcaataagcagcacccagacacttaacagatggaaagcggtggcttggaactcacttccaacgaaacaatagcac

SEQ ID NO: 220 - DNA, 1300 bp, Homo sapiens
  source 1..1300: mol_type = other DNA
agcacctcctaccccaccctccccattcctgccatccccagggtccagggagcccagattccagggaagggttgcattagctcccactcggagtcctgatgcagcagagacagacagaggccctgggagaagtgagcatgaattattaagacaagacaagggtgaggccccagagagggggtggcggaagggtcatgttcatgcagcgagagttgcttcgagcttgaaccgcgtatccaggagtcaagcagattgcaactggcgagaggccttcagaaatgccccgtgagagtcctgtgtgcagagctccatctcagcacacttcctgttcttttggttcgtcgatttttgcattttcagtcccctgtgatccattatttataacagtggagattggcctcagacactagcagtgaggaaaacaaaagcgaagctacgcagaaaaatgacaagagtgatgagcacagcagtcatgacaaatgagccctgtgcggaggcccgggatccgcgcagatgccggcgcgggggaaatgggccctgaaatcccaccgtcaggccaggcagctctgagcgtgacctggagggctgttcagacggtctgggtagccgtgtcctgcgcatgaacatcctccgtcgggagaggaattccccacggattatcagagctgctccctccaccccccgccacgtcccacgcgggccacatcaactccctctgcagcctctggccagcggctgagccctccgtgtctcccctcgttaatgcctccttcaccatcccctcctgaagtttcccccattgcatacacgcgctgaggcccacccggtatcaaggactcccattgcttgcgaaaaagattccacccctcttagaacagagaccagggccgctgtagcaaatggccataaatgccacagcttaaaacaacagaaacggattatctcgcagctctggaggatggagtccaaaatctgaatcgctgggctgaaatccaggtgtgggcagggccgcgctccctctagaggctcccccggagattcccttccttgcctcttccagctgctggtggctgccagcagtttgggaattgcggccgcatcacaccacctttctgtttgttgttgacatccccgcctcccctgcctgcggggtcttagatgtctctctccttcccactgagtttcactccacatttgaattggattaactcatgccatgttaggcaaacgtgcccctcaaatccttccacttaacagacatttattgaaggttcctgtgtgcggggcccaagagaaggga

SEQ ID NO: 221 - DNA, 200 bp, Homo sapiens
  source 1..200: mol_type = other DNA
gaatgttcaaagaaagagccctccttgccttcctcttcttccacccctgccctctgcagactggggttctgtagacccccaaagtaagtccgccacaccggaaggaagtgagttacacaggggcccacatgggaaccgctttttgtcctgtcttggtgggaaaatggccacgaccccagcccaggctctgccacgccaca

SEQ ID NO: 222 - DNA, 1600 bp, Homo sapiens
  source 1..1600: mol_type = other DNA
ccatcttcctaggcctgcgtttcccccacaccggggacttgtgctggaaagaaaagctgcgttggcagccaggagccggggaaactgtccagggaggcatcctctgcgatgaaggcggggcctcggcgtggcccgttccgcgctctgtccagccctggagaagccccaccctcaccgagctcgaaataccccctccctgagagccgagactcatggccgggaccccttggacagaagatgcggatgctaacccggcgcttccaccacagccccggcggcactggggagcgagcgcggccatcccgcgcgtaggtggtgtttctctgcaggcgccagtttcaccgcgggcgcccaggatcctcaacggttctgttgtgatgtgattcccctcttcgacttcgtcattcagcctcagtccctcagtccccaaataccgaaaggcagtcttttttttttttttttgagacggagtttcactcttgttgcccaggctggagtgcaatggtgcgatctcggttcactgcaacctccgtctccctggctcaagcgattctcccggctcagcctcccgagtagctgggattacaggcacctgccaccacgcccggctaattttttgtatttttagtagagacggggtttcaccatgttggccaggatggtctggaactcctgatctcaggtgatccacccgcctctgcctcccaaagtgctgggattacaggcgtgagccaccgcgcccggcctttttttcttttttcttttgaagttaatgaacttgaattttattttatttacagaatagcccccatgagatacttgaagacccggtgccaagcgacagtgttgaccccaggtggtcagtcctgcctggccccttccgagggatgcgccttcaccataaccatgtcacggacaggcgtgtgggcaagggggcatcgctgtatttttcacaactctttccactgaacacgacaatgacatttttcaccacccgtatgcatcaaccaaatgaaaagatgagcctgtgacattcccgtgcgtagagttacagcttttcttttcaaaacgaaccttcagtttggagccgaagcggaagcacgtggcgtctgacgtctccagggagacccgccgccctcgctgccgcctcaccgcgcttctgttttgcaggtaatcttcagcaagtactgcaactccagcgacatcatggacctgttctgcatcgccaccggcctgcctcggtgagtgcgcgctgcgggctctgcccggtgacgccacgcggcctcctcgccttttcgggatggctgggaggggcgggaagaggcgctgaagggcccgaggcaccggccttctacaaggggctcttcgaaatcaatcaatgcgcagaatcccgagggaggctcagccgccctccgggcctctctgcctccacaggtgatggctgtgtccacaaggaggaaaccgtcgggctgaattaaacagaaccgccctcctaagagtgtgggtttttctgccgggcgtggtgtctcacacctgtaatcccaacactttgagaggccgaggtgggcagatcacctgaggtcaggagttcgagaccagc

SEQ ID NO: 223 - DNA, 400 bp, Homo sapiens
  source 1..400: mol_type = other DNA
aggcagcagggttaggacttcaacatacaacttttggggggagatgtacttcagcccataacacaccacgtgggaggataacaccgatttcagagcttgcagaggaagccgccaggaactccagtgagacatcagcccccaggtgcctgtcaggcacgccgggctgtggggggcacctgggcccatctgagtaacggaggcgcatccgcacttcccccaggagtacatttttagaacccacagcgccataaaccaaagacaaggagacttcctggtgccccgtcagcttctggaggcgacgttctcggctgacagctctggcagcctcccctgtaggtgagagacaggtaaatgggactcttgcttccaaaacggaacagggtaaaaattctcaagcgtt

SEQ ID NO: 224 - DNA, 700 bp, Homo sapiens
  source 1..700: mol_type = other DNA
tgctgcacccccgctgccctccctcccgctggccggcagcaccttctccacccgggcccctctgctcacagcgctccccgcccccgtctccccgaggggcggggagccaggacatggccctgaaagcctagccctggccttgacctccccagagcgccctccccaccctccgccctctgccaaccctggcccctgccctggccccgtccttgtcctctgctgctggccttggggtcgcgccccgcagactgggctgtgcgtgggggtcctggcggcctgtgccgtcccacgcctacggggatgggcgaggtccttcttggggcttctcttacccactctccagtcacctgagggcgctgcttccctgcggccaccccaggtttctgtgcagccgaagcctctgcctctgcggccgggtgatcccaagaccccggggtccagggaggcacgggatctgctcccccggtcccaaatgcaccggctgcgccttaggagggacggcctccacccatggcgctggcgcccaggggccgctcctcggactacagcacttgctcgtcgccctgcgccctgtttagttctcatcaccagcagcctggactagggccctggtccttctggcctccttccacagcccgctgcacatctcacccacttccccgaggtgctgtcattgtttagctgggcccctcagcctccg

SEQ ID NO: 225 - DNA, 300 bp, Homo sapiens
  source 1..300: mol_type = other DNA
ttaaaggggagtggttgtatgaagagttcctcagtcaaaggtgtgcagctgggaagcccaccccacctaagagggaggtctgacaaactgtccacactgaaccactcagacctgcatcagggccccgtttcttccataagccgccaagtacagccctgagtcaactgaactcaggcctgggaggcttcccaaagctgacttgactcagctttgaactgaaatgaccgtaccatgacaaccctgatgaaaagctaaactgagcccaattattcaacagtaaaattcagttggtctcactca

SEQ ID NO: 226 - DNA, 450 bp, Homo sapiens
  source 1..450: mol_type = other DNA
tgctaccagctgcttgggcttgggcaagtcaccctagctctcagatgtcatctgtaaatgatgacaatgccaatgtggcactgttctgagagtcagacagaacgtatgtgtgcttcacatatggtgctcatgaagtgctatcattatctaaggaaaacagaaaacgaagttcagagtctctctaaacgcatgacaccagaccaacagggagtttcaaaaaataggtctgaagtaaatcaattctcctggtctcaatacactgaaaacaaactattaggggactgaccgaacccaccttaggaaccaccttacgtcaccttctgtctctactgcaaaaccctcccttaatactgttcaaatacgctgacaatccagatccatatccaatggaaccagcaatcatgcctgtgtgccagcaatgtcagggagggaagccgatctctgatgaat

SEQ ID NO: 227 - DNA, 1000 bp, Homo sapiens
  source 1..1000: mol_type = other DNA
caggtgccggccaccacacccggctaatttttgtgtttttagtggagacagggtttcgccatgttggccgggctggtctcaaactcctgacctcatgtgatccacccgcctcggccttccaaagtgctgggattacaagtgtaagccactgcgcccggccaagagtgaagttctgatagctggggtaagaaaggccgtgggaacagccggtttcagacacgctgggtctaagacgctgcgtctggcgctgctcggcatccaatgggagccgtggagaagccaggcgagtgcgtagggcggagccagcgcacaggaaataggacgtgatgaggtcaaccggctggtccaagtgtggacggaagtagaggatgcaagcaccgagccccggggcccccagcattggcggggaggagctcgcggtgcgggagaagcaggggaccgcgcatcctggagaccaggtggagccagtgcgcccggaaggggcgtggcccgctgacagccgcccaggaggccgggggaggcctggagccgagggccgcgcgtggcaatgtggagagacattttggtggagtcatggggccacagcctgattggtgagaacaggaagggaaattgcagatgggcctgggccccctggctcccgcatactccaggaccagggctgagtcatcgttcaccgtgtgtgaccagggccccgtgtggccggctgtcactcggtatccagttaccctgggcagaccactggcggcaccccccagccagaggccgcagcaacacacacgcctgcaggcgaccaggccggactgcatgccccgtgggggaactgagggcgtttcagtaacagagtgttaggggacacgggttgggtggcttggaaagggcctaaggtggggtttgttttagattggggtggtgagggcgcaggggcccggtaggattctctaacagggcagcagccactcatttagcaacaggagaggcgtccagcgtttcgtgggct

SEQ ID NO: 228 - DNA, 3100 bp, Homo sapiens
  source 1..3100: mol_type = other DNA
acccaaccacaggcctcctctctgagccacgggtgagcggtgcaggttctgctgttctggagggcctgagtcccacccagcacctcataaacagggtcctccccagggctgctgcagtaggcatcaacgccagggtgcaaaatgcctcagggagccaaggctgagccaggggagtgagaaggagcatgtggaagtgcgttttggagaggcagctgcgcaggctgtcagcaggctccggccgcttctatagacagcatgacaccaagggcagtgacctcattccacaggctgagtccagccagccagccaagcatcaccagccagacgattgaccctaacggaccaaccaacccgtaacgacccctcctaccataaccagtagccagccagcccataaccagccaacttatctataaccagccacctgaccatagccaaacaaccagccggcccaccagtagcattcagcccctcagctggccctgagggtttggagacaggtcgagggtcatgcctgtctgtccaggagacagtcacaggcccccgaaagctctgccccacttggtgtgtgggagaagaggccggcaggtgaccgaagcatctctgttctgataaccgggacccgccctgtctctgccaaccccagcagggacggcaccctctgggcagctccacatggcacgtttggatttcaggttcgatccgaccgggacaagttcgtcatcttcctcgatgtgaagcacttctccccggaggacctcaccgtgaaggtgcaggacgactttgtggagatccacggaaagcacaacgagcgccaggtgagcccaggcactgagaggtgggagaggggggcgagttgggcgcgaggacaagggggtcacggcgggcacgaccgggcctgcacacctgcaccatgccttcaaccctgggagagggacgctctccaggggaccccgaatcaggcctggcttttccccaagggaggggccgtgcccacctgagcacagccagcccctcccggtgacagaggtcaccattcccgagctaatgtggctcagggatccaggttagggtcccttcccgggctgcacccagccgtcgccagctccatccctgtcacctggatgccagggtggtcttagaaagaaccccaggaagtgggagtgccccgggtggccgcctcctagccagtgtacatcttcacatgaaccctacctgaggaagccagtccccgacggcatagctgcatccgcttggaatgctttacaggcattgacaccttcgcctcacagcagcactttggaaccagtgtcctcattattccagggcacggctggggaacaagggggtcctcagcctgctgggtcccacagctagtaccgggcaggtggacgggagcttctccccacagtcaccctgatgccccgctcttgctcggctggaggcctcggatctccgtggtgttgagggagccggggcactggagccctggtgacctgcatctcctggcggagccgggaagagctcatggactgtcacagatggacagtgccccgcgggggctggagagcagagtggggctggaaggtggaactcttagccaaagtcttggtttcttttggccagggtcctctttcaatggctggagaaggtggtgctggggggtgaacgctgacctcctcatgtgctgcccctccctcgcctgggcccggtaaagcccccacgtagccccagccagcctggaacatgcttcctgagctcccagctcttggtctttgcacccagtggaggaggaggtcagcccagggagctgagtctgcggtttagggcgtccaggggacgtggaagcatgtgggtcgtctggccacattaggtagggctgcagagacctgggctagagcagtcctgcggggtctggaaggggaagactggctgaggtgcggggcctggtctggaatgatcctgcgattttggagtgaagccatggagcgggaagagacaaccccccgcggggaatagcccggcaagtggccacgaggccaggctgaggtccagagaagcaggggcatgaatccataaatcccagggggcctggccatgggatgtgctggctgcacccggcccctgtgagagcccccgcaggctggcccccttctgcagtcagtggggctggggcagcttctctggcatggggcgaggcagccgcctgcacagtggcccccctgactgtgcgcccccaccctctccaggacgaccacggctacatttcccgtgagttccaccgccgctaccgcctgccgtccaacgtggaccagtcggccctctcttgctccctgtctgccgatggcatgctgaccttctgtggccccaagatccagactggcctggatgccacccacgccgagcgagccatccccgtgtcgcgggaggagaagcccacctcggctccctcgtcctaagcaggcattgcctcggctggctcccctgcagccctggcccatcatggggggagcaccctgagggcggggtgtctgtcttcctttgcttcccttttttcctttccaccttctcacatggaatgagggtttgagagagcagccaggagagcttagggtctcagggtgtcccagaccccgacaccggccagtggcggaagtgaccgcacctcacactcctttagatagcagcctggctcccctggggtgcaggcgcctcaactctgctgagggtccagaaggagggggtgacctccggccaggtgcctcctgacacacctgcagcctccctccgcggcgggccctgcccacacctcctggggcgcgtgaggcccgtggggccggggcttctgtgcacctgggctctcgcggcctcttctctcagaccgtcttcctccaacccctctatgtagtgccgctcttggggacatgggtcgcccatgagagcgcagcccgcggcaatcaataaacagcaggtgatacaagcaacccgccgtctgctggtgctgtctccatcaggggcgcgaggggcaggagggcggcgccgggagggaggacagcggggtctcctgctcgcgttggacccggtggcctcggaacgatgg

SEQ ID NO: 229 - DNA, 1000 bp, Homo sapiens
  source 1..1000: mol_type = other DNA
tttttgtgtttttagtagagatgggatttcaccatgttggccaggctggtctcaaactcctggcctcatgcaatcctcctgcctcagtagtagtagttgggattacaggtgtgagctgccatgcccagctgcaggtgcggaagctgggggcctcagagactgtggactcctggccggtgaggagcggcatgggccgggagagctgactcttcagcgggactgaggtggctggagcgtgaccctttcctgagggcaaacagggagggccttggagcccggcgctcaggacaggcccctgctggcccggcagcctgagcttccacacttttccagggcgtctcgagttcgcccacagagctgttgtttcaggataaaaaatgcccttgtattccacgttccagttcagaggcccgtctgttcccaagagcggaggcgtcagccgcatgagtcccaccggaagccgggttgccgggtccccgtccctgccctgcagacgacgcattccggagcccccttgggaagctgcctggctctcccaggcctggctgccttcgcacgagggctccgaggcatgctcatcctacgtgactgcccgagtgtgcacacgcctggccgtgtgtgggcgtgtgcctggggcccgagctcaggagcaaggcctgcgtggacctgttgtctgaaacaagccagtagacagctgcgtcaatgcaggcaagctgaacagggctgctttttcagcctgacaaccccaggggctgaacaggagctgggggaggagcaaggggccgttcccctgccccacagcacagcacacgaccccgccttggaacctggggcccggggtgaatcgagggtcctggagcaagaggggctgctccacaggagagcctgtcccgccacccctcagccaccagattcggggctgctggacttgttctcaaacctgcacagtgagtgacagctgctgagacggaggtctcaggcagtgcaggtgaatcagcat

SEQ ID NO: 230 - DNA, 500 bp, Homo sapiens
  source 1..500: mol_type = other DNA
tccttattttttagttctcaagccctgtagggtgttttcggtcgcagttgtttgggctgtggtcctgaccctcctgagttccagtggctctgttcaggagagctgcctggggccgggacttctgaaacacacactgagccacaggccggcccggcggcttgggttcaccgccgcctctttgtgtgtgatgtcctgggataggcccgtgcacgttcagatgacactgtacatataaataacttgtagccgagaacaggatggggcggggaggaggggagggcagaacgtaccacagcagcagaagtcactgtggatgccttcgtaagttgcatggaaggtttttaaacctagccctgccgagcagccctctcctggtccgggagaacgatggggagagagctggcgttcagctttcatcactggagccgttccttcttccggccccccgagggcctgtccatgatcacactttgtcttgtttcgggggtggcccctgtgac

SEQ ID NO: 231 - DNA, 1300 bp, Homo sapiens
  source 1..1300: mol_type = other DNA
caagcctgtggtagggaccaggtcagagtaaacaggaagacagctttcggccaggcggtgcacctcggtgccggtgagtgtgagcgtgtgtgcgtgtgcacgtgtgcagatgtgtgtggacgctcccttctccgcagcagctcctgaccccctgcaggtgaccctcagccagccccagggctgcccccactctcccctgtggacacctacctcatttggggtgaagtggggggactggggtgtgaggggtgctttggggggcacacttcgacccctctctctgcaggccaagtcctgaggctcagtttcctcctctgtgccccggcgacgtggtgcaggcctcgcgagtgacgtgagggttcatgacccaggtgtgggcagccagcccttcacgggaggccacccacctggccacagtgcctgggaatttaggtcgggcactgccgatatgtcgccttccacaaggcgggcccgggcctctgctgaccgtgcaccggtcctggggctgggtaattctgcagcagcagcgcagcccatgccggggaatttgcgggcagaggagacagtgaggcccgcgttctgtgcgggaactcccgagctcacagagcccaagaccacacggctgcatctgcttggctgactgggccaggcccacgcgtagtaacccggacgtctctctctcacagtccccttgcgtctggccagggagctgccaggctgcaccccgcggtggggatcgggagaggggcagtgtcgcccatccccggaaggctgagcctggtgcagccagggagtgagggggcgggaagccggggtgctgccctgagggtgccccgacacgctctcctggggccctgagcggctgccacgtgcgtccagggttctggccacagggtgggcaggggccctgtgctcctcactggaggcccctgaggctctggaactgagaccatccacccgccggccccctctcgccggctccggcacccctgcctactgtgacttcctgccccggactcgctctgccagcttggggcaaaccacttccctctggggttttcacttccctctttcccaagtggggaaagaccacctgtccccgacccagaaagggcccctgcccgagggcagcagcagtgccaggctggcatgtgaggcttggggcaggcccggcccccagaggcacagggcgatgctctgtgggacgctgtgtcgtttctaagtacaaggtcaggagaggagccccctgaccccggaggggaggagaggcagggcaggaaaccgccaccatctcagccca

SEQ ID NO: 232 - DNA, 200 bp, Homo sapiens
  source 1..200: mol_type = other DNA
gcccactgtgggtgtgcccgtgtgtgtggctgtgaggcgtgagtgcaggcgtgaagtgtctgggagtgggagcgggcatgagtgtgtgccacgggcctgctgttgggtccttggaggccacggttgcccctgaagggactgcaagctcttttttgatttgtagttatttgagaagtctatacaggaagaaaattaaaccg

SEQ ID NO: 233 - DNA, 1000 bp, Homo sapiens
  source 1..1000: mol_type = other DNA
agcgcccagcgcagggccgggacccagagtggactctaccgtggggctgcctcaaagaaatctcagcaaacacaggaagccagcccacccgtgcagccatggggccaggaagcccgccctttaccaagtcatttgggcattttttctctgtgctaacagcccagatggagccatagcctcaacctctgtgttctgataacaccaagctgggacgccggagccatgcaggggacagtgcccggcctgaggctgcagcctgggtctggatgcctttctaattcagggcctcctcatggcctggttccataaatggtcaaatgcagcctgacagcgcagcctcctatcagcgctgggctccgtaccgccacacagcccacataccccgttccccaggagacgcccgcaggtgggcagcgtcactcccacccgccgagcacacgctgtccccgtctcgtgtcccgaggagccggaagcagctgcttcctcccagcctgaaagctgcacctcgggctgcactcggctccccgaacccgccctccgctgccctgcaattcgccaagggagctacccttcccatataaaaatttcacctccatttccttgtagagaagaaacatttctgacagcaaggaagattctaatttgaaaagcaagtgattcatctcccggtgccaaacagcagacgcaggcgttaccagtctgggtggggcgcccgagctggggacctggggtcctctgggaggggcaagaaggcagcgatgctggcccccgcctccatctgcccatcccatctgcttccacacaccgccctgccgtagctgcttgcagcccttctctgtcagtttctccatcttttggtttggtgataaatgagagttcccatcgggtgtgccaccctctgtgtgacggggagcagagaagaccctgcgtccaagtcctcctgggggaagagcgaagatgctgggaccagccccagctgtcagggggtctccaatcccag

SEQ ID NO: 234 - DNA, 1300 bp, Homo sapiens
  source 1..1300: mol_type = other DNA
ggaacggagagccgccaggcccaaacctcccagaatttgcgcagtattctcggcctagagagcgaggagtggccttggcgaggtccctctttggctcttctggcttagccggggttttaaacttgttatctgcaaagcagaaggaaagtcagcccctgatgtaagtgtcaagtaaaataaatcggatgggtcctttcctgtttggcgaggaatgctacactaagggggactgcgttcaaatgggcagtctttgctggaaacctcgcctccgcgcgccttccctcgctcggattcaggcgcttttacgttaagggttgaatttttgtgtcaacaggcacctcgggaggtcgcctagacaactgagcggagcaactgagataacccccgctacgtgtggagtgacctagtccattaacttgccccagcacgcccgctgagtccgcaaaatataggatggcctcgggttttagatgaacccaaagctaagatttcttccctctctggaattagcaagcagcccgccctgcccaactcccctggaagcgcgcgtgctcgccaggcctcgggacgcctgcgcgggcgcccttgcactggcaccagggctccggggtaggggcgcaccgatctgcccaagcctctgcaggcactggaggaaggcgagccctccacccgctcaacaggccccagtgccggcctttccttccagtctcaactccacccgggggcccgggggctccacagttaaaaactccacgccacggagatcgcaggtaagctgctggctcaacgaggtgtgctaaatgggattaaagatcctggaccgtggccaggcgcggcggctcaagcctgtaatcccagcgatcagggaggccgccgcgggaggattgcttgagcccaggagtttgagaccagcttgggcaacatagcgagacaccgtctctacaaaaaaataacaaatagtggggcgtgatggcgcgcgcctgtagtctcagctacttgggcggtcgagatgggaggatcgatcgagtctgggaggtcgaggctgcagtgagccaggatcaccgccaagatcgcgccactgcattccagcctgggcgacagagggagaccctgtctcaaaaacaaacaaaaaatcctagaccgtttacaaacagccttccgtctcttcctggtcaagtcctaaccctggctaacctcgccgtctacagcctgaattttggcaaccgaaaggcagcgccggcgccacgtgcacacgggctgggccgctccgccagctgccagggccactgccgcgctcact

SEQ ID NO: 235 - DNA, 300 bp, Homo sapiens
  source 1..300: mol_type = other DNA
cgcacacacagcacagacgcctgcatcttcccatgcgtggtttctgctcttgcctctctgggtttttgtttcacttcggtcgagtttttggtggtgttgagcggatagccggggaagttggagtcttgtttgtggccgcctcgtgctcgtgtctgtatctaagatcctcaggctgctcctttttgggtaaggtctgttgcttctctaggaacagtgacggtggcagagcccgtggcccctctctcctgtcccagagccaagctgtttcctctccccactcccgggcaccctgcgggcaag

SEQ ID NO: 236 - DNA, 1000 bp, Homo sapiens
  source 1..1000: mol_type = other DNA
cacagcccagcttcaagcctggccgaccaggggtttggcatgaagaccccggcagggctggggctgtgctggaatccacccggaagtttcctgccccttgggctgcccaccaggtcccctttctgctctgatcaagctggacaaaacgtcgtggggccacagcacagggggccaacgcaagctgggatcgtcagacgttaggaaatcccaaggaagaagagaaaggggacacattcgggagacgtcggcacacgctcgaagcagcggacaggcacctctctgtggacaaggcagactgggcggccgagattccgcatagatgcctgcttcctccacgacctccacgtgtggctggcccagtccgggtccccctcacctcctctgtctgtcttggtggcctcacgccgtgggctgtgatgccggctacgctgcttgggtggccaagggtctgagctgcaagacgcccagcctgggtctctcccgagctctcccacgtcctgtctgctcctcctccgagctcccggttgactctcacgactgcaccagcctctcccccaggaaggcgtggaaacaacctccttctcccaggcccgctctgcctcctgcgtttcaaggcaaatccgttcctccaggagatgatgcaaccacatcctgttggagcccagagaagtgcggatgcagcccggggctctttctttcctagaaccctgcctgggagtggcttccctgaactaaggacagagactttgtcttcgttgcctctcggcctgtgggcactgagcatacagtaggtgctcagtaaatgcttgcaggccgatgcccagagccattagccctcatcatggtgagctcggcagccggtgttggggctgggctgggcctaggtgtgcgtgggggcggtgctggtctgctttgctgggagccatggacaccggaggaacagggccccatcagtgcggtcagagtgcaaactcggagcgtccttctctggaaaacgaat

SEQ ID NO: 237 - DNA, 500 bp, Homo sapiens
  source 1..500: mol_type = other DNA
gggagggggcgtggccagcaggcagctgggtggggctgagccagggcgatccgaccccgaaccggagcttttagcactttgagtccctgtactcagaggtctcctgcagccgggaatcccactgtgctgtggtccctggcagccagcacccacccccagcttctccgtcaaggttgaggacggagcactcctgcctctgattaactggacgcaggagaagcagttgctttaatccggagccttgagttgggacagataatgagtcattcaaccagattttccaaggacacactaactttggtatgatgcgtgtgtgcccctgaatccacgtggtcaggaaagcccagggaacactggcctgtgactcactgagcaggttcccttgttaccccgaggggtgatttactcctctgacagtgacacggacactgtgcgtccattccccgggcgggcagaggacactcccagatgcccacgaggggcccagcaagcactggcca

SEQ ID NO: 238 - DNA, 600 bp, Homo sapiens
  source 1..600: mol_type = other DNA
ctgcaggacctgctcgttcacagatgttctcctagaagcagaagctgtttcttgttgcaaacaaatttgctgtgtcctgtcttaggagtctcacctgaatttaccaaggatgcatctgtgcttggggatggctcggtttgaggggtctgaggagcggctcccctggatcctttcctccccaggagcccacctgccgagctgtcagcgtcagccccacatctcaagatgaggaaatggaggtcgaagccatgcacacgcaggcgtcctgctgacatgcaggccaggcgggtgcctctgtattcagcagcctcagggctgtggccagttcaggcagcagaggggcctcatcccggtgcttccctgcaggcagttgtggggccggcctgcagcaggggctcagacagggccttgggagagggagggatcacagaggtgtccagtgacaggcagggcgggcagagcccatggggccttgggctcctcactccttcggtcagtcagggtgacatctggagccacctccattaatggtgggttatgatttggttcccatgcagcccgtgccagctcgctgggaggaggacgaggacgcctgtgatc

SEQ ID NO: 239 - DNA, 5000 bp, Homo sapiens
  source 1..5000: mol_type = other DNA
aagaggaaattcccacctaataaattttggtcagaccggttgatctcaaaaccctgtctcctgataagatgttatcaatgacaatggtgcccgaaacttcattagcaattttaatttcgccttggagctgtggtcctgtgatctcgccctgcctccactggccttgtgatattctattaccctgttaagtacttgctgtctgtcacccacacctattcgcacactccttccccttttgaaactccctaataaaaacttgctggtttttgcggcttgtggggcatcacagatcctaccaacgtgtgatgtctcccccggacgcccagctttaaaatttctctcttttgtactctgtccctttatttctcaagccagtcgatgcttaggaaaatagaaaagaacctacgtgattatcggggcaggtcccccgataacccccagctgcagatcgaggcctagtgcgagcacaggtccccccagacccttcccagtgcccaccaaccggcggcctaggccaggtagaactggcagcgcctcccctgctgcaacaccaggctctggtagaaacttcagaaaacatgcaccggcaaaaccaaggaagggtggctgcgtcccgggttcttccgcgcagctgtgtgtacacgcatgcacacacccacacgcacacacccacgtgcacacccccatgcacacgcacccacttgcacgcccatgcacgcacacacgcgcgtgcacccatgcgcacgcacccatgcacacacacgcgcgcacacacccacgtgcgcacccacatgtacacacccacgtgcacacacccacgcgtacacacccacgcgcacacaccgctgtccccagccgtgcagaacgatcctccctgagtccccggctccgacccacacgcagcactcgctaaacgcttcccacgcagtcgttttgctgggttgcgcttcacccacttctcagagggggcggccgaggcagaggtgtcggggatcgagcagctccgggcctcaggggtcgccccgccaccgttttcctttcccagatgctgggacgggggcagggaggggctccccaggctgaacccgactaggtcaccctagaagcgaggcgagcttctcttctgtttttcttcggcgcccctgagcccctgacagtgcccaagctgcccatgggattggattcgccagagcctcctacgcagaccccacccagggccaaagccaaccccaagccccaccaccttggtggtgtgggatgaaaagtgagccatcgagagatggggtccccccacccccaacccctccaaggacaaaggcgggctgggaagcacccgctttcacgtccgcccctgcccggctttcctagcggaattggcgccggcatcagttgggggttgtgggatcagtgaggaatcccgtggggtcgcctccatttatcagttgtgtggggttgggcgagcacccctagccccagcccaggcgatcagggcgcgaagcccactggacgcggatttgggattaggacgggggtgacagccaggaggaccgcacctgccctccccactcctgccgctccacccctgcccccaccgcaacaccaaggtctccaccaggaagatgggggtggggaaaggacgcggggtgggggggggtgcggggagagaggacacagggtcggaagggtgaggggtagtggcagaggcggaggccgaggccacgcagctgcggggcgcagggaggggcagaggaggggcgttcagatgggaacctagtccagacccgtcggggccctcgtgtgcggctcgttatcctggaaccagagaggctggagacccttggcttgtctggagcggaaccgtagtgtccaatagagtgtgtggggctcagccctaaagctaaacattctttatttcctgatgaccatgggggcggagcgggggaaaagccctggccttatagtttagaattttataaaaggaaaggcgtggccactgacaatttgcgcttcaggagtcccagagtgaccgcctggctcggagcagggaatgagggggtccttaactctgagatttgttttctgagagacaaaggtgatgggtgaggcggctaagcctctgattctctataggtggcggtcattcatttcagaacatgaatggattcagtaaataaacatgatagaaaaatgccacaagccctaggcccattggagtggactggacagtctgttcccagtgtgtccctcagcctcggtcccccacccttcccggagccctgggggtcacacacatccctcctggctgcctagcctgtgccccccgattccccccctccccgccccgcgcgtgcacacacacacacacacacacacacacacacacacacaccacacagcacgaggcgacagagatatgagagagagcgagcgagagaggacgggagagagagggagtgcaagtgtgcgctgggggtaacccgtgcatgcatgcattgggggtaacaggctggagctcagatccctcccccagcccccagcaggggggactgcaggctcctggtctgagtggggagctgggccccctggacagaggactgggctgcggggtcaggaatgggcacacttcctaactgcaggacactctaagggctttggtcatgcacacgcagccaagagaaggtgtcgctggcacacagccttccaggagcggacttggagacctcgccaaggaccaggactccccagcactcacactcccttaggcgctgaagtccagaggacagaggttgagggcagagctcctgggagcaccagtggaagtaggagggctgggctggaaaacctcccccaacctcctattgcaaagaggctccagccagcagcctccacaccccagtgatcttttaagatgcaaatctgcgccatcatttatttcctcagtgccttctccagctcctgggatgcacactgcccgtccccaggcccagagacctgaccaccctcattcctccctcagcccaccctggggtctctccaccagctgacagccttcctgcagtcccctccccgaatgctgctccctgaggccctcctggacacctgcagggcaggcacagcccgcgggacctcacagcacttgctccgggcagagctgcagtttggccaagttgccagctccgtgtgggcaggggccctggcctgtggctgccacatcccgggtgggggcacggcctttcctggcgtggatgctgagcaaacgtagggggaaggggagtgaatgaggagagccaggtagctcaggggctgaggcctcactgagcagggtcccgcgtgaccggtccccaccgctgacggttcctggggtaacactcaggacagggagaggcaatggaaagagacgtggccgccctcgcatcctgcagctcccgcactcccagcctcccagcctcccacccagccccccagagcccaccagtgaccccgcccactgggtcctcagatggctcccacgggatctcctgccttgatctcctgtccacatggaggtgaagtgggttgctctgaatgaggggtgccgagcctagggcgcagcccactctcctgggtccgcagcatcacgcagcccggaccacaggctccttacaagaatcggaagggtccctgcaatcgcccttcgcactgaggcttcctactgtgtggtgtaaaaacacaggcttgtcctcccttgctgcccacggggctggagccgcctgaaaatcccagcccacaacttccccaaagcctggcagtcacttgaatagccaaatgagtcctagaaagcgagagacgagaggggaatgagcgccgaaaatcaaagcaggttcccctcctgacaactccagagaaggcgcatgggccccgtggcagacccgaacccccagcctcgcgaccgcctgtgacctgcgggtcaaccacccgccgcggctccacgccgtgggcacagactcagggagcaggatgagaaagctgagacggcgcagccacggcccggtgccttcacgcgcacagcgacacagccccagccagcggggcccacgctaaggcggaatcccacagaagcctacagagcgagcgcgcgcctgtgcttcccaaaacggaatggaaccaaggtgacttctacagaacgatctgaagccctggctggcccttatgctagtctcttgggagcgttccaaatgcagctcaatattacttacttgacttttatctttcctccctggttcgtggtatttataactgggtcatcttttaactatttgcaacgtagcttcaggggagagggggagggctttataaataacctgtattattattatgcaggttgattctgttccctgagctaaagggaacatgaaaatacatgtctgtgactcatgcccccccacccccactccagggtgtgctgaggagtctctcagctgccccggggtcctcgagcaggggagggagaaaggctggcgctgcgccctccatcgcgtgaagccaggggattttgctctgcgacaagctgacttggctctcgtattgtttgcagaatcacccagttccaaggcagtccctgcgggcaggtgcagctgtgcgggagcttcagtcctgtccccaacacccaggcagtaatggttccagcacggaaggtctacctacctcccactgcacagcccgagggctgtcctggaggcacagccatccgtccctgggtgggcaggcacgtttatgacccccacccccacccccaccccccacgcgagtcagcacgttccatactcgggtgatcgtgctcatcccctggtcatgtcatcgggatctgagtgccatccgagcagagagctgtggcccggtgccgggggtggacttcatctattccagggaaccaaggatgcatgatttgcaaacaaaaccagaagcgcaagccatctcctcgcctcccctgatagccgtgctgcggagcctgagtgctggag

SEQ ID NO: 240 - DNA, 600 bp, Homo sapiens
  source 1..600: mol_type = other DNA
caggaaccacgggacctgctgcctagcggccctgttccacccttggccgctcgcaaaatgtttaggcttcataaggtttgcccagggtcacaaatttaactcacagcaaacaatgaaatcagcgcatgattttcgagccctcgtggtcaccctcccttcctcctgccctttcctgcatgggcagcagcagggtgaggagctgctctccccaggcccaggctggagtccctcagacgacctgccggccagggtacccccctgcccccacacagcgcctgacagagccccccacactgggggaacgtggggacccaagcaggggcagcggcctcaccgggcaggcggcgacctgcatcatggcgtccagcccaccctcgggtgcatccaggtttccggaaatcagctgcttcccgacctcggtctgaaactggttggagttgttggtcagcttcagcacgtgcctgaaggcaaacgggggctggcactctttctccttgttggggcatgggtttcgcagcttatcagggtgcgtgttcacgaacggcagcacggtcttgtccacgaaggacccgaagcctgcagggcacatggaggggctgg

SEQ ID NO: 241 - DNA, 400 bp, Homo sapiens
  source 1..400: mol_type = other DNA
tgcgtttagtgtaaaaatatcaggtgtggctgcacggagtgaaaaatcacaggctccacggagccgggaggcctgctgccctgccctcttgctttgatgaggaaatggcgaccgcagaaggaaatgtagcagcaccggcaaccggcatccgtggggccacgccgggctgcttcccagggccctccagccaagcagccacaggaaagagtagatgttgatcccaagctaggactgaggagtccgtccctaagagccgagggagtcaggtgggcgaaactggccgcatgtctgggtacaactgctcagggtttctcatctgctgaatcaccaagctaggttctgaagccaggcgtgagtgagcaggactggagcaggattctgggaacaatcttttccctcc

SEQ ID NO: 242 - DNA, 2000 bp, Homo sapiens
  source 1..2000: mol_type = other DNA
gctggggaactgaaggaagggctgtggagcctgaagcctgggcctggcctgtgctgcggccgcaccgctgggtgatgcaggagccactccacctccctggcaccccagcctcatccggcaacctgggagcgtgggcctcctgcccctccagggaggccctggccgtgtcctcatggggcccctccaggtccttgtggctccaggtcgggacagtggctgtgagatctgaccctcccgttccccctccaccaagtaggagaaaccccggagcatgagccctcgtccttcaccgtcccggggacagggggacccccagatgctgcacggctgacaggccaacgtggcagaagctccagcttcacaggaagccagtgaccatgagagtctgtagctgtaacgaagccacagagctgtggctttctttccccttcagctctaggaaaggttatctgccctgcacagatctccggaggcctggctgggctctgagagcatcagactgattatcgtaagaaaataatctctgcagacacattccttgctagaagcaggggacaaagcccagcttcaaagacaattccacacacgccctccctgccctgcacagctgcctgccgggtgggagcagagcccttgcagccgggctcaggggcctgggcagggacagcgtgtggcaggggcacagctgagacaggagcctcaaagcgacaccaacccgacgtgaagctacagttgaggagacacagctgcccccattcccgggcctcatctccacagtgagacgctggactctctccctgacccaccgtctcttagaacctcccctccatccggagcagttcggcagccccagggcagccaggggaaccctgccgagtgcctctgggccgccacagaccgcagagcccgcgggagccttgctcacacagcctcaggtccactgtggtcttgggggaaagccctgtcctgggacaggggagccgggggtcctggccctggaccaccatctggggaccacgttgtcacgcctgcaaagctccctgccccacccccatgtgccggctggtgttgacacctttgtagagtgggaacctgcctccgaccccagcctgcagccacagggcaggttatagaccaggtgagagggcgccgcgcccagaaccaaggagcacaagtccgcagtgcccatgagatcctcatgctggccggcgcaggagccatcctcggcctctgcaggtcctcgtgggaaaccgcgggggcacgtggggcggctgcagggtccgcaaagccggctgtttgcgaagggcgcagctccacctggaacagccgaggccgcccacgcgcttcccgcgggatcagagcagcctccacggctgttgtctcaggcaccacgggatgcctttcttcgtttcaatagctgtgggaaagcctcaatcggtcctgaaagaacccagatgtgcagcaatgacaaggccttctctgagactctagaaccttctgccatctcagacaggagggagccgtgaggcaggcgggagatttgcagtcagcaaaggacgggcaggtggggcagctgcacacccagggccctctccacggtcttcccgggcccacccctcccgcggtcctgggtcatccacctgctggcctcactctgcccacgcggccaggtcccaccggcccctgagctcaacagaccaaagctggcccgaccccacccccaagaagaatgaaacaatttttttttacctcttgcagaaaagtaaaagatcatttattcattctgtttctagatagcaaaactaagtgtcaaaagcaccttctgcacacagtctgcacacactggccggtggtcctgttcccgcaaggttgagctgtgttccagagacatgggtcctccgggtgatgaggagccgctggagggccctgagctgcacgtgctaatgattaacgccccgtccgtgctggccggtttctcaaatgcctcctgacgattgcgc

SEQ ID NO: 243 - DNA, 2200 bp, Homo sapiens
  source 1..2200: mol_type = other DNA
ggcctgaggagtcaaacggtgcaaaccctgccccactctgtttgggaagcacctgctgtgtggcaggcgctgcgcttggtgctggggatagaccatggggaagaaacacacagaacctgccctgctctcaaggaacaggccctgggggcggccaggggcagagacccaaggcagacacccacacagtggcgtaatgacagtgcttatggtggggacctggctgcacagcaggtcagcaaggggatgttcaggtgacactgggggcacggagacccaggggagagtggattgacagaggggacgctgggcaaatgtcccgaggctgaggtggagttgcgggaaggaggaggctgccgggcagaggcgcagagagctttgcaggtgttggcagagaccagcaggccctgcgaggcctggggtgtgtcctcagctgggagggccatagaaggatctgggcttgcagatgctggtgcagactggaggcctggggtgtgagagtccaggcggggctcctgccaacacccaggggagtgggcctgggccaggtggaccgggagctggcacggtggtcaggtgcttggaggctgcgtgccacgctggggacctggaggtgtgtgaggaggtgtctgttgctcctggggctgccgcctgcagggctgggtgtgcagcagtgcggggcaatgaagtgggcgggttctgggatggtggacgttccctttgttgggaacgtgttggtgccaagctgccatttgagtttggctctgaggggtctgggcaggggacacacagggaatcacacaggatggagtgagttcccagggacccagggtggcttggcctgagaacagctcccactcccagatgtgtgggaagccctcggcaccaagcctcagcctctccatctgtgaaatggagacaacgtcactggacttgcaggctgtccatgagggtgatgcgatcagaaagggtggagttcctgaacgccccggggtcggggtctcacagcaggagcttagctggtgtcggcatctcctggacccgtcctcagctccgagcgcccagtcctgccacctgtgtccaagtctgcactgtgcccacgaggccctcaaggccgcagacagccccacacttctcggacgccgccccagcacggtccttgtgtgaggtggacactccttctggacgccgccccagcacggtccttgtgtgaggtggacactccttctggacgccgccccagtacggtccttgtgtgaggtggacactccttctagggaaggagtagtaactcttgggtggtcgggtagttgccatggaaaggggcagtaatgcccaggtattgccgtggcaaccgtaaactgacatggcgcactggagggcgtgcctcatggaaagctacctgtgcccctgccctgtgttagctaggcctcaatgtggtccagtatctgagcaccgcctcctgcctcagatgttcccgtctgtcaccccattaccagggcggcacttcgggtcctttccagccatcattgtcctggcattgccacagtggacactgccacacaggcttgtgtgcttgcgcgtacccaggtcctcacctctctgggataaaccaggcacgtggcggccgccccattttccacccgccagcggtggaggagttgcccagccttgcaggaaaacagctctcatgccagcagcggagcatcctattcaagttttctcagggctgccagcacaaatgctgcatgccgggcggcttcctcagcagaccgttgtttctctgcgtcctggaggctggacgtcccaggtccccgtgtggcaggcccggttcctcccgcagcctctccttggcttgtgggcggcgtctcctccctgggtcctcgcagggccacccctccgtgtgtctgtgtcctccctccccttataaggaccccaggcagactggatcagggcctgccctaaggactgaattttaccttaatcacctctttaaaagctgtctccaaatacagtcaccttctggggtcctggctgttagggctttgatgcatggatttgggggacaccgctcagcccctaacagcccccatcctctgcctgcctttaccatggggctgagcccagccctgcaggagtcccctggtttgatgtctgctgtggccacggcgaccctcaggctgctccagccgcacttgtgctt

SEQ ID NO: 244 - DNA, 1600 bp, Homo sapiens
  source 1..1600: mol_type = other DNA
ggggagtctccaggggctggggctggagccgcatcagagaggaaaggggtgtttgaaaaaggggcagggcctgggacccaggaaactgttcttccagagacacccgtgaagctgagctttgcctctcagggaagctgtgaccccacgggtgctgcccagagagatcgggccaggtggagccaagatggactggaattccccgacggggacaaggggccggacgaggctgacttgccctgtctgatgaatggtcaggtttgctttttctcctgaaaacacgaggcagtgatcccggccagctaattccagcagactggagacgggatggtggagaatgaggctgtgggcgggaagagcagatgggactcgccagcatcctcacggcagggccgcgctattgccctccctcccctcctactctctggggtcccaggagccccagatacgcaatgctgccaggcgatttctggcgccccgcagacccctgcccctggagttgggccaggtcccggctggagcaaagggggctccttcaagcccgctcctccctgtcaaacccgaggagcctgacaggcgcagcgtcaccagcgtcaccgggccatagtgagcggccaagccagcgtcaccgggccatagtgagcggccaagccagcgtcaccgggccatagtgagccgccaagccagcgtcaccgggccatagtgagccgccaagccagtgtcaccgggccatagtgagcggccaagccttggtctgccagagccggccgcaccagaaggatttctgggtccccagtcctggaggagcacacggtttacaccaggccttgggaggggaagaggcaaggcgtgggcccagccctcactccccaggagaaaccctgtttgagcggcagaggagactggagagaccccagggcggggatccctgagaggagagaaacccggaattcatccacggaggcgttcacccagaggagacccggagcttctccaggagaggctggattgctccaacaggggccctgaggagctgatggcaagagcggaaggcagctctgactcgtgcgtctgactccaggtgtggccgttggggctacagtgggaccagcctgttgtcactgaacccacaaagtgcctccgagcgcgggtggagagagggggacctcccaccgtctgctggccttgaatcttgaatctaattcccgtctgtgctttgatgggagaggcactgggagcgggcggctttttcagttccttttatcttgaatggcctttgggggattttcacagattctgagttcaaagcccagggaggtgtgggaacgtgacattcctcaccgcattcctcaccgcattcctctgtaaaccaggcggtgttggcacccatgagcctgtgtcttctatgacatcaggagttttatccctcacgtcagaaatcagggttccaggcgccttggtttttcttggcgccagcggcttggctatagaagaaaaactgaaggggccaggtgcggtggctcacacctgtaatcccagcactttggaaggccaaggcgggtggatcacgaggtcaggggttcgagaccagccaacatggcaa

SEQ ID NO: 245 - DNA, 7000 bp, Homo sapiens
  source 1..7000: mol_type = other DNA
gctcctcagggggaggttcggggcctttggtctctggacttgggcagcagaaaggaaacatccctgggggcctgtggtgacccccatcctccccagggtggtctggcaggggacactgttttccaaagcaaagccagagcgccaagggctctcgggattcacgagatccacatttatcccaagttagaacagcacatctgtgcgtgcaaacttcattctgacttcggccggctgtccttcttgcccaaagcaccgtgaggcctcatccctgcatccctgttgcttctttcatgtgggatgagaacccaggaaggggctgagtgtgactcctctggtttttagagagcactgcccccgccccgccccctcctgcttccccaccttttcacagttgcctggctggggcgtaagtgaattgacagcatttagtttgagtgactttcgagttactttttttctttttttgagacagagtctcgctctgtcgcccagggtggactgcagtggtgtaatcttggctcactgcaacctctacctcccgggttcaagcgattctcacatctcagcctctggagtagctggaattacaggcgcccgccaccacacctggctaatttttgtgtttttagtagagatggggtttcaccatgttggccaggctggtctcgaactcctgacctcaggtgatccgcctgccttggcctcccaaagtgctgggattacaggtgtgagccaccgagcctggcctggagttattttgggagagggcagcccctggttcagcgtggcgaggctgcgcttgctctcccgggcgggcgtccacaccctcctcgccgagatggagaagcccaaacccctgcagcgctcccccatcacgtccggccctggaagcccccggaaaccctgccacgccctgagtgggagagcgcaggtccctttccggccctggaagcccccagaaacccttgggtgccaggcctggccgggacagcagcgacactgcatgctcagcccttgcgtgagaccacgggagtgtccgccctctgcacgtgctgctgattgcccacttcgtccagcaggtttgggagcttgtggctgcatcctcctgcagacacttgcccattctggggcctcctctctgtcttttctcctctgttgaggggtctgggagggaggccttggagggtacccatgctgctgggactgatgctccccgcggtggaaggagctgcctcttgaacagcagggggctgagcagaggggaggggatgcgggggtgccgtgcacacaggtgctctcaggacgcaggggcttctcagccctgctgtcccagggctgcactccagcagggcagactcctgaggtgcagacaccccagcttcacgctcacacttctggaaggcgatgtctgtgcgtttgctttctgctgcagtttaaaaagccgggctctctccggagcgtgtgtagggcctggtcactggaatatctggactcagtgttaatggcagccacgctgggggctgggcccagctttctgttctccgtgtgggtgccatatccacctccatcgcagccctttctctctcgaccttttaaatcacagtgtcacctccccctgctgtcctgccagtggcccctggaggcttctccccacccctttcttctggggcaattcttaaggctggcattgaatcaggaggccagatgtggcccctagtaactcaccagcagtccctgaggcttctggctcccctggcccaccagcctcccatgtctgcctcaggcctcttgacccgcctggcactgaccagactgtgtgcccgggtgccgtgcccatgggctccgcctcccccaggcaggccccctcttgctccgcggccacccctgctcttgacctcacacctctgcggtgtgtctggacacaccagcaccacggcgggcggggagcggaattctccaggtggggtgggcaggccggcgggtgttgaggtctctgtgcatgcttgtgcgtaccctggactttgccgtgaggggtggccagtgctctgggtgcctttgccagacaactggtctgccgggccgagcattcatgctggtcgccatcacgtgactcccatgcgccctggccctggggttgggtctgcaggactgagaaccagcggaaggggggcgaggcctcgggaatgcgccggcaactggcgatgagctcaggcctgactaatgagcccaggtgactcatacacccggggcctggatgagtctgactgggtcaggacttccctgcttgttctgtcctgggagatgttgtccctggccctgcagagccgggaggacacgaggcctcctgggtcacagccaacgcagcctactcctgcccactgctcgcgccggccaaggcccgtcggcaccacctcctccatgaagccttcctgactgcccccatccctctgtgggcagctcgagtgtgcatcttgagtgctgtgcaggttggggtccggcgctcctgcaggcaggcggcgtctgggcctgggggctctcagagtttgaggagcgtgtggtgagggtggcctcgggcctcaaagacgcagcgctgtgggaaccgggagactggctgagcccgctctgaggaaggtggggccaggggcaccctcagctgacccggcgtgcaggggtgaccagccaggcgtggccaaggatggggtctctgggatcaggagacttcagtagcagccaggaccgaggccaccagtttccaccctggcattttccatcttttgaaggactggaaacgattggattctttaacttttttaagttgaggtgaaattcacaacgcataaaattaaccatcttaaagcgaacaattcggtgacatttagtacagccagaaggctgtgcagccatcaccactgcccaactctagaacattcacacgccggagagagggagccctgggccatcacgcagccaccgcccggccccaagaacctgcgagtccactttccacctctggatcggcggttctggacgttcatgcaggtggttcccgcagtgcgaggccttttgtttcgggctcctctcacaagcctcacgtttccaggtacgtcgtggtgttgtgcagacccacaattcatcccttttcatgggtgtgtaatagtccaccatagattctctacgttttaaagcatgttttatgtgcctgaaatgtctctgcactcgagactatagcttgctttctttcttttctttttttttttttaatttgagacggagtcttgctctgttttcaggctggagtgcagtggtgcgatctcggctcactataacctctgcctcccaggttcaactgattcttttgcctcagcctcccgagtagctgggactataggcgcgccaccccacccggccaatttttttgtatttttagtagagatggggtttcatcatgttggccaggatggtctcgatcttccgaccttgtgatctgcccgcctcggcctcccaaattgttgggattacaggcgtgagccaccgcgcccagccgagactacagctttctttaactgcatccctggagggatctgagagtctctttccctgtctcctttcctttggaaaacatttcagccagggctccccaagatgaaaggccagagtcccaggcatgggcgttgcaggtgcacagttgccacggggagctgtgggtgatggtcgctgtcagcgatggctgctgcaggtccctgtgaggaaggggcagtgccacagcaggaggagagggagtcagcggacgttgattggcagtgcccgcccattccatcattcagtcacccactgtgcacccagcacccaggctcggctgcatagaacatggcccaggaaggctccacttcctgtctcctcttctcccctctccagtctcatgatggggctggaggcatcttctagttttgagttctgagctaatgaacatgctcatgagcaggcggcaggatcccaggacggtggagctgggagcctgactgcgggtgacggacaggctctggcagcccctgtcagcatcctctccagggcatgtgaaagccagtgtgtcctcagctgccagtgccccctccccacctcctctgggcccatgtgcacgggacctgggctcccccaaccaagcctgcccgccttggttcagcagaacggctcctgtctctacagcggtgccaggccaggagtgctgtgtctgtgaagcggggtcatggttttggggccctcatctccctcgcgccctctcattggggaccccccgtctccctagcgccctctcgtcctctcctgcatgtgctgtgtctgtgaagcggggtcatggttttggggccccccgtctccctagcgttctctcgccctctccagcatgtgaagtggggtcatggtttgggggcccccatctccctagcgccctctcgttggggaccccccgtctccctagcgccctctcgccctcgcctgcatgtgctgtgtccatgaagtggggtcatggtttgggggccccctatctttctagcaccctctcgccctctcctgtatgtgaagtggggtcatggtttgggggccgccatctttctagcgccctctcgccttctcctgagcgtgtggaactctgtggtggtcagagctaaggttctgaataggtcgaagcacctccccggtgcctctcaccctgaatgctctgggaggacacagccttttcataggctacgactgacatggcaggaggggcctgcctgccacccgggtcctctgctgcctgctgcttgctggggagggggctcgagactgggatcctgggcttctgctccagctgtgcccaagggagctgctgaggagggaccgggtggggcatccactctgggcaggttcagggtcattcttggtgaccccgggtccggttacaaaggctgatggagcgcgtgggtggctgcctaagtctctggaagcccaagaatgtggagatggcgcgtctcggcccggggtctcgtggctggtctgggagaacttgcctttatttctaggcaggaggctgcactgcaagggagcgtcagtggcccggctggctttccccggccctcagcccgcactcgtccaccaaagcaagctcctttgtggggctgccctgggaagccgggatcacgaggctctgccggccgtggtcaccccatgaggcagggtcagctcgggagcaaggcggatcagatggaacagaacacgtagaccacctcgcccgcccttagtcagctgggccattgaaaatcaagtccgtagaaagacctagaaataagtcccggggtgcccttgcctgttgacgggcgggccgagcaggactgttctcaggcaggcactggtctcttggcttccaggtggtttgtttgctggtttgaggctgggggtgacgctcctgtgcgggaggaggtcgcattccattcatagcggcttatctgggctgtcaggcaggcctgggagggagcctgcctctgtgctctccaagggtgggcgacggacagacagggtgtcccaccccttctgggccaaggacagagggtcagtgtttgcagagacctggggaggcccaggtgacctccaccgagcacctgctgtgtgcagggccagtgctggctgcagagacagcggagcgtgtgtggacccggcggcccaggggaggggggcaggcaggacccggcggcccaggggaggggggcaggcaggacccggcggcccaggggaggtgggcaggcaggacccggcggcccaggggaggggggcaggcaggacccggcggcccaggggagggggcaggcaggacccggcggcccaggggaggggggcaggcaggactcggcggcccaggggaggggggcaggcaggaccaggcggccctgggggtcaggggtggaggccaggcctagacggcccacaggagggtggactcattctgaccgattcctggaagcccccggaaagtggtgatgttctggagggcccagcagaccccaaggcccccaagacaatcccagctggctctctgcggctctcggtgtctgccatttgagacaatttgggcacaggcagggcaggccgtcgcggacggtctaagccgcgcgcattggtgggggcagcagagcccctgctctcagctcctcggggtacagcgggggtaccaggcgggtgagtgggtgggtggtcactgctcctgccaagggcagccctggtttggtttgcacttgctgccctggtgacggctgctctcattcctgccccattgctaacaagggtgtcataagctactttcccggcccacatcctattaagcccatggagaccctcccacagctgagcctgctgtgggctgcaggccctgggcggtgcccacctcggtccccactggcctccttccagcactttagagcagacacaggttggagataaggaaagttccagagcacagactggaacaagccccaggcctctccctgccccagcagggcctccctggatttgggggacaggtgccctcatggggggtcctgaaggtcagagctggggctggggctgggctggcggaggtggccttggcggaggccacattccagggtctcagtgagagtctgtggcaggcagccttgcagatgccgctgagggaccccccacttcatgttgtgggtgatgtggtccattgattgcctccaggtttaaatcaggtggatatttacctagcggcctcctctccctctgcacagggcctggagtgggatggactggggtgctcagctggaggctctgcagacacagccccctgggctatgcaggccctgctgggagccacattgccatttttcatcacccactttttgggtgagaaccccctcgagtcctaacatctgccgcatctcagagcctgtggctccagtcagagcatctggaccatactgctggggtcagagcgcggcaggacaatggc

SEQ ID NO: 246 - DNA, 1500 bp, Homo sapiens
  source 1..1500: mol_type = other DNA
tgccaccaccatcttcaggtagagcttctctctcctccttgctgggcggggcccctccctggggaagcctgcaggacccagacagccaaggactctcgcccgccgcagccgctcccagccagcagctccaacgccctgacgtccgcctgcgcacgccacttctgcaccccctggtgatgggctccctgggcaagcacgcggccccctccgccttctcctctgggctcccgggcgcactgtctcaggtcgcagtcaccactttaaccagggacagcggtgcttgggtctcccacgtggctaactctgtggggccgggtcttgctaataactctgccctgctcggggctgaccccgaggcccccgccggtcgctgcctgcccctgccaccctccctgccagtctgcggccacctgggcatctcacgcttctggctgcccaaccacctccaccacgagagcggcgagcaggtgcgggccggggcacgggcgtgggggggcctgctgcagacgcactgccaccccttcctcgcctggttcttctgcctgctgctggtccccccatgcggcagcgtcccgccgcccgccccgccaccctgctgccagttctgcgaggccctgcaggatgcgtgttggagccgcctgggcgggggccggctgcccgtcgcctgtgcctcgctcccgacccaggaggatgggtactgtgtgctcattgggccggctgcaggtaactggccggccccgatctccccaccctttcctttttgccttgccaggtaagtgtgggcggggctgacgtgagcctggtacaggttccccccacatcgaatctctacgttcaggggcccgtggccctcgggaggtgggagagctgggagtgaggcctcctgtgtggggaggaggccggcgtctggacaggaagagggctggatgaaccgcagccgatgtgtccaggtgccacctgggcctggagctccctgagcattttagcgcatttagtcctcagcacggtcccgagataccctgccatgccccgagtcacagaggggaaactgaggcgtggggcagtggcgtgactcaccccagggagccgagattcccgctcaggtgtggctgcatcgaccttgctccggtcactaagctgcacggttcgatgcgcttcctgggagccccagcgtgctcgggccaagggtgctgccgcgtgggcagtgcagagaccctaccagcgtggggaccagggaggtctgcagggcccgtcctgagagggagcctttcatgtccccctccccatcctgaagcacacagcctccctgccacagtgggggccgcttctgggcccaggggacgttgccccatcaccgtgtggcctggccttgttgctggctggacagttgggggcaggaagaggagggaaagggggactctttaacctcctgggggcaggggcagcccagaaaggaccccagcagatccctcctctgtgtccgggagtagacggggcccc

SEQ ID NO: 247 - DNA, 2000 bp, Homo sapiens
  source 1..2000: mol_type = other DNA
gggctccacagcggcctgtctcctcacagggttcagcccagtctgctctcactcatttgctgattcattctttcattcagccagtcaatagtcatggcccctcctgtgtgccgggtggccatggatattgccctgggtaacacacagcctggccctgtggagcagacagtggggacagccatgtggacagggtgcaggtggatggcaatggcagctgggtcaggaggggctgagggccgtggggaaaggtgcagaatcaataggggcatccggactggggtgcaggcctgggggctgggatttctagggtggaggtcacctctgagggagacagagcaaggccctgggagattagaaggtcgaaggtcgccgtgttgaggtcaggggccctgaattggagccgcggcaaaggagagggcaggtcagggcacgtggtgagtgattgctgcggcttctgagcacggctgggtctgtggggcctgagcagaggtgacccgcgatccggcgccacggcaggcaggactccccacccttgctgctgcctacacccccagggcagccccagagtcgggggcgcagctccctgcttgccagttcagagcccagcccctctcacccagcccagaggaggacacagatggaggaggggcacccggagggtccccccgccgacaggccccacgtctcccacctgcaggacaatgaagtggccgccttgcagccccccgtggtgcagctgcacgacagcaacccctacccgcggcgggagcacccccaccccaccgcgcggccctggcgggcagatgacatcctggccagcccccctcgcctgcccgagccccagccctaccccggagccccgcaccacagctcctacgtgcacctgcggccggcgcgacccacaagcccacccgcccacagccaccgcgacttccagccggtggtgagtgcccccccaaagtgggcttggctccatctagcccctcggctctcggcagcagaagagggcccagcccctgcagagctgctgggggtcccaggcttcggccatgggtgggggtctggcggctcagggccactcagggcggcttggctggccctgggacttgccctctggtggccaagcagtggtcatgaaagtccagccgctgtcacatccttgaggaaccggcgtacctccgcctacagcggcagctgggggcacccacgtggcccggggctgctctgacctggcagcgtatgggggctgctgcctgggcccctcagtgtgtcacttgcgcgcctcccgctcagcgcccctcggccgtgcctgtccacacaggtgcggggccggggtggtgcgcccggggcctgggtgcagggggcagcgtgggacacagcccgtgacgcgcccctctccccgcagctccacctggttgcgctcaacagccccctgtcaggcggcatgcggggcatccgcggggccgacttccagtgcttccagcaggcgcgggccgtggggctggcgggcaccttccgcgccttcctgtcctcgcgcctgcaggacctgtacagcatcgtgcgccgtgccgaccgcgcagccgtgcccatcgtcaacctcaaggtgggtcagtccagtcctgagggcgcgggctcctcggcccccacttgacctctggggtgaactcccagcggggagctcccctctagggcctctggaggccaccatgttacagacactggcgcctaggctggcgacttcagggcaggctccgggtgggtcacacccctccaggctcaggccaggcctctgcatccctgggcactgccacgtcccccagggcatcccatgaggcccccccgtggccccctgaccccccgctcccccggcagtgcccctcagagggtcccatgctgctggaccaagtgtccacacaggtgatagggctcacatacaagcctggaatcaggaaccgtcctttgggcctctagtgccatgcgggctggtggcccctctgcca

SEQ ID NO: 248 - DNA, 2000 bp, Homo sapiens
  source 1..2000: mol_type = other DNA
gcctggagtgtagtcctgctgaaggccagagaccacacactccacccagactccggatctccctccccagcagggggatggaggccctgccgctgggagtgctggtgttatgtggaagggctgggcttctccagggctcctgggaggcctaaacatcttgcaaggttttgacgttaattactattatgattgctttctgtgtgttactgttttccccacactttagccagctaatgtggagctacagaaggccctcgcccctacccctccagatgtcccagcccatgacaagcaggaaggccgggtgctgggagacttcctggggctggatctgacatcattccaagcagatgataacctgccttcccgatttccaaacccacagcaagacaccctggagttatttataaatgcgagcccctgggtgcacttctgacgggaccagcaccctgacggccatgagagggtggagacagcgcaccccgagctcagggaggcaggaaactctggacctggaggccgggcaccatgagggacacgctgcaggcccagctgctgccgcctggggcggggctgccctgcaggctccgggaaaacccagaaccaggccggatcagcgtgtgtcaagaggcggggcgtgagagatgagctgctttttttcttcacagggttggcaggaactgcaaataatagaaagtctttagggtctaacacgctgccctgaaaacactatcattactttcctaatgactaactgtgtctttcagccggcggggcaggcagctgaggccgcaggctcccgcagaggaccgggggaggctggcagcctgtaatctgggggcgctgacagtgctctgcccagaccctcgcgccagctccagctccagcacagcagccctgggtccctctggccccctgcccgcagagtccaggtgtggcagaggccgcccagtatcccttctcctcctccttttctaaaaacagagtctcacgatgtttcccatgcgggtctccaacgcctgggctcaagcgatccttctgcctcggcctcccaaagcgttgggattaaggggcgagccaccgcgcccggcccaccttcccttctggttcatttccagtaaggtcctgtccacagcgtccttcccagcattcccaccaggctgcaggccttggcctccctcccctccattctcattctccccgaaaccgccaagcgcgtccaaagcacgggttcgccaagcgccccccccgccccactccacattcccttccccgccgactcagcctccgtagctcgcggacggcccctcctcacgccagcccaggctttttttttttttttttcttctattttaaggttgtcttttaatgacacaagcgacatttggagacaaaaggacacatctcttcctgacccacctccaaccccagctgacggccgccctgagcctggcgtagacggcccggaacgttccctgcgtgggttccgtccatcccgaacccctgtccccgcgccggctccgggggtgctcggggggccgcgtggggtctgtgacgtcgcctcgaggctgcatcccggtgacccggcagcccctggcgctcgcgggaggcgggcgggcgcggaccccaggctttagggcgcgattcctgcagctggctgccggcccgaggttctggggtgtctgaggtctcgggcggggcgaggacgtttctccggctcagcccccccacctcctgccctgccgccccccacacccagctccccacggacgccaagaggcgcctcccaccccggcgaggacccgcggggaaacggggcccaggcgcggcgactgcggaggacgcgcctcggccccagcgccctggtcctcggggcgtccggctgcccttgcccgaggccggggcgggcgctcagcgccgcggaagaaacgcccgggcggggacgcacagcgaggcgggctccgcgggaagtaccgggaaaacggcgcggagcggaacag

SEQ ID NO: 249 - DNA, 3000 bp, Homo sapiens
  source 1..3000: mol_type = other DNA
tggagcaatcccagagaggctgaggtgttcaggctggccccagatgcacacgagcgtgaagcctgttcagaagccagctcctcacaccctctcccctgccagaggctccagcaccccctcccctctcctctcccctcccttccctgtggtcctcctgcccaccccacccccgtctgcatgtgcaccgtcacggagatgcgtgtactagggcggaggtcggggacagtcgtcagaaggacacaggaaagaagggaacaggaatcccataacagaacattatccggcaggagtaattaacacaggcaggactggaggctttgttttgttttgcttaaaaaacagtggtatttaaattaatgggcatgggaagactattcagtgaaagacatcggtcattgaggtatctattcaaaaacacggtttagtactctgccacacaccgaacgcaacgccacagcagccatagaagcgtgtgtggctgtttaacgtggtctttttggggagggcatcctaggcagagcaggcgtggaagggaaggcggcggacggaacaaaacgcgggcacgcaacggctgctgcgccggatctgaggcagggccagcctgtgggagcagcaacatcgctcgcaggacagcgatggagcccccacgaatccgcgtgaaagcagcaaccacctagaaatgaacgtacagctgcttagaaacagaatacggatgacccgaaagacttcccgatggtagtcaccagcatacaggacctgacacgggcgtgcgggcagggtgtgccgctacggggtccctggcgcacctgctacccctgctacccgcattcaccgcacgcggagggtgcgggccgtgaaggttatacatgcaaatatccttccaccagccagttctccttccaggaatctgccacccgacccttgtgttgtgcacagacatggtccaggtgtttgcgacgtgattgtttatcagagagagagaagggaaatctccaggctcgctgtagctgcaggagctctgggggctgcgcccatcgtggagacggatagctgtctctcatgaacacaggacagcaagtccggctgcggccacagaagactcgccctcctggacgcagcgtcttccttcctcagccccacactggaggtggccagtgccatccacagcagaaggggccagccgggaccaggctcacgccgtggaattctgctctgtggtaagaggaagagcgatagctggaacccagcgccgtcgcacacacagcggggaagagtctcagaaatgttactttgagtcaaaaagctggacaaaaaaaggcgcaagccagatggtgctgaagaggccacaggaggctggcagccagggggtctggcacctcactcggaggcgcagtgggcccgtccggaattagtggccatacggcaagtgccgagtggacatcaaaccgtcacttcagactcctgcgcttcactgcctgtcggttatgcctgggttttgaaatcaagtcacagaacacctggaatgtggtgtttacgcagaacaaagcgggtgcctcggaggagagagcctagggacaggggcacctcccggtgtgggtgcccagggttgcagggtggcttcctctgtctgcgcggttttcagagccccagggtcctgcctgcccggctgcctggaggcggcccacatcctgctctgcgccgccgaatctcagcctgaacagcttcgctggtgtttgtgttgacttatttgttctttttttttttttttttttttaaataaaggattccgatgctgttacagtcaataaaagccacaggtctgggtgacctacaaatgtgtgtgtctgactttctgcagtttaaatcgccactgagccttaaggcgtctggcccgcgcattgaggaatccacgtgggtctcggggtccccatgcctgcccagctccctgcttcagcctgggcgggtctggcgggcatttctgcgagcctgtccctgggcccgcctcctggccagacttccagaaacattgtccacatccccgttgcacgtccccccgtcaccggaaactgcagcccacagcactgggaagaacccgggaggcaggcgttaggacggggtggccgagacagggaagggagccatggcggacgtcctcacccaagccagggcttcctgcccctgtggtactgacaggagccccgcaggacgtggggttggctttgggcagctcggtggacacttctctttcagatcctgccacagcaaagctcacgagactcacttcttcccattggaattcactaagaacaaattcaacaattcagacgccccagctggaggtttattttatggattttacctgtgcggtatttagggttgtgtttatgaataaaggtgtgcgttctggcaagtagaaatacagagcttgtctttcacccaagtatctgtaactttctccaatgcagacactaaaatgcaataaaaacaaaccaaacccattaaacatgaattagatgaggcaggctgatgggaggttgtgggattaacaggccgtcagcggattgaagctgcgcacatcgctgggatgctgctgcgggaggattcggtctaatccgggagcatctggctgggcagtgggcagcgtctgcagtcgtggctgcttgaaggtatgaaggttgtggcctttgcttccccccatcaggctgccccaccctggaccccacccagacccctcgggcaccctggggtcatcttcagctcccccttctcttccttccttctcttccgcctgggcccctactgtgacccgaggtcagcagaggaccctggcaggtggctgctccctgggactcgactgtgcaggtgaggcttggggtgaccgctgctcctgctcctgctcctctcgccgtccccaccctcctccatcatgctgtcaacatgcatgtgggctgcagccctcagcctgcaggacgctgtcagtgcagctcctcagtggccagg

SEQ ID NO: 250 - DNA, 2500 bp, Homo sapiens
  source 1..2500: mol_type = other DNA
atcttgtcttccttgtcccagtcctggaaccagccactgccccagcagctcctgtgtgtggtggcatgttctggaagccaggatgcatggtgctcctgggctgctgtgggtcctgggctgctgtgggtcccgagctgctgtgggtcctgggctgcacccctgcagaacacttccttccatgttcagctccctatatggaaccccagttccagccccacagcacagggtcccccagttcttcctgcctcaggtgtgcaccacgaggaatccaactgccagtatctgtgcgtggcctcccgccgggaggaggctgccggaggctctgagctctagccccacagcactggcacatcctagatttccgggaagacacggcctcctccccaggggaaggtggtggtgcccacacccagagcattcattcctgcagtggagacagagggacctgcctctccaactgtgggtgtcaggagccaaggcgcatggtaaatggggctctctgtgaggccaggtgcacggccccatctccagcagcagcggccatgccacccagctgcactctgtgggggaggtgccatgattgacgggggcccctccctgtgtccagtgtcctcctccctccacgggcccctctgcacaccgtcctcacagtctccctctgcacaccgtcctcacagcctccctctgcacaccatcctcatggtctccctctgcacaccgtcctcacagcctccctctgcacaccgtcctcacagcctccctctgcacaccgtcctcacagcctccctctgcacaccatcctcatggtctccctctccttccacagacccctctgctcgccatcctgacggcctccctctccctccacggacccctctacacactgtcctcccagcctccctctacacgccatcctcacagcctccctctccctccacgggcccctctacacaccgtcctcacggcctccctctccctccacgggcccctctgcacaccgtcctcacagcctccctctccctccacgggcccctctgcacgccgtcctcacggcctccctctgcctccacgggcccctctgcacgccgtcctcacggcctccctctgcctccacgggcccctctgcatgccgtcctcacggcctccctctctctccacgggcccctctgcacgccgtcctcacggcctccctctctctccacgggcccctctgcacgccgtcctcacagccttcctctttttccacagacccctctgcacgccgtcctcacggcctccctctccctccacgggcccctctgcatgccgtcctcacagcctcaccgacgtcaccattgctggccccgcttcaggtgacaggccacagtagcacctgtcagctctgtcccgctgctggacagggagatactgggccactcagcccagcggggaacgtgtgtcccgaaactgccttgggctcgccatcagaactgtggcagcatcttccagcgttccttttaacaggctgccgttggaataggagtcacggagcaattgcagtgctaagttttctttaagtcacacaattgaaggaggctttatttttcacacatttcttccagagtttcctggtagcctgagtgcatgggtgatgccccctgagttatttatcaggggcagccagctgccctcccccggggcacttacagtcagcccatctctgtcctggtcaggtgggcgccaaggaagacccggctcagggcctctgtatgggcagcctggcttgtacacacacccctccccaccagcagattctgaattctcccttcttcatgcacaccgggaaggtcccttctgcactcataccgggaaggtaggcaggtttcggtagtgtctgcctccagtgttttcctcctcctgctctatgacatcatctttctgtgattttttttttcttgcaggaagttggaagcatcatcgggaaggtaattattgattgaatctctgcctctcctggggtctctgtaaggggatggtgaggatggcagcctccctgggtactaggtggcacccagtaggtgcgcctttcccagttggtgggtggtctgtgttccatgaagacaggaccccagaggtgtcgcctttatgctgtatgacattgaagctggtccctggctctgcgtggcctgaggggaaggggttcactccagctggtcacctcgctgccccctgcccgtggccttggtggccagtccttctttcccggttgaagaccccacgaagaatgatttctcacgccttcttcagccggctgtgtagtctgggtggtctccaggagtgccagtggaggcagcagcccccagacaattcctttccaaatcagggctggcccgggggaagtaaggcccagtttggaagcctgctgccccgggaggccgagcagtgagggccacctccctgtcttcatcacattttcaccgcttccgggggtccttcccctcagtcccaccatgggggcgcc

SEQ ID NO: 251 - DNA, 6000 bp, Homo sapiens
  source 1..6000: mol_type = other DNA
gctggacacctctgagagcgtggccctgaggctgaagccctacggggccctcgtggacaaagtcaagtccttcaccaagcgcttcatcgacaacctgagggacaggtaggagggacgccccgtgaccttcctcctgtgcttctgggcctcttggagggaggggtgggggcccaggggaacacgggtgcgacggcctcaacctcctaaggttgggcgagcgttgccctgaccggggcccctcccggcgccctccagagtgaggccggggccctttccggcgccctccagagtgagctggtctgagcctctcccagcgccttccagagtgagctggtttgagaccctgctcgcgggggtggcacctgttcagcagggccgaggtgacagtgaggctgagatgtagggaagagaggctcccgcaggctgaccgagagggctcagcgcactggcccagacacgcagtcctgcctggtgcgcgggagcccctcactaaccacctggaccctggtttgttccgtgggcagtgagagcctctacctgggtcctggatcccacgttctgaaggtccccgactcgggagccaggaggggtgtcgctctgcagccccagggcccccaggcttggttctgggcttgggacacggcaccctctgctccacgttcctccatctgtgcgtgtggctgaggacagaccggggggagaggggagtcggtcctgtgggtgcacagggccgctgaggggggggcatgtagaacggggctcccccactgagacgggtcctggcagtggggacacagcttagccggcgtaggaacccccgtcctccttgaccctgctgactggccgctgggccggagcctcccgccaccagaaggggcacagtcagaggctgccggtaacagcagggtggaccttccagcccacaccgtgcccagcaggagccattggtaccaggaaccctgagcttagtggacatggccaggcccgtgcggcagtgtttgggggggggtctggctgtggatggcaccggggaggggcggccgcgtggcccagcgtcccccgagtcgcccttgttgcctttactcagtctccccatgactcagtttcccacctgtgaaatggggcggagtcatccccatgtcgctgccactggattcctgcaggcgccgtggtcactctgctgaatggatgggagggtgggtggggcagaggtgggcccaccccaggctggggcagagcagacccctgagagcctcaggctcaggtgctcagagggcagcgagggggctgctcagatccccggggtgcctccttcccccactgtcatgctgccccactgcaggcccaaggaccccaccccagcagggccacacactcagggctcctggtctgagggcctgagggatcggggcgcaggtcgcttgctggccacacccgcctgcacagccttccaggagggccggcctcagggccacagggcaagtccagctgtgtgtcagccacggccagggtggggcagcctgtccatctgggtgacgtcgcgccctgggacgggtagcgatggcgccaggggccgcccgcctcacgcccgccgtgcctgttcctggcaggtactaccgctgtgaccgaaacctggtgtggaacgcaggcgcgctgcactacagtgacgaggtggagatcatccaaggcctcacgcgcatgcctggcggccgcgacgcactcaaaagcagcgtggacgcggtcaagtactttgggaagggcacctacaccgactgcgctatcaagaaggggctggagcagctcctcgtggggtgagtggcccccagcctcctgcccacgccagttctcacgcgtggtacccagcctgggctggggttggcctggggtccctgtgcggcttcagctgcagcctccctgttctcttggaggctgcacggcctccctgacccactttgtgggcaggaaagagacggagacagacagagacagagagaaacagaaacagggagaaacagacacagagagagacagagacagagagagatagagacagagacagagagagacagagacaaagagtgacagagggaccaagacaggcagacagagacaaacagagacagagacagagacacagagagagacacagagagacagagacgggaacagagacaggcagacagagacagagagagacagagacagaaacagagacagagggacagagacaggcagagagagacagagagacagagacagagacagacaaacagagacagagagacagaaacagggacagagacagaaagagagagagacagagggaaacagagagagacagagacagatagaaaaagacagaggcagagagaagcagagacagagaaacaaagacagtcagagacagacagagacagagacagaaacagagacagagagacagagacagaggggcagagacaggcagacagagagacagagacagagacagcgaaacagagacagaaacatacagagacagagagacagagagaagcagagacagacagaggcagagagacagagagaagcagagacagggacagagacagagacagaaatagagagatagagacagagggacagagacagagagatagagacagagagggagacagagagatagaagcagagagagagagacaaagacagaggcagagagacagagagagaagcacagacagagacagacagagagacagggacagacagagacagagagaccggaaacagaggcagagagactgagagactgagagagacggggtggttttccccacagcatcaacaccaagcagggctaggatcactgaaacagactcatcagacccgaagcatgcgctttctcggggtttttctggactgaggggtttcctctcatcccagtgtccagctgtggggacgcaggggccgcaagccccggagtgtccagaggggaacgtggcctccccacacccagcccttcacgaggcctcaggatcccagtgggggtacccgaggctgccctgtccagccaggcggtgcggggggtttggggagagcctctccccgaggtcggtctcagagggccacatggccggtgtgggccggacattccctttccaatggttgtgcccacttccctccagagttggtgccaagctgggacctgggggacttggagtctcaggaagtcgtccgctgtctgcagggggtgcatgggggatgtggccacacacgtcagagtgcggccccctgtggaagccacagacagacacgactcccctaaatgagctcgcccttctggccgagatgctcagcgtccccagcaggctgcccgactgccctgcgatactgccctccttcctgctgctcccactttccctttcggggggttggatttggggcattcagggatcgccctgttgtttgctcatcacacccatttcctgcaagagccacggtgaccgagcagccttgagttgaggcagcttgtgggtagacgcggcgggcatctcggaggggcacgctccctgccaccctcagcctccactcactggtcaggggctttgcgccccagggcaccccaggaaccgagcctcctttggggtcatgggtgcctctcctgggagggcgtggattttccaaagcagtttagagaaatgagacccacaggcgttatttcccatggtgaggttcttttcagtaacccccaccgtatagccaggatcagcaaagagaggcggctcctcccggtgagacagggaccagcacctcccggacaggcttgggtctccctccagttcccccacctagtctcgaggtctcacgctgccctctcctgtccaggggctcccacctgaaggagaataagtacctgattgtggtgaccgacgggcaccccctggagggctacaaggaaccctgtggggggctggaggatgctgtgaacgaggccaagcacctgggcgtcaaagtcttctcggtggccatcacacccgaccacctggtaggcaccggccccccccggcagatgcccccaaccacagggagtggcggctgcaaggcccccggcagctgggaccgtcttttggtcctcgggagggtgtgggttctccagccggccacccttgcccctgagaggccagcccctcctgctgaggagcctggagcgccccagcccagcctcccctctggccctgtgggaagcggccccggccgtcaggggtcccagccctgctcagcccaccctgaacactgcccccaggagccgcgtctgagcatcatcgccacggaccacacgtaccggcgcaacttcacggcggctgactggggccagagccgcgacgcagaggaggccatcagccagaccatcgacaccatcgtggacatgatcgtgaggcccctgcccaggagacggggaggcccgcggcggccgcaggtggaaagtaattctgcgtttccatttctctttccagaaaaataacgtggagcaagtggtaagagccctccccaccacccccagccgtgagtctgcacacgtccacccacacgtccacctgtgtgttcaggacgcatgtccctatgcatatccgcccatgtgcccgggacacatgtcccctgcgtgtctgcccgtgtgcccgggatgtgtgtccccctgcgtgtccacctgtgtgtctgcccatgtgcctgggacatgtgtccgcctgtgcgtccatccgtgtgtccgtctgcccatgtgcctgggtcgcatgtcaccctgtgtcccagccgtatgtccgtggctttcccactgactcgtctccatgctttccccccacagtgctgctccttcgaatgccaggtgagtgtgccccccgacccctgaccccgcgccctgcaccctgggaacctgagtctggggtcctggctgaccgtcccctctgccttgcagcctgcaagaggacctccggggctccggggcgaccccggctttgaggtgagtggtgactcctgctcctcccatgtgttgtggggcctgggagtgggggtggcaggaccaaagcctcctgggcacccaagtccaccatgaggatccagaggggacggcgggggtccagatggaggggacggcgggggtccagatggaggggacggcgggagtccagatggaggggatggcggggtccagatggaggggacggcggggtccagatggaggggacggcggggtccagatggaggggatggcggggtccagatggaggggacggcggggtccagatggaggggacggcggggtccagatggaggggacgtcggggctccagatggaggggacggcgggagtccagatggaggggacggcggggtccagatggaggggacggcggggtccagatggaggggacggcggggtccagatggaggggacgtcggggctccagatggaggggacggcgggagtccagatggaggggacggcgtggtccagatggaggggacggcggggtccagatggaggggacgtcggggctccagatggaggggacggcgggggtccagatggaggggacggcggggtccagatggaggggacggcggggtccagatggaggggacggcggggtccagatggaggggacggcggggtccagatggaggggacggcggggtccagatggaggggacggcgggagtccagatggaggggacggcgtggtccagatggaggggacggcggggtccagatggaggggacgtcggggctccagatggaggggacggcggggtccagatggaggggatgtcggggtccagatggaagggacggcggggtccagcaggcaggctccggccgtgcagggtgtggactgtcccgggggcgctgggggcttctgagggtgtctctgtccgccctgccctcagccgcactctgttcagaaggacctttctggaggtaggagggtgagaatgtgggtcccctgcttctgtgtggctcac

SEQ ID NO: 252 - DNA, 7000 bp, Homo sapiens
  source 1..7000: mol_type = other DNA
ggccggggaggcggggaggctgccccaagagtaaaagcctttctgacgtgcgcaggacgcggccctgactggtctaactgactctttctcttctcctcagcttgctgtggtgagacccaggctctagctcctgagagaatggatcccgggggtcggggagcgaggcctgggtcccacacatgtcacaggacagcacatggcactctggtccccgcccgcagctccctgcacctgcccgccccctctggggcctgctccaagccagcagggttcccgggtgttgggctgggccccgccctctttcacccataactgaaataaccaggagcaggcttgggggggtccctgctccatcattctggcccacaggccccaccctagcctggctgagcaacgccagccctgaccagccgccggacagagcagcctttacggggccatgggagggggtgggcttttctggggctgagacggggggaccccaacgtgtcaggtgaggatgtggcagccaaggaggggccagggcggtggaggggaggggccagggcactggaggggaggggcgtgctctgctgacaccgcccccgcctgcagaatgcaagtgcggccccatcgacctcctgttcgtgctggacagctcagagagcattggcctgcagaacttcgagattgccaaggacttcgtcgtcaaggtcatcgaccggctgagccgggacgagctggtcaaggtgaggcctcgccccgcccggctttctcaagcccaggtgcaccccgaccctgccggccgcccctgcccgcgccagacctcagcctcccgaggccaccgctgcatccctgtgacttccctactcatgacaaggatgccaggcacgcgccagcccgtccaggcctccagctccacctggcgaggctggcccattgtacacaggcgccccagatgagggagggtctccccctctccttgaagggcggtagtctggggtcctgagtgctgggtgtgggcttgtccctcgtggacagaacccaggagggcttcatccaccaaggaagattgctttgcagggtacccaggtcccgggggctgtgccaccctctgggcacccggagccaatcgcagggtacccaggtcccgggggctgtgccaccctctgtgcacccagagccaatcgcaggggacccaggtcctgaggtcctgggggccatgccaccctctgggcacccgcagccaatagagtcacccttgggaagcttatgcggacctggggcagcactcgcgtcctgaccccggtgccggtcccacagttcgagccagggcagtcgtacgcgggtgtggtgcagtacagccacagccagatgcaggagcacgtgagcctgcgcagccccagcatccggaacgtgcaggagctcaaggagtgagtgccccacgcggccaggaccctcccacccctcgccccgaccgctgttcccacggcaggtcggccctgacccctgatcccaggtgggctcggccccgcggcaggcctggccccaaccggcccttcctgccctttgctatgcagagccatcaagagcctgcagtggatggcgggcggcaccttcacgggggaggccctgcagtacacgcgggaccagctgctgccgcccagcccgaacaaccgcatcgccctggtcatcactgacgggcgctcagacactcagagggacaccacaccgctcaacgtgctctgcagccccggcatccaggtggggtggccacccccaggctgcacctgccccgcctagggcgccccgccagccagggtggccttgtccccagaaagacgagggcagagcaggctgcgccacaccgatactgtctgtccccacaggtggtctccgtgggcatcaaagacgtgtttgacttcatcccaggctcagaccagctcaatgtcatttcttgccaaggcctggcaccatcccagggccggcccggcctctcgctggtcaaggagaactatgcagagctgctggaggatgccttcctgaagaatgtcaccgcccagatctgcataggtgcgcatggggccacccgggcagtcccagatctgcgtaggtgcgcgcggggccgcccgggcagtcccagatctgcgtaggtgcacgcggggccgcccgggcagtcccagatctgcgtaggtgcacgcggggccgcccagggccgtcccagatctgtgtaggtgcgcgcaggcgcccagggctgtcccagaggcctcctcccagctcactgttacctccaggggcacggccaccctgtaggtgcgcacggggccgcctggggctgtcccacaggcatcctcctcccggctcgctgtgacttccgggggcacggccacccctgtgctcggccgggaggtcctgtgacatctccttgcggggttataggtggagcagtgggctcacactgcacggcttttctcttttacagacaagaagtgtccagattacacctgccccagtgagtacctcggcggccgggacacgtggggaggagggcaccgtggttggggcgagggctctgagaggacggggctctgggaggagggcctggcggtcacgagagtaggtgcatggctcactccggtggctgagcaccaccgtgccgtgccctctctggggagcttagacgctctctggccggcccactgcggctgcatcaccagggcctcatgctaacggctgcccaccccgccccgcagtcacgttctcctccccggctgacatcaccatcctgctggacggctccgccagcgtgggcagccacaactttgacaccaccaagcgcttcgccaagcgcctggccgagcgcttcctcacagcgggcaggacggaccccgcccacgacgtgcgggtggcggtggtgcagtacagcggcacgggccagcagcgcccagagcgggcgtcgctgcagttcctgcagaactacacggccctggccagtgccgtcgatgccatggactttatcaacgacgccaccgacgtcaacgatgccctgggctatgtgacccgcttctaccgcgaggcctcgtccggcgctgccaagaagaggctgctgctcttctcagatggcaactcgcagggcgccacgcccgctgccatcgagaaggccgtgcaggaagcccagcgggcaggcatcgagatcttcgtggtggtcgtgggccgccaggtgaatgagccccacatccgcgtcctggtcaccggcaagacggccgagtacgacgtggcctacggcgagagccacctgttccgtgtccccagctaccaggccctgctccgcggtgtcttccaccagacagtctccaggaaggtggcgctgggctagcccaccctgcacgccggcaccaaaccctgtcctcccacccctccccactcatcactaaacagagtaaaatgtgatgcgaattttcccgaccaacctgattcgctagattttttttaaggaaaagcttggaaagccaggacacaacgctgctgcctgctttgtgcagggtcctccggggctcagccctgagttggcatcacctgcgcagggccctctggggctcagccctgagctagtgtcacctgcacagggccctctgaggctcagccctgagctggcgtcacctgtgcagggccctctggggctcagccctgagctggcctcacctgggttccccaccccgggctctcctgccctgccctcctgcccgccctccctcctgcctgcgcagctccttccctaggcacctctgtgctgcatcccaccagcctgagcaagacgccctctcggggcctgtgccgcactagcctccctctcctctgtccccatagctggtttttcccaccaatcctcacctaacagttactttacaattaaactcaaagcaagctcttctcctcagcttggggcagccattggcctctgtctcgttttgggaaaccaaggtcaggaggccgttgcagacataaatctcggcgactcggccccgtctcctgagggtcctgctggtgaccggcctggaccttggccctacagccctggaggccgctgctgaccagcactgaccccgacctcagagagtactcgcaggggcgctggctgcactcaagaccctcgagattaacggtgctaaccccgtctgctcctccctcccgcagagactggggcctggactggacatgagagccccttggtgccacagagggctgtgtcttactagaaacaacgcaaacctctccttcctcagaatagtgatgtgttcgacgttttatcaaaggccccctttctatgttcatgttagttttgctccttctgtgtttttttctgaaccatatccatgttgctgacttttccaaataaaggttttcactcctctccctgtggttatcttccccacaaagtaaaatcctgccgtgtgccccaaaggagcagtcacaggaggttggggggcgtgtgcgtgcgtgctcactcccaacccccatcaccaccagtcccaggccagaaccagggctgcccttggctacagctgtccatccatgccccttatctgcgtctgcgtcggtgacatggagaccatgctgcacctgtggacagagaggagctgagaaggcaacaccctgggctttggggtcgggagcagatcaggcctcagtgggctggggccggccacatccaccgaggtcaaccacagaggccggccacaggttctaggcttggtactgaaatacccctgggagctcggaaggggagttgagatactgcagggcccataggaagaagtcttgggaggctccacctttggggcagaggaagaagtcttgggaggctccacctttggggcagagcaagaagagggcggagggcagaggcagcgagggctcatcctcaaaagaaagaagttagtggcccctgaatcccagaatccggggtgcacggctgttctgggggccgctaggggactaagaggatcggccgagggctgggctggaggagggcagcagggatgggcggcgagggtgagggtggggcttcctgaaggccttcacctgcggggaccccggcgagcccctcaggtgccacaggcagggacacgcctcgctcgatgcgtcacaccatgtggccaccagagctgcgggaaaatgctggggaccctgcatttccgtttcaggtggcgaacaagcgcccctcacagaactgcaggtagagacgggcccggggcagacgcagtgaggcggtgggcggggcccggggcagatgcagtgaggcggtgggcggggcccggggcagaggcagcgagcggtgggcggggcccggggcagacgcagtgaggcggtgggcggggcccggggcagaggcagcgggtggtggccggggcccggggcagacgcagtgaggcggtgggcggggcccggggtagtcgcagtaggtggtgggcggggcccggggcagacgcagtgaggtggtgggcggggcccggggcagacgcagtgaggcggtgggaggggcccggggcagacgcagtgaggcggtgggcggggcccgggtcagaggcaacgggtggtgggcggggcccggggcagacgcagtgaggcggtgggcggggcccggggcagatgcagtgaggcggtgggcggggcccggggcagatgcagtgaggcggtgggaggggcccggggcagacgcagtgaggcggtgggcggggcccggggcagacgcagtgaggcggtgggcggggcccggggcagacgcagtgaggcagttgccagcctctctcagctgcctcatgggattcgcactgcagctgcggccctggcgcgacaagggctggacttggccagcgggacggtccctcacggcgctgaggcccacactctgcgtggagcctccccgtgcccaggctaccctgcaaggtcctcggagaggcttcctccagccccagcccccacacagctccggcccaggcccgctcttccccatcccagttgctttgcgctgtatacggccaggtgaccccgagccggccctgagccctcgtcccggcttcctcccctgtaagctgggtgaaggactccatggcacccacctgagagggttgtggcgaggcccaggcccctcgtgcccacacggccggcggcccatgcctggcaggggctgggaggaggctggggcgaccagaggggagcggcctgtcctggaggaggcccagggaccctggtgagagggtctctcccaagtgctctctatgggacccccttcctctgcgcccgtccttcacggacctctccgggtcacccctgggctgcacactgggttcaggggggccttgaggtggggcccctgttcccaagtcccggcggggtttctcctgaacctcaacccatcctcacctgcgggcattcccatcccccaacgcctgggtcaccaggattccaggcaggaggggcggtgggggttaccaaggcccgggttgccatgcagaacccccagccaccacgcagacccccacggggcccagggaagctcctggtctcacactgcacctcacacttcctgtgggggcagactccaaggtcccggcctctcatcttgtagaaactgaggcacaggagggacacacactcccacggccggtcaccgtggcccccacacctcccactggactgacacctggccaggctccggacacccgtggcacagcctcagcccctgcggcccctgctccgtggcccccaggccccagctcccatgtgcacgtcctgcctcaggcctggaggcccctcggccccaaataatcagacaattcaacagcaaaactacttttttcaggctggcaggactctgggcaaccccctgcaacagccccctgccctatcacagccacccttgcctcccaggcacggagaccccaccatcaggtcccagccttggttcatccccaagcaccctgtgtgttgggatggcgatgctggctgagcccctgcatcc

SEQ ID NO: 253 - DNA, 2500 bp, Homo sapiens
  source 1..2500: mol_type = other DNA
agggcgtttgggaacacccctcccggaggggtgaggcggcccagcctgcggctgccagaggacacaggttctgctgcggaacctgcagacatggccataacaggccacagtgctcgggcccacacagcctggacccacatggccctgtgtcacctcctcaggggcaggcttcagggcctcgaccctagaggctgcccctcggttctgctccatggacggcgcaggcaggcccaggcctgtgacgagttcacggaagctccaggatgacccccgctctgcgccctcctccagcattccagaccacaaaccactctgggctaaaacgaggcatcgccagagcatcccacttcctcggaaagctgcggtctggggacgcgtcttggccctgaagaggctccagatggctcccatcaggcctctccgcctacgtgcggccgacatggagtgacagagcgtcggggacacagaattcagagctgggcctggggctgctttgagatactgatggctgccagggggcacagagacccgtcctgcagacagggctgtgagggccacagggggcctcggggagaggcagtgggagggaggacagtgggggcctccagctgggtgagcagctggagcgaggggggcccggggcttgtgatggtgctgccgaccctagaggtgccggccccacgatggagagcacgtagtgccccccgggagtcaggaggccgggcctgacctcgggggctgcagccaggggaggccggcaccccagataacccccaaagaactgcaggccctgaggcgaggccagagtgggggcgggggcaggtcccagccgaggaggtgctccgtgctgcctcagcagaacccatgatgggctggcccaaggctctgaaggtggaaaggcctcacacattctgccccggctgacgccttccttgggccagtgctcgggggtgtgtaacaaacgccaagacgcattgtaaagaaggaagcctgcgtttccatcaccggcttaatatcaaacaaaagtgcaattttgaaaatgtagtccaaggttttctgtggtgcggaaatggccaggccagacctccgtgggtggtccttcgtgtccacgtcagcgccctacatccacactgtgggcaccatgacctcacatgcggagcggagcagggccggcgcccggagagccaggctggtcacgaacgaggcctagagggcgtcaggccccaaagcactcacaggcttctcctctgtcctcggggccttcagacacctgcatgcgccgattcagccacccgcgcgcgccgattcccctggccatggggtttccaaagtgtgtgctcagaggacagtttcctccaggatgacctgtcagtggctctctgtgccggggacgtcgcgtgctgggtcccggtctgaatgcttcctaacgatttacccagttccttttctccactcaggaggcgtttgctgagaggcacaggctgagcccccgtgctgatgccacgaccgagggaacgggtctccctgtcggcgtgaactgacccggccaggcgtccactgccactcggactgtctcccaggcacgtggcgcccacacgggcagaacacgccctccacacacgcggcttcgggcagaacacgaggcgccctccacacacgcggcttcggggcttgtcatgaaaaaagctgaatgctgggggtgcagctttcaccaacagaatcccgtttggaagggacgcggtgagacatgatccaccctaagttgtgatcctgggtgagccgccgtccacaccctgctgagggtcccttcacccactttattctccagaaaaccctgcccatcagggctgagtcccacgccttccctctccgtccaggcctggctttgacctctggggtcgtgtggggcacaggggacaccctatccaggcagaggccctacggctatctggaggaagtggtgggagctgggcttctgcctggaggatgcacccagaggggtcacagtccacacagagacacacgggtgccttccagatggctgagccagtccagcccagaagggcctgggggttgggggctgcacctggcctgtccccaccagcagggctcagggcttcccaaggtgtgtgggggacggggcagcacctctcaaccaggtcacctgaaacccgaactgaaaggcatcctaagttaagacattaactcccattgtcaaggtgccatcgtcaattctgtctccaaatccttctttgttatttcatgtattcacagagtgacgctccgtgtttcgttcagcctgcaggcctgcagaagctgcatctcgggatggccaagagcccggccaggccccacggctgcacccaggacgggattcatgccccatgcctggcttctcacgaccacagagtgcctttcccgggactggatggaggcagagtgagagaagagcctggagcaagtgttttggaccacagtgatcaaacacggagcccgtggg

SEQ ID NO: 254 - DNA, 700 bp, Homo sapiens
  source 1..700: mol_type = other DNA
aagaaaggccagaccgggcacggtggctcacgcctgtaatcccaacacttggggaggccgaggcgggcagatcacctgaggtcaggagttcgagaccagcctggccaacagggtgaaaccccgtctctactaaaaatacaaaaaaaaattagccgggcgtggtggcaggcacctgtaatcccagctaatcgggaggctgaggcaggagaaaatcacttgaacctgggaggcggaggctgcagtgagctgagatcgcgccactgcactccagcctgggtgagggagcgagactgtctcaaaaaaaaaaaaaaaaaaaaaaaaaaaggaaagaaaggcccggtgagatgctttctcttaaacacggccctgcacgttgagttgctgcctcctgtggcctatttcacgtttatgcaaagtcgggcgcctgatgcggggctcacccgccacaagcaggggtcctggtgctgctcatggaaggggccctacccagcccgcggggcactggctgggacggggctgcccaggtccgcccaggatccaaacacccagccccgcccagcggcccttcctggcctgcagtggaggctgtaatgggcaggggtggtgggaatcccagctcacagggcgcctgctcttagaagggcggcatctgggtccagaggtcagaaacgtcagatgcccatcccagaagtggcgggga

SEQ ID NO: 255 - DNA, 10000 bp, Homo sapiens
  source 1..10000: mol_type = other DNA
gggtgaatgagtagatgtatgggtgagtaggtgggtaggtgggtagatggatgggtgggtgggcgagtgtgtggttagatgatggatggctgaatggatgagtggggggatggatgggtgagtgggtgtatgtatggatgggttagtgggtgggtggatgaatggatgggtgcataaaggatggatggatgaatgagttagtgggttggcagatggatggatgggtgagtcagtggatagatggatgggtgggtggatagaggatggatggttgggtaggtgatgggtggatgagtggatagatgggtatgtgagtgagtggggggatgggtaggtgggtggatggatggttaggtgaatgagtggatggacagacggacagtgggtggatggatgagtgaacggatggaccgatggatgaatgggtgggtgggtagaggatggacggacaggtgagtgggtgggtggatggatagatgggtaagtgagtggatagatagatgggtgggtggacagaggatgggtggatgaatggatgggttagtgggtggctgggtggatggatgatggatgggtgactgggtggatggatggatgggttagtgggtggctgggtggatagatggatgggtgattgggcgaatgggcgaatgggtggatgggtgggcgtggagttggtgggtacatgataatggggtggaatacccatggattggaatgagctgttttggctgctatttctgggacacccagctctgccaggcccctacccctctggtgggccaggctctgacggtggccactcatggcctttctagctctggtgccagcatagggaaggaggaggcacagccttgtcttactccttgcacctgttagcccccccccccgccaagggaggacccgtggttggggacagcacagggggccctgctgtgtgcagggactgtccctggggccactgaagcccacctgttcttgttccttctcaggcggatcctggtccccctggtgagccaggccctcgggggccaagaggagtcccaggacccgaggtaggttggtggccagtccccatgccctccccccaacctgccaggccaacacacacccaagcctcgtggttctgcccacggtggacccacgtatcagtgggcagtggcctgggagagactcagccacccagccttggccccagagtctcagcctcatccttccttccccagggtgagcccggcccccctggagaccccggtctcacggtaggtgtcacatggggcagaaccagtgtccttctcctgccaaaactagacaccaagagcagcaggggtgggggaaggtcagctggcacggtcagagagcaagatcagtggaggaggtcagagggcaaggtcagagagcaagcttggttggggaaggtcacagggcaaggttggtggggggaggagggtggcagcgaggttggtagggacaggacccgccagcctccccgcatggctgcctccacacgtgggctggaatgtcccgggacccccaggccaggaccttgctgtggaaactcttctggggccccggggggactaccctgcctgccgtgtgcattgcaggagtgtgacgtcatgacctacgtgagggagacctgcgggtgctgcggtgaggcactgcccacggcagggtcggggcccatgcaccgggtggagggcgggagtgcagcagggctgggtcatcgctgggtcctgcatgtgcacgtgaccctagggtctgaggtctccccggtaccccccgatgaccctgccacccccccagactgtgagaagcgctgtggcgccctggacgtggtcttcgtcatcgacagctccgagagcattgggtacaccaacttcacactggagaagaacttcgtcatcaacgtggtcaacaggctgggtgccatcgctaaggaccccaagtccgagacaggtcagcggggcaggggcgggtgcagcattgcggggggccgggcggggcgtgggaggcgatgagatgggagaagtccagacgcgtccctccaacgagggcctctgcatggctggggatgccccagaccccgaggcctctggcaacgacctcacgcgtgcggcttgcagggacgcgtgtgggcgtggtgcagtacagccacgagggcacctttgaggccatccagctggacgacgaacgtatcgactccctgtcgagcttcaaggaggctgtcaagaacctcgagtggattgcgggcggcacctggacaccctcagccctcaagtttgcctacgaccgcctcatcaaggagagccggcgccagaagacacgtgtgtttgcggtggtcatcacggacgggcgccacgaccctcgggacgatgacctcaacttgcgggcgctgtgcgaccgcgacgtcacagtgacggccatcggcatcggggacatgttccacgagaagcacgagagtgaaaacctctactccatcgcctgcgacaagccacagcaggtgcgcaacatgacgctgttctccgacctggtcgctgagaagttcatcgatgacatggaggacgtcctctgcccgggtgagcgtgtgggcgcggggcagtcggccgaggagcagcaggccccagccgctgtctagcgtgagccccagggacacccctcacctgagggatgaatgtgcagcccaggatcttgggctgtgggtgggaaggggtcgggccctctcggggctgcagggcagaggccagctgcaccctgagcctgtctaggcagatcagtgaacggccgctgagggttcgctagggactgaccctggcctggcccggcctctctcctctcttccagaccctcagatcgtgtgcccagaccttccctgccaaacaggtaatgcagggcaccctgagccaccaccccagactagcaaagcagccctggtgtccttcctcctcgagggccgggctgggggaggggccgtgcagggacccggggggcggcggagccactgcggaggctgctccttagggagatggccccaggatggcagcacaggggaggaggggcttggggaaggcaggctcccaggaacgcaggaacagcatcacgaggccatgaggtgggtgctgctagcctggcgctgtgctcggcatgtggccactggtcttgaaggcccaccatgggccttgcagtctccctcagctgccgcccagctcccatgggctggccgtgcatgtgccactcggaggaagccctggattcagtgagtgaaaccatcccggggtggaagcactgacaccccccagcaccagcaggtcttgctccaaccctggcctgcctcggagctgcagctgcggctctcacatctctgggagtgggggagcccatgtcccggatgtggcccacgtgggtgtgaagctggagctgggggtgccgtccaggctctgctggacgtggtgctgcccccatggtgcactgctgcaccgtacctgggcccacaggaggtccccgggggcgttaggagctgagtccccctcagtgagccgtcccctccaggagtgtgagggtagggatgccatggagacagggtgggagggtccgacctggaggaccacagggaggaaacctcagggtctgcggtacgaagtcagcgcttcctcagcacgcgggtcgcggtgtgcgttcgggcgttccatggggagctcccggtgggtgagctgggccactgagcacattcacaggccctgaggctgccccaggggaggagccgtggactcagagccgaggttccccatacgtgctgcgacagagaacctagggcttgcacctgggtctggctgcccttcagcaggcgggcagcctctggccccacaacagtgggctgtgcttctgccgccaaggtgcaggcgtcctcccccagggtccacatcagcagcaggggcacctggaccctgagggcaggaaccagaccttggctcctccacccaccccctcgttcctgatggggcagggaagtctcgggaccccatgatgggcgacatggcgatggtcactgtgggtgctttgctatcaggtggggggccttcctctccactctgggtccagtgtgagtggccgctatggcttcccctccactccaggttctatcgtgagtgggtgggtgctgcgtctgtggatgtcacgtgacctttcctctttagcctatcattgtagttgggagttagttagcccgttgagcgtcattgaatttccagtgttgagccagccctgcgtgcccgggataaacccacctggccgtggtgtgtggccctgtttatgcacgtgggccctgattcgctgatgcctgcctgagggtttgcgcttatcggcgacatcagcctgcacttttcttttctcgtgatctctctggttctggcctcagggtgacgtgggcctcgtagggtcctgtggtggctcctccccagacggtgacatggagtgagcccattctccctcctgggagtgggtcactcaggccaccagagcaccacagggaaagcagccagggaggacacggaggcccttgaagctctggcctcttctgaggcctccaggacctgacagtgagtgggagcagccctggcagaacccctcccctcctctcggccgccctgacacctcatccccgacactcagagctcatcctccttcccagctgtttccaatttcaaagtgaactcgaccttgtggctccaggagatgcagcagggacagtgttaaatcggctttcaccagcccacacggccaggcatcctcctcggccctcctgggcactgggtggacaccactggctgtggcctggccctggccttctccagacagccctgtccaccccaaagcccagccaccctgggcctgcagcaggcctgtggagttctcagttgcgtggggaccagagggtgctggagaaacaaaccagacgcagctgaaggcagtcagggcagggcgcaatcagcgataagagctgcataggggccacagcgtaacctgagctccagtcggtggaaagaaaaggcagagacgttgcagaggccaggtctgctcaggggaagacagttctgggtgtagaggactcacatcccagagaggctgaggaagggtttaccaccgcaagctttctcaggcgggctcttgaggggtggctggggtcttcctggcgacgggcctgcggcactggaagccctactggagtttggcctgtctccggcacaggtttggacggagctgttttgtgctgaaaggttttctcggggtccgtggtgtcccccaaaggtgccaccgtgcgggtctcctagctccctgccagcttcctgtccctgtgctcactgcccccacgcctcctgccaaggccgagccacacacccgctccacctgcatttcctctaccgactcgccagcccaaatgccgctcttcactctggcctcgctgagcggctgcccgaggaggagctctaggccgacgcccaccgcaggccttacagtcttctctggacgctcccttgcagatgcaccgtggcctggcggcgagcccccggtcaccttcctccgcacggaagaggggccggacgccaccttccccaggaccattcccctgatccaacagttgctaaacgccacggagctcacgcaggacccggccgcctactcccagctggtggccgtgctggtctacaccgccgagcgggccaagttcgccaccggggtagagcggcaggactggatggagctgttcattgacacctttaagctggtgcacagggacatcgtgggggaccccgagaccgcgctggccctctgctaaagcccgggcacccgcccagccgggctgggccctccctgccacactagcttcccagggctgcccccgacaggctggctctcagtggaggccagagatctggaatcggggtcagcggggctacagtccttccaggggctctggggcagctcccagcctcttcccatgctggtggccaccgtgtcccttgctgcggctgcatcttccagtctctcctccgtcttcctgtggccgctctctttataagaaccctggtcattgaatttaaggcccaccccaagtccagaatgacctcgcaagacccttaactcactcccgtctgcagagtccttctttgctgcatcaggtcaccctcacaggctccagggtttgggtgtggaagtctttggaggcccttacttagcggcccagctgggctgccgtgcgtctgggatggggctgagggagggtgctgcccaggtgctggaggatgttccagcaccaggttccagcggagcctcggaaacaggccccagaggctggtgagcctcgctgggtgtgggcactaatcccgtgcatggtgactcgtgggcgctcacggcccacctggtggcaggtgaaggcttccggttgggcagcagatagtcctgggggaagctggcagtcctggcaccatgacgtatctgggctggtgtcatgcacagtagggcgaatggccacagctgcctgccagcagccctgatcccggggtgtctgcacccttccagcccaacctctgggtctccaaaagcacagtcgggggagcatccaccaggcacaacctctgcggtcctcagaggactgagcagagaatcccagggtccacaatgttggggagcggcagggatcaccatccaaagggagcggcccccacggcgagctgaccccgacgttctgactgcaggagccctcatccaggctgggctcctgccgggcacggctgtgaccatttctcagggccaggttctcgtccccacacccactgcacagggcaggccaggctggtcttcccactgtggggatgaaggatcctccacaggaggaggagagcagagtccacagacatcccaacagcctcagcctccctgtgcctggccggcccccacagcttccccgtctcctccaggccccacagacactgatgaatggacagagacccccaaaaccagctgccccttgcatgtctgtctccatatgtttggtgacagcagtgaaaatgttattagttttgagggggtttgggaagcccagcggtacctgaggagtttctggacatttaagccggttcctaggtgtggccttaacagggaggctgcccttcctttcactgaatgagctgcgtcactcataagctcactgagggaaccccatctgccagctcgtgcgtgctcagacggcgtccatgtctcaagcgttctgtgaaggctgcggtgcagcgtgaggtcaccctgctgtgttcagagctttgctcactgcctgcggggctggaccgttgcacctccagggcccccagaaaccgagtttcgggtcagggtcctctgtgtgcattcctgggggtccatgtaccagctgtgacgacgtccaggggttgggctgagaagcagacacccttggggaaactggctctgtccctcccctcccccatcccaggagctgaggtcttggtgaggccacagggccaggtccacgcaaggactgtccgtgtcctgtcctgtggtctctggccccacgtgacacccacacgtgtggtaggcagcctggcctgggttgtggctatggccaggcccccaagctgtccccgatgcccagggctggtgaccacccaggcaggtgggggccccacttggtaacagagtcatagggcagaacccacctgggctgccacagaaggtctggctgcccctgtgcccactgctccccaccatggccaatcagaagagtcaggggctcctggtctttccgggagggacgtggcccagccagctctaggtgttctgagcagctctgggacccagcgattgaggggtcaggctgggggtgtcagagccagggtcctccttaagtacctcccacactacacagacagtggcccttttgtgggcagcaaattcttgagccatgaaaggatgctttgggccccttccctcccaggagggcagcctgtgcagggatggtgctcagcaggtggacagggcctggggcctgtgtcagggtctcaggcctgggagcaccagcagaggagatggcggctcccagcagtgccgcctgaaagtgtcttgggctaaggacccacacccagggctgccctgcagaaacgcccccgcagagcccagtggtctgtgaggttgcaggcagggtgcgaatggaagggcacaggtgcggggctggcacctgcccggtcctgcccacctcccctccgcccagcccgcacctgcgtctccccacagagctgtccgtggcacagtgcacgcagcggcccgtggacatcgtcttcctgctggacggctccgagcggctgggtgagcagaacttccacaaggcccggcgcttcgtggagcaggtggcgcggcggctgacgctggcccggagggacgacgaccctctcaacgcacgcgtggcgctgctgcagtttggtggccccggcgagcagcaggtggccttcccgctgagccacaacctcacggccatccacgaggcgctggagaccacacaatacctgaactccttctcgcacgtgggcgcaggcgtggtgcacgccatcaatgccatcgtgcgcagcccgcgtggcggggcccggaggcacgcagagctgtccttcgtgttcctcacggacggcgtcacgggcaacgacagtctgcacgagtcggcgcactccatgcgcaagcagaacgtggtacccaccgtgctggccttgggcagcgacgtggacatggacgtgctcaccacgctcagcctgggtgaccgcgccgccgtgttccacgagaaggactatgacagcctggcgcaacccggcttcttcgaccgcttcatccgctggatctgctagcgccgccgcccgggccccgcagtcgagggtcgtgagcccaccccgtccatggtgctaagcgggcccgggtcccacacggccagcaccgctgctcactcggacgacgccctgggcctgcacctctccagctcctcccacggggtccccgtagccccggcccccgcccagccccaggtctccccaggccctccgcaggctgcccggcctccctccccctgcagccatcccaaggctcctgacctacctggcccctgagctctggagcaagccctgacccaataaaggctttgaacccattgcgtgcctgcttgcgagcttctgtgcgcaggagagacctcaaaggtgtcttgtggccaggagggaaacactgcagctgtcgctcgcccaccagggtcaatggctcccccgggcccagccctgacctcctaggacatcaactgcaggtgctggctgaccccgcctgtgcagaccccacagccttgatcagcaaactctccctccagccccagccaggcccaaagtgctctaagaagtgtcaccatggctgagggtcttctgtgggtggacgcatgattaacactagacggggagacagcaggtgctgagcctgttgtgttctgtgtggagatctcagtgagtttttgctgttcagaccccagggtccttcaggctcagctcaggagccccacagtgaaccagaggctccacaggcaggtgctgacctgacaggagtgggcttggtggccatcacagggcaccacagacacagcttgaacaactaccagtatcggccacaggcctggaggcatcagccgggccatgcttcctctggagggctagaggaggactagagaagggcctgccccggcctctccccagcatcccagggttcctgatctcctggataaggatacaagtcaccacactggactggggctcagcctgctctagaatacctcacctaagtcacagtggaccaggctcagcctgctctaaggtgagcttacccgagacactggaccagagatcagcctatcctgggataagctcacccgagtcacactggaccagggctcagcctattccgggatgagctcacccgagtc

SEQ ID NO: 256 - DNA, 800 bp, Homo sapiens
  source 1..800: mol_type = other DNA
gacacttccatgactgcagctgaccagtccacctgccagcggttgaccactcccacttcgccagcgaccgaaggggaggggaggggcctcacctgagggcaacagcagaacccaccacctggtcttgctttactcagacctgagggtgtgaaaggtgcccgtgacctcccgcatcagggagctggccgccaccctcgactcccggggagcaggcgtcccgcgaccccctcatctaccaggccatctgagctgggcggcgcctcacctccgctcccgggggagccggcctcagggtaggcatgcgccctgggtgggagcaggtcgtggccgccgccctcctggcagctctggctgagcagccgccgcagcatctgattctccttcaggaggcgcacctgcttcttcaggtccgcgttctcgctcaggagccggctcatcagctcgccgccttcagccatggcgggtgcgtccctccttgtccctcacggctcctgcagccccatggaggtgggagcccagagcccgcaggcaccacagaaacagcccaggcacggagttccgtagccaccaccgccttccacgccttgtgatgtcactgccctagtgatgaggtgcccagcaccctgcctgcccccgcgatggctcatggccccgttgaggcagtgaagctggaggcccgtggcgtgcacaggcagccactcccacattatgaccagggcccgagaatgccaaggacattaggcagctacgggatgtagcgactgtactccaagaggggcgtccaagccactccccattga

SEQ ID NO: 257 - DNA, 293 bp, Homo sapiens
  source 1..293: mol_type = other DNA
aggtggaggttgcagtgagccctcctcccctcctcccccttcccttcccacctcccatgcccccctttcttcctcccactcccctcccgaggccccgcttattctcccggcctgtggcggttcgtgcactcgctgagctcaggttctggtgaaggtgcccggagccgggtcccgccttcggcctgagctagagccgcgcgggcggccggcttcccccaaaccctgtgggaggggcatcccgaggaggcgaccccagagagtggggcgcggacaccttccctggggagggccag

SEQ ID NO: 258 - DNA, 474 bp, Homo sapiens
  source 1..474: mol_type = other DNA
ccttccagatgttccagaaggagaaggcggtgctggacgagctgggccgacgcacggggacccggctgcagcccctgacccggggcctcttcggagggagctgagggccgcgttccttctgaaagcgggacgcgggaggggtggaggctgcggggagccggggtcgcacacgaataaataacgaatgaacgtacgaggggaacctcctcttatttccttcacgttgcatcgggtatttttcgttattgtaaataaaacggttccgagccgtggcatcgagagggcgtctggagttcagggaacgcgtggcccccgcccgggagcaccgcgcagcgctcgcctctcgcccttcaagggggtccctgcccggagcctgcgcccccggagaggaaggggctcgaggggcttgggtgccgcagcgcgtccttccgtagaaaaggcttgcgtcagtatttcctgcttttacctcctgag

SEQ ID NO: 259 - DNA, 346 bp, Homo sapiens
  source 1..346: mol_type = other DNA
cagtatttcctgcttttacctcctgagtattggaatattcgagtaaaccctggagtttcagcgccagcgcacgcctcttcatcagggcagcgcgtcgcgagcgcgctggttccccggggcctcccggccacggacaccgctctagccagggccacggcgaggccgccgagcagcacctcagagacctgcgtgagttctaaagcctggggctactacaattctgctcatctgtttgtcctgtgaaatgattcagggacatgaaaatgccttcccactgacttgcgtcctgtcttagcctggacttgtccccttgggaacacgggccaggcccctctgttcctgaagt

SEQ ID NO: 260 - DNA, 490 bp, Homo sapiens
  source 1..490: mol_type = other DNA
atgtctgcagggaagaagcagggggaccctgaataaagtttccgtttttcctatttgttaaagtgatagagcattataggaccagagaacaggtgtgtctgtacactgtgcaggtccccggggcaggctctgagtccgtctgcacacggtgcgggtccccggggcgcgccctgagcccgtctgcacacggtgcgggtccccggggcgcgccctgagcccgtctgcacacggtgcgggtccccggggcgcgccctgagcccgtctgcacacggtgcgggtccccggggcgcgccctgagcccgtctgcacacggtgcgggtccccggggcgcgccctgagcccgtctgcacacggtgcgggtccccggggcgcgccctgagcccgtctgtacacggtgcgggtccccggggcgcgccctgagtctctactaaaaatacaaaaattagccaggcgtggtggttcaagcctgtaatcccagctccttgggagg

SEQ ID NO: 261 - DNA, 2000 bp, Homo sapiens
  source 1..2000: mol_type = other DNA
catacatggttattagaaaaggcatctcatccaaatgtggtggctcgtgcttgtaatcccagtgcttcaggaggccaagggaggaggattacttgagcctaagagtttgagaccagcctgggcaacacaacaagaccttgcctctacaaaaaacttaaaaactagctgggtatgatggtgcacacctgtagtcccagctacttgggaggcggaggcgggcagatcgcctgaggtcaggagttcgagaccagcctggccaacatgatgaaaccccgtctctactaaaaatacaaaaattagccgagtgtggtggtgcatgcctgtaatcccagctactcaggaggctgaggcaggagaatcacttgaacccgggaggcggaggttgccatgagccgagatcacgtcactgcactccagcctgggtgacagagcacaaaagacaggcatgactttgtacttaactgctcagctttgtaatcactgggggcccagatgctcacttggattctaactttgttggcatctgggcctaaaagccgtgatgcaggtgagcaatgatgcagagggctctgtgcgcctggcgggctctgtttgcctgctgggctctgtgcgcctgctgggctctgtgcgcccgggaaggtgcggccaccctcacgcggaaggcggccagcggatcccggtgcgcgcagctcccagcgctggggttccagcgccccgcctcttcctatagcaaccagcgggacctgccgtcccccggggcaccccgaggggtctgcgcccgcttctttccgaaacgggaaggcgctgggggctcggcagccagagggacgggttcagggagcgtccggtgagcctaagacgcgcctttgccggggttgccgggtgtctgcctctcacttaggtattaggaaccgtggcacaaatctgtaggttttcctctgggggtgggcggaggctccaaaccggacggttttctcctggaggactgtgttcagacagatactggtttccttatccgcaggtgtgcgcggcgctcgcaagtggtcagcataacgccgggcgaattcggaaagcccgtgcgtccgtggacgacccacttggaaggagttgggagaagtccttgttcccacgcgcggacgcttccctccgtgtgtccttcgagccacaaaaagcccagaccctaacccgctcctttctcccgccgcgtccatgcagaactccgccgttcctgggaggggaagcccgcgaggcgtcgggagaggcacgtcctccgtgagcaaagagctcctccgagcgcgcggcggggacgctgggccgacaggggaccgcgggggcagggcggagaggacccgccctcgagtcggcccagccctaacactcaggaccgcctccagccggaggtctgcgcccttctgaggaccctgcctgggggagcttattgcggttcttttgcaaatacccgctgcgcttggacggaggaagcgcccacgcgtcgaccccggaaacgaaggcctccctgatgggaacgcatgcgtccaggagcctttatttactcttaattctgcccgatgcttgtacgtgtgtgaaatgcttcagatgcttttgggagcgaggtgttacataaatcatggaaatgcctcctggtctcaccacacccagggtgacagctgagatgcggcttctccagggtggagcctcctcgttttccagagctgcttgttgaagtcttcccagggcccctgacttgcactggaaactgctcaccttggcatcgggatgtggagcaagaaatgcttttgttttcattcatcctagtgttcataaaatggaaaacaaataaggacatacaaaaacattaataaaataaattaatggaactagatttttcagaaagcacaacaaacacaaaatccaagtattgccatgtcagcaacacattcctactttaagttttatgaagttaattggagtagtggagaacaaaagtggatgtggggcag

SEQ ID NO: 262 - DNA, 88 bp, Homo sapiens
  source 1..88: mol_type = other DNA
gattgacagtttctccttccccagactggccaatcacaggcaggaagatgaaggttctgtgggctgcgttgctggtcacattcctggc

SEQ ID NO: 263 - DNA, 30 bp, synthetic construct
  misc_feature 1..30: note = Description of Artificial Sequence: Synthetic primer
  source 1..30: mol_type = other DNA
acgttggatgttgacagtttctccttcccc

SEQ ID NO: 264 - DNA, 30 bp, synthetic construct
  misc_feature 1..30: note = Description of Artificial Sequence: Synthetic primer
  source 1..30: mol_type = other DNA
acgttggatggaatgtgaccagcaacgcag

SEQ ID NO: 265 - DNA, 18 bp, synthetic construct
  misc_feature 1..18: note = Description of Artificial Sequence: Synthetic primer
  source 1..18: mol_type = other DNA
gcaggaagatgaaggtty

SEQ ID NO: 266 - DNA, 88 bp, synthetic construct
  misc_feature 1..88: note = Description of Artificial Sequence: Synthetic oligonucleotide
  source 1..88: mol_type = other DNA
gattgacagtttctccttccccagactggccaatcacaggcaggaagatgaaggttttgtgggctgcgttgctggtcacattcctggc

SEQ ID NO: 267 - DNA, 90 bp, Homo sapiens
  source 1..90: mol_type = other DNA
gagttttggatagtaaaataagtttcgaactctggcacctttcaattttgtcgcactctccttgtttttgacaatgcaatcatatgcttc

SEQ ID NO: 268 - DNA, 31 bp, synthetic construct
  misc_feature 1..31: note = Description of Artificial Sequence: Synthetic primer
  source 1..31: mol_type = other DNA
acgtggatagtaaaataagtttcgaactctg

SEQ ID NO: 269 - DNA, 27 bp, synthetic construct
  misc_feature 1..27: note = Description of Artificial Sequence: Synthetic primer
  source 1..27: mol_type = other DNA
gaagcatatgattgcattgtcaaaaac

SEQ ID NO: 270 - DNA, 20 bp, synthetic construct
  misc_feature 1..20: note = Description of Artificial Sequence: Synthetic primer
  source 1..20: mol_type = other DNA
atttcaattttgtcgcacty

SEQ ID NO: 271 - DNA, 90 bp, synthetic construct
  misc_feature 1..90: note = Description of Artificial Sequence: Synthetic oligonucleotide
  source 1..90: mol_type = other DNA
gagttttggatagtaaaataagtttcgaactctggcacctttcaattttgtcgcactttccttgtttttgacaatgcaatcatatgcttc

SEQ ID NO: 272 - DNA, 97 bp, Homo sapiens
  source 1..97: mol_type = other DNA
gaactcctctttgtctctgcgtgcccggcgcgcccccctcccggtgggtgataaacccactctggcgccggccatgcgctgggtgattaatttgcga

SEQ ID NO: 273 - DNA, 29 bp, synthetic construct
  misc_feature 1..29: note = Description of Artificial Sequence: Synthetic primer
  source 1..29: mol_type = other DNA
acgttggatgtctttgtctctgcgtgccc

SEQ ID NO: 274 - DNA, 30 bp, synthetic construct
  misc_feature 1..30: note = Description of Artificial Sequence: Synthetic primer
  source 1..30: mol_type = other DNA
acgttggatgttaatcacccagcgcatggc

SEQ ID NO: 275 - DNA, 22 bp, synthetic construct
  misc_feature 1..22: note = Description of Artificial Sequence: Synthetic primer
  source 1..22: mol_type = other DNA
cccctcccggtgggtgataaay

SEQ ID NO: 276 - DNA, 97 bp, synthetic construct
  misc_feature 1..97: note = Description of Artificial Sequence: Synthetic oligonucleotide
  source 1..97: mol_type = other DNA
gaactcctctttgtctctgcgtgcccggcgcgcccccctcccggtgggtgataaatccactctggcgccggccatgcgctgggtgattaatttgcga

SEQ ID NO: 277 - DNA, 86 bp, Homo sapiens
  source 1..86: mol_type = other DNA
ccattggccgtccgccgtggcagtgcgggcgggagcgcagggagagaaccacagctggaatccgattcccaccccaaaacccagga

SEQ ID NO: 278 - DNA, 29 bp, synthetic construct
  misc_feature 1..29: note = Description of Artificial Sequence: Synthetic primer
  source 1..29: mol_type = other DNA
acgttggatgccattggccgtccgccgtg

SEQ ID NO: 279 - DNA, 31 bp, synthetic construct
  misc_feature 1..31: note = Description of Artificial Sequence: Synthetic primer
  source 1..31: mol_type = other DNA
acgttggatgtcctgggttttggggtgggaa

SEQ ID NO: 280 - DNA, 19 bp, synthetic construct
  misc_feature 1..19: note = Description of Artificial Sequence: Synthetic primer
  source 1..19: mol_type = other DNA
ttccagctgtggttctctc

SEQ ID NO: 281 - DNA, 86 bp, synthetic construct
  misc_feature 1..86: note = Description of Artificial Sequence: Synthetic oligonucleotide
  source 1..86: mol_type = other DNA
ccattggccgtccgccgtggcagtgcgggcgggagcgcagagagagaaccacagctggaatccgattcccaccccaaaacccagga

SEQ ID NO: 282 - DNA, 31 bp, synthetic construct
  misc_feature 1..31: note = Description of Artificial Sequence: Synthetic primer
  source 1..31: mol_type = other DNA
acgttggatgacatggtcggccccacggaat

SEQ ID NO: 283 - DNA, 30 bp, synthetic construct
  misc_feature 1..30: note = Description of Artificial Sequence: Synthetic primer
  source 1..30: mol_type = other DNA
acgttggatgacccattggccgtccgccgt

SEQ ID NO: 284 - DNA, 31 bp, synthetic construct
  misc_feature 1..31: note = Description of Artificial Sequence: Synthetic primer
  source 1..31: mol_type = other DNA
acgttggatggaactcctctttgtctctgcg

SEQ ID NO: 285 - DNA, 31 bp, synthetic construct
  misc_feature 1..31: note = Description of Artificial Sequence: Synthetic primer
  source 1..31: mol_type = other DNA
acgttggatgcgcagcaacgggaccgctaca

SEQ ID NO: 286 - DNA, 29 bp, synthetic construct
  misc_feature 1..29: note = Description of Artificial Sequence: Synthetic primer
  source 1..29: mol_type = other DNA
acgttgcgtagcaacctgttacatattaa

SEQ ID NO: 287 - DNA, 31 bp, synthetic construct
  misc_feature 1..31: note = Description of Artificial Sequence: Synthetic primer
  source 1..31: mol_type = other DNA
acgttggatgcatagaggcccatgatggtgg

SEQ ID NO: 288 - DNA, 33 bp, synthetic construct
  misc_feature 1..33: note = Description of Artificial Sequence: Synthetic primer
  source 1..33: mol_type = other DNA
acgttggatggtgtggtcagctcttcccttcat

SEQ ID NO: 289 - DNA, 32 bp, synthetic construct
  misc_feature 1..32: note = Description of Artificial Sequence: Synthetic primer
  source 1..32: mol_type = other DNA
acgttggatgctccttcctagtgtgagaaccg

SEQ ID NO: 290 - DNA, 31 bp, synthetic construct
  misc_feature 1..31: note = Description of Artificial Sequence: Synthetic primer
  source 1..31: mol_type = other DNA
acgttggatgttttggggtgggaatcggatt

SEQ ID NO: 291 - DNA, 29 bp, synthetic construct
  misc_feature 1..29: note = Description of Artificial Sequence: Synthetic primer
  source 1..29: mol_type = other DNA
acgttggatgtggcatggccggcgccaga

SEQ ID NO: 292 - DNA, 32 bp, synthetic construct
  misc_feature 1..32: note = Description of Artificial Sequence: Synthetic primer
  source 1..32: mol_type = other DNA
acgttggcatctaggtaggtctttgtagccaa

SEQ ID NO: 293 - DNA, 32 bp, synthetic construct
  misc_feature 1..32: note = Description of Artificial Sequence: Synthetic primer
  source 1..32: mol_type = other DNA
acgttggatctgagcaaaggcaatcaacaccc

SEQ ID NO: 294 - DNA, 32 bp, synthetic construct
  misc_feature 1..32: note = Description of Artificial Sequence: Synthetic primer
  source 1..32: mol_type = other DNA
acgttggatgaccttctgcccctctactccaa

SEQ ID NO: 295 - DNA, 33 bp, synthetic construct
  misc_feature 1..33: note = Description of Artificial Sequence: Synthetic primer
  source 1..33: mol_type = other DNA
acgttggcccacatgtaatgtgttgaaaaagca

SEQ ID NO: 296 - DNA, 18 bp, synthetic construct
  misc_feature 1..18: note = Description of Artificial Sequence: Synthetic primer
  source 1..18: mol_type = other DNA
caggttccggggcttggg

SEQ ID NO: 297 - DNA, 19 bp, synthetic construct
  misc_feature 1..19: note = Description of Artificial Sequence: Synthetic primer
  source 1..19: mol_type = other DNA
cgcagggagagaaccacag

SEQ ID NO: 298 - DNA, 21 bp, synthetic construct
  misc_feature 1..21: note = Description of Artificial Sequence: Synthetic primer
  source 1..21: mol_type = other DNA
cccctcccggtgggtgataaa

SEQ ID NO: 299 - DNA, 21 bp, synthetic construct
  misc_feature 1..21: note = Description of Artificial Sequence: Synthetic primer
  source 1..21: mol_type = other DNA
aaagctgtaggacaatcgggt

SEQ ID NO: 300 - DNA, 22 bp, synthetic construct
  misc_feature 1..22: note = Description of Artificial Sequence: Synthetic primer
  source 1..22: mol_type = other DNA
catttttctacatcctttgttt

SEQ ID NO: 301 - DNA, 23 bp, synthetic construct
  misc_feature 1..23: note = Description of Artificial Sequence: Synthetic primer
  source 1..23: mol_type = other DNA
agaagatcaccaggcagaagagg

SEQ ID NO: 302 - DNA, 25 bp, synthetic construct
  misc_feature 1..25: note = Description of Artificial Sequence: Synthetic primer
  source 1..25: mol_type = other DNA
acttggagaacaaaggacaccgtta

SEQ ID NO: 303 - DNA, 81 bp, synthetic construct
  misc_feature 1..81: note = Description of Artificial Sequence: Synthetic oligonucleotide
  source 1..81: mol_type = other DNA
ggtcggccccacggaatcccggctctgtgtgcgcccaggttccggggcttgggtgttgccggttctcacactaggaaggag

SEQ ID NO: 304 - DNA, 79 bp, synthetic construct
  misc_feature 1..79: note = Description of Artificial Sequence: Synthetic oligonucleotide
  source 1..79: mol_type = other DNA
ccattggccgtccgccgtggcagtgcgggcgggagcgcagagagagaaccacagctggaatccgattcccaccccaaaa

SEQ ID NO: 305 - DNA, 77 bp, synthetic construct
  misc_feature 1..77: note = Description of Artificial Sequence: Synthetic oligonucleotide
  source 1..77: mol_type = other DNA
gaactcctctttgtctctgcgtgcccggcgcgcccccctcccggtgggtgataaatccactctggcgccggccatgc

SEQ ID NO: 306 - DNA, 81 bp, synthetic construct
  misc_feature 1..81: note = Description of Artificial Sequence: Synthetic oligonucleotide
  source 1..81: mol_type = other DNA
gcagcaacgggaccgctacagccactggacaaagccgtaggacaatcgggtaacattggctacaaagacctacctagatgc

SEQ ID NO: 307 - DNA, 95 bp, synthetic construct
  misc_feature 1..95: note = Description of Artificial Sequence: Synthetic oligonucleotide
  source 1..95: mol_type = other DNA
gcgtagcaacctgttacatattaaagttttattatactacatttttctacatcctttgtttcagagtgttgattgcctttgctcagtatcttcag

SEQ ID NO: 308 - DNA, 86 bp, synthetic construct
  misc_feature 1..86: note = Description of Artificial Sequence: Synthetic oligonucleotide
  source 1..86: mol_type = other DNA
ccttctgcccctctactccaagcgctacaccctcttctgcctggtgatctttgccggcgtcctggccaccatcatgggcctctatg

SEQ ID NO: 309 - DNA, 83 bp, synthetic construct
  misc_feature 1..83: note = Description of Artificial Sequence: Synthetic oligonucleotide
  source 1..83: mol_type = other DNA
gtgtggtcagctcttcccttcatcacatacttggagaacaaaggacaccgttatccatgctttttcaacacattacatgtggg

SEQ ID NO: 310 - DNA, 30 bp, synthetic construct
  misc_feature 1..30: note = Description of Artificial Sequence: Synthetic primer
  source 1..30: mol_type = other DNA
acgttggatgttctgcccctctactccaag

SEQ ID NO: 311 - DNA, 30 bp, synthetic construct
  misc_feature 1..30: note = Description of Artificial Sequence: Synthetic primer
  source 1..30: mol_type = other DNA
acgttggatgtcagctcttcccttcatcac

SEQ ID NO: 312 - DNA, 30 bp, synthetic construct; identical to SEQ ID NO: 263
  misc_feature 1..30: note = Description of Artificial Sequence: Synthetic primer
  source 1..30: mol_type = other DNA

SEQ ID NO: 313 - DNA, 28 bp, synthetic construct
  misc_feature 1..28: note = Description of Artificial Sequence: Synthetic primer
  source 1..28: mol_type = other DNA
acgttggatgcggtcggccccacggaat

SEQ ID NO: 314 - DNA, 31 bp, synthetic construct; identical to SEQ ID NO: 268
  misc_feature 1..31: note = Description of Artificial Sequence: Synthetic primer
  source 1..31: mol_type = other DNA

SEQ ID NO: 315 - DNA, 30 bp, synthetic construct
  misc_feature 1..30: note = Description of Artificial Sequence: Synthetic primer
  source 1..30: mol_type = other DNA
acgttggatgcacagctcaccgcagcaacg

SEQ ID NO: 316 - DNA, 29 bp, synthetic construct
  misc_feature 1..29: note = Description of Artificial Sequence: Synthetic primer
  source 1..29: mol_type = other DNA
acgttggatgtctttgtctctgcgtgccc

SEQ ID NO: 317 - DNA, 29 bp, synthetic construct
  misc_feature 1..29: note = Description of Artificial Sequence: Synthetic primer
  source 1..29: mol_type = other DNA
acgttggatggactgagccccagaactcg

SEQ ID NO: 318 - DNA, 29 bp, synthetic construct
  misc_feature 1..29: note = Description of Artificial Sequence: Synthetic primer
  source 1..29: mol_type = other DNA
acgttggatgaagccaagtttccctccgc

SEQ ID NO: 319 - DNA, 30 bp, synthetic construct
  misc_feature 1..30: note = Description of Artificial Sequence: Synthetic primer
  source 1..30: mol_type = other DNA
acgttagcgtagcaacctgttacatattaa

SEQ ID NO: 320 - DNA, 30 bp, synthetic construct
  misc_feature 1..30: note = Description of Artificial Sequence: Synthetic primer
  source 1..30: mol_type = other DNA
acgttggatgcatagaggcccatgatggtg

SEQ ID NO: 321 - DNA, 31 bp, synthetic construct
  misc_feature 1..31: note = Description of Artificial Sequence: Synthetic primer
  source 1..31: mol_type = other DNA
acgttggatgcctacctcccacatgtaatgt

SEQ ID NO: 322 - DNA, 30 bp, synthetic construct; identical to SEQ ID NO: 264
  misc_feature 1..30: note = Description of Artificial Sequence: Synthetic primer
  source 1..30: mol_type = other DNA

SEQ ID NO: 323 - DNA, 32 bp, synthetic construct; identical to SEQ ID NO: 289
  misc_feature 1..32: note = Description of Artificial Sequence: Synthetic primer
  source 1..32: mol_type = other DNA

SEQ ID NO: 324 - DNA, 27 bp, synthetic construct
  misc_feature 1..27: note = Description of Artificial Sequence: Synthetic primer
  source 1..27: mol_type = other DNA
gaagcatatgattgcattgtcaaaaac

SEQ ID NO: 325 - DNA, 32 bp, synthetic construct
  misc_feature 1..32: note = Description of Artificial Sequence: Synthetic primer
  source 1..32: mol_type = other DNA
acgttggatgctaggtaggtctttgtagccaa

SEQ ID NO: 326 - DNA, 30 bp, synthetic construct; identical to SEQ ID NO: 274
  misc_feature 1..30: note = Description of Artificial Sequence: Synthetic primer
  source 1..30: mol_type = other DNA

SEQ ID NO: 327 - DNA, 30 bp, synthetic construct
  misc_feature 1..30: note = Description of Artificial Sequence: Synthetic primer
  source 1..30: mol_type = other DNA
acgttggatggtgggtttgtgctttccacg

SEQ ID NO: 328 - DNA, 30 bp, synthetic construct
  misc_feature 1..30: note = Description of Artificial Sequence: Synthetic primer
  source 1..30: mol_type = other DNA
acgttggatgcttttgctttcccagccagg

SEQ ID NO: 329 - DNA, 30 bp, synthetic construct
  misc_feature 1..30: note = Description of Artificial Sequence: Synthetic primer
  source 1..30: mol_type = other DNA
acgttggatgctgagcaaaggcaatcaaca

SEQ ID NO: 330 - DNA, 17 bp, synthetic construct
  misc_feature 1..17: note = Description of Artificial Sequence: Synthetic primer
  source 1..17: mol_type = other DNA
ttctgcctggtgatctt

SEQ ID NO: 331 - DNA, 17 bp, synthetic construct
  misc_feature 1..17: note = Description of Artificial Sequence: Synthetic primer
  source 1..17: mol_type = other DNA
aacaaaggacaccgtta

SEQ ID NO: 332 - DNA, 17 bp, synthetic construct
  misc_feature 1..17: note = Description of Artificial Sequence: Synthetic primer
  source 1..17: mol_type = other DNA
gcaggaagatgaaggtt

SEQ ID NO: 333 - DNA, 18 bp, synthetic construct
  misc_feature 1..18: note = Description of Artificial Sequence: Synthetic primer
  source 1..18: mol_type = other DNA
aaggttccggggcttggg

SEQ ID NO: 334 - DNA, 19 bp, synthetic construct
  misc_feature 1..19: note = Description of Artificial Sequence: Synthetic primer
  source 1..19: mol_type = other DNA
atttcaattttgtcgcact

SEQ ID NO: 335 - DNA, 19 bp, synthetic construct
  misc_feature 1..19: note = Description of Artificial Sequence: Synthetic primer
  source 1..19: mol_type = other DNA
agctgtaggacaatcgggt

SEQ ID NO: 336 - DNA, 20 bp, synthetic construct
  misc_feature 1..20: note = Description of Artificial Sequence: Synthetic primer
  source 1..20: mol_type = other DNA
ccctcccggtgggtgataaa

SEQ ID NO: 337 - DNA, 20 bp, synthetic construct
  misc_feature 1..20: note = Description of Artificial Sequence: Synthetic primer
  source 1..20: mol_type = other DNA
agggccggggtctgcgcgtg

SEQ ID NO: 338 - DNA, 21 bp, synthetic construct
  misc_feature 1..21: note = Description of Artificial Sequence: Synthetic primer
  source 1..21: mol_type = other DNA
gaggcactgcccggacaaacc

SEQ ID NO: 339 - DNA, 22 bp, synthetic construct
  misc_feature 1..22: note = Description of Artificial Sequence: Synthetic primer
  source 1..22: mol_type = other DNA
catttttctacatcctttgttt

SEQ ID NO: 340 - DNA, 86 bp, synthetic construct; identical to SEQ ID NO: 308
  misc_feature 1..86: note = Description of Artificial Sequence: Synthetic oligonucleotide
  source 1..86: mol_type = other DNA

SEQ ID NO: 341 - DNA, 90 bp, synthetic construct
  misc_feature 1..90: note = Description of Artificial Sequence: Synthetic oligonucleotide
  source 1..90: mol_type = other DNA
gtgtggtcagctcttcccttcatcacatacttggagaacaaaggacaccgttatccatgctttttcaacacattacatgtgggaggtagg

SEQ ID NO: 342 - DNA, 88 bp, synthetic construct; identical to SEQ ID NO: 266
  misc_feature 1..88: note = Description of Artificial Sequence: Synthetic oligonucleotide
  source 1..88: mol_type = other DNA

SEQ ID NO: 343 - DNA, 98 bp, synthetic construct
  misc_feature 1..98: note = Description of Artificial Sequence: Synthetic oligonucleotide
  source 1..98: mol_type = other DNA
aaaaccagagattcgcggtcggccccacggaatcccggctctgtgtgcgcccaggttccggggcttgggtgttgccggttctcacactaggaaggagc

SEQ ID NO: 344 - DNA, 90 bp, synthetic construct; identical to SEQ ID NO: 271
  misc_feature 1..90: note = Description of Artificial Sequence: Synthetic oligonucleotide
  source 1..90: mol_type = other DNA

SEQ ID NO: 345 - DNA, 95 bp, synthetic construct
  misc_feature 1..95: note = Description of Artificial Sequence: Synthetic oligonucleotide
  source 1..95: mol_type = other DNA
gcagccagctcaccgcagcaacgggaccgctacagccactggacaaagctgtaggacaatcgggtgacattggctacaaagacctacctagatgc

SEQ ID NO: 346 - DNA, 97 bp, synthetic construct; identical to SEQ ID NO: 276
  misc_feature 1..97: note = Description of Artificial Sequence: Synthetic oligonucleotide
  source 1..97: mol_type = other DNA

SEQ ID NO: 347 - DNA, 86 bp, synthetic construct
  misc_feature 1..86: note = Description of Artificial Sequence: Synthetic oligonucleotide
  source 1..86: mol_type = other DNA
gtgggtttgtgctttccacgcgtgcacacacacgcgcagaccccggcccttgccccgcctacctccccgagttctggggctcagtc

SEQ ID NO: 348 - DNA, 89 bp, synthetic construct
  misc_feature 1..89: note = Description of Artificial Sequence: Synthetic oligonucleotide
  source 1..89: mol_type = other DNA
gcgccagcttttgctttcccagccagggcgcggtgaggtttgtccgggcagtgcctcgagcaactgggaaggccaaggcggagggaaac

SEQ ID NO: 349 - DNA, 96 bp, synthetic construct
  misc_feature 1..96: note = Description of Artificial Sequence: Synthetic oligonucleotide
  source 1..96: mol_type = other DNA
gcgtagcaacctgttacatattaaagttttattatactacatttttctacatcctttgttttagggtgttgattgcctttgctcagtatcttcagc

SEQ ID NO: 350 - DNA, 27 bp, synthetic construct
  misc_feature 1..27: note = Description of Artificial Sequence: Synthetic primer
  misc_feature 1..27: note = Description of Combined DNA/RNA Molecule: Synthetic primer
  source 1..27: mol_type = other DNA
  misc_feature 5: note = RNA
  misc_feature order(1..4,6..27): note = DNA
tagctgcgtagcaacctgttacatatt

SEQ ID NO: 351 - DNA, 25 bp, synthetic construct
  misc_feature 1..25: note = Description of Artificial Sequence: Synthetic primer
  misc_feature 1..25: note = Description of Combined DNA/RNA Molecule: Synthetic primer
  source 1..25: mol_type = other DNA
  misc_feature 5: note = RNA
  misc_feature order(1..4,6..25): note = DNA
tagctcagtttctccttccccagac

SEQ ID NO: 352 - DNA, 25 bp, synthetic construct
  misc_feature 1..25: note = Description of Artificial Sequence: Synthetic primer
  misc_feature 1..25: note = Description of Combined DNA/RNA Molecule: Synthetic primer
  source 1..25: mol_type = other DNA
  misc_feature 5: note = RNA
  misc_feature order(1..4,6..25): note = DNA
tagctggtcagctcttcccttcatc

SEQ ID NO: 353 - DNA, 24 bp, synthetic construct
  misc_feature 1..24: note = Description of Artificial Sequence: Synthetic primer
  misc_feature 1..24: note = Description of Combined DNA/RNA Molecule: Synthetic primer
  source 1..24: mol_type = other DNA
  misc_feature 5: note = RNA
  misc_feature order(1..4,6..24): note = DNA
tagctagctggtgcggagggtggg

SEQ ID NO: 354 - DNA, 26 bp, synthetic construct
  misc_feature 1..26: note = Description of Artificial Sequence: Synthetic primer
  misc_feature 1..26: note = Description of Combined DNA/RNA Molecule: Synthetic primer
  source 1..26: mol_type = other DNA
  misc_feature 5: note = RNA
  misc_feature order(1..4,6..26): note = DNA
tagctggcctttgcaacaaggatcac

SEQ ID NO: 355 - DNA, 25 bp, synthetic construct
  misc_feature 1..25: note = Description of Artificial Sequence: Synthetic primer
  misc_feature 1..25: note = Description of Combined DNA/RNA Molecule: Synthetic primer
  source 1..25: mol_type = other DNA
  misc_feature 5: note = RNA
  misc_feature order(1..4,6..25): note = DNA
tagcttctggtgacccccgcgcttc

SEQ ID NO: 356 - DNA, 24 bp, synthetic construct
  misc_feature 1..24: note = Description of Artificial Sequence: Synthetic primer
  misc_feature 1..24: note = Description of Combined DNA/RNA Molecule: Synthetic primer
  source 1..24: mol_type = other DNA
  misc_feature 5: note = RNA
  misc_feature order(1..4,6..24): note = DNA
tagctccctccacatcccgccatc

SEQ ID NO: 357 - DNA, 24 bp, synthetic construct
  misc_feature 1..24: note = Description of Artificial Sequence: Synthetic primer
  misc_feature 1..24: note = Description of Combined DNA/RNA Molecule: Synthetic primer
  source 1..24: mol_type = other DNA
  misc_feature 5: note = RNA
  misc_feature order(1..4,6..24): note = DNA
tagctacggaatcccggctctgtg

SEQ ID NO: 358 - DNA, 24 bp, synthetic construct
  misc_feature 1..24: note = Description of Artificial Sequence: Synthetic primer
  misc_feature 1..24: note = Description of Combined DNA/RNA Molecule: Synthetic primer
  source 1..24: mol_type = other DNA
  misc_feature 5: note = RNA
  misc_feature order(1..4,6..24): note = DNA
tagctggccctgctggcggtcata

SEQ ID NO: 359 - DNA, 24 bp, synthetic construct
  misc_feature 1..24: note = Description of Artificial Sequence: Synthetic primer
  misc_feature 1..24: note = Description of Combined DNA/RNA Molecule: Synthetic primer
  source 1..24: mol_type = other DNA
  misc_feature 5: note = RNA
  misc_feature order(1..4,6..24): note = DNA
tagctagcaacgggaccgctacag

SEQ ID NO: 360 - DNA, 26 bp, synthetic construct
  misc_feature 1..26: note = Description of Artificial Sequence: Synthetic primer
  misc_feature 1..26: note = Description of Combined DNA/RNA Molecule: Synthetic primer
  source 1..26: mol_type = other DNA
  misc_feature 5: note = RNA
  misc_feature order(1..4,6..26): note = DNA
tagcttaagtttcgaactctggcacc

SEQ ID NO: 361 - DNA, 25 bp, synthetic construct
  misc_feature 1..25: note = Description of Artificial Sequence: Synthetic primer
  misc_feature 1..25: note = Description of Combined DNA/RNA Molecule: Synthetic primer
  source 1..25: mol_type = other DNA
  misc_feature 5: note = RNA
  misc_feature order(1..4,6..25): note = DNA
tagctctcctctttgtctctgcgtg

SEQ ID NO: 362 - DNA, 25 bp, synthetic construct
  misc_feature 1..25: note = Description of Artificial Sequence: Synthetic primer
  misc_feature 1..25: note = Description of Combined DNA/RNA Molecule: Synthetic primer
  source 1..25: mol_type = other DNA
  misc_feature 5: note = RNA
  misc_feature order(1..4,6..25): note = DNA
tagcttgatgcccgatgccgccctt

SEQ ID NO: 363 - DNA, 27 bp, synthetic construct
  misc_feature 1..27: note = Description of Artificial Sequence: Synthetic primer
  misc_feature 1..27: note = Description of Combined DNA/RNA Molecule: Synthetic primer
  source 1..27: mol_type = other DNA
  misc_feature 5: note = RNA
  misc_feature order(1..4,6..27): note = DNA
gatctatactgagcaaaggcaatcaac

SEQ ID NO: 364 - DNA, 25 bp, synthetic construct
  misc_feature 1..25: note = Description of Artificial Sequence: Synthetic primer
  misc_feature 1..25: note = Description of Combined DNA/RNA Molecule: Synthetic primer
  source 1..25: mol_type = other DNA
  misc_feature 5: note = RNA
  misc_feature order(1..4,6..25): note = DNA
gatctgaatgtgaccagcaacgcag

SEQ ID NO: 365 - DNA, 26 bp, synthetic construct
  misc_feature 1..26: note = Description of Artificial Sequence: Synthetic primer
  misc_feature 1..26: note = Description of Combined DNA/RNA Molecule: Synthetic primer
  source 1..26: mol_type = other DNA
  misc_feature 5: note = RNA
  misc_feature order(1..4,6..26): note = DNA
gatctcctcccacatgtaatgtgttg

SEQ ID NO: 366 - DNA, 26 bp, synthetic construct
  misc_feature 1..26: note = Description of Artificial Sequence: Synthetic primer
  misc_feature 1..26: note = Description of Combined DNA/RNA Molecule: Synthetic primer
  source 1..26: mol_type = other DNA
  misc_feature 5: note = RNA
  misc_feature order(1..4,6..26): note = DNA
gatctatgggggagatggccggtgga

SEQ ID NO: 367 - DNA, 26 bp, synthetic construct
  misc_feature 1..26: note = Description of Artificial Sequence: Synthetic primer
  misc_feature 1..26: note = Description of Combined DNA/RNA Molecule: Synthetic primer
  source 1..26: mol_type = other DNA
  misc_feature 5: note = RNA
  misc_feature order(1..4,6..26): note = DNA
gatctcgcaatactagaaaccagggc

SEQ ID NO: 368 - DNA, 24 bp, synthetic construct
  misc_feature 1..24: note = Description of Artificial Sequence: Synthetic primer
  misc_feature 1..24: note = Description of Combined DNA/RNA Molecule: Synthetic primer
  source 1..24: mol_type = other DNA
  misc_feature 5: note = RNA
  misc_feature order(1..4,6..24): note = DNA
gatctcatctctgggtgcgccttg

SEQ ID NO: 369 - DNA, 23 bp, synthetic construct
  misc_feature 1..23: note = Description of Artificial Sequence: Synthetic primer
  misc_feature 1..23: note = Description of Combined DNA/RNA Molecule: Synthetic primer
  source 1..23: mol_type = other DNA
  misc_feature 5: note = RNA
  misc_feature order(1..4,6..23): note = DNA
gatctcagccgcctgctccatcg

SEQ ID NO: 370 - DNA, 25 bp, synthetic construct
  misc_feature 1..25: note = Description of Artificial Sequence: Synthetic primer
  misc_feature 1..25: note = Description of Combined DNA/RNA Molecule: Synthetic primer
  source 1..25: mol_type = other DNA
  misc_feature 5: note = RNA
  misc_feature order(1..4,6..25): note = DNA
gatctccttcctagtgtgagaaccg

SEQ ID NO: 371 - DNA, 25 bp, synthetic construct
  misc_feature 1..25: note = Description of Artificial Sequence: Synthetic primer
  misc_feature 1..25: note = Description of Combined DNA/RNA Molecule: Synthetic primer
  source 1..25: mol_type = other DNA
  misc_feature 5: note = RNA
  misc_feature order(1..4,6..25): note = DNA
gatcttgctcagcacgagggcccca

SEQ ID NO: 372 - DNA, 26 bp, synthetic construct
  misc_feature 1..26: note = Description of Artificial Sequence: Synthetic primer
  misc_feature 1..26: note = Description of Combined DNA/RNA Molecule: Synthetic primer
  source 1..26: mol_type = other DNA
  misc_feature 5: note = RNA
  misc_feature order(1..4,6..26): note = DNA
gatcttctaggtaggtctttgtagcc

SEQ ID NO: 373 - DNA, 28 bp, synthetic construct
  misc_feature 1..28: note = Description of Artificial Sequence: Synthetic primer
  misc_feature 1..28: note = Description of Combined DNA/RNA Molecule: Synthetic primer
  source 1..28: mol_type = other DNA
  misc_feature 5: note = RNA
  misc_feature order(1..4,6..28): note = DNA
gatctgaagcatatgattgcattgtcaa

SEQ ID NO: 374 - DNA, 25 bp, synthetic construct
  misc_feature 1..25: note = Description of Artificial Sequence: Synthetic primer
  misc_feature 1..25: note = Description of Combined DNA/RNA Molecule: Synthetic primer
  source 1..25: mol_type = other DNA
  misc_feature 5: note = RNA
  misc_feature order(1..4,6..25): note = DNA
gatctttaatcacccagcgcatggc

SEQ ID NO: 375 - DNA, 26 bp, synthetic construct
  misc_feature 1..26: note = Description of Artificial Sequence: Synthetic primer
  misc_feature 1..26: note = Description of Combined DNA/RNA Molecule: Synthetic primer
  source 1..26: mol_type = other DNA
  misc_feature 5: note = RNA
  misc_feature order(1..4,6..26): note = DNA
gatctgtctgtgctgggtgtttttgc

SEQ ID NO: 376 - DNA, 58 bp, synthetic construct
  misc_feature 1..58: note = Description of Artificial Sequence: Synthetic oligonucleotide
  source 1..58: mol_type = other DNA
aatgatacggcgaccaccgagatctacactctttccctacacgacgctcttccgatct

SEQ ID NO: 377 - DNA, 19 bp, synthetic construct
  misc_feature 1..19: note = Description of Artificial Sequence: Synthetic oligonucleotide
  source 1..19: mol_type = other DNA
gctcttccgatctatagct

SEQ ID NO: 378 - DNA, 65 bp, synthetic construct
  misc_feature 1..65: note = Description of Artificial Sequence: Synthetic oligonucleotide
  source 1..65: mol_type = other DNA
gatcggaagagcacacgtctgaactccagtcacagtcaacaatctcgtatgccgtcttctgcttg

SEQ ID NO: 379 - DNA, 58 bp, synthetic construct; identical to SEQ ID NO: 376
  misc_feature 1..58: note = Description of Artificial Sequence: Synthetic oligonucleotide
  source 1..58: mol_type = other DNA

SEQ ID NO: 380 - DNA, 33 bp, synthetic construct
  misc_feature 1..33: note = Description of Artificial Sequence: Synthetic primer
  source 1..33: mol_type = other DNA
acactctttccctacacgacgctcttccgatct

SEQ ID NO: 381 - DNA, 63 bp, synthetic construct
  misc_feature 1..63: note = Description of Artificial Sequence: Synthetic oligonucleotide
  source 1..63: mol_type = other DNA
gatcggaagagcacacgtctgaactccagtcacagtcaaatctcgtatgccgtcttctgcttg

SEQ ID NO: 382 - DNA, 33 bp, synthetic construct
  misc_feature 1..33: note = Description of Artificial Sequence: Synthetic primer
  source 1..33: mol_type = other DNA
gatcggaagagcacacgtctgaactccagtcac

SEQ ID NO: 383 - DNA, 29 bp, synthetic construct
  misc_feature 1..29: note = Description of Artificial Sequence: Synthetic oligonucleotide
  source 1..29: mol_type = other DNA
tctttccctacacgacgctcttccgatct

SEQ ID NO: 384 - DNA, 34 bp, synthetic construct
  misc_feature 1..34: note = Description of Artificial Sequence: Synthetic oligonucleotide
  source 1..34: mol_type = other DNA
gtgactggagttcagacgtgtgctcttccgatct

SEQ ID NO: 385 - DNA, 49 bp, synthetic construct
  misc_feature 1..49: note = Description of Artificial Sequence: Synthetic primer
  source 1..49: mol_type = other DNA
aatgatacggcgaccaccgagatctacactctttccctacacgacgctc

SEQ ID NO: 386 - DNA, 50 bp, synthetic construct
  misc_feature 1..50: note = Description of Artificial Sequence: Synthetic primer
  source 1..50: mol_type = other DNA
caagcagaagacggcatacgagatttgactgtgactggagttcagacgtg

SEQ ID NO: 387 - DNA, 10 bp, synthetic construct
  misc_feature 1..10: note = Description of Artificial Sequence: Synthetic oligonucleotide
  source 1..10: mol_type = other DNA
cgcaaccact

SEQ ID NO: 388 - DNA, 10 bp, synthetic construct
  misc_feature 1..10: note = Description of Artificial Sequence: Synthetic oligonucleotide
  source 1..10: mol_type = other DNA
cgcgaccact

(19) United States
(12) Patent Application Publication
(10) Pub. No.: US 2025/0154593 A1
(43) Pub. Date: May 15, 2025

(54) Processes and Compositions for Methylation-Based Enrichment of Nucleic Acid From a Sample Useful for Non-Invasive Diagnosis of Disease

(71) Applicant: Sequenom, Inc., San Diego, CA (US)

(72) Inventors: John Allen Tynan, San Diego, CA (US); Grant Hogg, San Diego, CA (US)

(21) Appl. No.: 18/921,873

(22) Filed: Oct. 21, 2024

Related U.S. Application Data
(63) Continuation of application No. 17/726,809, filed on Apr. 22, 2022, now abandoned.
Application No. 17/726,809 is a continuation of application No. 15/261,457, filed on Sep. 9, 2016, now Pat. No. 11,332,791.
Application No. 15/261,457 is a continuation of application No. 13/940,162, filed on Jul. 11, 2013, now abandoned.
(60) Provisional application No. 61/671,628, filed on Jul. 13, 2012.
(60) Provisional application No. 61/721,929, filed on Nov. 2, 2012.

(51) Int. Cl. C12Q 1/6883; C12Q 1/6881; C12Q 1/6886

(52) U.S. Cl. CPC C12Q 1/6883; C12Q 1/6881; C12Q 1/6886; C12Q 2600/154; C12Q 2600/156; C12Q 2600/16

(57) ABSTRACT

Provided are compositions and processes that utilize genomic regions that are differentially methylated between a mother and her fetus to separate, isolate or enrich fetal nucleic acid from a maternal sample. The compositions and processes described herein are particularly useful for non-invasive prenatal diagnostics, including the detection of chromosomal aneuploidies.

### RELATED APPLICATIONS

This patent application is a continuation of U.S. patent application Ser. No. 17/726,809, filed on Apr. 22, 2022, which is a continuation of U.S. patent application Ser. No. 15/261,457, filed on Sep. 9, 2016, now issued as U.S. Pat. No. 11,332,791, which is a continuation of U.S. patent application Ser. No. 13/940,162, filed on Jul. 11, 2013, now abandoned, which claims the benefit of U.S. Provisional Patent Application No. 61/671,628 filed on Jul. 13, 2012, and U.S. Provisional Patent Application No. 61/721,929, filed on Nov. 2, 2012. The entire content of each of the foregoing applications is incorporated herein by reference, including all text, tables and drawings.

### SEQUENCE LISTING

The instant patent application contains a Sequence Listing that has been submitted in ASCII format via EFS-Web and is hereby incorporated by reference in its entirety. The ASCII copy, created on Sep. 3, 2013, is named SEQ-6022-UT2_SL.txt and is 437,311 bytes in size.

### FIELD

The technology in part relates to prenatal diagnostics and enrichment methods.

### BACKGROUND

Non-invasive prenatal testing is becoming a field of rapidly growing interest. Early detection of pregnancy-related conditions, including complications during pregnancy and genetic defects of the fetus is of crucial importance, as it allows early medical intervention necessary for the safety of both the mother and the fetus. Prenatal diagnosis has been conducted using cells isolated from the fetus through procedures such as chorionic villus sampling (CVS) or amniocentesis. However, these conventional methods are invasive and present an appreciable risk to both the mother and the fetus. The National Health Service currently cites a miscarriage rate of between 1 and 2 percent following the invasive amniocentesis and chorionic villus sampling (CVS) tests.

An alternative to these invasive approaches has been developed for prenatal screening, e.g., to detecting fetal abnormalities, following the discovery that circulating cell-free fetal nucleic acid can be detected in maternal plasma and serum (Lo et al., Lancet 350:485-487, 1997; and U.S. Pat. No. 6,258,540). Circulating cell free fetal nucleic acid (cffNA) has several advantages making it more applicable for non-invasive prenatal testing. For example, cell free nucleic acid is present at higher levels than fetal cells and at concentrations sufficient for genetic analysis. Also, cffNA is cleared from the maternal bloodstream within hours after delivery, preventing contamination from previous pregnancies.

Examples of prenatal tests performed by detecting fetal DNA in maternal plasma or serum include fetal rhesus D (RhD) genotyping (Lo et al., N. Engl. J. Med. 339:1734-1738, 1998), fetal sex determination (Costa et al., N. Engl. J. Med. 346:1502, 2002), and diagnosis of several fetal disorders (Amicucci et al., Clin. Chem. 46:301-302, 2000; Saito et al., Lancet 356:1170, 2000; and Chiu et al., Lancet 360:998-1000, 2002). In addition, quantitative abnormalities of fetal DNA in maternal plasma/serum have been reported in preeclampsia (Lo et al., Clin. Chem. 45:184-188, 1999 and Zhong et al., Am. J. Obstet. Gynecol. 184:414-419, 2001), fetal trisomy 21 (Lo et al., Clin. Chem. 45:1747-1751, 1999 and Zhong et al., Prenat. Diagn. 20:795-798, 2000) and hyperemesis gravidarum (Sekizawa et al., Clin. Chem. 47:2164-2165, 2001).

### SUMMARY

The technology herein provides inter alia human epigenetic biomarkers that are useful for the noninvasive detection of fetal genetic traits, including, but not limited to, the presence or absence of fetal nucleic acid, the absolute or relative amount of fetal nucleic acid, fetal sex, and fetal chromosomal abnormalities such as aneuploidy. The human epigenetic biomarkers of the technology herein represent genomic DNA that display differential CpG methylation patterns between the fetus and mother. The compositions and processes of the technology herein allow for the detection and quantification of fetal nucleic acid in a maternal sample based on the methylation status of the nucleic acid in said sample. More specifically, the amount of fetal nucleic acid from a maternal sample can be determined relative to the total amount of nucleic acid present, thereby providing the percentage of fetal nucleic acid in the sample. Further, the amount of fetal nucleic acid can be determined in a sequence-specific (or locus-specific) manner and with sufficient sensitivity to allow for accurate chromosomal dosage analysis (for example, to detect the presence or absence of a fetal aneuploidy).

In the first aspect of the technology herein, a method is provided for enriching fetal nucleic acids from a maternal biological sample, based on differential methylation between fetal and maternal nucleic acid comprising the steps of: (a) binding a target nucleic acid, from a sample, and a control nucleic acid, from the sample, to a methylation-specific binding protein; and (b) eluting the bound nucleic acid based on methylation status, where differentially methylated nucleic acids elute at least partly into separate fractions. In an embodiment, the nucleic acid sequence includes one or more of the polynucleotide sequences of SEQ ID NOs: 1-261. SEQ ID NOs: 1-261 are provided in Tables 4A-4C. The technology herein includes the sequences of SEQ ID NOs: 1-261, and variations thereto. In an embodiment, a control nucleic acid is not included in step (a).

In a related embodiment, a method is provided for enriching fetal nucleic acid from a maternal sample, which comprises the following steps: (a) obtaining a biological sample from a woman; (b) separating fetal and maternal nucleic acid based on the methylation status of a CpG-containing genomic sequence in the sample, where the genomic sequence from the fetus and the genomic sequence from the woman are differentially methylated, thereby distinguishing the genomic sequence from the woman and the genomic sequence from the fetus in the sample. In an embodiment, the genomic sequence is at least 15 nucleotides in length, comprising at least one cytosine, further where the region consists of (1) a genomic locus selected from Tables 1A-1C; and (2) a DNA sequence of no more than 10 kb upstream and/or downstream from the locus. For this aspect and all aspects of the technology herein, obtaining a biological sample from a woman is not meant to limit the scope of the technology herein. Said obtaining can refer to actually drawing a sample from a woman (e.g., a blood draw) or to receiving a sample from elsewhere (e.g., from a clinic or hospital) and performing the remaining steps of the method.

In a related embodiment, a method is provided for enriching fetal nucleic acid from a maternal sample, which comprises the following steps: (a) obtaining a biological sample from the woman; (b) digesting or removing maternal nucleic acid based on the methylation status of a CpG-containing genomic sequence in the sample, where the genomic sequence from the fetus and the genomic sequence from the woman are differentially methylated, thereby enriching for the genomic sequence from the fetus in the sample. Maternal nucleic acid may be digested using one or more methylation sensitive restriction enzymes that selectively digest or cleave maternal nucleic acid based on its methylation status. In an embodiment, the genomic sequence is at least 15 nucleotides in length, comprising at least one cytosine, further where the region consists of (1) a genomic locus selected from Tables 1A-1C; and (2) a DNA sequence of no more than 10 kb upstream and/or downstream from the locus.

In a second aspect of the technology herein, a method is provided for preparing nucleic acid having a nucleotide sequence of a fetal nucleic acid, which comprises the following steps: (a) providing a sample from a pregnant female; (b) separating fetal nucleic acid from maternal nucleic acid from the sample of the pregnant female according to a different methylation state between the fetal nucleic acid and the maternal nucleic acid counterpart, where the nucleotide sequence of the fetal nucleic acid comprises one or more CpG sites from one or more of the polynucleotide sequences of SEQ ID NOs: 1-261 within a polynucleotide sequence from a gene or locus that contains one of the polynucleotide sequences of SEQ ID NOs: 1-261; and (c) preparing nucleic acid comprising a nucleotide sequence of the fetal nucleic acid by an amplification process in which fetal nucleic acid separated in part (b) is utilized as a template. In an embodiment, a method is provided for preparing nucleic acid having a nucleotide sequence of a fetal nucleic acid, which comprises the following steps: (a) providing a sample from a pregnant female; (b) digesting or removing maternal nucleic acid from the sample of the pregnant female according to a different methylation state between the fetal nucleic acid and the maternal nucleic acid counterpart, where the nucleotide sequence of the fetal nucleic acid comprises one or more CpG sites from one or more of the polynucleotide sequences of SEQ ID NOs: 1-261 within a polynucleotide sequence from a gene that contains one of the polynucleotide sequences of SEQ ID NOs: 1-261; and (c) preparing nucleic acid comprising a nucleotide sequence of the fetal nucleic acid. The preparing process of step (c) may be a hybridization process, a capture process, or an amplification process in which fetal nucleic acid separated in part (b) is utilized as a template. Also, in the above embodiment where maternal nucleic acid is digested, the maternal nucleic acid may be digested using one or more methylation sensitive restriction enzymes that selectively digest or cleave maternal nucleic acid based on its methylation status. In either embodiment, the polynucleotide sequences of SEQ ID NOs: 1-261 may be within a polynucleotide sequence from a CpG island that contains one of the polynucleotide sequences of SEQ ID NOs: 1-261. The polynucleotide sequences of SEQ ID NOs: 1-261 are further characterized in Tables 1-3 herein, including the identification of CpG islands that overlap with the polynucleotide sequences provided in SEQ ID NOs: 1-261. In an embodiment, the nucleic acid prepared by part (c) is in solution. In yet an embodiment, the method further comprises quantifying the fetal nucleic acid from the amplification process of step (c).

In a third aspect of the technology herein, a method is provided for enriching fetal nucleic acid from a sample from a pregnant female with respect to maternal nucleic acid, which comprises the following steps: (a) providing a sample from a pregnant female; and (b) separating or capturing fetal nucleic acid from maternal nucleic acid from the sample of the pregnant female according to a different methylation state between the fetal nucleic acid and the maternal nucleic acid, where the nucleotide sequence of the fetal nucleic acid comprises one or more CpG sites from one or more of the polynucleotide sequences of SEQ ID NOs: 1-261 within a polynucleotide sequence from a gene that contains one of the polynucleotide sequences of SEQ ID NOs: 1-261. In an embodiment, the polynucleotide sequences of SEQ ID NOs: 1-261 may be within a polynucleotide sequence from a CpG island that contains one of the polynucleotide sequences of SEQ ID NOs: 1-261. The polynucleotide sequences of SEQ ID NOs: 1-261 are characterized in Tables 1A-1C herein. In an embodiment, the nucleic acid separated by part (b) is in solution. In yet an embodiment, the method further comprises amplifying and/or quantifying the fetal nucleic acid from the separation process of step (b).

In a fourth aspect of the technology herein, a composition is provided comprising an isolated nucleic acid from a fetus of a pregnant female, where the nucleotide sequence of the nucleic acid comprises one or more of the polynucleotide sequences of SEQ ID NOs: 1-261. In one embodiment, the nucleotide sequence consists essentially of a nucleotide sequence of a gene, or portion thereof. In an embodiment, the nucleotide sequence consists essentially of a nucleotide sequence of a CpG island, or portion thereof. The polynucleotide sequences of SEQ ID NOs: 1-261 are further characterized in Tables 1A-1C. In an embodiment, the nucleic acid is in solution.

In an embodiment, the nucleic acid from the fetus is enriched relative to maternal nucleic acid. In an embodiment, the composition further comprises an agent that binds to methylated nucleotides.

For example, the agent may be a methyl-CpG binding protein (MBD) or fragment thereof.

In a fifth aspect of the technology herein, a composition is provided comprising an isolated nucleic acid from a fetus of a pregnant female, where the nucleotide sequence of the nucleic acid comprises one or more CpG sites from one or more of the polynucleotide sequences of SEQ ID NOs: 1-261 within a polynucleotide sequence from a gene, or portion thereof, that contains one of the polynucleotide sequences of SEQ ID NOs: 1-261. In an embodiment, the nucleotide sequence of the nucleic acid comprises one or more CpG sites from one or more of the polynucleotide sequences of SEQ ID NOs: 1-261 within a polynucleotide sequence from a CpG island, or portion thereof, that contains one of the polynucleotide sequences of SEQ ID NOs: 1-261. The polynucleotide sequences of SEQ ID NOs: 1-261 are further characterized in Tables 1A-1C. In an embodiment, the nucleic acid is in solution. In an embodiment, the nucleic acid from the fetus is enriched relative to maternal nucleic acid. Hyper- and hypomethylated nucleic acid sequences of the technology herein are identified in Tables 1A-1C. In an embodiment, the composition further comprises an agent that binds to methylated nucleotides. For example, the agent may be a methyl-CpG binding protein (MBD) or fragment thereof.

In some embodiments, a nucleotide sequence of the technology herein includes three or more of the CpG sites. In an embodiment, the nucleotide sequence includes five or more of the CpG sites. In an embodiment, the nucleotide sequence is from a gene region that comprises a PRC2 domain (see Table 3). In an embodiment, the nucleotide sequence is from a gene region involved with development. For example, SOX14—which is an epigenetic marker of the present technology (See Table 1A)—is a member of the SOX (SRY-related HMG-box) family of transcription factors involved in the regulation of embryonic development and in the determination of cell fate.

In some embodiments, the genomic sequence from the woman is methylated and the genomic sequence from the fetus is unmethylated. In other embodiments, the genomic sequence from the woman is unmethylated and the genomic sequence from the fetus is methylated. In an embodiment, the genomic sequence from the fetus is hypermethylated relative to the genomic sequence from the mother. Fetal genomic sequences found to be hypermethylated relative to maternal genomic sequence are provided in SEQ ID NOs: 1-59, 90-163, 176, 179, 180, 184, 188, 189, 190, 191, 193, 195, 198, 199, 200, 201, 202, 203, 205, 206, 207, 208, 209, 210, 211, 212, 213, 214, 221, 223, 225, 226, 231, 232, 233, 235, 239, 241, 257, 258, 259, and 261. Alternatively, the genomic sequence from the fetus is hypomethylated relative to the genomic sequence from the mother. Fetal genomic sequences found to be hypomethylated relative to maternal genomic sequence are provided in SEQ ID NOs: 60-85, 164, 165, 166, 167, 168, 169, 170, 171, 172, 173, 174, 175, 177, 178, 181, 182, 183, 185, 186, 187, 192, 194, 196, 197, 204, 215, 216, 217, 218, 219, 220, 222, 224, 227, 228, 229, 230, 234, 236, 237, 238, 240, 242, 243, 244, 245, 246, 247, 248, 249, 250, 251, 252, 253, 254, 255, 256, and 260. Methylation sensitive restriction enzymes of the technology herein may be sensitive to hypo- or hyper-methylated nucleic acid.

In an embodiment, the fetal nucleic acid is extracellular nucleic acid. Generally the extracellular fetal nucleic acid is about 500, 400, 300, 250, 200 or 150 (or any number there between) nucleotide bases or less. In an embodiment, the digested maternal nucleic acid is less than about 90, 100, 110, 120, 130, 140 or 150 base pairs. In a related embodiment, the fetal nucleic acid is selectively amplified, captured or separated from or relative to the digested maternal nucleic acid based on size. For example, PCR primers may be designed to amplify nucleic acid greater than about 75, 80, 85, 90, 95, 100, 105, 110, 115 or 120 (or any number there between) base pairs thereby amplifying fetal nucleic acid and not digested maternal nucleic acid. In an embodiment, the nucleic acid is subjected to fragmentation prior to the methods of the technology herein. Examples of methods of fragmenting nucleic acid, include but are not limited to sonication and restriction enzyme digestion. In some embodiments the fetal nucleic acid is derived from the placenta. In other embodiments the fetal nucleic acid is apoptotic.

In some embodiments, the present technology provides a method in which the sample is a member selected from the following: maternal whole blood, maternal plasma or serum, amniotic fluid, a chorionic villus sample, biopsy material from a pre-implantation embryo, fetal nucleated cells or fetal cellular remnants isolated from maternal blood, maternal urine, maternal saliva, washings of the female reproductive tract and a sample obtained by celocentesis or lung lavage. In certain embodiments, the biological sample is maternal blood. In some embodiments, the biological sample is a chorionic villus sample. In certain embodiments, the maternal sample is enriched for fetal nucleic acid prior to the methods of the present technology. Examples of fetal enrichment methods are provided in PCT Publication Nos. WO/2007140417A2, WO2009/032781A2 and US Publication No. 20050164241.

In some embodiments, all nucleated and anucleated cell populations are removed from the sample prior to practicing the methods of the technology herein. In some embodiments, the sample is collected, stored or transported in a manner known to the person of ordinary skill in the art to minimize degradation or the quality of fetal nucleic acid present in the sample.

The sample can be from any animal, including but not limited, human, non-human, mammal, reptile, cattle, cat, dog, goat, swine, pig, monkey, ape, gorilla, bull, cow, bear, horse, sheep, poultry, mouse, rat, fish, dolphin, whale, and shark, or any animal or organism that may have a detectable pregnancy-associated disorder or chromosomal abnormality.

In some embodiments, the sample is treated with a reagent that differentially modifies methylated and unmethylated DNA. For example, the reagent may comprise bisulfite; or the reagent may comprise one or more enzymes that preferentially cleave methylated DNA; or the reagent may comprise one or more enzymes that preferentially cleave unmethylated DNA. Examples of methylation sensitive restriction enzymes include, but are not limited to, HhaI and HpaII.

In one embodiment, the fetal nucleic acid is separated from the maternal nucleic acid by an agent that specifically binds to methylated nucleotides in the fetal nucleic acid. In an embodiment, the fetal nucleic acid is separated or removed from the maternal nucleic acid by an agent that specifically binds to methylated nucleotides in the maternal nucleic acid counterpart. In an embodiment, the agent that binds to methylated nucleotides is a methyl-CpG binding protein (MBD) or fragment thereof.

In a sixth aspect of the technology herein, a method is provided for determining the amount or copy number of fetal DNA in a maternal sample that comprises differentially methylated maternal and fetal DNA. The method is performed by a) distinguishing between the maternal and fetal DNA based on differential methylation status; and b) quantifying the fetal DNA of step a). In a specific embodiment, the method comprises a) digesting the maternal DNA in a maternal sample using one or more methylation sensitive restriction enzymes thereby enriching the fetal DNA; and b) determining the amount of fetal DNA from step a). The amount of fetal DNA can be used inter alia to confirm the presence or absence of fetal nucleic acid, determine fetal sex, diagnose fetal disease or a pregnancy-associated disorder, or be used in conjunction with other fetal diagnostic methods to improve sensitivity or specificity. In one embodiment, the method for determining the amount of fetal DNA does not require the use of a polymorphic sequence. For example, an allelic ratio is not used to quantify the fetal DNA in step b). In an embodiment, the method for determining the amount of fetal DNA does not require the treatment of DNA with bisulfite to convert cytosine residues to uracil. Bisulfite is known to degrade DNA, thereby, further reducing the already limited fetal nucleic acid present in maternal samples. In one embodiment, determining the amount of fetal DNA in step b) is done by introducing one or more competitors at known concentrations. In an embodiment, determining the amount of fetal DNA in step b) is done by RT-PCR, primer extension, sequencing or counting. In a related embodiment, the amount of nucleic acid is determined using BEAMing technology as described in US Patent Publication No. US20070065823. In another related embodiment, the amount of nucleic acid is determined using the shotgun sequencing technology described in US Patent Publication No. US20090029377 (U.S. application Ser. No. 12/178,181), or variations thereof. In an embodiment, the restriction efficiency is determined and the efficiency rate is used to further determine the amount of fetal DNA. Exemplary differentially methylated nucleic acids are provided in SEQ ID NOs: 1-261.

In a seventh aspect of the technology herein, a method is provided for determining the concentration of fetal DNA in a maternal sample, where the maternal sample comprises differentially methylated maternal and fetal DNA, comprising a) determining the total amount of DNA present in the maternal sample; b) selectively digesting the maternal DNA in a maternal sample using one or more methylation sensitive restriction enzymes thereby enriching the fetal DNA; c) determining the amount of fetal DNA from step b); and d) comparing the amount of fetal DNA from step c) to the total amount of DNA from step a), thereby determining the concentration of fetal DNA in the maternal sample. The concentration of fetal DNA can be used inter alia in conjunction with other fetal diagnostic methods to improve sensitivity or specificity. In one embodiment, the method for determining the amount of fetal DNA does not require the use of a polymorphic sequence. For example, an allelic ratio is not used to quantify the fetal DNA in step b). In an embodiment, the method for determining the amount of fetal DNA does not require the treatment of DNA with bisulfite to convert cytosine residues to uracil. In one embodiment, determining the amount of fetal DNA in step b) is done by introducing one or more competitors at known concentrations. In an embodiment, determining the amount of fetal DNA in step b) is done by RT-PCR, sequencing or counting. In an embodiment, the restriction efficiency is determined and used to further determine the amount of total DNA and fetal DNA. Exemplary differentially methylated nucleic acids are provided in SEQ ID NOs: 1-261.

In an eighth aspect of the technology herein, a method is provided for determining the presence or absence of a fetal aneuploidy using fetal DNA from a maternal sample, where the maternal sample comprises differentially methylated maternal and fetal DNA, comprising a) selectively digesting the maternal DNA in a maternal sample using one or more methylation sensitive restriction enzymes thereby enriching the fetal DNA; b) determining the amount of fetal DNA from a target chromosome; c) determining the amount of fetal DNA from a reference chromosome; and d) comparing the amount of fetal DNA from step b) to step c), where a biologically or statistically significant difference between the amount of target and reference fetal DNA is indicative of the presence of a fetal aneuploidy. In one embodiment, the method for determining the amount of fetal DNA does not require the use of a polymorphic sequence. For example, an allelic ratio is not used to quantify the fetal DNA in step b). In an embodiment, the method for determining the amount of fetal DNA does not require the treatment of DNA with bisulfite to convert cytosine residues to uracil. In one embodiment, determining the amount of fetal DNA in steps b) and c) is done by introducing one or more competitors at known concentrations. In an embodiment, determining the amount of fetal DNA in steps b) and c) is done by RT-PCR, sequencing or counting. In an embodiment, the amount of fetal DNA from a target chromosome determined in step b) is compared to a standard control, for example, the amount of fetal DNA from a target chromosome from euploid pregnancies. In an embodiment, the restriction efficiency is determined and used to further determine the amount of fetal DNA from a target chromosome and from a reference chromosome. Exemplary differentially methylated nucleic acids are provided in SEQ ID NOs: 1-261.

In a ninth aspect of the technology herein, a method is provided for detecting the presence or absence of a chromosomal abnormality by analyzing the amount or copy number of target nucleic acid and control nucleic acid from a sample of differentially methylated nucleic acids comprising the steps of: (a) enriching a target nucleic acid, from a sample, and a control nucleic acid, from the sample, based on its methylation state; (b) performing a copy number analysis of the enriched target nucleic acid in at least one of the fractions; (c) performing a copy number analysis of the enriched control nucleic acid in at least one of the fractions; (d) comparing the copy number from step (b) with the copy number from step (c); and (e) determining if a chromosomal abnormality exists based on the comparison in step (d), where the target nucleic acid and control nucleic acid have the same or substantially the same methylation status. In a related embodiment, a method is provided for detecting the presence or absence of a chromosomal abnormality by analyzing the amount or copy number of target nucleic acid and control nucleic acid from a sample of differentially methylated nucleic acids comprising the steps of: (a) binding a target nucleic acid, from a sample, and a control nucleic acid, from the sample, to a binding agent; (b) eluting the bound nucleic acid based on methylation status, where differentially methylated nucleic acids elute at least partly into separate fractions; (c) performing a copy number analysis of the eluted target nucleic acid in at least one of the fractions; (d) performing a copy number analysis of the eluted control nucleic acid in at least one of the fractions; (e) comparing the copy number from step (c) with the copy number from step (d); and (f) determining if a chromosomal abnormality exists based on the comparison in step (e), where the target nucleic acid and control nucleic acid have the same or substantially the same methylation status. Differentially methylated nucleic acids are provided in SEQ ID NOs: 1-261.

In a tenth aspect of the technology herein, a method is provided for detecting the presence or absence of a chromosomal abnormality by analyzing the allelic ratio of target nucleic acid and control nucleic acid from a sample of differentially methylated nucleic acids comprising the steps of: (a) binding a target nucleic acid, from a sample, and a control nucleic acid, from the sample, to a binding agent; (b) eluting the bound nucleic acid based on methylation status, where differentially methylated nucleic acids elute at least partly into separate fractions; (c) performing an allelic ratio analysis of the eluted target nucleic acid in at least one of the fractions; (d) performing an allelic ratio analysis of the eluted control nucleic acid in at least one of the fractions; (e) comparing the allelic ratio from step c with the all from step d; and (f) determining if a chromosomal abnormality exists based on the comparison in step (e), where the target nucleic acid and control nucleic acid have the same or substantially the same methylation status. Differentially methylated nucleic acids are provided in SEQ ID NOs: 1-261, and SNPs within the differentially methylated nucleic acids are provided in Table 2. The methods may also be useful for detecting a pregnancy-associated disorder.

In an eleventh aspect of the technology herein, the amount of maternal nucleic acid is determined using the methylation-based methods of the technology herein. For example, fetal nucleic acid can be separated (for example, digested using a methylation-sensitive enzyme) from the maternal nucleic acid in a sample, and the maternal nucleic acid can be quantified using the methods of the technology herein. Once the amount of maternal nucleic acid is determined, that amount can subtracted from the total amount of nucleic acid in a sample to determine the amount of fetal nucleic acid. The amount of fetal nucleic acid can be used to detect fetal traits, including fetal aneuploidy, as described herein.

For all aspects and embodiments of the technology described herein, the methods may also be useful for detecting a pregnancy-associated disorder. In some embodiments, the sample comprises fetal nucleic acid, or fetal nucleic acid and maternal nucleic acid. In the case when the sample comprises fetal and maternal nucleic acid, the fetal nucleic acid and the maternal nucleic acid may have a different methylation status. Nucleic acid species with a different methylation status can be differentiated by any method known in the art. In an embodiment, the fetal nucleic acid is enriched by the selective digestion of maternal nucleic acid by a methylation sensitive restriction enzyme. In an embodiment, the fetal nucleic acid is enriched by the selective digestion of maternal nucleic acid using two or more methylation sensitive restriction enzymes in the same assay. In an embodiment, the target nucleic acid and control nucleic acid are both from the fetus.

In an embodiment, the average size of the fetal nucleic acid is about 100 bases to about 500 bases in length. In an embodiment the chromosomal abnormality is an aneuploidy, such as trisomy 21.

In some embodiments, the target nucleic acid is at least a portion of a chromosome which may be abnormal and the control nucleic acid is at least a portion of a chromosome which is very rarely abnormal. For example, when the target nucleic acid is from chromosome 21, the control nucleic acid is from a chromosome other than chromosome 21—preferably another autosome. In an embodiment, the binding agent is a methylation-specific binding protein such as MBD-Fc. Also, the enriched or eluted nucleic acid is amplified and/or quantified by any method known in the art. In an embodiment, the fetal DNA is quantified using a method that does not require the use of a polymorphic sequence. For example, an allelic ratio is not used to quantify the fetal DNA. In an embodiment, the method for quantifying the amount of fetal DNA does not require the treatment of DNA with bisulfite to convert cytosine residues to uracil.

In some embodiments, the methods of the technology herein include the additional step of determining the amount of one or more Y-chromosome-specific sequences in a sample. In a related embodiment, the amount of fetal nucleic acid in a sample as determined by using the methylation-based methods of the technology herein is compared to the amount of Y-chromosome nucleic acid present.

Methods for differentiating nucleic acid based on methylation status include, but are not limited to, methylation sensitive capture, for example using, MBD2-Fc fragment; bisulfite conversion methods, for example, MSP (methylation-sensitive PCR), COBRA, methylation-sensitive single nucleotide primer extension (Ms-SNuPE) or Sequenom MassCLEAVE™ technology; and the use of methylation sensitive restriction enzymes. Except where explicitly stated, any method for differentiating nucleic acid based on methylation status can be used with the compositions and methods of the technology herein.

In some embodiments, methods of the technology herein may further comprise an amplification step. The amplification step can be performed by PCR, such as methylation-specific PCR. In an embodiment, the amplification reaction is performed on single molecules, for example, by digital PCR, which is further described in U.S. Pat. Nos. 6,143,496 and 6,440,706, both of which are hereby incorporated by reference. In other embodiments, the method does not require amplification. For example, the amount of enriched fetal DNA may be determined by counting the fetal DNA (or sequence tags attached thereto) with a flow cytometer or by sequencing means that do not require amplification. In an embodiment, the amount of fetal DNA is determined by an amplification reaction that generates amplicons larger than the digested maternal nucleic acid, thereby further enriching the fetal nucleic acid.

In some embodiments, the fetal nucleic acid (alone or in combination with the maternal nucleic acid) comprises one or more detection moieties. In one embodiment, the detection moiety may be any one or more of a compomer, sugar, peptide, protein, antibody, chemical compound (e.g., biotin), mass tag (e.g., metal ions or chemical groups), fluorescent tag, charge tag (e.g., such as polyamines or charged dyes) and hydrophobic tag. In a related embodiment, the detection moiety is a mass-distinguishable product (MDP) or part of an MDP detected by mass spectrometry. In a specific embodiment, the detection moiety is a fluorescent tag or label that is detected by mass spectrometry. In some embodiments, the detection moiety is at the 5′ end of a detector oligonucleotide, the detection moiety is attached to a non-complementary region of a detector oligonucleotide, or the detection moiety is at the 5′ terminus of a non-complementary sequence. In certain embodiments, the detection moiety is incorporated into or linked to an internal nucleotide or to a nucleotide at the 3′ end of a detector oligonucleotide. In some embodiments, one or more detection moieties are used either alone or in combination. See for example US Patent Applications US20080305479 and US20090111712. In certain embodiments, a detection moiety is cleaved by a restriction endonuclease, for example, as described in U.S. application Ser. No. 12/726,246. In some embodiments, a specific target chromosome is labeled with a specific detection moiety and one or more non-target chromosomes are labeled with a different detection moiety, whereby the amount target chromosome can be compared to the amount of non-target chromosome.

For embodiments that require sequence analysis, any one of the following sequencing technologies may be used: a primer extension method (e.g., iPLEX®; Sequenom, Inc.), direct DNA sequencing, restriction fragment length polymorphism (RFLP analysis), real-time PCR, for example using “STAR” (Scalable Transcription Analysis Routine) technology (see U.S. Pat. No. 7,081,339), or variations thereof, allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, fluorescence tagged dNTP/ddNTPs, AlphaScreen, SNPstream, genetic bit analysis (GBA), Multiplex minisequencing, SNaPshot, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Invader™ assay, hybridization using at least one probe, hybridization using at least one fluorescently labeled probe, electrophoresis, cloning and sequencing, for example as performed on the 454 platform (Roche) (Margulies, M. et al. 2005 Nature 437, 376-380), Illumina Genome Analyzer (or Solexa platform) or SOLiD System (Applied Biosystems) or the Helicos True Single Molecule DNA sequencing technology (Harris T D et al. 2008 Science, 320, 106-109), the single molecule, real-time (SMRT™) technology of Pacific Biosciences, or nanopore-based sequencing (Soni G V and Meller A. 2007 Clin Chem 53: 1996-2001), for example, using an Ion Torrent ion sensor that measures an electrical charge associated with each individual base of DNA as each base passes through a tiny pore at the bottom of a sample well, or Oxford Nanopore device that uses a nanopore to measure the electrical charge associated with each individual unit of DNA, and combinations thereof. Nanopore-based methods may include sequencing nucleic acid using a nanopore, or counting nucleic acid molecules using a nanopore, for example, based on size where sequence information is not determined.

The absolute copy number of one or more nucleic acids can be determined, for example, using mass spectrometry, a system that uses a competitive PCR approach for absolute copy number measurements. See for example, Ding C, Cantor C R (2003) A high-throughput gene expression analysis technique using competitive PCR and matrix-assisted laser desorption ionization time-of-flight MS. Proc Natl Acad Sci USA 100:3059-3064, and U.S. patent application Ser. No. 10/655,762, which published as US Patent Publication No. 20040081993, both of which are hereby incorporated by reference.

In some embodiments, the amount of the genomic sequence is compared with a standard control, where an increase or decrease from the standard control indicates the presence or progression of a pregnancy-associated disorder. For example, the amount of fetal nucleic acid may be compared to the total amount of DNA present in the sample. Or when detecting the presence or absence of fetal aneuploidy, the amount of fetal nucleic acid from target chromosome may be compared to the amount of fetal nucleic acid from a reference chromosome. Preferably the reference chromosome is another autosome that has a low rate of aneuploidy. The ratio of target fetal nucleic acid to reference fetal nucleic acid may be compared to the same ratio from a normal, euploid pregnancy. For example, a control ratio may be determined from a DNA sample obtained from a female carrying a healthy fetus who does not have a chromosomal abnormality. Preferably, one uses a panel of control samples. Where certain chromosome anomalies are known, one can also have standards that are indicative of a specific disease or condition. Thus, for example, to screen for three different chromosomal aneuploidies in a maternal plasma of a pregnant female, one preferably uses a panel of control DNAs that have been isolated from mothers who are known to carry a fetus with, for example, chromosome 13, 18, or 21 trisomy, and a mother who is pregnant with a fetus who does not have a chromosomal abnormality.

In some embodiments, the present technology provides a method in which the alleles from the target nucleic acid and control nucleic acid are differentiated by sequence variation. The sequence variation may be a single nucleotide polymorphism (SNP) or an insertion/deletion polymorphism. In some embodiments, the fetal nucleic acid should comprise at least one high frequency heterozygous polymorphism (e.g., about 2%, 3%, 4%, 5%, 6%, 7%, 8%, 9%, 10%, 11%, 12%, 13%, 14%, 15%, 16%, 17%, 18%, 19%, 20%, 25%, 35%, 40%, 45%, 50%, 55%, 60% or more frequency rate), which allows the determination of the allelic-ratio of the nucleic acid in order to assess the presence or absence of the chromosomal abnormality. Lists of example SNPs are provided in Table 2, Table 9 and Table 10, however, these do not represent a complete list of polymorphic alleles that can be used as part of the technology herein. In some embodiments, any SNP meeting the following criteria, for example, may also be considered: (a) the SNP has a heterozygosity frequency greater than about 2% (preferably across a range of different populations), (b) the SNP is a heterozygous locus; and (c)(i) the SNP is within a nucleic acid sequence described herein, or (c)(iii) the SNP is within about 5 to about 2000 base pairs of a SNP described herein (e.g., within about 5, 10, 15, 20, 25, 30, 40, 50, 60, 70, 80, 90, 100, 200, 300, 400, 500, 600, 700, 800, 900, 1000, 1250, 1500, 1750 or 2000 base pairs of a SNP described herein). In some cases, SNPs are selected by other criteria described in further detail herein.

In other embodiments, the sequence variation is a short tandem repeat (STR) polymorphism. In some embodiments, the sequence variation falls in a restriction site, whereby one allele is susceptible to digestion by a restriction enzyme and the one or more other alleles are not. In some embodiments, the sequence variation is a methylation site.

In some embodiments, performing an allelic ratio analysis comprises determining the ratio of alleles of the target nucleic acid and control nucleic acid from the fetus of a pregnant woman by obtaining an nucleic acid-containing biological sample from the pregnant woman, where the biological sample contains fetal nucleic acid, partially or wholly separating the fetal nucleic acid from the maternal nucleic acid based on differential methylation, discriminating the alleles from the target nucleic acid and the control nucleic acid, followed by determination of the ratio of the alleles, and detecting the presence or absence of a chromosomal disorder in the fetus based on the ratio of alleles, where a ratio above or below a normal, euploid ratio is indicative of a chromosomal disorder. In one embodiment, the target nucleic acid is from a suspected aneuploid chromosome (e.g., chromosome 21) and the control nucleic acid is from a euploid chromosome from the same fetus.

In some embodiments, the present technology is combined with other fetal markers to detect the presence or absence of multiple chromosomal abnormalities, where the chromosomal abnormalities are selected from the following: trisomy 21, trisomy 18 and trisomy 13, or combinations thereof. In some embodiments, the chromosomal disorder involves the X chromosome or the Y chromosome.

In some embodiments, the compositions or processes may be multiplexed in a single reaction. For example, the amount of fetal nucleic acid may be determined at multiple loci across the genome. Or when detecting the presence or absence of fetal aneuploidy, the amount of fetal nucleic acid may be determined at multiple loci on one or more target chromosomes (e.g., chromosomes 13, 18 or 21) and on one or more reference chromosomes. If an allelic ratio is being used, one or more alleles from Table 2, Table 9, and/or Table 10 can be detected and discriminated simultaneously. When determining allelic ratios, multiplexing embodiments are particularly important when the genotype at a polymorphic locus is not known. In some instances, for example when the mother and child are homozygous at the polymorphic locus, the assay may not be informative. In one embodiment, greater than 1, 2, 3, 4, 5, 6, 7, 8, 9, 10, 11, 12, 13, 14, 15, 16, 17, 18, 19, 20, 25, 30, 35, 40, 50, 100, 200, 300 or 500, and any intermediate levels, polynucleotide sequences of the technology herein are enriched, separated and/or examined according the methods of the technology. When detecting a chromosomal abnormality by analyzing the copy number of target nucleic acid and control nucleic acid, less than 3, 4, 5, 6, 7, 8, 9, 10, 11, 12, 13, or 14 polynucleotide sequences may need to be analyzed to accurately detect the presence or absence of a chromosomal abnormality. In an embodiment, the compositions or processes of the technology herein may be used to assay samples that have been divided into 2, 3, 4, 5, 6, 7, 8, 9, 10, 11, 12, 13, 14, 15, 16, 17, 18, 19, 20, 25, 30, 35, 40, 50, 100 or more replicates, or into single molecule equivalents. Methods for analyzing fetal nucleic acids from a maternal sample in replicates, including single molecule analyses, are provided in U.S. application Ser. No. 11/364,294, which published as US Patent Publication No. US 2007-0207466 A1, which is hereby incorporated by reference.

In a further embodiment, the present technology provides a method where a comparison step shows an increased risk of a fetus having a chromosomal disorder if the ratio of the alleles or absolute copy number of the target nucleic acid is higher or lower by 1 standard deviation from the standard control sequence. In some embodiments, the comparison step shows an increased risk of a fetus having a chromosomal disorder if the ratio of the alleles or absolute copy number of the target nucleic acid is higher or lower by 2 standard deviations from the standard control sequence. In some other embodiments, the comparison step shows an increased risk of a fetus having a chromosomal disorder if the ratio of the alleles or absolute copy number of the target nucleic acid is higher or lower by 3 standard deviations from the standard control sequence. In some embodiments, the comparison step shows an increased risk of a fetus having a chromosomal disorder if the ratio of the alleles or absolute copy number of the target nucleic acid is higher or lower than a statistically significant standard deviation from the control. In one embodiment, the standard control is a maternal reference, and in an embodiment the standard control is a fetal reference chromosome (e.g., non-trisomic autosome).

In some embodiments, the methods of the technology herein may be combined with other methods for diagnosing a chromosomal abnormality. For example, a noninvasive diagnostic method may require confirmation of the presence or absence of fetal nucleic acid, such as a sex test for a female fetus or to confirm an RhD negative female fetus in an RhD negative mother. In an embodiment, the compositions and methods of the technology herein may be used to determine the percentage of fetal nucleic acid in a maternal sample in order to enable another diagnostic method that requires the percentage of fetal nucleic acid be known. For example, does a sample meet certain threshold concentration requirements? When determining an allelic ratio to diagnose a fetal aneuploidy from a maternal sample, the amount or concentration of fetal nucleic acid may be required to make a diagnose with a given sensitivity and specificity. In other embodiments, the compositions and methods of the technology herein for detecting a chromosomal abnormality can be combined with other known methods thereby improving the overall sensitivity and specificity of the detection method. For example, mathematical models have suggested that a combined first-trimester screening program utilizing maternal age (MA), nuchal translucency (NT) thickness, serum-free beta-hCG, and serum PAPP-A will detect more than 80% of fetuses with Down's syndrome for a 5% invasive testing rate (Wald and Hackshaw, Prenat Diagn 17(9):921-9 (1997)). However, the combination of commonly used aneuploidy detection methods combined with the non-invasive free fetal nucleic acid-based methods described herein may offer improved accuracy with a lower false positive rate. Examples of combined diagnostic methods are provided in PCT Publication Number WO2008157264A2 (assigned to the Applicant), which is hereby incorporated by reference. In some embodiments, the methods of the technology herein may be combined with cell-based methods, where fetal cells are procured invasively or non-invasively.

In certain embodiments, an increased risk for a chromosomal abnormality is based on the outcome or result(s) produced from the compositions or methods provided herein. An example of an outcome is a deviation from the euploid absolute copy number or allelic ratio, which indicates the presence of chromosomal aneuploidy. This increase or decrease in the absolute copy number or ratio from the standard control indicates an increased risk of having a fetus with a chromosomal abnormality (e.g., trisomy 21). Information pertaining to a method described herein, such as an outcome, result, or risk of trisomy or aneuploidy, for example, may be transfixed, renditioned, recorded and/or displayed in any suitable medium. For example, an outcome may be transfixed in a medium to save, store, share, communicate or otherwise analyze the outcome. A medium can be tangible (e.g., paper) or intangible (e.g., electronic medium), and examples of media include, but are not limited to, computer media, databases, charts, patient charts, records, patient records, graphs and tables, and any other medium of expression. The information sometimes is stored and/or renditioned in computer readable form and sometimes is stored and organized in a database. In certain embodiments, the information may be transferred from one location to another using a physical medium (e.g., paper) or a computer readable medium (e.g., optical and/or magnetic storage or transmission medium, floppy disk, hard disk, random access memory, computer processing unit, facsimile signal, satellite signal, transmission over an internet or transmission over the world-wide web).

In practicing the present technology within all aspects mentioned above, a CpG island may be used as the CpG-containing genomic sequence in some cases, whereas in other cases the CpG-containing genomic sequence may not be a CpG island.

In some embodiments, the present technology provides a kit for performing the methods of the technology. One component of the kit is a methylation-sensitive binding agent.

Also provided, in some aspects, are methods for determining the amount of fetal nucleic acid in a sample comprising (a) contacting a sample nucleic acid with one or more agents that differentially modify methylated nucleic acid and unmethylated nucleic acid, which sample nucleic acid comprises differentially methylated fetal nucleic acid and maternal nucleic acid, the combination of the fetal nucleic acid and the maternal nucleic acid comprising total nucleic acid in the sample, thereby generating differentially modified sample nucleic acid; (b) contacting under amplification conditions the differentially modified sample nucleic acid with: (i) a first set of amplification primers that specifically amplify a first region in sample nucleic acid comprising one or more loci that are differentially methylated between the fetal nucleic acid and maternal nucleic acid, and (ii) a second set of amplification primers that amplify a second region in the sample nucleic acid allowing for a determination of total nucleic acid in the sample, where the first region and the second region are different, thereby generating fetal nucleic acid amplification products and total nucleic acid amplification products; (c) incorporating adaptor oligonucleotides into the amplification products in (b); thereby generating adaptor-modified amplification products; (d) obtaining nucleotide sequences of the adaptor-modified amplification products in (c) by a sequencing process, thereby generating sequence reads; (e) quantifying the sequence reads; and (f) determining the amount of fetal nucleic acid in the sample based on a quantification of the sequence reads in (e).

Also provided, in some aspects, are methods for determining the amount of fetal nucleic acid in a sample comprising (a) contacting a sample nucleic acid with one or more methylation sensitive restriction enzymes, which sample nucleic acid comprises differentially methylated fetal nucleic acid and maternal nucleic acid, the combination of the fetal nucleic acid and the maternal nucleic acid comprising total nucleic acid in the sample, thereby generating differentially digested sample nucleic acid; (b) contacting under amplification conditions the digested sample nucleic acid with (i) a first set of amplification primers that specifically amplify a first region in sample nucleic acid comprising one or more loci that are differentially methylated between the fetal nucleic acid and maternal nucleic acid, and (ii) a second set of amplification primers that amplify a second region in the sample nucleic acid allowing for a determination of total nucleic acid in the sample, where the first region and the second region are different, thereby generating fetal nucleic acid amplification products and total nucleic acid amplification products; (c) incorporating adaptor oligonucleotides into the amplification products in (b); thereby generating adaptor-modified amplification products; (d) obtaining nucleotide sequences of the adaptor-modified amplification products in (c) by a sequencing process, thereby generating sequence reads; (e) quantifying the sequence reads; and (f) determining the amount of fetal nucleic acid in the sample based on a quantification of the sequence reads in (e).

Also provided, in some aspects, are methods for determining the copy number of fetal nucleic acid in a sample comprising (a) contacting a sample nucleic acid with one or more agents that differentially modify methylated nucleic acid and unmethylated nucleic acid, which sample nucleic acid comprises differentially methylated fetal nucleic acid and maternal nucleic acid, the combination of the fetal nucleic acid and the maternal nucleic acid comprising total nucleic acid in the sample, thereby generating differentially modified sample nucleic acid; (b) contacting under amplification conditions the differentially modified sample nucleic acid with (i) a first set of amplification primers that specifically amplify a first region in sample nucleic acid comprising one or more loci that are differentially methylated between the fetal nucleic acid and maternal nucleic acid, and (ii) a predetermined copy number of one or more first competitor oligonucleotides that compete with the first region for hybridization of primers of the first amplification primer set, thereby generating fetal nucleic acid amplification products and competitor amplification products; (c) incorporating adaptor oligonucleotides into the amplification products in (b); thereby generating adaptor-modified amplification products; (d) obtaining nucleotide sequences of the adaptor-modified amplification products in (c) by a sequencing process, thereby generating sequence reads; (e) quantifying the sequence reads; and (f) determining the copy number of fetal nucleic acid in the sample based on a quantification of the sequence reads in (e) and the amount of competitor oligonucleotide used.

Also provided, in some aspects, are methods for detecting the presence or absence of a fetal aneuploidy in a sample comprising (a) contacting a sample nucleic acid with one or more agents that differentially modify methylated nucleic acid and unmethylated nucleic acid, which sample nucleic acid comprises differentially methylated fetal nucleic acid and maternal nucleic acid, the combination of the fetal nucleic acid and the maternal nucleic acid comprising total nucleic acid in the sample, thereby generating differentially modified sample nucleic acid; (b) contacting under amplification conditions the differentially modified sample nucleic acid with (i) a first set of amplification primers that specifically amplify one or more loci in a target chromosome that are differentially methylated between the fetal nucleic acid and maternal nucleic acid, and (ii) a second set of amplification primers that specifically amplify one or more loci in a reference chromosome that are differentially methylated between the fetal nucleic acid and maternal nucleic acid, thereby generating target chromosome amplification products and reference chromosome amplification products; (c) incorporating adaptor oligonucleotides into the amplification products in (b); thereby generating adaptor-modified amplification products; (d) obtaining nucleotide sequences of the adaptor-modified amplification products in (c) by a sequencing process, thereby generating sequence reads; (e) quantifying the sequence reads; and (f) detecting the presence or absence of a fetal aneuploidy in the sample based on a quantification of the sequence reads in (e).

In some embodiments, the first region comprises one or more loci which each contain a restriction site for a methylation-sensitive restriction enzyme. In some embodiments, the one or more agents that differentially modify methylated nucleic acid and unmethylated nucleic acid comprise one or more methylation sensitive restriction enzymes. In some embodiments, the second region comprises one or more loci which do not contain a restriction site for a methylation-sensitive restriction enzyme. In some embodiments, the one or more agents that differentially modify methylated nucleic acid and unmethylated nucleic acid comprise bisulfite. In some embodiments, the target chromosome comprises one or more loci which each contain a restriction site for a methylation-sensitive restriction enzyme. In some embodiments, the reference chromosome comprises one or more loci which each contain a restriction site for a methylation-sensitive restriction enzyme.

In some embodiments, the adaptor oligonucleotides are incorporated into the amplification products by ligation. In some cases, the ligation is unidirectional ligation. In some embodiments, the adaptor oligonucleotides are incorporated into the amplification products using amplification primers comprising the adaptor oligonucleotide sequences. In some embodiments, the adaptor oligonucleotides comprise one or more index sequences. In some cases, the one or more index sequences comprise a sample-specific index. In some cases, the one or more index sequences comprise an aliquot-specific index.

In some embodiments, at least one of the one or more loci in the first region comprises a nucleotide sequence selected from among SEQ ID NOs:1-261, or a fragment thereof. In some cases, at least one of the one or more loci in the first region comprises a nucleotide sequence selected from among SEQ ID NOs:1-89, or a fragment thereof. In some cases, at least one of the one or more loci in the first region comprises a nucleotide sequence selected from among SEQ ID NOs:90-261, or a fragment thereof. In some cases, at least one of the one or more loci in the first region comprises a nucleotide sequence selected from among SEQ ID NOs:1-59 and SEQ ID NOs:86-89, or a fragment thereof. In some cases, at least one of the one or more loci in the first region comprises a nucleotide sequence selected from among SEQ ID NOs:1-59, or a fragment thereof. In some cases, at least one of the one or more loci in the first region comprises a nucleotide sequence selected from among SEQ ID NO:42, SEQ ID NO:52, SEQ ID NO:154, SEQ ID NO:158 and SEQ ID NO:163.

In some embodiments, at least one of the one or more loci in the target chromosome comprises a nucleotide sequence selected from among SEQ ID NOs:1-261, or a fragment thereof. In some cases, at least one of the one or more loci in the target chromosome comprises a nucleotide sequence selected from among SEQ ID NOs:1-89, or a fragment thereof. In some cases, at least one of the one or more loci in the target chromosome comprises a nucleotide sequence selected from among SEQ ID NOs:90-261, or a fragment thereof. In some cases, at least one of the one or more loci in target chromosome comprises a nucleotide sequence selected from among SEQ ID NOs:1-59 and SEQ ID NOs:86-89, or a fragment thereof. In some cases, at least one of the one or more loci in the target chromosome comprises a nucleotide sequence selected from among SEQ ID NOs:1-59, or a fragment thereof. In some cases, at least one of the one or more loci in the target chromosome comprises a nucleotide sequence selected from among SEQ ID NO:42, SEQ ID NO:52, SEQ ID NO:154, SEQ ID NO:158 and SEQ ID NO:163.

In some embodiments, at least one of the one or more loci in the reference chromosome comprises a nucleotide sequence selected from among SEQ ID NOs:1-261, or a fragment thereof. In some cases, at least one of the one or more loci in the reference chromosome comprises a nucleotide sequence selected from among SEQ ID NOs:1-89, or a fragment thereof. In some cases, at least one of the one or more loci in the reference chromosome comprises a nucleotide sequence selected from among SEQ ID NOs:90-261, or a fragment thereof. In some cases, at least one of the one or more loci in reference chromosome comprises a nucleotide sequence selected from among SEQ ID NOs:1-59 and SEQ ID NOs:86-89, or a fragment thereof. In some cases, at least one of the one or more loci in the reference chromosome comprises a nucleotide sequence selected from among SEQ ID NOs:1-59, or a fragment thereof. In some cases, at least one of the one or more loci in the reference chromosome comprises a nucleotide sequence selected from among SEQ ID NO:42, SEQ ID NO:52, SEQ ID NO:154, SEQ ID NO:158 and SEQ ID NO:163.

In some embodiments, the sequencing process is a sequencing by synthesis method. In some embodiments, the sequencing process is a reversible terminator-based sequencing method.

In some embodiments, the amount of fetal nucleic acid determined is the fraction of fetal nucleic acid in the sample based on the amount of each of the fetal nucleic acid amplification products and total nucleic acid amplification products. In some cases, the fraction of fetal nucleic acid is a ratio of fetal nucleic acid amplification product amount to total nucleic acid amplification product amount.

In some embodiments, a method further comprises contacting under amplification conditions the nucleic acid sample with a second set of amplification primers that amplify a second region in the sample nucleic acid allowing for a determination of total nucleic acid in the sample, where the first region and the second region are different. In some cases, the second region comprises one or more loci which do not contain a restriction site for a methylation-sensitive restriction enzyme.

In some embodiments, a method further comprises contacting under amplification conditions the nucleic acid sample with a third set of amplification primers that amplify a third region in the sample nucleic acid allowing for a determination of the presence or absence of fetal specific nucleic acid. In some cases, the fetal specific nucleic acid is Y chromosome nucleic acid. In some cases, the third region comprises one or more loci within chromosome Y.

In some embodiments, a method further comprises contacting under amplification conditions the nucleic acid sample with a fourth set of amplification primers that amplify a fourth region in the sample nucleic acid allowing for a determination of the amount of digested or undigested nucleic acid, as an indicator of digestion efficiency. In some cases, the fourth region comprises one or more loci present in both fetal nucleic acid and maternal nucleic acid and unmethylated in both fetal nucleic acid and maternal nucleic acid.

In some embodiments, a method further comprises contacting under amplification conditions the nucleic acid sample with a predetermined copy number of one or more first competitor oligonucleotides that compete with the first region for hybridization of primers of the first amplification primer set. In some embodiments, a method further comprises contacting under amplification conditions the nucleic acid sample with a predetermined copy number of one or more first competitor oligonucleotides that compete with the target chromosome for hybridization of primers of the first amplification primer set.

In some embodiments, a method further comprises contacting under amplification conditions the nucleic acid sample with a predetermined copy number of one or more second competitor oligonucleotides that compete with the second region for hybridization of primers of the second amplification primer set. In some embodiments, a method further comprises contacting under amplification conditions the nucleic acid sample with a predetermined copy number of one or more second competitor oligonucleotides that compete with the reference chromosome for hybridization of primers of the second amplification primer set.

In some embodiments, a method further comprises contacting under amplification conditions the nucleic acid sample with a predetermined copy number of one or more third competitor oligonucleotides that compete with the third region for hybridization of primers of the third amplification primer set.

In some embodiments, a method further comprises contacting under amplification conditions the nucleic acid sample with a predetermined copy number of one or more fourth competitor oligonucleotides that compete with the fourth region for hybridization of primers of the fourth amplification primer set.

In some embodiments, the amount of fetal nucleic acid determined is the copy number of fetal nucleic acid based on the amount of competitor oligonucleotide used. In some embodiments, the amount of fetal nucleic acid determined is the copy number of fetal nucleic acid based on a quantification of sequence reads.

In some embodiments, the sample nucleic acid is extracellular nucleic acid. In some cases, the nucleic acid sample is obtained from a pregnant female subject. In some cases, the subject is human. In some embodiments, the sample nucleic acid is from plasma or serum.

In some embodiments, two or more independent loci in the first region are assayed. In some embodiments, two or more independent loci in the target chromosome are assayed. In some embodiments, two or more independent loci in the reference chromosome are assayed. In some embodiments, the target chromosome is chromosome 13. In some embodiments, the target chromosome is chromosome 18. In some embodiments, the target chromosome is chromosome 21.

In some embodiments, the amount of fetal nucleic acid is substantially equal to the amount of fetal nucleic acid determined using a mass spectrometry method. In some embodiments, the amount of fetal nucleic acid is determined with an R² value of 0.97 or greater when compared to an amount of fetal nucleic acid determined using a mass spectrometry method. In some embodiments, the copy number of fetal nucleic acid is substantially equal to the copy number of fetal nucleic acid determined using a mass spectrometry method. In some embodiments, the copy number of fetal nucleic acid is determined with an R² value of 0.97 or greater when compared to a copy number of fetal nucleic acid determined using a mass spectrometry method.

Also provided, in some aspects, are methods for determining fetal fraction in a sample comprising (a) enriching a sample nucleic acid for a plurality of polymorphic nucleic acid targets, which sample nucleic acid comprises fetal nucleic acid and maternal nucleic acid; (b) obtaining nucleotide sequences for some or all of the nucleic acid targets by a sequencing process; (c) analyzing the nucleotide sequences of (b); and (d) determining fetal fraction based on the analysis of (c), where the polymorphic nucleic acid targets and number thereof result in at least five polymorphic nucleic acid targets being informative for determining the fetal fraction for at least 90% of samples.

In some embodiments, the enriching comprises amplifying the plurality of polymorphic nucleic acid targets. In some cases, the enriching comprises generating amplification products in an amplification reaction, and sometimes the amplification reaction is performed in a single vessel.

In some embodiments, the maternal genotype and the paternal genotype at each of the polymorphic nucleic acid targets are not known prior to (a). In some embodiments, polymorphic nucleic acid targets having a minor allele population frequency of about 40% or more are selected.

In some embodiments, a method comprises determining an allele frequency in the sample for each of the polymorphic nucleic acid targets. In some embodiments, determining which polymorphic nucleic acid targets are informative comprises identifying informative genotypes by comparing each allele frequency to one or more fixed cutoff frequencies. In some cases, the fixed cutoff for identifying informative genotypes from non-informative homozygotes is about a 2% or greater shift in allele frequency and sometimes is a 1% or greater shift in allele frequency. In some cases, the fixed cutoff for identifying informative genotypes from non-informative heterozygotes is about a 50% or greater shift in allele frequency and sometimes is a 25% or greater shift in allele frequency. In some embodiments, determining which polymorphic nucleic acid targets are informative comprises identifying informative genotypes by comparing each allele frequency to one or more target-specific cutoff frequencies. In some cases, the one or more target-specific cutoff frequencies are determined for each polymorphic nucleic acid target. In some cases, each target-specific cutoff frequency is determined based on the allele frequency variance for the corresponding polymorphic nucleic acid target.

In some embodiments, a method comprises determining an allele frequency mean. In some cases, fetal fraction is determined based, in part, on the allele frequency mean. In some embodiments, the fetal genotype at one or more informative polymorphic nucleic acid targets is heterozygous. In some embodiments, the fetal genotype at one or more informative polymorphic nucleic acid targets is homozygous. In some embodiments, fetal fraction is determined with a coefficient of variance (CV) of 0.20 or less. In some cases, fetal fraction is determined with a coefficient of variance (CV) of 0.10 or less, and sometimes fetal fraction is determined with a coefficient of variance (CV) of 0.05 or less.

In some embodiments, the polymorphic nucleic acid targets each comprise at least one single nucleotide polymorphism (SNP). In some cases, the SNPs are selected from: rs10413687, rs10949838, rs1115649, rs11207002, rs11632601, rs11971741, rs12660563, rs13155942, rs1444647, rs1572801, rs17773922, rs1797700, rs1921681, rs1958312, rs196008, rs2001778, rs2323659, rs2427099, rs243992, rs251344, rs254264, rs2827530, rs290387, rs321949, rs348971, rs390316, rs3944117, rs425002, rs432586, rs444016, rs4453265, rs447247, rs4745577, rs484312, rs499946, rs500090, rs500399, rs505349, rs505662, rs516084, rs517316, rs517914, rs522810, rs531423, rs537330, rs539344, rs551372, rs567681, rs585487, rs600933, rs619208, rs622994, rs639298, rs642449, rs6700732, rs677866, rs683922, rs686851, rs6941942, rs7045684, rs7176924, rs7525374, rs870429, rs949312, rs9563831, rs970022, rs985462, rs1005241, rs1006101, rs10745725, rs10776856, rs10790342, rs11076499, rs11103233, rs11133637, rs11974817, rs12102203, rs12261, rs12460763, rs12543040, rs12695642, rs13137088, rs13139573, rs1327501, rs13438255, rs1360258, rs1421062, rs1432515, rs1452396, rs1518040, rs16853186, rs1712497, rs1792205, rs1863452, rs1991899, rs2022958, rs2099875, rs2108825, rs2132237, rs2195979, rs2248173, rs2250246, rs2268697, rs2270893, rs244887, rs2736966, rs2851428, rs2906237, rs2929724, rs3742257, rs3764584, rs3814332, rs4131376, rs4363444, rs4461567, rs4467511, rs4559013, rs4714802, rs4775899, rs4817609, rs488446, rs4950877, rs530913, rs6020434, rs6442703, rs6487229, rs6537064, rs654065, rs6576533, rs6661105, rs669161, rs6703320, rs675828, rs6814242, rs6989344, rs7120590, rs7131676, rs7214164, rs747583, rs768255, rs768708, rs7828904, rs7899772, rs7900911, rs7925270, rs7975781, rs8111589, rs849084, rs873870, rs9386151, rs9504197, rs9690525, and rs9909561.

In some cases, the SNPs are selected from: rs10413687, rs10949838, rs1115649, rs11207002, rs11632601, rs11971741, rs12660563, rs13155942, rs1444647, rs1572801, rs17773922, rs1797700, rs1921681, rs1958312, rs196008, rs2001778, rs2323659, rs2427099, rs243992, rs251344, rs254264, rs2827530, rs290387, rs321949, rs348971, rs390316, rs3944117, rs425002, rs432586, rs444016, rs4453265, rs447247, rs4745577, rs484312, rs499946, rs500090, rs500399, rs505349, rs505662, rs516084, rs517316, rs517914, rs522810, rs531423, rs537330, rs539344, rs551372, rs567681, rs585487, rs600933, rs619208, rs622994, rs639298, rs642449, rs6700732, rs677866, rs683922, rs686851, rs6941942, rs7045684, rs7176924, rs7525374, rs870429, rs949312, rs9563831, rs970022, and rs985462.

In some cases, the SNPs are selected from: rs1005241, rs1006101, rs10745725, rs10776856, rs10790342, rs11076499, rs11103233, rs11133637, rs11974817, rs12102203, rs12261, rs12460763, rs12543040, rs12695642, rs13137088, rs13139573, rs1327501, rs13438255, rs1360258, rs1421062, rs1432515, rs1452396, rs1518040, rs16853186, rs1712497, rs1792205, rs1863452, rs1991899, rs2022958, rs2099875, rs2108825, rs2132237, rs2195979, rs2248173, rs2250246, rs2268697, rs2270893, rs244887, rs2736966, rs2851428, rs2906237, rs2929724, rs3742257, rs3764584, rs3814332, rs4131376, rs4363444, rs4461567, rs4467511, rs4559013, rs4714802, rs4775899, rs4817609, rs488446, rs4950877, rs530913, rs6020434, rs6442703, rs6487229, rs6537064, rs654065, rs6576533, rs6661105, rs669161, rs6703320, rs675828, rs6814242, rs6989344, rs7120590, rs7131676, rs7214164, rs747583, rs768255, rs768708, rs7828904, rs7899772, rs7900911, rs7925270, rs7975781, rs8111589, rs849084, rs873870, rs9386151, rs9504197, rs9690525, and rs9909561.

The polymorphic targets can comprise one or more of any of the single nucleotide polymorphisms (SNPs) listed above and any combination thereof.

In some embodiments, the polymorphic nucleic acid targets and number thereof result in at least five polymorphic nucleic acid targets being informative for determining the fetal fraction for at least 95% of samples. In some cases, the polymorphic nucleic acid targets and number thereof result in at least five polymorphic nucleic acid targets being informative for determining the fetal fraction for at least 99% of samples. In some cases, the polymorphic nucleic acid targets and number thereof result in at least ten polymorphic nucleic acid targets being informative for determining the fetal fraction for at least 90% of samples. In some cases, the polymorphic nucleic acid targets and number thereof result in at least ten polymorphic nucleic acid targets being informative for determining the fetal fraction for at least 95% of samples. In some cases, the polymorphic nucleic acid targets and number thereof result in at least ten polymorphic nucleic acid targets being informative for determining the fetal fraction for at least 99% of samples. Sometimes, 10 or more polymorphic nucleic acid targets are enriched, sometimes 50 or more polymorphic nucleic acid targets are enriched, sometimes 100 or more polymorphic nucleic acid targets are enriched, and sometimes 500 or more polymorphic nucleic acid targets are enriched. Sometimes, about 40 to about 100 polymorphic nucleic acid targets are enriched.

In some embodiments, the sequencing process comprises a sequencing by synthesis method. In some cases, the sequencing by synthesis method comprises a plurality of synthesis cycles. Sometimes, the sequencing by synthesis method comprises about 36 cycles and sometimes the sequencing by synthesis method comprises about 27 cycles. In some embodiments, the sequencing process comprises a sequencing by ligation method. In some embodiments, the sequencing process comprises a single molecule sequencing method.

In some embodiments, the sequencing process comprises sequencing a plurality of samples in a single compartment. In some cases, the fetal fraction is determined for 10 or more samples. In some cases, the fetal fraction is determined for 100 or more samples. In some cases, the fetal fraction is determined for 1000 or more samples.

In some embodiments, the sample nucleic acid is cell-free DNA. In some embodiments, the sample nucleic acid is obtained from a pregnant female subject. In some cases, the subject is human. In some cases, the sample nucleic acid is from plasma or serum.

Certain embodiments are described further in the following description, examples, claims and drawings.

### BRIEF DESCRIPTION OF THE DRAWINGS

The drawings illustrate embodiments of the technology herein and are not limiting. For clarity and ease of illustration, the drawings are not made to scale and, in some instances, various aspects may be shown exaggerated or enlarged to facilitate an understanding of particular embodiments.

FIG. 1 shows the design of the recombinant MBD-Fc protein used to separate differentially methylated DNA.

FIG. 2 shows the methyl-CpG-binding, antibody-like protein has a high affinity and high avidity to its “antigen”, which is preferably DNA that is methylated at CpG di-nucleotides.

FIG. 3 shows the methyl binding domain of MBD-FC binds all DNA molecules regardless of their methylation status. The strength of this protein/DNA interaction is defined by the level of DNA methylation. After binding genomic DNA, eluate solutions of increasing salt concentrations can be used to fractionate non-methylated and methylated DNA allowing for a controlled separation.

FIG. 4 shows the experiment used to identify differentially methylated DNA from a fetus and mother using the recombinant MBD-Fc protein and a microarray.

FIG. 5 shows typical results generated by Sequenom® EpiTYPER™ method, which was used to validate the results generated from the experiment illustrated in FIG. 4.

FIG. 6 shows the correlation between the log ratios derived from microarray analysis (x axis) and methylation differences obtained by EpiTYPER™ analysis (y axis). Each data point represents the average for one region across all measured samples. The microarray analysis is comparative in nature because the highly methylated fraction of the maternal DNA is hybridized together with the highly methylated fraction of placenta DNA. Positive values indicate higher methylation of the placenta samples. In mass spectrometry each samples is measured individually. The difference in methylation was calculated by subtracting the maternal methylation values from the placenta methylation value. To compare the results with the microarray data the average of the differences for all maternal/placenta DNA pairs was calculated. Figure discloses SEQ ID NOS 387 and 388, respectively, in order of appearance.

FIG. 7 shows a correlation between microarray and EpiTYPER™ results.

FIG. 8 shows the correlation between the number of gDNA molecules that were expected and the number of molecules measured by competitive PCR in combination with mass spectrometry analysis. In this experiment, DNA derived from whole blood (black plus signs) was used and commercially available fully methylated DNA (red crosses) was used in a 90 to 10 ratio. The MBD-FC fusion protein was used to separate the non-methylated and the methylated fraction of DNA. Each fraction was subject to competitive PCR analysis with mass spectrometry readout. The method has been described earlier for the analysis of copy number variations and is commercially available for gene expression analysis. The approach allows absolute quantification of DNA molecules with the help of a synthetic oligonucleotides of know concentration. In this experiment the MGMT locus was targeted, which was not methylated in the whole blood sample used here. Using an input of 300 total gDNA copies, 270 copies of non-methylated DNA and 30 copies of methylated DNA was expected. The measured copy numbers are largely in agreement with the expected values. The data point at 600 copies of input DNA indicates a bias in the reaction and shows that this initial proof of concept experiment needs to be followed up with more development work, before the assay can be used. However, this initial data indicates the feasibility of the approach for capturing and quantifying of a few copies of methylated DNA in the presence of an excess of unmethylated DNA species.

FIG. 9A-9L show bar graph plots of the methylation differences obtained from the microarray analysis (dark bars) and the mass spectrometry analysis (light grey bars) with respect to their genomic location. For each of the 85 regions that were identified to be differentially methylated by microarray an individual plot is provided. The x axis for each plot shows the chromosomal position of the region. The y axis depicts the log ration (in case of the microarrays) and the methylation differences (in case of the mass spectrometry results). For the microarrays each hybridization probe in the area is shown as a single black (or dark grey) bar. For the mass spectrometry results each CpG site, is shown as a light grey bar. Bars showing values greater than zero indicate higher DNA methylation in the placenta samples compared to the maternal DNA. For some genes the differences are small (i.e. RB1 or DSCR6) but still statistically significant. Those regions would be less suitable for a fetal DNA enrichment strategy.

FIG. 10 shows one embodiment of the Fetal Quantifier Method. Maternal nucleic acid is selectively digested and the remaining fetal nucleic acid is quantified using a competitor of known concentration. In this schema, the analyte is separated and quantified by a mass spectrometer.

FIG. 11 shows one embodiment of the Methylation-Based Fetal Diagnostic Method. Maternal nucleic acid is selectively digested and the remaining fetal nucleic acid is quantified for three different chromosomes (13, 18 and 21). Parts 2 and 3 of the Figure illustrate the size distribution of the nucleic acid in the sample before and after digestion. The amplification reactions can be size-specific (e.g., greater than 100 base pair amplicons) such that they favor the longer, non-digested fetal nucleic acid over the digested maternal nucleic acid, thereby further enriching the fetal nucleic acid. The spectra at the bottom of the Figure show an increased amount of chromosome 21 fetal nucleic acid indicative of trisomy 21.

FIG. 12 shows the total number of amplifiable genomic copies from four different DNA samples isolated from the blood of non-pregnant women. Each sample was diluted to contain approximately 2500, 1250, 625 or 313 copies per reaction. Each measurement was obtained by taking the mean DNA/competitor ratio obtained from two total copy number assays (ALB and RNAseP in Table X). As FIG. 12 shows, the total copy number is accurate and stable across the different samples, thus validating the usefulness of the competitor-based approach.

FIGS. 13A and 13B show a model system that was created that contained a constant number of maternal non-methylated DNA with varying amounts of male placental methylated DNA spiked-in. The samples were spiked with male placental amounts ranging from approximately 0 to 25% relative to the maternal non-methylated DNA. The fraction of placental DNA was calculated using the ratios obtained from the methylation assays (FIG. 13A) and the Y-chromosome marker (FIG. 13B) as compared to the total copy number assay. The methylation and Y-chromosome markers are provided in Table X.

FIGS. 14A and 14B show the results of the total copy number assay from plasma samples. In FIG. 14A, the copy number for each sample is shown. Two samples (no 25 and 26) have a significantly higher total copy number than all the other samples. A mean of approximately 1300 amplifiable copies/ml plasma was obtained (range 766-2055). FIG. 14B shows a box-and-whisker plot of the given values, summarizing the results.

FIGS. 15A and 15B show the amount (or copy numbers) of fetal nucleic acid from 33 different plasma samples taken from pregnant women with male fetuses plotted. The copy numbers obtained were calculated using the methylation markers and the Y-chromosome-specific markers using the assays provided in Table X. As can be seen in FIG. 15B, the box-and-whisker plot of the given values indicated minimal difference between the two different measurements, thus validating the accuracy and stability of the method.

FIG. 16 shows a paired correlation between the results obtained using the methylation markers versus the Y-chromosome marker from FIG. 15A.

FIG. 17 shows the digestion efficiency of the restriction enzymes using the ratio of digestion for the control versus the competitor and comparing this value to the mean total copy number assays. Apart from sample 26 all reactions indicate the efficiency to be above about 99%.

FIG. 18 provides a specific method for calculating fetal DNA fraction (or concentration) in a sample using the Y-chromosome-specific markers for male pregnancies and the mean of the methylated fraction for all pregnancies (regardless of fetal sex).

FIG. 19 provides a specific method for calculating fetal DNA fraction (or concentration) in a sample without the Y-chromosome-specific markers. Instead, only the Assays for Methylation Quantification were used to determine the concentration of fetal DNA.

FIG. 20 shows a power calculation t-test for a simulated trisomy 21 diagnosis using the methods of the technology herein. The Figure shows the relationship between the coefficient of variation (CV) on the x-axis and the power to discriminate the assay populations using a simple t-test (y-axis). The data indicates that in 99% of all cases, one can discriminate the two population (euploid vs. aneuploid) on a significance level of 0.001 provided a CV of 5% or less.

FIG. 21 shows a scheme for ligating a PCR amplicon with Illumina sequencing adaptors.

FIG. 22 shows a modified ligation scheme.

FIG. 23 shows a comparison of copy numbers of individual markers determined by a fetal quantification assay using MPSS (FQA Sequencing; x-axis) with those obtained by a fetal quantification assay using MASSARRAY (FQA MA; y-axis). The results from both methods were highly correlated (R²>0.97). In some cases, platform-specific allele bias resulted in slight copy number differences and slopes of the linear fit which deviated from 1.

FIG. 24 shows a comparison of mean copy numbers for each of the marker groups determined by a fetal quantification assay using MPSS (FQA Sequencing; x-axis) with those obtained by a fetal quantification assay using MASSARRAY (FQA MA; y-axis).

FIG. 25 shows a comparison of fetal fractions derived from either methylation (left) or Y-chromosome markers determined by a fetal quantification assay using MPSS (FQA Sequencing; x-axis) with those obtained by a fetal quantification assay using MASSARRAY (FQA MA; y-axis).

FIG. 26 shows an example of a likelihood chart for an informative fetal/maternal genotype combination.

FIG. 27 illustrates a possible distribution of maternal and paternal alleles.

FIG. 28 illustrates a method for calculating fetal fraction by MPSS.

FIG. 29 illustrates a scheme for multiplexed amplicon library generation and sequencing.

FIG. 30 shows allele frequencies per SNP for a particular sample.

FIG. 31 shows allele frequencies per SNP for a particular sample.

FIG. 32 shows allele frequencies per sample for a collection of 46 samples.

FIG. 33 shows allele frequencies per sample (folded on 0.5) for a collection of 46 samples.

FIG. 34 shows fetal fraction values calculated from informative genotypes for each sample.

FIG. 35 shows a correlation plot for SNP-based fetal fraction estimates versus methylation-based fetal fraction estimates.

FIG. 36 shows a comparison of informative genotype measurements at varying sequencing coverage.

FIG. 37 shows probabilities of the number of informative SNPs for each of the selected thresholds (1-6 informative SNPs) at increasing numbers of total SNPs assayed.

### DEFINITIONS

The term “pregnancy-associated disorder,” as used in this application, refers to any condition or disease that may affect a pregnant woman, the fetus, or both the woman and the fetus. Such a condition or disease may manifest its symptoms during a limited time period, e.g., during pregnancy or delivery, or may last the entire life span of the fetus following its birth. Some examples of a pregnancy-associated disorder include ectopic pregnancy, preeclampsia, preterm labor, RhD incompatibility, fetal chromosomal abnormalities such as trisomy 21, and genetically inherited fetal disorders such as cystic fibrosis, beta-thalassemia or other monogenic disorders. The compositions and processes described herein are particularly useful for diagnosis, prognosis and monitoring of pregnancy-associated disorders associated with quantitative abnormalities of fetal DNA in maternal plasma/serum, including but not limited to, preeclampsia (Lo et al., Clin. Chem. 45:184-188, 1999 and Zhong et al., Am. J. Obstet. Gynecol. 184:414-419, 2001), fetal trisomy (Lo et al., Clin. Chem. 45:1747-1751, 1999 and Zhong et al., Prenat. Diagn. 20:795-798, 2000) and hyperemesis gravidarum (Sekizawa et al., Clin. Chem. 47:2164-2165, 2001). For example, an elevated level of fetal nucleic acid in maternal blood (as compared to a normal pregnancy or pregnancies) may be indicative of a preeclamptic pregnancy. Further, the ability to enrich fetal nucleic from a maternal sample may prove particularly useful for the noninvasive prenatal diagnosis of autosomal recessive diseases such as the case when a mother and father share an identical disease causing mutation, an occurrence previously perceived as a challenge for maternal plasma-based non-trisomy prenatal diagnosis.

The term “chromosomal abnormality” or “aneuploidy” as used herein refers to a deviation between the structure of the subject chromosome and a normal homologous chromosome. The term “normal” refers to the predominate karyotype or banding pattern found in healthy individuals of a particular species, for example, a euploid genome (in humans, 46XX or 46XY). A chromosomal abnormality can be numerical or structural, and includes but is not limited to aneuploidy, polyploidy, inversion, a trisomy, a monosomy, duplication, deletion, deletion of a part of a chromosome, addition, addition of a part of chromosome, insertion, a fragment of a chromosome, a region of a chromosome, chromosomal rearrangement, and translocation. Chromosomal abnormality may also refer to a state of chromosomal abnormality where a portion of one or more chromosomes is not an exact multiple of the usual haploid number due to, for example, chromosome translocation. Chromosomal translocation (e.g. translocation between chromosome 21 and 14 where some of the 14th chromosome is replaced by extra 21st chromosome) may cause partial trisomy 21. A chromosomal abnormality can be correlated with presence of a pathological condition or with a predisposition to develop a pathological condition. A chromosomal abnormality may be detected by quantitative analysis of nucleic acid.

The terms “nucleic acid” and “nucleic acid molecule” may be used interchangeably throughout the disclosure. The terms refer to nucleic acids of any composition from, such as DNA (e.g., complementary DNA (cDNA), genomic DNA (gDNA) and the like), RNA (e.g., message RNA (mRNA), short inhibitory RNA (siRNA), ribosomal RNA (rRNA), tRNA, microRNA, RNA highly expressed by the fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. For example, the nucleic acids provided in SEQ ID NOs: 1-261 (see Tables 4A-4C) can be in any form useful for conducting processes herein (e.g., linear, circular, supercoiled, single-stranded, double-stranded and the like) or may include variations (e.g., insertions, deletions or substitutions) that do not alter their utility as part of the present technology. A nucleic acid may be, or may be from, a plasmid, phage, autonomously replicating sequence (ARS), centromere, artificial chromosome, chromosome, or other nucleic acid able to replicate or be replicated in vitro or in a host cell, a cell, a cell nucleus or cytoplasm of a cell in certain embodiments. A template nucleic acid in some embodiments can be from a single chromosome (e.g., a nucleic acid sample may be from one chromosome of a sample obtained from a diploid organism). Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense”, “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. For RNA, the base cytosine is replaced with uracil. A template nucleic acid may be prepared using a nucleic acid obtained from a subject as a template.

A “nucleic acid comprising one or more CpG sites” or a “CpG-containing genomic sequence” as used herein refers to a segment of DNA sequence at a defined location in the genome of an individual such as a human fetus or a pregnant woman. Typically, a “CpG-containing genomic sequence” is at least 15 nucleotides in length and contains at least one cytosine. Preferably, it can be at least 30, 50, 80, 100, 150, 200, 250, or 300 nucleotides in length and contains at least 2, 5, 10, 15, 20, 25, or 30 cytosines. For anyone “CpG-containing genomic sequence” at a given location, e.g., within a region centering around a given genetic locus (see Tables 1A-1C), nucleotide sequence variations may exist from individual to individual and from allele to allele even for the same individual. Typically, such a region centering around a defined genetic locus (e.g., a CpG island) contains the locus as well as upstream and/or downstream sequences. Each of the upstream or downstream sequence (counting from the 5′ or 3′ boundary of the genetic locus, respectively) can be as long as 10 kb, in other cases may be as long as 5 kb, 2 kb, 1 kb, 500 bp, 200 bp, or 100 bp. Furthermore, a “CpG-containing genomic sequence” may encompass a nucleotide sequence transcribed or not transcribed for protein production, and the nucleotide sequence can be an inter-gene sequence, intra-gene sequence, protein-coding sequence, a non protein-coding sequence (such as a transcription promoter), or a combination thereof.

As used herein, a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring, however, for purposes herein, thymine is not considered a methylated nucleotide when present in DNA since thymine is a typical nucleotide base of DNA. Typical nucleoside bases for DNA are thymine, adenine, cytosine and guanine. Typical bases for RNA are uracil, adenine, cytosine and guanine.

Correspondingly a “methylation site” is the location in the target gene nucleic acid region where methylation has, or has the possibility of occurring. For example a location containing CpG is a methylation site where the cytosine may or may not be methylated.

As used herein, a “CpG site” or “methylation site” is a nucleotide within a nucleic acid that is susceptible to methylation either by natural occurring events in vivo or by an event instituted to chemically methylate the nucleotide in vitro.

As used herein, a “methylated nucleic acid molecule” refers to a nucleic acid molecule that contains one or more methylated nucleotides that is/are methylated.

A “CpG island” as used herein describes a segment of DNA sequence that comprises a functionally or structurally deviated CpG density. For example, Yamada et al. (Genome Research 14:247-266, 2004) have described a set of standards for determining a CpG island: it must be at least 400 nucleotides in length, has a greater than 50% GC content, and an OCF/ECF ratio greater than 0.6. Others (Takai et al., Proc. Natl. Acad. Sci. U.S.A. 99:3740-3745, 2002) have defined a CpG island less stringently as a sequence at least 200 nucleotides in length, having a greater than 50% GC content, and an OCF/ECF ratio greater than 0.6.

The term “epigenetic state” or “epigenetic status” as used herein refers to any structural feature at a molecular level of a nucleic acid (e.g., DNA or RNA) other than the primary nucleotide sequence. For instance, the epigenetic state of a genomic DNA may include its secondary or tertiary structure determined or influenced by, e.g., its methylation pattern or its association with cellular proteins.

The term “methylation profile” “methylation state” or “methylation status,” as used herein to describe the state of methylation of a genomic sequence, refers to the characteristics of a DNA segment at a particular genomic locus relevant to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles. The term “methylation” profile” or “methylation status” also refers to the relative or absolute concentration of methylated C or unmethylated C at any particular stretch of residues in a biological sample. For example, if the cytosine (C) residue(s) within a DNA sequence are methylated it may be referred to as “hypermethylated”; whereas if the cytosine (C) residue(s) within a DNA sequence are not methylated it may be referred to as “hypomethylated”. Likewise, if the cytosine (C) residue(s) within a DNA sequence (e.g., fetal nucleic acid) are methylated as compared to another sequence from a different region or from a different individual (e.g., relative to maternal nucleic acid), that sequence is considered hypermethylated compared to the other sequence. Alternatively, if the cytosine (C) residue(s) within a DNA sequence are not methylated as compared to another sequence from a different region or from a different individual (e.g., the mother), that sequence is considered hypomethylated compared to the other sequence. These sequences are said to be “differentially methylated”, and more specifically, when the methylation status differs between mother and fetus, the sequences are considered “differentially methylated maternal and fetal nucleic acid”.

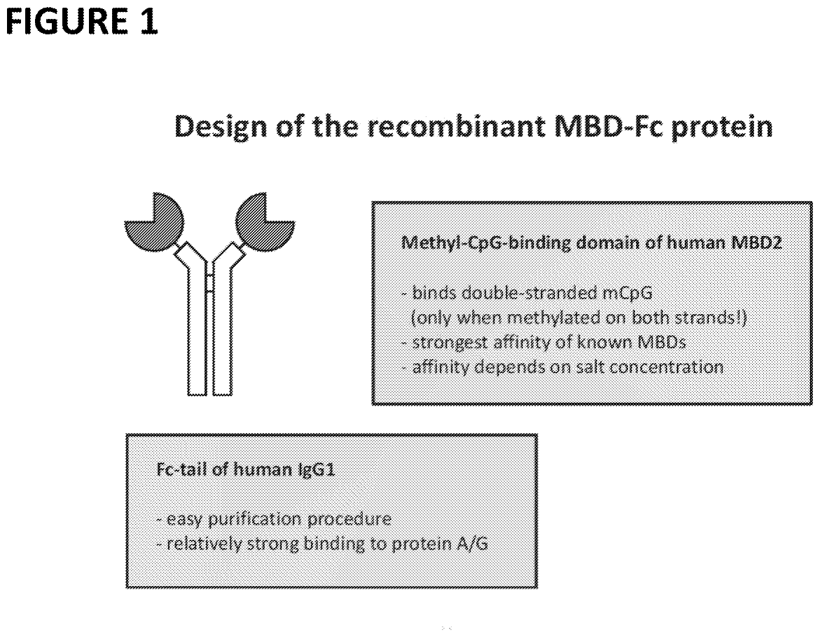

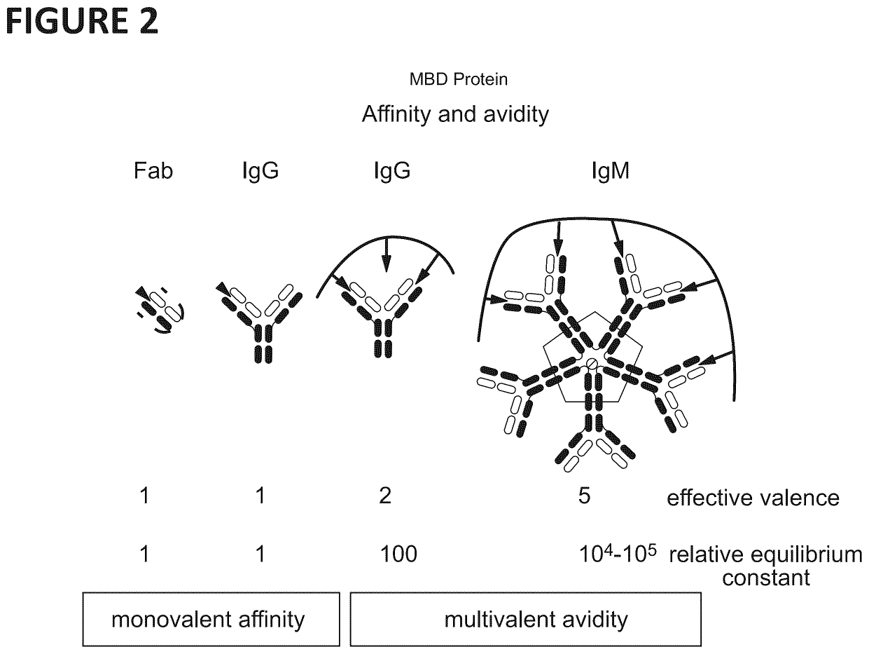

The term “agent that binds to methylated nucleotides” as used herein refers to a substance that is capable of binding to methylated nucleic acid. The agent may be naturally-occurring or synthetic, and may be modified or unmodified. In one embodiment, the agent allows for the separation of different nucleic acid species according to their respective methylation states. An example of an agent that binds to methylated nucleotides is described in PCT Patent Application No. PCT/EP2005/012707, which published as WO06056480A2 and is hereby incorporated by reference. The described agent is a bifunctional polypeptide comprising the DNA-binding domain of a protein belonging to the family of Methyl-CpG binding proteins (MBDs) and an Fc portion of an antibody (see FIG. 1). The recombinant methyl-CpG-binding, antibody-like protein can preferably bind CpG methylated DNA in an antibody-like manner. That means, the methyl-CpG-binding, antibody-like protein has a high affinity and high avidity to its “antigen”, which is preferably DNA that is methylated at CpG dinucleotides. The agent may also be a multivalent MBD (see FIG. 2).

The term “polymorphism” or “polymorphic nucleic acid target” as used herein refers to a sequence variation within different alleles of the same genomic sequence. A sequence that contains a polymorphism is considered a “polymorphic sequence”. Detection of one or more polymorphisms allows differentiation of different alleles of a single genomic sequence or between two or more individuals. As used herein, the term “polymorphic marker” or “polymorphic sequence” refers to segments of genomic DNA that exhibit heritable variation in a DNA sequence between individuals. Such markers include, but are not limited to, single nucleotide polymorphisms (SNPs), restriction fragment length polymorphisms (RFLPs), short tandem repeats, such as di-, tri- or tetra-nucleotide repeats (STRs), deletions, duplications, and the like. Polymorphic markers according to the present technology can be used to specifically differentiate between a maternal and paternal allele in the enriched fetal nucleic acid sample.

The terms “single nucleotide polymorphism” or “SNP” as used herein refer to the polynucleotide sequence variation present at a single nucleotide residue within different alleles of the same genomic sequence. This variation may occur within the coding region or non-coding region (i.e., in the promoter or intronic region) of a genomic sequence, if the genomic sequence is transcribed during protein production. Detection of one or more SNP allows differentiation of different alleles of a single genomic sequence or between two or more individuals.

The term “allele” as used herein is one of several alternate forms of a gene or non-coding regions of DNA that occupy the same position on a chromosome. The term allele can be used to describe DNA from any organism including but not limited to bacteria, viruses, fungi, protozoa, molds, yeasts, plants, humans, non-humans, animals, and archeabacteria.

The terms “ratio of the alleles” or “allelic ratio” as used herein refer to the ratio of the population of one allele and the population of the other allele in a sample. In some trisomic cases, it is possible that a fetus may be tri-allelic for a particular locus. In such cases, the term “ratio of the alleles” refers to the ratio of the population of any one allele against one of the other alleles, or any one allele against the other two alleles.

The term “non-polymorphism-based quantitative method” as used herein refers to a method for determining the amount of an analyte (e.g., total nucleic acid, Y-chromosome nucleic acid, or fetal nucleic acid) that does not require the use of a polymorphic marker or sequence. Although a polymorphism may be present in the sequence, said polymorphism is not required to quantify the sequence. Examples of non-polymorphism-based quantitative methods include, but are not limited to, RT-PCR, digital PCR, array-based methods, sequencing methods, nanopore-based methods, nucleic acid-bound bead-based counting methods and competitor-based methods where one or more competitors are introduced at a known concentration(s) to determine the amount of one or more analytes. In some embodiments, some of the above exemplary methods (for example, sequencing) may need to be actively modified or designed such that one or more polymorphisms are not interrogated.

As used herein, a “competitor oligonucleotide” or “competitive oligonucleotide” or “competitor” is a nucleic acid polymer that competes with a target nucleotide sequence for hybridization of amplification primers. Often, a competitor has a similar nucleotide sequence as a corresponding target nucleotide sequence. In some cases, a competitor sequence and a corresponding target nucleotide sequence differ by one or more nucleotides. In some cases, a competitor sequence and a corresponding target nucleotide sequence are the same length. In some cases, the competitor optionally has an additional length of nucleotide sequence that is different from the target nucleotide sequence. In some embodiments, a known amount, or copy number, of competitor is used. In some embodiments, two or more competitors are used. In some cases, the two or more competitors possess similar characteristics (e.g. sequence, length, detectable label). In some cases, the two or more competitors possess different characteristics (e.g. sequence, length, detectable label). In some embodiments, one or more competitors are used for a particular region. In some cases, the competitor possesses a characteristic that is unique for each set of competitors for a given region. Often, competitors for different regions possess different characteristics.

A competitor oligonucleotide may be composed of naturally occurring and/or non-naturally occurring nucleotides (e.g., labeled nucleotides), or a mixture thereof. Competitor oligonucleotides suitable for use with embodiments described herein, may be synthesized and labeled using known techniques. Competitor oligonucleotides may be chemically synthesized according to any suitable method known, for example, the solid phase phosphoramidite triester method first described by Beaucage and Caruthers, Tetrahedron Letts., 22:1859-1862, 1981, using an automated synthesizer, as described in Needham-VanDevanter et al., Nucleic Acids Res. 12:6159-6168, 1984. Purification of competitor oligonucleotides can be effected by any suitable method known, for example, native acrylamide gel electrophoresis or by anion-exchange high-performance liquid chromatography (HPLC), for example, as described in Pearson and Regnier, J. Chrom., 255:137-149, 1983.

The terms “absolute amount” or “copy number” as used herein refers to the amount or quantity of an analyte (e.g., total nucleic acid or fetal nucleic acid). The present technology provides compositions and processes for determining the absolute amount of fetal nucleic acid in a mixed maternal sample. Absolute amount or copy number represents the number of molecules available for detection, and may be expressed as the genomic equivalents per unit. The term “concentration” refers to the amount or proportion of a substance in a mixture or solution (e.g., the amount of fetal nucleic acid in a maternal sample that comprises a mixture of maternal and fetal nucleic acid). The concentration may be expressed as a percentage, which is used to express how large/small one quantity is, relative to another quantity as a fraction of 100. Platforms for determining the quantity or amount of an analyte (e.g., target nucleic acid) include, but are not limited to, mass spectrometery, digital PCR, sequencing by synthesis platforms (e.g., pyrosequencing), fluorescence spectroscopy and flow cytometry.

The term “sample” as used herein refers to a specimen containing nucleic acid. Examples of samples include, but are not limited to, tissue, bodily fluid (for example, blood, serum, plasma, saliva, urine, tears, peritoneal fluid, ascitic fluid, vaginal secretion, breast fluid, breast milk, lymph fluid, cerebrospinal fluid or mucosa secretion), umbilical cord blood, chorionic villi, amniotic fluid, an embryo, a two-celled embryo, a four-celled embryo, an eight-celled embryo, a 16-celled embryo, a 32-celled embryo, a 64-celled embryo, a 128-celled embryo, a 256-celled embryo, a 512-celled embryo, a 1024-celled embryo, embryonic tissues, lymph fluid, cerebrospinal fluid, mucosa secretion, or other body exudate, fecal matter, an individual cell or extract of the such sources that contain the nucleic acid of the same, and subcellular structures such as mitochondria, using protocols well established within the art.

Fetal DNA can be obtained from sources including but not limited to maternal blood, maternal serum, maternal plasma, fetal cells, umbilical cord blood, chorionic villi, amniotic fluid, urine, saliva, lung lavage, cells or tissues.

The term “blood” as used herein refers to a blood sample or preparation from a pregnant woman or a woman being tested for possible pregnancy. The term encompasses whole blood or any fractions of blood, such as serum and plasma as conventionally defined.

The term “bisulfite” as used herein encompasses all types of bisulfites, such as sodium bisulfite, that are capable of chemically converting a cytosine (C) to a uracil (U) without chemically modifying a methylated cytosine and therefore can be used to differentially modify a DNA sequence based on the methylation status of the DNA.

As used herein, a reagent or agent that “differentially modifies” methylated or non-methylated DNA encompasses any reagent that modifies methylated and/or unmethylated DNA in a process through which distinguishable products result from methylated and non-methylated DNA, thereby allowing the identification of the DNA methylation status. Such processes may include, but are not limited to, chemical reactions (such as a C→U conversion by bisulfite) and enzymatic treatment (such as cleavage by a methylation-dependent endonuclease). Thus, an enzyme that preferentially cleaves or digests methylated DNA is one capable of cleaving or digesting a DNA molecule at a much higher efficiency when the DNA is methylated, whereas an enzyme that preferentially cleaves or digests unmethylated DNA exhibits a significantly higher efficiency when the DNA is not methylated.

The terms “non-bisulfite-based method” and “non-bisulfite-based quantitative method” as used herein refer to any method for quantifying methylated or non-methylated nucleic acid that does not require the use of bisulfite. The terms also refer to methods for preparing a nucleic acid to be quantified that do not require bisulfite treatment. Examples of non-bisulfite-based methods include, but are not limited to, methods for digesting nucleic acid using one or more methylation sensitive enzymes and methods for separating nucleic acid using agents that bind nucleic acid based on methylation status.

The terms “methyl-sensitive enzymes” and “methylation sensitive restriction enzymes” are DNA restriction endonucleases that are dependent on the methylation state of their DNA recognition site for activity. For example, there are methyl-sensitive enzymes that cleave or digest at their DNA recognition sequence only if it is not methylated. Thus, an unmethylated DNA sample will be cut into smaller fragments than a methylated DNA sample. Similarly, a hypermethylated DNA sample will not be cleaved. In contrast, there are methyl-sensitive enzymes that cleave at their DNA recognition sequence only if it is methylated. As used herein, the terms “cleave”, “cut” and “digest” are used interchangeably.

The term “target nucleic acid” as used herein refers to a nucleic acid examined using the methods disclosed herein to determine if the nucleic acid is part of a pregnancy-related disorder or chromosomal abnormality. For example, a target nucleic acid from chromosome 21 could be examined using the methods of the technology herein to detect Down's Syndrome.

The term “control nucleic acid” as used herein refers to a nucleic acid used as a reference nucleic acid according to the methods disclosed herein to determine if the nucleic acid is part of a chromosomal abnormality. For example, a control nucleic acid from a chromosome other than chromosome 21 (herein referred to as a “reference chromosome”) could be as a reference sequence to detect Down's Syndrome. In some embodiments, the control sequence has a known or predetermined quantity.

The term “sequence-specific” or “locus-specific method” as used herein refers to a method that interrogates (for example, quantifies) nucleic acid at a specific location (or locus) in the genome based on the sequence composition. Sequence-specific or locus-specific methods allow for the quantification of specific regions or chromosomes.

The term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

In this application, the terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), where the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, .gamma.-carboxyglutamate, and O-phosphoserine.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Primers” as used herein refer to oligonucleotides that can be used in an amplification method, such as a polymerase chain reaction (PCR), to amplify a nucleotide sequence based on the polynucleotide sequence corresponding to a particular genomic sequence, e.g., one located within the CpG island CGI137, PDE9A, or CGI009 on chromosome 21, in various methylation status. At least one of the PCR primers for amplification of a polynucleotide sequence is sequence-specific for the sequence.

The term “template” refers to any nucleic acid molecule that can be used for amplification in the technology herein. RNA or DNA that is not naturally double stranded can be made into double stranded DNA so as to be used as template DNA. Any double stranded DNA or preparation containing multiple, different double stranded DNA molecules can be used as template DNA to amplify a locus or loci of interest contained in the template DNA.

The term “amplification reaction” as used herein refers to a process for copying nucleic acid one or more times. In embodiments, the method of amplification includes but is not limited to polymerase chain reaction, self-sustained sequence reaction, ligase chain reaction, rapid amplification of cDNA ends, polymerase chain reaction and ligase chain reaction, Q-beta phage amplification, strand displacement amplification, or splice overlap extension polymerase chain reaction. In some embodiments, a single molecule of nucleic acid is amplified, for example, by digital PCR.

The term “sensitivity” as used herein refers to the number of true positives divided by the number of true positives plus the number of false negatives, where sensitivity (sens) may be within the range of 0≤sens≤1. Ideally, method embodiments herein have the number of false negatives equaling zero or close to equaling zero, so that no subject is wrongly identified as not having at least one chromosome abnormality or other genetic disorder when they indeed have at least one chromosome abnormality or other genetic disorder. Conversely, an assessment often is made of the ability of a prediction algorithm to classify negatives correctly, a complementary measurement to sensitivity. The term “specificity” as used herein refers to the number of true negatives divided by the number of true negatives plus the number of false positives, where sensitivity (spec) may be within the range of 0 s spec s 1. Ideally, methods embodiments herein have the number of false positives equaling zero or close to equaling zero, so that no subject wrongly identified as having at least one chromosome abnormality other genetic disorder when they do not have the chromosome abnormality other genetic disorder being assessed. Hence, a method that has sensitivity and specificity equaling one, or 100%, sometimes is selected.

One or more prediction algorithms may be used to determine significance or give meaning to the detection data collected under variable conditions that may be weighed independently of or dependently on each other. The term “variable” as used herein refers to a factor, quantity, or function of an algorithm that has a value or set of values. For example, a variable may be the design of a set of amplified nucleic acid species, the number of sets of amplified nucleic acid species, percent fetal genetic contribution tested, percent maternal genetic contribution tested, type of chromosome abnormality assayed, type of genetic disorder assayed, type of sex-linked abnormalities assayed, the age of the mother and the like. The term “independent” as used herein refers to not being influenced or not being controlled by another. The term “dependent” as used herein refers to being influenced or controlled by another. For example, a particular chromosome and a trisomy event occurring for that particular chromosome that results in a viable being are variables that are dependent upon each other.

One of skill in the art may use any type of method or prediction algorithm to give significance to the data of the present technology within an acceptable sensitivity and/or specificity. For example, prediction algorithms such as Chi-squared test, z-test, t-test, ANOVA (analysis of variance), regression analysis, neural nets, fuzzy logic, Hidden Markov Models, multiple model state estimation, and the like may be used. One or more methods or prediction algorithms may be determined to give significance to the data having different independent and/or dependent variables of the present technology. And one or more methods or prediction algorithms may be determined not to give significance to the data having different independent and/or dependent variables of the present technology. One may design or change parameters of the different variables of methods described herein based on results of one or more prediction algorithms (e.g., number of sets analyzed, types of nucleotide species in each set). For example, applying the Chi-squared test to detection data may suggest that specific ranges of maternal age are correlated to a higher likelihood of having an offspring with a specific chromosome abnormality, hence the variable of maternal age may be weighed differently verses being weighed the same as other variables.

In certain embodiments, several algorithms may be chosen to be tested. These algorithms can be trained with raw data. For each new raw data sample, the trained algorithms will assign a classification to that sample (i.e. trisomy or normal). Based on the classifications of the new raw data samples, the trained algorithms' performance may be assessed based on sensitivity and specificity. Finally, an algorithm with the highest sensitivity and/or specificity or combination thereof may be identified.

### DETAILED DESCRIPTION

The presence of fetal nucleic acid in maternal plasma was first reported in 1997 and offers the possibility for non-invasive prenatal diagnosis simply through the analysis of a maternal blood sample (Lo et al., Lancet 350:485-487, 1997). To date, numerous potential clinical applications have been developed. In particular, quantitative abnormalities of fetal nucleic acid, for example DNA, concentrations in maternal plasma have been found to be associated with a number of pregnancy-associated disorders, including preeclampsia, preterm labor, antepartum hemorrhage, invasive placentation, fetal Down syndrome, and other fetal chromosomal aneuploidies. Hence, fetal nucleic acid analysis in maternal plasma represents a powerful mechanism for the monitoring of fetomaternal well-being.

However, fetal DNA co-exists with background maternal DNA in maternal plasma. Hence, most reported applications have relied on the detection of Y-chromosome sequences as these are most conveniently distinguishable from maternal DNA. Such an approach limits the applicability of the existing assays to only 50% of all pregnancies, namely those with male fetuses. Thus, there is much need for the development of sex-independent compositions and methods for enriching and analyzing fetal nucleic acid from a maternal sample. Also, methods that rely on polymorphic markers to quantify fetal nucleic acid may be susceptible to varying heterozygosity rates across different ethnicities thereby limiting their applicability (e.g., by increasing the number of markers that are needed).

It was previously demonstrated that fetal and maternal DNA can be distinguished by their differences in methylation status (U.S. Pat. No. 6,927,028, which is hereby incorporated by reference). Methylation is an epigenetic phenomenon, which refers to processes that alter a phenotype without involving changes in the DNA sequence. By exploiting the difference in the DNA methylation status between mother and fetus, one can successfully detect and analyze fetal nucleic acid in a background of maternal nucleic acid.

The present inventors provides novel genomic polynucleotides that are differentially methylated between the fetal DNA from the fetus (e.g., from the placenta) and the maternal DNA from the mother, for example from peripheral blood cells. This discovery thus provides a new approach for distinguishing fetal and maternal genomic DNA and new methods for accurately quantifying fetal nucleic which may be used for non-invasive prenatal diagnosis.

### Methodology

Practicing the technology herein utilizes routine techniques in the field of molecular biology. Basic texts disclosing the general methods of use in the technology herein include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange high performance liquid chromatography (HPLC) as described in Pearson & Reanier, J. Chrom. 255: 137-149 (1983).

### Samples

Provided herein are methods and compositions for analyzing nucleic acid. In some embodiments, nucleic acid fragments in a mixture of nucleic acid fragments are analyzed. A mixture of nucleic acids can comprise two or more nucleic acid fragment species having different nucleotide sequences, different fragment lengths, different origins (e.g., genomic origins, fetal vs. maternal origins, cell or tissue origins, sample origins, subject origins, and the like), or combinations thereof.

Nucleic acid or a nucleic acid mixture utilized in methods and apparatuses described herein often is isolated from a sample obtained from a subject. A subject can be any living or non-living organism, including but not limited to a human, a non-human animal, a plant, a bacterium, a fungus or a protist. Any human or non-human animal can be selected, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female (e.g., woman).

Nucleic acid may be isolated from any type of suitable biological specimen or sample. Non-limiting examples of specimens include fluid or tissue from a subject, including, without limitation, umbilical cord blood, chorionic villi, amniotic fluid, cerbrospinal fluid, spinal fluid, lavage fluid (e.g., bronchoalveolar, gastric, peritoneal, ductal, ear, athroscopic), biopsy sample (e.g., from pre-implantation embryo), celocentesis sample, fetal nucleated cells or fetal cellular remnants, washings of female reproductive tract, urine, feces, sputum, saliva, nasal mucous, prostate fluid, lavage, semen, lymphatic fluid, bile, tears, sweat, breast milk, breast fluid, embryonic cells and fetal cells (e.g. placental cells). In some embodiments, a biological sample is a cervical swab from a subject. In some embodiments, a biological sample may be blood and sometimes plasma or serum. As used herein, the term “blood” encompasses whole blood or any fractions of blood, such as serum and plasma as conventionally defined, for example. Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3-40 milliliters) often is collected and can be stored according to standard procedures prior to further preparation. A fluid or tissue sample from which nucleic acid is extracted may be acellular. In some embodiments, a fluid or tissue sample may contain cellular elements or cellular remnants. In some embodiments fetal cells or cancer cells may be included in the sample.

A sample often is heterogeneous, by which is meant that more than one type of nucleic acid species is present in the sample. For example, heterogeneous nucleic acid can include, but is not limited to, (i) fetally derived and maternally derived nucleic acid, (ii) cancer and non-cancer nucleic acid, (iii) pathogen and host nucleic acid, and more generally, (iv) mutated and wild-type nucleic acid. A sample may be heterogeneous because more than one cell type is present, such as a fetal cell and a maternal cell, a cancer and non-cancer cell, or a pathogenic and host cell. In some embodiments, a minority nucleic acid species and a majority nucleic acid species is present.

For prenatal applications of technology described herein, fluid or tissue sample may be collected from a female at a gestational age suitable for testing, or from a female who is being tested for possible pregnancy. Suitable gestational age may vary depending on the prenatal test being performed. In certain embodiments, a pregnant female subject sometimes is in the first trimester of pregnancy, at times in the second trimester of pregnancy, or sometimes in the third trimester of pregnancy. In certain embodiments, a fluid or tissue is collected from a pregnant female between about 1 to about 45 weeks of fetal gestation (e.g., at 1-4, 4-8, 8-12, 12-16, 16-20, 20-24, 24-28, 28-32, 32-36, 36-40 or 40-44 weeks of fetal gestation), and sometimes between about 5 to about 28 weeks of fetal gestation (e.g., at 6, 7, 8, 9, 10, 11, 12, 13, 14, 15, 16, 17, 18, 19, 20, 21, 22, 23, 24, 25, 26 or 27 weeks of fetal gestation).

### Acquisition of Blood Samples and Extraction of DNA

The present technology relates to separating, enriching and analyzing fetal DNA found in maternal blood as a non-invasive means to detect the presence and/or to monitor the progress of a pregnancy-associated condition or disorder. Thus, the first steps of practicing the technology herein are to obtain a blood sample from a pregnant woman and extract DNA from the sample.

### Acquisition of Blood Samples

A blood sample is obtained from a pregnant woman at a gestational age suitable for testing using a method of the present technology. The suitable gestational age may vary depending on the disorder tested, as discussed below. Collection of blood from a woman is performed in accordance with the standard protocol hospitals or clinics generally follow. An appropriate amount of peripheral blood, e.g., typically between 5-50 ml, is collected and may be stored according to standard procedure prior to further preparation. Blood samples may be collected, stored or transported in a manner known to the person of ordinary skill in the art to minimize degradation or the quality of nucleic acid present in the sample.

### Preparation of Blood Samples

The analysis of fetal DNA found in maternal blood according to the present technology may be performed using, e.g., the whole blood, serum, or plasma. The methods for preparing serum or plasma from maternal blood are well known among those of skill in the art. For example, a pregnant woman's blood can be placed in a tube containing EDTA or a specialized commercial product such as Vacutainer SST (Becton Dickinson, Franklin Lakes, N.J.) to prevent blood clotting, and plasma can then be obtained from whole blood through centrifugation. On the other hand, serum may be obtained with or without centrifugation-following blood clotting. If centrifugation is used then it is typically, though not exclusively, conducted at an appropriate speed, e.g., 1,500-3,000 times g. Plasma or serum may be subjected to additional centrifugation steps before being transferred to a fresh tube for DNA extraction.

In addition to the acellular portion of the whole blood, DNA may also be recovered from the cellular fraction, enriched in the buffy coat portion, which can be obtained following centrifugation of a whole blood sample from the woman and removal of the plasma.

### Extraction of DNA

There are numerous known methods for extracting DNA from a biological sample including blood. The general methods of DNA preparation (e.g., described by Sambrook and Russell, Molecular Cloning: A Laboratory Manual 3d ed., 2001) can be followed; various commercially available reagents or kits, such as Qiagen's QIAamp Circulating Nucleic Acid Kit, QiaAmp DNA Mini Kit or QiaAmp DNA Blood Mini Kit (Qiagen, Hilden, Germany), GenomicPrep™ Blood DNA Isolation Kit (Promega, Madison, Wis.), and GFX™ Genomic Blood DNA Purification Kit (Amersham, Piscataway, N.J.), may also be used to obtain DNA from a blood sample from a pregnant woman. Combinations of more than one of these methods may also be used.

In some embodiments, the sample may first be enriched or relatively enriched for fetal nucleic acid by one or more methods. For example, the discrimination of fetal and maternal DNA can be performed using the compositions and processes of the present technology alone or in combination with other discriminating factors. Examples of these factors include, but are not limited to, single nucleotide differences between chromosome X and Y, chromosome Y-specific sequences, polymorphisms located elsewhere in the genome, size differences between fetal and maternal DNA and differences in methylation pattern between maternal and fetal tissues.

Other methods for enriching a sample for a particular species of nucleic acid are described in PCT Patent Application Number PCT/US07/69991, filed May 30, 2007, PCT Patent Application Number PCT/US2007/071232, filed Jun. 15, 2007, U.S. Provisional Application Nos. 60/968,876 and 60/968,878 (assigned to the Applicant), (PCT Patent Application Number PCT/EP05/012707, filed Nov. 28, 2005) which are all hereby incorporated by reference. In certain embodiments, maternal nucleic acid is selectively removed (either partially, substantially, almost completely or completely) from the sample.

### Nucleic Acid Isolation and Processing

Nucleic acid may be derived from one or more sources (e.g., cells, soil, etc.) by methods known in the art. Cell lysis procedures and reagents are known in the art and may generally be performed by chemical, physical, or electrolytic lysis methods. For example, chemical methods generally employ lysing agents to disrupt cells and extract the nucleic acids from the cells, followed by treatment with chaotropic salts. Physical methods such as freeze/thaw followed by grinding, the use of cell presses and the like also are useful. High salt lysis procedures also are commonly used. For example, an alkaline lysis procedure may be utilized. The latter procedure traditionally incorporates the use of phenol-chloroform solutions, and an alternative phenol-chloroform-free procedure involving three solutions can be utilized. In the latter procedures, one solution can contain 15 mM Tris, pH 8.0; 10 mM EDTA and 100 μg/ml Rnase A; a second solution can contain 0.2N NaOH and 1% SDS; and a third solution can contain 3M KOAc, pH 5.5. These procedures can be found in Current Protocols in Molecular Biology, John Wiley & Sons, N.Y., 6.3.1-6.3.6 (1989), incorporated herein in its entirety.

The terms “nucleic acid” and “nucleic acid molecule” are used interchangeably. The terms refer to nucleic acids of any composition form, such as deoxyribonucleic acid (DNA, e.g., complementary DNA (cDNA), genomic DNA (gDNA) and the like), ribonucleic acid (RNA, e.g., message RNA (mRNA), short inhibitory RNA (siRNA), ribosomal RNA (rRNA), transfer RNA (tRNA), microRNA, RNA highly expressed by the fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid may be, or may be from, a plasmid, phage, autonomously replicating sequence (ARS), centromere, artificial chromosome, chromosome, or other nucleic acid able to replicate or be replicated in vitro or in a host cell, a cell, a cell nucleus or cytoplasm of a cell in certain embodiments. A nucleic acid in some embodiments can be from a single chromosome (e.g., a nucleic acid sample may be from one chromosome of a sample obtained from a diploid organism). Nucleic acids also include derivatives, variants and analogs of RNA or DNA synthesized, replicated or amplified from single-stranded (“sense” or “antisense”, “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. For RNA, the base cytosine is replaced with uracil and the sugar 2′ position includes a hydroxyl moiety. A nucleic acid may be prepared using a nucleic acid obtained from a subject as a template.

Nucleic acid may be isolated at a different time point as compared to another nucleic acid, where each of the samples is from the same or a different source. A nucleic acid may be from a nucleic acid library, such as a cDNA or RNA library, for example. A nucleic acid may be a result of nucleic acid purification or isolation and/or amplification of nucleic acid molecules from the sample. Nucleic acid provided for processes described herein may contain nucleic acid from one sample or from two or more samples (e.g., from 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more samples). Nucleic acid can include extracellular nucleic acid in certain embodiments. The term “extracellular nucleic acid” as used herein refers to nucleic acid isolated from a source having substantially no cells and also is referred to as “cell-free” nucleic acid and/or “cell-free circulating” nucleic acid. Extracellular nucleic acid often includes no detectable cells and may contain cellular elements or cellular remnants. Non-limiting examples of acellular sources for extracellular nucleic acid are blood plasma, blood serum and urine. As used herein, the term “obtain cell-free circulating sample nucleic acid” includes obtaining a sample directly (e.g., collecting a sample) or obtaining a sample from another who has collected a sample. Without being limited by theory, extracellular nucleic acid may be a product of cell apoptosis and cell breakdown, which provides basis for extracellular nucleic acid often having a series of lengths across a spectrum (e.g., a “ladder”).

Extracellular nucleic acid can include different nucleic acid species, and therefore is referred to herein as “heterogeneous” in certain embodiments. For example, blood serum or plasma from a person having cancer can include nucleic acid from cancer cells and nucleic acid from non-cancer cells. In another example, blood serum or plasma from a pregnant female can include maternal nucleic acid and fetal nucleic acid. In some instances, fetal nucleic acid sometimes is about 5% to about 50% of the overall nucleic acid (e.g., about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49% of the total nucleic acid is fetal nucleic acid). In some embodiments, the majority of fetal nucleic acid in nucleic acid is of a length of about 500 base pairs or less (e.g., about 80, 85, 90, 91, 92, 93, 94, 95, 96, 97, 98, 99 or 100% of fetal nucleic acid is of a length of about 500 base pairs or less). In some embodiments, the majority of fetal nucleic acid in nucleic acid is of a length of about 250 base pairs or less (e.g., about 80, 85, 90, 91, 92, 93, 94, 95, 96, 97, 98, 99 or 100% of fetal nucleic acid is of a length of about 250 base pairs or less). In some embodiments, the majority of fetal nucleic acid in nucleic acid is of a length of about 200 base pairs or less (e.g., about 80, 85, 90, 91, 92, 93, 94, 95, 96, 97, 98, 99 or 100% of fetal nucleic acid is of a length of about 200 base pairs or less). In some embodiments, the majority of fetal nucleic acid in nucleic acid is of a length of about 150 base pairs or less (e.g., about 80, 85, 90, 91, 92, 93, 94, 95, 96, 97, 98, 99 or 100% of fetal nucleic acid is of a length of about 150 base pairs or less). In some embodiments, the majority of fetal nucleic acid in nucleic acid is of a length of about 100 base pairs or less (e.g., about 80, 85, 90, 91, 92, 93, 94, 95, 96, 97, 98, 99 or 100% of fetal nucleic acid is of a length of about 100 base pairs or less).

Nucleic acid may be provided for conducting methods described herein without processing of the sample(s) containing the nucleic acid, in certain embodiments. In some embodiments, nucleic acid is provided for conducting methods described herein after processing of the sample(s) containing the nucleic acid. For example, a nucleic acid may be extracted, isolated, purified or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. An isolated nucleic acid is provided with fewer non-nucleic acid components (e.g., protein, lipid) than the amount of components present in a source sample. A composition comprising isolated nucleic acid can be about 90%, 91%, 92%, 93%, 94%, 95%, 96%, 97%, 98%, 99% or greater than 99% free of non-nucleic acid components. The term “purified” as used herein refers to nucleic acid provided that contains fewer nucleic acid species than in the sample source from which the nucleic acid is derived. A composition comprising nucleic acid may be about 90%, 91%, 92%, 93%, 94%, 95%, 96%, 97%, 98%, 99% or greater than 99% free of other nucleic acid species. The term “amplified” as used herein refers to subjecting nucleic acid of a sample to a process that linearly or exponentially generates amplicon nucleic acids having the same or substantially the same nucleotide sequence as the nucleotide sequence of the nucleic acid in the sample, or portion thereof.

Nucleic acid also may be processed by subjecting nucleic acid to a method that generates nucleic acid fragments, in certain embodiments, before providing nucleic acid for a process described herein. In some embodiments, nucleic acid subjected to fragmentation or cleavage may have a nominal, average or mean length of about 5 to about 10,000 base pairs, about 100 to about 1,000 base pairs, about 100 to about 500 base pairs, or about 10, 15, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000 or 9000 base pairs. Fragments can be generated by any suitable method known in the art, and the average, mean or nominal length of nucleic acid fragments can be controlled by selecting an appropriate fragment-generating procedure. In certain embodiments, nucleic acid of a relatively shorter length can be utilized to analyze sequences that contain little sequence variation and/or contain relatively large amounts of known nucleotide sequence information. In some embodiments, nucleic acid of a relatively longer length can be utilized to analyze sequences that contain greater sequence variation and/or contain relatively small amounts of nucleotide sequence information.

Nucleic acid fragments may contain overlapping nucleotide sequences, and such overlapping sequences can facilitate construction of a nucleotide sequence of the non-fragmented counterpart nucleic acid, or a portion thereof. For example, one fragment may have subsequences x and y and another fragment may have subsequences y and z, where x, y and z are nucleotide sequences that can be 5 nucleotides in length or greater. Overlap sequence y can be utilized to facilitate construction of the x-y-z nucleotide sequence in nucleic acid from a sample in certain embodiments. Nucleic acid may be partially fragmented (e.g., from an incomplete or terminated specific cleavage reaction) or fully fragmented in certain embodiments.

Nucleic acid can be fragmented by various methods known in the art, which include without limitation, physical, chemical and enzymatic processes. Non-limiting examples of such processes are described in U.S. Patent Application Publication No. 20050112590 (published on May 26, 2005, entitled “Fragmentation-based methods and systems for sequence variation detection and discovery,” naming Van Den Boom et al.). Certain processes can be selected to generate non-specifically cleaved fragments or specifically cleaved fragments. Non-limiting examples of processes that can generate non-specifically cleaved fragment nucleic acid include, without limitation, contacting nucleic acid with apparatus that expose nucleic acid to shearing force (e.g., passing nucleic acid through a syringe needle; use of a French press); exposing nucleic acid to irradiation (e.g., gamma, x-ray, UV irradiation; fragment sizes can be controlled by irradiation intensity); boiling nucleic acid in water (e.g., yields about 500 base pair fragments) and exposing nucleic acid to an acid and base hydrolysis process.

As used herein, “fragmentation” or “cleavage” refers to a procedure or conditions in which a nucleic acid molecule, such as a nucleic acid template gene molecule or amplified product thereof, may be severed into two or more smaller nucleic acid molecules. Such fragmentation or cleavage can be sequence specific, base specific, or nonspecific, and can be accomplished by any of a variety of methods, reagents or conditions, including, for example, chemical, enzymatic, physical fragmentation.

As used herein, “fragments”, “cleavage products”, “cleaved products” or grammatical variants thereof, refers to nucleic acid molecules resultant from a fragmentation or cleavage of a nucleic acid template gene molecule or amplified product thereof. While such fragments or cleaved products can refer to all nucleic acid molecules resultant from a cleavage reaction, typically such fragments or cleaved products refer only to nucleic acid molecules resultant from a fragmentation or cleavage of a nucleic acid template gene molecule or the portion of an amplified product thereof containing the corresponding nucleotide sequence of a nucleic acid template gene molecule. For example, an amplified product can contain one or more nucleotides more than the amplified nucleotide region of a nucleic acid template sequence (e.g., a primer can contain “extra” nucleotides such as a transcriptional initiation sequence, in addition to nucleotides complementary to a nucleic acid template gene molecule, resulting in an amplified product containing “extra” nucleotides or nucleotides not corresponding to the amplified nucleotide region of the nucleic acid template gene molecule). Accordingly, fragments can include fragments arising from portions of amplified nucleic acid molecules containing, at least in part, nucleotide sequence information from or based on the representative nucleic acid template molecule.

As used herein, the term “complementary cleavage reactions” refers to cleavage reactions that are carried out on the same nucleic acid using different cleavage reagents or by altering the cleavage specificity of the same cleavage reagent such that alternate cleavage patterns of the same target or reference nucleic acid or protein are generated. In certain embodiments, nucleic acid may be treated with one or more specific cleavage agents (e.g., 1, 2, 3, 4, 5, 6, 7, 8, 9, 10 or more specific cleavage agents) in one or more reaction vessels (e.g., nucleic acid is treated with each specific cleavage agent in a separate vessel).

Nucleic acid may be specifically cleaved by contacting the nucleic acid with one or more specific cleavage agents. The term “specific cleavage agent” as used herein refers to an agent, sometimes a chemical or an enzyme that can cleave a nucleic acid at one or more specific sites. Specific cleavage agents often cleave specifically according to a particular nucleotide sequence at a particular site.

Examples of enzymatic specific cleavage agents include without limitation endonucleases (e.g., DNase (e.g., DNase I, II); RNase (e.g., RNase E, F, H, P); Cleavase™ enzyme; Taq DNA polymerase; E. coli DNA polymerase I and eukaryotic structure-specific endonucleases; murine FEN-1 endonucleases; type I, II or III restriction endonucleases such as Acc I, Afl III, Alu I, Alw44 I, Apa I, Asn I, Ava I, Ava II, BamH I, Ban II, Bcl I, Bgl I. Bgl II, Bln I, Bsm I, BssH II, BstE II, Cfo I, Cla I, Dde I, Dpn I, Dra I, EcIX I, EcoR I, EcoR I, EcoR II, EcoR V, Hae II, Hae II, Hind II, Hind Ill, Hpa I, Hpa II, Kpn I, Ksp I, Mlu I, MIuN I, Msp I, Nci I, Nco I, Nde I, Nde II, Nhe I, Not I, Nru I, Nsi I, Pst I, Pvu I, Pvu II, Rsa I, Sac I, Sal I, Sau3A I, Sca I, ScrF I, Sfi I, Sma I, Spe I, Sph I, Ssp I, Stu I, Sty I, Swa I, Taq I, Xba I, Xho I; glycosylases (e.g., uracil-DNA glycolsylase (UDG), 3-methyladenine DNA glycosylase, 3-methyladenine DNA glycosylase 1l, pyrimidine hydrate-DNA glycosylase, FaPy-DNA glycosylase, thymine mismatch-DNA glycosylase, hypoxanthine-DNA glycosylase, 5-Hydroxymethyluracil DNA glycosylase (HmUDG), 5-Hydroxymethylcytosine DNA glycosylase, or 1,N6-etheno-adenine DNA glycosylase); exonucleases (e.g., exonuclease Ill); ribozymes, and DNAzymes. Nucleic acid may be treated with a chemical agent, and the modified nucleic acid may be cleaved. In non-limiting examples, nucleic acid may be treated with (i) alkylating agents such as methylnitrosourea that generate several alkylated bases, including N3-methyladenine and N3-methylguanine, which are recognized and cleaved by alkyl purine DNA-glycosylase; (ii) sodium bisulfite, which causes deamination of cytosine residues in DNA to form uracil residues that can be cleaved by uracil N-glycosylase; and (iii) a chemical agent that converts guanine to its oxidized form, 8-hydroxyguanine, which can be cleaved by formamidopyrimidine DNA N-glycosylase. Examples of chemical cleavage processes include without limitation alkylation, (e.g., alkylation of phosphorothioate-modified nucleic acid); cleavage of acid lability of P3′-N5′-phosphoroamidate-containing nucleic acid; and osmium tetroxide and piperidine treatment of nucleic acid.

Nucleic acid also may be exposed to a process that modifies certain nucleotides in the nucleic acid before providing nucleic acid for a method described herein. A process that selectively modifies nucleic acid based upon the methylation state of nucleotides therein can be applied to nucleic acid, for example. In addition, conditions such as high temperature, ultraviolet radiation, x-radiation, can induce changes in the sequence of a nucleic acid molecule. Nucleic acid may be provided in any form useful for conducting a sequence analysis or manufacture process described herein, such as solid or liquid form, for example. In certain embodiments, nucleic acid may be provided in a liquid form optionally comprising one or more other components, including without limitation one or more buffers or salts.

Nucleic acid may be single or double stranded. Single stranded DNA, for example, can be generated by denaturing double stranded DNA by heating or by treatment with alkali, for example. In some cases, nucleic acid is in a D-loop structure, formed by strand invasion of a duplex DNA molecule by an oligonucleotide or a DNA-like molecule such as peptide nucleic acid (PNA). D loop formation can be facilitated by addition of E. Coli RecA protein and/or by alteration of salt concentration, for example, using methods known in the art.

### Genomic DNA Target Sequences

In some embodiments of the methods provided herein, one or more nucleic acid species, and sometimes one or more nucleotide sequence species, are targeted for amplification and quantification. In some embodiments, the targeted nucleic acids are genomic DNA sequences. Certain genomic DNA target sequences are used, for example, because they can allow for the determination of a particular feature for a given assay. Genomic DNA target sequences can be referred to herein as markers for a given assay. In some cases, genomic target sequences are polymorphic, as described herein. In some embodiments, more than one genomic DNA target sequence or marker can allow for the determination of a particular feature for a given assay. Such genomic DNA target sequences are considered to be of a particular “region”. As used herein, a “region” is not intended to be limited to a description of a genomic location, such as a particular chromosome, stretch of chromosomal DNA or genetic locus. Rather, the term “region” is used herein to identify a collection of one or more genomic DNA target sequences or markers that can be indicative of a particular assay. Such assays can include, but are not limited to, assays for the detection and quantification of fetal nucleic acid, assays for the detection and quantification of maternal nucleic acid, assays for the detection and quantification of total DNA, assays for the detection and quantification of methylated DNA, assays for the detection and quantification of fetal specific nucleic acid (e.g. chromosome Y DNA), and assays for the detection and quantification of digested and/or undigested DNA, as an indicator of digestion efficiency. In some embodiments, the genomic DNA target sequence is described as being within a particular genomic locus. As used herein, a genomic locus can include any or a combination of open reading frame DNA, non-transcribed DNA, intronic sequences, extronic sequences, promoter sequences, enhancer sequences, flanking sequences, or any sequences considered by one of skill in the art to be associated with a given genomic locus.

### Assays for the Determination of Methylated DNA

In some embodiments of the methods provided herein, one or more genomic DNA target sequences are used that can allow for the determination of methylated DNA. Generally, genomic DNA target sequences used for the determination of methylated DNA are differentially methylated in fetal and maternal nucleic acid, and thus, differentially digested according to the methods provided herein for methylation-sensitive restriction enzymes. In some cases, a genomic DNA target sequence is a single copy gene. In some cases, a genomic DNA target sequence is located on chromosome 13, chromosome 18, chromosome 21, chromosome X, or chromosome Y. In some cases, a genomic DNA target sequence is not located on chromosome 13. In some cases, a genomic DNA target sequence is not located on chromosome 18. In some cases, a genomic DNA target sequence is not located on chromosome 21. In some cases, a genomic DNA target sequence is not located on chromosome X. In some cases, a genomic DNA target sequence is not located on chromosome Y. In some cases, a genomic DNA target sequence is typically methylated in one DNA species such as, for example, placental DNA (i.e. at least about 50% or greater methylation). In some cases, the genomic DNA target sequence is minimally methylated in another DNA species such as, for example, maternal DNA (i.e. less than about 1% methylation). In some cases, the genomic DNA target sequence does not contain any known single nucleotide polymorphisms (SNPs) within the PCR primer hybridization sequences. In some cases, the genomic DNA target sequence does not contain any known mutations within the PCR primer hybridization sequences. In some cases, the genomic DNA target sequence does not contain any known insertion or deletions within the PCR primer hybridization sequences. In some cases, the melting temperature of the PCR primers that can hybridize to a genomic DNA target sequence is not below 65° C. In some cases, the melting temperature of the PCR primers that can hybridize to a genomic DNA target sequence is not above 75° C. In some cases, the genomic DNA target sequence contains at least two restriction sites within the amplified region. In some embodiments, the genomic DNA target sequence length is about 50 base pairs to about 200 base pairs. In some cases, the genomic DNA target sequence length is 70 base pairs. In some cases, the genomic DNA target sequence does not possess any negative AG values for secondary structure of the complete amplicon prediction using mfold (M. Zuker, Mfold web server for nucleic acid folding and hybridization prediction. Nucleic Acids Res. 31 (13), 3406-15, (2003)). In some embodiments, the genomic DNA target sequence used for the determination of methylated DNA is within the TBX3 locus. In some embodiments, the genomic DNA target sequence used for the determination of methylated DNA is within the SOX14 locus. Additional genomic targets that can be used for the determination of methylated DNA in conjunction with the methods provided herein are presented in Example 3.

### Assays for the Determination of Total DNA

In some embodiments of the methods provided herein, one or more genomic DNA target sequences are used that can allow for the determination of total DNA. Generally, genomic DNA target sequences used for the determination of total DNA are present in every genome copy (e.g. is present in fetal DNA and maternal DNA, cancer DNA and normal DNA, pathogen DNA and host DNA). In some cases, a genomic DNA target sequence is a single copy gene. In some cases, a genomic DNA target sequence is located on chromosome 13, chromosome 18, chromosome 21, chromosome X, or chromosome Y. In some cases, a genomic DNA target sequence is not located on chromosome 13. In some cases, a genomic DNA target sequence is not located on chromosome 18. In some cases, a genomic DNA target sequence is not located on chromosome 21. In some cases, a genomic DNA target sequence is not located on chromosome X. In some cases, a genomic DNA target sequence is not located on chromosome Y. In some cases, a genomic DNA target sequence does not contain any known single nucleotide polymorphisms (SNPs) within the PCR primer hybridization sequences. In some cases, a genomic DNA target sequence does not contain any known mutations within the PCR primer hybridization sequences. In some cases, a genomic DNA target sequence does not contain any known insertion or deletions within the PCR primer hybridization sequences. In some cases, the melting temperature of the PCR primers that can hybridize to a genomic DNA target sequence is not below 65° C. In some cases, the melting temperature of the PCR primers that can hybridize to a genomic DNA target sequence is not above 75° C. In some embodiments, the genomic DNA target sequence length is about 50 base pairs to about 200 base pairs. In some cases, the genomic DNA target sequence length is 70 base pairs. In some cases, the genomic DNA target sequence does not possess any negative AG values for secondary structure of the complete amplicon prediction using mfold (M. Zuker, Mfold web server for nucleic acid folding and hybridization prediction. Nucleic Acids Res. 31 (13), 3406-15, (2003)). In some embodiments, the genomic DNA target sequence used for the determination of total DNA is within the ALB locus. In some embodiments, the genomic DNA target sequence used for the determination of total DNA is within the APOE or RNAseP locus.

### Assays for the Determination of Fetal DNA

In some embodiments of the methods provided herein, one or more genomic DNA target sequences are used that can allow for the determination of fetal DNA. In some embodiments, genomic DNA target sequences used for the determination of fetal DNA are specific to the Y chromosome. In some cases, the genomic DNA target sequence is a single copy gene. In some cases, the genomic DNA target sequence does not contain any known single nucleotide polymorphisms (SNPs) within the PCR primer hybridization sequences. In some cases, the genomic DNA target sequence does not contain any known mutations within the PCR primer hybridization sequences. In some cases, the genomic DNA target sequence does not contain any known insertion or deletions within the PCR primer hybridization sequences. In some cases, the melting temperature of the PCR primers that can hybridize to a genomic DNA target sequence is not below 65° C. In some cases, the melting temperature of the PCR primers that can hybridize to a genomic DNA target sequence is not above 75° C. In some cases, the genomic DNA target sequence does not contain the restriction site GCGC within the amplified region. In some embodiments, the genomic DNA target sequence length is about 50 base pairs to about 200 base pairs. In some cases, the genomic DNA target sequence length is 70 base pairs. In some cases, the genomic DNA target sequence does not possess any negative AG values for secondary structure of the complete amplicon prediction using mfold (M. Zuker, Mfold web server for nucleic acid folding and hybridization prediction. Nucleic Acids Res. 31 (13), 3406-15, (2003)). In some embodiments, the genomic DNA target sequence used for the determination of fetal DNA is within the UTY locus. In some embodiments, the genomic DNA target sequence used for the determination of fetal DNA is within the SRY1 or SRY2 locus.

Assays for the Determination of Digested and/or Undigested DNA

In some embodiments of the methods provided herein, one or more genomic DNA target sequences are used that can allow for the determination of the amount of digested or undigested nucleic acid, as an indicator of digestion efficiency. Such genomic DNA target sequences are present in every genome in the sample (e.g. maternal and fetal species genomes). Generally, genomic DNA target sequences used for the determination of digested or undigested DNA contain at least one restriction site present in a genomic DNA target sequence used in another assay. Thus, the genomic DNA target sequences used for the determination of digested or undigested DNA serve as controls for assays that include differential digestion. Generally, the genomic DNA target sequence is unmethylated in all nucleic acid species tested (e.g. unmethylated in both maternal and fetal species genomes). In some cases, the genomic DNA target sequence is a single copy gene. In some cases, the genomic DNA target sequence is not located on chromosome 13. In some cases, the genomic DNA target sequence is not located on chromosome 18. In some cases, the genomic DNA target sequence is not located on chromosome 21. In some cases, the genomic DNA target sequence is not located on chromosome X. In some cases, the genomic DNA target sequence is not located on chromosome Y. In some cases, the genomic DNA target sequence does not contain any known single nucleotide polymorphisms (SNPs) within the PCR primer hybridization sequences. In some cases, the genomic DNA target sequence does not contain any known mutations within the PCR primer hybridization sequences. In some cases, the genomic DNA target sequence does not contain any known insertion or deletions within the PCR primer hybridization sequences. In some cases, the melting temperature of the PCR primers that can hybridize to a genomic DNA target sequence is not below 65° C. In some cases, the melting temperature of the PCR primers that can hybridize to a genomic DNA target sequence is not above 75° C. In some embodiments, the genomic DNA target sequence length is about 50 base pairs to about 200 base pairs. In some cases, the genomic DNA target sequence length is 70 base pairs. In some cases, the genomic DNA target sequence does not possess any negative AG values for secondary structure of the complete amplicon prediction using mfold (M. Zuker, Mfold web server for nucleic acid folding and hybridization prediction. Nucleic Acids Res. 31 (13), 3406-15, (2003)). In some embodiments, the genomic DNA target sequence used for the determination of digested or undigested DNA is within the POP5 locus. In some embodiments, the genomic DNA target sequence used for the determination of digested or undigested DNA is within the LDHA locus.

### Methylation Specific Separation of Nucleic Acid

The methods provided herein offer an alternative approach for the enrichment of fetal DNA based on the methylation-specific separation of differentially methylated DNA. It has recently been discovered that many genes involved in developmental regulation are controlled through epigenetics in embryonic stem cells. Consequently, multiple genes can be expected to show differential DNA methylation between nucleic acid of fetal origin and maternal origin. Once these regions are identified, a technique to capture methylated DNA can be used to specifically enrich fetal DNA. For identification of differentially methylated regions, a novel approach was used to capture methylated DNA. This approach uses a protein, in which the methyl binding domain of MBD2 is fused to the Fc fragment of an antibody (MBD-FC) (Gebhard C, Schwarzfischer L, Pham T H, Schilling E, Klug M, Andreesen R, Rehli M (2006) Genome wide profiling of CpG methylation identifies novel targets of aberrant hypermethylation in myeloid leukemia. Cancer Res 66:6118-6128). This fusion protein has several advantages over conventional methylation specific antibodies. The MBD-FC has a higher affinity to methylated DNA and it binds double stranded DNA. Most importantly the two proteins differ in the way they bind DNA. Methylation specific antibodies bind DNA stochastically, which means that only a binary answer can be obtained. The methyl binding domain of MBD-FC on the other hand binds all DNA molecules regardless of their methylation status. The strength of this protein—DNA interaction is defined by the level of DNA methylation. After binding genomic DNA, eluate solutions of increasing salt concentrations can be used to fractionate non-methylated and methylated DNA allowing for a more controlled separation (Gebhard C, Schwarzfischer L, Pham T H, Andreesen R, Mackensen A, Rehli M (2006) Rapid and sensitive detection of CpG-methylation using methyl-binding (MB)-PCR. Nucleic Acids Res 34:e82). Consequently this method, called Methyl-CpG immunoprecipitation (MCIP), cannot only enrich, but also fractionate genomic DNA according to methylation level, which is particularly helpful when the unmethylated DNA fraction should be investigated as well.

### Methylation Sensitive Restriction Enzyme Digestion

The technology herein also provides compositions and processes for determining the amount of fetal nucleic acid from a maternal sample. The technology herein allows for the enrichment of fetal nucleic acid regions in a maternal sample by selectively digesting nucleic acid from said maternal sample with an enzyme that selectively and completely or substantially digests the maternal nucleic acid to enrich the sample for at least one fetal nucleic acid region. Preferably, the digestion efficiency is greater than about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. Following enrichment, the amount of fetal nucleic acid can be determined by quantitative methods that do not require polymorphic sequences or bisulfite treatment, thereby, offering a solution that works equally well for female fetuses and across different ethnicities and preserves the low copy number fetal nucleic acid present in the sample.

For example, there are methyl-sensitive enzymes that preferentially or substantially cleave or digest at their DNA recognition sequence if it is non-methylated. Thus, an unmethylated DNA sample will be cut into smaller fragments than a methylated DNA sample. Similarly, a hypermethylated DNA sample will not be cleaved. In contrast, there are methyl-sensitive enzymes that cleave at their DNA recognition sequence only if it is methylated.

Methyl-sensitive enzymes that digest unmethylated DNA suitable for use in methods of the technology herein include, but are not limited to, HpaII, HhaI, MaeII, BstUI and AciI. An enzyme that can be used is HpaII that cuts only the unmethylated sequence CCGG. Another enzyme that can be used is HhaI that cuts only the unmethylated sequence GCGC. Both enzymes are available from New England BioLabs®, Inc. Combinations of two or more methyl-sensitive enzymes that digest only unmethylated DNA can also be used. Suitable enzymes that digest only methylated DNA include, but are not limited to, Dpnl, which cuts at a recognition sequence GATC, and McrBC, which belongs to the family of AAA⁺ proteins and cuts DNA containing modified cytosines and cuts at recognition site 5′ . . . Pu^{m}C (N₄₀₋₃₀₀₀) Pu^{m}C . . . 3′ (New England BioLabs, Inc., Beverly, Mass.).

Cleavage methods and procedures for selected restriction enzymes for cutting DNA at specific sites are well known to the skilled artisan. For example, many suppliers of restriction enzymes provide information on conditions and types of DNA sequences cut by specific restriction enzymes, including New England BioLabs, Pro-Mega Biochems, Boehringer-Mannheim, and the like. Sambrook et al. (See Sambrook et al., Molecular Biology: A laboratory Approach, Cold Spring Harbor, N.Y. 1989) provide a general description of methods for using restriction enzymes and other enzymes. Enzymes often are used under conditions that will enable cleavage of the maternal DNA with about 95%-100% efficiency, preferably with about 98%-100% efficiency.

### Other Methods for Methylation Analysis

Various methylation analysis procedures are known in the art, and can be used in conjunction with the present technology. These assays allow for determination of the methylation state of one or a plurality of CpG islands within a DNA sequence. In addition, the methods maybe used to quantify methylated nucleic acid. Such assays involve, among other techniques, DNA sequencing of bisulfite-treated DNA, PCR (for sequence-specific amplification), Southern blot analysis, and use of methylation-sensitive restriction enzymes.

Genomic sequencing is a technique that has been simplified for analysis of DNA methylation patterns and 5-methylcytosine distribution by using bisulfite treatment (Frommer et al., Proc. Natl. Acad. Sci. USA 89:1827-1831, 1992). Additionally, restriction enzyme digestion of PCR products amplified from bisulfite-converted DNA may be used, e.g., the method described by Sadri & Hornsby (Nucl. Acids Res. 24:5058-5059, 1996), or COBRA (Combined Bisulfite Restriction Analysis) (Xiong & Laird, Nucleic Acids Res. 25:2532-2534, 1997).

COBRA analysis is a quantitative methylation assay useful for determining DNA methylation levels at specific gene loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the interested CpG islands, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples. Typical reagents (e.g., as might be found in a typical COBRA-based kit) for COBRA analysis may include, but are not limited to: PCR primers for specific gene (or methylation-altered DNA sequence or CpG island); restriction enzyme and appropriate buffer; gene-hybridization oligo; control hybridization oligo; kinase labeling kit for oligo probe; and radioactive nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components.

The MethyLight™ assay is a high-throughput quantitative methylation assay that utilizes fluorescence-based real-time PCR (TaqMan®) technology that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed either in an “unbiased” (with primers that do not overlap known CpG methylation sites) PCR reaction, or in a “biased” (with PCR primers that overlap known CpG dinucleotides) reaction. Sequence discrimination can occur either at the level of the amplification process or at the level of the fluorescence detection process, or both.

The MethyLight assay may be used as a quantitative test for methylation patterns in the genomic DNA sample, where sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe overlie any CpG dinucleotides. Alternatively, a qualitative test for genomic methylation is achieved by probing of the biased PCR pool with either control oligonucleotides that do not “cover” known methylation sites (a fluorescence-based version of the “MSP” technique), or with oligonucleotides covering potential methylation sites.

The MethyLight process can by used with a “TaqMan” probe in the amplification process. For example, double-stranded genomic DNA is treated with sodium bisulfite and subjected to one of two sets of PCR reactions using TaqMan® probes; e.g., with either biased primers and TaqMan® probe, or unbiased primers and TaqMan® probe. The TaqMan® probe is dual-labeled with fluorescent “reporter” and “quencher” molecules, and is designed to be specific for a relatively high GC content region so that it melts out at about 10.degree. C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5 to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight™-based kit) for MethyLight™ analysis may include, but are not limited to: PCR primers for specific gene (or methylation-altered DNA sequence or CpG island); TaqMan® probes; optimized PCR buffers and deoxynucleotides; and Taq polymerase.

The Ms-SNuPE technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest. Small amounts of DNA can be analyzed (e.g., microdissected pathology sections), and it avoids utilization of restriction enzymes for determining the methylation status at CpG sites.

Typical reagents (e.g., as might be found in a typical Ms-SNuPE-based kit) for Ms-SNuPE analysis may include, but are not limited to: PCR primers for specific gene (or methylation-altered DNA sequence or CpG island); optimized PCR buffers and deoxynucleotides; gel extraction kit; positive control primers; Ms-SNuPE primers for specific gene; reaction buffer (for the Ms-SNuPE reaction); and radioactive nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

MSP (methylation-specific PCR) allows for assessing the methylation status of virtually any group of CpG sites within a CpG island, independent of the use of methylation-sensitive restriction enzymes (Herman et al. Proc. Nat. Acad. Sci. USA 93:9821-9826, 1996; U.S. Pat. No. 5,786,146). Briefly, DNA is modified by sodium bisulfite converting all unmethylated, but not methylated cytosines to uracil, and subsequently amplified with primers specific for methylated versus umnethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylated and unmethylated PCR primers for specific gene (or methylation-altered DNA sequence or CpG island), optimized PCR buffers and deoxynucleotides, and specific probes.

The MCA technique is a method that can be used to screen for altered methylation patterns in genomic DNA, and to isolate specific sequences associated with these changes (Toyota et al., Cancer Res. 59:2307-12, 1999). Briefly, restriction enzymes with different sensitivities to cytosine methylation in their recognition sites are used to digest genomic DNAs from primary tumors, cell lines, and normal tissues prior to arbitrarily primed PCR amplification. Fragments that show differential methylation are cloned and sequenced after resolving the PCR products on high-resolution polyacrylamide gels. The cloned fragments are then used as probes for Southern analysis to confirm differential methylation of these regions. Typical reagents (e.g., as might be found in a typical MCA-based kit) for MCA analysis may include, but are not limited to: PCR primers for arbitrary priming Genomic DNA; PCR buffers and nucleotides, restriction enzymes and appropriate buffers; gene-hybridization oligos or probes; control hybridization oligos or probes.

Another method for analyzing methylation sites is a primer extension assay, including an optimized PCR amplification reaction that produces amplified targets for subsequent primer extension genotyping analysis using mass spectrometry. The assay can also be done in multiplex. This method (particularly as it relates to genotyping single nucleotide polymorphisms) is described in detail in PCT publication WO05012578A1 and US publication US20050079521A1. For methylation analysis, the assay can be adopted to detect bisulfite introduced methylation dependent C to T sequence changes. These methods are particularly useful for performing multiplexed amplification reactions and multiplexed primer extension reactions (e.g., multiplexed homogeneous primer mass extension (hME) assays) in a single well to further increase the throughput and reduce the cost per reaction for primer extension reactions.

Four additional methods for DNA methylation analysis include restriction landmark genomic scanning (RLGS, Costello et al., 2000), methylation-sensitive-representational difference analysis (MS-RDA), methylation-specific AP-PCR (MS-AP-PCR) and methyl-CpG binding domain column/segregation of partly melted molecules (MBD/SPM).

Additional methylation analysis methods that may be used in conjunction with the present technology are described in the following papers: Laird, P. W. Nature Reviews Cancer 3, 253-266 (2003); Biotechniques; Uhlmann, K. et al. Electrophoresis 23:4072-4079 (2002)—PyroMeth; Colella et al. Biotechniques. 2003 July; 35(1):146-50; Dupont J M, Tost J, Jammes H, and Gut I G. Anal Biochem, October 2004; 333(1): 119-27; and Tooke N and Pettersson M. IVDT. November 2004; 41.

### Nucleic Acid Quantification

In some embodiments, the amount of fetal nucleic acid in a sample is determined. In some cases, the amount of fetal nucleic acid is determined based on a quantification of sequence read counts described herein. Quantification may be achieved by direct counting of sequence reads covering particular methylation sites and/or target sites, or by competitive PCR (i.e., co-amplification of competitor oligonucleotides of known quantity, as described herein). The term “amount” as used herein with respect to nucleic acids refers to any suitable measurement, including, but not limited to, absolute amount (e.g. copy number), relative amount (e.g. fraction or ratio), weight (e.g., grams), and concentration (e.g., grams per unit volume (e.g., milliliter); molar units).

### Fraction Determination

In some embodiments, a fraction or ratio can be determined for the amount of one nucleic acid relative to the amount of another nucleic acid. In some embodiments, the fraction of fetal nucleic acid in a sample relative to the total amount of nucleic acid in the sample is determined. To calculate the fraction of fetal nucleic acid in a sample relative to the total amount of the nucleic acid in the sample, the following equation can be applied:

The fraction of fetal nucleic acid=(amount of fetal nucleic acid)/[(amount of total nucleic acid)].

### Copy Number Determination Using Competitors

In some embodiments, the absolute amount (e.g. copy number) of fetal nucleic acid is determined. Often, the copy number of fetal nucleic acid is determined based on the amount of a competitor oligonucleotide used. In some embodiments, the copy number of maternal nucleic acid is determined. To calculate the copy number of fetal nucleic acid in a sample, the following equation can be applied:

Copy number(fetal nucleic acid)=[(amount of the fetal nucleic acid)/(amount of the fetal competitor)]×C

where C is the number of competitor oligonucleotides added into the reaction. In some cases, the amounts of the fetal nucleic acid and fetal competitor are obtained in a readout generated by a sequencing reaction (e.g. sequence read counts).

### Additional Methods for Determining Fetal Nucleic Acid Content

The amount of fetal nucleic acid (e.g., concentration, relative amount, absolute amount, copy number, and the like) in nucleic acid is determined in some embodiments. In some cases, the amount of fetal nucleic acid in a sample is referred to as “fetal fraction”. In certain embodiments, the amount of fetal nucleic acid is determined according to markers specific to a male fetus (e.g., Y-chromosome STR markers (e.g., DYS 19, DYS 385, DYS 392 markers); RhD marker in RhD-negative females), allelic ratios of polymorphic sequences, or according to one or more markers specific to fetal nucleic acid and not maternal nucleic acid (e.g., differential epigenetic biomarkers (e.g., methylation; described in further detail below) between mother and fetus, or fetal RNA markers in maternal blood plasma (see e.g., Lo, 2005, Journal of Histochemistry and Cytochemistry 53 (3): 293-296)).

### Polymorphism-Based Fetal Quantifier Assay

Determination of fetal nucleic acid content (e.g., fetal fraction) sometimes is performed using a polymorphism-based fetal quantifier assay (FQA), as described herein. This type of assay allows for the detection and quantification of fetal nucleic acid in a maternal sample based on allelic ratios of polymorphic sequences (e.g., single nucleotide polymorphisms (SNPs)). In some cases, nucleotide sequence reads are obtained for a maternal sample and fetal fraction is determined by comparing the total number of nucleotide sequence reads that map to a first allele and the total number of nucleotide sequence reads that map to a second allele at an informative polymorphic site (e.g., SNP) in a reference genome. In some cases, fetal alleles are identified, for example, by their relative minor contribution to the mixture of fetal and maternal nucleic acids in the sample when compared to the major contribution to the mixture by the maternal nucleic acids. In some cases, fetal alleles are identified by a deviation of allele frequency from an expected allele frequency, as described below. In some cases, the relative abundance of fetal nucleic acid in a maternal sample can be determined as a parameter of the total number of unique sequence reads mapped to a target nucleic acid sequence on a reference genome for each of the two alleles of a polymorphic site. In some cases, the relative abundance of fetal nucleic acid in a maternal sample can be determined as a parameter of the relative number of sequence reads for each allele from an enriched sample.

In some embodiments, determining fetal fraction comprises enriching a sample nucleic acid for one or more polymorphic nucleic acid targets. In some cases, a plurality of polymorphic targets is enriched. A plurality of polymorphic nucleic acid targets is sometimes referred to as a collection or a panel (e.g., target panel, SNP panel, SNP collection). A plurality of polymorphic targets can comprise two or more targets. For example, a plurality of polymorphic targets can comprise 2, 3, 4, 5, 6, 7, 8, 9, 10, 20, 30, 40, 50, 60, 70, 80, 90, 100, 200, 300, 400, 500, 600, 700, 800, 900, 1000, or more targets. In some cases, 10 or more polymorphic nucleic acid targets are enriched. In some cases, 50 or more polymorphic nucleic acid targets are enriched. In some cases, 100 or more polymorphic nucleic acid targets are enriched. In some cases, 500 or more polymorphic nucleic acid targets are enriched. In some cases, about 10 to about 500 polymorphic nucleic acid targets are enriched. In some cases, about 20 to about 400 polymorphic nucleic acid targets are enriched. In some cases, about 30 to about 200 polymorphic nucleic acid targets are enriched. In some cases, about 40 to about 100 polymorphic nucleic acid targets are enriched. In some cases, about 60 to about 90 polymorphic nucleic acid targets are enriched. For example, in certain embodiments, about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 polymorphic nucleic acid targets are enriched.

In some embodiments, at least one polymorphic nucleic acid target of the plurality of polymorphic nucleic acid targets is informative for determining fetal fraction in a given sample. A polymorphic nucleic acid target that is informative for determining fetal fraction, sometimes referred to as an informative target, informative polymorphism, or informative SNP, typically differs in some aspect between the fetus and the mother. For example, an informative target may have one allele for the fetus and a different allele for the mother (e.g., the mother has allele A at the polymorphic target and the fetus has allele B at the polymorphic target site). Typically, a fetal allele that differs from either of the maternal alleles is paternally inherited (i.e., is from the father). Thus, paternally inherited alleles that differ from maternal alleles can be useful for identifying and/or quantifying fetal nucleic acid (e.g., determining fetal fraction).

In some cases, polymorphic nucleic acid targets are informative in the context of certain maternal/fetal genotype combinations. For a biallelic polymorphic target (i.e., two possible alleles (e.g., A and B)), possible maternal/fetal genotype combinations include: 1) maternal AA, fetal AA; 2) maternal AA, fetal AB; 3) maternal AB, fetal AA; 4) maternal AB, fetal AB; 5) maternal AB; fetal BB; 6) maternal BB, fetal AB; and 7) maternal BB, fetal BB. Genotypes AA and BB are considered homozygous genotypes and genotype AB is considered a heterozygous genotype. In some cases, informative genotype combinations (i.e., genotype combinations for a polymorphic nucleic acid target that may be informative for determining fetal fraction) include combinations where the mother is homozygous and the fetus is heterozygous (e.g., maternal AA, fetal AB; or maternal BB, fetal AB). Such genotype combinations may be referred to as Type 1 informative genotypes or informative heterozygotes. In some cases, informative genotype combinations (i.e., genotype combinations for a polymorphic nucleic acid target that may be informative for determining fetal fraction) include combinations where the mother is heterozygous and the fetus is homozygous (e.g., maternal AB, fetal AA; or maternal AB, fetal BB). Such genotype combinations may be referred to as Type 2 informative genotypes or informative homozygotes. In some cases, non-informative genotype combinations (i.e., genotype combinations for a polymorphic nucleic acid target that may not be informative for determining fetal fraction) include combinations where the mother is heterozygous and the fetus is heterozygous (e.g., maternal AB, fetal AB). Such genotype combinations may be referred to as non-informative genotypes or non-informative heterozygotes. In some cases, non-informative genotype combinations (i.e., genotype combinations for a polymorphic nucleic acid target that may not be informative for determining fetal fraction) include combinations where the mother is homozygous and the fetus is homozygous (e.g., maternal AA, fetal AA; or maternal BB, fetal BB). Such genotype combinations may be referred to as non-informative genotypes or non-informative homozygotes.

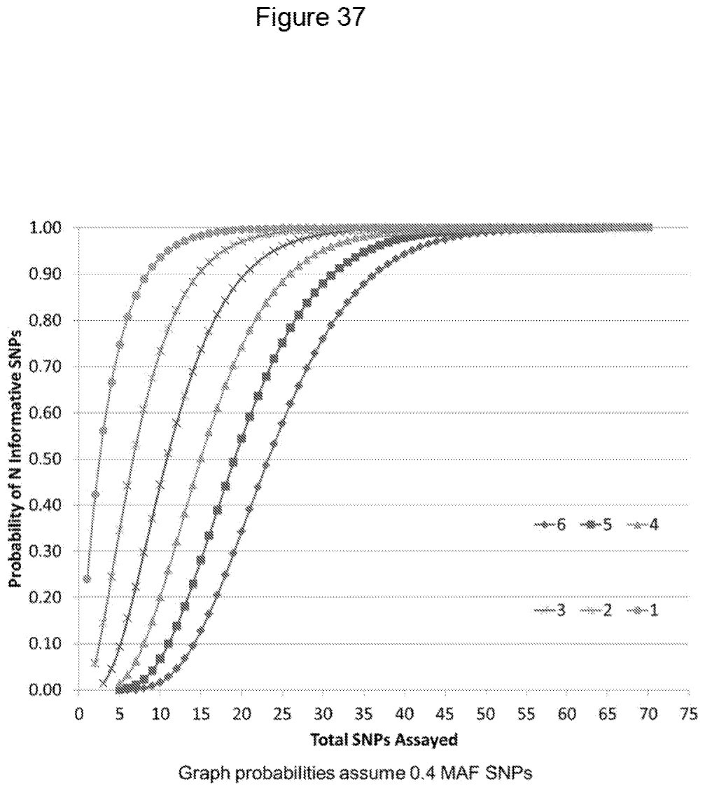

In some embodiments, individual polymorphic nucleic acid targets and/or panels of polymorphic nucleic acid targets are selected based on certain criteria, such as, for example, minor allele population frequency, variance, coefficient of variance, MAD value, and the like. In some cases, polymorphic nucleic acid targets are selected so that at least one polymorphic nucleic acid target within a panel of polymorphic targets has a high probability of being informative for a majority of samples tested. Additionally, in some cases, the number of polymorphic nucleic acid targets (i.e., number of targets in a panel) is selected so that least one polymorphic nucleic acid target has a high probability of being informative for a majority of samples tested. For example, selection of a larger number of polymorphic targets generally increases the probability that least one polymorphic nucleic acid target will be informative for a majority of samples tested (see, FIG. 37, for example).

In some cases, the polymorphic nucleic acid targets and number thereof (e.g., number of polymorphic targets selected for enrichment) result in at least about 2 to about 50 or more polymorphic nucleic acid targets being informative for determining the fetal fraction for at least about 80% to about 100% of samples. For example, the polymorphic nucleic acid targets and number thereof result in at least about 5, 10, 15, 20, 25, 30, 35, 40, 45, 50 or more polymorphic nucleic acid targets being informative for determining the fetal fraction for at least about 81%, 82%, 83%, 84%, 85%, 86%, 87%, 88%, 89%, 90%, 91%, 92%, 93%, 94%, 95%, 96%, 97%, 98%, or 99% of samples. In some cases, the polymorphic nucleic acid targets and number thereof result in at least five polymorphic nucleic acid targets being informative for determining the fetal fraction for at least 90% of samples. In some cases, the polymorphic nucleic acid targets and number thereof result in at least five polymorphic nucleic acid targets being informative for determining the fetal fraction for at least 95% of samples. In some cases, the polymorphic nucleic acid targets and number thereof result in at least five polymorphic nucleic acid targets being informative for determining the fetal fraction for at least 99% of samples. In some cases, the polymorphic nucleic acid targets and number thereof result in at least ten polymorphic nucleic acid targets being informative for determining the fetal fraction for at least 90% of samples. In some cases, the polymorphic nucleic acid targets and number thereof result in at least ten polymorphic nucleic acid targets being informative for determining the fetal fraction for at least 95% of samples. In some cases, the polymorphic nucleic acid targets and number thereof result in at least ten polymorphic nucleic acid targets being informative for determining the fetal fraction for at least 99% of samples.

In some embodiments, individual polymorphic nucleic acid targets are selected based, in part, on minor allele population frequency. In some cases, polymorphic nucleic acid targets having minor allele population frequencies of about 10% to about 50% are selected. For example, polymorphic nucleic acid targets having minor allele population frequencies of about 15%, 20%, 25%, 30%, 35%, 36%, 37%, 38%, 39%, 40%, 41%, 42%, 43%, 44%, 45%, 46%, 47%, 48%, or 49% are selected. In some embodiments, polymorphic nucleic acid targets having a minor allele population frequency of about 40% or more are selected.

In some embodiments, individual polymorphic nucleic acid targets and/or panels of polymorphic nucleic acid targets are selected based, in part, on degree of variance for an individual polymorphic target or a panel of polymorphic targets. Variance, in come cases, can be specific for certain polymorphic targets or panels of polymorphic targets and can be from systematic, experimental, procedural, and or inherent errors or biases (e.g., sampling errors, sequencing errors, PCR bias, and the like). Variance of an individual polymorphic target or a panel of polymorphic targets can be determined by any method known in the art for assessing variance and may be expressed, for example, in terms of a calculated variance, an error, standard deviation, p-value, mean absolute deviation, median absolute deviation, median adjusted deviation (MAD score), coefficient of variance (CV), and the like. In some embodiments, measured allele frequency variance (i.e., background allele frequency) for certain SNPs (when homozygous, for example) can be from about 0.001 to about 0.01 (i.e., 0.1% to about 1.0%). For example, measured allele frequency variance can be about 0.002, 0.003, 0.004, 0.005, 0.006, 0.007, 0.008, or 0.009. In some cases, measured allele frequency variance is about 0.007.

In some cases, noisy polymorphic targets are excluded from a panel of polymorphic nucleic acid targets selected for determining fetal fraction. The term “noisy polymorphic targets” or “noisy SNPs” refers to (a) targets or SNPs that have significant variance between data points (e.g., measured fetal fraction, measured allele frequency) when analyzed or plotted, (b) targets or SNPs that have significant standard deviation (e.g., greater than 1, 2, or 3 standard deviations), (c) targets or SNPs that have a significant standard error of the mean, the like, and combinations of the foregoing. Noise for certain polymorphic targets or SNPs sometimes occurs due to the quantity and/or quality of starting material (e.g., nucleic acid sample), sometimes occurs as part of processes for preparing or replicating DNA used to generate sequence reads, and sometimes occurs as part of a sequencing process. In certain embodiments, noise for some polymorphic targets or SNPs results from certain sequences being over represented when prepared using PCR-based methods. In some cases, noise for some polymorphic targets or SNPs results from one or more inherent characteristics of the site such as, for example, certain nucleotide sequences and/or base compositions surrounding, or being adjacent to, a polymorphic target or SNP. A SNP having a measured allele frequency variance (when homozygous, for example) of about 0.005 or more may be considered noisy. For example, a SNP having a measured allele frequency variance of about 0.006, 0.007, 0.008, 0.009, 0.01 or more may be considered noisy.

In some embodiments, variance of an individual polymorphic target or a panel of polymorphic targets can be represented using coefficient of variance (CV). Coefficient of variance (i.e., standard deviation divided by the mean) can be determined, for example, by determining fetal fraction for several aliquots of a single maternal sample comprising maternal and fetal nucleic acid, and calculating the mean fetal fraction and standard deviation. In some cases, individual polymorphic nucleic acid targets and/or panels of polymorphic nucleic acid targets are selected so that fetal fraction is determined with a coefficient of variance (CV) of 0.30 or less. For example, fetal fraction may determined with a coefficient of variance (CV) of 0.25, 0.20, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01 or less, in some embodiments. In some cases, fetal fraction is determined with a coefficient of variance (CV) of 0.20 or less. In some cases, fetal fraction is determined with a coefficient of variance (CV) of 0.10 or less. In some cases, fetal fraction is determined with a coefficient of variance (CV) of 0.05 or less.

In some embodiments, an allele frequency is determined for each of the polymorphic nucleic acid targets in a sample. This sometimes is referred to as measured allele frequency. Allele frequency can be determined, for example, by counting the number of sequence reads for an allele (e.g., allele B) and dividing by the total number of sequence reads for that locus (e.g., allele B+allele A). In some cases, an allele frequency average, mean or median is determined. Fetal fraction can be determined based on the allele frequency mean (e.g., allele frequency mean multiplied by two), in some cases.

In some embodiments, determining whether a polymorphic nucleic acid target is informative comprises comparing its measured allele frequency to a fixed cutoff frequency. In some cases, determining which polymorphic nucleic acid targets are informative comprises identifying informative genotypes by comparing each allele frequency to one or more fixed cutoff frequencies. Fixed cutoff frequencies may be predetermined threshold values based on one or more qualifying data sets, for example. In some cases, the fixed cutoff for identifying informative genotypes from non-informative genotypes is expressed as a percent (%) shift in allele frequency from an expected allele frequency. Generally, expected allele frequencies for a given allele (e.g., allele A) are 0 (for a BB genotype), 0.5 (for an AB genotype) and 1.0 (for an AA genotype), or equivalent values on any numerical scale. A deviation from an expected allele frequency that is beyond one or more fixed cutoff frequencies may be considered informative. The degree of deviation generally is proportional to fetal fraction (i.e., large deviations from expected allele frequency may be observed in samples having high fetal fraction).

In some cases, the fixed cutoff for identifying informative genotypes from non-informative homozygotes is about a 0.5% or greater shift in allele frequency. For example, a fixed cutoff may be about a 0.6%, 0.7%, 0.8%, 0.9%, 1%, 1.5%, 2%, 3%, 4%, 5%, 10% or greater shift in allele frequency. In some cases, the fixed cutoff for identifying informative genotypes from non-informative homozygotes is about a 1% or greater shift in allele frequency. In some cases, the fixed cutoff for identifying informative genotypes from non-informative homozygotes is about a 2% or greater shift in allele frequency. In some embodiments, the fixed cutoff for identifying informative genotypes from non-informative heterozygotes is about a 10% or greater shift in allele frequency. For example, a fixed cutoff may be about a 10%, 15%, 20%, 21%, 22%, 23%, 24%, 25%, 26%, 27%, 28%, 29%, 30%, 35%, 40%, 45%, 50%, 55%, 60%, 70%, 80% or greater shift in allele frequency. In some cases, the fixed cutoff for identifying informative genotypes from non-informative heterozygotes is about a 25% or greater shift in allele frequency. In some cases, the fixed cutoff for identifying informative genotypes from non-informative heterozygotes is about a 50% or greater shift in allele frequency.

In some embodiments, determining whether a polymorphic nucleic acid target is informative comprises comparing its measured allele frequency to a target-specific cutoff value. In some embodiments, target-specific cutoff frequencies are determined for each polymorphic nucleic acid target. Typically, target-specific cutoff frequency is determined based on the allele frequency variance for the corresponding polymorphic nucleic acid target. In some embodiments, variance of individual polymorphic targets can be represented by a median absolute deviation (MAD), for example. In some cases, determining a MAD value for each polymorphic nucleic acid target can generate unique (i.e., target-specific) cutoff values. To determine median absolute deviation, measured allele frequency can be determined, for example, for multiple replicates (e.g., 5, 6, 7, 8, 9, 10, 15, 20 or more replicates) of a maternal only nucleic acid sample (e.g., buffy coat sample). Each polymorphic target in each replicate will typically have a slightly different measured allele frequency due to PCR and/or sequencing errors, for example. A median allele frequency value can be identified for each polymorphic target. A deviation from the median for the remaining replicates can be calculated (i.e., the difference between the observed allele frequency and the median allele frequency). The absolute value of the deviations (i.e., negative values become positive) is taken and the median value of the absolute deviations is calculated to provide a median absolute deviation (MAD) for each polymorphic nucleic acid target. A target-specific cutoff can be assigned, for example, as a multiple of the MAD (e.g., 1×MAD, 2×MAD, 3×MAD, 4×MAD or 5×MAD). Typically, polymorphic targets having less variance have a lower MAD and therefore a lower cutoff value than more variable targets.

In some embodiments, enriching comprises amplifying the plurality of polymorphic nucleic acid targets. In some cases, the enriching comprises generating amplification products in an amplification reaction. Amplification of polymorphic targets may be achieved by any method described herein or known in the art for amplifying nucleic acid (e.g., PCR). In some cases, the amplification reaction is performed in a single vessel (e.g., tube, container, well on a plate) which sometimes is referred to herein as multiplexed amplification.

In some embodiments, certain parental genotypes are known prior to the enriching of polymorphic nucleic acid targets. In some cases, the maternal genotype for one or more polymorphic targets is known prior to enriching. In some cases, the paternal genotype for one or more polymorphic targets is known prior to enriching. In some cases, the maternal genotype and the paternal genotype for one or more polymorphic targets are known prior to enriching. In some embodiments, certain parental genotypes are not known prior to the enriching of polymorphic nucleic acid targets. In some cases, the maternal genotype for one or more polymorphic targets is not known prior to enriching. In some cases, the paternal genotype for one or more polymorphic targets is not known prior to enriching. In some cases, the maternal genotype and the paternal genotype for one or more polymorphic targets are not known prior to enriching. In some embodiments, parental genotypes are not known for any of the polymorphic nucleic acid targets prior to enriching. In some cases, the maternal genotype for each of the polymorphic targets is not known prior to enriching. In some cases, the paternal genotype for each of the polymorphic targets is not known prior to enriching. In some cases, the maternal genotype and the paternal genotype for each of the polymorphic targets are not known prior to enriching.

In some embodiments, the polymorphic nucleic acid targets each comprise at least one single nucleotide polymorphism (SNP). In some embodiments, the SNPs are selected from: rs10413687, rs10949838, rs1115649, rs11207002, rs11632601, rs11971741, rs12660563, rs13155942, rs1444647, rs1572801, rs17773922, rs1797700, rs1921681, rs1958312, rs196008, rs2001778, rs2323659, rs2427099, rs243992, rs251344, rs254264, rs2827530, rs290387, rs321949, rs348971, rs390316, rs3944117, rs425002, rs432586, rs444016, rs4453265, rs447247, rs4745577, rs484312, rs499946, rs500090, rs500399, rs505349, rs505662, rs516084, rs517316, rs517914, rs522810, rs531423, rs537330, rs539344, rs551372, rs567681, rs585487, rs600933, rs619208, rs622994, rs639298, rs642449, rs6700732, rs677866, rs683922, rs686851, rs6941942, rs7045684, rs7176924, rs7525374, rs870429, rs949312, rs9563831, rs970022, rs985462, rs1005241, rs1006101, rs10745725, rs10776856, rs10790342, rsl 1076499, rs11103233, rs11133637, rs11974817, rs12102203, rs12261, rs12460763, rs12543040, rs12695642, rs13137088, rs13139573, rs1327501, rs13438255, rs1360258, rs1421062, rs1432515, rs1452396, rs1518040, rs16853186, rs1712497, rs1792205, rs1863452, rs1991899, rs2022958, rs2099875, rs2108825, rs2132237, rs2195979, rs2248173, rs2250246, rs2268697, rs2270893, rs244887, rs2736966, rs2851428, rs2906237, rs2929724, rs3742257, rs3764584, rs3814332, rs4131376, rs4363444, rs4461567, rs4467511, rs4559013, rs4714802, rs4775899, rs4817609, rs488446, rs4950877, rs530913, rs6020434, rs6442703, rs6487229, rs6537064, rs654065, rs6576533, rs6661105, rs669161, rs6703320, rs675828, rs6814242, rs6989344, rs7120590, rs7131676, rs7214164, rs747583, rs768255, rs768708, rs7828904, rs7899772, rs7900911, rs7925270, rs7975781, rs8111589, rs849084, rs873870, rs9386151, rs9504197, rs9690525, and rs9909561.

In some embodiments, the SNPs are selected from: rs10413687, rs10949838, rs1115649, rs11207002, rs11632601, rs11971741, rs12660563, rs13155942, rs1444647, rs1572801, rs17773922, rs1797700, rs1921681, rs1958312, rs196008, rs2001778, rs2323659, rs2427099, rs243992, rs251344, rs254264, rs2827530, rs290387, rs321949, rs348971, rs390316, rs3944117, rs425002, rs432586, rs444016, rs4453265, rs447247, rs4745577, rs484312, rs499946, rs500090, rs500399, rs505349, rs505662, rs516084, rs517316, rs517914, rs522810, rs531423, rs537330, rs539344, rs551372, rs567681, rs585487, rs600933, rs619208, rs622994, rs639298, rs642449, rs6700732, rs677866, rs683922, rs686851, rs6941942, rs7045684, rs7176924, rs7525374, rs870429, rs949312, rs9563831, rs970022, and rs985462.

In some embodiments, SNPs are selected from: rs1005241, rs1006101, rs10745725, rs10776856, rs10790342, rs11076499, rs11103233, rs11133637, rs11974817, rs12102203, rs12261, rs12460763, rs12543040, rs12695642, rs13137088, rs13139573, rs1327501, rs13438255, rs1360258, rs1421062, rs1432515, rs1452396, rs1518040, rs16853186, rs1712497, rs1792205, rs1863452, rs1991899, rs2022958, rs2099875, rs2108825, rs2132237, rs2195979, rs2248173, rs2250246, rs2268697, rs2270893, rs244887, rs2736966, rs2851428, rs2906237, rs2929724, rs3742257, rs3764584, rs3814332, rs4131376, rs4363444, rs4461567, rs4467511, rs4559013, rs4714802, rs4775899, rs4817609, rs488446, rs4950877, rs530913, rs6020434, rs6442703, rs6487229, rs6537064, rs654065, rs6576533, rs6661105, rs669161, rs6703320, rs675828, rs6814242, rs6989344, rs7120590, rs7131676, rs7214164, rs747583, rs768255, rs768708, rs7828904, rs7899772, rs7900911, rs7925270, rs7975781, rs8111589, rs849084, rs873870, rs9386151, rs9504197, rs9690525, and rs9909561.

The polymorphic targets can comprise one or more of any of the single nucleotide polymorphisms (SNPs) listed above and any combination thereof.

SNPs may be selected from any SNP provided herein or known in the art that meets any one or all of the criteria described herein for SNP selection. In some cases, SNPs can be located on any chromosome (e.g., chromosome 1, 2, 3, 4, 5, 6, 7, 8, 9, 10, 11, 12, 13, 14, 15, 16, 17, 18, 19, 20, 21, 22, X and/or Y). In some cases, SNPs can be located on autosomes (e.g., chromosome 1, 2, 3, 4, 5, 6, 7, 8, 9, 10, 11, 12, 13, 14, 15, 16, 17, 18, 19, 20, 21, 22), and not on chromosome X or chromosome Y. In some cases, SNPs can be located on certain autosomes (e.g., chromosome 1, 2, 3, 4, 5, 6, 7, 8, 9, 10, 11, 12, 14, 15, 16, 17, 19, 20, 22 and not chromosome 13, 18 or 22). In some cases, SNPs can be located on certain chromosomes suspected of having a genetic variation (e.g., aneuploidy), such as, for example, chromosome 13, 18, 21, X and/or Y (i.e., test chromosome(s)). In some cases, SNPs are located on a reference chromosome. In some cases, fetal fraction and the presence or absence of a genetic variation (e.g., aneuploidy) are determined simultaneously using a method provided herein.

In some embodiments, enriched (e.g., amplified) polymorphic nucleic acid targets are sequenced by a sequencing process. In some cases, the sequencing process is a sequencing by synthesis method, as described herein. Typically, sequencing by synthesis methods comprise a plurality of synthesis cycles, whereby a complementary nucleotide is added to a single stranded template and identified during each cycle. The number of cycles generally corresponds to read length. In some cases, polymorphic targets are selected such that a minimal read length (i.e., minimal number of cycles) is required to include amplification primer sequence and the polymorphic target site (e.g., SNP) in the read. In some cases, amplification primer sequence includes about 10 to about 30 nucleotides. For example, amplification primer sequence may include about 11, 12, 13, 14, 15, 16, 17, 18, 19, 20, 21, 22, 23, 24, 25, 26, 27, 28, or 29 nucleotides, in some embodiments. In some cases, amplification primer sequence includes about 20 nucleotides. In some embodiments, a SNP site is located within 1 nucleotide base position (i.e., adjacent to) to about 30 base positions from the 3′ terminus of an amplification primer. For example, a SNP site may be within 2, 3, 4, 5, 6, 7, 8, 9, 10, 11, 12, 13, 14, 15, 16, 17, 18, 19, 20, 21, 22, 23, 24, 25, 26, 27, 28, or 29 nucleotides of an amplification primer terminus. Read lengths can be any length that is inclusive of an amplification primer sequence and a polymorphic sequence or position. In some embodiments, read lengths can be about 10 nucleotides in length to about 50 nucleotides in length. For example, read lengths can be about 15, 20, 21, 22, 23, 24, 25, 26, 27, 28, 29, 30, 31, 32, 33, 34, 35, 36, 37, 38, 39, 40, or 45 nucleotides in length. In some cases, read length is about 36 nucleotides. In some cases, read length is about 27 nucleotides. Thus, in some cases, the sequencing by synthesis method comprises about 36 cycles and sometimes comprises about 27 cycles.

In some embodiments, a plurality of samples is sequenced in a single compartment (e.g., flow cell), which sometimes is referred to herein as sample multiplexing. Thus, in some embodiments, fetal fraction is determined for a plurality of samples in a multiplexed assay. For example, fetal fraction may be determined for about 10, 20, 30, 40, 50, 60, 70, 80, 90, 100, 200, 300, 400, 500, 600, 700, 800, 900, 1000, 2000 or more samples. In some cases, fetal fraction is determined for about 10 or more samples. In some cases, fetal fraction is determined for about 100 or more samples. In some cases, fetal fraction is determined for about 1000 or more samples.

### Methylation-Based Fetal Quantifier Assay

Determination of fetal nucleic acid content (e.g., fetal fraction) sometimes is performed using a methylation-based fetal quantifier assay (FQA) as described herein and, for example, in U.S. Patent Application Publication No. 2010/0105049, which is hereby incorporated by reference. This type of assay allows for the detection and quantification of fetal nucleic acid in a maternal sample based on the methylation status of the nucleic acid in the sample. In some cases, the amount of fetal nucleic acid from a maternal sample can be determined relative to the total amount of nucleic acid present, thereby providing the percentage of fetal nucleic acid in the sample. In some cases, the copy number of fetal nucleic acid can be determined in a maternal sample. In some cases, the amount of fetal nucleic acid can be determined in a sequence-specific (or locus-specific) manner and sometimes with sufficient sensitivity to allow for accurate chromosomal dosage analysis (for example, to detect the presence or absence of a fetal aneuploidy).

A fetal quantifier assay (FQA) can be performed in conjunction with any of the methods described herein. Such an assay can be performed by any method known in the art and/or described herein and in U.S. Patent Application Publication No. 2010/0105049, such as, for example, by a method that can distinguish between maternal and fetal DNA based on differential methylation status, and quantify (i.e. determine the amount of) the fetal DNA. Methods for differentiating nucleic acid based on methylation status include, but are not limited to, methylation sensitive capture, for example, using a MBD2-Fc fragment in which the methyl binding domain of MBD2 is fused to the Fc fragment of an antibody (MBD-FC) (Gebhard et al. (2006) Cancer Res. 66(12):6118-28); methylation specific antibodies; bisulfite conversion methods, for example, MSP (methylation-sensitive PCR), COBRA, methylation-sensitive single nucleotide primer extension (Ms-SNuPE) or Sequenom MassCLEAVE™ technology; and the use of methylation sensitive restriction enzymes (e.g., digestion of maternal DNA in a maternal sample using one or more methylation sensitive restriction enzymes thereby enriching the fetal DNA). Methyl-sensitive enzymes also can be used to differentiate nucleic acid based on methylation status, which, for example, can preferentially or substantially cleave or digest at their DNA recognition sequence if the latter is non-methylated. Thus, an unmethylated DNA sample will be cut into smaller fragments than a methylated DNA sample and a hypermethylated DNA sample will not be cleaved. Except where explicitly stated, any method for differentiating nucleic acid based on methylation status can be used with the compositions and methods of the technology herein. The amount of fetal DNA can be determined, for example, by introducing one or more competitors at known concentrations during an amplification reaction. Determining the amount of fetal DNA also can be done, for example, by RT-PCR, primer extension, sequencing and/or counting. In certain instances, the amount of nucleic acid can be determined using BEAMing technology as described in U.S. Patent Application Publication No. 2007/0065823. In some cases, the restriction efficiency can be determined and the efficiency rate is used to further determine the amount of fetal DNA.

In some cases, a fetal quantifier assay (FQA) can be used to determine the concentration of fetal DNA in a maternal sample, for example, by the following method: a) determine the total amount of DNA present in a maternal sample; b) selectively digest the maternal DNA in a maternal sample using one or more methylation sensitive restriction enzymes thereby enriching the fetal DNA; c) determine the amount of fetal DNA from step b); and d) compare the amount of fetal DNA from step c) to the total amount of DNA from step a), thereby determining the concentration of fetal DNA in the maternal sample. In some cases, the absolute copy number of fetal nucleic acid in a maternal sample can be determined, for example, using mass spectrometry and/or a system that uses a competitive PCR approach for absolute copy number measurements. See for example, Ding and Cantor (2003) Proc Natl Acad Sci USA 100:3059-3064, and U.S. Patent Application Publication No. 2004/0081993, both of which are hereby incorporated by reference.

Determining Fetal Nucleic Acid Content in Conjunction with Other Methods

The amount of fetal nucleic acid in extracellular nucleic acid (e.g., fetal fraction) can be quantified and used in conjunction with other methods for assessing a genetic variation (e.g., fetal aneuploidy, fetal gender). Thus, in certain embodiments, methods for determining the presence or absence of a genetic variation, for example, comprise an additional step of determining the amount of fetal nucleic acid. The amount of fetal nucleic acid can be determined in a nucleic acid sample from a subject before or after processing to prepare sample nucleic acid. In certain embodiments, the amount of fetal nucleic acid is determined in a sample after sample nucleic acid is processed and prepared, which amount is utilized for further assessment. In some embodiments, an outcome comprises factoring the fraction of fetal nucleic acid in the sample nucleic acid (e.g., adjusting counts, removing samples, making a call or not making a call).

The determination of fetal nucleic acid content (e.g., fetal fraction) can be performed before, during, at any one point in a method for assessing a genetic variation (e.g., aneuploidy detection, fetal gender determination), or after such methods. For example, to achieve a fetal gender or aneuploidy determination method with a given sensitivity or specificity, a fetal nucleic acid quantification method may be implemented prior to, during or after fetal gender or aneuploidy determination to identify those samples with greater than about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25% or more fetal nucleic acid. In some embodiments, samples determined as having a certain threshold amount of fetal nucleic acid (e.g., about 15% or more fetal nucleic acid; about 4% or more fetal nucleic acid) are further analyzed for fetal gender or aneuploidy determination, or the presence or absence of aneuploidy or genetic variation, for example. In certain embodiments, determinations of, for example, fetal gender or the presence or absence of aneuploidy are selected (e.g., selected and communicated to a patient) only for samples having a certain threshold amount of fetal nucleic acid (e.g., about 15% or more fetal nucleic acid; about 4% or more fetal nucleic acid).

### Additional Methods for Enriching for a Subpopulation of Nucleic Acid

In some embodiments, nucleic acid (e.g., extracellular nucleic acid) is enriched or relatively enriched for a subpopulation or species of nucleic acid. Nucleic acid subpopulations can include, for example, fetal nucleic acid, maternal nucleic acid, nucleic acid comprising fragments of a particular length or range of lengths, or nucleic acid from a particular genome region (e.g., single chromosome, set of chromosomes, and/or certain chromosome regions). Such enriched samples can be used in conjunction with the methods provided herein. Thus, in certain embodiments, methods of the technology herein comprise an additional step of enriching for a subpopulation of nucleic acid in a sample, such as, for example, fetal nucleic acid. In some cases, a method for determining fetal fraction described above also can be used to enrich for fetal nucleic acid. In certain embodiments, maternal nucleic acid is selectively removed (partially, substantially, almost completely or completely) from the sample. In some cases, enriching for a particular low copy number species nucleic acid (e.g., fetal nucleic acid) may improve quantitative sensitivity. Methods for enriching a sample for a particular species of nucleic acid are described herein and, for example, in U.S. Pat. No. 6,927,028, International Patent Application Publication No. WO2007/140417, International Patent Application Publication No. WO2007/147063, International Patent Application Publication No. WO2009/032779, International Patent Application Publication No. WO2009/032781, International Patent Application Publication No. WO2010/033639, International Patent Application Publication No. WO2011/034631, International Patent Application Publication No. WO2006/056480, and International Patent Application Publication No. WO2011/143659, all of which are incorporated by reference herein.

In some embodiments, nucleic acid is enriched for certain target fragment species and/or reference fragment species. In some cases, nucleic acid is enriched for a specific nucleic acid fragment length or range of fragment lengths using one or more length-based separation methods described below. In some cases, nucleic acid is enriched for fragments from a select genomic region (e.g., chromosome) using one or more sequence-based separation methods described herein and/or known in the art. Certain methods for enriching for a nucleic acid subpopulation (e.g., fetal nucleic acid) in a sample are described in detail below.

Some methods for enriching for a nucleic acid subpopulation (e.g., fetal nucleic acid) that can be used with the methods described herein include methods that exploit epigenetic differences between maternal and fetal nucleic acid. For example, fetal nucleic acid can be differentiated and separated from maternal nucleic acid based on methylation differences. Methylation-based fetal nucleic acid enrichment methods are described herein and, for example, in U.S. Patent Application Publication No. 2010/0105049, which is incorporated by reference herein. Such methods sometimes involve binding a sample nucleic acid to a methylation-specific binding agent (methyl-CpG binding protein (MBD), methylation specific antibodies, and the like) and separating bound nucleic acid from unbound nucleic acid based on differential methylation status. Such methods also can include the use of methylation-sensitive restriction enzymes (as described above; e.g., HhaI and HpaII), which allow for the enrichment of fetal nucleic acid regions in a maternal sample by selectively digesting nucleic acid from the maternal sample with an enzyme that selectively and completely or substantially digests the maternal nucleic acid to enrich the sample for at least one fetal nucleic acid region.

Another method for enriching for a nucleic acid subpopulation (e.g., fetal nucleic acid) that can be used with the methods described herein is a restriction endonuclease enhanced polymorphic sequence approach, such as a method described in U.S. Patent Application Publication No. 2009/0317818, which is incorporated by reference herein. Such methods include cleavage of nucleic acid comprising a non-target allele with a restriction endonuclease that recognizes the nucleic acid comprising the non-target allele but not the target allele; and amplification of uncleaved nucleic acid but not cleaved nucleic acid, where the uncleaved, amplified nucleic acid represents enriched target nucleic acid (e.g., fetal nucleic acid) relative to non-target nucleic acid (e.g., maternal nucleic acid). In some cases, nucleic acid may be selected such that it comprises an allele having a polymorphic site that is susceptible to selective digestion by a cleavage agent, for example.

Some methods for enriching for a nucleic acid subpopulation (e.g., fetal nucleic acid) that can be used with the methods described herein include selective enzymatic degradation approaches. Such methods involve protecting target sequences from exonuclease digestion thereby facilitating the elimination in a sample of undesired sequences (e.g., maternal DNA). For example, in one approach, sample nucleic acid is denatured to generate single stranded nucleic acid, single stranded nucleic acid is contacted with at least one target-specific primer pair under suitable annealing conditions, annealed primers are extended by nucleotide polymerization generating double stranded target sequences, and digesting single stranded nucleic acid using a nuclease that digests single stranded (i.e. non-target) nucleic acid. In some cases, the method can be repeated for at least one additional cycle. In some cases, the same target-specific primer pair is used to prime each of the first and second cycles of extension, and in some cases, different target-specific primer pairs are used for the first and second cycles.

Some methods for enriching for a nucleic acid subpopulation (e.g., fetal nucleic acid) that can be used with the methods described herein include massively parallel signature sequencing (MPSS) approaches. MPSS typically is a solid phase method that uses adapter (i.e. tag) ligation, followed by adapter decoding, and reading of the nucleic acid sequence in small increments. Tagged PCR products are typically amplified such that each nucleic acid generates a PCR product with a unique tag. Tags are often used to attach the PCR products to microbeads. After several rounds of ligation-based sequence determination, for example, a sequence signature can be identified from each bead. Each signature sequence (MPSS tag) in a MPSS dataset is analyzed, compared with all other signatures, and all identical signatures are counted.

In some cases, certain MPSS-based enrichment methods can include amplification (e.g., PCR)-based approaches. In some cases, loci-specific amplification methods can be used (e.g., using loci-specific amplification primers). In some cases, a multiplex SNP allele PCR approach can be used. In some cases, a multiplex SNP allele PCR approach can be used in combination with uniplex sequencing. For example, such an approach can involve the use of multiplex PCR (e.g., MASSARRAY system) and incorporation of capture probe sequences into the amplicons followed by sequencing using, for example, the Illumina MPSS system. In some cases, a multiplex SNP allele PCR approach can be used in combination with a three-primer system and indexed sequencing. For example, such an approach can involve the use of multiplex PCR (e.g., MASSARRAY system) with primers having a first capture probe incorporated into certain loci-specific forward PCR primers and adapter sequences incorporated into loci-specific reverse PCR primers, to thereby generate amplicons, followed by a secondary PCR to incorporate reverse capture sequences and molecular index barcodes for sequencing using, for example, the Illumina MPSS system. In some cases, a multiplex SNP allele PCR approach can be used in combination with a four-primer system and indexed sequencing. For example, such an approach can involve the use of multiplex PCR (e.g., MASSARRAY system) with primers having adaptor sequences incorporated into both loci-specific forward and loci-specific reverse PCR primers, followed by a secondary PCR to incorporate both forward and reverse capture sequences and molecular index barcodes for sequencing using, for example, the Illumina MPSS system. In some cases, a microfluidics approach can be used. In some cases, an array-based microfluidics approach can be used. For example, such an approach can involve the use of a microfluidics array (e.g., Fluidigm) for amplification at low plex and incorporation of index and capture probes, followed by sequencing. In some cases, an emulsion microfluidics approach can be used, such as, for example, digital droplet PCR.

In some cases, universal amplification methods can be used (e.g., using universal or non-loci-specific amplification primers). In some cases, universal amplification methods can be used in combination with pull-down approaches. In some cases, the method can include biotinylated ultramer pull-down (e.g., biotinylated pull-down assays from Agilent or IDT) from a universally amplified sequencing library. For example, such an approach can involve preparation of a standard library, enrichment for selected regions by a pull-down assay, and a secondary universal amplification step. In some cases, pull-down approaches can be used in combination with ligation-based methods. In some cases, the method can include biotinylated ultramer pull down with sequence specific adapter ligation (e.g., HALOPLEX PCR, Halo Genomics). For example, such an approach can involve the use of selector probes to capture restriction enzyme-digested fragments, followed by ligation of captured products to an adaptor, and universal amplification followed by sequencing. In some cases, pull-down approaches can be used in combination with extension and ligation-based methods. In some cases, the method can include molecular inversion probe (MIP) extension and ligation. For example, such an approach can involve the use of molecular inversion probes in combination with sequence adapters followed by universal amplification and sequencing. In some cases, complementary DNA can be synthesized and sequenced without amplification.

In some cases, extension and ligation approaches can be performed without a pull-down component. In some cases, the method can include loci-specific forward and reverse primer hybridization, extension and ligation. Such methods can further include universal amplification or complementary DNA synthesis without amplification, followed by sequencing. Such methods can reduce or exclude background sequences during analysis, in some cases.

In some cases, pull-down approaches can be used with an optional amplification component or with no amplification component. In some cases, the method can include a modified pull-down assay and ligation with full incorporation of capture probes without universal amplification. For example, such an approach can involve the use of modified selector probes to capture restriction enzyme-digested fragments, followed by ligation of captured products to an adaptor, optional amplification, and sequencing. In some cases, the method can include a biotinylated pull-down assay with extension and ligation of adaptor sequence in combination with circular single stranded ligation. For example, such an approach can involve the use of selector probes to capture regions of interest (i.e. target sequences), extension of the probes, adaptor ligation, single stranded circular ligation, optional amplification, and sequencing. In some cases, the analysis of the sequencing result can separate target sequences form background.

In some embodiments, nucleic acid is enriched for fragments from a select genomic region (e.g., chromosome) using one or more sequence-based separation methods described herein. Sequence-based separation generally is based on nucleotide sequences present in the fragments of interest (e.g., target and/or reference fragments) and substantially not present in other fragments of the sample or present in an insubstantial amount of the other fragments (e.g., 5% or less). In some embodiments, sequence-based separation can generate separated target fragments and/or separated reference fragments. Separated target fragments and/or separated reference fragments typically are isolated away from the remaining fragments in the nucleic acid sample. In some cases, the separated target fragments and the separated reference fragments also are isolated away from each other (e.g., isolated in separate assay compartments). In some cases, the separated target fragments and the separated reference fragments are isolated together (e.g., isolated in the same assay compartment). In some embodiments, unbound fragments can be differentially removed or degraded or digested.

In some embodiments, a selective nucleic acid capture process is used to separate target and/or reference fragments away from the nucleic acid sample. Commercially available nucleic acid capture systems include, for example, Nimblegen sequence capture system (Roche NimbleGen, Madison, WI); Illumina BEADARRAY platform (Illumina, San Diego, CA); Affymetrix GENECHIP platform (Affymetrix, Santa Clara, CA); Agilent SureSelect Target Enrichment System (Agilent Technologies, Santa Clara, CA); and related platforms. Such methods typically involve hybridization of a capture oligonucleotide to a portion or all of the nucleotide sequence of a target or reference fragment and can include use of a solid phase (e.g., solid phase array) and/or a solution based platform. Capture oligonucleotides (sometimes referred to as “bait”) can be selected or designed such that they preferentially hybridize to nucleic acid fragments from selected genomic regions or loci (e.g., one of chromosomes 21, 18, 13, X or Y, or a reference chromosome).

In some embodiments, nucleic acid is enriched for a particular nucleic acid fragment length, range of lengths, or lengths under or over a particular threshold or cutoff using one or more length-based separation methods. Nucleic acid fragment length typically refers to the number of nucleotides in the fragment. Nucleic acid fragment length also is sometimes referred to as nucleic acid fragment size. In some embodiments, a length-based separation method is performed without measuring lengths of individual fragments. In some embodiments, a length based separation method is performed in conjunction with a method for determining length of individual fragments. In some embodiments, length-based separation refers to a size fractionation procedure where all or part of the fractionated pool can be isolated (e.g., retained) and/or analyzed. Size fractionation procedures are known in the art (e.g., separation on an array, separation by a molecular sieve, separation by gel electrophoresis, separation by column chromatography (e.g., size-exclusion columns), and microfluidics-based approaches). In some cases, length-based separation approaches can include fragment circularization, chemical treatment (e.g., formaldehyde, polyethylene glycol (PEG)), mass spectrometry and/or size-specific nucleic acid amplification, for example.

Certain length-based separation methods that can be used with methods described herein employ a selective sequence tagging approach, for example. In such methods, a fragment size species (e.g., short fragments) nucleic acids are selectively tagged in a sample that includes long and short nucleic acids. Such methods typically involve performing a nucleic acid amplification reaction using a set of nested primers which include inner primers and outer primers. In some cases, one or both of the inner can be tagged to thereby introduce a tag onto the target amplification product. The outer primers generally do not anneal to the short fragments that carry the (inner) target sequence. The inner primers can anneal to the short fragments and generate an amplification product that carries a tag and the target sequence. Typically, tagging of the long fragments is inhibited through a combination of mechanisms which include, for example, blocked extension of the inner primers by the prior annealing and extension of the outer primers. Enrichment for tagged fragments can be accomplished by any of a variety of methods, including for example, exonuclease digestion of single stranded nucleic acid and amplification of the tagged fragments using amplification primers specific for at least one tag.

Another length-based separation method that can be used with methods described herein involves subjecting a nucleic acid sample to polyethylene glycol (PEG) precipitation. Examples of methods include those described in International Patent Application Publication Nos. WO2007/140417 and WO2010/115016. This method in general entails contacting a nucleic acid sample with PEG in the presence of one or more monovalent salts under conditions sufficient to substantially precipitate large nucleic acids without substantially precipitating small (e.g., less than 300 nucleotides) nucleic acids.

Another size-based enrichment method that can be used with methods described herein involves circularization by ligation, for example, using circligase. Short nucleic acid fragments typically can be circularized with higher efficiency than long fragments. Non-circularized sequences can be separated from circularized sequences, and the enriched short fragments can be used for further analysis.

### Nucleic Acid Amplification and Detection

Following separation of nucleic acid in a methylation-differential manner, nucleic acid may be amplified and/or subjected to a detection process (e.g., sequence-based analysis, mass spectrometry). Furthermore, once it is determined that one particular genomic sequence of fetal origin is hypermethylated or hypomethylated compared to the maternal counterpart, the amount of this fetal genomic sequence can be determined. Subsequently, this amount can be compared to a standard control value and serve as an indication for the potential of certain pregnancy-associated disorder.

Nucleotide sequences, or amplified nucleic acid sequences, or detectable products prepared from the foregoing, can be detected by a suitable detection process. Non-limiting examples of methods of detection, quantification, sequencing and the like include mass detection of mass modified amplicons (e.g., matrix-assisted laser desorption ionization (MALDI) mass spectrometry and electrospray (ES) mass spectrometry), a primer extension method (e.g., iPLEX™; Sequenom, Inc.), direct DNA sequencing, Molecular Inversion Probe (MIP) technology from Affymetrix, restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Invader assay, hybridization using at least one probe, hybridization using at least one fluorescently labeled probe, cloning and sequencing, electrophoresis, the use of hybridization probes and quantitative real time polymerase chain reaction (QRT-PCR), digital PCR, nanopore sequencing, chips and combinations thereof. In some embodiments the amount of each amplified nucleic acid species is determined by mass spectrometry, primer extension, sequencing (e.g., any suitable method, for example nanopore or pyrosequencing), Quantitative PCR (Q-PCR or QRT-PCR), digital PCR, combinations thereof, and the like.

Nucleic acid detection and/or quantification also may include, for example, solid support array based detection of fluorescently labeled nucleic acid with fluorescent labels incorporated during or after PCR, single molecule detection of fluorescently labeled molecules in solution or captured on a solid phase, or other sequencing technologies such as, for example, sequencing using ION TORRENT or MISEQ platforms or single molecule sequencing technologies using instrumentation such as, for example, PACBIO sequencers, HELICOS sequencer, or nanopore sequencing technologies.

In some cases, nucleotide sequences, or amplified nucleic acid sequences, or detectable products prepared from the foregoing, are detected using a sequencing process (e.g., such as a sequencing process described herein). Nucleic acid quantifications generated by a method comprising a sequencing detection process may be compared to nucleic acid quantifications generated by a method comprising a different detection process (e.g., mass spectrometry). Such comparisons may be expressed using an R² value, which is a measure of correlation between two outcomes (e.g., nucleic acid quantifications). In some cases, nucleic acid quantifications (e.g., fetal copy number quantifications) are highly correlated (i.e., have high R² values) for quantifications generated using different detection processes (e.g., sequencing and mass spectrometry). In some cases, R² values for nucleic acid quantifications generated using different detection processes may be between about 0.90 and about 1.0. For example, R² values may be about 0.91, 0.92, 0.93, 0.94, 0.95, 0.96, 0.97, 0.98, or 0.99.

### Amplification of Nucleotide Sequences

In many instances, it is desirable to amplify a nucleic acid sequence of the technology herein using any of several nucleic acid amplification procedures which are well known in the art (listed above and described in greater detail below). Specifically, nucleic acid amplification is the enzymatic synthesis of nucleic acid amplicons (copies) which contain a sequence that is complementary to a nucleic acid sequence being amplified. Nucleic acid amplification is especially beneficial when the amount of target sequence present in a sample is very low. By amplifying the target sequences and detecting the amplicon synthesized, the sensitivity of an assay can be vastly improved, since fewer target sequences are needed at the beginning of the assay to better ensure detection of nucleic acid in the sample belonging to the organism or virus of interest.

A variety of polynucleotide amplification methods are well established and frequently used in research. For instance, the general methods of polymerase chain reaction (PCR) for polynucleotide sequence amplification are well known in the art and are thus not described in detail herein. For a review of PCR methods, protocols, and principles in designing primers, see, e.g., Innis, et al., PCR Protocols: A Guide to Methods and Applications, Academic Press, Inc. N.Y., 1990. PCR reagents and protocols are also available from commercial vendors, such as Roche Molecular Systems.

PCR is most usually carried out as an automated process with a thermostable enzyme. In this process, the temperature of the reaction mixture is cycled through a denaturing region, a primer annealing region, and an extension reaction region automatically. Machines specifically adapted for this purpose are commercially available.

Although PCR amplification of a polynucleotide sequence is typically used in practicing the present technology, one of skill in the art will recognize that the amplification of a genomic sequence found in a maternal blood sample may be accomplished by any known method, such as ligase chain reaction (LCR), transcription-mediated amplification, and self-sustained sequence replication or nucleic acid sequence-based amplification (NASBA), each of which provides sufficient amplification. More recently developed branched-DNA technology may also be used to qualitatively demonstrate the presence of a particular genomic sequence of the technology herein, which represents a particular methylation pattern, or to quantitatively determine the amount of this particular genomic sequence in the maternal blood. For a review of branched-DNA signal amplification for direct quantitation of nucleic acid sequences in clinical samples, see Nolte, Adv. Clin. Chem. 33:201-235, 1998.

The compositions and processes of the technology herein are also particularly useful when practiced with digital PCR. Digital PCR was first developed by Kalinina and colleagues (Kalinina et al., “Nanoliter scale PCR with TaqMan detection.” Nucleic Acids Research. 25; 1999-2004, (1997)) and further developed by Vogelstein and Kinzler (Digital PCR. Proc Natl Acad Sci USA. 96; 9236-41, (1999)). The application of digital PCR for use with fetal diagnostics was first described by Cantor et al. (PCT Patent Publication No. WO05023091A2) and subsequently described by Quake et al. (US Patent Publication No. US 20070202525), which are both hereby incorporated by reference. Digital PCR takes advantage of nucleic acid (DNA, cDNA or RNA) amplification on a single molecule level, and offers a highly sensitive method for quantifying low copy number nucleic acid. Fluidigm® Corporation offers systems for the digital analysis of nucleic acids.

The terms “amplify”, “amplification”, “amplification reaction”, or “amplifying” refer to any in vitro process for multiplying the copies of a nucleic acid. Amplification sometimes refers to an “exponential” increase in nucleic acid. However, “amplifying” as used herein can also refer to linear increases in the numbers of a select nucleic acid, but is different than a one-time, single primer extension step. In some embodiments a limited amplification reaction, also known as pre-amplification, can be performed. Pre-amplification is a method in which a limited amount of amplification occurs due to a small number of cycles, for example 10 cycles, being performed. Pre-amplification can allow some amplification, but stops amplification prior to the exponential phase, and typically produces about 500 copies of the desired nucleotide sequence(s). Use of pre-amplification may also limit inaccuracies associated with depleted reactants in standard PCR reactions, for example, and also may reduce amplification biases due to nucleotide sequence or abundance of the nucleic acid. In some embodiments a one-time primer extension may be performed as a prelude to linear or exponential amplification.

Any suitable amplification technique can be utilized. Amplification of polynucleotides include, but are not limited to, polymerase chain reaction (PCR); ligation amplification (or ligase chain reaction (LCR)); amplification methods based on the use of Q-beta replicase or template-dependent polymerase (see US Patent Publication Number US20050287592); helicase-dependant isothermal amplification (Vincent et al., “Helicase-dependent isothermal DNA amplification”. EMBO reports 5 (8): 795-800 (2004)); strand displacement amplification (SDA); thermophilic SDA nucleic acid sequence based amplification (3SR or NASBA) and transcription-associated amplification (TAA). Non-limiting examples of PCR amplification methods include standard PCR, AFLP-PCR, Allele-specific PCR, Alu-PCR, Asymmetric PCR, Colony PCR, Hot start PCR, Inverse PCR (IPCR), In situ PCR (ISH), Intersequence-specific PCR (ISSR-PCR), Long PCR, Multiplex PCR, Nested PCR, Quantitative PCR, Reverse Transcriptase PCR (RT-PCR), Real Time PCR, Single cell PCR, Solid phase PCR, digital PCR, combinations thereof, and the like. For example, amplification can be accomplished using digital PCR, in certain embodiments (see e.g. Kalinina et al., “Nanoliter scale PCR with TaqMan detection.” Nucleic Acids Research. 25; 1999-2004, (1997); Vogelstein and Kinzler (Digital PCR. Proc Natl Acad Sci USA. 96; 9236-41, (1999); PCT Patent Publication No. WO05023091A2; US Patent Publication No. US 20070202525). Digital PCR takes advantage of nucleic acid (DNA, cDNA or RNA) amplification on a single molecule level, and offers a highly sensitive method for quantifying low copy number nucleic acid. Systems for digital amplification and analysis of nucleic acids are available (e.g., Fluidigm® Corporation). Reagents and hardware for conducting PCR are commercially available.

A generalized description of an amplification process is presented herein. Primers and nucleic acid are contacted, and complementary sequences anneal to one another, for example. Primers can anneal to a nucleic acid, at or near (e.g., adjacent to, abutting, and the like) a sequence of interest. In some embodiments, the primers in a set hybridize within about 10 to 30 nucleotides from a nucleic acid sequence of interest and produce amplified products. In some embodiments, the primers hybridize within the nucleic acid sequence of interest.

A reaction mixture, containing components necessary for enzymatic functionality, is added to the primer-nucleic acid hybrid, and amplification can occur under suitable conditions. Components of an amplification reaction may include, but are not limited to, e.g., primers (e.g., individual primers, primer pairs, primer sets and the like) a polynucleotide template, polymerase, nucleotides, dNTPs and the like. In some embodiments, non-naturally occurring nucleotides or nucleotide analogs, such as analogs containing a detectable label (e.g., fluorescent or colorimetric label), may be used for example. Polymerases can be selected by a person of ordinary skill and include polymerases for thermocycle amplification (e.g., Taq DNA Polymerase; Q-Bio™ Taq DNA Polymerase (recombinant truncated form of Taq DNA Polymerase lacking 5′-3′exo activity); SurePrime™ Polymerase (chemically modified Taq DNA polymerase for “hot start” PCR); Arrow™ Taq DNA Polymerase (high sensitivity and long template amplification)) and polymerases for thermostable amplification (e.g., RNA polymerase for transcription-mediated amplification (TMA) described at World Wide Web URL “gen-probe.com/pdfs/tma_whiteppr.pdf”). Other enzyme components can be added, such as reverse transcriptase for transcription mediated amplification (TMA) reactions, for example.

PCR conditions can be dependent upon primer sequences, abundance of nucleic acid, and the desired amount of amplification, and therefore, one of skill in the art may choose from a number of PCR protocols available (see, e.g., U.S. Pat. Nos. 4,683,195 and 4,683,202; and PCR Protocols: A Guide to Methods and Applications, Innis et al., eds, 1990. Digital PCR is also known in the art; see, e.g., United States Patent Application Publication no. 20070202525, filed Feb. 2, 2007, which is hereby incorporated by reference). PCR is typically carried out as an automated process with a thermostable enzyme. In this process, the temperature of the reaction mixture is cycled through a denaturing step, a primer-annealing step, and an extension reaction step automatically. Some PCR protocols also include an activation step and a final extension step. Machines specifically adapted for this purpose are commercially available. A non-limiting example of a PCR protocol that may be suitable for embodiments described herein is, treating the sample at 95° C. for 5 minutes; repeating thirty-five cycles of 95° C. for 45 seconds and 68° C. for 30 seconds; and then treating the sample at 72° C. for 3 minutes. A completed PCR reaction can optionally be kept at 4° C. until further action is desired. Multiple cycles frequently are performed using a commercially available thermal cycler. Suitable isothermal amplification processes known and selected by the person of ordinary skill in the art also may be applied, in certain embodiments.

In some embodiments, an amplification product may include naturally occurring nucleotides, non-naturally occurring nucleotides, nucleotide analogs and the like and combinations of the foregoing. An amplification product often has a nucleotide sequence that is identical to or substantially identical to a nucleic acid sequence herein, or complement thereof. A “substantially identical” nucleotide sequence in an amplification product will generally have a high degree of sequence identity to the nucleotide sequence species being amplified or complement thereof (e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater than 99% sequence identity), and variations sometimes are a result of infidelity of the polymerase used for extension and/or amplification, or additional nucleotide sequence(s) added to the primers used for amplification.

### Primers

Primers useful for detection, amplification, quantification, sequencing and analysis of nucleic acid are provided. The term “primer” as used herein refers to a nucleic acid that includes a nucleotide sequence capable of hybridizing or annealing to a target nucleic acid, at or near (e.g., adjacent to) a specific region of interest. Primers can allow for specific determination of a target nucleic acid nucleotide sequence or detection of the target nucleic acid (e.g., presence or absence of a sequence or copy number of a sequence), or feature thereof, for example. A primer may be naturally occurring or synthetic. The term “specific” or “specificity”, as used herein, refers to the binding or hybridization of one molecule to another molecule, such as a primer for a target polynucleotide. That is, “specific” or “specificity” refers to the recognition, contact, and formation of a stable complex between two molecules, as compared to substantially less recognition, contact, or complex formation of either of those two molecules with other molecules. As used herein, the term “anneal” refers to the formation of a stable complex between two molecules. The terms “primer”, “oligo”, or “oligonucleotide” may be used interchangeably throughout the document, when referring to primers.

A primer nucleic acid can be designed and synthesized using suitable processes, and may be of any length suitable for hybridizing to a nucleotide sequence of interest (e.g., where the nucleic acid is in liquid phase or bound to a solid support) and performing analysis processes described herein. Primers may be designed based upon a target nucleotide sequence. A primer in some embodiments may be about 10 to about 100 nucleotides, about 10 to about 70 nucleotides, about 10 to about 50 nucleotides, about 15 to about 30 nucleotides, or about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 nucleotides in length. A primer may be composed of naturally occurring and/or non-naturally occurring nucleotides (e.g., labeled nucleotides), or a mixture thereof. Primers suitable for use with embodiments described herein, may be synthesized and labeled using known techniques. Primers may be chemically synthesized according to the solid phase phosphoramidite triester method first described by Beaucage and Caruthers, Tetrahedron Letts., 22:1859-1862, 1981, using an automated synthesizer, as described in Needham-VanDevanter et al., Nucleic Acids Res. 12:6159-6168, 1984. Purification of primers can be effected by native acrylamide gel electrophoresis or by anion-exchange high-performance liquid chromatography (HPLC), for example, as described in Pearson and Regnier, J. Chrom., 255:137-149, 1983.

All or a portion of a primer nucleic acid sequence (naturally occurring or synthetic) may be substantially complementary to a target nucleic acid, in some embodiments. As referred to herein, “substantially complementary” with respect to sequences refers to nucleotide sequences that will hybridize with each other. The stringency of the hybridization conditions can be altered to tolerate varying amounts of sequence mismatch. Included are target and primer sequences that are 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other.

Primers that are substantially complimentary to a target nucleic acid sequence are also substantially identical to the compliment of the target nucleic acid sequence. That is, primers are substantially identical to the anti-sense strand of the nucleic acid. As referred to herein, “substantially identical” with respect to sequences refers to nucleotide sequences that are 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more identical to each other. One test for determining whether two nucleotide sequences are substantially identical is to determine the percent of identical nucleotide sequences shared.

Primer sequences and length may affect hybridization to target nucleic acid sequences. Depending on the degree of mismatch between the primer and target nucleic acid, low, medium or high stringency conditions may be used to effect primer/target annealing. As used herein, the term “stringent conditions” refers to conditions for hybridization and washing. Methods for hybridization reaction temperature condition optimization are known to those of skill in the art, and may be found in Current Protocols in Molecular Biology, John Wiley & Sons, N.Y., 6.3.1-6.3.6 (1989). Aqueous and non-aqueous methods are described in that reference and either can be used. Non-limiting examples of stringent hybridization conditions are hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C. Stringent hybridization temperatures can also be altered (i.e. lowered) with the addition of certain organic solvents, formamide for example. Organic solvents, like formamide, reduce the thermal stability of double-stranded polynucleotides, so that hybridization can be performed at lower temperatures, while still maintaining stringent conditions and extending the useful life of nucleic acids that may be heat labile. Features of primers can be applied to probes and oligonucleotides, such as, for example, the competitive and inhibitory oligonucleotides provided herein.

As used herein, the phrase “hybridizing” or grammatical variations thereof, refers to binding of a first nucleic acid molecule to a second nucleic acid molecule under low, medium or high stringency conditions, or under nucleic acid synthesis conditions. Hybridizing can include instances where a first nucleic acid molecule binds to a second nucleic acid molecule, where the first and second nucleic acid molecules are complementary. As used herein, “specifically hybridizes” refers to preferential hybridization under nucleic acid synthesis conditions of a primer, to a nucleic acid molecule having a sequence complementary to the primer compared to hybridization to a nucleic acid molecule not having a complementary sequence. For example, specific hybridization includes the hybridization of a primer to a target nucleic acid sequence that is complementary to the primer.

In some embodiments primers can include a nucleotide subsequence that may be complementary to a solid phase nucleic acid primer hybridization sequence or substantially complementary to a solid phase nucleic acid primer hybridization sequence (e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater than 99% identical to the primer hybridization sequence complement when aligned). A primer may contain a nucleotide subsequence not complementary to or not substantially complementary to a solid phase nucleic acid primer hybridization sequence (e.g., at the 3′ or 5′ end of the nucleotide subsequence in the primer complementary to or substantially complementary to the solid phase primer hybridization sequence).

A primer, in certain embodiments, may contain a modification such as one or more inosines, abasic sites, locked nucleic acids, minor groove binders, duplex stabilizers (e.g., acridine, spermidine), Tm modifiers or any modifier that changes the binding properties of the primers or probes. A primer, in certain embodiments, may contain a detectable molecule or entity (e.g., a fluorophore, radioisotope, colorimetric agent, particle, enzyme and the like, as described above for labeled competitor oligonucleotides).

A primer also may refer to a polynucleotide sequence that hybridizes to a subsequence of a target nucleic acid or another primer and facilitates the detection of a primer, a target nucleic acid or both, as with molecular beacons, for example. The term “molecular beacon” as used herein refers to detectable molecule, where the detectable property of the molecule is detectable only under certain specific conditions, thereby enabling it to function as a specific and informative signal. Non-limiting examples of detectable properties are, optical properties, electrical properties, magnetic properties, chemical properties and time or speed through an opening of known size.

In some embodiments, the primers are complementary to genomic DNA target sequences. In some cases, the forward and reverse primers hybridize to the 5′ and 3′ ends of the genomic DNA target sequences. In some embodiments, primers that hybridize to the genomic DNA target sequences also hybridize to competitor oligonucleotides that were designed to compete with corresponding genomic DNA target sequences for binding of the primers. In some cases, the primers hybridize or anneal to the genomic DNA target sequences and the corresponding competitor oligonucleotides with the same or similar hybridization efficiencies. In some cases the hybridization efficiencies are different. The ratio between genomic DNA target amplicons and competitor amplicons can be measured during the reaction. For example if the ratio is 1:1 at 28 cycles but 2:1 at 35, this could indicate that during the end of the amplification reaction the primers for one target (i.e. genomic DNA target or competitor) are either reannealing faster than the other, or the denaturation is less effective than the other.

In some embodiments primers are used in sets. As used herein, an amplification primer set is one or more pairs of forward and reverse primers for a given region. Thus, for example, primers that amplify genomic targets for region 1 (i.e. targets 1a and 1b) are considered a primer set. Primers that amplify genomic targets for region 2 (i.e. targets 2a and 2b) are considered a different primer set. In some embodiments, the primer sets that amplify targets within a particular region also amplify the corresponding competitor oligonucleotide(s). A plurality of primer pairs may constitute a primer set in certain embodiments (e.g., about 2, 3, 4, 5, 6, 7, 8, 9, 10, 15, 20, 25, 30, 35, 40, 45, 50, 55, 60, 65, 70, 75, 80, 85, 90, 95 or 100 pairs). In some embodiments a plurality of primer sets, each set comprising pair(s) of primers, may be used.

### Determination of Polynucleotide Sequences

Techniques for polynucleotide sequence determination are also well established and widely practiced in the relevant research field. For instance, the basic principles and general techniques for polynucleotide sequencing are described in various research reports and treatises on molecular biology and recombinant genetics, such as Wallace et al., supra; Sambrook and Russell, supra, and Ausubel et al., supra. DNA sequencing methods routinely practiced in research laboratories, either manual or automated, can be used for practicing the present technology. Additional means suitable for detecting changes in a polynucleotide sequence for practicing the methods of the present technology include but are not limited to mass spectrometry, primer extension, polynucleotide hybridization, real-time PCR, and electrophoresis.

Use of a primer extension reaction also can be applied in methods of the technology herein. A primer extension reaction operates, for example, by discriminating the SNP alleles by the incorporation of deoxynucleotides and/or dideoxynucleotides to a primer extension primer which hybridizes to a region adjacent to the SNP site. The primer is extended with a polymerase. The primer extended SNP can be detected physically by mass spectrometry or by a tagging moiety such as biotin. As the SNP site is only extended by a complementary deoxynucleotide or dideoxynucleotide that is either tagged by a specific label or generates a primer extension product with a specific mass, the SNP alleles can be discriminated and quantified.

Reverse transcribed and amplified nucleic acids may be modified nucleic acids. Modified nucleic acids can include nucleotide analogs, and in certain embodiments include a detectable label and/or a capture agent. Examples of detectable labels include without limitation fluorophores, radioisotopes, colormetric agents, light emitting agents, chemiluminescent agents, light scattering agents, enzymes and the like. Examples of capture agents include without limitation an agent from a binding pair selected from antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B12/intrinsic factor, chemical reactive group/complementary chemical reactive group (e.g., sulfhydryl/maleimide, sulfhydryl/haloacetyl derivative, amine/isotriocyanate, amine/succinimidyl ester, and amine/sulfonyl halides) pairs, and the like. Modified nucleic acids having a capture agent can be immobilized to a solid support in certain embodiments

Mass spectrometry is a particularly effective method for the detection of a polynucleotide of the technology herein, for example a PCR amplicon, a primer extension product or a detector probe that is cleaved from a target nucleic acid. The presence of the polynucleotide sequence is verified by comparing the mass of the detected signal with the expected mass of the polynucleotide of interest. The relative signal strength, e.g., mass peak on a spectra, for a particular polynucleotide sequence indicates the relative population of a specific allele, thus enabling calculation of the allele ratio directly from the data. For a review of genotyping methods using Sequenom® standard iPLEX™ assay and MassARRAY® technology, see Jurinke, C., Oeth, P., van den Boom, D., “MALDI-TOF mass spectrometry: a versatile tool for high-performance DNA analysis.” Mol. Biotechnol. 26, 147-164 (2004); and Oeth, P. et al., “iPLEX™ Assay: Increased Plexing Efficiency and Flexibility for MassARRAY® System through single base primer extension with mass-modified Terminators.” SEQUENOM Application Note (2005), both of which are hereby incorporated by reference. For a review of detecting and quantifying target nucleic using cleavable detector probes that are cleaved during the amplification process and detected by mass spectrometry, see U.S. patent application Ser. No. 11/950,395, which was filed Dec. 4, 2007, and is hereby incorporated by reference.

Sequencing technologies are improving in terms of throughput and cost. Sequencing technologies, such as that achievable on the 454 platform (Roche) (Margulies, M. et al. 2005 Nature 437, 376-380), Illumina Genome Analyzer (or Solexa platform) or SOLiD System (Applied Biosystems) or the Helicos True Single Molecule DNA sequencing technology (Harris T D et al. 2008 Science, 320, 106-109), the single molecule, real-time (SMRT™) technology of Pacific Biosciences, and nanopore sequencing (Soni G V and Meller A. 2007 Clin Chem 53: 1996-2001), allow the sequencing of many nucleic acid molecules isolated from a specimen at high orders of multiplexing in a parallel fashion (Dear Brief Funct Genomic Proteomic 2003; 1: 397-416).

Each of these platforms allow sequencing of clonally expanded or non-amplified single molecules of nucleic acid fragments. Certain platforms involve, for example, (i) sequencing by ligation of dye-modified probes (including cyclic ligation and cleavage), (ii) pyrosequencing, and (iii) single-molecule sequencing. Nucleotide sequence species, amplification nucleic acid species and detectable products generated there from can be considered a “study nucleic acid” for purposes of analyzing a nucleotide sequence by such sequence analysis platforms.

Sequencing by ligation is a nucleic acid sequencing method that relies on the sensitivity of DNA ligase to base-pairing mismatch. DNA ligase joins together ends of DNA that are correctly base paired. Combining the ability of DNA ligase to join together only correctly base paired DNA ends, with mixed pools of fluorescently labeled oligonucleotides or primers, enables sequence determination by fluorescence detection. Longer sequence reads may be obtained by including primers containing cleavable linkages that can be cleaved after label identification. Cleavage at the linker removes the label and regenerates the 5′ phosphate on the end of the ligated primer, preparing the primer for another round of ligation. In some embodiments primers may be labeled with more than one fluorescent label (e.g., 1 fluorescent label, 2, 3, or 4 fluorescent labels). An example of a system that can be used by a person of ordinary skill based on sequencing by ligation generally involves the following steps. Clonal bead populations can be prepared in emulsion microreactors containing study nucleic acid (“template”), amplification reaction components, beads and primers. After amplification, templates are denatured and bead enrichment is performed to separate beads with extended templates from undesired beads (e.g., beads with no extended templates). The template on the selected beads undergoes a 3′ modification to allow covalent bonding to the slide, and modified beads can be deposited onto a glass slide. Deposition chambers offer the ability to segment a slide into one, four or eight chambers during the bead loading process. For sequence analysis, primers hybridize to the adapter sequence. A set of four color dye-labeled probes competes for ligation to the sequencing primer. Specificity of probe ligation is achieved by interrogating every 4th and 5th base during the ligation series. Five to seven rounds of ligation, detection and cleavage record the color at every 5th position with the number of rounds determined by the type of library used. Following each round of ligation, a new complimentary primer offset by one base in the 5′ direction is laid down for another series of ligations. Primer reset and ligation rounds (5-7 ligation cycles per round) are repeated sequentially five times to generate 25-35 base pairs of sequence for a single tag. With mate-paired sequencing, this process is repeated for a second tag. Such a system can be used to exponentially amplify amplification products generated by a process described herein, e.g., by ligating a heterologous nucleic acid to the first amplification product generated by a process described herein and performing emulsion amplification using the same or a different solid support originally used to generate the first amplification product. Such a system also may be used to analyze amplification products directly generated by a process described herein by bypassing an exponential amplification process and directly sorting the solid supports described herein on the glass slide.

Pyrosequencing is a nucleic acid sequencing method based on sequencing by synthesis, which relies on detection of a pyrophosphate released on nucleotide incorporation. Generally, sequencing by synthesis involves synthesizing, one nucleotide at a time, a DNA strand complimentary to the strand whose sequence is being sought. Study nucleic acids may be immobilized to a solid support, hybridized with a sequencing primer, incubated with DNA polymerase, ATP sulfurylase, luciferase, apyrase, adenosine 5′ phosphsulfate and luciferin. Nucleotide solutions are sequentially added and removed. Correct incorporation of a nucleotide releases a pyrophosphate, which interacts with ATP sulfurylase and produces ATP in the presence of adenosine 5′ phosphsulfate, fueling the luciferin reaction, which produces a chemiluminescent signal allowing sequence determination.

An example of a system that can be used by a person of ordinary skill based on pyrosequencing generally involves the following steps: ligating an adaptor nucleic acid to a study nucleic acid and hybridizing the study nucleic acid to a bead; amplifying a nucleotide sequence in the study nucleic acid in an emulsion; sorting beads using a picoliter multiwell solid support; and sequencing amplified nucleotide sequences by pyrosequencing methodology (e.g., Nakano et al., “Single-molecule PCR using water-in-oil emulsion;” Journal of Biotechnology 102: 117-124 (2003)). Such a system can be used to exponentially amplify amplification products generated by a process described herein, e.g., by ligating a heterologous nucleic acid to the first amplification product generated by a process described herein.

Certain single-molecule sequencing embodiments are based on the principal of sequencing by synthesis, and utilize single-pair Fluorescence Resonance Energy Transfer (single pair FRET) as a mechanism by which photons are emitted as a result of successful nucleotide incorporation. The emitted photons often are detected using intensified or high sensitivity cooled charge-couple-devices in conjunction with total internal reflection microscopy (TIRM). Photons are only emitted when the introduced reaction solution contains the correct nucleotide for incorporation into the growing nucleic acid chain that is synthesized as a result of the sequencing process. In FRET based single-molecule sequencing, energy is transferred between two fluorescent dyes, sometimes polymethine cyanine dyes Cy3 and Cy5, through long-range dipole interactions. The donor is excited at its specific excitation wavelength and the excited state energy is transferred, non-radiatively to the acceptor dye, which in turn becomes excited. The acceptor dye eventually returns to the ground state by radiative emission of a photon. The two dyes used in the energy transfer process represent the “single pair”, in single pair FRET. Cy3 often is used as the donor fluorophore and often is incorporated as the first labeled nucleotide. Cy5 often is used as the acceptor fluorophore and is used as the nucleotide label for successive nucleotide additions after incorporation of a first Cy3 labeled nucleotide. The fluorophores generally are within 10 nanometers of each for energy transfer to occur successfully.

An example of a system that can be used based on single-molecule sequencing generally involves hybridizing a primer to a study nucleic acid to generate a complex; associating the complex with a solid phase; iteratively extending the primer by a nucleotide tagged with a fluorescent molecule; and capturing an image of fluorescence resonance energy transfer signals after each iteration (e.g., U.S. Pat. No. 7,169,314; Braslavsky et al., PNAS 100(7): 3960-3964 (2003)). Such a system can be used to directly sequence amplification products generated by processes described herein. In some embodiments the released linear amplification product can be hybridized to a primer that contains sequences complementary to immobilized capture sequences present on a solid support, a bead or glass slide for example. Hybridization of the primer—released linear amplification product complexes with the immobilized capture sequences, immobilizes released linear amplification products to solid supports for single pair FRET based sequencing by synthesis. The primer often is fluorescent, so that an initial reference image of the surface of the slide with immobilized nucleic acids can be generated. The initial reference image is useful for determining locations at which true nucleotide incorporation is occurring. Fluorescence signals detected in array locations not initially identified in the “primer only” reference image are discarded as non-specific fluorescence. Following immobilization of the primer—released linear amplification product complexes, the bound nucleic acids often are sequenced in parallel by the iterative steps of, a) polymerase extension in the presence of one fluorescently labeled nucleotide, b) detection of fluorescence using appropriate microscopy, TIRM for example, c) removal of fluorescent nucleotide, and d) return to step a with a different fluorescently labeled nucleotide.

In some embodiments, nucleotide sequencing may be by solid phase single nucleotide sequencing methods and processes. Solid phase single nucleotide sequencing methods involve contacting sample nucleic acid and solid support under conditions in which a single molecule of sample nucleic acid hybridizes to a single molecule of a solid support. Such conditions can include providing the solid support molecules and a single molecule of sample nucleic acid in a “microreactor.” Such conditions also can include providing a mixture in which the sample nucleic acid molecule can hybridize to solid phase nucleic acid on the solid support. Single nucleotide sequencing methods useful in the embodiments described herein are described in U.S. Provisional Patent Application Ser. No. 61/021,871 filed Jan. 17, 2008.

In certain embodiments, nanopore sequencing detection methods include (a) contacting a nucleic acid for sequencing (“base nucleic acid,” e.g., linked probe molecule) with sequence-specific detectors, under conditions in which the detectors specifically hybridize to substantially complementary subsequences of the base nucleic acid; (b) detecting signals from the detectors and (c) determining the sequence of the base nucleic acid according to the signals detected. In certain embodiments, the detectors hybridized to the base nucleic acid are disassociated from the base nucleic acid (e.g., sequentially dissociated) when the detectors interfere with a nanopore structure as the base nucleic acid passes through a pore, and the detectors disassociated from the base sequence are detected. In some embodiments, a detector disassociated from a base nucleic acid emits a detectable signal, and the detector hybridized to the base nucleic acid emits a different detectable signal or no detectable signal. In certain embodiments, nucleotides in a nucleic acid (e.g., linked probe molecule) are substituted with specific nucleotide sequences corresponding to specific nucleotides (“nucleotide representatives”), thereby giving rise to an expanded nucleic acid (e.g., U.S. Pat. No. 6,723,513), and the detectors hybridize to the nucleotide representatives in the expanded nucleic acid, which serves as a base nucleic acid. In such embodiments, nucleotide representatives may be arranged in a binary or higher order arrangement (e.g., Soni and Meller, Clinical Chemistry 53(11): 1996-2001 (2007)). In some embodiments, a nucleic acid is not expanded, does not give rise to an expanded nucleic acid, and directly serves a base nucleic acid (e.g., a linked probe molecule serves as a non-expanded base nucleic acid), and detectors are directly contacted with the base nucleic acid. For example, a first detector may hybridize to a first subsequence and a second detector may hybridize to a second subsequence, where the first detector and second detector each have detectable labels that can be distinguished from one another, and where the signals from the first detector and second detector can be distinguished from one another when the detectors are disassociated from the base nucleic acid. In certain embodiments, detectors include a region that hybridizes to the base nucleic acid (e.g., two regions), which can be about 3 to about 100 nucleotides in length (e.g., about 4, 5, 6, 7, 8, 9, 10, 11, 12, 13, 14, 15, 16, 17, 18, 19, 20, 25, 30, 35, 40, 50, 55, 60, 65, 70, 75, 80, 85, 90, or 95 nucleotides in length). A detector also may include one or more regions of nucleotides that do not hybridize to the base nucleic acid. In some embodiments, a detector is a molecular beacon. A detector often comprises one or more detectable labels independently selected from those described herein. Each detectable label can be detected by any convenient detection process capable of detecting a signal generated by each label (e.g., magnetic, electric, chemical, optical and the like). For example, a CD camera can be used to detect signals from one or more distinguishable quantum dots linked to a detector.

In certain sequence analysis embodiments, reads may be used to construct a larger nucleotide sequence, which can be facilitated by identifying overlapping sequences in different reads and by using identification sequences in the reads. Such sequence analysis methods and software for constructing larger sequences from reads are known to the person of ordinary skill (e.g., Venter et al., Science 291: 1304-1351 (2001)). Specific reads, partial nucleotide sequence constructs, and full nucleotide sequence constructs may be compared between nucleotide sequences within a sample nucleic acid (i.e., internal comparison) or may be compared with a reference sequence (i.e., reference comparison) in certain sequence analysis embodiments. Internal comparisons sometimes are performed in situations where a sample nucleic acid is prepared from multiple samples or from a single sample source that contains sequence variations. Reference comparisons sometimes are performed when a reference nucleotide sequence is known and an objective is to determine whether a sample nucleic acid contains a nucleotide sequence that is substantially similar or the same, or different, than a reference nucleotide sequence. Sequence analysis is facilitated by sequence analysis apparatus and components known to the person of ordinary skill in the art.

Methods provided herein allow for high-throughput detection of nucleic acid species in a plurality of nucleic acids (e.g., nucleotide sequence species, amplified nucleic acid species and detectable products generated from the foregoing). Multiplexing refers to the simultaneous detection of more than one nucleic acid species. General methods for performing multiplexed reactions in conjunction with mass spectrometry, are known (see, e.g., U.S. Pat. Nos. 6,043,031, 5,547,835 and International PCT application No. WO 97/37041). Multiplexing provides an advantage that a plurality of nucleic acid species (e.g., some having different sequence variations) can be identified in as few as a single mass spectrum, as compared to having to perform a separate mass spectrometry analysis for each individual target nucleic acid species. Methods provided herein lend themselves to high-throughput, highly-automated processes for analyzing sequence variations with high speed and accuracy, in some embodiments. In some embodiments, methods herein may be multiplexed at high levels in a single reaction.

In certain embodiments, the number of nucleic acid species multiplexed include, without limitation, about 1 to about 500 (e.g., about 1-3, 3-5, 5-7, 7-9, 9-11, 11-13, 13-15, 15-17, 17-19, 19-21, 21-23, 23-25, 25-27, 27-29, 29-31, 31-33, 33-35, 35-37, 37-39, 39-41, 41-43, 43-45, 45-47, 47-49, 49-51, 51-53, 53-55, 55-57, 57-59, 59-61, 61-63, 63-65, 65-67, 67-69, 69-71, 71-73, 73-75, 75-77, 77-79, 79-81, 81-83, 83-85, 85-87, 87-89, 89-91, 91-93, 93-95, 95-97, 97-101, 101-103, 103-105, 105-107, 107-109, 109-111, 111-113, 113-115, 115-117, 117-119, 121-123, 123-125, 125-127, 127-129, 129-131, 131-133, 133-135, 135-137, 137-139, 139-141, 141-143, 143-145, 145-147, 147-149, 149-151, 151-153, 153-155, 155-157, 157-159, 159-161, 161-163, 163-165, 165-167, 167-169, 169-171, 171-173, 173-175, 175-177, 177-179, 179-181, 181-183, 183-185, 185-187, 187-189, 189-191, 191-193, 193-195, 195-197, 197-199, 199-201, 201-203, 203-205, 205-207, 207-209, 209-211, 211-213, 213-215, 215-217, 217-219, 219-221, 221-223, 223-225, 225-227, 227-229, 229-231, 231-233, 233-235, 235-237, 237-239, 239-241, 241-243, 243-245, 245-247, 247-249, 249-251, 251-253, 253-255, 255-257, 257-259, 259-261, 261-263, 263-265, 265-267, 267-269, 269-271, 271-273, 273-275, 275-277, 277-279, 279-281, 281-283, 283-285, 285-287, 287-289, 289-291, 291-293, 293-295, 295-297, 297-299, 299-301, 301-303, 303-305, 305-307, 307-309, 309-311, 311-313, 313-315, 315-317, 317-319, 319-321, 321-323, 323-325, 325-327, 327-329, 329-331, 331-333, 333-335, 335-337, 337-339, 339-341, 341-343, 343-345, 345-347, 347-349, 349-351, 351-353, 353-355, 355-357, 357-359, 359-361, 361-363, 363-365, 365-367, 367-369, 369-371, 371-373, 373-375, 375-377, 377-379, 379-381, 381-383, 383-385, 385-387, 387-389, 389-391, 391-393, 393-395, 395-397, 397-401, 401-403, 403-405, 405-407, 407-409, 409-411, 411-413, 413-415, 415-417, 417-419, 419-421, 421-423, 423-425, 425-427, 427-429, 429-431, 431-433, 433-435, 435-437, 437-439, 439-441, 441-443, 443-445, 445-447, 447-449, 449-451, 451-453, 453-455, 455-457, 457-459, 459-461, 461-463, 463-465, 465-467, 467-469, 469-471, 471-473, 473-475, 475-477, 477-479, 479-481, 481-483, 483-485, 485-487, 487-489, 489-491, 491-493, 493-495, 495-497, 497-501).

Design methods for achieving resolved mass spectra with multiplexed assays can include primer and oligonucleotide design methods and reaction design methods. See, for example, the multiplex schemes provided in Tables X and Y. For primer and oligonucleotide design in multiplexed assays, the same general guidelines for primer design applies for uniplexed reactions, such as avoiding false priming and primer dimers, only more primers are involved for multiplex reactions. For mass spectrometry applications, analyte peaks in the mass spectra for one assay are sufficiently resolved from a product of any assay with which that assay is multiplexed, including pausing peaks and any other by-product peaks. Also, analyte peaks optimally fall within a user-specified mass window, for example, within a range of 5,000-8,500 Da. In some embodiments multiplex analysis may be adapted to mass spectrometric detection of chromosome abnormalities, for example. In certain embodiments multiplex analysis may be adapted to various single nucleotide or nanopore based sequencing methods described herein. Commercially produced micro-reaction chambers or devices or arrays or chips may be used to facilitate multiplex analysis, and are commercially available.

### Additional Methods for Obtaining Nucleotide Sequence Reads

In some embodiments, nucleic acids (e.g., nucleic acid fragments, sample nucleic acid, cell-free nucleic acid) may be sequenced. In some cases, a full or substantially full sequence is obtained and sometimes a partial sequence is obtained. Sequencing, mapping and related analytical methods are known in the art (e.g., United States Patent Application Publication US2009/0029377, incorporated by reference). Certain aspects of such processes are described hereafter.

As used herein, “reads” are short nucleotide sequences produced by any sequencing process described herein or known in the art. Reads can be generated from one end of nucleic acid fragments (“single-end reads”), and sometimes are generated from both ends of nucleic acids (“double-end reads”). In certain embodiments, “obtaining” nucleic acid sequence reads of a sample from a subject and/or “obtaining” nucleic acid sequence reads of a biological specimen from one or more reference persons can involve directly sequencing nucleic acid to obtain the sequence information. In some embodiments, “obtaining” can involve receiving sequence information obtained directly from a nucleic acid by another.

In some embodiments, one nucleic acid sample from one individual is sequenced. In certain embodiments, nucleic acid samples from two or more biological samples, where each biological sample is from one individual or two or more individuals, are pooled and the pool is sequenced. In the latter embodiments, a nucleic acid sample from each biological sample often is identified by one or more unique identification tags.

In some embodiments, a fraction of the genome is sequenced, which sometimes is expressed in the amount of the genome covered by the determined nucleotide sequences (e.g., “fold” coverage less than 1). When a genome is sequenced with about 1-fold coverage, roughly 100% of the nucleotide sequence of the genome is represented by reads. A genome also can be sequenced with redundancy, where a given region of the genome can be covered by two or more reads or overlapping reads (e.g., “fold” coverage greater than 1). In some embodiments, a genome is sequenced with about 0.1-fold to about 100-fold coverage, about 0.2-fold to 20-fold coverage, or about 0.2-fold to about 1-fold coverage (e.g., about 0.2-, 0.3-, 0.4-, 0.5-, 0.6-, 0.7-, 0.8-, 0.9-, 1-, 2-, 3-, 4-, 5-, 6-, 7-, 8-, 9-, 10-, 15-, 20-, 30-, 40-, 50-, 60-, 70-, 80-, 90-fold coverage).

In certain embodiments, a fraction of a nucleic acid pool that is sequenced in a run is further sub-selected prior to sequencing. In certain embodiments, hybridization-based techniques (e.g., using oligonucleotide arrays) can be used to first sub-select for nucleic acid sequences from certain chromosomes (e.g., a potentially aneuploid chromosome and other chromosome(s) not involved in the aneuploidy tested). In some embodiments, nucleic acid can be fractionated by size (e.g., by gel electrophoresis, size exclusion chromatography or by microfluidics-based approach) and in certain instances, fetal nucleic acid can be enriched by selecting for nucleic acid having a lower molecular weight (e.g., less than 300 base pairs, less than 200 base pairs, less than 150 base pairs, less than 100 base pairs). In some embodiments, fetal nucleic acid can be enriched by suppressing maternal background nucleic acid, such as by the addition of formaldehyde. In some embodiments, a portion or subset of a pre-selected pool of nucleic acids is sequenced randomly.

In some embodiments, the nucleic acid is amplified prior to sequencing. In some embodiments, a portion or subset of the nucleic acid is amplified prior to sequencing. In some cases, a sequencing library is prepared prior to or during a sequencing process. Methods for preparing a sequencing library are known in the art and commercially available platforms may be used for certain applications. Certain commercially available library platforms may be compatible with certain nucleotide sequencing processes described herein. For example, one or more commercially available library platforms may be compatible with a sequencing by synthesis process. In some cases, a ligation-based library preparation method is used (e.g., ILLUMINA TRUSEQ, Illumina, San Diego CA). Ligation-based library preparation methods typically use a methylated adaptor design which can incorporate an index sequence at the initial ligation step and often can be used to prepare samples for single-read sequencing, paired-end sequencing and multiplexed sequencing. In some cases, a transposon-based library preparation method is used (e.g., EPICENTRE NEXTERA, Epicentre, Madison WI). Transposon-based methods typically use in vitro transposition to simultaneously fragment and tag DNA in a single-tube reaction (often allowing incorporation of platform-specific tags and optional barcodes), and prepare sequencer-ready libraries.

Any sequencing method suitable for conducting methods described herein can be utilized. In some embodiments, a high-throughput sequencing method is used. High-throughput sequencing methods generally involve clonally amplified DNA templates or single DNA molecules that are sequenced in a massively parallel fashion within a flow cell (e.g. as described in Metzker M Nature Rev 11:31-46 (2010); Volkerding et al. Clin Chem 55:641-658 (2009)). Such sequencing methods also can provide digital quantitative information, where each sequence read is a countable “sequence tag” or “count” representing an individual clonal DNA template or a single DNA molecule. High-throughput sequencing technologies include, for example, sequencing-by-synthesis with reversible dye terminators, sequencing by oligonucleotide probe ligation, pyrosequencing and real time sequencing.

Systems utilized for high-throughput sequencing methods are commercially available and include, for example, the Roche 454 platform, the Applied Biosystems SOLID platform, the Helicos True Single Molecule DNA sequencing technology, the sequencing-by-hybridization platform from Affymetrix Inc., the single molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and nanopore sequencing also can be used in high-throughput sequencing approaches.

In some embodiments, first generation technology, such as, for example, Sanger sequencing including the automated Sanger sequencing, can be used in the methods provided herein. Additional sequencing technologies that include the use of developing nucleic acid imaging technologies (e.g. transmission electron microscopy (TEM) and atomic force microscopy (AFM)), also are contemplated herein. Examples of various sequencing technologies are described below.

A nucleic acid sequencing technology that may be used in the methods described herein is sequencing-by-synthesis and reversible terminator-based sequencing (e.g. Illumina's Genome Analyzer; Genome Analyzer II; HISEQ 2000; HISEQ 2500 (Illumina, San Diego CA)). With this technology, millions of nucleic acid (e.g. DNA) fragments can be sequenced in parallel. In one example of this type of sequencing technology, a flow cell is used which contains an optically transparent slide with 8 individual lanes on the surfaces of which are bound oligonucleotide anchors (e.g., adaptor primers). A flow cell often is a solid support that can be configured to retain and/or allow the orderly passage of reagent solutions over bound analytes. Flow cells frequently are planar in shape, optically transparent, generally in the millimeter or sub-millimeter scale, and often have channels or lanes in which the analyte/reagent interaction occurs.

In certain sequencing by synthesis procedures, for example, template DNA (e.g., circulating cell-free DNA (ccfDNA)) sometimes is fragmented into lengths of several hundred base pairs in preparation for library generation. In some embodiments, library preparation can be performed without further fragmentation or size selection of the template DNA (e.g., ccfDNA). Sample isolation and library generation may be performed using automated methods and apparatus, in certain embodiments. Briefly, template DNA is end repaired by a fill-in reaction, exonuclease reaction or a combination of a fill-in reaction and exonuclease reaction. The resulting blunt-end repaired template DNA is extended by a single nucleotide, which is complementary to a single nucleotide overhang on the 3′ end of an adapter primer, and often increases ligation efficiency. Any complementary nucleotides can be used for the extension/overhang nucleotides (e.g., A/T, C/G), however adenine frequently is used to extend the end-repaired DNA, and thymine often is used as the 3′ end overhang nucleotide.

In certain sequencing by synthesis procedures, for example, adapter oligonucleotides are complementary to the flow-cell anchors, and sometimes are utilized to associate the modified template DNA (e.g., end-repaired and single nucleotide extended) with a solid support, such as the inside surface of a flow cell, for example. In some embodiments, the adapter also includes identifiers (i.e., indexing nucleotides, or “barcode” nucleotides (e.g., a unique sequence of nucleotides usable as an identifier to allow unambiguous identification of a sample and/or chromosome)), one or more sequencing primer hybridization sites (e.g., sequences complementary to universal sequencing primers, single end sequencing primers, paired end sequencing primers, multiplexed sequencing primers, and the like), or combinations thereof (e.g., adapter/sequencing, adapter/identifier, adapter/identifier/sequencing). Identifiers or nucleotides contained in an adapter often are six or more nucleotides in length, and frequently are positioned in the adaptor such that the identifier nucleotides are the first nucleotides sequenced during the sequencing reaction. In certain embodiments, identifier nucleotides are associated with a sample but are sequenced in a separate sequencing reaction to avoid compromising the quality of sequence reads. Subsequently, the reads from the identifier sequencing and the DNA template sequencing are linked together and the reads de-multiplexed. After linking and de-multiplexing the sequence reads and/or identifiers can be further adjusted or processed as described herein.

In certain sequencing by synthesis procedures, utilization of identifiers allows multiplexing of sequence reactions in a flow cell lane, thereby allowing analysis of multiple samples per flow cell lane. The number of samples that can be analyzed in a given flow cell lane often is dependent on the number of unique identifiers utilized during library preparation and/or probe design. Non limiting examples of commercially available multiplex sequencing kits include Illumina's multiplexing sample preparation oligonucleotide kit and multiplexing sequencing primers and PhiX control kit (e.g., Illumina's catalog numbers PE-400-1001 and PE-400-1002, respectively). The methods described herein can be performed using any number of unique identifiers (e.g., 4, 8, 12, 24, 48, 96, or more). The greater the number of unique identifiers, the greater the number of samples and/or chromosomes, for example, that can be multiplexed in a single flow cell lane. Multiplexing using 12 identifiers, for example, allows simultaneous analysis of 96 samples (e.g., equal to the number of wells in a 96 well microwell plate) in an 8 lane flow cell. Similarly, multiplexing using 48 identifiers, for example, allows simultaneous analysis of 384 samples (e.g., equal to the number of wells in a 384 well microwell plate) in an 8 lane flow cell.

In certain sequencing by synthesis procedures, adapter-modified, single-stranded template DNA is added to the flow cell and immobilized by hybridization to the anchors under limiting-dilution conditions. In contrast to emulsion PCR, DNA templates are amplified in the flow cell by “bridge” amplification, which relies on captured DNA strands “arching” over and hybridizing to an adjacent anchor oligonucleotide. Multiple amplification cycles convert the single-molecule DNA template to a clonally amplified arching “cluster,” with each cluster containing approximately 1000 clonal molecules. Approximately 50×10⁶ separate clusters can be generated per flow cell. For sequencing, the clusters are denatured, and a subsequent chemical cleavage reaction and wash leave only forward strands for single-end sequencing. Sequencing of the forward strands is initiated by hybridizing a primer complementary to the adapter sequences, which is followed by addition of polymerase and a mixture of four differently colored fluorescent reversible dye terminators. The terminators are incorporated according to sequence complementarity in each strand in a clonal cluster. After incorporation, excess reagents are washed away, the clusters are optically interrogated, and the fluorescence is recorded. With successive chemical steps, the reversible dye terminators are unblocked, the fluorescent labels are cleaved and washed away, and the next sequencing cycle is performed. This iterative, sequencing-by-synthesis process sometimes requires approximately 2.5 days to generate read lengths of 36 bases. With 50×10⁶ clusters per flow cell, the overall sequence output can be greater than 1 billion base pairs (Gb) per analytical run.

Another nucleic acid sequencing technology that may be used with the methods described herein is 454 sequencing (Roche). 454 sequencing uses a large-scale parallel pyrosequencing system capable of sequencing about 400-600 megabases of DNA per run. The process typically involves two steps. In the first step, sample nucleic acid (e.g. DNA) is sometimes fractionated into smaller fragments (300-800 base pairs) and polished (made blunt at each end). Short adaptors are then ligated onto the ends of the fragments. These adaptors provide priming sequences for both amplification and sequencing of the sample-library fragments. One adaptor (Adaptor B) contains a 5′-biotin tag for immobilization of the DNA library onto streptavidin-coated beads. After nick repair, the non-biotinylated strand is released and used as a single-stranded template DNA (sstDNA) library. The sstDNA library is assessed for its quality and the optimal amount (DNA copies per bead) needed for emPCR is determined by titration. The sstDNA library is immobilized onto beads. The beads containing a library fragment carry a single sstDNA molecule. The bead-bound library is emulsified with the amplification reagents in a water-in-oil mixture. Each bead is captured within its own microreactor where PCR amplification occurs. This results in bead-immobilized, clonally amplified DNA fragments.

In the second step of 454 sequencing, single-stranded template DNA library beads are added to an incubation mix containing DNA polymerase and are layered with beads containing sulfurylase and luciferase onto a device containing pico-liter sized wells. Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing exploits the release of pyrophosphate (PPi) upon nucleotide addition. PPi is converted to ATP by ATP sulfurylase in the presence of adenosine 5′ phosphosulfate. Luciferase uses ATP to convert luciferin to oxyluciferin, and this reaction generates light that is discerned and analyzed (see, for example, Margulies, M. et al.

### Nature 437:376-380 (2005)).

Another nucleic acid sequencing technology that may be used in the methods provided herein is Applied Biosystems' SOLiD™ technology. In SOLiD™ sequencing-by-ligation, a library of nucleic acid fragments is prepared from the sample and is used to prepare clonal bead populations. With this method, one species of nucleic acid fragment will be present on the surface of each bead (e.g. magnetic bead). Sample nucleic acid (e.g. genomic DNA) is sheared into fragments, and adaptors are subsequently attached to the 5′ and 3′ ends of the fragments to generate a fragment library. The adapters are typically universal adapter sequences so that the starting sequence of every fragment is both known and identical. Emulsion PCR takes place in microreactors containing all the necessary reagents for PCR. The resulting PCR products attached to the beads are then covalently bound to a glass slide. Primers then hybridize to the adapter sequence within the library template. A set of four fluorescently labeled di-base probes compete for ligation to the sequencing primer. Specificity of the di-base probe is achieved by interrogating every 1st and 2nd base in each ligation reaction. Multiple cycles of ligation, detection and cleavage are performed with the number of cycles determining the eventual read length. Following a series of ligation cycles, the extension product is removed and the template is reset with a primer complementary to the n-1 position for a second round of ligation cycles. Often, five rounds of primer reset are completed for each sequence tag. Through the primer reset process, each base is interrogated in two independent ligation reactions by two different primers. For example, the base at read position 5 is assayed by primer number 2 in ligation cycle 2 and by primer number 3 in ligation cycle 1.

Another nucleic acid sequencing technology that may be used in the methods described herein is the Helicos True Single Molecule Sequencing (tSMS). In the tSMS technique, a polyA sequence is added to the 3′ end of each nucleic acid (e.g. DNA) strand from the sample. Each strand is labeled by the addition of a fluorescently labeled adenosine nucleotide. The DNA strands are then hybridized to a flow cell, which contains millions of oligo-T capture sites that are immobilized to the flow cell surface. The templates can be at a density of about 100 million templates/cm². The flow cell is then loaded into a sequencing apparatus and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are detected by imaging the flow cell surface. After imaging, a cleavage step removes the fluorescent label, and the process is repeated with other fluorescently labeled nucleotides until the desired read length is achieved. Sequence information is collected with each nucleotide addition step (see, for example, Harris T. D. et al., Science 320:106-109 (2008)).

Another nucleic acid sequencing technology that may be used in the methods provided herein is the single molecule, real-time (SMRT™) sequencing technology of Pacific Biosciences. With this method, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in an out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is then repeated.

Another nucleic acid sequencing technology that may be used in the methods described herein is ION TORRENT (Life Technologies) single molecule sequencing which pairs semiconductor technology with a simple sequencing chemistry to directly translate chemically encoded information (A, C, G, T) into digital information (0, 1) on a semiconductor chip. ION TORRENT uses a high-density array of micro-machined wells to perform nucleic acid sequencing in a massively parallel way. Each well holds a different DNA molecule. Beneath the wells is an ion-sensitive layer and beneath that an ion sensor. Typically, when a nucleotide is incorporated into a strand of DNA by a polymerase, a hydrogen ion is released as a byproduct. If a nucleotide, for example a C, is added to a DNA template and is then incorporated into a strand of DNA, a hydrogen ion will be released. The charge from that ion will change the pH of the solution, which can be detected by an ion sensor. A sequencer can call the base, going directly from chemical information to digital information. The sequencer then sequentially floods the chip with one nucleotide after another. If the next nucleotide that floods the chip is not a match, no voltage change will be recorded and no base will be called. If there are two identical bases on the DNA strand, the voltage will be double, and the chip will record two identical bases called. Because this is direct detection (i.e. detection without scanning, cameras or light), each nucleotide incorporation is recorded in seconds.

Another nucleic acid sequencing technology that may be used in the methods described herein is the chemical-sensitive field effect transistor (CHEMFET) array. In one example of this sequencing technique, DNA molecules are placed into reaction chambers, and the template molecules can be hybridized to a sequencing primer bound to a polymerase. Incorporation of one or more triphosphates into a new nucleic acid strand at the 3′ end of the sequencing primer can be detected by a change in current by a CHEMFET sensor. An array can have multiple CHEMFET sensors. In another example, single nucleic acids are attached to beads, and the nucleic acids can be amplified on the bead, and the individual beads can be transferred to individual reaction chambers on a CHEMFET array, with each chamber having a CHEMFET sensor, and the nucleic acids can be sequenced (see, for example, U.S. Patent Application Publication No. 2009/0026082).

Another nucleic acid sequencing technology that may be used in the methods described herein is electron microscopy. In one example of this sequencing technique, individual nucleic acid (e.g. DNA) molecules are labeled using metallic labels that are distinguishable using an electron microscope. These molecules are then stretched on a flat surface and imaged using an electron microscope to measure sequences (see, for example, Moudrianakis E. N. and Beer M. Proc Natl Acad Sci USA. 1965 March; 53:564-71). In some cases, transmission electron microscopy (TEM) is used (e.g. Halcyon Molecular's TEM method). This method, termed Individual Molecule Placement Rapid Nano Transfer (IMPRNT), includes utilizing single atom resolution transmission electron microscope imaging of high-molecular weight (e.g. about 150 kb or greater) DNA selectively labeled with heavy atom markers and arranging these molecules on ultra-thin films in ultra-dense (3 nm strand-to-strand) parallel arrays with consistent base-to-base spacing. The electron microscope is used to image the molecules on the films to determine the position of the heavy atom markers and to extract base sequence information from the DNA (see, for example, International Patent Application No. WO 2009/046445).

Other sequencing methods that may be used to conduct methods herein include digital PCR and sequencing by hybridization. Digital polymerase chain reaction (digital PCR or dPCR) can be used to directly identify and quantify nucleic acids in a sample. Digital PCR can be performed in an emulsion, in some embodiments. For example, individual nucleic acids are separated, e.g., in a microfluidic chamber device, and each nucleic acid is individually amplified by PCR. Nucleic acids can be separated such that there is no more than one nucleic acid per well. In some embodiments, different probes can be used to distinguish various alleles (e.g. fetal alleles and maternal alleles). Alleles can be enumerated to determine copy number. In sequencing by hybridization, the method involves contacting a plurality of polynucleotide sequences with a plurality of polynucleotide probes, where each of the plurality of polynucleotide probes can be optionally tethered to a substrate. The substrate can be a flat surface with an array of known nucleotide sequences, in some embodiments. The pattern of hybridization to the array can be used to determine the polynucleotide sequences present in the sample. In some embodiments, each probe is tethered to a bead, e.g., a magnetic bead or the like. Hybridization to the beads can be identified and used to identify the plurality of polynucleotide sequences within the sample.

In some embodiments, nanopore sequencing can be used in the methods described herein. Nanopore sequencing is a single-molecule sequencing technology whereby a single nucleic acid molecule (e.g. DNA) is sequenced directly as it passes through a nanopore. A nanopore is a small hole or channel, of the order of 1 nanometer in diameter. Certain transmembrane cellular proteins can act as nanopores (e.g. alpha-hemolysin). In some cases, nanopores can be synthesized (e.g. using a silicon platform). Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree and generates characteristic changes to the current. The amount of current which can pass through the nanopore at any given moment therefore varies depending on whether the nanopore is blocked by an A, a C, a G, a T, or in some cases, methyl-C. The change in the current through the nanopore as the DNA molecule passes through the nanopore represents a direct reading of the DNA sequence. In some cases a nanopore can be used to identify individual DNA bases as they pass through the nanopore in the correct order (see, for example, Soni G V and Meller A. Clin Chem 53: 1996-2001 (2007); International Patent Application No. WO2010/004265).

There are a number of ways that nanopores can be used to sequence nucleic acid molecules. In some embodiments, an exonuclease enzyme, such as a deoxyribonuclease, is used. In this case, the exonuclease enzyme is used to sequentially detach nucleotides from a nucleic acid (e.g. DNA) molecule. The nucleotides are then detected and discriminated by the nanopore in order of their release, thus reading the sequence of the original strand. For such an embodiment, the exonuclease enzyme can be attached to the nanopore such that a proportion of the nucleotides released from the DNA molecule is capable of entering and interacting with the channel of the nanopore. The exonuclease can be attached to the nanopore structure at a site in close proximity to the part of the nanopore that forms the opening of the channel. In some cases, the exonuclease enzyme can be attached to the nanopore structure such that its nucleotide exit trajectory site is orientated towards the part of the nanopore that forms part of the opening.

In some embodiments, nanopore sequencing of nucleic acids involves the use of an enzyme that pushes or pulls the nucleic acid (e.g. DNA) molecule through the pore. In this case, the ionic current fluctuates as a nucleotide in the DNA molecule passes through the pore. The fluctuations in the current are indicative of the DNA sequence. For such an embodiment, the enzyme can be attached to the nanopore structure such that it is capable of pushing or pulling the target nucleic acid through the channel of a nanopore without interfering with the flow of ionic current through the pore. The enzyme can be attached to the nanopore structure at a site in close proximity to the part of the structure that forms part of the opening. The enzyme can be attached to the subunit, for example, such that its active site is orientated towards the part of the structure that forms part of the opening.

In some embodiments, nanopore sequencing of nucleic acids involves detection of polymerase bi-products in close proximity to a nanopore detector. In this case, nucleoside phosphates (nucleotides) are labeled so that a phosphate labeled species is released upon the addition of a polymerase to the nucleotide strand and the phosphate labeled species is detected by the pore. Typically, the phosphate species contains a specific label for each nucleotide. As nucleotides are sequentially added to the nucleic acid strand, the bi-products of the base addition are detected. The order that the phosphate labeled species are detected can be used to determine the sequence of the nucleic acid strand.

The length of the sequence read is often associated with the particular sequencing technology. High-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, the sequence reads are of a mean, median or average length of about 15 bp to 900 bp long (e.g. about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp or more.

In some embodiments, nucleic acids may include a fluorescent signal or sequence tag information. Quantification of the signal or tag may be used in a variety of techniques such as, for example, flow cytometry, quantitative polymerase chain reaction (qPCR), gel electrophoresis, gene-chip analysis, microarray, mass spectrometry, cytofluorimetric analysis, fluorescence microscopy, confocal laser scanning microscopy, laser scanning cytometry, affinity chromatography, manual batch mode separation, electric field suspension, sequencing, and combination thereof.

### Adaptors

In some embodiments, nucleic acids (e.g., PCR primers, PCR amplicons, sample nucleic acid) may include an adaptor sequence and/or complement thereof. Adaptor sequences often are useful for certain sequencing methods such as, for example, a sequencing-by-synthesis process described herein. Adaptors sometimes are referred to as sequencing adaptors or adaptor oligonucleotides. Adaptor sequences typically include one or more sites useful for attachment to a solid support (e.g., flow cell). Adaptors also may include sequencing primer hybridization sites (i.e. sequences complementary to primers used in a sequencing reaction) and identifiers (e.g., indices) as described below. Adaptor sequences can be located at the 5′ and/or 3′ end of a nucleic acid and sometimes can be located within a larger nucleic acid sequence. Adaptors can be any length and any sequence, and may be selected based on standard methods in the art for adaptor design.

One or more adaptor oligonucleotides may be incorporated into a nucleic acid (e.g., PCR amplicon) by any method suitable for incorporating adaptor sequences into a nucleic acid. For example, PCR primers used for generating PCR amplicons (i.e., amplification products) may comprise adaptor sequences or complements thereof. Thus, PCR amplicons that comprise one or more adaptor sequences can be generated during an amplification process. In some cases, one or more adaptor sequences can be ligated to a nucleic acid (e.g., PCR amplicon) by any ligation method suitable for attaching adaptor sequences to a nucleic acid. Ligation processes may include, for example, blunt-end ligations, ligations that exploit 3′ adenine (A) overhangs generated by Taq polymerase during an amplification process and ligate adaptors having 3′ thymine (T) overhangs, and other “sticky-end” ligations. Ligation processes can be optimized such that adaptor sequences hybridize to each end of a nucleic acid and not to each other.

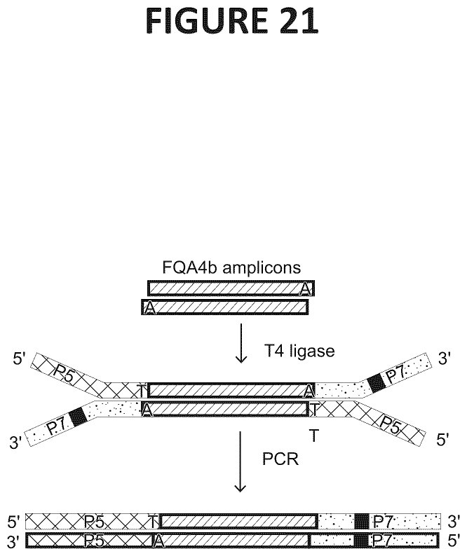

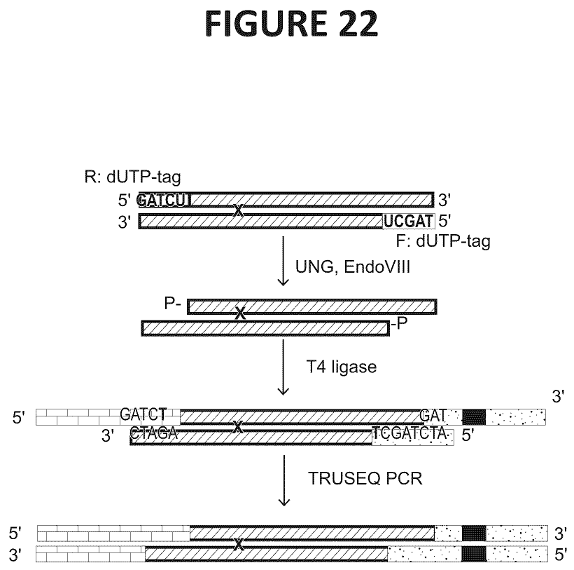

In some cases, adaptor ligation is bidirectional, which means that adaptor sequences are attached to a nucleic acid such that both ends of the nucleic acid are sequenced in a subsequent sequencing process. In some cases, adaptor ligation is unidirectional, which means that adaptor sequences are attached to a nucleic acid such that one end of the nucleic acid is sequenced in a subsequent sequencing process. Examples of unidirectional and bidirectional ligation schemes are discussed in Example 4 and shown in FIGS. 21 and 22.

### Identifiers

In some embodiments, nucleic acids (e.g., PCR primers, PCR amplicons, sample nucleic acid, sequencing adaptors) may include an identifier. In some cases, an identifier is located within or adjacent to an adaptor sequence. An identifier can be any feature that can identify a particular origin or aspect of a genomic target sequence. For example, an identifier (e.g., a sample identifier) can identify the sample from which a particular genomic target sequence originated. In another example, an identifier (e.g., a sample aliquot identifier) can identify the sample aliquot from which a particular genomic target sequence originated. In another example, an identifier (e.g., chromosome identifier) can identify the chromosome from which a particular genomic target sequence originated. An identifier may be referred to herein as a tag, index, barcode, identification tag, index primer, and the like. An identifier may be a unique sequence of nucleotides (e.g., sequence-based identifiers), a detectable label such as the labels described below (e.g., identifier labels), and/or a particular length of polynucleotide (e.g., length-based identifiers; size-based identifiers) such as a stuffer sequence. Identifiers for a collection of samples or plurality of chromosomes, for example, may each comprise a unique sequence of nucleotides. Identifiers (e.g., sequence-based identifiers, length-based identifiers) may be of any length suitable to distinguish certain target genomic sequences from other target genomic sequences. In some embodiments, identifiers may be from about one to about 100 nucleotides in length. For example, identifiers independently may be about 1, 2, 3, 4, 5, 6, 7, 8, 9, 10, 20, 30, 40, 50, 60, 70, 80, 90 or 100 nucleotides in length. In some embodiments, an identifier contains a sequence of six nucleotides. In some cases, an identifier is part of an adaptor sequence for a sequencing process, such as, for example, a sequencing-by-synthesis process described in further detail herein. In some cases, an identifier may be a repeated sequence of a single nucleotide (e.g., poly-A, poly-T, poly-G, poly-C). Such identifiers may be detected and distinguished from each other, for example, using nanopore technology, as described herein.

In some embodiments, the analysis includes analyzing (e.g., detecting, counting, processing counts for, and the like) the identifier. In some embodiments, the detection process includes detecting the identifier and sometimes not detecting other features (e.g., sequences) of a nucleic acid. In some embodiments, the counting process includes counting each identifier. In some embodiments, the identifier is the only feature of a nucleic acid that is detected, analyzed and/or counted.

### Detection of Fetal Aneuploidy

For the detection of fetal aneuploidies, some methods rely on measuring the ratio between maternally and paternally inherited alleles. However, the ability to quantify chromosomal changes is impaired by the maternal contribution of cell free nucleic acids, which makes it necessary to deplete the sample from maternal DNA prior to measurement. Promising approaches take advantage of the different size distribution of fetal and maternal DNA or measure RNA that is exclusively expressed by the fetus (see for example, U.S. patent application Ser. No. 11/384,128, which published as US20060252071 and is hereby incorporated by reference). Assuming fetal DNA makes up only about 5% of all cell free DNA in the maternal plasma, there is a decrease of the ratio difference from 1.6% to only about 1.2% between a trisomy sample and a healthy control. Consequently, reliable detection of allele ratio changes requires enriching the fetal fraction of cell free DNA, for example, using the compositions and methods of the present technology.

Some methods rely on measuring the ratio of maternal to paternally inherited alleles to detect fetal chromosomal aneuploidies from maternal plasma. A diploid set yields a 1:1 ratio while trisomies can be detected as a 2:1 ratio. Detection of this difference is impaired by statistical sampling due to the low abundance of fetal DNA, presence of excess maternal DNA in the plasma sample and variability of the measurement technique. The latter is addressed by using methods with high measurement precision, like digital PCR or mass spectrometry. Enriching the fetal fraction of cell free DNA in a sample is currently achieved by either depleting maternal DNA through size exclusion or focusing on fetal-specific nucleic acids, like fetal-expressed RNA. Another differentiating feature of fetal DNA is its DNA methylation pattern. Thus, provided herein are novel compositions and methods for accurately quantifying fetal nucleic acid based on differential methylation between a fetus and mother. The methods rely on sensitive absolute copy number analysis to quantify the fetal nucleic acid portion of a maternal sample, thereby allowing for the prenatal detection of fetal traits. The methods of the technology herein have identified approximately 3000 CpG rich regions in the genome that are differentially methylated between maternal and fetal DNA. The selected regions showed highly conserved differential methylation across all measured samples. In addition the set of regions is enriched for genes important in developmental regulation, indicating that epigenetic regulation of these areas is a biologically relevant and consistent process (see Table 3). Enrichment of fetal DNA can now be achieved by using the MBD-FC protein to capture all cell free DNA and then elute the highly methylated DNA fraction with high salt concentrations. Using the low salt eluate fractions, the MBD-FC is equally capable of enriching non-methylated fetal DNA.

The present technology provides 63 confirmed genomic regions on chromosomes 13, 18 and 21 with low maternal and high fetal methylation levels. After capturing these regions, SNPs can be used to determine the aforementioned allele ratios. When high frequency SNPs are used around 10 markers have to be measured to achieve a high confidence of finding at least one SNP where the parents have opposite homozygote genotypes and the child has a heterozygote genotype.

In an embodiment, a method for chromosomal abnormality detection is provided that utilizes absolute copy number quantification. A diploid chromosome set will show the same number of copies for differentially methylated regions across all chromosomes, but, for example, a trisomy 21 sample would show 1.5 times more copies for differentially methylated regions on chromosome 21.

Normalization of the genomic DNA amounts for a diploid chromosome set can be achieved by using unaltered autosomes as reference (also provided herein—see Table 1B). Comparable to other approaches, a single marker is less likely to be sufficient for detection of this difference, because the overall copy numbers are low. Typically there are approximately 100 to 200 copies of fetal DNA from 1 ml of maternal plasma at 10 to 12 weeks of gestation. However, the methods of the present technology offer a redundancy of detectable markers that enables highly reliable discrimination of diploid versus aneuploid chromosome sets.

### Data Processing and Identifying Presence or Absence of a Chromosome Abnormality

The term “detection” of a chromosome abnormality as used herein refers to identification of an imbalance of chromosomes by processing data arising from detecting sets of amplified nucleic acid species, nucleotide sequence species, or a detectable product generated from the foregoing (collectively “detectable product”). Any suitable detection device and method can be used to distinguish one or more sets of detectable products, as addressed herein. An outcome pertaining to the presence or absence of a chromosome abnormality can be expressed in any suitable form, including, without limitation, probability (e.g., odds ratio, p-value), likelihood, percentage, value over a threshold, or risk factor, associated with the presence of a chromosome abnormality for a subject or sample. An outcome may be provided with one or more of sensitivity, specificity, standard deviation, coefficient of variation (CV) and/or confidence level, or combinations of the foregoing, in certain embodiments.

Detection of a chromosome abnormality based on one or more sets of detectable products may be identified based on one or more calculated variables, including, but not limited to, sensitivity, specificity, standard deviation, coefficient of variation (CV), a threshold, confidence level, score, probability and/or a combination thereof. In some embodiments, (i) the number of sets selected for a diagnostic method, and/or (ii) the particular nucleotide sequence species of each set selected for a diagnostic method, is determined in part or in full according to one or more of such calculated variables.

In certain embodiments, one or more of sensitivity, specificity and/or confidence level are expressed as a percentage. In some embodiments, the percentage, independently for each variable, is greater than about 90% (e.g., about 90, 91, 92, 93, 94, 95, 96, 97, 98 or 99%, or greater than 99% (e.g., about 99.5%, or greater, about 99.9% or greater, about 99.95% or greater, about 99.99% or greater)). Coefficient of variation (CV) in some embodiments is expressed as a percentage, and sometimes the percentage is about 10% or less (e.g., about 10, 9, 8, 7, 6, 5, 4, 3, 2 or 1%, or less than 1% (e.g., about 0.5% or less, about 0.1% or less, about 0.05% or less, about 0.01% or less)). A probability (e.g., that a particular outcome determined by an algorithm is not due to chance) in certain embodiments is expressed as a p-value, and sometimes the p-value is about 0.05 or less (e.g., about 0.05, 0.04, 0.03, 0.02 or 0.01, or less than 0.01 (e.g., about 0.001 or less, about 0.0001 or less, about 0.00001 or less, about 0.000001 or less)).

For example, scoring or a score may refer to calculating the probability that a particular chromosome abnormality is actually present or absent in a subject/sample. The value of a score may be used to determine for example the variation, difference, or ratio of amplified nucleic detectable product that may correspond to the actual chromosome abnormality. For example, calculating a positive score from detectable products can lead to an identification of a chromosome abnormality, which is particularly relevant to analysis of single samples.

In certain embodiments, simulated (or simulation) data can aid data processing for example by training an algorithm or testing an algorithm. Simulated data may for instance involve hypothetical various samples of different concentrations of fetal and maternal nucleic acid in serum, plasma and the like. Simulated data may be based on what might be expected from a real population or may be skewed to test an algorithm and/or to assign a correct classification based on a simulated data set. Simulated data also is referred to herein as “virtual” data. Fetal/maternal contributions within a sample can be simulated as a table or array of numbers (for example, as a list of peaks corresponding to the mass signals of cleavage products of a reference biomolecule or amplified nucleic acid sequence), as a mass spectrum, as a pattern of bands on a gel, or as a representation of any technique that measures mass distribution. Simulations can be performed in most instances by a computer program. One possible step in using a simulated data set is to evaluate the confidence of the identified results, i.e. how well the selected positives/negatives match the sample and whether there are additional variations. A common approach is to calculate the probability value (p-value) which estimates the probability of a random sample having better score than the selected one. As p-value calculations can be prohibitive in certain circumstances, an empirical model may be assessed, in which it is assumed that at least one sample matches a reference sample (with or without resolved variations). Alternatively other distributions such as Poisson distribution can be used to describe the probability distribution.

In certain embodiments, an algorithm can assign a confidence value to the true positives, true negatives, false positives and false negatives calculated. The assignment of a likelihood of the occurrence of a chromosome abnormality can also be based on a certain probability model.

Simulated data often is generated in an in silico process. As used herein, the term “in silico” refers to research and experiments performed using a computer. In silico methods include, but are not limited to, molecular modeling studies, karyotyping, genetic calculations, biomolecular docking experiments, and virtual representations of molecular structures and/or processes, such as molecular interactions.

As used herein, a “data processing routine” refers to a process, that can be embodied in software, that determines the biological significance of acquired data (i.e., the ultimate results of an assay). For example, a data processing routine can determine the amount of each nucleotide sequence species based upon the data collected. A data processing routine also may control an instrument and/or a data collection routine based upon results determined. A data processing routine and a data collection routine often are integrated and provide feedback to operate data acquisition by the instrument, and hence provide assay-based judging methods provided herein.

As used herein, software refers to computer readable program instructions that, when executed by a computer, perform computer operations. Typically, software is provided on a program product containing program instructions recorded on a computer readable medium, including, but not limited to, magnetic media including floppy disks, hard disks, and magnetic tape; and optical media including CD-ROM discs, DVD discs, magneto-optical discs, and other such media on which the program instructions can be recorded.

Different methods of predicting abnormality or normality can produce different types of results. For any given prediction, there are four possible types of outcomes: true positive, true negative, false positive, or false negative. The term “true positive” as used herein refers to a subject correctly diagnosed as having a chromosome abnormality. The term “false positive” as used herein refers to a subject wrongly identified as having a chromosome abnormality. The term “true negative” as used herein refers to a subject correctly identified as not having a chromosome abnormality. The term “false negative” as used herein refers to a subject wrongly identified as not having a chromosome abnormality. Two measures of performance for any given method can be calculated based on the ratios of these occurrences: (i) a sensitivity value, the fraction of predicted positives that are correctly identified as being positives (e.g., the fraction of nucleotide sequence sets correctly identified by level comparison detection/determination as indicative of chromosome abnormality, relative to all nucleotide sequence sets identified as such, correctly or incorrectly), thereby reflecting the accuracy of the results in detecting the chromosome abnormality; and (ii) a specificity value, the fraction of predicted negatives correctly identified as being negative (the fraction of nucleotide sequence sets correctly identified by level comparison detection/determination as indicative of chromosomal normality, relative to all nucleotide sequence sets identified as such, correctly or incorrectly), thereby reflecting accuracy of the results in detecting the chromosome abnormality.

### EXAMPLES

The following examples are provided by way of illustration only and not by way of limitation. Thus, the examples set forth below illustrate certain embodiments and do not limit the technology. Those of skill in the art will readily recognize a variety of non-critical parameters that could be changed or modified to yield essentially the same or similar results.

In Example 1 below, the Applicants used a new fusion protein that captures methylated DNA in combination with CpG Island array to identify genomic regions that are differentially methylated between fetal placenta tissue and maternal blood. A stringent statistical approach was used to only select regions which show little variation between the samples, and hence suggest an underlying biological mechanism. Eighty-five differentially methylated genomic regions predominantly located on chromosomes 13, 18 and 21 were validated. For this validation, a quantitative mass spectrometry based approach was used that interrogated 261 PCR amplicons covering these 85 regions. The results are in very good concordance (95% confirmation), proving the feasibility of the approach.

Next, the Applicants provide an innovative approach for aneuploidy testing, which relies on the measurement of absolute copy numbers rather than allele ratios.

### Example 1

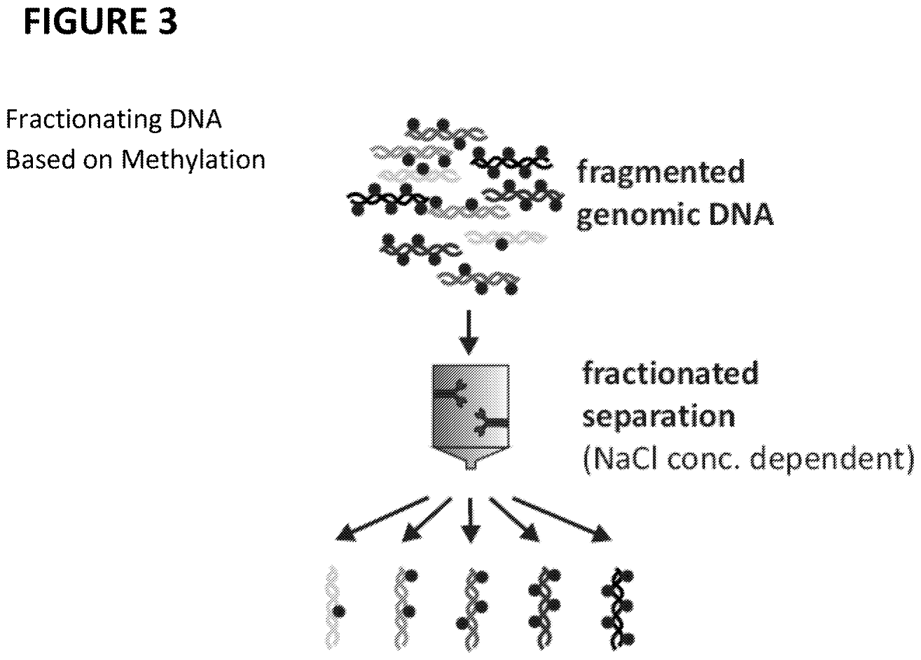

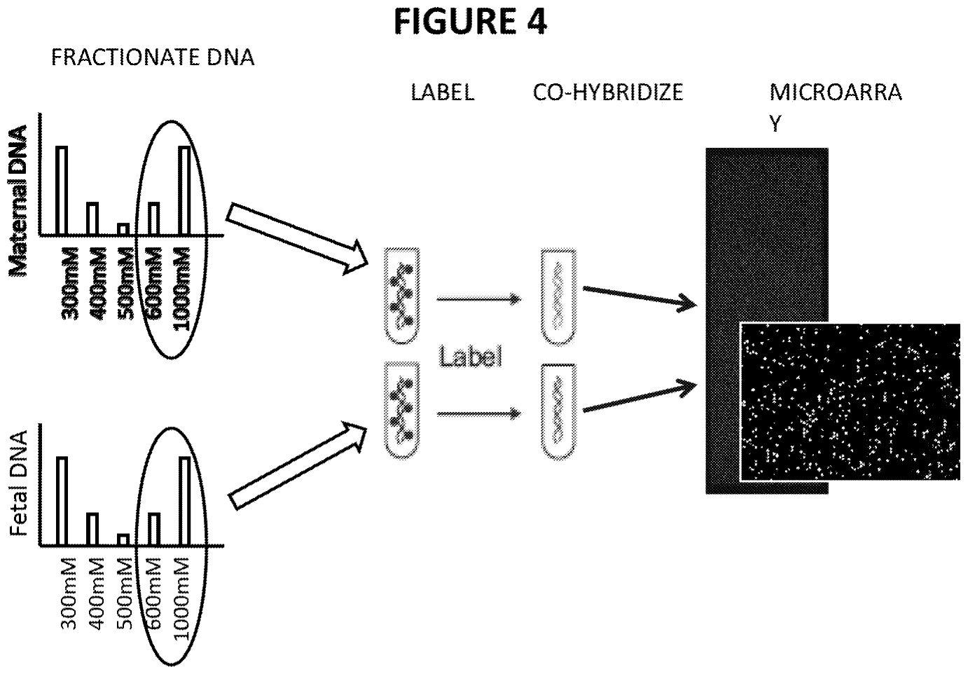

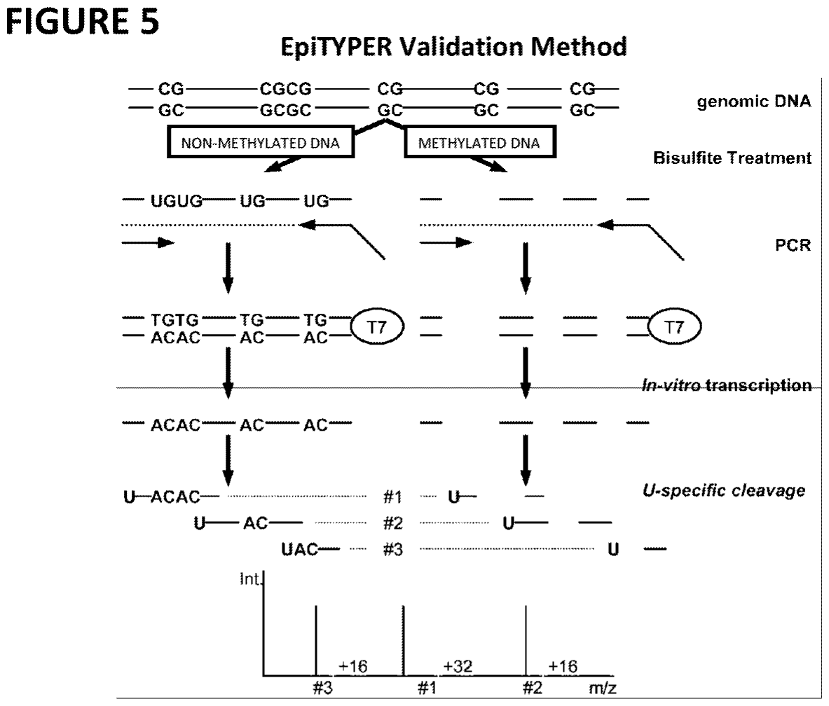

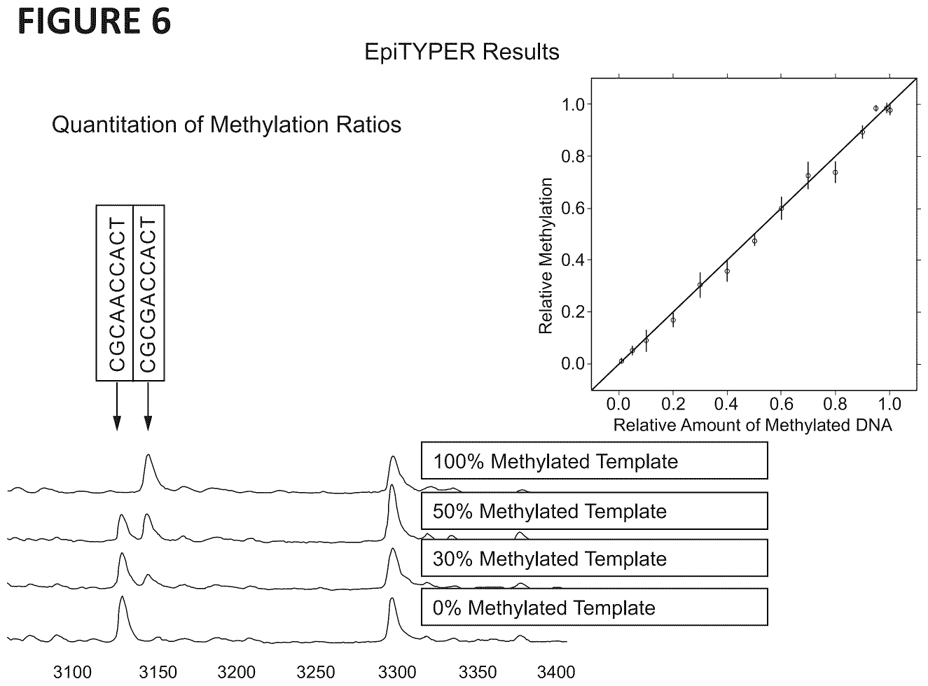

In the below Example, ten paired maternal and placental DNA samples were used to identify differentially methylated regions. These results were validated using a mass spectrometry-based quantitative methylation assay. First, genomic DNA from maternal buffy coat and corresponding placental tissue was first extracted. Next the MBD-FC was used to capture the methylated fraction of each DNA sample. See FIGS. 1-3. The two tissue fractions were labeled with different fluorescent dyes and hybridized to an Agilent® CpG Island microarray. See FIG. 4. This was done to identify differentially methylated regions that could be utilized for prenatal diagnoses. Therefore, two criteria were employed to select genomic regions as potential enrichment markers: the observed methylation difference had to be present in all tested sample pairs, and the region had to be more than 200 bp in length.

### DNA Preparation and Fragmentation

Genomic DNA (gDNA) from maternal buffy coat and placental tissue was prepared using the QIAamp DNA Mini Kit™ and QIAamp DNA Blood Mini Kit™, respectively, from Qiagen® (Hilden, Germany). For MCIp, gDNA was quantified using the NanoDrop ND 1000™ spectrophotometer (Thermo Fisher®, Waltham, MA, USA). Ultrasonication of 2.5 μg DNA in 500 μl TE buffer to a mean fragment size of 300-500 bp was carried out with the Branson Digital Sonifier 450™ (Danbury, CT, USA) using the following settings: amplitude 20%, sonication time 110 seconds, pulse on/pulse off time 1.4/0.6 seconds. Fragment range was monitored using gel electrophoresis.

### Methyl-CpG Immunoprecipitation

Per sample, 56 μg purified MBD-Fc protein and 150 μl of Protein A Sepharose 4 Fast Flow beads (Amersham Biosciences®, Piscataway, NJ, USA) were rotated in 15 ml TBS overnight at 4° C. Then, theMBD-Fc beads (150 μl/assay) were transferred and dispersed in to 2 ml Ultrafree-CL centrifugal filter devices (Millipore®, Billerica, MA, USA) and spin-washed three times with Buffer A (20 mM Tris-HCl, pH8.0, 2 mM MgCl2, 0.5 mM EDTA 300 mM NaCl, 0.1% NP-40). Sonicated DNA (2 μg) was added to the washed MBD-Fc beads in 2 ml Buffer A and rotated for 3 hours at 4° C. Beads were centrifuged to recover unbound DNA fragments (300 mM fraction) and subsequently washed twice with 600 μl of buffers containing increasing NaCl concentrations (400, 500, 550, 600, and 1000 mM). The flow through of each wash step was collected in separate tubes and desalted using a MinElute PCR Purification Kit™ (Qiagen®). In parallel, 200 ng sonicated input DNA was processed as a control using the MinElute PCR Purification Kit™ (Qiagen®).

### Microarray Handling and Analysis

To generate fluorescently labeled DNA for microarray hybridization, the 600 mM and 1M NaCl fractions (enriched methylated DNA) for each sample were combined and labeled with either Alexa Fluor 555-aha-dCTP (maternal) or Alexa Fluor 647-aha-dCTP (placental) using the BioPrime Total Genomic Labeling System™ (Invitrogen®, Carlsbad, CA, USA). The labeling reaction was carried out according to the manufacturer's manual. The differently labeled genomic DNA fragments of matched maternal/placental pairs were combined to a final volume of 80 μl, supplemented with 50 μg Cot-1 DNA (Invitrogen®), 52 μl of Agilent 10× blocking reagent (Agilent Technologies®, Santa Clara, CA, USA), 78 μl of deionized formamide, and 260 μl Agilent 2× hybridization buffer. The samples were heated to 95° C. for 3 min, mixed, and subsequently incubated at 37° C. for 30 min. Hybridization on Agilent CpG Island Microarray Kit™ was then carried out at 67° C. for 40 hours using an Agilent SureHyb™ chamber and an Agilent hybridization oven. Slides were washed in Wash I (6×SSPE, 0.005% N-lauroylsarcosine) at room temperature for 5 min and in Wash II (0.06×SSPE) at 37° C. for an additional 5 min. Next, the slides were submerged in acetonitrile and Agilent Ozone Protection Solution™, respectively, for 30 seconds. Images were scanned immediately and analyzed using an Agilent DNA Microarray Scanner™. Microarray images were processed using Feature Extraction Software v9.5 and the standard CGH protocol.

### Bisulfite Treatment

Genomic DNA sodium bisulfite conversion was performed using EZ-96 DNA Methylation Kit™ (ZymoResearch, Orange County, CA). The manufacturer's protocol was followed using 1 ug of genomic DNA and the alternative conversion protocol (a two temperature DNA denaturation).

### Quantitative Methylation Analysis

Sequenom's MassARRAY® System was used to perform quantitative methylation analysis. This system utilizes matrix-assisted laser desorption ionization time-of-flight (MALDI-TOF) mass spectrometry in combination with RNA base specific cleavage (Sequenom® MassCLEAVE™). A detectable pattern is then analyzed for methylation status. PCR primers were designed using Sequenom® EpiDESIGNER™ (www.epidesigner.com). A total of 261 amplicons, covering 85 target regions, were used for validation (median amplification length=367 bp, min=108, max=500; median number of CpG's per amplicon=23, min=4, max=65). For each reverse primer, an additional T7 promoter tag for in-vivo transcription was added, as well as a 10 mer tag on the forward primer to adjust for melting temperature differences. The MassCLEAVE™ biochemistry was performed as previously described (Ehrich M, et al. (2005) Quantitative high-throughput analysis of DNA methylation patterns by base specific cleavage and mass spectrometry. Proc Natl Acad Sci USA 102:15785-15790). Mass spectra were acquired using a MassARRAY™ Compact MALDI-TOF (Sequenom®, San Diego) and methylation ratios were generated by the EpiTYPER™ software v1.0 (Sequenom®, San Diego).

### Statistical Analysis

All statistical calculations were performed using the R statistical software package (www.r-project.org). First, the array probes were grouped based on their genomic location. Subsequent probes that were less than 1000 bp apart were grouped together. To identify differentially methylated regions, a control sample was used as reference. In the control sample, the methylated fraction of a blood derived control DNA was hybridized against itself. Ideally this sample should show log ratios of the two color channels around 0. However because of the variability in hybridization behavior, the probes show a mean log ratio of 0.02 and a standard deviation of 0.18. Next the log ratios observed in the samples were compared to the control sample. A two way, paired t-test was used to test the NULL hypothesis that the groups are identical. Groups that contained less than 4 probes were excluded from the analysis. For groups including four or five probes, all probes were used in a paired t-test. For Groups with six or more probes, a sliding window test consisting of five probes at a time was used, whereby the window was moved by one probe increments. Each test sample was compared to the control sample and the p-values were recorded. Genomic regions were selected as being differentially methylated if eight out of ten samples showed a p value <0.01, or if six out of ten samples showed a p value <0.001. The genomic regions were classified as being not differentially methylated when the group showed less than eight samples with a p value <0.01 and less than six samples with a p value <0.001. Samples that didn't fall in either category were excluded from the analysis. For a subset of genomic regions that have been identified as differentially methylated, the results were confirmed using quantitative methylation analysis.

The Go analysis was performed using the online GOstat tool (http://gostat.wehi.edu.au/cgibin/-goStat.pl). P values were calculated using Fisher's exact test.

### Microarray-Based Marker Discovery Results

To identify differentially methylated regions a standard sample was used, in which the methylated DNA fraction of monocytes was hybridized against itself. This standard provided a reference for the variability of fluorescent measurements in a genomic region. Differentially methylated regions were then identified by comparing the log ratios of each of the ten placental/maternal samples against this standard. Because the goal of this study was to identify markers that allow the reliable separation of maternal and fetal DNA, the target selection was limited to genes that showed a stable, consistent methylation difference over a contiguous stretch of genomic DNA. This focused the analysis on genomic regions where multiple probes indicated differential methylation. The selection was also limited to target regions where all samples showed differential methylation, excluding those with strong inter-individual differences. Two of the samples showed generally lower log ratios in the microarray analysis. Because a paired test was used for target selection, this did not negatively impact the results.

Based on these selection criteria, 3043 genomic regions were identified that were differentially methylated between maternal and fetal DNA. 21778 regions did not show a methylation difference. No inter-chromosomal bias in the distribution of differentially methylated regions was observed. The differentially methylated regions were located next to or within 2159 known genes. The majority of differentially methylated regions are located in the promoter area (18%) and inside the coding region (68%), while only few regions are located downstream of the gene (7%) or at the transition from promoter to coding region (7%). Regions that showed no differential methylation showed a similar distribution for promoter (13%) and downstream (5%) locations, but the fraction of regions located in the transition of promoter to coding region was higher (39%) and the fraction inside the coding region was lower (43%).

It has been shown in embryonic stem cells (ES) that genes targeted by the polycomb repressive complex2 (PRC2) are enriched for genes regulating development (Lee TI, et al. (2006) Control of developmental regulators by Polycomb in human embryonic stem cells. Cell 125:301-313). It has also been shown that differentially methylated genes are enriched for genes targeted by PRC2 in many cancer types (Ehrich M, et al. (2008) Cytosine methylation profiling of cancer cell lines. Proc Natl Acad Sci USA 105:4844-48). The set of genes identified as differentially methylated in this study is also enriched for genes targeted by PRC2 (p-value <0.001, odds ratio=3.6, 95% Cl for odds ratio=3.1-4.2). A GO analysis of the set of differentially methylated genes reveals that this set is significantly enriched for functions important during development. Six out of the ten most enriched functions include developmental or morphogenic processes [anatomical structure morphogenesis (GO:0009653, p value=0), developmental process (GO:0032502, p value=0), multicellular organismal development (GO:0007275, p value=0), developmental of an organ (GO:0048513, p value=0), system development (GO:0048731, p value=0) and development of an anatomical structure (GO:0048856, p value=0)].

### Validation Using Sequenom® EpiTYPER™

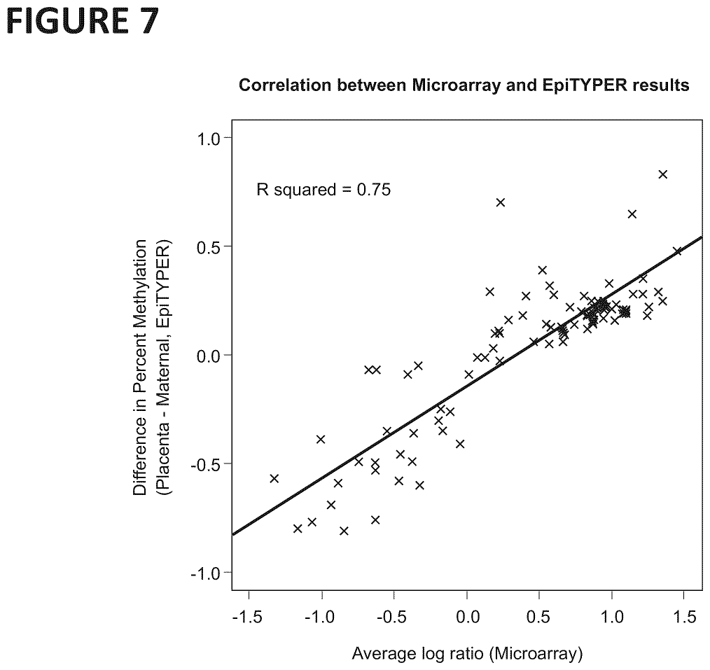

To validate the microarray findings, 63 regions from chromosomes 13, 18 and 21 and an additional 26 regions from other autosomes were selected for confirmation by a different technology. Sequenom EpiTYPER™ technology was used to quantitatively measure DNA methylation in maternal and placental samples. For an explanation of the EpiTYPER™ methods, see Ehrich M, Nelson M R, Stanssens P, Zabeau M, Liloglou T, Xinarianos G, Cantor C R, Field J K, van den Boom D (2005) Quantitative high-throughput analysis of DNA methylation patterns by base specific cleavage and mass spectrometry. Proc Natl Acad Sci USA 102:15785-15790). For each individual CpG site in a target region the average methylation value across all maternal DNA samples and across all placenta samples was calculated. The difference between average maternal and placenta methylation was then compared to the microarray results. The results from the two technologies were in good concordance (see FIG. 7). For 85 target regions the quantitative results confirm the microarray results (95% confirmation rate). For 4 target regions, all located on chromosome 18, the results could not be confirmed. The reason for this discrepancy is currently unclear.

In contrast to microarrays, which focus on identification of methylation differences, the quantitative measurement of DNA methylation allowed analysis of absolute methylation values. In the validation set of 85 confirmed differentially methylated regions, a subset of 26 regions is more methylated in the maternal DNA sample and 59 regions are more methylated in the placental sample (see Table 1A). Interestingly, genes that are hypomethylated in the placental samples tend to show larger methylation differences than genes that are hypermethylated in the placental sample (median methylation difference for hypomethylated genes=39%, for hypermethylated genes=20%).

### Example 2

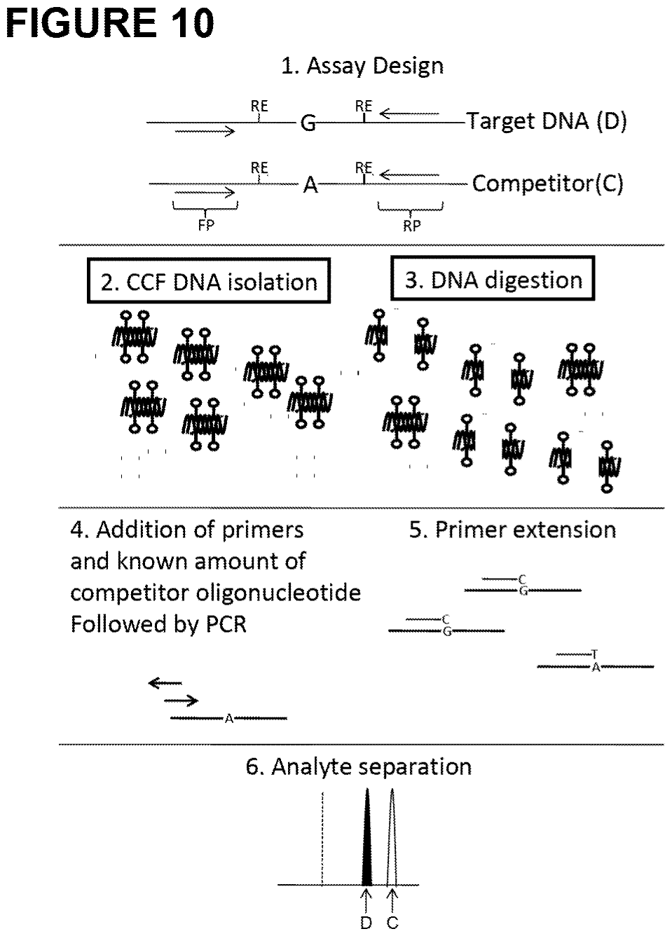

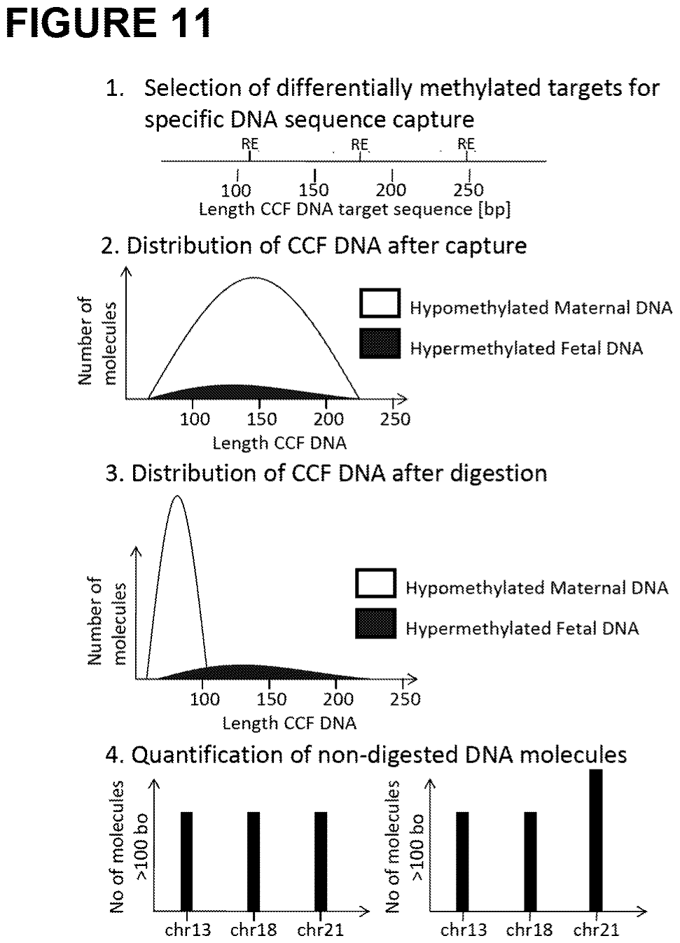

Example 2 describes a non-invasive approach for detecting the amount of fetal nucleic acid present in a maternal sample (herein referred to as the “Fetal Quantifier Method”), which may be used to detect or confirm fetal traits (e.g., fetal sex of RhD compatibility), or diagnose chromosomal abnormalities such as Trisomy 21 (both of which are herein referred to as the “Methylation-Based Fetal Diagnostic Method”). FIG. 10 shows one embodiment of the Fetal Quantifier Method, and FIG. 11 shows one embodiment of the Methylation-Based Fetal Diagnostic Method. Both processes use fetal DNA obtained from a maternal sample. The sample comprises maternal and fetal nucleic acid that is differentially methylated. For example, the sample may be maternal plasma or serum. Fetal DNA comprises approximately 2-30% of the total DNA in maternal plasma. The actual amount of fetal contribution to the total nucleic acid present in a sample varies from pregnancy to pregnancy and can change based on a number of factors, including, but not limited to, gestational age, the mother's health and the fetus' health.

As described herein, the technical challenge posed by analysis of fetal DNA in maternal plasma lies in the need to be able to discriminate the fetal DNA from the co-existing background maternal DNA. The methods of the present technology exploit such differences, for example, the differential methylation that is observed between fetal and maternal DNA, as a means to enrich for the relatively small percentage of fetal DNA present in a sample from the mother. The non-invasive nature of the approach provides a major advantage over conventional methods of prenatal diagnosis such as, amniocentesis, chronic villus sampling and cordocentesis, which are associated with a small but finite risk of fetal loss. Also, because the method is not dependent on fetal cells being in any particular cell phase, the method provides a rapid detection means to determine the presence and also the nature of the chromosomal abnormality. Further, the approach is sex-independent (i.e., does not require the presence of a Y-chromosome) and polymorphic-independent (i.e., an allelic ratio is not determined). Thus, the compositions and methods of the technology herein represent improved universal, noninvasive approaches for accurately determining the amount of fetal nucleic acid present in a maternal sample.

### Assay Design and Advantages

There is a need for accurate detection and quantification of fetal DNA isolated noninvasively from a maternal sample. The present technology takes advantage of the presence of circulating, cell free fetal nucleic acid (ccfDNA) in maternal plasma or serum. In order to be commercially and clinically practical, the methods of the technology herein should only consume a small portion of the limited available fetal DNA. For example, less than 50%, 40%, 30%, 25%, 20%, 15%, 10%, 5% or less of the sample. Further, the approach should preferably be developed in a multiplex assay format in which one or more (preferably all) of the following assays are included:
Assays for the detection of total amount of genomic equivalents present in the sample, i.e., assays recognizing both maternal and fetal DNA species;
Assays for the detection of fetal DNA isolated from a male pregnancy, i.e., sequences specific for chromosome Y;
Assays specific for regions identified as differentially methylated between the fetus and mother; or
Assays specific for regions known to be hypomethylated in all tissues to be investigated, which can serve as a control for restriction efficiency.

Other features of the assay may include one or more of the following:
For each assay, a target-specific, competitor oligonucleotide that is identical, or substantially identical, to the target sequence apart from a distinguishable feature of the competitor, such as a difference in one or more nucleotides relative to the target sequence. This oligonucleotide when added into the PCR reaction will be co-amplified with the target and a ratio obtained between these two PCR amplicons will indicate the number of target specific DNA sequences (e.g., fetal DNA from a specific locus) present in the maternal sample.
The amplicon lengths should preferably be of similar length in order not to skew the amplification towards the shorter fragments. However, as long as the amplification efficiency is about equal, different lengths may be used.
Differentially methylated targets can be selected from Tables 1A-1C or from any other targets known to be differentially methylated between mother and fetus. These targets can be hypomethylated in DNA isolated from non-pregnant women and hypermethylated in samples obtained from fetal samples. These assays will serve as controls for the restriction efficiency.
The results obtained from the different assays can be used to quantify one or more of the following:
   Total number of amplifiable genomes present in the sample (total amount of genomic equivalents);
   The fetal fraction of the amplifiable genomes (fetal concentration or percentage); or
   Differences in copy number between fetally-derived DNA sequences (for example, between fetal chromosome 21 and a reference chromosome such as chromosome 3).

### Examples of Assays Used in the Test

Below is an outline of the reaction steps used to perform a method of the technology herein, for example, as provided in FIG. 10. This outline is not intended to limit the scope of the technology herein. Rather it provides one embodiment of the technology herein using the Sequenom® MassARRAY® technology.
1) DNA isolation from plasma samples.
2) Digestion of the DNA targets using methylation sensitive restriction enzymes (for example, HhaI and HpaII).

For each reaction the available DNA was mixed with water to a final volume of 25 ul.

10 ul of a reaction mix consisting of 10 units HhaI, 10 units HpaII and a reaction buffer were added. The sample was incubated at an optimal temperature for the restriction enzymes. HhaI and HpaII digest non-methylated DNA (and will not digest hemi- or completely methylated DNA). Following digestion, the enzymes were denatured using a heating step.
3) Genomic Amplification—PCR was performed in a total volume of 50 ul by adding PCR reagents (Buffer, dNTPs, primers and polymerase). Exemplary PCR and extend primers are provided below. In addition, synthetic competitor oligonucleotide was added at known concentrations.
4) Replicates (optional)—Following PCR the 50 ul reaction was split into 5 ul parallel reactions (replicates) in order to minimize variation introduced during the post PCR steps of the test. Post PCR steps include SAP, primer extension (MassEXTEND® technology), resin treatment, dispensing of spectrochip and MassARRAY.
5) Quantification of the Amplifiable Genomes—Sequenom MassARRAY® technology was used to determine the amount of amplification product for each assay. Following PCR, a single base extension assay was used to interrogate the amplified regions (including the competitor oligonucleotides introduced in step 3). Specific extend primers designed to hybridize directly adjacent to the site of interest were introduced. See extend primers provided below. These DNA oligonucleotides are referred to as iPLEX® MassEXTEND® primers. In the extension reaction, the iPLEX primers were hybridized to the complementary DNA templates and extended with a DNA polymerase. Special termination mixtures that contain different combinations of deoxy- and dideoxynucleotide triphosphates along with enzyme and buffer, directed limited extension of the iPLEX primers. Primer extension occurs until a complementary dideoxynucleotide is incorporated.

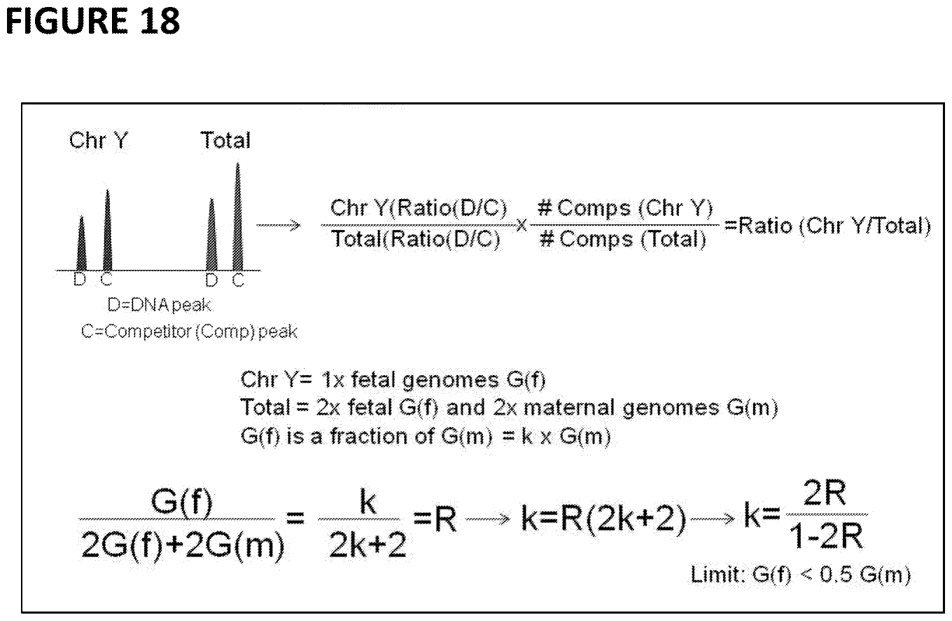

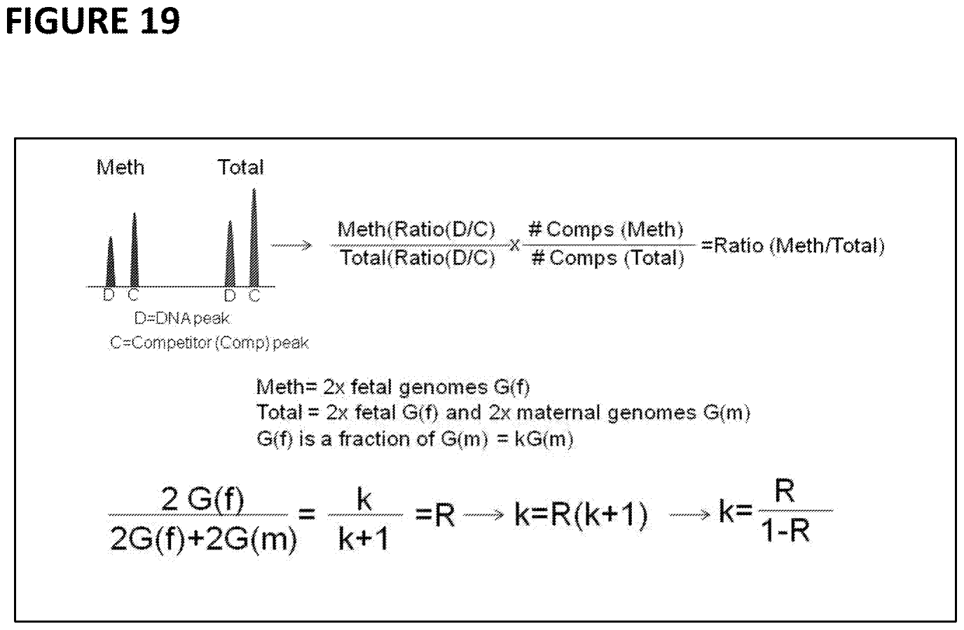

The extension reaction generated primer products of varying length, each with a unique molecular weight. As a result, the primer extension products can be simultaneously separated and detected using Matrix Assisted Laser Desorption/Ionization, Time-Of-Flight (MALDI-TOF) mass spectrometry on the MassARRAY® Analyzer Compact. Following this separation and detection, SEQUENOM's proprietary software automatically analyzes the data.
6) Calculating the amount and concentration of fetal nucleic acid—Methods for calculating the total amount of genomic equivalents present in the sample, the amount (and concentration) of fetal nucleic acid isolated from a male pregnancy, and the amount (and concentration) of fetal nucleic based on differentially methylated targets are provided below and in FIGS. 18 and 19.

The above protocol can be used to perform one or more of the assays described below. In addition to the sequences provided immediately below, a multiplex scheme that interrogates multiple targets is provided in Table X below.

### 1) Assay for the Quantification of the Total Number of Amplifiable Genomic Equivalents in the Sample.

Targets were selected in housekeeping genes not located on the chromosomes 13, 18, 21, X or Y. The targets should be in a single copy gene and not contain any recognition sites for the methylation sensitive restriction enzymes.

Underlined sequences are PCR primer sites, italic is the site for the single base extend primer and bold letter (C) is the nucleotide extended on human DNA
ApoE Chromosome 19:45409835-45409922 DNA target sequence with interrogated nucleotide C in bold. All of the chromosome positions provided in this section are from the February 2009 UCSC Genome Build.

(SEQ ID NO: 262)
GATTGACAGTTTCTCCTTCCCCAGACTGGCCAATCACAGGCAGGAAGATG
AAGGTTCTGTGGGCTGCGTTGCTGGTCACATTCCTGGC
ApoE Forward Primer:
(SEQ ID NO: 263)
5′-ACGTTGGATG-TTGACAGTTTCTCCTTCCCC
(Primer contains a 5′ 10 bp MassTag separated by
a dash)
ApoE Reverse Primer:
(SEQ ID NO: 264)
5′-ACGTTGGATG-GAATGTGACCAGCAACGCAG
(Primer contains a 5′ 10 bp MassTag separated by
a dash)
ApoE Extension Primer:
(SEQ ID NO: 265)
5′-GCAGGAAGATGAAGGTT [C/T]
Primer extends C on human DNA targets and T on
synthetic DNA targets
ApoE synthetic competitor oligonucleotide:
(SEQ ID NO: 266)
5′-GATTGACAGTTTCTCCTTCCCCAGACTGGCCAATCACAGGCAGGAAG
ATGAAGGTTTTGTGGGCTGCGTTGCTGGTCACATTCCTGGC
(Bold T at position 57 is different from human
DNA)

### 2) Assay for the Quantification of the Total Number of Chromosome Y Sequences in the Sample.

Targets specific for the Y-chromosome were selected, with no similar or paralog sequences elsewhere in the genome. The targets should preferably be in a single copy gene and not contain any recognition sites for the methylation sensitive restriction enzyme(s).

Underlined sequences are PCR primer sites, and italic nucleotide(s) is the site for the single-base extend primer and bold letter (C) is the nucleotide extended on human DNA.

SRY on chrY:2655628-2655717 (reverse complement)
(SEQ ID NO: 267)
GAGTTTTGGATAGTAAAATAAGTTTCGAACTCTGGCACCTTTCAATTTTG
TCGCACTCTCCTTGTTTTTGACAATGCAATCATATGCTTC
SRY Forward Primer:
(SEQ ID NO: 268)
5′-ACG-TGGATAGTAAAATAAGTTTCGAACTCTG
(Primer contains a 5′ 3 bp MassTag separated by
a dash)
SRY Reverse Primer:
(SEQ ID NO: 269)
5′-GAAGCATATGATTGCATTGTCAAAAAC
SRY Extension Primer:
(SEQ ID NO: 270)
5′-aTTTCAATTTTGTCGCACT [C/T]
Primer extends C on human DNA targets and T on
synthetic DNA targets. 5′ Lower case “a” is a
non-complementary nucleotide
SRY synthetic competitor oligonucleotide:
(SEQ ID NO: 271)
5′-GAGTTTTGGATAGTAAAATAAGTTTCGAACTCTGGCACCTTTCAATT
TTGTCGCACTTTCCTTGTTTTTGACAATGCAATCATATGCTTC

### 3) Assay for the Quantification of Fetal Methylated DNA Sequences Present in the Sample.

Targets were selected in regions known to be differentially methylated between maternal and fetal DNA. Sequences were selected to contain several restriction sites for methylation sensitive enzymes. For this study the HhaI (GCGC) and HpaII (CCGG) enzymes were used.

Underlined sequences are PCR primer sites, italic is the site for the single base extend primer and bold letter (C) is the nucleotide extended on human DNA, lower case letter are recognition sites for the methylation sensitive restriction enzymes.

TBX3on chr12:115124905-115125001
(SEQ ID NO: 272)
GAACTCCTCTTTGTCTCTGCGTGCccggcgcgcCCCCCTCccggTGGGTG
ATAAACCCACTCTGgcgccggCCATgcgcTGGGTGATTAATTTGCGA
TBX3 Forward Primer:
(SEQ ID NO: 273)
5′-ACGTTGGATG-TCTTTGTCTCTGCGTGCCC
(Primer contains a 5′ 10 bp MassTag separated by
a dash)
TBX3 Reverse Primer:
(SEQ ID NO: 274)
5′-ACGTTGGATG-TTAATCACCCAGCGCATGGC
(Primer contains a 5′ 10 bp MassTag separated by
a dash)
TBX3 Extension Primer:
(SEQ ID NO: 275)
5′-CCCCTCCCGGTGGGTGATAAA [C/T]
Primer extends C on human DNA targets and T on
synthetic DNA targets. 5′ Lower case “a” is a
non-complementary nucleotide
TBX3 synthetic competitor oligonucleotide:
(SEQ ID NO: 276)
5′-GAACTCCTCTTTGTCTCTGCGTGCCCGGCGCGCCCCCCTCCCGGTGG
GTGATAAATCCACTCTGGCGCCGGCCATGCGCTGGGTGATTAATTTGCGA

### 4) Control Assay for the Enzyme Restriction Efficiency.

Targets were selected in regions known not to be methylated in any tissue to be investigated. Sequences were selected to contain no more than one site for each restriction enzyme to be used.

Underlined sequences are PCR primer sites, italic nucleotide(s) represent the site for the single-base extend primer and bold letter (G) is the reverse nucleotide extended on human DNA, lower case letter are recognition sites for the methylation sensitive restriction enzymes.

CACNA1G chr17:48637892-48637977 (reverse
complement)
(SEQ ID NO: 277)
CCATTGGCCGTCCGCCGTGGCAGTGCGGGCGGGAgcgcAGGGAGAGAACC
ACAGCTGGAATCCGATTCCCACCCCAAAACCCAGGA
Hhal Forward Primer:
(SEQ ID NO: 278)
5′-ACGTTGGATG-CCATTGGCCGTCCGCCGTG
(Primer contains a 5′ 10 bp MassTag separated by
a dash)
Hhal Reverse Primer:
(SEQ ID NO: 279)
5′-ACGTTGGATG-TCCTGGGTTTTGGGGTGGGAA
(Primer contains a 5′ 10 bp MassTag separated by
a dash)
Hhal Extension Primer:
(SEQ ID NO: 280)
5′-TTCCAGCTGTGGTTCTCTC
Hhal synthetic competitor oligonucleotide:
(SEQ ID NO: 281)
5′-CCATTGGCCGTCCGCCGTGGCAGTGCGGGCGGGAGCGCAGAGAGAGA
ACCACAGCTGGAATCCGATTCCCACCCCAAAACCCAGGA

### Validation Experiments

The sensitivity and accuracy of the present technology was measured using both a model system and clinical samples. In the different samples, a multiplex assay was run that contains 2 assays for total copy number quantification, 3 assays for methylation quantification, 1 assay specific for chromosome Y and 1 digestion control assay. See Table X. Another multiplex scheme with additional assays is provided in Table Y.

TABLE X
PCR Primers and Extend Primers

| | | First Primer | Second Primer | Extend Primer |
|---|---|---|---|---|
| Gene ID | * | (SEQ ID NOS 282-288) | (SEQ ID NOS 289-295) | (SEQ ID NOS296-302) |
| SOX14 | M | ACGTTGGATGACATGGTCGGCCCCACG | ACGTTGGATGCTCCTTCCTAGTGTGAGAACCG | CAGGTTCCGGGGCTTGGG |
| | | GAAT | | |
| Hhal_CTRL | D | ACGTTGGATGACCCATTGGCCGTCCGC | ACGTTGGATGTTTTGGGGTGGGAATCGGATT | CGCAGGGAGAGAACCACAG |
| | | CGT | | |
| TBX3 | M | ACGTTGGATGGAACTCCTCTTTGTCTC | ACGTTGGATGTGGCATGGCCGGCGCCAGA | CCCCTCCCGGTGGGTGATAAA |
| | | TGCG | | |
| SRY | Y | ACGTTGGATGCGCAGCAACGGGACCGC | ACGTTGGCATCTAGGTAGGTCTTTGTAGCCAA | AAAGCTGTAGGACAATCGGGT |
| | | TACA | | |
| ALB | T | ACGTTGCGTAGCAACCTGTTACATATT | ACGTTGGATCTGAGCAAAGGCAATCAACACCC | CATTTTTCTACATCCTTTGTTT |
| | | AA | | |
| EDG6 | M | ACGTTGGATGCATAGAGGCCCATGATG | ACGTTGGATGACCTTCTGCCCCTCTACTCCAA | agAAGATCACCAGGCAGAAGAG |
| | | GTGG | | G |
| RNaseP | T | ACGTTGGATGGTGTGGTCAGCTCTTCC | ACGTTGGCCCACATGTAATGTGTTGAAAAAG | ACTTGGAGAACAAAGGACACCG |
| | | CTTCAT | CA | TTA |

TABLE X
Competitor Oligonucleotide Sequence

| Gene ID | * | Competitor Oligonucleotide Sequence (SEQ ID NOS 303-309) |
|---|---|---|
| SOX14 | M | GGTCGGCCCCACGGAATCCCGGCTCTGTGTGCGCCCAGGTTCCGGGGCTTGGGTGTTGCCGGTTCTCACACTAGGAAGGAG |
| Hhal_CTRL | D | CCATTGGCCGTCCGCCGTGGCAGTGCGGGCGGGAGCGCAGAGAGAGAACCACAGCTGGAATCCGATTCCCACCCCAAAA |
| TBX3 | M | GAACTCCTCTTTGTCTCTGCGTGCCCGGCGCGCCCCCCTCCCGGTGGGTGATAAATCCACTCTGGCGCCGGCCATGC |
| SRY | Y | GCAGCAACGGGACCGCTACAGCCACTGGACAAAGCCGTAGGACAATCGGGTAACATTGGCTACAAAGACCTACCTAGATGC |
| ALB | T | GCGTAGCAACCTGTTACATATTAAAGTTTTATTATACTACATTTTTCTACATCCTTTGTTTCAGAGTGTTGATTGCCTTTGCTCAG |
| | | TATCTTCAG |
| EDG6 | M | CCTTCTGCCCCTCTACTCCAAGCGCTACACCCTCTTCTGCCTGGTGATCTTTGCCGGCGTCCTGGCCACCATCATGGGCCTCTATG |
| RNaseP | T | GTGTGGTCAGCTCTTCCCTTCATCACATACTTGGAGAACAAAGGACACCGTTATCCATGCTTTTTCAACACATTACATGTGGG |

TABLE Y
PCR Primers and Extend Primers

| | | First Primer | Second Primer | Extend Primer |
|---|---|---|---|---|
| Gene ID | * | (SEQ ID NOS 310-319) | (SEQ ID NOS 320-329) | (SEQ ID NOS 330-339) |
| EDG6 | M | ACGTTGGATGTTCTGCCCCTCTACTCCAAG | ACGTTGGATGCATAGAGGCCCATGATGGTG | TTCTGCCTGGTGATCTT |
| RNAseP | T | ACGTTGGATGTCAGCTCTTCCCTTCATCAC | ACGTTGGATGCCTACCTCCCACATGTAATGT | AACAAAGGACACCGTTA |
| ApoE | T | ACGTTGGATGTTGACAGTTTCTCCTTCCCC | ACGTTGGATGGAATGTGACCAGCAACGCAG | GCAGGAAGATGAAGGTT |
| SOX14 | M | ACGTTGGATGCGGTCGGCCCCACGGAAT | ACGTTGGATGCTCCTTCCTAGTGTGAGAACCG | aAGGTTCCGGGGCTTGGG |
| SRY no2 | Y | ACGTGGATAGTAAAATAAGTTTCGAACTCTG | GAAGCATATGATTGCATTGTCAAAAAC | aTTTCAATTTTGTCGCACT |
| SRY no1 | Y | ACGTTGGATGCACAGCTCACCGCAGCAACG | ACGTTGGATGCTAGGTAGGTCTTTGTAGCCAA | AGCTGTAGGACAATCGGGT |
| TBX3 | M | ACGTTGGATGTCTTTGTCTCTGCGTGCCC | ACGTTGGATGTTAATCACCCAGCGCATGGC | CCCTCCCGGTGGGTGATAAA |
| CACNA1G | D | ACGTTGGATGGACTGAGCCCCAGAACTCG | ACGTTGGATGGTGGGTTTGTGCTTTCCACG | AGGGCCGGGGTCTGCGCGTG |
| dig CTRL 1 | | | | |
| DAPK1 dig | D | ACGTTGGATGAAGCCAAGTTTCCCTCCGC | ACGTTGGATGCTTTTGCTTTCCCAGCCAGG | GAGGCACTGCCCGGACAAACC |
| CTRL 2 | | | | |
| ALB | T | ACGTTAGCGTAGCAACCTGTTACATATTAA | ACGTTGGATGCTGAGCAAAGGCAATCAACA | CATTTTTCTACATCCTTTGTTT |

TABLE Y
Competitor Oligonucleotide Sequence

| Gene ID | * | Competitor (SEQ ID NOS 340-349) |
|---|---|---|
| EDG6 | M | CCTTCTGCCCCTCTACTCCAAGCGCTACACCCTCTTCTGCCTGGTGATCTTTGCCGGCGTCCTGGCCACCATCATGGGCCTCTATG |
| RNAseP | T | GTGTGGTCAGCTCTTCCCTTCATCACATACTTGGAGAACAAAGGACACCGTTATCCATGCTTTTTCAACACATTACATGTGGGAGG |
| | | TAGG |
| ApoE | T | GATTGACAGTTTCTCCTTCCCCAGACTGGCCAATCACAGGCAGGAAGATGAAGGTTTTGTGGGCTGCGTTGCTGGTCACATTCCTG |
| | | GC |
| SOX14 | M | AAAACCAGAGATTCGCGGTCGGCCCCACGGAATCCCGGCTCTGTGTGCGCCCAGGTTCCGGGGCTTGGGTGTTGCCGGTTCTCACA |
| | | CTAGGAAGGAGC |
| SRY no2 | Y | GAGTTTTGGATAGTAAAATAAGTTTCGAACTCTGGCACCTTTCAATTTTGTCGCACTTTCCTTGTTTTTGACAATGCAATCATATG |
| | | CTTC |
| SRY no1 | Y | GCAGCCAGCTCACCGCAGCAACGGGACCGCTACAGCCACTGGACAAAGCTGTAGGACAATCGGGTGACATTGGCTACAAAGACCTA |
| | | CCTAGATGC |
| TBX3 | M | GAACTCCTCTTTGTCTCTGCGTGCCCGGCGCGCCCCCCTCCCGGTGGGTGATAAATCCACTCTGGCGCCGGCCATGCGCTGGGTGA |
| | | TTAATTTGCG |
| CACNA1G | D | GTGGGTTTGTGCTTTCCACGCGTGCACACACACGCGCAGACCCCGGCCCTTGCCCCGCCTACCTCCCCGAGTTCTGGGGCTCAGTC |
| dig CTRL 1 | | |
| DAPK1 dig | D | GCGCCAGCTTTTGCTTTCCCAGCCAGGGCGCGGTGAGGTTTGTCCGGGCAGTGCCTCGAGCAACTGGGAAGGCCAAGGCGGAGGGA |
| CTRL 2 | | AAC |
| ALB | T | GCGTAGCAACCTGTTACATATTAAAGTTTTATTATACTACATTTTTCTACATCCTTTGTTTTAGGGTGTTGATTGCCTTTGCTCAG |
| | | TATCTTCAGC |

T = Assay for Total Amount
M = Assay for Methylation quantification
Y = Y-Chromosome Specific Assay
D = Digestion control

### Model System Using Genomic DNA

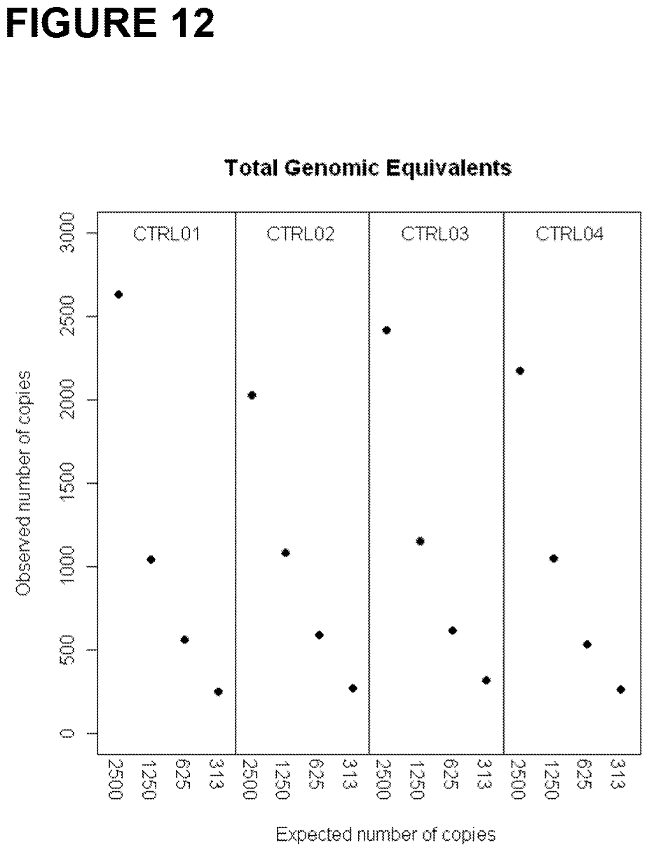

In order to determine the sensitivity and accuracy of the method when determining the total number of amplifiable genomic copies in a sample, a subset of different DNA samples isolated from the blood of non-pregnant women was tested. Each sample was diluted to contain approximately 2500, 1250, 625 or 313 copies per reaction. The total number of amplifiable genomic copies was obtained by taking the mean DNA/competitor ratio obtained from the three total copy number assays. The results from the four different samples are shown in FIG. 12.

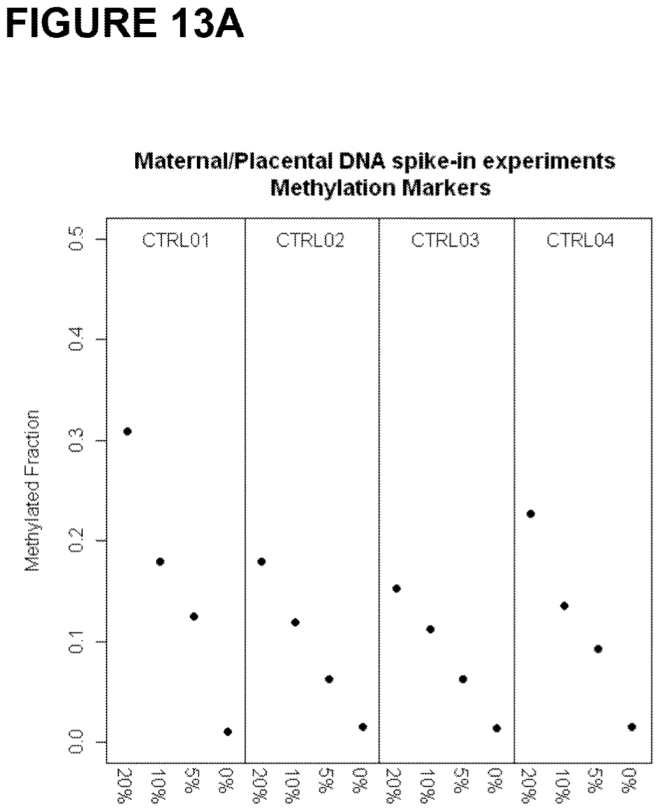

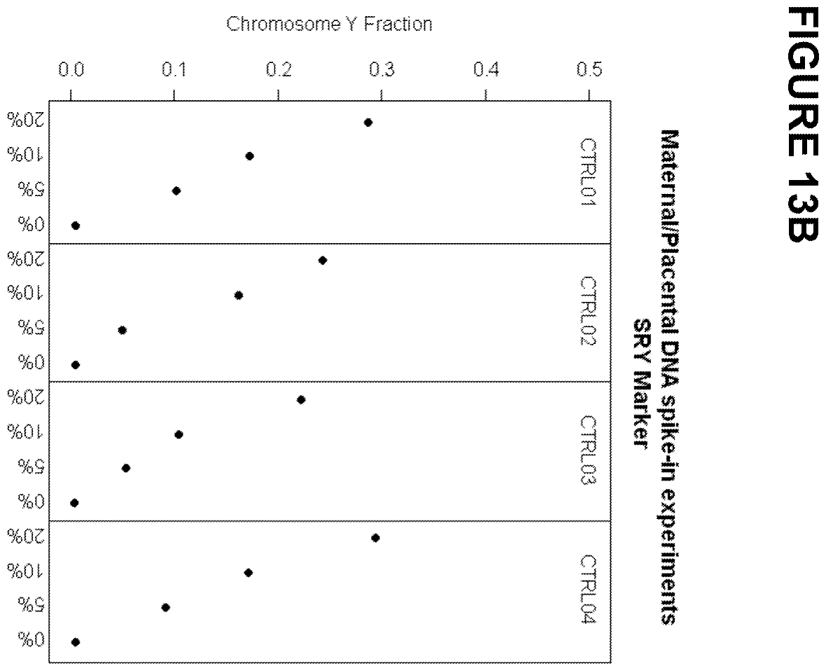

To optimize the reaction, a model system was developed to simulate DNA samples isolated from plasma. These samples contained a constant number of maternal non-methylated DNA and were spiked with different amounts of male placental methylated DNA. The samples were spiked with amounts ranging from approximately 0 to 25% relative to the maternal non-methylated DNA. The results are shown in FIGS. 13A and B. The fraction of placental DNA was calculated using the ratios obtained from the methylation assays (FIG. 13A), the SRY markers (FIG. 13B) and the total copy number assays. The primer sequences for the methylation assays (TBX), Y-chromosome assays (SRY) and total copy number (APOE) are provided above. The model system demonstrated that the methylation-based method performed equal to the Y-chromosome method (SRY markers), thus validating the methylation-based method as a sex-independent fetal quantifier.

### Plasma Samples

To investigate the sensitivity and accuracy of the methods in clinical samples, 33 plasma samples obtained from women pregnant with a male fetus were investigated using the multiplex scheme from Table X. For each reaction, a quarter of the DNA obtained from a 4 ml extraction was used in order to meet the important requirement that only a portion of the total sample is used.

### Total Copy Number Quantification

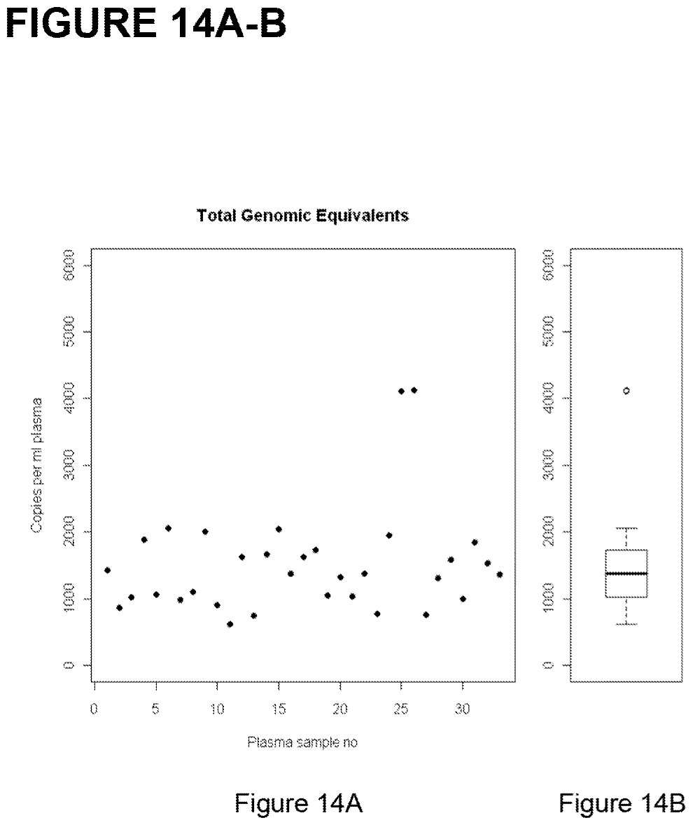

The results from the total copy number quantification can be seen in FIGS. 14A and B. In FIG. 14A, the copy number for each sample is shown. Two samples (nos. 25 and 26) have a significantly higher total copy number than all the other samples. In general, a mean of approximately 1300 amplifiable copies/ml plasma was obtained (range 766-2055). FIG. 14B shows a box-and-whisker plot of the given values, summarizing the results.

Correlation Between Results Obtained from the Methylation Markers and the Y-Chromosome Marker

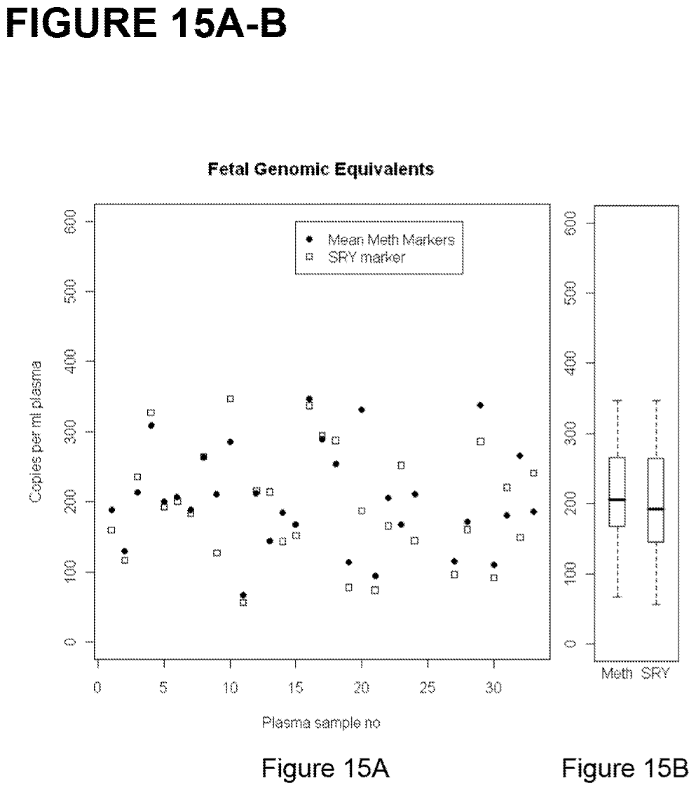

In FIGS. 15A and B, the numbers of fetal copies for each sample are plotted. As all samples were from male pregnancies. The copy numbers obtained can be calculated using either the methylation or the Y-chromosome-specific markers. As can be seen in FIG. 15B, the box-and-whisker plot of the given values indicated minimal difference between the two different measurements.

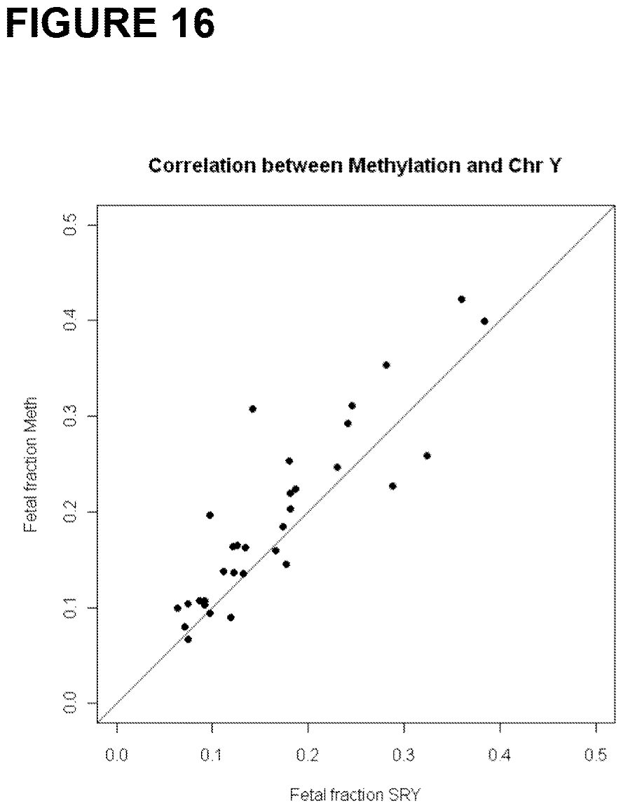

The results showing the correlation between results obtained from the methylation markers and the Y-chromosome marker (SRY) is shown in FIG. 16. Again, the methylation-based method performed equal to the Y-chromosome method (SRY markers), further validating the methylation-based method as a sex-independent and polymorphism-independent fetal quantifier. The multiplexed assays disclosed in Table X were used to determine the amount fetal nucleic.

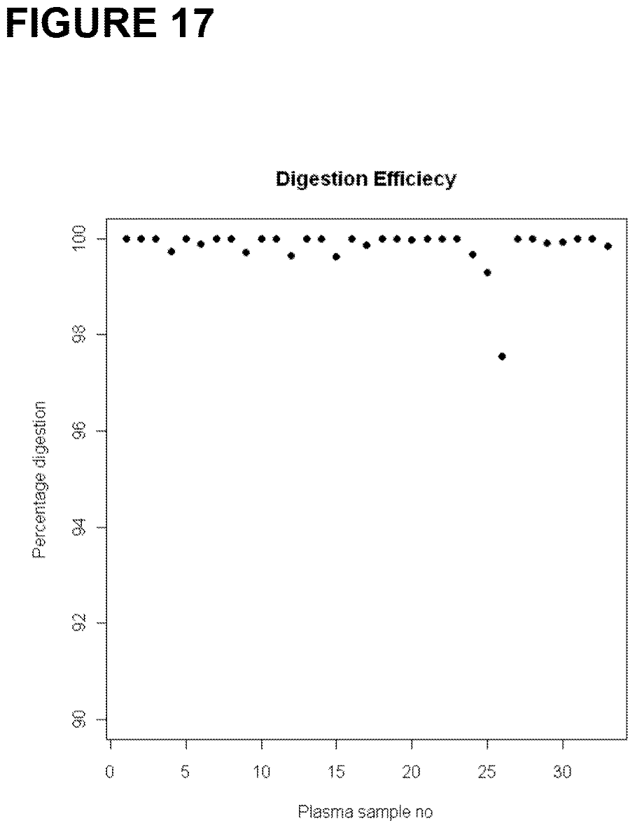

Finally, the digestion efficiency was determined by using the ratio of digestion for the control versus the competitor and comparing this value to the mean total copy number assays. See FIG. 17. Apart from sample 26 all reactions indicate the efficiency to be above 99%.

### Data Analysis

Mass spectra analysis was done using Typer 4 (a Sequenom software product). The peak height (signal over noise) for each individual DNA analyte and competitor assay was determined and exported for further analysis.

The total number of molecules present for each amplicon was calculated by dividing the DNA specific peak by the competitor specific peak to give a ratio. (The “DNA” Peak in FIGS. 18 and 19 can be thought of as the analyte peak for a given assay). Since the number of competitor molecules added into the reaction is known, the total number of DNA molecules can be determined by multiplying the ratio by the number of added competitor molecules.

The fetal DNA fraction (or concentration) in each sample was calculated using the Y-chromosome-specific markers for male pregnancies and the mean of the methylated fraction for all pregnancies. In brief, for chromosome Y, the ratio was obtained by dividing the analyte (DNA) peak by the competitor peak and multiplying this ratio by the number of competitor molecules added into the reaction. This value was divided by a similar ratio obtained from the total number of amplifiable genome equivalents determination (using the Assay(s) for Total Amount). See FIG. 18. Since the total amount of nucleic acid present in a sample is a sum of maternal and fetal nucleic acid, the fetal contribution can be considered to be a fraction of the larger, background maternal contribution. Therefore, translating this into the equation shown in FIG. 18, the fetal fraction (k) of the total nucleic acid present in the sample is equal to the equation: k=2×R/(1-2R), where R is the ratio between the Y-chromosome amount and the total amount. Since the Y-chromosome is haploid and Assays for the Total Amount are determined using diploid targets, this calculation is limited to a fetal fraction smaller than 50% of the maternal fraction.

In FIG. 19, a similar calculation for the fetal concentration is shown by using the methylation specific markers (see Assays for Methylation Quantification). In contrast to Y-chromosome specific markers, these markers are from diploid targets, therefore, the limitations stated for the Y-Chromosome Specific Assay can be omitted. Thus, the fetal fraction (k) can be determined using the equation: k=R(1-R), where R is the ratio between the methylation assay and the total assay.

### Simulation

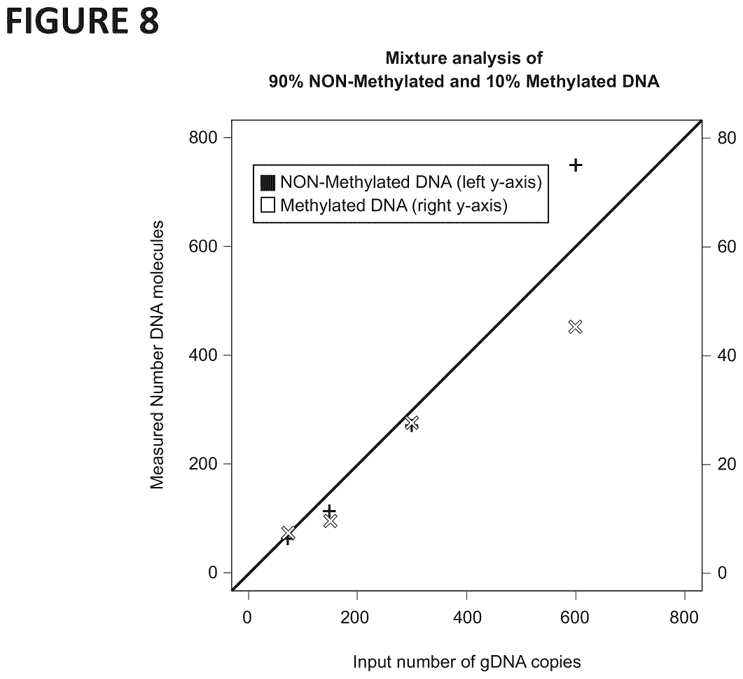

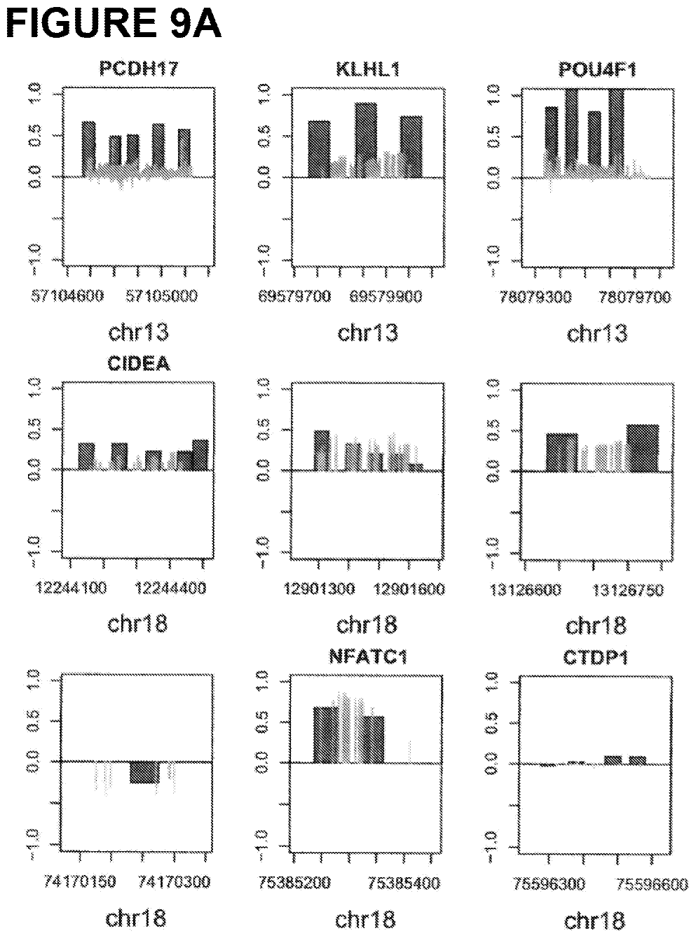

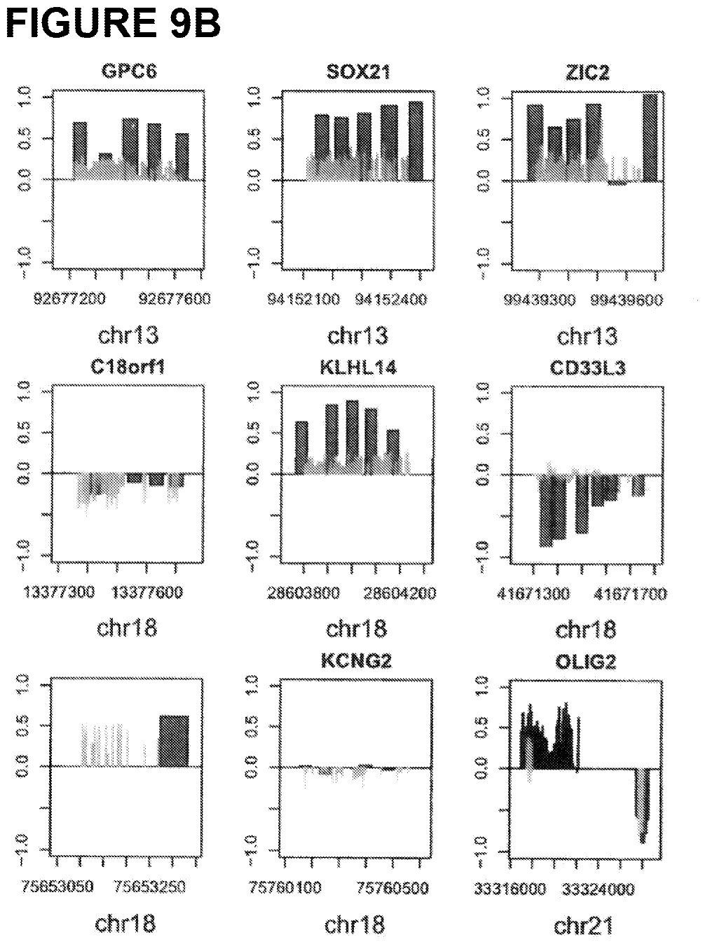

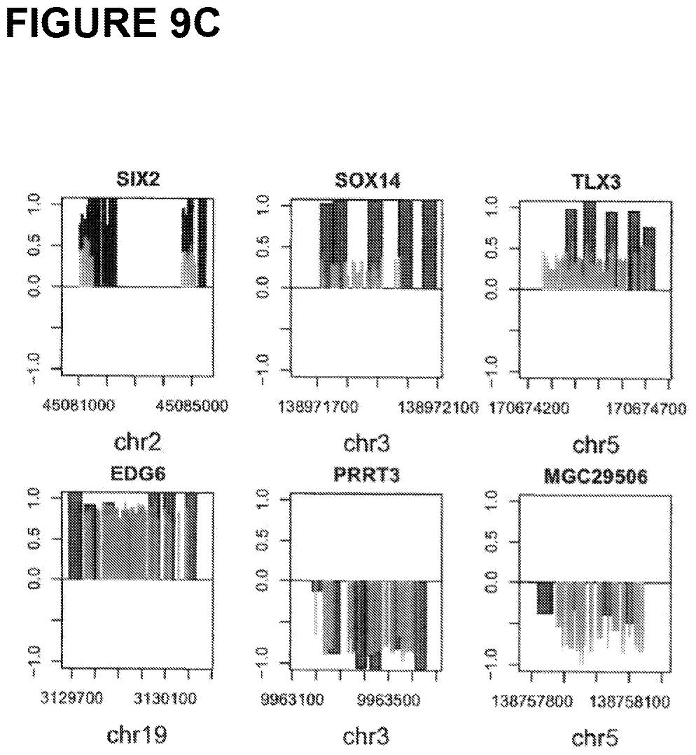

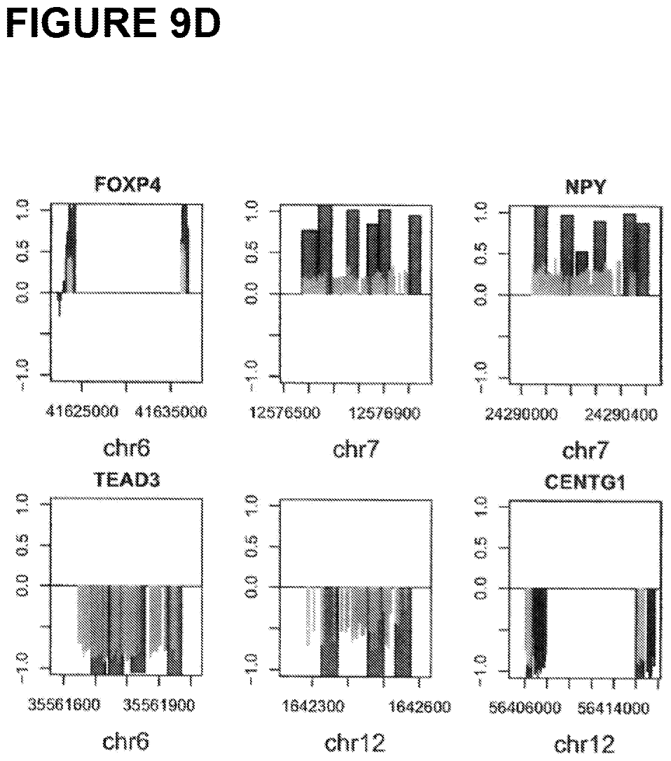

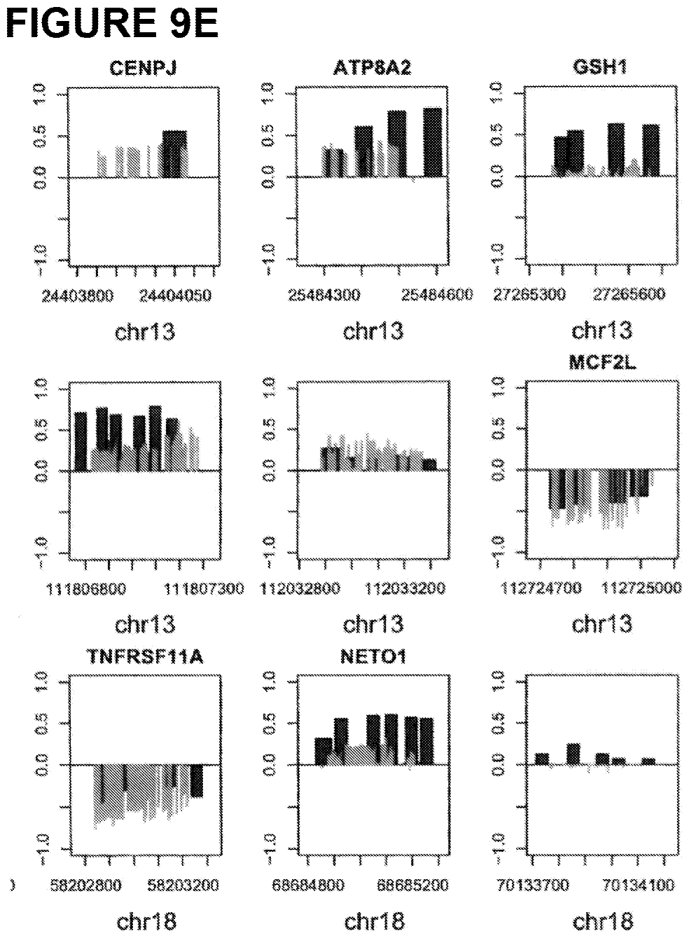

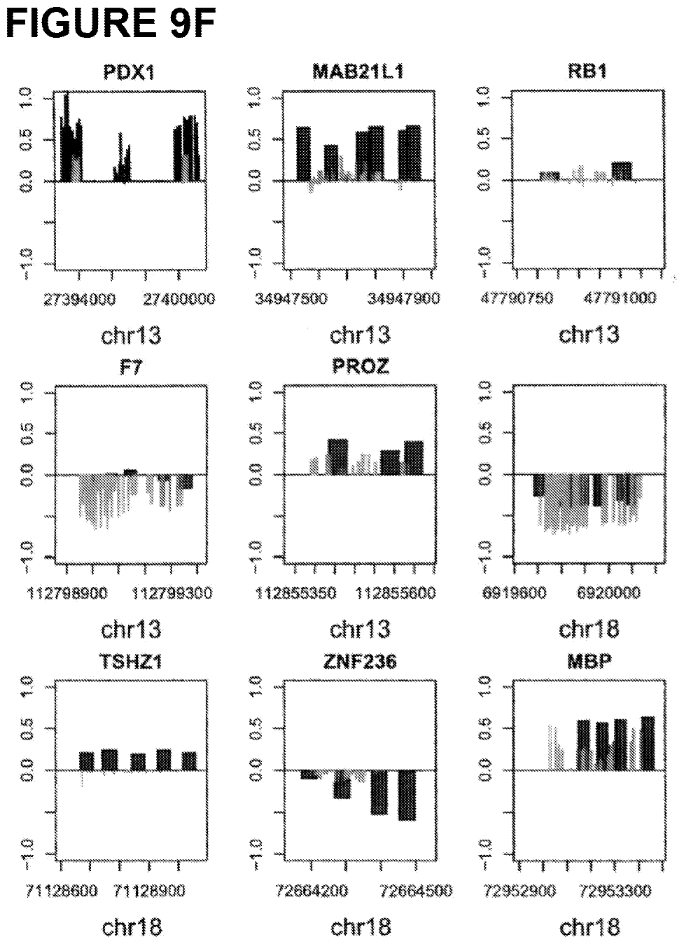

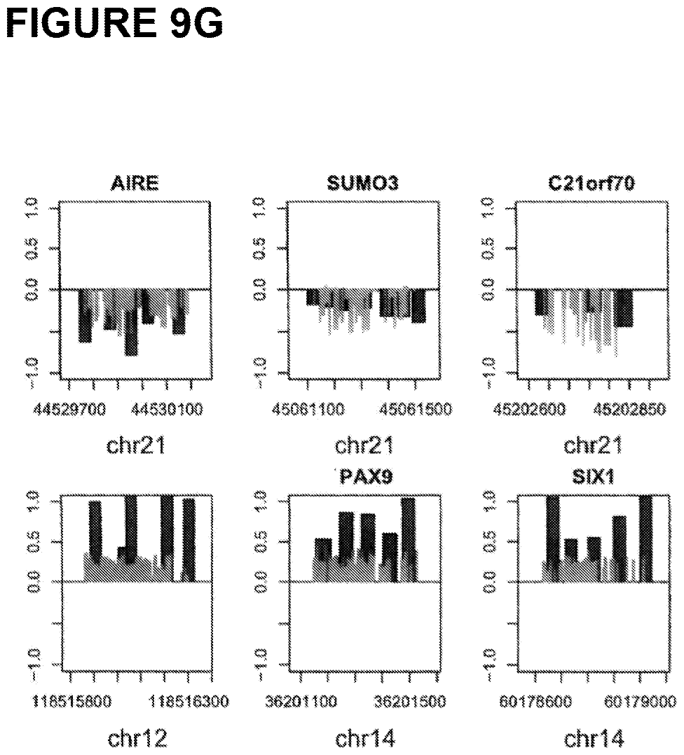

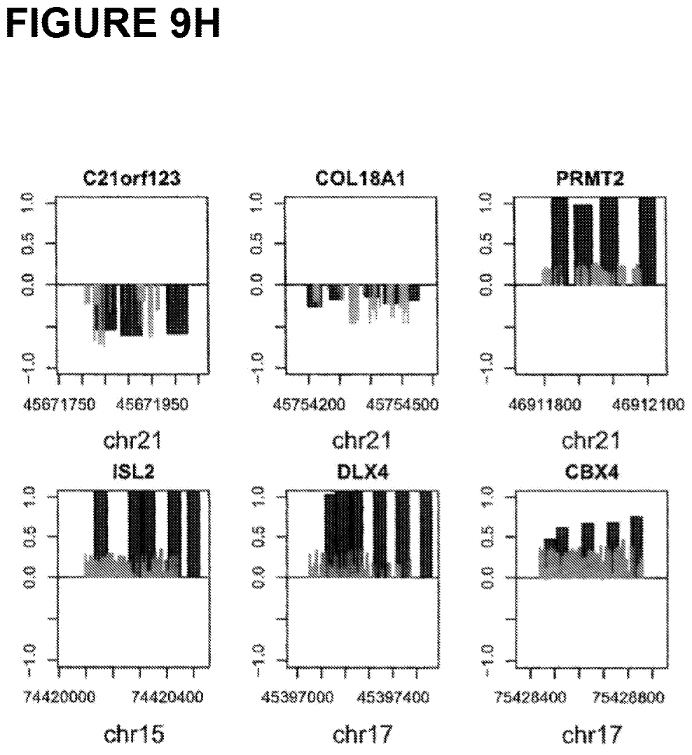

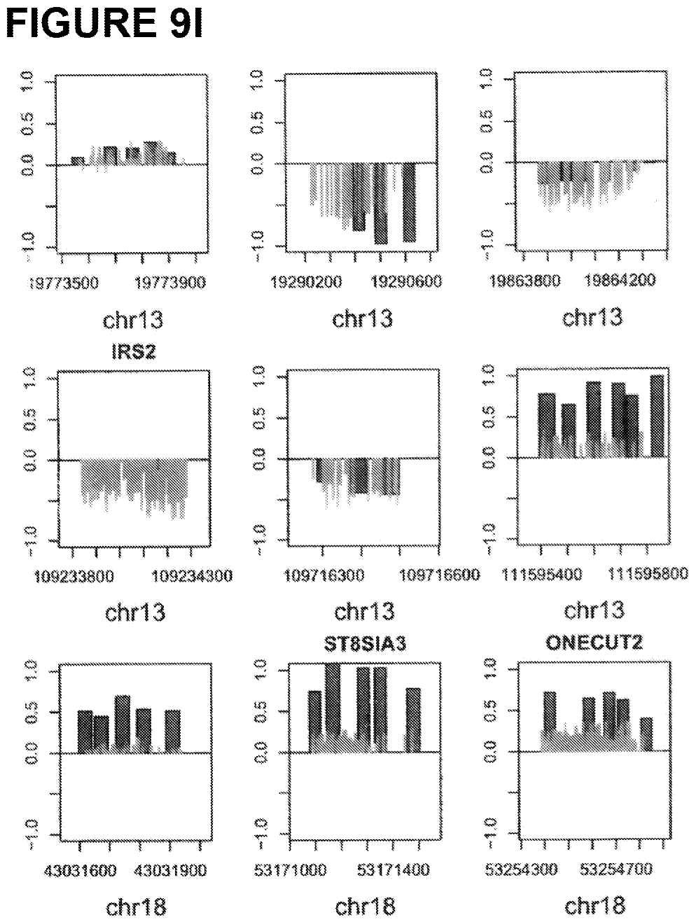

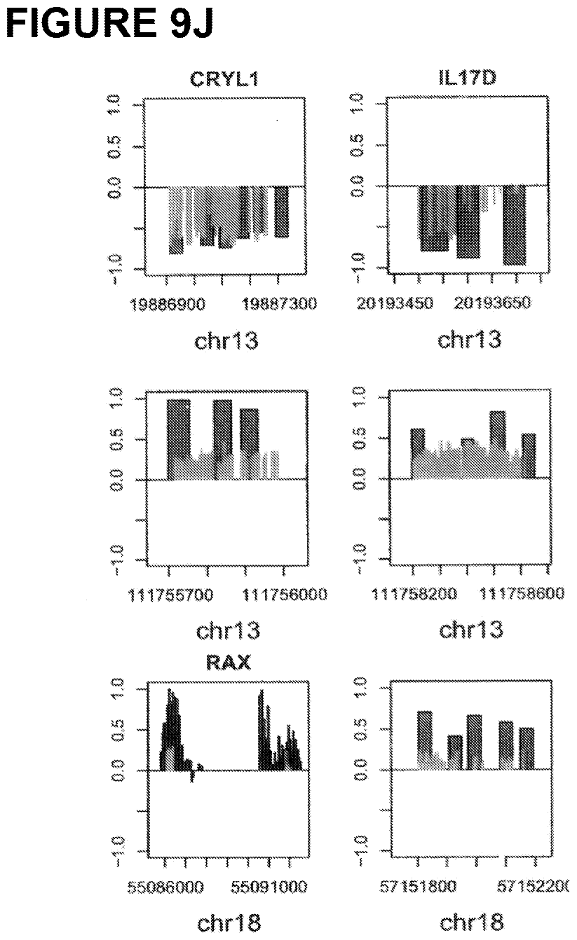

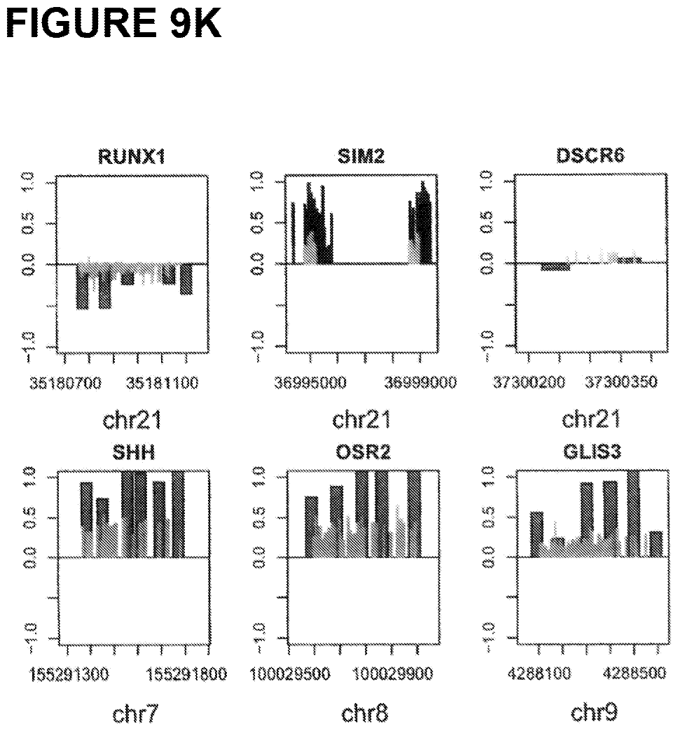

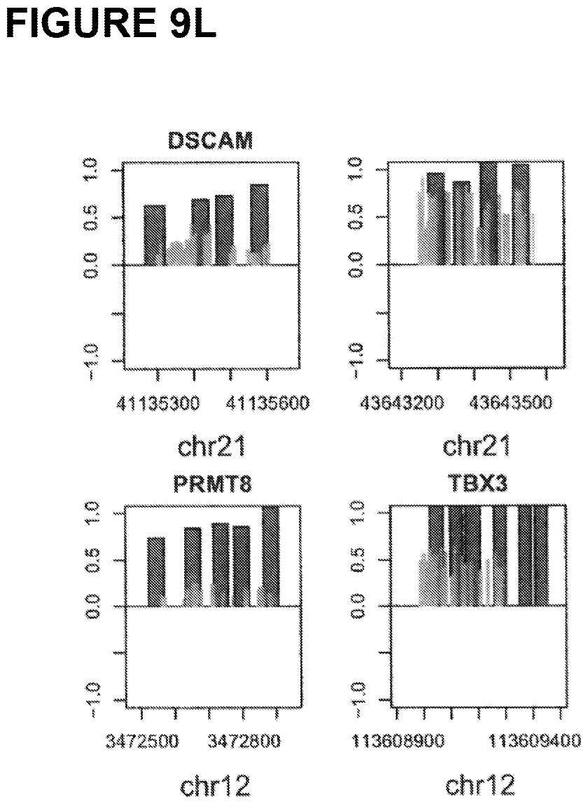

A first simple power calculation was performed that assumes a measurement system that uses 20 markers from chromosome 21, and 20 markers from one or more other autosomes. Starting with 100 copies of fetal DNA, a measurement standard deviation of 25 copies and the probability for a type I error to be lower than 0.001, it was found that the methods of the technology herein will be able to differentiate a diploid from a triploid chromosome set in 99.5% of all cases. The practical implementation of such an approach could for example be achieved using mass spectrometry, a system that uses a competitive PCR approach for absolute copy number measurements. The method can run 20 assays in a single reaction and has been shown to have a standard deviation in repeated measurements of around 3 to 5%. This method was used in combination with known methods for differentiating methylated and non-methylated nucleic acid, for example, using methyl-binding agents to separate nucleic acid or using methylation-sensitive enzymes to digest maternal nucleic acid. FIG. 8 shows the effectiveness of MBD-FC protein (a methyl-binding agent) for capturing and thereby separating methylated DNA in the presence of an excess of unmethylated DNA (see FIG. 8).

A second statistical power analysis was performed to assess the predictive power of an embodiment of the Methylation-Based Fetal Diagnostic Method described herein. The simulation was designed to demonstrate the likelihood of differentiating a group of trisomic chromosome 21 specific markers from a group of reference markers (for example, autosomes excluding chromosome 21). Many parameters influence the ability to discriminate the two populations of markers reliably. For the present simulation, values were chosen for each parameter that have been shown to be the most likely to occur based on experimentation. The following parameters and respective values were used:

### Copy Numbers

Maternal copy numbers=2000
Fetal copy numbers for chromosomes other than 21, X and Y=200
Fetal copy numbers for chromosome 21 in case of euploid fetus=200
Fetal copy numbers for chromosome 21 in case of aneuploid T21 fetus=300
Percent fetal DNA (before methylation-based enrichment)=10% (see above)

### Methylation Frequency

Average methylation percentage in a target region for maternal DNA=10%
Average methylation percentage in a target region for fetal DNA=80%
Average percentage of non-methylated and non-digested maternal DNA (i.e., a function of restriction efficiency (among other things)=5%
Number of assays targeting chromosome 21=10
Number of assays targeting chromosomes other than 21, X and Y=10

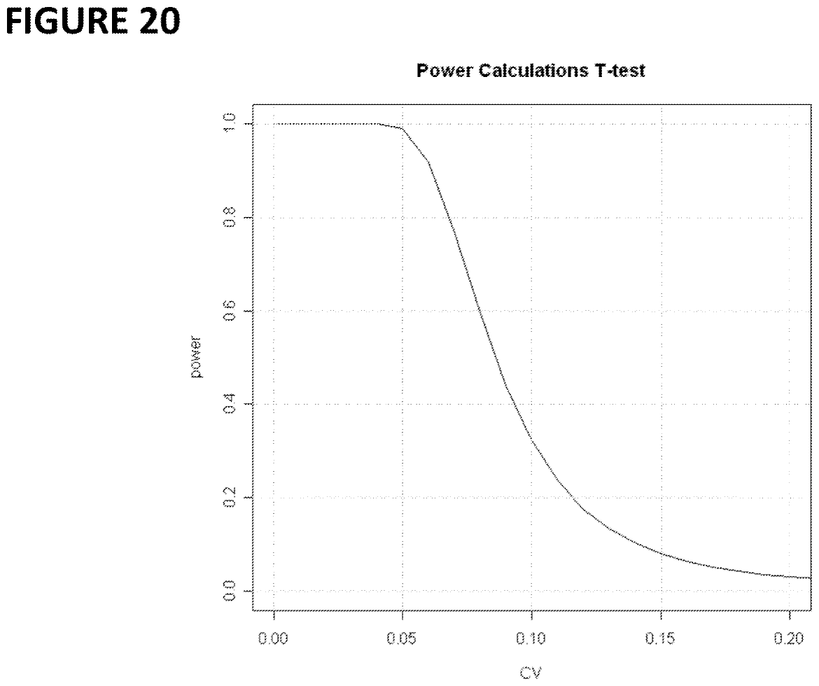

The results are displayed in FIG. 20. Shown is the relationship between the coefficient of variation (CV) on the x-axis and the power to discriminate the assay populations using a simple t-test (y-axis). The data indicates that in 99% of all cases, one can discriminate the two population (euploid vs. aneuploid) on a significance level of 0.001 provided a CV of 5% or less. Based on this simulation, the method represents a powerful noninvasive diagnostic method for the prenatal detection of fetal aneuploidy that is sex-independent and will work in all ethnicities (i.e., no allelic bias).

### Example 3—Additional Differentially-Methylated Targets

### Differentially-Methylated Targets not Located on Chromosome 21

Additional differentially-methylated targets were selected for further analysis based upon previous microarray analysis. See Example 1 for a description of the microarray analysis. During the microarray screen, differentially methylated regions (DMRs) were defined between placenta tissue and PBMC. Regions were selected for EpiTYPER confirmation based upon being hypermethylated in placenta relative to PBMC. After directionality of the change was selected for, regions were chosen based upon statistical significance with regions designed beginning with the most significant and working downward in terms of significance. These studies were performed in eight paired samples of PBMC and placenta. Additional non-chromosome 21 targets are provided in Table 1B, along with a representative genomic sequence from each target in Table 4B.

### Differentially-Methylated Targets Located on Chromosome 21

The microarray screen uncovered only a subset of DMRs located on chromosome 21. The coverage of chromosome 21 by the microarray, however, was insufficient. Therefore a further analysis was completed to examine all 356 CpG islands on chromosome 21 using the standard settings of the UCSC genome browser. As shown in Table 1C below, some of these targets overlapped with those already examined in Table 1A. More specifically, CpG sites located on chromosome 21 including ˜1000 bp upstream and downstream of each CpG was investigated using Sequenom's EpiTYPER® technology. See Example 1, “Validation using Sequenom® EpiTYPER™” for a description of Sequenom's EpiTYPER® technology. These studies were performed in eight paired samples of PBMC and placenta. In addition, since DMRs may also be located outside of defined CpG islands, data mining was performed on publicly available microarray data to identify potential candidate regions with the following characteristics: hypermethylated in placenta relative to maternal blood, not located in a defined CpG island, contained greater than 4 CpG dinucleotides, and contained a recognition sequence for methylation sensitive restriction enzymes. Regions that met these criteria were then examined using Sequenom's EpiTYPER® technology on eight paired PBMC and placenta samples. Additional chromosome 21 targets are provided in Table 1C, along with a representative genomic sequence from each target in Table 4C.

Tables 1B and 1C provide a description of the different targets, including their location and whether they were analyzed during the different phases of analysis, namely microarray analysis, EpiTYPER 8 analysis and EpiTYPER 73 analysis. A “YES” indicates it was analyzed and a “NO” indicates it was not analyzed. The definition of each column in Table 1B and 1C is listed below.
Region Name: Each region is named by the gene(s) residing within the area defined or nearby. Regions where no gene name is listed but rather only contain a locus have no refseq genes in near proximity.
Gene Region: For those regions contained either in close proximity to or within a gene, the gene region further explains the relationship of this region to the nearby gene.
Chrom: The chromosome on which the DMR is located using the hg18 build of the UCSC genome browser.
Start: The starting position of the DMR as designated by the hg18 build of the UCSC genome browser.
End: The ending position of the DMR as designated by the hg18 build of the UCSC genome browser.
Microarray Analysis: Describes whether this region was also/initially determined to be differentially methylated by microarray analysis. The methylated fraction of ten paired placenta and PBMC samples was isolated using the MBD-Fc protein. The two tissue fractions were then labeled with either Alexa Fluor 555-aha-dCTP (PBMC) or Alexa Fluor 647-aha-dCTP (placental) using the BioPrime Total Genomic Labeling System™ and hybridized to Agilent® CpG Island microarrays. Many regions examined in these studies were not contained on the initial microarray.
EpiTYPER 8 Samples: Describes whether this region was analyzed and determined to be differentially methylated in eight paired samples of placenta and peripheral blood mononuclear cells (PBMC) using EpiTYPER technology. Regions that were chosen for examination were based on multiple criteria. First, regions were selected based on data from the Microarray Analysis. Secondly, a comprehensive examination of all CpG islands located on chromosome 21 was undertaken. Finally, selected regions on chromosome 21 which had lower CpG frequency than those located in CpG islands were examined.
EpiTYPER 73 Samples: Describes whether this region was subsequently analyzed using EpiTYPER technology in a sample cohort consisting of 73 paired samples of placenta and PBMC. All regions selected for analysis in this second sample cohort were selected based on the results from the experimentation described in the EpiTYPER 8 column. More specifically, the regions in this additional cohort exhibited a methylation profile similar to that determined in the EpiTYPER 8 Samples analysis. For example, all of the regions listed in Tables 1B-1C exhibit different levels of DNA methylation in a significant portion of the examined CpG dinucleotides within the defined region. Differential DNA methylation of CpG sites was determined using a paired T Test with those sites considered differentially methylated if the p-value (when comparing placental tissue to PBMC) is p<0.05.
Previously Validated EpiTYPER: Describes whether this region or a portion of this region was validated using EpiTYPER during previous experimentation. (See Examples 1 and 2).
Relative Methylation Placenta to Maternal: Describes the direction of differential methylation. Regions labeled as “hypermethylation” are more methylated within the designated region in placenta samples relative to PBMC and “hypomethylation” are more methylated within the designated region in PBMC samples.

TABLE 1A

| | | | | | MEAN LOG |
|---|---|---|---|---|---|
| | | | | | RATIO MICRO- |
| GENE NAME | CHROM | START | END | CpG ISLAND | ARRAY |
| chr13 group00016 | chr13 | 19773745 | 19774050 | chr13: 19773518-19774214 | 0.19 |
| chr13 group00005 | chr13 | 19290394 | 19290768 | :— | −0.89 |
| CRYL1 | chr13 | 19887090 | 19887336 | chr13: 19887007-19887836 | −0.63 |
| IL17D | chr13 | 20193675 | 20193897 | chr13: 20193611-20194438 | −1.01 |
| CENPJ | chr13 | 24404023 | 24404359 | :— | 0.57 |
| ATP8A2 | chr13 | 25484475 | 25484614 | chr13: 25484287-25484761 | 0.81 |
| GSH1 | chr13 | 27265542 | 27265834 | chr13: 27264549-27266505 | 0.57 |
| PDX1 | chr13 | 27393789 | 27393979 | chr13: 27392001-27394099 | 0.55 |
| PDX1 | chr13 | 27400459 | 27401165 | chr13: 27400362-27400744; | 0.73 |
| | | | | chr13: 27401057-27401374 | |
| MAB21L1 | chr13 | 34947737 | 34948062 | chr13: 34947570-34948159 | 0.66 |
| RB1 | chr13 | 47790983 | 47791646 | chr13: 47790636-47791858 | 0.18 |
| PCDH17 | chr13 | 57104856 | 57106841 | chr13: 57104527-57106931 | 0.46 |
| KLHL1 | chr13 | 69579933 | 69580146 | chr13: 69579733-69580220 | 0.79 |
| POU4F1 | chr13 | 78079515 | 78081073 | chr13: 78079328-78079615; | 0.66 |
| | | | | chr13: 78080860-78081881 | |
| GPC6 | chr13 | 92677402 | 92678666 | chr13: 92677246-92678878 | 0.66 |
| SOX21 | chr13 | 94152286 | 94153047 | chr13: 94152190-94153185 | 0.94 |
| ZIC2 | chr13 | 99439660 | 99440858 | chr13: 99439335-99440189; | 0.89 |
| | | | | chr13: 99440775-99441095 | |
| IRS2 | chr13 | 109232856 | 109235065 | chr13: 109232467-109238181 | −0.17 |
| chr13 group00350 | chr13 | 109716455 | 109716604 | chr13: 109716325-109716726 | −0.37 |
| chr13 group00385 | chr13 | 111595578 | 111595955 | chr13: 111595459-111596131 | 0.87 |
| chr13 group00390 | chr13 | 111756337 | 111756593 | chr13: 111755805-111756697 | 0.71 |
| chr13 group00391 | chr13 | 111759856 | 111760045 | chr13: 111757885-111760666 | 0.86 |
| chr13 group00395 | chr13 | 111808255 | 111808962 | chr13: 111806599-111808492; | 0.96 |
| | | | | chr13: 111808866-111809114 | |
| chr13 group00399 | chr13 | 112033503 | 112033685 | chr13: 112032967-112033734 | 0.38 |
| MCF2L | chr13 | 112724910 | 112725742 | chr13: 112724782-112725121; | −0.47 |
| | | | | chr13: 112725628-112725837 | |
| F7 | chr13 | 112799123 | 112799379 | chr13: 112798487-112799566 | −0.05 |
| PROZ | chr13 | 112855566 | 112855745 | chr13: 112855289-112855866 | 0.29 |
| chr18 group00039 | chr18 | 6919797 | 6919981 | chr18: 6919450-6920088 | −0.38 |
| CIDEA | chr18 | 12244327 | 12244696 | chr18: 12244147-12245089 | 0.23 |
| chr18 group00091 | chr18 | 12901467 | 12901643 | chr18: 12901024-12902704 | 0.16 |
| chr18 group00094 | chr18 | 13126819 | 13126986 | chr18: 13126596-13127564 | 0.41 |
| C18orf1 | chr18 | 13377536 | 13377654 | chr18: 13377385-13377686 | −0.12 |
| KLHL14 | chr18 | 28603978 | 28605183 | chr18: 28603688-28606300 | 0.83 |
| CD33L3 | chr18 | 41671477 | 41673011 | chr18: 41671386-41673101 | −0.34 |
| ST8SIA3 | chr18 | 53171265 | 53171309 | chr18: 53170705-53172603 | 1.02 |
| ONECUT2 | chr18 | 53254808 | 53259810 | chr18: 53254152-53259851 | 0.74 |
| RAX | chr18 | 55086286 | 55086436 | chr18: 55085813-55087807 | 0.88 |
| chr18 group00277 | chr18 | 57151972 | 57152311 | chr18: 57151663-57152672 | 0.58 |
| TNFRSF11A | chr18 | 58203013 | 58203282 | chr18: 58202849-58203367 | −0.33 |
| NETO1 | chr18 | 68685099 | 68687060 | chr18: 68684945-68687851 | 0.65 |
| chr18 group00304 | chr18 | 70133945 | 70134397 | chr18: 70133732-70134724 | 0.12 |
| TSHZ1 | chr18 | 71128742 | 71128974 | chr18: 71128638-71129076 | 0.23 |
| ZNF236 | chr18 | 72664454 | 72664736 | chr18: 72662797-72664893 | −0.62 |
| MBP | chr18 | 72953150 | 72953464 | chr18: 72953137-72953402 | 0.6 |
| chr18 group00342 | chr18 | 74170347 | 74170489 | chr18: 74170210-74170687 | −0.2 |
| NFATC1 | chr18 | 75385424 | 75386008 | chr18: 75385279-75386532 | 0.23 |
| CTDP1 | chr18 | 75596358 | 75596579 | chr18: 75596009-75596899 | 0.07 |
| chr18 group00430 | chr18 | 75653272 | 75653621 | :— | 0.52 |
| KCNG2 | chr18 | 75760343 | 75760820 | chr18: 75759900-75760988 | 0.01 |
| OLIG2 | chr21 | 33317673 | 33321183 | chr21: 33316998-33322115 | 0.66 |
| OLIG2 | chr21 | 33327593 | 33328334 | chr21: 33327447-33328408 | −0.75 |
| RUNX1 | chr21 | 35180938 | 35185436 | chr21: 35180822-35181342; | −0.68 |
| | | | | chr21: 35182320-35185557 | |
| SIM2 | chr21 | 36994965 | 36995298 | chr21: 36990063-36995761 | 0.83 |
| SIM2 | chr21 | 36999025 | 36999410 | chr21: 36998632-36999555 | 0.87 |
| DSCR6 | chr21 | 37300407 | 37300512 | chr21: 37299807-37301307 | 0.22 |
| DSCAM | chr21 | 41135559 | 41135706 | chr21: 41135380-41135816 | 1.03 |
| chr21 group00165 | chr21 | 43643421 | 43643786 | chr21: 43643322-43643874 | 1.14 |
| AIRE | chr21 | 44529935 | 44530388 | chr21: 44529856-44530472 | −0.55 |
| SUMO3 | chr21 | 45061293 | 45061853 | chr21: 45061154-45063386 | −0.41 |
| C21orf70 | chr21 | 45202815 | 45202972 | chr21: 45202706-45203073 | −0.46 |
| C21orf123 | chr21 | 45671984 | 45672098 | chr21: 45671933-45672201 | −0.63 |
| COL18A1 | chr21 | 45754383 | 45754487 | chr21: 45753653-45754639 | −0.18 |
| PRMT2 | chr21 | 46911967 | 46912385 | chr21: 46911628-46912534 | 1.08 |
| SIX2 | chr2 | 45081223 | 45082129 | chr2: 45081148-45082287 | 1.15 |
| SIX2 | chr2 | 45084851 | 45085711 | chr2: 45084715-45084986; | 1.21 |
| | | | | chr2: 45085285-45086054 | |
| SOX14 | chr3 | 138971870 | 138972322 | chr3: 138971738-138972096; | 1.35 |
| | | | | chr3: 138972281-138973691 | |
| TLX3 | chr5 | 170674439 | 170676431 | chr5: 170674208-170675356; | 0.91 |
| | | | | chr5: 170675783-170676712 | |
| FOXP4 | chr6 | 41623666 | 41624114 | chr6: 41621630-41624167 | 1.1 |
| FOXP4 | chr6 | 41636384 | 41636779 | chr6: 41636244-41636878 | 1.32 |
| chr7 group00267 | chr7 | 12576755 | 12577246 | chr7: 12576690-12577359 | 0.94 |
| NPY | chr7 | 24290224 | 24291508 | chr7: 24290083-24291605 | 0.93 |
| SHH | chr7 | 155291537 | 155292091 | chr7: 155288453-155292175 | 0.98 |
| OSR2 | chr8 | 100029764 | 100030536 | chr8: 100029673-100030614 | 1.21 |
| GLIS3 | chr9 | 4288283 | 4289645 | chr9: 4287817-4290182 | 1.24 |
| PRMT8 | chr12 | 3472714 | 3473190 | chr12: 3470227-3473269 | 0.86 |
| TBX3 | chr12 | 113609153 | 113609453 | chr12: 113609112-113609535 | 1.45 |
| chr12 group00801 | chr12 | 118516189 | 118517435 | chr12: 118515877-118517595 | 1.1 |
| PAX9 | chr14 | 36201402 | 36202386 | chr14: 36200932-36202536 | 0.89 |
| SIX1 | chr14 | 60178801 | 60179346 | chr14: 60178707-60179539 | 0.95 |
| ISL2 | chr15 | 74420013 | 74421546 | chr15: 74419317-74422570 | 1.08 |
| DLX4 | chr17 | 45397228 | 45397930 | chr17: 45396281-45398063 | 1.25 |
| CBX4 | chr17 | 75428613 | 75431793 | chr17: 75427586-75433676 | 1 |
| EDG6 | chr19 | 3129836 | 3130874 | chr19: 3129741-3130986 | 1.35 |
| PRRT3 | chr3 | 9963364 | 9964023 | chr3: 9962895-9964619 | −0.85 |
| MGC29506 | chr5 | 138757911 | 138758724 | chr5: 138755609-138758810 | −0.63 |
| TEAD3 | chr6 | 35561812 | 35562252 | chr6: 35561754-35562413 | −1.17 |
| chr12 group00022 | chr12 | 1642456 | 1642708 | chr12: 1642195-1642774 | −1.33 |
| CENTG1 | chr12 | 56406249 | 56407788 | chr12: 56406176-56407818 | −1.07 |
| CENTG1 | chr12 | 56416146 | 56418794 | chr12: 56416095-56416628; | −0.94 |
| | | | | chr12: 56418745-56419001 | |

| | MEAN | MEAN | METHYLATION | RELATIVE |
|---|---|---|---|---|
| | MATERNAL | PLACENTA | DIFFERENCE | METHYLATION |
| | METHYLATION | METHYLATION | PLACENTA − | PLACENTA TO |
| GENE NAME | EPITYPER | EPITYPER | MATERNAL | MATERNAL |
| chr13 group00016 | 0.22 | 0.32 | 0.1 | HYPERMETHYLATION |
| chr13 group00005 | 0.94 | 0.35 | −0.59 | HYPOMETHYLATION |
| CRYL1 | 0.74 | 0.21 | −0.53 | HYPOMETHYLATION |
| IL17D | 0.53 | 0.13 | −0.39 | HYPOMETHYLATION |
| CENPJ | 0.17 | 0.49 | 0.32 | HYPERMETHYLATION |
| ATP8A2 | 0.16 | 0.43 | 0.27 | HYPERMETHYLATION |
| GSH1 | 0.13 | 0.19 | 0.05 | HYPERMETHYLATION |
| PDX1 | 0.06 | 0.2 | 0.14 | HYPERMETHYLATION |
| PDX1 | 0.12 | 0.26 | 0.14 | HYPERMETHYLATION |
| MAB21L1 | 0.11 | 0.17 | 0.06 | HYPERMETHYLATION |
| RB1 | 0.45 | 0.48 | 0.03 | HYPERMETHYLATION |
| PCDH17 | 0.15 | 0.21 | 0.06 | HYPERMETHYLATION |
| KLHL1 | 0.09 | 0.28 | 0.2 | HYPERMETHYLATION |
| POU4F1 | 0.12 | 0.23 | 0.11 | HYPERMETHYLATION |
| GPC6 | 0.06 | 0.19 | 0.13 | HYPERMETHYLATION |
| SOX21 | 0.16 | 0.4 | 0.25 | HYPERMETHYLATION |
| ZIC2 | 0.13 | 0.35 | 0.22 | HYPERMETHYLATION |
| IRS2 | 0.73 | 0.38 | −0.35 | HYPOMETHYLATION |
| chr13 group00350 | 0.77 | 0.41 | −0.36 | HYPOMETHYLATION |
| chr13 group00385 | 0.06 | 0.2 | 0.14 | HYPERMETHYLATION |
| chr13 group00390 | 0.12 | 0.34 | 0.22 | HYPERMETHYLATION |
| chr13 group00391 | 0.11 | 0.36 | 0.25 | HYPERMETHYLATION |
| chr13 group00395 | 0.13 | 0.35 | 0.22 | HYPERMETHYLATION |
| chr13 group00399 | 0.26 | 0.43 | 0.18 | HYPERMETHYLATION |
| MCF2L | 0.91 | 0.33 | −0.58 | HYPOMETHYLATION |
| F7 | 0.97 | 0.55 | −0.41 | HYPOMETHYLATION |
| PROZ | 0.15 | 0.3 | 0.16 | HYPERMETHYLATION |
| chr18 group00039 | 0.88 | 0.39 | −0.49 | HYPOMETHYLATION |
| CIDEA | 0.14 | 0.23 | 0.1 | HYPERMETHYLATION |
| chr18 group00091 | 0.15 | 0.43 | 0.29 | HYPERMETHYLATION |
| chr18 group00094 | 0.07 | 0.34 | 0.27 | HYPERMETHYLATION |
| C18orf1 | 0.95 | 0.69 | −0.26 | HYPOMETHYLATION |
| KLHL14 | 0.07 | 0.19 | 0.12 | HYPERMETHYLATION |
| CD33L3 | 0.49 | 0.44 | −0.05 | HYPOMETHYLATION |
| ST8SIA3 | 0.09 | 0.25 | 0.16 | HYPERMETHYLATION |
| ONECUT2 | 0.09 | 0.23 | 0.14 | HYPERMETHYLATION |
| RAX | 0.11 | 0.26 | 0.16 | HYPERMETHYLATION |
| chr18 group00277 | 0.08 | 0.21 | 0.13 | HYPERMETHYLATION |
| TNFRSF11A | 0.88 | 0.28 | −0.6 | HYPOMETHYLATION |
| NETO1 | 0.09 | 0.22 | 0.13 | HYPERMETHYLATION |
| chr18 group00304 | 0.93 | 0.92 | −0.01 | NOT CONFIRMED |
| TSHZ1 | 0.95 | 0.92 | −0.03 | NOT CONFIRMED |
| ZNF236 | 0.17 | 0.1 | −0.07 | HYPOMETHYLATION |
| MBP | 0.44 | 0.72 | 0.28 | HYPERMETHYLATION |
| chr18 group00342 | 0.78 | 0.48 | −0.3 | HYPOMETHYLATION |
| NFATC1 | 0.14 | 0.84 | 0.7 | HYPERMETHYLATION |
| CTDP1 | 0.97 | 0.96 | −0.01 | NOT CONFIRMED |
| chr18 group00430 | 0.24 | 0.62 | 0.39 | HYPERMETHYLATION |
| KCNG2 | 0.84 | 0.75 | −0.09 | NOT CONFIRMED |
| OLIG2 | 0.11 | 0.2 | 0.09 | HYPERMETHYLATION |
| OLIG2 | 0.77 | 0.28 | −0.49 | HYPOMETHYLATION |
| RUNX1 | 0.14 | 0.07 | −0.07 | HYPOMETHYLATION |
| SIM2 | 0.08 | 0.26 | 0.18 | HYPERMETHYLATION |
| SIM2 | 0.06 | 0.24 | 0.18 | HYPERMETHYLATION |
| DSCR6 | 0.04 | 0.14 | 0.11 | HYPERMETHYLATION |
| DSCAM | 0.06 | 0.29 | 0.23 | HYPERMETHYLATION |
| chr21 group00165 | 0.16 | 0.81 | 0.65 | HYPERMETHYLATION |
| AIRE | 0.62 | 0.27 | −0.35 | HYPOMETHYLATION |
| SUMO3 | 0.55 | 0.46 | −0.09 | HYPOMETHYLATION |
| C21orf70 | 0.96 | 0.51 | −0.46 | HYPOMETHYLATION |
| C21orf123 | 0.92 | 0.43 | −0.49 | HYPOMETHYLATION |
| COL18A1 | 0.97 | 0.72 | −0.25 | HYPOMETHYLATION |
| PRMT2 | 0.04 | 0.25 | 0.21 | HYPERMETHYLATION |
| SIX2 | 0.08 | 0.36 | 0.28 | HYPERMETHYLATION |
| SIX2 | 0.07 | 0.35 | 0.28 | HYPERMETHYLATION |
| SOX14 | 0.08 | 0.33 | 0.25 | HYPERMETHYLATION |
| TLX3 | 0.11 | 0.35 | 0.24 | HYPERMETHYLATION |
| FOXP4 | 0.07 | 0.27 | 0.2 | HYPERMETHYLATION |
| FOXP4 | 0.04 | 0.33 | 0.29 | HYPERMETHYLATION |
| chr7 group00267 | 0.08 | 0.26 | 0.17 | HYPERMETHYLATION |
| NPY | 0.09 | 0.3 | 0.21 | HYPERMETHYLATION |
| SHH | 0.19 | 0.52 | 0.33 | HYPERMETHYLATION |
| OSR2 | 0.08 | 0.43 | 0.35 | HYPERMETHYLATION |
| GLIS3 | 0.06 | 0.24 | 0.18 | HYPERMETHYLATION |
| PRMT8 | 0.07 | 0.23 | 0.16 | HYPERMETHYLATION |
| TBX3 | 0.09 | 0.56 | 0.48 | HYPERMETHYLATION |
| chr12 group00801 | 0.06 | 0.25 | 0.19 | HYPERMETHYLATION |
| PAX9 | 0.11 | 0.32 | 0.21 | HYPERMETHYLATION |
| SIX1 | 0.1 | 0.33 | 0.22 | HYPERMETHYLATION |
| ISL2 | 0.08 | 0.27 | 0.19 | HYPERMETHYLATION |
| DLX4 | 0.1 | 0.32 | 0.22 | HYPERMETHYLATION |
| CBX4 | 0.07 | 0.27 | 0.21 | HYPERMETHYLATION |
| EDG6 | 0.04 | 0.87 | 0.83 | HYPERMETHYLATION |
| PRRT3 | 0.9 | 0.09 | −0.81 | HYPOMETHYLATION |
| MGC29506 | 0.93 | 0.17 | −0.76 | HYPOMETHYLATION |
| TEAD3 | 0.92 | 0.13 | −0.8 | HYPOMETHYLATION |
| chr12 group00022 | 0.66 | 0.09 | −0.57 | HYPOMETHYLATION |
| CENTG1 | 0.95 | 0.19 | −0.77 | HYPOMETHYLATION |
| CENTG1 | 0.85 | 0.16 | −0.69 | HYPOMETHYLATION |

Information in Table 1A based on the March 2006 human reference sequence (NCBI Build 36.1), which was produced by the International Human Genome Sequencing Consortium.

TABLE 1B
Non-Chromosome 21 differentially methylated regions

| | | | | | Micro- | EpiTYPER | EpiTYPER | Previously | Relative Methyl- |
|---|---|---|---|---|---|---|---|---|---|
| | Gene | | | | array | 8 | 73 | Validated | ation Placenta |
| Region Name | Region | Chrom | Start | End | Analysis | Samples | Samples | EpiTYPER | to Maternal |
| TFAP2E | Intron | chr1 | 35815000 | 35816200 | YES | YES | NO | NO | Hypermethylation |
| LRRC8D | Intron/Exon | chr1 | 90081350 | 90082250 | YES | YES | NO | NO | Hypermethylation |
| TBX15 | Promoter | chr1 | 119333500 | 119333700 | YES | YES | NO | NO | Hypermethylation |
| C1orf51 | Upstream | chr1 | 148520900 | 148521300 | YES | YES | NO | NO | Hypermethylation |
| chr1: 179553900- | Intergenic | chr1 | 179553900 | 179554600 | YES | YES | NO | NO | Hypermethylation |
| 179554600 | | | | | | | | | |
| ZFP36L2 | Exon | chr2 | 43304900 | 43305100 | YES | YES | NO | NO | Hypermethylation |
| SIX2 | Downstream | chr2 | 45081000 | 45086000 | YES | YES | NO | YES | Hypermethylation |
| chr2: 137238500- | Intergenic | chr2 | 137238500 | 137240000 | YES | YES | NO | NO | Hypermethylation |
| 137240000 | | | | | | | | | |
| MAP1D | Intron/Exon | chr2 | 172652800 | 172653600 | YES | YES | NO | NO | Hypermethylation |
| WNT6 | Intron | chr2 | 219444250 | 219444290 | YES | YES | NO | NO | Hypermethylation |
| INPP5D | Promoter | chr2 | 233633200 | 233633700 | YES | YES | YES | NO | Hypermethylation |
| chr2: 241211100- | Intergenic | chr2 | 241211100 | 241211600 | YES | YES | YES | NO | Hypermethylation |
| 241211600 | | | | | | | | | |
| WNT5A | Intron | chr3 | 55492550 | 55492850 | YES | YES | NO | NO | Hypermethylation |
| chr3: 138971600- | Intergenic | chr3 | 138971600 | 138972200 | YES | YES | YES | YES | Hypermethylation |
| 138972200 | | | | | | | | | |
| ZIC4 | Intron | chr3 | 148598200 | 148599000 | YES | YES | NO | NO | Hypermethylation |
| FGF12 | Intron/Exon | chr3 | 193608500 | 193610500 | YES | YES | NO | NO | Hypermethylation |
| GP5 | Exon | chr3 | 195598400 | 195599200 | YES | YES | NO | NO | Hypermethylation |
| MSX1 | Upstream | chr4 | 4910550 | 4911100 | YES | YES | NO | NO | Hypermethylation |
| NKX3-2 | Intron/Exon | chr4 | 13152500 | 13154500 | YES | YES | NO | NO | Hypermethylation |
| chr4: 111752000- | Intergenic | chr4 | 111752000 | 111753000 | YES | YES | YES | NO | Hypermethylation |
| 111753000 | | | | | | | | | |
| SFRP2 | Promoter | chr4 | 154928800 | 154930100 | YES | YES | NO | NO | Hypermethylation |
| chr4: 174664300- | Intergenic | chr4 | 174664300 | 174664800 | YES | YES | NO | NO | Hypermethylation |
| 174664800 | | | | | | | | | |
| chr4: 174676300- | Intergenic | chr4 | 174676300 | 174676800 | YES | YES | NO | NO | Hypermethylation |
| 174676800 | | | | | | | | | |
| SORBS2 | Intron | chr4 | 186796900 | 186797500 | YES | YES | NO | NO | Hypermethylation |
| chr5: 42986900- | Intergenic | chr5 | 42986900 | 42988200 | YES | YES | NO | NO | Hypermethylation |
| 42988200 | | | | | | | | | |
| chr5: 72712000- | Intergenic | chr5 | 72712000 | 72714100 | YES | YES | NO | NO | Hypermethylation |
| 72714100 | | | | | | | | | |
| chr5: 72767550- | Intergenic | chr5 | 72767550 | 72767800 | YES | YES | NO | NO | Hypermethylation |
| 72767800 | | | | | | | | | |
| NR2F1 | Intron/Exon | chr5 | 92955000 | 92955250 | YES | YES | NO | NO | Hypermethylation |
| PCDHGA1 | Intron | chr5 | 140850500 | 140852500 | YES | YES | YES | NO | Hypermethylation |
| chr6: 10489100- | Intergenic | chr6 | 10489100 | 10490200 | YES | YES | YES | NO | Hypermethylation |
| 10490200 | | | | | | | | | |
| FOXP4 | Intron | chr6 | 41636200 | 41637000 | YES | YES | NO | YES | Hypermethylation |
| chr7: 19118400- | Intergenic | chr7 | 19118400 | 19118700 | YES | YES | NO | NO | Hypermethylation |
| 19118700 | | | | | | | | | |
| chr7: 27258000- | Intergenic | chr7 | 27258000 | 27258400 | YES | YES | NO | NO | Hypermethylation |
| 27258400 | | | | | | | | | |
| TBX20 | Upstream | chr7 | 35267500 | 35268300 | YES | YES | NO | NO | Hypermethylation |
| AGBL3 | Promoter | chr7 | 134321300 | 134322300 | YES | YES | NO | NO | Hypermethylation |
| XPO7 | Downstream | chr8 | 21924000 | 21924300 | YES | YES | NO | NO | Hypermethylation |
| chr8: 41543400- | Intergenic | chr8 | 41543400 | 41544000 | YES | YES | NO | NO | Hypermethylation |
| 41544000 | | | | | | | | | |
| GDF6 | Exon | chr8 | 97225400 | 97227100 | YES | YES | NO | NO | Hypermethylation |
| OSR2 | Intron/Exon | chr8 | 100029000 | 100031000 | YES | YES | YES | YES | Hypermethylation |
| GLIS3 | Intron/Exon | chr9 | 4288000 | 4290000 | YES | YES | NO | YES | Hypermethylation |
| NOTCH1 | Intron | chr9 | 138547600 | 138548400 | YES | YES | YES | NO | Hypermethylation |
| EGFL7 | Upstream | chr9 | 138672350 | 138672850 | YES | YES | NO | NO | Hypermethylation |
| CELF2 | Intron/Exon | chr10 | 11246700 | 11247900 | YES | YES | NO | NO | Hypermethylation |
| HHEX | Intron | chr10 | 94441000 | 94441800 | YES | YES | NO | NO | Hypermethylation |
| DOCK1/FAM196A | Intron/Exon | chr10 | 128883000 | 128883500 | YES | YES | NO | NO | Hypermethylation |
| PAX6 | Intron | chr11 | 31782400 | 31783500 | YES | YES | NO | NO | Hypermethylation |
| FERMT3 | Intron/Exon | chr11 | 63731200 | 63731700 | YES | YES | YES | NO | Hypermethylation |
| PKNOX2 | Intron | chr11 | 124541200 | 124541800 | YES | YES | NO | NO | Hypermethylation |
| KIRREL3 | Intron | chr11 | 126375150 | 126375300 | YES | YES | NO | NO | Hypermethylation |
| BCAT1 | Intron | chr12 | 24946700 | 24947600 | YES | YES | NO | NO | Hypermethylation |
| HOXC13 | Intron/Exon | chr12 | 52625000 | 52625600 | YES | YES | NO | NO | Hypermethylation |
| TBX5 | Promoter | chr12 | 113330500 | 113332000 | YES | YES | NO | NO | Hypermethylation |
| TBX3 | Upstream | chr12 | 113609000 | 113609500 | YES | YES | NO | YES | Hypermethylation |
| chr12: 113622100- | Intergenic | chr12 | 113622100 | 113623000 | YES | YES | YES | NO | Hypermethylation |
| 113623000 | | | | | | | | | |
| chr12: 113657800- | Intergenic | chr12 | 113657800 | 113658300 | YES | YES | NO | NO | Hypermethylation |
| 113658300 | | | | | | | | | |
| THEM233 | Promoter | chr12 | 118515500 | 118517500 | YES | YES | NO | YES | Hypermethylation |
| NCOR2 | Intron/Exon | chr12 | 123516200 | 123516800 | YES | YES | YES | NO | Hypermethylation |
| THEM132C | Intron | chr12 | 127416300 | 127416700 | YES | YES | NO | NO | Hypermethylation |
| PTGDR | Promoter | chr14 | 51804000 | 51805200 | YES | YES | NO | NO | Hypermethylation |
| ISL2 | Intron/Exon | chr15 | 74420000 | 74422000 | YES | YES | NO | YES | Hypermethylation |
| chr15: 87750000- | Intergenic | chr15 | 87750000 | 87751000 | YES | YES | NO | NO | Hypermethylation |
| 87751000 | | | | | | | | | |
| chr15: 87753000- | Intergenic | chr15 | 87753000 | 87754100 | YES | YES | NO | NO | Hypermethylation |
| 87754100 | | | | | | | | | |
| NR2F2 | Upstream | chr15 | 94666000 | 94667500 | YES | YES | YES | NO | Hypermethylation |
| chr16: 11234300- | Intergenic | chr16 | 11234300 | 11234900 | YES | YES | NO | NO | Hypermethylation |
| 11234900 | | | | | | | | | |
| SPN | Exon | chr16 | 29582800 | 29583500 | YES | YES | YES | NO | Hypermethylation |
| chr16: 85469900- | Intergenic | chr16 | 85469900 | 85470200 | YES | YES | NO | NO | Hypermethylation |
| 85470200 | | | | | | | | | |
| SLFN11 | Promoter | chr17 | 30725100 | 30725600 | YES | YES | NO | NO | Hypermethylation |
| DLX4 | Upstream | chr17 | 45396800 | 45397800 | YES | YES | NO | YES | Hypermethylation |
| SLC38A10 | Intron | chr17 | 76873800 | 76874300 | YES | YES | YES | NO | Hypermethylation |
| (MGC15523) | | | | | | | | | |
| S1PR4 | Exon | chr19 | 3129900 | 3131100 | YES | YES | YES | YES | Hypermethylation |
| MAP2K2 | Intron | chr19 | 4059700 | 4060300 | YES | YES | YES | NO | Hypermethylation |
| UHRF1 | Intron | chr19 | 4867300 | 4867800 | YES | YES | YES | NO | Hypermethylation |
| DEDD2 | Exon | chr19 | 47395300 | 47395900 | YES | YES | YES | NO | Hypermethylation |
| CDC42EP1 | Exon | chr22 | 36292300 | 36292800 | YES | YES | YES | NO | Hypermethylation |

TABLE 1C
Chromosome 21 differentially methylated regions

| | | | | | Micro- | Epi TYPER | Epi TYPER | Previously | Relative Methyl- |
|---|---|---|---|---|---|---|---|---|---|
| | Gene | | | | array | 8 | 73 | Validated | ation Placenta |
| Region Name | Region | Chrom | Start | End | Analysis | Samples | Samples | Epi TYPER | to Maternal |
| chr21: 9906600- | Intergenic | chr21 | 9906600 | 9906800 | NO | YES | NO | NO | Hypomethylation |
| 9906800 | | | | | | | | | |
| chr21: 9907000- | Intergenic | chr21 | 9907000 | 9907400 | NO | YES | NO | NO | Hypomethylation |
| 9907400 | | | | | | | | | |
| chr21: 9917800- | Intergenic | chr21 | 9917800 | 9918450 | NO | YES | NO | NO | Hypomethylation |
| 9918450 | | | | | | | | | |
| TPTE | Promoter | chr21 | 10010000 | 10015000 | NO | YES | NO | NO | Hypomethylation |
| chr21: 13974500- | Intergenic | chr21 | 13974500 | 13976000 | NO | YES | NO | NO | Hypomethylation |
| 13976000 | | | | | | | | | |
| chr21: 13989500- | Intergenic | chr21 | 13989500 | 13992000 | NO | YES | NO | NO | Hypomethylation |
| 13992000 | | | | | | | | | |
| chr21: 13998500- | Intergenic | chr21 | 13998500 | 14000100 | NO | YES | NO | NO | Hypomethylation |
| 14000100 | | | | | | | | | |
| chr21: 14017000- | Intergenic | chr21 | 14017000 | 14018500 | NO | YES | NO | NO | Hypomethylation |
| 14018500 | | | | | | | | | |
| chr21: 14056400- | Intergenic | chr21 | 14056400 | 14058100 | NO | YES | NO | NO | Hypomethylation |
| 14058100 | | | | | | | | | |
| chr21: 14070250- | Intergenic | chr21 | 14070250 | 14070550 | NO | YES | NO | NO | Hypomethylation |
| 14070550 | | | | | | | | | |
| chr21: 14119800- | Intergenic | chr21 | 14119800 | 14120400 | NO | YES | NO | NO | Hypomethylation |
| 14120400 | | | | | | | | | |
| chr21: 14304800- | Intergenic | chr21 | 14304800 | 14306100 | NO | YES | NO | NO | Hypomethylation |
| 14306100 | | | | | | | | | |
| chr21: 15649340- | Intergenic | chr21 | 15649340 | 15649450 | NO | YES | YES | NO | Hypermethylation |
| 15649450 | | | | | | | | | |
| C21orf34 | Intron | chr21 | 16881500 | 16883000 | NO | YES | NO | NO | Hypomethylation |
| BTG3 | Intron | chr21 | 17905300 | 17905500 | NO | YES | NO | NO | Hypomethylation |
| CHODL | Promoter | chr21 | 18539000 | 18539800 | NO | YES | YES | NO | Hypermethylation |
| NCAM2 | Upstream | chr21 | 21291500 | 21292100 | NO | YES | NO | NO | Hypermethylation |
| chr21: 23574000- | Intergenic | chr21 | 23574000 | 23574600 | NO | YES | NO | NO | Hypomethylation |
| 23574600 | | | | | | | | | |
| chr21: 24366920- | Intergenic | chr21 | 24366920 | 24367060 | NO | YES | NO | NO | Hypomethylation |
| 24367060 | | | | | | | | | |
| chr21: 25656000- | Intergenic | chr21 | 25656000 | 25656900 | NO | YES | NO | NO | Hypomethylation |
| 25656900 | | | | | | | | | |
| MIR155HG | Promoter | chr21 | 25855800 | 25857200 | NO | YES | YES | NO | Hypermethylation |
| CYYR1 | Intron | chr21 | 26830750 | 26830950 | NO | YES | NO | NO | Hypomethylation |
| chr21: 26938800- | Intergenic | chr21 | 26938800 | 26939200 | NO | YES | NO | NO | Hypomethylation |
| 26939200 | | | | | | | | | |
| GRIK1 | Intron | chr21 | 30176500 | 30176750 | NO | YES | NO | NO | Hypomethylation |
| chr21: 30741350- | Intergenic | chr21 | 30741350 | 30741600 | NO | YES | NO | NO | Hypermethylation |
| 30741600 | | | | | | | | | |
| TIAM1 | Intron | chr21 | 31426800 | 31427300 | NO | YES | YES | NO | Hypermethylation |
| TIAM1 | Intron | chr21 | 31475300 | 31475450 | NO | YES | NO | NO | Hypermethylation |
| TIAM1 | Intron | chr21 | 31621050 | 31621350 | NO | YES | YES | NO | Hypermethylation |
| SOD1 | Intron | chr21 | 31955000 | 31955300 | NO | YES | NO | NO | Hypomethylation |
| HUNK | Intron/Exon | chr21 | 32268700 | 32269100 | NO | YES | YES | NO | Hypermethylation |
| chr21: 33272200- | Intergenic | chr21 | 33272200 | 33273300 | NO | YES | NO | NO | Hypomethylation |
| 33273300 | | | | | | | | | |
| OLIG2 | Promoter | chr21 | 33314000 | 33324000 | YES | YES | NO | YES | Hypermethylation |
| OLIG2 | Downstream | chr21 | 33328000 | 33328500 | YES | YES | NO | NO | Hypomethylation |
| RUNX1 | Intron | chr21 | 35185000 | 35186000 | NO | YES | NO | NO | Hypomethylation |
| RUNX1 | Intron | chr21 | 35320300 | 35320400 | NO | YES | NO | NO | Hypermethylation |
| RUNX1 | Intron | chr21 | 35321200 | 35321600 | NO | YES | NO | NO | Hypermethylation |
| RUNX1 | Intron/Exon | chr21 | 35340000 | 35345000 | NO | YES | YES | NO | Hypermethylation |
| chr21: 35499200- | Intergenic | chr21 | 35499200 | 35499700 | NO | YES | YES | NO | Hypermethylation |
| 35499700 | | | | | | | | | |
| chr21: 35822800- | Intergenic | chr21 | 35822800 | 35823500 | NO | YES | YES | NO | Hypermethylation |
| 35823500 | | | | | | | | | |
| CBR1 | Promoter | chr21 | 36364000 | 36364500 | NO | YES | NO | NO | Hypermethylation |
| DOPEY2 | Downstream | chr21 | 36589000 | 36590500 | NO | YES | NO | NO | Hypomethylation |
| SIM2 | Promoter | chr21 | 36988000 | 37005000 | YES | YES | YES | YES | Hypermethylation |
| HLCS | Intron | chr21 | 37274000 | 37275500 | YES | YES | YES | NO | Hypermethylation |
| DSCR6 | Upstream | chr21 | 37300200 | 37300400 | YES | YES | NO | YES | Hypermethylation |
| DSCR3 | Intron | chr21 | 37551000 | 37553000 | YES | YES | YES | NO | Hypermethylation |
| chr21: 37841100- | Intergenic | chr21 | 37841100 | 37841800 | NO | YES | YES | NO | Hypermethylation |
| 37841800 | | | | | | | | | |
| ERG | Intron | chr21 | 38791400 | 38792000 | NO | YES | YES | NO | Hypermethylation |
| chr21: 39278700- | Intergenic | chr21 | 39278700 | 39279800 | NO | YES | YES | NO | Hypermethylation |
| 39279800 | | | | | | | | | |
| C21orf129 | Exon | chr21 | 42006000 | 42006250 | NO | YES | YES | NO | Hypermethylation |
| C2CD2 | Intron | chr21 | 42188900 | 42189500 | NO | YES | YES | NO | Hypermethylation |
| UMODL1 | Upstream | chr21 | 42355500 | 42357500 | NO | YES | YES | NO | Hypermethylation |
| UMODL1/C21orf128 | Intron | chr21 | 42399200 | 42399900 | NO | YES | NO | NO | Hypomethylation |
| ABCG1 | Intron | chr21 | 42528400 | 42528600 | YES | YES | NO | NO | Hypomethylation |
| chr21: 42598300- | Intergenic | chr21 | 42598300 | 42599600 | YES | YES | NO | NO | Hypomethylation |
| 42599600 | | | | | | | | | |
| chr21: 42910000- | Intergenic | chr21 | 42910000 | 42911000 | NO | YES | NO | NO | Hypomethylation |
| 42911000 | | | | | | | | | |
| PDE9A | Upstream | chr21 | 42945500 | 42946000 | NO | YES | NO | NO | Hypomethylation |
| PDE9A | Intron | chr21 | 42961400 | 42962700 | NO | YES | NO | NO | Hypomethylation |
| PDE9A | Intron | chr21 | 42977400 | 42977600 | NO | YES | NO | NO | Hypermethylation |
| PDE9A | Intron/Exon | chr21 | 42978200 | 42979800 | YES | YES | NO | NO | Hypomethylation |
| PDE9A | Intron | chr21 | 43039800 | 43040200 | NO | YES | YES | NO | Hypermethylation |
| chr21: 43130800- | Intergenic | chr21 | 43130800 | 43131500 | NO | YES | NO | NO | Hypomethylation |
| 43131500 | | | | | | | | | |
| U2AF1 | Intron | chr21 | 43395500 | 43395800 | NO | YES | NO | NO | Hypermethylation |
| U2AF1 | Intron | chr21 | 43398000 | 43398450 | NO | YES | YES | NO | Hypermethylation |
| chr21: 43446600- | Intergenic | chr21 | 43446600 | 43447600 | NO | YES | NO | NO | Hypomethylation |
| 43447600 | | | | | | | | | |
| CRYAA | Intron/Exon | chr21 | 43463000 | 43466100 | NO | YES | NO | NO | Hypomethylation |
| chr21: 43545000- | Intergenic | chr21 | 43545000 | 43546000 | YES | YES | NO | NO | Hypomethylation |
| 43546000 | | | | | | | | | |
| chr21: 43606000- | Intergenic | chr21 | 43606000 | 43606500 | NO | YES | NO | NO | Hypomethylation |
| 43606500 | | | | | | | | | |
| chr21: 43643000- | Intergenic | chr21 | 43643000 | 43644300 | YES | YES | YES | YES | Hypermethylation |
| 43644300 | | | | | | | | | |
| C21orf125 | Upstream | chr21 | 43689100 | 43689300 | NO | YES | NO | NO | Hypermethylation |
| C21orf125 | Downstream | chr21 | 43700700 | 43701700 | NO | YES | NO | NO | Hypermethylation |
| HSF2BP | Intron/Exon | chr21 | 43902500 | 43903800 | YES | YES | NO | NO | Hypomethylation |
| AGPAT3 | Intron | chr21 | 44161100 | 44161400 | NO | YES | YES | NO | Hypermethylation |
| chr21: 44446500- | Intergenic | chr21 | 44446500 | 44447500 | NO | YES | NO | NO | Hypomethylation |
| 44447500 | | | | | | | | | |
| TRPM2 | Intron | chr21 | 44614500 | 44615000 | NO | YES | NO | NO | Hypomethylation |
| C21orf29 | Intron | chr21 | 44750400 | 44751000 | NO | YES | NO | NO | Hypomethylation |
| C21orf29 | Intron | chr21 | 44950000 | 44955000 | NO | YES | YES | NO | Hypermethylation |
| ITGB2 | Intron/Exon | chr21 | 45145500 | 45146100 | NO | YES | NO | NO | Hypomethylation |
| POFUT2 | Downstream | chr21 | 45501000 | 45503000 | NO | YES | NO | NO | Hypomethylation |
| chr21: 45571500- | Intergenic | chr21 | 45571500 | 45573700 | NO | YES | NO | NO | Hypomethylation |
| 45573700 | | | | | | | | | |
| chr21: 45609000- | Intergenic | chr21 | 45609000 | 45610600 | NO | YES | NO | NO | Hypomethylation |
| 45610600 | | | | | | | | | |
| COL18A1 | Intron | chr21 | 45670000 | 45677000 | YES | YES | NO | YES | Hypomethylation |
| COL18A1 | Intron/Exon | chr21 | 45700500 | 45702000 | NO | YES | NO | NO | Hypomethylation |
| COL18A1 | Intron/Exon | chr21 | 45753000 | 45755000 | YES | YES | NO | YES | Hypomethylation |
| chr21: 45885000- | Intergenic | chr21 | 45885000 | 45887000 | NO | YES | NO | NO | Hypomethylation |
| 45887000 | | | | | | | | | |
| PCBP3 | Intron | chr21 | 46111000 | 46114000 | NO | YES | NO | NO | Hypomethylation |
| PCBP3 | Intron/Exon | chr21 | 46142000 | 46144500 | NO | YES | NO | NO | Hypomethylation |
| COL6A1 | Intron/Exon | chr21 | 46227000 | 46233000 | NO | YES | NO | NO | Hypomethylation |
| COL6A1 | Intron/Exon | chr21 | 46245000 | 46252000 | NO | YES | NO | NO | Hypomethylation |
| chr21: 46280500- | Intergenic | chr21 | 46280500 | 46283000 | NO | YES | NO | NO | Hypomethylation |
| 46283000 | | | | | | | | | |
| COL6A2 | Intron | chr21 | 46343500 | 46344200 | NO | YES | NO | NO | Hypomethylation |
| COL6A2 | Intron/Exon | chr21 | 46368000 | 46378000 | NO | YES | NO | NO | Hypomethylation |
| C21orf56 | Intron/Exon | chr21 | 46426700 | 46427500 | NO | YES | NO | NO | Hypomethylation |
| C21orf57 | Intron | chr21 | 46541568 | 46541861 | NO | YES | NO | NO | Hypermethylation |
| C21orf57 | Exon | chr21 | 46541872 | 46542346 | NO | YES | NO | NO | Hypermethylation |
| C21orf57 | Downstream | chr21 | 46542319 | 46542665 | NO | YES | NO | NO | Hypermethylation |
| C21orf58 | Intron | chr21 | 46546914 | 46547404 | NO | YES | NO | NO | Hypomethylation |
| PRMT2 | Downstream | chr21 | 46911000 | 46913000 | YES | YES | NO | YES | Hypermethylation |
| ITGB2 | Intron | chr21 | 45170700 | 45171100 | NO | YES | YES | NO | Hypermethylation |

TABLE 2

| GENE | | | | |
|---|---|---|---|---|
| NAME | CHROM | START | END | SNPs |
| chr13 | chr13 | 19773745 | 19774050 | rs7996310; rs12870878 |
| group00016 | | | | |
| chr13 | chr13 | 19290394 | 19290768 | rs11304938 |
| group00005 | | | | |
| CENPJ | chr13 | 24404023 | 24404359 | rs7326661 |
| ATP8A2 | chr13 | 25484475 | 25484614 | rs61947088 |
| PDX1 | chr13 | 27400459 | 27401165 | rs58173592; rs55836809; rs61944011 |
| RB1 | chr13 | 47790983 | 47791646 | rs2804094; rs4151432; rs4151433; rs4151434; rs4151435 |
| PCDH17 | chr13 | 57104856 | 57106841 | rs35287822; rs34642962; rs41292834; rs45500496; |
| | | | | rs45571031; rs41292836; rs28374395; rs41292838 |
| KLHL1 | chr13 | 69579933 | 69580146 | rs3751429 |
| POU4F1 | chr13 | 78079515 | 78081073 | rs11620410; rs35794447; rs2765065 |
| GPC6 | chr13 | 92677402 | 92678666 | rs35689696; rs11839555; rs55695812; rs35259892 |
| SOX21 | chr13 | 94152286 | 94153047 | rs41277652; rs41277654; rs35276096; rs5805873; rs35109406 |
| ZIC2 | chr13 | 99439660 | 99440858 | rs9585309; rs35501321; rs9585310; rs7991728; rs1368511 |
| IRS2 | chr13 | 109232856 | 109235065 | rs61747993; rs1805097; rs9583424; rs35927012; rs1056077; rs1056078; |
| | | | | rs34889228; rs1056080; rs1056081; rs12853546; rs4773092; rs35223808; |
| | | | | rs35894564; rs3742210; rs34412495; rs61962699; rs45545638; rs61743905 |
| chr13 | chr13 | 111808255 | 111808962 | rs930346 |
| group00395 | | | | |
| MCF2L | chr13 | 112724910 | 112725742 | rs35661110; rs2993304; rs1320519; rs7320418; rs58416100 |
| F7 | chr13 | 112799123 | 112799379 | rs2480951; rs2476320 |
| CIDEA | chr18 | 12244327 | 12244696 | rs60132277 |
| chr18 | chr18 | 12901467 | 12901643 | rs34568924; rs8094284; rs8094285 |
| group00091 | | | | |
| C18orf1 | chr18 | 13377536 | 13377654 | rs9957861 |
| KLHL14 | chr18 | 28603978 | 28605183 | rs61737323; rs61737324; rs12960414 |
| CD33L3 | chr18 | 41671477 | 41673011 | rs62095363; rs2919643 |
| ONECUT2 | chr18 | 53254808 | 53259810 | rs35685953; rs61735644; rs8084084; rs35937482; rs35427632; |
| | | | | rs7232930; rs3786486; rs34286480; rs3786485; rs28655657; rs4940717; |
| | | | | rs4940719; rs3786484; rs34040569; rs35542747; rs33946478; rs35848049; |
| | | | | rs7231349; rs7231354; rs34481218; rs12962172; rs3911641 |
| RAX | chr18 | 55086286 | 55086436 | rs58797899; rs45501496 |
| chr18 | chr18 | 57151972 | 57152311 | rs17062547 |
| group00277 | | | | |
| TNFRSF11A | chr18 | 58203013 | 58203282 | rs35114461 |
| NETO1 | chr18 | 68685099 | 68687060 | rs4433898; rs34497518; rs35135773; rs6566677; rs57425572; rs36026929; |
| | | | | rs34666288; rs10627137; rs35943684; rs9964226; rs4892054; rs9964397; |
| | | | | rs4606820; rs12966677; rs8095606 |
| chr18 | chr18 | 70133945 | 70134397 | rs8086706; rs8086587; rs8090367; rs999332; rs17806420; rs58811193 |
| group00304 | | | | |
| TSHZ1 | chr18 | 71128742 | 71128974 | rs61732783; rs3744910; rs1802180 |
| chr18 | chr18 | 74170347 | 74170489 | rs7226678 |
| group00342 | | | | |
| NFATC1 | chr18 | 75385424 | 75386008 | rs28446281; rs56384153; rs4531815; rs3894049 |
| chr18 | chr18 | 75653272 | 75653621 | rs34967079; rs35465647 |
| group00430 | | | | |
| KCNG2 | chr18 | 75760343 | 75760820 | rs3744887; rs3744886 |
| OLIG2 | chr21 | 33317673 | 33321183 | rs2236618; rs11908971; rs9975039; rs6517135; rs2009130; rs1005573; |
| | | | | rs1122807; rs10653491; rs10653077; rs35086972; rs28588289; rs7509766; |
| | | | | rs62216114; rs35561747; rs7509885; rs11547332 |
| OLIG2 | chr21 | 33327593 | 33328334 | rs7276788; rs7275842; rs7275962; rs7276232; rs16990069; |
| | | | | rs13051692; rs56231743; rs35931056 |
| RUNX1 | chr21 | 35180938 | 35185436 | rs2843956; rs55941652; rs56020428; rs56251824; rs13051109; rs13051111; |
| | | | | rs3833348; rs7510136; rs743289; rs5843690; rs33915227; rs11402829; |
| | | | | rs2843723; rs8128138; rs8131386; rs2843957; rs57537540; |
| | | | | rs13048584; rs7281361; rs2843965; rs2843958 |
| SIM2 | chr21 | 36994965 | 36995298 | rs2252821 |
| SIM2 | chr21 | 36999025 | 36999410 | rs58347144; rs737380 |
| DSCAM | chr21 | 41135559 | 41135706 | rs35298822 |
| AIRE | chr21 | 44529935 | 44530388 | rs35110251; rs751032; rs9978641 |
| SUMO3 | chr21 | 45061293 | 45061853 | rs9979741; rs235337; rs7282882 |
| C21orf70 | chr21 | 45202815 | 45202972 | rs61103857; rs9979028; rs881318; rs881317 |
| COL18A1 | chr21 | 45754383 | 45754487 | rs35102708; rs9980939 |
| PRMT2 | chr21 | 46911967 | 46912385 | rs35481242; rs61743122; rs8131044; rs2839379 |
| SIX2 | chr2 | 45081223 | 45082129 | rs62130902 |
| SIX2 | chr2 | 45084851 | 45085711 | rs35417092; rs57340219 |
| SOX14 | chr3 | 138971870 | 138972322 | rs57343003 |
| TLX3 | chr5 | 170674439 | 170676431 | rs11134682; rs35704956; rs2964533; rs35601828 |
| FOXP4 | chr6 | 41623666 | 41624114 | rs12203107; rs1325690 |
| FOXP4 | chr6 | 41636384 | 41636779 | rs56835416 |
| chr7 | chr7 | 12576755 | 12577246 | rs56752985; rs17149965; rs6948573; rs2240572 |
| group00267 | | | | |
| NPY | chr7 | 24290224 | 24291508 | rs2390965; rs2390966; rs2390967; rs2390968; rs3025123; rs16146; rs16145; |
| | | | | rs16144; rs13235842; rs13235935; rs13235938; rs13235940; rs13235944; |
| | | | | rs36083509; rs3025122; rs16143; rs16478; rs16142; rs16141; rs16140; |
| | | | | rs16139; rs2229966; rs1042552; rs5571; rs5572 |
| SHH | chr7 | 155291537 | 155292091 | rs9333622; rs1233554; rs9333620; rs1233555 |
| GLIS3 | chr9 | 4288283 | 4289645 | rs56728573; rs12340657; rs12350099; rs35338539; rs10974444; rs7852293 |
| PRMT8 | chr12 | 3472714 | 3473190 | rs12172776 |
| TBX3 | chr12 | 113609153 | 113609453 | rs60114979 |
| chr12 | chr12 | 118516189 | 118517435 | rs966246; rs17407022; rs970095; rs2711748 |
| group00801 | | | | |
| PAX9 | chr14 | 36201402 | 36202386 | rs17104893; rs12883298; rs17104895; rs35510737; rs12882923; rs12883049; |
| | | | | rs28933970; rs28933972; rs28933971; rs28933373; rs61734510 |
| SIX1 | chr14 | 60178801 | 60179346 | rs761555 |
| ISL2 | chr15 | 74420013 | 74421546 | rs34173230; rs11854453 |
| DLX4 | chr17 | 45397228 | 45397930 | rs62059964; rs57481357; rs56888011; rs17638215; rs59056690; |
| | | | | rs34601685; rs17551082 |
| CBX4 | chr17 | 75428613 | 75431793 | rs1285243; rs35035500; rs12949177; rs3764374; rs62075212; |
| | | | | rs62075213; rs3764373; rs3764372; rs55973291 |
| EDG6 | chr19 | 3129836 | 3130874 | rs34728133; rs34573539; rs3826936; rs34914134; rs61731111; rs34205484 |
| MGC29506 | chr5 | 138757911 | 138758724 | rs11748963; rs7447765; rs35262202 |
| CENTG1 | chr12 | 56406249 | 56407788 | rs61935742; rs12318065; rs238519; rs238520; rs238521; rs808930; |
| | | | | rs2640595; rs2640596; rs2640597; rs2640598; rs34772922 |
| CENTG1 | chr12 | 56416146 | 56418794 | rs11830475; rs34482618; rs2650057; rs2518686; rs12829991 |

TABLE 3

| | RELATIVE METHYLATION | PRC2 |
|---|---|---|
| GENE NAME | PLACENTA TO MATERNAL | TARGET |
| CRYL1 | HYPOMETHYLATION | TRUE |
| IL17D | HYPOMETHYLATION | TRUE |
| GSH1 | HYPERMETHYLATION | TRUE |
| MAB21L1 | HYPERMETHYLATION | TRUE |
| PCDH17 | HYPERMETHYLATION | TRUE |
| KLHL1 | HYPERMETHYLATION | TRUE |
| POU4F1 | HYPERMETHYLATION | TRUE |
| SOX21 | HYPERMETHYLATION | TRUE |
| SHH | HYPERMETHYLATION | TRUE |
| OSR2 | HYPERMETHYLATION | TRUE |
| TBX3 | HYPERMETHYLATION | TRUE |
| PAX9 | HYPERMETHYLATION | TRUE |
| SIX1 | HYPERMETHYLATION | TRUE |
| ISL2 | HYPERMETHYLATION | TRUE |
| DLX4 | HYPERMETHYLATION | TRUE |
| CBX4 | HYPERMETHYLATION | TRUE |
| CENTG1 | HYPOMETHYLATION | TRUE |
| CENTG1 | HYPOMETHYLATION | TRUE |
| ZIC2 | HYPERMETHYLATION | TRUE |
| CIDEA | HYPERMETHYLATION | TRUE |
| KLHL14 | HYPERMETHYLATION | TRUE |
| ONECUT2 | HYPERMETHYLATION | TRUE |
| RAX | HYPERMETHYLATION | TRUE |
| TNFRSF11A | HYPOMETHYLATION | TRUE |
| OLIG2 | HYPERMETHYLATION | TRUE |
| OLIG2 | HYPOMETHYLATION | TRUE |
| SIM2 | HYPERMETHYLATION | TRUE |
| SIM2 | HYPERMETHYLATION | TRUE |
| SIX2 | HYPERMETHYLATION | TRUE |
| SIX2 | HYPERMETHYLATION | TRUE |
| SOX14 | HYPERMETHYLATION | TRUE |
| TLX3 | HYPERMETHYLATION | TRUE |

TABLE 4A

| SEQ | | |
|---|---|---|
| ID | GENE | |
| NO | NAME | SEQUENCE |
| 1 | chr13 | CAGCAGGCGCGCTCCCGGCGAATCTGCCTGAATCGCCGTGAATGCGGTGGGGTGCAGGGCAGGGGCTGGTTTTCTCAGCCGGTCTTGG |
| | group- | CTTTTCTCTTTCTCTCCTGCTCCACCAGCAGCCCCTCCGCGGGTCCCATGGGCTCCGCGCTCAGAACAGCCCGGAACCAGGCGCCGCTC |
| | 00016 | GCCGCTCGCTGGGGGCCACCCGCCTCTCCCCGGAACAGCCTCCCGCGGGCCTCTTGGCCTCGCACTGGCGCCCTCACCCACACATCGT |
| | | CCCTTTATCCGCTCAGACGCTGCAAAGGGCCTTCTGTCTC |
| 2 | CENPJ | GCTTTGGATTTATCCTCATTGGCTAAATCCCTCCTGAAACATGAAACTGAAACAAAGCCCTGAACCCCCTCAGGCTGAAAAGACAAACCCC |
| | | GCCTGAGGCCGGGTCCCGCTCCCCACCTGGAGGGACCCAATTCTGGGCGCCTTCTGGCGACGGTCCCTGCTAGGGACGCTGCGCTCTC |
| | | CGAGTGCGAGTTTTCGCCAAACTGATAAAGCACGCAGAACCGCAATCCCCAAACTAACACTGAACCCGGACCCGCGATCCCCAAACTGAC |
| | | AAGGGACCCGGAACAGCGACCCCCAAACCGACACGGGACTCGGGAACCGCTATCTCCAAAGGGCAGC |
| 3 | ATP8A2 | TTTCCACAACAGGGAGCCAGCATTGAGGCGCCCAGATGGCATCTGCTGGAAATCACGGGCCGCTGGTGAAGCACCACGCCTTACCCGAC |
| | | GTGGGGAGGTGATCCCCCACCTCATCCCACCCCCTTCTGTCTGTCTCCTT |
| 4 | GSH1 | GCTGGACAAGGAGCGCTCACTGTAGCTCTGCTGTGGATTGTGTTGGGGCGAAGAGATGGGTAAGAGGTCAAAGTCGTAGGATTCTGGCG |
| | | ACCGCCTACCAAGGGATTGGGTCCACAGCACAGAGGTCTGATCGCTTCCTTCTCTGCTCTGCCACCTCCAGACAGCAGCTCTAACCAGCT |
| | | GCCCAGCAGCAAGAGGATGCGCACGGCTTTCACCAGCACGCAGCTGCTAGAGCTGGAGCGCGAGTTCGCTTCTAATATGTACCTGTCCC |
| | | GCCTACGTCGCATCGAGATCGCGA |
| 5 | PDX1 | TGCCTGACACTGACCCCAGGCGCAGCCAGGAGGGGCTTTGTGCGGGAGAGGGAGGGGGACCCCAGCTTGCCTGGGGTCCACGGGACT |
| | | CTCTTCTTCCTAGTTCACTTTCTTGCTAAGGCGAAGGTCCTGAGGCAGGACGAGGGCTGAACTGCGCTGCAATCGTCCCCACCTCCAGCG |
| | | AAACCCAGTTGAC |
| 6 | PDX1 | TCGGCGGAGAGACCTCGAGGAGAGTATGGGGAAAGGAATGAATGCTGCGGAGCGCCCCTCTGGGCTCCACCCAAGCCTCGGAGGCGG |
| | | GACGGTGGGCTCCGTCCCGACCCCTTAGGCAGCTGGACCGATACCTCCTGGATCAGACCCCACAGGAAGACTCGCGTGGGGCCCGATA |
| | | TGTGTACTTCAAACTCTGAGCGGCCACCCTCAGCCAACTGGCCAGTGGATGCGAATCGTGGGCCCTGAGGGGCGAGGGCGCTCGGAAC |
| | | TGCATGCCTGTGCACGGTGCCGGGCTCTCCAGAGTGAGGGGGCCGTAAGGAGATCTCCAAGGAAGCCGAAAAAAGCAGCCAGTTGGGC |
| | | TTCGGGAAAGACTTTTCTGCAAAGGAAGTGATCTGGTCCCAGAACTCCAGGGTTGACCCCAGTACCTGACTTCTCCGGGAGCTGTCAGCT |
| | | CTCCTCTGTTCTTCGGGCTTGGCGCGCTCCTTTCATAATGGACAGACACCAGTGGCCTTCAAAAGGTCTGGGGTGGGGGAACGGAGGAA |
| | | GTGGCCTTGGGTGCAGAGGAAGAGCAGAGCTCCTGCCAAAGCTGAACGCAGTTAGCCCTACCCAAGTGCGCGCTGGCTCGGCATATGC |
| | | GCTCCAGAGCCGGCAGGACAGCCCGGCCCTGCTCACCCCGAGGAGAAATCCAACAGCGCAGCCTCCTGCACCTCCTTGCCCCAGAGAC |
| 7 | MAB21 | AGATCCCGGTGCATTTAAAGGCCGGCGTGATCTGCACCACGTACCTATCTCGGATTCTCAGTTTCACTTCGCTGGTGTCTGCCACCATCTT |
| | L1 | TACCACATCCCGGTAGCTACATTTGTCTACCGCTTGAGCCACCAGCGTCTGAAACCTGGACCGGATTTTGCGCGCCGAGAGGTAGCCGG |
| | | AGGCGGTAATGAATTCCACCCAGAGGGACATGCTCCTCTTGCGCCCGTCGCTCAACTTCAGCACCGCGCAGCCGGGCAGTGAGCCATCG |
| | | TCCACGAAGTTGAACACCCCCATTTGGTTGAGATAAAGCACCACTTCAAATTCGGT |
| 8 | RB1 | ACTATGCCTTGAGGGTCAAAACGTCTGGATTTCCTGATCGATGCTGTCGTCGCTGTCCACGGAGCTACTGTCGCCGTCAGAGCGGGAAG |
| | | GCACGTTCAGGGAGTAGAAGCGTGGGCTTGCAGAAAGGGACCTGTTGCTGCCTTACATGGGGGCCGGCAGGGTAGTCTTGGAAATGCC |
| | | CAAGATTGCTTCCGCGCGCGTCAGTTCAGCGGACGTGTCTGCCTGGCACGAGGACCGTTCTACAAACTCGTTCCTGGAAGCCGGGCTCG |
| | | CTGGAGGCGGAGCTTTGGTTTCCTTCGGGAGCTTGTGGGGAATGGTCAGCGTCTAGGCACCCCGGGCAAGGGTCTGTGGCCTTGGTGG |
| | | CCACTGGCTTCCTCTAGCTGGGTGTTTTCCTGTGGGTCTCGCGCAAGGCATTTTTTGTGGCGCTGCTTGTGCTGTGTGCGGGGTCAGGC |
| | | GTCCTCTCTCCTCCCGGCGCTGGGCCCTCTGGGGCAGGTCCCCGTTGGCCTCCTTGCGTGTTTGCCGCAGCTAGTACACCTGGATGGCC |
| | | TCCTCAGTGCCGTCGTTGCTGCTGGAGTCTGACGCCTCGGGCGCCTGCGCCGCACTTGTGACTTGCTTTCCCCTTCTCAGGGCGCCAGC |
| | | GCTCCTCTTGACCCCGCTTTTATTCTGTGGTGCTTCTGAAG |
| 9 | PCDH17 | GCAAGTCGGGTAGCTACCGGGTGCTGGAGAACTCCGCACCGCACCTGCTGGACGTGGACGCAGACAGCGGGCTCCTCTACACCAAGCA |
| | | GCGCATCGACCGCGAGTCCCTGTGCCGCCACAATGCCAAGTGCCAGCTGTCCCTCGAGGTGTTCGCCAACGACAAGGAGATCTGCATGA |
| | | TCAAGGTAGAGATCCAGGACATCAACGACAACGCGCCCTCCTTCTCCTCGGACCAGATCGAAATGGACATCTCGGAGAACGCTGCTCCG |
| | | GGCACCCGCTTCCCCCTCACCAGCGCACATGACCCCGACGCCGGCGAGAATGGGCTCCGCACCTACCTGCTCACGCGCGACGATCACG |
| | | GCCTCTTTGGACTGGACGTTAAGTCCCGCGGCGACGGCACCAAGTTCCCAGAACTGGTCATCCAGAAGGCTCTGGACCGCGAGCAACAG |
| | | AATCACCATACGCTCGTGCTGACTGCCCTGGACGGTGGCGAGCCTCCACGTTCCGCCACCGTACAGATCAACGTGAAGGTGATTGACTC |
| | | CAACGACAACAGCCCGGTCTTCGAGGCGCCATCCTACTTGGTGGAACTGCCCGAGAACGCTCCGCTGGGTACAGTGGTCATCGATCTGA |
| | | ACGCCACCGACGCCGATGAAGGTCCCAATGGTGAAGTGCTCTACTCTTTCAGCAGCTACGTGCCTGACCGCGTGCGGGAGCTCTTCTCC |
| | | ATCGACCCCAAGACCGGCCTAATCCGTGTGAAGGGCAATCTGGACTATGAGGAAAACGGGATGCTGGAGATTGACGTGCAGGCCCGAGA |
| | | CCTGGGGCCTAACCCTATCCCAGCCCACTGCAAAGTCACGGTCAAGCTCATCGACCGCAACGACAATGCGCCGTCCATCGGTTTCGTCTC |
| | | CGTGCGCCAGGGGGCGCTGAGCGAGGCCGCCCCTCCCGGCACCGTCATCGCCCTGGTGCGGGTCACTGACCGGGACTCTGGCAAGAA |
| | | CGGACAGCTGCAGTGTCGGGTCCTAGGCGGAGGAGGGACGGGCGGCGGCGGGGGCCTGGGCGGGCCCGGGGGTTCCGTCCCCTTCA |
| | | AGCTTGAGGAGAACTACGACAACTTCTACACGGTGGTGACTGACCGCCCGCTGGACCGCGAGACACAAGACGAGTACAACGTGACCATC |
| | | GTGGCGCGGGACGGGGGCTCTCCTCCCCTCAACTCCACCAAGTCGTTCGCGATCAAGATTCTAGACGAGAACGACAACCCGCCTCGGTT |
| | | CACCAAAGGGCTCTACGTGCTTCAGGTGCACGAGAACAACATCCCGGGAGAGTACCTGGGCTCTGTGCTCGCCCAGGATCCCGACCTGG |
| | | GCCAGAACGGCACCGTATCCTACTCTATCCTGCCCTCGCACATCGGCGACGTGTCTATCTACACCTATGTGTCTGTGAATCCCACGAACG |
| | | GGGCCATCTACGCCCTGCGCTCCTTTAACTTCGAGCAGACCAAGGCTTTTGAGTTCAAGGTGCTTGCTAAGGACTCGGGGGCGCCCGCG |
| | | CACTTGGAGAGCAACGCCACGGTGAGGGTGACAGTGCTAGACGTGAATGACAACGCGCCAGTGATCGTGCTCCCCACGCTGCAGAACGA |
| | | CACCGCGGAGCTGCAGGTGCCGCGCAACGCTGGCCTGGGCTATCTGGTGAGCACTGTGCGCGCCCTAGACAGCGACTTCGGCGAGAGC |
| | | GGGCGTCTCACCTACGAGATCGTGGACGGCAACGACGACCACCTGTTTGAGATCGACCCGTCCAGCGGCGAGATCCGCACGCTGCACC |
| | | CTTTCTGGGAGGACGTGACGCCCGTGGTGGAGCTGGTGGTGAAGGTGACCGACCACGGCAAGCCTACCCTGTCCGCAGTGGCCAAGCT |
| | | CATCATCCGCTCGGTGAGCGGATCCCTTCCCGAGGGGGTACCACGGGTGAATGGCGAGCAGCACCACTGGGACATGTCGCTGCCGCTC |
| | | ATCGTGACTCTGAGCACTATCTCCATCATCCTCCTA |
| 10 | KLHL1 | ATGCGCCCTCTGCACCCCTAGAGCCAGAAGACGCTAGGTGGGCTGCGCGCTCTGCCAGGCGAAGGCTGGAGCGCAGACGGCAAAGCC |
| | | GCGCGTTTCAGCCGTGGTCGGGTCCGCAGGACCTGGGCGTGGGGACACCACCAGGCAGGAGCAGAGGCAGGACTGGGACGCCAAAAG |
| | | CTGAGAATCCTCGATGCCCGCGCGAGAGCCCCGTGTTAT |
| 11 | POU4F1 | TTCTGGAAACCGGGCCCCACTTGCAGGCCCGGCCACCTTGGGTTCTGGTGGCCGAAGCCGGAGCTGTGTTTCTCGCAGACTCGGGGAG |
| | | CTACATTGTGCGTAGGCAATTGTTTAGTTTGAAAGGAGGCACATTTCACCACGCAGCCAGCGCCCTGCATGCAGGAGAAGCCCCCAGGG |
| | | CCCAGGGTCGGCTGGCTTTAGAGGCCACTTAGGTTGTTTTAAGCACATGTGAAAGGGCAGACAGCAGGGGAGCAGGATATGGGTAAGAT |
| | | CTTCGGGTCTCAGAACAGGGGCTGCCCTTGGGCTGTCCCGGCGCCCTGGGCTCTGACACTGAAGGGTGGAATGGAGGAAGGAATGGAG |
| | | AAAGGACGGTGGAACTTTCGCTTCCCCTCTGGGCCGCCTTCCCAGGGTCATGCCTGAGCTGCTTTGATCCCAGTGTCGCGCATCTTGGTC |
| | | CGCTACCTCCCAGGCGATAGCTACTGGGCTCCTCGCTGGCCTCACTGGGGGCCATCCCGGGCAGTGGCCTGCCCTCCGAGGCCCGCGG |
| | | GACCCAGCCCAGAGCTGAGGTTGGAGTTCTCCGGGCCACGTTCCGGGTCGCTTAGGCTCGGAGATTTCCCGGAGACCGTCGTCCTCCCT |
| | | TTCTGCTTGGCACTGCGGAGCTCCCTCGGCCTCTCTCCTCCTCTGGTCCCTAAGGCCCGGAGTGGTTGGCGGTACTGGGGCCCGTCGTC |
| | | ATCTCTGCTTCTAAGGCATTCAGACTGGGCTCCAGCTGGGACCGGCAGAGGAGGTTCTCAAGGAAACTGGTGGGAAATATAGTTTTCTTT |
| | | CGTCTGGTCGTTTAATTTAAATGCAACTTCCCTTGGGGACATTTTCCTGGACGTTAACCAGACCACCTTGAGATGTCGTTGATGACCTAGA |
| | | GACCCAGATGATGCGTCCCAGGAAAGTTCACTGCTGACTATTGTCACTCTTGGCGTTATATCTATAGATATAGACCTATGTACATATCTCC |
| | | ACCCTGATCTCTCCGTGGACATGAAACCCACCTACCTTGTGAAAGCCCTACGGGTGACACATGACTACTACGTCTCTGTCCCAACAGGGGC |
| | | TGGGCCTCCCCTGCCTAATAGTTGCCAGGAGTTTCGCAGCCCAAGTGAATAATGTCTTATGGCTGAACGTGGCCAAGGACTCCTGTGATT |
| | | TAGGTCCCAGGAGGAGCAGAGACGTCCCCGCCCCGCCTGGGCCCTGCCGCATTCAAAGCTGGAAGAAGGCGCTGATCAGAGAAGGGGC |
| | | TTCCAGGTCCTGGGTTAGAACAACAACAAACAAACGAAACTCCACAACAGACACGCCTGCCCATGACCCCACGCAAGGACATAGGAAGTT |
| | | CTGTCGCCTTCCTGCTCCGCGGATAGCCGCCTGCCGTCTGCTGCCACCAGAACGCACGGACGCTCGGGGTGGAGGTAGTCAATGGGCA |
| | | GCAGGGGACCCCCAGCCCCCACAAGCGCGGCTCCGAGGACCTGGAAGCGGGTGCCTGTCGCTCTCCGCAGGCTCCGCTCTGCCTCCA |
| | | GGAGCAAGATCCCCAAAAGGGTCTGGAAGCTGTGGAGAAAAC |
| 12 | GPC6 | TTTTTTAAACACTTCTTTTCCTTCTCTTCCTCGTTTTGATTGCACCGTTTCCATCTGGGGGCTAGAGGAGCAAGGCAGCAGCCTTCCCAGC |
| | | CAGCCCTTGTTGGCTTGCCATCGTCCATCTGGCTTATAAAAGTTTGCTGAGCGCAGTCCAGAGGGCTGCGCTGCTCGTCCCCTCGGCTGGC |
| | | AGAAGGGGGTGACGCTGGGCAGCGGCGAGGAGCGCGCCGCTGCCTCTGGCGGGCTTTCGGCTTGAGGGGCAAGGTGAAGAGCGCACC |
| | | GGCCGTGGGGTTTACCGAGCTGGATTTGTATGTTGCACCATGCCTTCTTGGATCGGGGCTGTGATTCTTCCCCTCTTGGGGCTGCTGCTC |
| | | TCCCTCCCCGCCGGGGCGGATGTGAAGGCTCGGAGCTGCGGAGAGGTCCGCCAGGCGTACGGTGCCAAGGGATTCAGCCTGGCGGAC |
| | | ATCCCCTACCAGGAGATCGCAGGTAAGCGCGGGCGCGCTGCAGGGGCAGGCTGCAGCCCTCGGCTGCCGCACGTCCCACTGGCCGCC |
| | | CGGCGTCCCCTTCCTTCCCCCTGTTGCTGAGTTGGTGCTCACTTTCTGCCACCGCTATGGGACTCCGCGTCTCCGTGTTGGGCGGCGGA |
| | | TGCTCCTGCGGCTTCTTCGGCGGGGGAAGGTGTGCGTCTCCGCCGCCTCATTGTGTGCACACGCGGGAGCACCCTGGCTCCCGCCTCC |
| | | CGCTGCTCTCGCGCCCTTCTACCCCTTAGTTGATGGCTCAGGCCCGGCTGGCCAGGGAGCCCGGGTCACTCCGGGGCGGCTGCAAGGC |
| | | GCAGACGGAGAGCCGAGCCGGGCGCTCACTCCGCGTTCTGGTTCGGGCAAACTTGGAAGAACTGCGACCGCAGTTTGCCCAGCGCCAC |
| | | AGTCTGAGTGGCGCCTTCTCCACTCCCGCCCTTGCGCCGGCAGGGGCGGTGGAGAGACGCGGAGGGCTCCCCCAGCCCCTCTCTCCCC |
| | | TATCCGTCCTTCGGGCGACAGAGCGCCCGGCGCTCGGGCCGGGGGCGGGCAAGGCTGGGAGGGACCCTCGCCGGGGACCTGGCCTC |
| | | TGGACGCCGGCGTTTCAAGGCTGGTTTGGGGACTTCACGGGCTGCCTGTTTCAGATGTGGGGCGGGCTTTCCCGTTAGGGTTCCTCAGT |
| | | GCTTCCCCAGTTGCTGTTGGCCACTCAGGGCCCGGGGACACCCTGCCACCCGGTCTGGAGCCGGCCTCGTCTGCCAGCGAACAGCCAA |
| | | CTTTAGCGGGTGGCTCAGCTGGGGATT |
| 13 | SOX21 | CACTCAGTGTGTGCATATGAGAGCGGAGAGACAGCGACCTGGAGGCCATGGGTGGGGGCGGGTGGTGAAGCTGCCGAAGCCTACACAT |
| | | ACACTTAGCTTTGACACTTCTCGTAGGTTCCAAAGACGAAGACACGGTGGCTTCAGGGAGACAAGTCGCAAGGGCGACTTTTCCAAGCGG |
| | | GAGATGGTGAAGTCTTTGGACGTGTAGTGGGTAGGTGATGATCCCCGCAGCCGCCTGTAGGCCCGCAGACTTCAGAAAACAAGGGCCTT |
| | | CTGTGAGCGCTGTGTCCTCCCCGGAATCCGCGGCTTAACACATTCTTTCCAGCTGCGGGGCCAGGATCTCCACCCCGCGCATCCGTGGA |
| | | CACACTTAGGGTCGCCTTTGTTTTGCGCAGTGATTCAAGTTGGGTAACCCTTGCTCAACACTTGGGAAATGGGGAGAATCTCCCCCACCC |
| | | GCAACCTCCCGCACCCCAGGTTCCCAAAATCTGAATCTGTATCCTAGAGTGGAGGCAGCGTCTAGAAAGCAAAGAAACGGTGTCCAAAGA |
| | | CCCCGGAGAGTTGAGTGAGCGCAGATCCGTGACGCCTGCGGTACGCTAGGGCATCCAGGCTAGGGTGTGTGTGTGCGGGTCGGGGGG |
| | | CGCACAGAGACCGCGCTGGTTTAGGTGGACCCGCAGTCCCGCCCGCATCTGGAACGAGCTGCTTCGCAGTTCCGGCTCCCGGCGCCCC |
| | | AGAGAAGTTCGGGGAGCGGTGAGCCTAGCCGCCGCGCGCTCATGTTTATT |
| 14 | ZIC2 | AGTCACTCCAGGATCAGAGGCCGCGTCGGTTCTGCTTGGGGCATGGGCAGAGGGAGGCTGCTGGGGCCAAGCCCCGGCTGGACGCGA |
| | | GGGAAGAAACTCGTCCCAGGACCCGCACGCCCATACCTGGCTGTCCCAGAGCTCTTCCCTAGGCCGGCACCTTCGCTCTTCCTCTTCCC |
| | | CACCCCCTAGCCCTTTGTCTCTTTTTCAGACGGATGTTTTCAGTCTCAAGTGGTTTTATTTTCCGCACAAAACCCTGAGATCAAGGGCAGA |
| | | TCACAGACTGTACCGGAGGCTCGGGTTTCCCTGGACTCTGTGCTGTTCTGCGTCCCAGGGTTGGCTAGGAAGGAAGGCCTGGGCCGGC |
| | | GAGGTGACGGGTCTCCCGCCCAGGTCGGCAGGACGGGGGGAGGTGTGTCCCGGTAGGTCCCTGGTGAGCTCACCCGTGGCATCGGGG |
| | | ACCCGCGGGAACCCACCGGGCGCCCACTAGAGACTCGGGTCCTACCCTCCCCCACACTACTCCACCGAAATGATCGGAAGGGCGCGCTA |
| | | GGCCTGCTTCCAAGGGCTCAGTGATAAAGGCCTCAAAATCACACTCCATCAAGACTTGGTTGAAGCTTTGGGTAGGTTTGTTGTTGTTGTT |
| | | GTTGTTGTTTGTTTGTTTGTTTTAGCAGACACGTCCTGGAAAGAGGTCCTCAGAACCCAAAGGTTCAATAATGATTTGTGGATGGATTGAT |
| | | TATAGTCTGATATCGCTCTGGTTCCACAGAAACCCGGAGCTCCTTGGCCCACTGTTACCCCAGCAGACCTAAATGGACGGTTTCTGTTTTT |
| | | CACTGGCAGCTCAGAACTGGACCGGAAGAAGTTCCCCTCCACTTCCCCCCTCCCGACACCAGATCATTGCTGGGTTTTTATTTTCGGGGG |
| | | AAAAACAACAACAACAACAACAAAAAAAACACTAGGTCCTTCCAGACTGGATCAGGTGATCGGGCAAAAACCCTCAGGCTAGTCCGGCTG |
| | | GGTGCCCGAGCATGAAAAGGCCTCCGTGGCCGTTTGAACAGGGTGTTGCAAATGAGAACTTTTGTAAGCCATAACCAGGGCATCCTGAG |
| | | GGTCTGAGTTCACGGTCAAGGCTGTGGGCTACTAGGTCCAGCGAGTCCAGGCCTCGCCCCGCCCCCGAGCTGCCACAGCCAAGATCTTC |
| | | GGCAGGGAATTCGAGACCAGGGTCCTCCCACTCCT |
| 15 | chr13 | TTTCGTGCCGCTGTTTTCAATGCGCTAACGAGGCACGTTATTCTTAGCCGCGTCCGGGAGGGGATCACATTCCTGCGCAGTTGCGCTGCT |
| | group- | GGCGGAAGTGACTTGTTTTCTAACGACCCTCGTGACAGCCAGAGAATGTCCGTTTCTCGGAGCGCAGCACAGCCTGTCCCATCGAGAAG |
| | 00385 | CCTCGGGTGAGGGGCCCGGTGGGCGCCCGGAGGCCGCTGGAGGGCTGTGGGAGGGACGGTGGCTCCCCACTCCCGTGGCGAAGGGC |
| | | AGGCAAACCAGAAGCCTCTTTTGAGAGCCGTTTGGGATTGAGACGAGTAAGCCACAGCGAGTGGTTAGAAGTAGGTTAGGAAGAAGGGG |
| | | AGGTAAGAAAGCCGAGTAGGGTT |
| 16 | chr13 | GTTCGGTGGACAAGGGGGCAGCGCCCACAGCAAGCCGGAAAGAGGGAGGCGCGGGGCCGCGCTTGGGGCCTGCCGCTGCACGCCAG |
| | group- | CCTGGGCAAAGAGCTGCCACCTTCTGCGGGCGAAGCGGGTCGGGACGCAGGACGGCAGCGGGGCTGGAGGCAGCTACGTGGGTCCAC |
| | 00390 | ACCCCCATGCCCTGCAAGGCTCCTTGGCCCTGCTTCTCCTCTGTCTCGGCGGGAGAGGAGCAGCCTCGGTTTTACAGAATTTC |
| 17 | chr13 | TGTGCCATTTAGTGAGAGGTGTTTTGGGCAAAGAATCAATTTAACTGTGACTGACCGACGGGCTTGACTGTATTAATTCTGCTACCGAAAA |
| | group- | AAAAAAAAAAAAAAAAGCAATGAGCCGCAAGCCTTGGACTCGCAGAGCTGCCGGTGCCCGTCCGAGAGCCCCACCAGCGCGGCTCACGC |
| | 00391 | CTCAGTCTC |
| 18 | chr13 | AGAGTCCCAGTTCTGCAGGCCGCTCCAGGGCTAGGGGTAGAGATGGTGGCAGGTGGTGCGTCAACTCTCTAGGGAAGAGGAACTTGCAT |
| | group- | TACAAAGACTTGTCTTTCTGAGCTGAAGTCAAAACGGGGGCGTCAAGCGCGCTCCGTTTGGCGGCGGTGGAGGGGCCGCGCGCCCGCG |
| | 00395 | CTGTCCCAGCCGGAGCTGCCCTGGCTGGTGATTGGAGGTTTAACGTCCGGAATTCAGGCGCTTCTGCAGCTCAGATTTGCCGGCCAAGGGG |
| | | CCTCAGTTGCAACTTTTCAAAATGGTGTTTCTGGAAAATAACAAATTCAGACTCAACTGGTGACAGCTTTTGGCTATAGAGAATGAAACTG |
| | | CTTCCCTTTGGCGGTGGAACTCTTAAACTTCGAAGAGTGAAAGAATACAATGAAATAAAATGCCATAAGATCACTGGATTTTTCAGAAAAA |
| | | GGAAGACCCCAAATTACTCCCAAAATGAGGCTTTGTAAATTCTTGTTAAAAATCTTTAAATCTCGAATTTCCCCCTACAACATCTGATGAG |
| | | TGCTTTAAGAGCAAACGAGCAAATCCCACCTCGAGAATCAACAAACCCAAGCTCTGGCCAAGGCTCTCCCCGCGTTTTCTTCTCGTGACCT |
| | | GGGGAATGTCCCGCCCCATCGCTCACCTGGCTCTTGTCATCTCGCTCATCTTGAAGTGACCCGTGGACAATGCTG |
| 19 | chr13 | AGCTGCCCTCTGTGGCCATGAGCGGGTGTCCAGCCCCTTCCAAGGCTGCACCGGGGAGACGCTGGTTTTCTGCTCGCTGTGACCGAACA |
| | group- | AAGCCCCTAAGAGTCAGTGCGCGGAACAGAAGAGCCGGACCCCGACGGGCCGAGTCCCAACGTGAGGCACCCGGCAGAGAAAACACGT |
| | 00399 | TCACG |
| 20 | PROZ | CCTCGGCAGCACCGGCATGGCTGGAGGCCAGTACGGCCAGGTGTGGCGGGAGGGAGCGCCGTCTGGCTTGGGTCGTCCATCCTGACA |
| | | GGACGCTGCAAGGGCAGGAGCCCCGCGCCCCGTGTCCTGCGCCCCCGCTCGAGGACAAGCCCCAGCCGCCGGTCTCCGCTGGGTTCC |
| | | GACAG |
| 21 | CIDEA | CTTTAAGAGGCTGTGCAGGCAGACAGACCTCCAGGCCCGCTAGGGGATCCGCGCCATGGAGGCCGCCCGGGACTATGCAGGAGCCCTC |
| | | ATCAGGCGAGTGCCCCGCGTCCCCCTGATTGCCGTGCGCTTCCAATCGCCTTGCGTTCGGTGGCCTCATATTCCCCTGTGCGCCTCTAGT |
| | | ACCGTACCCCGCTCCCTTCAGCCCCCTGCTCCCCGCATTCTCTTGCGCTCCGCGACCCCGCGCACACACCCATCCGCCCCACTGGTGCC |
| | | CAAGCCGTCCAGCCGCGCCCGCGGGCAGAGCCCAATCCCGTCCCGCGCCTCCTCACCCTCTTGCAGCTGGGCACAGGTACCAGGTGTG |
| | | GCTCTTGCGAGGTG |
| 22 | chr18 | AGACTTGCAGAACTCGGGCCCCCTGGAGGAGACCTAACCGCCACGGTCTTGGGGAGGTTCCGGAGGGCCTCGGTTGTCTGCACTCCCA |
| | group- | ACACCAAGAAACCCCTGAGACGCGAAGCTGCCAGCGTGCTGCCCTCAGAGCAGGGCGACGCAAAGCCAGCGGACCCCGGGGTGGCGG |
| | 00091 | G |
| 23 | chr18 | TGCTCGGCTGGGGGGCTCGCTCCGCACTTTCGGTGCCAGAAAATGCCCAGAGGAGCGGGGCGGCCCCAGAGCCTCCTTTCGGGGCGC |
| | group- | GAGGCCCGGCGCGTGTGTACGGAGTCCAGTCCCCCCAGGGAGTGGGGTGCCCGCACCTTCCCCTCCGCGCTCGGAGCCAC |
| | 00094 | |
| 24 | KLHL14 | TCTTGCACACCTGCTTGTAGTTCTGCACCGAGATCTGGTCGTTGAGGAACTGCACGCAGAGCTTGGTGACCTGGGGGATGTGCAGGATCT |
| | | TGCTGACCGACAGCACCTCCTCCACCGTGTCCAGGGACAGGGTCACGTTGGCCGTGTAGAGGTACTCGAGCACCAGGCGCAGCCCGAT |
| | | GGACGAGCAGCCCTGCAGCACCAGGTTGTTGATGGCCCGGGGGCTGGTCAGCAGCTTGTCGTCGGGGGAGGAAGAAGGAGTCCCGGG |
| | | CTCCTCCTGCGGCGGCGGCTGCTGCTGCTGTGACGGCTGCTGCTGCGGCGGCTGCTGCTGGTCCTTGGGGGCCCCCAGGCCGTCCTG |
| | | GCCGCCGACCCCTCCCCCGAGAGGGGGGTGGCTGGAGAAGAGCGATCGGAAGTACTGCGAGCAGGAGGCCAGCACGGCCTTGTGGCA |
| | | ATGGAACTGCTGGCCCTGGGCCGTCAGGGTCACGTCGCAAAACAGCTGCTTCCTCCACAGCAGGTTGAGGCCGTGCAGCAGGTTGTCGC |
| | | TGTGGCTGGGGTCGAAGGTGGAGGTCCTGTCCCCGGATCTGGACATGGCGAGCTGACTCGGTGCACCTGGCTTTAAACCCTCCTCCAAC |
| | | CTGGCAGACAGGGGTGGGGGATGGGAGGGAGGGGAGCAGGGTGGTGGAGCGGGTGGGGTGTGGTCGGGGTGGGGAAGGGTGTGGA |
| | | GGGGAGGGGAGGGCGAAGAACAAGAATCAAGGCTCAGCTTGACTCCCTCCTGGCGCGCTCCGGACCCCGACCCTAGGAGGAAAGTCCG |
| | | AAGACGCTGGATCCGTGAGCGCCACCAGAAGGGCCCTGTCTGGGGTCCCGGCGCCGGTTCTGCGCCCTGCGGCTCCTCTCGCCACCTC |
| | | CCACACACTTCGTCCCTCACTTTCCTAAAACCAACCACCTCAGCTCGGCTGTTGGCAGCAACAGCAGTGGCAGCAGCGACGGCAAAGTG |
| | | GCGGCTGAGGCCGAGGCACCTCGTGGGCTCGTGTCCATGCCGGGCCAGATGAAGGGAAAGGCCGGGAAGTGGGGAGCCGGGGGTGC |
| | | CCTGAAAGCTCAGAGGCGACCGACGGCGAAGGTTCCAGGTCAACTTGTGCCCGAAGCTTTGCTTTTCGCAGTTGGCCCAGTTTGGGGGA |
| | | GGGGGTAGGAACAGGGGCCCGACCAGCGTGCGGGGTGTGCGAATCTTAGCTCTCCAAAAGCTG |
| 25 | ST8SIA3 | CCTCTGTGTTAGTGCCCTCGGGAATTTGGTTGATGGGGTGTTTG |
| 26 | ONECUT2 | TGATGTCGCACCTGAACGGCCTGCACCACCCGGGCCACACTCAGTCTCACGGGCCGGTGCTGGCACCCAGTCGCGAGCGGCCACCCTC |
| | | GTCCTCATCGGGCTCGCAGGTGGCCACGTCGGGCCAGCTGGAAGAAATCAACACCAAAGAGGTGGCCCAGCGCATCACAGCGGAGCTG |
| | | AAGCGCTACAGTATCCCCCAGGCGATCTTTGCGCAGAGGGTGCTGTGCCGGTCTCAGGGGACTCTCTCCGACCTGCTCCGGAATCCAAA |
| | | ACCGTGGAGTAAACTCAAATCTGGCAGGGAGACCTTCCGCAGGATGTGGAAGTGGCTTCAGGAGCCCGAGTTCCAGCGCATGTCCGCCTT |
| | | ACGCCTGGCAGGTAAGGCCGGGGCTAGCCAGGGGCCAGGCTGCTGGGAAGAGGGCTCCGGGTCCGGTGCTTGTGGCCCAAGTCTGCGCGC |
| | | CGAGTCACTTCTCTTGATTCTTTCCTTCTCTTTCCTATACACGTCCTCTTTCTTCTCGTTTTTATTTCTTCTTCCATTTTCTCTTTCTCTT |
| | | CCGCTCTTCCCCTACTTTCCCTTCTCCCTTTTCTTTTTCTTTCTTACTCTCTCCTTGTCCCTGAGCTTTCATTGACCGACCCCCCCCCATT |
| | | TCATTCGCCCTCCCCTCAATGTGCCAACCTTTGCCCTATTTCCGATCTTCCCAGGTACTGGGAGGCGGGATGGGGGTGTGCGTTTTCCTCT |
| | | AGGAGCCCTGTCTTTCCAAGACCCACAGAAACCAGGACCTGCCCTTATTCAAAACCCCATGCACTTCAAGTCTCTTTTAGACAACACATTT |
| | | CAATTTTCCGGGCTGACTAGTCTCCCTGTGCAGAGGCAGTTGAGAGGCTTTGCTCTGCAGAGGGAAAAGAGCTCTCTACTCTCCCACCCAC |
| | | CATATAGGCAAACTTATTTGGTCATTGGCTGAAGGCACAGCCTTGCCCCCGCGGGGAACCGGCGGCCAGGATACAACAGCGCTCCTGGA |
| | | GCCCATCTCTGGCCTTGGCGTTGGCGCAGGGACTTTCTGACCGGGCTTGAGGGGCTCGGGCCAGCTCCAATGTCACTACCTACAGCGAG |
| | | GGCAGGGTGTAAGGTTGAGAAGGTCACATTCACCGCTTTGGGAGGACGTGGGAGAAGAGACTGAGGTGGAAAGCGCTTTGCCTTGCTCA |
| | | CCGGCCGTCCTTGCCCCGGTCCCAGCGTTTGCTGGGATTTGCCAGGATTTGCCGGGGCTCCGGGAGACCCTGAGCACTCGCAGGAAGA |
| | | GGTGCTGAGAAATTAAAAATTCAGGTTAGTTAATGCATCCCTGCCGCCGGCTGCAGGCTCCGCCTTTGCATTAAGCGGGCGCTGATTGTG |
| | | CGCGCCTGGCGACCGCGGGGAGGACTGGCGGCCCGCGGGAGGGGACGGGTAGAGGCGCGGGTTACATTGTTCTGGAGCCGGCTCGG |
| | | CTCTTTGTGCCTCCTCTAGCGGCCAAGCTGCGAGGTACAGCCCTCTATTGTTCTAGGAGCACAGAAACCTCCTGTGTGGGCGGCGGGTG |
| | | CGCGAGCTAGAGGGAAAGATGCAGTAGTTACTGCGACTGGCACGCAGTTGCGCGCTTTTGTGCGCACGGACCCCGCGCGGTGTGCGTG |
| | | GCGACTGCGCTGCCCCTAGGAGCAAGCCACGGGCCCAGAGGGGCAAAATGTCCAGGTCCCCCGCTGGGAAGGACACACTATACCCTAT |
| | | GGCAAGCCAGGGTGGGCGACTTCCCATGGATCGGGTGGAGGGGGGTATCTTTCAGGATCGGCGGGCGGTCTAGGGGAACAATTCGTGG |
| | | TGGCGATGATTTGCATAGCGCGGGTCTTGGGATGCGCGCGGTTCCGAGCCAGCCTCGCACAGCTCGCTTCCGGAGCTGCGAGCTCAGG |
| | | TTTCCACCCCCGATCCCCCGGGCTTTCCTCGCACCGCTGAGCCCAGCTTGTGGGGTGCACTCGACCAACGCCCGACAGGGCTGGGGAA |
| | | TGTGACAGGCAGCAGGTTCACCCGGGCTTGGGGAGGGGGAGTTTCCGCTTTGACAGCATTTTCCTTTGCCGTCTGCTGGTGGATTCCTAT |
| | | TCCCAGTCGGTAATCGCCCCGCAGTGTTGATCTAAGAAGGTAAAGAAAACTAGGTTTCCCTGCAAAGAGCCTCCCCCAAATCGGCGGACT |
| | | CCGGATACTTTGAGTGGATTTAGAAATTTATGTAATCTTTCTCCTTTAGTTTATTTCATCCTCTCCTACAGTTTTCTCTGATTTGCTGTT |
| | | GGTTCGGGGCAAGATAAAGCAGCCAGTAGAGAGCGATAATAATAGCGGCGGGAAATGAACTGGAGACTGGCTGACAGTTCTTAACATTTT |
| | | GTCATAGATCCCCCCGAATGTCCCAGGCTGTCTCTGGTGGGTTTTAGTACCCGCCGGCTTCTTGGGCACCGGGGACCAGAAGGAACTTGG |
| | | CAGCTGGTCTTAGGGGTACAGTTAAAGGCAGGATGACAGCTATTCTCCTGCTCATCTCAGAGCGCTGCCGCCCCCTCATGCCGGTCGCGC |
| | | AAAGAACACAGCTTTTAAAAAACACGTGCCTTCTGCCCATATAGGTCTGAAAGTGATGAGGAAAGTAATGCTTCGCCTATTAGCGAGTTTC |
| | | AGCTTTTAAAATGATCCCAAGCGTTGCTGAGATGAGAAAGCGTGGCATCCCGGGGGTCCTCAGCCCCACCCGCGCCCATGGTGCAAGTCT |
| | | GCAGGGACAGGCCCGGGACAGCACTGCCCACGCTGCTAGATTTTCCGCAGAGGATCGCTGAAGCTGCCTTCGTGGGAGACAGAATGCC |
| | | TCCTCCAGCGAGTGGAAAAGGCCTGCTGAGGACCCCGCTTTGCTCGAGCATTCAAATGTGTGTCTGTTTTATTACCCTGGGTTGAAAAGG |
| | | GACAAGAGCTTTAGCCTTTTTATCTGGCCATTTTATCAGCAACTACAAGTGTGTTGAGTGGTTATTATTACATAGGAGGCTTTTCAGTTT |
| | | GGGGTCAGTAGATCAGTCTCTTCAGACACTGATGCAGAAGCTGGGACTGGTAAGTAGGTATTATGTGCTCGGAGCGCTAGGGGACAGGAG |
| | | CAAATGGAGAAGAAAAGCGGAGGCTTTCTCCGCCCGGAGTATCGATCGGAATCCCCGCCGGTACGCCGCAGAGGGCCCTCGCCGTTGGG |
| | | CCCCGGGGGTTTAACAAGCCCAGCCGCTCCGCAGGCGGCTCGGCCGGACTCTCAGACCGGTGCCTGGAAGACACCGTCCCTGCCCCCC |
| | | TCCCGCCAAACCTGCCTCTTCTCTTTCTCTCATAGGTTATAGGTTCCCTTTCTCTCTCATTTTGGCCCCGCCCCCGGGTCCTGCCAAACAG |
| | | CCAAGCAGGCCGGGGTTTAGGGGGCTCAGAATGAAGAGGTCTGATTTGGCCAGCGCCGGCAAAGCTCACCCTTAGGCGAGGTCACAAC |
| | | AGAGGCAGGTCCTTCCTGCCCAGCCTGCCGGTGTAGTCACAGCCAAGGGTGGCACTTGAAAGGAAAAGGGAGAAAACTTCGGAGAAATT |
| | | TAGATTGCCCCAACGTTAGATTTCAGAGAAATTGACTCCAAATGCACGGATTCGTTCGGAAAGGGCGGCTAAGTGGCAGGTGGTTGCAAC |
| | | CCCGCCCGGTCGGGCCTTCGCAGAGGTTCCCCAAGACCAGCCCTTGCAGGGCGGTTTTCAGCAACCTGACAAGAGGCGGCCAAGACAA |
| | | ATTTCTGCGGGTTCGAGCACACACTCTCGGGCGTTGGGCCCCAGAGACCTCTAAACCAAGCACAAACAAGAAGGGAGTGAGAGAACCCA |
| | | GGCTAGAACTTGCACGGGCATCCCACTGAGGAAAAGCGAGGCCTCGGTGGCAGGCATGTTTTCTTCCGACGCCCGAAAATCGAGCCGAG |
| | | CGCCCGACTACATTTACTGCAGAGGTTTCCGCCTCCAGTGAGCCCGGATCCCCCAGCGGCCTGCCCGGAGCTGGTCTCCAGTCCCCGCC |
| | | GTAGTCCGACGCACGGCCCTCTCCTGGCAGCAAGCTCCCAGCGGCCAGTCTGAAGCCAATTCTGTTCAGGCGGCCGAGGGCCCTTAGC |
| | | CAACCCACCATGATGTCGCCTGGGCCACCTGATGCCCGCAGCGGCGGGACACGGCCCGGGCAGTGCGCAGTGGCTCCTGCTAGGGGC |
| | | ACCGCGTGCGTGCTTGTCTCCCGCTGCGCCGGGGACGTCCTTGGGTGACACGGGCCGCTGGGCACCTCCCAAGCCGAGGAAACGGAC |
| | | CCCCTTCGCAGAGTCTCGCGCCCACCCCCCAACCTCCCACCTCGTTTCTCGCTGCTAGGGCTCCCGACTCAGCCCACCTCTCCTGGCGG |
| | | TTTAGTTAGGGATCAGAGCTGGAGAGGCTGAACGCAACCCGTGCCAGTACGGAACAGACGATATGTTTGCCTGCTAGCTGCTTGGATGAA |
| | | TAATTGAAAAGTTCGCTGCAGTCTGTGCTTCGTCAAGTCCCGGGTGCCGGGAGAACACCTTCCCAACACGCATCAGGGTGGGCGGGAGC |
| | | GGGCAGAGGAGGCGGGACCCGAGGGAGGAGAGTGAACCCGAGCAGGAGAAGCAGCCCAGGCAGCCAGGCGCCCTCGATGCGAGAGGCT |
| | | GGGCATTTATTTTTATTCCAGGCTTTCCACTGTGTGGTTATGTCACTTTCTCAAACAAATGTGTATATGGAGGGAGATCGATGCTGATAA |
| | | TGTTTAGAAGATTAAAAGAGCATTAATGCTGGCAACAATAACGTAAACGTGTGGACCCAGATTTCATTGATCTGGAACTTGATCCGGCGC |
| | | GTTTCCAGTAAGCCCGACGGCGCGCTCTTCCCAGCAGAGCGCTCACCAGCGCCACGGCCCCGCGGTTTTCCAGCGGTGCCGCTTCGCCA |
| | | GCTCTGCGCGGGTTCTCCCGTCTGACCGCAGCTCCTCCCCCGCGAGGCCCCAGCCCGCCTTACTTCCCCGAGGTTTTCTCCTCCTCTCG |
| | | CGGGGCTCTCTGCCCTCTGCACCCCCTCCCCCGACCTCTGCACCACCCGCCCCTGTGCGCACACACCGCTACTTGCGCTTCCGGCGATC |
| | | CGCCTG |
| 27 | RAX | AACCGGAGATCTGCTTGGTGAACTGAGAGGAGTCCTTAGGAGAGCGGGGACGCCAGGGGCCGGGGGACACTTCGCTCTCGCCCTAGGG |
| | | AAGGTGGTCTTGACGCTTTCTATTGAAGTCAAACTTGAAAATATCAGCTGCCGCTGGACTAT |
| 28 | chr18 | CGTGAGCAGAACGCCCGCCCTGGAGCAGTTAGGACCGAAGGTCTCCGGAGAGTCGCCGGCGGTGCCAGGTAACGCAGAGGGCTCGGG |
| | group- | TCGGGCCCCGCTTCTGGGGCTTGGGACTCCGGGCGCGCGGAGCCAGCCCTCTGGGGCGAAATCCCCGGGCGGCGTGCGCGGTCCCTC |
| | 00277 | TCCGCGCTGTGCTCTCCCAGCAACTCCCTGCCACCTCGACGAGCCTACCGGCCGCTCCGAGTTCGACTTCCTCGGACTTAGTGGGAGAA |
| | | GGGGTTGGAAATGGGCTGCCGGGACTGGGGGAGCTGCTCTCTGGAAGCAGGGAAGCTGGGGCGCACCGGGGCAGGT |
| 29 | NETO1 | TAGAAGAGGAAGACTCCTCTGGCCCCACTAGGTATCATCCGCGCTCTCCCGCTTTCCACCTGCGCCCTCGCTTGGGCCAATCTCTGCCGC |
| | | ACGTGTCCATCCCTGAACTGCACGCTATCCTCCACCCCCGGGGGGTTCCTGCGCACTGAAAGACCGTTCTCCGGCAGGTTTTGGGATCC |
| | | GGCGACGGCTGACCGCGCGCCGCCCCCACGCCCGGTTCCACGATGCTGCAATACAGAAAGTTTACGTCGGCCCCGACCCGCGCGGGAC |
| | | TGCAGGGTCCGCCGGAGCGCGGCGCAGAGGCTTTTCCTGCGCGTTCGGCCCCGGGAAAGGGGCGGGAGGGCTGGCTCCGGGAGCGC |
| | | ACGGGCGCGGCGGGGAGGGTACTCACTGTGAAGCACGCTGCGCCCATGGATCATGTCTGTGCGTTACACCAGAGGCTCCGGGCTCCAC |
| | | TAATTCCATTTAGAGACGGGAAGACTTCCAGTGGCGGGGGGAGGACAGGGTCGAGAGGTGTTAAAGACGCAAAGCAAGAAGGAAATAAA |
| | | GGGGGGCCGAGAGGGAGACCGAGAGGAAGGGGGAGCTCCGAGCCCACGCTGCAGCCAGATCCGGATGAGTCCGTCCTCCGCCCCGG |
| | | GCGGGCTCTCGCTCTCGCTGGCCCTCAGCGCCGCGCAGCCAGCAGCATCCCCACCGTGACGCTCGCATCACACCCGGGCGCCGGCCG |
| | | CCACCATCCGCGCCGCCGCCGTCAGGACCCTCCTCCCGGGCATCGTCGCCGCCGCGGGGTCGGGAGGACGCGGCGCGCGGGAGGCG |
| | | GCGGTCGCAGGGCGAGCCCCGGGACGCCCCGAGCCGGGGCCGGGGCCGGGGAGAGGGCGCAGCGAGGTGGGGGCCAGTCCAGACC |
| | | GACGGCAGCGACGGAGCGGGCGGCGGCGGCGGCGCCGGCGGCGGCGGGGTGGCTCAGTCCCCAGTCTCAGACGCGCCGCGCAGCA |
| | | GGTCGGAGCAGCCTCCCCGGGAGGATGTCCAGCGGCAGCGCTCCTCGCTCCAGCCCTTGGGGATCTTCCGCTGAGGCATTGAAGGCAG |
| | | GAAGAAGGGGTCCGTCATCGGCTCGCCGGGCTGCGCGCCACCTCTGCTATCTTGCGGAAAGAGGAGCGGGTGGGTGGGCGTCTGGGA |
| | | GGCGGGCTGGAGGGCGGTGCAGGGGAGCGGGGCGGCCGGGGGGGGGGCCGGGGGGCGGGGAAGGGAGGGAGGAGAAAGGAGCCG |
| | | GAAGAGGGCAGAGTTACCAAATGGGCTCCTTAGTCATGGCTTGGGGCTCCACGACCCTCCTGGAAGCCCGGAGCCTGGGTGGGATAGC |
| | | GAGGCTGCGCGCGGCCGGCGCCCCGGGGCTGGTGCGCGGCAGAATGGGGCCGCGGCGGCGGCAGCAAGGACATCCCAGCCGCGCG |
| | | GATCTGGGGGAGGGGCGGGGAGGGGGTGAGGACCCGGCTGGGATCCGCGGCTCGGCCCGCCAGGGCGCAGAGAGAGGATGCAGCCG |
| | | CAAATCCCGAGCCGGATCCTCGTGCCGGACGGAAGGCGTGGAAGCGGGAGGGGCCTTCGTGTGAAAATCCCTTGTGGGGTTTGGTGTTT |
| | | CACTTTTTAAAGGTTAGACCTTGCGGGCTCTCTGCCTCCCACCCCTTCTTTTCCATCCGCGTAAAGGAACTGGGCGCCCCCTCTCCCTC |
| | | CCTCCCTGGGGCGCAGGTTTCGCCGCGGACTCCGCGCTCAGCTTGGGAGACACGGCAGGGGCGCGCCCCAGGGAAAGGCGGCCGTAAAA |
| | | GTTTCGCGGTTGAGCACTGGGCCTGATGTCCAGTCCCCCCACCAAATTACTCCTGCAAAGACGCGGGCTTCTTGCAATTGAGCCCCCCAC |
| | | CTCGAGGTATTTAAAACCACCCCAAGGCACACACGGACCCCCGTTCCCCCGCGCCACTTCCTCCTACAGGCTCGCGCGGCGCGTTAAAG |
| | | TCTGGGAGACACGAGTTGCGGGGAAACAGCACCGGAAG |
| 30 | MBP | AAGAAACAGCTCATTTCGGAGCTGAGGACAAGGCGTGGGAAGAAGACGCGTTTGGTTTCACCCAGGCGGGTGGCGGCAAAGCTGTGGG |
| | | ATGCGCGCTGCACACTCCTTCCGTCATCCCGTTCCCACCTTCCACACACACCTGCGGGAGGTCGGACATGTCCTGATTGCGTGTTCATCA |
| | | CGATGGCAAACCGAACATGAGGAGAACGCCACTGACGCTGGGTGCGCCGGCTTTCCCAGCCCTCGTGCATAACGGGGAGGGAGATGCA |
| | | GAAGTTTTTTCCAACATCGGTGCAAAGGGGAAGCTGAGGTTTTCCTAT |
| 31 | NFATC1 | TCTGTCAGCTGCTGCCATGGGGCAGCGGGAAGGCCCTGGAGGGTGCCTGGGCTGTGTCTGGTCCCGGCCACGCGTCCCTGCAGCGTCT |
| | | GAGACCTTGTGGAACACACTTGACCCGGCGCTGGGACGGGGTCGGCCCACACGCACCGCCAGCCCGCAGGAGTGAGGTGCAGGCTGC |
| | | CGCTGGCTCCTTAGGCCTCGACAGCTCTCTTGAGGTCGGCCCTCCTCCCCTCCCGAGAGCTCAGCAGCCGCAGACCCAGGCAGAGAGA |
| | | GCAAAGGAGGCTGTGGTGGCCCCCGACGGGAACCTGGGTGGCCGGGGGACACACCGAGGAACTTTCCGCCCCCCGACGGGCTCTCCC |
| | | ACCGAGGCTCAGGTGCTCGTGGGCAGCAAGGGGAAGCCCCATGGCCATGCCGCTTCCCTTTCACCCTCAGCGACGCGCCCTCCTGTGC |
| | | CCGCGGGGAACAAGACGGCTCTCGGCGGCCATGCAGGCGGCCTGTCCCACGAACACGATGGAGACCTCAGACGCCGTCCCCACCCTGT |
| | | CACTGTCACCATCACCCATCCTGTCCCCTCACGCCTCCCCACATCCCATCATTACTAC |
| 32 | chr18 | GAAGTAGAATCACAGTAAATGAGGAGTTAGGGAATTTAGGGTAGAGATTAAAGTAATGAACAGAGGAGGAGGCCTGAGACAGCTGCAGAG |
| | group- | AGACCCTGTGTTCCCTGTGAGGTGAAGCGTCTGCTGTCAAAGCCGGTTGGCGCTGAGAAGAGGTACCGGGGGCAGCACCCGCCTCCTG |
| | 00430 | GGAGAGGGATGGGCCTGCGGGCACCTGGGGGAACCGCACGGACACAGACGACACTATAAACGCGGGCGAGACATCAGGGACCGGGAA |
| | | ACAGAAGGACGCGCGTTTCGAGCAGCTGCCCAGTGGGCCACAAGCCCCGCCACGCCACAGCCTCTTCCCCTCAGCACGCAGAGA |
| 33 | OLIG2 | TACTCCGGCGACGGGAGGATGTTGAGGGAAGCCTGCCAGGTGAAGAAGGGGCCAGCAGCAGCACAGAGCTTCCGACTTTGCCTTCCAG |
| | | GCTCTAGACTCGCGCCATGCCAAGACGGGCCCCTCGACTTTCACCCCTGACTCCCAACTCCAGCCACTGGACCGAGCGCGCAAAGAACC |
| | | TGAGACCGCTTGCTCTCACCGCCGCAAGTCGGTCGCAGGACAGACACCAGTGGGCAGCAACAAAAAAAGAAACCGGGTTCCGGGACAC |
| | | GTGCCGGCGGCTGGACTAACCTCAGCGGCTGCAACCAAGGAGCGCGCACGTTGCGCCTGCTGGTGTTTATTAGCTACACTGGCAGGCG |
| | | CACAACTCCGCGCCCCGACTGGTGGCCCCACAGCGCGCACCACACATGGCCTCGCTGCTGTTGGCGGGGTAGGCCCGAAGGAGGCATCT |
| | | ACAAATGCCCGAGCCCTTTCTGATCCCCACCCCCCCGCTCCCTGCGTCGTCCGAGTGACAGATTCTACTAATTGAACGGTTATGGGTCA |
| | | TCCTTGTAACCGTTGGACGACATAACACCACGCTTCAGTTCTTCATGTTTTAAATACATATTTAACGGATGGCTGCAGAGCCAGCTGGGA |
| | | AACACGCGGATTGAAAAATAATGCTCCAGAAGGCACGAGACTGGGGCGAAGGCGAGAGCGGGCTGGGCTTCTAGCGGAGACCGCAGAGGG |
| | | AGACATATCTCAGAACTAGGGGCAATAACGTGGGTTTCTCTTTGTATTTGTTTATTTTGTAACTTTGCTACTTGAAGACCAATTATTTAC |
| | | TATGCTAATTTGTTTGCTTGTTTTTAAAACCGTACTTGCACAGTAAAAGTTCCCCAACAACGGAAGTAACCCGACGTTCCTCACACTCCC |
| | | TAGGAGACTGTGTGCGTGTGTGCCCGCGCGTGCGCTCACAGTGTCAAGTGCTAGCATCCGAGATCTGCAGAAACAAATGTCTGAATTCGA |
| | | AATGTATGGGTGTGAGAAATTCAGCTCGGGGAAGAGATTAGGGACTGGGGGAGACAGGTGGCTGCCTGTACTATAAGGAACCGCCAACGC |
| | | CAGCATCTGTAGTCCAAGCAGGGCTGCTCTGTAAAGGCTTAGCAATTTTTTCTGTAGGCTTGCTGCACACGGTCTCTGGCTTTTCCCATC |
| | | TGTAAAATGGGTGAATGCATCCGTACCTCAGCTACCTCCGTGAGGTGCTTCTCCAGTTCGGGCTTAATTCCTCATCGTCAAGAGTTTTCA |
| | | GGTTTCAGAGCCAGCCTGCAATCGGTAAAACATGTCCCAACGCGGTCGCGAGTGGTTCCATCTCGCTGTCTGGCCCACAGCGTGGAGAAG |
| | | CCTTGCCCAGGCCTGAAACTTCTCTTTGCAGTTCCAGAAAGCAGGCGACTGGGACGGAAGGCTCTTTGCTAACCTTTTACAGCGGAGCCC |
| | | TGCTTGGACTACAGATGCCAGCGTTGCCCCTGCCCCAAGGCGTGTGGTGATCACAAAGACGACACTGAAAATACTTACTATCATCCGGCT |
| | | CCCCTGCTAATAAATGGAGGGGTGTTTAACTACAGGCACGACCCTGCCCTTGTGCTAGCGCGGTTACCGTGCGGAAATAACTCGTCCCTG |
| | | TACCCACACCATCCTCAACCTAAAGGAGAGTTGTGAATTCTTTCAAAACACTCTTCTGGAGTCCGTCCCCTCCCTCCTTGCCCGCCCTCT |
| | | ACCCCTCAAGTCCCTGCCCCCAGCTGGGGGCGCTACCGGCTGCCGTCGGAGCTGCAGCCACGGCCATCTCCTAGACGCGCGAGTAGAGCA |
| | | CCAAGATAGTGGGGACTTTGTGCCTGGGCATCGTTTACATTTGGGGCGCCAAATGCCCACGTGTTGATGAAACCAGTGAGATGGGAACAG |
| | | GCGGCGGGAAACCAGACAGAGGAAGAGCTAGGGAGGAGACCCCAGCCCCGGATCCTGGGTCGCCAGGGTTTTCCGCGCGCATCCCAAAAG |
| | | GTGCGGCTGCGTGGGGCATCAGGTTAGTTTGTTAGACTCTGCAGAGTCTCCAAACCATCCCATCCCCCAACCTGACTCTGTGGTGGCCGT |
| | | ATTTTTTACAGAAATTTGACCACGTTCCCTTTCTCCCTTGGTCCCAAGCGCGCTCAGCCCTCCCTCCATCCCCCTTGAGCCGCCCTTCTC |
| | | CTCCCCCTCGCCTCCTCGGGTCCCTCCTCCAGTCCCTCCCCAAGAATCTCCCGGCCACGGGCGCCCATTGGTTGTGCGCAGGGAGGAGG |
| | | CGTGTGCCCGGCCTGGCGAGTTTCATTGAGCGGAATTAGCCCGGATGACATCAGCTTCCCAGCCCCCCGGCGGGCCCAGCTCATTGGC |
| | | GAGGCAGCCCCTCCAGGACACGCACATTGTTCCCCGCCCCCGCCCCCGCCACCGCTGCCGCCGTCGCCGCTGCCACCGGGCTATAAAA |
| | | ACCGGCCGAGCCCCTAAAGGTGCGGATGCTTATTATAGATCGACGCGACACCAGCGCCCGGTGCCAGGTTCTCCCCTGAGGCTTTTCGG |
| | | AGCGAGCTCCTCAAATCGCATCCAGAGTAAGTGTCCCCGCCCCACAGCAGCCGCAGCCTAGATCCCAGGGACAGACTCTCCTCAACTCG |
| | | GCTGTGACCCAGAATGCTCCGATACAGGGGGTCTGGATCCCTACTCTGCGGGCCATTTCTCCAGAGCGACTTTGCTCTTCTGTCCTCCCC |
| | | ACACTCACCGCTGCATCTCCCTCACCAAAAGCGAGAAGTCGGAGCGACAACAGCTCTTTCTGCCCAAGCCCCAGTCAGCTGGTGAGCTC |
| | | CCCGTGGTCTCCAGATGCAGCACATGGACTCTGGGCCCCGCGCCGGCTCTGGGTGCATGTGCGTGTGCGTGTGTTTGCTGCGTGGTGTC |
| | | GATGGAGATAAGGTGGATCCGTTTGAGGAACCAAATCATTAGTTCTCTATCTAGATCTCCATTCTCCCCAAAGAAAGGCCCTCACTTCCC |
| | | ACTCGTTTATTCCAGCCCGGGGGCTCAGTTTTCCCACACCTAACTGAAAGCCCGAAGCCTCTAGAATGCCACCCGCACCCCGAGGGTCAC |
| | | CAACGCTCCCTGAAATAACCTGTTGCATGAGAGCAGAGGGGAGATAGAGAGAGCTTAATTATAGGTACCCGCGTGCAGCTAAAAGGAGG |
| | | GCCAGAGATAGTAGCGAGGGGGACGAGGAGCCACGGGCCACCTGTGCCGGGACCCCGCGCTGTGGTACTGCGGTGCAGGCGGGAGCAGC |
| | | TTTTCTGTCTCTCACTGACTCACTCTCTCTCTCTCTCCCTCTCTCTCTCTCTCATTCTCTCTCTTTTCTCCTCCTCTCCTGGAAGTTTT |
| | | CGGGTCCGAGGGAAGGAGGACCCTGCGAAAGCTGCGACGACTATCTTCCCCTGGGGCCATGGACTCGGACGCCAGCCTGGTGTCCAGCC |
| | | GCCCGTCGTCGCCAGAGCCCGATGACCTTTTTCTGCCGGCCCGGAGTAAGGGCAGCAGCGGCAGCGCCTTCACTGGGGGCACCGTGTCC |
| | | TCGTCCACCCCGAGTGACTGCCC |
| 34 | SIM2 | TTAATTCGAAAATGGCAGACAGAGCTGAGCGCTGCCGTTCTTTTCAGGATTGAAAATGTGCCAGTGGGCCAGGGGCGCTGGGACCCGCG |
| | | GTGCGGAAGACTCGGAACAGGAAGAAATAGTGGCGCGCTGGGTGGGCTGCCCCGCCGCCCACGCCGGTTGCCGCTGGTGACAGTGGC |
| | | TGCCCGGCCAGGCACCTCCGAGCAGCAGGTCTGAGCGTTTTTGGCGTCCCAAGCGTTCCGGGCCGCGTCTTCCAGAGCCTCTGCTCCCA |
| | | GCGGGGTCGCTGCGGCCTGGCCCGAAGGATTTGACTCTTTGCTGGGAGGCGCGCTGCTCAGGGTTCTG |
| 35 | SIM2 | CCGGTCCCCAGTTTGGAAAAAGGCGCAAGAAGCGGGCTTTTCAGGGACCCCGGGGAGAACACGAGGGCTCCGACGCGGGAGAAGGATT |
| | | GAAGCGTGCAGAGGCGCCCCAAATTGCGACAATTTACTGGGATCCTTTTGTGGGGAAAGGAGGCTTAGAGGCTCAAGCTATAGGCTGTC |
| | | CTAGAGCAACTAGGCGAGAACCTGGCCCCAAACTCCCTCCTTACGCCCTGGCACAGGTTCCCGGCGACTGGTGTTCCCAAGGGAGCCCC |
| | | CTGAGCCTACCGCCCTTGCAGGGGGTCGTGCTGCGGCTTCTGGGTCATAAACGCCGAGGTCGGGGGTGGCGGAGCTGTAGAGGCTGCC |
| | | CGCGCAGAAAGCTCCAGGATCCCAATATGTG |
| 36 | DSCR6 | GCGCAGGTCCCCCCAGTCCCCGAGGGAGTGCGCCCGACGGAAACGCCCCTAGCCCGCGGGCCTCGCTTTCCTCTCCCGGGTTCCTGG |
| | | GTCACTTCCCGCTGTCTC |
| 37 | DSCAM | TTCCCTCGCGGCTTTGGAAAGGGGGTGCAAATGCACCCTTCTGCGGGCCCGCTACCCGCTGCAACACCTGTGTTTCCTTTCTGGGCACCT |
| | | TCTAGGTTTCTAGATATTGCTGTGAATACGGTCCTCCGCTGTACAGTTGAAAACAAA |
| 38 | chr21 | TGGGAATTTAGGTCGGGCACTGCCGATATGTCGCCTTCCACAAGGCGGGCCCGGGCCTCTGCTGACCGTGCACCGGTCCTGGGGCTGG |
| | group- | GTAATTCTGCAGCAGCAGCGCAGCCCATGCCGGGGAATTTGCGGGCAGAGGAGACAGTGAGGCCCGCGTTCTGTGCGGGAACTCCCGA |
| | 00165 | GCTCACAGAGCCCAAGACCACACGGCTGCATCTGCTTGGCTGACTGGGCCAGGCCCACGCGTAGTAACCCGGACGTCTCTCTCTCACAG |
| | | TCCCCTTGCGTCTGGCCAGGGAGCTGCCAGGCTGCACCCCGCGGTGGGGATCGGGAGAGGGGCAGTGTCGCCCATCCCCGGAAGGCT |
| | | GAGCCTGGTGCAG |
| 39 | PRMT2 | CGGTTTTCTCCTGGAGGACTGTGTTCAGACAGATACTGGTTTCCTTATCCGCAGGTGTGCGCGGCGCTCGCAAGTGGTCAGCATAACGCC |
| | | GGGCGAATTCGGAAAGCCCGTGCGTCCGTGGACGACCCACTTGGAAGGAGTTGGGAGAAGTCCTTGTTCCCACGCGCGGACGCTTCCC |
| | | TCCGTGTGTCCTTCGAGCCACAAAAAGCCCAGACCCTAACCCGCTCCTTTCTCCCGCCGCGTCCATGCAGAACTCCGCCGTTCCTGGGA |
| | | GGGGAAGCCCGCGAGGCGTCGGGAGAGGCACGTCCTCCGTGAGCAAAGAGCTCCTCCGAGCGCGCGGCGGGGACGCTGGGCCGACA |
| | | GGGGACCGCGGGGGCAGGGCGGAGAGGACCCGCCCTCGAGTCGGCCCAGCCCTAACACTCAGGAC |
| 40 | SIX2 | AGGGAATCGGGCTGACCAGTCCTAAGGTCCCACGCTCCCCTGACCTCAGGGCCCAGAGCCTCGCATTACCCCGAGCAGTGCGTTGGTTA |
| | | CTCTCCCTGGAAAGCCGCCCCCGCCGGGGCAAGTGGGAGTTGCTGCACTGCGGTCTTTGGAGGCCTAGGTCGCCCAGAGTAGGCGGAG |
| | | CCCTGTATCCCTCCTGGAGCCGGCCTGCGGTGAGGTCGGTACCCAGTACTTAGGGAGGGAGGACGCGCTTGGTGCTCAGGGTAGGCTG |
| | | GGCCGCTGCTAGCTCTTGATTTAGTCTCATGTCCGCCTTTGTGCCGGCCTCTCCGATTTGTGGGTCCTTCCAAGAAAGAGTCCTCTAGGG |
| | | CAGCTAGGGTCGTCTCTTGGGTCTGGCGAGGCGGCAGGCCTTCTTCGGACCTATCCCCAGAGGTGTAACGGAGACTTTCTCCACTGCAG |
| | | GGCGGCCTGGGGCGGGCATCTGCCAGGCGAGGGAGCTGCCCTGCCGCCGAGATTGTGGGGAAACGGCGTGGAAGACACCCCATCGGA |
| | | GGGCACCCAATCTGCCTCTGCACTCGATTCCATCCTGCAACCCAGGAGAAACCATTTCCGAGTTCCAGCCGCAGAGGCACCCGCGGAGT |
| | | TGCCAAAAGAGACTCCCGCGAGGTCGCTCGGAACCTTGACCCTGACACCTGGACGCGAGGTCTTTCAGGACCAGTCTCGGCTCGGTAGC |
| | | CTGGTCCCCGACCACCGCGACCAGGAGTTCCTTCTTCCCTTCCTGCTCACCAGCCGGCCGCCGGCAGCGGCTCCAGGAAGGAGCACCA |
| | | ACCCGCGCTGGGGGCGGAGGTTCAGGCGGCAGGAATGGAGAGGCTGATCCTCCTCTAGCCCCGGCGCATTCACTTAGGTGCGGGAGCC |
| | | CTGAGGTTCAGCCTGACTTTC |
| 41 | SIX2 | CACTACGGATCTGCCTGGACTGGTTCAGATGCGTCGTTTAAAGGGGGGGGCTGGCACTCCAGAGAGGAGGGGGCGCTGCAGGTTAATT |
| | | GATAGCCACGGAAGCACCTAGGCGCCCCATGCGCGGAGCCGGAGCCGCCAGCTCAGTCTGACCCCTGTCTTTTCTCTCCTCTTCCCTCT |
| | | CCCACCCCTCACTCCGGGAAAGCGAGGGCCGAGGTAGGGGCAGATAGATCACCAGACAGGCGGAGAAGGACAGGAGTACAGATGGAG |
| | | GGACCAGGACACAGAATGCAAAAGACTGGCAGGTGAGAAGAAGGGAGAAACAGAGGGAGAGAGAAAGGGAGAAACAGAGCAGAGGCGG |
| | | CCGCCGGCCCGGCCGCCCTGAGTCCGATTTCCCTCCTTCCCTGACCCTTCAGTTTCACTGCAAATCCACAGAAGCAGGTTTGCGAGCTCG |
| | | AATACCTTTGCTCCACTGCCACACGCAGCACCGGGACTGGGCGTCTGGAGCTTAAGTCTGGGGGTCTGAGCCTGGGACCGGCAAATCCG |
| | | CGCAGCGCATCGCGCCCAGTCTCGGAGACTGCAACCACCGCCAAGGAGTACGCGCGGCAGGAAACTTCTGCGGCCCAATTTCTTCCCCA |
| | | GCTTTGGCATCTCCGAAGGCACGTACCCGCCCTCGGCACAAGCTCTCTCGTCTTCCACTTCGACCTCGAGGTGGAGAAAGAGGCTGGCA |
| | | AGGGCTGTGCGCGTCGCTGGTGTGGGGAGGGCAGCAGGCTGCCCCTCCCCGCTTCTGCAGCGAGTTTTCCCAGCCAGGAAAAGGGAGG |
| | | GAGCTGTTTCAGGAATTTCAGTGCCTTCACCTAGCGACTGACACAAGTCGTGTGTATAGGAAG |
| 42 | SOX14 | GGAGCCTGAAGTCAGAAAAGATGGGGCCTCGTTACTCACTTTCTAGCCCAGCCCCTGGCCCTGGGTCCCGCAGAGCCGTCATCGCAGGC |
| | | TCCTGCCCAGCCTCTGGGGTCGGGTGAGCAAGGTGTTCTCTTCGGAAGCGGGAAGGGCTGCGGGTCGGGGACGTCCCTTGGCTGCCAC |
| | | CCCTGATTCTGCATCCTTTTCGCTCGAATCCCTGCGCTAGGCATCCTCCCCGATCCCCCAAAAGCCCAAGCACTGGGTCTGGGTTGAGGA |
| | | AGGGAACGGGTGCCCAGGCCGGACAGAGGCTGAAAGGAGGCCTCAAGGTTCCTCTTTGCTACAAAGTGGAGAAGTTGCTCTACTCTGGA |
| | | GGGCAGTGGCCTTTTCCAAACTTTTCCACTTAGGTCCGTAAGAAAAGCAATTCATACACGATCAGCGCTTTCGGTGCGAGGATGGAAAGA |
| | | AACTTC |
| 43 | TLX3 | TTTTCCTGTTACAGAGCTGAGCCCACTCATGTGGTGCCAAGTAGCGACTATCTCTCGGCCACCTCCACCCAGAGCAATGTGGGCGCCCCC |
| | | AGCGGGTGGGAGCGATTGCCGAGCGGCGCAAGGGCGTTTAACGCCTAACCCCCTCCTCCTGGGTTGCCAAGCCGCTAGGTCGCCGTTT |
| | | CCAACGTGGCTGCGCGGGACTGAAGTCCGACGACTCCTCGTCCTCAGTAGGAGACACACCTCCCACTGCCCCCAGCCACGCGAGCTATG |
| | | GGCAGAATCGGGGCAACGGTAATATCTGGATGGGGCAGGCTCCCCTGAGGCTGTGCTTAAGAAAAAAGGAATCTGGAGTAGCCTGAGGG |
| | | GCCCCACGAGGGGGCCTCCTTTGCGATCGTCTCCCAGCCTTAGGCCAAGGCTACGGAGGCAGGCGGCCGAGTGTTGGCGCCCAGCCC |
| | | GGCCGAGGACTGGATGGAGGACGAGAAGCAGCCTGCCTCTGGGCGACAGCTGCGGACGCAGCCTCGCCGCCTCGCCGCCTCAGCCTC |
| | | GGTCCCAGCGTCTCTAAAGCCGCGCCCATTTTACAGATGCAGGGCAGGGAGACAAGAGGCATCTCCGGGGGCCGAGTAGAATGATGGC |
| | | GCGGGTTCTCCCGGCGCCCTGATTTCGAGGCTGCGCCCGGGGCCCTACATGCAGGCGGGGAGGCCTGGGCCGAAGGCGTCTGCAAGG |
| | | AGGGGCGAGTCTGCCCGGTCCGGGCAGGGAGTGAGGCCACAGTCAGTTCTCCCTAGGAGGCCGCGCAGCGGGTAGGGTATGGGACTG |
| | | GGGGACGCAACGGGGACCTGGCCGAATCAGAGCCCTCAGCAGAGAACGCCGAAAACTCTGGGGCCGGCCGCTCGCTTCCCGCTAGTG |
| | | GGAATGGTTTCCGGTCATCCGTTCCCAGTCCAGCCCCGGGTAGGGAGCTCTGATTTGCAATGCACAGCACTTGCGAGGTTCGAATGCCC |
| | | CCGCAATTTGCAGATGGAAATACTAAGCCTAGGCCGGGCGTGGTGGCTCAAGCCTATCATCTCAGCCCTTTGGGAGGCCAAGCCGGGAG |
| | | GATTGTTTGAGCCCAAGAATTCAAAACCAGCCTGAGCAACATAGCGACCCCGTCTCTACAAAATAAAATAAAATAAATTATCCGGGCGTGG |
| | | TGGCACGCGCCTGTGGTTCCAGCTACTCCGGAGGCTGAGGTGGGAGGATCGCTTGAGTCCGGGAGGTCGAGGCTACAGTGAGCCGTGA |
| | | TCGCACCACTGCACTCCAGCCTGGGCGACAGAGTGAGACCTTGTCTCAAAAAAGGAAAAAAAGAAAAAGAAAGTAAGCTTCAAAGAAGCT |
| | | CTGATAATAGTTCTGGGTCGTGCAGCGGTGGCGGCCCCGCGCTCTCGCCCCTAAAGCAAGCGCTCTTTGTACTGGGTGGAGGAGCTTTG |
| | | AGTAGTGAGGGTGGAGATGCAGCTTCGGGGTGGCGCAGCCACCCTGACACTAGGCCCGGGGTCGCAGTGGGACAGAAGAGTCTGCCG |
| | | CTCTGACTTGGGCTCTGAGTTCCAAGGGCGCCCGGCACTTCTAGCCTCCCAGGCTTGCGCGCTGGCGCCTTTGCCATCCGTGCCGAAGT |
| | | GGGGAGACCTAGCCGCGACCACCACGAGCGCAGCGGTGACACCCAGAGGTCCCACCGGGCCCCTGGGCAGGGTAACCTTAGCCTGTC |
| | | CGCTTCGGCAGCTTTGCGAAGAGTGGCGCGCAGCTAGGGCTGAGGCTCTTGCGGACCTGCGGTCGAAGCAGGCGGCTGAGCCAGTTCG |
| | | ATCGCCAAGGCCTGGGCTGCCGACAGTGGTGCGCGCTCTGTTCCGCCGCGGCCGGGCCAGGCGCTCTGGAATAGCGATGGGGGGACA |
| | | CGGCCTCCAACTTTCTGCAGAGACCATCGGGCAGCTCCGGGCCTAAGCAGCGACCTCACCGAAGGTTCCTGGGAACCTTTGCCAAAATC |
| | | CCAGCCTCTGCCTCGGTCCAGCTAAACCGTGTGTAAACAAGTGCACCAAG |
| 44 | FOXP4 | ATAAAGGACCGGGTAATTTCGCGGAATGCGGATTTTGAGACAGGCCCAGACGGCGGCGGATTCCCTGTGTCCCCCAACTGGGGCGATCT |
| | | CGTGAACACACCTGCGTCCCACCCCGATCCTAGGTTGGGGGGAAAGGGTATGGGAACCCTGAGCCCAGAGCGCGCCCCGCTCTTTCCTT |
| | | TGCTCCCCGGCTTCCCTGGCCAGCCCCCTCCCGGCTGGTTTCCTCGCTCACTCGGCGCCTGGCGTTTCGGGCGTCTGGAGATCACCGC |
| | | GTGTCTGGCACCCCAACGTCTAGTCTCCCCGCAGGTTGACCGCGGCGCCTGGAGCCGGGAATAGGGGTGGGGAGTCCGGAGAACCAAA |
| | | CCCGAGCCTGAAGTTGCCATTCGGGTGACTCCCGAGAAAGCCCGGGAGCATTTTGGCCAATGCGGGTTTTTACCTGAACTTCAGCATCTT |
| | | CACC |
| 45 | FOXP4 | AATTGGAAAACCCTGGTATTGTGCCTGTTTGGGGGAAGAAAACGTCAATAAAAATTAATTGATGAGTTGGCAGGGCGGGCGGTGCGGGTT |
| | | CGCGGCGAGGCGCAGGGTGTCATGGCAAATGTTACGGCTCAGATTAAGCGATTGTTAATTAAAAAGCGACGGTAATTAATACTCGCTACG |
| | | CCATATGGGCCCGTGAAAAGGCACAAAAGGTTTCTCCGCATGTGGGGTTCCCCTTCTCTTTTCTCCTTCCACAAAAGCACCCCAGCCCGT |
| | | GGGTCCCCCCTTTGGCCCCAAGGTAGGTGGAACTCGTCACTTCCGGCCAGGGAGGGGATGGGGCGGTCTCCGGCGAGTTCCAAGGGC |
| | | GTCCCTCGTTGCGCACTCGCCCGCCCAGGTTCTTTGAA |
| 46 | chr7 | GGGAAGCGATCGTCTCCTCTGTCAACTCGCGCCTGGGCACTTAGCCCCTCCCGTTTCAGGGCGCCGCCTCCCCGGATGGCAAACACTAT |
| | group- | AAAGTGGCGGCGAATAAGGTTCCTCCTGCTGCTCTCGGTTTAGTCCAAGATCAGCGATATCACGCGTCCCCCGGAGCATCGCGTGCAGG |
| | 00267 | AGCCATGGCGCGGGAGCTATACCACGAAGAGTTCGCCCGGGCGGGCAAGCAGGCGGGGCTGCAGGTCTGGAGGATTGAGAAGCTGGA |
| | | GCTGGTGCCCGTGCCCCAGAGCGCTCACGGCGACTTCTACGTCGGGGATGCCTACCTGGTGCTGCACACGGCCAAGACGAGCCGAGGC |
| | | TTCACCTACCACCTGCACTTCTGGCTCGGTAAGGGACGGCGGGCGGCGGGACCCCGACGCACCAAGGCCGGCGAGGGGAGGGCGTAG |
| | | GGGTCTGAGATTTGCAGGCGTGGGAGTAAAGGGGACCGCAAACTGAGCTAG |
| 47 | NPY | CTCAGGGGCGGGAAGTGGCGGGTGGGAGTCACCCAAGCGTGACTGCCCGAGGCCCCTCCTGCCGCGGCGAGGAAGCTCCATAAAAGC |
| | | CCTGTCGCGACCCGCTCTCTGCACCCCATCCGCTGGCTCTCACCCCTCGGAGACGCTCGCCCGACAGCATAGTACTTGCCGCCCAGCCA |
| | | CGCCCGCGCGCCAGCCACCGTGAGTGCTACGACCCGTCTGTCTAGGGGTGGGAGCGAACGGGGCGCCCGCGAACTTGCTAGAGACGC |
| | | AGCCTCCCGCTCTGTGGAGCCCTGGGGCCCTGGGATGATCGCGCTCCACTCCCCAGCGGACTATGCCGGCTCCGCGCCCCGACGCGGA |
| | | CCAGCCCTCTTGGCGGCTAAATTCCACTTGTTCCTCTGCTCCCCTCTGATTGTCCACGGCCCTTCTCCCGGGCCCTTCCCGCTGGGCGGT |
| | | TCTTCTGAGTTACCTTTTAGCAGATATGGAGGGAGAACCCGGGACCGCTATCCCAAGGCAGCTGGCGGTCTCCCTGCGGGTCGCCGCCT |
| | | TGAGGCCCAGGAAGCGGTGCGCGGTAGGAAGGTTTCCCCGGCAGCGCCATCGAGTGAGGAATCCCTGGAGCTCTAGAGCCCCGCGCCC |
| | | TGCCACCTCCCTGGATTCTTGGGCTCCAAATCTCTTTGGAGCAATTCTGGCCCAGGGAGCAATTCTCTTTCCCCTTCCCCACCGCAGTCGT |
| | | CACCCCGAGGTGATCTCTGCTGTCAGCGTTGATCCCCTGAAGCTAGGCAGACCAGAAGTAACAGAGAAGAAACTTTTCTTCCCAGACAAG |
| | | AGTTTGGGCAAGAAGGGAGAAAAGTGACCCAGCAGGAAGAACTTCCAATTCGGTTTTGAATGCTAAACTGGCGGGGCCCCCACCTTGCAC |
| | | TCTCGCCGCGCGCTTCTTGGTCCCTGAGACTTCGAACGAAGTTGCGCGAAGTTTTCAGGTGGAGCAGAGGGGCAGGTCCCGACCGGAC |
| | | GGCGCCCGGAGCCCGCAAGGTGGTGCTAGCCACTCCTGGGTTCTCTCTGCGGGACTGGGACGAGAGCGGATTGGGGGTCGCGTGTGG |
| | | TAGCAGGAGGAGGAGCGCGGGGGGCAGAGGAGGGAGGTGCTGCGCGTGGGTGCTCTGAATCCCCAAGCCCGTCCGTTGAGCCTTCTG |
| | | TGCCTGCAGATGCTAGGTAACAAGCGACTGGGGCTGTCCGGACTGACCCTCGCCCTGTCCCTGCTCGTGTGCCTGGGTGCGCTGGCCG |
| | | AGGCGTACCCCTCCAAGCCGGACAACCCGGGCGAGGACGCACCAG |
| 48 | SHH | TGGAGAACCTTGGGCTCTGTGGCCTCAAAGGTAGGGGTGATTTCGAGGGGCCGGCACCTCACAGGGCAGGTTCCACCGCGGAAACGCA |
| | | GTCATCGCCCAGCGACCCTGCTCCTGGCCCTCAGCCTCCCCCCAGGTTTCTTTTTCTCTTGAATCAAGCCGAGGTGCGCCAATGGCCTTC |
| | | CTTGGGTCGGATCCGGGGGGCCAGGGCCAGCTTACCTGCTTTCACCGAGCAGTGGATATGTGCCTTGGACTCGTAGTACACCCAGTCGA |
| | | AGCCGGCCTCCACCGCCAGGCGGGCCAGCATGCCGTACTTGCTGCGGTCGCGGTCAGACGTGGTGATGTCCACTGCGCGGCCCTCGTA |
| | | GTGCAGAGACTCCTCTGAGTGGTGGCCATCTTCGTCCCAGCCCTCGGTCACCCGCAGTTTCACTCCTGGCCACTGGTTCATCACCGAGAT |
| | | GGCCAAAGCGTTCAACTTGTCCTTACACCTCTGCGAAGACAAGGGGACCCCCACCGACGGACACGTTAGCCTGGGCAACCGCCACCCCT |
| | | CCCGGCCCCTCCATCAGCCT |
| 49 | OSR2 | TCTCACGACCCATCCGTTAACCCACCGTTCCCAGGAGCTCCGAGGCGCAGCGGCGACAGAGGTTCGCCCCGGCCTGCTAGCATTGGCAT |
| | | TGCGGTTGACTGAGCTTCGCCTAACAGGCTTGGGGAGGGTGGGCTGGGCTGGGCTGGGCTGGGCTGGGTGCTGCCCGGCTGTCCGCC |
| | | TTTCGTTTTCCTGGGACCGAGGAGTCTTCCGCTCCGTATCTGCCTAGAGTCTGAATCCGACTTTCTTTCCTTTGGGCACGCGCTCGCCAGT |
| | | GGAGCACTTCTTGTTCTGGCCCCGGGCTGATCTGCACGCGGACTTGAGCAGGTGCCAAGGTGCCACGCAGTCCCCTCACGGCTTTCGGG |
| | | GGGTCTTGGAGTCGGGTGGGGAGGGAGACTTAGGTGTGGTAACCTGCGCAGGTGCCAAAGGGCAGAAGGAGCAGCCTTGGATTATAGT |
| | | CACGGTCTCTCCCTCTCTTCCCTGCCATTTTTAGGGCTTTCTCTAUGTGCTGTTGTCTCACTGGGTTTTTGTCGGAGCCCCACGCCCTCCG |
| | | GCCTCTGATTCCTGGAAGAAAGGGTTGGTCCCCTCAGCACCCCCAGCATCCCGGAAAATGGGGAGCAAGGCTCTGCCAGCGCCCATCCC |
| | | GCTCCACCCGTCGCTGCAGCTCACCAATTACTCCTTCCTGCAGGCCGTGAACACCTTCCCGGCCACGGTGGACCACCTGCAGGGCCTGT |
| | | ACGGTCTCAGCGCGGTACAGACCATGCACATGAACCACTGGACGCTGGGGTATCCCAAT |
| 50 | GLIS3 | TGGTTTCCTTTCGCTTCTCGCCTCCCAAACACCTCCAGCAAGTCGGAGGGCGCGAACGCGGAGCCAGAAACCCTTCCCCAAAGTTTCTCC |
| | | CGCCAGGTACCTAATTGAATCATCCATAGGATGACAAATCAGCCAGGGCCAAGATTTCCAGACACTTGAGTGACTTCCCGGTCCCCGAGG |
| | | TGACTTGTCAGCTCCAGTGAGTAACTTGGAACTGTCGCTCGGGGCAAGGTGTGTGTCTAGGAGAGAGCCGGCGGCTCACTCACGCTTTC |
| | | CAGAGAGCGACCCGGGCCGACTTCAAAATACACACAGGGTCATTTATAGGGACTGGAGCCGCGCGCAGGACAACGTCTCCGAGACTGAG |
| | | ACATTTTCCAAACAGTGCTGACATTTTGTCGGGCCCCATAAAAAATGTAAACGCGAGGTGACGAACCCGGCGGGGAGGGTTCGTGTCTGG |
| | | CTGTGTCTGCGTCCTGGCGGCGTGGGAGGTTATAGTTCCAGACCTGGCGGCTGCGGATCGCCGGGCCGGTACCCGCGAGGAGTGTAGG |
| | | TACCCTCAGCCCGACCACCTCCCGCAATCATGGGGACACCGGCTTGGATGAGACACAGGCGTGGAAAACAGCCTTCGTGAAACTCCACA |
| | | AACACGTGGAACTTGAAAAGACAACTACAGCCCCGCGTGTGCGCGAGAGACCTCACGTCACCCCATCAGTTCCCACTTCGCCAAAGTTTC |
| | | CCTTCAGTGGGGACTCCAGAGTGGTGCGCCCCATGCCCGTGCGTCCTGTAACGTGCCCTGATTGTGTACCCCTCTGCCCGCTCTACTTG |
| | | AAATGAAAACACAAAAACTGTTCCGAATTAGCGCAACTTTAAAGCCCCGTTATCTGTCTTCTACACTGGGCGCTCTTAGGCCACTGACAGA |
| | | AACATGGTTTGAACCCTAATTGTTGCTATCAGTCTCAGTCAGCGCAGGTCTCTCAGTGACCTGTGACGCCGGGAGTTGAGGTGCGCGTAT |
| | | CCTTAAACCCGCGCGAACGCCACCGGCTCAGCGTAGAAAACTATTTGTAATCCCTAGTTTGCGTCTCTGAGCTTTAACTCCCCCACACTCT |
| | | CAAGCGCCCGGTTTCTCCTCGTCTCTCGCCTGCGAGCAAAGTTCCTATGGCATCCACTTACCAGGTAACCGGGATTTCCACAACAAAGCC |
| | | CGGCGTGCGGGTCCCTTCCCCCGGCCGGCCAGCGCGAGTGACAGCGGGCGGCCGGCGCTGGCGAGGAGTAACTTGGGGCTCCAGCC |
| | | CTTCAGAGCGCTCCGCGGGCTGTGCCTCCTTCGGAAATGAAAACCCCCATCCAAACGGGGGGACGGAGCGCGGAAACCCGGCCCAAGT |
| | | GCCGTGTGTGCGCGCGCGTCTG |
| 51 | PRMT8 | GAAAGCCATCCTTACCATTCCCCTCACCCTCCGCCCTCTGATCGCCCACCCGCCGAAAGGGTTTCTAAAAATAGCCCAGGGCTTCAAGGC |
| | | CGCGCTTCTGTGAAGTGTGGAGCGAGCGGGCACGTAGCGGTCTCTGCCAGGTGGCTGGAGCCCTGGAAGCGAGAAGGCGCTTCCTCCC |
| | | TGCATTTCCACCTCACCCCACCCCCGGCTCATTTTTCTAAGAAAAAGTTTTTGCGGTTCCCTTTGCCTCCTACCCCCGCTGCCGCGCGGG |
| | | GTCTGGGTGCAGACCCCTGCCAGGTTCCGCAGTGTGCAGCGGCGGCTGCTGCGCTCTCCCAGCCTCGGCGAGGGTTAAAGGCGTCCGG |
| | | AGCAGGCAGAGCGCCGCGCGCCAGTCTATTTTTACTTGCTTCCCCCGCCGCTCCGCGCTCCCCCTTCTCAGCAGTTGCACATGCCAGCT |
| | | CTGCTGAAGGCATCAATGAAAACAGCAGTAG |
| 52 | TBX3 | ATCGAAAATGTCGACATCTTGCTAATGGTCTGCAAACTTCCGCCAATTATGACTGACCTCCCAGACTCGGCCCCAGGAGGCTCGTATTAGG |
| | | CAGGGAGGCCGCCGTAATTCTGGGATCAAAAGCGGGAAGGTGCGAACTCCTCTTTGTCTCTGCGTGCCCGGCGCGCCCCCCTCCCGGT |
| | | GGGTGATAAACCCACTCTGGCGCCGGCCATGCGCTGGGTGATTAATTTGCGAACAAACAAAAGCGGCCTGGTGGCCACTGCATTCGGGT |
| | | TAAACATTGGCCAGCGTGTTCCGAAGGCTTGT |
| 53 | chr12 | ATCAACATCGTGGCTTTGGTCTTTTCCATCATGGTGAGTGAATCACGGCCAGAGGCAGCCTGGGAGGAGAGACCCGGGCGGCTTTGAGC |
| | group- | CCCTGCAGGGGAGTCCGCGCGCTCTCTGCGGCTCCCTTCCTCACGGCCCGGCCCGCGCTAGGTGTTCTTTGTCCTCGCACCTCCTCCTC |
| | 00801 | ACCTTTCTCGGGCTCTCAGAGCTCTCCCCGCAATCATCAGCACCTCCTCTGCACTCCTCGTGGTACTCAGAGCCCTGATCAAGCTTCCCC |
| | | CAGGCTAGCTTTCCTCTTCTTTCCAGCTCCCAGGGTGCGTTTCCTCTCCAACCCGGGGAAGTTCTTCCGTGGACTTTGCTGACTCCTCTGA |
| | | CCTTCCTAGGCACTTGCCCGGGGCTTCTCAACCCTCTTTTCTAGAGCCCCAGTGCGCGCCACCCTAGCGAGCGCAGTAAGCTCATACCCC |
| | | GAGCATGCAGGCTCTACGTTCCTTTCCCTGCCGCTCCGGGGGCTCCTGCTCTCCAGCGCCCAGGACTGTCTCTATCTCAGCCTGTGCTC |
| | | CCTTCTCTCTTTGCTGCGCCCAAGGGCACCGCTTCCGCCACTCTCCGGGGGGTCCCCAGGCGATTCCTGATGCCCCCTCCTTGATCCCG |
| | | TTTCCGCGCTTTGGCACGGCACGCTCTGTCCAGGCAACAGTTTCCTCTCGCTTCTTCCTACACCCAACTTCCTCTCCTTGCCTCCCTCCGG |
| | | CGCCCCCTTTTTAACGCGCCCGAGGCTGGCTCACACCCACTACCTCTTTAGGCCTTTCTTAGGCTCCCCGTGTGCCCCCCTCACCAGCAA |
| | | AGTGGGTGCGCCTCTCTTACTCTTTCTACCCAGCGCGTCGTAGTTCCTCCCCGTTTGCTGCGCACTGGCCCTAACCTCTCTTCTCTTGGTG |
| | | TCCCCCAGAGCTCCCAGGCGCCCCTCCACCGCTCTGTCCTGCGCCCGGGGCTCTCCCGGGAATGAACTAGGGGATTCCACGCAACGTG |
| | | CGGCTCCGCCCGCCCTCTGCGCTCAGACCTCCCGAGCTGCCCGCCTCTCTAGGAGTGGCCGCTGGGGCCTCTAGTCCGCCCTTCCGGA |
| | | GCTCAGCTCCCTAGCCCTCTTCAACCCTGGTAGGAACACCCGAGCGAACCCCACCAGGAGGGCGACGAGCGCCTGCTAGGCCCTCGCC |
| | | TTATTGACTGCAGCAGCTGGCCCGGGGGTGGCGGCGGGGTGAGGTTCGTACCGGCACTGTCCCGGGACAACCCTTGCAGTTGC |
| 54 | PAX9 | ACAAATAAAACACCCTCTAGCTTCCCCTAGACTTTGTTTAACTGGCCGGGTCTCCAGAAGGAACGCTGGGGATGGGATGGGTGGAGAGAG |
| | | GGAGCGGCTCAAGGACTTTAGTGAGGAGCAGGCGAGAAGGAGCACGTTCAGGCGTCAAGACCGATTTCTCCCCCTGCTTCGGGAGACTT |
| | | TTGAACGCTCGGAGAGGCCCGGCATCTCACCACTTTACTTGGCCGTAGGGGCCTCCGGCACGGCAGGAATGAGGGAGGGGGTCCGATT |
| | | GGACAGTGACGGTTTGGGGCCGTTCGGCTATGTTCAGGGACCATATGGTTTGGGGACAGCCCCAGTAGTTAGTAGGGGACGGGTGCGTT |
| | | CGCCCAGTCCCCGGATGCGTAGGGAGGCCCAGTGGCAGGCAGCTGTCCCAAGCAGCGGGTGCGCGTCCCTGCGCGCTGTGTGTTCATT |
| | | TTGCAGAGCCAGCCTTCGGGGAGGTGAACCAGCTGGGAGGAGTGTTCGTGAACGGGAGGCCGCTGCCCAACGCCATCCGGCTTCGCAT |
| | | CGTGGAACTGGCCCAACTGGGCATCCGACCGTGTGACATCAGCCGCCAGCTACGGGTCTCGCACGGCTGCGTCAGCAAGATCCTGGCG |
| | | CGATACAACGAGACGGGCTCGATCTTGCCAGGAGCCATCGGGGGCAGCAAGCCCCGGGTCACTACCCCCACCGTGGTGAAACACATCC |
| | | GGACCTACAAGCAGAGAGACCCCGGCATCTTCGCCTGGGAGATCCGGGACCGCCTGCTGGCGGACGGCGTGTGCGACAAGTACAATGT |
| | | GCCCTCCGTGAGCTCCATCAGCCGCATTCTGCGCAACAAGATCGGCAACTTGGCCCAGCAGGGTCATTACGACTCATACAAGCAGCACC |
| | | AGCCGACGCCGCAGCCAGCGCTGCCCTACAACCACATCTACTCGTACCCCAGCCCTATCACGGCGGCGGCCGCCAAGGTGCCCACGCC |
| | | ACCCGGGGTGC |
| 55 | SIX1 | AGGAGGCGCAACGCGCTGCCAGGGCGGCTTTATCCTGCCGCCACAGGGCGGGGACCAGCCCGGCAGCCGGGTGTCCAGCGCCGCTCA |
| | | CGTGCCTCGCCTGGAGCTTAGCTCTCAGACTCCGAAGAGGGCGACTGAGACTTGGGCCTGGGAGTTGGCTTCGGGGTACCCAAGGCGA |
| | | CGACAGCTGAGTTGTACCACGAAGCTCAGGCCGAGGCCTCCTCCCTTGTCTGGCCTTCGAATCCATACTGGCAGCCTCTCCTCTCAGGCA |
| | | CTCCGCGGGCCGGGCCACTAGGCCCCCTGCTCCTGGAGCTGCGCTATGATCCGGGTCTTGAGATGCGCGCGATTCTCTCTGAACCGGT |
| | | GGAGAGGAGGCTCTGCCCCGCGCGGAGCGAGGACAGCGGCGCCCGAGCTTCCCGCGCCTCTCCAGGGCCCAATGGCAAGAACAGCCT |
| | | CCGAAGTGCGCGGATGACAGGAAAAGATCTTCAGTTCTTCTGCCGCTAGAGAAGTGCGGGATACAAGCCTCTATTGGATCCACAACCTGG |
| | | AGTCCTGCCTTCGGA |
| 56 | ISL2 | ATCTGCGTGCCCTTTTCTGGGCGAGCCCTGGGAGATCCAGGGAGAACTGGGCGCTCCAGATGGTGTATGTCTGTACCTTCACAGCAAGG |
| | | CTTCCCTTGGATTTGAGGCTTCCTATTTTGTCTGGGATCGGGGTTTCTCCTTGTCCCAGTGGCAGCCCCGCGTTGCGGGTTCCGGGCGCT |
| | | GCGCGGAGCCCAAGGCTGCATGGCAGTGTGCAGCGCCCGCCAGTCGGGCTGGTGGGTTGTGCACTCCGTCGGCAGCTGCAGAAAGGT |
| | | GGGAGTGCAGGTCTTGCCTTTCCTCACCGGGCGGTTGGCTTCCAGCACCGAGGCTGACCTATCGTGGCAAGTTTGCGGCCCCCGCAGAT |
| | | CCCCAGTGGAGAAAGAGGGCTCTTCCGATGCGATCGAGTGTGCGCCTCCCCGCAAAGCAATGCAGACCCTAAATCACTCAAGGCCTGGA |
| | | GCTCCAGTCTCAAAGGTGGCAGAAAAGGCCAGACCTAACTCGAGCACCTACTGCCTTCTGCTTGCCCCGCAGAGCCTTCAGGGACTGAC |
| | | TGGGACGCCCCTGGTGGCGGGCAGTCCCATCCGCCATGAGAACGCCGTGCAGGGCAGCGCAGTGGAGGTGCAGACGTACCAGCCGCC |
| | | GTGGAAGGCGCTCAGCGAGTTTGCCCTCCAGAGCGACCTGGACCAACCCGCCTTCCAACAGCTGGTGAGGCCCTGCCCTACCCGCCCC |
| | | GACCTCGGGACTCTGCGGGTTGGGGATTTAGCCACTTAGCCTGGCAGAGAGGGGAGGGGGTGGCCTTGGGCTGAGGGGCTGGGTACA |
| | | GCCCTAGGCGGTGGGGGAGGGGGAACAGTGGCGGGCTCTGAAACCTCACCTCGGCCCATTACGCGCCCTAAACCAGGTCTCCCTGGAT |
| | | TAAAGTGCTCACAAGAGAGGTCGCAGGATTAACCAACCCGCTCCCCCGCCCTAATCCCCCCCTCGTGCGCCTGGGGACCTGGCCTCCTT |
| | | CTCCGCAGGGCTTGCTCTCAGCTGGCGGCCGGTCCCCAAGGGACACTTTCCGACTCGGAGCACGCGGCCCTGGAGCACCAGCTCGCGT |
| | | GCCTCTTCACCTGCCTCTTCCCGGTGTTTCCGCCGCCCCAGGTCTCCTTCTCCGAGTCCGGCTCCCTAGGCAACTCCTCCGGCAGCGAC |
| | | GTGACCTCCCTGTCCTCGCAGCTCCCGGACACCCCCAACAGTATGGTGCCGAGTCCCGTGGAGACGTGAGGGGGACCCCTCCCTGCCA |
| | | GCCCGCGGACCTCGCATGCTCCCTGCATGAGACTCACCCATGCTCAGGCCATTCCAGTTCCGAAAGCTCTCTCGCCTTCGTAATTATTCT |
| | | ATTGTTATTTATGAGAGAGTACCGAGAGACACGGTCTGGACAGCCCAAGGCGCCAGGATGCAACCTGCTTTCACCAGACTGCAGACCCCT |
| | | GCTCCGAGGACTCTTAGTTTTTCAAAACCAGAATCTGGGACTTACCAGGGTTAGCTCTGCCCTCTCCTCTCCTCTCTACGTGGCCGCCGCT |
| | | CTGTCTCTCCACGCCCCACCTGTGT |
| 57 | DLX4 | AGGTCTCTTCAGACTGCCCATTCTCCGGGCCTCGCTGAATGCGGGGGCTCTATCCACAGCGCGCGGGGCCGAGCTCAGGCAGGCTGGGGC |
| | | GAAGATCTGATTCTTTCCTTCCCGCCGCCAAACCGAATTAATCAGTTTCTTCAACCTGAGTTACTAAGAAAGAAAGGTCCTTCCAAATAA |
| | | AACTGAAAATCACTGCGAATGACAATACTATACTACAAGTTCGTTTTGGGGCCGGTGGGTGGGATGGAGGAGAAAGGGCACGGATAATCC |
| | | CGGAGGGCCGCGGAGTGAGGAGGACTATGGTCGCGGTGGAATCTCTGTTCCGCTGGCACATCCGCGCAGGTGCGGCTCTGAGTGCTGG |
| | | CTCGGGGTTACAGACCTCGGCATCCGGCTGCAGGGGCAGACAGAGACCTCCTCTGCTAGGGCGTGCGGTAGGCATCGTATGGAGCCCA |
| | | GAGACTGCCGAGAGCACTGCGCACTCACCAAGTGTTAGGGGTGCCCGTGATAGACCGCCAGGGAAGGGGCTGGTTCGGAGGGAATTCC |
| | | CGCTACCGGGAAGGTCGGAACTCGGGGTGATCAAACAAGGAATGCATCTCACCTCCGTGGGTGCTTGTGCTGCGCAAGGAATTATTACC |
| | | GGAGCGGTTGCGATGGCCTTTGCCCGGCGACCCAAGAAGAGTAAGCAAACTACCGTCCACCCAGCGGATCAGGTCCAAT |
| 58 | CBX4 | GATGTCCTGTTTCTAGCAGCCTCCAGAGCCAAGCTAGGCGAGAGGCGTAGGAGGCAGAGAGAGCGGGCGCGGGAGGCCAGGGTCCGC |
| | | CTGGGGGCCTGAGGGGACTTCGTGGGGTCCCGGGAGTGGCCTAGAAACAGGGAGCTGGGAGGGCCGGGAAGAGCTTGAGGCTGAGCG |
| | | GGGGACGAACGGGCAGCGCAAAGGGGAGATGAACGGAATGGCCGAGGAGCCACGCATTCGCCTTGTGTCCGCGGACCCTTGTTCCCGA |
| | | CAGGCGACCAAGCCAAGGCCCTCCGGACTGACGCGGCCTGAGCAGCAGCGAGTGTGAAGTTTGGCACCTCCGGCGGCGAGACGGCGC |
| | | GTTCTGGCGCGCGGCTCCTGCGTCCGGCTGGTGGAGCTGCTGCGCCCTATGCGGCCTGCCGAGGGCGCCGCCGAGGGCCCGCGAGCT |
| | | CCGTGGGGTCGGGGTGGGGGGACCCGGGAGCGGACAGCGCGGCCCGAGGGGCAGGGGCAGGGGCGCGCCTGGCCTGGGGTGTGTC |
| | | TGGGCCCCGGCTCCGGGCTCTTGAAGGACCGCGAGCAGGAGGCTTGCGCAATCCCTTGGCTGAGCGTCCACGGAGAAAGAAAAAGAGC |
| | | AAAAGCAGAGCGAGAGTGGAGCGAGGGATGGGGGCGGGCAAAGAGCCATCCGGGTCTCCACCACCGCCCTGACACGCGACCCGGCTG |
| | | TCTGTTGGGGACCGCACGGGGGCTCGGGCGAGCAGGGGAGGGAGGAGCCTGCGCGGGGCTCGTGTTCGCCCAGGAATCCCGGAGAA |
| | | GCTCGAAGACGGTCTGGTGTTGAACGCACACGTGGACTCCATTTCATTACCACCTTGCAGCTCTTGCGCCACGGAGGCTGCTGCTGCCC |
| | | GGCGGCTGCTACCCACCGAGACCCACGTGGCCCCTCCCCAGGGGTGTAGGGGTGACGGTTGTCTTCTGGTGACAGCAGAGGTGTTGGG |
| | | TTTGCGACTGATCTCTAACGAGCTTGAGGCGCAAACCTAGGATTCCCTGAGTGTTGGGGTGCGGCGGGGGGGCAAGCAAGGTGGGACG |
| | | ACGCCTGCCTGGTTTCCCTGACTAGTTGCGGGGGGTGGGGGCCGGCTCTCAGGGGCCACCAGAAGCTGGGTGGGTGTACAGGAAAATA |
| | | TTTTCTCCTGCCGTGTTTGGCTTTTCCTGGCATTTTTGCCCAGGGCGAAGAACTGTCGCGCGGGGCAGCTCCACCGCGGAGGGAGAG |
| | | GGGTCGCGAGGCTGGCGCGGGAAGCGCTGTAGGTGGCAGTCATCCGTCCACGCCGCACAGGCCGTCTGCGCCGTCGGACCATCGGGA |
| | | GGTCTGCAGCAACTTTGTCCCGGCCAGTCCCCTTGTCCGGGAAGGGGCTGAGCTTCCCGACACTCTACCCTCCCCCTCTTGAAAATCCCC |
| | | TGGAAAATCTGTTTGCAATGGGTGTTTCCGCGGCGTCCAGGTCTGGGCTGCCGGGGGAGGCCGAGCGGCTGCTGCAGCCTCCCTGCTG |
| | | CCAGGGGCGTCGGACTCCGCTTCGCTCACTACGCCCAGGCCCCTCAGGGGCCCACGCTCAGGACTTCGGGGCCACACAGCAGGACCC |
| | | GGTGCCCCGACGACGAGTTTGCGCAGGACCCGGGCTGGGCCAGCCGCGGAGCTGGGGAGGAAGGGGCGGGGGTCGGTGCAGCGGAT |
| | | CTTTTCTGTTGCTGCCTGTGCGGCGGCAGGAAGCGTCTTGAGGCTCCCCAAGACTACCTGAGGGGCCGCCCAAGCACTTCAGAAGCCCA |
| | | AGGAGCCCCCGGCCACCCCCGCTCCTGGCCTTTTTGCCAACGACTTTGAAAGTGAAATGCACAAGCACCAGCAATTGACTTCCCTTCCGT |
| | | GGTTATTTATTTTGTCTTTGTGGATGGTGGGCAGATGGGGAGAGAGGCCCCTACCTAACCTCGGTGGCTGGTCCCTAGACCACCCCTGCC |
| | | AGCCGGTGTGGGGAGGAGCTCAGGTCCGCGGGAGAGCGAATGGGCGCCAGGAGGTGGGACAGAATCCTGGGAAGGTACAGCGGACGC |
| | | CCTGGAAGCTCCCCTGATGCCCCAGAGGGCCCTTCCTGGGAAACCTCCCGGGGGGGTGCCCCATACCATCCCACCCGGCTGTCTTGGC |
| | | CCCTCCCAGGGAGCCGCAGGAGAAACTAGCCCTACACCTGGGATTCCCAGAGCCTTCTGCTGGGGCTCCTGCCCCCGACTTCGGATAAC |
| | | CAGCTCCGCACAGGTCCCCGAGAAGGGCCGCTGGCCTGCTTATTTGATACTGCCCCCTCCCAGACAGGGGCTGGTCGAGCCCCTGGTTC |
| | | TGCTGCCAGACTGAAGCCTTCCAGACGCCACCTCGGTTTGGGCCCCCAGGGCCCTCAGGGGCCCCAGGAGAGGAGAGCTGCTATCTAG |
| | | CTCAGCCACAGGCTCGCTCCTGGTGGGGGCCAGGCTGAAGGAGTGGACCCTGGAGAGGTCGGGAACCTTTTAACAGCCGTGGGCTGGA |
| | | GGGTGGCTACTAAGTGTTCGGTCTGGGAAGAGGCATGACCCGCACCATCCCGGGGAAATAAACGACTTCTTAAGGGAATCTTCTCGCTGA |
| | | GCGGGTGCTCTGGGCCAGGAGATTGCCACCGCCAGCCCACGGAACCCAGATTTGGGCTCTGCCTTGAGCGGGCCGCCTGTGGCTTCCC |
| | | GGGTCGCTCCCCCGACTCAGAAAGCTCTCAAGTTGGTATCGTTTTCCCGGCCCTCGGAGGTGGATTGCAGATCACCGAGAGGGGATTTA |
| | | CCAGTAACCACTACAGAATCTACCCGGGCTTTAACAAGCGCTCATTTCTCTCCCTTGTCCTTAGAAAAACTTCGCGCTGGCGTTGATCA |
| | | TATCGTACTTGTAGCGGCAGCTTAGGGGCAGCGGAACTGGTGGGGTTGTGCGTGCAGGGGGAGGCTGTGAGGGAGCCCTGCACTCCGCCC |
| | | CTCCACCCTTCTGGAGGAGTGGCTTTGTTTCTAAGGGTGCCCCCCCAACCCCCGGGTCCCCACTTCAATGTTTCTGCTCTTTGTCCCACC |
| | | GCCCGTGAAAGCTCGGCTTTCATTTGGTCGGCGAAGCCTCCGACGCCCCCGAGTCCCACCCTAGCGGGCCGCGCGGCACTGCAGCCGG |
| | | GGGTTCCTGCGGACTGGCCCGACAGGGTGCGCGGACGGGGACGCGGGCCCCGAGCACCGCGACGCCAGGGTCCTTTGGCAGGGCCC |
| | | AAGCACCCCT |
| 59 | EDG6 | TGGCGGCCGGCGGGCACAGCCGGCTCATTGTTCTGCACTACAACCACTCGGGCCGGCTGGCCGGGCGCGGGGGGCCGGAGGATGGC |
| | | GGCCTGGGGGCCCTGCGGGGGCTGTCGGTGGCCGCCAGCTGCCTGGTGGTGCTGGAGAACTTGCTGGTGCTGGCGGCCATCACCAGC |
| | | CACATGCGGTCGCGACGCTGGGTCTACTATTGCCTGGTGAACATCACGCTGAGTGACCTGCTCACGGGCGCGGCCTACCTGGCCAACGT |
| | | GCTGCTGTCGGGGGCCCGCACCTTCCGTCTGGCGCCCGCCCAGTGGTTCCTACGGGAGGGCCTGCTCTTCACCGCCCTGGCCGCCTCC |
| | | ACCTTCAGCCTGCTCTTCACTGCAGGGGAGCGCTTTGCCACCATGGTGCGGCCGGTGGCCGAGAGCGGGGCCACCAAGACCAGCCGCG |
| | | TCTACGGCTTCATCGGCCTCTGCTGGCTGCTGGCCGCGCTGCTGGGGATGCTGCCTTTGCTGGGCTGGAACTGCCTGTGCGCCTTTGAC |
| | | CGCTGCTCCAGCCTTCTGCCCCTCTACTCCAAGCGCTACATCCTCTTCTGCCTGGTGATCTTCGCCGGCGTCCTGGCCACCATCATGGGC |
| | | CTCTATGGGGCCATCTTCCGCCTGGTGCAGGCCAGCGGGCAGAAGGCCCCACGCCCAGCGGCCCGCCGCAAGGCCCGCCGCCTGCTG |
| | | AAGACGGTGCTGATGATCCTGCTGGCCTTCCTGGTGTGCTGGGGCCCACTCTTCGGGCTGCTGCTGGCCGACGTCTTTGGCTCCAACCT |
| | | CTGGGCCCAGGAGTACCTGCGGGGCATGGACTGGATCCTGGCCCTGGCCGTCCTCAACTCGGCGGTCAACCCCATCATCTACTCCTTCC |
| | | GCAGCAGGGAGGTGTGCAGAGCCGTGCTCAGCTTCCTCTGCTGCGGGTGTCTCCGGCTGGGCATGCGAGGGCCCGGGGACTGCCTGG |
| | | CCCGGGCCGTCGAGGCTCACTCCGGAGCTTCCACCACCGACAGCTCTCTGAGGCCAAGGGACAGCTTTC |
| 60 | chr13 | TAGTAAGGCACCGAGGGGTGGCTCCTCTCCCTGCAGCGGCTGTCGCTTACCATCCTGTAGACCGTGACCTCCTCACACAGCGCCAGGAC |
| | group- | GAGGATCGCGGTGAGCCAGCAGGTGACTGCGATCCTGGAGCTGGTCGCAGCAGGCCATCCTGCACGCGGTGGAGGCGCCCCCTGCAG |
| | 00005 | GCCGCAGCGCATCCCCAGCTTCTGGACGCACTGTGAGCGGTTATGCAGCAGCACGCTCATATGAGATGCCCCGCAGGGTGCTATGCAGG |
| | | CCCACGTCCCCACAAAGCCCATGGCAGGCGCCCGGGTGCCGGAGCACGCACTTGGCCCCATGGATCTCTGTGCCCAGGGCTCAGCCAG |
| | | GCATCTGGCCGCTAAAGGTTT |
| 61 | CRYL1 | TCTCATCTGAGCGCTGTCTTTCACCAGAGCTCTGTAGGACTGAGGCAGTAGCGCTGGCCCGCCTGCGAGAGCCCGACCGTGGACGATGC |
| | | GTCGCGCCCTTCCCATCGCGGCCTGGGCGGGCCCGCCTGCCCTCGGCTGAGCCCGGTTTCCCTACCCCGGGGCACCTCCCCTCGCCC |
| | | GCACCCGGCCCCAGTCCCTCCCAGGCTTGCGGGTAGAGCCTGTCTTTGCCCAGAAGGCCGTCTCCAAGCT |
| 62 | IL17D | CAGTCCCCGAGGCCCTCCCCGGTGACTCTAACCAGGGATTTCAGCGCGCGGCGCGGGGCTGCCCCCAGGCGTGACCTCACCCGTGCTC |
| | | TCTCCCTGCAGAATCTCCTACGACCCGGCGAGGTACCCCAGGTACCTGCCTGAAGCCTACTGCCTGTGCCGGGGCTGCCTGACCGGGCT |
| | | GTTCGGCGAGGAGGACGTGCGCTTCCGCAGCGCCCCTGTCTACAT |
| 63 | IRS2 | AGAGAGACATTTTCCACGGAGGCCGAGTTGTGGCGCTTGGGGTTGTGGGCGAAGGACGGGGACACGGGGGTGACCGTCGTGGTGGAG |
| | | GAGAAGGTCTCGGAACTGTGGCGGCGGCGGCCCCCCTGCGGGTCTGCGCGGATGACCTTGGCGCCGCGGTGGGGGTCCGGGGGCTG |
| | | GCTGGCCTGCAGGAAGGCCTCGACTCCCGACACCTGCTCCATGAGGCTCAGCCTCTTCACGCCCGACGTCGGGCTGGCCACGCGGGCA |
| | | GCTTCTGGCTTCGGGGGGGCCGCGATAGGTTGCGGCGGGGTGGCGGCCACACCAAAAGCCATCTCGGTGTAGTCACCATTGTCCCCGG |
| | | TGTCCGAGGACAACGATGAGGCGGCGCCCGGGCCCTGGGCGGTGGCAACGGCCGAGGCGGGGGGCAGGCGGTACAGCTCCCCCGGG |
| | | GCCGGCGGCGGTGGCGGCGGCTGCAGAGACGACGACGGGGACGCGGACGGACGCGGGGGCAACGGCGGATACGGGGAGGAGGCCT |
| | | CGGGGGACAGGAGGCCGTCCAAGGAGCCCACGGGGTGGCCGCTCGGGGCGCCCGGCTTAGGAGACTTGGGGGAGCTGAAGTCGAGG |
| | | TTCATGTAGTCGGAGAGCGGAGACCGCTGCCGGCTGTCGCTGCTGGTGCCCGGGGTGCCTGAGCCCAGCGACGAGGCCGGGCTGCTG |
| | | GCGGACAAGAGCGAGGAGGACGAGGCCGCCGACGCCAGCAGGGGAGGCGCGGGCGGCGACAGGCGGGCCCCGGGCTCGCCAAAGT |
| | | CGATGTTGATGTACTCGCCGGGGCTCTTGGGCTCCGGTGGCAGTGGGTACTCGTGCATGCTGGGCAGGCTGGGCAGCCCCTCCAGGGA |
| | | CAGGCGCGTGGGCCTCACCGCCCGGCCGCGCTGGCCCAAGAAGCCCTCCGGGCGGCCGCCGCTAGGCCGCACGGGCGAAGGCACTA |
| | | CAGGGTGAGGGGGCTGCGTGGGGCCGGCCCCGAAGGCGCTGGCCGCCTGGCTGGGCCCTGGCGTGGCCTGAGGCTCCAGACGCTCC |
| | | TCCTCCAGGATGCGCCCCACGGGGGAGCTCATGAGCACGTACTGGTCGCTGTCCCCGCCACAGGTGTAGGGGGCCTTGTAGGAGCGGG |
| | | GCAAGGAGCTGTAGCAGCAGCCGGGAACGCCCCTGAGCGGCTCCCCGCCGGGGTGCAGGGCTGCGGAGAAGAAGTCGGGCGGGGTG |
| | | CCCGTGGTGACCGCGTCGCTGGGGGACACGTTGAGGTAGTCCCCGTTGGGCAGCAGCTTGCCATCTGCATGCTCCATGGACAGCTTGG |
| | | AACCGCACCACATGCGCATGTACCCACTGTCCTCGGGGGAGCTCTCGGCGGGCGAGCTGGCCTTGTAGCCGCCCCCGCTCGCCGGGAA |
| | | TGTCCTGCCCGCCGCAGAGGTGGGTGCTGGCCCCGCAGGCCCCGCAGAAGGCACGGCGGCGGCGGCGGCGGCGGCGGCCCTGGGCT |
| | | GCAAGATCTGCTTGGGGGCGGACACGCTGGCGGGGCTCATGGGCATGTAGTCGTCGCTCCTGCAGCTGCCGCTCCCACTGCCCGCGAG |
| | | GGCCGCGCCGGGCGTCATGGGCATGTAGCCGTCGTCTGCCCCCAGGTTGCTGCTGGAGCTCCTGTGGGAGCCGATCTCGATGTCTCCG |
| | | TAGTCCTCTGGGTAGGGGTGGTAGGCCACCTTGGGAGAGGACGCGGGGCAGGACGGGCAGAGGCGGCCCGCGCTGCCCGAGAAGGTG |
| | | GCCCGCATCAGGGTGTATTCATCCAGCGAGGCAGAGGAGGGCTGGGGCACCGGCCGCTGCCGGGCTGGCGTGGTCAGGGAGTAGGTC |
| | | CTCTTGCGCAGCCCTCGGTCCAGGTCCTGGGCCGCGTCCCCCGAGACCCGGCGGTAGGAGCGGCCACAGTGGCTCAGGGGCCTGTCC |
| | | ATGGTCATGTACCCGTAGAACTCACCGCCGCCGCCGCCGTCTCGGGCCGGGGGCGTCTCCGCGATGGACTCGGGCGTGTTGCTTCGGT |
| | | GGCTGCAGAAGGCGCGCAGGTCGCCTGGGCTGGAGCCGTACTCGTCCAGGGACATGAAGCCGGGGTCGCTGGGGGAGCCCGAGGCG |
| | | GAGGCGCTGCCGCTGGAGGGCCGCTGGCCGGGGCCGTGGTGCAGCGGATGCGGCAGAGGCGGGTGCGGGCCGGGCGGCGGCGGGT |
| | | AGGAGCCCGAGCCGTGGCCGCTGCTGGACGACAGGGAGC |
| 64 | chr13 | TAACCTAAAGAATGAAGTCATGCCCCGGCCTGCACCCGGGAAACTGCACACAGCGAAAGATCGCCACTGAGATAAAGAGCTGAAAGCTAT |
| | group- | TCCCCAATTCAGCTGTTTCAGCCGTGCGGTCTCACAATGGGCTCACAGACGGCAGCATC |
| | 00350 | |
| 65 | MCF2L | GTTTCCACAATCCACCTCGTAGCTGGGGCGTGCCGCTTGCCTCGGCTTGTCCCGGCAGAACACTCTTACCTTTAATGGCGACTGAAAAGT |
| | | TGCCACGAGTTCCTGATCATTGTGGTAGGTGCTGCGTGAAGCTGAGACGTGCGTGAGCCACATCCCAGGGGGCTTTGAGCCCCCACCGC |
| | | GGCGGCGGCTGAGGGGAGGCTTGTCGTACTCGCACAGGAGGACACAGGGCTGCAGTGTTCACTCCAGGGCCTCTTATCATTGGGATCT |
| | | GAGGAATTTTCCGAGAGGAAGTGCGAATTAACAATGATGAAAGGTTTGTGAGTGAGTGACAGGCACGTTCTATTGAGCACTGCATGGGGC |
| | | ATTATGTGCCACCAGAGACGGGGGCAGAGGTCAAGAGCCCTCGAGGGCTGGGAGAGTTCGGAGGATAGAAGTCATCAGAGCACAATGAA |
| | | GCCAGACCCTGCAGCCGCCTTCCCCTTCGGGGGCTTCCTTAGAATGCAGCATTGCGGGGACTGAGCTGTCCCAGGTGAAGGGGGGCCG |
| | | TCACGGTGTGTGGACGCCCCTCGGCTCAGCCCTCTAAGAGACTCGGCAGCCAGGATGGGCTCAAGGCATGAGCCCTCAAAGGAGGTTA |
| | | GGAAGGAGCGAGGGAGAAAAGATATGCTTGTGTGACGTCCTGGCCGAAGTGAGAACAATTGTATCAGATAATGAGTCATGTCCCATTGAG |
| | | GGGTGCCGACAAGGACTCGGGAGGAGGCCACGGAGCCCTGTACTGAGGAGACGCCCACAGGGAGCCTCGGGGGCCCAGCGTCCCGG |
| | | GATCACTGGATGGTAAAGCCGCCCTGCCTGGCGT |
| 66 | F7 | TCCAGCTGCAGCGAGGGCGGCCAGGCCCCCTTCTCCGACCTGCAGGGGTAGCGCGGCCTCGGCGCCGGAGACCCGCGCGCTGTCTGG |
| | | GGCTGCGGTGGCGTGGGGAGGGCGCGGCCCCCGGACGCCCCGAGGAAGGGGCACCTCACCGCCCCCACCCAGAGCGCCTGGCCGTG |
| | | CGGGCTGCAGAGGACCCCTCCGGGGCAGAGGCAGGTTCCACGGAAGACCCCGGCCCGCTGGGGCTTCCCCGGAGACTCCAGAG |
| 67 | chr18 | ACTTACTGCTTCCAAAAGCGCTGGGCACAGCCTTATATGACTGACCCCGCCCCCGAGTCCCAGGCCGCCCCATGCAACCGCCCAACCGC |
| | group- | CCAACCGCCACTCCAAAGGTCACCAACCACTGCTCCAGGCCACGGGCTGCCTCTCCCCACGGCTCTAGGGCCCTTCCCCTCCACCGCAG |
| | 00039 | GCTGAC |
| 68 | C18orf1 | TGCCACACCCAGGTACCGCCCGCCCGCGCGAGAGCCGGGCAGGTGGGCCGCGGATGCTCCCAGAGGCCGGCCCAGCAGAGCGATGG |
| | | ACTTGGACAGGCTAAGATGGAAGTGACCTGAG |
| 69 | CD33L3 | TCGCCAGCGCAGCGCTGGTCCATGCAGGTGCCACCCGAGGTGAGCGCGGAGGCAGGCGACGCGGCAGTGCTGCCCTGCACCTTCACG |
| | | CACCCGCACCGCCACTACGACGGGCCGCTGACGGCCATCTGGCGCGCGGGCGAGCCCTATGCGGGCCCGCAGGTGTTCCGCTGCGCT |
| | | GCGGCGCGGGGCAGCGAGCTCTGCCAGACGGCGCTGAGCCTGCACGGCCGCTTCCGGCTGCTGGGCAACCCGCGCCGCAACGACCTC |
| | | TCGCTGCGCGTCGAGCGCCTCGCCCTGGCTGACGACCGCCGCTACTTCTGCCGCGTCGAGTTCGCCGGCGACGTCCATGACCGCTACG |
| | | AGAGCCGCCACGGCGTCCGGCTGCACGTGACAGGCGAGGCGGCGTGGGAGCGGGTCCCCGGCCTCCCTTCCCGCCCTCCCGCCTGCC |
| | | CCGCCCCAAGGGCTACGTGGGTGCCAGGCGCTGTGCTGAGCCAGGAAGGGCAACGAGACCCAGCCCTCTCCTCTACCCCAGGGATCTC |
| | | ACACCTGGGGGTAGTTTAGGACCACCTGGGAGCTTGACACAAATGCAGAATCCAGGTCCCAGGAAGGGCTGAGGTGGGCCCGGGAATA |
| | | GGCATTGCCGTGACTCTCGTAGAGTGACTGTCCCCAGTGGCTCTCAGACGAAGAGGCGAGAAAGACAAGTGAATGGCAATCCTAAATATG |
| | | CCAAGAGGTGCAATGTGGTGTGTGCTACCAGCCCGGAAAGACACTCGCAGCCCCTCTACCCAGGGGTGCACAGACAGCCCACCAAGTAG |
| | | TGCCTAGCACTTTGCCAGACCCTGATATACAAAGATGCCTGAACCAGGGTCCCGTCCCTAGAGCAGTGGCTCTCCACTCTAGCCCCCACC |
| | | CTGCTCTGCGACAATAATGGCCACTTAGCATTTGCTAGGGAGCCGGGACCTAGTCCAAGCACCCACAAGCATGAATTTGCCAAATCTTTTC |
| | | AGCAACCTCTTAAGGCAACTGCTATCATGATCCTCACTTTACACATGGAGAAGCAGAAGCAGAGATGATAGAATCTTTCGCCCAAGGCCAC |
| | | ATCTGTATTGGGACGGGGGCAGCCTGGCACCCAAGTGCCCATTCCTCCCTTCTGACCAGCCCCCACCCCTCCGGCTCTGGCGTCCAAAG |
| | | GGCTAAGGGGAGGGGTGCCCTTGTGACAGTCACCCGCCTTCTCCCCTGCAGCCGCGCCGCGGATCGTCAACATCTCGGTGCTGCCCAG |
| | | TCCGGCTCACGCCTTCCGCGCGCTCTGCACTGCCGAAGGGGAGCCGCCGCCCGCCCTCGCCTGGTCCGGCCCGGCCCTGGGCAACAG |
| | | CTTGGCAGCCGTGCGGAGCCCGCGTGAGGGTCACGGCCACCTAGTGACCGCCGAACTGCCCGCACTGACCCATGACGGCCGCTACACG |
| | | TGTACGGCCGCCAACAGCCTGGGCCGCTCCGAGGCCAGCGTCTACCTGTTCCGCTTCCATGGCGCCAGCGGGGCCTCGACGGTCGCCC |
| | | TCCTGCTCGGCGCTCTCGGCTTCAAGGCGCT |
| 70 | TNFRS | ATGAACTTCAAGGGCGACATCATCGTGGTCTACGTCAGCCAGACCTCGCAGGAGGGCGCGGCGGCGGCTGCGGAGCCCATGGGCCGCC |
| | F11A | CGGTGCAGGAGGAGACCCTGGCGCGCCGAGACTCCTTCGCGGGGAACGGCCCGCGCTTCCCGGACCCGTGCGGCGGCCCCGAGGGG |
| | | CTGCGGGAGCCGGAGAAGGCCTCGAGGCCGGTGCAGGAGCAAGGCGGGGCCAAGGCTTGAGCGCCCCCCATGGCTGGGAGCCCGAA |
| | | GCTCGGAGC |
| 71 | ZNF236 | TCAGTGTTATGTGGGGAGCGCTAGATCGTGCACACAGTAGGCGTCAGGAAGTGTTTTCCCCAGTAATTTATTCTCCATGGTACTTTGCTAA |
| | | AGTCATGAAATAACTCAGATTTTGTTTTCCAAGGAAGGAGAAAGGCCCAGAATTTAAGAGCAGGCAGACACACAACCGGGCACCCCCAGA |
| | | CCCTGGCCCTTCCAGCAGTCAGGAATTGACTTGCCTTCCAAAGCCCCAGCCCGGAGCTTGAGGAACGGACTTTCCTGCGCAGGGGGATC |
| | | GGGGCGCACTCG |
| 72 | chr18 | GTGGAAACACAACCTGCCTTCCATTGTCTGCGCCTCCAAAACACACCCCCCGCGCATCCGTGAAGCTGTGTGTTTCTGTGTTACTACAGG |
| | group- | GGCCGGCTGTGGAAATCCCACGCTCCAGACCGCGTGCCGGGCAGGCCCAGCC |
| | 00342 | |
| 73 | OLIG2 | TCCACACCTCGGGCAGTCACTAGGAAAAGGGTCGCCAACTGAAAGGCCTGCAGGAACCAGGATGATACCTGCGTCAGTCCCGCGGCTGC |
| | | TGCGAGTGCGCGCTCTCCTGCCAGGGGGACCTCAGACCCTCCTTTACAGCACACCGAGGGCCCTGCAGACACGCGAGCGGGCCTTCAG |
| | | TTTGCAAACCCTGAAAGCGGGCGCGGTCCACCAGGACGATCTGGCAGGGCTCTGGGTGAGGAGGCCGCGTCTTTATTTGGGGTCCTCG |
| | | GGCAGCCACGTTGCAGCTCTGGGGGAAGACTGCTTAAGGAACCCGCTCTGAACTGCGCGCTGGTGTCCTCTCCGGCCCTCGCTTCCCCG |
| | | ACCCCGCACAGGCTAACGGGAGACGCGCAGGCCCACCCCACCGGCTGGAGACCCCGGCACGGCCCGCATCCGCCAGGATTGAAGCAG |
| | | CTGGCTTGGACGCGCGCAGTTTTCCTTTGGCGACATTGCAGCGTCGGTGCGGCCACAATCCGTCCACTGGTTGTGGGAACGGTTGGAGG |
| | | TCCCCCAAGAAGGAGACACGCAGAGCTCTCCAGAACCGCCTACATGCGCATGGGGCCCAAACAGCCTCCCAAGGAGCACCCAGGTCCAT |
| | | GCACCCGAGCCCAAAATCACAGACCCGCTACGGGCTTTTGCACATCAGCTCCAAACACCTGAGTCCACGTGCACAGGCTCTCGCACAGG |
| | | GGACTCACGCACCTGAGTTCGCGCTCACAGATC |
| 74 | RUNX1 | CTGCCCTCGCGGATCTCCCCCGGCCTCGCCGGCCTCCGCCTGTCCTCCCACCACCCTCTCCGGGCCAGTACCTTGAAAGCGATGGGCA |
| | | GGGTCTTGTTGCAGCGCCAGTGCGTAGGCAGCACGGAGCAGAGGAAGTTGGGGCTGTCGGTGCGCACCAGCTCGCCCGGGTGGTCGG |
| | | CCAGCACCTCCACCATGCTGCGGTCGCCGCTCCTCAGCTTGCCGGCCAGGGCAGCGCCGGCGTCCGGGGCGCCCAGCGGCAACGCCT |
| | | CGCTCATCTTGCCTGGGCTCAGCGCGGTGGAAGGCGGCGTGAAGCGGCGGCTCGTGCTGGCATCTACGGGGATACGCATCACAACAAG |
| | | CCGATTGAGTTAGGACCCTGCAAACAGCTCCTACCAGACGGCGACAGGGGCGCGGATCTTCAGCAAGCAGCTCCCGGGAGACCAACATA |
| | | CACGTTCAGGGGCCTTTATTACTGCGGGGGGTGGGGGGGGGCGGGGGTGGTTAGGGGAGGAGGGAGACTAAGTTACTAACAGTCCAGGAG |
| | | GGGAAAACGTTCTGGTTCTGCGGATCGGCCTCTGACCCAGGATGGGCTCCTAGCAACCGATTGCTTAGTGCATTAAAAAGTGGAGACTAT |
| | | CTTCCACGAATCTTGCTTGCAGAGGTTAAGTTCTGTCTTTGGCTGTTAGAAAAGTTCCTGAAGGCAAAATTCTCATACACTTCCTAAAAT |
| | | ATTTATGCGAAGAGTAAAACGATCAGCAAACACATTATTTGGAAGTTCCAGTAGTTAATGCCTGTCAGTTTTTTGCAGGTGAGTTTTGTC |
| | | TAAAGTCCCAACAGAACACAATTATCTCCCGTAACAAGGCCACTTTTATCATGCAAAACTGGCTTCAGTCCCGAAAAGCAAGAGCTGAGA |
| | | CTTCCAAAGGTAGTGCTACTAATGTATGTGCACGTATATATAAATATATACATATGCTCTACTTCATAAAATATTTACAATACAATCTGT |
| | | GGAGAATTTAAACACAACAGAAATCCATTAATGTACGCTGCAGATTTTTTTAAGTAGCCTTGAAAATCAGCTTCAGTAGTTGGAGCAGTG |
| | | CTGAGCTAGAAGTACTTGTCATGTTCTCTGTTCTCTCAATGAATTCTGTCAAAACGCTCAGTGCAGAAAATTCAGCGTTTCAGAGATCTT |
| | | CAGCTAATCTTAAAACAACAATCATAAGAAGGCCCAGTCGATGACACTCAGGGTTCTACAGCTCTCCCACATCTGTGAACTCGGGTTTGG |
| | | GGATGTTGGTTAAGTTTGTGGCTGGTCCTCTGGTTTGTTGGGAGTTGAGCAGCCGCAGAGTCACACACATGCAAACACGCACTCTTCGGA |
| | | AGGCAGCCACTGTCTACATCAGCTGGGTGACTCAGCCCTGACTCGGGCAGCAGCGAGACGATACTCCTCCACCGTCGCCCAGCACCCGCC |
| | | GGTTAGCTGCTCCGAGGCACGAACACCCACGAGCGCCGCGTAACCGCAGCAGGTGGAGCGGGCCTTGAGGGAGGGCTCCGCGGCGCAGAT |
| | | CGAAACAGATCGGGCGGCTCGGGTTACACACGCACGCACATCCTGCCACGCACACTGCCACGCACACGCAACTTCACGGCTCGCCTCGGA |
| | | CCACAGAGCACTTTCTCCCCCTGTTGTAAAAGGAAAACAATTGGGGAAAAGTTCGCAGCCAGGAAAGAAGTTGAAAACATCCAGCCAAGA |
| | | AGCCAGTTAATTCAAAAGGAAGAAAGGGGAAAAACAAAAAAAAACAACAAAAAAAGGAAGGTCCAACGCAGGCCAAGGAGAAGCAGCAGA |
| | | GGTTGACTTCCTTCTGGCGTCCCTAGGAGCCCCGGAAAGAAGTGCCTGGCGGCGCAGGGCCGGGCAGCGTGGTGCCCTGGCTGGGTCCGG |
| | | CCGCGGGGCGCCCGTCCCGCCCGCGCCCGCTGGCTCTATGAATGAGAGTGCCTGGAAATGAACGTGCTTTTACTGTAAGCCCGGCCGGAG |
| | | GAATTCCATTCCCTCAGCTCGTTTGCATAGGGGCGGCCGGCGGCCAATCACAGGCCTTTCCGGTATCAGCCAGGGCGCGGCTCGCCGCCG |
| | | CCGGCTCCTGGAATTGGCCCGCGCGCCCCCGCCGCCGCGCCGCGCGCTACTGTACGCAGCCCGGGCGGGGAGTCGGAGGCCACCCC |
| | | CGCGCCCCGCATCCAAGCCTGCATGCTGGCCCGGGGCCCCGCCCGCGTGCGGACCCCTTTCCGCAGCCACACGCAGGCTTGTGCGGC |
| | | TCCGCGAGTGGCCACGGTCCGGAGACCTGGAAAAAGAAAGCAGGCCCCGCCGGCCCGAGGAGGACCCGGCCGGCGCGCCGCACCCG |
| | | GAGAGGCCCGGCCCCGCGAGCCGCTGCAGGCAGGCGCAGTGGCCGCCACGAGGCTCCCGAACCGGGCTGCAGCCCGCGGACGGCCC |
| | | CAGATCCTGCGCGGCCGCCCAGGGCCAGGCCTCCGCTTCCAGGGCGGGGGTGCGATTTGGCCGCGGGGCCCGGGGGAGCCACTCCG |
| | | CGCTCCTGCACCGTCCGGCTGGCAGCTGCGGCGAAGCGGCGCTGATTCCTTGCATGAGGCCGGACGGCGTCCGCGCGTGCCGTTTGCT |
| | | CTCAGCGTCTTCCCTTGGGTCGGTTTCTGTAATGGGTGTTTTTTACCGCTGCGCCCGGGCCGCGGCTCGATCCCTCCGCGCGTCTCACTT |
| | | GCTGCGTGCGTCAGCGGCCAGCGAAGAGTTTCCTAGTCAGGAAAGACCCCAAGAACGCGCGGCTGGAAGGAAAGTTGAAAGCAGCCAC |
| | | GCGGCTTGCTCCCGGGCCTTGTAGCGCCGGCACCCGCAGCAGCCGGACAGCCTGCCCGGGCCCCGCGTCTCCCCTCCGGCTCCCCGG |
| | | AAGCGGCCCCCGCTCCTCTCCCCGCCCCCGTGCGCTCGAGCGGCCCCAGGTGCGGAACCCACCCCGGCTTCGCGTGCGGGCGGCCGC |
| | | TTCCCCCTGCGCCGGTCCCCGCGGTGCTGCGGGCATTTTCGCGGAGCTCGGAGGGCCCCGCCCCCGGTCCGGCGTGCGCTGCCAACT |
| | | CCGACCCCGCCCGGCGGGGCTCCCTCCCAGCGGAGGCTGCTCCCGTCACCATGAGTCCCTCCACGCCCTCCCTGCCGGGCCCTGCAC |
| | | CTCCCGGGGCCTCTCATCCACCCCGGGGCTGCAACCCAGTCCCCGGATCCCGGCCCCGTTCCACCGCGGGCTGCTTTGTGGTCCCCGC |
| | | GGAGCCCCTCAATTAAGCTCCCCGGCGCGGGGGTCCCTCGCCGACCTCACGGGGCCCCTGACGCCCGCTCCTCCCTCCCCCAGGGCTA |
| | | GGGTGCTGTGGCCGCTGCCGCGCAGGGACTGTCCCCGGGCGTTGCCGCGGGCCCGGACGCAGGAGGGGGCCGGGGTTGACTGGCGT |
| | | GGAGGCCTTTCCCGGGCGGGCCCGGACTGCGCGGAGCTGTCGGGACGCGCCGCGGGCTCTGGCGGACGCCAGGGGGCAGCAGCCGC |
| | | CCTCCCTGGACGCCGCGCGCAGTCCCCGGAGCTCCCGGAACGCCCCCGACGGCGCGGGGCTGTGCGGCCCGCCTCGTGGCCTTCGG |
| | | GTCGCCCGGGAAGAACTAGCGTTCGAGGATAAAAGACAGGAAGCCGCCCCAGAGCCCACTTGAGCTGGAACGGCCAAGGCGCGTTTCC |
| | | GAGGTTCCAATATAGAGTCGCAGCCGGCCAGGTGGGGACTCTCGGACCAGGCCTCCCCGCTGTGCGGCCCGGTCGGGGTCTCTTCCCG |
| | | AAGCCCCTGTTCCTGGGGCTTGACTCGGGCCGCTCTTGGCTATCTGTGCTTCAGGAGCCCGGGCTTCCGGGGGGCTAAGGCGGGCGGC |
| | | CCGCGGCCTCAACCCTCTCCGCCTCCGCTCCCCCTGGGCACTGCCAGCACCCGAGTTCAGTTTTGTTTTAATGGACCTGGGGTCTCGGA |
| | | AAGAAAACTTACTACATTTTTCTTTTAAAATGATTTTTTTAAGCCTAATTCCAGTTGTAAATCCCCCCCTCCCCCCGCCCAAACGTCCAC |
| | | TTTCTAACTCTGTCCCTGAGAAGAGTGCATCGCGCGCGCCCGCCCGCCCGCAGGGGCCGCAGCGCCTTTGCCTGCGGGTTCGGACGCGGC |
| | | CCGCTCTAGAGGCAAGTTCTGGGCAAGGGAAACCTTTTCGCCTGGTCTCCAATGCATTTCCCCGAGATCCCACCCAGGGCTCCTGGGGC |
| | | CACCCCCACGTGCATCCCCCGGAACCCCCGAGATGCGGGAGGGAGCACGAGGGTGTGGCGGCTCCAAAAGTAGGCTTTTGACTCCAGG |
| | | GGAAATAGCAGACTCGGGTGATTTGCCCCTCGGAAAGGTCCAGGGAGGCTCCTCTGGGTCTCGGGCCGCTTGCCTAAAACCCTAAACCC |
| | | CGCGACGGGGGCTGCGAGTCGGACTCGGGCTGCGGTCTCCCAGGAGGGAGTCAAGTTCCTTTATCGAGTAAGGAAAGTTGGTCCCAGC |
| | | CTTGCATGCACCGAGTTTAGCCGTCAGAGGCAGCGTCGTGGGAGCTGCTCAGCTAGGAGTTTCAACCGATAAA |
| 75 | AIRE | TTCGGAAGTGAGAGTTCTCTGAGTCCCGCACAGAGCGAGTCTCTGTCCCCAGCCCCCAAGGCAGCTGCCCTGGTGGGTGAGTCAGGCCA |
| | | GGCCCGGAGACTTCCCGAGAGCGAGGGAGGGACAGCAGCGCCTCCATCACAGGGAAGTGTCCCTGCGGGAGGCCCTGGCCCTGATTG |
| | | GGCGCCGGGGCGGAGCGGCCTTTGCTCTTTGCGTGGTCGCGGGGGTATAACAGCGGCGCGCGTGGCTCGCAGACCGGGGAGACGGG |
| | | CGGGCGCACAGCCGGCGCGGAGGCCCCACAGCCCCGCCGGGACCCGAGGCCAAGCGAGGGGCTGCCAGTGTCCCGGGACCCACCGC |
| | | GTCCGCCCCAGCCCCGGGTCCCCGCGCCCACCCCATGGCGACGGACGCGGCGCTACGCCGGCTTCTGAGGCTGCACCGCACGGAGAT |
| | | CGCGGTGGCCGTGGACAG |
| 76 | SUMO3 | ACGCACACTGGGGGTGTGATGGAAAGGGGGACGCGATGGATAGGGGTGGGCGCACACTGGGGGACGCGACGGGGAGGGGTGAGCAC |
| | | ACACTGGGGGTGTGATGGAGAGGGCGACGCAATAGGGAGGGGTGGGCGCACACCAGGGACGCGATGATGGGGACGGGTGGGCGCAC |
| | | ACCAGGTGGCATGATGGGGAGGAGTGGGTACACACCATGGGGGGCGTGATGGGGAGGCGTGGGCGTACACCGGGGGGCGCGATGGG |
| | | GAGGGGTGGGCGCACACCGGGGGACGCGATGGAGGCGGTGGGTGCACACGGGGCGCGATGGGTGGGAGTAGGTGCACACTGAGGGC |
| | | ACGATTGGGGAGACACGAAGGAGAGGGGTGGGCGCACACTGGGGGACGCGATGGCCGGGACACGATGCGGAGAAGTGGGTGAATACC |
| | | GGGGTCGCGATGGGCGCCCTGGAAGGACGGCAGTGCTGCTCACAGGGGCCAGGCCCCTCAGAGCGCGCCCCTTGGGGGTAACCCCAG |
| | | ACGCTTGTTCCCGAGCCGACTCCGTGCACTCGACACAGGATC |
| 77 | C21orf70 | CCACAGGGTGGGGTGCGCCCACCTGCCCTGTCCATGTGGCCTTGGGCCTGCGGGGGAGAGGGAATCAGGACCCACAGGGCGAGCCCC |
| | | CTCCGTAGCCCGCGGCACCGACTGGATCTCAGTGAACACCCGTCAGCCCATCCAGAGGCTAGAAGGGGGA |
| 78 | C21orf123 | TTGAGGTCTCTGTGCATGCTTGTGCGTACCCTGGACTTTGCCGTGAGGGGTGGCCAGTGCTCTGGGTGCCTTTGCCAGACAACTGGTCT |
| | | GCCGGGCCGAGCATTCATGCTGGTC |
| 79 | COL18A1 | TGACGCGCCCCTCTCCCCGCAGCTCCACCTGGTTGCGCTCAACAGCCCCCTGTCAGGCGGCATGCGGGGCATCCGCGGGGCCGACTTC |
| | | CAGTGCTTCCAGCAGG |
| 80 | PRRT3 | AACACACTGTCTCGCACTAGGTGCTCGCGGAAGAGCGCGGCGTCGATGCTGCGGCTCAGGTTGATGGGCGATGGCGGCCGCAGATCCA |
| | | GCTCGCTCAGCGATGGCGCCGGTCCCACACCGTTGCGGGACAGTCCCGGGCCACCCTGGGGTCCGCGACCCAACGACGCAGCCGAGC |
| | | CCCAGGCGCCTGAACTGGGCGTGGCCAGCTGCCCACTCTCCGCCGGGTTGCGGATGAGGCTCTTGCTGATGTCCAAGCTGCCTGCACC |
| | | AACGTTGCTGGGCCCTGCATAGCAGTTATTGGGTCGCTCCGGCACCTCGCTCTTTCCTGACGGCGCCGGGCACGCCAGACGCATCAGCT |
| | | TAGCCCAGCAAGCGTGCTCCGTGGGCGGCCTGGGTCTCGCGGCAGCCACCGCGGCCAACGCCAGGGCGAGCGCCCATGTCAGCTCCA |
| | | GGAGGCGCAGCCAGAAGTGGACACCCCACCAGGCCCACGAGAAGCGGCCCACGCGGCCTGGGCCCGGGTACAGCCAGAGCGCAGCC |
| | | GCCAGCTGCAAGCCGCTAGCCAGCAGCCCCAGCGCGCCCGCCACAGCCAACAGCCGAGGGCCCGGGCTGGCATCCCAGCCCCGTGGG |
| | | CCGTCCAGCAGGCGGCGACGGCACAGGCAGAGCGTGCCCAGAGCCAC |
| 81 | MGC29506 | GTCTGCACGAAGCCCGCGGCGGCCTGCAGGGGGCCCAGCGACTCGTCCAGGGAACCGGTGCGCAGGAGCAGCCGGGGGCGCGGCGC |
| | | GCCGGCCGCCCTTGGGGGACTCTGGGGCCGGGGGCGCAGCTCGATCTGACGCTTGGGCACTGTCCGGGGCCTGGCGGGCGCGGCGC |
| | | CCTCCTCCAGAGCCACCTCCACACACTCGAACTGCGCTGGGGCGGCAGGACTTGGCCCACGGGGCCGCAGCTCTAGGTAGGTGGCCCA |
| | | GCGGGAGCCACCATCGGGGACCTGGGACTGGCGTGGGACCGCGGCGGGAGACGCTGGCCCCGGCGGCAAGGGGCTGATGAAGGCCG |
| | | GCTCCGTGAACTGTTGTTGCGCCTCGCGATCGTCTGCGCCGGAGCAGCCGAACAGGGGTCCGACGCCGAAGATGACTTCCATCTCCCCC |
| | | GACGGCAGCGTGCGCAGCTGGGGCTGGGGTGGCCGTGGGCCGGAACCTGGGCCTCGCGGGAAACCCGAGCCGGGCCCGTGCCGCTG |
| | | GCGGCTATTCTGGGCGCTGACGGACAGGCGAGGCTGCGCGCCCGCCCCCCGCCCAGGAGCCACCCAGGGCCAATTCGCTGGGCCTTT |
| | | CGCGTCCGGCCCAACGTCCGGGGGCTCCGGAGAACCTGGAGCCGTGTAGTAGGAGCCTGACGAACCGGAGGAGTCCTGGCGCCGCGC |
| | | GGGGGCCGTGGGCAGCTGCCTCGGGATCCCAGGCAGGGCTGGCGGGGCGAGCGCGGTCAGCATGGTGGGGCCGGACGCCGTGCACT |
| | | ATCTCCCTCGCATTCGCCTCCGCTGGTGGCGC |
| 82 | TEAD3 | CTGGAGAGAACTATACGGGCTGTGGGAGTCACCGGGCGACTATCACCGGGCCTCCTTTCCACATCCTCCTCCGGGAAGGGACCCCGTTC |
| | | CGGGCCTCGACCGGCGCAGACTGGGCTGACCCACTTTCTTGGGCCCACTGAGTCACCTCGAAACCTCCAGGCCGGTAGCGGGGAGGAG |
| | | AGGAGGAGCAGGCGGGGGTGCCAAGGTGTGGGCTGCGCCCTGGTTAGGGGGCGAGCCCGGCTTGTTTATGAGGAGGAGCGCGGAGGA |
| | | GGATCCAGACACACAGGCTTGCGCGCCCAGACTCGCCCGGCCAGCGGCTGGCGGCCTCCGACGTCACCAAACCGGTTGGGTGAGAGG |
| | | GCAGAGAGCAGGGGGAAGGGCCGCAGTCCCGCCCGCGCCCCCCGGCACGCACCGTACATCTTGCCCTCGTCTGACAGGATGATCTTCC |
| | | G |
| 83 | chr12 | GAGTGCGGAGTGAAGGGGTGCACTGGGCACTCAGCGCGGCCCTTGGGAGGCAGGGCCGCCCCAGCCTGCCCTCCTGTCTGGGAAGGC |
| | group- | CGTCCAGAAGCAGGAGCCCCGGGGAAAACAACTGGCTGGACGGGGCGGCCTTCAGTGTCTCTCCCAGCCTGAGAGTCGCTTCCCACCA |
| | 00022 | CCTGGGCACGAACCTGCTCTGCGATCTCCGGCAAGTTCCTGCGCCTCCTGTCGGTAAAATGCAGATCGTGGCGTCTT |
| 84 | CENTG1 | TCTTCTTTCCGCCCCTAGGGGGCACAAGCGGGCATGTCCAAGCGCCTAGGAGCCCGTACCGCTGGGGACCTCCCCTTCCGCGAACCCC |
| | | GAGCGGGTAGACCCAGAGCAATCCGAGTGTGGAAACAATGGAGAGGGGGCGTGTTGAGCTGGGGTCTCCATGCCTCGTTGGGGAGAGG |
| | | GAGGTGAGTTTGTGTCTTCTGGAAGGCGTGGGGGCTGTGCCCTCGTGGGGGTAGGAAGTGCTCCCGTGGGGCGGGGTGCGGATCGGA |
| | | GAGGTGAGTGGGTGCGTCTGTCCAGCGGTCCGCCCGGTGTGGTCGTGCCCGGCCCGCGTGGGGATGGGGGTGTCTCTCCCGCTGGGC |
| | | AACTATACCAGCGCAACCGGGGCGTCGGCGCGGCCCACGCTAGCGGCGCTGCTCCGGCGGCGGGGGCTGGGCGTGGCGGTGATGCT |
| | | GGGCGTGGTGGCCGCGCTGGGCGTGGTGGCCGCGCTGCCGCCCTCACCCGGGCAGCCGTGCTGGAGAAGGATGTCGGCGCACAGCT |
| | | GGCTTCCAGCCTGGCGGGCGTAGAACAGCGCCGTGCGGCCCTGGGCGTCACGGGCCGCCACGTCCGCGCCGTACTAGAGGGCGGAAA |
| | | CGGCCGCGTGACCGCGCGTCCCCAGGGCGCCCACACCCGGCGCCGCCTCCCCCACATGGCCAAGCCTACTTCCGGGGTCCCTCTGGG |
| | | AATTTCGGGCTTTCCCGCGCCAGGCGTTTTCCGAGATGAAGCCTCAAAGACCCCCTTTCCTCCCCCCAGCTCACGTACCCACAGCAGCAG |
| | | TTGCGTGATGACGACGTGGGCGAGCTCGGCCGCCAGGTGGAGTGGGGAGCGCAGCTGTGGGTCCTCTACGCTGGTGTCGAGCGGCCC |
| | | GTGTCGCGCATGGGCCAAAAGCAGGAGAACGGTAGCCACGTCCTGGGCCTGCACGGCGGCCCACAGCTGGCGGCCCAGCGGCTCCTC |
| | | CGAGGTGCTCAGCGGCGCCAGGAACAGTAGCTGCTCGTACTTGGCGCGAATCCACGACTCGCGCTCCTCCCTGCAAGACCAGGGATCAA |
| | | CGGAAAAGGCTCTAGGGACCCCCAGCCAGGACTTCTGCCCCTACCCACGGGACCGTCTCAGGTTCGCACACCCTCAGCAACCCTCCCCC |
| | | CGCTCTGTTCCCTCACGCTTACCGCGAAGAGTCCCGCGAGGGCTTGGCACGGCCTCGCGTGTCGCTTTCCCACACGCGGTTGGCCGTGT |
| | | CGTTGCCAATAGCCGTCAGCACCAGGGTCAGCTCCCGTGGCCAGTCGTCCAAGTCCAGCGAGCGAACGCGGGACAGGTGTGTGCCCAG |
| | | GTTGCGGTGGATGCCAGAACACTCGATGCAGATGAGGGCGCCCAGGTTCAAGCTGGCCCACGTGGGGTCTGCGGAAGGAGCGTAGAGG |
| | | TCGGCTCCCAGCCGGGCAGCACAGGCACCCCGGCATTCACTACACTCCCTAGCCCCTCCGCTGCCTCCTGGCACTCACTGGGGGCCCC |
| | | GCAGTCCACGCAGATTGAATTCCCCTTGGCGTTCCGGATCGCCTGGAT |
| 85 | CENTG1 | AGCCAGGTCCAGCCCCCGCGCCTGACACCGGCCGGACGTTCCCGGGGCGCCGCAGCTGCGGCGGGAACTCTGGGATCCGGAGCCATC |
| | | TGCTCCCACCCGCTCCGGAGCCAAACCCCGGGGGCCGCCTCCGCTCCCGGACCCGCCTCCTCTCCCGGGAGTGTGAGCCGAACCAAGAGT |
| | | CTCCTGCCTATCTCCTCCAGTAGGAAAATAGTAATAATAATAGACACCCTGCCCCCGTAAAAAACACTACCTTCCCCGTACCGCCTCCC |
| | | AAGTCTCCCGGGGTACGGATTGCCTTTGCAGCAGTTCCGCCCCACCTGACTCACTCCAGGGTCAGCCCCGGGTGGGTTTCAATGCGGCT |
| | | CTGGGGAGGGGGTGGGCAGTGGGGGAAGTGAGGCTTCCTATCCGCCCCCTCTCACTTCACATTTAAATATTCTGCACGTTCCAGCCCCC |
| | | GCGGACTCGCGTACCGCCCAATCCGCCTTCACCGCACGAAAAACATCACTAGCCTGCTCTCAGCCCAGGGGACGACTAGTCCCTGGCGA |
| | | GAAGCTGCCTGCAAGGTCACTGTCATGCCACCTGCCCCAAGTGCTCAGGGGAAACTGAGGCTTCCTCATCCCCTTCACCTTCAACGTCGC |
| | | TCTAAACACGGCAAAGCCCCGTTTCCATGCTCCCAGAGTTCAGCTGAGGCTGGAAGTGGGGTCCTGGGCTTCTCTGGGAGCAATTTTCTA |
| | | GTCACTCTGATCAAGGACGTTACTTTCCCAGAAAGCTCTGAGGCTGAGTCCCTCTGAAATCAAGTCCTTTCTCCTGTCGCACAATGTAGCT |
| | | ACTCGCCCCGCTTCAGGACTCCTATTCTTTGCCCCAATCCTTGACAGAGGGGTGAGCTTGGTTCATCCGCCCACCCCAGAGAAAAGCTTC |
| | | CCTAGTTTCCTGGACCTCGCTCCTCCACCCCAAGCTGAGCATTCCAGGTACCCTTCCCTCCCTGTTCTCAAGCCCTGACTCAACTCACTAG |
| | | GGGAAGCGCGGAGCTCGGCGCCCAGCAGCTCCCTGGACCCGCTGCCAGAAGACAGGCTGGGGGGTCCGGGAAGGGGCCCGGAGCCA |
| | | GGAGGCCCTCCTGTGCTCTTGGTGAAGATGCCGCTGATAAACTTGAGCATCTTGCGGTCACGAGTGGATGCTCGGCCCCCCTCCCGGCC |
| | | CCGTTTCAGCCCCGGAGCTGGAGGCTCCAGAGTGATTGGAGGTGCAGGCCCGGGGGGCTGCGCGGAAGCAGCGGTGACAGCAGTGGC |
| | | TGGACTCGGAGTTGGTGGGAGGGTTAGCGGAGGAGGAGAGCCGGCAGGCGGTCCCGGATGCAAGTCACTGTTGTCCAAGGTCTTACTC |
| | | TTGCCTTTCCGAGGGGACAACTTCCCTCGGGCTCCAGCCCCAGCCCCGACCCCACCAGAGGTCGAAGCTGTAGAGCCCCCTCCCCCGG |
| | | CGGCGGCGGCGGTGGCGGCGGCAGAGACCGAAGCTCCAGTCCCGGCGCTGCTCTTTGACCCCTTGACCCTGGGCTTGCCCTCGCTTTC |
| | | GGGCCATGACAGGCGGCTACCCGCGCCCTTGCCCCCGCCGGCTTTGGCTCCACTCGTGGTCACGGTCTTGCAAGGCTTGGGAGCCGGC |
| | | GGAGGAGGCGCCACCTTGAGCCTCCGGCTGCCGGTGCCAGGGTGCGGAGAGGATGAGCCAGGGATGCCGCCGCCCGCCCGGCCTTCG |
| | | GGCTCCGGGCCGCCCCAGCTCGGGCTGCTGAGCAGGGGGCGCCGGGAGGAGGTGGGGGCGCCCCCAGGCTTGGGGTCGGGGCTCAG |
| | | TCCCCCGGAGAGCGGGGGTCCCGGAGGGACGGCCCAGAGGGAGAGGCGGCGGCCGGGAGCGGGGGAGACTGGGCGGGCCGGACTG |
| | | GCCGGAGCCGGGGACAGGGCTGGGGGCTCCGCGCCCCCGGTGCCCGCGCTGCTCGTGCTGATCCACAGCGCATCCTGCCGGTGGAAG |
| | | AGACGTTCGTGCCGCTTCTTGCCCGGCTCCTCCGCGCCTCGGGGGCTGCCAGGATCCCCAGTCTCGGAGCCTCTGGCACCGGCGGCGC |
| | | CGGCCGCGGCCGCAGACGGAGAAGGCGGCGGCGGAGGCACCGACTCGAGCTTAACCAGGGTCAGCGAGATGAGGTAGGTCGTTGTCC |
| | | GGCGCTGAAGCGCGCCCGCGCCCCGGCTCATGGGGCCCGGAGACCCCCGAGCTGGGGAGGGGAGGGGACTCCCCCGGACTGCCTCA |
| | | GGGGGGCCCGGCCATGGGGCCGCCCTGCTCGCTGCCCCCAGCCCCCGGACCCCGCTGAGCCCCCGGCCCGGCTCCGCTGTCGCCGC |
| | | CGCCTCCGCCGCCTCCGCTTGCGCCCCCCTCCCATCACATGGGGCGCCCCCTCCCCATGCTCCCCGCCCTGCGCCCCCACCCTCTTGG |
| | | AGCCCCGGGACCTTGGTGCTGCTCCAGGGAGGCGCGCCGGACCGTCCACCCCGGCCTGGGTGGGGGCGCTGAGATGGGTGGGGGAG |
| | | GGCGGGGAGGACAGTAGTGGGGGCAAATGGGGGAGAGAGAGGAAAAGGGAGCAGAAAAGGGGACCGGAGGCTAGGGGAAACGAACCT |
| | | GTGCGGGGGAGGCAGGGGCGGGGAATTGGGACTCAAGGGACAGGGGCCGCGGATGCGGTCGGAAAGAGGGTCTAGAGGAGGGTGGG |
| | | AAGCTAGTGG |
| 86 | chr18 | AGGAGCGCAAGGCTTGCAGGGCATGCTGGGAGAGCGCAGGGAACGCTGGGAGAGCGCGGGAAATACTGGGATTGGCTCCCGAGGGCT |
| | group- | GTGAGGAGGGCACGAGGGGACACTCCGATGAAGGCAGGGCACGCGGGGCGAGCCGGGAGCGTCTCCTGAGGGCAGCGAGGAGGGAG |
| | 00304 | CTGAGGCACGCGGGCTCTCAATCGACGCCCCACAGAGACCAAGAGGCCTGGCCTTGGGGGGCAGCTGCTTGAAGGAGGCAGAGCGGA |
| | | AGCGAGGGAGACTGCTGGAGGCCCTGCCGCCCACCCGCCCTTTCCTCCCCCTGAGGAGACGCCTGACGCATCTGCAGTGCAGGAGGCC |
| | | GTGGGCGTTAGAAGTGTTGCTTTTCCAGTTTGTAAGACCATTTTCCTGATTCTCTTCCCCACGGTTGCGGAGGAGCAGGTCAGGGCCGCC |
| | | ATGAGGGCAGGATC |
| 87 | TSHZ1 | TCGACCGCTACTATTATGAAAACAGCGACCAGCCCATTGACTTAACCAAGTCCAAGAACAAGCCGCTGGTGTCCAGCGTGGCTGATTCGG |
| | | TGGCATCACCTCTGCGGGAGAGCGCACTCATGGACATCTCCGACATGGTGAAAAACCTCACAGGCCGCCTGACGCCCAAGTCCTCCACG |
| | | CCCTCCACAGTTTCAGAGAAGTCCGATGCTGATGGCAGCAGCTTTGAGGAGGC |
| 88 | CTDP1 | TGTGCCGTCGCACACAGACGCCCTCAACGTCGGAGAGCTGTGAGCGGGGCCGTGCTCTTGGGATGGGAGCCCCCGGGAGAGCTGCCC |
| | | GCCAACACCACTCCGACGTGATCCATGCTGGACATAAAGTGCTCTTCCCTCCGCTAGTCATCGGCCGAGCGGGCCCCTCGCTCCTGGGT |
| | | GTAAGTTCTTTCTGTGCGTCCTTCTCCCATCTCCGTGCAGTTCAG |
| 89 | KCNG2 | CCATGCGCCGCTGCGCGCGCGAGTTCGGGCTGCTGCTGCTGTTCCTCTGCGTGGCCATGGCGCTCTTCGCGCCACTGGTGCACCTGGC |
| | | CGAGCGCGAGCTGGGCGCGCGCCGCGACTTCTCCAGCGTGCCCGCCAGCTATTGGTGGGCCGTCATCTCCATGACCACCGTGGGCTAC |
| | | GGCGACATGGTCCCGCGCAGCCTGCCCGGGCAGGTGGTGGCGCTCAGCAGCATCCTCAGCGGCATCCTGCTCATGGCCTTCCCGGTCA |
| | | CCTCCATCTTCCACACCTTTTCGCGCTCCTACTCCGAGCTCAAGGAGCAGCAGCAGCGCGCGGCCAGCCCCGAGCCGGCCCTGCAGGA |
| | | GGACAGCACGCACTCGGCCACAGCCACCGAGGACAGCTCGCAGGGCCCCGACAGCGCGGGCCTGGCCGACGACTCCGCGGATGCGCT |
| | | GTGGGTGCGGGCAGGGCGCTGACGCCTGCGCCGCCCAC |

TABLE 4B

| SEQ | | |
|---|---|---|
| ID | GENE | |
| NO | NAME | SEQUENCE |
| 90 | TFAP2E | GTCCTAACATCCCAGGTGGCGGCGCGCTGGCTCCCTGGAGCGGGGCGGGACGCGGCCGCGCGGACTCACGTGCACAACCGCGCGGGA |
| | | CGGGGCCACGCGGACTCACGTGCACAACCGCGGGACCCCAGCGCCAGCGGGACCCCAGCGCCAGCGGGACCCCAGCGCCAGCGGGAC |
| | | CCCAGCGCCAGCGGGACCCCAGCGCCAGCGGGACCCCAGCGCCAGCGGGACCCCAGCGCCAGCGGGTCTGTGGCCCAGTGGAGCGAG |
| | | TGGAGCGCTGGCGACCTGAGCGGAGACTGCGCCCTGGACGCCCCAGCCTAGACGTCAAGTTACAGCCCGCGCAGCAGCAGCAAAGGGG |
| | | AAGGGGCAGGAGCCGGGCACAGTTGGATCCGGAGGTCGTGACCCAGGGGAAAGCGTGGGCGGTCGACCCAGGGCAGCTGCGGCGGCG |
| | | AGGCAGGTGGGCTCCTTGCTCCCTGGAGCCGCCCCTCCCCACACCTGCCCTCGGCGCCCCCAGCAGTTTTCACCTTGGCCCTCCGCGGT |
| | | CACTGCGGGATTCGGCGTTGCCGCCAGCCCAGTGGGGAGTGAATTAGCGCCCTCCTTCGTCCTCGGCCCTTCCGACGGCACGAGGAACT |
| | | CCTGTCCTGCCCCACAGACCTTCGGCCTCCGCCGAGTGCGGTACTGGAGCCTGCCCCGCCAGGGCCCTGGAATCAGAGAAAGTCGCTCT |
| | | TTGGCCACCTGAAGCGTCGGATCCCTACAGTGCCTCCCAGCCTGGGCGGGAGCGGCGGCTGCGTCGCTGAAGGTTGGGGTCCTTGGTGC |
| | | GAAAGGGAGGCAGCTGCAGCCTCAGCCCCACCCCAGAAGCGGCCTTCGCATCGCTGCGGTGGGCGTTCTCGGGCTTCGACTTCGCCAGC |
| | | GCCGCGGGGCAGAGGCACCTGGAGCTCGCAGGGCCCAGACCTGGGTTGGAAAAGCTTCGCTGACTGCAGGCAAGCGTCCGGGAGGGGC |
| | | GGCCAGGCGAAGCCCCGGCGCTTTACCACACACTTCCGGGTCCCATGCCAGTTGCATCCGCGGTATTGGGCAGGAAATGGCAGGGCTGA |
| | | GGCCGACCCTAGGAGTATAAGGGAGCCCTCCATTTCCTGCCCACATTTGTCACCTCCAGTTTTGCAACCTATCCCAGACACACAGAAAGCA |
| | | AGCAGGACTGGTGGGGAGACGGAGCTTAACAGGAATATTTTCCAGCAGTGA |
| 91 | LRRC8D | CACCTTCCCCGAGGTAATTATTTTCTGGGGGGTAGGGGTGGGGGTTGGGAGGGTGAAGAAAGGAAGAAAAAGAAGGCCGATCACACTGG |
| | | GCACCGGCGGAGGAAGCGTGGAGTCCATTGATCTAGGTACTTGTGGGGAGGGGAGAACCCGAGCAGCAGCTGCAAACGGAAGGGCTGTG |
| | | AGCGAGCGGGCGGGCGGGTGGCTGGCAGCGAGGCCACCAGCAGGGGGGGCCCGGGCCGAGGCCGCGCCACCTCGGCACCACGCGGG |
| | | CAGCCGGTGCGGCGGGGTCGCCACGGCCAGGGGAGCGCTGGGTGCCCACCATGGCAGTTATGCAAGCGGTGACCCCCTGGTCTTGCCT |
| | | CCCCGCCGCCCTGCACTCCTTCCTCCCCGCTGCCGACACTTGGATCTCTCTAGCTCTTTCTCTCCCCTGTGTTTTCAAACAGGAAGTGCAC |
| | | GGCTGTCTATAACGTGCTGCCGGGTCTCAGGATGGAGGAGTGAAGTCTCCTGTCGCCGTGGTTCCAGCCTCCGGAGCTCGCCCAAGCCG |
| | | CGTCCCCAGAGAGCGCCCTGAGAGAACAGGGTGGCCGCTTGGTCCAGGTGCGCGGGGTCGGGTCTGGGTCCAGGGAGCGGGTCGGGAA |
| | | GTCTGCGGCACGGAGCACTGCTAGTGTCGGATCTGCATCTCCAGCTCTGTGCTGCAGCTTCACTTGCCCGCCCCCCACCACTGGCTTCTC |
| | | ACCCGGGGTCTCTGCCAAACTCTGGCTGCTGCCGCCCTGGGTTCGGGCCGGCGGAAGGCCCTGGGCGTGCGCTGCGGAGCCGCCTGCG |
| | | AGGACTCCACTAGGGCGCTTTCCAGGCTGGACTGCCCCGGGCTGCGCTGGAGCTGCCAGTGCTCGGGGAGTCTTCCTGGAGTCCCCAGC |
| | | TGCCCTCTCCACC |
| 92 | TBX15 | CTCTTCCCAAGTTACGCCACCGGTCGAGGACGGCAGGAGACCCCCGAGTGCAGAGAAAGCTCAAACCGGCAGCGAAGTCGGTCCTAGCC |
| | | AAGCTGAAAAAACGTCTCGGATTTCGCGGACAGCGGCCTAGACACAGCCCGATCTTCCAGTCCTAGTGCCCTGGTCGAGACGGTTCTATCC |
| | | TTTTGCAAAGAAGCCGGAAA |
| 93 | C1orf51 | TCTCGGTTGCAATCCCCACCCTCCTCACCCAGCAGGGCAGGAGGCACCCAACTTGGAGGAGAAAGGGGTGGGGGAGGTGAAACAGAGAC |
| | | CGGAGAGTCACGAGGGCTGGGCCGCCGAGAGCAGGAGAATATACCGTGTCACACACCTCCATTCTCTCACACACGTTGCAGACACAAATC |
| | | ACTGACGGTTTCCACGTGCTGCGCTCGTGAGCGGAGGTGTTCAAAGAGGGGGCAGATGAGTTACTTCCCGAGACGGAACCGGGGGTCCC |
| | | ACGTCCGCCGCCTTCAGTAGCACAACCAATCTCTGAACACTCAAACCGCGCATCTCTGGCGCATCACCATCCTATTTAAGGCCACGGGCTC |
| | | CGCCCTTTTCCTCCCCTCCCTTCTTTTCCACTCTTTTTCCA |
| 94 | chr1: | CTGCCAGAGATGTGTCTGTCTTGCGCCCCGCATGCACTGCCTGCGGGGCTGCGCTGCACTCCCCGGCGGCGCCACGGGTCTGGCCCCC |
| | 179553900- | GCGCTTCTACGTGTTGGGGGGATGCATGGACCTTGGAGATCCGTAGTTGGCCCTAACCTTCTCGGAATCTCCTCTGCACGCGCTGCCTGTT |
| | 179554600 | CCTCCTCTGCACGCTCTGTCCGTTCCTTTGCAACTTCTGTGGGAATTGTCCTGGCGTGGGAAACGCCCCCGCGCTCTTTGGCACTTAGGGT |
| | | GTGAGTGTTGCGCCCCTTGCCGCAGCGCTCAGGGCAGCATCCCGCTCGAGGATGCAGGGTTCTCACCAAGCAGTGAGGGGGACTCACGC |
| | | GCCGCCGGGGAGCGGAGCCAGGCTCCGAGAAGGGAGCAGGCTCGAGCCGCTGGGTTTTCGCAAGCCTTGGGGCCTCTGGCCGCCCTTC |
| | | CATGCCTCCGGGCGCGGGCGGCTCAGCAGGTCCCCGGCTTCGGGAAGTTTTGTGCGCGGATCGCTGGTGGGGAGGGCGCGCGGGCCA |
| | | GTGGCTGAGCTTGCAGCGAAGTTTCCGTGAAGGAAACTGCATGTGCCTTTGGAGGCGACTCGGGACTGCTGTAGGGTGGACTGGGTGTCT |
| | | ATGGAGTTGCGGGTCAGAGCGAGTAGGGTGGGTCCTTTCCTGGGACAGGACTGGGAATTGGGGCTCGAAGTAGGGG |
| 95 | ZFP36L2 | AGGGGTGTCCTCCAACATCTCTGAACCGCCTTCCCTTCCTCCTCACTGGCGCCCTCTTGCCTCAGTCGTCGGAGATGGAGAGGCGGCTGA |
| | | AGATTGGCAGGCGGCGGCCAGGGTCGAGGCTGGGAGACTCAGAGCCGCTGAGGCTGCCGGAGCTCAGGGAGCCGCTTAGGTAGCTGTC |
| | | GCGGTCCGACAGCGAGTCCGGG |
| 96 | SIX2 | TCTGACTCTCGGGCTGGAGCAGCCGAGACAGCGCTCCCCAGCGGGACTACAGAATCCCGGGTGTCGGCCTGGGGGCCCTGGATTGGCA |
| | | GTGGTGGAGTCTTCTGAGCCTAACAGCTACTAGGAATGACAGAGTTGCAGATGGCTTTGTCGCCCGCGGGGCGGCTCAAGCGTCCTGGGT |
| | | CCCAGGCCTCTGTCCTACGGCCAGGCCGCCGGCTCAACGGGCCGAAGGGAATCGGGCTGACCAGTCCTAAGGTCCCACGCTCCCCTGAC |
| | | CTCAGGGCCCAGAGCCTCGCATTACCCCGAGCAGTGCGTTGGTTACTCTCCCTGGAAAGCCGCCCCCGCCGGGGCAAGTGGGAGTTGCT |
| | | GCACTGCGGTCTTTGGAGGCCTAGGTCGCCCAGAGTAGGCGGAGCCCTGTATCCCTCCTGGAGCCGGCCTGCGGTGAGGTCGGTACCCA |
| | | GTACTTAGGGAGGGAGGACGCGCTTGGTGCTCAGGGTAGGCTGGGCCGCTGCTAGCTCTTGATTTAGTCTCATGTCCGCCTTTGTGCCGG |
| | | CCTCTCCGATTTGTGGGTCCTTCCAAGAAAGAGTCCTCTAGGGCAGCTAGGGTCGTCTCTTGGGTCTGGCGAGGCGGCAGGCCTTCTTCG |
| | | GACCTATCCCCAGAGGTGTAACGGAGACTTTCTCCACTGCAGGGCGGCCTGGGGCGGGCATCTGCCAGGCGAGGGAGCTGCCCTGCCGC |
| | | CGAGATTGTGGGGAAACGGCGTGGAAGACACCCCATCGGAGGGCACCCAATCTGCCTCTGCACTCGATTCCATCCTGCAACCCAGGAGAA |
| | | ACCATTTCCGAGTTCCAGCCGCAGAGGCACCCGCGGAGTTGCCAAAAGAGACTCCCGCGAGGTCGCTCGGAACCTTGACCCTGACACCTG |
| | | GACGCGAGGTCTTTCAGGACCAGTCTCGGCTCGGTAGCCTGGTCCCCGACCACCGCGACCAGGAGTTCCTTCTTCCCTTCCTGCTCACCA |
| | | GCCGGCCGCCGGCAGCGGCTCCAGGAAGGAGCACCAACCCGCGCTGGGGGCGGAGGTTCAGGCGGCAGGAATGGAGAGGCTGATCCT |
| | | CCTCTAGCCCCGGCGCATTCACTTAGGTGCGGGAGCCCTGAGGTTCAGCCTGACTTTCCCGACTCCGCCGGGCGCTTGGTGGGCTCCTG |
| | | GGCTTCTGGGCTCACCCTTACACCTGTGTACTAAAGGGCTGCTACCCTCCCGAGGTGTACGTCCGCCGCCTCGGCGCTCATCGGGGTGTT |
| | | TTTTCACCCTCTCGCGGTGCACGCTTTTTCTCTCACGTCAGCTCACATCTTTCAGTACACAGCCACTGGGTCTCCCTGCCCCTCCAGCCTT |
| | | TCCTAGGCAGCTTTGAGGGCCCAGACGACTGAAGTCTTACTGCTAGGATGGGAACACGATGAAAAAGGAAGGGGCCCAGTCAAAAGTCCTC |
| | | TCCTCTTCGGTTTTTCTTCAACTGTCCTTCACAAAAACATTTATTTCTGTCCCAGCGCCCTGGCGGATTTCGGCAGATGGGCCCTAGGGGG |
| | | TTGTGGAGGCCAAATTCCCAGGATGCTGGTCCTGCCTTTTTCATTGGCCAAAACTGTATTTCCTACAACGACTAAAGATAACCAAGAACTG |
| | | AGTAGACCCTGTTCTCTCACCAGATCTCCCTGGCTCTGTTTAACTTTTCCTGGTGCAATGCGATGGCACCACCAGCTCCCCAGGCAGGCAC |
| | | CACTCCCTCAAGATACCATTTGGGGTAGGGATTTGAGTCCTGGAGAGGGTCAGCGGGGCGCCGGGGTGGGGGTGGGAAGGAGACTGACAG |
| | | GGACACACCGCGAGCTCCGCATACTCTCCTCTGCCCCCTGTAGCCCGGGGCTTTAATGACCCCAAGCAGATTTCCTGTCTCTGGTCTAGCC |
| | | AGCTGCCCCTAGGGCTGGATTTTATTTCTTCATGGGGTTTCACCCTAAAGGGCCCCCTGGTCATGGGACCTGGTTGGGAACAAATGAAAGA |
| | | TGTCTTGTAGCAAATGCTTTCAGGGGAGCAGAAAAGAAGATTGGGCACTTCCAGTCACTTGGTCACTTTAGGTGGCTGGAACAAAACTGGT |
| | | GACTTTCACGACTGCTACAGGGTGAGGGGGTGAAGGGTGGCAGAGAGGTGACAAGCCACTGGGAATCCTATTCAGTGGGGATGCCGACAGG |
| | | GAGTGGCTGTAATCAACTGAGCAACATCTGTGTGAATGTTATTCACAGGTCAGGACAGCAGCTTGGTCTTCCCAGGTGAGGAACTGAGGAC |
| | | TGGCCTGCATAGATTTGTGCAGTAGGTGAGTAGCTTCCAAATTTATTTTCAGAACTTCCATGTAGTACCTGCCTCTCCATTTAAATATTTT |
| | | TTAAAATTTTATTTATTTAAATATTTTCTTGGTTAGCTTTCCAAGAGGGAGGAAAAGAGGGGAGTTGCAACAAGTAGTGCCCCTATGCTGG |
| | | GATTCATTTTCCAGAGTAAAGCCTGGGACTGGCACCCTGACCCCTACCGGCAGGTGAAAACTCCAGGCAAACTGCTGAGATCCCACCTGGG |
| | | CTGGCTGAGATAGTGCCTGGGGTGCATCCCTCAGCAGCTGCCACCTGGGCCCTGGGGCCATCTCTTTCTCTGGCATCAAGCAGCCAGGTGT |
| | | CAAGGCCTTCCCAGCAATCCATGCTGCATGGCTGGGTCTTGTTCTAGCAGGTCGATGGGCAGGGACTGGTAGCTTAGCCAGGGCACCAGTG |
| | | CGTGGCTGTGGGTTTGTGTGCTTCTGTGGAGAAGCATGATGTGTATGTGTGTGTGTGGGCACAGGCATGAGGAAGGGTTCATTTGTGCAG |
| | | GTATCTCCCATGTATATCAGTGTGGGAGAGTGCCTGAGGATGTGTTTGTGTGTCTGAAAATGGGCGGAGGGTCTGTTGTGCTAATGTGTGC |
| | | AGGGGTGAACATGTGTGTGACAGTCTGTGTGTTTCCCTGAGTGGTGGCTGCGTGAGAGGGTGAGGGGATTTGGTGTTGTCTACCATGCCCG |
| | | GCACATAGCAGGCTCTTAATAATCTTGAATTTAATTAATGTTAAATGTGTATGTTCCCATCCTTGTGGAAGTTGGTATAGAGCCTGTTTTC |
| | | CTGTGATTGTGAGACTGGAAAATGGGGGACGGGCAGGGGCGAGACAGGATACAGAGGCTACTGTTTTCTTCCTCCCTAGAAGTAAGTACAT |
| | | AGAAGAGTGGGCTCTGGCACCTCACGGGACATCACCAAGTCCTGTGTGGCTGGCTAGGCTGTCCCAAGGTGGCTTCAGGCATCACTTGAAT |
| | | CTTTTGAGACCTTCAGGCAGTAGCCTGCCATTCACCCTGTCAGTCAGCAGAAGTTGGGCCCACACAGGCCATAGAAACACAGAGCAGTTCC |
| | | CGGGAGGACCTGAGCTGTCCCTGAGAGCAGAGCTTCCAGGAGAGGCCGCAGGAACTGCCTTGACCGGAATTCCTCTTGGGGTGCAAAGG |
| | | TGGAGGGACACATGGTGCGACCCCAGGCAGAGGACTGCAGCCACTCCGTGCAGTCCCAGCCTCTGGGGTAGCCCCTTGACCTCCAGGCC |
| | | TGCACAGATCCAAGGCCGAGGTCCAGGCTCCAGCGCCAAATTAGCTGGCCTAGCAGCCTGCAGCCGCTCTAATCTCAACTAGGAAGGAAT |
| | | CCTTGCGCTTAGAAAGTCCAAGCGAAAGGGTATTCTGATTTTATCCCGGTTTTACCAGAAAATGCTGAAAGGAAAAGCCCCGAGAGGACAC |
| | | AGTGCTCTAGGAACTCGGGGCGCCACGAGCGCCTCATCCCCTCCCTTCCGCCCGGCCGCGGTGCCCTGGTCGCTGAGGGACGCGGTCA |
| | | GTACCTACCGCCACTGCGACCCGAGAAGGGAAAGCCTCAACTTCTTCCTCTCGGAGTCCTGCCCACTACGGATCTGCCTGGACTGGTTCA |
| | | GATGCGTCGTTTAAAGGGGGGGGCTGGCACTCCAGAGAGGAGGGGGCGCTGCAGGTTAATTGATAGCCACGGAAGCACCTAGGCGCCCC |
| | | ATGCGCGGAGCCGGAGCCGCCAGCTCAGTCTGACCCCTGTCTTTTCTCTCCTCTTCCCTCTCCCACCCCTCACTCCGGGAAAGCGAGGGC |
| | | CGAGGTAGGGGCAGATAGATCACCAGACAGGCGGAGAAGGACAGGAGTACAGATGGAGGGACCAGGACACAGAATGCAAAAGACTGGCA |
| | | GGTGAGAAGAAGGGAGAAACAGAGGGAGAGAGAAAGGGAGAAACAGAGCAGAGGCGGCCGCCGGCCCGGCCGCCCTGAGTCCGATTTC |
| | | CCTCCTTCCCTGACCCTTCAGTTTCACTGCAAATCCACAGAAGCAGGTTTGCGAGCTCGAATACCTTTGCTCCACTGCCACACGCAGCACC |
| | | GGGACTGGGCGTCTGGAGCTTAAGTCTGGGGGTCTGAGCCTGGGACCGGCAAATCCGCGCAGCGCATCGCGCCCAGTCTCGGAGACTGC |
| | | AACCACCGCCAAGGAGTACGCGCGGCAGGAAACTTCTGCGGCCCAATTTCTTCCCCAGCTTTGGCATCTCCGAAGGCACGTACCCGCCCT |
| | | CGGCACAAGCTCTCTCGTCTTCCACTTCGACCTCGAGGTGGAGAAAGAGGCTGGCAAGGGCTGTGCGCGTCGCTGGTGTGGGGAGGGCA |
| | | GCAGGCTGCCCCTCCCCGCTTCTGCAGCGAGTTTTCCCAGCCAGGAAAAGGGAGGGAGCTGTTTCAGGAATTTCAGTGCCTTCACCTAGC |
| | | GACTGACACAAGTCGTGTGTATAGGAAGGCGTCTGGCTGTTTCGGGACTCACCAGAGAGCATCGCCAACCAGAACGGCCCACCCGGGGT |
| | | GTCGAGTCTTGGTAGGGAAATCAGACACAGCTGCACTCCCGGCCCGCGGGCCTTGTGGCATATAACCATTTATATATTTATGATTTCTAAT |
| | | TTTATTATAAAATAAAAGCAGAAATATTTCCCGAAGAACATTCACATGAGGGCATTACGGGGAGACGGCAAGTCGGCGGCTCGGGGGGCGC |
| | | GCTCAGCCGGGAGCGCTGTAGTCACAGTCCCGGGAGGAAGAGCGCG |
| 97 | chr2: | TGGAACAAGTGTCAGAGAGTAAGCAAACGACTTTCTGAGCTGTGACTCTGCTCCTCGACTGCCCACGTGCTCTCCGCTGTCTGCACTCCTG |
| | 137238500- | CCTCACCTGGGCTGACTCGGACTCTCCACCTCCTTTGCTGCTTCCGGCATGAGCTACCCAGGAGCCTAAGGCGCTCCTTCCCGCAACTCC |
| | 137240000 | GGTCCCCGCGCCCCGGGACTGCAAATCCTTTAAACAGAGGCCCCAGAGCTAGGGGTTTTCCCAGGCTCTGGTGGGCGTGGGCTGACAGT |
| | | CGCTGGGAGCCCCGCAACAGGGGGGATGTCCAGGCAGGTATGCACCCAGCTCCCGGCGTTTCCCGGAGTCACCACAATGTTTCCCTTTCT |
| | | CTCTCCCCCACGTATGCTGCTAGGGGTACTCCCCAGATAGGATTTTCTTTGTCTTTTCTCCTAGTAACACCGAAGCCCTCTCGTGCCCGGG |
| | | GACTGCAGAGGAACGCCAGACCATCCGGACCTTGCGGGATGGCTCGGTGTGTGTGTTTTACTGTGTGTCGGAGTGTCGCGCATGTGTGCG |
| | | TGTTGGGGCGCGTTATCAACAGGGGCCTAGGGCACCCCCACTCTTTCTTGCTCTCTTCCCCCATCACTTCATGGACCTCCGAGGCGCAAAG |
| | | CGCTCGACCCTCTCCTGGGCTCAGTGGCTTGGGTACTCCGGGCTGAGCTCAGCTGGGGAGTCCCCTTACCCAGCCCGCACCGGCACCCC |
| | | GAAGCTTCAAAGTTGCGGCAAACAGTTGCGGGGAGCAGAGGAACTGAGGTCCAGGCCAGCGCGCCCGCGGTCGCTCGCCTTGGGGAGC |
| | | AGGCTGAGCCGAGGGTCGTGCGGGTGCGCGGCAGAGGCGGTAGGAGGCGGAGGAGAGGGGGGAGAAAGAGGGGGCGGTGGGGAACA |
| | | GCTGCCGGGGTAGGCGAGGCGCAAGGTGGCTCCCCGCGGCCCCGCGCCCCGCGGCTCTCGGACGCACCAGGCAGCCAATGGCTGCGC |
| | | AGAGGTGTACAGCAGATGGCGTCTGACTGCGCCGTTCCTTCCTCCTCCTCCTCCTCCTCCTTCTCTTCCTCCTCCTCCTTCTCTTCCTCCT |
| | | CCTCCTCCTTCAGTGCTGAGGAGCCAGAGTCGCCGCCGGGTTGCCAGACGCTGGAATGGGTGGTCTTCCGACACACACCACCATCTTTCTT |
| | | GCGCTCGGGAAGCTCGGGGCTCAGCGGCTCCCAGAGGTTACGGCGGCGGCTCTGGCGAGACGGGTGAGTGCAAGCACGCGGAGCCCC |
| | | GAGTCGGGGATGCCGGGCCCCCTGGCCGGCCGACTGGGGCGCGGGGTGGCAGCGCCGGGGAAGGGGGCGCGCTGCCGGCGCAGACT |
| | | TTGCTCTTTCCTCGCCGGACAGCCATCGTCGCCCCTTCTCCCAGCCAGACGCGGGAACTTGGAAGCGGATCTTCTCGGACGCCTCTGGCT |
| | | TGGGGCTGCGGGAAGCGTGGGCTGCCCGGGGCGCAGTGTGCGGAGACCCTCTAGGCGGGCGGGGACGCCCCAC |
| 98 | MAP1D | GTTATTATCCACGGGGTCCTAATTAAAGCTTGATTAAAATGCCCTTCTTTCTCTAAAAAATTACGAACTAGGCAACTTCATACATTTTGAA |
| | | TGGCGCAGTGTTTCCTCTTCCAACTGTTTAGTTTGTAGTATACTATGTAAGCAACATCAATTATCAACCCTTGCAAGATGACAACATGAGC |
| | | CTGTGGGGGAAGCACTTGAGGGGAGGGAGGAGAAACTTCTCTTTTTTAATAATCAGCCGGAAACAATGTTTAACAAGAATCTGATGAGGTC |
| | | ACTGCAGTAAATATTTTTCCTCTTACAGAGCCAATCATCACGGAGGGATCCCCTGAATTTAAAGTCCTGGAGGATGCATGGACTGTGGTCT |
| | | CCCTAGACAATCAAAGGTGTTTGCTTTCTGCTCTGTTGCTTTTAAATTGTATGGGAAAGGAAGATTGGTCCGACGGCGCGCTTGTGGCCCG |
| | | GCCGGAGCTTGCGTGCGCGTTCTGACGGCTGGGTGCTGTGTTACAGGTCGGCGCAGTTCGAGCACACGGTTCTGATCACGTCGAGGGGCGC |
| | | GCAGATCCTGACCAAACTACCCCATGAGGCCTGAGGAGCCGCCCGAAGGTCGCGGTGACCTGGTGCCTTTTTAAATAAATTGCTGAAATTT |
| | | GGCTGGAGAACTTTTAGAAGAAACAGGGAAATGACCGGTGGTGCGGTAACCTGCGTGGCTCCTGATAGCGTTTGGAAGAACGCGGGGGAGA |
| | | CTGAAGAGCAACTGGGAACTCGGATCTGAAGCCCTGCTGGGGTCGCGCGGCTTTGGAAAAACAAATCCTGGC |
| 99 | WNT6 | TCCCTGCTGTGGGACCCGAGGAGAGGAGAACTGGTTCGCT |
| 100 | INPP5D | TCTCTCTCTCTCTCTTGCTTGGTTTCTGTAATGAGGAAGTTCTCCGCAGCTCAGTTTCCTTTCCCTCACTGAGCGCCTGAAACAGGAAGT |
| | | CAGTCAGTTAAGCTGGTGGCAGCAGCCGAGGCCACCAAGAGGCAACGGGCGGCAGGTTGCAGTGGAGGGGCCTCCGCTCCCCTCGGTGGT |
| | | GTGTGGGTCCTGGGGGTGCCTGCCGGCCCGGCCGAGGAGGCCCACGCCCACCATGGTCCCCTGCTGGAACCATGGCAACATCACCCGC |
| | | TCCAAGGCGGAGGAGCTGCTTTCCAGGACAGGCAAGGACGGGAGCTTCCTCGTGCGTGCCAGCGAGTCCATCTCCCGGGCATACGCGCT |
| | | CTGCGTGCTGTGAGTACAACCTGCTCCCTCCCCGGGCACAGATATGACAGAGGGGCTTAGAGGGGGCCCAGCTTTGAGATGGGTTGTTCT |
| | | TATGTCACAGGACAGAGTGATCTGACATGCACACTTCCCCGCCACCCTGTCAT |
| 101 | chr2: | TGTCCTCGAAGAAGGGCCTGAGCAGCAGCAGAGGACCCCAGGCGACCGTGCCTGAGCCGGGCGCCGACGACGACTGAGCACCTGATAT |
| | 241211100- | GTCCCCGGCACTCGCAGCCCCGCGGCCGGAGTCGCTGTGGGTGAGCGGTCGTCGAGCTTCACAGAGGCCGGGCTCTGTGCCAGGGCCC |
| | 241211600 | CGACAGGGCAGGAAGCAGATAGAGTCCCACAAGCACAAGCCCAGTGCGCAGAAAGGGTTACTTAAAAAATAAGTTCTGTGATAAAATCAA |
| | | ACAGGGTGAAGGGCTGGAAACAGGTCATGAGGGCGCAAACAGGTCGTGAGGGCGCAAACAGGTCGTGAGGGCGCAAACAGGTCGTGAGG |
| | | GCGCAAACAGGTCGTGAGGGCGCAAACAGGTCGTGAGGGCGCAAACAGATCGTGAGGGCGCAAACAGGTCGTGAGGGCGCAAACAGGT |
| | | CGTGAGGGTGCAAACAGGTCGTGAGGGCGCAAACAGGTCGTGAGGGTGCAAACAGGT |
| 102 | WNT5A | AAATGAGACCTCTGGGGAGACTGTCAACCCCAGGGGTAAAACAAAAATTCTGATCAGAAACTGAGTTTCCCAAAGAAGGGGCTAAATGTTT |
| | | TCCAACACTTTCGGGGCTCAGGGAAGATGACTCTGTAAGGACACTGAGAATCTTCCTCGCGTGCCACGGGGAGGAGGACTGGGGGCGTTTG |
| | | AGGGGCTCAGCGCACCAGAGGAGTGAGGTGGAGGAGGGCGTTCCCGCGTCCTCCTCTTCAATCCAGAGCAGCTCAACGACGTGGCTCCC |
| | | TTTCTATGTATCCCTCAAAGCCTTCGCGT |
| 103 | chr3: | TAGGCTCTAGTGGACCTAGCAGTGGGAGAGCTACTTGGGCTGGTTTCTTTCCTGACGCTGCAGGGATGGGCATCGGCCTGGAACCAGAAG |
| | 138971600- | CGCAGGAGCTGGGCCACGGCAGAGTAATTAAGAAAATAATGAAATTGATGGCGGATGGGGGCGCTAGAAATCCTGGGGCGTCTACTTAAA |
| | 138972200 | ACCAGAGATTCGCGGTCGGCCCCACGGAATCCCGGCTCTGTGTGCGCCCAGGTTCCGGGGCTTGGGCGTTGCCGGTTCTCACACTAGGA |
| | | AGGAGCCTGAAGTCAGAAAAGATGGGGCCTCGTTACTCACTTTCTAGCCCAGCCCCTGGCCCTGGGTCCCGCAGAGCCGTCATCGCAGGC |
| | | TCCTGCCCAGCCTCTGGGGTCGGGTGAGCAAGGTGTTCTCTTCGGAAGCGGGAAGGGCTGCGGGTCGGGGACGTCCCTTGGCTGCCACC |
| | | CCTGATTCTGCATCCTTTTCGCTCGAATCCCTGCGCTAGGCATCCTCCCCGATCCCCCAAAAGCCCAAGCACTGGGTCTGGGTTGAGGAA |
| | | GGGAACGGGTGCCCAGGCCGGACAGAGGCTGAAAGGAGGCCTCAAGGTTCCTCTTTGCTACA |
| 104 | ZIC4 | GAGGTTGCTGACTCAGGAGCCAGGAGCTGAGAAACTCCTAGGCTAGCAGCCGTTGAGCCTAATTTTATTTTCTGGCTTTCTCCGAAATGTC |
| | | TCGTTTCCCTCATCTTTCTGGTCCTTTTCGTCTCTCTTATTTTCCCCAAAACGTCTACCTCACTTCGTCTTCCTTTCTCCTCCCCTCCCCC |
| | | TCTCTTTCCTCTATACTCTCTTCCCATTTAGCCTTGCAGGCCCCTCCTCCCCGGTGTTGGAGAGCTCAAAGACGCGCGAAACTCAAGGATC |
| | | TGGCCCTGACCAGGGACGGGATTAGGCGGGAAGTGGTGACGGCCTGAAAAGGCTGGGCTCGAACCCGTGCCTTCCTGAAAGGACTCTCCCC |
| | | GCCACAAGTCACACCCACCCGCAGGCCTGCTGGCCAAAGAAACAAAGGAGTCGGGCGTGGATCCAGGAGAAACAGGTTTTCGCTCTCGGAT |
| | | CTCCCTGGGCAAATCAGGGATCCTGAGCGCTATACCCCGCAGTCGTACGGAGCCTCTGGGAAAGGGGATTTAAGGGTGACTTCCACTTTCA |
| | | GCTTCGGCTACTTGTTGCCTGCGGTCCAAGCCTTCTCTGCTTCCTCCTACCTCGTCTTAGGCCTCTGTAGAAAGTGCACGCCGCGTTTCCC |
| | | CTTCCAGGCTCTGAGAGGGCCTGCAGGCCCGTGGCCGCCTCCGACAAGATGCCTTCCAGTGCTAGGGGGGCCACTTTGGCGGGATGGGG |
| | | GTCGGTTGGTTAAAAAAAACTTAAGTTCTGGCTCAGTCGAGTGTGGCAAAAGCCGAGGGTCGGGGGTTGGGGGG |
| 105 | FGF12 | TACTGACCTGGTCTCCGCCTCACCGGCCTCTTGCGGCCGCTGCAGAAGCGCACTTTGCTGAACACCCCGAGGACGTGCCTCTCGCACAGG |
| | | GAGCGCCCGTCTTTGCTGGGGCTGGAGCGGCGCTTGGAGGCCGACACTCGGTCGCTGTTGGACTCCCTCGCCTGCCGCTTCTGCCGGAT |
| | | CAAGGAGCTGGCTATCGCCGCAGCCATAGCTGCTCAGCGAGGGCCTCAGGCCCCAGCCTCTACTGCGCCCTCCGGCTTGCGCTCCGCCG |
| | | GGGCGAGGGCAGGACCTGGGCGGCCAGGGAAAGGGCAGTCGCGGGGAGGCAGTGCTAAAATTTGAGGAGGCTGCAGTATCGAAAACCC |
| | | GGCGCTCACAAGGTTAGTCAAAGTCTGGGCAGTGGCGACAAAATGTGTGAAAATCCAGATGTAAACTTCCCCAACCTCTGGCGGCCGGGG |
| | | GGCGGGGCGGGGCGGTCCCAGGCCCTCTTGCGAAGTAGACGTTTGCACCCCAAACTTGCACCCCAAGGCGATCGGCGTCCAAGGGGCA |
| | | GTGGGGAGTTTAGTCACACTGCGTTCGGGGTACCAAGTGGAAGGGGAAGAACGATGCCCAAAATAACAAGACGTGCCTCTGTTGGAGAGG |
| | | CGCAAGCGTTGTAAGGTGTCCAAAGTATACCTACACATACATACATAGAAAACCCGTTTACAAAGCAGAGTCTGGACCCAGGCGGGTAGCG |
| | | CGCCCCCGGTAGAAAATACTAAAAAGTGAATAAAACGTTCCTTTAGAAAACAAGCCACCAACCGCACGAGAGAAGGAGAGGAAGGCAGCAA |
| | | TTTAACTCCCTGCGGCCCGCGGTTCTGAAGATTAGGAGGTCCGTCCCAGCAGGGTGAGGTCTACAGAATGCATCGCGCCGGCTGCGGCTT |
| | | TCCAGGGGCCGGCCACCCGAGTTCTGGAATTCCGAGAGGCGCGAAGTGGGAGCGGTTACCCGGAGTCTGGGTAGGGGCGCGGGGCGG |
| | | GGGCAGCTGTTTCCAGCTGCGGTGAGAGCAACTCCCGGCCAGCAGCACTGCAAAGAGAGCGGGAGGCGAGGGAGGGGGGAGGGCGCG |
| | | AGGGAGGGAGGGAGATCCTCGAGGGCCAAGCACCCCTCGGGGAGAAACCAGCGAGAGGCGATCTGCGGGGTCCCAAGAGTGGGCGCTCTTT |
| | | CTCTTTCCGCTTGCTTTCCGGCACGAGACGGGCACAGTTGGTGATTATTTAGGGAATCCTAAATCTGGAATGACTCAGTAGTTTAAATAA |
| | | GCCCCCTCAAAAGGCAGCGATGCCGAAGGTGTCCTCTCCAGCTCGGCGCCCACACGCCTTTAACTGGAGCTCCCCGCCATGGTCCACCC |
| | | GGGGCCGCCGCACCGAGCTGGTCTCCGCACAGGCTCAGAGGGAGCGAGGGAAGGGAGGGAAGGAAGGGGCGCCCTGGCGGGCTCGGG |
| | | ATCAGGTCATCGCCGCGCTGCTGCCCGTGCCCCCTAGGCTCGCGCGCCCCGGCAGTCAGCAGCTCACAGGCAGCAGATCAGATGGGGAT |
| | | TACCCGCCGGACGCAAGGCCGATCACTCAGTCCCGCGCCGCCCATCCCGGCCGAGGAAGGAAGTGACCCGCGCGCTGCGAATACCCGC |
| | | GCGTCCGCTCGGGTGGGGCGGGGGCTGGCTGCAGGCGATGTTGGCTCGCGGCGGCTGAGGCTCCTGGCCGGAGCTGCCCACCATGGT |
| | | CTGGCGCCAGGGGCGCAGGCGGGGCCCCTAGGCCTCCTGGGGCTACCTCGCGAGGCAGCCGAGGGCGCAACCCGGGCGCTTGGGGCC |
| | | GGAGGCGGAATCAGGGGCCGGGGCCAGGAGGCAGGTGCAGGCGGCTGCCAACTCGCCCAACTTGCTGCGCGGGTGGCCGCTCAGAGC |
| | | CGCGGGCTTGCGGGGCGCCCCCCGCCGCCGCGCCGCCGCCTCCCCAGGCCCGGGAGGGGGCGCTCAGGGTGGAGTCCCATTCATGGG |
| | | CTGAGGCTCTGGGCGCGCGGAGCCGCCGCCGCCCCTCCGGCTGGCTCA |
| 106 | GP5 | GGGGGACACAGAGAGGAGGGGTTGCGGGCCTGTGAGAATGAAGAGCACAGAGCGGAGAGGGGGAGGAGGAGGGAAAGGAAGGCGTGG |
| | | CAGTGAGAGAGAAGAGGAAGAAGAGAGGAGGAGTGGGGAGGGGAGGGAGAGCAAGACAGCAGCGGGTCTGGATTCCCCTCCGAGCCACATC |
| | | TGGTCAGGTTCTAAGTAATTAGAAGATTTTCCCATTGGTTTACCCAAGGGCTCTCTCTCTGATTAATTTTCGAAAGAGTTGGCCAATTTTA |
| | | ATCATAGCAAACACGATGATCACGGTGATCATGGCCTGAACAGCTAAAAGCAGAAAATAAAACCCCCAGAACGGACTATGATCTTGACCTT |
| | | TGCCCGTGGTCACCGGCTGGGCCCACACCCAGGGTTCTGAGCTGTTGGGAGCCAAGGCTGGGTGGACAGGGGCTTCCGAGGAGCTGTCC |
| | | GCAGCGGGGCGGGGAGGCGGGCCCCGGGGGCCCGGGCACTCCGCGTCACCCCCCGGCAGGGCCCAGAGCGGCAGGCCGGCGTGCGC |
| | | CCCAGGGCCTGCGCACCGTGGGGGCTCTTCCCCGCCCACGAGGCCTAGGTGCTGCCGCAGCCACCCCAGGAAGGGCCCCAGGCCACAG |
| | | TCGCAGCGCCAGGAGTTGTGCCCCAACAGGACCTCCGTCAGCCGGGGCAGAGCCCCAAACACGTCGCCAGGCAGGGTCTCCAGCTGGTT |
| | | GTGGTCGAGCTGGACGCTCTCCAGGCTGCTGAGATTGCGGAAGAGGGCACGGGGCAGGGCGCGCAGCCTGTTGCGGCGCAGGGACACC |
| 107 | MSX1 | GCCCCGGTGCACCGCGCGTCCAGCCGGCCCAACTCGAGCTAGAAGCCCCAACCACTGCCCAGTGCCTGAGTTGCAGTCTTGGGTCCTTTA |
| | | GAAACCTGGAGATGTGCGTAAAATTCAGATGCCGGTATTCCCGAACTTCCCCAGGCCTCAGCATATCTCGGCGGCCTGTGGACAGATGGG |
| | | AGGCTACCAATCGCTCCGGCGTCCGCAGCCCGACCCCTGCCGCCAGACCCCGGACGTCTTCCGGATAATAAAGTTCCCGCTCTAATTCAT |
| | | TTTCCCTAATCTGGACGCCCCTAATCTACAGCTTTTATTGCGCCCAGTTAAAAGTCGAGGGAATTCGCTGTCCCTCCGCGCTCGGATAATT |
| | | ACCCCTAAATGGCCACGGCAGCCCCTTGTGTTTCCTGGAGATTAGAACCCCGCAGTCATCAATGGCAGGGCCGAGTGAGCCGCCAATCACC |
| | | TCCGCTCACTCCCTGAGAGCCGCTGGCCTGGGCCGCAGGAGGAGAGGCCATAAAGCGACAGGCGCAGAAAATGGCCAAGCCCCGACCC |
| | | CGCTTCAGGC |
| 108 | NKX3-2 | AGGGTGCCTCTGTTCAAATTAGAAAAAGGCGCCCCCTCAGGGCAGACTCAGCCCAGCTGCCAGGGGACAAGTCCTGGCTAACGGGAGCT |
| | | GGAGCTGGGTTTCACCTCCAGGTGCCTCCTTGGCGGGGCGCCCCGTGCAGGCTACAGCCTACAGCTGTCAGCGCCGGTCCGGAGCCGG |
| | | AGCGCGGGAATCACTCGCTGCCTCAGCCCAAGCGGGTTCACTGGGTGCCTGCGGCAGCTGCGCAGGTGGAGAGCGCCCAGCCTGGGAG |
| | | GCAGTAGTACGGGTAATAGTAGGAGGGCTGCAGTGGCAGAAGCGAGGGTGGCCGCAGCACTTCGCCGGGCAGGTATTGTCTCTGGTCGT |
| | | CGCGCACCAGCACCTTTACGGCCACCTTCTTGGCGGCGGGCGCCGAGGCCAGCAGGTCGGCTGCCATCTGCCGGCGCTTTGTCTTGTAG |
| | | CGACGGTTCTGGAACCAGATTTTCACCTGCGTCTCGGTGAGCTTCAGCGACGCGGCCAGGTCTGCGCGCTCGGGCCCGGACAGGTAGCG |
| | | CTGGTGGTTAAAGCGGCGCTCCAGCTCGAAGACCTGCGCGTGGGAGAAAGCGGCCCGCGAGCGCTTCTTGCGTGGCTTGGGCGCCGCC |
| | | GGCTCCTCCTCCTCCTCCGCGACGCCTGCCGGCCCGCTGCCGCCCCCGCCGCCGGCCCCGCTGCACAGCGCGGACACGTGTGCACCTC |
| | | TGGGGCCAACACCGTCGTCCTCGGTCCTTGGGCTGCGGTCGCCTGCGGACCCCGGTGGGAACAGAAACAAGAGACTGTCAGCGCCACAG |
| | | ACGAGGTGAGGCCGGGCCTCAACTGCAGGGGTCACGGGAGTGGGGCGGAAATACACTTTGATCCCACTCAAGCGGAGCGGAGGTCTGG |
| | | GAGGCCCTGGGCCCGGGAGACCAGTCTTAGACTCTTGCCCCACTGGGTATCCCATCTAGGCCTCTTCTGGGGAGGGCGGCAGACTCAGC |
| | | CGCTGTGTCAACGCTGTGTTGTCGAGACCAGCTCCCCACCCTCTCTGGGCCCCAGGCTCCCCTCAGTAACTTGGGGCACTCGACCCGAGC |
| | | ATCCGCGAAAGCCCTCCCGGCTCTCAGCGTTGAGCATTGGGATTCTAGACTGCATTTCCGTCTCTCTGCTTGGGTTCACGCGCCTCTCCA |
| | | CACTTAGTTCACACGCACACACGCGCGCGTCCTCGCAGCACACACTTGTCTGGTGCAGGTAAGGGAAGGTGGAGGCGGATCCTGGGGCCA |
| | | AAGGTATTTAGAATCTTTCACCCTCAGCCGCCTGGGATTGCTGTGAGAGACATGGAAACAGGCTGAGCCGAGGCCTTAGATGAGAGGATG |
| | | GACTGGAGAGTAAAGAGGGAGGGTTGCCCCTGCATCGAGTTTTTGGACCCTGATCCCACACCAGCTTCTCGGTCTCGTACCCGCCCTTCC |
| | | GAAGAACTCCAGCAGAAAGGTCCAGCGGTCCCCTGTGCTTGAGGCCTACAGAAGCTTGTACCCAACTAGGGCAGGCACCCGGGTCTTCCA |
| | | GACCACAGGACAGGACAGGCCACGGCTGAGGAGGCCTCTCTCCTGCCTCCAGGATGAACTAAAGACCCAATCCGGGATCTTCGGCCTAG |
| | | GGCTGCTCTCCCAGACCTGGGGTCTGAGAAAGCCAAACCAGCCCTTTCCCCAAAGCTCTAGTTCTGCAGATTCTCAGCTCTGGCCCACTCG |
| | | GAGGTGTTCTTCACCACCTATCCACCTACTGTGGGGCCCGGCCCTGGGACCTTGAACTGGCAGGTCTCTGGTCCAGAGCTAGGTCACTGG |
| | | CTACCTGAGGTCTCTGAACCCCTCACTTTTCCGCTTCCCTGATTTTGGGGATTTGGGGACAGACACGGCAGAAAGCACTGGCGACGAACTC |
| | | AAAAACTCCCGAACGCAAGGGGCAGCGGTTCTCCCAACCCAGTCTAATGCACATTGGCCCAGGATGTCTCAGGCCTCACCCCAGGACGTA |
| | | GGGCTCTGAGGAGCTACTCCGGTCTCTCGCGGGCT |
| 109 | chr4: | GAGAAGGGATGTGGCGGGGGGCTCCTCCGGCCCTGGACTCCCTGGGTGGACTAGAAAAGGGCAAAGAAGTGGTCACATCTGTGGGCCAG |
| | 111752000- | ACTGGTGCGCGATCTTTGGAGGCGCAGCAGCAAGGCCGCGCCAGGGCTGAGCCCAGACCGCCCACGAGGAGGCCCGCCAGGCCCGGA |
| | 111753000 | GCAGCGGCGCGTGCGGGGGCGTGCCGAGCGCAGGCTCTAGGGCCCCTGCTTCGCCCCAGCTGGACCCCGCGGGCGGTCGGTGCAGCT |
| | | CGAGCGTGTGGGCTGCGATGCCCTGCCTGAGACTTCGGGCTAGGGATGCGGGCGGGAAGTGGGGGTGCGGCGGCAGCTGCAGATTAGA |
| | | TTCCTTTTTTTTTTGGCCGGAGGGACGTGCAAACTTCTAGTGCCCGGGCCAAGAGGGCGACCCCGGAGGTGCGTAGGTGGCCCTCCGGGT |
| | | TCCCGCTTCTCCTAGTGCCTCTGAAAATACCGTCAGGGTAAAGGGAGACAGGCAGTAAGTCTTACCACCACCGCCCTTTCCCCATGTCATT |
| | | GGCCAAAAACTGAACATTAAGATAAAGCAGCTGTTTCAGTCAATGGAAAGCGGTAGGGCGAGGTTGTACCCAAAACCCGGTTTAGACGGCC |
| | | AATGAAGTCCTAGGAAAAGCCGCCCCGGGGGCACGTTCAGGTGGAGCGGCTGCACCTCGGGTCGTTCTAAGGGATGGGCTGCGTGGTAC |
| | | CCACGGAATTCATGGGTCCAAAAGGTCCTGGTCACCTGTCCAAACATCCATCCCCTGGCGCATGGCGGTTGACAAGATGGCCCGGCCACC |
| | | CAGAGGAAGGAGGATCCGGGACGGGGAACTTCGCGCCGGGAAGCTGTAGCCCAGAGCTGCAGCTCAGCATTCGCAAGAGATTCATCTTTT |
| | | TTTTCTCTCGTGTTCGGAGAAACAGATAAACAAGACACCGCCTCATCAGATAAGAACGTCTCCTTCGATGTCACGGATTTCAAGAGGTAGC |
| | | TGGAGAAACTGACGTCA |
| 110 | SFRP2 | CAGGTCAGGCAGAACTTCTGCCCTTCCCGCTACTGGCACCCCAAGCAGGGATGCACTGGGATGCGTGGCAGGGGCGGGATCTCCTGGGA |
| | | GCGTCTCAGCCCAGCAGGGAGTGGGGAAGCAAGAGGGAAGGCTTACCTTCCTCGGTGGCTGGCAGGAGGTGGTCGCTGCTAGCGAGGG |
| | | GGATGCAAAGGTCGTTGTCCTGGGGGAAACGGTCGCACTCAAGCATGTCGGGCCAGGGGAAGCCGAAGGCGGACATGACCGGGGCGCA |
| | | GCGGTCCTTCACCTGCACGCAGAGCGAGTGGCATGGCTGGATGGTCTCGTCTAGGTCATCGAGGCAGACGGGGGCGAAGAGCGAGCACA |
| | | GGAACTTCTTGGTGTCCGGGTGGCACTGCTTCATGACCAGCGGGATCCAAGCGCCGGCCTGCTCCAGCACCTCCTTCATGGTCTCGTGGC |
| | | CCAGCAGGTTGGGCAGCCGCATGTTCTGGTATTCGATGCCGTGGCACAGCTGCAGGTTGGCAGGGATGGGCTTGCAATTGCTGCGCTTGT |
| | | AGGAGAAGTCGGGCTGGCCAAAGAGGAAGAGCCCGCGCGCCGAGCCCAGGCAGCAGTGCGAGGCGAGGAAGAGCAGCAGCAGCGAGC |
| | | CAGGGCCCTGCAGCATCGTGGGCGCGCGACCCCGAGGGGGCAGAGGGAGCGGAGCCGGGGAAGGGCGAGGCGGCCGGAGTTCGAGC |
| | | TTGTCCCGGGCCCGCTCTCTTCGCTGGGTGCGACTCGGGGCCCCGAAAAGCTGGCAGCCGGCGGCTGGGGCGCGGAGAAGCGGGACAC |
| | | CGGGAGGACAGCGCGGGCGAGGCGCTGCAAGCCCGCGCGCAGCTCCGGGGGGCTCCGACCCGGGGGAGCAGAATGAGCCGTTGCTGG |
| | | GGCACAGCCAGAGTTTTCTTGGCCTTTTTTATGCAAATCTGGAGGGTGGGGGGAGCAAGGGAGGAGCCAATGAAGGGTAATCCGAGGAGG |
| | | GCTGGTCACTACTTTCTGGGTCTGGTTTTGCGTTGAGAATGCCCCTCACGCGCTTGCTGGAAGGGAATTCTGGCTGCGCCCCCTCCCCTAG |
| | | ATGCCGCCGCTCGCCCGCCCTAGGATTTCTTTAAACAACAAACAGAGAAGCCTGGCCGCTGCGCCCCCACAGTGAGCGAGCAGGGCGCG |
| | | GGCTGCGGGAGTGGGGGGCACGCAGGGCACCCCGCGAGCGGCCTCGCGACCAGGTACTGGCGGGAACGCGCCTAGCCCCGCGTGCCG |
| | | CCGGGGCCCGGGCTTGTTTTGCCCCAGTCCGAAGTTTCTGCTGGGTTGCCAGGCATGAGTG |
| 111 | chr4: | TGCGATCATTAAAATCAGTTCCTTCCCTCCTGTCCTGAGGGTAGGGGCGGGCAGATTTTATTACTTCTCTTTTCCTGATAGCAGAACTGAG |
| | 174664300- | GCGGGGTTGTGGAGGAGCGACGGAGGACCACCTCTAACTTCCCTTCACTTCCTGGATTTGAAGCCTCAGGGCCACCGGCCTCAGTCCTGTT |
| | 174664800 | ACGGTGGCGGACTCGCGAGGTTTTCCAGCAGCTCATTCCGGGACGGCGGTGTCTAGTCCAGTCCAGGGTAACTGGGCTCTCTGAGAGTCC |
| | | GACCTCCATCGGTCTGGGAGCGAGTGGTTCGAGTTCAGATGCTGGGAACCGTCGCTTCTCCCCGGCCGGGCTCGCTGTTTTCTCCTCCGC |
| | | TCGCCGTCATCAAGCCCGGCTATGAGCAGGGCTTTAAATCCTCCCTCCCTCACCCGCAGGTTTACCGAGCAGCCCCGGAGCTCTCAGACA |
| | | TGCTGCGCTGCGGCGGCCAGAGGAGGGGTGGGGGCATTGCCCTCTGCA |
| 112 | chr4: | GGGCTTGGGCCGCAGGCTTCCCTGGACTTCCGCAGTCCCCCTTCTCCCCATTCCAGAACCTGCCGAGCCCCTGCTGCATCTGGGACCCGC |
| | 174676300- | CTTCACCGTTTCCCAATCCCAGCGGTTAGCCCCTGCGCCCCCTTTTTGGTCTCCACTTTGCCGTTCGAAAATGCCTAGGTTGGTGGATCGA |
| | 174676800 | CCCTCCGCGGAGCAAAGACGGATGGCTGGCAGGAGCAGGTTCAGGAGCTGGGCCAAGGTATTCTCTGCTTCCGCCTTTGTGTCCGCCCC |
| | | CCCGCCCCCTGCTCCCCGCTTCCCGCCAGCATCTCTCCTTTTCTGCTCAGGAGTGTTTGGCCCGGCGGTCCACCCCGGCTTCCCGAGATA |
| | | CGCTAGAGTTGCCCCCACGTCCTGTCCGCCGCGCCCCTACCCACCGGGTTGCCTTCGGGGCCCTTCGGTGCTGTGTAGTCGGCGTGGCG |
| | | CTGTGAGCTAGGCGAACAGGAACCCCCAGGCCCGCCACGTCTACGCTATTA |
| 113 | SORBS2 | TTCTGGGGCCTGGATGGGTGCGAGCGGGACCCGGGGGAGTGGGAGTCGCCAGGCTCTGAGCAAGCAAGGGCTGCACCTGCACCTCTGC |
| | | CGGGCATGAAGAAAGGTAAGGAAGGAAGGAGCTCACCCGGGTGGGAGACAGAGCCGGGGCGCGCGAGCTTGGTGTGGGGGCGCCACTC |
| | | CGGGGCGGAGGGGAGGGGCTACCAGTGACTTCTCCGAGTCGGGAGCTAGAAAGAGGCTTCCGGCCAGGTTCCCTTGGAACAGGTGTCG |
| | | GAGTTGTTGGGAGAGGGGGCTGCAAGAAAGAGGGGTGCAGAAACTGGTTCATTAGATGGAGGCTCTGGGCGGAACCGCGAGGACACCCT |
| | | GGCAGCGCGCTGTGCCTGCGTTAGGCCGGGAGGGGAGAGGCCTCCGGACGGCGAAGTGTCCCTAGGGACCCAGACGCCTCGGGAGCG |
| | | ATCCGGGCCGCTGCGAAGCCCTGCCCACCAGGAGTGGATCCCCAGGATTCACCTCCCGGCTGCCTGCTCTGAGCTGAGAAGGGGATCTG |
| | | GTTCTTCACAATACCGTGGATGGCGGGGAAGGGGAGGGAGCCTGGGGTAAAATCCCATCTTGGTTTCCTCG |
| 114 | chr5: | TGTCACAGAAACCCCAGCAGCGCAGCCACCGGACTGGGTTCTGGAGGCCGAGCCGCAGTCCGTGCGGCGGCGCTGGGAAGAGAAGGCG |
| | 42986900- | CCCCGGCAGCTCCCCTGCCACCGGCCCCGAGGAGCGGCTGGCTCCCCCAGCCCAGCGCCGCCGCCGCCCGGTAACTCCAGGCGCAACT |
| | 42988200 | GGGCGCAACTGGGGCAGCTGCGACACCGAATCCCTCACATCTGCAACCTGGGTGCTGCGGCCACTGAGAAAATGGAGGCGCAGACCAAC |
| | | GAGCGGTGCCGCGACCGAGAGACCTCGGCTGGCGAAATGGTGGTGCCGGGAGCCTGCGAGTGACGCCAGCCGGCGGGGTTGTCAAGGA |
| | | CAACATTCGTTTTGACGCAGCCAATGGCGCCGTCACCAAGAAACCATCGACTCTGAGAAAAAAGAGAGGTTCGGCCACCGAGAAACTCCGT |
| | | ACGACAAGTGCTGTGGCAGAAAAACCGCCTACTCCGCGCCACAGGCAAAACAGCCAATGGAAACCCCAGGTGCTGCGACCGTGACACCG |
| | | GCACTAGAGGGTCTCGGATGGAGAAAGCGGCGCACGGAGACCAGGAAACTATGTGTAGCACAACTAGCAGAAAACCGTCTGGTCGGCCAT |
| | | CCGGGAGAAAGCGCGGATCAGAAACAAGCGACTTCGATGCAGGGAACCGCGCAGCCACTGAAGAAAGTGACCCACGTGGCAGTGGTGCC |
| | | AGCGAAACACTGCAGTTTGGACGGCAGCTGTGGGGATGCCACAGAGAAACATGCACTGCCACTGAAGTACATCCAGCTCCGCGGAGCTAG |
| | | TGTTCATATGATCAAGAAACCGCCAGTTGGGCTCTGCTAGAAACTTTTAGTCCTCCCTTAACGGCTATCCTACCCACAACAGACAATGCCT |
| | | TTACCCAGCACCTAGCGGTGCTGAGACCCGCCTGGGCCAGCACAGAGCGCAGAGCAGTACGGGTACGGAGAAACGCCGGACTCAGTGAAAC |
| | | CAGCCTTGCCTCCAGCGGATTCCCCGGCTTCGCCGGACGCCACAGGCAGAGTGCCGCGGGGAAACCTCTGGCTCCCTAAACCGATTAGA |
| | | TTGTGGGAGTGGGGGGGACACTCACAAGTTGTGTGGAAGGGAACCAGCGGCAATGGGACCCGGCGAGCACTTGCCCGCAGCAAATGCCT |
| | | GCGCTGCTGCAAAAAAAACAACTTTTGGCGCAAAGAATGTTGCGGCCAGAGAGCATCCGCTGTCGCTGACAAAGGAGTAGCAATGGCAAT |
| | | GAGAAACCGCCGGCGCCACGGCCGACCGCGGCGGCTCACGCCTATGAT |
| 115 | chr5: | CAAACGCTGAGAGACAAAAAGACACCAACACCCACCAGGACTGCGTCCTGCCAGCTCTTCACTCCGCTGACCTGACCTTCCACGCCCCTA |
| | 72712000- | GTCCTCGAGCGGACTTGACCTGTGGGGGAGTACCGAACCGTCCCCATGAGGCCCTCCAAGCGGCCAGGTGGCCTCCGCCACTCTCTCCA |
| | 72714100 | CCCCCACCTCCTCCACCCCCCAGCCCATCGGTCCATCTTCGATCTGCAAAACACGCCGGGTCAGCGACGCATCGGTCCCAGGCTTGTGAC |
| | | CACCTCTTTCTCTGTTACTTGGGGAGCCAGGCCCACCGCTCAGGATCACAGTGAGGAGAAAAAAGACACAAACGCCAGGACAGGGCGGCT |
| | | GGGGAAGGAAACTGCTAGGGACCGCTCATTGTCAGCCTGGCGTGTCCCACGGATCGCAGGACCCGTCGAGGCTTTGCTCTCTGCGACCC |
| | | GAATACTCCTGGGCCTCTCGACCTCCTCCTCGGACTCAGGCGTCCGCGTCTCCGGTCATCACGGGAGACCAATTGGTTTACAAATAGTGAT |
| | | GATAAACCTGGGACCGACCTTGGGGCTGTGTAAAAGTCTACTGACAGATGTAATGGAGGGTTGTTAGCAGTCACAAAGCCTGTCGGACCCG |
| | | TAGCATTAGTTCAAGAGACTATTTTCGTGTCGCACCAAAATTACTGCGCGTGTAAACCAATTTCCCCGACGGAAGAATAAACAGAGATTCG |
| | | TTTGAAGCGCGAGATGAAAACAGATGGGGTATCGCAAACAGTTCCCCAAAATACAACAGACTTCTGGGCCAATTACACGTGGTTAGCTCTG |
| | | AATGGCAGAGGAAATAGTTTTCTTTGCTGCTAAATGTCACAAAAGTCACCTAAAGGCACAGAGGAGGCCGCTCTGTTTTTGCGAAACTTGC |
| | | TAAAATTAATCTGCGCTGGGCCACTTGCAGAAAGCAGAACCACCTCCCGCCCCCACCTCGCCTCCAGCCGCCGGGGTTCAGGCGTTTGTGA |
| | | AAGACAGAACCTTTGGGCTAGGGACCCGGGCACTGGTGCTTCGAAGTCCGAATCCGCCGGCCGAGAAAACGACAAGAGAAAGAAAATCCAG |
| | | CGGGCGCTCTCTCCAGCGCCAGGCCGGTGTAGGAGGGCGCTGGGGCTCGGCCTGCCACCCCTACCCGACATTGGGAAGCAGCCCCTGCG |
| | | CTCCCGCGGCGCCTCAGCCTCCGGTCCCCGCCCCGAGGTGCGCGTTCCTCCTCCCGCATGCCCGTCTCGGGCCCCACGGAGCAAGAAG |
| | | ATAGACGATGACGAGGCGCGCCCATCCATCCGGGCCGACGAGGTCAGGCCCGCGCCACAGGCAAAAATTGCGCAAGCCCGGCCGCAGG |
| | | GATTTCGCGGGCGCCTGGGTCCCAGGTGCGCGGCCGAAATCCTCAGGGAAAATCCCGAGGGGCCAACGGTCTAGGCCACAGGGCTGCT |
| | | GGGCCCGGGCCTGGCTCAGAGCGCATTCGGGCGGGGAGGCCGCACGCCGCACCCGGGCCTCTCCTCCGAGCCCGAGGCAGGCACTGA |
| | | GCTCCGGGCCAGCCAGGTGCCTCCCGGCTGGTGCGAGACCCCGGGCCTGCTGGGAGGCGTGGGCAGGGCAGGGCAGGGCTGAACCCC |
| | | AGCGACTGAATCTCGAAGGCAGGAGGCCTCGGAGGTCATCGGCCCAGCTCGCCTGAAACTGTCCCTGCTCGTGCCAGGGCGCGGGCAGA |
| | | GGAGAAAGGACAGGGCGGAGCAAGCCCACTGCAGAACTGCGGTCGGTGGCTGCGAAGGGTCCGGGTCACCGCGCTCCCGGACGCCGGA |
| | | AGCCGCGCTGGCGGGGCCGCGGGGAGGGAGGCTGGGTACCGGGGCCGTCCGGCCGGAGGAAGCGGCTCCGGCCGCGCTGTCCGCGC |
| | | TTGGGAGCCGCGTGCAGGGTTCAGCCGTGTTTCAGTTGCCCTCTGACCTGACCCCGGGCGCACAAAGGCCTCCCGGGTGCGCCGCCATG |
| | | GCCCAGTCTTCCAGTCGCTGCCAAATTAATGAGCCCACGTCAGGTTGGGTTTACAGCTCGGCCGGGAAGCAGCCGAGTGGAAAATGAGCT |
| | | CGGGGCCGCTCCAGAGGCTCCCGCACAACTGCAGAGGCTGCCCGCG |
| 116 | chr5: | TTTCCAAGACAGAAGGAGGGAACTAGGCGCCTTTTTTCCACTCCGCTGACCCCAACGTCTGGGCTGTGCGTTGTAACGCAGTTGGCGGGG |
| | 72767550- | CCTTCAGCTTGGGATGAGGGCGAAGGGGCTCGGGATGGGTGGGAAAGCAAGGACCGGGCAACAGGTGGGGAGGTGGCGGACTTTTGTC |
| | 72767800 | TCGGGGAAGGAAATCGGCTGTGCTGAAAGGGCGGAAAGCAGTAGCGCACAGAACTAGTGTCTGCGGGGTCCC |
| 117 | NR2F1 | CCCTCCTGTGGCTGCTTGGGCAGACGCCTGTGGCCTGTCGGATGCGGCCCACATCGAGAGCCTGCAGGAGAAGTCGCAGTGCGCACTGG |
| | | AGGAGTACGTGAGGAGCCAGTACCCCAACCAGCCCAGCCGTTTTGGCAAACTGCTGCTGCGACTGCCCTCGCTGCGCACCGTGTCCTCCT |
| | | CCGTCATCGAGCAGCTCTTCTTCGTCCGTTTGGTAGGTAAAACCCCCATCGAAACTCTCATCCGCGATATG |
| 118 | PCDHGA1 | TCCTCCTTTGTGTATGTCAACCCAGAGGATGGACGGATCTTTGCCCAGCGTACCTTTGACTATGAATTGCTGCAGATGCTGCAGATTGTGG |
| | | TGGGGGTTCGAGACTCCGGCTCTCCCCCATTGCATGCCAACACATCTCTGCATGTGTTTGTCCTAGACGAGAATGATAATGCCCCAGCTGT |
| | | GCTGCACCCACGGCCAGACTGGGAACACTCAGCCCCCCAGCGTCTCCCTCGCTCTGCTCCTCCTGGCTCCTTGGTCACCAAGGTGACAGCC |
| | | GTGGATGCTGATGCAGGCCACAATGCGTGGCTCTCCTACTCACTGTTGCCACAGTCCACAGCCCCAGGACTGTTCCTCGTGTCTACACACA |
| | | CTGGTGAGGTGCGCACAGCCCGGGCCTTACTGGAGGATGACTCTGACACCCAGCAGGTGGTGGTCCTGGTGAGGGACAATGGTGACCCT |
| | | TCACTCTCCTCCACAGCCACAGTGCTGCTGGTTCTGGAGGATGAGGACCCTGAGGAAATGCCCAAATCCAGTGACTTCCTCATACACCCTC |
| | | CTGAGCGTTCAGACCTTACCCTTTACCTCATTGTGGCTCTAGCGACCGTCAGTCTCTTATCCCTAGTCACCTTCACCTTTCTGTCAGCGAA |
| | | GTGCCTTCAGGGAAACGCAGACGGGGACGGGGGTGGAGGGCAGTGCTGCAGGCGCCAGGACTCACCCTCCCCGGACTTCTATAAGCAGTC |
| | | CAGCCCCAACCTGCAGGTGAGCTCGGACGGCACGCTCAAGTACATGGAGGTGACGCTGCGGCCCACAGACTCGCAGAGCCACTGCTACA |
| | | GGACGTGCTTTTCACCGGCCTCGGACGGCAGTGACTTCACTTTTCTAAGACCCCTCAGCGTTCAGCAGCCCACAGCTCTGGCGCTGGAGC |
| | | CTGACGCCATCCGGTCCCGCTCTAATACGCTGCGGGAGCGGAGCCAGGTGAGGGGCTCGGCGCCGCCCCGGGCGACCCCTGGGGGCG |
| | | GCACTGGAGAAGCCGCCCGTCCTCATAAGGGATTGAACTTGCATCCACTCCTCTCCGGCCGGCTTGGTCGCTGGCTGCGCTCCACCCGAT |
| | | TCTCGGGATCATTGGACCGTTTGCGCGAAACCAGAGTGGCCGATTAAGGGATGGGGCTCCGAGCACCGGGGGTGGTGGCGACTGTGGGC |
| | | GAGGGGAGGTGGGACCGACCCCCACCCCTACACTCAAAAAAGGCCGGGGCCTCCTTCGAGCTTCCGGTGAATTTCGGGCGATTTCCGCG |
| | | GGTGTCGGGGGTCCCGGGAGGAGGCAGTCACAGATCCACCCCTGCAGCCAGCCTCCTAGGCGCCGGCTCCGGCACGCTTCGCCGGTCTGT |
| | | AGATTTCCTCTTCGATTTCTCCCCAGCTCCCAGCATCTGTGACTTCACTGTTACCCTCCCTATCCCCGCATCACCCAACCGCACCTGTCT |
| | | GCGGGACTTAGGTGTGCGCGCGGGGCTCATGCGTGTCCTCCCTGCTGGCCACCCCCACGGCCCACACAAGTTGCACGGGCTCGCCACGC |
| | | CCCGCCAACACGTGCGCGGACGCACGCACGCACTCCTCGCACGTGGGCTTACGCGAATACCAGCTTTCACTGCCACTCGCTCGCGGCCA |
| | | GATTCACAGGCCTGTTCCGGTCCACTCGCAGCTCCCCTCTGCCGCTCCCTCCGCCGGGCTCAGGAGTACTCGTAGCTGATTGTGCGCGCC |
| | | TGAGGGTCCCAGATCGCGGCCGCCCAGGACCAGGCGAGGACTCCGGAGCCTCCTCTCACCTCTCCCACCTGCGCCCCGGGCTGGGCCG |
| | | GGTCGCCTGGGGGGCGGCCTGAGCGAGGCGCGGGGCCAGGAGCGCTGGAGCGACTGCCGCTCTAAGTGCCGGGCGGGCAGGACTCTA |
| | | CGATCCTTGGGCCAGAGGTCCGGATGGTCCCGGGACTCCGTCTCAAGGGTCGGCGACCCCTCAACCCAGAAGCCTCGAGCAGGCGGACA |
| | | GGCAGAGCTGCCCAGTGGCCGAGGCGCGG |
| 119 | chr6: | ATTTGTCGTTGTGCCATTGCTGCCACTGTTGTTCTTGTCCAGGGAAACACCGGTGGCCAACCCAGATCGGATACAATGGTGCGGCTCTGGA |
| | 10489100- | CTGAGCCTCCAACCACATTAGCCATGGGCAGCATTGTTGCTGCCGCTGCTGTTATTTTAATTATGATTGTACGTTAACCACCACCTTCCTT |
| | 10490200 | CCTCTGCCTCCCTTCAGCTGCAATGATGTATGTTACTTTTTGGTAACTGGATTTCATTAACATTTATGAACTCTCATAAAGTAGTAGAAAA |
| | | AGCAATTTGTGTGGAAGAATTTTCCACCTCATTAAACAGTGTTCTTTTGGGGGTCAAGCTGATATTTTTTTTGTTGTTAGATTTTTTTTAT |
| | | AGGTCCTTTGTCCTTCCCTAAGCCCTGGGGGATGAAAGGAGAGCCGTCCACCCAGCGAGGGGCTTGTGTGCCCTAGAGGGCGCTGGGCCCC |
| | | GCGCGCTTTCCTGGCTGTCCCCGCCGGCTTTCCACCCTCCCCAAAGCCCAGGTGCCCACCGTGGGTCGCTGCGGCCTTTCCCCTTCTTGGC |
| | | CAAATCCGATTACTTCGCAGCCTGCAGATGGCATCGCCGGCTAAGGGCAGCCTGCGGCAGGTCCCCGAGCCTGAGCACTCCTCCTATCTGG |
| | | GGCCTGAGAGGACGCTCTGGGCTTTTTCCCAGGCCCAGGGTGCGCGGCCTGCTAGCGCCTTTCGAGGCACAGTCCCAAGATAGGCTCTTGT |
| | | CCTTCGACGCCCCCTTGGCACAAGCGCACTGGCGCCCTCCGCTCAACCCACCTTGCCTTTGGGGCGGGCTTCAACCCTGGGAAGACAGGCC |
| | | TGGGGGAAGCGAGAGGAGAGGCCCGAATAGAGGTTCCGGCTCAATCTTTCCCAGACGGAGGCCTGGTGTTTCCAGCTCAGTTGCATCTTC |
| | | CAGCCGCGGGCTCCTGGCCCAAACAGAATGTGTTTGCTTTCACACCGGGACGGCAAGCGGAGTCCGCCTCAGTGAGCAGCGAGCTGCGC |
| | | AGTCCGGACGGGTGTCGCCCCCAGAGACTCGCCAGCCGCCCCCAGACACTCGCCAGCCGTCCCCATCTCTAATCCACCGTCCAGGCCCG |
| | | GGCCCTGGGAAGA |
| 120 | FOXP4 | CCGTGTCTCCCTTAAGAACTGGGGCCTCATCTCCACTCCAGCTGCGCGTGCACGTGTGCTCCCGGCAGGACGCGCGCCCAGGAGCGCGC |
| | | TGGGGGCTGCCCCGCCCCTCTCTCCCTCCCCCGCGGGTAAACTCCGGGCATCCATCAGTCTGTTAATTGCACTAATTAGAGATCGCAGAG |
| | | GTGTTAATTGGAAAACCCTGGTATTGTGCCTGTTTGGGGGAAGAAAACGTCAATAAAAATTAATTGATGAGTTGGCAGGGCGGGCGGTGCG |
| | | GGTTCGCGGCGAGGCGCAGGGTGTCATGGCAAATGTTACGGCTCAGATTAAGCGATTGTTAATTAAAAAGCGACGGTAATTAATACTCGCT |
| | | ACGCCATATGGGCCCGTGAAAAGGCACAAAAGGTTTCTCCGCATGTGGGGTTCCCCTTCTCTTTTCTCCTTCCACAAAAGCACCCCAGCCC |
| | | GTGGGTCCCCCCTTTGGCCCCAAGGTAGGTGGAACTCGTCACTTCCGGCCAGGGAGGGGATGGGGCGGTCTCCGGCGAGTTCCAAGGG |
| | | CGTCCCTCGTTGCGCACTCGCCCGCCCAGGTTCTTTGAAGAGCCAGGAGCCTCCGGGGAAGTGGGAGCCCCCAGCGGCCCGCAGACTGC |
| | | CTCAGAGCGGAAGAGGCAGCCGCGGCTTTGACCCAGCTTCCTTCCGACGGCATCTGCAGGAGCCTCTAGGCCTGACATAGGCTCCGAGG |
| | | TGCCCTGGCTCCCCCACGGGGAATGCTGAGGGTTGGGCCACTAGGTCCTGCCTAAGTGCAGGACCTGAGCCTCAGACAAATC |
| 121 | chr7: | GGGATTGCCGGCTTTGAGAAAATATGAAGAAACCGATTTCTCCTTCCACTTTGCCAGTGCACTTTCCTTCCACTTTCACTGGTGCTGGGGG |
| | 19118400- | CGGCGCACTCTTTACGACATATAAGCGGAAAATTCTGCAAAAGTGGCCCCCGGGGATCCCCGCCCGACCCCTGTCTGTCGCTAATGTGGGC |
| | 19118700 | CTGTCTCCGGAAATTCGAGGTTGGGCCTTTGCCTGAATCTGTTGCTATTGCTCCCCTTGCTACCGCTGACACTTGGCACCGCCGCCTCCTA |
| | | GCAGCGGCCAGACGCGGGGCTGGGGGC |
| 122 | chr7: | GTTGCGAGCGCGGCACAGGTTGCTGGTAGCTTCTGGACTCTGGAGGCTTGGCCTTCCTTCTAAGCCGATGGCGGGGAAAGAACCTCGTTT |
| | 27258000- | CCACAGCTTCCCCGACCCCCGCCGCTTGCCATTTGGGGACGGGAAGCGCGCCCGGGTCGCTTCACGTCCCTCTGGGCCGGAGCCCTTTC |
| | 27258400 | CATGGCTGGCTCCTCTGGGGGCCCTTGGGCCTGTGAGCAGCGTCTACTTCCCTCAGAGAAGAATCCTTTCCTTCCCCCATCGAAGTGTCCC |
| | | TTTCTGTATCCTGAAATAACCCCTCCTGGGTGAGGCCAGTTCCCCTCTGTCGCCCTCCTCCCGCAGGCGTCCGGGAGCCTCGTGAGGACC |
| | | CCGTGCAGTTGAGTCCAGGCGACAGGTGCCTCCCCAGGTG |
| 123 | TBX20 | CAGTGCGCCCCTTACCGGAGCACCCATGGCCTCCCGCGTTACCCCAAATTTTGTAGGCAGACTGTCAGAGTTCGAAGCCAGCTGTGTCCT |
| | | CTGCGGGCCGTGTGACCCTAGGCTATCTGGGCTGCTCGGAGCCTTAGTTTCCCTAGTTGTGAAGAGGGAGGGTGTGACCATGGCCCGGA |
| | | GCTCTCCGAAAGGCTGTGCGGATTGCTCGGTGGCGGGATGTGGAGCGCGTCTTCTATGATGCCAGGTGCTGGCCAAGCGCTCGATGCAG |
| | | GCTGCTCCAGTTAGGTCGATGCGATGGCGGGAAGCACTTTCCTCTGCAATGGAGAGACGCCGACACCCCGAGCCCGAAGGCTTGCAAGG |
| | | CGCGCTCTCGCCACTGGGGTCGGGGATCCGTGGGTTCTCTATCCCGCTTACCCACTCCATCCTTAGCAGCTGTCGTCGGTCCCAGACCTC |
| | | TACCTTGGAGAGACCAAGGCGGCCCAGAGCCCAGGAGACTACTGCGCGGTACGCCAGGATCCAGAAGTGGATTCTGACTTCTAAAGACCC |
| | | CTCCCAAGCCAACGCTATCAGGGTCCCTGCAAGCGGTTGACTGTGGCGGAGGCAGAACCAAAACCTTTGCTCTGCCCGCGGCGCTCCAGC |
| | | CTCTCACCCAGGACAGTGCTCTGGGCTCCAGCCGCTGCAGTGGGGTCGGGACACAGACGCCGAGTTAGAAGCCCCGCCGCTGCAGGTCC |
| | | CTGCTTGGTCGGCGCGGTGACGGTGTCGCTGGCGGCGGCGGGGGCCTTCCTTTGGCTGCCCGGCCATTTAATCAGAGCTATTAT |
| 124 | AGBL3 | TTTAGTATTTAAGGAGAAAAGCCTCATTTTCCAGAATCGAATAAGCGAATTAATCGCACAATTGTGTAGAATGGAACTCAGTCTGTAAAAA |
| | | ATCAAGACCAACGTACTTTTTAATATTCTAACATCTCCAAGTAGTAGTTACAAGTATTGTACCCATGAAGTCCAGGTAATTAATTTGTTCA |
| | | ATGTCACACTGTTAAAAGTCAGGTGGGCTCCAAAGCACAGTCCTAACCAGCATGCTCTACTGCCTCCTCTGAGGCAACAGCCGAAGTGCAG |
| | | ACCACTGGGAATAAATAGCTGCCCGGTCTTCCCCACTCCTAAATTCTCCCGACAGACCCCAAAGCCTCTCTGAGAGCCTCTCTGACCGCCC |
| | | TGCGGCCCACCCCGAGTTCCCGGCATCCTCTGGGATCCCTCTTCCTGGAGCCAAAACCTACGCAGGCTCCTTTCCTCCGAGCTGGTTGCTA |
| | | GGTGATCTCCGAAGGCTGTCCGAAGTCTCGCGAGGGCGGACCCGTTGCCTGATGACGAGAGTTGGGAGTGTGGCTGGGGCTGCGGATCTCC |
| | | AGCAGTGGCGTTACTTCTAGCGGCTGGATACCGGGTTCTCCGCGAGATCGCGAGATCCCGAGATATTCTCCCCGCACGGAAGCGACGACTG |
| | | GCCTGGCCAGAGGACTCGCGTGGGAGCGAGGTGCCGGCCCCGACAGGACGGTGAGGTATGCAGAAGTAAGGCGGGGCGCCCCCTGCGG |
| | | GAAGCGAGCGCGCCCCGGAAAATGAGCGCCTCCCCACACCAAGGTGTCCAGGAGTGAGTGCGGGAAGGAACTCGGCCGCCCGGAGTTG |
| | | TGGCCTCATCGTGCTTCCCGCCAAAAACGCCTTGGTACTGTCGGGACGCGGCTAAGCGTGGACGCGCCCGCATCTGCCCCTCCTCCGCA |
| | | GTGGTGGAAGACACCCGCGGAGCGCCGGTGGATAAGGGCCGTTTCCTGAGACCAGAGCTGTATCCGCAGCAGGTCAGCACTTCGTGCGC |
| | | CCTGTGTGC |
| 125 | XPO7 | AGCGGCGCTGTTCCCGGGCTGGGTGCAGCTGCTAAGGACAAGGCCCCTGCTCCGAAGAACGCGGTGGCTCGGGGATACCCTGAAAGGG |
| | | ACGGCCATGGCGCACATGGGATGCCCTAGGGTTCGTGGGAGGGCATGCAGGCGCAGCCCCCGCAGGGGTTGGCCTGCCAGAGAAGGCA |
| | | GGGGAGAGCACTCGGGGCTGCACAAATGGTGTGGCCGGAGGGAAGGTGCAGCCTTGTGTGTGTCTGGATGAGGGCTGGGCATAGGAGC |
| | | TTGGTATTTGATCCTGAAAGCTCTGCGTTTCCAAAG |
| 126 | chr8: | GAGTCATACTTGTAGTCACATCCTTTTCCTTTCTCCAACCCACTGGTTAATCATGAAAGGCTCTTCTGATTGGCTGCCTCCTGGCAGTAGT |
| | 41543400- | CGCTCAGCGCGACGGTTCGGGAGCAAATAAATAATTCCCGCTGGGAAGCTGTTTCTCAGACAGGAGCAGCGACACCCCTGCCACGCCTGCC |
| | 41544000 | GCCTGGAGTTGAGTGGGGTAAGCACGCCGGCCTCCAGGAATCGACGGTGCCACGTGGTTCTTCTTGCACTTCTCTTCTTCTCCAGTTTCAG |
| | | GGGACACCGTGGGGTGTGCGAGCCCGGGGGAGCGCAGGGAAGGGCGGGTTGGGCTGCAGGTGGGAATGTGCGGTCCTTCTGCGCCCTCA |
| | | ACAGAGCTTCCTTCCTTTTTGCCAAGGTCCCCGTGCCGCCTTCAGCGCGCCTCCTTATGCACCTCTACCTCTGCTGCAGCGTACCTCTTCC |
| | | GCAGCCCTAGCGGCCTCCCCGAGGGGCGCCGCGGCCTCGGCTGTCCCTCCCCTGCCTGGCACGACCACCTGACCCCCAGCGACCCAAG |
| | | AAGCAAGTTGTGTTTGCAGACGCAAAGGGGCTGTCGTTGGTATCGGTGCACTGGTTTGA |
| 127 | GDF6 | ACACTTTCTGTGTGGGAGGGCACAAGACATGGGCTATGACATGGCCAGAGACCCCACCTTCTTTACACATGTAAAAACCAACCAAATCAAG |
| | | ATGCGTCAACGGTGATTCTTCCTCCCACATTGTTTCCCTTTTTAAACTGTTATTTTTTCAATCCATGGAGCAGTTGAGAAACGGGTATGCA |
| | | TCTCTCCTCCCCTCCCCTTCTATCAAAGCCTGTAAGACACATAAGGAAATCCAAAGCCACAGTAATAGAGAGAGAGAGAGAGAGAGAGAGA |
| | | GAGAGAGAGAGAGAGAGAGAGAAAACAGAACAAAAGAAATCCTCCTTGGCTTGTTTTTCCAGGGTGGCCAGGCAAGGTGTGAAAATCCATA |
| | | TTTCCCTCTGGGCTGGCAGGTAGAAGTTACTGGGAAGGCTGCGCTCCCTTCTCTCCCACCGGCTCTCACATCCAGGCTGTTCCCTCACCCT |
| | | CAGCCTCCCCCAGCGCCAGCTTCCTCCTCCGCCTCTCTGCAGCCAGGCCTCCCCTGCAAGGCGGACCTTGGCCCACCTTGGTTCCGGGCCA |
| | | AGGCGGCGGGAAAGGCACCGCTACCTGCAGCCGCACGACTCCACCACCATGTCCTCGTACTGCTTGTAGACCACATTATTGCCCGCGTCG |
| | | ATGTATAGAATGCTGATGGGAGTCAATTTGGTGGGCACGCAGCAGCTGGGCGGGGTGGAGCCGGGGTCCATGGAGTTCATCAGCGTCTG |
| | | GATGATGGCGTGGTTGGTGGGCTCCAGGTGCGAGCGCAGCGGGAAGTCGCATACACCCTCGCAGTGATAGGCCTCGTACTCCAGGGGCG |
| | | CGATAATCCAGTCGTCCCAGCCCAGCTCCTTGAAGTTCACGTGCAGGGGCTTCTTGCTGCAGCGTAGCCTGGACTTCTTGCCGTGCCGCTT |
| | | GCCATGGCGACTGGCGAAGGCCGTGCGCCGCCGCCGGCGGCCGGGCGAGGGCAGCCAAGGCCTGGCATCCGGGGCGCCCGACGGCG |
| | | GCGGCCACGACCCCTCGGCGCCCGCGCCCGGGCCCGCAGCCTCGGCCGAGCCCAGCTGCTCGCGCATCTCTGCGAACAGGTTCTTGCG |
| | | CTGGGATCTGGTGAATACCACCAGCAGGGCCCGCTCCTGGGGAGGCCGCACCCTCCGGCCGAAGCCCAGACTCCGCAGGTCCGGGGGC |
| | | GGCGGTTGCTGGGGTCCCCGCGCGCGCGCCTCGGCCTCCCCGGCGTCCAGCTCGCCCCATGCGGCCCGCAGCTCCAAGCACAGCTGCT |
| | | TCCAGGGCTGGTGGCGCAGGCCCTGCCACACGTCGAAGACTTCCCAGCCGGCCGGCGGCGCCCCCTGCGGGTCCAGGGTCCGCGCGTC |
| | | CAGCAGTAGGGGCGAAAGGCAAGGGAAGAGCTGCACGTGGAGCGGCCCGGCTGGTGGCCCCCAGGGCGCTGAGGGCGCCTGGCGAAA |
| | | GAGCCGCAGCTCCGCGCCCACCAGCTCTTCTTTGTCTGAGAGCATGGACACATCAAACAAATACTTCTGTCTCCGGAGAGGAGTGTGCGA |
| | | GAGATCGTCTGCGAGATAAAAAATAATTACAGTCAGTTTCACTTAAGGGGGAGATCAGCCCGGTGCTCTTCGGCCGCCCCGGGAGGAAAA |
| | | GGGCGGGGAGTGGGGGCAGGTCGGCCGGGCAGTCCAGCTTGCCCGGCCCAGGGCCTGACCACCCCGGCTCCCCATCTGGCTGGTGCAT |
| | | GG |
| 128 | OSR2 | GCCCGCTGTGAATGTAGGTGAGGTGATCCCGGGAACCTGGGTCTGAAATCAGACCTGTGTTGCCATTGGGAGCACGGAGAGAGGGGAAG |
| | | CGCCCTGCTTAGGCCCAGGCCGGGCGTCCTGGTGGTGGGACCGCAGCCGCACTCACCTCCAGGCCAACGGACAAGGTTCCTGCAAGCCA |
| | | GCAGGGCCACTCTGTGCTTGGCCTACTGCAGCTCCCCTGCAGCTCCTTTCCTCTCCCTCCCCGGAGCGCTCTCCTCTCTCCTCTCCCCTCT |
| | | CTTCTCTCTCCTCTCTCGTCTCCTGGGGCATCCCGGGTGGAGGGATGTAGGGGTCGCTCCTCGGTGCCAGGCCGGGAAGCAGCTCAGGC |
| | | CTCCCAAGAGCTTGGCGCTCAGTCTGGGAAAAGGGGTTCCTCTGGCCTCAGGGACGTTCTCCGCCCCCACCCCACCCCCTGGGAGCCTG |
| | | AACCATCTGGAAGGGATCTTAGTCGGGGGTTGGGAGGAGAGCCCGTGGATAGGAGGAGGGGGCGATTCTAGGCCGAATCCAGCCCCTGA |
| | | GGTGTCACTTTTCTTTCCTGCGGCCCGTCACCGCTGATAGATGGGGCTGAGGGCAGAGGAAGGAAAAAGAAAACCTCCGAGGTCAGTGCG |
| | | GGGCGAGGTGAGCCCCTCCCAGGGCCCTCTGGCCCAGGAGGATGAAGCGCGCCGGCTTCGCTCTTGCACGCCGGCTTGCCATCCGGGT |
| | | AAGCGCGGGAAAGGCGGCCACAGGGCGCGGCGGCAGCGCAGCGCGTGGGATCTCACGACCCATCCGTTAACCCACCGTTCCCAGGAGC |
| | | TCCGAGGCGCAGCGGCGACAGAGGTTCGCCCCGGCCTGCTAGCATTGGCATTGCGGTTGACTGAGCTTCGCCTAACAGGCTTGGGGAGG |
| | | GTGGGCTGGGCTGGGCTGGGCTGGGCTGGGTGCTGCCCGGCTGTCCGCCTTTCGTTTTCCTGGGACCGAGGAGTCTTCCGCTCCGTATC |
| | | TGCCTAGAGTCTGAATCCGACTTTCTTTCCTTTGGGCACGCGCTCGCCAGTGGAGCACTTCTTGTTCTGGCCCCGGGCTGATCTGCACGCG |
| | | GACTTGAGCAGGTGCCAAGGTGCCACGCAGTCCCCTCACGGCTTTCGGGGGGTCTTGGAGTCGGGTGGGGAGGGAGACTTAGGTGTGGT |
| | | AACCTGCGCAGGTGCCAAAGGGCAGAAGGAGCAGCCTTGGATTATAGTCACGGTCTCTCCCTCTCTTCCCTGCCATTTTTAGGGCTTTCTC |
| | | TACGTGCTGTTGTCTCACTGGGTTTTTGTCGGAGCCCCACGCCCTCCGGCCTCTGATTCCTGGAAGAAAGGGTTGGTCCCCTCAGCACCCC |
| | | CAGCATCCCGGAAAATGGGGAGCAAGGCTCTGCCAGCGCCCATCCCGCTCCACCCGTCGCTGCAGCTCACCAATTACTCCTTCCTGCAGG |
| | | CCGTGAACACCTTCCCGGCCACGGTGGACCACCTGCAGGGCCTGTACGGTCTCAGCGCGGTACAGACCATGCACATGAACCACTGGACG |
| | | CTGGGGTATCCCAATGTGCACGAGATCACCCGCTCCACCATCACGGAGATGGCGGCGGCGCAGGGCCTCGTGGACGCGCGCTTCCCCTT |
| | | CCCGGCCCTGCCTTTTACCACCCACCTATTCCACCCCAAGCAGGGGGCCATTGCCCACGTCCTCCCAGCCCTGCACAAGGACCGGCCCCG |
| | | TTTTGACTTTGCCAATTTGGCGGTGGCTGCCACGCAAGAGGATCCGCCTAAGATGGGAGACCTGAGCAAGCTGAGCCCAGGACTGGGTAG |
| | | CCCCATCTCGGGCCTCAGTAAATTGACTCCGGACAGAAAGCCCTCTCGAGGAAGGTTGCCCTCCAAAACGAAAAAAGAGTTTATCTGCAAG |
| | | TTTTGCGGCAGACACTTTACCAAATCCTACAATTTGCTCATCCATGAGAGGACCCACACGGACGAGAGGCCGTACACGTGTGACATCTGCC |
| | | ACAAGGCCTTCCGGAGGCAAGATCACCT |
| 129 | GLIS3 | CACTCCCCCGCCGCCTCCGCCCCTAACCCTCGGCCCCGTGCGCGAGCGAGCGAGGGAGCGAACGCAGCGCAACAAAACAAACTAGTGCC |
| | | GGCTTCCTGTTGTGCAACTCGCTCCTGAGTGAGTCGGGGGCCGAAAGGGTGCTGCGGCTGGGAAGCCCGGGCGCCGGGGACCTGCGCG |
| | | CGCTGCCCGGCCTGGCCGGAGCCTGTAGCCCGGGGGCGCCACGGCCGGGCTCGCAGTCCCCCCACGCCGGCCCCCCGGTCCCCGCCG |
| | | AGCCAGTGTCCTCACCCTGTGGTTTCCTTTCGCTTCTCGCCTCCCAAACACCTCCAGCAAGTCGGAGGGCGCGAACGCGGAGCCAGAAACC |
| | | CTTCCCCAAAGTTTCTCCCGCCAGGTACCTAATTGAATCATCCATAGGATGACAAATCAGCCAGGGCCAAGATTTCCAGACACTTGAGTGA |
| | | CTTCCCGGTCCCCGAGGTGACTTGTCAGCTCCAGTGAGTAACTTGGAACTGTCGCTCGGGGCAAGGTGTGTGTCTAGGAGAGAGCCGGCG |
| | | GCTCACTCACGCTTTCCAGAGAGCGACCCGGGCCGACTTCAAAATACACACAGGGTCATTTATAGGGACTGGAGCCGCGCGCAGGACAAC |
| | | GTCTCCGAGACTGAGACATTTTCCAAACAGTGCTGACATTTTGTCGGGCCCCATAAAAAATGTAAACGCGAGGTGACGAACCCGGCGGGGA |
| | | GGGTTCGTGTCTGGCTGTGTCTGCGTCCTGGCGGCGTGGGAGGTTATAGTTCCAGACCTGGCGGCTGCGGATCGCCGGGCCGGTACCCG |
| | | CGAGGAGTGTAGGTACCCTCAGCCCGACCACCTCCCGCAATCATGGGGACACCGGCTTGGATGAGACACAGGCGTGGAAAACAGCCTTC |
| | | GTGAAACTCCACAAACACGTGGAACTTGAAAAGACAACTACAGCCCCGCGTGTGCGCGAGAGACCTCACGTCACCCCATCAGTTCCCACTT |
| | | CGCCAAAGTTTCCCTTCAGTGGGGACTCCAGAGTGGTGCGCCCCATGCCCGTGCGTCCTGTAACGTGCCCTGATTGTGTACCCCTCTGCCC |
| | | GCTCTACTTGAAATGAAAACACAAAAACTGTTCCGAATTAGCGCAACTTTAAAGCCCCGTTATCTGTCTTCTACACTGGGCGCTCTTAGGC |
| | | CACTGACAGAAACATGGTTTGAACCCTAATTGTTGCTATCAGTCTCAGTCAGCGCAGGTCTCTCAGTGACCTGTGACGCCGGGAGTTGAGG |
| | | TGCGCGTATCCTTAAACCCGCGCGAACGCCACCGGCTCAGCGTAGAAAACTATTTGTAATCCCTAGTTTGCGTCTCTGAGCTTTAACTCCC |
| | | CCACACTCTCAAGCGCCCGGTTTCTCCTCGTCTCTCGCCTGCGAGCAAAGTTCCTATGGCATCCACTTACCAGGTAACCGGGATTTCCACA |
| | | ACAAAGCCCGGCGTGCGGGTCCCTTCCCCCGGCCGGCCAGCGCGAGTGACAGCGGGCGGCCGGCGCTGGCGAGGAGTAACTTGGGGCT |
| | | CCAGCCCTTCAGAGCGCTCCGCGGGCTGTGCCTCCTTCGGAAATGAAAACCCCCATCCAAACGGGGGGACGGAGCGCGGAAACCCGGCC |
| | | CAAGTGCCGTGTGTGCGCGCGCGTCTGCGAGGGCAGCGGCGGCAGGGGGAGGAGGAGGCAGAGGCGGGGTGGCTGGACCCTCGGCATCA |
| | | GCTCATTCTCCCCTGCTACACACATACACACACAAATAATGTTTCTAAAAAGTTCAGTTGCGACTTTGTGCCTCGCCTGTCCTGTTCATCC |
| | | TCGTCCTGGGCCGGGGAATGCTTCTGGGGGCCGACCCCGGGATGCTGGCTAATTGCTGCCGGCGGGTTCCGTCGCCGGTGTGACCCTG |
| | | GACGGCGCGGACGGCGTACAGGGGGTCCCGGGAGGGGCAGTGGCCGCGGCACTCGCCGCCGGTGCCCGTGCGCGCCGCGCTCTGGG |
| | | CTGCCCGGGCGGCGCAGTGTGGACGCGG |
| 130 | NOTCH1 | CTGAAAAGCCGTCAGGGAAACCACACATGTTCAACCCCTGGCGGCTCCCCCAAACCTCTCATTTCCAGTAACTGTGTGTTTCCGCTCGTC |
| | | AACAGCTGAAACCGAGCGGAACTTGGGGGGCCCCACCACGCGGCCCTGCTGTGCGGCACGGGGCTCATCTGTCCCCCGGCTGCGGGGAG |
| | | TCAGCTCTCACCGCCCACCTCCTTCCCAGATAGTCTCTGTGCCCACTCGACGGCCCGGCAAGCCCAGCCCCTGCCTGCCACGGCCACAGC |
| | | AGCCTCAGAGAGCTGCCCTCTCTGGCCAGGGTCAGGGCCTGAGCTGCTGCCTCCCGCAGGGTCGAGGGCAGGACACTTGTCTGAGGCTT |
| | | GGGTGGGGCAATGGCACCTCCTCAGGGCCTCAGCCCCCGGGCAGGCTCGGTGACCATGGGCCTACAGCAGGGAAAATTCTGGGCCAAAA |
| | | GCTCCAGCCTCCTACTAGGGCATCTGTCTGCAAATGCACCTTAACCTGACCGCTTGGGCTGTGGGGGAGCCTGTTTCAGGGAAAGTGAGG |
| | | GACGCGCCAGTTTCCTCCTTTGGACTTGATGAGGCACGAACGCATCTCTAATAAAGCCAGGTCTCCCCGCCGTGGCTCCCTGGGCGGGTG |
| | | CCTGTGGCTCGGGCCATGAGTCACGCTGGGTAACCCCACTACGGGGAAGAGGGCAGGAAGCTGGGAGCCACCGCCTCTGTGCCCGGTTG |
| | | TCATCTCGGCACGAGGGCGACCGTCGGCTTCGTCCTGCCCTCATGGCTGAGGGCTTTTGGGATGTGGCGGGAGACGGGGGAGTC |
| 131 | EGFL7 | AAATCATCAGAATGGCTAAAATGAAAAAGACAGACAACAGCAAGTGCTGACAAGGGTGTGGGGCGGCCAAATGCTCCTGCACTGCTGGCA |
| | | GGGGACCTGAGAACTGCAGGGCATTCCCTGGCTTCCTGCCCCTCCTGGGACTGGGGACCCCCCAGGGACAGCCTAAGGGAACTGCATTT |
| | | ATCTTCACGTCTGCCAAAAGATAACACGAAGATGTTCAAAGCTAAGCCCCCAGGCTGGTAAGAGCTCCAAGGCACCAGCAGTGTGTGCAGA |
| | | ACTGGGGGGAGTCTGTTCTCCCAGGGATGCTCCCATCACCTGCTGCCAGCAGTGGGGCATGCCGGTCCCCTGGGGTGTGGCCAAGGGGC |
| | | TGTGTCTCCTGCCCGGGCTGCCGGCCCCTCTCAGGTTCACTTTCCCATCTCTAAGCCCACGTCTCGCTGCAGTTCAAGTTTGCCAGGCCAC |
| | | CAACGGGTGACACGCCCGGCGCAGTGGGGGACTCCGCACTTTCTGCGCAC |
| 132 | CELF2 | ACCCTTTGTGCCTGGGTCCCATAAACAATGTGCTTTTTAAAGGGGAGCCCCCTCCCAGCTCCGGCCTTTTTCTCCAGCGTGGGCAGCCAAT |
| | | CAGCTGCGCAGAGCTGCATAGCTGGACCGCTTTCCATTCTGAGTAGCAACAACGTACTAATTTGATGCACACATGGATGCCTCGCGCACTC |
| | | TGCAAATTCATCACCCGCATCTTGCATTAGTCATCTGACGGACTGCCAAGTGTTTCATTTTCTTTCCATGTGACTTTATTATTACCACCTC |
| | | TCTCCTCTCTTCCAAAAACCTCCCAAAAAGGGCGGTGGGGCGGGGGGCGGGGCAGGGAGAGGGAGAGAAATCCAGCAGACATCTAGCTCTG |
| | | CCTTTCTTTCCCAGCCACAGCCAGGGTAGGGCTGATAAGGCGCTGATGCGTTGATGGCAGCCTTGCAGAGCTAGACCTGCACTTAACTTGC |
| | | AGCTGCCTCCCGAGCCTCCAAGATGTCCACGCCCTGGGTGACAGGCGGCAGGGCGCTGCCCCGTGCTCCCCCGGCTCTGCTCGACAGCA |
| | | GCACGCAGTGAGAGCCTCGCCGCCGCCGAGGAGCAACTCATGGTGCCTCCGCTTTGTTTTAGTTCATCAAATTTCTACGACTCATTAGGCA |
| | | CTTTGCCACTGCTCTTCTTCCTCCTCCTTCCGCCTCCCCGCTCCCCCACCCCCACTATTTTTTCTTCCTGTCCCTCATCGTGCCGCCCTAA |
| | | CTCTGGCTCCCGGTTCCGTTTTTGACAGTAACGGCACAGCCAACAAGATGAACGGAGCTTTGGATCACTCAGACCAACCAGACCCAGATGC |
| | | CATTAAGATGTTTGTCGGACAGATCCCCCGGTCATGGTCGGAAAAGGAGCTGAAAGAACTTTTTGAGCCTTACGGAGCCGTCTACCAGATC |
| | | AACGTCCTCCGGGACCGGAGTCAGAACCCTCCGCAGAGTAAAGGTACAGAGCGCGGGGCGGGGGTCGCCAGGCGTCCAGGTGGGCGTCG |
| | | CGGGGCACTGGGGCTGTCCGAGCCCCCAGCCTGCAGGAGGAAGGGCGGGTAGGCAGGAGGGCTGGAAGCAGCCGGTGCTGGCGGCCC |
| | | CTGTGCTCCAGGGGCTGCTCCCGACTCCTCCCCGCACCCCCGCCCGCCTGCCCGCCGGGACAGGTTGGAGGCGGGAGAGAGGGACCGA |
| | | GGCAGGGCGGGAGCGCAGAGGCTCGGTC |
| 133 | HHEX | TAACAAATAAGCCGCCCGTGGTCCGCGCTGTGGGTGACCCTTGGCGCCTTCGAGGTCTGGAGCCCTAGGGTAAATAAGGAAACGGGGCGCC |
| | | TCTAGAGTTTTAAATGAACTCTGTTATTGGAAGCTTCAGTAGGGACCCTGAAAACAATTAACGTCTTAATTAGCATTTTAATGTCTCCATT |
| | | ATTACGGCGCGGGCTCTAGCTCAGCCCTTTACCTTACCTTCTCACCGTTAACAGGGGAGGGGGATTGTATTTTTAGTTCATCTTTTTATGT |
| | | TTTTGAGTTGTTATCCTGTCTGTCTGATTCCAGCCTCGAGGGTTTGATGATGCGGCCCGAGCCTGGCTGTGGTCGCCTGTCGGGGCTGGAG |
| | | CGGGACCCTCAGCCGGGCCGGGCCTGGGGGCTAACGTTTTCACAGTGCGCCCTGAGTTTCCTTGGGTTACTGCTGGGACCGCGCAGGAGGA |
| | | AGCAAAGAGTTTTTCGAGCTAGACCAACAGGAAACACATTGACGGAAATGTTGCCATAGCCCATGGGGTGGCTTTAACTGGCCGCCCCCGC |
| | | GGGCTGGGTGTGAAATCAGAGGAGGCCGCGGCTCCCCCGGCCAGGATTGGAGGCTCCTCGCGCAACCTAATGCGGGTGTCCGGGCCCG |
| | | AGCGCTTCCCGCGCAGCCAGGCCTTGTCGGTGCAGCAGCCCCGCTCCTCCCCAACACGCACACACCCGGTGTTCGCAAGTGCGGCTCAC |
| | | CAAGGGAGATCCAAGGGGGCAAAAAGTTATGTATAAATCCGAGAGCCACTGGGGAAAGAGGGTCGTGGTATTGTAAG |
| 134 | DOCK1/ | CTACCCTGTGCTATCCTGAGCTGTAGTCTTCTGAAATGATCGTTTGGCTTCCCAGCCAAGGCAGGGCTCCCCCAAAGTTCATTCCCACTCT |
| | FAM196A | TGCAGTTTCACCTCGGGATGCTTCCGCAGAATTTCAGCGCCTAAGCAGACAAGGTCAAAGTAAACCGCTTCACCGCTGCTTCTGGCGCAGG |
| | | GGCCCAGAGCGCGTGCAGCTCCCCAGCACAGACCAACAGCAGGAGAGGGGTCCGGGCGGGAGCCCTGGGCTGTAGATAAGCAAAACGCAC |
| | | CCATTTTCTCTCCTATTTACTCCAGAGGCACCTCTCCTCCCCCACTCCTGGCATCTCTTTATCACTGGCTCCCTCTCCCTGTGGCATATTT |
| | | TTGGGTAGTAGAATGCTGAGGTCACAGGGAGCGGCTCTTTATCCAAGCAGTGGGGACATCAGCCTGGAGCCCTGAGCATGAACCAGCAAG |
| | | ATGCAGACTCTCGCTCTTGACTTTGGGCTCCAGGAGCTGCCCCGACC |
| 135 | PAX6 | CAGTGCTCCGCTCCGGGAAATTGCATCGTCACGACAAACGGGACCGTGATAAAACGACCCTTTCCGTCCTTATTTGTAGATCACTCAGACG |
| | | AGATTGAACTGCACTTGTTTCCCCTTCGAGGGGAGCCGCGTTTTCAGGGTAGCCGAAGGCTTGGGGCTGAGGGGGGGCCCTCACCAAGG |
| | | CGCGGGTGGGGGCCGGAGCCTCAACTCGATGAGAAGTGACAGGCGTTTGGGGGATCTGGGCTCCGGCCGGGACCAGCGCAAGCAGGGACT |
| | | TTGCGGGGACACCGCTTCTCCAACAGAGCAAGGCCTGGCCCACGTTTCCGGTTTCTCCTAACTTCCTTTTATTGCCTTCCTTTGCTTCGC |
| | | AAGTTCCATCTACCCCTCCAGCTACAGAGCCCCACCTCTAGGCACAGGAAGCTTCCCGGAAAAAGAAAGGCTGTCCCAGAAAGAGACCGA |
| | | GAGAGACTTTCCAAACTTCGGGCATAGCCACGGCAATTCCCAGTCTGCTAATGCCAAGGCGGGCGCGTAAGGCCGCCTAAATCTAGACCT |
| | | CCCTCCTCACTCATTTCAAAAAATAACAACGTGCCAGCCACCTCCGCAGATACCGCCGGCTGGTGCTTGCCCAGGAGACGCCAGGGCCAG |
| | | AGCGCCACTCCCAGCATCGAAATGGCAGAGAGAAAGCGCAGCTCCAAATTCCCCTTCAGAGGTTAAGCCTCAATCATTGTGTCCCTTCCCT |
| | | AGGGACTGCTGGCGCTCTCGCCCACTGGCGATGATTATGCGCCTAGAACTCGACCGCGAAGCAACTAATAGGAAAACATATGGTGTCAATT |
| | | TGGATGCTCCGCGCCTCGCGCACACCCGGGAACGAGCGGCACAAAGCCCTGCCGGCCGGCCCGCGACCCCGCGCCCCTCGGGGCCTG |
| | | CCAGCCGGGCCGCAGCGACAAACGCTCAGGGCTGCGCGCCCTGGCTGGGGCCCGCCCGAGAGACAGCCTGCGGCTGGGGAGTCTGAG |
| | | CTCCAAGGGGAGAGCCCAGCCGCCGAAGGCGAGCCTACCGGCCAAGCCCTGGGGTCCGGCAGGTTCTGCACAACTACTCCCGCAAAGCT |
| | | CGCCACCTTTGTGCCCTTTCCTCAG |
| 136 | FERMT3 | GGGCCCTCGCGGCTCAAGCGCCAGCGCTGGAGAGAGAGTCTGAGGGTACCACGGGCGTGCTGGCCTGGGTGCTCACTCCCGCCCTCCT |
| | | TCATGAGCGGCTTTCCTCTGGGTGTGTCCAGGGCATCACAGAGCTCTTCTGCCCAAACCCGGAGGCCTACCAGGGCCTGCCCACCTTGCC |
| | | TCCTTCCACACTCTCTGTAGCAGCAGCCGCAGCCATGGCGGGGATGAAGACAGCCTCCGGGGACTACATCGACTCGTCATGGGAGCTGCG |
| | | GGTGTTTGTGGGAGAGGAGGACCCAGAGGCCGAGTCGGTCACCCTGCGGGTCACTGGGGAGTCGCACATCGGCGGGGTGCTCCTGAAG |
| | | ATTGTGGAGCAGATCAGTGAGTGTCCGCTGCCCGCTTGCTGAACTCGGCACCATGGGCGGCCGCCACGGGTGTCTCTGGGCACTTCCGG |
| | | GCCATCCCTGCTGCTCAGCTCCCGATAATGGTGTCACGGTGACTCAGGCATTAGC |
| 137 | PKNOX2 | TGTTTACGGAATCGGGATCGAGGGGCCGATAAGTAGTTTACACGCCGGCCAGAGCAGAGGGCTGGAGGTCGGAGTTGGGGGCTGGAGGA |
| | | ACGGGTGGCGTTTTTAGGATTCAGTAACAGGATCACAGCTTTTTCTTGTGGTGGAAGCTATTGGAATTTGGGGAGGGTAGCACGAGGGGTC |
| | | CTGCAGCTCCGCGTGTGAAAAAGCGTTTAGGTAGGCGATGAAAGTAGTTGATCTGAGCCATGGCAGGCGAGCCCCGAATTTTTGCTGCTTC |
| | | CCCCTGAAAGTGTTTCTTTAGGAGGAGAGGACTTGGGCCACACAGGACCCGGTCCTAAGAGAGCGATTCCGGGAAGCGGACAGATCGAAG |
| | | AGACCTTCTGGGCGAAGCGGCAGGGCAGCCTCGCGGGGCTGGGAGTGGATCTGAGGTCCCGACCCAGGCGGCTCGGAGTGCTCCAGGA |
| | | GCCACCTGGGTCTGCGGGCGCAGCGCGGCGGGGCGGGAGCGGTGGCCCGCAGGGGCCGCGGCCTGCGATGAAGGCCGGGGGGCAGC |
| | | GCTAGCAGCGAGGTGCCACAGTGGGCCGAGGAGTCTGGGCTGTGGCCCAGGGTAGGACCGGCTCA |
| 138 | KIRREL3 | ACCTAAACCAAGCTCTCCCTCCCTGCCGTCTCCTTCCCTGGCCTGGGTCTGAAGGAGAGGAGGTGCCCAGAAGTTCAGAGCGGCATAACC |
| | | ACAGAGATACTACCTAATTAACATACCAGAAGCATAAAGAACTCATTTGCATTGGAGAGT |
| 139 | BCAT1 | ATAACTACGGGGGTGGGGGTGGGGAAGGAAGAGATCCAAGGAGGCAGAAGGCTGCGGTCAAAATATTTTGGGGTGGCAGAGTCACGTAG |
| | | GATGTGGCTGTGGGTTCTGGCAGCCCAGAGATTCAGCTCCCGCCTCCTCCCTCAGAGCGAGTCCATAGCTACCCTCACGTCCCCCGTGGC |
| | | GGTCCTCGCCACGCTCCGGAGCGGGTTACCCATGAGGGTGCTAGACCTGGGCAGCGGGAACCTCGAAGAGGTGGAGATTGCAGGCTGG |
| | | GACTCCAGATTTCGGGCAGGGATGCGGGGAAGGGAAGACGCCTCGCTGGAGGCGGAATGGAGGGCAAGGCGAAGGAGGATGGTGCAGG |
| | | AAACGGCGACAAGGCGCCCGGCCAGGCCCGCGAGCTACCGAGACCCGGGTTCCAATCCTCCCCCCTTCCGCAAACGCCCGGGTTCGAG |
| | | GTACCTGGCGGGCAAGGGCCGCAGCGGAGCGAAGCGGGCTGGCCATGGGGAGGCTGCGGGGACGCGGGGCTGCAGAGAGCGGCAGT |
| | | GGCACGGAGCGCGCGGCTGGAAGCGAAAGCAGGCGGTGTGGCCAAGCCCCGGCGCACGGCCCATAGGGCGCTGGGTACCACGACCTG |
| | | GGGCCGCGCGCCAGGGCCAGGCGCAGGGTACGACGCAACCCCTCCAGCATCCCTTGGGGAGGAGCCTCCAACCGTCTCGTCCCAGTCT |
| | | GTCTGCAGTCGCTAAAACCGAAGCGGTTGTCCCTGTCACCGGGGTCGCTTGCGGAGGCCCGAGAATGCGCGCCACGAACGAGCGCCTTT |
| | | CCAAGCGCAGATATTTCGCGAGCATCCTTGTTTATTAAACAACCTCTAGGTGAATGGCCGGGAAGCGCCCCTCGGTCAAGGCTAAGGAAAC |
| | | CTCGGAGAAACTACAT |
| 140 | HOXC13 | CAGTCCAGCCGCTTGCCTCACTTCTTCCCGCTTGCCTTATCTCCCCGCAGACGTGGTTCCCCTGCAGCCCGAGGTGAGCAGCTACCGGCG |
| | | CGGGCGCAAGAAACGCGTGCCCTACACTAAGGTGCAGCTGAAGGAGCTAGAGAAGGAATACGCGGCTAGCAAGTTCATCACCAAAGAGAA |
| | | GCGCCGGCGCATCTCCGCCACCACGAACCTCTCTGAGCGCCAGGTAACCATCTGGTTCCAGAACCGGCGGGTCAAAGAGAAGAAGGTGG |
| | | TCAGCAAATCGAAAGCGCCTCATCTCCACTCCACCTGACCACCCACCCGCTGCTTGCCCCATCTATTTATGTCTCCGCTTTGTACCATAAC |
| | | CGAACCCACGGAAAGACGCTGCGCGGGTGCAGAAGAGTATTTAATGTTAAGGAAAGAGAAGAACCGCGCCGCCCGGAGGCAGAGAGGCTC |
| | | CATGGCCGTGCTGCTGGGCCATCCCCAACTCCCTATCCCATCCCCAGCCTCCACCCCCATCCAGATGGGACTCACGTGGCTTCAACAGCT |
| | | TTGGAAATGGGTCCCGAGTGGGCCGTGCGAGGAAGGCTGTCGACCTCTACTCCTCCTTGC |
| 141 | TBX5 | CAAGATCGACTTTCTTAGGAAGGGGGAGAGGAGGGAACTCTTCACGAAGGGAGGTGGGAGTCCACCTCAGACCTCTATTGGAAGGAAATCG |
| | | AGTTGTTCCGGGGGACTGAGGTCTCTTGCATAAGGCATGGGATCCTTATTATTATTATTATTATTTTTAAATCCCCCGCGGAGGAGCTCTG |
| | | GGCAAATGAATACCGAGGCGCCGCTCTAGCTGGTTAGGCTTGGGATGCGATAACTCAGTGCCCTCTTGCAGACTTGCATAGAAATAATTAC |
| | | TGGGTTGTCGTGGAGGGGACACGAGACAGAGGGAGTTCTCCGTAATGTGCCTTGCGGAGAGAAAGGTCCAAGAATGCAATTCGTCCCAGA |
| | | GTGGCCCGGCAGGGGCGGGGTGCGAGTGGGTGGTGGAGTAGGGGTGGGAGTGGAGAGAGGTGGTTTCTGTAGAGAATAATTATTGTACCA |
| | | GGGCCCGCCGAGGCACGAGGCACTCTATTTTGTTTTGTAATCACGACGACTATTATTTTTAGTCTGATCAATGGGCACAATTTCTAAGCAG |
| | | CGCAGTGGTGGATGCTCGCAAACTTTTGCGCACCGCTGGAAACCCACTAGGTTGAGTTGCAAAACGTACCGCGTAGACGCCCCTGGTGGC |
| | | GCCGAGAGAAGAGCTAGGCCTGCCCAGCACAGAGCCGGAGAGCGTCGGGCCTTCCGGAAGGGTAAGTTCTCCGCCAAGGGGTCCCGAG |
| | | GGAGCTGGACGTCTGAATCTGGACTTGCCCCCAGCTTCGGGGTTCGATTCTGGGTTTTGCGCGTCCCCAACCCCCAGGGCTTTCCGAAGC |
| | | ATGGCCTGGCTCCAGGCCCGGTCCTGTAAGGACTGGAACGGCAGCAAAATGTGCAGGGAGGCAGTCGGCCGGCAGAGCTGCGGCGGGA |
| | | GCCAAGGTCAGGCCCGCGGGGAGAGCGGGCAGCTTCCAGCGCCGGCCACAAGCTCCCAGGCCAGCTGGGCCGCAGACCCCTTTGCTTC |
| | | CAGAGAGCACAACCCGCGTCCTTTCTCTCAGCCAGGCTGCAGTGGCTGCCCCGAGCTTCGCTTTCGTTTCCCAAGCTGTTAATAACGATAT |
| | | GTCCCCAAATCCGAGGCTCGTGTTTGCTCCCAGATGCCAAGAACGCAACCCGAAATCCTTCTCCCAAACCCTAGGTCGACGAGATGAGTTC |
| | | CTACTTGACCTCTGAGCCGAGGTGGGCCGGAAACCGAGGCCTAGGCCCCGCCGGGGCTGCAAGGAAAAGGGGAAACTCCGAGCGTAGC |
| | | GTCTTTTCCTTGTGGTTCCTTTCTCCGGCATCCCGGACTGCGGGCCCTGCAGCCACCTGGACCGGCATTCAAAGGATTCTGCAAGTCCAGC |
| | | TTCACAGACTGGCTTTCCCAGACGCTCCGAAGCCCGCACCACGAACAGAATAAAGGAGAGACGAGAGATCGCAACTAGATTTGAGAATCCT |
| | | CGTTCTTTTCCCCAATCGTTCGGGCAGTAAACTCCGGAGCCGGCTACAGCGCGCATCCTC |
| 142 | TBX3 | ACTGTCCTCCTCCCTCAATTGCCTATTTTTTGCCCATAGCTCTAACTTAACCCTGTGATCACCCCAGATCGCTACTTCTGACCCCCATCTC |
| | | CTCTCCCACACCAACCTCCAGCGCGCGAAGCAGAGAACGAGAGGAAAGTTTGCGGGGTTCGAATCGAAAATGTCGACATCTTGCTAATGGT |
| | | CTGCAAACTTCCGCCAATTATGACTGACCTCCCAGACTCGGCCCCAGGAGGCTCGTATTAGGCAGGGAGGCCGCCGTAATTCTGGGATCAA |
| | | AAGCGGGAAGGTGCGAACTCCTCTTTGTCTCTGCGTGCCCGGCGCGCCCCCCTCCCGGTGGGTGATAAACCCACTCTGGCGCCGGCCATG |
| | | CGCTGGGTGATTAATTTGCGAACAAACAAAAGCGGCCTGGTGGCCACTGCATTCGGGTTAAACATTGGCCAGCGTGTTCCGAAGGCTTGTG |
| | | CTGGGCCTGGCCTCCAGGAGAACCCACGAGGCCAGCGCTCCCCGGA |
| 143 | chr12: | CTCAGGGAATCACATGTCCGCCTGGCCTGGCCTGGTACCAAATGTTTATAGACAGGACGAGGGTCGCTGGAATCGCCTCGCTCCTTTCAGC |
| | 113622100- | TTGGCGCTAAGGCGCGAATCTCGATCCTCCTAGTATTTCTCTGGCGTCTGTCTCTATCTCAGTCTCTGCTTTTGTCTCTTTCTCCCTCCCT |
| | 113623000 | CCGCCCCAGTCTTTCCGTCTCTTTTTCCTCGAATGCACGTGGAATTCGGAATTGAAAATTGAGGTCAGAATCTCCCTTTTTCTTCCAGTTA |
| | | TCCGCGCCGCTGCCCCACGCCTAGCGGCTTGGATCTGCATAGACATCTATCTACCCGCAACAAGATCCGAGCTGCAGAAGCAAACCTAATC |
| | | TGTCTCCGCACCATCCCCTGCTCTGTAGACCCACTGCCCCATCCCACGCCACATCCTTGAGGTTCAAGTAGCGACTCCAGCGGATGATTCG |
| | | GAGAATGCCCTGCTTTCCAAAGGCCCCAACCCGTGTTTTTATTTTCTTTTTCCTTTGCCCGCTTGACCAACTTTGGTTTCTTTCAGGGCCC |
| | | GGAGGTGCCTGCGCCGCGCTTGGCTTTGCTTTCCGCCGCCCCAGGAGACCCGGGACTGTGGTTTCCGCTCGCCACATCCCAGCCTGGTGCG |
| | | CACACAAGAGCCTGGCGAGCTTCCCTCGCGCGCTTACAGTCAACTACTTTGGGCCTCGGTTTCCCTGCTCCTTGTAGATCAGAGAAGGGAC |
| | | GGGCGAAATGCCTGCGAGGGAGGGTTGGCGAATGGGTTGGTTGGTGGCAAGACTGCAGTTCTTGTACATGGACGGGGGTTGGGGGGTCAA |
| | | CACTGGAAGAACTCCTGCCTGACGCCAAGAGCCACCCGCTTTCCAGCTCGTCCCACTCCGCGGATGTTTACCCACCTTCATG |
| 144 | chr12: | TTTGGGGCACCCAACCCTTCCCAAGCCTCGGTTTTCCCGATCTTGTGGGATCCTTGCGGCGCGAATGGGGTTGGAAGCACCTTGGAAGCT |
| | 113657800- | ACAGAGTACCGGGTCGGGACAATTTCCGGCACTGCCCCAGTTCAGTGGTTTATAGAAAATTTCTTTCTCTCTCTCAGGTCCACTAAGACC |
| | 113658300 | GAGAGAGAGAGAGAAGTCGACTCTGGCACACCCGGGCGAGGGGCTGCCGGGATTCGGGAGCTGGCGCGGTTGATTTTTTCCGAGAATCCT |
| | | CCACTTGGGGTGACGTCGGGCAGCGCGCGCGGGCCGTGAGGTTAATGCCCAGGCTTTTCTCTAAAGCGTCCGGGAATGATCCGGCGAATA |
| | | AAACGGGTGTCTGCAAAGTTAATGAATTGTACAAGGAGGCTGAGGGTGGGGACTTCGACCCGGGGAGCCAGAGGCGGTTCTGGTGGACG |
| | | CTTCCCCGTGCGCCTAGGGGTGCGCTGGGCTTTCCCAGCCGAGGTCTGCAG |
| 145 | THEM233 | CCAGACAGTTAAGGTAAAACGTTGAAGTCAAGAGGAAGTAGTGAGTCTGTTGCCAACTGGATAGGGTTGGTCCTGTCCCATCTAAATGTAT |
| | | TAGAATTAAGTGGCTTTTAAAAATGAGCTGGTCATCTTCAGCCCACGGGCTGGCCAATTTGGAACTTAATGGGCCTTTGCGTCCTCCTTCC |
| | | CTGAGCCTCCTTTTATTCCAGACTTCTCAGTGTGAGTCTGTGCGTCCCTCCGACGATCTCAGGGAGTGGGGTGCCTTCATCTGCCTGTTCC |
| | | CTGTTCCTCAGGCTGACGCTCCCGCTGTCCTCCCCGCCTCCCCTCACTCCTTTTCTCCCTCCCTTCCTCCTTGTGGGGAGGCTCTTGGCCA |
| | | GGGTCCCTGAGCCCGGGCGGGTGCTGGCAGAGGACGCAGAAGGGGTGAGGTCACGTCTCCCTTGAGCCCCGAGCCGCTGGCTTTTCAGAG |
| | | CCTCGCCACAAGCCGGCGGCCAGAGCCCCAGACCACACAGACCGTGCGCTCCTCCGCCCTCCCGGCGCCGCCGGCCTCGCCCATGTCT |
| | | CAGTACGCCCCTAGCCCGGACTTCAAGAGGGCTTTGGACAGCAGTCCCGAGGCCAACACTGAAGATGACAAGACCGAGGAGGACGTGCC |
| | | CATGCCCAAGAACTACCTGTGGCTCACCATCGTCTCGTGTTTTTGCCCTGCGTACCCCATCAACATCGTGGCTTTGGTCTTTTCCATCATG |
| | | GTGAGTGAATCACGGCCAGAGGCAGCCTGGGAGGAGAGACCCGGGCGGCTTTGAGCCCCTGCAGGGGAGTCCGCGCGCTCTCTGCGGCT |
| | | CCCTTCCTCACGGCCCGGCCCGCGCTAGGTGTTCTTTGTCCTCGCACCTCCTCCTCACCTTTCTCGGGCTCTCAGAGCTCTCCCCGCAATC |
| | | ATCAGCACCTCCTCTGCACTCCTCGTGGTACTCAGAGCCCTGATCAAGCTTCCCCCAGGCTAGCTTTCCTCTTCTTTCCAGCTCCCAGGGT |
| | | GCGTTTCCTCTCCAACCCGGGGAAGTTCTTCCGTGGACTTTGCTGACTCCTCTGACCTTCCTAGGCACTTGCCCGGGGCTTCTCAACCCTC |
| | | TTTTCTAGAGCCCCAGTGCGCGCCACCCTAGCGAGCGCAGTAAGCTCATACCCCGAGCATGCAGGCTCTACGTTCCTTTCCCTGCCGCTC |
| | | CGGGGGCTCCTGCTCTCCAGCGCCCAGGACTGTCTCTATCTCAGCCTGTGCTCCCTTCTCTCTTTGCTGCGCCCAAGGGCACCGCTTCCG |
| | | CCACTCTCCGGGGGGTCCCCAGGCGATTCCTGATGCCCCCTCCTTGATCCCGTTTCCGCGCTTTGGCACGGCACGCTCTGTCCAGGCAAC |
| | | AGTTTCCTCTCGCTTCTTCCTACACCCAACTTCCTCTCCTTGCCTCCCTCCGGCGCCCCCTTTTTAACGCGCCCGAGGCTGGCTCACACCC |
| | | ACTACCTCTTTAGGCCTTTCTTAGGCTCCCCGTGTGCCCCCCTCACCAGCAAAGTGGGTGCGCCTCTCTTACTCTTTCTACCCAGCGCGTC |
| | | GTAGTTCCTCCCCGTTTGCTGCGCACTGGCCCTAACCTCTCTTCTCTTGGTGTCCCCCAGAGCTCCCAGGCGCCCCTCCACCGCTCTGTCC |
| | | TGCGCCCGGGGCTCTCCCGGGAATGAACTAGGGGATTCCACGCAACGTGCGGCTCCGCCCGCCCTCTGCGCTCAGACCTCCCGAGCTGC |
| | | CCGCCTCTCTAGGAGTGGCCGCTGGGGCCTCTAGTCCGCCCTTCCGGAGCTCAGCTCCCTAGCCCTCTTCAACCCTGGTAGGAACACCCG |
| | | AGCGAACCCCACCAGGAGGGCGACGAGCGCCTGCTAGGCCCTCGCCTTATTGACTGCAGCAGCTGGCCCGGGGGTGGCGGCGGGGTGA |
| | | GGTTCGTACCGGCACTGTCCCGGGACAACCCTTGCAGTTGCGCTCCCTCCCCCACCGGCTCACCTCGCCTGCAGCTGGGCCACGGAACT |
| | | CCCCGGCCACAGACGCA |
| 146 | NCOR2 | CTCTCTGGGCCTTAGGAAAATGGAAATGACACCTGTACCTGCCCTTCCAGGACTGACAGGAGGGGCTGCTCCATGAAACCTCACTGCTGCG |
| | | GTCATAATGTCATTATCTTTTGCCTTAAAGGGATTTCTTCTGCACCAGCACCTAAAGTGGCAGCCCCTTACCCTTGGCCATCAGCTGGACC |
| | | CTGGTGCTCTCCTGGAGCCCAAAACCTCTGTTTTGTGTTGCATCCTGCTGACCAGCCACAGTCCACACCCATCTGAGTGTCTGAGCAGAAC |
| | | AGCCCAGAGGCCACACCAGGATGGCTTTCCACCGGTCACCTTCCCCCACCCACTCATAAACCCTGCGTCTCTGGGGGAGAGGGTGGCGA |
| | | GGTCCCCTCCCCACATAGATGGAAACACTGAGGCCTGATTCATGGTGCCCCCTGTGAAGCGCCTCATGGCCAGCACCGGGGGGCAGCAG |
| | | GCCAGGGCGGGGACACATACCCGGTTCTCGTCGTAGATGATCTGCACCAGGCTGCGGTGCTTCGACTCGATGGGCGGCGGTGACACGGG |
| | | CTTCTCAGGCTCGGGCGGCTTGGCAGCCTCCTCCTCCAGCTGTTGCTGTGGGGAGAGGCA |
| 147 | THEM132C | CTTGAAAACTCCCAGCCCCCTTTGTCCAGATGGGGATGGAGGTGGCCAGGCTGCCCCGTTGATTGTGTGCCGAGGAGCCCTCCCCGGGAA |
| | | GGCTGTGATTTATACGCGCAGGCTTGTCACGGGGTGAAAGGAAGGGCCACTTTTTCATTTTGATCCAATGTTAGGTTTGAAAGCCACCCAC |
| | | TGCTGTAAACTCAGCTGGATCCGCGGGCCGTGATTAAACACATTGCCCGCTTTGTTGCCGAGATGGTGTTTCGGAAGGCGCTGTGAATGCA |
| | | CTTCCCTTTGCGGGGCTCACACAGACAAGATGTGTGTTGCAAGGATGAGGCGCCTGCTCGGCCTCCAGCCCAGGGCCGGGAAGGGAGAA |
| | | GGTGCTGTGCGTCGCTGCCTGTGTCGCCCGCGGCTCTCC |
| 148 | PTGDR | CGCGTCAGGGCCGAGCTCTTCACTGGCCTGCTCCGCGCTCTTCAATGCCAGCGCCAGGCGCTCACCCTGCAGAGCGTCCCGCCTCTCAA |
| | | AGAGGGGTGTGACCCGCGAGTTTAGATAGGAGGTTCCTGCCGTGGGGAACACCCCGCCGCCCTCGGAGCTTTTTCTGTGGCGCAGCTTCT |
| | | CCGCCCGAGCCGCGCGCGGAGCTGCCGGGGGCTCCTTAGCACCCGGGCGCCGGGGCCCTCGCCCTTCCGCAGCCTTCACTCCAGCCCT |
| | | CTGCTCCCGCACGCCATGAAGTCGCCGTTCTACCGCTGCCAGAACACCACCTCTGTGGAAAAAGGCAACTCGGCGGTGATGGGCGGGGT |
| | | GCTCTTCAGCACCGGCCTCCTGGGCAACCTGCTGGCCCTGGGGCTGCTGGCGCGCTCGGGGCTGGGGTGGTGCTCGCGGCGTCCACTG |
| | | CGCCCGCTGCCCTCGGTCTTCTACATGCTGGTGTGTGGCCTGACGGTCACCGACTTGCTGGGCAAGTGCCTCCTAAGCCCGGTGGTGCTG |
| | | GCTGCCTACGCTCAGAACCGGAGTCTGCGGGTGCTTGCGCCCGCATTGGACAACTCGTTGTGCCAAGCCTTCGCCTTCTTCATGTCCTTCT |
| | | TTGGGCTCTCCTCGACACTGCAACTCCTGGCCATGGCACTGGAGTGCTGGCTCTCCCTAGGGCACCCTTTCTTCTACCGACGGCACATCAC |
| | | CCTGCGCCTGGGCGCACTGGTGGCCCCGGTGGTGAGCGCCTTCTCCCTGGCTTTCTGCGCGCTACCTTTCATGGGCTTCGGGAAGTTCGT |
| | | GCAGTACTGCCCCGGCACCTGGTGCTTTATCCAGATGGTCCACGAGGAGGGCTCGCTGTCGGTGCTGGGGTACTCTGTGCTCTACTCCAG |
| | | CCTCATGGCGCTGCTGGTCCTCGCCACCGTGCTGTGCAACCTCGGCGCCATGCGCAACCTCTATGCGATGCACCGGCGGCTGCAGCGGC |
| | | ACCCGCGCTCCTGCACCAGGGACTGTGCCGAGCCGCGCGCGGACGGGAGGGAAGCGTCCCCTCAGCCCCTGGAGGAGCTGGATCACCT |
| | | CCTGCTGCTGGCGCTGATGACCGTGCTCTTCACTATGTGTTCTCTGCCCGTAATTGTGAGTCCCCGGGCCCCGAGGCAGCAGGGCACTGA |
| | | GACTGTCCGGCCGCGGATGCGGGGCGGGAAGGGTGGA |
| 149 | ISL2 | CTTCCGCCGCGGTATCTGCGTGCCCTTTTCTGGGCGAGCCCTGGGAGATCCAGGGAGAACTGGGCGCTCCAGATGGTGTATGTCTGTACC |
| | | TTCACAGCAAGGCTTCCCTTGGATTTGAGGCTTCCTATTTTGTCTGGGATCGGGGTTTCTCCTTGTCCCAGTGGCAGCCCCGCGTTGCGGG |
| | | TTCCGGGCGCTGCGCGGAGCCCAAGGCTGCATGGCAGTGTGCAGCGCCCGCCAGTCGGGCTGGTGGGTTGTGCACTCCGTCGGCAGCT |
| | | GCAGAAAGGTGGGAGTGCAGGTCTTGCCTTTCCTCACCGGGCGGTTGGCTTCCAGCACCGAGGCTGACCTATCGTGGCAAGTTTGCGGCC |
| | | CCCGCAGATCCCCAGTGGAGAAAGAGGGCTCTTCCGATGCGATCGAGTGTGCGCCTCCCCGCAAAGCAATGCAGACCCTAAATCACTCAA |
| | | GGCCTGGAGCTCCAGTCTCAAAGGTGGCAGAAAAGGCCAGACCTAACTCGAGCACCTACTGCCTTCTGCTTGCCCCGCAGAGCCTTCAGG |
| | | GACTGACTGGGACGCCCCTGGTGGCGGGCAGTCCCATCCGCCATGAGAACGCCGTGCAGGGCAGCGCAGTGGAGGTGCAGACGTACCA |
| | | GCCGCCGTGGAAGGCGCTCAGCGAGTTTGCCCTCCAGAGCGACCTGGACCAACCCGCCTTCCAACAGCTGGTGAGGCCCTGCCCTACCC |
| | | GCCCCGACCTCGGGACTCTGCGGGTTGGGGATTTAGCCACTTAGCCTGGCAGAGAGGGGAGGGGGTGGCCTTGGGCTGAGGGGCTGGG |
| | | TACAGCCCTAGGCGGTGGGGGAGGGGGAACAGTGGCGGGCTCTGAAACCTCACCTCGGCCCATTACGCGCCCTAAACCAGGTCTCCCTG |
| | | GATTAAAGTGCTCACAAGAGAGGTCGCAGGATTAACCAACCCGCTCCCCCGCCCTAATCCCCCCCTCGTGCGCCTGGGGACCTGGCCTCC |
| | | TTCTCCGCAGGGCTTGCTCTCAGCTGGCGGCCGGTCCCCAAGGGACACTTTCCGACTCGGAGCACGCGGCCCTGGAGCACCAGCTCGCG |
| | | TGCCTCTTCACCTGCCTCTTCCCGGTGTTTCCGCCGCCCCAGGTCTCCTTCTCCGAGTCCGGCTCCCTAGGCAACTCCTCCGGCAGCGAC |
| | | GTGACCTCCCTGTCCTCGCAGCTCCCGGACACCCCCAACAGTATGGTGCCGAGTCCCGTGGAGACGTGAGGGGGACCCCTCCCTGCCAGCC |
| | | CGCGGACCTCGCATGCTCCCTGCATGAGACTCACCCATGCTCAGGCCATTCCAGTTCCGAAAGCTCTCTCGCCTTCGTAATTATTCTATT |
| | | GTTATTTATGAGAGAGTACCGAGAGACACGGTCTGGACAGCCCAAGGCGCCAGGATGCAACCTGCTTTCACCAGACTGCAGACCCCTGCT |
| | | CCGAGGACTCTTAGTTTTTCAAAACCAGAATCTGGGACTTACCAGGGTTAGCTCTGCCCTCTCCTCTCCTCTCTACGTGGCCGCCGCTCTG |
| | | TCTCTCCACGCCCCACCTGTGTCCCCATCTCGGCCGGCCCGGAGCTCGCCCACGCGGACCCCCGCCCTGCCCCAGCTCAGCGCTCCCTGG |
| | | CGGCTTCGCCCGGGCTCCTAGCGGGGAAAAGGAAGGGGATAACTCAGAGGAACAGACACTCAAACTCCCAAAGCGCATGATTGCTGGGAA |
| | | ACAGTAGAAACCAGACTTGCCTTGAAAGTGTTTAAGTTATTCGACGGAGGACAGAGTATGTGAGCCTTTGCCGAACAAACAAACGTAAGTT |
| | | ATTGTTATTTATTGTGAGAACAGCCAGTTCATAGTGGGACTTGTATTTTGATCTTAATAAAAAATAATAACCCGGGGCGACGCCACTCCTC |
| | | TGTGCTGTTGGCGCGGCGGGAGGGCCGGCGGAGGCCAGTTCAGGGGTCAGGCTGGCGTCGGCTGCCGGGGCTCCGCGTGCTGCGGGCGG |
| | | GGCGGGCCCGGTGGGGATTGGGCGC |
| 150 | chr15: | AGTTTGGGGAGCCTTTTCTCCATTTGAGAAAAAACAAACTTACAGCGAGGGGTGAGGGGTTAGGGTTTGGGATTGGGGAAAATGTGGGTGG |
| | 87750000- | GGAGCCCCCCCAAGGAAGTGAGGAGGGGGCTGCAAGGATTACACCTGGGCATACGTTTCCCTAGAAATCACATTCATTGTATTTTTATAAT |
| | 87751000 | TTATTCTAAATCTTTCATGCGAAGAAAGTCAGTAGTGAGTGTTAGTACTGGTGGCCCTCCTGATCACACTTGCATCTCTTGAGTGTGCCTT |
| | | AAAGGTCTTGGGAATGGAAAATATAAAAACTGCTTCGTGATGCGTCATCTTTATCCCCCACTCCCCCACCCATTCCAATATATTTTCTACT |
| | | TCCAGCCTAAATTCGGGGCCCCCTACCGAGGCCGGCCATGATCTTGAGGGCGGCATAGGGGAGGCCGCGCTCTGTCCACCCCAGCCTGGTG |
| | | ATGCCGTTCGCTTCTTGTGCCCGGTATTGTGGGCTACATGCCTTTCCGGCGTACGGAGCTGAGCGTCCAGGCCAGTGCCCCTCAACCTCTC |
| | | AGTAATGTTTACCCGAGGCCGTCGTGCAATGAGACTATTCGCATGGCATTGTCAACGCGGCGGCGCGCGCGTCTCGGCCCTCCGCGGCTTG |
| | | CCAGACTGTCCTGCAAACCACCTCACCCGTCTCTTTGGCGCAGGAGACTCAGGCTGTAACCGGAGAAAACACTTCACCCTGGAACCCTAAC |
| | | TCAGGTCCTGGCAAAAGATGCGAGAGGAAGACTTGCTCTCTTAATAAATCTCGGCCGCCCGCACATCTGGCCCCTAGACCTGCTCGGTAGA |
| | | GGACTGGCTGGTGGATGCGCGGTCCAGGCCGTGGGCACTCGACCCACCTCTATTTTCCTTCCCGAGGCGCCCCTGGATTACCACTTTCGG |
| | | TTTGCGCTTACATCCGGGATGTCGAATTTCCCAGGGAATCATAATTATTTTATCTATAATTTATTCTAACCCCAAGGTTCCAAGAAAATCT |
| 151 | chr15: | ACATTCCTTCTAAAATGTGGGCTTTCTGTGTACATGGGCGCGCATTCCCAGGACTCGGTTCCCTGGGTGGAATTCACCCAGGAATACAATC |
| | 87753000- | GATTTTCTGAACCTGCGTAAGGCCACAGGCAGCTCTGAAAATGAAAGCGTTTGCTAAGTGGGGGAGATCTCACCGATCGAACGTTTAAAAA |
| | 87754100 | TGGCTTTGTCTTCATTCAGCTCTCCCGATTTATTCTGTGTTTTACAAATAGAAGCTCAGAGCTTCTGTCGCCCAGTCCTTGCATGACTCAT |
| | | GGCGGTGGCCACACGGGTTTCAGGGATAACGGGATGTTTAGAAAATCGCTGCATATCGGAGTTTCCTAGCACGTTCCATTTATACTGAACG |
| | | CAGGCGGCCGCTGAAAATCCAGCCTCGACTCTTGCTAATGACTGGGTAGGACCCTCGGGGTCCTGCGACGGTGCTGGAGGGTGTTCCCGGC |
| | | TCCGATGTGGGGAGGCCTGCGCGGGGACTAGGTTCTCGAGAGGCGAGCGGGCGCGCCAGAGAACCCGAGACTGCTGCGGGGCCGGAT |
| | | GCGGGATCCCTGGGCTGCGGTTCTACGCAGAAACGCCAATGGCCATGCCTCCCCAGCTCCTCCCAGCCCCAGTCACTAGGCCGGCGCCT |
| | | GGCCCGGAGATCCTCCCAGAGCCCTGGCGGTGCCATCATGCCGGAGAAGACAAGCTCGGCCCCGCTGGAATTCGCTCCAAACACAGATGCT |
| | | CATTTTTGGAATATTCTAGAAAAATAACAAGATCTTGTTTGTCGTTATGATTCACGGGAGGTAACTGATGGGAGGGCCATTTACATGAGGG |
| | | CAGACACTGTGGGGCGAAGGTGACTTCTGGACGTAGGCTTTAAAGTAGGAACGGCTCCAAATTCCCAATATCTCCGGCCTTACCGGTTGCA |
| | | AATCGGACCCCTGCGGGAAAACCAGACACTTCTGTTTCGTGGCTTTCGGGCTGCCTCCAGCCCACGCAGGCTCGTTTAGTCCCCGTGGAG |
| | | TCAGCCCCGAGCCTTCCTAGTCCTGGAACAAGGGCTCCAGGTCGCGGCCGCGGGAAGCCGCCAAGAGGGCGGGGAGTAGGGATTCCCT |
| | | CCAGCTCCGCAGGGCATC |
| 152 | NR2F2 | TCCTCCTCGGCCTCAGATGTCGTCCCACCTGCCCACGAGCAGGGAACCTGGAACCCACTCTCCCGGCAGTCCCCAGCGGGTTCCGCCAC |
| | | CCGGCGGCCGCCCCTGACACCGAGTGGGTGGGAGGAAGAGGCAGCTGGCGGGGATGGGCCATTGAGACCTCTTGAAAAATATTAAAAGA |
| | | CAGGATGGGTAGAGATTTCTCCGGGAGAAAGTTCGAGGGTGCATCGGGTCGCGGCTGGGAGGAGTACCCGAAATGCCAGCAGGAGAAAT |
| | | GCAACCTGTTTAGGCCACACCTTCAATCCCCGAGGCTGTCTGGAGAGACTGCGTGCGGGGGACTTGCCGGCGTTCCCACACCGCGCCTG |
| | | CAATCCACTCCCGCGGCTGCCTGGCCTCTGCCACTCGCGGCTTGAAGCCAGTGGCTCTCAAGCCCTCGGCCCCGCGGCGGCCCGCGCAG |
| | | CCTTCACCCGGCGCCGGCACCACGAAGCCTGGCCGCAGTGGACTCCCCGCAGCTCGCTGCGCCCTGGCGTCTCCCGTCGAGGAGGGAG |
| | | GGACGGAGGCCTGAGCCGGGAGCTCCCTGGCGGTGGTCGGGCCGCCCCCCTTGAGGCCTGCTCCCCCCTCTCGGCCTCGCCAAATCCCTG |
| | | AAAGCCCAGTCCCCCTTCGTCACCCCGGGGGCTTCTAATCACTCGGTATCGATTTCCCTAACTCTTTTCATCCTGTTGAAGACACATCTT |
| | | AAAACACTCCAGCCCGGAGTGTGCTCTGGGCTTTATCCACACTAATAAAATGATTTACCCTTCTCTCCGCGCTCTCCTCACAGAGGAAAA |
| | | TCGTTCGAGCCCCGGCTATTTGTGTGTGATCAGTAAATATTTAGTGCGCTGACATCCTTAGCTGGGCTTCGGATCGATTCGGGGCCCACCG |
| | | GGAGGTGCGCACGGTCCGGGCGGGGCCGCGCCGAGCTCGCCGAGGGGGCTCCTCCCGCCCTCGCCGCCGGCCGCTGATTTACGGCCCCTG |
| | | CAACCAGCTAAGGGGGGCGAAAGCGCGCCTGGAAAATTGGCTTTTCAACCTTTTACTTTTGACATTCAGCCACTTCCCCAGGCTCTAATTC |
| | | TCGCCCGCACTCCTCCCTCCCGCCCTACTAAGGGTTGCCCTGTGCGCCCTGCGAGCCCTTCCAGCAGCAACGCGCGGCGCTCGCGCCCC |
| | | CTCGGCCCGGGGACCACCTATCACAGCCCTGAGCCGCGACGCGGGGAGGCCCCGGCCCCTGCTATGGGGGTCGCCTCCTTCGAGGAGA |
| | | GATGCTCTCCGCCCGCCCACACCTCTGAGGGAGGAGAGGGGGTGGAGAAGCCCAGAGCTGCATCTGCTGGATGACGAGCCGCTCTCCCT |
| | | GCTACCCTTTCTCCGACCCGTCGGCCTTTCTCCTACTCTGGAGACTGATCCTCGACGTCCATCGGGCCGGATGGCGTCGGGTGGAAGCGT |
| | | TACTTTCCTCGCAGAAAAACTCCTCCTCTTTCCTAAGATCAGAAAAAGCGCTTAGCTTGGAATTGTTAG |
| 153 | chr16: | CCTAGGCATTCTCAGCCCGTTTTGCTGGAGGGGGCATTTGAGGCCTGGCCAGCTTAGCCAGCCTACAAGGAGTGTTACTGGGGTGAAAAC |
| | 11234300- | AGCCAGCGGGGACCAGTCTGCTTGTGGCCCGCCAGGTGCCTGGGATGGGGAAGCAGCAAATGCCCACCTTCCTGCCCAACCCCCTCCTC |
| | 11234900 | CCTCTTCATGGGGGGAACTGGGGGTGGCAGCGGCTGCCGGGTGCGAGCGGGCTCAGGCCTGTGGCCCTGCCTGACGTTGGTCCCCATC |
| | | AAGCCATGTGACGAGACCAGGCCACAAGAAAGAGGTTTCAACAAGCGTTATCGTTTCCTGGAACTCCAACTCGGCGACTTCCCCGAAGAC |
| | | CGGCTGTGCCTGGCGGGCGGGCTGCGCACAGCGGGGACAAGGCTGCCCCCTTCCTCCTCCGCTGCCTCCGCGGCCGCGTCTATCTCAGT |
| | | CTGACTACCTGGAAGCAGCACTCCACCCTCCAGCCCAGCGGCCCTCGGCTCAGCTGCCAGGTCACCGGCAACCCCGGGAGCGGTGGGG |
| | | CAGGGGCTGCTCCGCCAGCCTCTGTGATGTTCAGGCCGGGCTGCACCAGCCCGGGACCCCTAGGTG |
| 154 | SPN | GCACTGGTTCCCCTTTACCTGAGCCAACAACCTACCAGGAAGTTTCCATCAAGATGTCATCAGTGCCCCAGGAAACCCCTCATGCAACCAG |
| | | TCATCCTGCTGTTCCCATAACAGCAAACTCTCTAGGATCCCACACCGTGACAGGTGGAACCATAACAACGAACTCTCCAGAAACCTCCAGT |
| | | AGGACCAGTGGAGCCCCTGTTACCACGGCAGCTAGCTCTCTGGAGACCTCCAGAGGCACCTCTGGACCCCCTCTTACCATGGCAACTGTCT |
| | | CTCTGGAGACTTCCAAAGGCACCTCTGGACCCCCTGTTACCATGGCAACTGACTCTCTGGAGACCTCCACTGGGACCACTGGACCCCCTGT |
| | | TACCATGACAACTGGCTCTCTGGAGCCCTCCAGCGGGGCCAGTGGACCCCAGGTCTCTAGCGTAAAACTATCTACAATGATGTCTCCAACG |
| | | ACCTCCACCAACGCAAGCACTGTGCCCTTCCGGAACCCAGATGAGAACTCACGAGGCATGCTGCCAGTGGCTGTGCTTGTGGCCCTGCTG |
| | | GCGGTCATAGTCCTCGTGGCTCTGCTCCTGCTGTGGCGCCGGCGGCAGAAGCGGCGGACTGGGGCCCTCGTGCTGAGCAGAGGCGGCA |
| | | AGCGTAACGGGGTGGTGGACGCCTGGGCTGGGCCAGCCCAGGTCCCTGAGGAGGGGGCCGTGACAGT |
| 155 | chr16: | TGTCCGACAGGCACACAGAGCGCCGCCAGGCACGGCCCTCATTCTTCACCCCGAGCTCCCGCAAGGTCGGCGAGGAGGCTGGAGCAGC |
| | 85469900- | GGGTAGGAAGCGGGCCGAGGCTCCCCCGACGCTGGGCCGCAACTGTCATCGCAGATCCCTGAAAAACGAGCTCTGTAATCGTTGCCGTC |
| | 85470200 | AGCGGGTGTACAATTGCAGCCTTATGTTTCCTGCCGCTGTTTACCTTCCTGAGCGGCGCCCAGAGATGCACACACGCTGCCCTGAAGCGG |
| | | GACGTGACCTCTGGGCACCTGTGAGGTCCTGGG |
| 156 | SLFN11 | GTCGGCTCCTGCGCTCCCAACGGGGTGGCCGTTTCCTTCCTCGCACCCTCTTCTCTCCCGGTGCCTGCGGTCCCACCTTCCAGATACCCC |
| | | TCGGAGAGTCCAGCTGAGCTCTCGCCAGAGCTTTCCCCTTCCAACCCGCTCGACTTGCCCAGATCCCAAGCTGGGCTTCTCTCTCCATCGC |
| | | CCCAGAAAGTGGGTCTTGGAGACCGAGGCAAGAATTTGGGCCTCCGCTTCTGTTCCAGACCCCGGACCCCTTGCCAAAATGCGGCAGATG |
| | | TGCAGATTGGGCCGCGCTTGGTTCCTGGCTGGGTTTATGGAGCCTGCGGCTGAGGCAGGCTCCGCAGACCCCGAGCCAGAGTGGGATTT |
| | | AACGGCGGCCGGTGCGCTGTGCTTGGTCAACCCCGGTAACCGTCACGCTGCTAGTGATATGAAAAAAACCTGCCAGCGTTCTGCTTTTCTG |
| | | CCCCGCTGCAGTCTTTAGCACCCGCCAGGATTCTGTCCGAGTGTTTGGA |
| 157 | DLX4 | TTTAGTGTGTGCATAAAACATCCCAGCTAATCTCAAATAGACTTTTCCTGAGCAGAGGCTGAAATTTGCAAGTAATGCAAAGAAGACTCC |
| | | GGGAGAGCGTCGCCGATGGTGGAGCGGGAGACGGGCGTGGGGAGCCCCACTGCAGTGCTGGGATCGAAGTGGTGCTGACCCCAAGACCTC |
| | | TCCCCTCCTCCTCCCCCGGGAGCTTCTCCAGGGTTATTTGGGAAATGAGGGGGAACTCCAATCCCTGAGAAAGCGCTCAGGGGCTTGCTG |
| | | AGGTGAGCGCAAATGGAAGCACAAGGCCGGGCTGGCCGTGGGCTCAGTAACCAGTCGGCTGCCCGGCTTGCGCCAGCACTAAATGCTCG |
| | | ATCAGAAAGAGAAAAAGAGGCGCAATAATTCCAAATTTCAGGAAAAGTCAAATCGGAGAGGGGGGACGCAGGTCTCTTCAGACTGCCCATT |
| | | CTCCGGGCCTCGCTGAATGCGGGGGCTCTATCCACAGCGCGCGGGGCCGAGCTCAGGCAGGCTGGGGCGAAGATCTGATTCTTTCCTTC |
| | | CCGCCGCCAAACCGAATTAATCAGTTTCTTCAACCTGAGTTACTAAGAAAGAAAGGTCCTTCCAAATAAAACTGAAAATCACTGCGAATGA |
| | | CAATACTATACTACAAGTTCGTTTTGGGGCCGGTGGGTGGGATGGAGGAGAAAGGGCACGGATAATCCCGGAGGGCCGCGGAGTGAGGAGG |
| | | ACTATGGTCGCGGTGGAATCTCTGTTCCGCTGGCACATCCGCGCAGGTGCGGCTCTGAGTGCTGGCTCGGGGTTACAGACCTCGGCATCC |
| | | GGCTGCAGGGGCAGACAGAGACCTCCTCTGCTAGGGCGTGCGGTAGGCATCGTATGGAGCCCAGAGACTGCCGAGAGCACTGCGCACTC |
| | | ACCAAGTGTTAGGGGTGCCCGTGATAGACCGCCAGGGAAGGGGCTGGTTCGGAGGGAATTCCCGCTACCGGGAAGGTCGGAACTCGGG |
| | | GTGATCAAACAA |
| 158 | SLC38A10 | CATGGTGCTTCAGGAAGGGAGGGGACGAGAGCCCTGGGCTTGTGGTGTCCACGTGGACAGCTAATGAGGAGCCTTGCCGATGAGGAGCA |
| | | TGCGTTCCCGACGGGGCGGCCGAATGCGGAAGGAGCCGCCATTCTCTCCGCCCTGACCGCGGGATTCTCTGCAGCAGATGAGAAACGGC |
| | | GCTGACTCAGCAGGGTCCCTCCCAGGCCCCGAGCGGTCATCTGGTGACCCCCGCGCTTCCCCCACGGCCCAGCCGGAGAAGGGCAAAG |
| | | GGAAGTCCCGGCTCCAAGGCGCACCCAGAGATGCGGTGCATGTGGCAGGATGGCCCAGCCCCGTCGGCAGCCCCAGCTTCCTGCCCCT |
| | | GGTTTCCTTCCTCCCACGGGCTACAGGCCTCTGATGAGCTTTGGAAAGCAGGAAACACACAGGCTAGTAACTATGAATGGGTCCAAAAAAC |
| | | ACTCCTTATTACTTTAAACTACTTAGGAAGAAGCACAGCGTTGCCAAACGCCAGA |
| 159 | S1PR4 | GCGCGGGGGGCCGGAGGATGGCGGCCTGGGGGCCCTGCGGGGGCTGTCGGTGGCCGCCAGCTGCCTGGTGGTGCTGGAGAACTTGCT |
| | | GGTGCTGGCGGCCATCACCAGCCACATGCGGTCGCGACGCTGGGTCTACTATTGCCTGGTGAACATCACGCTGAGTGACCTGCTCACGG |
| | | GCGCGGCCTACCTGGCCAACGTGCTGCTGTCGGGGGCCCGCACCTTCCGTCTGGCGCCCGCCCAGTGGTTCCTACGGGAGGGCCTGCT |
| | | CTTCACCGCCCTGGCCGCCTCCACCTTCAGCCTGCTCTTCACTGCAGGGGAGCGCTTTGCCACCATGGTGCGGCCGGTGGCCGAGAGCG |
| | | GGGCCACCAAGACCAGCCGCGTCTACGGCTTCATCGGCCTCTGCTGGCTGCTGGCCGCGCTGCTGGGGATGCTGCCTTTGCTGGGCTGG |
| | | AACTGCCTGTGCGCCTTTGACCGCTGCTCCAGCCTTCTGCCCCTCTACTCCAAGCGCTACATCCTCTTCTGCCTGGTGATCTTCGCCGGCG |
| | | TCCTGGCCACCATCATGGGCCTCTATGGGGCCATCTTCCGCCTGGTGCAGGCCAGCGGGCAGAAGGCCCCACGCCCAGCGGCCCGCCG |
| | | CAAGGCCCGCCGCCTGCTGAAGACGGTGCTGATGATCCTGCTGGCCTTCCTGGTGTGCTGGGGCCCACTCTTCGGGCTGCTGCTGGCCG |
| | | ACGTCTTTGGCTCCAACCTCTGGGCCCAGGAGTACCTGCGGGGCATGGACTGGATCCTGGCCCTGGCCGTCCTCAACTCGGCGGTCAAC |
| | | CCCATCATCTACTCCTTCCGCAGCAGGGAGGTGTGCAGAGCCGTGCTCAGCTTCCTCTGCTGCGGGTGTCTCCGGCTGGGCATGCGAGG |
| | | GCCCGGGGACTGCCTGGCCCGGGCCGTCGAGGCTCACTCCGGAGCTTCCACCACCGACAGCTCTCTGAGGCCAAGGGACAGCTTTCGCG |
| | | GCTCCCGCTCGCTCAGCTTTCGGATGCGGGAGCCCCTGTCCAGCATCTCCAGCGTGCGGAGCATCTGAAGTTGCAGTCTTGCGTGTGGAT |
| | | GGTGCAGCCACCGGGTGCGTGCCAGGCAGGCCCTCCTGGGGTACAGGAAGCTGTGTGCACGCAGCCTCGCCTGTATGGGGAGCAGGGA |
| | | ACGGGACAGGCCCCCATGGTCTTCCCGGTGGCCTCTCGGGGCTTC |
| 160 | MAP2K2 | GGGCGGGTTGCCACACTGTCCCCTTTCTGCATGGGAGGAAGGGGGCTCGAGAACTGAGTCAGCCACACAAAACGAGGATGGACAGAACT |
| | | CCTGAGTAGCGAGGGTGCCTGCCGGGCGCGAGGAGGAGGGGGAAGACGAGGAAGACGAGGAGGAGGAATAGGGAGCACCACATGACA |
| | | GAGGGGCTGCCTCAGACCACAAAGCGCTTCCTCATCCTTTCCTCGCCCTTTGATGCCGCCGGCAACGTGACTCTGCGAGCAGCGGGGCAG |
| | | ACGCCAGGTCTCCCTCGCAGGCGGGAAAGGGGCTCCAAGGCGGGTGCTGCCTTGCTCGGGTCACATGGCTACGTGGGGGCCTTGCTCAAA |
| | | TTCACTTCCTGCCTTCATTACAAAACTGTCAAAGGGGATCGCACGTTTGCAGGGTGTCACCCAAGCATTCTGGTTTTGCAAACGACGCTGT |
| | | GCGGCAGGCGGTCTGATACCTGATGAGCTCGGTGTGGCGGGGTCGGCAGCATTTCCTCCGGGGTTTTGAGCTCTGGCCACTTCTCCTTTT |
| | | GTTCCACCCAATCTCACCCACTTCTGGGCTTCGAGGCCAGAGTGTCTTAACAAGGGGGCACGT |
| 161 | UHRF1 | GAGCGAGACTTTGTCTCAAAAAAAAAAAAAACCAAATAAATTGAAAGCTGAGAAATTCAGAGCACAAGAAGACAAGCGCGCCCCCTCTTTT |
| | | AGCTGTCAACATGGCGGAGCCGTCCCTGGTGACGCAGCCTCCAAAGGCCTCCCTGTGCCCTCCTGAGACCGCAAGAGGGAAAGTGGCAGC |
| | | GACAGTGATCGTGGTGTCTTTGTGGCGGTTGTGTTGACCTCACTGACCCCCGAAGTGCCGCTCTAGGGTCTGTCCTCAGCGGTGACCCGG |
| | | CCGGGTCGAAGGGCAGAGTTCCGCTGTCACTAGCCCTCCACCCGTCCTGTGTGCTGGGATGCCCTCGCGGCGCCGTCCACGCCACCGCC |
| | | GCCCCCTCTTGTGGGTTCTGTCTCCTCCGTGTCTAGGATCCTCCTGCATCCGTTTTTCCTTCCTCCCTTCTCTCCCTCCGTCTGTCTTGCC |
| | | CGCACCTGAGGTTGTCGCAGAGGCGCTGAGACGGGCCAGCAGGAGCTGT |
| 162 | DEDD2 | TGCTGTCCCGGTCCTGTCGCAGTCCTCAAAGATGCTAGAGTGACAGTCCTCTAGGGGTAGAGATGGTCGTCCTCCCAGGAGAAGGTGGCC |
| | | CGGAGACTTGGAGGTGGGATCAATCCTGCCAGTCCTGGATCAGGAGGCCTCTGTCGGGCGCCGCCCCCCTTCCTCCTCCATCAGCAACAG |
| | | GCGGCGCCGGCCAGCCTCATAGTCAGCCTCATCCACACTGACCAGCAGGCGAACAGCCTCCCGGCCCACAGCCTCTCGCAGGGCCTCAG |
| | | TCAGGAACACGCCCCGCAGGGCCTGCAGCAGGGCGCCACTCAGGTAGTCGCCCCAGAAGGCGTCCAGATAGGAGAGCTCTGAGAACTTG |
| | | ATGTCACAAACCACAGAGCCCAGGTCCCTTGAGCGCAGCACTGCGGTGGCCTGCCCAAACACGTCCAGCTGCCGCGCCAGCGCCTGGGG |
| | | CCGCCGGGATGCCACGCCCTGCTCCAAGGCTGGCCCATGCTCGCAGTACTCTGCTCGAACCCGGAGCCGGATGTCTGCAGGGGAAGGAG |
| | | GGATTTGTCAGGGAGGGGGCCAACACTAGACACACTTATGGGGAACGCCACCCTTCCTCCCTCC |
| 163 | CDC42EP1 | TGATGCCCGGCCCCCAGGGGGGCAGAGGCGCCGCCACCATGAGCCTGGGCAAGCTCTCGCCTGTGGGCTGGGTGTCCAGTTCACAGGG |
| | | AAAGAGGCGGCTGACTGCAGACATGATCAGCCACCCACTCGGGGACTTCCGCCACACCATGCATGTGGGCCGTGGCGGGGATGTCTTCG |
| | | GGGACACGTCCTTCCTCAGCAACCACGGTGGCAGCTCCGGGAGCACCCATCGCTCACCCCGCAGCTTCCTGGCCAAGAAGCTGCAGCTG |
| | | GTGCGGAGGGTGGGGGCGCCCCCCCGGAGGATGGCATCTCCCCCTGCACCCTCCCCGGCTCCACCGGCCATCTCCCCCATCATCAAGAA |
| | | CGCCATCTCCCTGCCCCAGCTCAACCAGGCCGCCTACGACAGCCTCGTGGTTGGCAAGCTCAGCTTCGACAGCAGCCCCACCAGCTCCAC |
| | | GGACGGCCACTCCAGCTACGGTGAGGGCCTGGGCCATCTTGGCCCACTTTTCAGA |

TABLE 4C

| SEQ | | |
|---|---|---|
| ID | GENE | |
| NO | NAME | SEQUENCE |
| 164 | chr21: | GGCCGGGCAAAAAGCCGCCGCAACAAAAAGCTGCGCTGACGGGCGGAAAAAGCCGCGGCGGCGGAGCCAAAAAGCCGGGGCGGCAAAA |
| | 9906600- | AGCCACGGTGGCGGGCGCAAACAGCCGCAAAAAGCCGCGGTGGTGGGGGCAAAATCAGTGGGAGCAGGGGCAAAAAAACACAAAAAGC |
| | 9906800 | CGCGGCGGCGGGGGCAAAAAGCCA |
| 165 | chr21: | TGGCTTTGCTGGAGTGTGATGTGATAGGAAATGTGCAGCCAAAGACAAAAGAAGATGTAAGTAGGCTTGACTCATTGCAGCTAAGAACC |
| | 9907000- | CAGATGTTACCTTGAGGGTATTAACTAATAAGCAGTTTAAATCAGAATGGCACATTCTGATTTGTTTTTTGTATGTTCACATTTGGCAG |
| | 9907400 | GCATAGATACTGTTTGAAAAGAGAAAAGTCAGTACATAGAGGTAACAAGCTTAAATATGTGCCAAGTCTAGAAACAAGAGACTAGGGGG |
| | | ATAAGGACCTTTCGAAATTAAATGCAAGATTTGAAAACTGATTGGCTGGGGGATGAGGCAAAGGCAGGTCTTTAAGGTCAATCCCTGTT |
| | | TTGCTTTAAGTTGTTAGCGGGTGGTTTTATCATATATTGTAGAA |
| 166 | chr21: | TTCCTGGGAATGTCAGCTAACCTGAGCCTAGGGGCCTGAGCCCAAGGGCAGACTGAGGCTCCCCCAGCACAGGGAGGTGCTGCCTGTGA |
| | 9917800- | CAAGGGGTAGTGCTGGCACAGTGCAGGCTACTCCCTAGAAAGATCAGCTTGAATATGCAGGAAGAGCAGGACCCTCGGGCTGAGGCAGA |
| | 9918450 | GGTGGAATGGGAAGTGCATGGTGGTAATTTAGTTCTCCAGAGGCCAGAAGTAGGAGGAGCGGTTGGAATGCTGATGGCCCAAAGGGAAAC |
| | | CCTGGACTACCCTGGCCTCCCACAGGACTCTCATAGTAATTGCGGCTCCCTGCAGTGGTGAGGCCAGAAGGAGTGTTGCCCAATGCTGTC |
| | | ATCATCCAGTCCACCCCCCACCCACCATCAACAGATGAGTATGGTCATGAGTGTGGTCACCTCATCAGTCATTTGCTCAGTTGTGAAAAA |
| | | GAAATTGTTCAGAGAAGAGCAAAGTGTTTTTCCATGAGCCAAAGGTCAGCCAAGTTATGCTAATGAGGAGGACTGGAGACAGCGTGTCAC |
| | | AGACACCGAGAAGGAGCACTGGGCAAGGGCACTTCTCCCAGGGCAGAGCCCACAAGAAGCGTCCTGGCACCAGACACTCAGGGAACTGAA |
| | | GGCTGGCAGGGGCCCGCCCAGT |
| 167 | TPTE | TCCCCCCAGCTGGGTATAAGCAAACTTTCCTGTCTATGGGCCGCAGAGACCACCATCTAGTTCCCCCGCCAAAACTTTACATGATTTTAA |
| | | TTCTCCTGATGAAGATGAGAGGATAACAGCCAACAGAGAGGGCAGAGGATGGGATGGGACTCCCTTGCTCAGAGACCTCACCTCTAGGTC |
| | | TTTACCTCCTATTGAGAATAAGTCAGTTCTGTAGTAAGAACTCTGTGTCCACGGCAACCCCAAACAGAATCCTAGCGCTCTTGTGATTCT |
| | | TGTAGAATGGGGAATAGAACGAGCTTGGCCCAAGACTGCACAGACTTAAAAACATACTATTCTTTGAAAATGGCAATCATTAAAAAGTCA |
| | | GGAAACAACAGGTGCTGGAGAGGATGTGGAGAAATAGGAACACTTTTACACTGTTGGTGGGACTGTAAACTAGTTCAACCATGGTGGAAG |
| | | TCAGTGTGGCGATTCCTCAGGGATCTAGAACTAGAAATACCATTTGACCCAGCCATCCCATTACTGGGTATATACCCAAAGGACTATAAA |
| | | TCATGCTGCTATACAGACACATGCACACGTATGTTTACTGCAGCACTATTCACAATAGCAAAGACTTGGAACCAACCCAAATGTCCAACA |
| | | ATGATAGACTGGATTAAGAAAATGTGGCACATATACACCATGGAATACTATGCAGCCATAAAAAATGATGAGTTCATGTCCTTTGTAGGG |
| | | ACATGGATGAAATTGGAAATCATTCTCAGTAAACTATCGCAAGAACAAAAAACCAAACACTGCATATTCTCACTCATAGGTGGGAACTGA |
| | | ACAATGAGAACACGTGGACCCAGGAAGGGGAACATCACACTCTGGGGACTGTTGTGGGGTGGGGGGAGGGGGGAGGGATAGCATTGGGAG |
| | | ATATACCAAATGCTAGATGAGGAGTTTGTGGGTGCAGCGCACCAGCATGTCACACGTTTACATATGTAACTAACCTGCACATTGTGCACA |
| | | TGTACCCTAAAACTTAAAGTATAATAAAAAAAATACTGTTCTGCCATACATACAGATACTCATTAAAGATGAGGGAGAAGGGCATGGGGT |
| | | GGGGGAGAATGTACCAAAACCAAAGACCACAGGATAATAACCTCAGAGCAGAGACTATCTCTCTAGTTATTTTTTCTTTTGTATGTAATG |
| | | GAGAGGATTATTATTTACTCTGATGAAGAAGTTTACATCAAGTGTTCAGCTTCCTTTGTGGGTTACAGAGAATAACCAGAGGGCTCAGTT |
| | | ATGCTCTCTGAATAACTATGTTTGCTTAGTGTTTTCTAAACAATATTAAATTTCACTAAAATAGACAAGGTTGATAGGACTTGGGGGCAT |
| | | AACTCATTGACTCAAGCTATCATTTTATAGGATTGTGAGAAAACAAATAGATGAACATTTAAAATACACTCATATTCTCGCTAGAAAAGA |
| | | GGATTTTGAATATTCTTACATCAAAGACATGGTAAATGTTTAAGGCAATGAATATGCTAATTACCATGATTTGATCATTATGCAATGTAA |
| | | AATGTACTGAAACATCACATTGTACCTCATAAATATGTACAATTTATTATGTGCGAATTAAAATTTTGAGTATAAGAAAAAATAAACTTC |
| | | AATTGTAAGAAAACAACCCAACTTTTAAAAAACGGGCAAAATACGTGAACAGATACTTCACTAATAGAGATTTGCAACTGGCAAATAAGC |
| | | AAATGAAAAACTGGTCATCATCACTATCTATTAGAGAAATGCAGATTAAAACTACAATAAGAAACAATGCTGCCCGTCCAGACGCATTGT |
| | | TTTGACCGTTTCCAACTTGTCCCAGCCCTTCCCGGGGCATCGCTGGGGACCCTACGCCGACGTCCCCCCTCCGCCCGCGCCCCAAGGGCC |
| | | GACTGGGCAAATTGGGAGACCCGCCCCGCGGGGCGACCCAACTTTTCGGAACAGCACCCCACCGCCCACCCCCGCAGACCCCCGGACCCC |
| | | CGCTCCCGGCGGAGACTCAGGGAACCCCGCACCCCAAGCCCTTCTAAATCGTGCAGCGTGAGTGTGACGGCCAAGAGCGGATGCAGCCCG |
| | | GGATCGCCCGCACCTTCCCGTGGGCGGAAGCGCAGGAGCCAGCTGGGGAGGGGGCGCCCTAGAGGAGCGGCTAGAAAGCAGACACGGGGA |
| | | ACTCAGGTCATCCTGGGGGGGGACAAGACAACGAGAGCCGGGCGCCTCGGGGGCGGCGCGGGAGCCTCCGCAGGACCGGGCGGGCGCCCC |
| | | GGCTGGCGCGGGCGGGGGGCGCGCCCCCTTTACCTGCGGCTCCGGCTCCTAGGCCATTTCCTCACGCGGCGGCGGCCGGGACTGAGCTAA |
| | | CACCACTCAGGCCGGCCGGGTTTGAATGAGGAGGAGCGGGCGCGGAGAGGAGGGGACGGGGAGGGCGGAGGGAGGGAGGGAGGCGTCGCG |
| | | GAGTTTTTCTCGGCCTTTTGTGCGGACACCTCCCGGATTCCGCGCCCGCACCCGGCCCCCCAAAAGACACGGGGAGCCGCGGGCGAGGGG |
| | | TTCAGCCATCCGCCGAGGCGCCTAGTGCCTTCGCGCCTCCAAGACCCCCCCCCAACAAAAAGGAGCGTCCCCCACCCCTACCCCCGCCCG |
| | | GAGGACTTAGGGCCTGGGCTCACCTCGGGCGCGGAGCTAAGTGTAGGCGCCGGGGGTCCCTAGAGCCGCCGGGGCGCAGCGAGTCCGGCG |
| | | CTGGGTAACTGTTGGGTCAGAAACTGTTCAGGTAGCAGCTGTTGTGCCCTCCCTTGGCCCCGCCGCTCGGAGACGCCCCGCCCCCTGCCT |
| | | TGAACGGCCGCCCGGCCCCGCCCCAGCGCCCACGTGACTAGCATAGGCGCGCCCCCGTTCCGCCCGCCGCCGCAGACTCCGCCTCCGGGA |
| | | CGCGAGCGAGCGGCGAGCGCGCGCACTACCAGTTCTTGCTCGGCGACTCCCGCGCACGCGCGCGCCGTGCCACCCTCCCCGCACCCCTCC |
| | | TCCCGCCATCCGGCTTAACGTGGCGGGCGCGCGCCGCGGCAGTAGCCGTGACAGGTACCCGGCGGGGCGGGGGGGGAGGGGGTTGGCCCG |
| | | CGAGGGTGTGCGCAGGCACAGACCCGGGTCCTGTCCCCGCCGCCCCCTCCTCTGCAAGGTGTGCCTGGGCGAGGGGAGGGGCCCGCGGCC |
| | | CGAACCCCTGGGTCACCCCCGAATTACAAACAAAAACCTTAACGCCATTGCTCGCGGGTTAGAAGGCAGCTGTGCGTGCTCAGGAAAAGA |
| | | AGCCACGCACAAGAGACCGCACGCGGCGTGGATACAGTGACACGAAACACCCAAAATCTCTTTTGAAAGGGAAACCAGGCACAGTGGCTC |
| | | ATGCCTATAATCCCAGCACTTTCGGGGGCCAAGGCGCTCACCTAAACCCGAGAGTTCAAGACCAGCCTGGGCAATACAGCGAAACCCTGT |
| | | CTCTACGAAAAATATAAAAATTAGCTGGGCATAGGGCTGGGCACGGTGGCTCACGCCTGTAATCCCAGCATTTTGGAGGCCGAGGCGGGC |
| | | GGATCACGAGGTCAGGAGTTCCAGACCATCCTGGCTAACACAGTGAAACCTTCTCTCTACTAAAAATACAAAAAAAATTAGCCGGGCGTG |
| | | GTGGCAGGTGCCTGTAGTCCTAGCTACTTGGGAGGTTGAGGCAGGAGAATGGCATGAATCAGGGAGCGGAGGCTGCAGTGAGCTGAGATT |
| | | GCGCCACTGCACTCCAGCCTGGGGGACAGAGTGAGACTCCGTCTCAAAAAAAAAAATAATAATTAGCTGGGCATGGTGGCTGGCACACAT |
| | | GGTCCCAGCTACTCAGGAGGCTGAGGTGGAAGGATCTCTTGATCCCGGGGAGGTCAAGGCTGCAGTGAGCCAAGATGGCATCACCGCACT |
| | | CCAGCCTGGGCCACAGACCCTGTCTCAAAAAAAAAAGAGAAAGTGGGGAAGAAAATGTAATACAAATTAATATACCAACAGCAATTAGTG |
| | | AGTACTTTTTCCATGGAGCTGGGAGAGGGAATAAATGTTTGTAAAATTAAAATGTTCTACGCTAGAAATCAACTTTCCTTCTATGCTTTC |
| | | TTTACTTCACCCCTTATAGCTACTTAGTAAATCTCACAAATCCTATCCTTCTGATCTCTCTGAAATGTATGTACCCTTTCCCTTCTATTC |
| | | TCACCACCCATGTTTCTTTGTTTCCTTCTAGCCTGTGTAATAATCTCATAATCGCACCTCCTGTACCTGCCTTCTTTCTAGTCCAGAATA |
| | | CGTTTTCCTAAATTCCACCAATAACCATCCTGCTACTGCTTTGTGTGAAATTCTCCAAAAAAAATTTTACTTTTCCAAAATAAGTCAGGC |
| | | TCCCTCTCTTAGGATACAAAACCACACCATGGTCCCAGCCAATCTTTCAGCCTGATTCACTCAGTATATATTTATTGACCTCTCCTTTCT |
| | | CCCAAGCACTTGGCTAGATAATAATTAAAGAGTGCGGCACAAAACAAATTGGATTCCTCCCCTCATGGAGCTTGTATTTTCACAGGAAGC |
| | | ACAGACATTAAATAAATTAAAACACAAAAAAATAGACAAGCATATAATTACAGTATGTATCCTAGAGAAATATCACTCATGCAGAAAGCA |
| | | TACACAAGGATGCAGCACTGTTTCCAATAGCGAAAAGCTAGAAACAACCTACATGTTCACCAAAAGAAAATGGCCACATAAACTATACCA |
| | | TATCCAAATTATCCAAATTTTAGAATATAGACAACAGGTTGGGCGCGGTGGCTCACACCTGTAATCCCAGCACTTTGGGAAGCCGAGGCG |
| | | GGTGGATCACAAGGTCAGGAGTTCAAGACCAGCCTGGCCAACATGGTGAAACCCCGTCTCCTCTAAAAAAACAAAAAAATCAGCTGGGCA |
| | | CTGTGGCAGGAGCCTGTAATCCCAGCTACTGAGGAGACTGAGGCAGGAGAATCGCTTGAACCCTGGAGGCAGAGGTTGCAGTGAGCCAAG |
| | | ATCGCGCCACTGCACTCTAGCCTGGGTGACAGAGCAAGACTCCATCTCAG |
| 168 | chr21: | TGTAGGAGTCCTCCGGTGCTGGAGTCCAGAGCACAGTGAGGCTGGGTCCTCCCGTGCCATAGTGTAGGGCATGGCGGGACAGGGATCCT |
| | 13974500- | GCCCTGCGATAGTCCAGTGCTTGAGTCCGCAGTAAGGCAATGGTCCTCCAATGCTGGAGTTCACGGCGTTGTGGGGTCGGGGTCCTTTGG |
| | 13976000 | TGACTTAGTCCAGGGCGTACCAGGGCGGGGGTCCACAGTTGCCATAGTGAGGATCTTGGAGGAAGGTGGTTCCTGCCTTGCTGTAGTCCG |
| | | GGGAGCAGGGGGCAGGGGTCCTCTCTTGTCAGAGTCTCTGGCGCGGGGTGGGGGTGGAGGTGGGGGTTTTCCTATGCGATAGCCCACG |
| | | GGTCGGTGAAGCCGGGTCCTCCCGTGCCTTTGTCCAGGGCGCAGGGGGGCGAGGGTCTTCGGTGGTGGAGTCCGCGGAGCGGCAGGAC |
| | | GGGGGTCCTCCAGTGCCATATTCCAGGGCGCGGCGGAGTGGGGGACCTGTCCTGCAGTGGTCCAGGGCATGTGGGAGTGGTGGTCCTG |
| | | CTGTGCCTCAGTCCAGTGCGCGGTGGGACGGCGGTCCTGCTGTGCTGTAGTGCAGGACGCGGTGGCGCAGGGGTAGTCCAGAGAGCGC |
| | | CGTGGCAGGGGGTCCTCCAGTGCTGGAATCCAGTGCAAGGCGGGTCAGGGGTCTTACCGTGCCGAAGTCGGTGGCAAGGGTCCTCCCGT |
| | | GCCATAGTCTAGGGGGCGACGGGGCAGGGTTCTCTAGTGCAGGTGTCCAGGGTGTGGCAGGGCAGGAGTCCTCTTGTGCAGGAGTCCAG |
| | | GACGTAGCCGAGGAGTCCTCCAATGTCAGAGTCCAGGGCTCTGCGGGGCCGGGTTCCCCCATGCCAGAGTGTAGGGCGCGTTCAGGTGA |
| | | GGGTCTTGGCGTGCAGTAATCCAGGGTGCGGTGGGGCAGGGGTAGTCCAGACCTCCATGGCGGGCGTCCCTCTGTGCAGGAGCCCAGT |
| | | GCCTGGCGGATCGGGGGTCCTTCTGTGCTGTAGTCCAGGGCACCGCAAGGTGTGGGTCCTCTGGTGCCCTAGTCCAGGGGGCGGCGAGT |
| | | CAGAGGTTCTCCCGTGTCTCAGTCTAGGGCCTGGTAGGACTGGGGTCCTGGAGTCCACGTGGTAGCCCAAGTTGCCGCAGGACCAGGTA |
| | | CTCTGGAACCACAGTCCAGGGCGCTGAGGGGCAGGAGTAGTTCAGGGCGAGCCGGGGCCCAGGTCCTCGGGAGCCAGAGTCCAGGGTG |
| | | TGGAGGGGTGGGGGTTCTGCAGTGGCACAGTCCAGGACACCGCGGGGCGGGACAGGGCGGGGATCCTCCCGTGCCTTAGTCCAGGGCT |
| | | GAGCCGCGGGAGAGGTCCTTCAGTAGCACAGTCTAGCGCACGGCGTTGCAGGTGTCCTCCAGTGCCTGAGGCCACGGCAGGTCGCGGGT |
| | | CCCACTGTGCTCTAGTTCAGGGCGGAGTGGGTCTGAGGTCTTCTCCTGCCTCAGTCTAGGGCGCTGGAGAGCGGGGATCCT |
| 169 | chr21: | GGGTTGGTCCTAGAAAGCGTGAGGATCGCCGAGTGCACTGCCCTCCCAGCCTAGGGTCCACTCTTCCTTGGCCCGAGCCCAGAGCTCGG |
| | 13989500- | GGTTTCAGGCGCTGGGCCCTGTGCAGCTGCCCAGAATAGGCTGAGCGGCAGGTTCCCGCCCTGGCAAGGGATCCAGCAGTGGAATCCTC |
| | 13992000 | ACTGCTGTTGGCTGCGGGCAAGGTCAGCGGGGTTTCCATCGCTGCTGGTGGGAGCCACCTGGCGGTGGTAGCTGCAAGTGAGCGCGTGG |
| | | CAGAGACTGGCAGGGCTGGTCCCAGACACCCTGAGGGTCTCTGGGTGCATCGCCCTACCACCCTAGGGTCTGCTCTTCCTTAGCCTGCTC |
| | | CCAGGACGCGGTGTACGAGGGCTAGACTCTGAGCAGCCTCCAGGATGGGGCTGAGCAGCGGATTCCTGCCCTGCTGCAGCTACAGTCTG |
| | | AATTAGGCGCCACCGCAGTATCTGGCCCTGGGGTACGTGCTACTGGGTGGCATGGACAGAGATGGGGGCTGCCACAGCTGCTATGGGGC |
| | | TGAGCAGCCGATTCTCGCCCTGCTGCAGCGGGCGACCGCTGCAATCCCCAGCGCTATGGGACCGACCACCTGACTTAGATGCCTTGGAG |
| | | GCATCCGGTCCTGGGGTCTTGCTGCTGGTGTCTGCGGGCAGGGTCACGGCTGCCACTACTACTGCTGTGCGCCATGGGCAGGTGCCAGC |
| | | TGCAGCTGAGTCCGAGGCAGATGCTGTCAGGGCTGGTCTGAGGTTGCCTAAGGGTGGCTGAGTGCACCACGCTTCCACCCCAGGGTCCG |
| | | TTATTCCTAGGCCGGCTCCCAGATTGCAGGGTTGTGGGCGTTGGACACTGTGCAGCCATGAGGATCTGGTTGGGTGCAGATTCCCGCCCT |
| | | CCTGCAGCTGAGAAGCCAATCTCATAACAGGCGCTGCAGTGACCTCTGGCTCTGCGGTCCGCGCTGCTGCTGGAGCTGGCAGAGAACAGA |
| | | GCTGCCACCGCTGCTGCTTCCAGGAGTGTGCAGCTGGCAGCTGCAGCTGAGCCCGTGGCGGAGGCTGGAAGGCCTTATTCCAGAAGCCT |
| | | TGAGGGTCCCCGAATGCACCGCCCTCCCACCCTAAGGTCCAGTCTTCCTTGCCCGCGCCCAGAGAGTTGGATTGCAGGCGCTGAGCACAG |
| | | TGCAGGTGCTGGGATGGGGCTAAGCTGAAAGTTTCCGCCCTCTGGCTGCTGCGGGGCCGACAGCCTGAGTTATGCGCCGCGGCGGCTTT |
| | | TGGTCATGGGATCCGCACTGCCGGTGGCTTGCACAGGGTCGGGGGCTGCCACAGCTGCTATAGTTCACCGTGTGCACGTGGCAGCCGCC |
| | | CCTGAGCCCACCGCTGAGGCTGCAGGGCTGGTCCGGTCCCAGACGGCCTGAGGGCCATTTGCCCGCGCCCAGATCCGGGTGGCTGCGC |
| | | TGGGCACTGTGCAGCCTCCCGGAATCCGCTGAAGGGCACGTTCCCGCTCTCCTACAGCTGTGGGCCGACTGCCTGATTTTGGCCACTAGG |
| | | TGGAGTCTGGCTCTAGGGTTTCGAGGCCGCTGGTGTTGGTGGGCGGAGTCCGGGTTTGCCACCGCTGCGCTCCATGAGCAGGTAGCAGC |
| | | TGCAGCGGAGCTTTAGACCGAGGCTGGCAGGGCTGGCCCCAGACGGCCTGAGGGTCAGGGAGTGCAGGGTCCTCCCACCCTAGGTCCG |
| | | CTCTTCCTTTCCCCTTACCCAGAGCGGGTTGTGCGGGCTCTGGGCTCTGTGCCGGCGCTGGGCTCTGTGCAGCCGCCGAGATGGGGCTG |
| | | AGCAGCGGATTTCCTCCCTGCTGCAGCTGGAGGACGATTACCTGCACTAGCCGCTGAGGCGGCATCTGGCCCTGGGTTACTGCAGCTGGT |
| | | GACGCGGGCAGGGTCAGGGTTGGTTGCAGGTGGCAGCTGCTGCTAAACCCATTGCGAGCCTCAGGGTCACCAAGTTCACCGTCCTTTCAT |
| | | CATAGTATCTGATCTTTGGCCCGCGCCCAGAGTGCGGACTGGCCTGCGCTGGGGACTGCATAGCTTCTGGGGGCCGGTCAGCGCCAGTTT |
| | | CACGTCCTCCTGCAGCTGCGTGGCCTAAGGTCTTAGGCGCCGCGGCGCTATCTGGCCCTGCTGTCGACGCTGCTGGTGGTGGGGACAGG |
| | | GTCAAGGGTTGCCACTGCTGCTCCCGTGCGCCATCGGCAGGTGGCAGTTGCAGATGAGCCCACAATTGAGGCTGTTGGGGCTGCTCCCA |
| | | GGTTGTTAGAGGGTCGCCGAGTTCACCGACATGCCACCCTAGGTTACGCTCTTGGCCCGCACCCAGAGCGCCGGGTTACGGGTCCTGGG |
| | | CCCTGTGCAGCCACGGGGATGGTGCTGAGTGCAGGTTCCCGTCTTCCTGAGATGCGGGGCGACCACTGGAATTAGCCTCTGTGGTGGTAT |
| | | CTGACCCTAGGGTCCGAGCTGCTGGTGGCGTGGGCGGGGTCGAAGTCGCCTCTGTTGCTGCGGCGTGCCATTTGCACCGTCCTCTGGTA |
| | | C |
| 170 | chr21: | AAATACTCTACTGAAAAAACAGAAATAGTAAATGAATACAGTAAAGTTTTAGAATACAAAATCAGCATAGAAAAATCAGTCGCATTTCTA |
| | 13998500- | TACCCAACAGCATACCATCTGAAAAAGGAATCAAGAAACCAATCCCATTTAAAATAGCTATAAAAAAATGCCTGGGAATAAACTAAGCCA |
| | 14000100 | AATAAATATGTCTAAAATGAAAACTATAAAACATTGATAAAAATCAATTGAAAAAGATACAAATAAAGGGAAAGTTATCCCATTTTTATG |
| | | AATTAGAAGTATTAATACTGTTAAAATGACCATCATACTCAAATCAGTCTATAGGTCCAATACAATCTCTAACAAATTTCCAATGTAATT |
| | | CTTCAGAGATGTTAAAAAAGGTTTTAAAAATCGTTCTGCGGATGTTAAAAGGATTTTTAAAACGCTTTTTTCGTTCTGCAGGCGAAGGCT |
| | | GTGGCCGTGCTCCCGCCGGCCAGTTCCCAGCAGCAGCGCATTGCCCCTGCTCCACGCCTTCGCTCCAGGCCCGCAGGGGCGCAGCCCCGC |
| | | GGGAATCAGCACTGAGCCGGTCCCGCCGCCGCCCCAGTGTCCGGGCTGCGACTGCGGGGAGCCGATCGCCCAGCGATTGGAGGAGGGCGA |
| | | CGAGGCCTTCCGCCAGAGCGAGTACCAGAAAGCAGCCGGGCTCTTCCGCTCCACGCTGGCCCGGCTGGCGCAGCCCGACCGCGGTCAGTG |
| | | CCTGAGGCTGGGGAACGCGCTGGCCCGCGCCGACCGCCTCCCGGTGGCCCTGGGCGCGTTCTGTGTCGCCCTGCGGCTCGAGGCGCTGCG |
| | | GCCGGAGGAGCTGGGAGAGCTGGCAGAGCTGGCGGGCGGCCTGGTGTGCCCCGGCCTGCGCGAACGGCCACTGTTCACGGGGAAGCCGGG |
| | | CGGCGAGCTTGAGGCGCCAGGCTAGGGAGGGCCGGCCCTGGAGCCCGGCGCGCCCCGCGACCTGCTCGGCTGCCCGCGGCTGCTGCACAA |
| | | GCCGGTGACACTGCCCTGCGGGCTCACGGTCTGCAAGCGCTGCGTGGAGCCGGGGCCGAGCGGCCACAGGCGCTGCGCGTGAACGTGGTG |
| | | CTGAGCCGCAAGCTGGAGAGGTGCTTCCCGGCCAAGTGCCCGCTGCTCAGGCTGGAGGGTCAGGCGCGGAGCCTGCAGCGCCAGCAGCAG |
| | | CCCGAGGCCGCGCTGCTCAGGTGCGACCAGGCCCTGTAGCTGTGACTTGGCTGTGGGGCTGGCCCGCCTCCCTGACCCCTGTCAGGCG |
| | | GAGCAGCTGGAGCTGACCCACGGGCCTGGGCTTTCGAGCGCTTTGTCCAGGCGCTAATGATGGGAAGGTGAAAGGTGGGGGTGGCCACA |
| | | CCCTGCAGTCAGGGTGGCAGGTGTCAGAGGCCACATGCAACCCACTGGTTTTGTCTTTTCCAGGATGCTGATAAGTTTCCCGCGGCCCCC |
| | | GGAGCAGCTCTGTAAGGCCCTGTAATTGCCTTTCGTTCCCTTCTGCTCTATTGAGGAGTGGGAAGATGACAAAGTGTTTTTGCTCAACCC |
| | | GAAGGAAAATGCACATGGGAGGACACACCGGGTTACTATTTGAGTAGCCCAGACAGGAGAGCAGCGGTCTGCT |
| 171 | chr21: | TGGGTGGATTGCTTGAGCCCAGGAGTTCGAGACCAGCCTGGACAAAATGGCAGAAACTCCATGTCTACAAAAAATACAAAAATTAGCCGG |
| | 14017000- | GCATGATGTTCTGCGCCTGTAGTCCCAGCTACTCAGGAGGCTGAGGTGGGAGGATCGCTTGAGCCCAGGAGGCGGAGTTTGCAGTGAGCT |
| | 14018500 | GAGATGTCACTGCATTCCAGCCTGGGAGACAGAGCCAGACTCTGTCTCAAAAGAAAAAAAGAAAAAAAAAAAAGAAAAGAAAAAACGAAA |
| | | TTGTATTCTGAATACATCTTCTAAAACACTACATTTACTTGCACTATATTAAACTGGTTTTATCCTGACCACAATTGCAGGTGAAAGATA |
| | | CCACTGTTGTTCTATTTTTCTGGTAAGTAGAGTGAGCCATGTCTTCCCCAGGGAAAGACGCCTCCTAAAAATTTGTAGGACCACCTTTGG |
| | | TTTTCTTCCAGATATTTTTTTTGTCATCGCTTTTCCTGCGCCCAATTCCCATCTGTCTAGCCCTTCTGCCTCCGCTGGTCTTTTTCGCGA |
| | | GCCTCTCCCCAGCCGCAGGTATTCGTCTGGGCTGCAGCCCCTCCCATCTCCTGGGGCGTGACCACCTGTCCAGGCCCCGCCCCCGTCCAA |
| | | CCCGCGGAGACCCGCCCCCTTCCCCGGACACCGGGTTCAGCGCCCGAGCGTGCGAGCGCGTCCCCGCTCGTCGCCCGGCTCGGCGTCGGG |
| | | AGCGCGCTCTGTGTGGTCGCTGCTGCAGTGTTGTTGTGGCTGTGAGAAGGCGGCGGCGGCGGCGGAGCAGCAGCCGGACCAGACTCCCTA |
| | | GTAGCTCAGGCGCTGCCCTGCGCCGGCCCTGGCAGGGAGCCTGGTGAGATGGTGGAGGAGGAGGCTGTGCCGTGGCTGGCCTTGCTGTGT |
| | | CCTGCTGCCTGGTTAGAACCCCATCCCCGTCCCCCGTCTCCTCCGGGGGGTGAGGAGGAGCTGGAAGAGGGGCCGGCCTCTGTCCGGCCC |
| | | GGCCAGGCGGCAGTCACCCTCTGAGGAGGCAGCGCCCGGGGAGGGGCCTCCCAGGCGGCCGCCGCCGCCAGGGGGAGGCGCTGGGAGTGG |
| | | GAGTGGGAGCGGGACCTCAGCTGCCAAGCTCGGCCCGGACCCTAGGTGCGGGGGAGGCGGGGTCCCGGGCTCGGGCTGCCTGCCCGG |
| | | ACCTGGCGGGGATGGGCCCGTGCGGCTCCGGGTGTGGGACGTACCCTCAGAGCGCCCGGGGTTATTCCCACTGACTCCAGGGAGGTGA |
| | | GTGTGCGCCCTTCGCTCCCTGCCGTGTCTGTGAGGGTCCATCGTTGCCGGAGACTGGAGGTCGGGGGCCATGGGAGCCCCGGGGCGAA |
| | | CGGTGCGGACATGGGCCTTGTGGAAAGGAGGAGTGACCGCCTGAGCGTGCAGCAGGACATCTTCCTGACCTGGTAATAATTAGGTGAGAA |
| | | GGATGGTTGGGGGCGGTCGGCGTAACTCAGGGAACACTGGTCAGGCTGCTCCCCAAACGATTACGGT |
| 172 | chr21: | GTCTCTAGGACACCCTAAGATGGCGGCGAGGGAGACGGTGAAGGTTGGCTCCCGCCTGTCTGGGCTCTGATCCTCTGTCTCCCCCTCCCC |
| | 14056400- | CTGCGGCCGGCTCATGGCCTGGCGGAGGCCCGAACCAAAGACCTCCGCACCGCCGTGTACAACGCCGCCCGTGACGGCAAGGGGGCAG |
| | 14058100 | CTGCTCCAGAAGCTGCTCAGCAGCCGGAGCCGGGAGGAACTGGACGAGCTGACTGGCTAGGTGGCCGGCGGGGGGACGCCGCTGCTCA |
| | | TCGCCGCCTGCTACGGCCACCTGGACGTGGTGGAGTACCTGGTGGACCCGTGCGGCGCGAGCGTGGAGGCCGGTGGCTCGGTGCACTT |
| | | CGATGGCGAGACCATGGAGGGTGCGCCGCCGCTGTGGGCGCGGACCACCTGGACGTGGTGCGGAGCCTGCTGCGCCGCGGGGCCTCG |
| | | GTGAACTGCACCACGCGCACCAACTCCACGCCCCTCCGCGCCGCCTGCTTCGAGGGCCTCCTGGAGGTGGTGCGCTACCTGGTCGGCGA |
| | | GCACCAGGCCAACCTGGAGGTGGCCAACCGGCACGGCCACATGTGCCTCATGATCTCGTGCTACAAGGGCCACCGTGAGATCGCCCGCT |
| | | ACCTGCTGGAGCAGGGCGCCCAGGTGAACTGGCGCAGCGCCAAGGGCAACACGGCCCTGCACAACTGTGCCGAGACCAGCAGCCTGGA |
| | | GATCCTGCAGCTGCTGCTGGGGTGCAAGGCCAGCATGGAACGTGATAGCTACGGCATGACCCCGTTGCTCCCGGCCAGCGTGACGGGCC |
| | | ACACCAACATCGTGGAGTACCTCATCCAGGAGCAGCCCGGCCAGGAGCAGCTCATAGGGGTAGAGGCTCAGCTTAGGCTGCCCCAAGAA |
| | | GGCTCCTCCACCAGCCAGGGGTGTGCGCAGCCTCAGGGGGCTCCGTGCTGCATCTTCTCCCCTGAGGTACTGAACGGGGAATCTTACCAA |
| | | AGCTGCTGTCCCACCAGCCGGGAAGCTGCCATGGAAGCCTTGGAATTGCTGGGATCTACCTATGTGGATAAGAAACGAGATCTGCTTGGG |
| | | GCCCTTAAACACTGGAGGCGGGCCATGGAGCTGCGTCACCAGGGGGGTGAGTACCTGCCCAAACTGGAGCCCCCACAGCTGGTCCTGGC |
| | | CTATGACTATTCCAGGGAGGTCAACACCACCGAGGAGCTGGAGGCGCTGATCACCGACGCCGATGAGATGCGTATGCAGGCCTTGTTGAT |
| | | CCGGGAGCGCATCCTCAGTCCCTCGCACCCCGACACTTCCTATTGTATCCGTTACAGGGGCGCAGTGTACGCCGACTCGGGGAATATCGA |
| | | GTGCTACATCCGCTTGTGGAAGTACGCCCTGGACATGCAACAGAGCAACCTGGAGCCTCTGAGCCCCATGAGCGCCAGCAGCTTCCTCTC |
| | | CTTCGCCGAACTCTTCTCCTACGTGCTGCAGGACCCGGCTGCCAAAGGCAGCCTGGGCACCCAGATCGGCTTTGCAGACCTCATGGGGGT |
| | | CCTCACCAAAGGGGTCCGGGAAGTGGAATGGGCCCTGCAGCTGCTCAGGGAGCCTAGAGACTCGGCCCAGTTCAACAAGGCGCTGGCCA |
| | | TCATCCTCCACCTGCTCTACCTGCTGGAGAAAGTGGAGTGCACCCCCAGCCAGGAGCACCTGAAGCACCAGACCATCTATCGCCTGCTCA |
| | | AGTGCGC |
| 173 | chr21: | TAAAAATAAATTGTAATAAATATGCCGGCGGATGGTAGAGATGCCGACCCTACCGAGGAGCAGATGGCAGAAACAGAGAGAAACGACGAG |
| | 14070250- | GAGCAGTTCGAATGCCAGGAACGGCTCAAGTGCCAGGTGCAGGTGGGGGCCCCCGAGGAGGAGGAGGAGGACGCGGGCCTGGTGGCC |
| | 14070550 | AAGGCCGAGGCCGTGGCTGCAGGCTGGATGCTCGATTTCCTCCGCTTCTCTCTTTGCCGAGCTTTCCGCGACGGCCGCTCGGAGGACTTC |
| | | TGCAGGATCCGCAACAGGGCAGAGGCTATTATT |
| 174 | chr21: | CGCCACCACGTGCGGGTAGCGCCGCATCGCCCCAGCCGTGTTCCTTGGTCTCCGTCTCCGCCGCGCCCGCCTGGTGAACTGGAGCACAGG |
| | 14119800- | GACCATAGTTCTGGAAATTTATCCTTTTTCTCTCCATGGATTCAGCAGCAGTGTCTAAAAGAAAAAAATTCATCAATCATTTATGTATAT |
| | 14120400 | TTTAATATAAAGGTAAAACACTGCGAACCAGTGGAACCGGATAGAAAGTAATTCAGTTTTACAGAACACAACTGTTTTTCAGGCTCTTTT |
| | | ATTAAATATAAAAGAGCCATATATATTTCTGTGGAATTCCCCTTTTACTTAAGAATTCATTATCAGCGAATTAGTTTAAGGAGGCTGTTT |
| | | TGTTAGAGGCTGTGGTTGCATTCAAAAATTGGAATAGGAACAATGACTTGTAAAAATTCAACATTTTATTTTATTTTTGAGATGGAGTCT |
| | | CGCTCTGTCGCCCAGGCTGTAGTGCAGTGGCGCGATCTCGGCTCACTGCAACCTCAGCCTCCCGGGTTTAAGGAATTCTCTGCTTCAGCC |
| | | TCCTGAATAGCTGGGATTACAGGCGCATGCCACCAAGCCCAGCTAATTTTTTTTGTATTT |
| 175 | chr21: | CCCTGAACAGTCAGAGTTTACTGCCCACTTTTGCTGGAGGAGAAGCTCCTGAACAACTAGAGAGACTGTGGTTCCCAAAGAGCAGCCTGT |
| | 14304800- | AGGCCTGAGGACTGCTCTATGACCGGCGTCAGTCCCTGCCTCCCTCCCTCCGTCCCTCCTTCCCTCCTTCCTTCCCAGGCCTTCTCTGAC |
| | 14306100 | TACCAGATCCAGCAGATGACGGCCAACTTTGTGGATCAGTTTGGCTTCAATGATGAGGAGTTTGCAGACCATGACAACAACATCAAGTGA |
| | | GTCCACTTGGATGCCCCCTGCACGAGGCACGACTCCCCCTCCTCGCTGCTGAAGTCCCATGGGGGCAGCTCCCTTAGTCCTTGCCGGGAG |
| | | ATAACAGGTGTTTCCAGTTGCATGAGGGTGCTGAGGCCCCCAGTGAGAACCAGGGGAGGAGCACTGAGGCCTCAGATGAGCACCGGGGGA |
| | | GGAGCCCTGAGGCCCCAGATGAGCACCAGGGGAGGAGCACTGAGGCCCCAGATGAGCACCGGGGGAGGAGCGTTGAAGCCCCAGATGAGC |
| | | ACCAGAGGAGGAGAGCTGAGGCCCCAGATGAGCCCCGGGGGAGGAGCTCTGAGGCCCCAGACGAGCACCGGGGGAGGAGCGCCGAGG |
| | | CCCCAGATGAGCACCGGGGGAGGAGCGCCGAGGCCCCAGATGAGCAGTGGGGGAGGAGCCCCGAGGCCCCCAGATGAGCAGTGGGCG |
| | | GGGCAGGGAGCGCCGAGGCCATCCCCCTTGCTCTTGCAGCGCCCCATTTGACAGGATCGCGGAGATCAACTTCAACATCGACACTGACGA |
| | | GGACAGTGTGAGCGAGCGGGGCTGTGCGGGGTCATGCAGGCACCCTGTTCCCAGGCAGCTCAGGCCGCGCCCATGGCTCGGTCTGTGG |
| | | TGGGCCTGTGCGGTGGGGCTGGGAGAGGCCCCTCTGTGGAGCTAGGAACAGTCGCTTTTCTTGACCCTCCCCATCATGCCCTCCAGCCCA |
| | | TGGCGCCCACATCCTGAACTAAGCCCCTCTGGGAGCCCTGTGGGGAGAGCGCCTCCTGTCTCCCCCAGACCCTCTGGAAACTGACCTTGG |
| | | CGTTTTACTCTGCAGCCCAGCGCGGCTCTGAGGCCTGCTGCAGCGACCGCATCCAGCACTTTGATGAGAACGAGGACATCTCGGAGGACA |
| | | GCGACACTTGCTGTGCTGCCCAGGTGAAGGCCAGAGCCAGGTGCGGGGCCTGCCCATCCCCCCAAAGCCTCTGCCGAGGAGGTGCAGC |
| | | CCCCAGAACACCCGTCAGATGCCCAGACGCCCTGCTGTTTGTTATGCCGG |
| 176 | chr21: | TTTGGGCCACGAGGCAAGTTCAAAGCGGGAGACTTTTGTTTTATAAAATGATGGTGAGCAGCTCCGGTTTTATGTCAAACATCAGGGTTT |
| | 15649340- | CGTGCAGGATATAAACATTT |
| | 15649450 | |
| 177 | C21orf34 | ATTGCCGTACTTTGCTTCCCTTTGTATGTATTTCTTGTATGCTGCCGAGTCACTGATGGCTAGCTCTGTCTGGCAAGTAATTCAAAAATG |
| | | CTGTTTATGTAGAAAGGAAAGGTAGGGACTTTACCACACTCTGTCATTAAAGGGAGCAATTGAAGAACAAAGGAACTGAGTAAATACCTA |
| | | TATATTGCCTTTTGTGTTGCGAAACACTGTAGCACAAACACATTTGTGTTCAGCCAAATGTTTTACTTCCTTTTGTAATAACGCATATAG |
| | | TAGGTTGTCTCCACATATGTACAAGAATCCATATTTTATTTAAACGTATATAGTCAATTGTTCATATTTATAGGCTGCAAACATTTCTCA |
| | | ATCTCAAAGACTTTTACATATCCACTCCCACACAGCTATTTGTTATTATTTTAAAAGTTCTTAAATTAAAAAAAAAAATAAAATATACTA |
| | | ATATCTCTGTTGGTTGATTTTATTAAGCAACTTAGGATTTCAACACAGTTTAAATCATATTGATGACTCAGATCCTGGCAGGTCTTACAA |
| | | TTCCTGTGAAATGAGAGCACAGCTAATAAAAATATTAAGCAATTACTTTTATTAAAATCATAGGGTTTTTTTCATTATCACATAGAAATG |
| | | ATTGATCTATACAGATTGGTCTCACTCATGTGTCTTTTGGGCTGCTTGGGAGCTTCATGTAGAAGTGGAAAGTCCCCTTTGCTCTTCCTT |
| | | CGACCAAGGTGGGGAAAATGAAGGCATAGAATACAATCTAGGGCTATTAAAGAATTGCTGGCATTACTTCTCTCTATCACGTGTGAGCCT |
| | | GGCTGCCTGCTTCCTGAGGTAGGGGATCCAGGATGAGACTGTGCCGGAGCCTGTTTCCACAACTGCATTTGGAGATCCGTCTTATTGATT |
| | | AGCGGGGGAAAGGGGTGGGGATCAGGAGTGTGAGGTGAGGGGAGGACCAACTGACGACTGGCTCAATGAAGCACAAGACATTTTCTTCCG |
| | | GAAAGATGTCAAACAACTGAGAAACAGCCAGAGAGGAAGTAGAAAGGTGGAAAAATGAGGAGACCCTGGAAGAAATGAAGGCATTTCCTA |
| | | TGAGACAGCCTTGGGGCTTTTTTCTTTTCTTTCTTTTTTTTTGCTTCCATCATCTGACCTGCAAAGGCTAGAGTGACAGCGTCATGCAAA |
| | | TGCTGCAGTCCAGCAGGTCTGGGAGAGGGTGGATGCTAGACTGTGAGTTAATGTTAATGATGAGCGCAGTGAAAATACCAGCCGCTGCCA |
| | | CCCCCTGCTCACAGAAGCGCTCTGAGTCAGCATCAGATGCTTTGCCTCGCCTCTCGCTGTGTATCTGTATGCCTGTGTGCGCGCGCGTGC |
| | | TCGCTCGGGCATCCGTGTCTAGCCGAGGGGAGGGGGTGGCGTGTGAGTGCGTGGAGGGTAAAAGCCAGTCAGTCAGTGAGAAGCAAAGGT |
| | | ACGTTGGAGAGCAACTAAAATCTGACTGATTTCCATCTTTGGAGCATCAGATGTATTCCC |
| 178 | BTG3 | GCAGCCTCCTCCTGAAAAATGTAAGCCATTTCCACTTTGTAAAGCTACGTTTATATTCCACCACGATACGATGGAAAAGAAAACCCAAGG |
| | | CAATTTAATATACGGGTTGGGAAGAAAGTTTTGCTGATGGAACTACATTAGCCTCCACTCCAGCAAAGCAAACAAGGAACCACACTAAAG |
| | | AAATGTACTGAATCTTTTAA |
| 179 | CHODL | TGCCTGAGCGCAGAGCGGCTGCTGCTGCTGTGATCCAGGACCAGGGCGCACCGGCTCAGCCTCTCACTTGTCAGAGGCCGGGGAAGAGA |
| | | AGCAAAGCGCAACGGTGTGGTCCAAGCCGGGGCTTCTGCTTCGCCTCTAGGACATACACGGGACCCCCTAACTTCAGTCCCCCAAACGCG |
| | | CACCCTCGAAGTCTTGAACTCCAGCCCCGCACATCCACGCGCGGCACAGGCGCGGCAGGCGGCAGGTCCCGGCCGAAGGCGATGCGCG |
| | | CAGGGGGTCGGGCAGCTGGGCTCGGGCGGCGGGAGTAGGGCCCGGCAGGGAGGCAGGGAGGCTGCAGAGTCAGAGTCGCGGGCTGC |
| | | GCCCTGGGCAGAGGCCGCCCTCGCTCCACGCAACACCTGCTGCTGCCACCGCGCCGCGATGAGCCGCGTGGTCTCGCTGCTGCTGGGC |
| | | GCCGCGCTGCTCTGCGGCCACGGAGCCTTCTGCCGCCGCGTGGTCAGCGGTGAGTCAGGGGCCGTCTCCCCGAAGAACGAGCGGGGAG |
| | | AGGGGACCACGGGGCGCGGCGGGCAGCCTGTTCTCGGGCGGAGGCTCTCCGGGGCGTTGGAAACCTGCATGGTGTAAGGACCCGGGAG |
| | | GAGGCGGGGAGAAATTGATTGTGCTGTTCTCCTCCCTCTCTTCTCTAACACACACGCAGAAAAGTTTAAATTTTTGTGAAGCGCTTGCTT |
| | | ACGTAGCTGCGGAGCGAGCCTCTGCTTCATTACGAGCGGCATAGCCTTTTTCAGGAGTGATTTCCACTTTCTTTGTGAGAGAGTTGACCA |
| | | CAC |
| 180 | NCAM2 | TTCAATTTACACTCGCACACGCGGGTACGTGGGTGTTCGGGGTAGGGCACTGATCTGGGGAAGGTCTCCCCCCCGCGACCCAACTCATCT |
| | | TTGCACATTTGCAGTCCTCCCTCGGTGCACTCCTGGCGGGGATCTGGCCAGTGCAGCGCACTGGGACCGAGGGCAGAGCCCGCGGAGTG |
| | | AGGCCAGGAGAGACTTCAGGCCTCTAAGGACACAGCTGAGGCTAAGGCTGAGTTGAACGCAGCCCCTCCCGCGGCTCGTCCCCTCTCCA |
| | | GTGTCTCTCCCGTAAGGTGCCGCTCCCAACAGCAATGGGTCGAGATGTAGAGGAAACACTCTGTACGTTATTTTTCCGCCCACCCTTTAG |
| | | CGCCTGAGGAGACAGACAGTGTAGACTTTAGGGTACAATTGCTTCCCCTCTGTCGCGGCGGGGTGGGGAGCGTGGGAAGGGGACAGCCGC |
| | | GCAAGGGGCCAGCCTGCTCCAGGTTTGAGCGAGAGAGGGAGAAGGAGGTCCACGGAGAGACAAGAATCTCCCTCCTCCCACGCCCAAAA |
| | | GGAATAAGCTGCGGGGCACACCGCCCGCCTCCAGATCCCCCATTCACGTTGAGCCGGGGCGCG |
| 181 | chr21: | TCATTATCCGATTGATTTTCCTGGTATCACATCACTTAAGTTTAAGTAGCTCTTATGTTACTTAGTAATGACTGCAAAACACGAGTTGTG |
| | 23574000- | ATGCGGGCAATTTGGATACAACAAAAAGAAGCCATTAAGTTTGTTCGTTAGTTAACAGGTGAAAGCTCTCAAGTTATTAAGGATAAAAAT |
| | 23574600 | GCTAGTATATATATATATGGTTTGGAACTATACTGCGGATTTTGGATCATATCCGCCATGGATAAGGGAGGAATACTATAATCAGGTTTG |
| | | TTTTAAATTCCATGTCTAATGACTTCGTTATCTAGATCACCTGTAGAGCTGTTTTTATTGTAGGAGTTTTCCTTGGTTTTAATCTTTTGA |
| | | TTTGTTTTTCATGTTAATACTGAAATTTTTAAAAATTGCATATTGTACTTCCTATATGAAAATTTTACTATGTATTTTTATTTTTATTTT |
| | | CCTTTTCCTTTAGGAAGAATTAGTTTGTTCCCTGACAGAGTTAGAGTAAGGGCAAATTACTTGTCTCTATAAACAACTCAGATGTTTTGA |
| | | GCCGGTGTTGTAGGGGTTATCTTTTTCTGGTTTTGCATTTTATTATAGGACATAGTGCTT |
| 182 | chr21: | AGAAAGAAGAAATCCGGTAAAAGGATGTGTTATTGAGTTTGCAGTTGGTGTTTGATCTTGCACAGATTTTCTCAGGGGCCTTAAGACCGG |
| | 24366920- | TGCCTTGGAACTGCCATCTGGGCATAGACAGAAGGGAGCATTTATACGCC |
| | 24367060 | |
| 183 | chr21: | CGAAGATGGCGGAGGTGCAGGTCCTGGTGCTCGATGGTCGAGGCCATCTCCTGGTCCGCCTGGCGGCCATCGTGGCTAAACAGGTACTGC |
| | 25656000- | TGGGCCGGAAAGTGGTGGTCGTACGCTGCGAAGGCATCAACATTTCTGGCAATTTCTACAGAAACAAGTTGAAGTACCTGGGTTTCCTCC |
| | 25656900 | GCAAGCGGATGAACACCCACCTTTCCCGAGGTCCCTACCACTTCCGGGCCCCCCAGCCGCATCTTCTGGCGGACCGTGCGAGGTATGCC |
| | | GCCCCACAAGACCAAGCGAGGCCAGGCTTCTCTGGACCGCCTCAAGGTGTTTGACCGCATCCCACCGCCCTACGACAAGAAAAAGCGGAT |
| | | GGTGTTCCTGCTCCCTCAAGGTTGTGCGTCTGAAGCCTACAAGAAAGTTTGCCTATCTGGGGCGCCTGGCTCACGAGGTTGGCTGGAAGT |
| | | ACCAGGCAGTGACAGCCACCCTGGAGGAGAAGAGGAAAGAGAAAGCCAAGATCCACTACCGGAAGAAGAAACAGCTCATGAGGCTACGGA |
| | | AACAGGCCGAGAAGAACATGGAGAAGAAAATTGACAAATACACAGAGGTCCTCAAGACCCACAGACTCCTGGTCTGAGCCCAATAAAGAC |
| | | TGTTAATTCCTCATGCGTGGCCTGCCCTTCCTCCATCGTCGCCCTGGAATGTACGGGACCCAGGGGCAGCAGCAGTCCAGGCGCCACAGG |
| | | CAGCCTCGGACACAGGAAGCTGGGAGCAAGGAAAGGGTCTTAGTCACTGCCTCCCGAAGTTGCTTGAAAGCACTCGGAGAACTGTGCAGG |
| | | TGTCATTTATCTATGACCAATAGGAAGAGCAACCAGTTACTATTAGTGAAAGGGAGCCAGAAGACTGATTGGAGGGCCCTATCTTGTGAG |
| | | C |
| 184 | MIR155HG | GCCTGAAGACCATTTCTTCCTCTCTTAGGGACCTGCTGGTCTCCAGCTGATTCGGTCCAGGAGGAAAAACCTCCCACTTGCTCCTCTCGG |
| | | GCTCCCTGCAAGGAGAGAGTAGAGACACTCCTGCCACCCAGTTGCAAGAAGTCGCCACTTCCCCCTCCAGCCGACTGAAAGTTCGGGCGA |
| | | CGTCTGGGCCGTCATTTGAAGGCGTTTCCTTTTCTTTAAGAACAAAGGTTGGAGCCCAAGCCTTGCGGCGCGGTGCAGGAAAGTACACGG |
| | | CGTGTGTTGAGAGAAAAAAAATACACACACGCAATGACCCACGAGAAAGGGAAAGGGGAAAACACCAACTACCCGGGCGCTGGGCTTTTT |
| | | CGACTTTTCCTTTAAAAAGAAAAAAGTTTTTCAAGCTGTAGGTTCCAAGAACAGGCAGGAGGGGGGAGAAGGGGGGGGGGGTTGCAGAAA |
| | | AGGCGCCTGGTCGGTTATGAGTCACAAGTGAGTTATAAAAGGGTCGCACGTTCGCAGGCGCGGGCTTCCTGTGCGCGGCCGAGCCCGGGC |
| | | CCAGCGCCGCCTGCAGCCTCGGGAAGGGAGCGGATAGCGGAGCCCCGAGCCGCCCGCAGAGCAAGCGCGGGGAACCAAGGAGACGCT |
| | | CCTGGCACTGCAGGTACGCCGACTTCAGTCTCGCGCTCCCGCCCGCCTTTCCTCTCTTGAACGTGGCAGGGACGCCGGGGGACTTCGGT |
| | | GCGAGGGTCACCGCCGGGTTAACTGGCGAGGCAAGGCGGGGGCAGCGCGCACGTGGCCGTGGAGCCCGGCCTGGTCCCGCGCGCGCC |
| | | TGCGGGTGCCCCCTGGGGACTCAGTGGTGTCGCCTCGCCCGGGACCAGAGATTGCGCTGGATGGATTCCCGCGGGCAGAGGCAGGGGG |
| | | AAGGAGGGGTGTTCGAAACCTAATACTTGAGCTTCTTTGCAAAGTTTCCTTGGATGGTTGGGGACGTACCTGTATAATGGCCCTGGACCA |
| | | GCTTCCCTGTTGGAGTGGCCAGAGAAGTGTGTAAAACACACTAGAGGGGCAGGGTGGAAAAAGAGACTGCCTTCAAAACTTGTATCTTTT |
| | | CGATTTCATTTTGAAAAATAACTACAAATCTATTTTAATTTTACAAAGTTAGACTCATAGCATTTTAGATATCAATGTCTTCATTTAACA |
| | | GAAGTGAAGATGGAGCAAACGCTCAATCAGCGTCTGTATTTATTCGCTCCTGTTGTGCCAGGGTGCGTTTTTGCCGAGCGGTTGCCTTTC |
| | | TTTACTCACAAAACCCCCTTGATGTCTGTCCTCCACGTTTTACGAGGGAGAGCCGGATCTTTTGAAGTTTGTATCATCTAAAGCAGGTAT |
| | | ATTGGGATGACTATGGATAGAATTTAACCTGAAAACACTGAAGTTGACAGCTGACAAAG |
| 185 | CYYR1 | CATAACAAGAGTCATTCTAATGTGATTATAAAGGACCCGAAGCTTTGCTTTTAAAATTCAATACTTAGGTAGAAAGAAAATGATAACTTT |
| | | TTCCCTTTGATTTTTATTCACTATTTTTATAACACTAGCAGCCCTGAGACACCGGATTGGAAATATCTATGCCTCTTGATGTTACCTGGG |
| | | CACCACTGCATCACAGTCCT |
| 186 | chr21: | AATAGTAATTGCCAACAGTCAAGATATGTACTACCACCAAATTCCGTGTTATTTGTGATCAAAAGATATACACAGATACTTGAAAACTGA |
| | 26938800- | TTTCTACGTTGCATATGGGAAAAATACCTCATTTTTCTCAGCTGTCCATTATTTTTGAGATATTATGTGCAGTGATAGTAAGAACAAGCA |
| | 26939200 | GATTTGGAACACATCAGCAATAATTTTTTCAATCAGAGTCCTGCCAAAATGAAAGAATTTGACAGTATCCGGCACCCTGTACTCATGCTT |
| | | GGCTTCTGTAGAAACTGTGGCTTGCAAAAGGGCAGCTGGGTACTGTGTTTTGGTACCTCATTCTTTAAACGTATAATGGGAATCTGGTTG |
| | | GTTCAGGAAAACCCTTGCCTACTTATTATTACTCTGTTTT |
| 187 | GRIK1 | GGCCCATACTTAATGTATTTTTAAACGTTTTAACATTTACTAATATAGAACCTTCTATTGCCTATTTCCTTCTGGTTTATTCCCTTTCCT |
| | | TCTGTCATTGAAGAAATGGTTCTAGTGGTAGAAATACTCCACGATTGAGAAGAATGTGGGAAGAAAGGAGGGCTGGTGGGTAAGAATTGC |
| | | TCATGATGTCTCCCTCTGAATTCTGTGCTCTCACAATGACACTCCAATGTGTGGTTTGACGCCTGGAAGA |
| 188 | chr21: | TGCTTCAACCGGAAATGTGGTTGAATTACCCTTACAGTGAACCTGATCAGTGGTAACAGGAGATGCTAGAACAGGAAAAGACAAGTTTCC |
| | 30741350- | CCTTTCCTCCCTATCCCATCAATTACTTTGAGGTGTATTTTTTCTTTGCAACCCCTCCAGAGAAGTCGGCAATGTTTAACGAGCATGCCT |
| | 30741600 | GCCAAGTGGCTTGCCTTATACCTCATTATGAAGTGATACTCAGGGCCACTAACACATCGCACAGCATTGC |
| 189 | TIAM1 | TATGATTCCCTCGATTTCCCTCAATCTTAACCATTGTGGATCACAGCAGGAGGGCCAGAAAGTGAGCTTCAGCCTGGCACCGGGACCTCA |
| | | GCCTCTCCCTTAAACTTTCCCTAATCCTCGGAGCTAGTGTTACTCAAGTGACTCCACAGTGTTGCCCGATCCCTTCAGACATGGCCTTGA |
| | | TGATCTCCAAAACTCATGCTACCTTTGCCAGCCTAAAGCATCCACTCTGTGCCCCAAAACGTGAATGTCAAATACCCTTCAAGGCAGAAG |
| | | GCTATTTCTATTTTTGTTTGTTTCTGTTTAAGGCAACAATCACCAACATTTGGTACACATGAGCCATCCTGTGAAACATCAAGGCGCTTC |
| | | GTTGGCAGCAAGTCAACTTCGGTTTCAGAAGAAAGCTGCACTATTTCCTGAGGTTAGAGGTTTAAACCAAAACAAGACAACCACATTTTA |
| | | ACCCCAAATCTGCCGACTGAGGGTAACCATGATCCTTCCTTCACAGCACC |
| 190 | TIAM1 | TACTAAATCAACCCAAACCCGAGAACCCGGTCATGGAGAAATAAATGATAGTAATCTATGCTGTTCATCTGTTCCATCACTCACTCACTC |
| | | TCTTGCTGAACAAGAAAGGGCCACCCATGTAGCAAACCACATGTAAAGAGCCGGGAAGAC |
| 191 | TIAM1 | TATTATTTTGTTCAAAGTAGACGGGTATACTAACATCTGTGGGCAAGTTTACCACACGCCACTTAAAACAGGCTAACAGGGTCATATGCC |
| | | AAAACGTTCAGGTTTGCATTTTTGAAAAGCTCAGAGATCTGACAGATGTGTTCCGGCCGCGATTTAACATGCGGCTCCAGTGAGAAGGAA |
| | | GCAGATATGACAAATGGTTCACTTATTTCAGAACTAAAACCCCAGAGGAGCAGCCTGAGCCAAAAAGGGAAGTGATCAATGGAAAAGACG |
| | | GTCGAATCTGCTCACAGGCAAGGCAAGGGG |
| 192 | SOD1 | AAGACCTGGAGTTTCCATTACACCGAATTGGCACTTAATAACTGTTGTCGGAGCATTTCTTAAGCCACATTTTCGTAAAGTGGCTTTAAA |
| | | ATTGCTCTGCCAGTAGGCAGGTTGCTAAGATGGTCAGAGACAAACTTCTGAACGACTCTTGTAAAATATACAGAAATATTTTCAGAACTT |
| | | TTATCAGTAAAATTACAAAACGTGTTGCAAGGAAGGTGCTTGTGATAACACTGTCCCCAGAACCTTAGTGAAGTTACCAACTGGTGGAAA |
| | | ATTTTCTCTTGCACTCGGCTTAAAAATCAT |
| 193 | HUNK | GCAGGGGTGACTGGTCCTCTCTCTCTGCACCTCGCAGGATTTCTCTGGAAGATCTGAGCCCGAGCGTCGTGCTGCACATGACCGAGAAGC |
| | | TGGGTTACAAGAACAGCGACGTGATCAACACTGTGCTCTCCAACCGCGCCTGCCACATCCTGGCCATCTACTTCCTCTTAAACAAGAAAC |
| | | TGGAGCGCTATTTGTCAGGGGTAAGTGCGACCCTAGAGGCGATCGTCTCTGCTGTCTGTGGAAAAAAGAGCTCCTACACCCAAAGTGCTT |
| | | CTCAGTTGCTGACACTTGATCCAAGCTGCTAATTTAATCTAATGTGAGGCTGAGTTTTCTGAATGTGGGATAAAGTCGTAGCTAAACCTG |
| | | CTTCTCAGGGAGTGCCTTTTATCTGCAATGTTTTTCAAAT |
| 194 | chr21: | AAGTAACGGGATCAAATTAATTATTATTTTGGTGGCCGCCTCTCTTCTCCACCCCAAGCCAGGCAAGACTCACCCTCGGCCCTGCCCGCC |
| | 33272200- | CCAGCATTTCAAATGGAATACCTAGGTGGCCCAGGGGGACCCCTGACCCCTATATCCTGTTTCTTTCTGCCTGCTTTGCTACTTTTCTCC |
| | 33273300 | TTGATAAAAGGAGAGAGTGAGAGATAATTAACAAAAAACATGGCCCCAGGACAATGAAACAACTGGCCTTGGCCGGCCAGAAATGTATCC |
| | | TGGTTTTCTAGGTGAACTTTCTCCCATCAATCTTTCCTTTAACCTCTCTGTTAGTGGAAGCAATAGGAACACCCCTCCCCTCCCCTGAGC |
| | | AAATGCTTTCTTTTGACTGGAAACAAAACAGGGGCTCGGCGAAGGCTGAGGTGAAATCTGGGTGGCATGGGCGCCGCACAATGGGGCCGC |
| | | TGTTCCCCGGCCCGGGCTTGTGTTTTACAACAGGGGAGGGGCGGGCGTGAATGGTCTGATGATTGGAACAATCCCCCCGATTCAGGCCTA |
| | | CAAACGCATCTTCTGTTCCACACCGAGGGGACAGAAAGGAGAAAAGTGACAAAGAACGCGGGGCGGGGGGAATTAAAACAAAATGCGCTC |
| | | GACTAAAAAATCTCTCATATCCTGCATATTCCAGAAAGCGGCTCTATGGAGAGAGCCTTCAGGAGGCCTCAGCCATATCTGAATGGCTTT |
| | | CTCTGGCCTCTGATTTATTGATGAAGCTGAAGCGACTTGCTGGAGAAAGGCCTGGAGCCTTCTTTGTCTCCGAGATGAAGTACAATAGGC |
| | | CACAGGGCGGAGATCTCTTGTGATGCTCTCGGGTCCTGCCTTTCTCTTGCCCTCTCCTCCCTGCAAATACCAGCAGCGGTGACAAACGAT |
| | | TGGTGGTGTGCCTGGGAGAGCCGGTGACAAGACTGGGCCACTTGAGGTCTCCTTAAGAGGGTATTATGGCCAGGGCGACGTTTGTGCTGT |
| | | GAAGATGGCACACTCCATTTTGTCAATGGCTCTCATCGGCCCAGATAATCGCCCCCTGCCTGCCTGTCAGGGGCGCAGCCGGCCGATTCA |
| | | TGGCGCCCTCGGAGAAAGTA |
| 195 | OLIG2 | GTCTTTCCCGCCCCCTTGTCTAAACTCAAAACCGAGTCCGGGCGCGCCTTGCAGGGCGCCCGAGCTCTGCAGCGGCGTTGCGGGCTGAA |
| | | CCCATCCGGCACAAACTGCGGGCCACTGGCCCCTCACACCTGGGAGTTTGCGGCGCTGGCCTGCAGCCCGGGGCCCACGTGGCGGAAG |
| | | CTTTCCCGGGCGCGCGCTGCGCAGCCCCGCGGGGCCGGGGAGACACCGCTCGGGAGTCCTCCGCTCGGCTGCAGAATCTTTATCAGCT |
| | | GCACTTTACCGCAGCCCTGGCTAGGACGCTAGGCGGTGGAGCGCCCTATCCAGGTGCGCCGCCGCACCATGGATCACCGCGCCCGGTCC |
| | | CGCAGTCCCGCCATGGCCTGGGGAGGCCCGAAGCCCGGGGACAGTGGCCGGCCCATCTCCGGCTCCGCGGACCCCCGGCTCAGGCGG |
| | | GAGGGCAGGCGGGTCCCTGCAGGCCCCCAGGGAGCCCGGGAGCCTCTCTCTGGCGTCATTCAGTCCCGGGGCAACCTGAAGCGCGGTA |
| | | GATATTGGAGAGGGGGCGTCTGTTGGGGGGACCTGGCGTCATTACTGATGGCTAGCAGGGAGGAGGGAACGGGTTGTCACCTCGGCCTCA |
| | | TAAGGCCGTGAGTGAGTAGTCCAGGGCCTCTTCAGGCATTTTTGAAACTGGATTAACTAGGGGGGAAATTGTAGCACTGAAGCCACCGTG |
| | | ACTGTCTTTTGCGCTGTGTGGAAACTCCGGTAAAACTCTTTGGGCAACAGTCTTATCACCAGCTCTTCAACGTGTGCAGCCCTTCTGGTC |
| | | CTGTCCCTGTTCTGGGCCCCAGGAATGCAAAGCAGGTCCAGGCACTGTGAAGACCCTGGCGGTGGAGGAAGAGGCTTCCCGGCTGTGGAG |
| | | GAAGCCAGACCCTTACAACACAAGACGAGAACCAGACCTGCGTGGGGGAGCTCTGGATGCTACAGGGGCTCAAGGAGGGGTGGAGGGGCC |
| | | TTCCCAGGCCAACCCCTGAACGGCTTGGACAAGATGCTCAGATGGACGGGAGGAACGGCGTGTGGGATGGGGGAGCTGGAGGCGGGTG |
| | | GGTGGGGGGGGGAGGATGGGGAAAGCGCTGGCCCACCCAGTGTGGGAGGGGTAGAGGAAAAGCCCGCAGGGGCCAGGTTGGGACCCCG |
| | | TAGGCCGGGTTAGAGGGCTTGGACTTGATCCTGACAGGCGACAGGGAGACATATTGCTACTTATTATGTGCACAGTGGCCAGATCTCTAA |
| | | AGAAAACACCATCCCCCACCCCCACCCCCCATATAGTAAACCAGGTGGTCCGCCCAGTGCTCCCAGGGAGGTGATGGGAAATCCCACTCC |
| | | ATACCCTGCGGTGAGGGGTTCCATGCCCTCCACGTGTGCAACTACTCCGGGCCCAGGGAAACACTGGGCCCCATCCGGTAACCCCCGGC |
| | | CCAGTCGGGTTTCCCAGTTCACATTATAACCAAACGGTCTTGCCAGCTAGACAGACAGACACCCCTGACCTGTTTACCCTGATCCTCTGC |
| | | TCTCAGGATTAATCACAACTTGTCGAAGGGGGTGGCTTCCAGTGGGGTGGACCGCTCTGTCAATGCCAGCGTGTGTCTAGCATCTCCTGG |
| | | GGTGGGGGTGTGGGGAAGGGAGGTGTAGGATGAAGCCCTAGAAGCCTCAGGCAATTGTGATCCGGTGGGCTGGATACTGAAGCCCACCCC |
| | | TGCCTTGACCTCAATTTTCAGTATCTTCATCTGTAAAATGGGAACAACCTGCCTTCCTCCTAGCCCTAAAGGGGCTGCTGTCAAGATTGG |
| | | CTGAGATAGCTGTTTGCAAGCTGAGCTCAATGAAAGTTCATTGTGTCCCCCTCAGTCCTATCCCAATATCGTCTCACTGCAAAGGTGGGG |
| | | GGCAGCTTAACTTCAAGGGCACTTCAAGGATAGCCAGGTGGCTGTCAGCCCAGCTTTCCAGGATGGGAGCAGGATCTTGACAGAAGGGTT |
| | | GACTGGGAGGGGCAGTTGCTGGTTTGGGCTTCGTTAGGTTGCATTTTTGTTTGTTGTCCTTTCATTTCCCTGGGGCAGCACCCCTTCCTG |
| | | CAAGCTCCAGGCCTTCCTCTGGAATGCTCCTAGAGCCCAACCTCTGCTGGTGCCTGAGCTTAAGCCAGGCCAGCTAAGGGGATCCTGGAT |
| | | TCACACGGCCTCACAGTCACTCAGATTGTTAGCAGAAGACAAAAATTACAAGGGGAGGGCGTCATGTGATTCTTACACACCCTCCAAATC |
| | | CAGCAGACACCTTGGAAGCCACAGGTAGCTTCAAGAAACCCATTTTACGGATGAGAACCTGAGATGGAGAAAGGACAACTGGAGATCTCT |
| | | GAGTCTCTGAGCCCACACTCCCTACCTCCCTGCACCTCCAGGCACTCTGCTGGCAGGATCTTGGGCAAATGCCCACAGCTCTCTGAGAGT |
| | | TCAGTTTTCCGTCTGTAAAATGGGAGTCATACCTTCCTCCTATGGCCGGTGAGAGACTAAATTAAACTATGTCTGTCAAGACACCTGAAA |
| | | CTCCTGGCACAATTTAGGTTGCCTTCAAGTGGTCACAGTTGTCATTAGGTGGAAGTCAACACCCCAATCATTGTAAAGGTGCCCATATAC |
| | | CCCAAGATCCAGATTACAGCTCTCACAGTTTATTATATACAGCGAAAAAACACATAACACACCTTTGCCCACATTTACATGTATTTTACG |
| | | GACCATGTTTCACATCAGTCCGCATGCACATCTGCACGTGTGTGCATTCGGCAGTATTTACCAAGCACCTGCCAAGTGCCAGGGCCTGTC |
| | | CTCCGCACCCGGCGTGAACTGTCCTGGACCAGTCCCGGGAGCCGCGGTTCTGACCAGCCGTGCTGACCCTGGACGACTCCATGAGCTGTT |
| | | TTGTGAGAAAGACACGCCATTTGTTTGCAGAGTTCTGACTTCTGAGGGGTCATGTAGCACATGTTTGGTAGCCAAACGCTGTCATTCACG |
| | | ACCAGGAGCGATGGCTGCAATGCCTTTTTCTTTGCTTTGCTTTCCGGTGCCGGGAGCCTTGCCTCCCGCCGCCACCCCTGGTCAGCTCTG |
| | | CGCAAGAACGTCGTTCTGTTTGGCAGCCAGGCCGAGACGCAGCCTGAATGTGAGCAGGAACTCGGAGAAGGGAAGGGAGAGAATCAGAAA |
| | | GAAGGCCCGGGAGGGACCCGGGAAGCAGTGGGAGGTCTGCGCCCTGGAGCCCCGCGAGAGCCCGCCGGTTTGGCACGGGCTCCTCCCGGG |
| | | CCGCCCGGCGGTCCAACAAAGGCCGGCCCCGACACGCACCCGGTCTTTTGTGGGAGAGAAACACAAAGAAGAGGGAAAAACACGGAGGAG |
| | | GCCAACAGCACCAGGACGCGGGGGCCAACCAGGAACTCCCGGAGCCGGGGCCCATTAGCCTCTGCAAATGAGCACTCCATTCCCCAGGAA |
| | | GGGGCCCCAGCTGCGCGCGCTGGTGGGAACCGCAGTGCCTGGGACCCGCCCAGGTCGCCCACCCCGGGCGCCGGGCGCAGGACCCGGACA |
| | | AGTCCTGGGGACGCCTCCAGGACGCACCAGGGCAAGCTTGGGCACCGGGATCTAATTTCTAGTTATTCCTGGGACGGGGTGGGGAGGCAT |
| | | AGGAGACACACCGAGAGGTACTCAGCATCCGATTGGCACCAGGGCCAAGGGAGCCCAGGGGCGACACAGACCTCCCCGACCTCCCAAGCT |
| | | ACTCCGGCGACGGGAGGATGTTGAGGGAAGCCTGCCAGGTGAAGAAGGGGCCAGCAGCAGCACAGAGCTTCCGACTTTGCCTTCCAGGCT |
| | | CTAGACTCGCGCCATGCCAAGACGGGCCCCTCGACTTTCACCCCTGACTCCCAACTCCAGCCACTGGACCGAGCGCGCAAAGAACCTGAG |
| | | ACCGCTTGCTCTCACCGCCGCAAGTCGGTCGCAGGACAGACACCAGTGGGCAGCAACAAAAAAAGAAACCGGGTTCCGGGACACGTGCCG |
| | | GCGGCTGGACTAACCTCAGCGGCTGCAACCAAGGAGCGCGCACGTTGCGCCTGCTGGTGTTTATTAGCTACACTGGCAGGCGCACAACTC |
| | | CGCGCCCCGACTGGTGGCCCCACAGCGCGCACCACACATGGCCTCGCTGCTGTTGGCGGGGTAGGCCCGAAGGAGGCATCTACAAATGCC |
| | | CGAGCCCTTTCTGATCCCCACCCCCCCGCTCCCTGCGTCGTCCGAGTGACAGATTCTACTAATTGAACGGTTATGGGTCATCCTTGTAAC |
| | | CGTTGGACGACATAACACCACGCTTCAGTTCTTCATGTTTTAAATACATATTTAACGGATGGCTGCAGAGCCAGCTGGGAAACACGCGGA |
| | | TTGAAAAATAATGCTCCAGAAGGCACGAGACTGGGGCGAAGGCGAGAGCGGGCTGGGCTTCTAGCGGAGACCGCAGAGGGAGACATATCT |
| | | CAGAACTAGGGGCAATAACGTGGGTTTCTCTTTGTATTTGTTTATTTTGTAACTTTGCTACTTGAAGACCAATTATTTACTATGCTAATT |
| | | TGTTTGCTTGTTTTTAAAACCGTACTTGCACAGTAAAAGTTCCCCAACAACGGAAGTAACCCGACGTTCCTCACACTCCCTAGGAGACTG |
| | | TGTGCGTGTGTGCCCGCGCGTGCGCTCACAGTGTCAAGTGCTAGCATCCGAGATCTGCAGAAACAAATGTCTGAATTCGAAATGTATGGG |
| | | TGTGAGAAATTCAGCTCGGGGAAGAGATTAGGGACTGGGGGAGACAGGTGGCTGCCTGTACTATAAGGAACCGCCAACGCCAGCATCTGT |
| | | AGTCCAAGCAGGGCTGCTCTGTAAAGGCTTAGCAATTTTTTCTGTAGGCTTGCTGCACACGGTCTCTGGCTTTTCCCATCTGTAAAATGG |
| | | GTGAATGCATCCGTACCTCAGCTACCTCCGTGAGGTGCTTCTCCAGTTCGGGCTTAATTCCTCATCGTCAAGAGTTTTCAGGTTTCAGAG |
| | | CCAGCCTGCAATCGGTAAAACATGTCCCAACGCGGTCGCGAGTGGTTCCATCTCGCTGTCTGGCCCACAGCGTGGAGAAGCCTTGCCCAG |
| | | GCCTGAAACTTCTCTTTGCAGTTCCAGAAAGCAGGCGACTGGGACGGAAGGCTCTTTGCTAACCTTTTACAGCGGAGCCCTGCTTGGACT |
| | | ACAGATGCCAGCGTTGCCCCTGCCCCAAGGCGTGTGGTGATCACAAAGACGACACTGAAAATACTTACTATCATCCGGCTCCCCTGCTAA |
| | | TAAATGGAGGGGTGTTTAACTACAGGCACGACCCTGCCCTTGTGCTAGCGCGGTTACCGTGCGGAAATAACTCGTCCCTGTACCCACACC |
| | | ATCCTCAACCTAAAGGAGAGTTGTGAATTCTTTCAAAACACTCTTCTGGAGTCCGTCCCCTCCCTCCTTGCCCGCCCTCTACCCCTCAAG |
| | | TCCCTGCCCCCAGCTGGGGGCGCTACCGGCTGCCGTCGGAGCTGCAGCCACGGCCATCTCCTAGACGCGCGAGTAGAGCACCAAGATAGT |
| | | GGGGACTTTGTGCCTGGGCATCGTTTACATTTGGGGCGCCAAATGCCCACGTGTTGATGAAACCAGTGAGATGGGAACAGGCGGCGGGAA |
| | | ACCAGACAGAGGAAGAGCTAGGGAGGAGACCCCAGCCCCGGATCCTGGGTCGCCAGGGTTTTCCGCGCGCATCCCAAAAGGTGCGGCTGC |
| | | GTGGGGCATCAGGTTAGTTTGTTAGACTCTGCAGAGTCTCCAAACCATCCCATCCCCCAACCTGACTCTGTGGTGGCCGTATTTTTTACA |
| | | GAAATTTGACCACGTTCCCTTTCTCCCTTGGTCCCAAGCGCGCTCAGCCCTCCCTCCATCCCCCTTGAGCCGCCCTTCTCCTCCCCCTCG |
| | | CCTCCTCGGGTCCCTCCTCCAGTCCCTCCCCAAGAATCTCCCGGCCACGGGCGCCCATTGGTTGTGCGCAGGGAGGAGGCGTGTGCCCGG |
| | | CCTGGCGAGTTTCATTGAGCGGAATTAGCCCGGATGACATCAGCTTCCCAGCCCCCCGGCGGGCCCAGCTCATTGGCGAGGCAGCCCCTC |
| | | CAGGACACGCACATTGTTCCCCGCCCCCGCCCCCGCCACCGCTGCCGCCGTCGCCGCTGCCACCGGGCTATAAAAACCGGCCGAGCCCCT |
| | | AAAGGTGCGGATGCTTATTATAGATCGACGCGACACCAGCGCCCGGTGCCAGGTTCTCCCCTGAGGCTTTTCGGAGCGAGCTCCTCAAAT |
| | | CGCATCCAGAGTAAGTGTCCCCGCCCCACAGCAGCCGCAGCCTAGATCCCAGGGACAGACTCTCCTCAACTCGGCTGTGACCCAGAATGC |
| | | TCCGATACAGGGGGTCTGGATCCCTACTCTGCGGGCCATTTCTCCAGAGCGACTTTGCTCTTCTGTCCTCCCCACACTCACCGCTGCATC |
| | | TCCCTCACCAAAAGCGAGAAGTCGGAGCGACAACAGCTCTTTCTGCCCAAGCCCCAGTCAGCTGGTGAGCTCCCCGTGGTCTCCAGATGC |
| | | AGCACATGGACTCTGGGCCCCGCGCCGGCTCTGGGTGCATGTGCGTGTGCGTGTGTTTGCTGCGTGGTGTCGATGGAGATAAGGTGGATC |
| | | CGTTTGAGGAACCAAATCATTAGTTCTCTATCTAGATCTCCATTCTCCCCAAAGAAAGGCCCTCACTTCCCACTCGTTTATTCCAGCCCG |
| | | GGGGCTCAGTTTTCCCACACCTAACTGAAAGCCCGAAGCCTCTAGAATGCCACCCGCACCCCGAGGGTCACCAACGCTCCCTGAAATAAC |
| | | CTGTTGCATGAGAGCAGAGGGGAGATAGAGAGAGCTTAATTATAGGTACCCGCGTGCAGCTAAAAGGAGGGCCAGAGATAGTAGCGAGGG |
| | | GGACGAGGAGCCACGGGCCACCTGTGCCGGGACCCCGCGCTGTGGTACTGCGGTGCAGGCGGGAGCAGCTTTTCTGTCTCTCACTGACTC |
| | | ACTCTCTCTCTCTCTCCCTCTCTCTCTCTCTCATTCTCTCTCTTTTCTCCTCCTCTCCTGGAAGTTTTCGGGTCCGAGGGAAGGAGGACC |
| | | CTGCGAAAGCTGCGACGACTATCTTCCCCTGGGGCCATGGACTCGGACGCCAGCCTGGTGTCCAGCCGCCCGTCGTCGCCAGAGCCCGAT |
| | | GACCTTTTTCTGCCGGCCCGGAGTAAGGGCAGCAGCGGCAGCGCCTTCACTGGGGGCACCGTGTCCTCGTCCACCCCGAGTGACTGCCCG |
| | | CCGGAGCTGAGCGCCGAGCTGCGCGGCGCTATGGGCTCTGCGGGCGCGCATCCTGGGGACAAGCTAGGAGGCAGTGGCTTCAAGTCATCC |
| | | TCGTCCAGCACCTCGTCGTCTACGTCGTCGGCGGCTGCGTCGTCCACCAAGAAGGACAAGAAGCAAATGACAGAGCCGGAGCTGCAGCAG |
| | | CTGCGTCTCAAGATCAACAGCCGCGAGCGCAAGCGCATGCACGACCTCAACATCGCCATGGATGGCCTCCGCGAGGTCATGCCGTACGCA |
| | | CACGGCCCTTCGGTGCGCAAGCTTTCCAAGATCGCCACGCTGCTGCTGGCGCGCAACTACATCCTCATGCTCACCAACTCGCTGGAGGAG |
| | | ATGAAGCGACTGGTGAGCGAGATCTACGGGGGCCACCACGCTGGCTTCCACCCGTCGGCCTGCGGCGGCCTGGCGCACTCCGCGCCCCTG |
| | | CCCGCCGCCACCGCGCACCCGGCAGCAGCAGCGCACGCCGCACATCACCCCGCGGTGCACCACCCCATCCTGCCGCCCGCCGCCGCAGCG |
| | | GCTGCTGCCGCCGCTGCAGCCGCGGCTGTGTCCAGCGCCTCTCTGCCCGGATCCGGGCTGCCGTCGGTCGGCTCCATCCGTCCACCGCAC |
| | | GGCCTACTCAAGTCTCCGTCTGCTGCCGCGGCCGCCCCGCTGGGGGGCGGGGGCGGCGGCAGTGGGGCGAGCGGGGGCTTCCAGCACTGG |
| | | GGCGGCATGCCCTGCCCCTGCAGCATGTGCCAGGTGCCGCCGCCGCACCACCACGTGTCGGCTATGGGCGCCGGCAGCCTGCCGCGCCTC |
| | | ACCTCCGACGCCAAGTGAGCCGACTGGCGCCGGCGCGTTCTGGCGACAGGGGAGCCAGGGGCCGCGGGGAAGCGAGGACTGGCCTGCGCT |
| | | GGGCTCGGGAGCTCTGTCGCGAGGAGGGGCGCAGGACCATGGACTGGGGGTGGGGCATGGTGGGGATTCCAGCATCTGCGAACCCAAGCA |
| | | ATGGGGGCGCCCACAGAGCAGTGGGGAGTGAGGGGATGTTCTCTCCGGGACCTGATCGAGCGCTGTCTGGCTTTAACCTGAGCTGGTCCA |
| | | GTAGACATCGTTTTATGAAAAGGTACCGCTGTGTGCATTCCTCACTAGAACTCATCCGACCCCCGACCCCCACCTCCGGGAAAAGATTCT |
| | | AAAAACTTCTTTCCCTGAGAGCGTGGCCTGACTTGCAGACTCGGCTTGGGCAGCACTTCGGGGGGGGAGGGGGTGTTATGGGAGGGGGAC |
| | | ACATTGGGGCCTTGCTCCTCTTCCTCCTTTCTTGGCGGGTGGGAGACTCCGGGTAGCCGCACTGCAGAAGCAACAGCCCGACCGCGCCCT |
| | | CCAGGGTCGTCCCTGGCCCAAGGCCAGGGGCCACAAGTTAGTTGGAAGCCGGCGTTCGGTATCAGAAGCGCTGATGGTCATATCCAATCT |
| | | CAATATCTGGGTCAATCCACACCCTCTTAGAACTGTGGCCGTTCCTCCCTGTCTCTCGTTGATTTGGGAGAATATGGTTTTCTAATAAAT |
| | | CTGTGGATGTTCCTTCTTCAACAGTATGAGCAAGTTTATAGACATTCAGAGTAGAACCACTTGTGGATTGGAATAACCCAAAACTGCCGA |
| | | TTTCAGGGGCGGGTGCATTGTAGTTATTATTTTAAAATAGAAACTACCCCACCGACTCATCTTTCCTTCTCTAAGCACAAAGTGATTTGG |
| | | TTATTTTGGTACCTGAGAACGTAACAGAATTAAAAGGCAGTTGCTGTGGAAACAGTTTGGGTTATTTGGGGGTTCTGTTGGCTTTTTAAA |
| | | ATTTTCTTTTTTGGATGTGTAAATTTATCAATGATGAGGTAAGTGCGCAATGCTAAGCTGTTTGCTCACGTGACTGCCAGCCCCATCGGA |
| | | GTCTAAGCCGGCTTTCCTCTATTTTGGTTTATTTTTGCCACGTTTAACACAAATGGTAAACTCCTCCACGTGCTTCCTGCGTTCCGTGCA |
| | | AGCCGCCTCGGCGCTGCCTGCGTTGCAAACTGGGCTTTGTAGCGTCTGCCGTGTAACACCCTTCCTCTGATCGCACCGCCCCTCGCAGAG |
| | | AGTGTATCATCTGTTTTATTTTTGTAAAAACAAAGTGCTAAATAATATTTATTACTTGTTTGGTTGCAAAAACGGAATAAATGACTGAGT |
| | | GTTGAGATTTTAAATAAAATTTAAAGTAAAGTCGGGGGATTTCCATCCGTGTGCCACCCCGAAAAGGGGTTCAGGACGCGATACCTTGGG |
| | | ACCGGATTTGGGGATCGTTCCCCCAGTTTGGCACTAGAGACACACATGCATTATCTTTCAAACATGTTCCGGGCAAATCCTCCGGGTCTT |
| | | TTTCACAACTTGCTTGTCCTTATTTTTATTTTCTGACGCCTAACCCGGAACTGCCTTTCTCTTCAGTTGAGTATTGAGCTCCTTTATAAG |
| | | CAGACATTTCCTTCCCGGAGCATCGGACTTTGGGACTTGCAGGGTGAGGGCTGCGCCTTTGGCTGGGGGTCTGGGCTCTCAGGAGTCCTC |
| | | TACTGCTCGATTTTTAGATTTTTATTTCCTTTCTGCTCAGAGGCGGTCTCCCGTCACCACCTTCCCCCTGCGGGTTTCCTTGGCTTCAGC |
| | | TGCGGACCTGGATTCTGCGGAGCCGTAGCGTTCCCAGCAAAGCGCTTGGGGAGTGCTTGGTGCAGAATCTACTAACCCTTCCATTCCTTT |
| | | TCAGCCATCTCCACTACCCTCCCCCAGCGGCCACCCCCGCCTTGAGCTGCAAAGGATCAGGTGCTCCGCACCTCTGGAGGAGCACTGGCA |
| | | GCGCTTTGGCCTCTGTGCTCTTTCCT |
| 196 | OLIG2 | CCGGCACGGCCCGCATCCGCCAGGATTGAAGCAGCTGGCTTGGACGCGCGCAGTTTTCCTTTGGCGACATTGCAGCGTCGGTGCGGCCA |
| | | CAATCCGTCCACTGGTTGTGGGAACGGTTGGAGGTCCCCCAAGAAGGAGACACGCAGAGCTCTCCAGAACCGCCTACATGCGCATGGGG |
| | | CCCAAACAGCCTCCCAAGGAGCACCCAGGTCCATGCACCCGAGCCCAAAATCACAGACCCGCTACGGGCTTTTGCACATCAGCTCCAAAC |
| | | ACCTGAGTCCACGTGCACAGGCTCTCGCACAGGGGACTCACGCACCTGAGTTCGCGCTCACAGATCCACGCACACCGGTGCTTGCACACG |
| | | CAAGGGCCTAGAACTGCAAAGCAGCGGCCTCTCTGGACCGCCTCCCTCCGGCCCTCCTGAGCCCTACTGAGCCCTGCTGAGTCCTGGAG |
| | | GCCCTGTGACCCGGTGTCCTTGGACCGCAAGCATCCTGGTTTACCATCCCTAC |
| 197 | RUNX1 | GGACGCGGCCCGCTCTAGAGGCAAGTTCTGGGCAAGGGAAACCTTTTCGCCTGGTCTCCAATGCATTTCCCCGAGATCCCACCCAGGGCT |
| | | CCTGGGGCCACCCCCACGTGCATCCCCCGGAACCCCCGAGATGCGGGAGGGAGCACGAGGGTGTGGCGGCTCCAAAAGTAGGCTTTTGA |
| | | CTCCAGGGGAAATAGCAGACTCGGGTGATTTGCCCCTCGGAAAGGTCCAGGGAGGCTCCTCTGGGTCTCGGGCCGCTTGCCTAAAACCCT |
| | | AAACCCCGCGACGGGGGCTGCGAGTCGGACTCGGGCTGCGGTCTCCCAGGAGGGAGTCAAGTTCCTTTATCGAGTAAGGAAAGTTGGTC |
| | | CCAGCCTTGCATGCACCGAGTTTAGCCGTCAGAGGCAGCGTCGTGGGAGCTGCTCAGCTAGGAGTTTCAACCGATAAACCCCGAGTTTGA |
| | | AGCCCGACAAAAAGCTGATAGCAATCACAGCTTTTGCTCCTTGACTCGATGGGATCGCGGGACATTTGGGTTTCCCCGGAGCGGCGCAGG |
| | | CTGTTAACTGCGCAGCGCGGTGCCCTCTTGAAAAGAAGAAACAGACCAACCTCTGCCCTTCCTTACTGAGGATCTAAAATGAATGGAAAG |
| | | AGGCAGGGGCTCCGGGGAAAGGGAACCCCTTAGTCGGCCGGGCATTTTACGGAGCCTGCACTTTCAAGGACAGCCACAGCGTGTACGAAG |
| | | TGAGGAATTCCTTTCCACCAAGAGCGCTCATTTTAGCGACAATACAGAATTCCCCTTCCTTTGCCTAAGGGAGAAAGGAAAGGAAACATT |
| | | ACCAGGTTCATTCCCAGTGTTTCCCTGGAGTAATGCTAGAATTTACTTTTGTCATAATGCAAAATTAAAAAAAAAAAAAATACAACGAAG |
| | | CGATACGTTGGGCGGATGCTACGTGACAGATTTTTCCAAATTTTGTTGCGGGGAGAGGGAGGGAGGAGAATTGAAAACGGCTCACAACAG |
| | | GAATGAAATGTA |
| 198 | RUNX1 | TTTTTAATGCTCAGAGAAGTTCGTATTACTGATTCGGGAACACTGAGTTTTTCAGCTCCTGTAAAACTATTTTCAGGTTTATTTTCAAGT |
| | | ACATTCTTTA |
| 199 | RUNX1 | CACCCTAGAGGCAAGGACGGGGTCTGTGTCAAGAGGCTTCCCAGAGAAGTGAAAACTCTGCAGGTGCAGCCGCTGGGAGAGCATCAAGA |
| | | AGGGCAGGGTGGAGGGGCAGGGGGCGAAGGGAGGGGGTGAAGCCCGCACCCTACCCCCACATGAAACTGATTCCACTACCCCATCTCTG |
| | | CAAGCGTCCAGAGGCAGAGAGGCCAACATTTCGGGGACAGCTTGGAGGCGGGAGATTTAGGCAGGGCTCCTTAAACTTTTATGTGCATGA |
| | | AAATCAGGCCAATCACGGGGCTCTTGAGCAAATGGGGACGATGATTCAGCAGGTCTGGGCTGAGGCCTCAGATTCTGCACTTCTAACAAG |
| | | TTCCCAGGTGGTAGTGATGCTGCCAGTCCAAAGACCACACTG |
| 200 | RUNX1 | TGCTTCAGTGGGGTAAACTTGAACCGCTGAGAAGACAAGCAGGGAGTCGGTCTCGCTGAGATTTTTACCTGTGGTTCTAGGAACGCAGAG |
| | | GCATGTGAGTGTTCAGGCTTTGCATAGACCACTAAGCCACTTCTAAGAACAAGGCTACCTGAGCCATTTTGCAAAAATATGTACGTGCCG |
| | | AGGCTTTTCCTCCCCACACCTACCTCAACTCTTTCTGCCGACACACTGCACTTTTCAAGGGAACCCAAGTTTGGGTTCGGCAAGAATTGT |
| | | ACGTTGCACACCGTGTGTGATAATTCCAGGGAATTTCAATCGCATCTTGTCTTCCTTCCTAAGCAAATTCGGTGGGAACCTGGTGTGGTG |
| | | TGATAGAAAAAGCCCCGAGTTCTCTGTGGTAGACCACATCAATTTCATGTGCCAGTCTCTCAGACTCCGGCTTGCCTCTCTCAAGGAAGG |
| | | GAACAATGGTTTGCTTGGCTTCACTCCTCTCTTTCCCCCCAATTTCCACATGGGTATCTGGCTAAAAATGAGTTACAGGTTTCCTTCTGT |
| | | GAGAATTGCATGGACTGATAAAGTACCATCCCAGGAAGAAAACAAAGATGCTGTCTTCCCTTTCGGCTCACAGTTGCCGTTGGGGAGGGA |
| | | ACACACGCTGTAAATTATAGGCAGCCAGAAGTGACCGCATTGACCACTGCGAGTGGCCCAGCTATGGCAACAGGCTGAGAACTCTGGGGG |
| | | AGAGCCATTTGTTGGCAGGGATGGTGATTCTTCTAGCATCAAGCTCTAAGATGATGACCAAACGGTATCAAAAGAAATGATATTTTGCTA |
| | | CCTCTCCGGCTTGGGTGAATGATGTGGACAGTTAACCTGGACAATTTAAACCTTTATGTTGATGGATCACTTGGATGAAATTAACCAGGA |
| | | AATTGCCAAGATTTCACTTGGCCCTCTGACATCAAATCTCAATATTATATTACCAAATTAGAGATTCTAAAGAACCCTGAGTTCCTTTCA |
| | | CTGAAAGGAAGGAGTGGAAAAACCTTTCCAGATGATCCCTTTTGAGTCTTGGTGCGAGCTCAGGCCCTCCCTACACTGCCTCCGTGAAAG |
| | | CTAACCGACCCTTGTTCCTAACCTAGCGCAGGTCAGCTGAGTGTCCATCGGGCACAGGAGCCCTGGGCTTGTCCGGGAGATAGCCAGACT |
| | | CCTGCTATTTCCTGATGTCTGCATAGCTCAGCGTGTCCCTCACCATCTTTGCCGTTGGCCAGTAAGGAGAGCCCCAGGGGCCAGCACTGC |
| | | ACACTGAAACCCAACCTATTGCTCAATGGAATGCTTAAAAATTTCCTGAATCTGCCTTCCTGAGTTGATAAAATAGGAAACAATACACGT |
| | | TCTGAGGGGGTACTGAAAGCAGAGTAAAGCCAGGAAGATCTTTTTTTTCTGTTATTCTATACAAATATTGCTTCCTCTGCTTGTTAGCAG |
| | | CCCAGAGGAAATGCAGCCAGGGAGCCGTTTGCAGCTTTTCACCAGTGGCCGGTGTCTCTGTGTTACCAACCAAACGACGCTGCAAGACTA |
| | | GTGACTAACGCACGTCTGCATGATTCAACTTCACTAAAATTCCCTCTGCTGCCAGTAAAGAAGCACTTGAAAACTCTTTAATTTGAAACT |
| | | TGAGCTTGGTTAATGACTTGTTTTCTTCTCTTTCTCTTTAACTTCTCTCTTGCCATCTCCAACACACACACACACACACACACACACACA |
| | | CACACACACACACACACACTCTCTCTCTCTCTCTCTCTCTCTCTCTCTCTCTCTCATCAAGTTTTTTAATTTCAGGGACCCGGAAACATA |
| | | CAGCCCCGTGCATTCACAATAGCATTTGCTGTGATAAAGTGGCCGGCAAGCCCTCTGCATTCCCCTGCTCACTTAGCTGTATGAATAAAT |
| | | AATGAGTCACAGATACAATTTGGGTGCTCAAGAGAGTTTGTAGCCAGAAAATTAATTATTCTCCCATCCCAGCCCACTCCATCTCAGCTT |
| | | TGCCAAACCATCAAGATACACTTTGCAGGCACTGGTCAGAGTGCGTGCCCCGACGCACACGGCAATGCCTTTGAGACATTTTATGTTATT |
| | | ATTTTTGTTTGTTTAAGCACAGCCCTCTTTTACCACGAAAGATACACAAGACGCACATGCACACACATACTCACACACTCACAGCTCAAC |
| | | CACAGCTTTGTCCATTTCAAGAGGCTGGTTTCAAAAATGGAGACAGGTTTTCCACCCTGGCTGTTCCTATTCATAAGCCTGTAATCTAAC |
| | | GACTTAAGCTGCGAGAATGCTTAACTCGGGAAACTTCTCTATTGCCCTTTTCCAGAGAGACCTCGGTATGCCACAATTTGCTTCCTTTCT |
| | | CTCTTGAAAGATGCTGGTTGTCTCTTTGCATTGAGGCTACAAGGAAAAACACAGCACAGCCCCATGCTGATGATTTTAACCTAACCAAGT |
| | | CTGTCAGTCTCCTGTACTCTCTGCCTTATAGAGACAGCTGCCTTGCCACTTTGGCCCTGAAGTCCCCAGGCTGGTGCAAGGCTATCTGAG |
| | | AGCCTCCGCCTCCTGCCCCACACTGGCACCAGCCCTCCTGGCTGGCTCTGTGCATGTGCCTGCTAAGCCCCAGGGCAGGCTGCATTCTGG |
| | | GCCACACAGCATGCCGAGTTAAGGATAACTCAGACACAGGCATTCCGGGCAAGGGACAGCAAAATAAAACCCAGGGAGCTTCGTGCAAGC |
| | | TTCATAATCTCTAAGCCTTTAAACAAGACCAGCACAACTTACTCGCACTTGACAAAGTTCTCACGCACCGACTGAACACTCCAACAGCAT |
| | | AACTAAGTATTTATTAAAACATTTCTGAAGAGCTTCCATCTGATTAGTAAGTAATCCAATAGACTTGTAATCATATGCCTCAGTTTGAAT |
| | | TCCTCTCACAAACAAGACAGGGAACTGGCAGGCACCGAGGCATCTCTGCACCGAGGTGAAACAAGCTGCCATTTCATTACAGGCAAAGCT |
| | | GAGCAAAAGTAGATATTACAAGACCAGCATGTACTCACCTCTCATGAAGCACTGTGGGTACGAAGGAAATGACTCAAATATGCTGTCTGA |
| | | AGCCATCGCTTCCTCCTGAAAATGCACCCTCTTCTGAAGGCGGGGGACTCAATGATTTCTTTTACCTTCGGAGCGAAAACCAAGACAGGT |
| | | CACTGTTTCAGCCTCACCCCTCTAGCCCTACATCTCTCTTTCTTCTCCCCTCTGCTGGATACCTCTGGGACTCCCCAAGCCCTATTAAAA |
| | | AATGCACCTTTGTAAAAACAAATATTCAAATTGTTAAAGATTAAAAAAAAAAAAAAAGCCAGCGCCGCCTTGGCTGTGGGTTGGTGATGC |
| | | TCACCACGCTGCGAAACCCTGTGGTTTGCATTCAGTGTGATTCGTCCTGCCTGCTGACCACTATGCTGGGTTCAGACTTCTGACACTGCC |
| | | AGGCTACCCAACTTGTGGTTCTGTGGTTGTTTATGAGGCCCAAAGAAGTTTTCACACAACCCAAATTACAAATTTAACTGTTCCCCTTTC |
| | | CACAGCCCATCTCAATTGGTTCTTGCCAATCATGTGACTTAAGTGATGTCAATTTTTTTTTTTCTTTTCTGAGCAATGCCCTTCCTTCCC |
| | | TCCACCTGCCCTCCCCCAGGCTGTGCAAGAAAATAGCCGAGTAGACTTTGCAAGAGGGGGGGATGTAGAAAAAAGTGACTCAGTCACTTA |
| | | TTATATCTCAATGGTCTTTGCTGATTTAGTACAACTCGGCTCCTGTTGTTATTTGTGGTTTTTGGAACTACTGATTATTTTGATAAAGAT |
| | | TTCATTGCTGCTTATTCAATAGTAATTCAACGCTGGCATCAAGCCGCTGCTCCGACAGGATGTGGATCCCATCATTTAAAATGCTAGGCA |
| | | TCAGCTCCGGGAGAGTTAAGTCCTTGGTAACGTCTATCATGGCATAAGTGAAACTATAAAAGGGAAAAATAAATAAAAAGAAATGTTTTG |
| | | GTGAGAGTCTGACCCCTACAACGGGCTGGCAACTCACAGGTATTTTAAAGCCTGGGAAAGGGAAAGAATTTTACTTTTGAAATAAAAGGA |
| | | CTGTTTTAATGAAACCAAAATTATGTGGTTTTATTCCCCCTAAATGGACAACTTTAGTATGTATCTCTTTCAGTAAAGAGATAAAATCAT |
| | | AGTACAGTCTTAACACACACACACACACACACACACACACACACACACACACAAATTAGGAAGCTAAAGGAAAACAAAGCAGAGAGAATT |
| | | TCTGTATTTGGGACAAAGCAGTGGTTACTCTGCAGATGTTTATTTGTATTGTCACTTGGGAAAGCTCCCTGTATTGCCTTTCTCTAGTTC |
| | | AATTCAAATCAATAGGCTAATTTACACCTGTAGGTAAAACTACACTTTGAGCACATGAGGATGCCACAATAGAAGGGGAACCAGGAGGAG |
| | | ACACTTCTCCTGGGGCTGACTAATGAATATTATATAGCGCGTCCTCTACCTTAGAAAGACATGCCTGTTTGAAGATGCTAAAAACAGGAT |
| | | AATTTTGTAAGTGGGCAAACCACTGTGGTCACACGTATTTCATTTTCCGGCCCCACTGGCTTTACCTGCTGACAACTAAAACGTCATTTT |
| | | GTTTTGTAGTTCCAAGATGAAGAAAGGCTTATTTTCCTGATTTACTACCTTATTCATTTGGCTCTGCTCTGCCTACATCCGCCATAGCAC |
| | | TCTGCGCACGTGAAATTTCGACACATAGGGTCAAGAGAACCTGTGTGATGATGGGTTGTAAATGCCAGTCCTGGATTCTAAGCTGCAGTA |
| | | GCCAGCACAGGCACTTCAGAAAGGCTGAACTCCCACAACACTCCCTCGGTTTTCCCTCATCCACTTAATTTCACACACACAAAGACCCAC |
| | | AACGATAGTAGCTTCCATGGCACAAGTCTTTCAAAAGGAACAGACACAATTTTTACTTACTCCTGTTTTGACTAAAGCAGGAATTGAAAC |
| | | TCAACAGACCGCTTTCTCTTACACTTGTGAGAAGTTAGCTGGCCACATGT |
| 201 | chr21: | AGGGAAAAGAGATAACGAAAGAAAGAAAGAAAAAAAAAAGGGCCGGCAATTTCATGTACATTTGTTTTGGCATTCGCTGAATTCTAGAGA |
| | 35499200- | TGAAAACAATCTCCTGCTTTTAATTCAGTCCACGTGCAACAAAGTTGTACGTTGGGAGATCTGGCTTTTAATAAGAACGATTAACAAGCG |
| | 35499700 | TTTTTGATCACAGGAAGTTGAGAAGAGTCGCTGCTTCTAAGAATACAATAAACATTGACTAGCAGTTAGACGGTCCATCTTTCTCTATCA |
| | | GCCGTTTAGCAGCCTCTACTTTGATTTGGGGCAAATGCGAGATGGGACCAGGAGAGAGCTCCCCACACCCCCACCACCACGTGGGCAGTG |
| | | GTTCTGTTCCAGAGCGCCTTCCTTCCTGTCCAGGGAGGCAGGCTGCTGAGGCCGTTTCTGGGCAAGAGGCCATTGTCGGGATATTTGCTT |
| | | TAGATAGCTTGCAGCTGGGCTGAGTGGGTGTTTCATTCAGACTCAACACA |
| 202 | chr21: | AGCCTGGCGCACCCGCCCTAATTTGAGTCAGGGACCCTAGGCGCCTGCAGCTCCGGTTCGGGTTGAGTGCCTCCTGTCAGGATGTGAAGC |
| | 35822800- | TGCTGTCCCCCCCGGGGGCCTCCAGCACTGCTGAGGACTCAGCAGTCAGCCTCTCCTCCCACTTGGGCTCATTTACAGAGAGCATCTCCA |
| | 35823500 | GGAATCAGTCATGGGGAAAGGGGAAACGCGGAGTGACAACACAACACGTAGAAAGTTCTCTGCCGCCTTGGTCAGGCTTGTCAGCCTCAC |
| | | AGCCCATCCTGCTCCTGCGGGAGGAAAAGTGAGCAGAACTCAGCCCGGAGATGAGCCGCAGGCCGGCAGCCCCTGCCTCTGCCCTGCTT |
| | | GTTGTGACTGCAATGCAAGGCTCTCTGTAGGTGCGGGGGATTCGGGTTAAATGGGTCTCCAGTGGTCCAGCGCTCCCAGCAAAGGCCGAC |
| | | CACAAGAATTAGCGGGCTAGTTATTTACCATAACCATATACAAAACCACAAGCATCAGCGTTCCCTCAAATACATCCGAGACGCTGTATA |
| | | TCTCTTTATTAAAGCCTGTCAGGGTTTGTTATTGCACAGCTTGGCCTTGAACCCCAACTAAACCAGGCTGCTTGAGCAAAGAACCAAGCA |
| | | ATGCAAGCATTCAGGCAGGACCATTATAACCCTGAGGCCAAAGGCAGAAGCAGGGAGAGGAGACGTCTTCC |
| 203 | CBR1 | AGACCAGCCTCGGTCTTCGGCCTGCGGGTTCTGCAAAGTCAGGCTAGCTGGCTCTCCGCCTGCTCCGCACCCCGGCGAGGTTCCGGTGG |
| | | GGAGGGGTAGGGATGGTTCAGCCCCGCCCCGCTAGGGCGGGGCCTGCGCCTGCGCGCTCAGCGGCCGGGCGTGTAACCCACGGGTGC |
| | | GCGCCCACGACCGCCAGACTCGAGCAGTCTCTGGAACACGCTGCGGGGCTCCCGGGCCTGAGCCAGGTCTGTTCTCCACGCAGGTGTTC |
| | | CGCGCGCCCCGTTCAGCCATGTCGTCCGGCATCCATGTAGCGCTGGTGACTGGAGGCAACAAGGGCATCGGCTTGGCCATCGTGCGCGA |
| | | CCTGTGCCGGCTGTTCTCGGGGGACGTGGTGCTCACGGCGCGGGACGTGACGCGGGGCCAGGCGGCCGTACAGCAGCTGCAGGCGGA |
| | | GGGCCTGAGCCCGCGCTTCCACCAGCTGGACATCGACGATCTGCAGAGCATCCGCGCCC |
| 204 | DOPEY2 | AAACGTTTAAAATATATTTCTAAACAGAATGGGCCAATTCAGTCACAGTAACTGTTGATCTCCATAGCAGAGCAACCCACAAAGACAGAA |
| | | CTGATTTTTTTCCCATAATCAGGGGTGAAAAATATACAACTTGTTTCTGAACCAAAACCACAATTTCTGCAGTTTAAAATGTTTCACTGC |
| | | TAATATGGCCCTGGTAGAAATTATGTAGTTTCTTTTCTTCTTTAAAAAAAAAAAAAATTAAAAAAATTTCCTAAGACACTAAATGCTCCA |
| | | TCTGGAATGTAGATTCTGATCACAAAGCAGCTCAGTTAACCTAAAAAATAAAAAATTCCCATCACCTGTCTCAGTAGGGCCTGAGAGTAG |
| | | TGTGGGGAACCCCAGCTTTGGTATGGAGAGTCATGGCCCCTTGAACCAGATAGAGACCTTGAATAGCCATAGCTGGTGCTTCTCTCAGGA |
| | | TAAACTCTGATGTAGGAAGTATCACCCTCATGAGAGTGGAATTTGGTCATCCAGTTGACGCAGGGCATATTCCATGTCTTCTTTTCTGAG |
| | | ACACCCAACCATCCCCACTCCATCCTTCTGCACATCCGTGTAACAGGCATCCCCAGCTTCTCGCGTGTGATCCTTCAGGTCCTGCCAGCT |
| | | GCCTGATGGAAGAAGTCCATTTCTTCCATAAATAGCATCCTCTGCATCTCGAGGGTCCTCGAAGCGCACGGAGGCGAAGGGCACAAGGCC |
| | | GTACCGGCTCTTGAGCTCGATCTCGCGGATGCGGCTGTACTTGTAGAACAGGTCCTGCGGCTCCTTCTCGCGCACGTGGGTCGGAAGGTT |
| | | TCCCCACGTAGATGCACCCGTCGCCCTCCCAGCCGCGCTCGTGTCCGCCCAGCCGGACAACCGCACCGCCCGACGCTGCTGGCCAGCCGC |
| | | AGCCCGCATCCGCCCGTATCGCCGCCGCTGCCGCCTCAGCACGGCTGCCCCCGCAGCGTCTGTTTTGTTTTATTCTAACAGGGTCTCTCT |
| | | CTGTCGCCCAGGCTGGAGTGCAGTGGCGTGATCTTGGCTCCCTGCAACCTCTGCCTCCCGGGTTCAAGCGATTCACCTGCCTCAGCCTCC |
| | | CAAGTAGTGGGCATTATAGGTGCCAGCTAACCATGGCCGGCTAATTTTTTTTTTTTTTTTTTTTTTTTTTTGAGACAGAGTCTTGCTCTG |
| | | TCACCCAGGCTGGAGTGCAGTGGCGCGATCTCGGCTCCCTGCAACCTCCGCCTCCTGGGTTCAAGCGATTCTCCTGCCTCAGCCTCCTGA |
| | | GTAGCTGGGATTACAGCTATGTACAGCGATGTCTGCAAAGATAGGGATTTAACAGCACTCATATCTTCATGTTCATAAAAAAGTCCTACA |
| | | CGCGTGATGTACGTCTAGATCTTTCCTTTTGTCACAGGATATAGCACGGTAGTTACGGATATAGTCTCCGCAGTGCCTGGGTTTGACTCA |
| | | GCTTCCCCACGTACTGTCCTGCGCATATTTTGTGTCTCAGTTTCCTCATCTTTAAGGTAG |
| 205 | SIM2 | CACGCGCCCCGGCCTGGCTGGAGGGGCCAACCCAGCGGGGCCCGCCTGCCCGCCGGCCTTTCTGTAACTTTCTCTCTTTAAACTTCCAAT |
| | | GAATGAACGTGCCTCTTCTTACGGATTTGTTTAGATTAGGGAATAGATTCCTCGCTGATAGCGTTGCTTTGCAAATAAGACCTCCTATAT |
| | | TATTCAAACCAAACGAGTTTGTGTCTTTAAAGGACTATAGCAGCCCCATTCTATGTTAAGGGTTGGCTATTACAATTATTATATGCTTAG |
| | | GGAAAAAATGTAAGCCCCGTAGTTTGTGCTTTTCTTGATGTACAGAAAGGTTTATCTTAGGTGGATAGGTTTTGTTTTGTTTCTTAAATG |
| | | GGATTTTTTTGGTTCGTGTCTTTGAAGGGCTGTTTCGCGACGTCATTAATGAACTAATCGGTTTTCAGATTTCAAGACGGTGTGTAATTG |
| | | ATGTAACCACTGAGGAATTTCAGTGCACACCAGACTAAGACTCTTCCAGCGCAGGGGATTCCAGATGCTTCTTGGGCCCTCTGGAAGCCA |
| | | TGGGGATGTTTCCAGACCGAAAGGAGGGCTTTGCTGGGGAGCAGATGTGCTGCCTCTCCCCGACCCAGGATTTTGAGGCCATGTTTCCGT |
| | | TAATCTGGACCGAGAGCCCTCTGGGAGAGGGAGGCAGGTCGTAGGGGGCGGGGGTGAGGGGGAGCGAGATGAGGTCGTCGCTGGACGCTG |
| | | GGCTCCCTTGTCGTTGTCCTTTTCCCCAGAATCCATGGTCAGGCCTAGGGAGCCACCCCTGGGTGCTCGAGATGAGTCCCCACCCTCACT |
| | | GAAGGTCGGTCACTGGATGTTTGTGTGCATCGTAAGGGGCCCACCGAAGTCCCGAAGCCTTCTCAGGGACCAGCGAGAAAGAGGAGCAGG |
| | | CTTGGGAGACAGGGAAGGAAAATGCAGGGGAAAGGGCTCACCCCTCGACCCCAGGTAAAATTAGAAGGAACGTGTGGCAACCCAGGTGCA |
| | | GCTTTGGTCGCTCGCTCAAGGACTTTGCTAGTCACTACCATTAATTAATTAATCACTATCATTAACTACCAAGGACACCGTTTTTATTCC |
| | | CCTAAAAGCGTCACCTTGAGGGGAATGGAGAATTGGGCAGCAGCTATGCAAATCCTGGGACAGGAGACACTGCCTGAGGACCCTCTCTCA |
| | | CTCCCAATCCCAGAACCCGAAGTTATCCCCGACAACCAAGTCCAAGCACATGAACCAAGACGATCAGCTTCAGGCAGCTCCTTACCCCCA |
| | | CAAGCGGCCCAGGAGGTGGGCATTATCCCCCACCCCTGGGATTTCTCCATCCCTCCCTCTTCTCTCCTGCGGGAGAGAGAGCTGTGGTCA |
| | | CCCAGTTGGGCGCGATGGCTCTGGACTAATGGGGTCTCTAGACCCAGGGCACAAAGGCCAATCTGCCAGGGGTTACTGCATGTAATGAGA |
| | | TAATCAGACATGTTGACCAACCTAAAAGAAAAGACTCTCCCAGGGAGTAACTCCCAGTGAAATAATTTATTAAAAAAAGCAAAAAAGAGA |
| | | CATAAATTTCTCTCTACTACTTGAGGAAACAGCAAACAGAACGAATTAGGGTCTTGGCCTCTGCAGGAATAAATTATTTCCGACTTGGTC |
| | | TGGATACCTGTAATTATTTGTAAGCTGTGGGTAGTAATACTGTAATTGTCCCCCGGTCCTTTCTGGAAGTAGCAATGACCCCAAGGACAA |
| | | TTGGTGACGTCTCCACAGGGTTTACACATGGAAAGGAGTGAAAAATCGAGGAATTCTTTCAGATAGCCCAGACCAAAAATCCTCTCAGCC |
| | | ATGAAAAGGTCATATATGTGATGCTGGGCCAAGCGGACTTTTCTGGAGTAACCATATCATAACTGATTGCGGATGTAGACAAGAGCGTAT |
| | | AAACCAAATAGGCTTGAATCAACGCAGTCCTGGATTTTCTGTTGCCTCTGCTTGCTGGGGCAGTGGAAGTTCTTAAACTCCACTTCAGAG |
| | | GTTGGAAATTCTTCCCCCTCCCCCACCTCCTTAGTGACAAGGTCTCTGATCTCCTGCTGCCACTGCAATAGCCTCTCCCATCCCGCGGGG |
| | | AACGGCCGGAGTTCTTCCCTTGATCTCTCCCGAGTCGGCTTCCGCTGGGGATGGATCGCAGGTAGGCGCCGGCGCGGCCTGGGGAAGAAC |
| | | AGTTGCGGAGCATCTGAAGCGGAAAATCCAAGCAGATGTGAGGCGATCCGGGCCCGCCTCGTTCCTCTTGGGGCCTGAATTTCTTCCAGA |
| | | TAAGTTTCCTAATGGAACATTTCTAAGAGGTGGGGTACGAGGCGGCTTGCTCGCACGCGCAGTGGGACAGACTGCGGGTGGGGACGTACT |
| | | GAGAGGTCCGGACCTCAATGCGTCCGACCCGTCTCCACACCGCCCTTTTCCAGCCCCCAGTCTCCTTTCATTCCCTACTCTTCAGGCTCC |
| | | TTTGGGGCCAGTGGGTGAACCGCCATTTAGAACGGTGCCTCGGACTCGGGGGTCGTGCGCTCCATCTCTGCCTCCCCCCTGGGGCCCGCG |
| | | AGGCTGGTCCGGGCTTTCTGAGCTGGGCGTTCGGCTTTAGGCCCAATACCTGGACCAGGAATTTCTTCTCCCCGCGCCAGAAGGGAAAGA |
| | | CATAGGAGGTGTCCCAATCTGCGGTCACCGCCGATGCTCCTGACCACTCTAGTGAGCACCTGCCCGGTACTTTTCCATTCCAACAGAGCT |
| | | TCCAGCTTCATACTAACTATCCCACATACGGCCTGTGGGTATTAGCTCTAAGTGTCCTTTTCCGAGGGCCCGAGGCTCCCCCTCCAGCAG |
| | | GGAGAGCTCCGGGACGGCCCCCACCAAGGGTTGGGTTTCTTCCTTCACAATTCCACAGAGGCATCCCTGTCCTTCCTACCTGGGAAACCT |
| | | CGAGGTGCGGTGCCCGTGTACTTCTGGTACTTTGCGTGGTGCCATCAGGGACCCCAGAGCCACAGCTGCGTGTGTGTGTGGATGTGTGTG |
| | | TGTGTGTGCGCGCGCGCGCGTGTACGGCGAAAGGATGTGCTTGGGGGAGCCGAGTACACAACGTCTGCTTGGGCAGCTGCTGGGCAGGCG |
| | | TTGGGCCTGGAGGTATCTCACACCCACGTATCTTCCAGTCTTCAAACACGGCATTGCTCTGCCTCCCGTAGCGCGCTTCGAACCTGCCTC |
| | | GCGGACACGTGAACAGAGGCTGTCCCTGGGAAGATAAGTGCGCTTTCCCGTAAAATCCGGGAAATTTGCCTTGAGGAAAGTTTCCGTTCT |
| | | TGTTACTTGTCGGGTTTCTCCCACTTCCACTTAGCCATGTTTCTGCGATCTGGGTAATCCCTTTCAAGCCCAGGAGGAATTCTCCCGGGT |
| | | CCATAATTGAGGGTCGGAAGCCGTGGGGGTGAGAAACGCATTAAATCCTCCCGAAGCCCAGGAGGTGCCAGAGCGGGCTCAGGGGGCCGC |
| | | CTGCGGAAGCTGCGGCAGGGGCTGGGTCCGTAGCCTCTAACCCCTTGGAGCTCCTTCTCCCAGAGGCCCGGAGCCGGCAGCTGTCAGCGC |
| | | AGCCAGGAGCGGGATCCTGGGCGCGGAGGTGGGTCCGACTCGCCAGGCTTGGGCATTGGAGACCCGCGCCGCTAGCCCATGGCCCTCTGC |
| | | TCAAGCCGCTGCAACAGGAAAGCGCTCCTGGATCCGAAACCCCAAAGGAAAGCGCTGTTACTCTGTGCGTCCGGCTCGCGTGGCGTCGCG |
| | | GTTTCGGAGCACCAAGCCTGCGAGCCCTGGCCACGATGTGGACTCCGCAAGGGGCTAGGGACAGGCAGGGGGAGAGCCCGGGTTTGCGCA |
| | | CACCTTCCAGCCCCTGGAGGGAGCCTGCTCGGCTTCGAACGCCTTCGAACTTTTGACCTTCAAAGGAGTCCCTGGAAAAGGTCAGGAGCG |
| | | CCTGCTGCAGGCACGGTTGCCGAAGGCCAGGCCTTCCTGGCGCAGGGGAGGGCCAGGGGAGGGAAGCGGATACTCAGTCGCTGTCCGACG |
| | | GCGAGTTTTCGGAGCAGCAGGCTCATGATCCCGGGCCAGTGGCGAGAGCAGTGACACCGAGAACCCAAATCTCCGCGCCCCCATCCGCGG |
| | | CCCGGTGTCCTCCCGGCCCCTGCTGACCTCCAGGTCACGCACCCCACTGCTCCACGGCTCTGCAGCCTGTGGCACACGGCCGAGAGTCCC |
| | | CACATGATCTCGACGCCAAGGTAAGGAATTGCCCTGCGTCCTCTGAGCCTGTCTCTGGCCTGGGGGGCCGGGAAAGCTGCACTCCTGGAA |
| | | GAGGTGGGGTTATGTGACCGCCGCTGCAGGGGTGCGCGGAGGACTCCTGGGCCGCACACCCATTTCCAGGCTGCGGGAGCCGGACAGGGG |
| | | AGGGCAGAGGGGGGACAAAAGGACTCTTTAGGTCCAAAATGACCCTGAAGGAGAGTCCAGAATGCCCAGTGGCCGCGTCTGCAACGGAGT |
| | | CTTCTTTCTCCAATTGCCTTCTGCCCCATCACCATGGGCCCCACCTGCGCCACCTGCGCCCACCCTGTGACCCTGGCTCAGCGACCTTGG |
| | | CCCTTAATCGCCCAACGCCGATTCCTCAAAATTCCGGCTGCGCTGAATCGGGCTGCTTTTGCCGCCGCCCCGGCAGTTGGGCCCTGTTTC |
| | | CGCCGGCGCCCTGGGAGAGGCCTCACCACTCGGCTGGGCTCCCTGGCCCCTCCCTTCCCCTGGCCTGAGCGCCCCTGCGGCCTCCCGCTC |
| | | CTCCTGAGAAGGCGACAATCTCTTTGCACCTTAGTGTTTCGAGGACAGAAAGGGCAGAAGGGTCACTTCGGAGCCACTCGCGCCGTTTTC |
| | | ACGTGTGTGTGTAATGGGGGGAGGGGGGCTCCCGGCTTTCCCCTTTTCAGCTCTTGGACCTGCAACACCGGGAGGGCGAGGACGCGGGAC |
| | | CAGCGCACCCTCGGAAGGCTCGATCCTCCCCGGCAGGGCGCCTGGCCAACGAGTCGCGCCGCCTCCTCTCGGCCGCGCCTGCTGGTGACC |
| | | TTCCCGAGAGCCACAGGGGCGGCCTCGGCACCCCTCCTTCCCTCGCCCTCCCTGCCGCCCATCCTAGCTCCGGGGTCCGGCGACCGGCGC |
| | | TCAGGAGCGGGTCCCCGCGGCGCGCCGTGTGCACTCACCGCGACTTCCCCGAACCCGGGAGCGCGCGGGTCTCTCCCGGGAGAGTCCCTG |
| | | GAGGCAGCGACGCGGAGGCGCGCCTGTGACTCCAGGGCCGCGGCGGGGTCGGAGGCAAGATTCGCCGCCCCCGCCCCCGCCGCGGTCCCT |
| | | CCCCCCTCCCGCTCCCCCCTCCGGGACCCAGGCGGCCAGTGCTCCGCCCGAAGGCGGGTCTGCCATAAACAAACGCGGCTCGGCCGCACG |
| | | TGGACAGCGGAGGTGCTGCGCCTAGCCACACATCGCGGGCTCCGGCGCTGCGTCTCCAGGCACAGGGAGCCGCCAGGAAGGGCAGGAGAG |
| | | CGCGCCCGGGCCAGGGCCCGGCCCCAGCCGCCTGCGACTCGCTCCCCTCCGCTGGGCTCCCGCTCCATGGCTCCGCGGCCACCGCCGCCC |
| | | CTGTCGCCCTCCGGTCCGGAGGGGCCTTGCCGCAGCCGGTTCGAGCACTCGACGAAGGAGTAAGCAGCGCCTCCGCCTCCGCGCCGGC |
| | | CGCCCCCACCCCCCAGGAAGGCCGAGGCAGGAGAGGCAGGAGGGAGGAAACAGGAGCGAGCAGGAACGGGGCTCCGGTTGCTGCAGG |
| | | ACGGTCCAGCCCGGAGGAGGCTGCGCTCCGGGCAGCGGCGGGCGGCGCCGCCGGGTTGCTCGGAGCTCAGGCCCGGCGGCTGCGGG |
| | | GAGGCGTCTCGGAACCCCGGGAGGCCCCCCGCACCTGCCCGCGGCCCACTCCGCGGACTCACCTGGCTCCCGGCTCCCCCTTCCCCAT |
| | | CCCCGCCGCCGCAGCCCGAGCGGGGCTCCGCGGGCCTGGAGCACGGCCGGGTCTAATATGCCCGGAGCCGAGGCGCGATGAAGGAGA |
| | | AGTCCAAGAATGCGGCCAAGACCAGGAGGGAGAAGGAAAATGGCGAGTTTTACGAGCTTGCCAAGCTGCTCCCGCTGCCGTCGGCCATCA |
| | | CTTCGCAGCTGGACAAAGCGTCCATCATCCGCCTCACCACGAGCTACCTGAAGATGCGCGCCGTCTTCCCCGAAGGTGAGGCCTCAGGTG |
| | | GGCGGCCGGGGACGCTGGGGAGCCCGGCGGCCCCGGCCCAGGCGGGAAGCGCAAGCCAGCCCGCCCAGAGGGGTTGCCGCGGCCTG |
| | | GCGTCCAGAGCTGGGGCGTCTGAGGGAGGTTGCGTGAGGGTCTTCGGCTTCGGCGCTGGCTTGGGGCGAGGGGCCAGGGCCTTGGCGG |
| | | CCCAGGCGACCAAACCCTCTCCTGGTCCAGGGCTGGGTGAGGGCGAATTACGAATTGTTCCAGGGGCAGGCAGTCCCCCAGCCCGCACGG |
| | | CCAGCGAGTTCTTTCTGGTTTTGTTCTTTCTCCCTTTCCTCCTTCCTTCCTTCGCCAGTGCATTCTGGTTTGGTTTGGATTTTTTTCTCT |
| | | CTTTCTTTCCTTTCTTTCTTTCTTTCTCTTTCTTTTTCTTTCTTTCTTCCTCTTTCTTTCATTCTCCCCTTCCTTCCTTCCTTGGCCCCC |
| | | TCTCTCCCTCCCTCCTTCCTTCCTTCCTTTGCCAATGCATTGGTTTGTTTTCTTTCCTTTTCTGCTTTCCTTCCTTTCTTTGGAAGTTCA |
| | | CTCTGGTTTTGCTTTCTTTCTTTCCCCATCCCTTCCTTTCTTTATCCCTCCTTCCCTTCCTCCTTTTCTTTCTACGATTCCCTTTATTTT |
| | | TCCTTCATTCCTCCCTCTTTTTGTCTCTTCTGGAGGAGGTGAAGGAGGGTCAGCTTCAGGCGCTGCGAGTCAGCGGGGATCACGGTGAGG |
| | | CCCAAGCACTGCAGGCTGAGGCCACAGAGCGAACACTTGTGCTGAGCCGGGCCCTCTCGTGAGGCTGGGGTGCGGGAAGTCCGGGCAGGA |
| | | GAGACCCGCCCCCGCCGTTGCTGAGCTGAGACCCGGCTGAAAGAGAGGGGTCCGATTAATTCGAAAATGGCAGACAGAGCTGAGCGCTGC |
| | | CGTTCTTTTCAGGATTGAAAATGTGCCAGTGGGCCAGGGGCGCTGGGACCCGCGGTGCGGAAGACTCGGAACAGGAAGAAATAGTGGCGC |
| | | GCTGGGTGGGCTGCCCCGCCGCCCACGCCGGTTGCCGCTGGTGACAGTGGCTGCCCGGCCAGGCACCTCCGAGCAGCAGGTCTGAGCGTT |
| | | TTTGGCGTCCCAAGCGTTCCGGGCCGCGTCTTCCAGAGCCTCTGCTCCCAGCGGGGTCGCTGCGGCCTGGCCCGAAGGATTTGACTCTTT |
| | | GCTGGGAGGCGCGCTGCTCAGGGTTCTGGTGGGTCCTCTGGGCCCAGGAGCTGGGAGGGCTGCGCCGGCCTCTGGAGCCCCGGGAGCCAG |
| | | TGCCGAGGTAGGGAGACAACTTCCGCCGCAGGGCGCCGGACGGTCGGGGCAGAGCAGGCGACAGGTGTCCCTAGGCCGCAGGGCGCTTCC |
| | | ATAGCGCCATCCCCACCAGGCACTCTACTCGAAATCGGAAAGCTCGACCTTTTGCGTTCGCCTCTGCCAAGCCTGTTATTTGTGCTGGCC |
| | | GCTGGGTCTGGAGCTGCGCTTCTCGGCCCCTCCCCGGTGGAGCGCAGAGGGCTGGTCTGCAAGCGCGGCCTCCAGCCCCGCGGCTCCCCG |
| | | GCCCAGGAGCCAGGCGCGGGCTGACCCGGGAGCACCCGGCAGCGGAGGGGGCTGGAAGCGGACCCTAGGCCTCTCCTGTGCCACCCGGCC |
| | | CTACCGCGCGGCCGCGGGGCGCTCTCCTCTCGGGCGCAGCGGTCCTTCAGCCCAGGGCAGGTTCCTCCCTTTCCTACTCGGAACGTGGCA |
| | | AAGATACCCCAGTCCCAGCCCCTCCAGCTGAGAGCTGTTGCCCAAGGTCGTCGCTACTTGTCCGCTCAATGGTGACCCCTTGGCAGAGAA |
| | | CTAGGGATGATTCCACTCCGGTTGATGTTTTAGGGGAAATTAAAAGAACATTCGGTTTTCTGAGTCTCCTTCCGGGGAGGCGTGGTGGTA |
| | | ACTGGTTTGCTGGGAAGAGCCGTTCCTTAACCGCATGCAACAAAGCAGGTGTGGAATCCGGACGAGAGGGCACTCACTGCCTTCTGCCCC |
| | | CTTTGGAAATAGAAAAAGCCTTCGAAGCAGCAATCCAAAGATCAAATGATTTGCGGTCAATGATTTCAATTAAACCAGAAATTAGTAAGG |
| | | GAGGGCCGAGAAGACACGGCTGCTCAGAAGCTGTTCGCTGTTTGAGGGATTTCCCGGAGAGCCTGTTAAAAGATGCGAAGTGGTGGGTGT |
| | | ACCGCTCAGCCACCTTTAAACCGGCTCTGTGCGTTCTGGCTCTGGAAAGCAAGTCTCCAGGCATTTGGGCTCAGAATTGCTGGGCCCCGA |
| | | GTTTGGGCGGGGGTGGTCCTTCTGGGGGTCAGGCCTTGAGCAGCTTGCACTGGTGGCAGGTTTGGGAGCAGTTGAGGGGCTTCCTGTGTG |
| | | TCTTTTGGAGGGGGTGACCCTGGAAGTTGGCACTCTGGAAGGGAGCTGTTTGGCCCTAGAGTTTTGGAAAGGGCCCTGAACCTGTTCGGT |
| | | CCCCCTCGGAAAGGGAAGGGAGCAGTGGCTTAGTCCCTCCCTCCTCCATTCGTGCAATGCCTGGGGTAGGGGTAGACCTGGAGCCGGTGG |
| | | ACTCATATCCTTGGAATTCGTCAGGACAGCTGCTCCGGGGCCTTGGCCCTCAGTCAGTCTGGGGCTGAGGAGTAGGGAAGCTGGGAACTT |
| | | GGGGCAGAGGAAGAAGATGCGTTTAGAAAGACCTCCATTATGCAAACTGGAGTCCATTTATGCAAACTGGTCACCCTTCCAGTAGCTCCA |
| | | AAGAGTGGCAGTGGAGTGGCATCTTGATTGATTTAACCTCTTCTCAGGGGACCTGGGTCTGCGAGGGAGGATATGGCTGCGGGGTTGGAA |
| | | TAGGATCTGTCTGAGCTGCCAGGGTCAGGGTGGTGGCCCTAGGGAGGTTTTAGGGCCAGGGTGGTCCCGGGCTGTGGCAGGGGCTCTCAG |
| | | ATCGCCTCGGGCTCTCAGCTGCAAGGTGAAAAATACCATGAGGAATTGATCTGCCAAGGGCGGTCTTGTCTCAAAGCAAGTGGATTGCTG |
| | | GGGTAAAGAATCTAGAGACCAGCTTAGGACTCTGGGAGGAAGAAAAAAAAAAAAAGAATAGCATAGTCCTAAGGAACTGCAAGGATCACC |
| | | AGATTAACCCTTCATACCTGGGGAAATTAAGGCCAGACATGACACAGGCCTTTCCCAAGGCTCTGTAGCAAGGGCAATAGCAGGCCAGTT |
| | | GCTGCCACTGCGGTCCTGTGGGGCATGTTCTCACTCCACTGCACCCAGGAGGCTGCCAGCCTCTGTTCCTTTTAACATAGATCTCCTCAG |
| | | TTGTTAAGACAGAAAGAGGAACTCAGAGGGGTCCCTGTGTGCAAGGCAGAGGGAGACCACCAGAACCAGGGTAAGCACCCCACTTGGTAG |
| | | CCAGTTCAAGGACTTGGGGATGTTTTCAACATTTACAGCGAGGTTTGAGGCCCCATTGTCATGCAGCGCTACTCGGCCTTGGTCTCCTTA |
| | | TCTGTAAAATGGGCCCATTAGCAATGCACAGGGTTGCTGTGATGAAGGGTGAGGTCCCACAAGCAAAAGCTGTGCAGTGAGGGGGGAATC |
| | | CTAAGCATTGTTCCTATGCCATTCACCCCTTCCTGTGAGCTCCCCATATTCCCTGGCTCAAAGGAGTCTTGAATGGCAGGGATGGAGGAC |
| | | TCACTGCCTGGACTTTGAAGACCCCTGCTTTCTGGGTGACCACCTTTTCTTCCCTTTGACAGTGAACTAATACATTGGAGGTAGATAGTG |
| | | CTGGGAAGAGGACAGGAGACCACGGCTGACTTTGGACATGGGCTCGAAATTGATAACTTGATGAGTCTTGGAGGGTGGTTAAGATAAGCT |
| | | CGGGGCTGGGGCAGCGCTGAGGTCTGATGGTCAGCCAGCCCTCCCCAAAGTGTGGCCCTCCGTTCTGGAGATAGGGGCTTTGGAAACTGC |
| | | AAAAGCGTCCTGGCAGGCCAGCTCTGGTTGCTCCCTGGCCATAGCTGCTCTGACTACAGGCAGCAGGACGCAGGTCGGCCTCTGCCCATC |
| | | GGAGGTCAGAGGCAGGGCCTCCAGCACCAGACTCAGCAGTGCCACTGCAAACCTGGCACAACAGGCTGGTCCCAGGACTCAGCTCAGCAG |
| | | TGAAGTTGGAAACCAAGGTTGAGTCTCCCCATCTCCCTTTCCCCAACCCGAAAGACCCAAGATGGGTGTGGGTGAAAGAGGGAGAAAGAA |
| | | TTGCTACTCCAGAAACTGTCATTTGCCCACACGAAACGAGGTGGGGTTCAAGGTCTGAACTCTTCCAGTGCCTGGGTGCCTTTGGGTTTA |
| | | AATTCAGCTGCAGGTGCCCCCATCACCACTTCCACCTGAGCACACCACGAGAAGCCAGGTTATCTTAGAAACTGTTTCCCGGAATCAAAG |
| | | CGACTTGATTTGGAGAGTTGGGTGAGGAGAAACTCACCCCTATACCCCTCAGGGCGTCAGAGATGTGAGGCAATTCTCTACCTCCGCTGG |
| | | AAAAAATGCAGATTTATTAAAGGTCGACTGTTTAGCAGAACAACGTAGATTTTTTACAACGCTTTCCCCGTCTCTGCTTTGAAGCCTGCC |
| | | AGGCTGCAGCTGGGGATCCAGGAGGGAAAGCCCGCAGGCGCAGAGGGGACAATCCGGGAAGTGGTAAAGGGGACACCCGGGCACAGGGCC |
| | | TGTGCTTTCGTTGCAGGCGAGGAAGTGGAGCGCGCGCTGCAGATTCAGCGCGGGGCTAGAGGAGGGGACCTGGATCCCTGAACCCCGGGG |
| | | CGGAAAGGGAGCCTCCGGGCGGCTGTGGGTGCCGCGCTCCTCGGAGCCAGCAGCTGCTGGGGCGGCGTCCGAACTCCCCAGGTCTGCGCA |
| | | CGGCAATGGGGGCACCGGGCCTTCTGTCTGTCCTCAGAATACGTAGGATACCCGCGGGCGACAAGCCGGGCCAGGCTAGGAGCCTCCTTC |
| | | CCTGCCCCTCCCCATCGGCCGCGGGAGGCTTTCTTGGGGCGTCCCCACGACCACCCCCTTCTCACCCGGTCCCCAGTTTGGAAAAAGGCG |
| | | CAAGAAGCGGGCTTTTCAGGGACCCCGGGGAGAACACGAGGGCTCCGACGCGGGAGAAGGATTGAAGCGTGCAGAGGCGCCCCAAATTGC |
| | | GACAATTTACTGGGATCCTTTTGTGGGGAAAGGAGGCTTAGAGGCTCAAGCTATAGGCTGTCCTAGAGCAACTAGGCGAGAACCTGGCCC |
| | | CAAACTCCCTCCTTACGCCCTGGCACAGGTTCCCGGCGACTGGTGTTCCCAAGGGAGCCCCCTGAGCCTACCGCCCTTGCAGGGGGTCGT |
| | | GCTGCGGCTTCTGGGTCATAAACGCCGAGGTCGGGGGTGGCGGAGCTGTAGAGGCTGCCCGCGCAGAAAGCTCCAGGATCCCAATATGTG |
| | | CTTGCGTGGAGCAGGGAGCGGAAGAGGCAGCCGGTCCTCACCCTCCTCTCCCGCCACGCACATATCCTTCTTGACTTCGAAGTGGTTTGC |
| | | AATCCGAAAGTGAGACCTTGAGTCCTCAGATGGCCGGCAACGCGCCGAGGTCACGCTCCCCAGAAACACCCCTCTCCCCTCCCCTACCCC |
| | | AGCTCCCCCTGGGGCGGGTGGTAATTGGGGGAGGAGAGGCCGCAGGCAGGGAAGGGGTGGGAAAGCCAGAGAGGGAGGCACAAAGTGATG |
| | | GCAGCCCGGCAAACACTGGGGCTTCGGGCTGGGCCGCGCTCGTTTAATCCCACAAAAATCCCATTTTGGAGGTGAGAAATAGAGGTTAGA |
| | | GGTCGGGCCCTTCTGGAGATCAGACCGAGGAGACGGGCCCAGCTGGCGTCTTAAAGCAAGGAGGGGGAGTCGGGAGGAGGTGAGACCCCT |
| | | GCACCCAGGTGGGGCTCCCAAACCGTTCTGGATTTACCACACTCCCAGGTCCGATTTTCCATGGAGGGCTGGGGTTAGGGACTGGCACCT |
| | | TCTTGTTGTTAACCGCATTTGATATTCACAAGAACCCTGTGAGGAGACTTTGTCACCGTTTTTAGATGCCTGAGGTTGCCGGAGGGGCAG |
| | | TGAGAGAATCGTCTAACCTGGTGTTCCTACCACAGTCCAGGCCCTGTGTCCTGGGCTGGACCCACAGCCCCTGCCACCACCCAGAGGAAG |
| | | GCGCGAAGCTGGCTGCCTCCTTTACGGGTCTCCCTTAGGTGCCCTCATGAAGGGGGACGGCCACCTCACAGTGCAGGAACTATCTCCCCG |
| | | TTTGCTCCCAAATAGTCTTCTTGGTGTGGTGCTGTCTATGGTCTGTGACCTGCATCTGGAGTTACCCCCAGGACCAGCTTCGGAAGAGGA |
| | | GGGATCGCTTGGAGGCCGTGCAGTGTGAGGAACGGCAGGCAGGGTGTGGGACCAACATGCACACACTCGCAGGTGCTGGGGCCAGGGAGG |
| | | AATGAGGCGCTGGCTCCCTTTCCCTCCATTTCTCCCTGGGGGTCCCAGCAACCTGGCCATCCCTGACTTCCAACAGCACAGCGTCCCCAC |
| | | AGGTCCTGCAGTGCTCTGCAGGGGTGCAGGGAGCTCCCCTCCCCCCAGCCGCAACCTCACCTTCCTCACCCCCACCCCTCCGGCAGGAAA |
| | | CCACAGGCTGGGTTGGGGACCCCTGGTGCTCCAAGAGAGCAGTGAGTGCTGGGAGCCGCTAACCCCGAGGCGCCTAGCACAGACTCTTCT |
| | | CACCCCTTATTTCTGAAATAAAGCCCTTCCTTAGGTCCAGATGAGGACCACGTGCTCAGTGCCTCACTTTCGTGGGAGTGTATATCACTT |
| | | TACAGTATCAAGACAATTTTCTTTCGTTACAAATCTTTATTTAGTCTCTGCGTTTAGACCAAAGTAGATTTTTATGGGCTGAGTGAAAAA |
| | | ACCTCGCCCGCATTGGTTTCTGATGGAACAGCTGGCAGCGCCACGGCCCCGGGTGGGGTGGCCTAGAGGCAGGGGTGCTTGGGAGGAACA |
| | | TCTAGCACCCGACCACCTCCACCAGGTGGGAAAGGGACGTTTGCACCAAATCTCCGCCGGCAAAGCAGAGGCTTTGGGGAATTACAGAAA |
| | | AACTATAATGATCTAAAAGAGAACAAGTTATCTTGAACTGTGCGGGTATTTGAATCATACAGAAAATTGTCCTGTGTGCCCAATGCACTT |
| | | TTGCATGTAGAGCCAGGGCCTTCGAGGAAGCTTTCAGGAGATCCCGGGCAGCGGAGTCTGGTCTGGAGTTTCATTTCCGTAGGTGCAGAT |
| | | TTCTCCCCAAGTCTTCCCGCCATGGGCTTTGCAAGAAGCCAGGGCCCAGAGGCCACGCTCACCGTTAACACTGCACAGGGCAAAGGTGGC |
| | | TCCAGGACAACTGCCCAACCCCAGGAACGACCCAGCAGCAGAGAAAAGGACAGCTGCCAGGGTGCCTTTGTCGCTTTTTGGAAATCAGAA |
| | | TTCCTGGGTCCTTAGTTAAGTCTTACTTCACCAAATCCCAGGACCTTCACATTTTGGTTCTTGCCATTGCTAACAGTTGTAAATGCTGCC |
| | | GCCACGAGGCCTGGGAGGAAGGACCCGCTGGTGAGAGCACAGGGAGTGCTGCTGTGATCACGGTGGTGATGCGGGGTGAGCGCGATTTCC |
| | | CGGGATTAAAAAGCCACCGCTGCCCCCGTGGTGGAGGCTGGGGGCCCCCGAATAATGAGCTGTGATTGTATTCCCGGGATCGTGTATGTG |
| | | GAAATTAGCCACCTCCTCAGCCAGGATAAGCCCCTAATTCCTTGAGCCCAGGAGGAGAAATTAAAGGTCATCCCTTTTTAAATTGAGGAA |
| | | TAGTGGTTTTTTTTAACTTTTTTTTTTTTAGGTTTTTAGTTGCCGAATAGGGAAGGGTTTGCGAAGCCGCTGCCCTGGGCCGAGGTGCAT |
| | | TTTACGCTTCCAGAGGTCGAGGCCTCCAGAGACCGCGATGCCCAGGGCGTTCCCGGGGAGGCTGAGAGACCCAGGGTGCTCTGGGTGACT |
| | | GCACGGCGACTCCTCGGGAACCCACTCGTGGCTGCCCGCTTGGAAGGGCTTTGCGGCCCCGGGAACGATCTCCAGGATCTCCACGGCTGG |
| | | TCAGGTTCCCCGTCCCTCGTATCCCGCGCTGCCCGGGGGCTCCTGCCTTTGGTTCAGTGCTCGCGGCACCACCGCACTCAGGACGGCAGT |
| | | GGGGGGCTGGGGCTGGGGCTGGGCCTGGCCCAGCGTGGGTTGGGGCGGGGGACGCGCCAGCAGCGCCCGCAGCTCGCTCCGCAGGGGTCG |
| | | CAGCCAGGGGTCGGGAGCTAGGCTCGTGGGCCGGGAGACGCCGGGCGCGTTGTCCTCCGGGGAGGTTGGGGTGCAGGCGGTGCACCGACC |
| | | CTCGCCATCTGGCGCTGCAGCCACCAGCCACGGCGCTTAGTGGAGGGTCTGCGGCCAGGCTCCCGGCGGAAAGATTCCGGGGAGGGCTCG |
| | | GGGGTTGTCCCAGCCCGCGCTAAGCGCCGCAGCCTCGCCCGGCTTTCCTGCTTCCTCGGACTGTGCAGGGGAAGCCTGGGGTCTCGCGGG |
| | | GCGCAGCAGTCAGGTCGAGGGTGCAGCAGGAGGGGAGTCCTGACGGGCAGGTCCCTCTTTCCCCTGGTGCGCAACACTGGTTGGTAGCTT |
| | | TTGCGGAGGTGGTGAAGAAGGGCAGGAGGCCTGTTGAGCGGAGGAGTCCGGGGATCCCTAATTATGTGACAGGAGACCCTTTCCAGTTCG |
| | | GCCTGTGGCCCATCCCTCTCTCACCGCCGGCAGATTGGAGTCTGCTCTCGGGGAGCCCCCAGGTAAACCCCTCACAGGGAGAAGGTTTCG |
| | | GATTGGAAGGAGGACCGCGCTCGTGGGGCGCCTGTGAGAGCTGGGAAGCCCAAGGGGTAGCGTGTAGGGGGTTTTTTATGCGGGAGGAGC |
| | | TGCCTCCTGGGCGGCGGGGACTTTCTGTCTCAGCCTGTCTGCCTTTGGGAAAACAAGGAGTTGCCGGAGAAGCAGGGAAAGAAAGGAGGG |
| | | AGGGAAGGAGGGTCCTTGGGGGAATATTTGCGGGTCAAATCGATATCCCCGTTTGGCCACGAGAATGGCGATTTCAAAGCAGATTAGATT |
| | | ACTTTGTGGCATTTCAAATAAAACGGCAATTTCAGGGCCATGAGCACGTGGGCGACCCGCGGGAGCTGTGGGCCTGGCAGGCTCGCACAG |
| | | GCGCCCGGGCTGCCGGCCGCTGCGGGGATTTCTCCCCCAGCCTTTTCTTTTTAACAGAGGGCAAAGGGGCGACGGCGAGAGCACAGATGG |
| | | CGGCTGCGGAGCCGGGGAGGCGGCGGGGAGACGCGCGGGACTCGTGGGGAGGGCTGGCAGGGTGCAGGGGTTCCGCGTGACCTGCCCG |
| | | GCTCCCAGGCATCGGGCTGGGCGCTGCAGTTTACCGATTTGCTTTCGTCCCTCGTCCAGGTTTAGGAGACGCGTGGGGACAGCCGAGCC |
| | | GCGCCGGGCCCCTGGACGGCGTCGCCAAGGAGCTGGGATCGCACTTGCTGCAGGTAGAGCGGCCTCGCCGGGGGAGGAGCGCAGCCG |
| | | CCGCAGGCTCCCTTCCCACCCCGCCACCCCAGCCTCCAGGCGTCCCTTCCCCAGGAGCGCCAGGCAGATCCAGAGGCTGCCGGGGGCT |
| | | GGGGATGGGGTGGTCCCCACTGCGGAGGGATGGACGCTTAGCATGTCGGATGCGGCCTGCGGCCAACCCTACCCTAACCCTACGTCTGC |
| | | CCCCACACCCCGCCGAAGGCCCCAGGACTCCCCAGGCCACCTGAGACCTACGCCAGGGGCGCCTCCCGAGCGTGGTCAAGTGCTTTCCA |
| | | ATCTCACTTCCCTCAGCAGGTTCCACCCAGCGCTTGCTCTGTGCCAGGCGCCAGGGCTGGAGCAGCAGAAATGATTGGGCTGCTCTGAGC |
| | | TCTGAAGCATTCGGCCGCTGTGTGTGTGCAAGGGGCGCAAGGACGGAGAGACAGCATCAATAATACAATATTAACAGGAGCACTTGTCCA |
| | | GAGCTTACTGCAAGCCACATTCAGTTCCGGACCTTATTGACTTCCCCCTCCCATCTAGAGTGGATTCTGGTTTTTCAATTTGTTTTGTTT |
| | | TGTTTTTTGTTTGTTTGTTTGTTTTTGAGACGGAGTCTCACTCTGTGGCCCAGGCTAGAGTGCAATGGCGCGATCTCGGCTCACTCCAAC |
| | | CTCCGCCTCCCGGGTTCAAGCGATTCTCCCGCTTCAGCCTCCCGAGTAGCCAGGATTGCAGGCACCCGCCATCATGCCTGGCTAATTTTT |
| | | GTAGAGACAGGGTTTCACCCAGGCTGGTCTCGATCTCCTGACCTCCGATGATCCGCCCACCTCAGCCTTCCAAAGTGTTGGGATTACAGG |
| | | CGTGAGCCAACGCGTCCTGCCTTGATTCTGTTTTTAACTCCATTTTTTAGAGGAGGAAATTGAGGCACAGAGAGGTTAAATAACATGTCT |
| | | AAGGTCACACAGCAAGGGGTGGAGCGGAGTTAGCCCACTGGCCTAGCTCTAGAGCCCACCCGGATAACCAGAACTTGGTGAGGCCTCCGG |
| | | GCTCTTGCTTGGTTTGGAGCCAGGTGCTTAGCGCCCCGAGCCCGGGGCCATTCACCCTGCAGGAGCTGCACGCGCCCCTGACCTCGGCTT |
| | | TTCCCTGGCAGCAGAGGGGCTTTGCGGGTCGGCCGGGTAGCCCTGAGCACAGCTCGCCACTTCCAGGTGGGCTGTTGGCGCTGGCTGGGG |
| | | ACACATCCCGATCTTTCAAATGCCCTTTACAGAGCCTCATCAACGACCCGATTCATTCCCCCCTCCTGTCATTTGTCTCTGCCATCGAAA |
| | | AATGCCTACCGAGAGCTGCTCTGCATTTCCGCCCTCTATTTTGTGTTTTACTTTAAAATAATAATAAAAAAAATGTTGGCTGCAGGACGC |
| | | CATGACTTAGGTCAGCGAGTCAGCCGCTAGCTCTGCATTTCCAAAAAGCAGATCTTTTCACAACTCTCTTGCCCCAAGTGCCCTGGTGTG |
| | | GTTTATTTTTTAAAATGCATGCCTGCGGAAGAGAAGACCCGGGGAATATTCGAAACCCCGAGCTTTTACAACATAAAGCGCATGGTGTGG |
| | | CCGCGGCGAGTAATGGCGCT |
| 206 | HLCS | CAAATCACTTGAACTCAAGTTCAAGACCAGCCTGGGCAACATGGTGAAACCACATCTCTACAAAAGTAAAGAAAATTAGCCAGGCATGGT |
| | | GCTGTGTGCCTGTAGTTCCAGCTACTCCTGGGGAGGTCGAGGCTGCAGTGAGCCGCAATCACGCCACTTGTACTCCAGCCTGGGCGACAG |
| | | AGCAAGTCCCCATCTCAAAAAAAAAAAAAAAAAAAAAAAAAAAAGGCTGGGTGTGGTGGTCCCAGATACTCAGAGGCTGAAAAGGGAGGA |
| | | TTGCTTGAGCCCAGGAGTTCAAGGCTGCAGTGAGCTGCGATCACATCAATGCACTCCATCCAGCCTGAGCAATGGAGTGAGACCCTGACT |
| | | ATATTTAAAAAAAAAAAAAATAGGAAGAAACAACTCAACCACAGGGCTAGTATGTTACTCGGTTATAAAATGATAAAGCCCTAAACAGAG |
| | | AATTAGCCCGTTTCCAGAAGAGGCCAAGAACAGATGATACAGCTGAACTGAACTCCTGCCTGTACAGCTCGTTTTCTACAAGATTCCAGA |
| | | CCTGGAAGATGATGGCATCCAGCCCCCATTGAAGCACCTCGAACAAGAAAAACGCCGAGTCCGAAGAGCCAGGCCTTGAACACACGATTC |
| | | CTGTCTATAAATAACTCCCCCTGGGGAATAAAAAGCAGGATCCAAGGCAGGAAACCCGAGCCGTGGAATCTGGTAAGTTCTTAGGAAACC |
| | | CACTCACGGGCCTGAGTCCCCCGTGGAAGCGGCGACTTCGGCACCTGGACACCCGAGTCCCCAGAGCCCCGGGCGGCCGCGCGTCCCTAC |
| | | CTGCAGGCCTGATACCGGCCGCGGAGCGCTCCTGGCCCCGCTCCCGCCAGGCTCCGGGACCGCTGAAACGCACCCAGGGGGGTGAAGGCG |
| | | TAGTCGCCAAGGACAGCGCAGATGGCAGCGGAGGCATGGGAGCCGGAACCTACCGTGGCAAAGGGCCAGGTCGGGACGCCCCTCGGCGCA |
| | | GCCCCAAATCCTGCCCGCGCCCCAGCCCCGCTCAGGCCGCGCCCCTGCCACCTCTGGCCACACGGGCTGAGACGTCTGGCTCCTGCACAG |
| | | CGCACTTCCCGCTGCCCTTCTCCACTGGCTGCTCAGGCCCTGCCTCGCCAGCACGGCATCCGCGGGGGATCCCTACCTGTCCTTTAGGGC |
| | | TTGCCTCATAGGTCAAACGTCACCTCCCAGGGAGGTATGGCCTGCCCCCTGGCCAGGTGGGCCCCTTCCACGCTCGCCTGCAACACCACC |
| | | CACCCACCTTGATAACTGCTTGTAAAGGTTGTACTGCTTTCCCCCTTGAGACTGCAAACCTTCAAGGGCAGGAAATGGGTCTGTTTTCCT |
| | | GGCAAAATAATGAAGTTGGCTTAAGGTTTTGCTGAATAAAATGAGTGACAGACAAAAGTAGCCAAATTTGGCACTCCTGATGGGTTATTT |
| | | GATGAAGGAGGTGCAATGTATGGGCTTAACTAGTTATTCTGGATTTCTTTCCCCATGTTA |
| 207 | DSCR6 | CAAGGCCGGTGCACGCGGACCCGAGGATTCGGTAGATGTCCCCGAAGACCCGCTGCCGCTCTAAGGCGGTGGAAGCGAGATTCTCCGGA |
| | | AACCCAGGGAATCCGATGCTCGCACAGGACCAAAGCCCGAGGCCGCGGGGACCACAGAGGGACGGAGAAGCCGGGACTCCTCACATCC |
| | | CACATCCGGCAGGGGAAGCCCAG |
| 208 | DSCR3 | CTGATAATAAAGTTTTACCATTTTATAATTTAAAAATGTAAATATGGAGTTGGGCATGGTGGTTGGGAGGCTGAGACCAGAAGATCGCTT |
| | | GAGCCCAGGGGTTTGAGACCAGCCTGGGCAACATGCAGAAACCCTGTCTCTACAAATAAAAAATTAGCCAAGCGTGGTAGCACGCACCTG |
| | | TAATCCCAGCTACTCGGGAGGCTGAGGCAGGAGAATCGCTTGAGCCTGGGAGGTGGAGGCTGCAGTGAGCTGAGACTGTACCACTGCACT |
| | | CCAGCCTGGGTGACAGAGTGAGGCTCTGTCTCAAAAAAACAAAACACAAAAAAACAAACAAAAAAAAGCAAATATATGTAAAAATAGGAA |
| | | GTGCGGTTTCCCAAAATGAGGTCTGTAAACAACTGATCTAGAAAATGTTCTGGAAAAAGTAAAAAAGGATCAGGATCTGAGGTCAACTGA |
| | | CCTCTCCCTGCGCTCTGGACAGGCAAACAGGCAAGGTTCCCTCTGAGGCCGTAGCGGCTTCTCGTGGGCGAGTCCCTGTTCGCAGGTGAC |
| | | GTGTGGACCACGCTCTTCCGAAGCGTCTGGCCTGTGTGCTCTCGGGGAGGGGACGCAGGTCAGCCCACCTAGCCGATGGCTAACAAGTCA |
| | | GTTTGTTTTCTGAACGGAAGCTTAAACCTAGAAAAGTAACTGGGTTGGGGTGGGGGTGTAGCCACATGCAGTAAAAGCACTGCCTGTCTG |
| | | TATAACAACGACCTGATGAAAAAAGGAACGCGTGAAATGGGGAGTGTTAGGGCGTCACAAACTCCAGTGTGGTTGAAATGAAAGCAGAAA |
| | | GCAAATGGCAAGCTGGCTTCCCCTTCCAGCTTTTCACAACCCTGCCTTGCTCATGGTCAGCCCCAAGCACGGGCGGAAGAAAGGACTGGA |
| | | GGGGAGGGAAAGGGGTGGGGAGCGAGGGTACCAGAGGCGTGGGAGGACGGGGACAAAGGGGCAGCAAGGGACCGGCGGAAAGGAAAGTCG |
| | | GCGTTAGCTGGATTGGAAACAGTCCAGACAGAACGATGGGCTCTGCTGCCTCCGGGTGGGGCACCAAGCGGGGAGCGGGGCCACGAGGCA |
| | | GGGGACAGTGAAGCACCATGCAGCGCCCACCAGCCGGCAGCGCCCACCAGCCTGCGCTGCGCTGCACATGGTACCCGCGGCCCCAGCTGG |
| | | CCAGTGTGTGGCGGAGATGAGACCCTCGTGAAGAGACTAAGCGGCCACAGCAGGGGGAAGGGTTGCTCACATAACCCCATACTGCTCACA |
| | | CTACGAGGTTAACTGCCGTGAGATCTGCCTGCAGCCAGCAGAAACCCGTTCTAGGAAAACGTTGCCCAGTGACTTCAGTGAGTGCCACTG |
| | | ACCCGGGCGCCTCCGCCCCGGCGTCCGGCAGCAGCACCGATTGCGCAGGAGGCACCTTGCAAACAACCTTTCCTGATCCGCGCTGCAGTT |
| | | CCCAGGCCGGTTGCAGCCGTTTCACAGAGACTGCGCACACAAAGCGTCTCCGTGCCCTGCCATTCACCTTTCGACACAGCCGCAACCCCT |
| | | CTTTTCAGTGTTAAAACCTGGCGCCAAAAGGAACATGCGATGTGACGTGTTACCTCTGCGCATGCGCCGGGCATTCCCAGCGCCCCGAAC |
| | | CTGATGAACGCGCGGTGGGGACCCCAGGCTTCCGTGCTTTCGTTTTCCTGGAAGCTACGTGTCCTCAGTCTACATATTGTTACCTGGAAA |
| | | ATAAAGTTTTCTCCTTTTTTCTTCCTTTGTTAACAGGCAGAAGGTGTAGGCTGCAGGTTTCGGGCCTAAGAGAGGGCATGGCTGGCGACA |
| | | CGGAGTAGACTCCTAGATGACATAACGGAGGCGAGTCTGCACCGGGGACTCGGCATTAGGAGGAGGCAGAGGAAAAGCCCACCACCGTGG |
| | | CCGAGGGAGATCTAGCAAGCAGCTTGCAGGGGGTGAAGTGTGTGCAAAGCAGGCTGAGACCTGTCCAGTATCGAAACACGCCGCGGTGGT |
| | | CAAGCAGGCTTTACCATGCT |
| 209 | chr21: | TGAGGCTCAAAACAGGTGTCTGTGAGCTTCACAGGCGGTAAGGCCGTGTCTACATGGCCGGGACATGCATCCCGGGGCTGCCCCTGCCG |
| | 37841100- | TGCTGCCCGAGTGCACGGGGGATGAGGACCTGACAAGGCCATTGATCTTGCGGGAGCTTCCTGAACTACTCCAGCGTGAAAATCTTCCAG |
| | 37841800 | AAGGATTCTCCACAGGGCAATGAGGCAAGAAATTTACAGCTTAGCCTGATTAATGGGCCAGGCAGTTAAGAGTTCTTTGCCAAGCTATGA |
| | | GCATAATTTATAGTCATCACGGCAGGAGGAAAGGCCACATAACTCACATCCTTAAAGGGCCCTTAGAACAAGAGACACGCCGGATCATTG |
| | | AAAACGTCTCCACTCCTGGCGCCAAAAGAGATCGGCACGTTTCTGGGTATTCTGGTCAAAGAACAGGGAGTCTGGATTAATATACACGGC |
| | | AGAAAAAAGCGAAGAAAAGACACACAGGTCATATATTTCTGACTGATATTCCGTTTGTTGTTTTCGGAGGGACTTGGTATTTATTTAACC |
| | | ACATTCTCACTTGACACGCCCCCTCCCCACACCTTGTAAATGCCTTCCTCTTTAGCCGAGTCATTTTTCATCACATAGAATTGAAATGTT |
| | | GCCAGGAAGGCGGTTTATGAGATTGTAGAAATGGCACTAGAGAAAGCAGTGTGAAAAGAGGCCTAGAACGT |
| 210 | ERG | TCTCTACATGCTATCTACTAAAAACTTAGGCAAGGAAATGCATCAGACCAAACACCCCACAGCACAGAGAACCGACCGGCCATTGCTTTC |
| | | CAATCTCCGCAAACCTAACCATTGCTGGAAGAAATCTTACTCACAGTGCACAGACAGTAGGTATTTTATTGAAGATAAACATATAGTGGA |
| | | ACAAACCAAATTACCCCCATTTGAGTTACGTGAGCACTCAGTTCTCAGCGTGGATGTCCCACAAATCAAGTCAACATTTGCGTCCCATTA |
| | | CCAGCAGCCACTTGCCGAGTATCTCTTCGCTTCCACTGGGACTGCCTGGCATCCCTGATGCTAAGGAGCCACTGAAGAGCCTCCAAATGT |
| | | CTGACATTCACAAACGCATCTTTTGCTTTGACCCGACCCTTCAACCTCTCCGAGTCTGCTGCCTTTTCTCAGACACACATCCAGGCACCG |
| | | TTAGGGATAGTTAGAGAATCTGAAAATTCAGAAGCGCTCCGAAAAGCCTTTCCAAAAGTAATCCACAGCACTCAACAGTGAATTTAGAAA |
| | | CCCCAATTTTTTTCTGAGTTTGAAGTTTTTAAGCCTTGCGGATGGTTGGAGTAGGAAAAA |
| 211 | chr21: | TCAGACAAGCTCTGTGCAGTCGGAATTTTTTAAAGATGCACTGTCACTTGAGGAAGACAGGTGATCTTCCTGCGGCACAAATAGAAGCAA |
| | 39278700- | AGAGATTTCTCTTCTTCTCTGTAGAGCAACACAATTGATAAATGGCCGATAATCTCCACCAAATTGGCAGCAGTAGGCTGCCCGAAGGCA |
| | 39279800 | GCAGGCATATTCGTCTTTGTGAATTGTTTTACTATGATGCTGTCACATTTCCAGGAATAAGACGGTTAAAATGATATATTGTTGTGGTTT |
| | | GGCATTTGCAGCTTTGCTCTGACTTCCCTGGTAACTGCCAACATCTGCAAATTATTATGTGCTTAAAAAAAAAATCAACCGCCACCGCAG |
| | | GCTGCCCCCACGGTCCCTGGCTGGGCCAGGCCTCCTGCCAGGCCACAGGGCAGAGTTCTTGGACCAGGAGGCAGCAGGGTCAAAACCCAG |
| | | GTTGCCTAGGAAGCCCCCAAAGACAGTTATGGATAGAGCTGGGAGCCCGAAACACATGCGGCAGTCTCTCAGTTTCCAGGTACCGGTTCT |
| | | CACATCATCCATGCATGTGTTTGAGGAAAAACAAAAAAAAATTGATGGTTGCCAAAAACAAAAATGCTTCCATATCAAAGTTTATCAGTG |
| | | TCAATGTCAAGAGACTTCTGGTTCGTAGACTCATTTTGGCTTGAGGCCACCAGAAGTGAACTCTGGTTTCTAAATGCAGAAGCAGAGGCA |
| | | CTGGCCGATCATGGAAGATGCAGGGAACTGTTCAAGAGGCCCAAGCCTGGTGCTCAGAAACTTGGCAGGATCAAGCATCTCGCCCAGGAA |
| | | TTCATCCCCTGCTTGTCTAAGCCGGCTGGCTCTCGTGACTGACTCGGAACAACAGAGCAGATGTTTGCGTGGGAGGCAAGCCTCACCCAA |
| | | CATCTGTCCTGCGGCGGGAAGGCCTGGGTGTTCACAGATAGAGCTGGAGTTCCCCGGTGGGTGGCACAGACAATTAGCTGGGGCTGCCTC |
| | | ACATGTAATCTAATTACAGGGGAAACAGGCTCAAACACCGGGTGATAAGCAGCGCAACTGTTTCGGGTGACTCTGTAATTTTTCCTCCAT |
| | | TAATTTTCTCCATAACGCAC |
| 212 | C21orf129 | GTTGCCTGGGATATGCTTATATCAAAAACTTACGTGTCACTTACCTAGCATTTGCATTTCACTGGGCCTCCTAAATTCTGTGTGGTAACC |
| | | GACTGCCACCGGACATGCTGTTTACTTCTCTATCCTCACGCAGCCAGTTGCCACATTCAACATAACACTGCAAATATTGCCGGTGGATCC |
| | | TGACTTCCTCGTGGACCCTACTGTGTCGGGAAAAACAAACAAACGAACCCTGGAAGGAAACACCATGAGT |
| 213 | C2CD2 | TCATAAATATTTCCAAATGTATTCCTATTTGTCTCTACAGAGTCTAACAGACATAAATAGCGAATTGAAGGTTCTGTCTTAAAACCCAGC |
| | | AGAAAGAAAAACAATGACCAGAAAAAAAAAACAATTGTCTTTGGCTTCCCAAGAACAGCATCGGATTTCAACTGGAACCACAGATGGTCC |
| | | GTTGATAGAAGCGACTACTTTTTAGCTCTGGAGGACGACAAAAGGAACCAGCTTCTTCCTGTGGGTGTCACAGCGAGGTCGCCTGGCCAC |
| | | ATCAGGTACCAGAGCGAGCGCCCTCACCTGATAGGCCCTGTACAACCTCAGCCACAGCACTGTCAGGAGGAACACGCGGAACTAGCAACC |
| | | TAGGAGGGTAAAGGCGGAGTTGGGAGGGAACACGAGGCAGGCAGGTCGGCTGGCTGCTGAGCTACAGGCTGCACTCCTAGGACGTCTACG |
| | | TGTAATTGAGAAAAATAAGACAAAAATAACTTACTGTGCAGGCAATTAATTCTGGTTGGCATAGCGATCCTCTTAAGTTAAAGGGAATGA |
| | | GCATGAGATGAAGAGAAGTAAGAGGCAGAAAGAATTATGCAAGAGCAACATCAGAGTGGA |
| 214 | UMODL1 | ACGCCGAGCCGCCTCTGCAGGGGAAACCGAAGCAGATGTGGTGAGATAATACATCCAACCCTGAGTGCTACTCTAACCTGCCAGAGGCGG |
| | | AGGGTTCTCAGTGAGATGAAAGCATTACAGATGCGTTAGATCTAAGGGAGGGGCCTGCAGATGCGCAGCTGGCAGAGAAACCAGGGAGG |
| | | GGCTGAACTGTCAGTCGCGACCACCAGGGATCTGAATCAGTTCACCGACAGCCTTGGGGACATTCACCTTGGGCTCCACAACCTGTCAGA |
| | | AATGCCCCCAAGCCCAAAGGCGTCGAGAGAATGGCCAGGTTGTTTCAGATTGACACATATCCTAATGTACAAGTCAGCCCACACACCCCA |
| | | CGTGCACTGAGCGTCTCTTGTTGTTCACCCCAAATAAACTCTGCCGGAACTGGGGCGGGACTCGCAGGGGCGGAGAAGGGGGGAGACGGG |
| | | CAGAGGGCAGAAGTGGATGGTGAGAAGAGCCAATGGAGGGGCCCCGTGAGAGTGAGCAAGGCTGCACCCCTAACCGACGTCCTGGGGC |
| | | TACTGTACAAACAAAGAACCACAGGCTGGGAGGCTGAACAACAGACCTGCACTCTCTCGCAGCTCGGAGGCTGCAGGTCTGAAATCGAGG |
| | | GGCTGACAGCGCTGGTTTCCTCTGGAGGCTGCGAGGGAGAAACCGTCCCCTGCCTCTCCCAGGCTCTGGGGTGAGCCCTTCCTGGCATC |
| | | CCGGGCTCATTGTAGATGGATCACTCCAATCTCCATGGCTTCTCAGGGCTTCCCTCCATGCACCTCAAATCTCTCTCTCCTTCCTTTTGT |
| | | AAGGATGCCAGTCATTGGATTTAGGTTCACCTTAAATCCAGGATGATCTCATCTAAATTACATCTGCAAAAAGACCCTTTTTCCAAGTAA |
| | | GTTGACATTCACAGGTACCTGGGGTTAGGATTGGACATATCTTTTGCAGGGGTGCAGGGGGCTGCCACTGAGCCCGCTGCACAGGGTGAC |
| | | CTGGGCCAAGGGCCCTTCACTTTCACTTCCTCATTGGCAAGCTGCCCTGTGTTTGGACTGGGTCGAGGCTGTCAACCTTGCTGCCCCTCG |
| | | GAGTCCCCCCTGGTGTCCCCCAAACAGATTCTAAGCTGCTTTCCTGGGGCTGGAGGCCAGGCATTGGGATTTTTTAAAGAGCTTCCCAGC |
| | | AGGTGAGCAGCCTTTCATGGGTATCAGGAGACCTTCCTGGCAAATGTGGTGAAGGTCCTTCCTCCTGAGCGATGCCTTAGACCCAGGAGC |
| | | CCAGGGAGGCTGCTCACCTGATCGTTAGGACAGGAGCAGTGGAAACCTCTGGCCTCAGACCCCCTGGAGGAATCCCTCCCTCTAAGACTC |
| | | TGGGACTGGTGCACGCAAGGAGCTATCGTGAACATTGCTCCCAACTGGCCGCTTGCTTGTCCCCCGGCTCCCCTTGGCCCCAGTGGCGGC |
| | | TTTGCCTGAATTAGAGGGCGTGAGAGCCACCTGTGTCTCAGCACTGCAATTAAAGCAGGAAGCCCTTTCGGAAGCAGCCGTGTGCACCAG |
| | | CCTCCCATGGGTGGAGCAGAGCAAACCACCCACTTCTGCCCTCTGCCCTTCTTCCCTTTTCTCGACACCCTGCGGCCCCCCAGTTTCAGC |
| | | AGAGTTTATTTGGGGTGAAAAACAAGAGATGCTCAGCGCCTGTGGGATGTGTGGGCTGACTCGTACATTAGGATGTGTGTCAATCTGAAA |
| | | TAACCTGGCCGTTATATGGATGCCTTGGGGCTTGGGGGGTTTCTGGCAGTCTGTCGAGCCCGAGGTGAATGTCCCCAAGGCTGCTGGTGA |
| | | ATCAGATCCCTGGCGTTCTCCGTTGGCAGTTCAGCCCAACAGTTTCTCTGCCGGCCGTGCCTCTGCAGGTCCCTCCTCTGATCTGATTGG |
| | | ATTAATATTTGAATCAATAGACTGAGTCAAGCAGAATGTGGGTGGGCCTCATGCAATCAGCTGAAGCCCTGAAAAGAGCAAAAGGGCTGC |
| | | CCCTTCCCCCGAGGAGGAGAGAAC |
| 215 | UMODL1/ | CACATTTCAGAGCTGAGGTGCTGGTGCGGGCAGGTCTCCTGAGCTGGGGGGTCAGCTGTGTGGCCAGTGATGGTGACGCCTCAGGCCGT |
| | C21orf128 | GCATGGCCGGGGAGGCGGCCCTGCCTCTGCACTCTTTTGACTCCATGACTACTGGTGTCTTCGGACGCCAGAGTCGGGGGAGCAACCAT |
| | | GGGGCACCGCCCCTGCCTGGGGAGGCAGCACGAGGCCTGAGCCCAGCTTACAGGGGGACATCCACCCCCGCTGAGAGCCCCACCTTCACG |
| | | GCGAGGATCTGTAGAAGAAGACATTTGATATTACTCGGCAAAAAAAACAAGAAACGAAAACACAAAAAGAGCTCCTCTGAAGAAGAAAAG |
| | | GTATTTGCGCTGTGGTCCACCTAGAAATAATGTTGTTGGCACAACTAGAGCATTCCTCAGTCATTCAGGAGCACTCCCTGCCGGTGCGTC |
| | | CACATGTCCCAACCCCGATAGATGAGGCGCTGTTCGCCCGTGGAGGGGTCAGGTTGTCGTGACCTTATCTTTACCCTTAGGCCGTCCATC |
| | | CCGGGGCCTGGGGTTTCCTGCGCCAGTCACGGTGGGCTGTGTAGGTGGCCATGTGTTCGGTCTTTCCCCAGGAGGTACGTACCATGTGCT |
| | | GGGAGGCCTGGAGGCTGAGCCGCCCCCCGCGCCTATGAGTTGCACCCTCACAGCGGCGGCCAAACCTCCTGC |
| 216 | ABCG1 | CAGGCTTGAGCGGTGACTGGGAGACCCCGGGAATGGAAATGGCGCTCAAATGCTGGTGTGGTGTCCGCAGGGGAACGGCCCGCGGGTG |
| | | TGTGGAGTCTGCGCCCCTGTGGCTTCAGCTGCGTCGGGGGACTGCGGGAATCTTCCAGACTCCAGTTTAAATCAGAGAGGTGTGTCCACG |
| | | AAAAGAGTCAAACTAAAACATT |
| 217 | chr21: | AACGAGACAGTGCAAAAAGCCGCTGCCTGGTGACCTGGCATGCAGACTCGGCCCTCCCACTTGCACGGTGATCCACTGAAGACAACAGCT |
| | 42598300- | GCCTCTGTACTCACGCTCCCCCACACTCCCCTCCTTCCTGCCCTGGTTTCTCCATCCCTAGATGCCATCCCATGCCCCAAACCATCCGCC |
| | 42599600 | AAGCACAATAACCTCGCCCCCACCCACCCCATGAGGTCACTCGAGTTGACAACCAGATAACAGTTTTTGTTTTGTTTTGTTTTGTTTTGT |
| | | TTTGTTTGTTTTTGAGACGGGGTCTCGCTCTGTTGCCCAGGCTGGAGTGCAATGACGTTATCTCGGCTCACCACAACCTCCGCCTCCCGG |
| | | GTTCAAGAGATTCTTCTGCCTCAGCTGCCTGAGTAGCTGGGACTACAGGCGCGTGCCACCATTCTCAGCTAACTTTTGTATTTTTAGTAG |
| | | AGACAGGGTTTCATTATATTGGCCAGGCTGGTCTCGAACTCCTGACCTCTTGATCCGCCCACCTCAGCCTCTCAAAGTGCAGGGATTACA |
| | | GGCGTGAGCCACCGCGCCCAATAGCAATTTGATGACCCATCCCCTCCACTGCTGGGAAAAGGCTGGGCACCGCCCACACTCCATGCAGCT |
| | | CTCTTTCCCTGGCTCGGAATCGCTGCAGGCGCCACAGACCAGACGCGCACTGTTCCCCACTCCTGCTTATCGGCCGCGCGGCATCCCCTT |
| | | GTCGCAGCACTCCAGCATCCATGCAGCCGCGCGGCACCCCGTCTTCGGAGCACTCCAGAATCCATGCAGAGCGCAGCACCCCACATCCAG |
| | | AGCGCTCCAGAATCCATGAAGCACGCGGCACCCCCTCGTCAGAGTGCTCCAGAATCCATGAAGTGCGCAGCACCCCTTAATCGGAGCGCT |
| | | CTAGAACCCGTGCAGCGAGCAGCACCCCACACCCGGAGCGCTCCAGAATCCATGAAGCCAGCAGCACCCCACACCCGGAGTGCTCCAGAA |
| | | TCCACGCAGCACGTGGCATCTCCTCGTCATAGCGTTCTAGAATCCATGCAGCGAGCAGTACCCCACACCGGGAGCGCTCCAGAATCCACG |
| | | CAGCGTCTGGCACATCTTTATCAGAGCGCTCCAGAGTCCATGCAGCCACAGTCCTCCAACGGACCCTGAGATTGTTTCTGCAAAAGGCCA |
| | | TGCCTTCATAAATCTGAAAATTTGGAAAACATCCTTCTACTTATATCCTTACAACCCACCATTCAAGCTGTAGAAGCCTTTCTGGAACCC |
| | | CAAGCAGAAGGATATCCAAAATGTAAAAACGGTGGGGCCT |
| 218 | chr21: | ATAGTGCGACTGTTCCGAAGTCTTTATCACAGTTACTGGTGATGCTTTTTTCCAGATGTCCTCGACGTGCACCCATGAAGGGCTCCACCT |
| | 42910000- | GAGAGTGCCAGGGTCCTCCGTGGGATGGGGCTGGAGGGGGTGCTCTTGCCGTCCTGGGCTCCCAAGCAGCCATAGGAACAATAGGGTGAT |
| | 42911000 | GGGGTCCCAGAGATAGAGGCCAGTGACAGCAGCGCTTTGAACCCCTCACACGGGCACGGGCCCTCTGGCAGGGATGGGCGTCCCGGTCAC |
| | | ACGGAGATGGGGGCTGCTGCTGCCTGCAGGTAGAGGAAGGGACGTGTTTGGCAGTCCTGTGACCCCTGGGCACCTCGCCTCCCCCACGG |
| | | CCGGCTCTGCTTGTAAACAGACAAGTGCACAAGCGCAGCCCGGTGAAGGCACAGCGGTCCCAGGAGGCATCTGGGCTGCACCCCAGCGA |
| | | GCCGCCCATACACGTGGAGATGCCGGCCAAGGCCCTGCAGCACACGGCAGAGGAAGGCGCGATGGGAGCCATGCTGGGCCCGGAAGGTG |
| | | CCGCCGCCCGGAGCTGTAGCCATCACTCCAGCTCTTCTTTTAAGTGTTCCCAGAAATTGTGACCCACCAAAATCTGAGAGCACCCGACAG |
| | | TAAGCCAGAGGACCTTGATGTGAGATCCCAGCACGGTGTGGGGGCGGACTGTGGTGGGTGCTGTCTCGGCCCCCACCCCTTCCACAGGT |
| | | CGGTGTGCACATCCCACGGCGCCTGCTAAGCTGCAGTCTTCTCCAAAGGGGTCACTCTCCGTGGGAAGGGAGCCACCCGCCCCCGGGTG |
| | | ATGTCCCCAGTCAGTGACTGACGACAGTCCCCAGCCGAGGTGAGGGACCAGCTCCTGCATCCCTCACTCCGGGGCTTGCCTGTGGGCCA |
| | | GGGTGGGGGCGAGCCTCAGCAGAGACCGCGTCCCCCTTGCCTGTCCTGCCCTGCCTCCCCTGCCTCCCCCGCGCCTCTGCTGAGCACGC |
| | | CCAGAGGGAGCTGCTTG |
| 219 | PDE9A | CACTTGAAAAGCACAACTCATGGTGCCAAAGCTCTGACACGGACTCCACTGGAGCTGTGGGCAGGGGGTGCCAAGGTACCGAGTTCCAAG |
| | | CCGTTGTTATTTGAGAGCGTGCCCCCCGCCATGAGAGCAGGTGGGGGGACATAAAGTGACACAGGATGGACTGGCCAAAGGCTGAGGAC |
| | | GATCACTTACCTCACAGGATGATGCCACCCCCACGGACAGGCAAGGAGCTCTCACCTTCCCCAGGACCCCAGCTGCCACCAGAGCTCCAG |
| | | ATGGCCCTGGGGGTGTCTGTAAAGCCTGTGACCGTCCACCAGGTGGAGACCAGGCTGGCCAGGGGAGGGAGAGGAAGTGACCACTGGC |
| | | CCTGGCACTGGCTGGCCGGCTCCAGCAGGCCCGAAGGGGAGGGAGGAGCCTGGGTGCACCAGACTCTCTCAATAAGCAGCACCCAGACA |
| | | CTTAACAGATGGAAAGCGGTGGCTTGGAACTCACTTCCAACGAAACAATAGCAC |
| 220 | PDE9A | AGCACCTCCTACCCCACCCTCCCCATTCCTGCCATCCCCAGGGTCCAGGGAGCCCAGATTCCAGGGAAGGGTTGCATTAGCTCCCACTCG |
| | | GAGTCCTGATGCAGCAGAGACAGACAGAGGCCCTGGGAGAAGTGAGCATGAATTATTAAGACAAGACAAGGGTGAGGCCCCAGAGAGGGG |
| | | GTGGCGGAAGGGTCATGTTCATGCAGCGAGAGTTGCTTCGAGCTTGAACCGCGTATCCAGGAGTCAAGCAGATTGCAACTGGCGAGAGGC |
| | | CTTCAGAAATGCCCCGTGAGAGTCCTGTGTGCAGAGCTCCATCTCAGCACACTTCCTGTTCTTTTGGTTCGTCGATTTTTGCATTTTCAG |
| | | TCCCCTGTGATCCATTATTTATAACAGTGGAGATTGGCCTCAGACACTAGCAGTGAGGAAAACAAAAGCGAAGCTACGCAGAAAAATGAC |
| | | AAGAGTGATGAGCACAGCAGTCATGACAAATGAGCCCTGTGCGGAGGCCCGGGATCCGCGCAGATGCCGGCGCGGGGGAAATGGGCCCTG |
| | | AAATCCCACCGTCAGGCCAGGCAGCTCTGAGCGTGACCTGGAGGGCTGTTCAGACGGTCTGGGTAGCCGTGTCCTGCGCATGAACATCCT |
| | | CCGTCGGGAGAGGAATTCCCCACGGATTATCAGAGCTGCTCCCTCCACCCCCCGCCACGTCCCACGCGGGCCACATCAACTCCCTCTGCA |
| | | GCCTCTGGCCAGCGGCTGAGCCCTCCGTGTCTCCCCTCGTTAATGCCTCCTTCACCATCCCCTCCTGAAGTTTCCCCCATTGCATACACG |
| | | CGCTGAGGCCCACCCGGTATCAAGGACTCCCATTGCTTGCGAAAAAGATTCCACCCCTCTTAGAACAGAGACCAGGGCCGCTGTAGCAAA |
| | | TGGCCATAAATGCCACAGCTTAAAACAACAGAAACGGATTATCTCGCAGCTCTGGAGGATGGAGTCCAAAATCTGAATCGCTGGGCTGAA |
| | | ATCCAGGTGTGGGCAGGGCCGCGCTCCCTCTAGAGGCTCCCCCGGAGATTCCCTTCCTTGCCTCTTCCAGCTGCTGGTGGCTGCCAGCAG |
| | | TTTGGGAATTGCGGCCGCATCACACCACCTTTCTGTTTGTTGTTGACATCCCCGCCTCCCCTGCCTGCGGGGTCTTAGATGTCTCTCTCC |
| | | TTCCCACTGAGTTTCACTCCACATTTGAATTGGATTAACTCATGCCATGTTAGGCAAACGTGCCCCTCAAATCCTTCCACTTAACAGACA |
| | | TTTATTGAAGGTTCCTGTGTGCGGGGCCCAAGAGAAGGGA |
| 221 | PDE9A | GAATGTTCAAAGAAAGAGCCCTCCTTGCCTTCCTCTTCTTCCACCCCTGCCCTCTGCAGACTGGGGTTCTGTAGACCCCCAAAGTAAGTC |
| | | CGCCACACCGGAAGGAAGTGAGTTACACAGGGGCCCACATGGGAACCGCTTTTTGTCCTGTCTTGGTGGGAAAATGGCCACGACCCCAGC |
| | | CCAGGCTCTGCCACGCCACA |
| 222 | PDE9A | CCATCTTCCTAGGCCTGCGTTTCCCCCACACCGGGGACTTGTGCTGGAAAGAAAAGCTGCGTTGGCAGCCAGGAGCCGGGGAAACTGTCC |
| | | AGGGAGGCATCCTCTGCGATGAAGGCGGGGCCTCGGCGTGGCCCGTTCCGCGCTCTGTCCAGCCCTGGAGAAGCCCCACCCTCACCGA |
| | | GCTCGAAATACCCCCTCCCTGAGAGCCGAGACTCATGGCCGGGACCCCTTGGACAGAAGATGCGGATGCTAACCCGGCGCTTCCACCACA |
| | | GCCCCGGCGGCACTGGGGAGCGAGCGCGGCCATCCCGCGCGTAGGTGGTGTTTCTCTGCAGGCGCCAGTTTCACCGCGGGCGCCCAGG |
| | | ATCCTCAACGGTTCTGTTGTGATGTGATTCCCCTCTTCGACTTCGTCATTCAGCCTCAGTCCCTCAGTCCCCAAATACCGAAAGGCAGTC |
| | | TTTTTTTTTTTTTTTTGAGACGGAGTTTCACTCTTGTTGCCCAGGCTGGAGTGCAATGGTGCGATCTCGGTTCACTGCAACCTCCGTCTC |
| | | CCTGGCTCAAGCGATTCTCCCGGCTCAGCCTCCCGAGTAGCTGGGATTACAGGCACCTGCCACCACGCCCGGCTAATTTTTTGTATTTTT |
| | | AGTAGAGACGGGGTTTCACCATGTTGGCCAGGATGGTCTGGAACTCCTGATCTCAGGTGATCCACCCGCCTCTGCCTCCCAAAGTGCTGG |
| | | GATTACAGGCGTGAGCCACCGCGCCCGGCCTTTTTTTCTTTTTTCTTTTGAAGTTAATGAACTTGAATTTTATTTTATTTACAGAATAGC |
| | | CCCCATGAGATACTTGAAGACCCGGTGCCAAGCGACAGTGTTGACCCCAGGTGGTCAGTCCTGCCTGGCCCCTTCCGAGGGATGCGCCTT |
| | | CACCATAACCATGTCACGGACAGGCGTGTGGGCAAGGGGGCATCGCTGTATTTTTCACAACTCTTTCCACTGAACACGACAATGACATTT |
| | | TTCACCACCCGTATGCATCAACCAAATGAAAAGATGAGCCTGTGACATTCCCGTGCGTAGAGTTACAGCTTTTCTTTTCAAAACGAACCT |
| | | TCAGTTTGGAGCCGAAGCGGAAGCACGTGGCGTCTGACGTCTCCAGGGAGACCCGCCGCCCTCGCTGCCGCCTCACCGCGCTTCTGTTTT |
| | | GCAGGTAATCTTCAGCAAGTACTGCAACTCCAGCGACATCATGGACCTGTTCTGCATCGCCACCGGCCTGCCTCGGTGAGTGCGCGCTGC |
| | | GGGCTCTGCCCGGTGACGCCACGCGGCCTCCTCGCCTTTTCGGGATGGCTGGGAGGGGCGGGAAGAGGCGCTGAAGGGCCCGAGGCACCG |
| | | GCCTTCTACAAGGGGCTCTTCGAAATCAATCAATGCGCAGAATCCCGAGGGAGGCTCAGCCGCCCTCCGGGCCTCTCTGCCTCCACAGGT |
| | | GATGGCTGTGTCCACAAGGAGGAAACCGTCGGGCTGAATTAAACAGAACCGCCCTCCTAAGAGTGTGGGTTTTTCTGCCGGGCGTGGTGT |
| | | CTCACACCTGTAATCCCAACACTTTGAGAGGCCGAGGTGGGCAGATCACCTGAGGTCAGGAGTTCGAGACCAGC |
| 223 | PDE9A | AGGCAGCAGGGTTAGGACTTCAACATACAACTTTTGGGGGGAGATGTACTTCAGCCCATAACACACCACGTGGGAGGATAACACCGATTT |
| | | CAGAGCTTGCAGAGGAAGCCGCCAGGAACTCCAGTGAGACATCAGCCCCCAGGTGCCTGTCAGGCACGCCGGGCTGTGGGGGGCACCTGG |
| | | GCCCATCTGAGTAACGGAGGCGCATCCGCACTTCCCCCAGGAGTACATTTTTAGAACCCACAGCGCCATAAACCAAAGACAAGGAGACTT |
| | | CCTGGTGCCCCGTCAGCTTCTGGAGGCGACGTTCTCGGCTGACAGCTCTGGCAGCCTCCCCTGTAGGTGAGAGACAGGTAAATGGGACTC |
| | | TTGCTTCCAAAACGGAACAGGGTAAAAATTCTCAAGCGTT |
| 224 | chr21: | TGCTGCACCCCCGCTGCCCTCCCTCCCGCTGGCCGGCAGCACCTTCTCCACCCGGGCCCCTCTGCTCACAGCGCTCCCCGCCCCCGTCT |
| | 43130800- | CCCCGAGGGGCGGGGAGCCAGGACATGGCCCTGAAAGCCTAGCCCTGGCCTTGACCTCCCCAGAGCGCCCTCCCCACCCTCCGCCCTCT |
| | 43131500 | GCCAACCCTGGCCCCTGCCCTGGCCCCGTCCTTGTCCTCTGCTGCTGGCCTTGGGGTCGCGCCCCGCAGACTGGGCTGTGCGTGGGGGT |
| | | CCTGGCGGCCTGTGCCGTCCCACGCCTACGGGGATGGGCGAGGTCCTTCTTGGGGCTTCTCTTACCCACTCTCCAGTCACCTGAGGGCG |
| | | CTGCTTCCCTGCGGCCACCCCAGGTTTCTGTGCAGCCGAAGCCTCTGCCTCTGCGGCCGGGTGATCCCAAGACCCCGGGGTCCAGGGAG |
| | | GCACGGGATCTGCTCCCCCGGTCCCAAATGCACCGGCTGCGCCTTAGGAGGGACGGCCTCCACCCATGGCGCTGGCGCCCAGGGGCCG |
| | | CTCCTCGGACTACAGCACTTGCTCGTCGCCCTGCGCCCTGTTTAGTTCTCATCACCAGCAGCCTGGACTAGGGCCCTGGTCCTTCTGGCC |
| | | TCCTTCCACAGCCCGCTGCACATCTCACCCACTTCCCCGAGGTGCTGTCATTGTTTAGCTGGGCCCCTCAGCCTCCG |
| 225 | U2AF1 | TTAAAGGGGAGTGGTTGTATGAAGAGTTCCTCAGTCAAAGGTGTGCAGCTGGGAAGCCCACCCCACCTAAGAGGGAGGTCTGACAAACTG |
| | | TCCACACTGAACCACTCAGACCTGCATCAGGGCCCCGTTTCTTCCATAAGCCGCCAAGTACAGCCCTGAGTCAACTGAACTCAGGCCTGG |
| | | GAGGCTTCCCAAAGCTGACTTGACTCAGCTTTGAACTGAAATGACCGTACCATGACAACCCTGATGAAAAGCTAAACTGAGCCCAATTAT |
| | | TCAACAGTAAAATTCAGTTGGTCTCACTCA |
| 226 | U2AF1 | TGCTACCAGCTGCTTGGGCTTGGGCAAGTCACCCTAGCTCTCAGATGTCATCTGTAAATGATGACAATGCCAATGTGGCACTGTTCTGAG |
| | | AGTCAGACAGAACGTATGTGTGCTTCACATATGGTGCTCATGAAGTGCTATCATTATCTAAGGAAAACAGAAAACGAAGTTCAGAGTCTC |
| | | TCTAAACGCATGACACCAGACCAACAGGGAGTTTCAAAAAATAGGTCTGAAGTAAATCAATTCTCCTGGTCTCAATACACTGAAAACAAA |
| | | CTATTAGGGGACTGACCGAACCCACCTTAGGAACCACCTTACGTCACCTTCTGTCTCTACTGCAAAACCCTCCCTTAATACTGTTCAAAT |
| | | ACGCTGACAATCCAGATCCATATCCAATGGAACCAGCAATCATGCCTGTGTGCCAGCAATGTCAGGGAGGGAAGCCGATCTCTGATGAAT |
| 227 | chr21: | CAGGTGCCGGCCACCACACCCGGCTAATTTTTGTGTTTTTAGTGGAGACAGGGTTTCGCCATGTTGGCCGGGCTGGTCTCAAACTCCTGA |
| | 43446600- | CCTCATGTGATCCACCCGCCTCGGCCTTCCAAAGTGCTGGGATTACAAGTGTAAGCCACTGCGCCCGGCCAAGAGTGAAGTTCTGATAGC |
| | 43447600 | TGGGGTAAGAAAGGCCGTGGGAACAGCCGGTTTCAGACACGCTGGGTCTAAGACGCTGCGTCTGGCGCTGCTCGGCATCCAATGGGAGCC |
| | | GTGGAGAAGCCAGGCGAGTGCGTAGGGCGGAGCCAGCGCACAGGAAATAGGACGTGATGAGGTCAACCGGCTGGTCCAAGTGTGGACGG |
| | | AAGTAGAGGATGCAAGCACCGAGCCCCGGGGCCCCCAGCATTGGCGGGGAGGAGCTCGCGGTGCGGGAGAAGCAGGGGACCGCGCAT |
| | | CCTGGAGACCAGGTGGAGCCAGTGCGCCCGGAAGGGGCGTGGCCCGCTGACAGCCGCCCAGGAGGCCGGGGGAGGCCTGGAGCCGAG |
| | | GGCCGCGCGTGGCAATGTGGAGAGACATTTTGGTGGAGTCATGGGGCCACAGCCTGATTGGTGAGAACAGGAAGGGAAATTGCAGATGG |
| | | GCCTGGGCCCCCTGGCTCCCGCATACTCCAGGACCAGGGCTGAGTCATCGTTCACCGTGTGTGACCAGGGCCCCGTGTGGCCGGCTGTC |
| | | ACTCGGTATCCAGTTACCCTGGGCAGACCACTGGCGGCACCCCCCAGCCAGAGGCCGCAGCAACACACACGCCTGCAGGCGACCAGGCC |
| | | GGACTGCATGCCCCGTGGGGGAACTGAGGGCGTTTCAGTAACAGAGTGTTAGGGGACACGGGTTGGGTGGCTTGGAAAGGGCCTAAGGT |
| | | GGGGTTTGTTTTAGATTGGGGTGGTGAGGGCGCAGGGGCCCGGTAGGATTCTCTAACAGGGCAGCAGCCACTCATTTAGCAACAGGAGAG |
| | | GCGTCCAGCGTTTCGTGGGCT |
| 228 | CRYAA | ACCCAACCACAGGCCTCCTCTCTGAGCCACGGGTGAGCGGTGCAGGTTCTGCTGTTCTGGAGGGCCTGAGTCCCACCCAGCACCTCATAA |
| | | ACAGGGTCCTCCCCAGGGCTGCTGCAGTAGGCATCAACGCCAGGGTGCAAAATGCCTCAGGGAGCCAAGGCTGAGCCAGGGGAGTGAGA |
| | | AGGAGCATGTGGAAGTGCGTTTTGGAGAGGCAGCTGCGCAGGCTGTCAGCAGGCTCCGGCCGCTTCTATAGACAGCATGACACCAAGGG |
| | | CAGTGACCTCATTCCACAGGCTGAGTCCAGCCAGCCAGCCAAGCATCACCAGCCAGACGATTGACCCTAACGGACCAACCAACCCGTAAC |
| | | GACCCCTCCTACCATAACCAGTAGCCAGCCAGCCCATAACCAGCCAACTTATCTATAACCAGCCACCTGACCATAGCCAAACAACCAGCC |
| | | GGCCCACCAGTAGCATTCAGCCCCTCAGCTGGCCCTGAGGGTTTGGAGACAGGTCGAGGGTCATGCCTGTCTGTCCAGGAGACAGTCACA |
| | | GGCCCCCGAAAGCTCTGCCCCACTTGGTGTGTGGGAGAAGAGGCCGGCAGGTGACCGAAGCATCTCTGTTCTGATAACCGGGACCCGCCC |
| | | TGTCTCTGCCAACCCCAGCAGGGACGGCACCCTCTGGGCAGCTCCACATGGCACGTTTGGATTTCAGGTTCGATCCGACCGGGACAAGTT |
| | | CGTCATCTTCCTCGATGTGAAGCACTTCTCCCCGGAGGACCTCACCGTGAAGGTGCAGGACGACTTTGTGGAGATCCACGGAAAGCACAA |
| | | CGAGCGCCAGGTGAGCCCAGGCACTGAGAGGTGGGAGAGGGGGGCGAGTTGGGCGCGAGGACAAGGGGGTCACGGCGGGCACGACCG |
| | | GGCCTGCACACCTGCACCATGCCTTCAACCCTGGGAGAGGGACGCTCTCCAGGGGACCCCGAATCAGGCCTGGCTTTTCCCCAAGGGAG |
| | | GGGCCGTGCCCACCTGAGCACAGCCAGCCCCTCCCGGTGACAGAGGTCACCATTCCCGAGCTAATGTGGCTCAGGGATCCAGGTTAGGG |
| | | TCCCTTCCCGGGCTGCACCCAGCCGTCGCCAGCTCCATCCCTGTCACCTGGATGCCAGGGTGGTCTTAGAAAGAACCCCAGGAAGTGGGA |
| | | GTGCCCCGGGTGGCCGCCTCCTAGCCAGTGTACATCTTCACATGAACCCTACCTGAGGAAGCCAGTCCCCGACGGCATAGCTGCATCCGC |
| | | TTGGAATGCTTTACAGGCATTGACACCTTCGCCTCACAGCAGCACTTTGGAACCAGTGTCCTCATTATTCCAGGGCACGGCTGGGGAACA |
| | | AGGGGGTCCTCAGCCTGCTGGGTCCCACAGCTAGTACCGGGCAGGTGGACGGGAGCTTCTCCCCACAGTCACCCTGATGCCCCGCTCTTG |
| | | CTCGGCTGGAGGCCTCGGATCTCCGTGGTGTTGAGGGAGCCGGGGCACTGGAGCCCTGGTGACCTGCATCTCCTGGCGGAGCCGGGAA |
| | | GAGCTCATGGACTGTCACAGATGGACAGTGCCCCGCGGGGGCTGGAGAGCAGAGTGGGGCTGGAAGGTGGAACTCTTAGCCAAAGTCTT |
| | | GGTTTCTTTTGGCCAGGGTCCTCTTTCAATGGCTGGAGAAGGTGGTGCTGGGGGGTGAACGCTGACCTCCTCATGTGCTGCCCCTCCCTC |
| | | GCCTGGGCCCGGTAAAGCCCCCACGTAGCCCCAGCCAGCCTGGAACATGCTTCCTGAGCTCCCAGCTCTTGGTCTTTGCACCCAGTGGAG |
| | | GAGGAGGTCAGCCCAGGGAGCTGAGTCTGCGGTTTAGGGCGTCCAGGGGACGTGGAAGCATGTGGGTCGTCTGGCCACATTAGGTAGGG |
| | | CTGCAGAGACCTGGGCTAGAGCAGTCCTGCGGGGTCTGGAAGGGGAAGACTGGCTGAGGTGCGGGGCCTGGTCTGGAATGATCCTGCGA |
| | | TTTTGGAGTGAAGCCATGGAGCGGGAAGAGACAACCCCCCGCGGGGAATAGCCCGGCAAGTGGCCACGAGGCCAGGCTGAGGTCCAGA |
| | | GAAGCAGGGGCATGAATCCATAAATCCCAGGGGGCCTGGCCATGGGATGTGCTGGCTGCACCCGGCCCCTGTGAGAGCCCCCGCAGGCT |
| | | GGCCCCCTTCTGCAGTCAGTGGGGCTGGGGCAGCTTCTCTGGCATGGGGCGAGGCAGCCGCCTGCACAGTGGCCCCCCTGACTGTGCG |
| | | CCCCCACCCTCTCCAGGACGACCACGGCTACATTTCCCGTGAGTTCCACCGCCGCTACCGCCTGCCGTCCAACGTGGACCAGTCGGCCCT |
| | | CTCTTGCTCCCTGTCTGCCGATGGCATGCTGACCTTCTGTGGCCCCAAGATCCAGACTGGCCTGGATGCCACCCACGCCGAGCGAGCCAT |
| | | CCCCGTGTCGCGGGAGGAGAAGCCCACCTCGGCTCCCTCGTCCTAAGCAGGCATTGCCTCGGCTGGCTCCCCTGCAGCCCTGGCCCATCA |
| | | TGGGGGGAGCACCCTGAGGGCGGGGTGTCTGTCTTCCTTTGCTTCCCTTTTTTCCTTTCCACCTTCTCACATGGAATGAGGGTTTGAGAG |
| | | AGCAGCCAGGAGAGCTTAGGGTCTCAGGGTGTCCCAGACCCCGACACCGGCCAGTGGCGGAAGTGACCGCACCTCACACTCCTTTAGATA |
| | | GCAGCCTGGCTCCCCTGGGGTGCAGGCGCCTCAACTCTGCTGAGGGTCCAGAAGGAGGGGGTGACCTCCGGCCAGGTGCCTCCTGACA |
| | | CACCTGCAGCCTCCCTCCGCGGCGGGCCCTGCCCACACCTCCTGGGGCGCGTGAGGCCCGTGGGGCCGGGGCTTCTGTGCACCTGGGC |
| | | TCTCGCGGCCTCTTCTCTCAGACCGTCTTCCTCCAACCCCTCTATGTAGTGCCGCTCTTGGGGACATGGGTCGCCCATGAGAGCGCAGCC |
| | | CGCGGCAATCAATAAACAGCAGGTGATACAAGCAACCCGCCGTCTGCTGGTGCTGTCTCCATCAGGGGCGCGAGGGGCAGGAGGGCGGC |
| | | GCCGGGAGGGAGGACAGCGGGGTCTCCTGCTCGCGTTGGACCCGGTGGCCTCGGAACGATGG |
| 229 | chr21: | TTTTTGTGTTTTTAGTAGAGATGGGATTTCACCATGTTGGCCAGGCTGGTCTCAAACTCCTGGCCTCATGCAATCCTCCTGCCTCAGTAG |
| | 43545000- | TAGTAGTTGGGATTACAGGTGTGAGCTGCCATGCCCAGCTGCAGGTGCGGAAGCTGGGGGCCTCAGAGACTGTGGACTCCTGGCCGGTGA |
| | 43546000 | GGAGCGGCATGGGCCGGGAGAGCTGACTCTTCAGCGGGACTGAGGTGGCTGGAGCGTGACCCTTTCCTGAGGGCAAACAGGGAGGGCCT |
| | | TGGAGCCCGGCGCTCAGGACAGGCCCCTGCTGGCCCGGCAGCCTGAGCTTCCACACTTTTCCAGGGCGTCTCGAGTTCGCCCACAGAGC |
| | | TGTTGTTTCAGGATAAAAAATGCCCTTGTATTCCACGTTCCAGTTCAGAGGCCCGTCTGTTCCCAAGAGCGGAGGCGTCAGCCGCATGAG |
| | | TCCCACCGGAAGCCGGGTTGCCGGGTCCCCGTCCCTGCCCTGCAGACGACGCATTCCGGAGCCCCCTTGGGAAGCTGCCTGGCTCTCCCA |
| | | GGCCTGGCTGCCTTCGCACGAGGGCTCCGAGGCATGCTCATCCTACGTGACTGCCCGAGTGTGCACACGCCTGGCCGTGTGTGGGCGTG |
| | | TGCCTGGGGCCCGAGCTCAGGAGCAAGGCCTGCGTGGACCTGTTGTCTGAAACAAGCCAGTAGACAGCTGCGTCAATGCAGGCAAGCTG |
| | | AACAGGGCTGCTTTTTCAGCCTGACAACCCCAGGGGCTGAACAGGAGCTGGGGGAGGAGCAAGGGGCCGTTCCCCTGCCCCACAGCACA |
| | | GCACACGACCCCGCCTTGGAACCTGGGGCCCGGGGTGAATCGAGGGTCCTGGAGCAAGAGGGGCTGCTCCACAGGAGAGCCTGTCCCG |
| | | CCACCCCTCAGCCACCAGATTCGGGGCTGCTGGACTTGTTCTCAAACCTGCACAGTGAGTGACAGCTGCTGAGACGGAGGTCTCAGGCA |
| | | GTGCAGGTGAATCAGCAT |
| 230 | chr21: | TCCTTATTTTTTAGTTCTCAAGCCCTGTAGGGTGTTTTCGGTCGCAGTTGTTTGGGCTGTGGTCCTGACCCTCCTGAGTTCCAGTGGCTC |
| | 43606000- | TGTTCAGGAGAGCTGCCTGGGGCCGGGACTTCTGAAACACACACTGAGCCACAGGCCGGCCCGGCGGCTTGGGTTCACCGCCGCCTCTTT |
| | 43606500 | GTGTGTGATGTCCTGGGATAGGCCCGTGCACGTTCAGATGACACTGTACATATAAATAACTTGTAGCCGAGAACAGGATGGGGCGGGGAG |
| | | GAGGGGAGGGCAGAACGTACCACAGCAGCAGAAGTCACTGTGGATGCCTTCGTAAGTTGCATGGAAGGTTTTTAAACCTAGCCCTGCCGA |
| | | GCAGCCCTCTCCTGGTCCGGGAGAACGATGGGGAGAGAGCTGGCGTTCAGCTTTCATCACTGGAGCCGTTCCTTCTTCCGGCCCCCCGAG |
| | | GGCCTGTCCATGATCACACTTTGTCTTGTTTCGGGGGTGGCCCCTGTGAC |
| 231 | chr21: | CAAGCCTGTGGTAGGGACCAGGTCAGAGTAAACAGGAAGACAGCTTTCGGCCAGGCGGTGCACCTCGGTGCCGGTGAGTGTGAGCGTGT |
| | 43643000- | GTGCGTGTGCACGTGTGCAGATGTGTGTGGACGCTCCCTTCTCCGCAGCAGCTCCTGACCCCCTGCAGGTGACCCTCAGCCAGCCCCAG |
| | 43644300 | GGCTGCCCCCACTCTCCCCTGTGGACACCTACCTCATTTGGGGTGAAGTGGGGGGACTGGGGTGTGAGGGGTGCTTTGGGGGGCACACT |
| | | TCGACCCCTCTCTCTGCAGGCCAAGTCCTGAGGCTCAGTTTCCTCCTCTGTGCCCCGGCGACGTGGTGCAGGCCTCGCGAGTGACGTGAG |
| | | GGTTCATGACCCAGGTGTGGGCAGCCAGCCCTTCACGGGAGGCCACCCACCTGGCCACAGTGCCTGGGAATTTAGGTCGGGCACTGCCG |
| | | ATATGTCGCCTTCCACAAGGCGGGCCCGGGCCTCTGCTGACCGTGCACCGGTCCTGGGGCTGGGTAATTCTGCAGCAGCAGCGCAGCCC |
| | | ATGCCGGGGAATTTGCGGGCAGAGGAGACAGTGAGGCCCGCGTTCTGTGCGGGAACTCCCGAGCTCACAGAGCCCAAGACCACACGGCT |
| | | GCATCTGCTTGGCTGACTGGGCCAGGCCCACGCGTAGTAACCCGGACGTCTCTCTCTCACAGTCCCCTTGCGTCTGGCCAGGGAGCTGCC |
| | | AGGCTGCACCCCGCGGTGGGGATCGGGAGAGGGGCAGTGTCGCCCATCCCCGGAAGGCTGAGCCTGGTGCAGCCAGGGAGTGAGGGG |
| | | GCGGGAAGCCGGGGTGCTGCCCTGAGGGTGCCCCGACACGCTCTCCTGGGGCCCTGAGCGGCTGCCACGTGCGTCCAGGGTTCTGGCC |
| | | ACAGGGTGGGCAGGGGCCCTGTGCTCCTCACTGGAGGCCCCTGAGGCTCTGGAACTGAGACCATCCACCCGCCGGCCCCCTCTCGCCGG |
| | | CTCCGGCACCCCTGCCTACTGTGACTTCCTGCCCCGGACTCGCTCTGCCAGCTTGGGGCAAACCACTTCCCTCTGGGGTTTTCACTTCCC |
| | | TCTTTCCCAAGTGGGGAAAGACCACCTGTCCCCGACCCAGAAAGGGCCCCTGCCCGAGGGCAGCAGCAGTGCCAGGCTGGCATGTGAGG |
| | | CTTGGGGCAGGCCCGGCCCCCAGAGGCACAGGGCGATGCTCTGTGGGACGCTGTGTCGTTTCTAAGTACAAGGTCAGGAGAGGAGCCCC |
| | | CTGACCCCGGAGGGGAGGAGAGGCAGGGCAGGAAACCGCCACCATCTCAGCCCA |
| 232 | C21orf125 | GCCCACTGTGGGTGTGCCCGTGTGTGTGGCTGTGAGGCGTGAGTGCAGGCGTGAAGTGTCTGGGAGTGGGAGCGGGCATGAGTGTGTGC |
| | | CACGGGCCTGCTGTTGGGTCCTTGGAGGCCACGGTTGCCCCTGAAGGGACTGCAAGCTCTTTTTTGATTTGTAGTTATTTGAGAAGTCTA |
| | | TACAGGAAGAAAATTAAACCG |
| 233 | C21orf125 | AGCGCCCAGCGCAGGGCCGGGACCCAGAGTGGACTCTACCGTGGGGCTGCCTCAAAGAAATCTCAGCAAACACAGGAAGCCAGCCCACCC |
| | | GTGCAGCCATGGGGCCAGGAAGCCCGCCCTTTACCAAGTCATTTGGGCATTTTTTCTCTGTGCTAACAGCCCAGATGGAGCCATAGCCTC |
| | | AACCTCTGTGTTCTGATAACACCAAGCTGGGACGCCGGAGCCATGCAGGGGACAGTGCCCGGCCTGAGGCTGCAGCCTGGGTCTGGATG |
| | | CCTTTCTAATTCAGGGCCTCCTCATGGCCTGGTTCCATAAATGGTCAAATGCAGCCTGACAGCGCAGCCTCCTATCAGCGCTGGGCTCCG |
| | | TACCGCCACACAGCCCACATACCCCGTTCCCCAGGAGACGCCCGCAGGTGGGCAGCGTCACTCCCACCCGCCGAGCACACGCTGTCCCCG |
| | | TCTCGTGTCCCGAGGAGCCGGAAGCAGCTGCTTCCTCCCAGCCTGAAAGCTGCACCTCGGGCTGCACTCGGCTCCCCGAACCCGCCCTC |
| | | CGCTGCCCTGCAATTCGCCAAGGGAGCTACCCTTCCCATATAAAAATTTCACCTCCATTTCCTTGTAGAGAAGAAACATTTCTGACAGCA |
| | | AGGAAGATTCTAATTTGAAAAGCAAGTGATTCATCTCCCGGTGCCAAACAGCAGACGCAGGCGTTACCAGTCTGGGTGGGGCGCCCGAGC |
| | | TGGGGACCTGGGGTCCTCTGGGAGGGGCAAGAAGGCAGCGATGCTGGCCCCCGCCTCCATCTGCCCATCCCATCTGCTTCCACACACCGC |
| | | CCTGCCGTAGCTGCTTGCAGCCCTTCTCTGTCAGTTTCTCCATCTTTTGGTTTGGTGATAAATGAGAGTTCCCATCGGGTGTGCCACCCT |
| | | CTGTGTGACGGGGAGCAGAGAAGACCCTGCGTCCAAGTCCTCCTGGGGGAAGAGCGAAGATGCTGGGACCAGCCCCAGCTGTCAGGGGGT |
| | | CTCCAATCCCAG |
| 234 | HSF2BP | GGAACGGAGAGCCGCCAGGCCCAAACCTCCCAGAATTTGCGCAGTATTCTCGGCCTAGAGAGCGAGGAGTGGCCTTGGCGAGGTCCCTCT |
| | | TTGGCTCTTCTGGCTTAGCCGGGGTTTTAAACTTGTTATCTGCAAAGCAGAAGGAAAGTCAGCCCCTGATGTAAGTGTCAAGTAAAATAA |
| | | ATCGGATGGGTCCTTTCCTGTTTGGCGAGGAATGCTACACTAAGGGGGACTGCGTTCAAATGGGCAGTCTTTGCTGGAAACCTCGCCTCC |
| | | GCGCGCCTTCCCTCGCTCGGATTCAGGCGCTTTTACGTTAAGGGTTGAATTTTTGTGTCAACAGGCACCTCGGGAGGTCGCCTAGACAAC |
| | | TGAGCGGAGCAACTGAGATAACCCCCGCTACGTGTGGAGTGACCTAGTCCATTAACTTGCCCCAGCACGCCCGCTGAGTCCGCAAAATAT |
| | | AGGATGGCCTCGGGTTTTAGATGAACCCAAAGCTAAGATTTCTTCCCTCTCTGGAATTAGCAAGCAGCCCGCCCTGCCCAACTCCCCTGG |
| | | AAGCGCGCGTGCTCGCCAGGCCTCGGGACGCCTGCGCGGGCGCCCTTGCACTGGCACCAGGGCTCCGGGGTAGGGGCGCACCGATCTGCC |
| | | CAAGCCTCTGCAGGCACTGGAGGAAGGCGAGCCCTCCACCCGCTCAACAGGCCCCAGTGCCGGCCTTTCCTTCCAGTCTCAACTCCACCC |
| | | GGGGGCCCGGGGGCTCCACAGTTAAAAACTCCACGCCACGGAGATCGCAGGTAAGCTGCTGGCTCAACGAGGTGTGCTAAATGGGATTAA |
| | | AGATCCTGGACCGTGGCCAGGCGCGGCGGCTCAAGCCTGTAATCCCAGCGATCAGGGAGGCCGCCGCGGGAGGATTGCTTGAGCCCAGGA |
| | | GTTTGAGACCAGCTTGGGCAACATAGCGAGACACCGTCTCTACAAAAAAATAACAAATAGTGGGGCGTGATGGCGCGCGCCTGTAGTCTC |
| | | AGCTACTTGGGCGGTCGAGATGGGAGGATCGATCGAGTCTGGGAGGTCGAGGCTGCAGTGAGCCAGGATCACCGCCAAGATCGCGCCACT |
| | | GCATTCCAGCCTGGGCGACAGAGGGAGACCCTGTCTCAAAAACAAACAAAAAATCCTAGACCGTTTACAAACAGCCTTCCGTCTCTTCCT |
| | | GGTCAAGTCCTAACCCTGGCTAACCTCGCCGTCTACAGCCTGAATTTTGGCAACCGAAAGGCAGCGCCGGCGCCACGTGCACACGGGCTG |
| | | GGCCGCTCCGCCAGCTGCCAGGGCCACTGCCGCGCTCACT |
| 235 | AGPAT3 | CGCACACACAGCACAGACGCCTGCATCTTCCCATGCGTGGTTTCTGCTCTTGCCTCTCTGGGTTTTTGTTTCACTTCGGTCGAGTTTTTG |
| | | GTGGTGTTGAGCGGATAGCCGGGGAAGTTGGAGTCTTGTTTGTGGCCGCCTCGTGCTCGTGTCTGTATCTAAGATCCTCAGGCTGCTCCT |
| | | TTTTGGGTAAGGTCTGTTGCTTCTCTAGGAACAGTGACGGTGGCAGAGCCCGTGGCCCCTCTCTCCTGTCCCAGAGCCAAGCTGTTTCCT |
| | | CTCCCCACTCCCGGGCACCCTGCGGGCAAG |
| 236 | chr21: | CACAGCCCAGCTTCAAGCCTGGCCGACCAGGGGTTTGGCATGAAGACCCCGGCAGGGCTGGGGCTGTGCTGGAATCCACCCGGAAGTTT |
| | 44446500- | CCTGCCCCTTGGGCTGCCCACCAGGTCCCCTTTCTGCTCTGATCAAGCTGGACAAAACGTCGTGGGGCCACAGCACAGGGGGCCAACGC |
| | 44447500 | AAGCTGGGATCGTCAGACGTTAGGAAATCCCAAGGAAGAAGAGAAAGGGGACACATTCGGGAGACGTCGGCACACGCTCGAAGCAGCGG |
| | | ACAGGCACCTCTCTGTGGACAAGGCAGACTGGGCGGCCGAGATTCCGCATAGATGCCTGCTTCCTCCACGACCTCCACGTGTGGCTGGCC |
| | | CAGTCCGGGTCCCCCTCACCTCCTCTGTCTGTCTTGGTGGCCTCACGCCGTGGGCTGTGATGCCGGCTACGCTGCTTGGGTGGCCAAGG |
| | | GTCTGAGCTGCAAGACGCCCAGCCTGGGTCTCTCCCGAGCTCTCCCACGTCCTGTCTGCTCCTCCTCCGAGCTCCCGGTTGACTCTCACG |
| | | ACTGCACCAGCCTCTCCCCCAGGAAGGCGTGGAAACAACCTCCTTCTCCCAGGCCCGCTCTGCCTCCTGCGTTTCAAGGCAAATCCGTTC |
| | | CTCCAGGAGATGATGCAACCACATCCTGTTGGAGCCCAGAGAAGTGCGGATGCAGCCCGGGGCTCTTTCTTTCCTAGAACCCTGCCTGGG |
| | | AGTGGCTTCCCTGAACTAAGGACAGAGACTTTGTCTTCGTTGCCTCTCGGCCTGTGGGCACTGAGCATACAGTAGGTGCTCAGTAAATGC |
| | | TTGCAGGCCGATGCCCAGAGCCATTAGCCCTCATCATGGTGAGCTCGGCAGCCGGTGTTGGGGCTGGGCTGGGCCTAGGTGTGCGTGGGG |
| | | GCGGTGCTGGTCTGCTTTGCTGGGAGCCATGGACACCGGAGGAACAGGGCCCCATCAGTGCGGTCAGAGTGCAAACTCGGAGCGTCCTT |
| | | CTCTGGAAAACGAAT |
| 237 | TRPM2 | GGGAGGGGGCGTGGCCAGCAGGCAGCTGGGTGGGGCTGAGCCAGGGCGATCCGACCCCGAACCGGAGCTTTTAGCACTTTGAGTCCCT |
| | | GTACTCAGAGGTCTCCTGCAGCCGGGAATCCCACTGTGCTGTGGTCCCTGGCAGCCAGCACCCACCCCCAGCTTCTCCGTCAAGGTTGAG |
| | | GACGGAGCACTCCTGCCTCTGATTAACTGGACGCAGGAGAAGCAGTTGCTTTAATCCGGAGCCTTGAGTTGGGACAGATAATGAGTCATT |
| | | CAACCAGATTTTCCAAGGACACACTAACTTTGGTATGATGCGTGTGTGCCCCTGAATCCACGTGGTCAGGAAAGCCCAGGGAACACTGGC |
| | | CTGTGACTCACTGAGCAGGTTCCCTTGTTACCCCGAGGGGTGATTTACTCCTCTGACAGTGACACGGACACTGTGCGTCCATTCCCCGGG |
| | | CGGGCAGAGGACACTCCCAGATGCCCACGAGGGGCCCAGCAAGCACTGGCCA |
| 238 | C21orf29 | CTGCAGGACCTGCTCGTTCACAGATGTTCTCCTAGAAGCAGAAGCTGTTTCTTGTTGCAAACAAATTTGCTGTGTCCTGTCTTAGGAGTC |
| | | TCACCTGAATTTACCAAGGATGCATCTGTGCTTGGGGATGGCTCGGTTTGAGGGGTCTGAGGAGCGGCTCCCCTGGATCCTTTCCTCCCC |
| | | AGGAGCCCACCTGCCGAGCTGTCAGCGTCAGCCCCACATCTCAAGATGAGGAAATGGAGGTCGAAGCCATGCACACGCAGGCGTCCTGCT |
| | | GACATGCAGGCCAGGCGGGTGCCTCTGTATTCAGCAGCCTCAGGGCTGTGGCCAGTTCAGGCAGCAGAGGGGCCTCATCCCGGTGCTTCC |
| | | CTGCAGGCAGTTGTGGGGCCGGCCTGCAGCAGGGGCTCAGACAGGGCCTTGGGAGAGGGAGGGATCACAGAGGTGTCCAGTGACAGGC |
| | | AGGGCGGGCAGAGCCCATGGGGCCTTGGGCTCCTCACTCCTTCGGTCAGTCAGGGTGACATCTGGAGCCACCTCCATTAATGGTGGGTTA |
| | | TGATTTGGTTCCCATGCAGCCCGTGCCAGCTCGCTGGGAGGAGGACGAGGACGCCTGTGATC |
| 239 | C21orf29 | AAGAGGAAATTCCCACCTAATAAATTTTGGTCAGACCGGTTGATCTCAAAACCCTGTCTCCTGATAAGATGTTATCAATGACAATGGTGC |
| | | CCGAAACTTCATTAGCAATTTTAATTTCGCCTTGGAGCTGTGGTCCTGTGATCTCGCCCTGCCTCCACTGGCCTTGTGATATTCTATTAC |
| | | CCTGTTAAGTACTTGCTGTCTGTCACCCACACCTATTCGCACACTCCTTCCCCTTTTGAAACTCCCTAATAAAAACTTGCTGGTTTTTGC |
| | | GGCTTGTGGGGCATCACAGATCCTACCAACGTGTGATGTCTCCCCCGGACGCCCAGCTTTAAAATTTCTCTCTTTTGTACTCTGTCCCTT |
| | | TATTTCTCAAGCCAGTCGATGCTTAGGAAAATAGAAAAGAACCTACGTGATTATCGGGGCAGGTCCCCCGATAACCCCCAGCTGCAGATC |
| | | GAGGCCTAGTGCGAGCACAGGTCCCCCCAGACCCTTCCCAGTGCCCACCAACCGGCGGCCTAGGCCAGGTAGAACTGGCAGCGCCTCCCC |
| | | TGCTGCAACACCAGGCTCTGGTAGAAACTTCAGAAAACATGCACCGGCAAAACCAAGGAAGGGTGGCTGCGTCCCGGGTTCTTCCGCGCA |
| | | GCTGTGTGTACACGCATGCACACACCCACACGCACACACCCACGTGCACACCCCCATGCACACGCACCCACTTGCACGCCCATGCACGCA |
| | | CACACGCGCGTGCACCCATGCGCACGCACCCATGCACACACACGCGCGCACACACCCACGTGCGCACCCACATGTACACACCCACGTGCA |
| | | CACACCCACGCGTACACACCCACGCGCACACACCGCTGTCCCCAGCCGTGCAGAACGATCCTCCCTGAGTCCCCGGCTCCGACCCACACG |
| | | CAGCACTCGCTAAACGCTTCCCACGCAGTCGTTTTGCTGGGTTGCGCTTCACCCACTTCTCAGAGGGGGCGGCCGAGGCAGAGGTGTCGG |
| | | GGATCGAGCAGCTCCGGGCCTCAGGGGTCGCCCCGCCACCGTTTTCCTTTCCCAGATGCTGGGACGGGGGCAGGGAGGGGCTCCCCAGGC |
| | | TGAACCCGACTAGGTCACCCTAGAAGCGAGGCGAGCTTCTCTTCTGTTTTTCTTCGGCGCCCCTGAGCCCCTGACAGTGCCCAAGCTGCC |
| | | CATGGGATTGGATTCGCCAGAGCCTCCTACGCAGACCCCACCCAGGGCCAAAGCCAACCCCAAGCCCCACCACCTTGGTGGTGTGGGATG |
| | | AAAAGTGAGCCATCGAGAGATGGGGTCCCCCCACCCCCAACCCCTCCAAGGACAAAGGCGGGCTGGGAAGCACCCGCTTTCACGTCCGCC |
| | | CCTGCCCGGCTTTCCTAGCGGAATTGGCGCCGGCATCAGTTGGGGGTTGTGGGATCAGTGAGGAATCCCGTGGGGTCGCCTCCATTTATC |
| | | AGTTGTGTGGGGTTGGGCGAGCACCCCTAGCCCCAGCCCAGGCGATCAGGGCGCGAAGCCCACTGGACGCGGATTTGGGATTAGGACGGG |
| | | GGTGACAGCCAGGAGGACCGCACCTGCCCTCCCCACTCCTGCCGCTCCACCCCTGCCCCCACCGCAACACCAAGGTCTCCACCAGGAAGA |
| | | TGGGGGTGGGGAAAGGACGCGGGGTGGGGGGGGGTGCGGGGAGAGAGGACACAGGGTCGGAAGGGTGAGGGGTAGTGGCAGAGGCGG |
| | | AGGCCGAGGCCACGCAGCTGCGGGGCGCAGGGAGGGGCAGAGGAGGGGCGTTCAGATGGGAACCTAGTCCAGACCCGTCGGGGCCCT |
| | | CGTGTGCGGCTCGTTATCCTGGAACCAGAGAGGCTGGAGACCCTTGGCTTGTCTGGAGCGGAACCGTAGTGTCCAATAGAGTGTGTGGGG |
| | | CTCAGCCCTAAAGCTAAACATTCTTTATTTCCTGATGACCATGGGGGCGGAGCGGGGGAAAAGCCCTGGCCTTATAGTTTAGAATTTTAT |
| | | AAAAGGAAAGGCGTGGCCACTGACAATTTGCGCTTCAGGAGTCCCAGAGTGACCGCCTGGCTCGGAGCAGGGAATGAGGGGGTCCTTAAC |
| | | TCTGAGATTTGTTTTCTGAGAGACAAAGGTGATGGGTGAGGCGGCTAAGCCTCTGATTCTCTATAGGTGGCGGTCATTCATTTCAGAACA |
| | | TGAATGGATTCAGTAAATAAACATGATAGAAAAATGCCACAAGCCCTAGGCCCATTGGAGTGGACTGGACAGTCTGTTCCCAGTGTGTCC |
| | | CTCAGCCTCGGTCCCCCACCCTTCCCGGAGCCCTGGGGGTCACACACATCCCTCCTGGCTGCCTAGCCTGTGCCCCCCGATTCCCCCCCT |
| | | CCCCGCCCCGCGCGTGCACACACACACACACACACACACACACACACACACACACCACACAGCACGAGGCGACAGAGATATGAGAGAGAG |
| | | CGAGCGAGAGAGGACGGGAGAGAGAGGGAGTGCAAGTGTGCGCTGGGGGTAACCCGTGCATGCATGCATTGGGGGTAACAGGCTGGAGCT |
| | | CAGATCCCTCCCCCAGCCCCCAGCAGGGGGGACTGCAGGCTCCTGGTCTGAGTGGGGAGCTGGGCCCCCTGGACAGAGGACTGGGCTG |
| | | CGGGGTCAGGAATGGGCACACTTCCTAACTGCAGGACACTCTAAGGGCTTTGGTCATGCACACGCAGCCAAGAGAAGGTGTCGCTGGCAC |
| | | ACAGCCTTCCAGGAGCGGACTTGGAGACCTCGCCAAGGACCAGGACTCCCCAGCACTCACACTCCCTTAGGCGCTGAAGTCCAGAGGACA |
| | | GAGGTTGAGGGCAGAGCTCCTGGGAGCACCAGTGGAAGTAGGAGGGCTGGGCTGGAAAACCTCCCCCAACCTCCTATTGCAAAGAGGCT |
| | | CCAGCCAGCAGCCTCCACACCCCAGTGATCTTTTAAGATGCAAATCTGCGCCATCATTTATTTCCTCAGTGCCTTCTCCAGCTCCTGGGA |
| | | TGCACACTGCCCGTCCCCAGGCCCAGAGACCTGACCACCCTCATTCCTCCCTCAGCCCACCCTGGGGTCTCTCCACCAGCTGACAGCCTT |
| | | CCTGCAGTCCCCTCCCCGAATGCTGCTCCCTGAGGCCCTCCTGGACACCTGCAGGGCAGGCACAGCCCGCGGGACCTCACAGCACTTGCT |
| | | CCGGGCAGAGCTGCAGTTTGGCCAAGTTGCCAGCTCCGTGTGGGCAGGGGCCCTGGCCTGTGGCTGCCACATCCCGGGTGGGGGCACGG |
| | | CCTTTCCTGGCGTGGATGCTGAGCAAACGTAGGGGGAAGGGGAGTGAATGAGGAGAGCCAGGTAGCTCAGGGGCTGAGGCCTCACTGAG |
| | | CAGGGTCCCGCGTGACCGGTCCCCACCGCTGACGGTTCCTGGGGTAACACTCAGGACAGGGAGAGGCAATGGAAAGAGACGTGGCCGC |
| | | CCTCGCATCCTGCAGCTCCCGCACTCCCAGCCTCCCAGCCTCCCACCCAGCCCCCCAGAGCCCACCAGTGACCCCGCCCACTGGGTCCT |
| | | CAGATGGCTCCCACGGGATCTCCTGCCTTGATCTCCTGTCCACATGGAGGTGAAGTGGGTTGCTCTGAATGAGGGGTGCCGAGCCTAGGG |
| | | CGCAGCCCACTCTCCTGGGTCCGCAGCATCACGCAGCCCGGACCACAGGCTCCTTACAAGAATCGGAAGGGTCCCTGCAATCGCCCTTCG |
| | | CACTGAGGCTTCCTACTGTGTGGTGTAAAAACACAGGCTTGTCCTCCCTTGCTGCCCACGGGGCTGGAGCCGCCTGAAAATCCCAGCCCA |
| | | CAACTTCCCCAAAGCCTGGCAGTCACTTGAATAGCCAAATGAGTCCTAGAAAGCGAGAGACGAGAGGGGAATGAGCGCCGAAAATCAAAG |
| | | CAGGTTCCCCTCCTGACAACTCCAGAGAAGGCGCATGGGCCCCGTGGCAGACCCGAACCCCCAGCCTCGCGACCGCCTGTGACCTGCGG |
| | | GTCAACCACCCGCCGCGGCTCCACGCCGTGGGCACAGACTCAGGGAGCAGGATGAGAAAGCTGAGACGGCGCAGCCACGGCCCGGTGC |
| | | CTTCACGCGCACAGCGACACAGCCCCAGCCAGCGGGGCCCACGCTAAGGCGGAATCCCACAGAAGCCTACAGAGCGAGCGCGCGCCTG |
| | | TGCTTCCCAAAACGGAATGGAACCAAGGTGACTTCTACAGAACGATCTGAAGCCCTGGCTGGCCCTTATGCTAGTCTCTTGGGAGCGTTC |
| | | CAAATGCAGCTCAATATTACTTACTTGACTTTTATCTTTCCTCCCTGGTTCGTGGTATTTATAACTGGGTCATCTTTTAACTATTTGCAA |
| | | CGTAGCTTCAGGGGAGAGGGGGAGGGCTTTATAAATAACCTGTATTATTATTATGCAGGTTGATTCTGTTCCCTGAGCTAAAGGGAACAT |
| | | GAAAATACATGTCTGTGACTCATGCCCCCCCACCCCCACTCCAGGGTGTGCTGAGGAGTCTCTCAGCTGCCCCGGGGTCCTCGAGCAGGG |
| | | GAGGGAGAAAGGCTGGCGCTGCGCCCTCCATCGCGTGAAGCCAGGGGATTTTGCTCTGCGACAAGCTGACTTGGCTCTCGTATTGTTTGC |
| | | AGAATCACCCAGTTCCAAGGCAGTCCCTGCGGGCAGGTGCAGCTGTGCGGGAGCTTCAGTCCTGTCCCCAACACCCAGGCAGTAATGGTT |
| | | CCAGCACGGAAGGTCTACCTACCTCCCACTGCACAGCCCGAGGGCTGTCCTGGAGGCACAGCCATCCGTCCCTGGGTGGGCAGGCACGTT |
| | | TATGACCCCCACCCCCACCCCCACCCCCCACGCGAGTCAGCACGTTCCATACTCGGGTGATCGTGCTCATCCCCTGGTCATGTCATCGGG |
| | | ATCTGAGTGCCATCCGAGCAGAGAGCTGTGGCCCGGTGCCGGGGGTGGACTTCATCTATTCCAGGGAACCAAGGATGCATGATTTGCAAA |
| | | CAAAACCAGAAGCGCAAGCCATCTCCTCGCCTCCCCTGATAGCCGTGCTGCGGAGCCTGAGTGCTGGAG |
| 240 | ITGB2 | CAGGAACCACGGGACCTGCTGCCTAGCGGCCCTGTTCCACCCTTGGCCGCTCGCAAAATGTTTAGGCTTCATAAGGTTTGCCCAGGGTCA |
| | | CAAATTTAACTCACAGCAAACAATGAAATCAGCGCATGATTTTCGAGCCCTCGTGGTCACCCTCCCTTCCTCCTGCCCTTTCCTGCATGG |
| | | GCAGCAGCAGGGTGAGGAGCTGCTCTCCCCAGGCCCAGGCTGGAGTCCCTCAGACGACCTGCCGGCCAGGGTACCCCCCTGCCCCCACAC |
| | | AGCGCCTGACAGAGCCCCCCACACTGGGGGAACGTGGGGACCCAAGCAGGGGCAGCGGCCTCACCGGGCAGGCGGCGACCTGCATCATG |
| | | GCGTCCAGCCCACCCTCGGGTGCATCCAGGTTTCCGGAAATCAGCTGCTTCCCGACCTCGGTCTGAAACTGGTTGGAGTTGTTGGTCAGC |
| | | TTCAGCACGTGCCTGAAGGCAAACGGGGGCTGGCACTCTTTCTCCTTGTTGGGGCATGGGTTTCGCAGCTTATCAGGGTGCGTGTTCACG |
| | | AACGGCAGCACGGTCTTGTCCACGAAGGACCCGAAGCCTGCAGGGCACATGGAGGGGCTGG |
| 241 | ITGB2 | TGCGTTTAGTGTAAAAATATCAGGTGTGGCTGCACGGAGTGAAAAATCACAGGCTCCACGGAGCCGGGAGGCCTGCTGCCCTGCCCTCTT |
| | | GCTTTGATGAGGAAATGGCGACCGCAGAAGGAAATGTAGCAGCACCGGCAACCGGCATCCGTGGGGCCACGCCGGGCTGCTTCCCAGGG |
| | | CCCTCCAGCCAAGCAGCCACAGGAAAGAGTAGATGTTGATCCCAAGCTAGGACTGAGGAGTCCGTCCCTAAGAGCCGAGGGAGTCAGGTG |
| | | GGCGAAACTGGCCGCATGTCTGGGTACAACTGCTCAGGGTTTCTCATCTGCTGAATCACCAAGCTAGGTTCTGAAGCCAGGCGTGAGTGA |
| | | GCAGGACTGGAGCAGGATTCTGGGAACAATCTTTTCCCTCC |
| 242 | POFUT2 | GCTGGGGAACTGAAGGAAGGGCTGTGGAGCCTGAAGCCTGGGCCTGGCCTGTGCTGCGGCCGCACCGCTGGGTGATGCAGGAGCCACT |
| | | CCACCTCCCTGGCACCCCAGCCTCATCCGGCAACCTGGGAGCGTGGGCCTCCTGCCCCTCCAGGGAGGCCCTGGCCGTGTCCTCATGGG |
| | | GCCCCTCCAGGTCCTTGTGGCTCCAGGTCGGGACAGTGGCTGTGAGATCTGACCCTCCCGTTCCCCCTCCACCAAGTAGGAGAAACCCCG |
| | | GAGCATGAGCCCTCGTCCTTCACCGTCCCGGGGACAGGGGGACCCCCAGATGCTGCACGGCTGACAGGCCAACGTGGCAGAAGCTCCAGC |
| | | TTCACAGGAAGCCAGTGACCATGAGAGTCTGTAGCTGTAACGAAGCCACAGAGCTGTGGCTTTCTTTCCCCTTCAGCTCTAGGAAAGGTT |
| | | ATCTGCCCTGCACAGATCTCCGGAGGCCTGGCTGGGCTCTGAGAGCATCAGACTGATTATCGTAAGAAAATAATCTCTGCAGACACATTC |
| | | CTTGCTAGAAGCAGGGGACAAAGCCCAGCTTCAAAGACAATTCCACACACGCCCTCCCTGCCCTGCACAGCTGCCTGCCGGGTGGGAGCA |
| | | GAGCCCTTGCAGCCGGGCTCAGGGGCCTGGGCAGGGACAGCGTGTGGCAGGGGCACAGCTGAGACAGGAGCCTCAAAGCGACACCAACC |
| | | CGACGTGAAGCTACAGTTGAGGAGACACAGCTGCCCCCATTCCCGGGCCTCATCTCCACAGTGAGACGCTGGACTCTCTCCCTGACCCAC |
| | | CGTCTCTTAGAACCTCCCCTCCATCCGGAGCAGTTCGGCAGCCCCAGGGCAGCCAGGGGAACCCTGCCGAGTGCCTCTGGGCCGCCACA |
| | | GACCGCAGAGCCCGCGGGAGCCTTGCTCACACAGCCTCAGGTCCACTGTGGTCTTGGGGGAAAGCCCTGTCCTGGGACAGGGGAGCCG |
| | | GGGGTCCTGGCCCTGGACCACCATCTGGGGACCACGTTGTCACGCCTGCAAAGCTCCCTGCCCCACCCCCATGTGCCGGCTGGTGTTGA |
| | | CACCTTTGTAGAGTGGGAACCTGCCTCCGACCCCAGCCTGCAGCCACAGGGCAGGTTATAGACCAGGTGAGAGGGCGCCGCGCCCAGAA |
| | | CCAAGGAGCACAAGTCCGCAGTGCCCATGAGATCCTCATGCTGGCCGGCGCAGGAGCCATCCTCGGCCTCTGCAGGTCCTCGTGGGAAA |
| | | CCGCGGGGGCACGTGGGGCGGCTGCAGGGTCCGCAAAGCCGGCTGTTTGCGAAGGGCGCAGCTCCACCTGGAACAGCCGAGGCCGCC |
| | | CACGCGCTTCCCGCGGGATCAGAGCAGCCTCCACGGCTGTTGTCTCAGGCACCACGGGATGCCTTTCTTCGTTTCAATAGCTGTGGGAAA |
| | | GCCTCAATCGGTCCTGAAAGAACCCAGATGTGCAGCAATGACAAGGCCTTCTCTGAGACTCTAGAACCTTCTGCCATCTCAGACAGGAGG |
| | | GAGCCGTGAGGCAGGCGGGAGATTTGCAGTCAGCAAAGGACGGGCAGGTGGGGCAGCTGCACACCCAGGGCCCTCTCCACGGTCTTCCC |
| | | GGGCCCACCCCTCCCGCGGTCCTGGGTCATCCACCTGCTGGCCTCACTCTGCCCACGCGGCCAGGTCCCACCGGCCCCTGAGCTCAACA |
| | | GACCAAAGCTGGCCCGACCCCACCCCCAAGAAGAATGAAACAATTTTTTTTTACCTCTTGCAGAAAAGTAAAAGATCATTTATTCATTCT |
| | | GTTTCTAGATAGCAAAACTAAGTGTCAAAAGCACCTTCTGCACACAGTCTGCACACACTGGCCGGTGGTCCTGTTCCCGCAAGGTTGAGC |
| | | TGTGTTCCAGAGACATGGGTCCTCCGGGTGATGAGGAGCCGCTGGAGGGCCCTGAGCTGCACGTGCTAATGATTAACGCCCCGTCCGTGC |
| | | TGGCCGGTTTCTCAAATGCCTCCTGACGATTGCGC |
| 243 | chr21: | GGCCTGAGGAGTCAAACGGTGCAAACCCTGCCCCACTCTGTTTGGGAAGCACCTGCTGTGTGGCAGGCGCTGCGCTTGGTGCTGGGGAT |
| | 45571500- | AGACCATGGGGAAGAAACACACAGAACCTGCCCTGCTCTCAAGGAACAGGCCCTGGGGGCGGCCAGGGGCAGAGACCCAAGGCAGACAC |
| | 45573700 | CCACACAGTGGCGTAATGACAGTGCTTATGGTGGGGACCTGGCTGCACAGCAGGTCAGCAAGGGGATGTTCAGGTGACACTGGGGGCAC |
| | | GGAGACCCAGGGGAGAGTGGATTGACAGAGGGGACGCTGGGCAAATGTCCCGAGGCTGAGGTGGAGTTGCGGGAAGGAGGAGGCTGCC |
| | | GGGCAGAGGCGCAGAGAGCTTTGCAGGTGTTGGCAGAGACCAGCAGGCCCTGCGAGGCCTGGGGTGTGTCCTCAGCTGGGAGGGCCAT |
| | | AGAAGGATCTGGGCTTGCAGATGCTGGTGCAGACTGGAGGCCTGGGGTGTGAGAGTCCAGGCGGGGCTCCTGCCAACACCCAGGGGAGT |
| | | GGGCCTGGGCCAGGTGGACCGGGAGCTGGCACGGTGGTCAGGTGCTTGGAGGCTGCGTGCCACGCTGGGGACCTGGAGGTGTGTGAG |
| | | GAGGTGTCTGTTGCTCCTGGGGCTGCCGCCTGCAGGGCTGGGTGTGCAGCAGTGCGGGGCAATGAAGTGGGCGGGTTCTGGGATGGTG |
| | | GACGTTCCCTTTGTTGGGAACGTGTTGGTGCCAAGCTGCCATTTGAGTTTGGCTCTGAGGGGTCTGGGCAGGGGACACACAGGGAATCAC |
| | | ACAGGATGGAGTGAGTTCCCAGGGACCCAGGGTGGCTTGGCCTGAGAACAGCTCCCACTCCCAGATGTGTGGGAAGCCCTCGGCACCAA |
| | | GCCTCAGCCTCTCCATCTGTGAAATGGAGACAACGTCACTGGACTTGCAGGCTGTCCATGAGGGTGATGCGATCAGAAAGGGTGGAGTTC |
| | | CTGAACGCCCCGGGGTCGGGGTCTCACAGCAGGAGCTTAGCTGGTGTCGGCATCTCCTGGACCCGTCCTCAGCTCCGAGCGCCCAGTCC |
| | | TGCCACCTGTGTCCAAGTCTGCACTGTGCCCACGAGGCCCTCAAGGCCGCAGACAGCCCCACACTTCTCGGACGCCGCCCCAGCACGGT |
| | | CCTTGTGTGAGGTGGACACTCCTTCTGGACGCCGCCCCAGCACGGTCCTTGTGTGAGGTGGACACTCCTTCTGGACGCCGCCCCAGTACG |
| | | GTCCTTGTGTGAGGTGGACACTCCTTCTAGGGAAGGAGTAGTAACTCTTGGGTGGTCGGGTAGTTGCCATGGAAAGGGGCAGTAATGCCC |
| | | AGGTATTGCCGTGGCAACCGTAAACTGACATGGCGCACTGGAGGGCGTGCCTCATGGAAAGCTACCTGTGCCCCTGCCCTGTGTTAGCTA |
| | | GGCCTCAATGTGGTCCAGTATCTGAGCACCGCCTCCTGCCTCAGATGTTCCCGTCTGTCACCCCATTACCAGGGCGGCACTTCGGGTCCT |
| | | TTCCAGCCATCATTGTCCTGGCATTGCCACAGTGGACACTGCCACACAGGCTTGTGTGCTTGCGCGTACCCAGGTCCTCACCTCTCTGGG |
| | | ATAAACCAGGCACGTGGCGGCCGCCCCATTTTCCACCCGCCAGCGGTGGAGGAGTTGCCCAGCCTTGCAGGAAAACAGCTCTCATGCCAG |
| | | CAGCGGAGCATCCTATTCAAGTTTTCTCAGGGCTGCCAGCACAAATGCTGCATGCCGGGCGGCTTCCTCAGCAGACCGTTGTTTCTCTGC |
| | | GTCCTGGAGGCTGGACGTCCCAGGTCCCCGTGTGGCAGGCCCGGTTCCTCCCGCAGCCTCTCCTTGGCTTGTGGGCGGCGTCTCCTCCCT |
| | | GGGTCCTCGCAGGGCCACCCCTCCGTGTGTCTGTGTCCTCCCTCCCCTTATAAGGACCCCAGGCAGACTGGATCAGGGCCTGCCCTAAGG |
| | | ACTGAATTTTACCTTAATCACCTCTTTAAAAGCTGTCTCCAAATACAGTCACCTTCTGGGGTCCTGGCTGTTAGGGCTTTGATGCATGGA |
| | | TTTGGGGGACACCGCTCAGCCCCTAACAGCCCCCATCCTCTGCCTGCCTTTACCATGGGGCTGAGCCCAGCCCTGCAGGAGTCCCCTGGT |
| | | TTGATGTCTGCTGTGGCCACGGCGACCCTCAGGCTGCTCCAGCCGCACTTGTGCTT |
| 244 | chr21: | GGGGAGTCTCCAGGGGCTGGGGCTGGAGCCGCATCAGAGAGGAAAGGGGTGTTTGAAAAAGGGGCAGGGCCTGGGACCCAGGAAACTG |
| | 45609000- | TTCTTCCAGAGACACCCGTGAAGCTGAGCTTTGCCTCTCAGGGAAGCTGTGACCCCACGGGTGCTGCCCAGAGAGATCGGGCCAGGTGGA |
| | 45610600 | GCCAAGATGGACTGGAATTCCCCGACGGGGACAAGGGGCCGGACGAGGCTGACTTGCCCTGTCTGATGAATGGTCAGGTTTGCTTTTTCT |
| | | CCTGAAAACACGAGGCAGTGATCCCGGCCAGCTAATTCCAGCAGACTGGAGACGGGATGGTGGAGAATGAGGCTGTGGGCGGGAAGAGC |
| | | AGATGGGACTCGCCAGCATCCTCACGGCAGGGCCGCGCTATTGCCCTCCCTCCCCTCCTACTCTCTGGGGTCCCAGGAGCCCCAGATACG |
| | | CAATGCTGCCAGGCGATTTCTGGCGCCCCGCAGACCCCTGCCCCTGGAGTTGGGCCAGGTCCCGGCTGGAGCAAAGGGGGCTCCTTCAA |
| | | GCCCGCTCCTCCCTGTCAAACCCGAGGAGCCTGACAGGCGCAGCGTCACCAGCGTCACCGGGCCATAGTGAGCGGCCAAGCCAGCGTCA |
| | | CCGGGCCATAGTGAGCGGCCAAGCCAGCGTCACCGGGCCATAGTGAGCCGCCAAGCCAGCGTCACCGGGCCATAGTGAGCCGCCAAGC |
| | | CAGTGTCACCGGGCCATAGTGAGCGGCCAAGCCTTGGTCTGCCAGAGCCGGCCGCACCAGAAGGATTTCTGGGTCCCCAGTCCTGGAGG |
| | | AGCACACGGTTTACACCAGGCCTTGGGAGGGGAAGAGGCAAGGCGTGGGCCCAGCCCTCACTCCCCAGGAGAAACCCTGTTTGAGCGGC |
| | | AGAGGAGACTGGAGAGACCCCAGGGCGGGGATCCCTGAGAGGAGAGAAACCCGGAATTCATCCACGGAGGCGTTCACCCAGAGGAGACC |
| | | CGGAGCTTCTCCAGGAGAGGCTGGATTGCTCCAACAGGGGCCCTGAGGAGCTGATGGCAAGAGCGGAAGGCAGCTCTGACTCGTGCGTC |
| | | TGACTCCAGGTGTGGCCGTTGGGGCTACAGTGGGACCAGCCTGTTGTCACTGAACCCACAAAGTGCCTCCGAGCGCGGGTGGAGAGAGG |
| | | GGGACCTCCCACCGTCTGCTGGCCTTGAATCTTGAATCTAATTCCCGTCTGTGCTTTGATGGGAGAGGCACTGGGAGCGGGCGGCTTTTT |
| | | CAGTTCCTTTTATCTTGAATGGCCTTTGGGGGATTTTCACAGATTCTGAGTTCAAAGCCCAGGGAGGTGTGGGAACGTGACATTCCTCAC |
| | | CGCATTCCTCACCGCATTCCTCTGTAAACCAGGCGGTGTTGGCACCCATGAGCCTGTGTCTTCTATGACATCAGGAGTTTTATCCCTCAC |
| | | GTCAGAAATCAGGGTTCCAGGCGCCTTGGTTTTTCTTGGCGCCAGCGGCTTGGCTATAGAAGAAAAACTGAAGGGGCCAGGTGCGGTGGC |
| | | TCACACCTGTAATCCCAGCACTTTGGAAGGCCAAGGCGGGTGGATCACGAGGTCAGGGGTTCGAGACCAGCCAACATGGCAA |
| 245 | COL18A1 | GCTCCTCAGGGGGAGGTTCGGGGCCTTTGGTCTCTGGACTTGGGCAGCAGAAAGGAAACATCCCTGGGGGCCTGTGGTGACCCCCATCC |
| | | TCCCCAGGGTGGTCTGGCAGGGGACACTGTTTTCCAAAGCAAAGCCAGAGCGCCAAGGGCTCTCGGGATTCACGAGATCCACATTTATCC |
| | | CAAGTTAGAACAGCACATCTGTGCGTGCAAACTTCATTCTGACTTCGGCCGGCTGTCCTTCTTGCCCAAAGCACCGTGAGGCCTCATCCC |
| | | TGCATCCCTGTTGCTTCTTTCATGTGGGATGAGAACCCAGGAAGGGGCTGAGTGTGACTCCTCTGGTTTTTAGAGAGCACTGCCCCCGCC |
| | | CCGCCCCCTCCTGCTTCCCCACCTTTTCACAGTTGCCTGGCTGGGGCGTAAGTGAATTGACAGCATTTAGTTTGAGTGACTTTCGAGTTA |
| | | CTTTTTTTCTTTTTTTGAGACAGAGTCTCGCTCTGTCGCCCAGGGTGGACTGCAGTGGTGTAATCTTGGCTCACTGCAACCTCTACCTCC |
| | | CGGGTTCAAGCGATTCTCACATCTCAGCCTCTGGAGTAGCTGGAATTACAGGCGCCCGCCACCACACCTGGCTAATTTTTGTGTTTTTAG |
| | | TAGAGATGGGGTTTCACCATGTTGGCCAGGCTGGTCTCGAACTCCTGACCTCAGGTGATCCGCCTGCCTTGGCCTCCCAAAGTGCTGGGA |
| | | TTACAGGTGTGAGCCACCGAGCCTGGCCTGGAGTTATTTTGGGAGAGGGCAGCCCCTGGTTCAGCGTGGCGAGGCTGCGCTTGCTCTCCC |
| | | GGGCGGGCGTCCACACCCTCCTCGCCGAGATGGAGAAGCCCAAACCCCTGCAGCGCTCCCCCATCACGTCCGGCCCTGGAAGCCCCCGGA |
| | | AACCCTGCCACGCCCTGAGTGGGAGAGCGCAGGTCCCTTTCCGGCCCTGGAAGCCCCCAGAAACCCTTGGGTGCCAGGCCTGGCCGGGAC |
| | | AGCAGCGACACTGCATGCTCAGCCCTTGCGTGAGACCACGGGAGTGTCCGCCCTCTGCACGTGCTGCTGATTGCCCACTTCGTCCAGCAG |
| | | GTTTGGGAGCTTGTGGCTGCATCCTCCTGCAGACACTTGCCCATTCTGGGGCCTCCTCTCTGTCTTTTCTCCTCTGTTGAGGGGTCTGGG |
| | | AGGGAGGCCTTGGAGGGTACCCATGCTGCTGGGACTGATGCTCCCCGCGGTGGAAGGAGCTGCCTCTTGAACAGCAGGGGGCTGAGCAGA |
| | | GGGGAGGGGATGCGGGGGTGCCGTGCACACAGGTGCTCTCAGGACGCAGGGGCTTCTCAGCCCTGCTGTCCCAGGGCTGCACTCCAGCAG |
| | | GGCAGACTCCTGAGGTGCAGACACCCCAGCTTCACGCTCACACTTCTGGAAGGCGATGTCTGTGCGTTTGCTTTCTGCTGCAGTTTAAAA |
| | | AGCCGGGCTCTCTCCGGAGCGTGTGTAGGGCCTGGTCACTGGAATATCTGGACTCAGTGTTAATGGCAGCCACGCTGGGGGCTGGGCCCA |
| | | GCTTTCTGTTCTCCGTGTGGGTGCCATATCCACCTCCATCGCAGCCCTTTCTCTCTCGACCTTTTAAATCACAGTGTCACCTCCCCCTGC |
| | | TGTCCTGCCAGTGGCCCCTGGAGGCTTCTCCCCACCCCTTTCTTCTGGGGCAATTCTTAAGGCTGGCATTGAATCAGGAGGCCAGATGTG |
| | | GCCCCTAGTAACTCACCAGCAGTCCCTGAGGCTTCTGGCTCCCCTGGCCCACCAGCCTCCCATGTCTGCCTCAGGCCTCTTGACCCGCCT |
| | | GGCACTGACCAGACTGTGTGCCCGGGTGCCGTGCCCATGGGCTCCGCCTCCCCCAGGCAGGCCCCCTCTTGCTCCGCGGCCACCCCTGCT |
| | | CTTGACCTCACACCTCTGCGGTGTGTCTGGACACACCAGCACCACGGCGGGCGGGGAGCGGAATTCTCCAGGTGGGGTGGGCAGGCCGGC |
| | | GGGTGTTGAGGTCTCTGTGCATGCTTGTGCGTACCCTGGACTTTGCCGTGAGGGGTGGCCAGTGCTCTGGGTGCCTTTGCCAGACAACTG |
| | | GTCTGCCGGGCCGAGCATTCATGCTGGTCGCCATCACGTGACTCCCATGCGCCCTGGCCCTGGGGTTGGGTCTGCAGGACTGAGAACCA |
| | | GCGGAAGGGGGGCGAGGCCTCGGGAATGCGCCGGCAACTGGCGATGAGCTCAGGCCTGACTAATGAGCCCAGGTGACTCATACACCCG |
| | | GGGCCTGGATGAGTCTGACTGGGTCAGGACTTCCCTGCTTGTTCTGTCCTGGGAGATGTTGTCCCTGGCCCTGCAGAGCCGGGAGGACAC |
| | | GAGGCCTCCTGGGTCACAGCCAACGCAGCCTACTCCTGCCCACTGCTCGCGCCGGCCAAGGCCCGTCGGCACCACCTCCTCCATGAAGC |
| | | CTTCCTGACTGCCCCCATCCCTCTGTGGGCAGCTCGAGTGTGCATCTTGAGTGCTGTGCAGGTTGGGGTCCGGCGCTCCTGCAGGCAGGC |
| | | GGCGTCTGGGCCTGGGGGCTCTCAGAGTTTGAGGAGCGTGTGGTGAGGGTGGCCTCGGGCCTCAAAGACGCAGCGCTGTGGGAACCGG |
| | | GAGACTGGCTGAGCCCGCTCTGAGGAAGGTGGGGCCAGGGGCACCCTCAGCTGACCCGGCGTGCAGGGGTGACCAGCCAGGCGTGGCC |
| | | AAGGATGGGGTCTCTGGGATCAGGAGACTTCAGTAGCAGCCAGGACCGAGGCCACCAGTTTCCACCCTGGCATTTTCCATCTTTTGAAGG |
| | | ACTGGAAACGATTGGATTCTTTAACTTTTTTAAGTTGAGGTGAAATTCACAACGCATAAAATTAACCATCTTAAAGCGAACAATTCGGTG |
| | | ACATTTAGTACAGCCAGAAGGCTGTGCAGCCATCACCACTGCCCAACTCTAGAACATTCACACGCCGGAGAGAGGGAGCCCTGGGCCATC |
| | | ACGCAGCCACCGCCCGGCCCCAAGAACCTGCGAGTCCACTTTCCACCTCTGGATCGGCGGTTCTGGACGTTCATGCAGGTGGTTCCCGCA |
| | | GTGCGAGGCCTTTTGTTTCGGGCTCCTCTCACAAGCCTCACGTTTCCAGGTACGTCGTGGTGTTGTGCAGACCCACAATTCATCCCTTTT |
| | | CATGGGTGTGTAATAGTCCACCATAGATTCTCTACGTTTTAAAGCATGTTTTATGTGCCTGAAATGTCTCTGCACTCGAGACTATAGCTT |
| | | GCTTTCTTTCTTTTCTTTTTTTTTTTTTAATTTGAGACGGAGTCTTGCTCTGTTTTCAGGCTGGAGTGCAGTGGTGCGATCTCGGCTCAC |
| | | TATAACCTCTGCCTCCCAGGTTCAACTGATTCTTTTGCCTCAGCCTCCCGAGTAGCTGGGACTATAGGCGCGCCACCCCACCCGGCCAAT |
| | | TTTTTTGTATTTTTAGTAGAGATGGGGTTTCATCATGTTGGCCAGGATGGTCTCGATCTTCCGACCTTGTGATCTGCCCGCCTCGGCCTC |
| | | CCAAATTGTTGGGATTACAGGCGTGAGCCACCGCGCCCAGCCGAGACTACAGCTTTCTTTAACTGCATCCCTGGAGGGATCTGAGAGTCT |
| | | CTTTCCCTGTCTCCTTTCCTTTGGAAAACATTTCAGCCAGGGCTCCCCAAGATGAAAGGCCAGAGTCCCAGGCATGGGCGTTGCAGGTGC |
| | | ACAGTTGCCACGGGGAGCTGTGGGTGATGGTCGCTGTCAGCGATGGCTGCTGCAGGTCCCTGTGAGGAAGGGGCAGTGCCACAGCAGGAG |
| | | GAGAGGGAGTCAGCGGACGTTGATTGGCAGTGCCCGCCCATTCCATCATTCAGTCACCCACTGTGCACCCAGCACCCAGGCTCGGCTGCA |
| | | TAGAACATGGCCCAGGAAGGCTCCACTTCCTGTCTCCTCTTCTCCCCTCTCCAGTCTCATGATGGGGCTGGAGGCATCTTCTAGTTTTGA |
| | | GTTCTGAGCTAATGAACATGCTCATGAGCAGGCGGCAGGATCCCAGGACGGTGGAGCTGGGAGCCTGACTGCGGGTGACGGACAGGCTCT |
| | | GGCAGCCCCTGTCAGCATCCTCTCCAGGGCATGTGAAAGCCAGTGTGTCCTCAGCTGCCAGTGCCCCCTCCCCACCTCCTCTGGGCCCAT |
| | | GTGCACGGGACCTGGGCTCCCCCAACCAAGCCTGCCCGCCTTGGTTCAGCAGAACGGCTCCTGTCTCTACAGCGGTGCCAGGCCAGGAGT |
| | | GCTGTGTCTGTGAAGCGGGGTCATGGTTTTGGGGCCCTCATCTCCCTCGCGCCCTCTCATTGGGGACCCCCCGTCTCCCTAGCGCCCTCT |
| | | CGTCCTCTCCTGCATGTGCTGTGTCTGTGAAGCGGGGTCATGGTTTTGGGGCCCCCCGTCTCCCTAGCGTTCTCTCGCCCTCTCCAGCAT |
| | | GTGAAGTGGGGTCATGGTTTGGGGGCCCCCATCTCCCTAGCGCCCTCTCGTTGGGGACCCCCCGTCTCCCTAGCGCCCTCTCGCCCTCGC |
| | | CTGCATGTGCTGTGTCCATGAAGTGGGGTCATGGTTTGGGGGCCCCCTATCTTTCTAGCACCCTCTCGCCCTCTCCTGTATGTGAAGTGG |
| | | GGTCATGGTTTGGGGGCCGCCATCTTTCTAGCGCCCTCTCGCCTTCTCCTGAGCGTGTGGAACTCTGTGGTGGTCAGAGCTAAGGTTCTG |
| | | AATAGGTCGAAGCACCTCCCCGGTGCCTCTCACCCTGAATGCTCTGGGAGGACACAGCCTTTTCATAGGCTACGACTGACATGGCAGGAG |
| | | GGGCCTGCCTGCCACCCGGGTCCTCTGCTGCCTGCTGCTTGCTGGGGAGGGGGCTCGAGACTGGGATCCTGGGCTTCTGCTCCAGCTGTG |
| | | CCCAAGGGAGCTGCTGAGGAGGGACCGGGTGGGGCATCCACTCTGGGCAGGTTCAGGGTCATTCTTGGTGACCCCGGGTCCGGTTACAAA |
| | | GGCTGATGGAGCGCGTGGGTGGCTGCCTAAGTCTCTGGAAGCCCAAGAATGTGGAGATGGCGCGTCTCGGCCCGGGGTCTCGTGGCTGGT |
| | | CTGGGAGAACTTGCCTTTATTTCTAGGCAGGAGGCTGCACTGCAAGGGAGCGTCAGTGGCCCGGCTGGCTTTCCCCGGCCCTCAGCCCGC |
| | | ACTCGTCCACCAAAGCAAGCTCCTTTGTGGGGCTGCCCTGGGAAGCCGGGATCACGAGGCTCTGCCGGCCGTGGTCACCCCATGAGGCAG |
| | | GGTCAGCTCGGGAGCAAGGCGGATCAGATGGAACAGAACACGTAGACCACCTCGCCCGCCCTTAGTCAGCTGGGCCATTGAAAATCAAGT |
| | | CCGTAGAAAGACCTAGAAATAAGTCCCGGGGTGCCCTTGCCTGTTGACGGGCGGGCCGAGCAGGACTGTTCTCAGGCAGGCACTGGTCTC |
| | | TTGGCTTCCAGGTGGTTTGTTTGCTGGTTTGAGGCTGGGGGTGACGCTCCTGTGCGGGAGGAGGTCGCATTCCATTCATAGCGGCTTATC |
| | | TGGGCTGTCAGGCAGGCCTGGGAGGGAGCCTGCCTCTGTGCTCTCCAAGGGTGGGCGACGGACAGACAGGGTGTCCCACCCCTTCTGGGC |
| | | CAAGGACAGAGGGTCAGTGTTTGCAGAGACCTGGGGAGGCCCAGGTGACCTCCACCGAGCACCTGCTGTGTGCAGGGCCAGTGCTGGCTG |
| | | CAGAGACAGCGGAGCGTGTGTGGACCCGGCGGCCCAGGGGAGGGGGGCAGGCAGGACCCGGCGGCCCAGGGGAGGGGGGCAGGCAGGACC |
| | | CGGCGGCCCAGGGGAGGTGGGCAGGCAGGACCCGGCGGCCCAGGGGAGGGGGGCAGGCAGGACCCGGCGGCCCAGGGGAGGGGGCAGGCA |
| | | GGACCCGGCGGCCCAGGGGAGGGGGGCAGGCAGGACTCGGCGGCCCAGGGGAGGGGGGCAGGCAGGACCAGGCGGCCCTGGGGGTC |
| | | AGGGGTGGAGGCCAGGCCTAGACGGCCCACAGGAGGGTGGACTCATTCTGACCGATTCCTGGAAGCCCCCGGAAAGTGGTGATGTTCTG |
| | | GAGGGCCCAGCAGACCCCAAGGCCCCCAAGACAATCCCAGCTGGCTCTCTGCGGCTCTCGGTGTCTGCCATTTGAGACAATTTGGGCACA |
| | | GGCAGGGCAGGCCGTCGCGGACGGTCTAAGCCGCGCGCATTGGTGGGGGCAGCAGAGCCCCTGCTCTCAGCTCCTCGGGGTACAGCGG |
| | | GGGTACCAGGCGGGTGAGTGGGTGGGTGGTCACTGCTCCTGCCAAGGGCAGCCCTGGTTTGGTTTGCACTTGCTGCCCTGGTGACGGCT |
| | | GCTCTCATTCCTGCCCCATTGCTAACAAGGGTGTCATAAGCTACTTTCCCGGCCCACATCCTATTAAGCCCATGGAGACCCTCCCACAGC |
| | | TGAGCCTGCTGTGGGCTGCAGGCCCTGGGCGGTGCCCACCTCGGTCCCCACTGGCCTCCTTCCAGCACTTTAGAGCAGACACAGGTTGGA |
| | | GATAAGGAAAGTTCCAGAGCACAGACTGGAACAAGCCCCAGGCCTCTCCCTGCCCCAGCAGGGCCTCCCTGGATTTGGGGGACAGGTGC |
| | | CCTCATGGGGGGTCCTGAAGGTCAGAGCTGGGGCTGGGGCTGGGCTGGCGGAGGTGGCCTTGGCGGAGGCCACATTCCAGGGTCTCAG |
| | | TGAGAGTCTGTGGCAGGCAGCCTTGCAGATGCCGCTGAGGGACCCCCCACTTCATGTTGTGGGTGATGTGGTCCATTGATTGCCTCCAGG |
| | | TTTAAATCAGGTGGATATTTACCTAGCGGCCTCCTCTCCCTCTGCACAGGGCCTGGAGTGGGATGGACTGGGGTGCTCAGCTGGAGGCTC |
| | | TGCAGACACAGCCCCCTGGGCTATGCAGGCCCTGCTGGGAGCCACATTGCCATTTTTCATCACCCACTTTTTGGGTGAGAACCCCCTCGA |
| | | GTCCTAACATCTGCCGCATCTCAGAGCCTGTGGCTCCAGTCAGAGCATCTGGACCATACTGCTGGGGTCAGAGCGCGGCAGGACAATGGC |
| 246 | COL18A1 | TGCCACCACCATCTTCAGGTAGAGCTTCTCTCTCCTCCTTGCTGGGCGGGGCCCCTCCCTGGGGAAGCCTGCAGGACCCAGACAGCCAAG |
| | | GACTCTCGCCCGCCGCAGCCGCTCCCAGCCAGCAGCTCCAACGCCCTGACGTCCGCCTGCGCACGCCACTTCTGCACCCCCTGGTGATG |
| | | GGCTCCCTGGGCAAGCACGCGGCCCCCTCCGCCTTCTCCTCTGGGCTCCCGGGCGCACTGTCTCAGGTCGCAGTCACCACTTTAACCAG |
| | | GGACAGCGGTGCTTGGGTCTCCCACGTGGCTAACTCTGTGGGGCCGGGTCTTGCTAATAACTCTGCCCTGCTCGGGGCTGACCCCGAGG |
| | | CCCCCGCCGGTCGCTGCCTGCCCCTGCCACCCTCCCTGCCAGTCTGCGGCCACCTGGGCATCTCACGCTTCTGGCTGCCCAACCACCTC |
| | | CACCACGAGAGCGGCGAGCAGGTGCGGGCCGGGGCACGGGCGTGGGGGGGCCTGCTGCAGACGCACTGCCACCCCTTCCTCGCCTGG |
| | | TTCTTCTGCCTGCTGCTGGTCCCCCCATGCGGCAGCGTCCCGCCGCCCGCCCCGCCACCCTGCTGCCAGTTCTGCGAGGCCCTGCAGGA |
| | | TGCGTGTTGGAGCCGCCTGGGCGGGGGCCGGCTGCCCGTCGCCTGTGCCTCGCTCCCGACCCAGGAGGATGGGTACTGTGTGCTCATTG |
| | | GGCCGGCTGCAGGTAACTGGCCGGCCCCGATCTCCCCACCCTTTCCTTTTTGCCTTGCCAGGTAAGTGTGGGCGGGGCTGACGTGAGCCT |
| | | GGTACAGGTTCCCCCCACATCGAATCTCTACGTTCAGGGGCCCGTGGCCCTCGGGAGGTGGGAGAGCTGGGAGTGAGGCCTCCTGTGTG |
| | | GGGAGGAGGCCGGCGTCTGGACAGGAAGAGGGCTGGATGAACCGCAGCCGATGTGTCCAGGTGCCACCTGGGCCTGGAGCTCCCTGAG |
| | | CATTTTAGCGCATTTAGTCCTCAGCACGGTCCCGAGATACCCTGCCATGCCCCGAGTCACAGAGGGGAAACTGAGGCGTGGGGCAGTGGC |
| | | GTGACTCACCCCAGGGAGCCGAGATTCCCGCTCAGGTGTGGCTGCATCGACCTTGCTCCGGTCACTAAGCTGCACGGTTCGATGCGCTTC |
| | | CTGGGAGCCCCAGCGTGCTCGGGCCAAGGGTGCTGCCGCGTGGGCAGTGCAGAGACCCTACCAGCGTGGGGACCAGGGAGGTCTGCAG |
| | | GGCCCGTCCTGAGAGGGAGCCTTTCATGTCCCCCTCCCCATCCTGAAGCACACAGCCTCCCTGCCACAGTGGGGGCCGCTTCTGGGCCC |
| | | AGGGGACGTTGCCCCATCACCGTGTGGCCTGGCCTTGTTGCTGGCTGGACAGTTGGGGGCAGGAAGAGGAGGGAAAGGGGGACTCTTTA |
| | | ACCTCCTGGGGGCAGGGGCAGCCCAGAAAGGACCCCAGCAGATCCCTCCTCTGTGTCCGGGAGTAGACGGGGCCCC |
| 247 | COL18A1 | GGGCTCCACAGCGGCCTGTCTCCTCACAGGGTTCAGCCCAGTCTGCTCTCACTCATTTGCTGATTCATTCTTTCATTCAGCCAGTCAATA |
| | | GTCATGGCCCCTCCTGTGTGCCGGGTGGCCATGGATATTGCCCTGGGTAACACACAGCCTGGCCCTGTGGAGCAGACAGTGGGGACAGCC |
| | | ATGTGGACAGGGTGCAGGTGGATGGCAATGGCAGCTGGGTCAGGAGGGGCTGAGGGCCGTGGGGAAAGGTGCAGAATCAATAGGGGCAT |
| | | CCGGACTGGGGTGCAGGCCTGGGGGCTGGGATTTCTAGGGTGGAGGTCACCTCTGAGGGAGACAGAGCAAGGCCCTGGGAGATTAGAA |
| | | GGTCGAAGGTCGCCGTGTTGAGGTCAGGGGCCCTGAATTGGAGCCGCGGCAAAGGAGAGGGCAGGTCAGGGCACGTGGTGAGTGATTG |
| | | CTGCGGCTTCTGAGCACGGCTGGGTCTGTGGGGCCTGAGCAGAGGTGACCCGCGATCCGGCGCCACGGCAGGCAGGACTCCCCACCCT |
| | | TGCTGCTGCCTACACCCCCAGGGCAGCCCCAGAGTCGGGGGCGCAGCTCCCTGCTTGCCAGTTCAGAGCCCAGCCCCTCTCACCCAGCC |
| | | CAGAGGAGGACACAGATGGAGGAGGGGCACCCGGAGGGTCCCCCCGCCGACAGGCCCCACGTCTCCCACCTGCAGGACAATGAAGTGG |
| | | CCGCCTTGCAGCCCCCCGTGGTGCAGCTGCACGACAGCAACCCCTACCCGCGGCGGGAGCACCCCCACCCCACCGCGCGGCCCTGGCG |
| | | GGCAGATGACATCCTGGCCAGCCCCCCTCGCCTGCCCGAGCCCCAGCCCTACCCCGGAGCCCCGCACCACAGCTCCTACGTGCACCTGC |
| | | GGCCGGCGCGACCCACAAGCCCACCCGCCCACAGCCACCGCGACTTCCAGCCGGTGGTGAGTGCCCCCCCAAAGTGGGCTTGGCTCCAT |
| | | CTAGCCCCTCGGCTCTCGGCAGCAGAAGAGGGCCCAGCCCCTGCAGAGCTGCTGGGGGTCCCAGGCTTCGGCCATGGGTGGGGGTCTG |
| | | GCGGCTCAGGGCCACTCAGGGCGGCTTGGCTGGCCCTGGGACTTGCCCTCTGGTGGCCAAGCAGTGGTCATGAAAGTCCAGCCGCTGTC |
| | | ACATCCTTGAGGAACCGGCGTACCTCCGCCTACAGCGGCAGCTGGGGGCACCCACGTGGCCCGGGGCTGCTCTGACCTGGCAGCGTATG |
| | | GGGGCTGCTGCCTGGGCCCCTCAGTGTGTCACTTGCGCGCCTCCCGCTCAGCGCCCCTCGGCCGTGCCTGTCCACACAGGTGCGGGGC |
| | | CGGGGTGGTGCGCCCGGGGCCTGGGTGCAGGGGGCAGCGTGGGACACAGCCCGTGACGCGCCCCTCTCCCCGCAGCTCCACCTGGTT |
| | | GCGCTCAACAGCCCCCTGTCAGGCGGCATGCGGGGCATCCGCGGGGCCGACTTCCAGTGCTTCCAGCAGGCGCGGGCCGTGGGGCTGG |
| | | CGGGCACCTTCCGCGCCTTCCTGTCCTCGCGCCTGCAGGACCTGTACAGCATCGTGCGCCGTGCCGACCGCGCAGCCGTGCCCATCGTC |
| | | AACCTCAAGGTGGGTCAGTCCAGTCCTGAGGGCGCGGGCTCCTCGGCCCCCACTTGACCTCTGGGGTGAACTCCCAGCGGGGAGCTCCC |
| | | CTCTAGGGCCTCTGGAGGCCACCATGTTACAGACACTGGCGCCTAGGCTGGCGACTTCAGGGCAGGCTCCGGGTGGGTCACACCCCTCC |
| | | AGGCTCAGGCCAGGCCTCTGCATCCCTGGGCACTGCCACGTCCCCCAGGGCATCCCATGAGGCCCCCCCGTGGCCCCCTGACCCCCCGC |
| | | TCCCCCGGCAGTGCCCCTCAGAGGGTCCCATGCTGCTGGACCAAGTGTCCACACAGGTGATAGGGCTCACATACAAGCCTGGAATCAGGA |
| | | ACCGTCCTTTGGGCCTCTAGTGCCATGCGGGCTGGTGGCCCCTCTGCCA |
| 248 | chr21: | GCCTGGAGTGTAGTCCTGCTGAAGGCCAGAGACCACACACTCCACCCAGACTCCGGATCTCCCTCCCCAGCAGGGGGATGGAGGCCCTGC |
| | 45885000- | CGCTGGGAGTGCTGGTGTTATGTGGAAGGGCTGGGCTTCTCCAGGGCTCCTGGGAGGCCTAAACATCTTGCAAGGTTTTGACGTTAATTA |
| | 45887000 | CTATTATGATTGCTTTCTGTGTGTTACTGTTTTCCCCACACTTTAGCCAGCTAATGTGGAGCTACAGAAGGCCCTCGCCCCTACCCCTCC |
| | | AGATGTCCCAGCCCATGACAAGCAGGAAGGCCGGGTGCTGGGAGACTTCCTGGGGCTGGATCTGACATCATTCCAAGCAGATGATAACCT |
| | | GCCTTCCCGATTTCCAAACCCACAGCAAGACACCCTGGAGTTATTTATAAATGCGAGCCCCTGGGTGCACTTCTGACGGGACCAGCACCC |
| | | TGACGGCCATGAGAGGGTGGAGACAGCGCACCCCGAGCTCAGGGAGGCAGGAAACTCTGGACCTGGAGGCCGGGCACCATGAGGGACACG |
| | | CTGCAGGCCCAGCTGCTGCCGCCTGGGGCGGGGCTGCCCTGCAGGCTCCGGGAAAACCCAGAACCAGGCCGGATCAGCGTGTGTCAAGAG |
| | | GCGGGGCGTGAGAGATGAGCTGCTTTTTTTCTTCACAGGGTTGGCAGGAACTGCAAATAATAGAAAGTCTTTAGGGTCTAACACGCTGCC |
| | | CTGAAAACACTATCATTACTTTCCTAATGACTAACTGTGTCTTTCAGCCGGCGGGGCAGGCAGCTGAGGCCGCAGGCTCCCGCAGAGGAC |
| | | CGGGGGAGGCTGGCAGCCTGTAATCTGGGGGCGCTGACAGTGCTCTGCCCAGACCCTCGCGCCAGCTCCAGCTCCAGCACAGCAGCCCT |
| | | GGGTCCCTCTGGCCCCCTGCCCGCAGAGTCCAGGTGTGGCAGAGGCCGCCCAGTATCCCTTCTCCTCCTCCTTTTCTAAAAACAGAGTCT |
| | | CACGATGTTTCCCATGCGGGTCTCCAACGCCTGGGCTCAAGCGATCCTTCTGCCTCGGCCTCCCAAAGCGTTGGGATTAAGGGGCGAGCC |
| | | ACCGCGCCCGGCCCACCTTCCCTTCTGGTTCATTTCCAGTAAGGTCCTGTCCACAGCGTCCTTCCCAGCATTCCCACCAGGCTGCAGGCC |
| | | TTGGCCTCCCTCCCCTCCATTCTCATTCTCCCCGAAACCGCCAAGCGCGTCCAAAGCACGGGTTCGCCAAGCGCCCCCCCCGCCCCACTC |
| | | CACATTCCCTTCCCCGCCGACTCAGCCTCCGTAGCTCGCGGACGGCCCCTCCTCACGCCAGCCCAGGCTTTTTTTTTTTTTTTTTCTTCT |
| | | ATTTTAAGGTTGTCTTTTAATGACACAAGCGACATTTGGAGACAAAAGGACACATCTCTTCCTGACCCACCTCCAACCCCAGCTGACGGC |
| | | CGCCCTGAGCCTGGCGTAGACGGCCCGGAACGTTCCCTGCGTGGGTTCCGTCCATCCCGAACCCCTGTCCCCGCGCCGGCTCCGGGGGTG |
| | | CTCGGGGGGCCGCGTGGGGTCTGTGACGTCGCCTCGAGGCTGCATCCCGGTGACCCGGCAGCCCCTGGCGCTCGCGGGAGGCGGGCGGGC |
| | | GCGGACCCCAGGCTTTAGGGCGCGATTCCTGCAGCTGGCTGCCGGCCCGAGGTTCTGGGGTGTCTGAGGTCTCGGGCGGGGCGAGGACG |
| | | TTTCTCCGGCTCAGCCCCCCCACCTCCTGCCCTGCCGCCCCCCACACCCAGCTCCCCACGGACGCCAAGAGGCGCCTCCCACCCCGGCG |
| | | AGGACCCGCGGGGAAACGGGGCCCAGGCGCGGCGACTGCGGAGGACGCGCCTCGGCCCCAGCGCCCTGGTCCTCGGGGCGTCCGGCT |
| | | GCCCTTGCCCGAGGCCGGGGCGGGCGCTCAGCGCCGCGGAAGAAACGCCCGGGCGGGGACGCACAGCGAGGCGGGCTCCGCGGGAA |
| | | GTACCGGGAAAACGGCGCGGAGCGGAACAG |
| 249 | PCBP3 | TGGAGCAATCCCAGAGAGGCTGAGGTGTTCAGGCTGGCCCCAGATGCACACGAGCGTGAAGCCTGTTCAGAAGCCAGCTCCTCACACCCT |
| | | CTCCCCTGCCAGAGGCTCCAGCACCCCCTCCCCTCTCCTCTCCCCTCCCTTCCCTGTGGTCCTCCTGCCCACCCCACCCCCGTCTGCATG |
| | | TGCACCGTCACGGAGATGCGTGTACTAGGGCGGAGGTCGGGGACAGTCGTCAGAAGGACACAGGAAAGAAGGGAACAGGAATCCCATAAC |
| | | AGAACATTATCCGGCAGGAGTAATTAACACAGGCAGGACTGGAGGCTTTGTTTTGTTTTGCTTAAAAAACAGTGGTATTTAAATTAATGG |
| | | GCATGGGAAGACTATTCAGTGAAAGACATCGGTCATTGAGGTATCTATTCAAAAACACGGTTTAGTACTCTGCCACACACCGAACGCAAC |
| | | GCCACAGCAGCCATAGAAGCGTGTGTGGCTGTTTAACGTGGTCTTTTTGGGGAGGGCATCCTAGGCAGAGCAGGCGTGGAAGGGAAGGCG |
| | | GCGGACGGAACAAAACGCGGGCACGCAACGGCTGCTGCGCCGGATCTGAGGCAGGGCCAGCCTGTGGGAGCAGCAACATCGCTCGCAGGA |
| | | CAGCGATGGAGCCCCCACGAATCCGCGTGAAAGCAGCAACCACCTAGAAATGAACGTACAGCTGCTTAGAAACAGAATACGGATGACCCG |
| | | AAAGACTTCCCGATGGTAGTCACCAGCATACAGGACCTGACACGGGCGTGCGGGCAGGGTGTGCCGCTACGGGGTCCCTGGCGCACCTGC |
| | | TACCCCTGCTACCCGCATTCACCGCACGCGGAGGGTGCGGGCCGTGAAGGTTATACATGCAAATATCCTTCCACCAGCCAGTTCTCCTTC |
| | | CAGGAATCTGCCACCCGACCCTTGTGTTGTGCACAGACATGGTCCAGGTGTTTGCGACGTGATTGTTTATCAGAGAGAGAGAAGGGAAAT |
| | | CTCCAGGCTCGCTGTAGCTGCAGGAGCTCTGGGGGCTGCGCCCATCGTGGAGACGGATAGCTGTCTCTCATGAACACAGGACAGCAAGTC |
| | | CGGCTGCGGCCACAGAAGACTCGCCCTCCTGGACGCAGCGTCTTCCTTCCTCAGCCCCACACTGGAGGTGGCCAGTGCCATCCACAGCAG |
| | | AAGGGGCCAGCCGGGACCAGGCTCACGCCGTGGAATTCTGCTCTGTGGTAAGAGGAAGAGCGATAGCTGGAACCCAGCGCCGTCGCACA |
| | | CACAGCGGGGAAGAGTCTCAGAAATGTTACTTTGAGTCAAAAAGCTGGACAAAAAAAGGCGCAAGCCAGATGGTGCTGAAGAGGCCACAG |
| | | GAGGCTGGCAGCCAGGGGGTCTGGCACCTCACTCGGAGGCGCAGTGGGCCCGTCCGGAATTAGTGGCCATACGGCAAGTGCCGAGTGGAC |
| | | ATCAAACCGTCACTTCAGACTCCTGCGCTTCACTGCCTGTCGGTTATGCCTGGGTTTTGAAATCAAGTCACAGAACACCTGGAATGTGGT |
| | | GTTTACGCAGAACAAAGCGGGTGCCTCGGAGGAGAGAGCCTAGGGACAGGGGCACCTCCCGGTGTGGGTGCCCAGGGTTGCAGGGTGGCT |
| | | TCCTCTGTCTGCGCGGTTTTCAGAGCCCCAGGGTCCTGCCTGCCCGGCTGCCTGGAGGCGGCCCACATCCTGCTCTGCGCCGCCGAATCT |
| | | CAGCCTGAACAGCTTCGCTGGTGTTTGTGTTGACTTATTTGTTCTTTTTTTTTTTTTTTTTTTTTAAATAAAGGATTCCGATGCTGTTAC |
| | | AGTCAATAAAAGCCACAGGTCTGGGTGACCTACAAATGTGTGTGTCTGACTTTCTGCAGTTTAAATCGCCACTGAGCCTTAAGGCGTCTG |
| | | GCCCGCGCATTGAGGAATCCACGTGGGTCTCGGGGTCCCCATGCCTGCCCAGCTCCCTGCTTCAGCCTGGGCGGGTCTGGCGGGCATTTC |
| | | TGCGAGCCTGTCCCTGGGCCCGCCTCCTGGCCAGACTTCCAGAAACATTGTCCACATCCCCGTTGCACGTCCCCCCGTCACCGGAAACTG |
| | | CAGCCCACAGCACTGGGAAGAACCCGGGAGGCAGGCGTTAGGACGGGGTGGCCGAGACAGGGAAGGGAGCCATGGCGGACGTCCTCACCC |
| | | AAGCCAGGGCTTCCTGCCCCTGTGGTACTGACAGGAGCCCCGCAGGACGTGGGGTTGGCTTTGGGCAGCTCGGTGGACACTTCTCTTTCA |
| | | GATCCTGCCACAGCAAAGCTCACGAGACTCACTTCTTCCCATTGGAATTCACTAAGAACAAATTCAACAATTCAGACGCCCCAGCTGGAG |
| | | GTTTATTTTATGGATTTTACCTGTGCGGTATTTAGGGTTGTGTTTATGAATAAAGGTGTGCGTTCTGGCAAGTAGAAATACAGAGCTTGT |
| | | CTTTCACCCAAGTATCTGTAACTTTCTCCAATGCAGACACTAAAATGCAATAAAAACAAACCAAACCCATTAAACATGAATTAGATGAGG |
| | | CAGGCTGATGGGAGGTTGTGGGATTAACAGGCCGTCAGCGGATTGAAGCTGCGCACATCGCTGGGATGCTGCTGCGGGAGGATTCGGTCT |
| | | AATCCGGGAGCATCTGGCTGGGCAGTGGGCAGCGTCTGCAGTCGTGGCTGCTTGAAGGTATGAAGGTTGTGGCCTTTGCTTCCCCCCATC |
| | | AGGCTGCCCCACCCTGGACCCCACCCAGACCCCTCGGGCACCCTGGGGTCATCTTCAGCTCCCCCTTCTCTTCCTTCCTTCTCTTCCGCC |
| | | TGGGCCCCTACTGTGACCCGAGGTCAGCAGAGGACCCTGGCAGGTGGCTGCTCCCTGGGACTCGACTGTGCAGGTGAGGCTTGGGGTGAC |
| | | CGCTGCTCCTGCTCCTGCTCCTCTCGCCGTCCCCACCCTCCTCCATCATGCTGTCAACATGCATGTGGGCTGCAGCCCTCAGCCTGCAGG |
| | | ACGCTGTCAGTGCAGCTCCTCAGTGGCCAGG |
| 250 | PCBP3 | ATCTTGTCTTCCTTGTCCCAGTCCTGGAACCAGCCACTGCCCCAGCAGCTCCTGTGTGTGGTGGCATGTTCTGGAAGCCAGGATGCATGG |
| | | TGCTCCTGGGCTGCTGTGGGTCCTGGGCTGCTGTGGGTCCCGAGCTGCTGTGGGTCCTGGGCTGCACCCCTGCAGAACACTTCCTTCCAT |
| | | GTTCAGCTCCCTATATGGAACCCCAGTTCCAGCCCCACAGCACAGGGTCCCCCAGTTCTTCCTGCCTCAGGTGTGCACCACGAGGAATCC |
| | | AACTGCCAGTATCTGTGCGTGGCCTCCCGCCGGGAGGAGGCTGCCGGAGGCTCTGAGCTCTAGCCCCACAGCACTGGCACATCCTAGATT |
| | | TCCGGGAAGACACGGCCTCCTCCCCAGGGGAAGGTGGTGGTGCCCACACCCAGAGCATTCATTCCTGCAGTGGAGACAGAGGGACCTGCC |
| | | TCTCCAACTGTGGGTGTCAGGAGCCAAGGCGCATGGTAAATGGGGCTCTCTGTGAGGCCAGGTGCACGGCCCCATCTCCAGCAGCAGCG |
| | | GCCATGCCACCCAGCTGCACTCTGTGGGGGAGGTGCCATGATTGACGGGGGCCCCTCCCTGTGTCCAGTGTCCTCCTCCCTCCACGGGCC |
| | | CCTCTGCACACCGTCCTCACAGTCTCCCTCTGCACACCGTCCTCACAGCCTCCCTCTGCACACCATCCTCATGGTCTCCCTCTGCACACC |
| | | GTCCTCACAGCCTCCCTCTGCACACCGTCCTCACAGCCTCCCTCTGCACACCGTCCTCACAGCCTCCCTCTGCACACCATCCTCATGGTC |
| | | TCCCTCTCCTTCCACAGACCCCTCTGCTCGCCATCCTGACGGCCTCCCTCTCCCTCCACGGACCCCTCTACACACTGTCCTCCCAGCCTC |
| | | CCTCTACACGCCATCCTCACAGCCTCCCTCTCCCTCCACGGGCCCCTCTACACACCGTCCTCACGGCCTCCCTCTCCCTCCACGGGCCCC |
| | | TCTGCACACCGTCCTCACAGCCTCCCTCTCCCTCCACGGGCCCCTCTGCACGCCGTCCTCACGGCCTCCCTCTGCCTCCACGGGCCCCTC |
| | | TGCACGCCGTCCTCACGGCCTCCCTCTGCCTCCACGGGCCCCTCTGCATGCCGTCCTCACGGCCTCCCTCTCTCTCCACGGGCCCCTCTG |
| | | CACGCCGTCCTCACGGCCTCCCTCTCTCTCCACGGGCCCCTCTGCACGCCGTCCTCACAGCCTTCCTCTTTTTCCACAGACCCCTCTGCA |
| | | CGCCGTCCTCACGGCCTCCCTCTCCCTCCACGGGCCCCTCTGCATGCCGTCCTCACAGCCTCACCGACGTCACCATTGCTGGCCCCGCTT |
| | | CAGGTGACAGGCCACAGTAGCACCTGTCAGCTCTGTCCCGCTGCTGGACAGGGAGATACTGGGCCACTCAGCCCAGCGGGGAACGTGTGT |
| | | CCCGAAACTGCCTTGGGCTCGCCATCAGAACTGTGGCAGCATCTTCCAGCGTTCCTTTTAACAGGCTGCCGTTGGAATAGGAGTCACGGA |
| | | GCAATTGCAGTGCTAAGTTTTCTTTAAGTCACACAATTGAAGGAGGCTTTATTTTTCACACATTTCTTCCAGAGTTTCCTGGTAGCCTGA |
| | | GTGCATGGGTGATGCCCCCTGAGTTATTTATCAGGGGCAGCCAGCTGCCCTCCCCCGGGGCACTTACAGTCAGCCCATCTCTGTCCTGGT |
| | | CAGGTGGGCGCCAAGGAAGACCCGGCTCAGGGCCTCTGTATGGGCAGCCTGGCTTGTACACACACCCCTCCCCACCAGCAGATTCTGAAT |
| | | TCTCCCTTCTTCATGCACACCGGGAAGGTCCCTTCTGCACTCATACCGGGAAGGTAGGCAGGTTTCGGTAGTGTCTGCCTCCAGTGTTTT |
| | | CCTCCTCCTGCTCTATGACATCATCTTTCTGTGATTTTTTTTTTCTTGCAGGAAGTTGGAAGCATCATCGGGAAGGTAATTATTGATTGA |
| | | ATCTCTGCCTCTCCTGGGGTCTCTGTAAGGGGATGGTGAGGATGGCAGCCTCCCTGGGTACTAGGTGGCACCCAGTAGGTGCGCCTTTCC |
| | | CAGTTGGTGGGTGGTCTGTGTTCCATGAAGACAGGACCCCAGAGGTGTCGCCTTTATGCTGTATGACATTGAAGCTGGTCCCTGGCTCTG |
| | | CGTGGCCTGAGGGGAAGGGGTTCACTCCAGCTGGTCACCTCGCTGCCCCCTGCCCGTGGCCTTGGTGGCCAGTCCTTCTTTCCCGGTTGA |
| | | AGACCCCACGAAGAATGATTTCTCACGCCTTCTTCAGCCGGCTGTGTAGTCTGGGTGGTCTCCAGGAGTGCCAGTGGAGGCAGCAGCCCC |
| | | CAGACAATTCCTTTCCAAATCAGGGCTGGCCCGGGGGAAGTAAGGCCCAGTTTGGAAGCCTGCTGCCCCGGGAGGCCGAGCAGTGAGGGC |
| | | CACCTCCCTGTCTTCATCACATTTTCACCGCTTCCGGGGGTCCTTCCCCTCAGTCCCACCATGGGGGCGCC |
| 251 | COL6A1 | GCTGGACACCTCTGAGAGCGTGGCCCTGAGGCTGAAGCCCTACGGGGCCCTCGTGGACAAAGTCAAGTCCTTCACCAAGCGCTTCATCGA |
| | | CAACCTGAGGGACAGGTAGGAGGGACGCCCCGTGACCTTCCTCCTGTGCTTCTGGGCCTCTTGGAGGGAGGGGTGGGGGCCCAGGGGA |
| | | ACACGGGTGCGACGGCCTCAACCTCCTAAGGTTGGGCGAGCGTTGCCCTGACCGGGGCCCCTCCCGGCGCCCTCCAGAGTGAGGCCGG |
| | | GGCCCTTTCCGGCGCCCTCCAGAGTGAGCTGGTCTGAGCCTCTCCCAGCGCCTTCCAGAGTGAGCTGGTTTGAGACCCTGCTCGCGGGG |
| | | GTGGCACCTGTTCAGCAGGGCCGAGGTGACAGTGAGGCTGAGATGTAGGGAAGAGAGGCTCCCGCAGGCTGACCGAGAGGGCTCAGCG |
| | | CACTGGCCCAGACACGCAGTCCTGCCTGGTGCGCGGGAGCCCCTCACTAACCACCTGGACCCTGGTTTGTTCCGTGGGCAGTGAGAGCC |
| | | TCTACCTGGGTCCTGGATCCCACGTTCTGAAGGTCCCCGACTCGGGAGCCAGGAGGGGTGTCGCTCTGCAGCCCCAGGGCCCCCAGGCT |
| | | TGGTTCTGGGCTTGGGACACGGCACCCTCTGCTCCACGTTCCTCCATCTGTGCGTGTGGCTGAGGACAGACCGGGGGGAGAGGGGAGTC |
| | | GGTCCTGTGGGTGCACAGGGCCGCTGAGGGGGGGGCATGTAGAACGGGGCTCCCCCACTGAGACGGGTCCTGGCAGTGGGGACACAGC |
| | | TTAGCCGGCGTAGGAACCCCCGTCCTCCTTGACCCTGCTGACTGGCCGCTGGGCCGGAGCCTCCCGCCACCAGAAGGGGCACAGTCAGA |
| | | GGCTGCCGGTAACAGCAGGGTGGACCTTCCAGCCCACACCGTGCCCAGCAGGAGCCATTGGTACCAGGAACCCTGAGCTTAGTGGACAT |
| | | GGCCAGGCCCGTGCGGCAGTGTTTGGGGGGGGGTCTGGCTGTGGATGGCACCGGGGAGGGGCGGCCGCGTGGCCCAGCGTCCCCCGAG |
| | | TCGCCCTTGTTGCCTTTACTCAGTCTCCCCATGACTCAGTTTCCCACCTGTGAAATGGGGCGGAGTCATCCCCATGTCGCTGCCACTGGA |
| | | TTCCTGCAGGCGCCGTGGTCACTCTGCTGAATGGATGGGAGGGTGGGTGGGGCAGAGGTGGGCCCACCCCAGGCTGGGGCAGAGCAGA |
| | | CCCCTGAGAGCCTCAGGCTCAGGTGCTCAGAGGGCAGCGAGGGGGCTGCTCAGATCCCCGGGGTGCCTCCTTCCCCCACTGTCATGCTG |
| | | CCCCACTGCAGGCCCAAGGACCCCACCCCAGCAGGGCCACACACTCAGGGCTCCTGGTCTGAGGGCCTGAGGGATCGGGGCGCAGGTC |
| | | GCTTGCTGGCCACACCCGCCTGCACAGCCTTCCAGGAGGGCCGGCCTCAGGGCCACAGGGCAAGTCCAGCTGTGTGTCAGCCACGGCCA |
| | | GGGTGGGGCAGCCTGTCCATCTGGGTGACGTCGCGCCCTGGGACGGGTAGCGATGGCGCCAGGGGCCGCCCGCCTCACGCCCGCCGT |
| | | GCCTGTTCCTGGCAGGTACTACCGCTGTGACCGAAACCTGGTGTGGAACGCAGGCGCGCTGCACTACAGTGACGAGGTGGAGATCATCCA |
| | | AGGCCTCACGCGCATGCCTGGCGGCCGCGACGCACTCAAAAGCAGCGTGGACGCGGTCAAGTACTTTGGGAAGGGCACCTACACCGACT |
| | | GCGCTATCAAGAAGGGGCTGGAGCAGCTCCTCGTGGGGTGAGTGGCCCCCAGCCTCCTGCCCACGCCAGTTCTCACGCGTGGTACCCAG |
| | | CCTGGGCTGGGGTTGGCCTGGGGTCCCTGTGCGGCTTCAGCTGCAGCCTCCCTGTTCTCTTGGAGGCTGCACGGCCTCCCTGACCCACTT |
| | | TGTGGGCAGGAAAGAGACGGAGACAGACAGAGACAGAGAGAAACAGAAACAGGGAGAAACAGACACAGAGAGAGACAGAGACAGAGAGA |
| | | GATAGAGACAGAGACAGAGAGAGACAGAGACAAAGAGTGACAGAGGGACCAAGACAGGCAGACAGAGACAAACAGAGACAGAGACAGAG |
| | | ACACAGAGAGAGACACAGAGAGACAGAGACGGGAACAGAGACAGGCAGACAGAGACAGAGAGAGACAGAGACAGAAACAGAGACAGAGG |
| | | GACAGAGACAGGCAGAGAGAGACAGAGAGACAGAGACAGAGACAGACAAACAGAGACAGAGAGACAGAAACAGGGACAGAGACAGAAAG |
| | | AGAGAGAGACAGAGGGAAACAGAGAGAGACAGAGACAGATAGAAAAAGACAGAGGCAGAGAGAAGCAGAGACAGAGAAACAAAGACAGT |
| | | CAGAGACAGACAGAGACAGAGACAGAAACAGAGACAGAGAGACAGAGACAGAGGGGCAGAGACAGGCAGACAGAGAGACAGAGACAGAG |
| | | ACAGCGAAACAGAGACAGAAACATACAGAGACAGAGAGACAGAGAGAAGCAGAGACAGACAGAGGCAGAGAGACAGAGAGAAGCAGAGA |
| | | CAGGGACAGAGACAGAGACAGAAATAGAGAGATAGAGACAGAGGGACAGAGACAGAGAGATAGAGACAGAGAGGGAGACAGAGAGATAG |
| | | AAGCAGAGAGAGAGAGACAAAGACAGAGGCAGAGAGACAGAGAGAGAAGCACAGACAGAGACAGACAGAGAGACAGGGACAGACAGAGA |
| | | CAGAGAGACCGGAAACAGAGGCAGAGAGACTGAGAGACTGAGAGAGACGGGGTGGTTTTCCCCACAGCATCAACACCAAGCAGGGCTAG |
| | | GATCACTGAAACAGACTCATCAGACCCGAAGCATGCGCTTTCTCGGGGTTTTTCTGGACTGAGGGGTTTCCTCTCATCCCAGTGTCCAGC |
| | | TGTGGGGACGCAGGGGCCGCAAGCCCCGGAGTGTCCAGAGGGGAACGTGGCCTCCCCACACCCAGCCCTTCACGAGGCCTCAGGATCCC |
| | | AGTGGGGGTACCCGAGGCTGCCCTGTCCAGCCAGGCGGTGCGGGGGGTTTGGGGAGAGCCTCTCCCCGAGGTCGGTCTCAGAGGGCCA |
| | | CATGGCCGGTGTGGGCCGGACATTCCCTTTCCAATGGTTGTGCCCACTTCCCTCCAGAGTTGGTGCCAAGCTGGGACCTGGGGGACTTGG |
| | | AGTCTCAGGAAGTCGTCCGCTGTCTGCAGGGGGTGCATGGGGGATGTGGCCACACACGTCAGAGTGCGGCCCCCTGTGGAAGCCACAGA |
| | | CAGACACGACTCCCCTAAATGAGCTCGCCCTTCTGGCCGAGATGCTCAGCGTCCCCAGCAGGCTGCCCGACTGCCCTGCGATACTGCCCT |
| | | CCTTCCTGCTGCTCCCACTTTCCCTTTCGGGGGGTTGGATTTGGGGCATTCAGGGATCGCCCTGTTGTTTGCTCATCACACCCATTTCCT |
| | | GCAAGAGCCACGGTGACCGAGCAGCCTTGAGTTGAGGCAGCTTGTGGGTAGACGCGGCGGGCATCTCGGAGGGGCACGCTCCCTGCCACC |
| | | CTCAGCCTCCACTCACTGGTCAGGGGCTTTGCGCCCCAGGGCACCCCAGGAACCGAGCCTCCTTTGGGGTCATGGGTGCCTCTCCTGGGA |
| | | GGGCGTGGATTTTCCAAAGCAGTTTAGAGAAATGAGACCCACAGGCGTTATTTCCCATGGTGAGGTTCTTTTCAGTAACCCCCACCGTAT |
| | | AGCCAGGATCAGCAAAGAGAGGCGGCTCCTCCCGGTGAGACAGGGACCAGCACCTCCCGGACAGGCTTGGGTCTCCCTCCAGTTCCCCCA |
| | | CCTAGTCTCGAGGTCTCACGCTGCCCTCTCCTGTCCAGGGGCTCCCACCTGAAGGAGAATAAGTACCTGATTGTGGTGACCGACGGGCAC |
| | | CCCCTGGAGGGCTACAAGGAACCCTGTGGGGGGCTGGAGGATGCTGTGAACGAGGCCAAGCACCTGGGCGTCAAAGTCTTCTCGGTGGC |
| | | CATCACACCCGACCACCTGGTAGGCACCGGCCCCCCCCGGCAGATGCCCCCAACCACAGGGAGTGGCGGCTGCAAGGCCCCCGGCAGC |
| | | TGGGACCGTCTTTTGGTCCTCGGGAGGGTGTGGGTTCTCCAGCCGGCCACCCTTGCCCCTGAGAGGCCAGCCCCTCCTGCTGAGGAGCC |
| | | TGGAGCGCCCCAGCCCAGCCTCCCCTCTGGCCCTGTGGGAAGCGGCCCCGGCCGTCAGGGGTCCCAGCCCTGCTCAGCCCACCCTGAA |
| | | CACTGCCCCCAGGAGCCGCGTCTGAGCATCATCGCCACGGACCACACGTACCGGCGCAACTTCACGGCGGCTGACTGGGGCCAGAGCCG |
| | | CGACGCAGAGGAGGCCATCAGCCAGACCATCGACACCATCGTGGACATGATCGTGAGGCCCCTGCCCAGGAGACGGGGAGGCCCGCGGC |
| | | GGCCGCAGGTGGAAAGTAATTCTGCGTTTCCATTTCTCTTTCCAGAAAAATAACGTGGAGCAAGTGGTAAGAGCCCTCCCCACCACCCCC |
| | | AGCCGTGAGTCTGCACACGTCCACCCACACGTCCACCTGTGTGTTCAGGACGCATGTCCCTATGCATATCCGCCCATGTGCCCGGGACAC |
| | | ATGTCCCCTGCGTGTCTGCCCGTGTGCCCGGGATGTGTGTCCCCCTGCGTGTCCACCTGTGTGTCTGCCCATGTGCCTGGGACATGTGTC |
| | | CGCCTGTGCGTCCATCCGTGTGTCCGTCTGCCCATGTGCCTGGGTCGCATGTCACCCTGTGTCCCAGCCGTATGTCCGTGGCTTTCCCAC |
| | | TGACTCGTCTCCATGCTTTCCCCCCACAGTGCTGCTCCTTCGAATGCCAGGTGAGTGTGCCCCCCGACCCCTGACCCCGCGCCCTGCACC |
| | | CTGGGAACCTGAGTCTGGGGTCCTGGCTGACCGTCCCCTCTGCCTTGCAGCCTGCAAGAGGACCTCCGGGGCTCCGGGGCGACCCCGG |
| | | CTTTGAGGTGAGTGGTGACTCCTGCTCCTCCCATGTGTTGTGGGGCCTGGGAGTGGGGGTGGCAGGACCAAAGCCTCCTGGGCACCCAA |
| | | GTCCACCATGAGGATCCAGAGGGGACGGCGGGGGTCCAGATGGAGGGGACGGCGGGGGTCCAGATGGAGGGGACGGCGGGAGTCCAG |
| | | ATGGAGGGGATGGCGGGGTCCAGATGGAGGGGACGGCGGGGTCCAGATGGAGGGGACGGCGGGGTCCAGATGGAGGGGATGGCGGG |
| | | GTCCAGATGGAGGGGACGGCGGGGTCCAGATGGAGGGGACGGCGGGGTCCAGATGGAGGGGACGTCGGGGCTCCAGATGGAGGGGAC |
| | | GGCGGGAGTCCAGATGGAGGGGACGGCGGGGTCCAGATGGAGGGGACGGCGGGGTCCAGATGGAGGGGACGGCGGGGTCCAGATGGA |
| | | GGGGACGTCGGGGCTCCAGATGGAGGGGACGGCGGGAGTCCAGATGGAGGGGACGGCGTGGTCCAGATGGAGGGGACGGCGGGGTCC |
| | | AGATGGAGGGGACGTCGGGGCTCCAGATGGAGGGGACGGCGGGGGTCCAGATGGAGGGGACGGCGGGGTCCAGATGGAGGGGACGGC |
| | | GGGGTCCAGATGGAGGGGACGGCGGGGTCCAGATGGAGGGGACGGCGGGGTCCAGATGGAGGGGACGGCGGGGTCCAGATGGAGGG |
| | | GACGGCGGGAGTCCAGATGGAGGGGACGGCGTGGTCCAGATGGAGGGGACGGCGGGGTCCAGATGGAGGGGACGTCGGGGCTCCAGA |
| | | TGGAGGGGACGGCGGGGTCCAGATGGAGGGGATGTCGGGGTCCAGATGGAAGGGACGGCGGGGTCCAGCAGGCAGGCTCCGGCCGTG |
| | | CAGGGTGTGGACTGTCCCGGGGGCGCTGGGGGCTTCTGAGGGTGTCTCTGTCCGCCCTGCCCTCAGCCGCACTCTGTTCAGAAGGACCT |
| | | TTCTGGAGGTAGGAGGGTGAGAATGTGGGTCCCCTGCTTCTGTGTGGCTCAC |
| 252 | COL6A1 | GGCCGGGGAGGCGGGGAGGCTGCCCCAAGAGTAAAAGCCTTTCTGACGTGCGCAGGACGCGGCCCTGACTGGTCTAACTGACTCTTTCT |
| | | CTTCTCCTCAGCTTGCTGTGGTGAGACCCAGGCTCTAGCTCCTGAGAGAATGGATCCCGGGGGTCGGGGAGCGAGGCCTGGGTCCCACA |
| | | CATGTCACAGGACAGCACATGGCACTCTGGTCCCCGCCCGCAGCTCCCTGCACCTGCCCGCCCCCTCTGGGGCCTGCTCCAAGCCAGCA |
| | | GGGTTCCCGGGTGTTGGGCTGGGCCCCGCCCTCTTTCACCCATAACTGAAATAACCAGGAGCAGGCTTGGGGGGGTCCCTGCTCCATCAT |
| | | TCTGGCCCACAGGCCCCACCCTAGCCTGGCTGAGCAACGCCAGCCCTGACCAGCCGCCGGACAGAGCAGCCTTTACGGGGCCATGGGAG |
| | | GGGGTGGGCTTTTCTGGGGCTGAGACGGGGGGACCCCAACGTGTCAGGTGAGGATGTGGCAGCCAAGGAGGGGCCAGGGCGGTGGAG |
| | | GGGAGGGGCCAGGGCACTGGAGGGGAGGGGCGTGCTCTGCTGACACCGCCCCCGCCTGCAGAATGCAAGTGCGGCCCCATCGACCTCC |
| | | TGTTCGTGCTGGACAGCTCAGAGAGCATTGGCCTGCAGAACTTCGAGATTGCCAAGGACTTCGTCGTCAAGGTCATCGACCGGCTGAGCC |
| | | GGGACGAGCTGGTCAAGGTGAGGCCTCGCCCCGCCCGGCTTTCTCAAGCCCAGGTGCACCCCGACCCTGCCGGCCGCCCCTGCCCGCG |
| | | CCAGACCTCAGCCTCCCGAGGCCACCGCTGCATCCCTGTGACTTCCCTACTCATGACAAGGATGCCAGGCACGCGCCAGCCCGTCCAGG |
| | | CCTCCAGCTCCACCTGGCGAGGCTGGCCCATTGTACACAGGCGCCCCAGATGAGGGAGGGTCTCCCCCTCTCCTTGAAGGGCGGTAGTC |
| | | TGGGGTCCTGAGTGCTGGGTGTGGGCTTGTCCCTCGTGGACAGAACCCAGGAGGGCTTCATCCACCAAGGAAGATTGCTTTGCAGGGTAC |
| | | CCAGGTCCCGGGGGCTGTGCCACCCTCTGGGCACCCGGAGCCAATCGCAGGGTACCCAGGTCCCGGGGGCTGTGCCACCCTCTGTGCA |
| | | CCCAGAGCCAATCGCAGGGGACCCAGGTCCTGAGGTCCTGGGGGCCATGCCACCCTCTGGGCACCCGCAGCCAATAGAGTCACCCTTGG |
| | | GAAGCTTATGCGGACCTGGGGCAGCACTCGCGTCCTGACCCCGGTGCCGGTCCCACAGTTCGAGCCAGGGCAGTCGTACGCGGGTGTGG |
| | | TGCAGTACAGCCACAGCCAGATGCAGGAGCACGTGAGCCTGCGCAGCCCCAGCATCCGGAACGTGCAGGAGCTCAAGGAGTGAGTGCCC |
| | | CACGCGGCCAGGACCCTCCCACCCCTCGCCCCGACCGCTGTTCCCACGGCAGGTCGGCCCTGACCCCTGATCCCAGGTGGGCTCGGCC |
| | | CCGCGGCAGGCCTGGCCCCAACCGGCCCTTCCTGCCCTTTGCTATGCAGAGCCATCAAGAGCCTGCAGTGGATGGCGGGCGGCACCTTC |
| | | ACGGGGGAGGCCCTGCAGTACACGCGGGACCAGCTGCTGCCGCCCAGCCCGAACAACCGCATCGCCCTGGTCATCACTGACGGGCGCT |
| | | CAGACACTCAGAGGGACACCACACCGCTCAACGTGCTCTGCAGCCCCGGCATCCAGGTGGGGTGGCCACCCCCAGGCTGCACCTGCCCC |
| | | GCCTAGGGCGCCCCGCCAGCCAGGGTGGCCTTGTCCCCAGAAAGACGAGGGCAGAGCAGGCTGCGCCACACCGATACTGTCTGTCCCCAC |
| | | AGGTGGTCTCCGTGGGCATCAAAGACGTGTTTGACTTCATCCCAGGCTCAGACCAGCTCAATGTCATTTCTTGCCAAGGCCTGGCACCAT |
| | | CCCAGGGCCGGCCCGGCCTCTCGCTGGTCAAGGAGAACTATGCAGAGCTGCTGGAGGATGCCTTCCTGAAGAATGTCACCGCCCAGATCT |
| | | GCATAGGTGCGCATGGGGCCACCCGGGCAGTCCCAGATCTGCGTAGGTGCGCGCGGGGCCGCCCGGGCAGTCCCAGATCTGCGTAGGT |
| | | GCACGCGGGGCCGCCCGGGCAGTCCCAGATCTGCGTAGGTGCACGCGGGGCCGCCCAGGGCCGTCCCAGATCTGTGTAGGTGCGCGCA |
| | | GGCGCCCAGGGCTGTCCCAGAGGCCTCCTCCCAGCTCACTGTTACCTCCAGGGGCACGGCCACCCTGTAGGTGCGCACGGGGCCGCCT |
| | | GGGGCTGTCCCACAGGCATCCTCCTCCCGGCTCGCTGTGACTTCCGGGGGCACGGCCACCCCTGTGCTCGGCCGGGAGGTCCTGTGACA |
| | | TCTCCTTGCGGGGTTATAGGTGGAGCAGTGGGCTCACACTGCACGGCTTTTCTCTTTTACAGACAAGAAGTGTCCAGATTACACCTGCCC |
| | | CAGTGAGTACCTCGGCGGCCGGGACACGTGGGGAGGAGGGCACCGTGGTTGGGGCGAGGGCTCTGAGAGGACGGGGCTCTGGGAGGAG |
| | | GGCCTGGCGGTCACGAGAGTAGGTGCATGGCTCACTCCGGTGGCTGAGCACCACCGTGCCGTGCCCTCTCTGGGGAGCTTAGACGCTCT |
| | | CTGGCCGGCCCACTGCGGCTGCATCACCAGGGCCTCATGCTAACGGCTGCCCACCCCGCCCCGCAGTCACGTTCTCCTCCCCGGCTGAC |
| | | ATCACCATCCTGCTGGACGGCTCCGCCAGCGTGGGCAGCCACAACTTTGACACCACCAAGCGCTTCGCCAAGCGCCTGGCCGAGCGCTT |
| | | CCTCACAGCGGGCAGGACGGACCCCGCCCACGACGTGCGGGTGGCGGTGGTGCAGTACAGCGGCACGGGCCAGCAGCGCCCAGAGCG |
| | | GGCGTCGCTGCAGTTCCTGCAGAACTACACGGCCCTGGCCAGTGCCGTCGATGCCATGGACTTTATCAACGACGCCACCGACGTCAACGA |
| | | TGCCCTGGGCTATGTGACCCGCTTCTACCGCGAGGCCTCGTCCGGCGCTGCCAAGAAGAGGCTGCTGCTCTTCTCAGATGGCAACTCGCA |
| | | GGGCGCCACGCCCGCTGCCATCGAGAAGGCCGTGCAGGAAGCCCAGCGGGCAGGCATCGAGATCTTCGTGGTGGTCGTGGGCCGCCAG |
| | | GTGAATGAGCCCCACATCCGCGTCCTGGTCACCGGCAAGACGGCCGAGTACGACGTGGCCTACGGCGAGAGCCACCTGTTCCGTGTCCC |
| | | CAGCTACCAGGCCCTGCTCCGCGGTGTCTTCCACCAGACAGTCTCCAGGAAGGTGGCGCTGGGCTAGCCCACCCTGCACGCCGGCACCAA |
| | | ACCCTGTCCTCCCACCCCTCCCCACTCATCACTAAACAGAGTAAAATGTGATGCGAATTTTCCCGACCAACCTGATTCGCTAGATTTTTT |
| | | TTAAGGAAAAGCTTGGAAAGCCAGGACACAACGCTGCTGCCTGCTTTGTGCAGGGTCCTCCGGGGCTCAGCCCTGAGTTGGCATCACCTG |
| | | CGCAGGGCCCTCTGGGGCTCAGCCCTGAGCTAGTGTCACCTGCACAGGGCCCTCTGAGGCTCAGCCCTGAGCTGGCGTCACCTGTGCAGG |
| | | GCCCTCTGGGGCTCAGCCCTGAGCTGGCCTCACCTGGGTTCCCCACCCCGGGCTCTCCTGCCCTGCCCTCCTGCCCGCCCTCCCTCCTGC |
| | | CTGCGCAGCTCCTTCCCTAGGCACCTCTGTGCTGCATCCCACCAGCCTGAGCAAGACGCCCTCTCGGGGCCTGTGCCGCACTAGCCTCCC |
| | | TCTCCTCTGTCCCCATAGCTGGTTTTTCCCACCAATCCTCACCTAACAGTTACTTTACAATTAAACTCAAAGCAAGCTCTTCTCCTCAGC |
| | | TTGGGGCAGCCATTGGCCTCTGTCTCGTTTTGGGAAACCAAGGTCAGGAGGCCGTTGCAGACATAAATCTCGGCGACTCGGCCCCGTCTC |
| | | CTGAGGGTCCTGCTGGTGACCGGCCTGGACCTTGGCCCTACAGCCCTGGAGGCCGCTGCTGACCAGCACTGACCCCGACCTCAGAGAGTA |
| | | CTCGCAGGGGCGCTGGCTGCACTCAAGACCCTCGAGATTAACGGTGCTAACCCCGTCTGCTCCTCCCTCCCGCAGAGACTGGGGCCTGGA |
| | | CTGGACATGAGAGCCCCTTGGTGCCACAGAGGGCTGTGTCTTACTAGAAACAACGCAAACCTCTCCTTCCTCAGAATAGTGATGTGTTCG |
| | | ACGTTTTATCAAAGGCCCCCTTTCTATGTTCATGTTAGTTTTGCTCCTTCTGTGTTTTTTTCTGAACCATATCCATGTTGCTGACTTTTC |
| | | CAAATAAAGGTTTTCACTCCTCTCCCTGTGGTTATCTTCCCCACAAAGTAAAATCCTGCCGTGTGCCCCAAAGGAGCAGTCACAGGAGGT |
| | | TGGGGGGCGTGTGCGTGCGTGCTCACTCCCAACCCCCATCACCACCAGTCCCAGGCCAGAACCAGGGCTGCCCTTGGCTACAGCTGTCCA |
| | | TCCATGCCCCTTATCTGCGTCTGCGTCGGTGACATGGAGACCATGCTGCACCTGTGGACAGAGAGGAGCTGAGAAGGCAACACCCTGGGC |
| | | TTTGGGGTCGGGAGCAGATCAGGCCTCAGTGGGCTGGGGCCGGCCACATCCACCGAGGTCAACCACAGAGGCCGGCCACAGGTTCTAGGC |
| | | TTGGTACTGAAATACCCCTGGGAGCTCGGAAGGGGAGTTGAGATACTGCAGGGCCCATAGGAAGAAGTCTTGGGAGGCTCCACCTTTGGG |
| | | GCAGAGGAAGAAGTCTTGGGAGGCTCCACCTTTGGGGCAGAGCAAGAAGAGGGCGGAGGGCAGAGGCAGCGAGGGCTCATCCTCAAAAGA |
| | | AAGAAGTTAGTGGCCCCTGAATCCCAGAATCCGGGGTGCACGGCTGTTCTGGGGGCCGCTAGGGGACTAAGAGGATCGGCCGAGGGCTGG |
| | | GCTGGAGGAGGGCAGCAGGGATGGGCGGCGAGGGTGAGGGTGGGGCTTCCTGAAGGCCTTCACCTGCGGGGACCCCGGCGAGCCCCTCAG |
| | | GTGCCACAGGCAGGGACACGCCTCGCTCGATGCGTCACACCATGTGGCCACCAGAGCTGCGGGAAAATGCTGGGGACCCTGCATTTCCG |
| | | TTTCAGGTGGCGAACAAGCGCCCCTCACAGAACTGCAGGTAGAGACGGGCCCGGGGCAGACGCAGTGAGGCGGTGGGCGGGGCCCGGG |
| | | GCAGATGCAGTGAGGCGGTGGGCGGGGCCCGGGGCAGAGGCAGCGAGCGGTGGGCGGGGCCCGGGGCAGACGCAGTGAGGCGGTGG |
| | | GCGGGGCCCGGGGCAGAGGCAGCGGGTGGTGGCCGGGGCCCGGGGCAGACGCAGTGAGGCGGTGGGCGGGGCCCGGGGTAGTCGCA |
| | | GTAGGTGGTGGGCGGGGCCCGGGGCAGACGCAGTGAGGTGGTGGGCGGGGCCCGGGGCAGACGCAGTGAGGCGGTGGGAGGGGCCC |
| | | GGGGCAGACGCAGTGAGGCGGTGGGCGGGGCCCGGGTCAGAGGCAACGGGTGGTGGGCGGGGCCCGGGGCAGACGCAGTGAGGCGG |
| | | TGGGCGGGGCCCGGGGCAGATGCAGTGAGGCGGTGGGCGGGGCCCGGGGCAGATGCAGTGAGGCGGTGGGAGGGGCCCGGGGCAGA |
| | | CGCAGTGAGGCGGTGGGCGGGGCCCGGGGCAGACGCAGTGAGGCGGTGGGCGGGGCCCGGGGCAGACGCAGTGAGGCAGTTGCCAG |
| | | CCTCTCTCAGCTGCCTCATGGGATTCGCACTGCAGCTGCGGCCCTGGCGCGACAAGGGCTGGACTTGGCCAGCGGGACGGTCCCTCACG |
| | | GCGCTGAGGCCCACACTCTGCGTGGAGCCTCCCCGTGCCCAGGCTACCCTGCAAGGTCCTCGGAGAGGCTTCCTCCAGCCCCAGCCCCC |
| | | ACACAGCTCCGGCCCAGGCCCGCTCTTCCCCATCCCAGTTGCTTTGCGCTGTATACGGCCAGGTGACCCCGAGCCGGCCCTGAGCCCTC |
| | | GTCCCGGCTTCCTCCCCTGTAAGCTGGGTGAAGGACTCCATGGCACCCACCTGAGAGGGTTGTGGCGAGGCCCAGGCCCCTCGTGCCCA |
| | | CACGGCCGGCGGCCCATGCCTGGCAGGGGCTGGGAGGAGGCTGGGGCGACCAGAGGGGAGCGGCCTGTCCTGGAGGAGGCCCAGGGA |
| | | CCCTGGTGAGAGGGTCTCTCCCAAGTGCTCTCTATGGGACCCCCTTCCTCTGCGCCCGTCCTTCACGGACCTCTCCGGGTCACCCCTGGG |
| | | CTGCACACTGGGTTCAGGGGGGCCTTGAGGTGGGGCCCCTGTTCCCAAGTCCCGGCGGGGTTTCTCCTGAACCTCAACCCATCCTCACCT |
| | | GCGGGCATTCCCATCCCCCAACGCCTGGGTCACCAGGATTCCAGGCAGGAGGGGCGGTGGGGGTTACCAAGGCCCGGGTTGCCATGCA |
| | | GAACCCCCAGCCACCACGCAGACCCCCACGGGGCCCAGGGAAGCTCCTGGTCTCACACTGCACCTCACACTTCCTGTGGGGGCAGACTC |
| | | CAAGGTCCCGGCCTCTCATCTTGTAGAAACTGAGGCACAGGAGGGACACACACTCCCACGGCCGGTCACCGTGGCCCCCACACCTCCCAC |
| | | TGGACTGACACCTGGCCAGGCTCCGGACACCCGTGGCACAGCCTCAGCCCCTGCGGCCCCTGCTCCGTGGCCCCCAGGCCCCAGCTCCC |
| | | ATGTGCACGTCCTGCCTCAGGCCTGGAGGCCCCTCGGCCCCAAATAATCAGACAATTCAACAGCAAAACTACTTTTTTCAGGCTGGCAGG |
| | | ACTCTGGGCAACCCCCTGCAACAGCCCCCTGCCCTATCACAGCCACCCTTGCCTCCCAGGCACGGAGACCCCACCATCAGGTCCCAGCCT |
| | | TGGTTCATCCCCAAGCACCCTGTGTGTTGGGATGGCGATGCTGGCTGAGCCCCTGCATCC |
| 253 | chr21: | AGGGCGTTTGGGAACACCCCTCCCGGAGGGGTGAGGCGGCCCAGCCTGCGGCTGCCAGAGGACACAGGTTCTGCTGCGGAACCTGCAG |
| | 46280500- | ACATGGCCATAACAGGCCACAGTGCTCGGGCCCACACAGCCTGGACCCACATGGCCCTGTGTCACCTCCTCAGGGGCAGGCTTCAGGGC |
| | 46283000 | CTCGACCCTAGAGGCTGCCCCTCGGTTCTGCTCCATGGACGGCGCAGGCAGGCCCAGGCCTGTGACGAGTTCACGGAAGCTCCAGGATG |
| | | ACCCCCGCTCTGCGCCCTCCTCCAGCATTCCAGACCACAAACCACTCTGGGCTAAAACGAGGCATCGCCAGAGCATCCCACTTCCTCGGA |
| | | AAGCTGCGGTCTGGGGACGCGTCTTGGCCCTGAAGAGGCTCCAGATGGCTCCCATCAGGCCTCTCCGCCTACGTGCGGCCGACATGGAG |
| | | TGACAGAGCGTCGGGGACACAGAATTCAGAGCTGGGCCTGGGGCTGCTTTGAGATACTGATGGCTGCCAGGGGGCACAGAGACCCGTCC |
| | | TGCAGACAGGGCTGTGAGGGCCACAGGGGGCCTCGGGGAGAGGCAGTGGGAGGGAGGACAGTGGGGGCCTCCAGCTGGGTGAGCAGC |
| | | TGGAGCGAGGGGGGCCCGGGGCTTGTGATGGTGCTGCCGACCCTAGAGGTGCCGGCCCCACGATGGAGAGCACGTAGTGCCCCCCGGG |
| | | AGTCAGGAGGCCGGGCCTGACCTCGGGGGCTGCAGCCAGGGGAGGCCGGCACCCCAGATAACCCCCAAAGAACTGCAGGCCCTGAGGC |
| | | GAGGCCAGAGTGGGGGCGGGGGCAGGTCCCAGCCGAGGAGGTGCTCCGTGCTGCCTCAGCAGAACCCATGATGGGCTGGCCCAAGGCT |
| | | CTGAAGGTGGAAAGGCCTCACACATTCTGCCCCGGCTGACGCCTTCCTTGGGCCAGTGCTCGGGGGTGTGTAACAAACGCCAAGACGCAT |
| | | TGTAAAGAAGGAAGCCTGCGTTTCCATCACCGGCTTAATATCAAACAAAAGTGCAATTTTGAAAATGTAGTCCAAGGTTTTCTGTGGTGC |
| | | GGAAATGGCCAGGCCAGACCTCCGTGGGTGGTCCTTCGTGTCCACGTCAGCGCCCTACATCCACACTGTGGGCACCATGACCTCACATGC |
| | | GGAGCGGAGCAGGGCCGGCGCCCGGAGAGCCAGGCTGGTCACGAACGAGGCCTAGAGGGCGTCAGGCCCCAAAGCACTCACAGGCTTCTC |
| | | CTCTGTCCTCGGGGCCTTCAGACACCTGCATGCGCCGATTCAGCCACCCGCGCGCGCCGATTCCCCTGGCCATGGGGTTTCCAAAGTGTG |
| | | TGCTCAGAGGACAGTTTCCTCCAGGATGACCTGTCAGTGGCTCTCTGTGCCGGGGACGTCGCGTGCTGGGTCCCGGTCTGAATGCTTCCT |
| | | AACGATTTACCCAGTTCCTTTTCTCCACTCAGGAGGCGTTTGCTGAGAGGCACAGGCTGAGCCCCCGTGCTGATGCCACGACCGAGGGAA |
| | | CGGGTCTCCCTGTCGGCGTGAACTGACCCGGCCAGGCGTCCACTGCCACTCGGACTGTCTCCCAGGCACGTGGCGCCCACACGGGCAGA |
| | | ACACGCCCTCCACACACGCGGCTTCGGGCAGAACACGAGGCGCCCTCCACACACGCGGCTTCGGGGCTTGTCATGAAAAAAGCTGAATG |
| | | CTGGGGGTGCAGCTTTCACCAACAGAATCCCGTTTGGAAGGGACGCGGTGAGACATGATCCACCCTAAGTTGTGATCCTGGGTGAGCCGC |
| | | CGTCCACACCCTGCTGAGGGTCCCTTCACCCACTTTATTCTCCAGAAAACCCTGCCCATCAGGGCTGAGTCCCACGCCTTCCCTCTCCGT |
| | | CCAGGCCTGGCTTTGACCTCTGGGGTCGTGTGGGGCACAGGGGACACCCTATCCAGGCAGAGGCCCTACGGCTATCTGGAGGAAGTGGTG |
| | | GGAGCTGGGCTTCTGCCTGGAGGATGCACCCAGAGGGGTCACAGTCCACACAGAGACACACGGGTGCCTTCCAGATGGCTGAGCCAGTC |
| | | CAGCCCAGAAGGGCCTGGGGGTTGGGGGCTGCACCTGGCCTGTCCCCACCAGCAGGGCTCAGGGCTTCCCAAGGTGTGTGGGGGACGGG |
| | | GCAGCACCTCTCAACCAGGTCACCTGAAACCCGAACTGAAAGGCATCCTAAGTTAAGACATTAACTCCCATTGTCAAGGTGCCATCGTCA |
| | | ATTCTGTCTCCAAATCCTTCTTTGTTATTTCATGTATTCACAGAGTGACGCTCCGTGTTTCGTTCAGCCTGCAGGCCTGCAGAAGCTGCA |
| | | TCTCGGGATGGCCAAGAGCCCGGCCAGGCCCCACGGCTGCACCCAGGACGGGATTCATGCCCCATGCCTGGCTTCTCACGACCACAGAGT |
| | | GCCTTTCCCGGGACTGGATGGAGGCAGAGTGAGAGAAGAGCCTGGAGCAAGTGTTTTGGACCACAGTGATCAAACACGGAGCCCGTGGG |
| 254 | COL6A2 | AAGAAAGGCCAGACCGGGCACGGTGGCTCACGCCTGTAATCCCAACACTTGGGGAGGCCGAGGCGGGCAGATCACCTGAGGTCAGGAGTT |
| | | CGAGACCAGCCTGGCCAACAGGGTGAAACCCCGTCTCTACTAAAAATACAAAAAAAAATTAGCCGGGCGTGGTGGCAGGCACCTGTAATC |
| | | CCAGCTAATCGGGAGGCTGAGGCAGGAGAAAATCACTTGAACCTGGGAGGCGGAGGCTGCAGTGAGCTGAGATCGCGCCACTGCACTCCA |
| | | GCCTGGGTGAGGGAGCGAGACTGTCTCAAAAAAAAAAAAAAAAAAAAAAAAAAAGGAAAGAAAGGCCCGGTGAGATGCTTTCTCTTAAAC |
| | | ACGGCCCTGCACGTTGAGTTGCTGCCTCCTGTGGCCTATTTCACGTTTATGCAAAGTCGGGCGCCTGATGCGGGGCTCACCCGCCACAAG |
| | | CAGGGGTCCTGGTGCTGCTCATGGAAGGGGCCCTACCCAGCCCGCGGGGCACTGGCTGGGACGGGGCTGCCCAGGTCCGCCCAGGATC |
| | | CAAACACCCAGCCCCGCCCAGCGGCCCTTCCTGGCCTGCAGTGGAGGCTGTAATGGGCAGGGGTGGTGGGAATCCCAGCTCACAGGGC |
| | | GCCTGCTCTTAGAAGGGCGGCATCTGGGTCCAGAGGTCAGAAACGTCAGATGCCCATCCCAGAAGTGGCGGGGA |
| 255 | COL6A2 | GGGTGAATGAGTAGATGTATGGGTGAGTAGGTGGGTAGGTGGGTAGATGGATGGGTGGGTGGGCGAGTGTGTGGTTAGATGATGGATGG |
| | | CTGAATGGATGAGTGGGGGGATGGATGGGTGAGTGGGTGTATGTATGGATGGGTTAGTGGGTGGGTGGATGAATGGATGGGTGCATAAA |
| | | GGATGGATGGATGAATGAGTTAGTGGGTTGGCAGATGGATGGATGGGTGAGTCAGTGGATAGATGGATGGGTGGGTGGATAGAGGATGG |
| | | ATGGTTGGGTAGGTGATGGGTGGATGAGTGGATAGATGGGTATGTGAGTGAGTGGGGGGATGGGTAGGTGGGTGGATGGATGGTTAGGT |
| | | GAATGAGTGGATGGACAGACGGACAGTGGGTGGATGGATGAGTGAACGGATGGACCGATGGATGAATGGGTGGGTGGGTAGAGGATGGA |
| | | CGGACAGGTGAGTGGGTGGGTGGATGGATAGATGGGTAAGTGAGTGGATAGATAGATGGGTGGGTGGACAGAGGATGGGTGGATGAATG |
| | | GATGGGTTAGTGGGTGGCTGGGTGGATGGATGATGGATGGGTGACTGGGTGGATGGATGGATGGGTTAGTGGGTGGCTGGGTGGATAGA |
| | | TGGATGGGTGATTGGGCGAATGGGCGAATGGGTGGATGGGTGGGCGTGGAGTTGGTGGGTACATGATAATGGGGTGGAATACCCATGGA |
| | | TTGGAATGAGCTGTTTTGGCTGCTATTTCTGGGACACCCAGCTCTGCCAGGCCCCTACCCCTCTGGTGGGCCAGGCTCTGACGGTGGCCA |
| | | CTCATGGCCTTTCTAGCTCTGGTGCCAGCATAGGGAAGGAGGAGGCACAGCCTTGTCTTACTCCTTGCACCTGTTAGCCCCCCCCCCCGC |
| | | CAAGGGAGGACCCGTGGTTGGGGACAGCACAGGGGGCCCTGCTGTGTGCAGGGACTGTCCCTGGGGCCACTGAAGCCCACCTGTTCTTG |
| | | TTCCTTCTCAGGCGGATCCTGGTCCCCCTGGTGAGCCAGGCCCTCGGGGGCCAAGAGGAGTCCCAGGACCCGAGGTAGGTTGGTGGCCA |
| | | GTCCCCATGCCCTCCCCCCAACCTGCCAGGCCAACACACACCCAAGCCTCGTGGTTCTGCCCACGGTGGACCCACGTATCAGTGGGCAGT |
| | | GGCCTGGGAGAGACTCAGCCACCCAGCCTTGGCCCCAGAGTCTCAGCCTCATCCTTCCTTCCCCAGGGTGAGCCCGGCCCCCCTGGAGA |
| | | CCCCGGTCTCACGGTAGGTGTCACATGGGGCAGAACCAGTGTCCTTCTCCTGCCAAAACTAGACACCAAGAGCAGCAGGGGTGGGGGAA |
| | | GGTCAGCTGGCACGGTCAGAGAGCAAGATCAGTGGAGGAGGTCAGAGGGCAAGGTCAGAGAGCAAGCTTGGTTGGGGAAGGTCACAGG |
| | | GCAAGGTTGGTGGGGGGAGGAGGGTGGCAGCGAGGTTGGTAGGGACAGGACCCGCCAGCCTCCCCGCATGGCTGCCTCCACACGTGGG |
| | | CTGGAATGTCCCGGGACCCCCAGGCCAGGACCTTGCTGTGGAAACTCTTCTGGGGCCCCGGGGGGACTACCCTGCCTGCCGTGTGCATT |
| | | GCAGGAGTGTGACGTCATGACCTACGTGAGGGAGACCTGCGGGTGCTGCGGTGAGGCACTGCCCACGGCAGGGTCGGGGCCCATGCAC |
| | | CGGGTGGAGGGCGGGAGTGCAGCAGGGCTGGGTCATCGCTGGGTCCTGCATGTGCACGTGACCCTAGGGTCTGAGGTCTCCCCGGTAC |
| | | CCCCCGATGACCCTGCCACCCCCCCAGACTGTGAGAAGCGCTGTGGCGCCCTGGACGTGGTCTTCGTCATCGACAGCTCCGAGAGCATT |
| | | GGGTACACCAACTTCACACTGGAGAAGAACTTCGTCATCAACGTGGTCAACAGGCTGGGTGCCATCGCTAAGGACCCCAAGTCCGAGACA |
| | | GGTCAGCGGGGCAGGGGCGGGTGCAGCATTGCGGGGGGCCGGGCGGGGCGTGGGAGGCGATGAGATGGGAGAAGTCCAGACGCGTCC |
| | | CTCCAACGAGGGCCTCTGCATGGCTGGGGATGCCCCAGACCCCGAGGCCTCTGGCAACGACCTCACGCGTGCGGCTTGCAGGGACGCGT |
| | | GTGGGCGTGGTGCAGTACAGCCACGAGGGCACCTTTGAGGCCATCCAGCTGGACGACGAACGTATCGACTCCCTGTCGAGCTTCAAGGA |
| | | GGCTGTCAAGAACCTCGAGTGGATTGCGGGCGGCACCTGGACACCCTCAGCCCTCAAGTTTGCCTACGACCGCCTCATCAAGGAGAGCCG |
| | | GCGCCAGAAGACACGTGTGTTTGCGGTGGTCATCACGGACGGGCGCCACGACCCTCGGGACGATGACCTCAACTTGCGGGCGCTGTGCG |
| | | ACCGCGACGTCACAGTGACGGCCATCGGCATCGGGGACATGTTCCACGAGAAGCACGAGAGTGAAAACCTCTACTCCATCGCCTGCGACA |
| | | AGCCACAGCAGGTGCGCAACATGACGCTGTTCTCCGACCTGGTCGCTGAGAAGTTCATCGATGACATGGAGGACGTCCTCTGCCCGGGTG |
| | | AGCGTGTGGGCGCGGGGCAGTCGGCCGAGGAGCAGCAGGCCCCAGCCGCTGTCTAGCGTGAGCCCCAGGGACACCCCTCACCTGAGGG |
| | | ATGAATGTGCAGCCCAGGATCTTGGGCTGTGGGTGGGAAGGGGTCGGGCCCTCTCGGGGCTGCAGGGCAGAGGCCAGCTGCACCCTGA |
| | | GCCTGTCTAGGCAGATCAGTGAACGGCCGCTGAGGGTTCGCTAGGGACTGACCCTGGCCTGGCCCGGCCTCTCTCCTCTCTTCCAGACCC |
| | | TCAGATCGTGTGCCCAGACCTTCCCTGCCAAACAGGTAATGCAGGGCACCCTGAGCCACCACCCCAGACTAGCAAAGCAGCCCTGGTGTC |
| | | CTTCCTCCTCGAGGGCCGGGCTGGGGGAGGGGCCGTGCAGGGACCCGGGGGGCGGCGGAGCCACTGCGGAGGCTGCTCCTTAGGGAG |
| | | ATGGCCCCAGGATGGCAGCACAGGGGAGGAGGGGCTTGGGGAAGGCAGGCTCCCAGGAACGCAGGAACAGCATCACGAGGCCATGAGG |
| | | TGGGTGCTGCTAGCCTGGCGCTGTGCTCGGCATGTGGCCACTGGTCTTGAAGGCCCACCATGGGCCTTGCAGTCTCCCTCAGCTGCCGC |
| | | CCAGCTCCCATGGGCTGGCCGTGCATGTGCCACTCGGAGGAAGCCCTGGATTCAGTGAGTGAAACCATCCCGGGGTGGAAGCACTGACA |
| | | CCCCCCAGCACCAGCAGGTCTTGCTCCAACCCTGGCCTGCCTCGGAGCTGCAGCTGCGGCTCTCACATCTCTGGGAGTGGGGGAGCCCA |
| | | TGTCCCGGATGTGGCCCACGTGGGTGTGAAGCTGGAGCTGGGGGTGCCGTCCAGGCTCTGCTGGACGTGGTGCTGCCCCCATGGTGCAC |
| | | TGCTGCACCGTACCTGGGCCCACAGGAGGTCCCCGGGGGCGTTAGGAGCTGAGTCCCCCTCAGTGAGCCGTCCCCTCCAGGAGTGTGAG |
| | | GGTAGGGATGCCATGGAGACAGGGTGGGAGGGTCCGACCTGGAGGACCACAGGGAGGAAACCTCAGGGTCTGCGGTACGAAGTCAGCG |
| | | CTTCCTCAGCACGCGGGTCGCGGTGTGCGTTCGGGCGTTCCATGGGGAGCTCCCGGTGGGTGAGCTGGGCCACTGAGCACATTCACAGG |
| | | CCCTGAGGCTGCCCCAGGGGAGGAGCCGTGGACTCAGAGCCGAGGTTCCCCATACGTGCTGCGACAGAGAACCTAGGGCTTGCACCTGG |
| | | GTCTGGCTGCCCTTCAGCAGGCGGGCAGCCTCTGGCCCCACAACAGTGGGCTGTGCTTCTGCCGCCAAGGTGCAGGCGTCCTCCCCCAG |
| | | GGTCCACATCAGCAGCAGGGGCACCTGGACCCTGAGGGCAGGAACCAGACCTTGGCTCCTCCACCCACCCCCTCGTTCCTGATGGGGCA |
| | | GGGAAGTCTCGGGACCCCATGATGGGCGACATGGCGATGGTCACTGTGGGTGCTTTGCTATCAGGTGGGGGGCCTTCCTCTCCACTCTGG |
| | | GTCCAGTGTGAGTGGCCGCTATGGCTTCCCCTCCACTCCAGGTTCTATCGTGAGTGGGTGGGTGCTGCGTCTGTGGATGTCACGTGACCT |
| | | TTCCTCTTTAGCCTATCATTGTAGTTGGGAGTTAGTTAGCCCGTTGAGCGTCATTGAATTTCCAGTGTTGAGCCAGCCCTGCGTGCCCGG |
| | | GATAAACCCACCTGGCCGTGGTGTGTGGCCCTGTTTATGCACGTGGGCCCTGATTCGCTGATGCCTGCCTGAGGGTTTGCGCTTATCGGC |
| | | GACATCAGCCTGCACTTTTCTTTTCTCGTGATCTCTCTGGTTCTGGCCTCAGGGTGACGTGGGCCTCGTAGGGTCCTGTGGTGGCTCCTC |
| | | CCCAGACGGTGACATGGAGTGAGCCCATTCTCCCTCCTGGGAGTGGGTCACTCAGGCCACCAGAGCACCACAGGGAAAGCAGCCAGGGAG |
| | | GACACGGAGGCCCTTGAAGCTCTGGCCTCTTCTGAGGCCTCCAGGACCTGACAGTGAGTGGGAGCAGCCCTGGCAGAACCCCTCCCCTCC |
| | | TCTCGGCCGCCCTGACACCTCATCCCCGACACTCAGAGCTCATCCTCCTTCCCAGCTGTTTCCAATTTCAAAGTGAACTCGACCTTGTGG |
| | | CTCCAGGAGATGCAGCAGGGACAGTGTTAAATCGGCTTTCACCAGCCCACACGGCCAGGCATCCTCCTCGGCCCTCCTGGGCACTGGGTG |
| | | GACACCACTGGCTGTGGCCTGGCCCTGGCCTTCTCCAGACAGCCCTGTCCACCCCAAAGCCCAGCCACCCTGGGCCTGCAGCAGGCCTGT |
| | | GGAGTTCTCAGTTGCGTGGGGACCAGAGGGTGCTGGAGAAACAAACCAGACGCAGCTGAAGGCAGTCAGGGCAGGGCGCAATCAGCGAT |
| | | AAGAGCTGCATAGGGGCCACAGCGTAACCTGAGCTCCAGTCGGTGGAAAGAAAAGGCAGAGACGTTGCAGAGGCCAGGTCTGCTCAGGG |
| | | GAAGACAGTTCTGGGTGTAGAGGACTCACATCCCAGAGAGGCTGAGGAAGGGTTTACCACCGCAAGCTTTCTCAGGCGGGCTCTTGAGGG |
| | | GTGGCTGGGGTCTTCCTGGCGACGGGCCTGCGGCACTGGAAGCCCTACTGGAGTTTGGCCTGTCTCCGGCACAGGTTTGGACGGAGCTGT |
| | | TTTGTGCTGAAAGGTTTTCTCGGGGTCCGTGGTGTCCCCCAAAGGTGCCACCGTGCGGGTCTCCTAGCTCCCTGCCAGCTTCCTGTCCCT |
| | | GTGCTCACTGCCCCCACGCCTCCTGCCAAGGCCGAGCCACACACCCGCTCCACCTGCATTTCCTCTACCGACTCGCCAGCCCAAATGCCG |
| | | CTCTTCACTCTGGCCTCGCTGAGCGGCTGCCCGAGGAGGAGCTCTAGGCCGACGCCCACCGCAGGCCTTACAGTCTTCTCTGGACGCTCC |
| | | CTTGCAGATGCACCGTGGCCTGGCGGCGAGCCCCCGGTCACCTTCCTCCGCACGGAAGAGGGGCCGGACGCCACCTTCCCCAGGACCAT |
| | | TCCCCTGATCCAACAGTTGCTAAACGCCACGGAGCTCACGCAGGACCCGGCCGCCTACTCCCAGCTGGTGGCCGTGCTGGTCTACACCGC |
| | | CGAGCGGGCCAAGTTCGCCACCGGGGTAGAGCGGCAGGACTGGATGGAGCTGTTCATTGACACCTTTAAGCTGGTGCACAGGGACATCG |
| | | TGGGGGACCCCGAGACCGCGCTGGCCCTCTGCTAAAGCCCGGGCACCCGCCCAGCCGGGCTGGGCCCTCCCTGCCACACTAGCTTCCC |
| | | AGGGCTGCCCCCGACAGGCTGGCTCTCAGTGGAGGCCAGAGATCTGGAATCGGGGTCAGCGGGGCTACAGTCCTTCCAGGGGCTCTGG |
| | | GGCAGCTCCCAGCCTCTTCCCATGCTGGTGGCCACCGTGTCCCTTGCTGCGGCTGCATCTTCCAGTCTCTCCTCCGTCTTCCTGTGGCCG |
| | | CTCTCTTTATAAGAACCCTGGTCATTGAATTTAAGGCCCACCCCAAGTCCAGAATGACCTCGCAAGACCCTTAACTCACTCCCGTCTGCA |
| | | GAGTCCTTCTTTGCTGCATCAGGTCACCCTCACAGGCTCCAGGGTTTGGGTGTGGAAGTCTTTGGAGGCCCTTACTTAGCGGCCCAGCTG |
| | | GGCTGCCGTGCGTCTGGGATGGGGCTGAGGGAGGGTGCTGCCCAGGTGCTGGAGGATGTTCCAGCACCAGGTTCCAGCGGAGCCTCGGAA |
| | | ACAGGCCCCAGAGGCTGGTGAGCCTCGCTGGGTGTGGGCACTAATCCCGTGCATGGTGACTCGTGGGCGCTCACGGCCCACCTGGTGGC |
| | | AGGTGAAGGCTTCCGGTTGGGCAGCAGATAGTCCTGGGGGAAGCTGGCAGTCCTGGCACCATGACGTATCTGGGCTGGTGTCATGCACA |
| | | GTAGGGCGAATGGCCACAGCTGCCTGCCAGCAGCCCTGATCCCGGGGTGTCTGCACCCTTCCAGCCCAACCTCTGGGTCTCCAAAAGCAC |
| | | AGTCGGGGGAGCATCCACCAGGCACAACCTCTGCGGTCCTCAGAGGACTGAGCAGAGAATCCCAGGGTCCACAATGTTGGGGAGCGGCA |
| | | GGGATCACCATCCAAAGGGAGCGGCCCCCACGGCGAGCTGACCCCGACGTTCTGACTGCAGGAGCCCTCATCCAGGCTGGGCTCCTGCC |
| | | GGGCACGGCTGTGACCATTTCTCAGGGCCAGGTTCTCGTCCCCACACCCACTGCACAGGGCAGGCCAGGCTGGTCTTCCCACTGTGGGG |
| | | ATGAAGGATCCTCCACAGGAGGAGGAGAGCAGAGTCCACAGACATCCCAACAGCCTCAGCCTCCCTGTGCCTGGCCGGCCCCCACAGCTT |
| | | CCCCGTCTCCTCCAGGCCCCACAGACACTGATGAATGGACAGAGACCCCCAAAACCAGCTGCCCCTTGCATGTCTGTCTCCATATGTTTG |
| | | GTGACAGCAGTGAAAATGTTATTAGTTTTGAGGGGGTTTGGGAAGCCCAGCGGTACCTGAGGAGTTTCTGGACATTTAAGCCGGTTCCTA |
| | | GGTGTGGCCTTAACAGGGAGGCTGCCCTTCCTTTCACTGAATGAGCTGCGTCACTCATAAGCTCACTGAGGGAACCCCATCTGCCAGCTC |
| | | GTGCGTGCTCAGACGGCGTCCATGTCTCAAGCGTTCTGTGAAGGCTGCGGTGCAGCGTGAGGTCACCCTGCTGTGTTCAGAGCTTTGCTC |
| | | ACTGCCTGCGGGGCTGGACCGTTGCACCTCCAGGGCCCCCAGAAACCGAGTTTCGGGTCAGGGTCCTCTGTGTGCATTCCTGGGGGTCCA |
| | | TGTACCAGCTGTGACGACGTCCAGGGGTTGGGCTGAGAAGCAGACACCCTTGGGGAAACTGGCTCTGTCCCTCCCCTCCCCCATCCCAGG |
| | | AGCTGAGGTCTTGGTGAGGCCACAGGGCCAGGTCCACGCAAGGACTGTCCGTGTCCTGTCCTGTGGTCTCTGGCCCCACGTGACACCCAC |
| | | ACGTGTGGTAGGCAGCCTGGCCTGGGTTGTGGCTATGGCCAGGCCCCCAAGCTGTCCCCGATGCCCAGGGCTGGTGACCACCCAGGCAGG |
| | | TGGGGGCCCCACTTGGTAACAGAGTCATAGGGCAGAACCCACCTGGGCTGCCACAGAAGGTCTGGCTGCCCCTGTGCCCACTGCTCCCCA |
| | | CCATGGCCAATCAGAAGAGTCAGGGGCTCCTGGTCTTTCCGGGAGGGACGTGGCCCAGCCAGCTCTAGGTGTTCTGAGCAGCTCTGGGA |
| | | CCCAGCGATTGAGGGGTCAGGCTGGGGGTGTCAGAGCCAGGGTCCTCCTTAAGTACCTCCCACACTACACAGACAGTGGCCCTTTTGTGG |
| | | GCAGCAAATTCTTGAGCCATGAAAGGATGCTTTGGGCCCCTTCCCTCCCAGGAGGGCAGCCTGTGCAGGGATGGTGCTCAGCAGGTGGAC |
| | | AGGGCCTGGGGCCTGTGTCAGGGTCTCAGGCCTGGGAGCACCAGCAGAGGAGATGGCGGCTCCCAGCAGTGCCGCCTGAAAGTGTCTTG |
| | | GGCTAAGGACCCACACCCAGGGCTGCCCTGCAGAAACGCCCCCGCAGAGCCCAGTGGTCTGTGAGGTTGCAGGCAGGGTGCGAATGGAA |
| | | GGGCACAGGTGCGGGGCTGGCACCTGCCCGGTCCTGCCCACCTCCCCTCCGCCCAGCCCGCACCTGCGTCTCCCCACAGAGCTGTCCGT |
| | | GGCACAGTGCACGCAGCGGCCCGTGGACATCGTCTTCCTGCTGGACGGCTCCGAGCGGCTGGGTGAGCAGAACTTCCACAAGGCCCGGC |
| | | GCTTCGTGGAGCAGGTGGCGCGGCGGCTGACGCTGGCCCGGAGGGACGACGACCCTCTCAACGCACGCGTGGCGCTGCTGCAGTTTGG |
| | | TGGCCCCGGCGAGCAGCAGGTGGCCTTCCCGCTGAGCCACAACCTCACGGCCATCCACGAGGCGCTGGAGACCACACAATACCTGAACT |
| | | CCTTCTCGCACGTGGGCGCAGGCGTGGTGCACGCCATCAATGCCATCGTGCGCAGCCCGCGTGGCGGGGCCCGGAGGCACGCAGAGCT |
| | | GTCCTTCGTGTTCCTCACGGACGGCGTCACGGGCAACGACAGTCTGCACGAGTCGGCGCACTCCATGCGCAAGCAGAACGTGGTACCCA |
| | | CCGTGCTGGCCTTGGGCAGCGACGTGGACATGGACGTGCTCACCACGCTCAGCCTGGGTGACCGCGCCGCCGTGTTCCACGAGAAGGAC |
| | | TATGACAGCCTGGCGCAACCCGGCTTCTTCGACCGCTTCATCCGCTGGATCTGCTAGCGCCGCCGCCCGGGCCCCGCAGTCGAGGGTCG |
| | | TGAGCCCACCCCGTCCATGGTGCTAAGCGGGCCCGGGTCCCACACGGCCAGCACCGCTGCTCACTCGGACGACGCCCTGGGCCTGCAC |
| | | CTCTCCAGCTCCTCCCACGGGGTCCCCGTAGCCCCGGCCCCCGCCCAGCCCCAGGTCTCCCCAGGCCCTCCGCAGGCTGCCCGGCCTC |
| | | CCTCCCCCTGCAGCCATCCCAAGGCTCCTGACCTACCTGGCCCCTGAGCTCTGGAGCAAGCCCTGACCCAATAAAGGCTTTGAACCCATT |
| | | GCGTGCCTGCTTGCGAGCTTCTGTGCGCAGGAGAGACCTCAAAGGTGTCTTGTGGCCAGGAGGGAAACACTGCAGCTGTCGCTCGCCCA |
| | | CCAGGGTCAATGGCTCCCCCGGGCCCAGCCCTGACCTCCTAGGACATCAACTGCAGGTGCTGGCTGACCCCGCCTGTGCAGACCCCACA |
| | | GCCTTGATCAGCAAACTCTCCCTCCAGCCCCAGCCAGGCCCAAAGTGCTCTAAGAAGTGTCACCATGGCTGAGGGTCTTCTGTGGGTGGA |
| | | CGCATGATTAACACTAGACGGGGAGACAGCAGGTGCTGAGCCTGTTGTGTTCTGTGTGGAGATCTCAGTGAGTTTTTGCTGTTCAGACCC |
| | | CAGGGTCCTTCAGGCTCAGCTCAGGAGCCCCACAGTGAACCAGAGGCTCCACAGGCAGGTGCTGACCTGACAGGAGTGGGCTTGGTGGCC |
| | | ATCACAGGGCACCACAGACACAGCTTGAACAACTACCAGTATCGGCCACAGGCCTGGAGGCATCAGCCGGGCCATGCTTCCTCTGGAGGG |
| | | CTAGAGGAGGACTAGAGAAGGGCCTGCCCCGGCCTCTCCCCAGCATCCCAGGGTTCCTGATCTCCTGGATAAGGATACAAGTCACCACAC |
| | | TGGACTGGGGCTCAGCCTGCTCTAGAATACCTCACCTAAGTCACAGTGGACCAGGCTCAGCCTGCTCTAAGGTGAGCTTACCCGAGACAC |
| | | TGGACCAGAGATCAGCCTATCCTGGGATAAGCTCACCCGAGTCACACTGGACCAGGGCTCAGCCTATTCCGGGATGAGCTCACCCGAGTC |
| 256 | C21orf56 | GACACTTCCATGACTGCAGCTGACCAGTCCACCTGCCAGCGGTTGACCACTCCCACTTCGCCAGCGACCGAAGGGGAGGGGAGGGGCCT |
| | | CACCTGAGGGCAACAGCAGAACCCACCACCTGGTCTTGCTTTACTCAGACCTGAGGGTGTGAAAGGTGCCCGTGACCTCCCGCATCAGGG |
| | | AGCTGGCCGCCACCCTCGACTCCCGGGGAGCAGGCGTCCCGCGACCCCCTCATCTACCAGGCCATCTGAGCTGGGCGGCGCCTCACCTC |
| | | CGCTCCCGGGGGAGCCGGCCTCAGGGTAGGCATGCGCCCTGGGTGGGAGCAGGTCGTGGCCGCCGCCCTCCTGGCAGCTCTGGCTGAG |
| | | CAGCCGCCGCAGCATCTGATTCTCCTTCAGGAGGCGCACCTGCTTCTTCAGGTCCGCGTTCTCGCTCAGGAGCCGGCTCATCAGCTCGCC |
| | | GCCTTCAGCCATGGCGGGTGCGTCCCTCCTTGTCCCTCACGGCTCCTGCAGCCCCATGGAGGTGGGAGCCCAGAGCCCGCAGGCACCAC |
| | | AGAAACAGCCCAGGCACGGAGTTCCGTAGCCACCACCGCCTTCCACGCCTTGTGATGTCACTGCCCTAGTGATGAGGTGCCCAGCACCCT |
| | | GCCTGCCCCCGCGATGGCTCATGGCCCCGTTGAGGCAGTGAAGCTGGAGGCCCGTGGCGTGCACAGGCAGCCACTCCCACATTATGACC |
| | | AGGGCCCGAGAATGCCAAGGACATTAGGCAGCTACGGGATGTAGCGACTGTACTCCAAGAGGGGCGTCCAAGCCACTCCCCATTGA |
| 257 | C21orf57 | AGGTGGAGGTTGCAGTGAGCCCTCCTCCCCTCCTCCCCCTTCCCTTCCCACCTCCCATGCCCCCCTTTCTTCCTCCCACTCCCCTCCCGA |
| | | GGCCCCGCTTATTCTCCCGGCCTGTGGCGGTTCGTGCACTCGCTGAGCTCAGGTTCTGGTGAAGGTGCCCGGAGCCGGGTCCCGCCTTCG |
| | | GCCTGAGCTAGAGCCGCGCGGGCGGCCGGCTTCCCCCAAACCCTGTGGGAGGGGCATCCCGAGGAGGCGACCCCAGAGAGTGGGGCG |
| | | CGGACACCTTCCCTGGGGAGGGCCAG |
| 258 | C21orf57 | CCTTCCAGATGTTCCAGAAGGAGAAGGCGGTGCTGGACGAGCTGGGCCGACGCACGGGGACCCGGCTGCAGCCCCTGACCCGGGGCCT |
| | | CTTCGGAGGGAGCTGAGGGCCGCGTTCCTTCTGAAAGCGGGACGCGGGAGGGGTGGAGGCTGCGGGGAGCCGGGGTCGCACACGAATAAA |
| | | TAACGAATGAACGTACGAGGGGAACCTCCTCTTATTTCCTTCACGTTGCATCGGGTATTTTTCGTTATTGTAAATAAAACGGTTCCGAGC |
| | | CGTGGCATCGAGAGGGCGTCTGGAGTTCAGGGAACGCGTGGCCCCCGCCCGGGAGCACCGCGCAGCGCTCGCCTCTCGCCCTTCAAGGG |
| | | GGTCCCTGCCCGGAGCCTGCGCCCCCGGAGAGGAAGGGGCTCGAGGGGCTTGGGTGCCGCAGCGCGTCCTTCCGTAGAAAAGGCTTGC |
| | | GTCAGTATTTCCTGCTTTTACCTCCTGAG |
| 259 | C21orf57 | CAGTATTTCCTGCTTTTACCTCCTGAGTATTGGAATATTCGAGTAAACCCTGGAGTTTCAGCGCCAGCGCACGCCTCTTCATCAGGGCAG |
| | | CGCGTCGCGAGCGCGCTGGTTCCCCGGGGCCTCCCGGCCACGGACACCGCTCTAGCCAGGGCCACGGCGAGGCCGCCGAGCAGCACCTCA |
| | | GAGACCTGCGTGAGTTCTAAAGCCTGGGGCTACTACAATTCTGCTCATCTGTTTGTCCTGTGAAATGATTCAGGGACATGAAAATGCCTT |
| | | CCCACTGACTTGCGTCCTGTCTTAGCCTGGACTTGTCCCCTTGGGAACACGGGCCAGGCCCCTCTGTTCCTGAAGT |
| 260 | C21orf58 | ATGTCTGCAGGGAAGAAGCAGGGGGACCCTGAATAAAGTTTCCGTTTTTCCTATTTGTTAAAGTGATAGAGCATTATAGGACCAGAGAAC |
| | | AGGTGTGTCTGTACACTGTGCAGGTCCCCGGGGCAGGCTCTGAGTCCGTCTGCACACGGTGCGGGTCCCCGGGGCGCGCCCTGAGCCCGT |
| | | CTGCACACGGTGCGGGTCCCCGGGGCGCGCCCTGAGCCCGTCTGCACACGGTGCGGGTCCCCGGGGCGCGCCCTGAGCCCGTCTGCA |
| | | CACGGTGCGGGTCCCCGGGGCGCGCCCTGAGCCCGTCTGCACACGGTGCGGGTCCCCGGGGCGCGCCCTGAGCCCGTCTGCACACGG |
| | | TGCGGGTCCCCGGGGCGCGCCCTGAGCCCGTCTGTACACGGTGCGGGTCCCCGGGGCGCGCCCTGAGTCTCTACTAAAAATACAAAAAT |
| | | TAGCCAGGCGTGGTGGTTCAAGCCTGTAATCCCAGCTCCTTGGGAGG |
| 261 | PRMT2 | CATACATGGTTATTAGAAAAGGCATCTCATCCAAATGTGGTGGCTCGTGCTTGTAATCCCAGTGCTTCAGGAGGCCAAGGGAGGAGGATT |
| | | ACTTGAGCCTAAGAGTTTGAGACCAGCCTGGGCAACACAACAAGACCTTGCCTCTACAAAAAACTTAAAAACTAGCTGGGTATGATGGTG |
| | | CACACCTGTAGTCCCAGCTACTTGGGAGGCGGAGGCGGGCAGATCGCCTGAGGTCAGGAGTTCGAGACCAGCCTGGCCAACATGATGAAA |
| | | CCCCGTCTCTACTAAAAATACAAAAATTAGCCGAGTGTGGTGGTGCATGCCTGTAATCCCAGCTACTCAGGAGGCTGAGGCAGGAGAATC |
| | | ACTTGAACCCGGGAGGCGGAGGTTGCCATGAGCCGAGATCACGTCACTGCACTCCAGCCTGGGTGACAGAGCACAAAAGACAGGCATGAC |
| | | TTTGTACTTAACTGCTCAGCTTTGTAATCACTGGGGGCCCAGATGCTCACTTGGATTCTAACTTTGTTGGCATCTGGGCCTAAAAGCCGT |
| | | GATGCAGGTGAGCAATGATGCAGAGGGCTCTGTGCGCCTGGCGGGCTCTGTTTGCCTGCTGGGCTCTGTGCGCCTGCTGGGCTCTGTGCG |
| | | CCCGGGAAGGTGCGGCCACCCTCACGCGGAAGGCGGCCAGCGGATCCCGGTGCGCGCAGCTCCCAGCGCTGGGGTTCCAGCGCCCCGCCT |
| | | CTTCCTATAGCAACCAGCGGGACCTGCCGTCCCCCGGGGCACCCCGAGGGGTCTGCGCCCGCTTCTTTCCGAAACGGGAAGGCGCTGGGG |
| | | GCTCGGCAGCCAGAGGGACGGGTTCAGGGAGCGTCCGGTGAGCCTAAGACGCGCCTTTGCCGGGGTTGCCGGGTGTCTGCCTCTCACTT |
| | | AGGTATTAGGAACCGTGGCACAAATCTGTAGGTTTTCCTCTGGGGGTGGGCGGAGGCTCCAAACCGGACGGTTTTCTCCTGGAGGACTGT |
| | | GTTCAGACAGATACTGGTTTCCTTATCCGCAGGTGTGCGCGGCGCTCGCAAGTGGTCAGCATAACGCCGGGCGAATTCGGAAAGCCCGTG |
| | | CGTCCGTGGACGACCCACTTGGAAGGAGTTGGGAGAAGTCCTTGTTCCCACGCGCGGACGCTTCCCTCCGTGTGTCCTTCGAGCCACAAA |
| | | AAGCCCAGACCCTAACCCGCTCCTTTCTCCCGCCGCGTCCATGCAGAACTCCGCCGTTCCTGGGAGGGGAAGCCCGCGAGGCGTCGGGA |
| | | GAGGCACGTCCTCCGTGAGCAAAGAGCTCCTCCGAGCGCGCGGCGGGGACGCTGGGCCGACAGGGGACCGCGGGGGCAGGGCGGAGA |
| | | GGACCCGCCCTCGAGTCGGCCCAGCCCTAACACTCAGGACCGCCTCCAGCCGGAGGTCTGCGCCCTTCTGAGGACCCTGCCTGGGGGA |
| | | GCTTATTGCGGTTCTTTTGCAAATACCCGCTGCGCTTGGACGGAGGAAGCGCCCACGCGTCGACCCCGGAAACGAAGGCCTCCCTGATGG |
| | | GAACGCATGCGTCCAGGAGCCTTTATTTACTCTTAATTCTGCCCGATGCTTGTACGTGTGTGAAATGCTTCAGATGCTTTTGGGAGCGAG |
| | | GTGTTACATAAATCATGGAAATGCCTCCTGGTCTCACCACACCCAGGGTGACAGCTGAGATGCGGCTTCTCCAGGGTGGAGCCTCCTCGT |
| | | TTTCCAGAGCTGCTTGTTGAAGTCTTCCCAGGGCCCCTGACTTGCACTGGAAACTGCTCACCTTGGCATCGGGATGTGGAGCAAGAAATG |
| | | CTTTTGTTTTCATTCATCCTAGTGTTCATAAAATGGAAAACAAATAAGGACATACAAAAACATTAATAAAATAAATTAATGGAACTAGAT |
| | | TTTTCAGAAAGCACAACAAACACAAAATCCAAGTATTGCCATGTCAGCAACACATTCCTACTTTAAGTTTTATGAAGTTAATTGGAGTAG |
| | | TGGAGAACAAAAGTGGATGTGGGGCAG |

### Example 4: Fetal DNA Quantification Using Massively Parallel Shotgun Sequencing

In this example, fetal-specific DNA methylation markers were utilized to quantify the fraction of circulating cell-free fetal DNA in maternal plasma, using a massively parallel shotgun sequencing (MPSS) platform. For this Example, four types of DNA markers were assayed: 1) fetal-specific methylation markers which allowed selective enrichment and subsequent quantification of fetal DNA (e.g., SOX14, TBX), 2) Y-chromosome markers which confirmed fetal DNA quantification (for samples with a male fetus; e.g., SRY1, SRY2, UTY), 3) total markers avoid of restriction sites which were used to quantify total cell-free DNA, including fetal and maternal DNA (e.g., ALB, APOE, RNAseP, and 4) digestion control markers which monitored the completeness of restriction digestion and hence the accuracy of methylation marker-based fetal quantification (e.g., LDHA, POP5).

### Methylation-Specific Restriction Digestion

Fetal methylation DNA markers were enriched by selective digestion of unmethylated maternal DNA, using methylation-sensitive restriction enzymes. Digestion was performed according to the parameters specified in Table 5 below.

TABLE 5
Methylation-specific restriction digestion

| | Concentration | Reagent Volume |
|---|---|---|
| Reagent | in reaction | (μL) for n = 1 |
| H2O | N/A | 16.7 |
| 10 × PCR Buffer | 1 | 3.5 |
| (20 mM MgCl2, Roche) | | |
| 25 mM MgCl2 (Roche) | 2 | 2.8 |
| Exol [U/μl] (NEB) | 0.2857 | 0.5 |
| Hhal [U/μl] (NEB) | 0.2857 | 0.5 |
| Hpall [U/μl] (NEB) | 1.4285 | 1 |
| DNA [μl] | | 10 |
| Final Vol: | | 35 |

Reaction conditions:

| | |
|---|---|
| Digestion | 41° C. 60′ |
| Inactivation | 98° C. 10′ |

### Competitive PCR

The digested samples were amplified by PCR together with known copy numbers of competitor oligonucleotides. The competitors were synthetic oligonucleotides having the same nucleotide sequences as the target DNA, except for one base difference at the synthetic target site, which differentiated the target DNA from the competitor. Competitive PCR using target-specific primers allowed for independent quantification of each marker. Competitive PCR was performed according to the parameters specified in Table 6 below.

TABLE 6
PCR amplification

| | Concentration in | | Reagent Volume |
|---|---|---|---|
| Reagent | reaction | | (μL) for n = 1 |
| Water, HPLC grade | N/A | | 6.64 |
| 10 × PCR Buffer | 1 × (2 mM | | 1.5 |
| (20 mM MgCL2, Roche) | MgCl2) | | |
| 25 mM MgCl2 (Roche) | 2 | mM | 1.2 |
| dNTPs (25 mM, Roche) | 500 | μM | 1 |
| PCR primer (1 uM each) | 0.1 | μM | 5 |
| FASTSTART PCR Enzyme | 0.1 | U/μl | 1 |

| | |
|---|---|
| (5 U/μl, Roche) | |
| Competitor MIX | 0.38 |
| (8000/800 c/ul)(1:0.1 c/μl) | |
| DNA (from | 35 |
| restriction digestion) | |
| Total | 50 |

PCR Cycling conditions:

| | | |
|---|---|---|
| 95° C., | 5 min | |
| 95° C., | 45 sec | 35 cycles |
| 60° C., | 30 sec | |
| 72° C., | 45 sec | |
| 72° C., | 3 min | |
| 4° C. | hold | |

### Adaptor Oligonucleotide Ligation

Illumina adaptor oligonucleotides (TRUSEQ adaptors) were ligated to the amplicons generated in the competitive PCR described above. The adaptor-ligated amplicons were subsequently sequenced using the Illumina HISEQ 2000 platform (Illumina, San Diego CA). Two different ligation-based approaches were used to flank the amplicons with the adaptors. The ligation procedure was optimized to maximize the amount of double ligation products (i.e., adaptor oligonucleotides ligated to both ends of the amplicon), and minimize single ligation and/or empty ligation (i.e., two adaptor oligonucleotides ligate to each other without amplicon insertion).

### Direct Ligation of Adaptors

To render the PCR amplicons compatible for MPSS, the amplicons (which had 3′ adenine (A) overhangs generated by Taq polymerase during the PCR reaction) were ligated to adaptor oligonucleotides having 3′ thymine (T) overhangs (see FIG. 21). Prior to the ligation reaction, AMPURE XP beads at 2-fold volume of PCR reaction volume were used to remove single-stranded primers and amplicons generated by asymmetric PCR. Cleaned amplicons were quantified by Agilent Bioanalyzer and mixed with Illumina TRUSEQ library adaptors at an 8:1 ratio. 2 μL of T4 DNA ligase (Enzymatics) and 17.5 μL of 2× ligase buffer (Enzymatics) were added, and the ligation reaction was carried out at room temperature for 15 minutes.

### Unidirectional Adaptor Ligation

In some cases, a modified protocol to improve ligation efficiency and to ensure unidirectional ligation was used. Single base overhang ligation can be less efficient compared to ligation of longer cohesive ends. Additionally, using single base overhang ligation, PCR amplicons can ligate with Illumina TRUSEQ adaptors in either orientation such that, when the ligated product were sequenced, only about half of the sequence reads covered the target sites for copy number calculation. Modifications of the ligation procedure were thus developed to overcome such limitations. First, tag sequences that were 5 nucleotides long were designed to replace the original tag sequence (10 nucleotides long) in the PCR primers (for the competitive PCR above; provided in Table 7 below). The tags were of different sequences for reverse or forward PCR primers and each had a deoxyuridine at the junction between tag sequence and target-specific sequence. The modified primers were used at equal molar ratio in the competitive PCR reaction above.

After PCR amplification, the tags were cleaved from the amplicons by uracil N-glycosylase (UNG; UDG) and EndoVIII digestion, creating a 5 base overhang that selectively ligated the PCR amplicon to universal or indexed adaptors (provided in Table 7 below) with high efficiency (see FIG. 22). Specifically, 1 μL UDG (5 U/μL, NEB) and 5 μL EndoVIII (10 U/μL, NEB) were added to each reaction and incubated at 37° C. for 30 minutes. The reaction was stopped by heating at 95° C. for 10 minutes to inactivate UDG, after which it was gradually cooled to 25° C. The amplicons were cleaned by AMPURE XP beads prior to the ligation reaction.

TABLE 7
Primer and adaptor sequences

| | Forward_Primer | Reverse_Primer |
|---|---|---|
| Target | (SEQ ID NOS 350-362) | (SEQ ID NOS 363-375) |
| ALB | TAGCUGCGTAGCAACCTGTTACATATT | GATCUATACTGAGCAAAGGCAATCAAC |
| APOE | TAGCUCAGTTTCTCCTTCCCCAGAC | GATCUGAATGTGACCAGCAACGCAG |
| RNAseP | TAGCUGGTCAGCTCTTCCCTTCATC | GATCUCCTCCCACATGTAATGTGTTG |
| CDC42EP1 | TAGCUAGCTGGTGCGGAGGGTGGG | GATCUATGGGGGAGATGGCCGGTGGA |
| LDHA | TAGCUGGCCTTTGCAACAAGGATCAC | GATCUCGCAATACTAGAAACCAGGGC |
| MGC15523 | TAGCUTCTGGTGACCCCCGCGCTTC | GATCUCATCTCTGGGTGCGCCTTG |
| POP5 | TAGCUCCCTCCACATCCCGCCATC | GATCUCAGCCGCCTGCTCCATCG |
| SOX14 | TAGCUACGGAATCCCGGCTCTGTG | GATCUCCTTCCTAGTGTGAGAACCG |
| SPN | TAGCUGGCCCTGCTGGCGGTCATA | GATCUTGCTCAGCACGAGGGCCCCA |
| SRY1 | TAGCUAGCAACGGGACCGCTACAG | GATCUTCTAGGTAGGTCTTTGTAGCC |
| SRY2 | TAGCUTAAGTTTCGAACTCTGGCACC | GATCUGAAGCATATGATTGCATTGTCAA |
| TBX3 | TAGCUCTCCTCTTTGTCTCTGCGTG | GATCUTTAATCACCCAGCGCATGGC |
| UTY | TAGCUTGATGCCCGATGCCGCCCTT | GATCUGTCTGTGCTGGGTGTTTTTGC |

Adaptors (SEQ ID NOS 376-378)

| | |
|---|---|
| Universal_adaptor | AATGATACGGCGACCACCGAGATCTACACTCTTTCCCTACACGACGCTCTTCCGATCT |
| Index_linker | GCTCTTCCGATCTATAGCT |
| Index_adaptor | 5′phos/ |
| | GATCGGAAGAGCACACGTCTGAACTCCAGTCACAGTCAACAATCTCGTATGCCGTCTTCTGCTTG |

Pre-annealed index adaptor and index-linker was prepared by mixing at equal molar ratio, heating to 95° C. for 5 minutes, and gradually cooled to 25° C. Universal adaptor and pre-annealed index adaptor at equal molar ratio were mixed with the UDG/EndoVIII-digested PCR amplicons (having 5 nucleotide overhangs). The ratio of adaptor to amplicon varied from 8:1 to 2:1. 2 μL of T4 DNA ligase (Enzymatics) and 17.5 μL of 2× ligase buffer (Enzymatics) were added, and the ligation reaction was carried out at room temperature for 15 minutes.

For both ligation approaches, the ligated product (5 μL) was amplified using Illumina TRUSEQ PCR mixture and primers as specified in Table 8 below. Amplified libraries were purified using AMPURE XP beads to remove free primers/adaptors and DNA fragments of smaller size.

TABLE 8
PCR amplification of ligation products

| | Reagent Volume |
|---|---|
| Reagent | (μL) for n = 1 |
| Water, HPLC grade | 11 |
| TRUSEQ PCR master mix | 20 |
| TRUSEQ PCR primers | 4 |
| Ligation product | 5 |
| Total | 40 |
| PCR Cycling conditions | |
| 98° C., 5 min | |
| 98° C., 10 sec | 10 cycles |
| 65° C., 30 sec | |
| 72° C., 30 sec | |
| 72° C., 3 min | |
| 4° C. hold | |

Amplified libraries were retained on an Illumina flow cell and bridge amplified to generate clusters for subsequent sequencing on Illumina's HISEQ 2000. Use of indexed adaptors allowed for sequencing of multiple samples in a single lane on the flow cell.

### Nucleotide Sequence Read Analysis and Fetal DNA Quantification

Nucleotide sequence reads were analyzed and used to calculate copy number of individual markers and fetal percentage. 50 base pair (bp) nucleotide sequence reads were uniquely aligned to expected chromosome positions, allowing up to 5 mismatches outside the target sites/synthetic target sites. Reads having quality score greater than 13 at the target site with expected target DNA or competitor alleles were used to calculate the copy number of each marker. Specifically, the following formula was used:

$$Copy ( DNA ) = Copy ( \text{?} ) \times \frac{Read \text{?} ( expected DNA allele )}{Read \text{?} ( expected \text{?} allele )}$$

$$\text{?} \text{indicates text missing or illegible when filed}$$

Fetal DNA, Y-chromosome DNA and total DNA copy numbers were represented by the mean value of methylation markers, Y-markers and total DNA markers, respectively. Fetal percentage was calculated according to the following formulas:

$$Fetal Fraction ( \text{?} ) = \frac{mean copy number ( methylation markers )}{mean copy number ( total markers )}$$

$$and$$

$$Fetal Fraction ( Y ) = 2 \times \frac{mean copy number ( Y markers )}{mean copy number ( total markers )}$$

$$Digestion efficiency was calculated by$$

$$digestion efficiency = 1 - \frac{mean copy number ( digestion markers )}{mean copy number ( total markers )}$$

$$\text{?} \text{indicates text missing or illegible when filed}$$

### Results

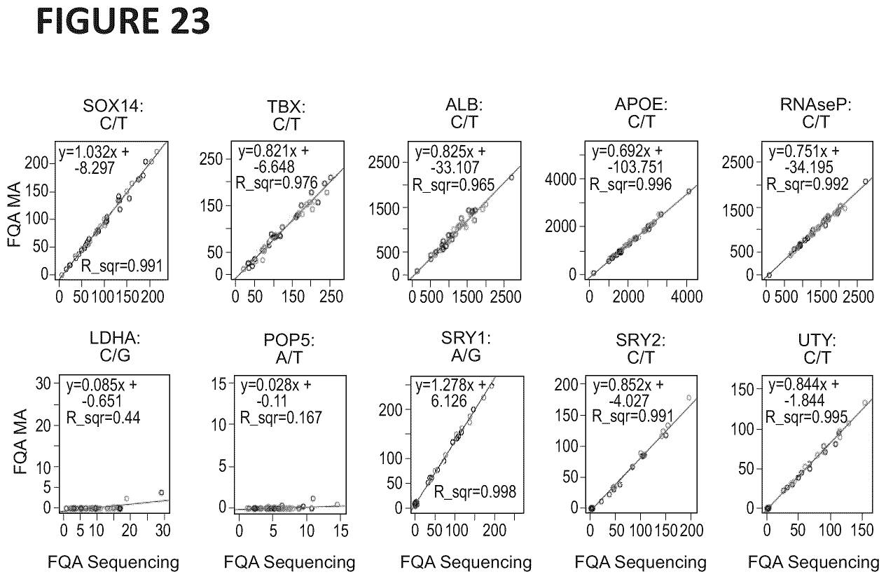

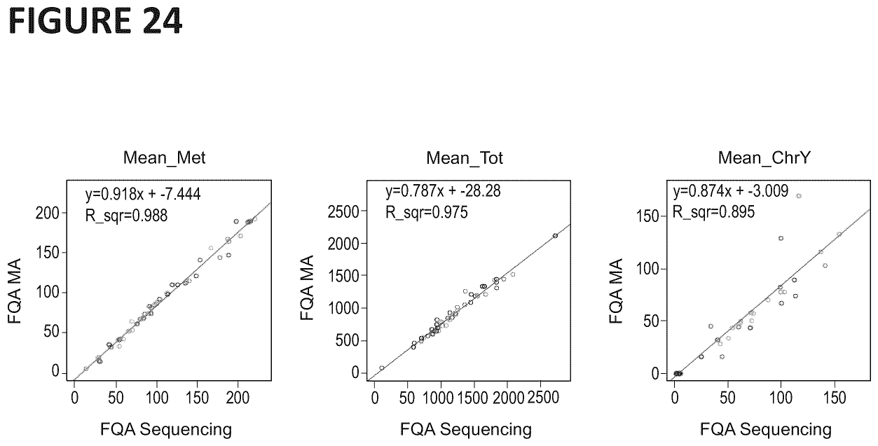

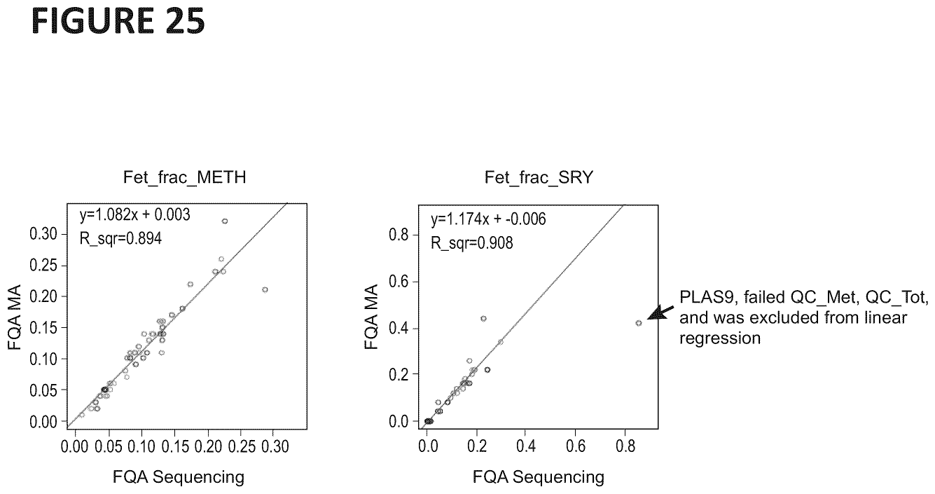

The fetal DNA quantification method using MPSS described in this Example was applied to ccfDNA extracted from 48 plasma samples from pregnant women. The results were compared to those obtained from another method that used mass spectrometry (e.g., MASSARRAY) as a detection method instead of MPSS. The results from both methods were highly correlated (see FIGS. 23 and 24). With exception of digestion markers (LDHA and POP5, which were detected at higher levels by the MPSS method), the R² values were in the range of 0.965-0.998. The fetal fractions derived from methylation markers also were highly correlated between MPSS and mass spectrometry methods (see FIG. 25).

### Example 5: SNP Allele Frequency Based Method for Fetal Fraction Quantification

In this example, single nucleotide polymorphism (SNP) markers were utilized to detect and quantify circulating cell-free (CCF) fetal DNA in maternal plasma (i.e. fetal fraction). In some cases, fetal fraction was determined by measuring single nucleotide polymorphism alleles using a single tube multiplex PCR for amplicon sequencing via massively parallel shotgun sequencing (MPSS).

Advantages of this methodology include, for example: 1) the ability to detect CCF fraction of DNA from both male and female fetuses without prior knowledge of maternal or paternal SNP genotypes; 2) a simplified workflow that generates MPSS ready products without the need for traditional library generation and 3) an ability to perform MPSS fetal fraction quantification on samples multiplexed with genomic libraries on the same flow cell lane.

### Materials and Methods

CCF DNA was extracted from 4 mL plasma from 46 pregnant women using QIAAMP Circulating Nucleic Acid kit in an elution volume of 55 μl. DNA also was extracted from maternal buffy coat samples for confirmation of maternal genotypes. Gestational age at collection ranged from 10-17 weeks. Maternal age ranged from 18-42 years. Ethnic background of samples included African American, Asian, Caucasian and Hispanic ethnicities. 15 μl of CCF DNA underwent PCR for each SNP panel using a single tube multiplex of forward and reverse PCR primers that included adapter sequences to allow secondary amplification with universal PCR primers designed to incorporate index tags. Amplicon libraries with index tags were clustered on the cBOT and sequenced on the HiSeq 2000 for 36 cycles or 27 cycles to generate amplicon sequence reads and 7 cycles to determine the index tag sequence. Reads were aligned to the human genome (hg19) and matched read counts for expected SNP alleles were used to calculate the allele ratio of each SNP within each CCF DNA. 15 μl of CCF DNA also was used for quantification of fetal fraction by fetal specific methylation patterns for comparison with SNP based quantification.

### Detection of Paternally Inherited Alleles

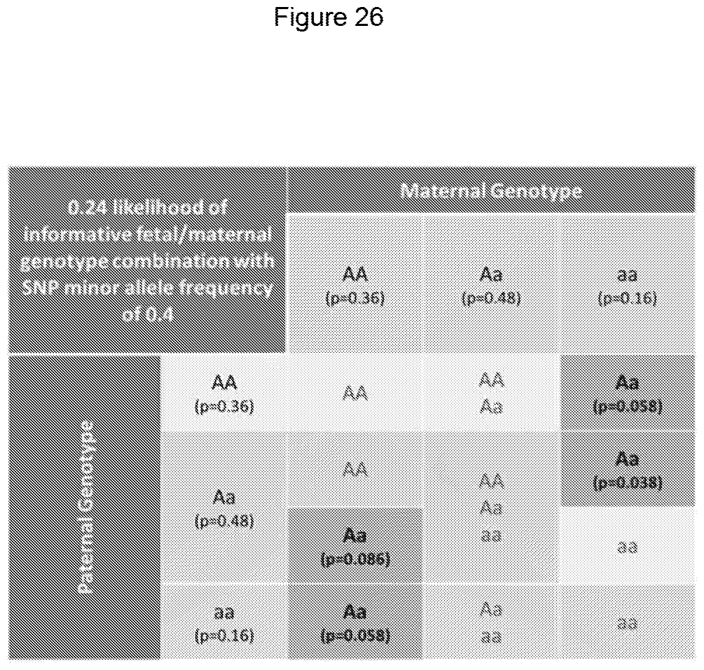

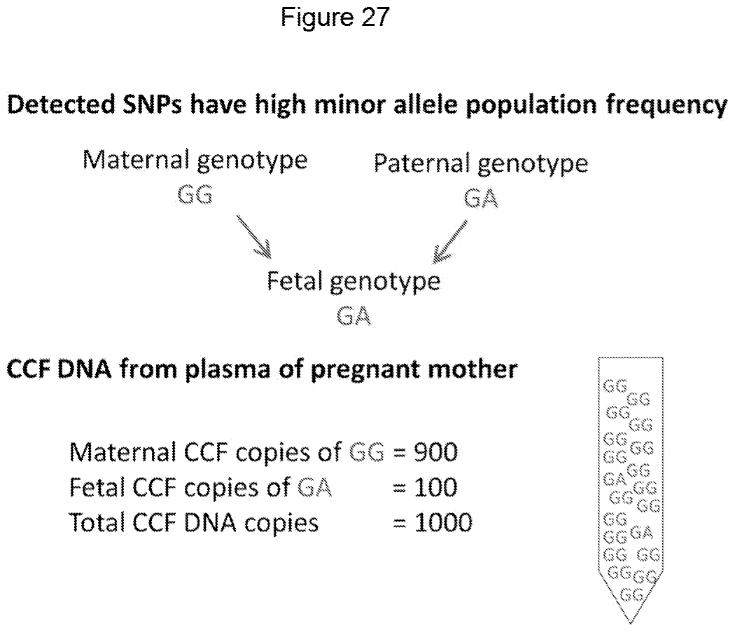

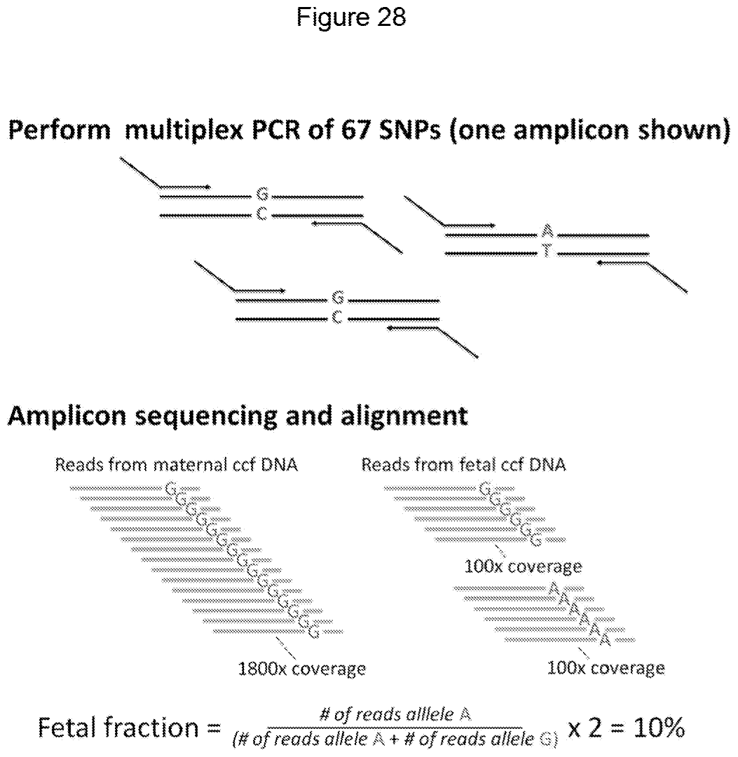

CCF fetal DNA in maternal plasma contains both maternally and paternally inherited DNA (e.g., SNP alleles). Detection of paternal SNP alleles not present in the maternal genome can allow confirmation of the presence of fetal DNA. Additionally, quantification of paternal:maternal SNP allele ratios can provide for a determination of fetal DNA fraction in maternal plasma. The likelihood of detecting a paternally inherited allele at a single locus is dependent upon allele frequency and individual inheritance patterns. FIG. 26, for example, provides a summary of expected genotypes and the associated population frequency of each genotype based a SNP having a minor allele population frequency of 0.4. A SNP with a high minor allele frequency may increase the chance that paternal and maternal alleles will differ at a given SNP locus. Provided enough SNPs are interrogated, a high probability can be established that the fetus will contain some paternal alleles that differ from the maternal alleles. Thus, use of multiple SNP alleles increases the likelihood of informative fetal and maternal genotype combinations. Often, no prior knowledge of the paternal genotypes is required because paternal alleles can be inferred by the presence of non-maternal alleles in the maternal/fetal cell free DNA mixture. FIGS. 27 and 28 show how fetal fraction can be calculated using SNP allele frequency.

### SNP Panels

High minor allele frequency SNPs that contain only 2 known alleles were identified. Two panels of SNPs were generated: a 67 SNP panel (SNP panel 1) and an 86 SNP panel (SNP panel 2). Individual SNP identifiers for each panel are provided in Table 9A and Table 10A below. Tables 9B and 10B include chromosome identity for each SNP.

TABLE 9A
SNP Panel 1

| | | | | |
|---|---|---|---|---|
| rs10413687 | rs2001778 | rs4453265 | rs539344 | rs7176924 |
| rs10949838 | rs2323659 | rs447247 | rs551372 | rs7525374 |
| rs1115649 | rs2427099 | rs4745577 | rs567681 | rs870429 |
| rs11207002 | rs243992 | rs484312 | rs585487 | rs949312 |
| rs11632601 | rs251344 | rs499946 | rs600933 | rs9563831 |
| rs11971741 | rs254264 | rs500090 | rs619208 | rs970022 |
| rs12660563 | rs2827530 | rs500399 | rs622994 | rs985462 |
| rs13155942 | rs290387 | rs505349 | rs639298 | |
| rs1444647 | rs321949 | rs505662 | rs642449 | |
| rs1572801 | rs348971 | rs516084 | rs6700732 | |
| rs17773922 | rs390316 | rs517316 | rs677866 | |
| rs1797700 | rs3944117 | rs517914 | rs683922 | |
| rs1921681 | rs425002 | rs522810 | rs686851 | |
| rs1958312 | rs432586 | rs531423 | rs6941942 | |
| rs196008 | rs444016 | rs537330 | rs7045684 | |

TABLE 9B
SNP Panel 1

| SNP_ID | Chromosome |
|---|---|
| rs10413687 | chr19 |
| rs10949838 | chr7 |
| rs1115649 | chr21 |
| rs11207002 | chr1 |
| rs11632601 | chr15 |
| rs11971741 | chr7 |
| rs12660563 | chr6 |
| rs13155942 | chr5 |
| rs1444647 | chr12 |
| rs1572801 | chr6 |
| rs17773922 | chr19 |
| rs1797700 | chr12 |
| rs1921681 | chr4 |
| rs1958312 | chr14 |
| rs196008 | chr16 |
| rs2001778 | chr11 |
| rs2323659 | chr17 |
| rs2427099 | chr20 |
| rs243992 | chr4 |
| rs251344 | chr5 |
| rs254264 | chr19 |
| rs2827530 | chr21 |
| rs290387 | chr20 |
| rs321949 | chr19 |
| rs348971 | chr2 |
| rs390316 | chr14 |
| rs3944117 | chr7 |
| rs425002 | chr4 |
| rs432586 | chr12 |
| rs444016 | chr5 |
| rs4453265 | chr11 |
| rs447247 | chr6 |
| rs4745577 | chr9 |
| rs484312 | chr13 |
| rs499946 | chr7 |
| rs500090 | chr11 |
| rs500399 | chr10 |
| rs505349 | chr11 |
| rs505662 | chr6 |
| rs516084 | chr1 |
| rs517316 | chr1 |
| rs517914 | chr4 |
| rs522810 | chr13 |
| rs531423 | chr1 |
| rs537330 | chr8 |
| rs539344 | chr19 |
| rs551372 | chr11 |
| rs567681 | chr11 |
| rs585487 | chr19 |
| rs600933 | chr1 |
| rs619208 | chr11 |
| rs622994 | chr13 |
| rs639298 | chr1 |
| rs642449 | chr1 |
| rs6700732 | chr1 |
| rs677866 | chr13 |
| rs683922 | chr15 |
| rs686851 | chr6 |
| rs6941942 | chr6 |
| rs7045684 | chr9 |
| rs7176924 | chr15 |
| rs7525374 | chr1 |
| rs870429 | chr3 |
| rs949312 | chr18 |
| rs9563831 | chr13 |
| rs970022 | chr4 |
| rs985462 | chr10 |

TABLE 10A
SNP Panel 2

| | | | | |
|---|---|---|---|---|
| rs1005241 | rs1432515 | rs2906237 | rs654065 | rs849084 |
| rs1006101 | rs1452396 | rs2929724 | rs6576533 | rs873870 |
| rs10745725 | rs1518040 | rs3742257 | rs6661105 | rs9386151 |
| rs10776856 | rs16853186 | rs3764584 | rs669161 | rs9504197 |
| rs10790342 | rs1712497 | rs3814332 | rs6703320 | rs9690525 |
| rs11076499 | rs1792205 | rs4131376 | rs675828 | rs9909561 |
| rs11103233 | rs1863452 | rs4363444 | rs6814242 | |
| rs11133637 | rs1991899 | rs4461567 | rs6989344 | |
| rs11974817 | rs2022958 | rs4467511 | rs7120590 | |
| rs12102203 | rs2099875 | rs4559013 | rs7131676 | |
| rs12261 | rs2108825 | rs4714802 | rs7214164 | |
| rs12460763 | rs2132237 | rs4775899 | rs747583 | |
| rs12543040 | rs2195979 | rs4817609 | rs768255 | |
| rs12695642 | rs2248173 | rs488446 | rs768708 | |
| rs13137088 | rs2250246 | rs4950877 | rs7828904 | |
| rs13139573 | rs2268697 | rs530913 | rs7899772 | |
| rs1327501 | rs2270893 | rs6020434 | rs7900911 | |
| rs13438255 | rs244887 | rs6442703 | rs7925270 | |
| rs1360258 | rs2736966 | rs6487229 | rs7975781 | |
| rs1421062 | rs2851428 | rs6537064 | rs8111589 | |

TABLE 10B
SNP Panel 2

| SNP_ID | Chromosome |
|---|---|
| rs1518040 | chr1 |
| rs16853186 | chr1 |
| rs2268697 | chr1 |
| rs3814332 | chr1 |
| rs4363444 | chr1 |
| rs4950877 | chr1 |
| rs6661105 | chr1 |
| rs6703320 | chr1 |
| rs1432515 | chr2 |
| rs12695642 | chr3 |
| rs2132237 | chr3 |
| rs6442703 | chr3 |
| rs13137088 | chr4 |
| rs13139573 | chr4 |
| rs1452396 | chr4 |
| rs1712497 | chr4 |
| rs4461567 | chr4 |
| rs4467511 | chr4 |
| rs6537064 | chr4 |
| rs6814242 | chr4 |
| rs747583 | chr4 |
| rs1006101 | chr5 |
| rs11133637 | chr5 |
| rs2929724 | chr5 |
| rs4559013 | chr5 |
| rs4714802 | chr6 |
| rs669161 | chr6 |
| rs9386151 | chr6 |
| rs9504197 | chr6 |
| rs11974817 | chr7 |
| rs13438255 | chr7 |
| rs2736966 | chr7 |
| rs2906237 | chr7 |
| rs4131376 | chr7 |
| rs849084 | chr7 |
| rs9690525 | chr7 |
| rs12543040 | chr8 |
| rs1863452 | chr8 |
| rs2022958 | chr8 |
| rs6989344 | chr8 |
| rs7828904 | chr8 |
| rs10776856 | chr9 |
| rs11103233 | chr9 |
| rs1327501 | chr9 |
| rs1360258 | chr9 |
| rs1421062 | chr10 |
| rs2248173 | chr10 |
| rs768255 | chr10 |
| rs7899772 | chr10 |
| rs7900911 | chr10 |
| rs10790342 | chr11 |
| rs1792205 | chr11 |
| rs1991899 | chr11 |
| rs2099875 | chr11 |
| rs2851428 | chr11 |
| rs488446 | chr11 |
| rs7120590 | chr11 |
| rs7131676 | chr11 |
| rs768708 | chr11 |
| rs7925270 | chr11 |
| rs10745725 | chr12 |
| rs2250246 | chr12 |
| rs2270893 | chr12 |
| rs6487229 | chr12 |
| rs7975781 | chr12 |
| rs12261 | chr13 |
| rs3742257 | chr13 |
| rs675828 | chr13 |
| rs12102203 | chr15 |
| rs4775899 | chr15 |
| rs6576533 | chr15 |
| rs11076499 | chr16 |
| rs244887 | chr16 |
| rs654065 | chr16 |
| rs7214164 | chr17 |
| rs9909561 | chr17 |
| rs12460763 | chr19 |
| rs2108825 | chr19 |
| rs2195979 | chr19 |
| rs3764584 | chr19 |
| rs8111589 | chr19 |
| rs873870 | chr19 |
| rs530913 | chr20 |
| rs6020434 | chr20 |
| rs4817609 | chr21 |
| rs1005241 | chr22 |

### Generation of Illumina Sequencer Ready Amplicons

For SNP panel 1, PCR primers were designed to amplify the 67 targeted SNPs plus a flanking region of 35 base pairs (bp) surrounding the SNP site. The 67 targeted regions were amplified in a single multiplex reaction. For SNP panel 2, PCR primers were designed to amplify the 86 targeted SNPs plus a flanking region of 26 base pairs (bp) surrounding the SNP site. The 86 targeted regions were amplified in a single multiplex reaction.

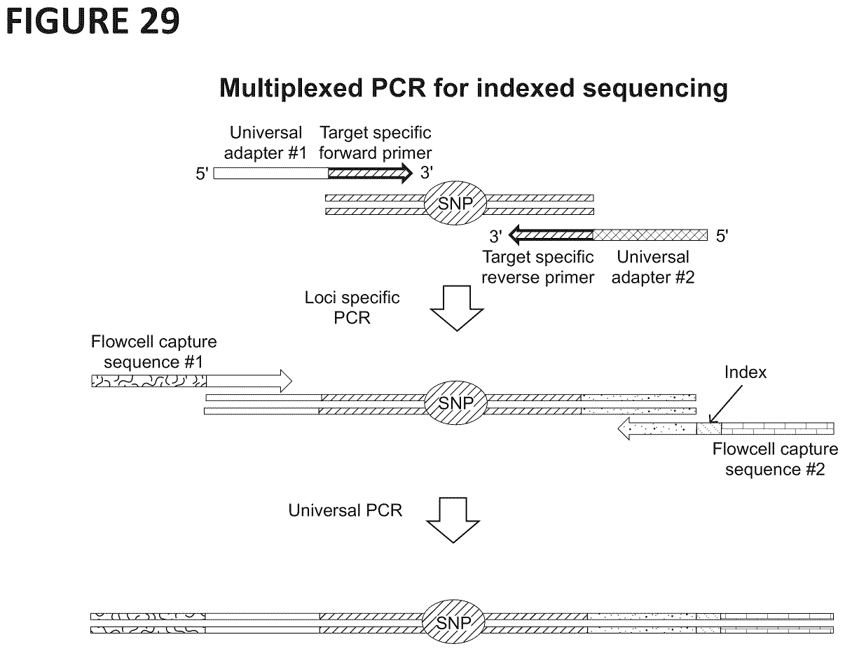

PCR primers were modified such that Illumina sequencing adapters could be added via universal tag sequences incorporated onto the 5′ end of the SNP-specific PCR primers. Illumina tags were added using two separate PCR reactions (see FIG. 29 and Table 11 below): 1) a loci-specific PCR which incorporated a section of the Illumina sequencing adapters followed by 2) a universal PCR whose primers annealed to the tags in the loci-specific PCR to complete the addition of the adapters whilst allowing the addition of a sample specific index sequence via the reverse primer in the universal PCR. A 3^{rd} single cycle PCR was performed to remove heteroduplex secondary structure that can arise in the amplicons during the universal PCR stage due to cross-annealing of shared adapter sequences between different amplicons in the same multiplex. Loci-specific PCR and universal PCR were performed under standard conditions using primers synthesized from Integrated DNA Technologies (IDT; Coralville, IA) with no special modifications.

TABLE 11
Sequencing adaptors, loci specific PCR primer tags
and universal PCR primer tags (SEQ ID NOS 379-386)

| Name | Sequence |
|---|---|
| TRUSEQ P5 Adapter | 5′-AATGATACGGCGACCA |
| | CCGAGATCTACACTCTTTC |
| | CCTACACGACGCTCTTCCG |
| | ATCT-3′ |
| TRUSEQ Read 1 | 5′-ACACTCTTTCCCTACA |
| sequencing primer | CGACGCTCTTCCGATCT-3′ |
| TRUSEQ P7 adapter, | 5′-GATCGGAAGAGCACAC |
| Index 13 | GTCTGAACTCCAGTCACAG |
| | TCAAATCTCGTATGCCGTC |
| | TTCTGCTTG-3′ |
| TRUSEQ index read | 5′-GATCGGAAGAGCACAC |
| primer | GTCTGAACTCCAGTCAC-3′ |
| Loci PCR forward tag | 5′-TCTTTCCCTACACGAC |
| | GCTCTTCCGATCT-3′ |
| Loci PCR reverse tag | 5′-GTGACTGGAGTTCAGA |
| | CGTGTGCTCTTCCGATCT-3′ |
| UNIV PCR forward | 5′-AATGATACGGCGACCA |
| primer | CCGAGATCTACACTCTTTC |
| | CCTACACGACGCTC-3′ |
| UNIV PCR reverse index | 5′-CAAGCAGAAGACGGCA |
| 13 primer | TACGAGATTTGACTGTGAC |
| | TGGAGTTCAGACGTG-3′ |

Amplicon Sequencing by/Illumina NGS

Universal PCR products were quantified using standard DNA fragment analysis methods such as Caliper LabChip GX or Agilent Bioanalyzer. The sequencer-ready amplicons from up to 12 samples were pooled and sequenced on an Illumina HISEQ apparatus. For SNP panel 1, 36 cycles were used to sequence the target SNP plus the 35 bp flanking region. For SNP panel 2, 27 cycles were used to sequence the target SNP plus the 26 bp flanking region. Samples were de-mulitplexed using a 6 bp index identifier incorporated at the universal PCR stage.

### Assignment of Informative Alleles and Fetal Fraction Determination

Reads were aligned to the human genome (hg19) with up to 3 mismatches in each read to allow for sequencing error and variant alleles at target SNP position. The frequency of each SNP allele was determined by counting the number of reads having the allele of interest and dividing it by the total number of reads for each SNP locus (i.e., (#reads allele 1)/(#reads allele 1+#reads allele 2)). Based on the frequency value generated from this data, the sequenced genotypes were assigned as Type 0 non-informative genotypes, Type 1 informative genotypes or Type 2 informative genotypes. A Type 0 non-informative genotype is a fetal genotype that cannot be distinguished from the maternal genotype because the fetus has the same genotype as the mother (e.g., mother is “Aa” and fetus is “Aa”). A Type I informative genotype is the situation where the mother is homozygous (AA) and the fetus is heterozygous (Aa). This genotype is informative because allele “a” is from the father. The frequency of a Type 1 informative allele can be indicative of the percentage fetal DNA in the mixture. A Type 2 informative genotype is the situation where the mother is heterozygous (Aa) and the fetus is homozygous (AA). The genotype is informative because the frequency of the maternal allele “a” will deviate from the expected Mendelian frequency of 0.5 when there is fetal DNA contributing additional “A” alleles. This deviation in value from 0.5 can be used to compute the fetal fraction.

Allele frequencies for each of the SNPs was calculated for each sample based on the number of reads containing each allele, as described above. Variation of expected allele frequency could be due to the presence of fetal DNA with a different paternal allele or could be due to mis-incorporated sequences by the Illumina Sequencer (e.g., background noise). In some cases, the amount of background noise associated with each particular SNP amplicon was determined to establish a dynamic cutoff value for each SNP. Maternal DNA (i.e. buffy coat) samples were sequenced and the deviations from the expected Mendelian ratios of 1 for homozygotes and 0.5 for heterozygotes were observed. From these values a median-adjusted deviation (MAD score) was identified for each SNP assay. In some cases, a genotype was identified as being a Type I informative genotype when the paternal allele frequency measured was greater than 3×MAD score. In some cases, multiple Type 1 informative genotypes were identified and an average allele frequency was determined. Fetal fraction was calculated by multiplying the average Type 1 informative allele frequency by 2. For example, an average informative allele frequency of 4.15% indicated a fetal fraction of 8.3%. Fetal Fraction also can be calculated from Type 2 informative genotypes by determining maternal allele “a” frequencies deviating from 0.5 by greater than 3×MAD, for example. Fetal fraction can be identified by multiplying this deviation by 2.

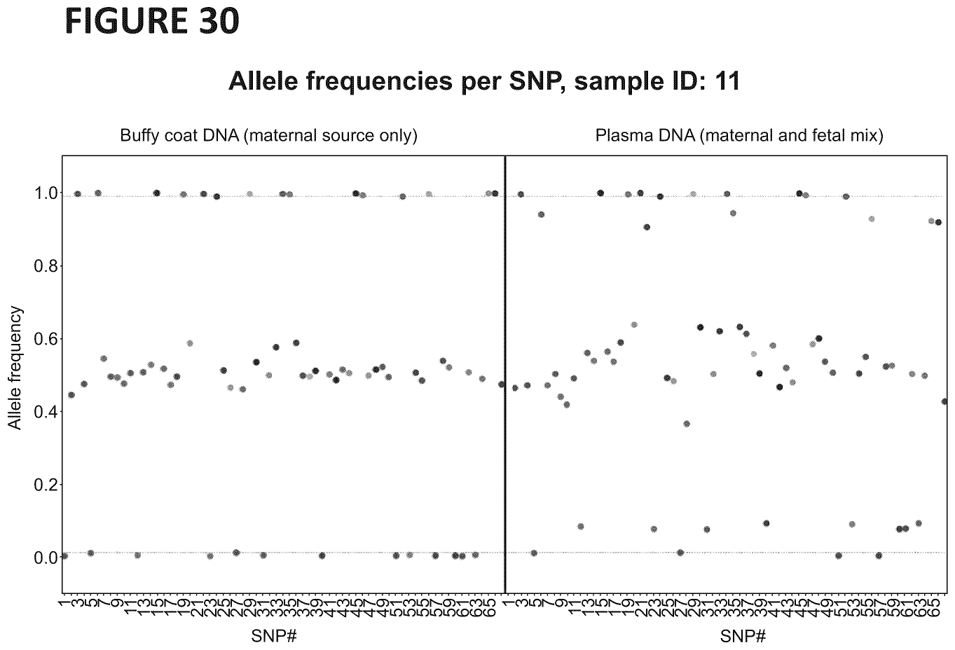

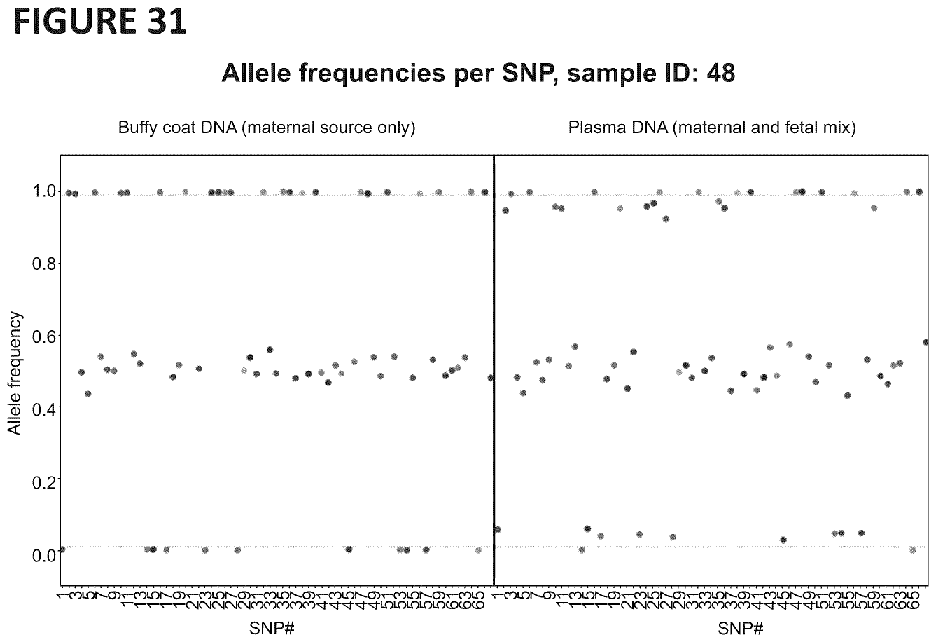

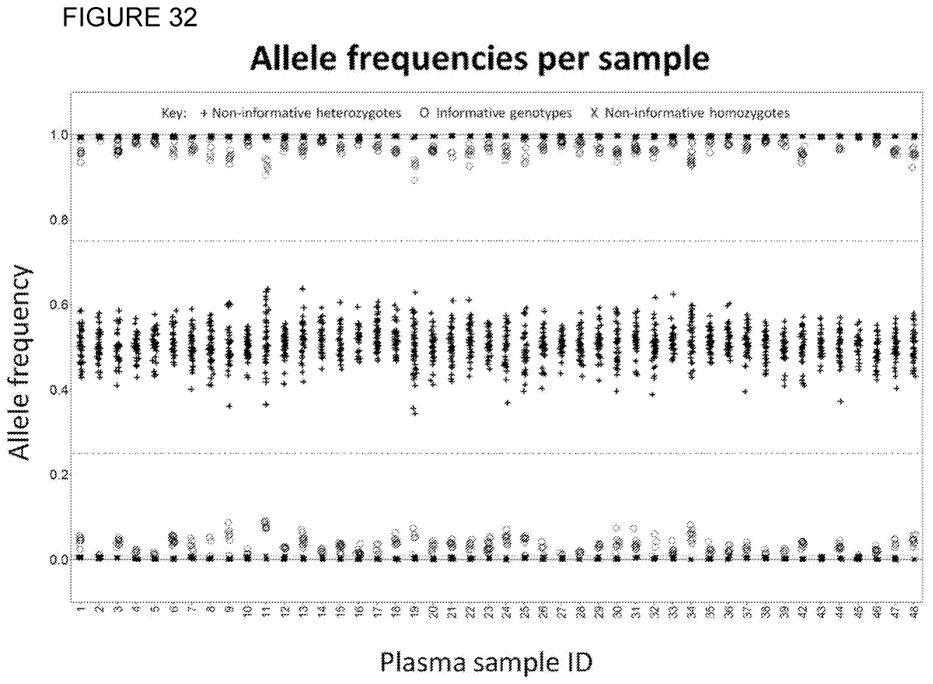

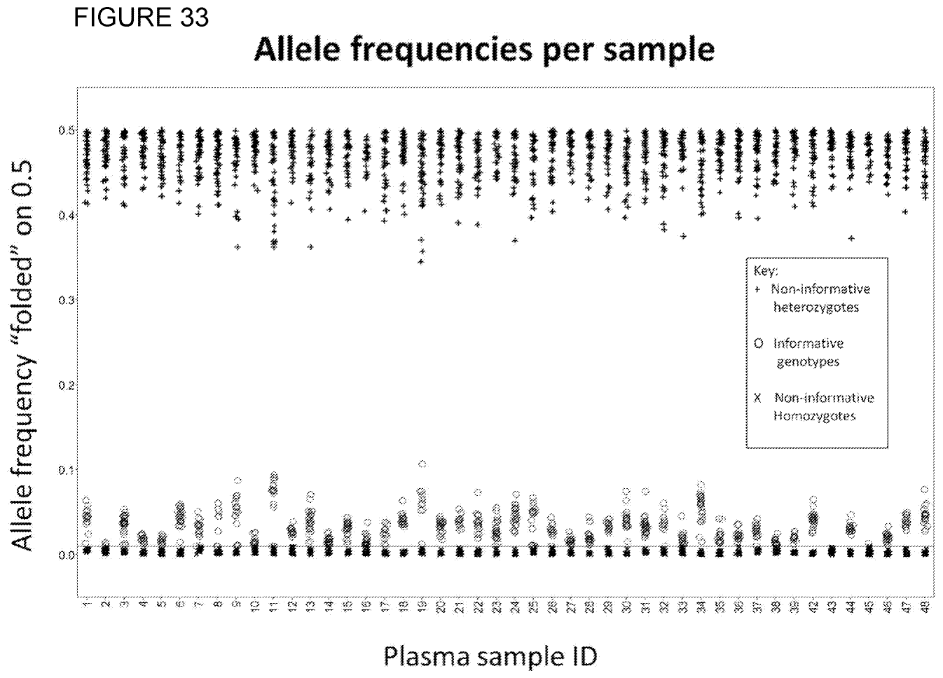

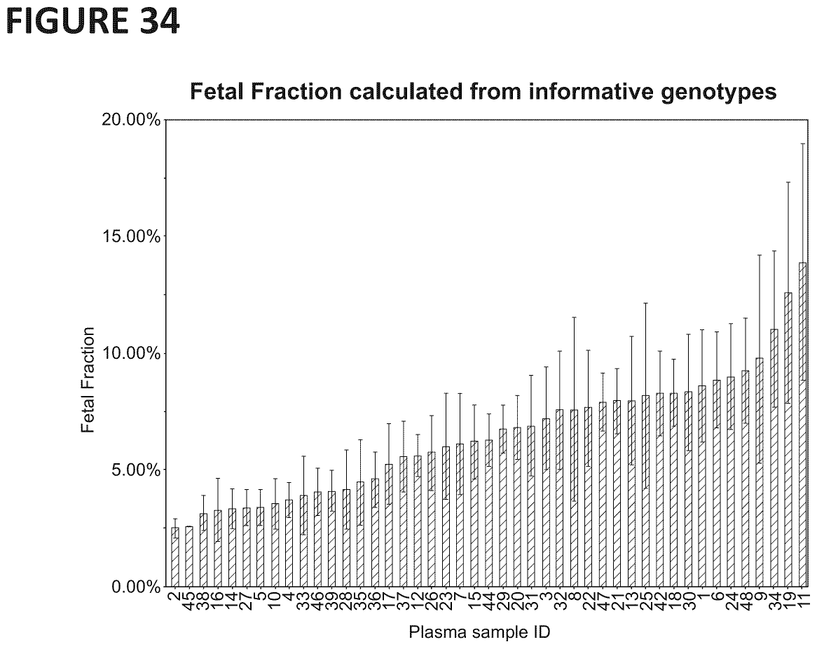

In some cases, informative genotypes were assigned without prior knowledge of maternal or paternal genotypes. Allele frequencies for each SNP (of SNP panel 1) were plotted as shown in FIG. 30 and FIG. 31 for two of the 46 samples tested. Homozygous allele frequencies in maternal buffy coat were close to 0 or 1. Type 1 informative SNPs were identified by allele frequencies that deviated from the expected allele frequency of 0 or 1 due to the presence of a paternal allele from the fetus. The size of the deviation was dependent on the size of the fetal fraction of CCF DNA. A maximum background allele frequency of 0.007 was observed for maternal buffy coat DNA. For this approach, fixed cutoff frequency value of 0.01 was used to distinguish non-informative homozygotes from informative genotypes in plasma samples (see FIGS. 32 and 33, showing the assignment of certain Type 1 informative genotypes). A fixed cutoff value of 0.25 was used to distinguish non-informative heterozygotes from other genotypes. Fetal fractions were calculated for 46 plasma samples by taking the mean of the informative genotype allele frequencies and multiplying this value by 2. Informative genotypes assigned per sample ranged from 1 to 26. Fetal fractions ranged from 2.5% to 14% (see FIG. 34).

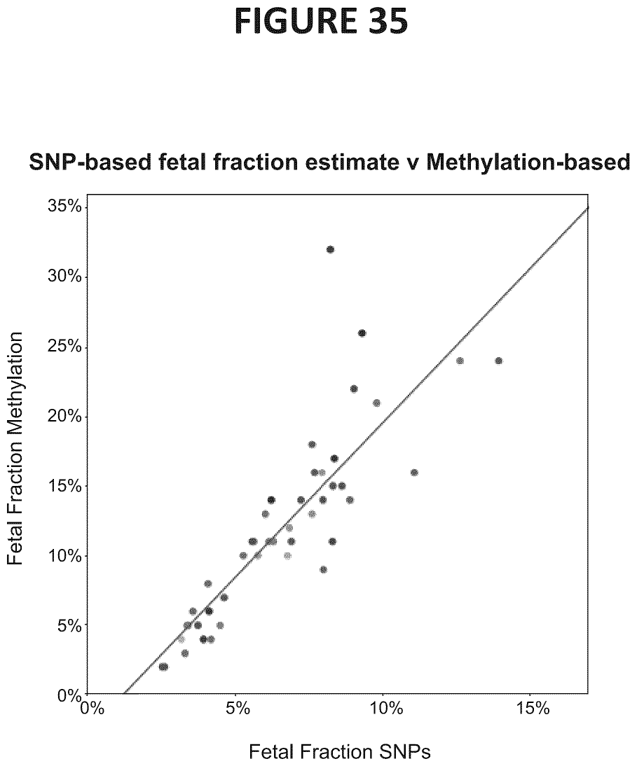

To assess performance of the above method, fetal fractions also were determined for the 46 plasma samples using a differential methylation-based fetal quantifier assay. SNP-based fetal fraction estimates showed a linear association with the methylation-based estimates (r²=0.72). FIG. 35 shows linear regression of fetal fraction estimate methods as a diagonal line.

### Amplicon Sequence Coverage

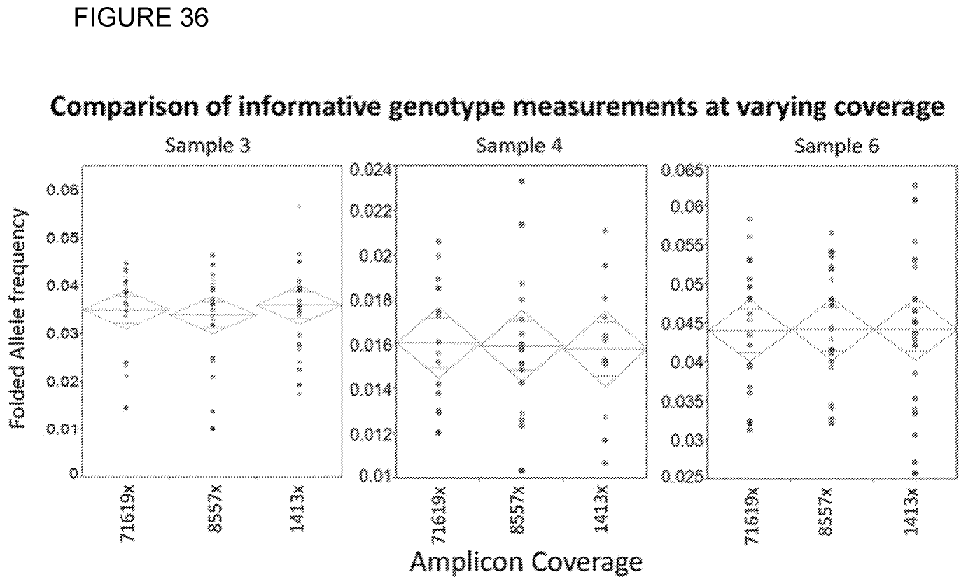

Various amounts of SNP amplicon libraries were combined (i.e. diluted) with TRUSEQ libraries to demonstrate that allele frequency determinations can be made at varying levels of amplicon sequence coverage. SNP amplicon libraries from 6 plasma samples and 6 buffy coat samples were combined with 11 TRUSEQ libraries and co-sequenced on a HISEQ 2000 apparatus in the same flowcell lane. Percent (%) of SNP amplicon library combined with TRUSEQ libraries ranged from 50% to 0.8%. After alignment coverage per SNP for each amplicon library ranged from 71619x per SNP (50% amplicon library) to 1413x per SNP (0.8% amplicon library). Fetal fraction estimates were not significantly different even at lowest coverage level (see FIG. 36). These findings indicate that less than 1% of the flowcell clusters on a HISEQ 2000 apparatus can be used to co-sequence amplicon libraries and that high levels of sample multiplexing (e.g., greater than 96) can be achieved.

### Example 6: Examples of Embodiments

Provided hereafter are non-limiting examples of certain embodiments of the technology.

A1. A method for determining the amount of fetal nucleic acid in a sample comprising:
(a) contacting a sample nucleic acid with one or more agents that differentially modify methylated nucleic acid and unmethylated nucleic acid, which sample nucleic acid comprises differentially methylated fetal nucleic acid and maternal nucleic acid, the combination of the fetal nucleic acid and the maternal nucleic acid comprising total nucleic acid in the sample, thereby generating differentially modified sample nucleic acid;
(b) contacting under amplification conditions the differentially modified sample nucleic acid with:
   (i) a first set of amplification primers that specifically amplify a first region in sample nucleic acid comprising one or more loci that are differentially methylated between the fetal nucleic acid and maternal nucleic acid, and
   (ii) a second set of amplification primers that amplify a second region in the sample nucleic acid allowing for a determination of total nucleic acid in the sample, wherein the first region and the second region are different, thereby generating fetal nucleic acid amplification products and total nucleic acid amplification products;
(c) incorporating adaptor oligonucleotides into the amplification products in (b); thereby generating adaptor-modified amplification products;
(d) obtaining nucleotide sequences of the adaptor-modified amplification products in (c) by a sequencing process, thereby generating sequence reads;
(e) quantifying the sequence reads; and
(f) determining the amount of fetal nucleic acid in the sample based on a quantification of the sequence reads in (e).

A2. The method of embodiment A1, wherein the first region comprises one or more loci which each contain a restriction site for a methylation-sensitive restriction enzyme.

A3. The method of embodiment A2, wherein the one or more agents that differentially modify methylated nucleic acid and unmethylated nucleic acid comprise one or more methylation sensitive restriction enzymes.

A4. The method of embodiment A2 or A3, wherein the second region comprises one or more loci which do not contain a restriction site for a methylation-sensitive restriction enzyme.

A5. The method of embodiment A1, wherein the one or more agents that differentially modify methylated nucleic acid and unmethylated nucleic acid comprise bisulfite.

A6. The method of any one of embodiments A1 to A5, wherein the adaptor oligonucleotides are incorporated into the amplification products by ligation.

A7. The method of embodiment A6, wherein the ligation is unidirectional ligation.

A8. The method of any one of embodiments A1 to A5, wherein the adaptor oligonucleotides are incorporated into the amplification products using amplification primers comprising the adaptor oligonucleotide sequences.

A9. The method of any one of embodiments A1 to A8, wherein the adaptor oligonucleotides comprise one or more index sequences.

A10. The method of embodiment A9, wherein the one or more index sequences comprise a sample-specific index.

A11. The method of embodiment A9, wherein the one or more index sequences comprise an aliquot-specific index.

A12. The method of any one of embodiments A1 to A11, wherein at least one of the one or more loci in the first region comprises a nucleotide sequence selected from among SEQ ID NOs:1-261, or a fragment thereof.

A13. The method of embodiment A12, wherein at least one of the one or more loci in the first region comprises a nucleotide sequence selected from among SEQ ID NOs:1-89, or a fragment thereof.

A14. The method of embodiment A12, wherein at least one of the one or more loci in the first region comprises a nucleotide sequence selected from among SEQ ID NOs:90-261, or a fragment thereof.

A15. The method of embodiment A12, wherein at least one of the one or more loci in the first region comprises a nucleotide sequence selected from among SEQ ID NOs:1-59 and SEQ ID NOs:86-89, or a fragment thereof.

A16. The method of embodiment A12, wherein at least one of the one or more loci in the first region comprises a nucleotide sequence selected from among SEQ ID NOs:1-59, or a fragment thereof.

A17. The method of embodiment A12, wherein at least one of the one or more loci in the first region comprises a nucleotide sequence selected from among SEQ ID NO:42, SEQ ID NO:52, SEQ ID NO:154, SEQ ID NO:158 and SEQ ID NO:163.

A18. The method of any one of embodiments A1 to A17, wherein the sequencing process is a sequencing by synthesis method.

A19. The method of any one of embodiments A1 to A18, wherein the sequencing process is a reversible terminator-based sequencing method.

A20. The method of any one of embodiments A1 to A19, wherein the amount of fetal nucleic acid determined is the fraction of fetal nucleic acid in the sample based on the amount of each of the fetal nucleic acid amplification products and total nucleic acid amplification products.

A21. The method of embodiment A20, wherein the fraction of fetal nucleic acid is a ratio of fetal nucleic acid amplification product amount to total nucleic acid amplification product amount.

A22. The method of any one of embodiments A1 to A21, further comprising contacting under amplification conditions the nucleic acid sample with a third set of amplification primers that amplify a third region in the sample nucleic acid allowing for a determination of the presence or absence of fetal specific nucleic acid.

A23. The method of embodiment A22, wherein the fetal specific nucleic acid is Y chromosome nucleic acid.

A24. The method of embodiment A23, wherein the third region comprises one or more loci within chromosome Y.

A25. The method of any one of embodiments A3 to A24, further comprising contacting under amplification conditions the nucleic acid sample with a fourth set of amplification primers that amplify a fourth region in the sample nucleic acid allowing for a determination of the amount of digested or undigested nucleic acid, as an indicator of digestion efficiency.

A26. The method of embodiment A25, wherein the fourth region comprises one or more loci present in both fetal nucleic acid and maternal nucleic acid and unmethylated in both fetal nucleic acid and maternal nucleic acid.

A27. The method of any one of embodiments A1 to A26, further comprising contacting under amplification conditions the nucleic acid sample with a predetermined copy number of one or more first competitor oligonucleotides that compete with the first region for hybridization of primers of the first amplification primer set.

A28. The method of any one of embodiments A1 to A27, further comprising contacting under amplification conditions the nucleic acid sample with a predetermined copy number of one or more second competitor oligonucleotides that compete with the second region for hybridization of primers of the second amplification primer set.

A29. The method of any one of embodiments A22 to A28, further comprising contacting under amplification conditions the nucleic acid sample with a predetermined copy number of one or more third competitor oligonucleotides that compete with the third region for hybridization of primers of the third amplification primer set.

A30. The method of any one of embodiments A25 to A29, further comprising contacting under amplification conditions the nucleic acid sample with a predetermined copy number of one or more fourth competitor oligonucleotides that compete with the fourth region for hybridization of primers of the fourth amplification primer set.

A31. The method of any one of embodiments A27 to A30, wherein the amount of fetal nucleic acid determined is the copy number of fetal nucleic acid based on the amount of competitor oligonucleotide used.

A32. The method of any one of embodiments A1 to A26, wherein the amount of fetal nucleic acid determined is the copy number of fetal nucleic acid based on a quantification of sequence reads.

A33. The method of any one of embodiments A1 to A32, wherein the sample nucleic acid is extracellular nucleic acid.

A34. The method of any one of embodiments A1 to A33, wherein the nucleic acid sample is obtained from a pregnant female subject.

A35. The method of embodiment A34, wherein the subject is human.

A36. The method of any one of embodiments A1 to A35, wherein the sample nucleic acid is from plasma or serum.

A37. The method of any one of embodiments A1 to A36, wherein two or more independent loci in the first region are assayed.

A38. The method of any one of embodiments A1 to A37, wherein the amount of fetal nucleic acid is substantially equal to the amount of fetal nucleic acid determined using a mass spectrometry method.

A39. The method of any one of embodiments A1 to A38, wherein the amount of fetal nucleic acid is determined with an R² value of 0.97 or greater when compared to an amount of fetal nucleic acid determined using a mass spectrometry method.

B1. A method for determining the amount of fetal nucleic acid in a sample comprising:
(a) contacting a sample nucleic acid with one or more methylation sensitive restriction enzymes, which sample nucleic acid comprises differentially methylated fetal nucleic acid and maternal nucleic acid, the combination of the fetal nucleic acid and the maternal nucleic acid comprising total nucleic acid in the sample, thereby generating differentially digested sample nucleic acid;
(b) contacting under amplification conditions the digested sample nucleic acid with:
   (i) a first set of amplification primers that specifically amplify a first region in sample nucleic acid comprising one or more loci that are differentially methylated between the fetal nucleic acid and maternal nucleic acid, and
   (ii) a second set of amplification primers that amplify a second region in the sample nucleic acid allowing for a determination of total nucleic acid in the sample, wherein the first region and the second region are different, thereby generating fetal nucleic acid amplification products and total nucleic acid amplification products;
(c) incorporating adaptor oligonucleotides into the amplification products in (b); thereby generating adaptor-modified amplification products;
(d) obtaining nucleotide sequences of the adaptor-modified amplification products in (c) by a sequencing process, thereby generating sequence reads;
(e) quantifying the sequence reads; and
(f) determining the amount of fetal nucleic acid in the sample based on a quantification of the sequence reads in (e).

B2. The method of embodiment 1, wherein the adaptor oligonucleotides are incorporated into the amplification products by ligation.

B3. The method of embodiment B2, wherein the ligation is unidirectional ligation.

B4. The method of any one of embodiments 1 to B3, wherein the adaptor oligonucleotides are incorporated into the amplification products using amplification primers comprising the adaptor oligonucleotide sequences.

B5. The method of any one of embodiments 1 to B4, wherein the adaptor oligonucleotides comprise one or more index sequences.

B6. The method of embodiment B5, wherein the one or more index sequences comprise a sample-specific index.

B7. The method of embodiment B5, wherein the one or more index sequences comprise an aliquot-specific index.

B8. The method of any one of embodiments 1 to B7, wherein at least one of the one or more loci in the first region comprises a nucleotide sequence selected from among SEQ ID NOs:1-261, or a fragment thereof.

B9. The method of embodiment B8, wherein at least one of the one or more loci in the first region comprises a nucleotide sequence selected from among SEQ ID NOs:1-89, or a fragment thereof.

B10. The method of embodiment B8, wherein at least one of the one or more loci in the first region comprises a nucleotide sequence selected from among SEQ ID NOs:90-261, or a fragment thereof.

B11. The method of embodiment B8, wherein at least one of the one or more loci in the first region comprises a nucleotide sequence selected from among SEQ ID NOs:1-59 and SEQ ID NOs:86-89, or a fragment thereof.

B12. The method of embodiment B8, wherein at least one of the one or more loci in the first region comprises a nucleotide sequence selected from among SEQ ID NOs:1-59, or a fragment thereof.

B13. The method of embodiment B8, wherein at least one of the one or more loci in the first region comprises a nucleotide sequence selected from among SEQ ID NO:42, SEQ ID NO:52, SEQ ID NO:154, SEQ ID NO:158 and SEQ ID NO:163.

B14. The method of any one of embodiments 1 to B13, wherein the sequencing process is a sequencing by synthesis method.

B15. The method of any one of embodiments 1 to B13, wherein the sequencing process is a reversible terminator-based sequencing method.

B16. The method of any one of embodiments 1 to B15, wherein the amount of fetal nucleic acid determined is the fraction of fetal nucleic acid in the sample based on the amount of each of the fetal nucleic acid amplification products and total nucleic acid amplification products.

B17. The method of embodiment B16, wherein the fraction of fetal nucleic acid is a ratio of fetal nucleic acid amplification product amount to total nucleic acid amplification product amount.

B18. The method of any one of embodiments 1 to B17, further comprising contacting under amplification conditions the nucleic acid sample with a third set of amplification primers that amplify a third region in the sample nucleic acid allowing for a determination of the presence or absence of fetal specific nucleic acid.

B19. The method of embodiment B18, wherein the fetal specific nucleic acid is Y chromosome nucleic acid.

B20. The method of embodiment B19, wherein the third region comprises one or more loci within chromosome Y.

B21. The method of any one of embodiments 1 to B20, further comprising contacting under amplification conditions the nucleic acid sample with a fourth set of amplification primers that amplify a fourth region in the sample nucleic acid allowing for a determination of the amount of digested or undigested nucleic acid, as an indicator of digestion efficiency.

B22. The method of embodiment B21, wherein the fourth region comprises one or more loci present in both fetal nucleic acid and maternal nucleic acid and unmethylated in both fetal nucleic acid and maternal nucleic acid.

B23. The method of any one of embodiments 1 to B22, further comprising contacting under amplification conditions the nucleic acid sample with a predetermined copy number of one or more first competitor oligonucleotides that compete with the first region for hybridization of primers of the first amplification primer set.

B24. The method of any one of embodiments 1 to B23, further comprising contacting under amplification conditions the nucleic acid sample with a predetermined copy number of one or more second competitor oligonucleotides that compete with the second region for hybridization of primers of the second amplification primer set.

B25. The method of any one of embodiments 18 to B24, further comprising contacting under amplification conditions the nucleic acid sample with a predetermined copy number of one or more third competitor oligonucleotides that compete with the third region for hybridization of primers of the third amplification primer set.

B26. The method of any one of embodiments B21 to B25, further comprising contacting under amplification conditions the nucleic acid sample with a predetermined copy number of one or more fourth competitor oligonucleotides that compete with the fourth region for hybridization of primers of the fourth amplification primer set.

B27. The method of any one of embodiments B23 to B26, wherein the amount of fetal nucleic acid determined is the copy number of fetal nucleic acid based on the amount of competitor oligonucleotide used.

B28. The method of any one of embodiments 1 to B27, wherein the amount of fetal nucleic acid determined is the copy number of fetal nucleic acid based on a quantification of sequence reads.

B29. The method of any one of embodiments 1 to B28, wherein the sample nucleic acid is extracellular nucleic acid.

B30. The method of any one of embodiments 1 to B29, wherein the nucleic acid sample is obtained from a pregnant female subject.

B31. The method of embodiment B30, wherein the subject is human.

B32. The method of any one of embodiments 1 to B31, wherein the sample nucleic acid is from plasma or serum.

B33. The method of any one of embodiments 1 to B32, wherein two or more independent loci in the first region are assayed.

B34. The method of any one of embodiments 1 to B33, wherein the amount of fetal nucleic acid is substantially equal to the amount of fetal nucleic acid determined using a mass spectrometry method.

B35. The method of any one of embodiments 1 to B34, wherein the amount of fetal nucleic acid is determined with an R² value of 0.97 or greater when compared to an amount of fetal nucleic acid determined using a mass spectrometry method.

C1. A method for determining the copy number of fetal nucleic acid in a sample comprising:
(a) contacting a sample nucleic acid with one or more agents that differentially modify methylated nucleic acid and unmethylated nucleic acid, which sample nucleic acid comprises differentially methylated fetal nucleic acid and maternal nucleic acid, the combination of the fetal nucleic acid and the maternal nucleic acid comprising total nucleic acid in the sample, thereby generating differentially modified sample nucleic acid;
(b) contacting under amplification conditions the differentially modified sample nucleic acid with:
   (i) a first set of amplification primers that specifically amplify a first region in sample nucleic acid comprising one or more loci that are differentially methylated between the fetal nucleic acid and maternal nucleic acid, and
   (ii) a predetermined copy number of one or more first competitor oligonucleotides that compete with the first region for hybridization of primers of the first amplification primer set, thereby generating fetal nucleic acid amplification products and competitor amplification products;
(c) incorporating adaptor oligonucleotides into the amplification products in (b); thereby generating adaptor-modified amplification products;
(d) obtaining nucleotide sequences of the adaptor-modified amplification products in (c) by a sequencing process, thereby generating sequence reads;
(e) quantifying the sequence reads; and
(f) determining the copy number of fetal nucleic acid in the sample based on a quantification of the sequence reads in (e) and the amount of competitor oligonucleotide used.

C2. The method of embodiment C1, wherein the first region comprises one or more loci which each contain a restriction site for a methylation-sensitive restriction enzyme.

C3. The method of embodiment C2, wherein the one or more agents that differentially modify methylated nucleic acid and unmethylated nucleic acid comprise one or more methylation sensitive restriction enzymes.

C4. The method of embodiment C1, wherein the one or more agents that differentially modify methylated nucleic acid and unmethylated nucleic acid comprise bisulfite.

C5. The method of any one of embodiments C1 to C4, wherein the adaptor oligonucleotides are incorporated into the amplification products by ligation.

C6. The method of embodiment C5, wherein the ligation is unidirectional ligation.

C7. The method of any one of embodiments C1 to C4, wherein the adaptor oligonucleotides are incorporated into the amplification products using amplification primers comprising the adaptor oligonucleotide sequences.

C8. The method of any one of embodiments C1 to C7, wherein the adaptor oligonucleotides comprise one or more index sequences.

C9. The method of embodiment C8, wherein the one or more index sequences comprise a sample-specific index.

C10. The method of embodiment C8, wherein the one or more index sequences comprise an aliquot-specific index.

C11. The method of any one of embodiments C1 to C10, wherein at least one of the one or more loci in the first region comprises a nucleotide sequence selected from among SEQ ID NOs:1-261, or a fragment thereof.

C12. The method of embodiment C11, wherein at least one of the one or more loci in the first region comprises a nucleotide sequence selected from among SEQ ID NOs:1-89, or a fragment thereof.

C13. The method of embodiment C11, wherein at least one of the one or more loci in the first region comprises a nucleotide sequence selected from among SEQ ID NOs:90-261, or a fragment thereof.

C14. The method of embodiment C11, wherein at least one of the one or more loci in the first region comprises a nucleotide sequence selected from among SEQ ID NOs:1-59 and SEQ ID NOs:86-89, or a fragment thereof.

C15. The method of embodiment C11, wherein at least one of the one or more loci in the first region comprises a nucleotide sequence selected from among SEQ ID NOs:1-59, or a fragment thereof.

C16. The method of embodiment C11, wherein at least one of the one or more loci in the first region comprises a nucleotide sequence selected from among SEQ ID NO:42, SEQ ID NO:52, SEQ ID NO:154, SEQ ID NO:158 and SEQ ID NO:163.

C17. The method of any one of embodiments C1 to C16, wherein the sequencing process is a sequencing by synthesis method.

C18. The method of any one of embodiments C1 to C16, wherein the sequencing process is a reversible terminator-based sequencing method.

C19 The method of any one of embodiments C1 to C18, further comprising contacting under amplification conditions the nucleic acid sample with a second set of amplification primers that amplify a second region in the sample nucleic acid allowing for a determination of total nucleic acid in the sample, wherein the first region and the second region are different.

C20. The method of embodiment C19, wherein the second region comprises one or more loci which do not contain a restriction site for a methylation-sensitive restriction enzyme.

C21. The method of any one of embodiments C1 to C20, further comprising contacting under amplification conditions the nucleic acid sample with a third set of amplification primers that amplify a third region in the sample nucleic acid allowing for a determination of the presence or absence of fetal specific nucleic acid.

C22. The method of embodiment C21, wherein the fetal specific nucleic acid is Y chromosome nucleic acid.

C23. The method of embodiment C22, wherein the third region comprises one or more loci within chromosome Y.

C24. The method of any one of embodiments C3 to C23, further comprising contacting under amplification conditions the nucleic acid sample with a fourth set of amplification primers that amplify a fourth region in the sample nucleic acid allowing for a determination of the amount of digested or undigested nucleic acid, as an indicator of digestion efficiency.

C25. The method of embodiment C24, wherein the fourth region comprises one or more loci present in both fetal nucleic acid and maternal nucleic acid and unmethylated in both fetal nucleic acid and maternal nucleic acid.

C26. The method of any one of embodiments C19 to C25, further comprising contacting under amplification conditions the nucleic acid sample with a predetermined copy number of one or more second competitor oligonucleotides that compete with the second region for hybridization of primers of the second amplification primer set.

C27. The method of any one of embodiments C21 to C26, further comprising contacting under amplification conditions the nucleic acid sample with a predetermined copy number of one or more third competitor oligonucleotides that compete with the third region for hybridization of primers of the third amplification primer set.

C28. The method of any one of embodiments C24 to C27, further comprising contacting under amplification conditions the nucleic acid sample with a predetermined copy number of one or more fourth competitor oligonucleotides that compete with the fourth region for hybridization of primers of the fourth amplification primer set.

C29. The method of any one of embodiments C1 to C28, wherein the sample nucleic acid is extracellular nucleic acid.

C30. The method of any one of embodiments C1 to C29, wherein the nucleic acid sample is obtained from a pregnant female subject.

C31. The method of embodiment C30, wherein the subject is human.

C32. The method of any one of embodiments C1 to C31, wherein the sample nucleic acid is from plasma or serum.

C33. The method of any one of embodiments C1 to C32, wherein two or more independent loci in the first region are assayed.

C34. The method of any one of embodiments C1 to C33, wherein the copy number of fetal nucleic acid is substantially equal to the copy number of fetal nucleic acid determined using a mass spectrometry method.

C35. The method of any one of embodiments C1 to C34, wherein the copy number of fetal nucleic acid is determined with an R² value of 0.97 or greater when compared to a copy number of fetal nucleic acid determined using a mass spectrometry method.

D1. A method for detecting the presence or absence of a fetal aneuploidy in a sample comprising:
(a) contacting a sample nucleic acid with one or more agents that differentially modify methylated nucleic acid and unmethylated nucleic acid, which sample nucleic acid comprises differentially methylated fetal nucleic acid and maternal nucleic acid, the combination of the fetal nucleic acid and the maternal nucleic acid comprising total nucleic acid in the sample, thereby generating differentially modified sample nucleic acid;
(b) contacting under amplification conditions the differentially modified sample nucleic acid with:
   (i) a first set of amplification primers that specifically amplify one or more loci in a target chromosome that are differentially methylated between the fetal nucleic acid and maternal nucleic acid, and
   (ii) a second set of amplification primers that specifically amplify one or more loci in a reference chromosome that are differentially methylated between the fetal nucleic acid and maternal nucleic acid, thereby generating target chromosome amplification products and reference chromosome amplification products;
(c) incorporating adaptor oligonucleotides into the amplification products in (b); thereby generating adaptor-modified amplification products;
(d) obtaining nucleotide sequences of the adaptor-modified amplification products in (c) by a sequencing process, thereby generating sequence reads;
(e) quantifying the sequence reads; and
(f) detecting the presence or absence of a fetal aneuploidy in the sample based on a quantification of the sequence reads in (e).

D2. The method of embodiment D1, wherein the target chromosome comprises one or more loci which each contain a restriction site for a methylation-sensitive restriction enzyme.

D3. The method of embodiment D1 or D2, wherein the reference chromosome comprises one or more loci which each contain a restriction site for a methylation-sensitive restriction enzyme.

D4. The method of embodiment D2 or D3, wherein the one or more agents that differentially modify methylated nucleic acid and unmethylated nucleic acid comprise one or more methylation sensitive restriction enzymes.

D5. The method of embodiment D1, wherein the one or more agents that differentially modify methylated nucleic acid and unmethylated nucleic acid comprise bisulfite.

D6. The method of any one of embodiments D1 to D5, wherein the adaptor oligonucleotides are incorporated into the amplification products by ligation.

D7. The method of embodiment D6, wherein the ligation is unidirectional ligation.

D8. The method of any one of embodiments D1 to D5, wherein the adaptor oligonucleotides are incorporated into the amplification products using amplification primers comprising the adaptor oligonucleotide sequences.

D9. The method of any one of embodiments D1 to D8, wherein the adaptor oligonucleotides comprise one or more index sequences.

D10. The method of embodiment D9, wherein the one or more index sequences comprise a sample-specific index.

D11. The method of embodiment D9, wherein the one or more index sequences comprise an aliquot-specific index.

D12. The method of any one of embodiments D1 to D11, wherein at least one of the one or more loci in the target chromosome comprises a nucleotide sequence selected from among SEQ ID NOs:1-261, or a fragment thereof.

D13. The method of embodiment D12, wherein at least one of the one or more loci in the target chromosome comprises a nucleotide sequence selected from among SEQ ID NOs:1-89, or a fragment thereof.

D14. The method of embodiment D12, wherein at least one of the one or more loci in the target chromosome comprises a nucleotide sequence selected from among SEQ ID NOs:90-261, or a fragment thereof.

D15. The method of embodiment D12, wherein at least one of the one or more loci in target chromosome comprises a nucleotide sequence selected from among SEQ ID NOs:1-59 and SEQ ID NOs:86-89, or a fragment thereof.

D16. The method of embodiment D12, wherein at least one of the one or more loci in the target chromosome comprises a nucleotide sequence selected from among SEQ ID NOs:1-59, or a fragment thereof.

D17. The method of embodiment D12, wherein at least one of the one or more loci in the target chromosome comprises a nucleotide sequence selected from among SEQ ID NO:42, SEQ ID NO:52, SEQ ID NO:154, SEQ ID NO:158 and SEQ ID NO:163.

D18. The method of any one of embodiments D1 to D17, wherein at least one of the one or more loci in the reference chromosome comprises a nucleotide sequence selected from among SEQ ID NOs:1-261, or a fragment thereof.

D19. The method of embodiment D18, wherein at least one of the one or more loci in the reference chromosome comprises a nucleotide sequence selected from among SEQ ID NOs:1-89, or a fragment thereof.

D20. The method of embodiment D18, wherein at least one of the one or more loci in the reference chromosome comprises a nucleotide sequence selected from among SEQ ID NOs:90-261, or a fragment thereof.

D21. The method of embodiment D18, wherein at least one of the one or more loci in reference chromosome comprises a nucleotide sequence selected from among SEQ ID NOs:1-59 and SEQ ID NOs:86-89, or a fragment thereof.

D22. The method of embodiment D18, wherein at least one of the one or more loci in the reference chromosome comprises a nucleotide sequence selected from among SEQ ID NOs:1-59, or a fragment thereof.

D23. The method of embodiment D18, wherein at least one of the one or more loci in the reference chromosome comprises a nucleotide sequence selected from among SEQ ID NO:42, SEQ ID NO:52, SEQ ID NO:154, SEQ ID NO:158 and SEQ ID NO:163.

D24. The method of any one of embodiments D1 to D23, wherein the sequencing process is a sequencing by synthesis method.

D25. The method of any one of embodiments D1 to D23, wherein the sequencing process is a reversible terminator-based sequencing method.

D26. The method of any one of embodiments D1 to D25, further comprising contacting under amplification conditions the nucleic acid sample with a predetermined copy number of one or more first competitor oligonucleotides that compete with the target chromosome for hybridization of primers of the first amplification primer set.

D27. The method of any one of embodiments D1 to D26, further comprising contacting under amplification conditions the nucleic acid sample with a predetermined copy number of one or more second competitor oligonucleotides that compete with the reference chromosome for hybridization of primers of the second amplification primer set.

D28. The method of any one of embodiments D1 to D27, wherein the sample nucleic acid is extracellular nucleic acid.

D29. The method of any one of embodiments D1 to D28, wherein the nucleic acid sample is obtained from a pregnant female subject.

D30. The method of embodiment D29, wherein the subject is human.

D31. The method of any one of embodiments D1 to D30, wherein the sample nucleic acid is from plasma or serum.

D32. The method of any one of embodiments D1 to D31, wherein two or more independent loci in the target chromosome are assayed.

D33. The method of any one of embodiments D1 to D32, wherein two or more independent loci in the reference chromosome are assayed.

D34. The method of any one of embodiments D1 to D33, wherein the target chromosome is chromosome 13.

D35. The method of any one of embodiments D1 to D33, wherein the target chromosome is chromosome 18.

D36. The method of any one of embodiments D1 to D33, wherein the target chromosome is chromosome 21.

E1. A method for determining fetal fraction in a sample comprising:
(a) enriching a sample nucleic acid for a plurality of polymorphic nucleic acid targets, which sample nucleic acid comprises fetal nucleic acid and maternal nucleic acid;
(b) obtaining nucleotide sequences for some or all of the nucleic acid targets by a sequencing process;
(c) analyzing the nucleotide sequences of (b); and
(d) determining fetal fraction based on the analysis of (c), wherein the polymorphic nucleic acid targets and number thereof result in at least five polymorphic nucleic acid targets being informative for determining the fetal fraction for at least 90% of samples.

E2. The method of embodiment E1, wherein the enriching comprises amplifying the plurality of polymorphic nucleic acid targets.

E3. The method of embodiment E1 or E2, wherein the enriching comprises generating amplification products in an amplification reaction.

E4. The method of embodiment E3, wherein the amplification reaction is performed in a single vessel.

E5. The method of any one of embodiments E1 to E4, wherein the maternal genotype and the paternal genotype at each of the polymorphic nucleic acid targets are not known prior to (a).

E5.1 The method of any one of embodiments E1 to E5, wherein polymorphic nucleic acid targets having a minor allele population frequency of about 40% or more are selected.

E6. The method of any one of embodiments E1 to E5.1, comprising determining an allele frequency in the sample for each of the polymorphic nucleic acid targets.

E7. The method of embodiment E6, wherein determining which polymorphic nucleic acid targets are informative comprises identifying informative genotypes by comparing each allele frequency to one or more fixed cutoff frequencies.

E7.1 The method of embodiment E7, wherein the fixed cutoff for identifying informative genotypes from non-informative homozygotes is about a 1% or greater shift in allele frequency.

E7.2 The method of embodiment E7, wherein the fixed cutoff for identifying informative genotypes from non-informative homozygotes is about a 2% or greater shift in allele frequency.

E7.3 The method of embodiment E7, wherein the fixed cutoff for identifying informative genotypes from non-informative heterozygotes is about a 25% or greater shift in allele frequency.

E7.4 The method of embodiment E7, wherein the fixed cutoff for identifying informative genotypes from non-informative heterozygotes is about a 50% or greater shift in allele frequency.

E8. The method of embodiment E6, wherein determining which polymorphic nucleic acid targets are informative comprises identifying informative genotypes by comparing each allele frequency to one or more target-specific cutoff frequencies.

E9. The method of embodiment E8, wherein the one or more target-specific cutoff frequencies are determined for each polymorphic nucleic acid target.

E10. The method of embodiment E8 or E9, wherein each target-specific cutoff frequency is determined based on the allele frequency variance for the corresponding polymorphic nucleic acid target.

E11. The method of any one of embodiments E6 to E10, further comprising determining an allele frequency mean.

E12. The method of embodiment E11, wherein fetal fraction is determined based, in part, on the allele frequency mean.

E13. The method of any one of embodiments E1 to E12, wherein the fetal genotype at one or more informative polymorphic nucleic acid targets is heterozygous.

E14. The method of any one of embodiments E1 to E13, wherein the fetal genotype at one or more informative polymorphic nucleic acid targets is homozygous.

E15. The method of any one of embodiments E1 to E14, wherein fetal fraction is determined with a coefficient of variance (CV) of 0.20 or less.

E16. The method of embodiment E15, wherein fetal fraction is determined with a coefficient of variance (CV) of 0.10 or less.

E17. The method of embodiment E16, wherein fetal fraction is determined with a coefficient of variance (CV) of 0.05 or less.

E18. The method of any one of embodiments E1 to E17, wherein the polymorphic nucleic acid targets each comprise at least one single nucleotide polymorphism (SNP).

E19. The method of embodiment E18, wherein the SNPs are selected from: rs10413687, rs10949838, rs1115649, rs11207002, rs11632601, rs11971741, rs12660563, rs13155942, rs1444647, rs1572801, rs17773922, rs1797700, rs1921681, rs1958312, rs196008, rs2001778, rs2323659, rs2427099, rs243992, rs251344, rs254264, rs2827530, rs290387, rs321949, rs348971, rs390316, rs3944117, rs425002, rs432586, rs444016, rs4453265, rs447247, rs4745577, rs484312, rs499946, rs500090, rs500399, rs505349, rs505662, rs516084, rs517316, rs517914, rs522810, rs531423, rs537330, rs539344, rs551372, rs567681, rs585487, rs600933, rs619208, rs622994, rs639298, rs642449, rs6700732, rs677866, rs683922, rs686851, rs6941942, rs7045684, rs7176924, rs7525374, rs870429, rs949312, rs9563831, rs970022, rs985462, rs1005241, rs1006101, rs10745725, rs10776856, rs10790342, rs11076499, rs11103233, rs11133637, rs11974817, rs12102203, rs12261, rs12460763, rs12543040, rs12695642, rs13137088, rs13139573, rs1327501, rs13438255, rs1360258, rs1421062, rs1432515, rs1452396, rs1518040, rs16853186, rs1712497, rs1792205, rs1863452, rs1991899, rs2022958, rs2099875, rs2108825, rs2132237, rs2195979, rs2248173, rs2250246, rs2268697, rs2270893, rs244887, rs2736966, rs2851428, rs2906237, rs2929724, rs3742257, rs3764584, rs3814332, rs4131376, rs4363444, rs4461567, rs4467511, rs4559013, rs4714802, rs4775899, rs4817609, rs488446, rs4950877, rs530913, rs6020434, rs6442703, rs6487229, rs6537064, rs654065, rs6576533, rs6661105, rs669161, rs6703320, rs675828, rs6814242, rs6989344, rs7120590, rs7131676, rs7214164, rs747583, rs768255, rs768708, rs7828904, rs7899772, rs7900911, rs7925270, rs7975781, rs8111589, rs849084, rs873870, rs9386151, rs9504197, rs9690525, and rs9909561.

E20. The method of embodiment E19, wherein the SNPs are selected from: rs10413687, rs10949838, rs1115649, rs11207002, rs11632601, rs11971741, rs12660563, rs13155942, rs1444647, rs1572801, rs17773922, rs1797700, rs1921681, rs1958312, rs196008, rs2001778, rs2323659, rs2427099, rs243992, rs251344, rs254264, rs2827530, rs290387, rs321949, rs348971, rs390316, rs3944117, rs425002, rs432586, rs444016, rs4453265, rs447247, rs4745577, rs484312, rs499946, rs500090, rs500399, rs505349, rs505662, rs516084, rs517316, rs517914, rs522810, rs531423, rs537330, rs539344, rs551372, rs567681, rs585487, rs600933, rs619208, rs622994, rs639298, rs642449, rs6700732, rs677866, rs683922, rs686851, rs6941942, rs7045684, rs7176924, rs7525374, rs870429, rs949312, rs9563831, rs970022, and rs985462.

E21. The method of embodiment E19, wherein the SNPs are selected from: rs1005241, rs1006101, rs10745725, rs10776856, rs10790342, rs11076499, rs11103233, rs11133637, rs11974817, rs12102203, rs12261, rs12460763, rs12543040, rs12695642, rs13137088, rs13139573, rs1327501, rs13438255, rs1360258, rs1421062, rs1432515, rs1452396, rs1518040, rs16853186, rs1712497, rs1792205, rs1863452, rs1991899, rs2022958, rs2099875, rs2108825, rs2132237, rs2195979, rs2248173, rs2250246, rs2268697, rs2270893, rs244887, rs2736966, rs2851428, rs2906237, rs2929724, rs3742257, rs3764584, rs3814332, rs4131376, rs4363444, rs4461567, rs4467511, rs4559013, rs4714802, rs4775899, rs4817609, rs488446, rs4950877, rs530913, rs6020434, rs6442703, rs6487229, rs6537064, rs654065, rs6576533, rs6661105, rs669161, rs6703320, rs675828, rs6814242, rs6989344, rs7120590, rs7131676, rs7214164, rs747583, rs768255, rs768708, rs7828904, rs7899772, rs7900911, rs7925270, rs7975781, rs8111589, rs849084, rs873870, rs9386151, rs9504197, rs9690525, and rs9909561.

E22. The method of any one of embodiments E1 to E21, wherein the polymorphic nucleic acid targets and number thereof result in at least five polymorphic nucleic acid targets being informative for determining the fetal fraction for at least 95% of samples.

E23. The method of embodiment E22, wherein the polymorphic nucleic acid targets and number thereof result in at least five polymorphic nucleic acid targets being informative for determining the fetal fraction for at least 99% of samples.

E24. The method of any one of embodiments E1 to E21, wherein the polymorphic nucleic acid targets and number thereof result in at least ten polymorphic nucleic acid targets being informative for determining the fetal fraction for at least 90% of samples.

E25. The method of embodiment E24, wherein the polymorphic nucleic acid targets and number thereof result in at least ten polymorphic nucleic acid targets being informative for determining the fetal fraction for at least 95% of samples.

E26. The method of embodiment E25, wherein the polymorphic nucleic acid targets and number thereof result in at least ten polymorphic nucleic acid targets being informative for determining the fetal fraction for at least 99% of samples.

E27. The method of any one of embodiments E1 to E26, wherein 10 or more polymorphic nucleic acid targets are enriched.

E27.1 The method of embodiment E27, wherein about 40 to about 100 polymorphic nucleic acid targets are enriched.

E28. The method of embodiment E27, wherein 50 or more polymorphic nucleic acid targets are enriched.

E29. The method of embodiment E28, wherein 100 or more polymorphic nucleic acid targets are enriched.

E30. The method of embodiment E29, wherein 500 or more polymorphic nucleic acid targets are enriched.

E31. The method of any one of embodiments E1 to E30, wherein the sequencing process comprises a sequencing by synthesis method.

E31.1 The method of embodiment E31, wherein the sequencing by synthesis method comprises a plurality of synthesis cycles.

E31.2 The method of embodiment E31.1, wherein the sequencing by synthesis method comprises about 36 cycles.

E31.3 The method of embodiment E31.1, wherein the sequencing by synthesis method comprises about 27 cycles.

E32. The method of any one of embodiments E1 to E30, wherein the sequencing process comprises a sequencing by ligation method.

E33. The method of any one of embodiments E1 to E30, wherein the sequencing process comprises a single molecule sequencing method.

E34. The method of any one of embodiments E1 to E33, wherein the sequencing process comprises sequencing a plurality of samples in a single compartment.

E35. The method of embodiment E34, wherein the fetal fraction is determined for 10 or more samples.

E36. The method of embodiment E35, wherein the fetal fraction is determined for 100 or more samples.

E37. The method of embodiment E36, wherein the fetal fraction is determined for 1000 or more samples.

E38. The method of any one of embodiments E1 to E37, wherein the sample nucleic acid is cell-free DNA.

E39. The method of any one of embodiments E1 to E38, wherein the sample nucleic acid is obtained from a pregnant female subject.

E40. The method of embodiment E39, wherein the subject is human.

E41. The method of any one of embodiments E1 to E40, wherein the sample nucleic acid is from plasma or serum.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

1. (canceled)

2. A method for detecting the presence or absence of a chromosomal abnormality by analyzing the amount or copy number of a target nucleic acid and a control nucleic acid from a sample of differentially methylated nucleic acids comprising the steps of:
(a) enriching both a target nucleic acid and a control nucleic acid from the sample based on a methylation state for both the target and control nucleic acid;
(b) performing a copy number analysis of the enriched target nucleic acid in at least one of the fractions;
(c) performing a copy number analysis of the enriched control nucleic acid in at least one of the fractions;
(d) comparing the copy number from step (b) with the copy number from step (c); and
(e) determining if a chromosomal abnormality exists based on the comparison in step (d), wherein the target nucleic acid and control nucleic acid have the same or substantially the same methylation status.

3. The method of claim 2, wherein step (a) comprises contacting the target nucleic acid and the control nucleic acid with one or more agents that differentially modify methylated nucleic acid and unmethylated nucleic acid.

4. The method of claim 3, wherein the one or more agents that differentially modify methylated nucleic acid and unmethylated nucleic acid comprises a methylation sensitive restriction enzyme.

5. The method of claim 3, wherein the one or more agents that differentially modify methylated nucleic acid and unmethylated nucleic acid comprises bisulfite.

6. The method of claim 2, further comprising:
incorporating adaptor oligonucleotides into the amplification products, thereby generating adaptor-modified amplification products;
obtaining nucleotide sequences of the adaptor-modified amplification products by a sequencing process, thereby generating sequence reads; and
quantifying the sequence reads.

7. The method of claim 6, wherein the adaptor oligonucleotides comprise one or more sample-specific index sequences.

8. The method of claim 2, wherein the sample nucleic acid is extracellular nucleic acid.

9. The method of claim 2, wherein the sample nucleic acid comprises cancer nucleic acid.

10. The method of claim 2, wherein the enriched target nucleic acid nucleic acid of step (a) comprises at least one locus set forth in any one of SEQ ID NOs: 1-261.

11. The method of claim 10, wherein the enriched target nucleic acid of step (a) comprises SEQ ID No. 13, SEQ ID No. 33, SEQ ID No. 52, SEQ ID No. 73, SEQ ID No. 142, SEQ ID No. 195, or SEQ ID No. 196.

12. The method of claim 2, wherein the chromosomal abnormality is associated with cancer.
